(19) United States
(12) Patent Application Publication
(10) Pub. No.: US 2024/0188424 A1
(43) Pub. Date: Jun. 6, 2024

(54) ORGANIC ELECTROLUMINESCENT COMPOUND, A PLURALITY OF HOST MATERIALS AND ORGANIC ELECTROLUMINESCENT DEVICE COMPRISING THE SAME

(71) Applicant: ROHM AND HAAS ELECTRONIC MATERIALS KOREA LTD., Chungchengnam-do (KR)

(72) Inventors: Su-Hyun LEE, Gyeonggi-do (KR); Kyung-Hoon CHOI, Gyeonggi-do (KR); So-Young JUNG, Gyeonggi-do (KR); Chi-Sik KIM, Gyeonggi-do (KR); Soo-Yong LEE, Gyeonggi-do (KR); Sang-Hee CHO, Gyeonggi-do (KR); Young-Jun CHO, Gyeonggi-do (KR); Young-Gil KIM, Gyeonggi-do (KR); Bitnari KIM, Gyeonggi-do (KR); Young-Jae KIM, Gyeonggi-do (KR); Hyo-Nim SHIN, Gyeonggi-do (KR)

(21) Appl. No.: 18/379,813

(22) Filed: Oct. 13, 2023

(30) Foreign Application Priority Data
Oct. 14, 2022 (KR) 10-2022-0132343
Jun. 26, 2023 (KR) 10-2023-0081999
Oct. 6, 2023 (KR) 10-2023-0133347

(51) Int. Cl. H10K 85/60; C07D 307/77; C07D 307/91; C07D 405/04; C07D 405/12; C07D 413/12; C09K 11/06

(52) U.S. Cl. CPC H10K 85/636; C07D 307/77; C07D 307/91; C07D 405/04; C07D 405/12; C07D 413/12; C09K 11/06; H10K 85/622; H10K 85/626; H10K 85/633; H10K 85/654; H10K 85/6574; H10K 50/11

(57) ABSTRACT

The present disclosure relates to an organic electroluminescent compound, a plurality of host materials, and an organic electroluminescent device comprising the same. By comprising the organic electroluminescent compound according to the present disclosure or by comprising a specific combination of compounds according to the present disclosure as a plurality of host materials, it is possible to produce an organic electroluminescent device having improved luminous efficiency, and/or lifetime properties compared to the conventional organic electroluminescent devices.

### TECHNICAL FIELD

The present disclosure relates to an organic electroluminescent compound, a plurality of host materials, and an organic electroluminescent device comprising the same.

### BACKGROUND ART

A small molecular green organic electroluminescent device (OLED) was first developed by Tang, et al., of Eastman Kodak in 1987 by using TPD/ALq3 bi-layer consisting of a light-emitting layer and a charge transport layer. Thereafter, the development of OLEDs was rapidly effected and OLEDs have been commercialized. At present, OLEDs primarily use phosphorescent materials having excellent luminous efficiency in panel implementation. However, in many applications such as TVs and lightings, the lifetime of OLEDs is insufficient and higher efficiency of OLEDs is still required. Typically, the higher the luminance of an OLED, the shorter the lifetime that the OLED has. Therefore, an OLED having high luminous efficiency and/or long lifetime properties is required for long time use and high resolution of a display.

In order to enhance luminous efficiency, driving voltage and/or lifetime, various materials or concepts for an organic layer of an OLED have been proposed. However, they were not satisfied in practical use. In addition, there has been a need to develop an organic electroluminescent material having more improved performances, for example, improved driving voltage, luminous efficiency, power efficiency, and/or lifetime properties compared to a combination of specific compounds previously disclosed.

Meanwhile, Korean Patent Application Laying-Open No. 2017-0022865 discloses an organic electroluminescent device using phenanthrooxazole and phenanthrothiazole compounds as hosts. However, the aforementioned reference does not specifically disclose an organic electroluminescent device using a specific compound or a specific combination of a plurality of host materials claimed herein, and the development of host materials to improve OLED performance is still required.

### DISCLOSURE OF INVENTION

### Technical Problem

The objective of the present disclosure is to provide an organic electroluminescent compound having a new structure suitable for applying to an organic electroluminescent device. Another objective of the present disclosure is to provide an organic electroluminescent device having higher luminous efficiency and/or improved lifetime properties by comprising a plurality of host materials comprising a specific combination of compounds.

### Solution to Problem

The present inventors noted that compounds having a core such as phenanthrooxazole, phenanthrothiazole, etc. uniquely have a lower LUMO (lowest unoccupied molecular orbital) energy level in compared to typical hole-type hosts, and studied a hole-type host capable of forming an appropriate energy gap with the compound. As a result, when a combination of the compound represented by the following formula 1 and the compound represented by the following formula 2 is used in a light-emitting layer, hole and electron properties are balanced by appropriate HOMO and LUMO energy levels, and it is possible to provide an organic electroluminescent device having higher luminous efficiency and/or longer lifetime compared to the conventional organic electroluminescent device.

Specifically, the present inventors found that the above objective can be achieved by the compound represented by the following formula 1′ and comprising at least one deuterium. In addition, a result of intensive studies to solve the technical problems, the present inventors found that the above objective can be achieved by a plurality of host materials comprising a first host material comprising at least one compound represented by the following formula 1 and a second host material comprising at least one compound represented by the following formula 2, wherein at least one of the first host material and the second host material comprises deuterium.

Furthermore, in the plurality of host materials, the present inventors found that the above objective can be achieved by a plurality of host materials further comprising a third host material.

For example, in the plurality of host materials further comprising the third host material, the present inventors found that the above objective can be achieved by the plurality of host materials, wherein the third host material comprises the compound represented by at least one of the following formula 3.

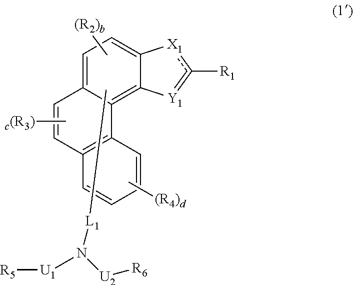

In formula 1′,
X₁ and Y₁ each independently represent —N═, —NR₇—, —O— or —S—, with a proviso that one of X₁ and Y₁ represents —N═, and the other of X₁ and Y₁ represents —NR₇—, —O— or —S—;
R₁ represents a substituted or unsubstituted (C6-C30)aryl, or a substituted or unsubstituted (3- to 30-membered)heteroaryl;
R₂ to R₇ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino, or may be linked to an adjacent substituent(s) to form a ring(s);
L₁, U₁, and U₂ each independently represent a single bond, a substituted or unsubstituted (C6-C30)arylene, or a substituted or unsubstituted (3- to 30-membered)heteroarylene;
b and c each independently represent an integer of 1 or 2, d represents an integer of 1 to 4, where if b to d represent an integer of 2 or more, each of R₂ to each of R₄ may be the same as or different from each other; and
in formula 1′, at least one of deuterium is included.

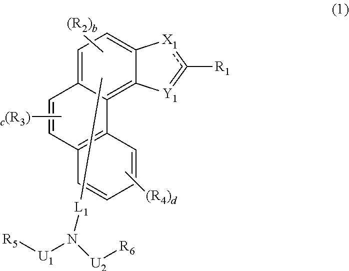

In formula 1,
X₁ and Y₁ each independently represent —N═, —NR₇—, —O— or —S—, with a proviso that one of X₁ and Y₁ represents —N═, the other of X₁ and Y₁ represents —NR₇—, —O— or —S—;
R₁ represents a substituted or unsubstituted (C6-C30)aryl, or a substituted or unsubstituted (3- to 30-membered)heteroaryl;
R₂ to R₇ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino, or may be linked to an adjacent substituent(s) to form a ring(s);
L₁, U₁, and U₂ each independently represent a single bond, a substituted or unsubstituted (C6-C30)arylene, or a substituted or unsubstituted (3- to 30-membered)heteroarylene; and
b and c each independently represent an integer of 1 or 2, d represents an integer of 1 to 4, where if b to d represent an integer of 2 or more, each of R₂ to each of R₄ may be the same as or different from each other.

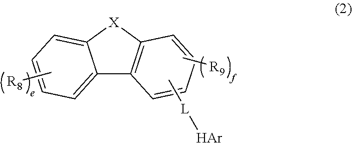

In formula 2,
X represents O or S;
HAr represents a substituted or unsubstituted (3- to 30-membered)heteroaryl, including at least one nitrogen atom;
L represents a single bond, a substituted or unsubstituted (C6-C30)arylene, or a substituted or unsubstituted (3- to 30-membered)heteroarylene;
R₃ and R₉ each independently represent hydrogen, deuterium, a substituted or unsubstituted (C6-C30)aryl, a substituted or unsubstituted (3- to 30-membered)heteroaryl, or —SiR₂₁R₂₂R₂₃;
R₂₁ to R₂₃ each independently represent a substituted or unsubstituted (C1-C30)alkyl, a substituted or unsubstituted (C6-C30)aryl, or a substituted or unsubstituted (3- to 30-membered)heteroaryl; and
e represents an integer of 1 to 4, and f represents an integer of 1 to 3, where if e and f represent an integer of 2 or more, each of R₃ and each of R₉ may be the same as or different from each other.

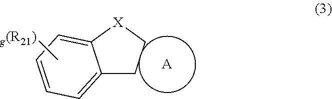

In formula 3,
X represents O, S, CR₃₁R₃₂, NR₃₃, or Se;
R₃₁ to R₃₃ each independently represent hydrogen, deuterium, halogen, a substituted or unsubstituted (C1-C30)alkyl, a substituted or unsubstituted (C6-C30)aryl, or a substituted or unsubstituted (3- to 30-membered)heteroaryl; or may be linked to an adjacent substituent(s) to form a ring(s); A represents a substituted or unsubstituted phenanthrene ring represented by the following formula 3-1;

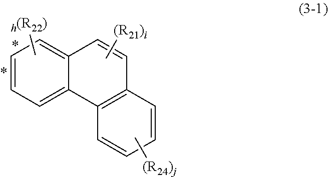

in formulas 3 and 3-1,
R₂₁ to R₂₄ each independently represent hydrogen, deuterium, a halogen, a substituted or unsubstituted (C1-C30)alkyl, a substituted or unsubstituted (C6-C30)aryl,

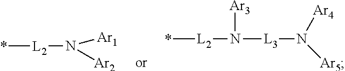

with a proviso that at least one of R₂₁ to R₂₄ represents

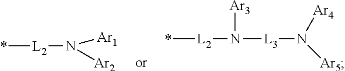

L₂ and L₃ each independently represent a single bond, a substituted or unsubstituted (C6-C30)arylene, a substituted or unsubstituted (C3-C30)cycloalkylene, or a substituted or unsubstituted (3- to 30-membered)heteroarylene;
Ar₁ to Ar₅ each independently represent a substituted or unsubstituted (C6-C30)aryl or a substituted or unsubstituted (3- to 30-membered)heteroaryl;
g and j represent an integer of 1 to 4, h and i represent an integer of 1 or 2, where if g to h represent an integer of 2 or more, each of R₂₁ to each of R₂₄ may be the same as or different from each other;
   in formula 3-1 represents a site linked to formula 3; and
   in R₂₁ to R₂₄ represents a site linked to formula 3 or 3-1.

### Advantageous Effects of Invention

An organic electroluminescent compound according to the present disclosure exhibits performance suitable for use in an organic electroluminescent device. In addition, an organic electroluminescent device having higher luminous efficiency and/or improved lifetime properties compared to conventional organic electroluminescent devices is provided by comprising the compound according to the present disclosure as a single host material, or by comprising the plurality of host materials according to the present disclosure, and it is possible to produce a display system or a lighting system using the same.

### MODE FOR THE INVENTION

Hereinafter, the present disclosure will be described in detail. However, the following description is intended to explain the present disclosure and is not meant in any way to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “an organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of host materials” in the present disclosure means a host material comprising a combination of at least two compounds, which may be comprised in any light-emitting layer constituting an organic electroluminescent device. It may mean both a material before being comprised in an organic electroluminescent device (for example, before vapor deposition) and a material after being comprised in an organic electroluminescent device (for example, after vapor deposition). For example, the plurality of host materials of the present disclosure is a combination of at least two host materials, and may selectively further comprise conventional materials comprised in an organic electroluminescent material. At least two compounds comprised in the plurality of host materials of the present disclosure may be comprised together in one light-emitting layer or may respectively be comprised in different light-emitting layers. For example, the at least two host materials may be mixture-evaporated or co-evaporated, or may be separately evaporated.

Herein, the term “(C1-C30)alkyl” is meant to be a linear or branched alkyl having 1 to 30 carbon atoms constituting the chain, in which the number of carbon atoms is preferably 1 to 10, and more preferably 1 to 6. The above alkyl may include methyl, ethyl, n-propyl, isopropyl, n-butyl, isobutyl, tert-butyl, sec-butyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3- to 7-membered)heterocycloalkyl” is meant to be a cycloalkyl having 3 to 7 ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl” or “(C6-C30)arylene” is meant to be a monocyclic or fused ring radical derived from an aromatic hydrocarbon having 6 to 30 ring backbone carbon atoms. The above aryl may be partially saturated, and may comprise a spiro structure. The above aryl may include phenyl, biphenyl, terphenyl, quinquephenyl, naphthyl, binaphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, benzophenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluoren]yl, spiro[cyclopentene-fluoren]yl, spiro[dihydroindene-fluoren]yl, azulenyl, tetramethyldihydrophenanthrenyl, etc. Specifically, the above aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenyl, 3-biphenyl, 4-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl” or “(3- to 30-membered)heteroarylene” is meant to be an aryl group having 3 to 30 ring backbone atoms, and including at least one, preferably 1 to 4 heteroatoms selected from the group consisting of B, N, O, S, Si, and P. The above heteroaryl may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, isobenzofuranyl, dibenzofuranyl, dibenzothiophenyl, dibenzoselenophenyl, naphthobenzofuranyl, naphthobenzothiophenyl, naphthooxazolyl, benzofuroquinolinyl, benzofuroquinazolinyl, benzofuronaphthyridinyl, benzofuropyrimidinyl, naphthofuropyrimidinyl, benzothienoquinolyl, benzothienoquinazolinyl, naphthyridinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, benzoquinazolinyl, quinoxalinyl, benzoquinoxalinyl, carbazolyl, benzocarbazolyl, dibenzocarbazolyl, phenoxazinyl, phenanthridinyl, benzodioxolyl, dihydroacridinyl, benzotriazolyl, phenazinyl, imidazopyridyl, chromenoquinazolinyl, thiochromenoquinazolinyl, dimethylbenzoperimidinyl, indolocarbazolyl, indenocarbazolyl, etc. More specifically, the above heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. “Heteroaryl(ene)” can be classified into heteroaryl(ene) with electronic properties and heteroaryl(ene) with hole properties. Heteroaryl(ene) with electronic properties is a substituent which is relatively rich in electrons in the parent nucleus, for example, a substituted or unsubstituted pyridinyl, a substituted or unsubstituted pyrimidinyl, a substituted or unsubstituted triazinyl, a substituted or unsubstituted quinazolinyl, a substituted or unsubstituted quinoxalinyl, or a substituted or unsubstituted quinolyl, etc. Heteroaryl(ene) with hole properties is a substituent which is relatively poor in electrons in the parent nucleus, for example, a substituted or unsubstituted carbazolyl, a substituted or unsubstituted dibenzofuranyl, a substituted or unsubstituted dibenzothiophenyl, etc. Furthermore, “halogen” includes F, Cl, Br, and I.

In addition, “ortho (o-),” “meta (m-),” and “para (p-)” are prefixes, which represent the relative positions of substituents respectively. Ortho indicates that two substituents are adjacent to each other, and for example, when two substituents in a benzene derivative occupy positions 1 and 2 or positions 2 and 3, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

Unless otherwise specified, the substituent may replace hydrogen at a position where the substituent can be substituted without limitation, and when two or more hydrogen atoms in a certain functional group are each replaced with a substituent, each substituent may be the same or different from each other. The maximum number of substituents that can be substituted for a certain functional group may be the total number of valences that can be substituted for each atom forming the functional group. Herein, the substituted aryl, the substituted heteroaryl, the substituted arylene, the substituted heteroarylene, the substituted alkyl, the substituted cycloalkyl, the substituted alkoxy, the substituted trialkylsilyl, the substituted dialkylarylsilyl, the substituted alkyldiarylsilyl, the substituted triarylsilyl, the substituted fused ring group of an aliphatic ring(s) and an aromatic ring(s), the substituted mono- or di-alkylamino, the substituted mono- or di-alkenylamino, the substituted alkylalkenylamino, the substituted mono- or di-arylamino, the substituted alkylarylamino, the substituted mono- or di-heteroarylamino, the substituted alkylheteroarylamino, the substituted alkenylarylamino, the substituted alkenylheteroarylamino, and the substituted arylheteroarylamino, each independently, are substituted by at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of deuterium and a (C6-C30)aryl(s); a (C6-C30)aryl unsubstituted or substituted with at least one of deuterium and a (C6-C30)aryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a (C1-C30)alkyl(C2-C30)alkenylamino; a mono- or di-(C6-C30)arylamino; a (C1-C30)alkyl(C6-C30)arylamino; a mono- or di-(3- to 30-membered)heteroarylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituted alkyl, etc., each independently, are substituted by at least one selected from the group consisting of deuterium; a (C1-C10)alkyl unsubstituted or substituted with deuterium; a (C6-C22)aryl unsubstituted or substituted with at least one of deuterium and a (C6-C18)aryl(s); a (6- to 20-membered)heteroaryl unsubstituted or substituted with deuterium; and a tri(C6-C15)arylsilyl unsubstituted or substituted with deuterium. According to another embodiment of the present disclosure, the substituted alkyl, etc., each independently, are substituted by at least one selected from the group consisting of deuterium; a (C1-C6)alkyl unsubstituted or substituted with deuterium; a (C6-C18)aryl unsubstituted or substituted with at least one of deuterium and a (C6-C18)aryl(s); a (6- to 15-membered)heteroaryl unsubstituted or substituted with deuterium; and a tri(C6-C10)arylsilyl unsubstituted or substituted with deuterium. For example, the substituted alkyl, etc., may be substituted by deuterium or at least one selected from the group consisting of a methyl, a tetramethyl, a phenyl, a biphenyl, a naphthyl, a phenylnaphthyl, a naphthylphenyl, a terphenyl, a naphthyl substituted with a biphenyl(s), a phenanthrenyl, a benzo[c]phenanthrenyl, a chrysenyl, a pyridinyl, a dibenzofuranyl, a triphenylsilyl, a carbazolyl, a phenylcarbazolyl, a nitrilphenyl, a nitrile, a fluorenyl, an adamantyl, and a fluorenyl substituted with a methyl(s), in which the substituents may be further substituted with deuterium.

If a substituent is not indicated in the chemical formula or compound structure herein, it may mean that all positions that can be substituted for the substituent are hydrogen or deuterium. That is, in the case of deuterium, it is an isotope of hydrogen, and some hydrogen atoms may be the isotope deuterium, and in this case, the content of deuterium may be 0% to 100%. In cases where substituents are not indicated in the chemical formula or compound structure herein, if deuterium is not explicitly excluded, for example the content of deuterium is 0%, the content of hydrogen is 100%, all substituents are hydrogen, hydrogen and deuterium can be used together in the compound. The deuterium is one of the isotopes of hydrogen and is an element that has a deuteron, consisting of one proton and one neutron, as its nucleus. It can be expressed as hydrogen-2, and its element symbol can be written as D or 2H. The isotopes refer to atoms with the same atomic number (Z) but different mass numbers (A), and can also be interpreted as elements with the same number of protons but different numbers of neutrons.

The term “a combination thereof” in the present disclosure refers to a combination of one or more elements from the corresponding list to form a known or chemically stable arrangement that can be envisioned by one skilled in the art from the corresponding list. For example, alkyl and deuterium can be combined to form a partially or fully deuterated alkyl group; halogen and alkyl can be combined to form a halogenated alkyl substituent; halogen, alkyl, and aryl can be combined to form a halogenated arylalkyl. For example, preferred combinations of substituents include up to 50 atoms that are not hydrogen or deuterium, or up to 40 atoms that are not hydrogen or deuterium, or up to 30 atoms that are not hydrogen or deuterium. Alternatively, in many cases, preferred combinations of substituents may include up to 20 atoms that are not hydrogen or deuterium.

In the formulas of the present disclosure, when a ring is formed by a linkage of adjacent substituents, the ring may be a substituted or unsubstituted, mono- or polycyclic, (3- to 30-membered) alicyclic or aromatic ring, or the combination thereof, which is formed by linkage of at least two adjacent substituents. In addition, the formed ring may contain at least one heteroatom selected from B, N, O, S, Si, and P, preferably at least one heteroatom selected from N, O, and S. According to one embodiment of the present disclosure, the number of the ring backbone atoms is 5 to 20, and according to another embodiment of the present disclosure, the number of the ring backbone atoms is 5 to 15.

In the formulas of the present disclosure, heteroaryl or heteroarylene may, each independently, contain at least one heteroatom selected from B, N, O, S, Si, and P. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (5- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

Hereinafter, an organic electroluminescent compound according to one embodiment of the present disclosure will be described.

The organic electroluminescent compound according to one embodiment of the present disclosure is represented by the following formula 1′ and contains one or more deuterium.

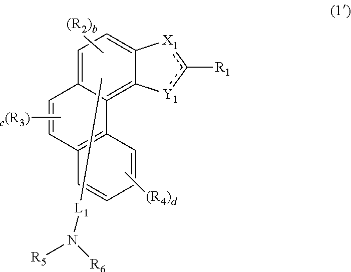

In formula 1′,
X₁ and Y₁ each independently represent —N═, —NR₇—, —O— or —S—, with a proviso that one of X₁ and Y₁ represents —N═, and the other of X₁ and Y₁ represents —NR₇—, —O— or —S—;
R₁ represents a substituted or unsubstituted (C6-C30)aryl, or a substituted or unsubstituted (3- to 30-membered)heteroaryl;
R₂ to R₇ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino, or may be linked to an adjacent substituent(s) to form a ring(s);
L₁, U₁, and U₂ each independently represent a single bond, a substituted or unsubstituted (C6-C30)arylene, or a substituted or unsubstituted (3- to 30-membered)heteroarylene;
b and c each independently represent an integer of 1 or 2, d represents an integer of 1 to 4, where if b to d represent an integer of 2 or more, each of R₂ to each of R₄ may be the same as or different from each other; and
in formula 1′, at least one of deuterium is included.

The compound represented by formula 1′ may be selected from the group consisting of the following compounds, but is not limited thereto.

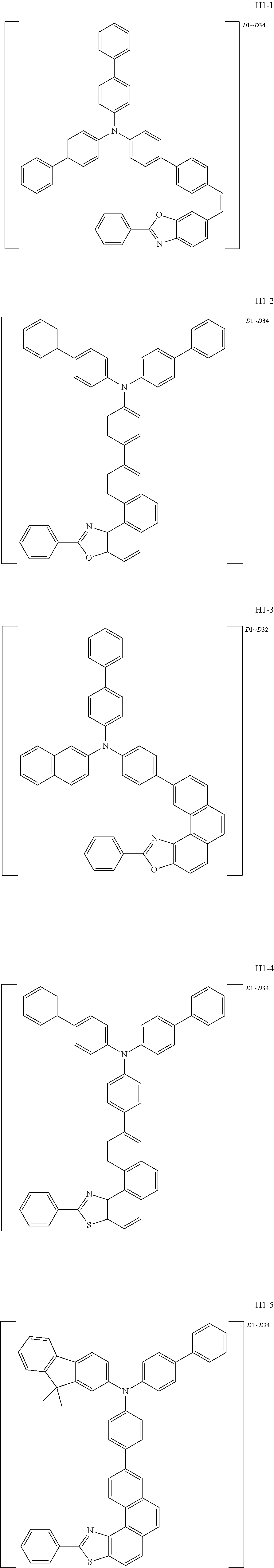

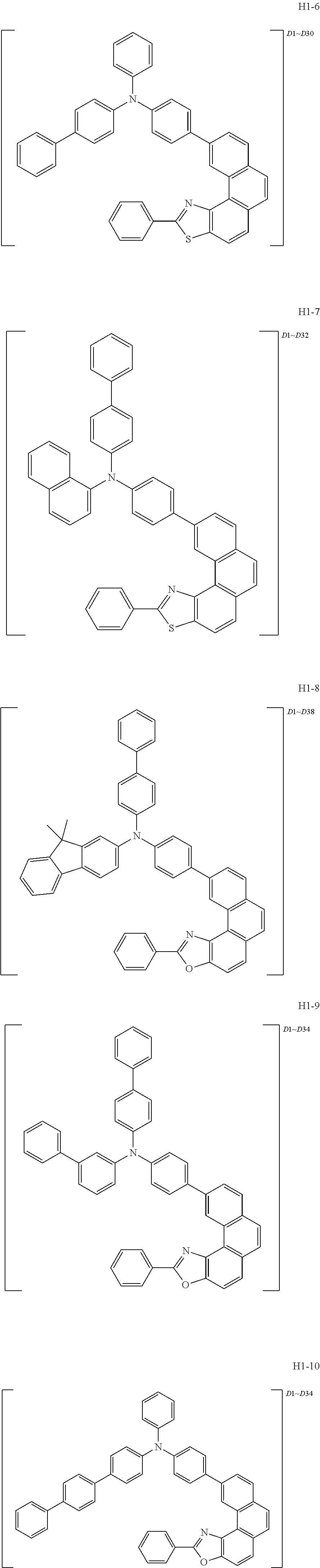

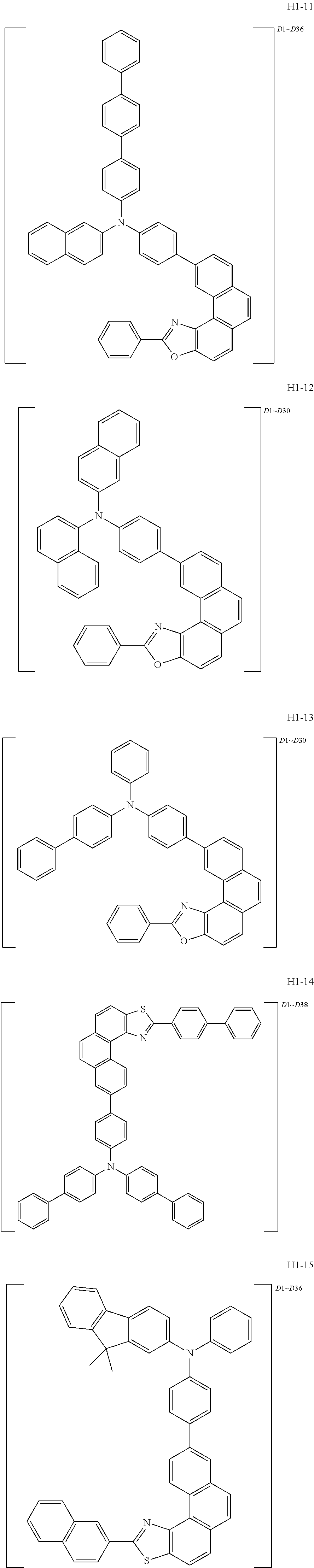

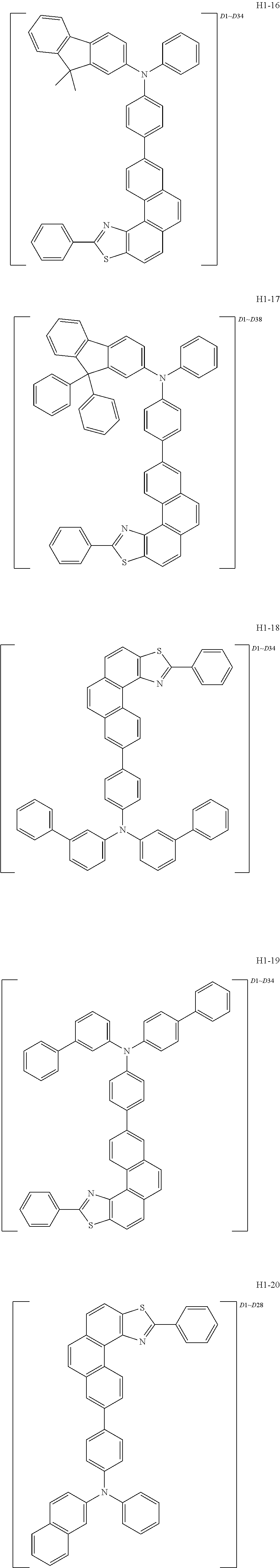

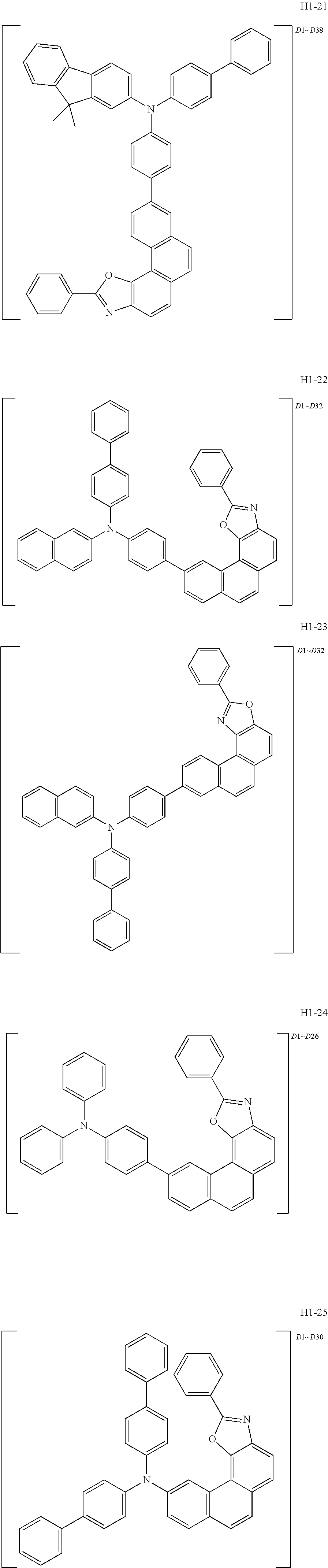

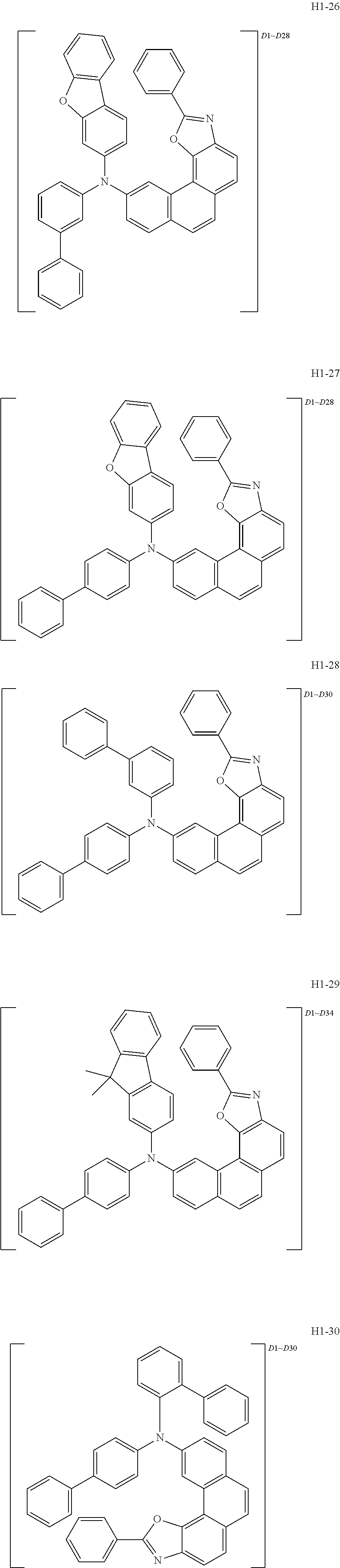

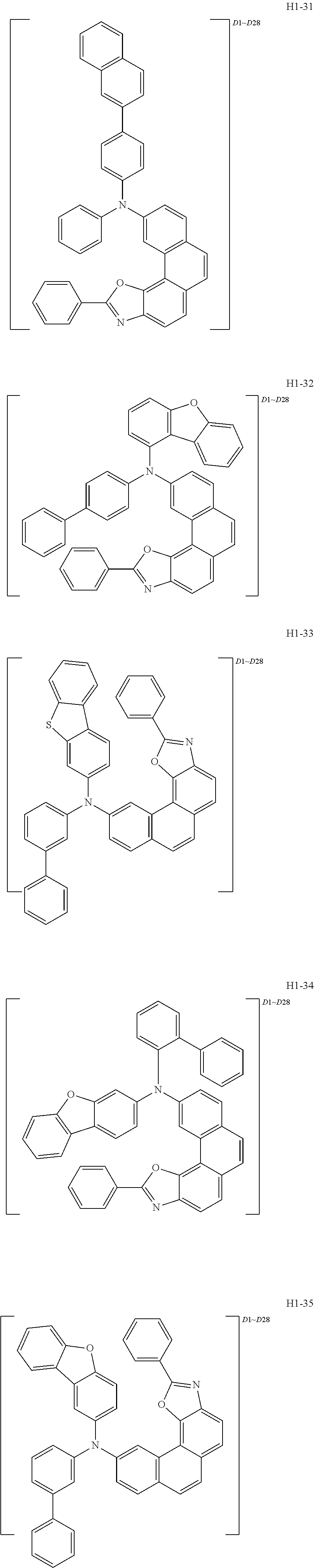

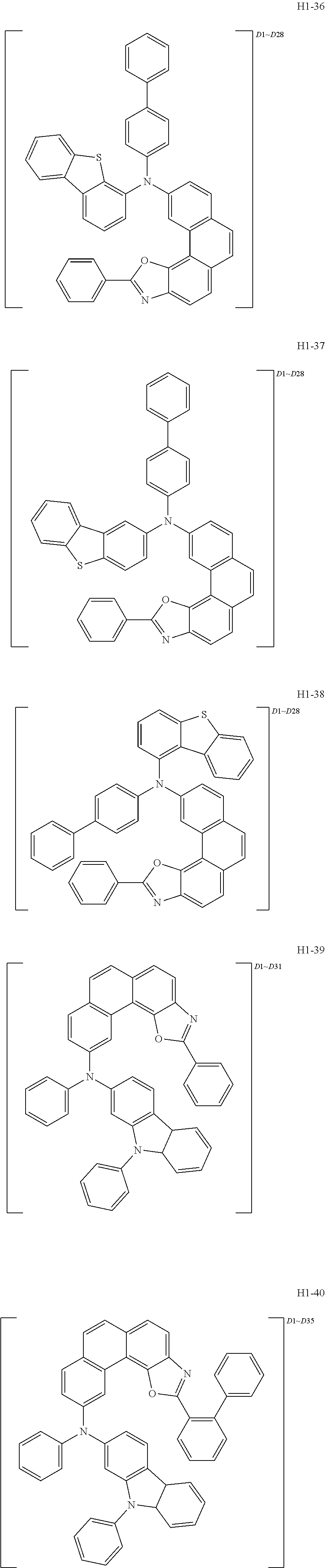

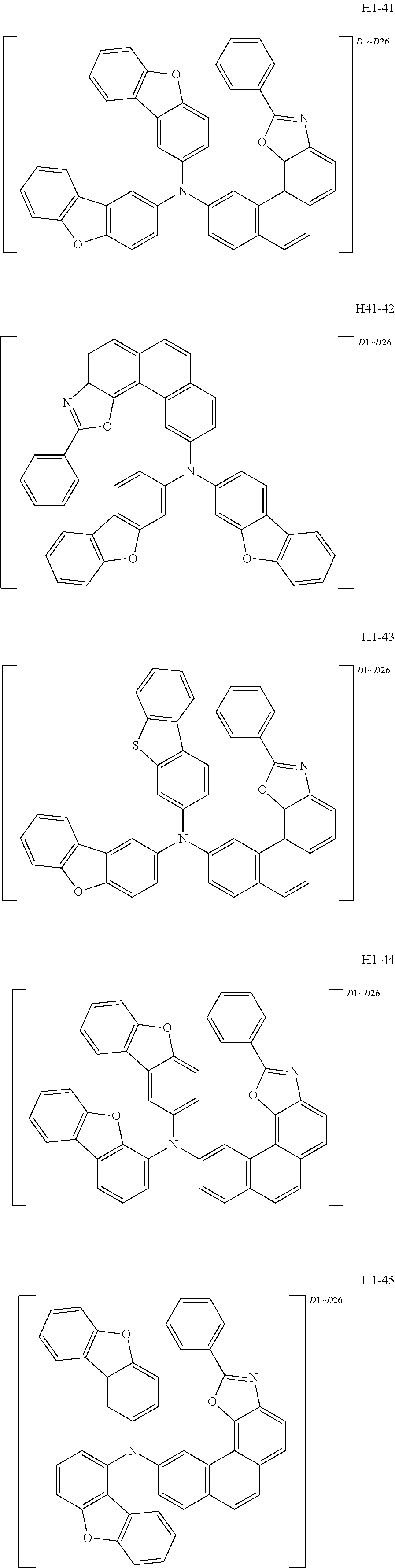

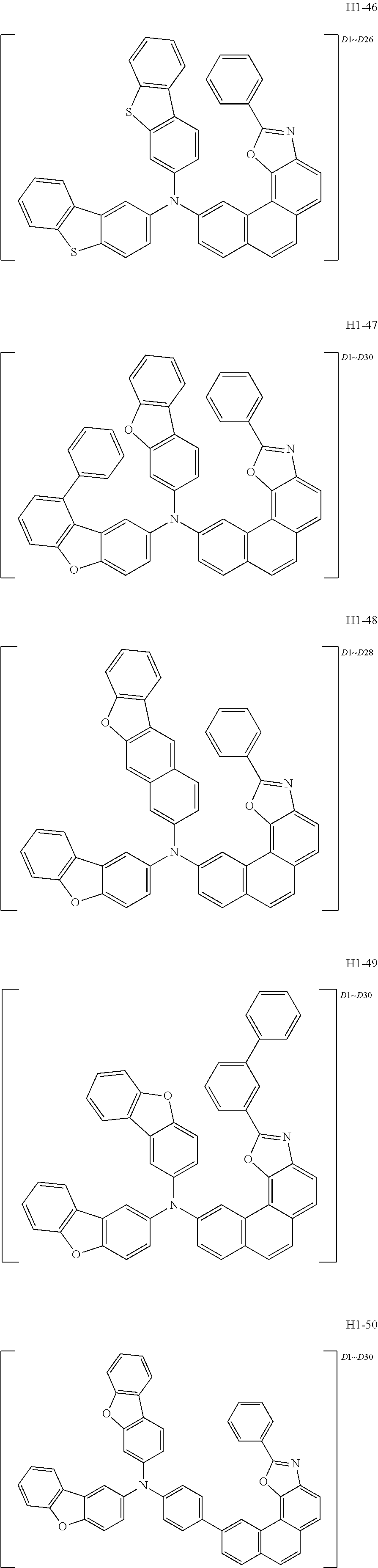

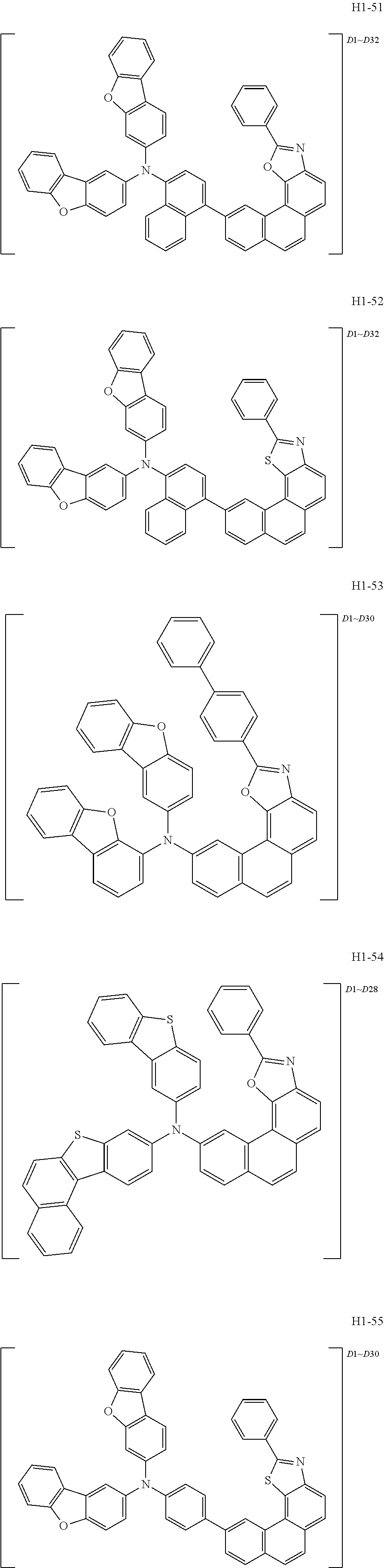

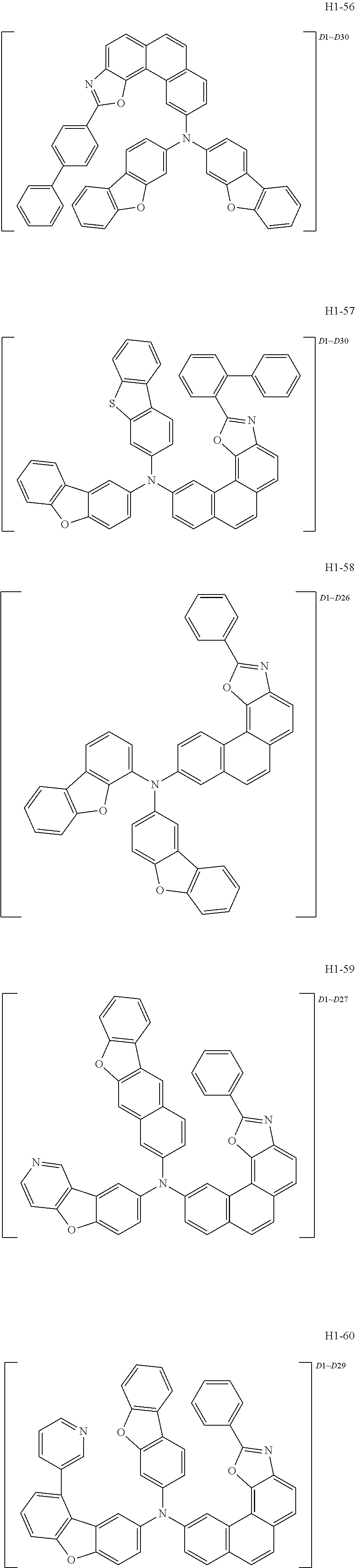

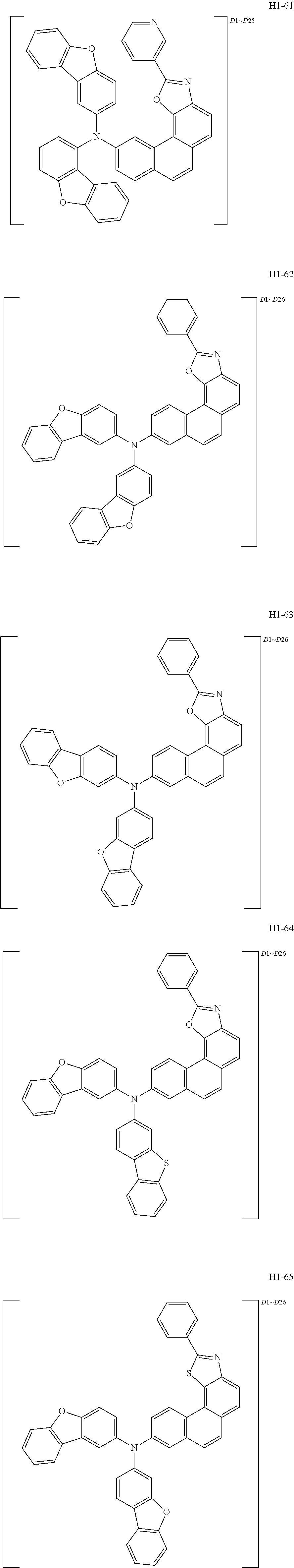

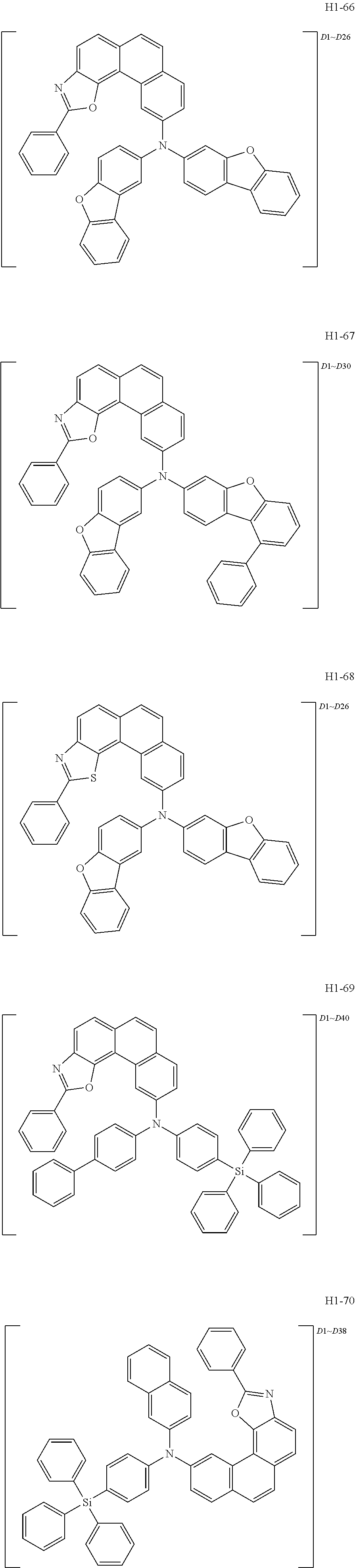

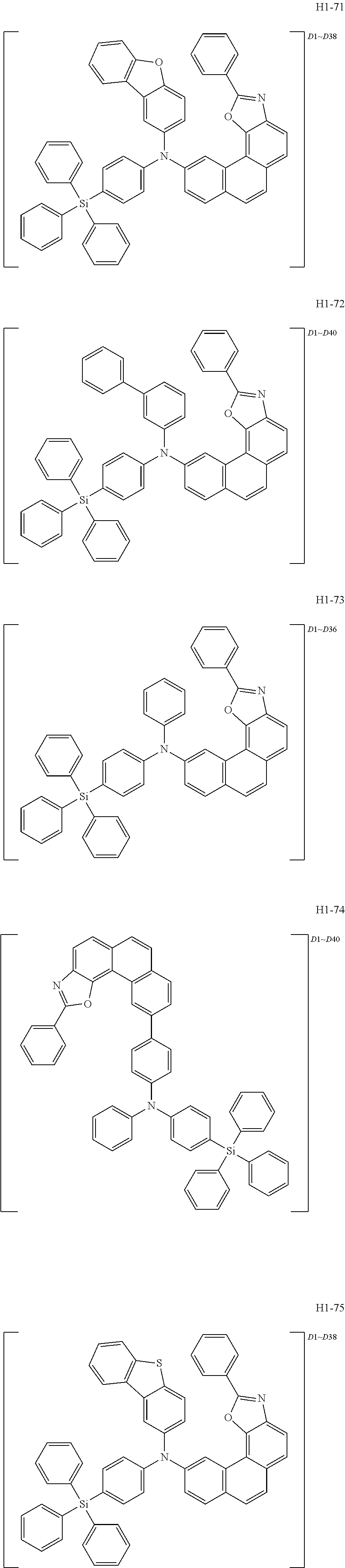

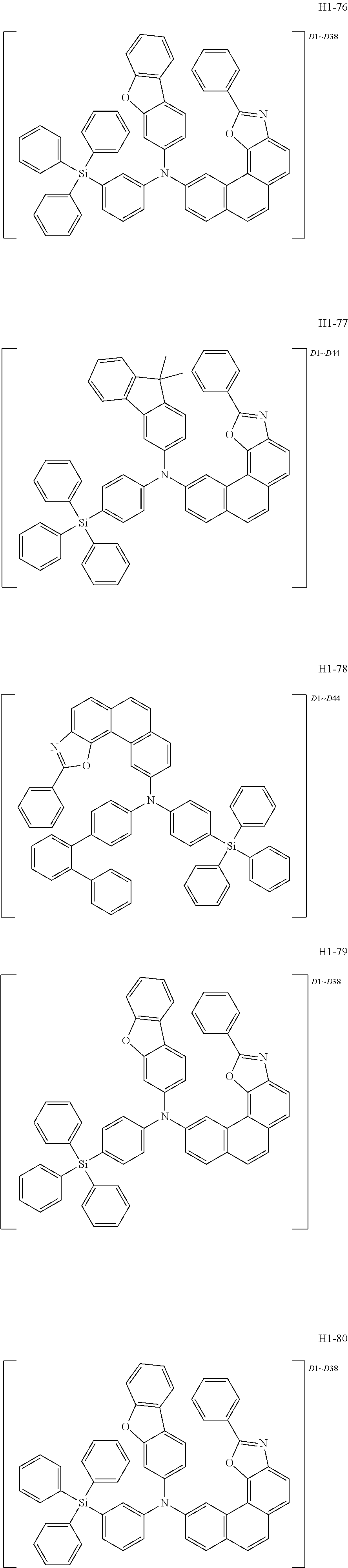

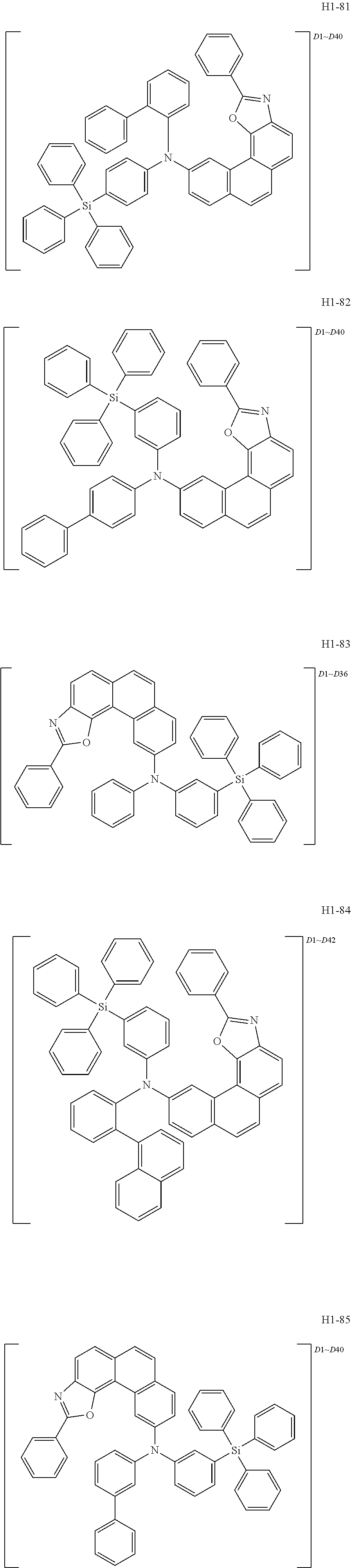

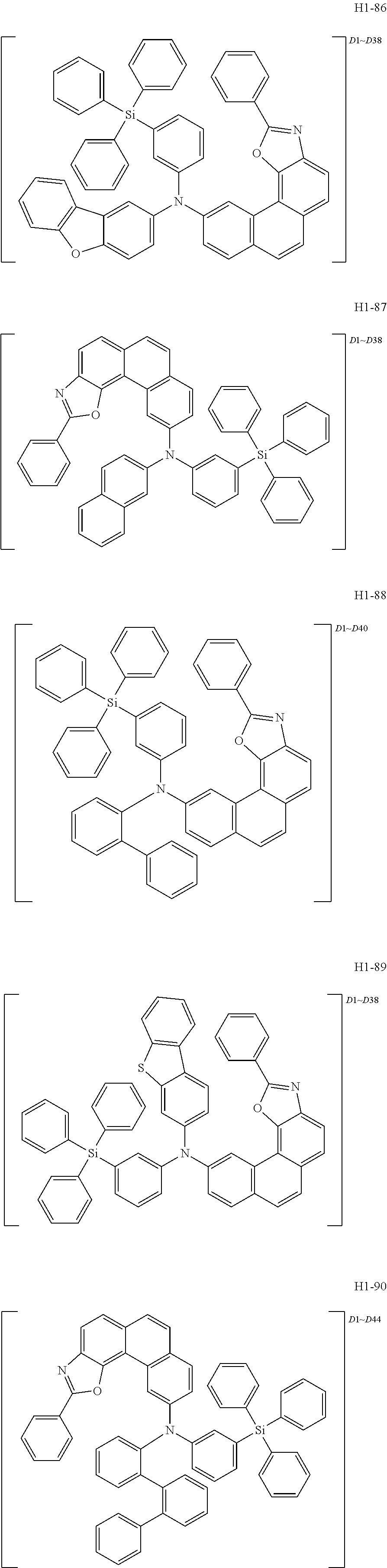

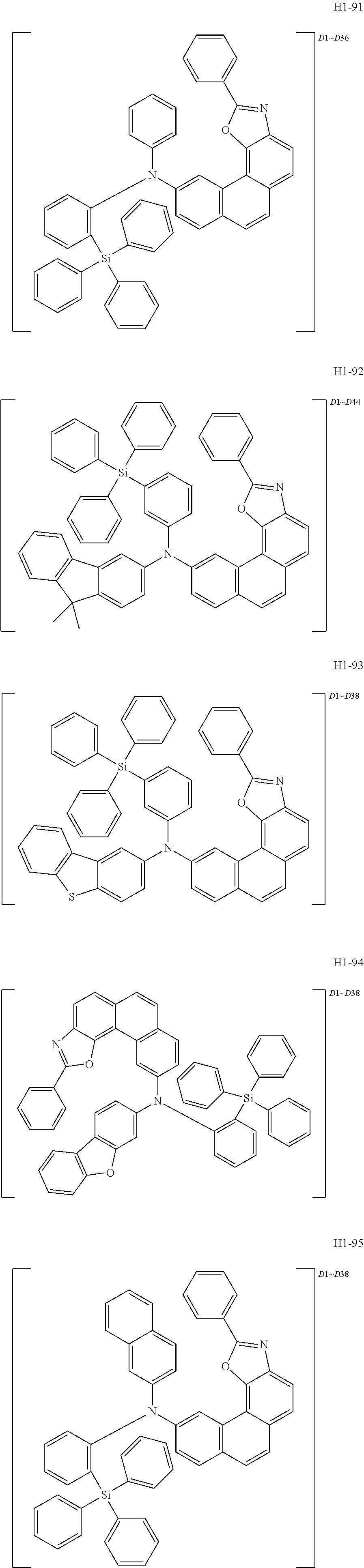

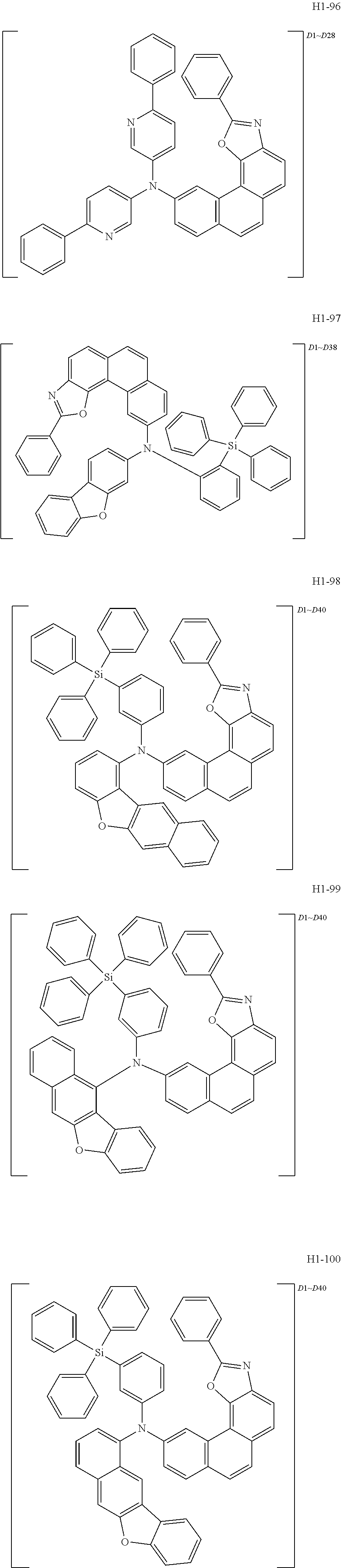

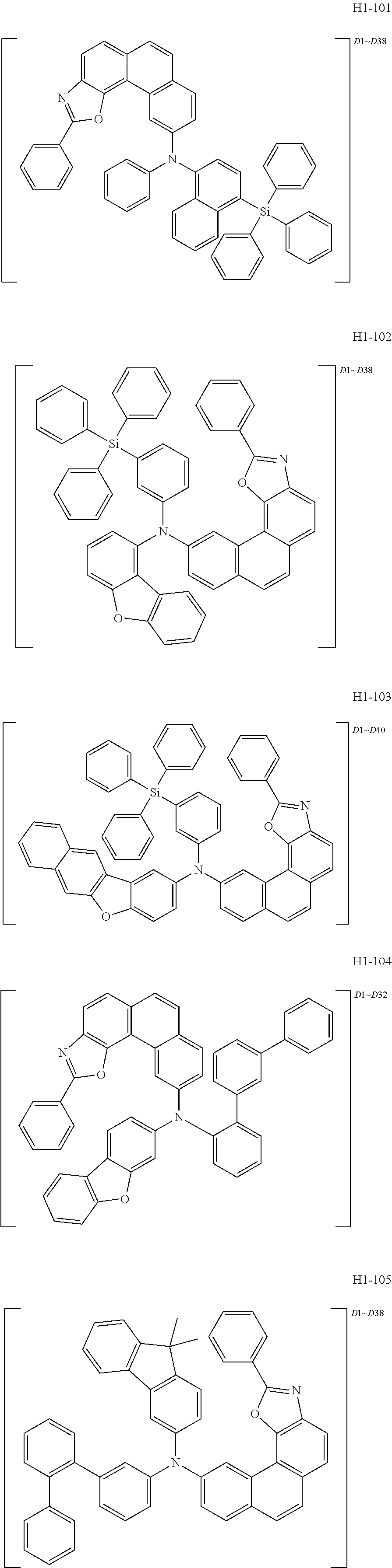

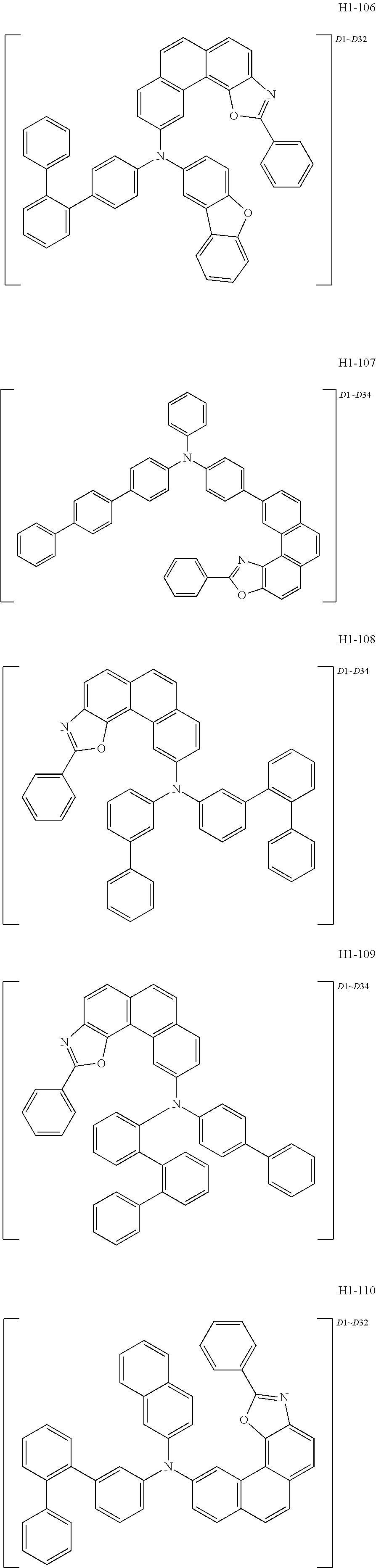

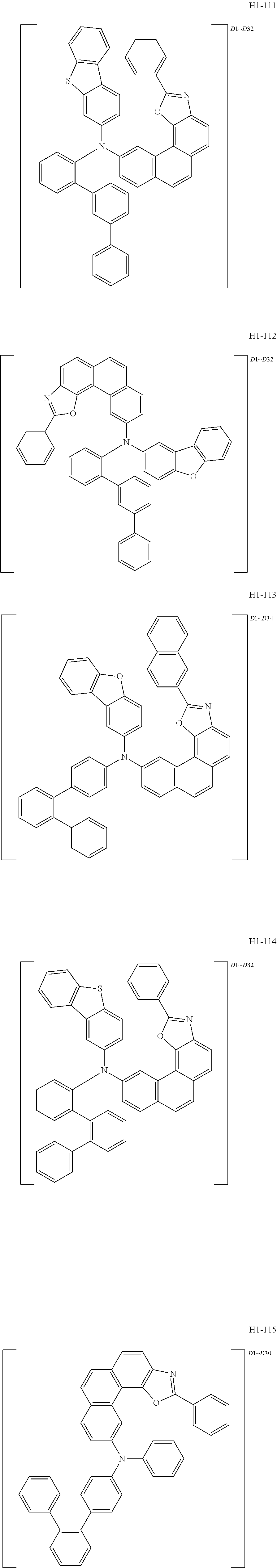

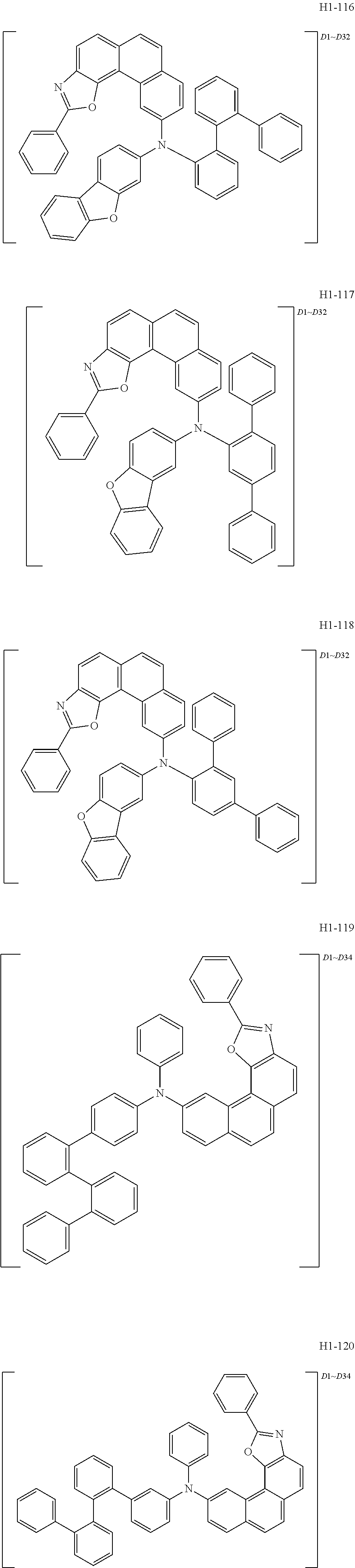

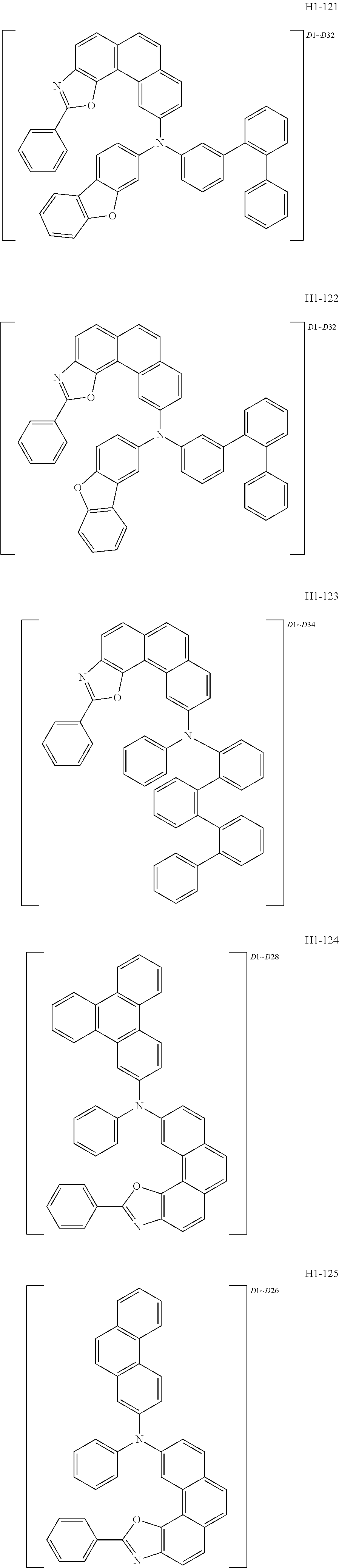

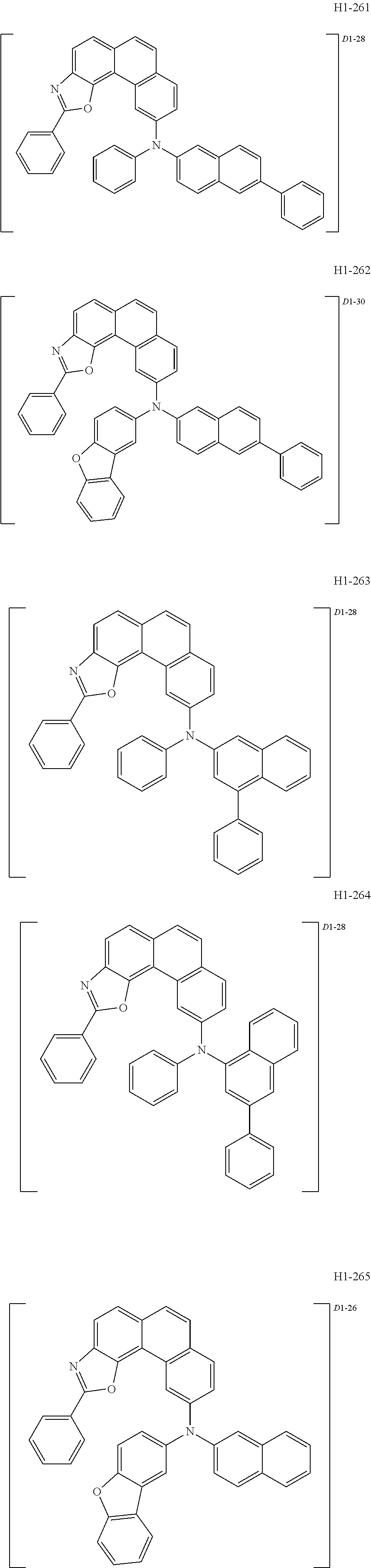

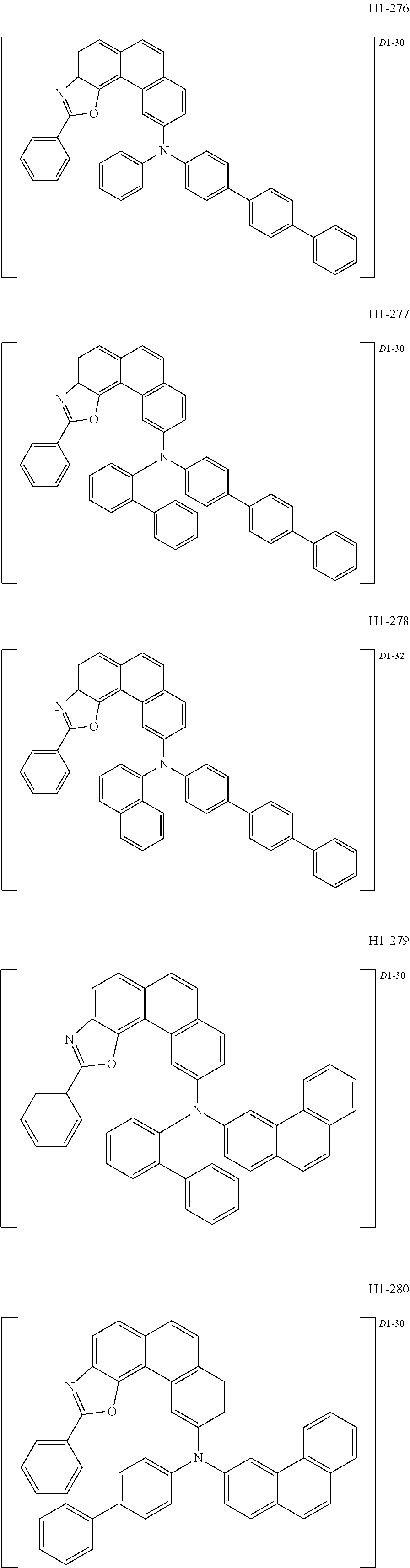

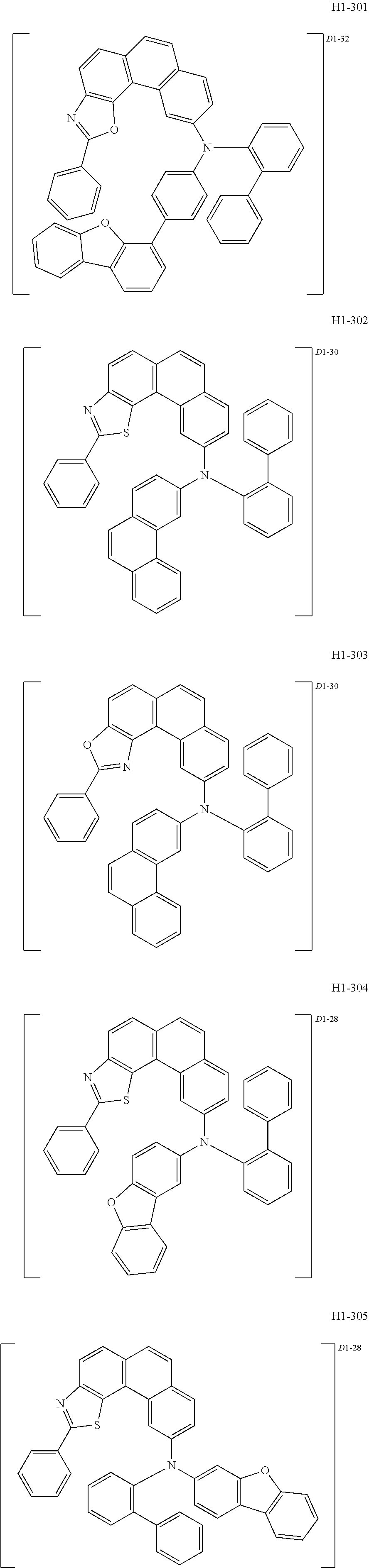

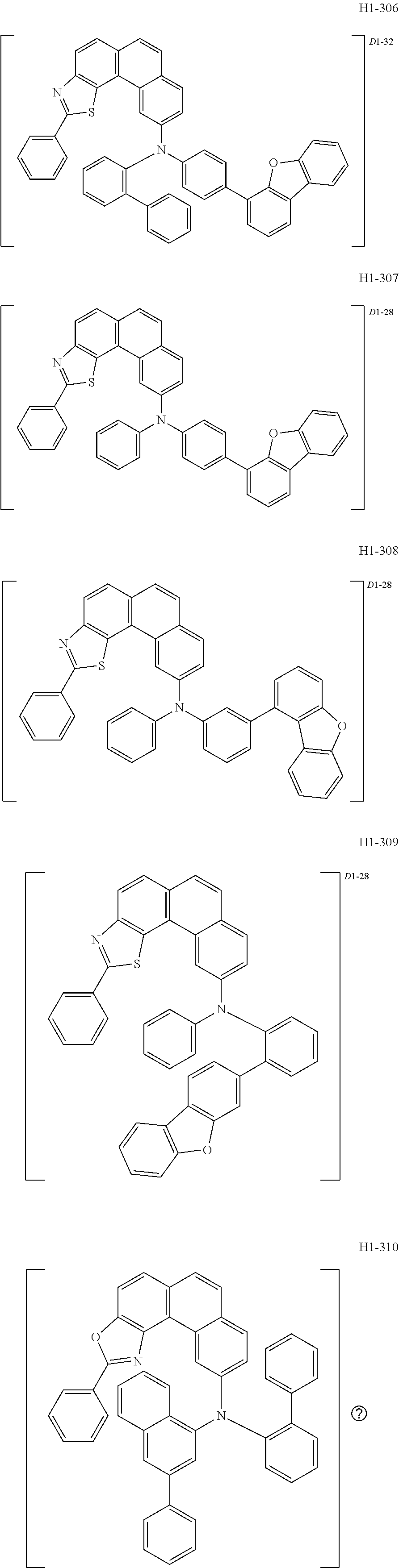

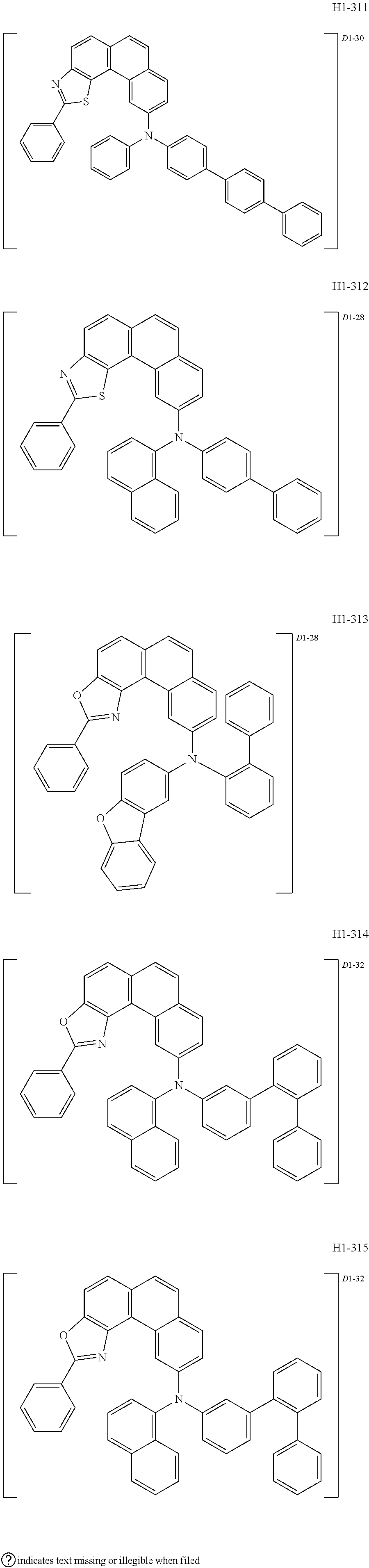

Hereinafter, a plurality of host materials according to the present disclosure will be described.

The plurality of host materials according to the present disclosure comprises a first host material comprising at least one compound represented by the formula 1 above and a second host material comprising at least one compound represented by the formula 2 above, wherein at least one of the first host material and the second host material comprises deuterium. In addition, the plurality of host materials further comprises a third host material, wherein, as an example, the third host material comprises a plurality of host materials comprising the following formula 3.

Hereinafter, the compound represented by formula 1 will be described in more detail.

In formula 1, X₁ and Y₁ each independently represent —N═, —NR₇—, —O— or —S—, with a proviso that one of X₁ and Y₁ represents —N═, the other of X₁ and Y₁ represents —NR₇—, —O— or —S—. According to one embodiment of the present disclosure, one of X₁ and Y₁ represents —N═, and the other of X₁ and Y₁ represents —O— or —S—.

In formula 1, R₁ represents a substituted or unsubstituted (C6-C30)aryl, or a substituted or unsubstituted (3- to 30-membered)heteroaryl. According to one embodiment of the present disclosure, R₁ represents a (C6-C15)aryl unsubstituted or substituted with deuterium, or a (5- to 15-membered)heteroaryl unsubstituted or substituted with deuterium. According to another embodiment of the present disclosure, R₁ represents a (C6-C15)aryl unsubstituted or substituted with deuterium, or a (6- to 13-membered)heteroaryl unsubstituted or substituted with deuterium. Specifically, R₁ may be a phenyl, a biphenyl, a naphthyl, or a pyridyl, etc., which may be further substituted with deuterium.

In formula 1, R₂ to R₇ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino, or may be linked to an adjacent substituent(s) to form a ring(s).

According to one embodiment of the present disclosure, R5 and R6 each independently a substituted or unsubstituted (C6-C28)aryl, a substituted or unsubstituted (6- to 25-membered)heteroaryl. According to another embodiment of the present disclosure, R5 and R6 each independently deuterium, a (C6-C28)aryl unsubstituted or substituted with at least one of deuterium, a (C1-C10)alkyl(s), a (C6-C12)aryl(s) and a tri(C6-C10)arylsillyl, or a (6- to 20-membered)heteroaryl unsubstituted or substituted with at least one of deuterium, a (C6-C10)aryl(s) and a (6- to 10-membered)heteroaryl(s). Specifically, R₂ to R₄ each zindependently may be hydrogen or deuterium; R₅ and R₆ each independently may be a phenyl unsubstituted or substituted with at least one of a naphthyl(s) and a triphenylsilyl(s), a biphenyl, a terphenyl, a quarterphenyl, a naphthyl unsubstituted or substituted with a triphenylsilyl(s), a phenanthrenyl, a triphenylenyl, a dimethylfluorenyl, a diphenylfluorenyl, a pyridyl unsubstituted or substituted with a phenyl(s), a dibenzofuranyl unsubstituted or substituted with at least one of a phenyl(s) and a pyridyl(s), a dibenzothiophenyl, a carbazolyl substituted with a phenyl(s), a benzofuropyridinyl, a benzonaphthofuranyl, a benzonaphthothiophenyl, or a triphenylsilyl, which may be further substituted with deuterium.

According to another embodiment of the present disclosure, R₅ and R₆ independently may be a substituted or unsubstituted phenyl, a substituted or unsubstituted naphthyl, a substituted or unsubstituted biphenyl, a substituted or unsubstituted terphenyl, a substituted or unsubstituted phenanthrenyl, a substituted or unsubstituted fluorenyl, a substituted or unsubstituted benzofluorenyl, a substituted or unsubstituted triphenylenyl, a substituted or unsubstituted spirobifluorenyl, a substituted or unsubstituted pyridiyl, a substituted or unsubstituted triazinyl, a substituted or unsubstituted pyrimidinyl, a substituted or unsubstituted quinolyl, a substituted or unsubstituted quinazolinyl, a substituted or unsubstituted quinoxalinyl, a substituted or unsubstituted benzoquinazolinyl, a substituted or unsubstituted benzoquinoxalinyl, a substituted or unsubstituted benzofuropyridinyl, a substituted or unsubstituted benzofuropyrimidinyl, a substituted or unsubstituted carbazolyl, a substituted or unsubstituted dibenzothiophenyl, a substituted or unsubstituted benzothiophenyl, substituted or unsubstituted dibenzofuranyl, substituted or unsubstituted benzofuranyl, substituted or unsubstituted naphthyridinyl, substituted or unsubstituted benzonaphthofuranyl, a substituted or unsubstituted benzonaphthothiophenyl, or a triphenylsilyl.

L₁, U₁, and U₂ each independently represent a single bond, a substituted or unsubstituted (C6-C30)arylene, or a substituted or unsubstituted (3- to 30-membered)heteroarylene. According to one embodiment of the present disclosure, L₁, U₁, and U₂ each independently represent a single bond, a substituted or unsubstituted (C6-C25)arylene, or a substituted or unsubstituted (3- to 25-membered)heteroarylene. According to another embodiment of the present disclosure, L₁, U₁, and U₂ each independently represent a single bond, a (C6-C15)arylene unsubstituted or substituted with deuterium, or a (3- to 25-membered)heteroarylene unsubstituted or substituted with deuterium. Specifically, L₁, U₁, and U₂ each independently represent a single bond, a phenylene, a biphenylene, a terphenylene, a naphthylene, a dibenzofuranylene, a pyridylene, a carbazolylene, etc., which may be further substituted with deuterium.

In formula 1, b and c each independently represent an integer of 1 or 2, d represents an integer of 1 to 4, where if b to d represent an integer of 2 or more, each of R₁ to each of R₄ may be the same as or different from each other.

According to one embodiment of the present disclosure, the formula 1 may be represented by at least one of the following formulas 1-1 to 1-4.

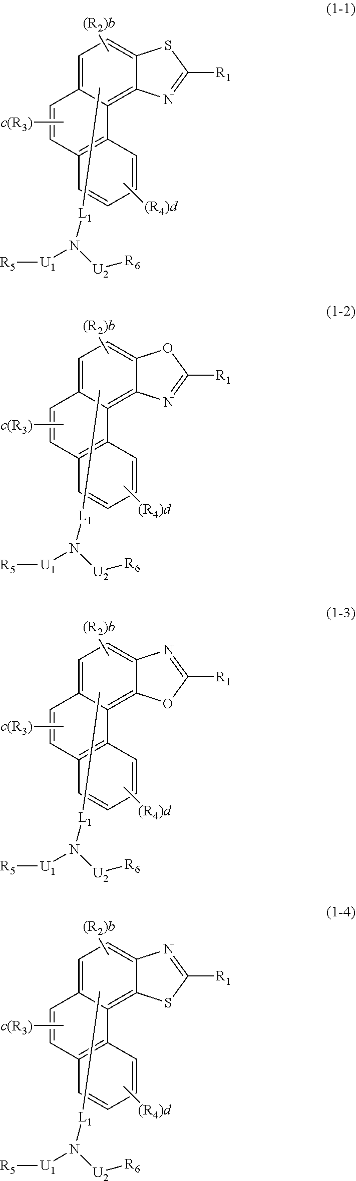

In formulas 1-1 to 1-4, R₁ to R₆, L₁, U₁, U₂, and b to d are as defined in formula 1.

According to one embodiment of the present disclosure, the deuterium substitution rate when the compound represented by formula 1 contains deuterium may be about 0.1% to 100%, according to one embodiment about 10% to about 95%, according to another embodiment about 20% to about 90%, according to another embodiment about 30% to about 85%, according to another embodiment about 40% to about 80%, according to another embodiment about 50% to about 75%. The compound of formula 1 substituted with the deuterium substitution rate may increase the stability of the compound, by increasing bond dissociation energy due to deuteration, and an organic electroluminescent device comprising the compound device may exhibit improved lifetime properties.

Hereinafter, the compound represented by formula 2 will be described in more detail.

In formula 2, X represents O or S.

In formula 2, HAr represents a substituted or unsubstituted (3- to 30-membered)heteroaryl, including at least one nitrogen atom. According to one embodiment of the present disclosure, HAr represents a substituted or unsubstituted (6- to 15-membered)heteroaryl, including at least two nitrogen atoms. According to another embodiment of the present disclosure, HAr represents a substituted or unsubstituted (6-membered)heteroaryl, including at least two nitrogen atoms, and the substituent of the heteroaryl is deuterium; a (C6-C20)aryl unsubstituted or substituted with at least one of deuterium and a (C6-C12)aryl(s); or a (6- to 15-membered) heteroaryl unsubstituted or substituted with deuterium. Specifically, HAr is a substituted triazinyl, and the substituents of the triazinyl are at least one of a phenyl, a biphenyl, a terphenyl, a phenylnaphthyl, a naphthylphenyl, a naphthyl substituted with a biphenyl(s), a phenanthrenyl, a benzo[c]phenanthrenyl, a chrysenyl and a dibenzofuranyl, which may be further substituted with deuterium.

In formula 2, L represents a single bond, a substituted or unsubstituted (C6-C30)arylene, or a substituted or unsubstituted (3- to 30-membered)heteroarylene. Specifically, L may be a single bond.

In formula 2, R₃ and R₉ each independently represent hydrogen, deuterium, a substituted or unsubstituted (C6-C30)aryl, a substituted or unsubstituted (3- to 30-membered)heteroaryl, or —SiR₂₁R₂₂R₂₃. According to one embodiment of the present disclosure, R₃ and R₉ each independently represent hydrogen, deuterium, or a substituted or unsubstituted (C6-C20)aryl. According to another embodiment of the present disclosure, R₃ and R₉ each independently represent hydrogen, deuterium, or a (C6-C15)aryl unsubstituted or substituted with at least one of deuterium and a (C6-C12)aryl(s). Specifically, R₃ and R₉ each independently may be hydrogen, deuterium, a phenyl, a biphenyl, a naphthyl, a phenylnaphthyl, a naphthylphenyl, or a phenanthrenyl, which may be further substituted with deuterium.

R₂₁ to R₂₃ each independently represent a substituted or unsubstituted (C1-C30)alkyl, a substituted or unsubstituted (C6-C30)aryl, or a substituted or unsubstituted (3- to 30-membered)heteroaryl.

In formula 2, e represents an integer of 1 to 4, f represents an integer of 1 to 3, where if e and f represent an integer of 2 or more, each of R₃ and each of R₉ may be the same as or different from each other.

According to one embodiment of the present disclosure, the formula 2 may be represented by the following formula 2-1.

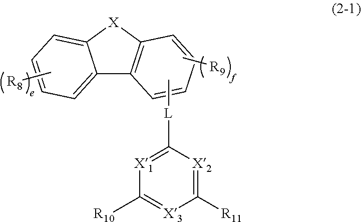

In formula 2-1, X′₁ to X′₃ each independently represent CR′ or N, with a proviso that at least two of X′₁ to X′₃ represent N. Specifically, all of X′₁ to X′₃ may be N.

R′ represents hydrogen or deuterium.

In formula 2-1, R₁₀ and R₁₁ each independently represent a substituted or unsubstituted (C6-C30)aryl, or a substituted or unsubstituted (3- to 30-membered)heteroaryl. According to one embodiment of the present disclosure, R₁₀ and R₁₁ each independently represent a substituted or unsubstituted (C6-C25)aryl, or a substituted or unsubstituted (6- to 20-membered)heteroaryl. According to another embodiment of the present disclosure, R₁₀ and R₁₁ each independently represent a (C6-C20)aryl unsubstituted or substituted with at least one of deuterium and a (C6-C18)aryl(s); or a (6- to 15-membered)heteroaryl unsubstituted or substituted with at least one of deuterium and a (C6-C18)aryl(s). Specifically, R₁₀ and R₁₁ each independently may be a phenyl unsubstituted or substituted with a naphthyl substituted with a phenyl(s) or a phenanthrenyl(s), a biphenyl, a terphenyl, a quarterphenyl, a phenylnaphthyl, a naphthylphenyl, a naphthyl unsubstituted or substituted with a biphenyl(s) or a naphthyl(s), a phenanthrenyl unsubstituted or substituted with a phenyl(s) or a naphthyl(s), a benzo[c]phenanthrenyl, a chrysenyl, a triphenylene, a fluoranthenyl, or a dibenzofuranyl unsubstituted or substituted with a phenyl(s), which may be further substituted with deuterium.

In formula 2-1, X, L, R₈, R₉, e and f are as defined in formula 2.

According to one embodiment of the present disclosure, the formula 2 may be represented by at least one of the following formulas 2-1-1 to 2-1-4.

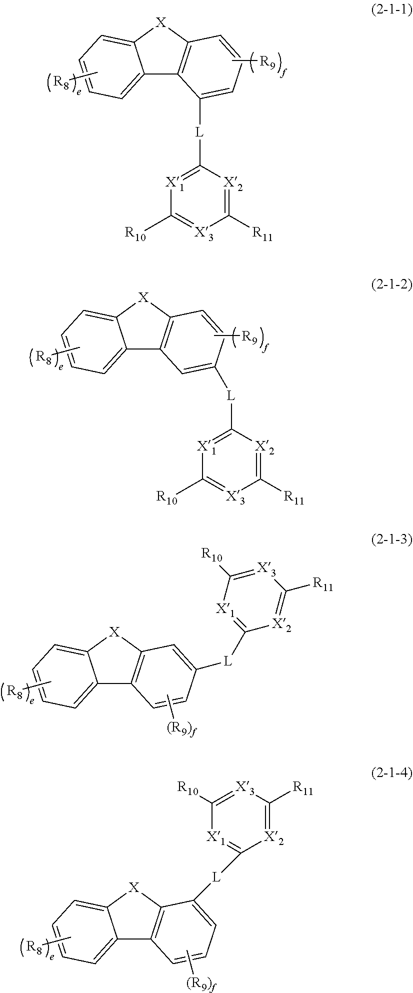

In formulas 2-1-1 to 2-1-4,
X, X′₁ to X′₃, R₈ to R₁₁, L, e and f are as defined in formula 2-1.

According to one embodiment of the present disclosure, the deuterium substitution rate when the compound represented by formula 2 contains deuterium may be about 0.1% to 100%, according to one embodiment about 10% to about 95%, according to another embodiment about 20% to about 90%, according to another embodiment about 30% to about 85%, according to another embodiment about 40% to about 80%, according to another embodiment about 50% to about 75%. The compound of formula 2 substituted with the deuterium substitution rate may increase the stability of the compound, by increasing bond dissociation energy due to deuteration, and an organic electroluminescent device comprising the compound device may exhibit improved lifetime properties.

Hereinafter, the compound represented by formula 3 will be described in more detail.

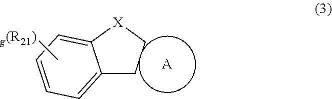

in formula 3,
X represents O, S, CR₃₁R₃₂, NR₃₃, or Se. According to one embodiment of the present disclosure, X represents O, S, CR₃₁R₃₂, or NR₃₃. Specifically, X may be O or S.
R₃₁ to R₃₃ each independently represent hydrogen, deuterium, halogen, a substituted or unsubstituted (C1-C30)alkyl, a substituted or unsubstituted (C6-C30)aryl, or a substituted or unsubstituted (3- to 30-membered)heteroaryl; or may be linked to an adjacent substituent(s) to form a ring(s). According to one embodiment of the present disclosure, R₃₁ to R₃₃ each independently represent hydrogen, deuterium, halogen, a substituted or unsubstituted (C3-C12)alkyl, or a substituted or unsubstituted (C6-C15)aryl. According to another embodiment of the present disclosure, R₃₁ to R₃₃ each independently represent hydrogen, deuterium, halogen, a substituted or unsubstituted (C3-C12)alkyl, or a substituted or unsubstituted (C6-C15)aryl. Specifically, R₃₁ to R₃₃ each independently may be hydrogen or deuterium.

The A represents a substituted or unsubstituted phenanthrene ring represented by the following formula 3-1.

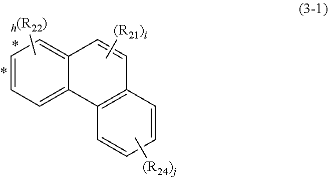

In formulas 3 and 3-1,
R₂₁ to R₂₄ each independently represent hydrogen, deuterium, a halogen, a substituted or unsubstituted (C1-C30)alkyl, a substituted or unsubstituted (C6-C30)aryl,

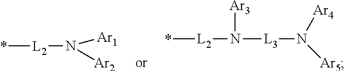

with a proviso that at least one of R₂₁ to R₂₄ represents

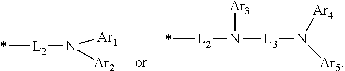

According to one embodiment of the present disclosure, R₂₁ to R₂₄ each independently represent hydrogen, deuterium, a halogen, a substituted or unsubstituted (C1-C15)alkyl a substituted or unsubstituted (C6-C15)aryl,

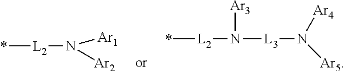

According to another embodiment of the present disclosure, R₂₁ to R₂₄ each independently represent hydrogen, deuterium, a halogen, an unsubstituted (C1-C15)alkyl, an unsubstituted (C6-C15)aryl,

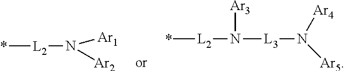

Specifically, R₂₁ to R₂₄ each independently may be hydrogen, deuterium, a phenyl, a biphenyl, a naphthyl,

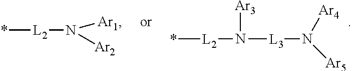

L₂ and L₃ each independently represent a single bond, a substituted or unsubstituted (C6-C30)arylene, a substituted or unsubstituted (C3-C30)cycloalkylene, or a substituted or unsubstituted (3- to 30-membered)heteroarylene. According to one embodiment of the present disclosure, L₂ and L₃ each independently represent a single bond, a substituted or unsubstituted (C6-C12)arylene, or a substituted or unsubstituted (C6-C25)cycloalkylene. Specifically, L₂ and L₃ each independently may be a single bond, a phenylene, a phenylene substituted with a diphenylamino(s), a phenylene substituted with a naphthyl(s), or a naphthylene substituted with a phenyl(s).
Ar₁ to Ar₅ each independently represent a substituted or unsubstituted (C6-C30)aryl or a substituted or unsubstituted (3- to 30-membered)heteroaryl. According to one embodiment of the present disclosure, Ar₁ to Ar₅ each independently represent a (C6-C12)aryl unsubstituted or substituted with at least one of deuterium, a (C5-C15)aryl(s), and a (3- to 30-membered)heteroaryl(s); a (C6-C25)aryl unsubstituted or substituted with a (C6-C18)aryl(s), a (5- to 15-membered)heteroaryl(s), or a (C1-C6)arylene(s); a (5- to 25-membered)heteroaryl unsubstituted or substituted with deuterium or a (C6-C18)aryl(s), a terphenyl, or a quaterphenyl. Specifically, Ar₁ to Ar₅ each independently may be a phenyl unsubstituted or substituted with at least one of deuterium, a naphthyl(s), a carbazolyl(s), a phenylcarbazolyl(s), a dibenzofuranyl(s), a dibenzothiophenyl(s), a phenanthrenyl(s), a fluorenyl(s) substituted with a methyl(s), a pyridinyl(s), and an adamantly; a biphenyl unsubstituted or substituted with deuterium; a phenanthrenyl substituted with a tetramethyl(s), a propendiyldibenzene, a carbazolyl unsubstituted or substituted with a phenyl(s), a chrysenyl, a dibenzofuranyl unsubstituted or substituted with deuterium or a phenyl(s), a dibenzoselenophenyl, a dimethylfluorenyl, a benzonaphthoselenophenyl, a diphenylfluorenyl, a phenanthrenyl substituted or unsubstituted with a phenyl(s), a biphenyl(s) or a pyridine(s); a pyridyl unsubstituted or substituted with a phenyl(s) or a naphthyl(s); a triphenylsilyl, a dibenzothiophenyl unsubstituted or substituted with a phenyl(s), a terphenyl unsubstituted or substituted with a cyano(s), a quaterphenyl, and a phenanthrolinyl substituted with a phenyl(s).
g and j represent an integer of 1 to 4, h and i represent an integer of 1 or 2, where if g to h represent an integer of 2 or more, each of R₂₁ and each of R₂₄ may be the same as or different from each other.
in formula 3-1 represents a site linked to formula 3, and* in R₂₁ to R₂₄ represents a site linked to formula 3 or 3-1.

According to one embodiment of the present disclosure, the formula 3 may be represented by the following formulas 3-2 or 3-3.

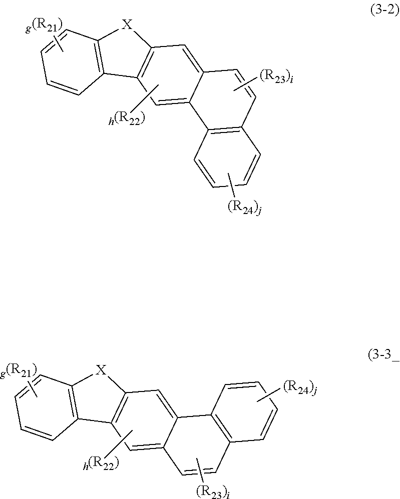

In formulas 3-2 and 3-3, X, R₂₁ to R₂₄, and g to j are as defined in formula 3.

The compound represented by formula 1 may be selected from the group consisting of the following compounds, but is not limited thereto.

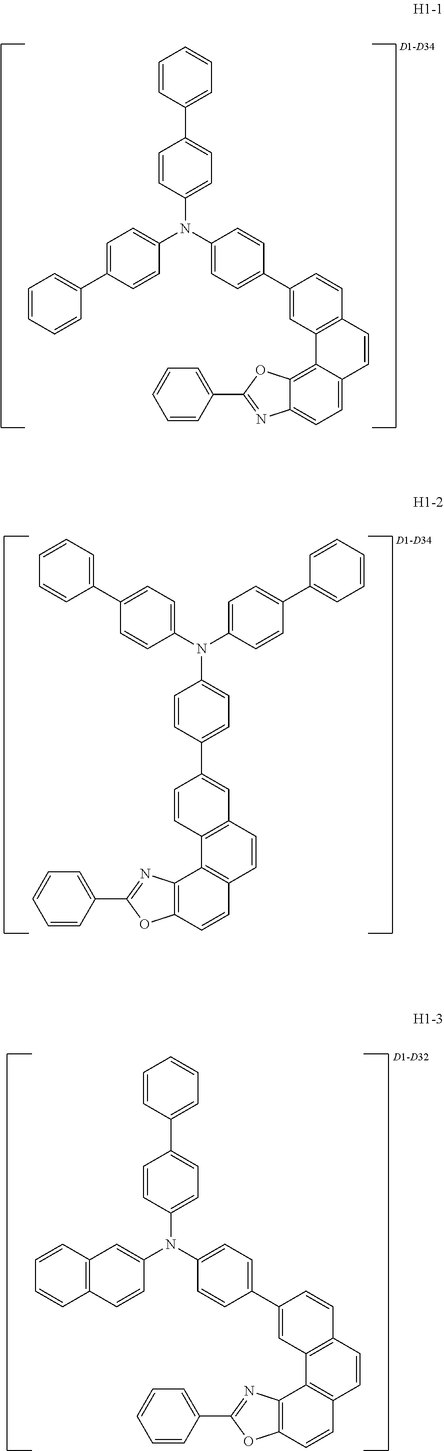

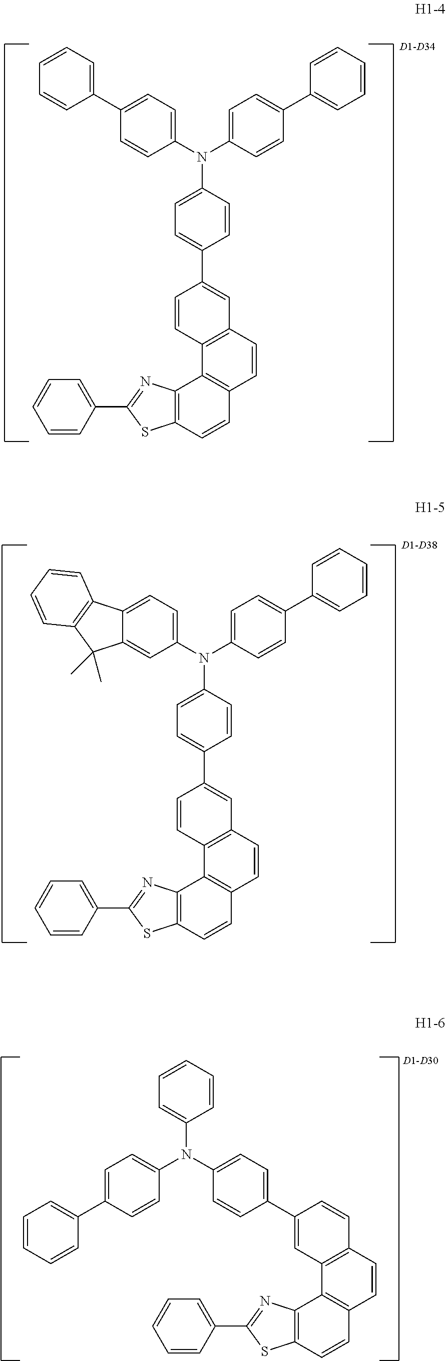

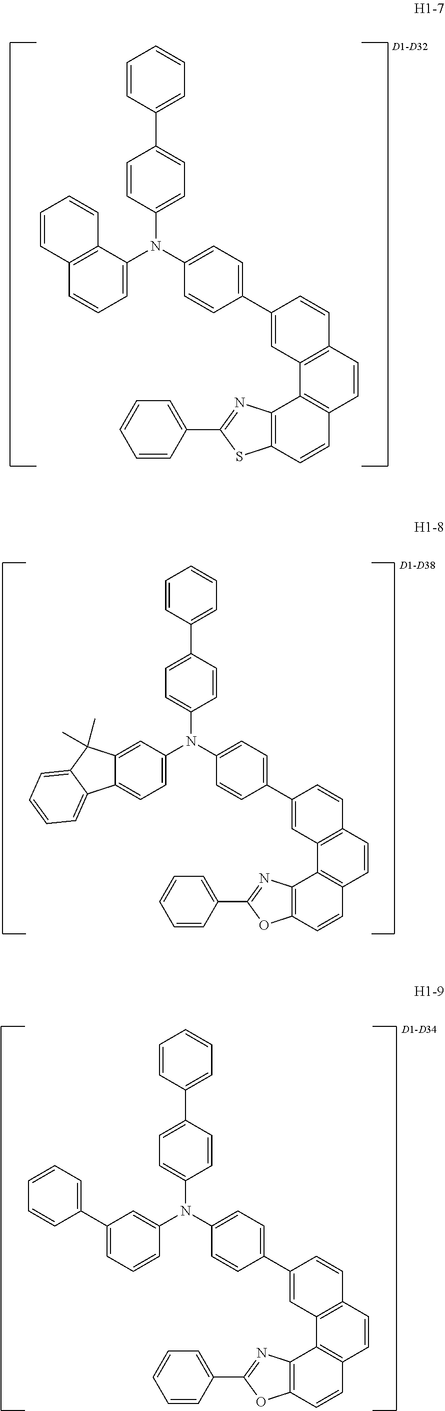

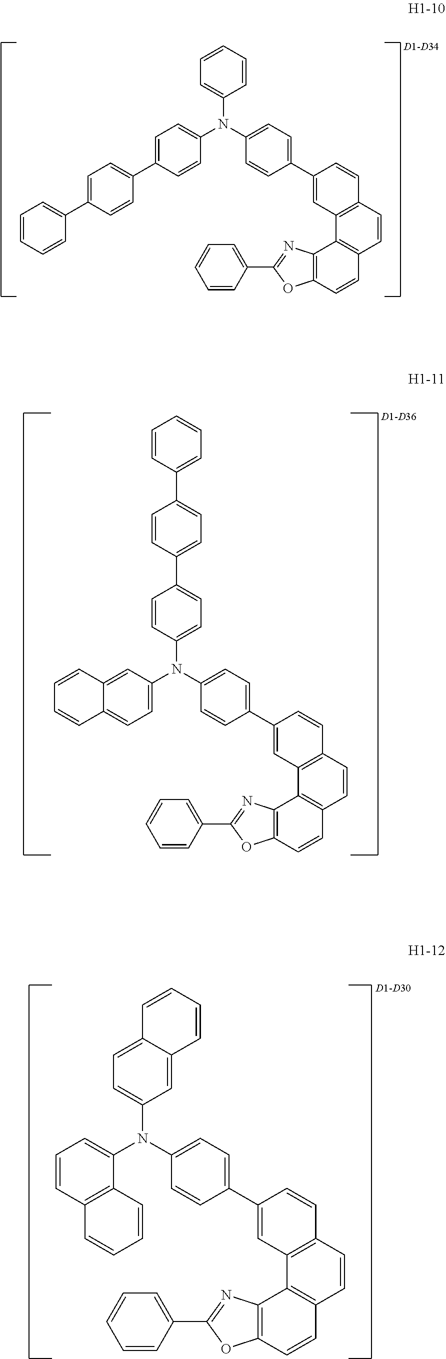

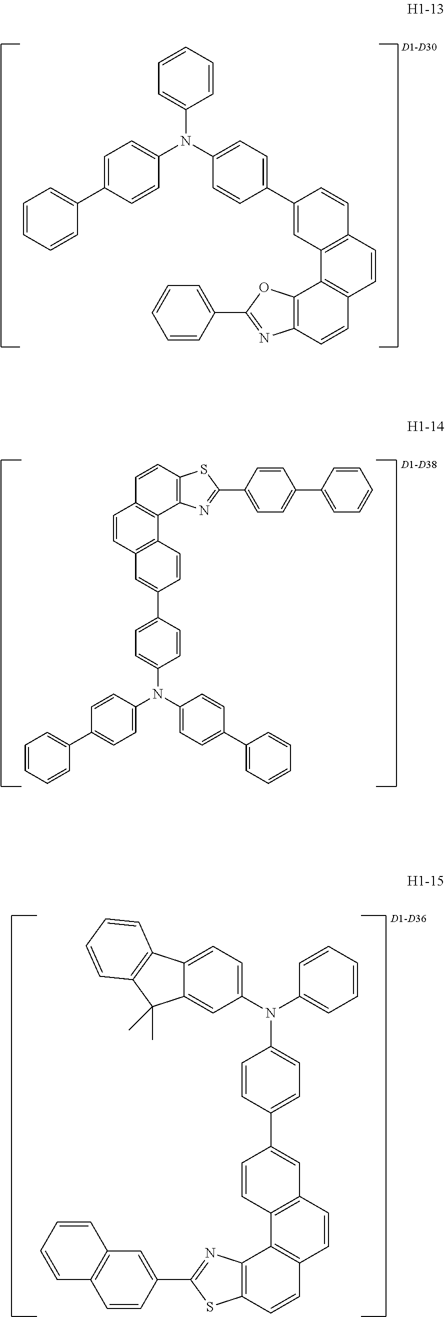

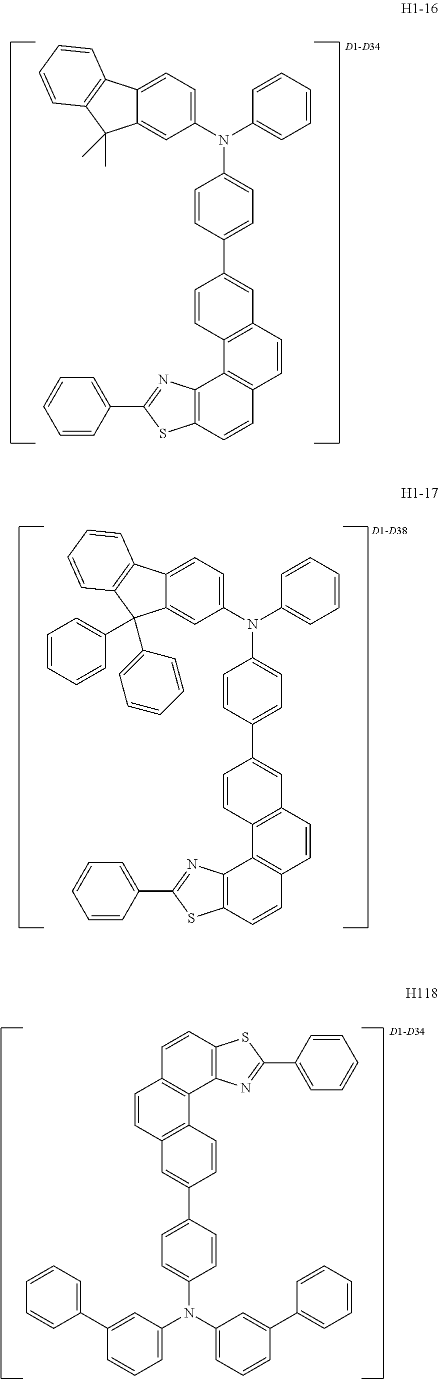

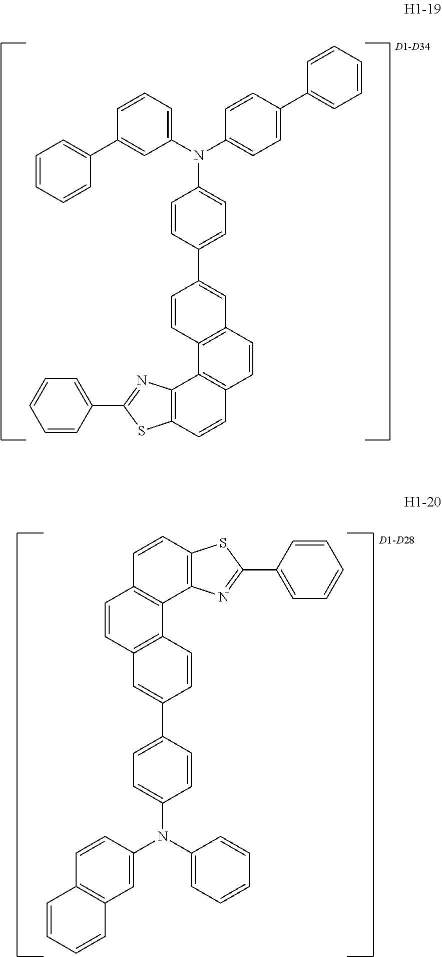

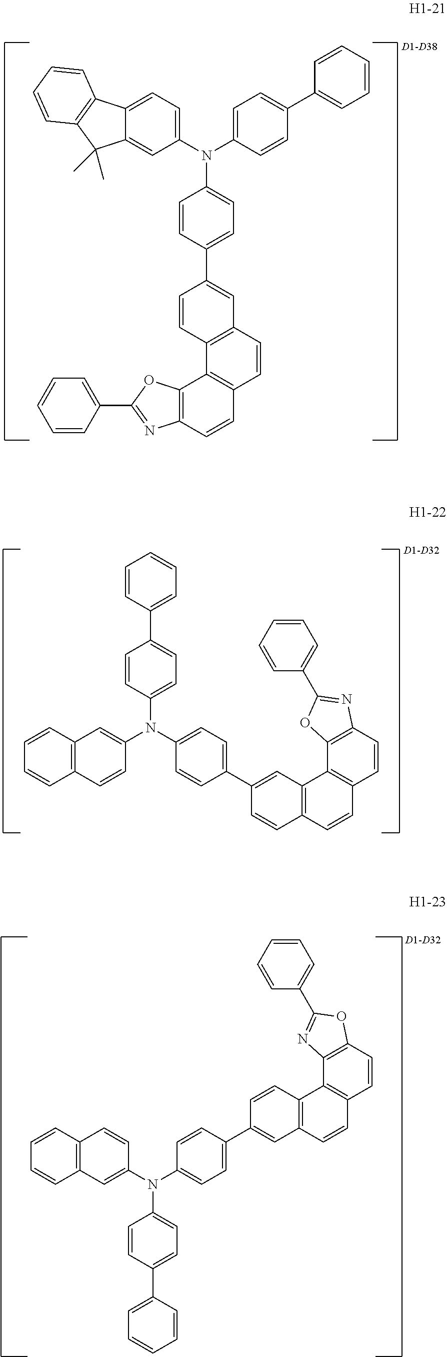

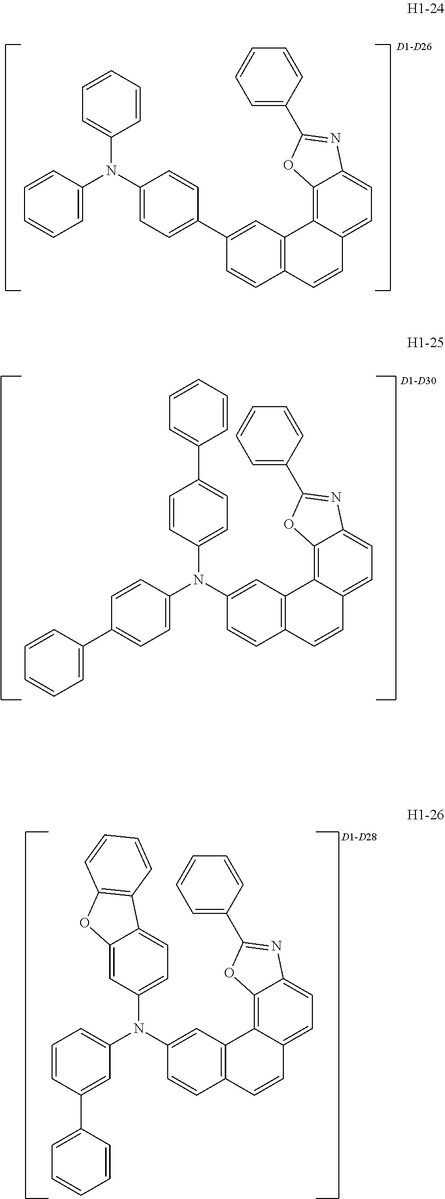

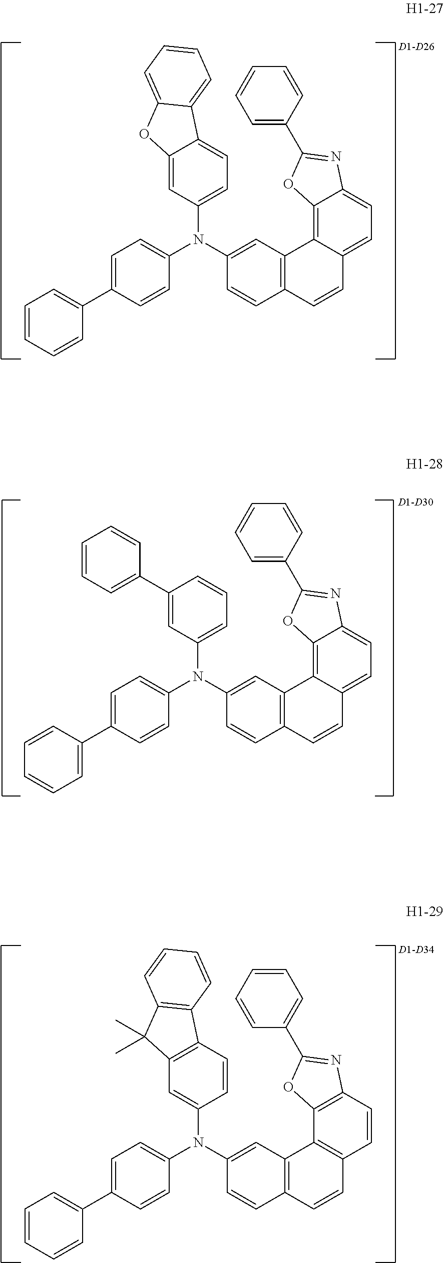

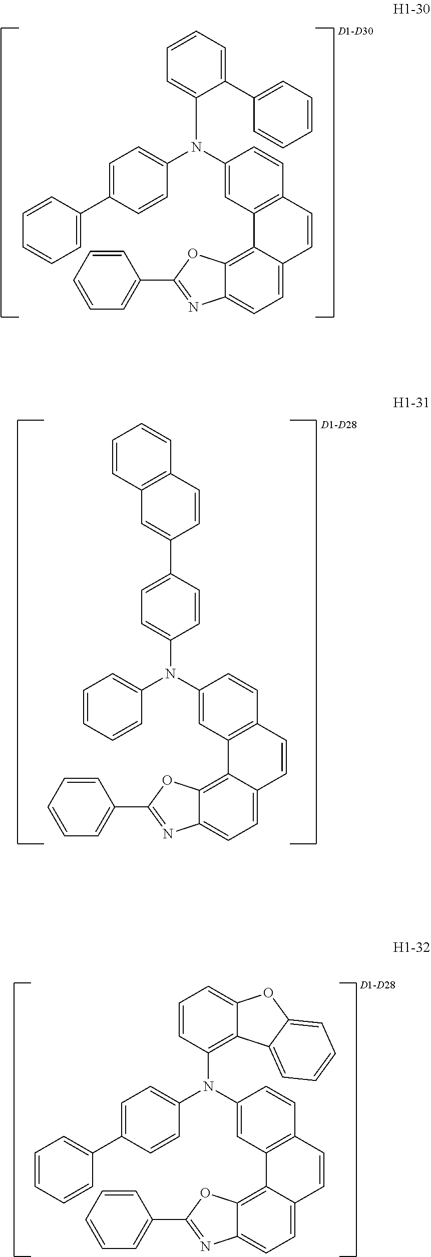

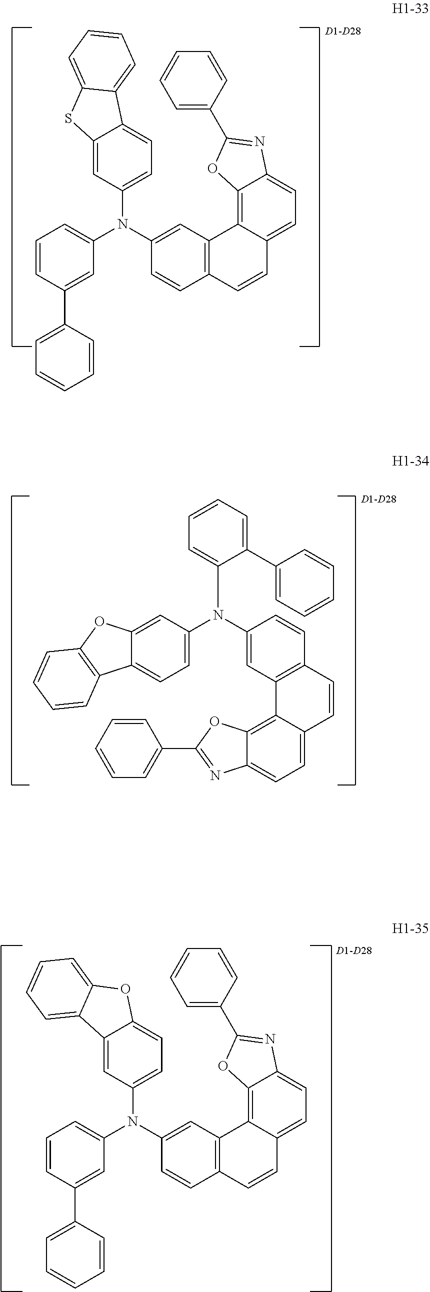

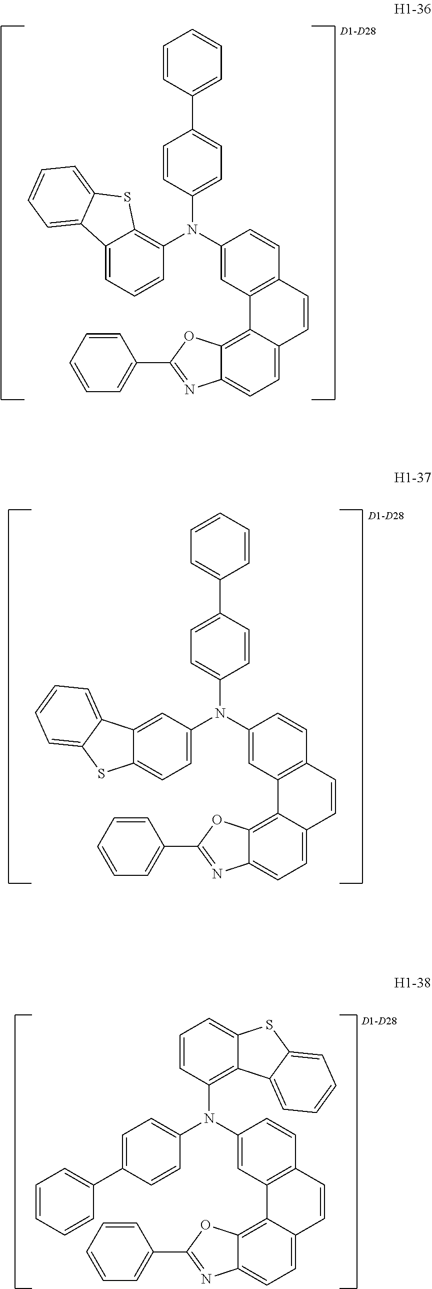

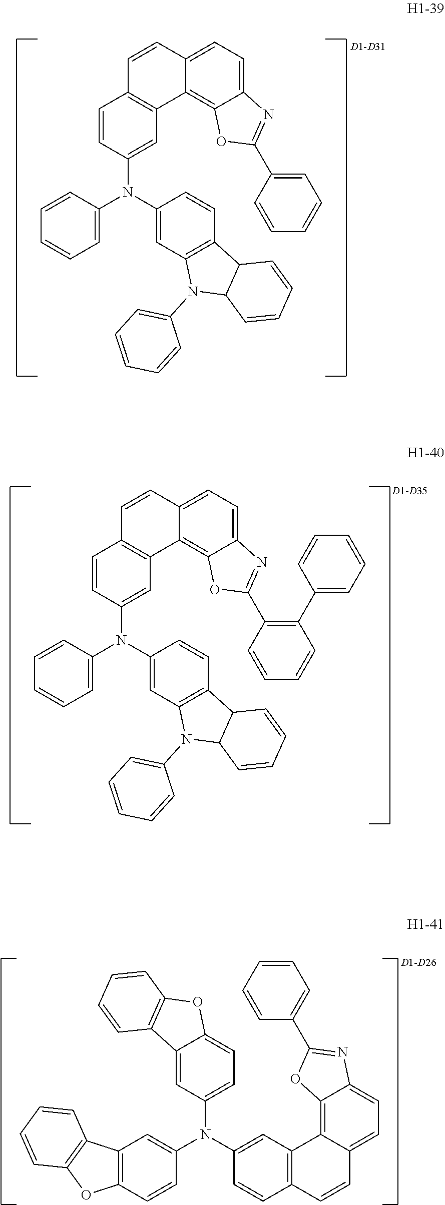

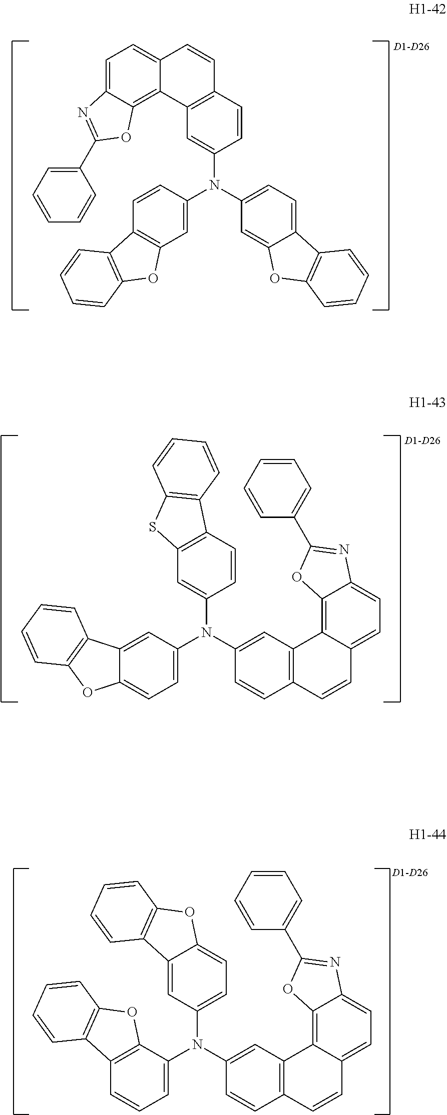

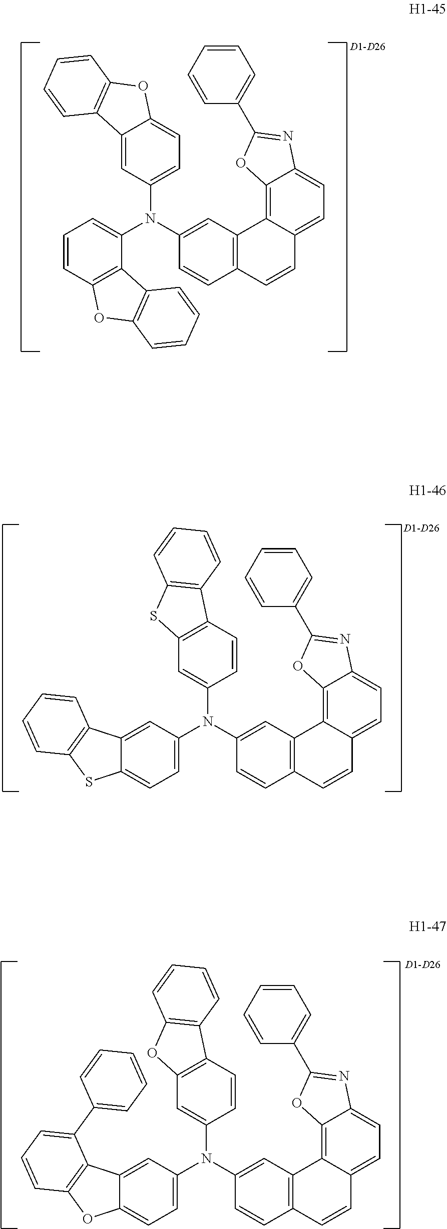

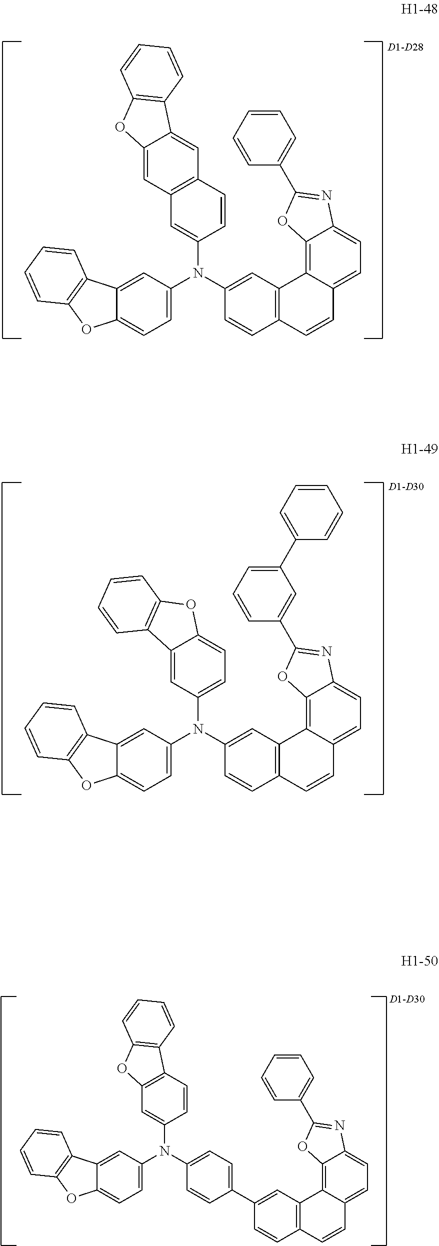

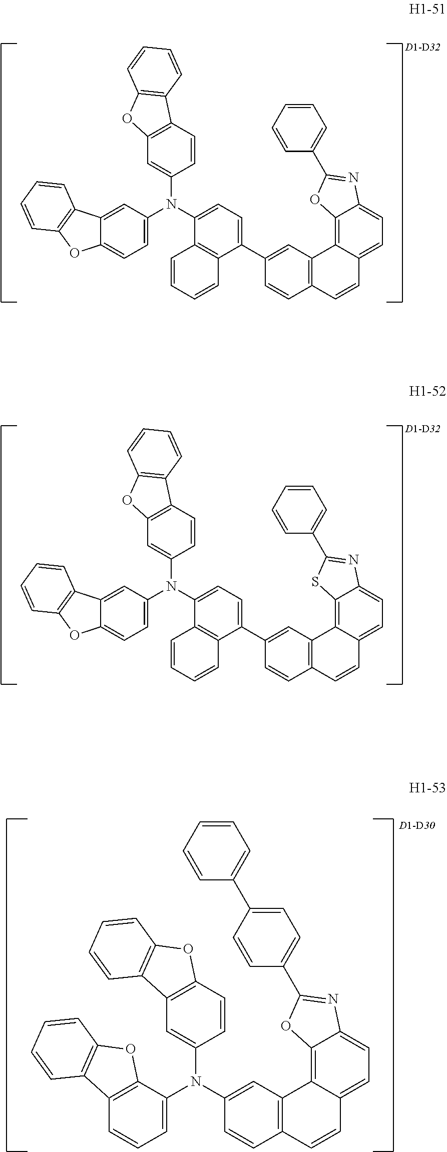

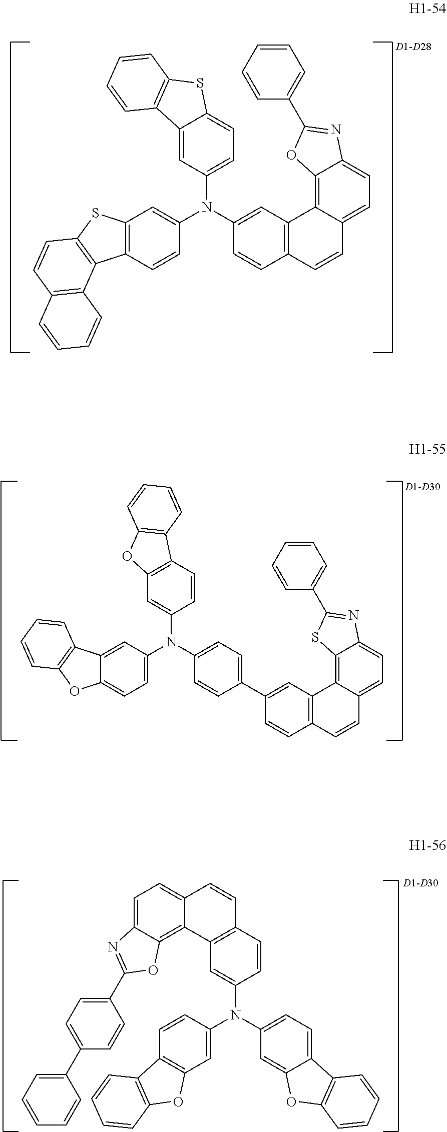

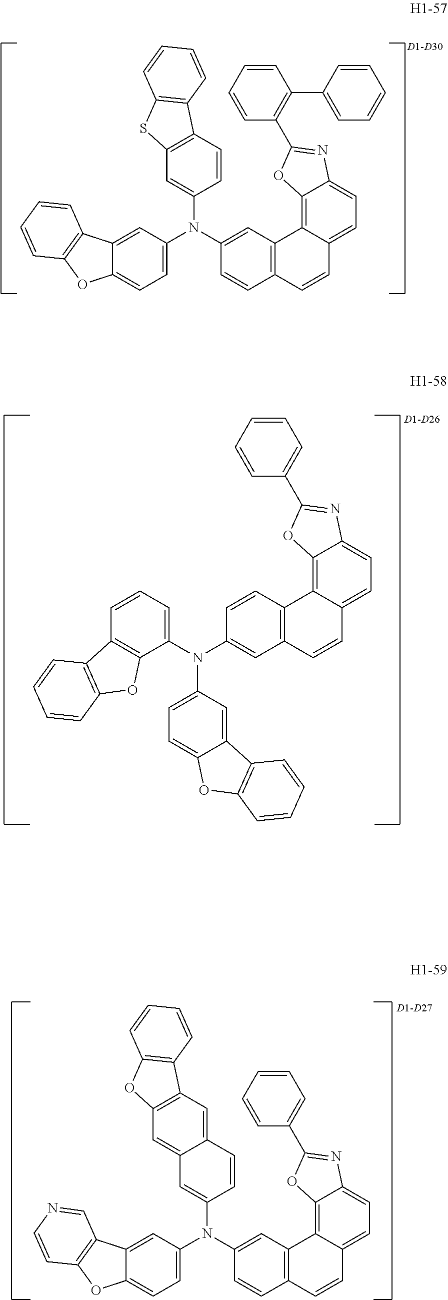

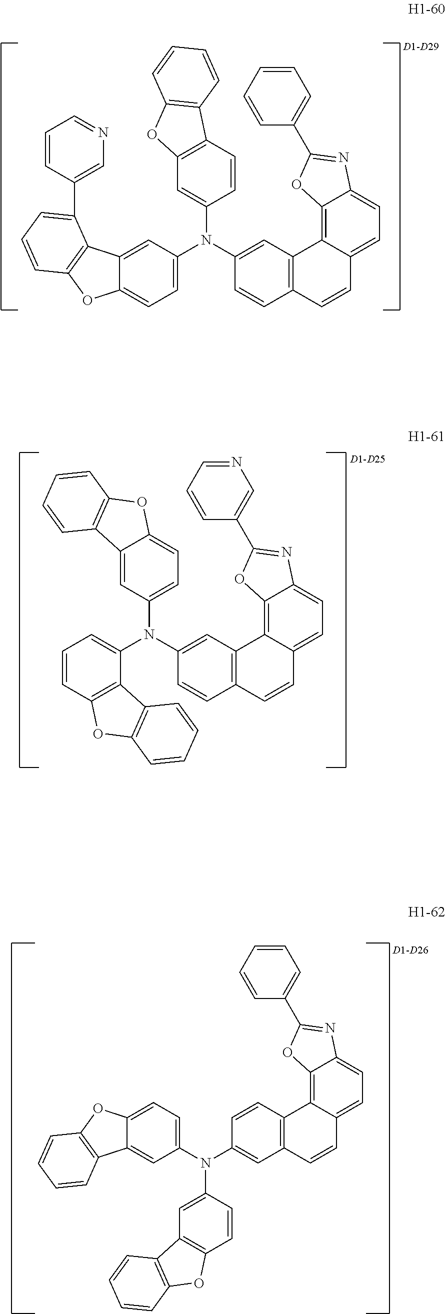

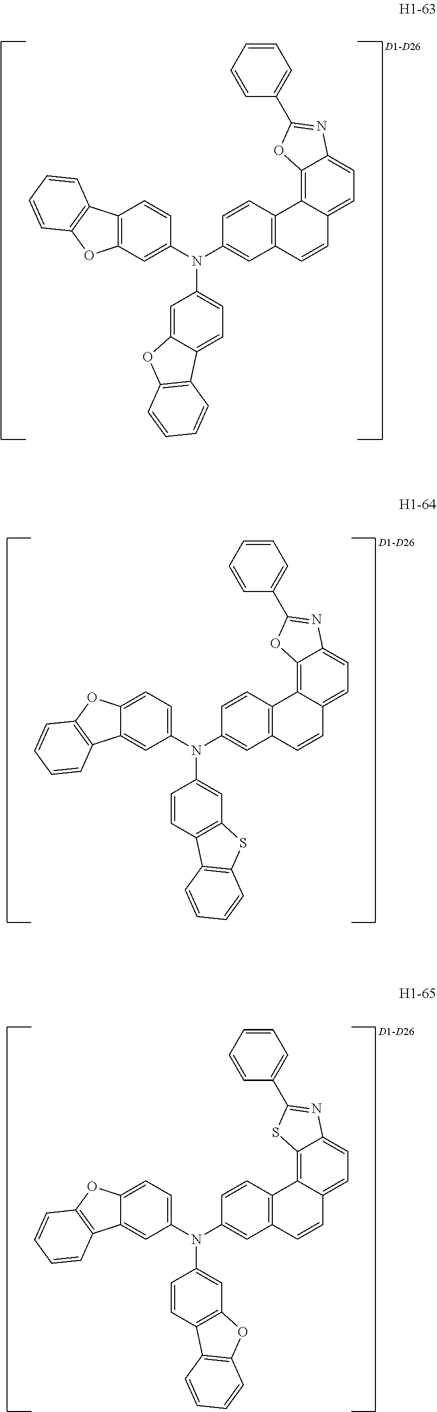

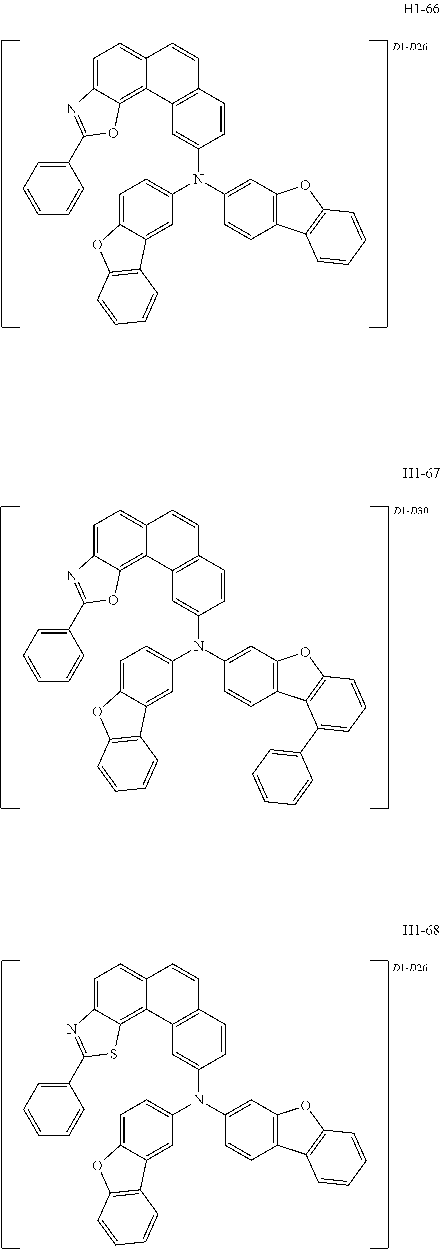

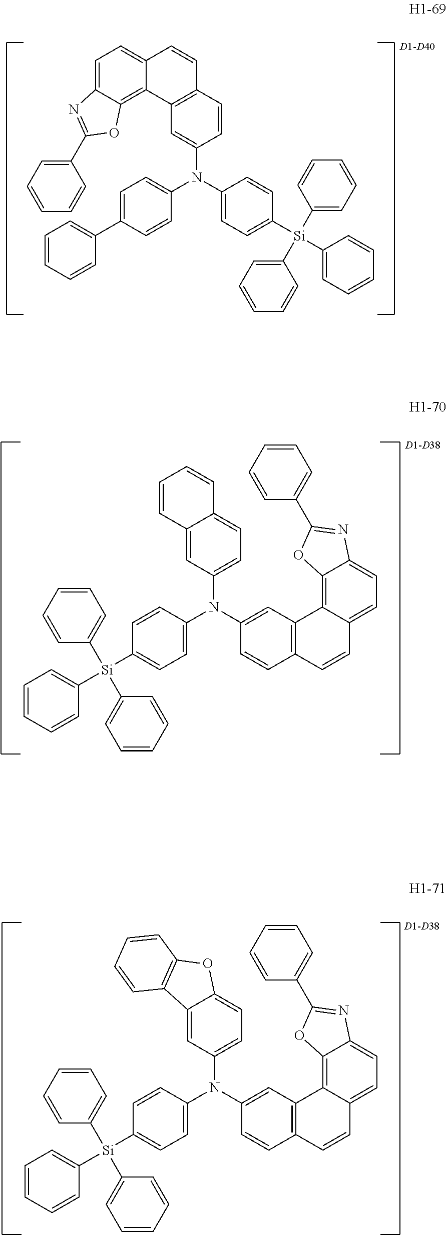

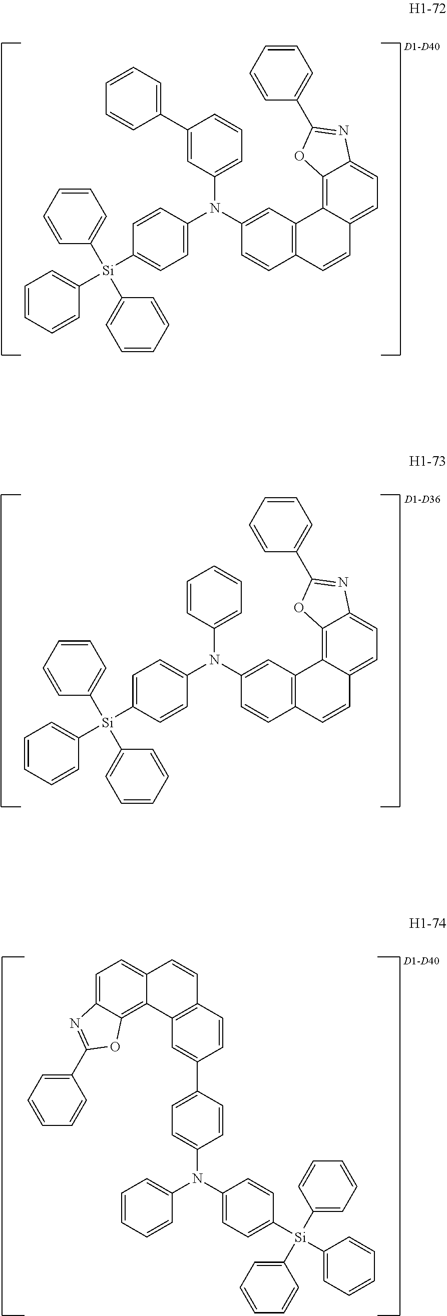

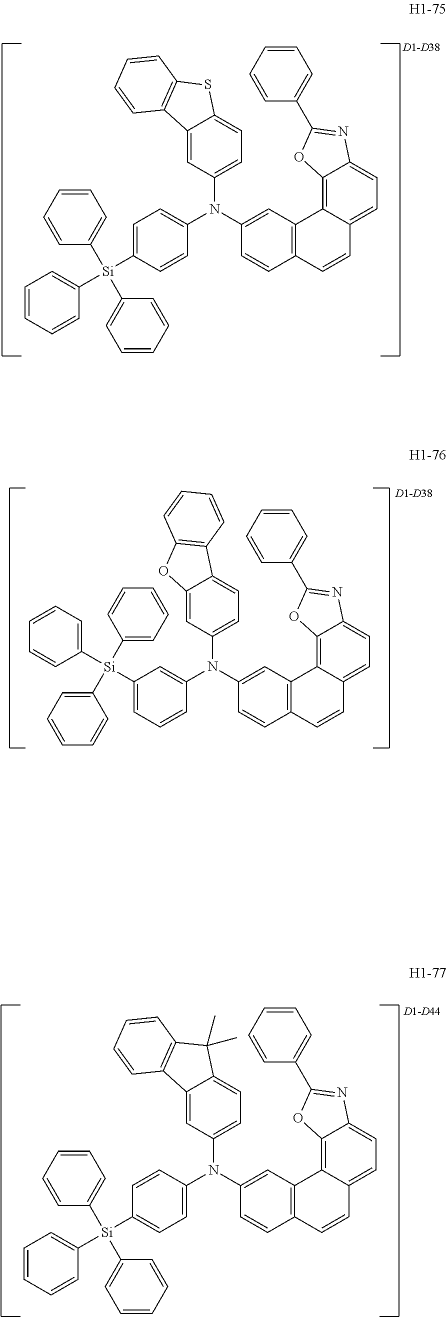

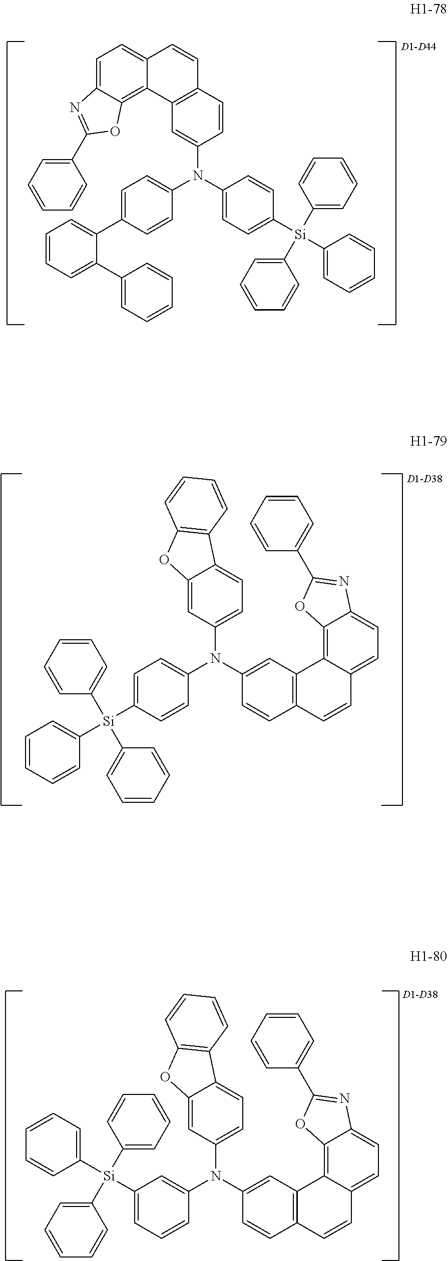

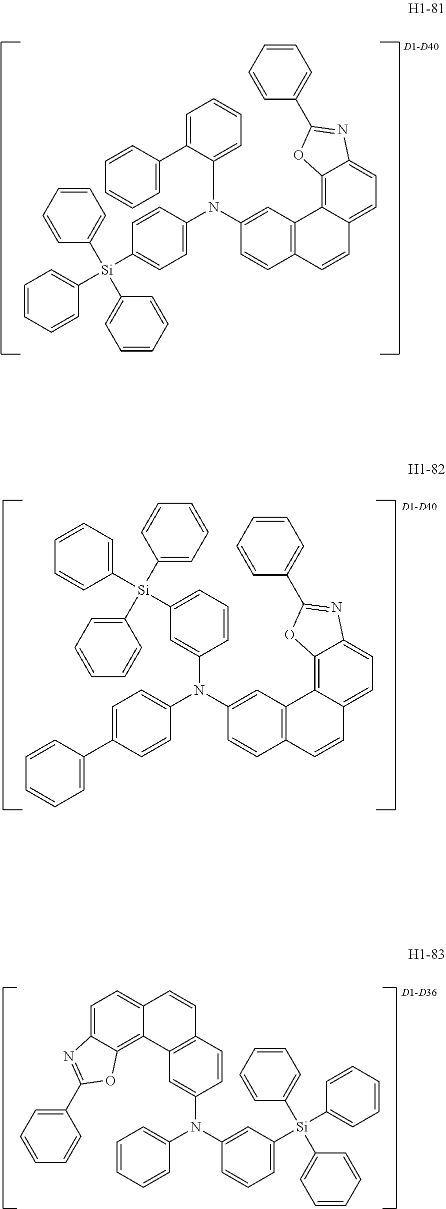

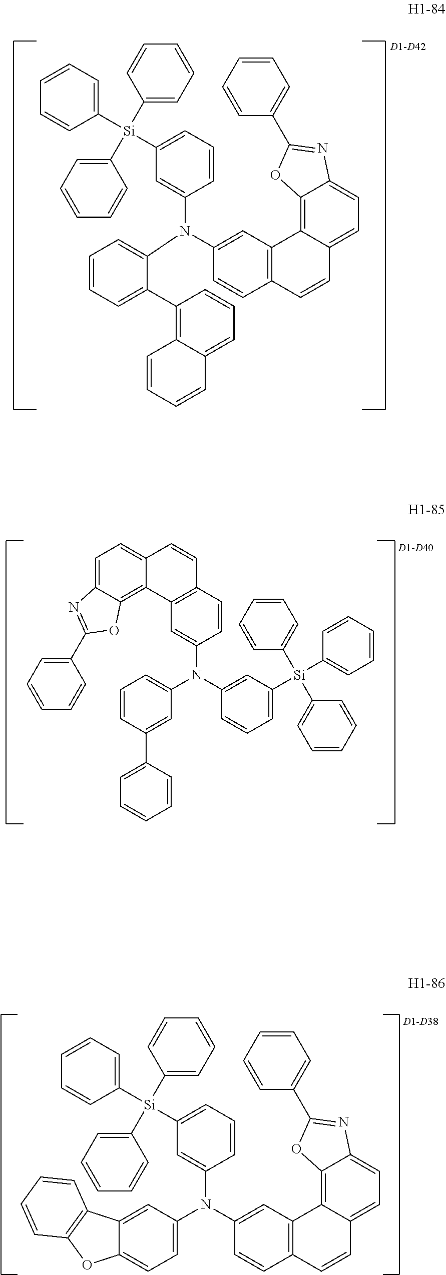

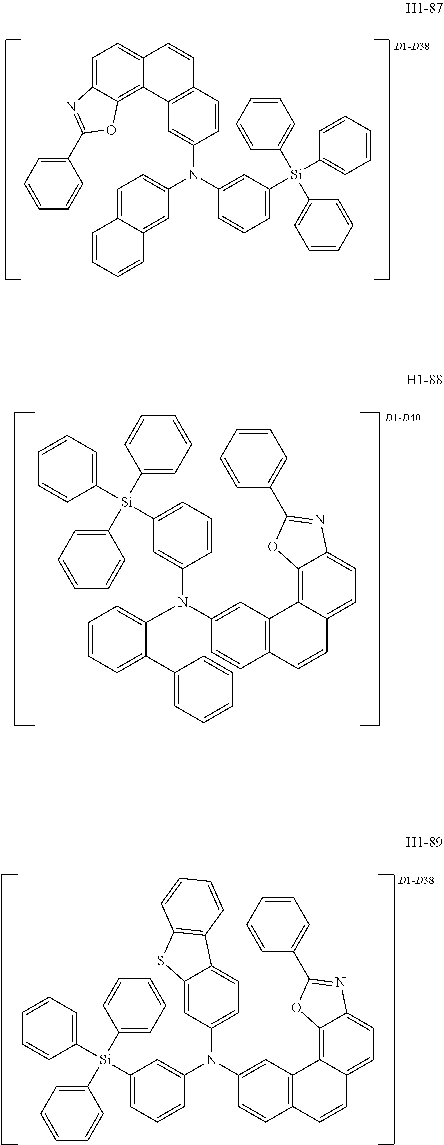

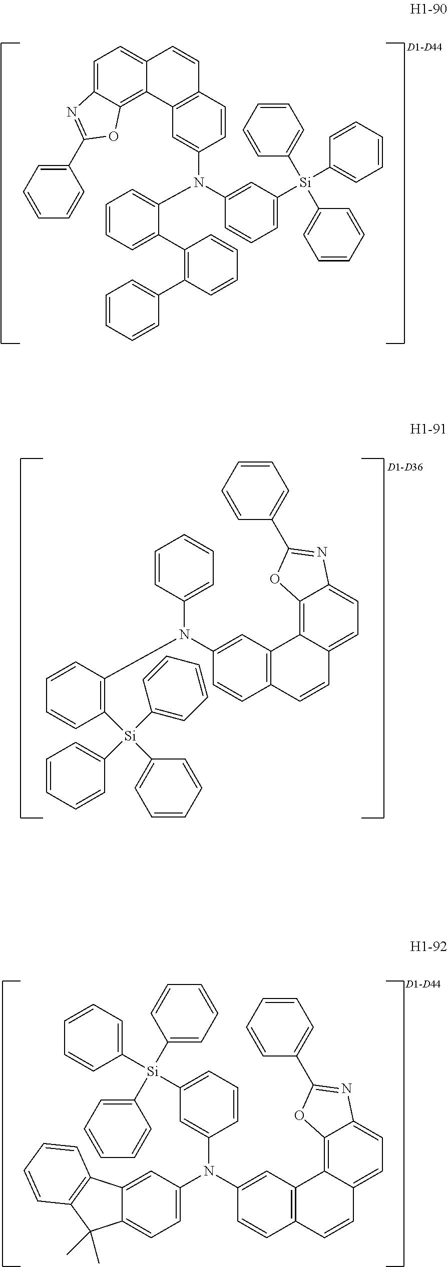

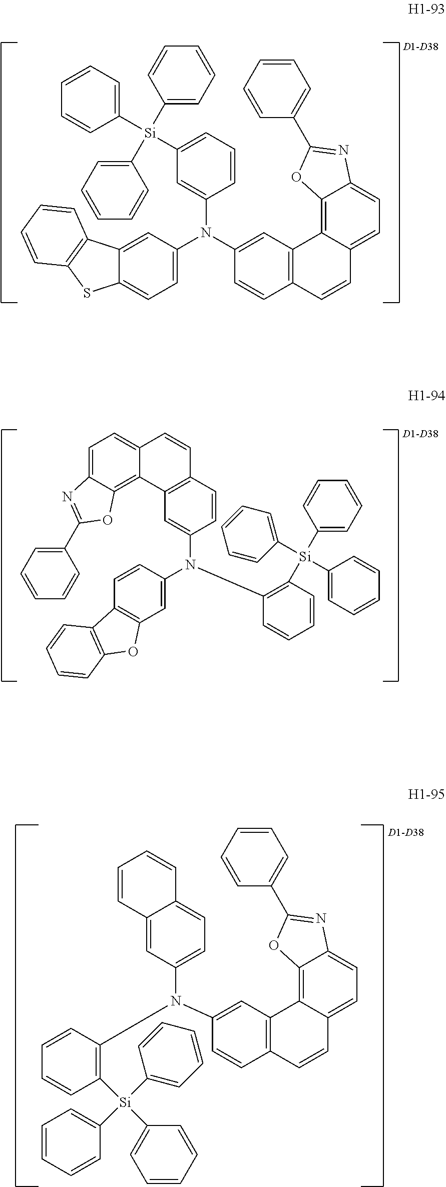

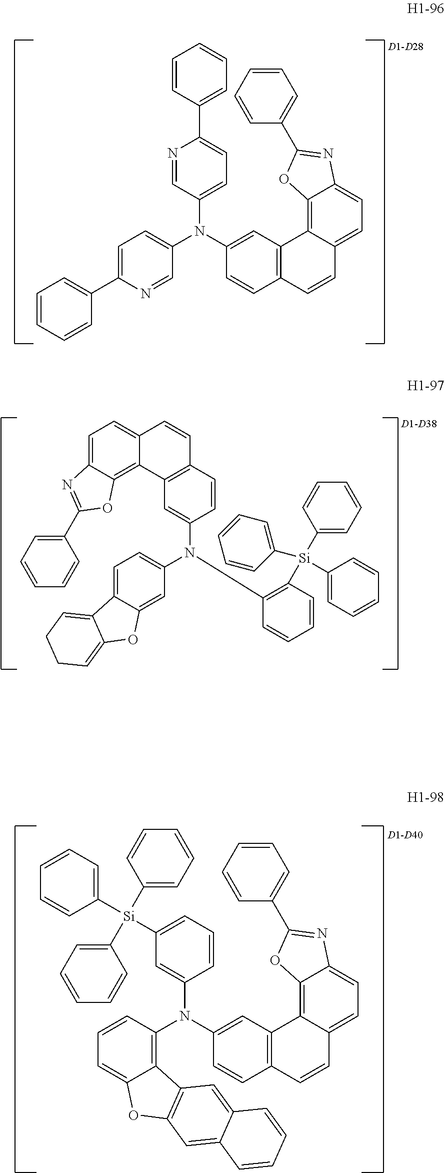

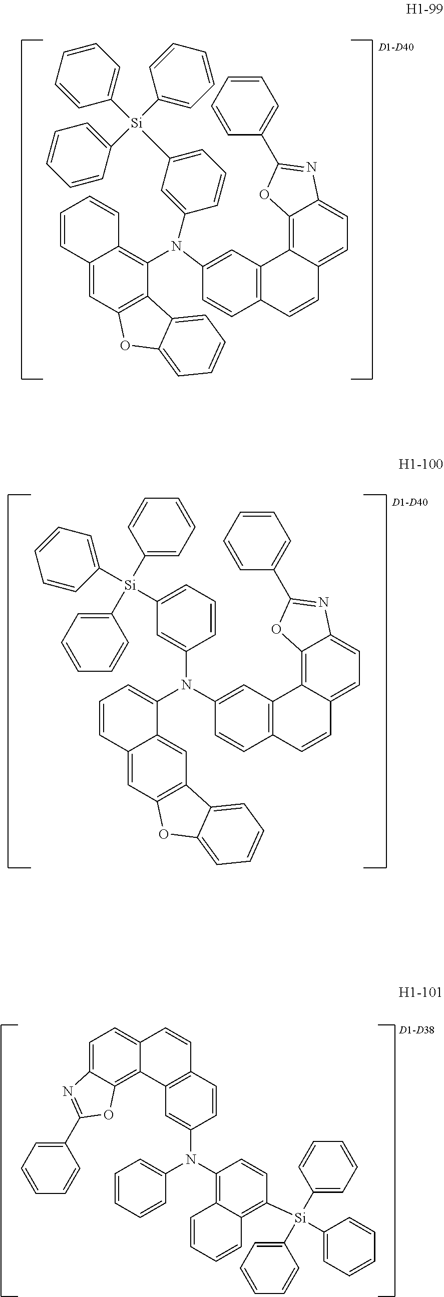

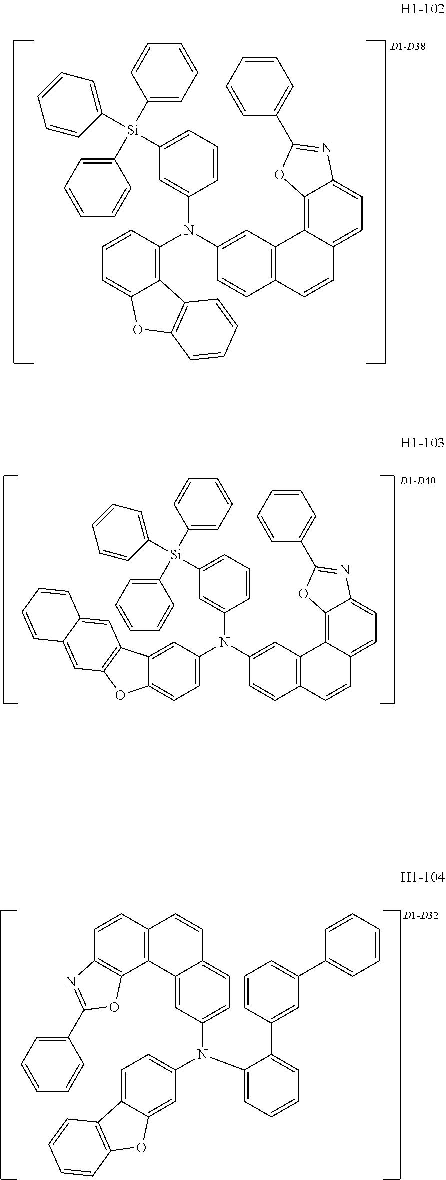

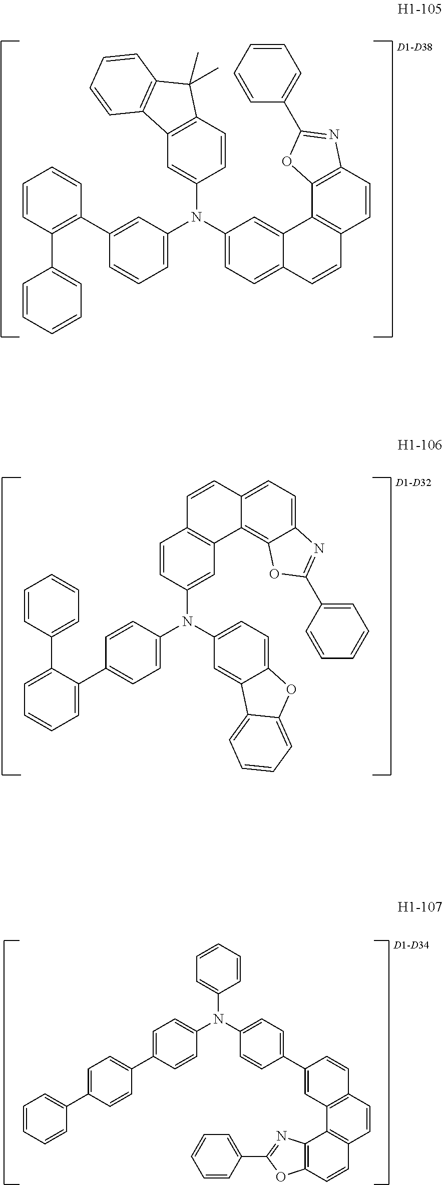

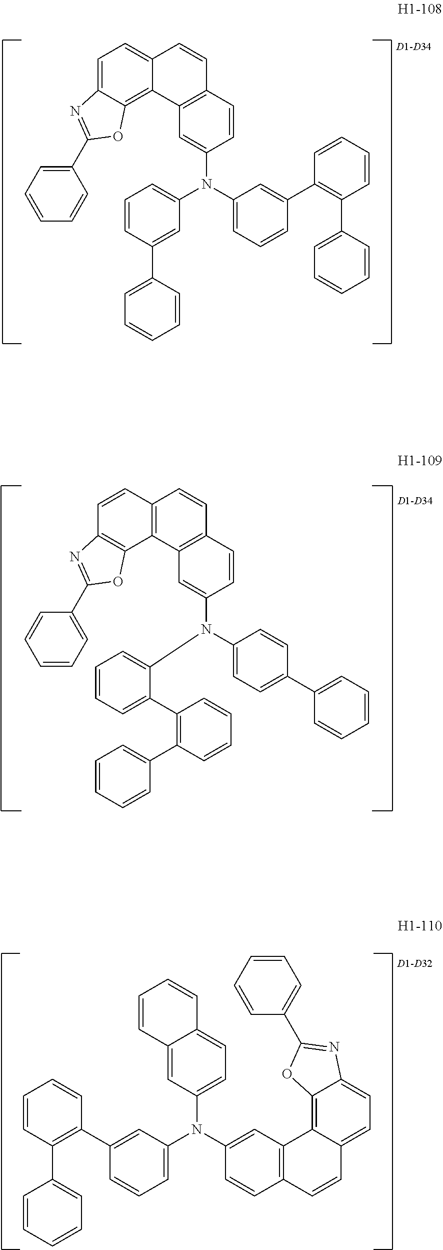

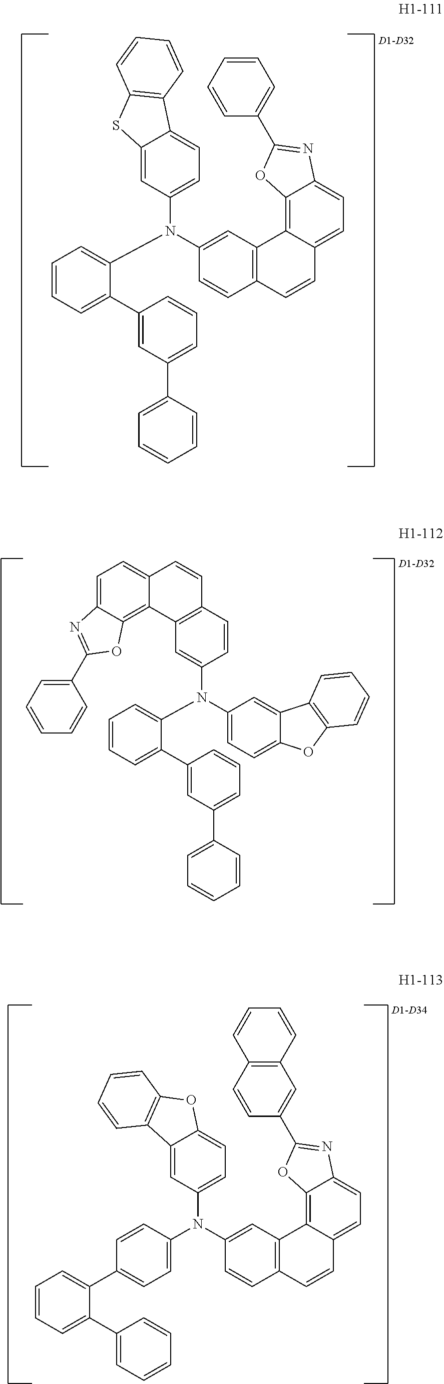

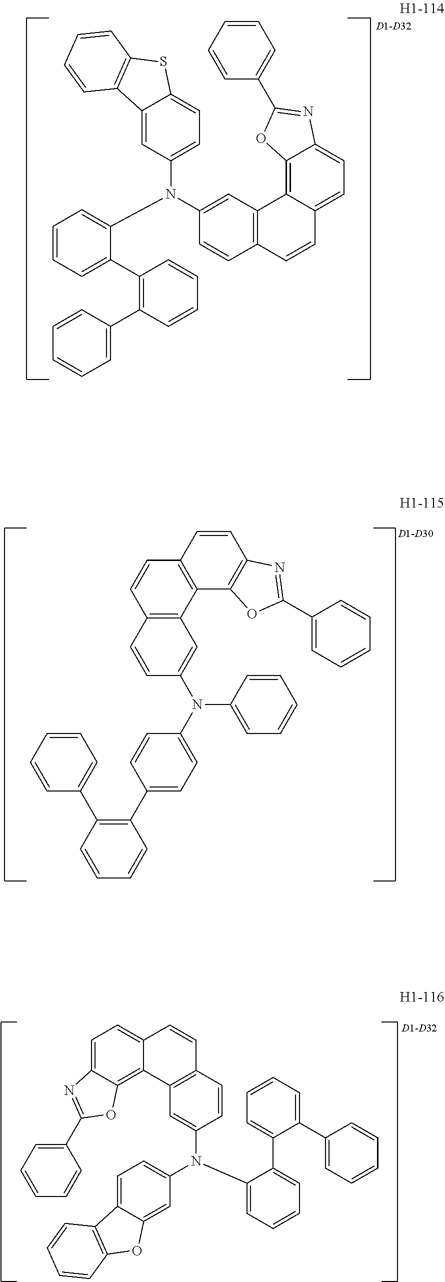

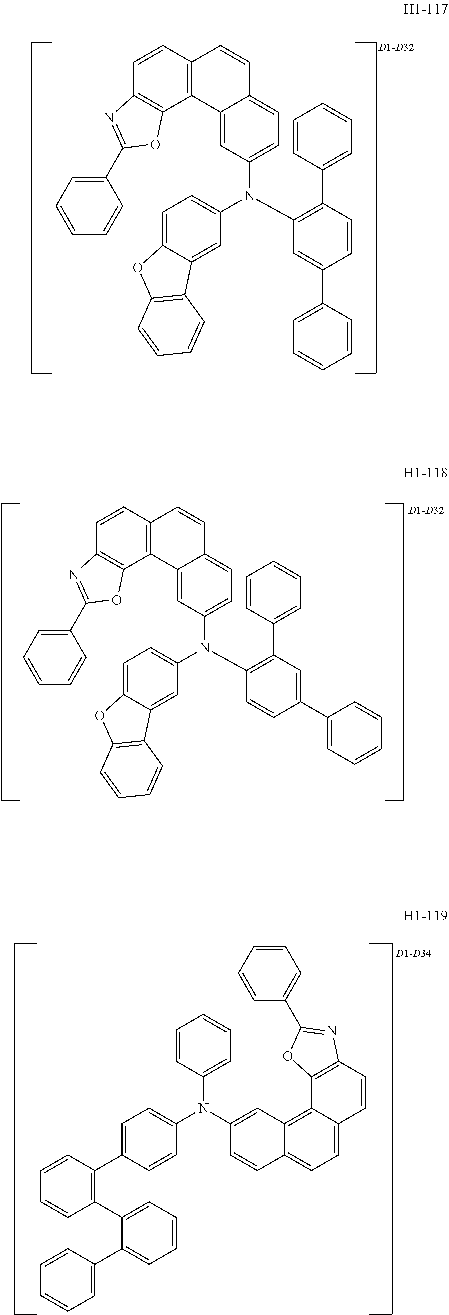

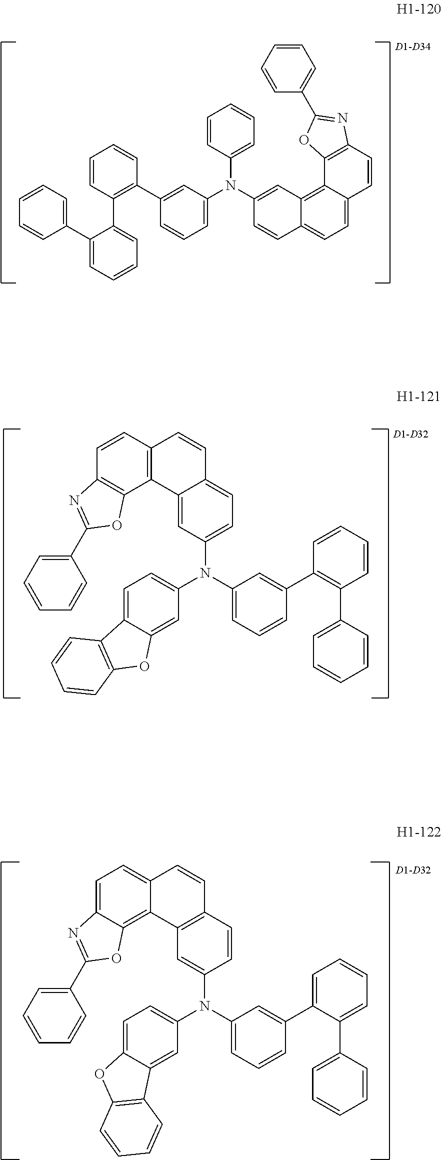

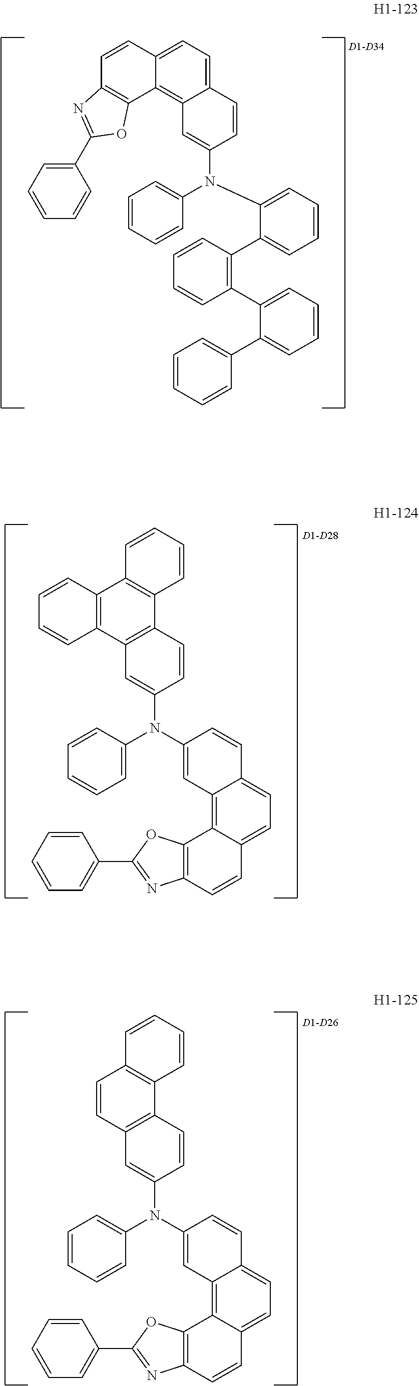

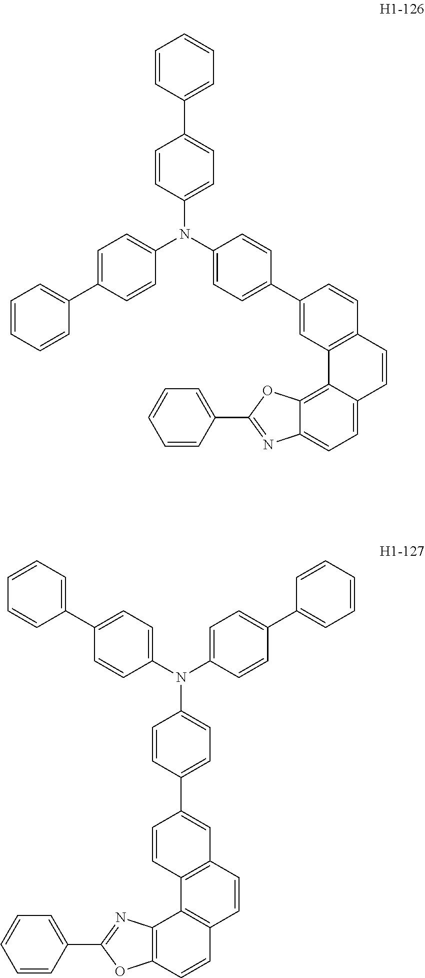 [SMILES: c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1]

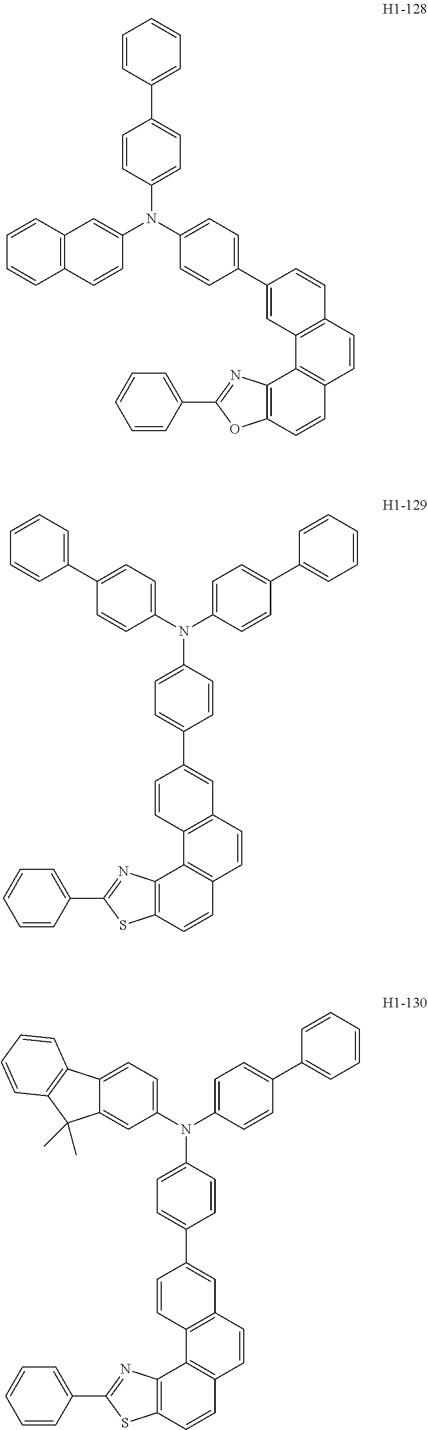 [SMILES: c1ccc(-c2ccc(N(c3ccc(-c4ccc5ccc6ccc7oc(-c8ccccc8)nc7c6c5c4)cc3)c3ccc4ccccc4c3)cc2)cc1 ; c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21]

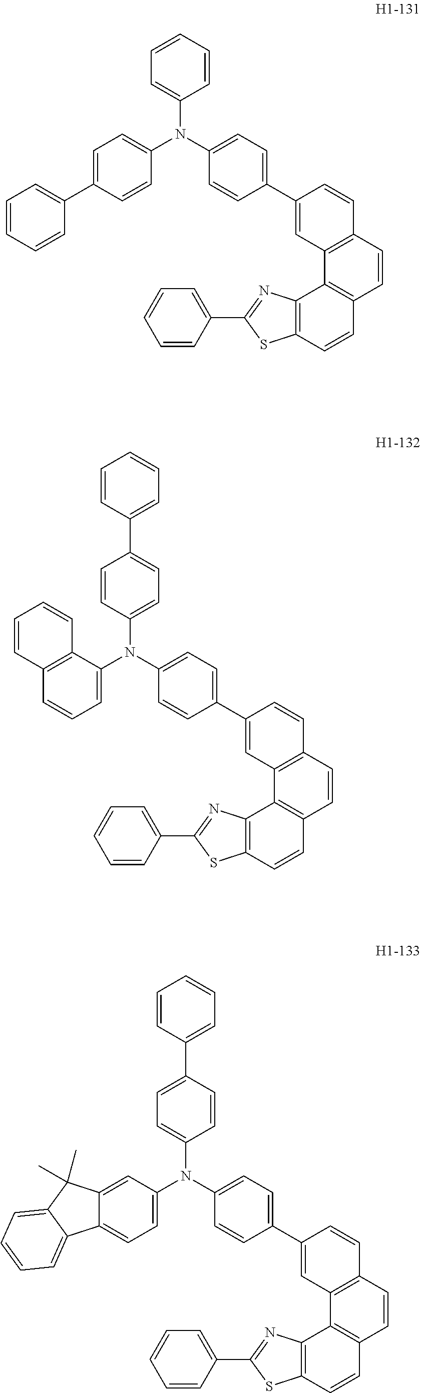 [SMILES: c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1 ; CC1(C)c2ccccc2-c2ccc(N(c3ccc(-c4ccccc4)cc3)c3ccc(-c4ccc5ccc6ccc7sc(-c8ccccc8)nc7c6c5c4)cc3)cc21]

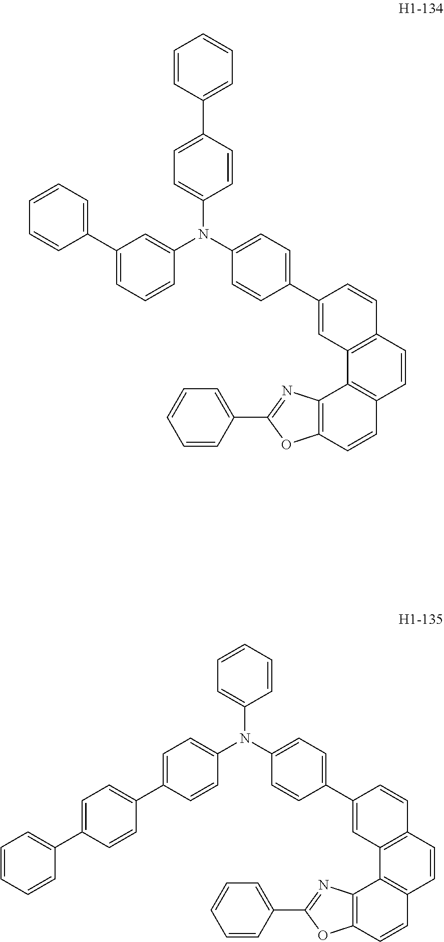 [SMILES: c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1]

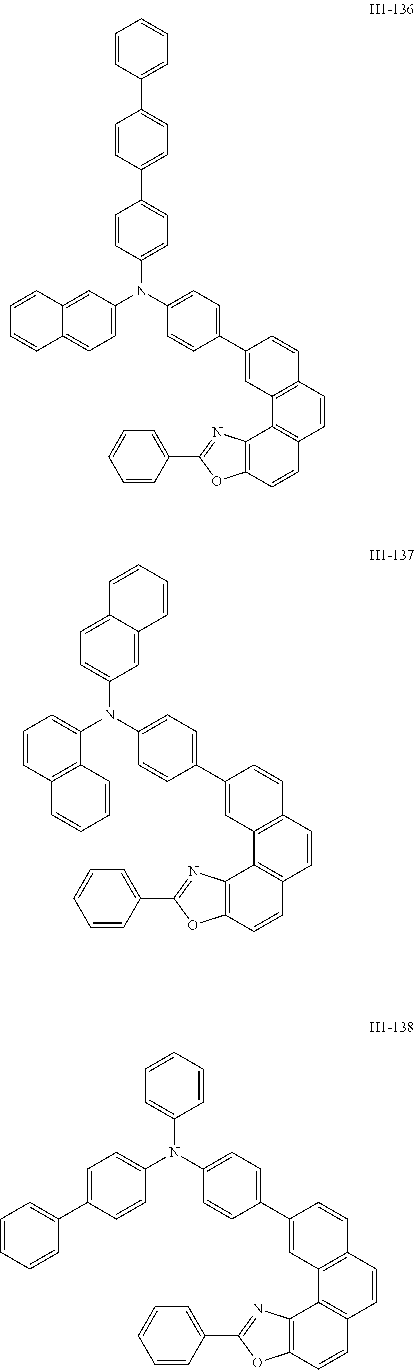 [SMILES: c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(N(c3ccccc3)c3ccc(-c4ccc5ccc6ccc7oc(-c8ccccc8)nc7c6c5c4)cc3)cc2)cc1]

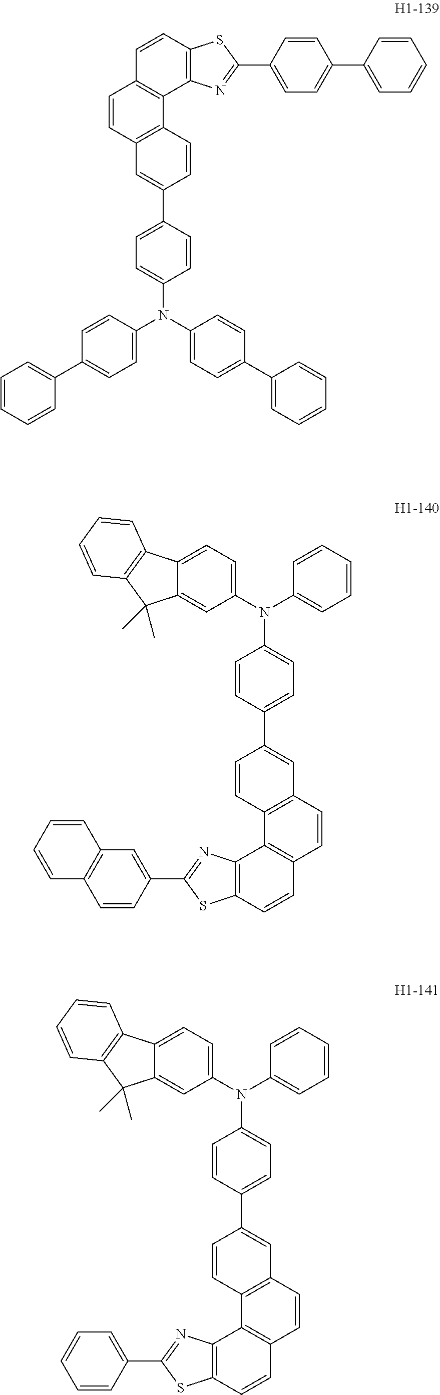 [SMILES: c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccccc3)c3ccc(-c4ccc5c(ccc6ccc7sc(-c8ccc9ccccc9c8)nc7c65)c4)cc3)cc21 ; CC1(C)c2ccccc2-c2ccc(N(c3ccccc3)c3ccc(-c4ccc5c(ccc6ccc7sc(-c8ccccc8)nc7c65)c4)cc3)cc21]

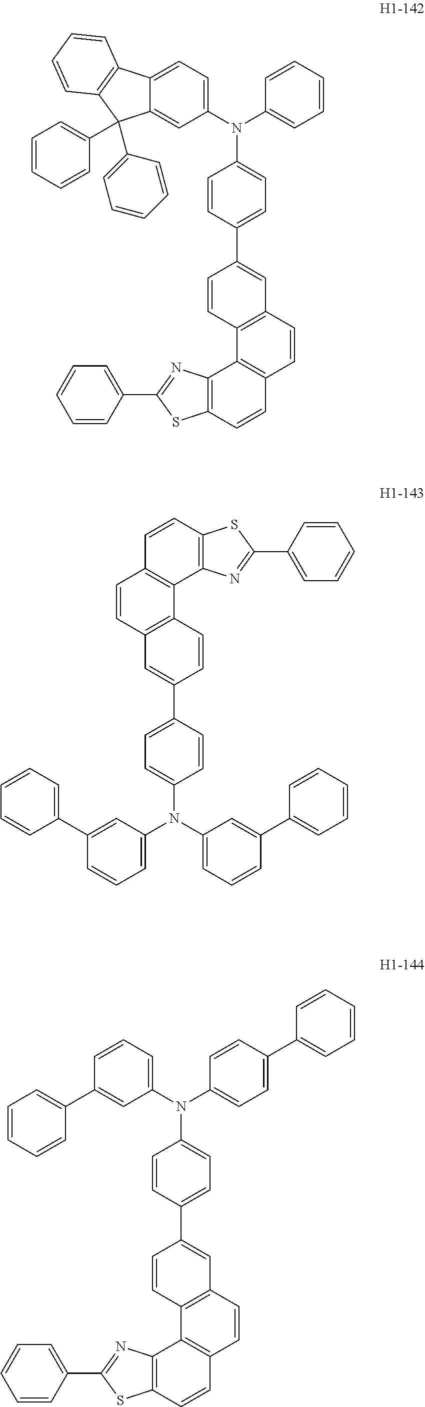 [SMILES: c1ccc(-c2nc3c(ccc4ccc5cc(-c6ccc(N(c7ccccc7)c7ccc8c(c7)C(c7ccccc7)(c7ccccc7)c7ccccc7-8)cc6)ccc5c43)s2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1]

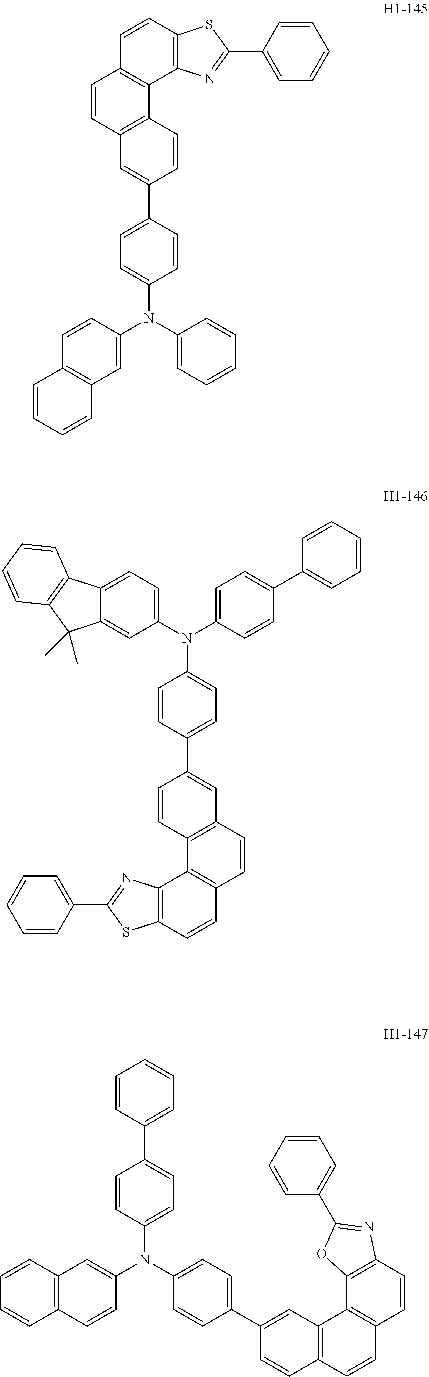 [SMILES: c1ccc(-c2nc3c(ccc4ccc5cc(-c6ccc(N(c7ccccc7)c7ccc8ccccc8c7)cc6)ccc5c43)s2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; c1ccc(-c2ccc(N(c3ccc(-c4ccc5ccc6ccc7nc(-c8ccccc8)oc7c6c5c4)cc3)c3ccc4ccccc4c3)cc2)cc1]

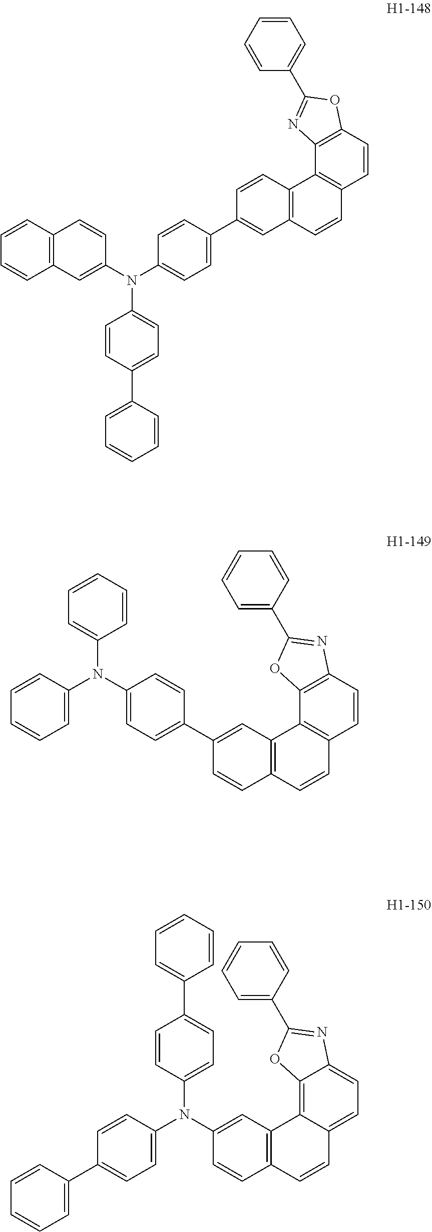 [SMILES: c1ccc(-c2ccc(N(c3ccc(-c4ccc5c(ccc6ccc7oc(-c8ccccc8)nc7c65)c4)cc3)c3ccc4ccccc4c3)cc2)cc1 ; c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1]

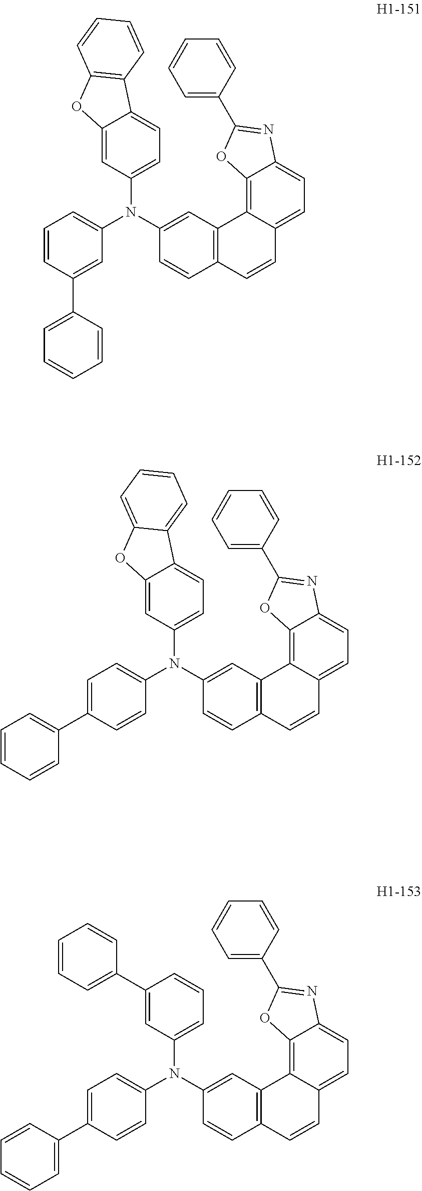 [SMILES: c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3ccc4c(c3)oc3ccccc34)c3ccc4ccc5ccc6nc(-c7ccccc7)oc6c5c4c3)cc2)cc1 ; c1ccc(-c2ccc(N(c3cccc(-c4ccccc4)c3)c3ccc4ccc5ccc6nc(-c7ccccc7)oc6c5c4c3)cc2)cc1]

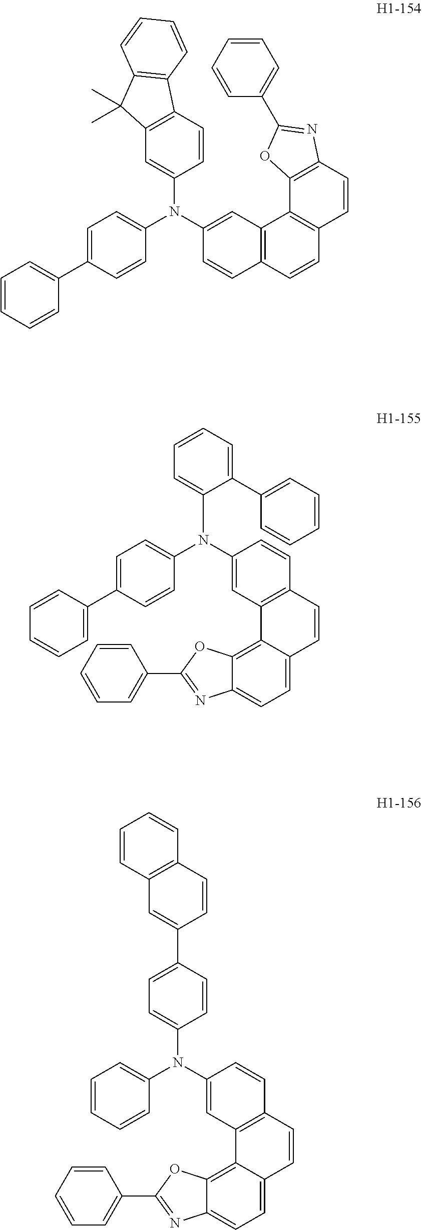 [SMILES: CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc4ccc5ccc6nc(-c7ccccc7)oc6c5c4c3)c3ccccc3-c3ccccc3)cc2)cc1 ; c1ccc(-c2nc3ccc4ccc5ccc(N(c6ccccc6)c6ccc(-c7ccc8ccccc8c7)cc6)cc5c4c3o2)cc1]

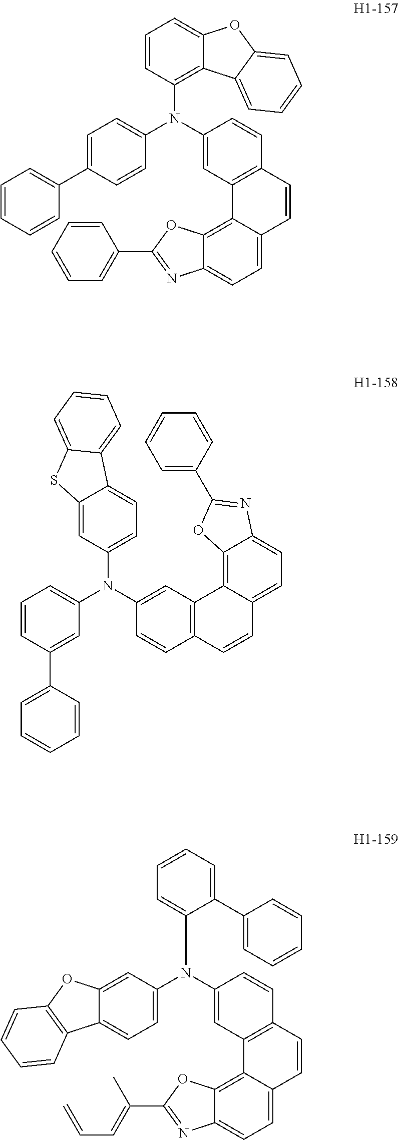 [SMILES: c1ccc(-c2ccc(N(c3ccc4ccc5ccc6nc(-c7ccccc7)oc6c5c4c3)c3cccc4oc5ccccc5c34)cc2)cc1 ; c1ccc(-c2cccc(N(c3ccc4c(c3)sc3ccccc34)c3ccc4ccc5ccc6nc(-c7ccccc7)oc6c5c4c3)c2)cc1 ; C=C/C=C(\C)c1nc2ccc3ccc4ccc(N(c5ccc6c(c5)oc5ccccc56)c5ccccc5-c5ccccc5)cc4c3c2o1]

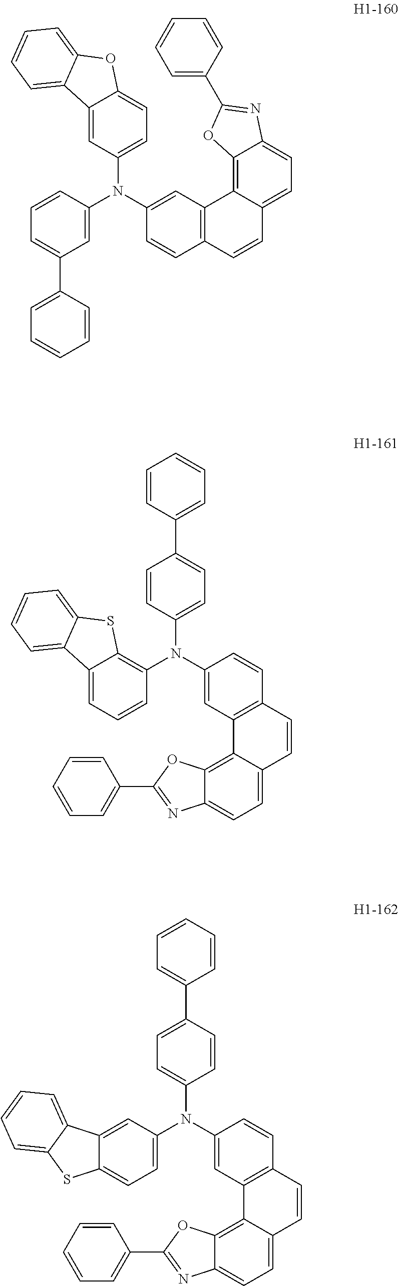 [SMILES: c1ccc(-c2cccc(N(c3ccc4oc5ccccc5c4c3)c3ccc4ccc5ccc6nc(-c7ccccc7)oc6c5c4c3)c2)cc1 ; c1ccc(-c2ccc(N(c3ccc4ccc5ccc6nc(-c7ccccc7)oc6c5c4c3)c3cccc4c3sc3ccccc34)cc2)cc1 ; c1ccc(-c2ccc(N(c3ccc4sc5ccccc5c4c3)c3ccc4ccc5ccc6nc(-c7ccccc7)oc6c5c4c3)cc2)cc1]

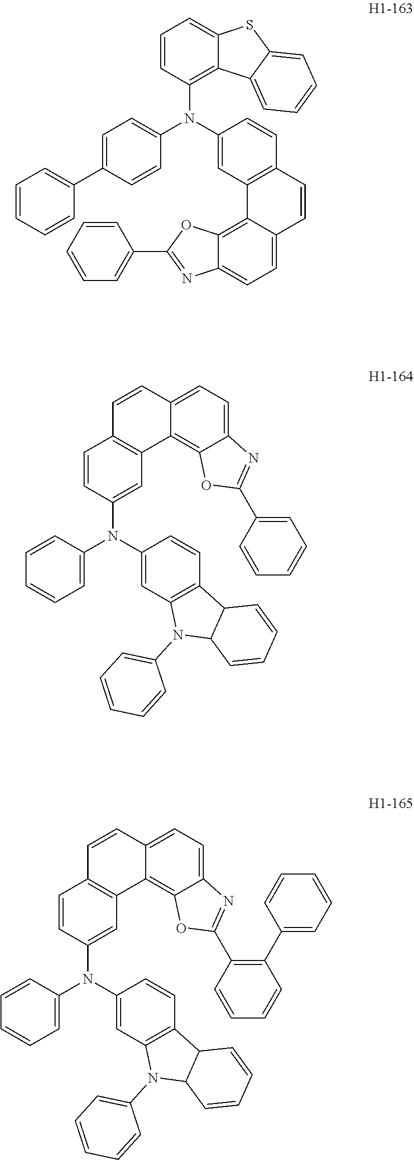 [SMILES: c1ccc(-c2ccc(N(c3ccc4ccc5ccc6nc(-c7ccccc7)oc6c5c4c3)c3cccc4sc5ccccc5c34)cc2)cc1 ; C1=CC2c3ccc(N(c4ccccc4)c4ccc5ccc6ccc7nc(-c8ccccc8)oc7c6c5c4)cc3N(c3ccccc3)C2C=C1 ; C1=CC2c3ccc(N(c4ccccc4)c4ccc5ccc6ccc7nc(-c8ccccc8-c8ccccc8)oc7c6c5c4)cc3N(c3ccccc3)C2C=C1]

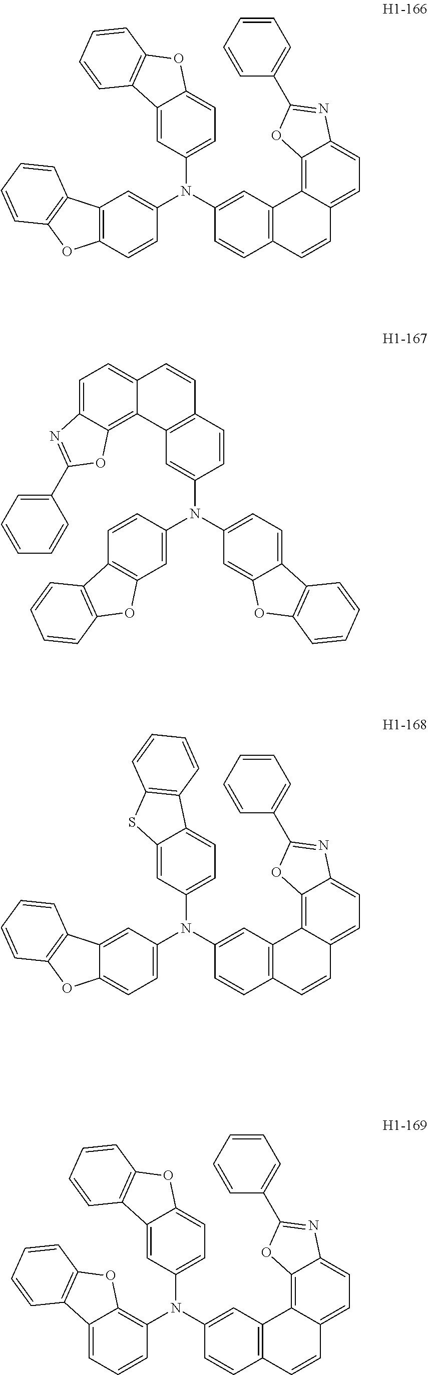 [SMILES: c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7c(c6)oc6ccccc67)cc5c4c3o2)cc1 ; c1ccc(-c2nc3ccc4ccc5ccc(N(c6ccc7c(c6)sc6ccccc67)c6ccc7oc8ccccc8c7c6)cc5c4c3o2)cc1 ; c1ccc(-c2nc3ccc4ccc5ccc(N(c6ccc7oc8ccccc8c7c6)c6cccc7c6oc6ccccc67)cc5c4c3o2)cc1]

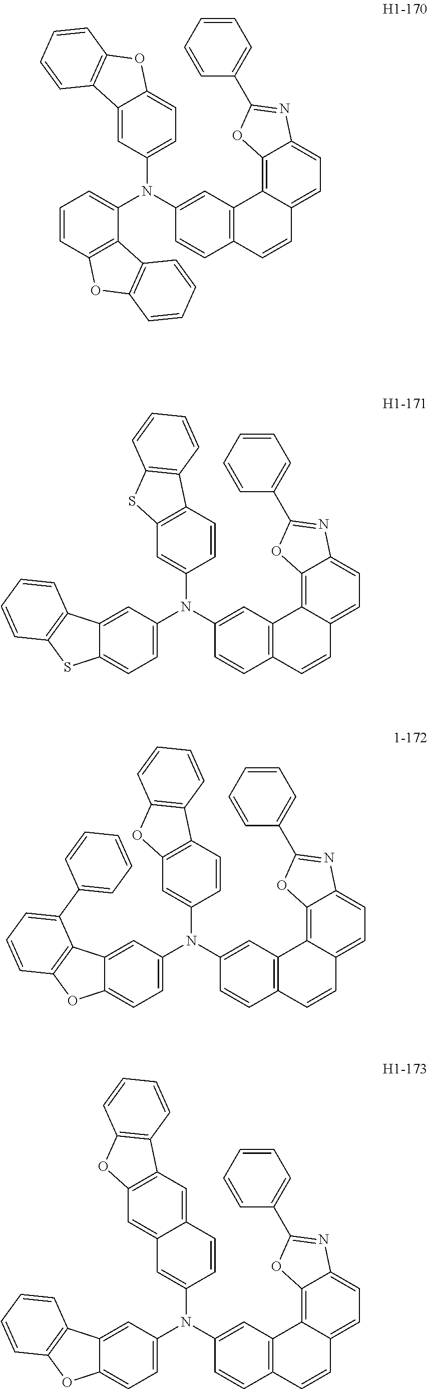 [SMILES: c1ccc(-c2nc3ccc4ccc5ccc(N(c6ccc7oc8ccccc8c7c6)c6cccc7oc8ccccc8c67)cc5c4c3o2)cc1 ; c1ccc(-c2nc3ccc4ccc5ccc(N(c6ccc7c(c6)sc6ccccc67)c6ccc7sc8ccccc8c7c6)cc5c4c3o2)cc1 ; c1ccc(-c2nc3ccc4ccc5ccc(N(c6ccc7c(c6)oc6ccccc67)c6ccc7oc8cccc(-c9ccccc9)c8c7c6)cc5c4c3o2)cc1 ; c1ccc(-c2nc3ccc4ccc5ccc(N(c6ccc7cc8c(cc7c6)oc6ccccc68)c6ccc7oc8ccccc8c7c6)cc5c4c3o2)cc1]

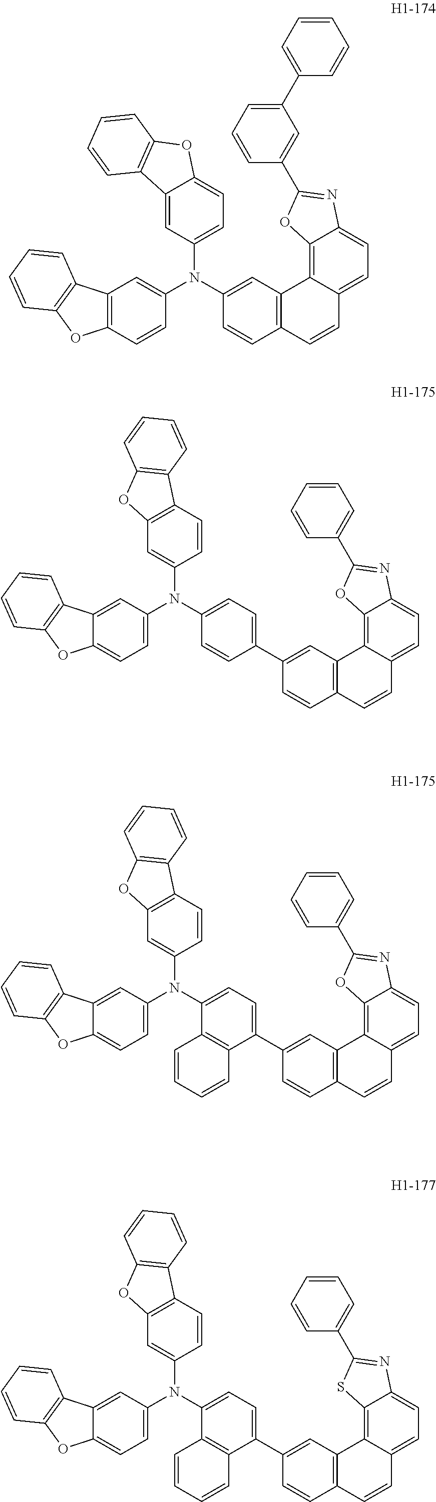 [SMILES: c1ccc(-c2cccc(-c3nc4ccc5ccc6ccc(N(c7ccc8oc9ccccc9c8c7)c7ccc8oc9ccccc9c8c7)cc6c5c4o3)c2)cc1 ; c1ccc(-c2nc3ccc4ccc5ccc(-c6ccc(N(c7ccc8c(c7)oc7ccccc78)c7ccc8oc9ccccc9c8c7)cc6)cc5c4c3o2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3o2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3s2)cc1]

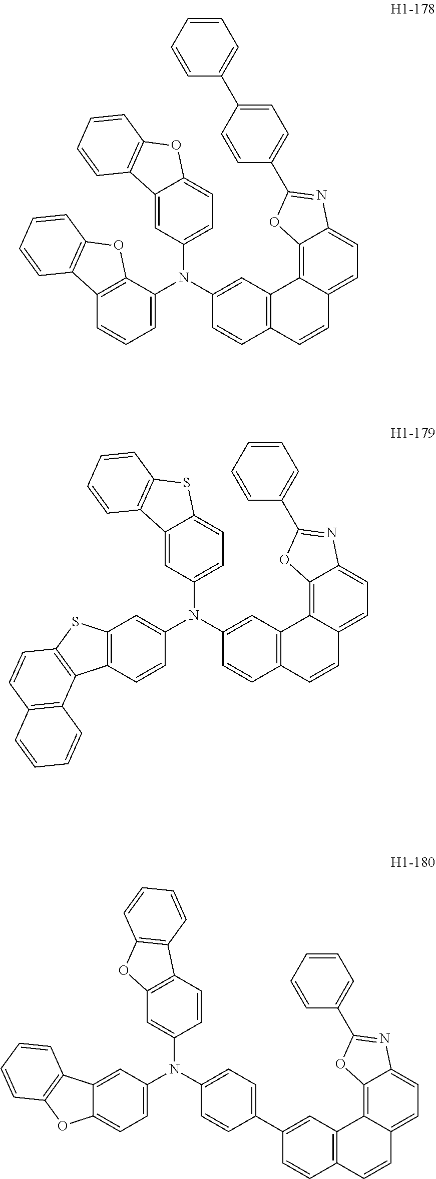 [SMILES: c1ccc(-c2ccc(-c3nc4ccc5ccc6ccc(N(c7ccc8oc9ccccc9c8c7)c7cccc8c7oc7ccccc78)cc6c5c4o3)cc2)cc1 ; c1ccc(-c2nc3ccc4ccc5ccc(N(c6ccc7c(c6)sc6ccc8ccccc8c67)c6ccc7sc8ccccc8c7c6)cc5c4c3o2)cc1 ; c1ccc(-c2nc3ccc4ccc5ccc(-c6ccc(N(c7ccc8c(c7)oc7ccccc78)c7ccc8oc9ccccc9c8c7)cc6)cc5c4c3o2)cc1]

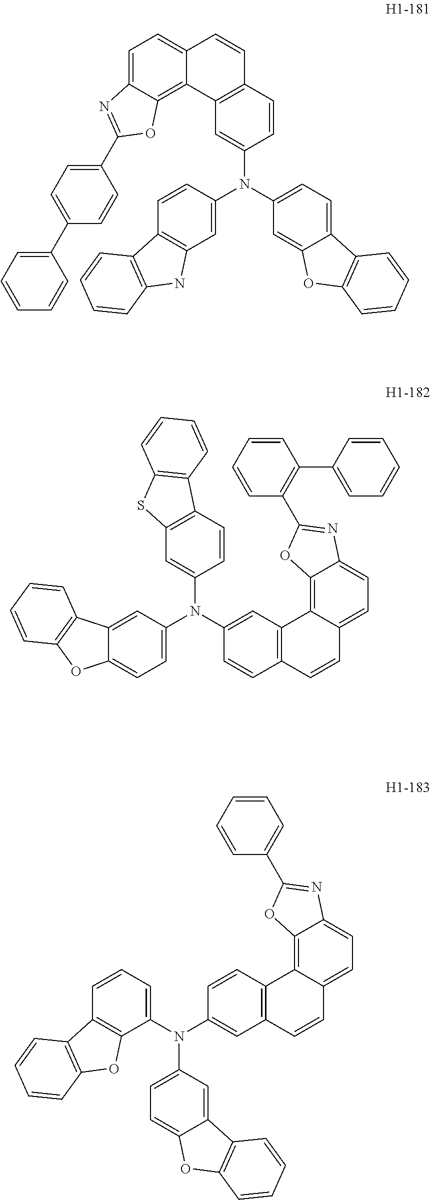 [SMILES: c1ccc(-c2ccc(-c3nc4ccc5ccc6ccc(N(c7ccc8c(c7)[nH]c7ccccc78)c7ccc8c(c7)oc7ccccc78)cc6c5c4o3)cc2)cc1 ; c1ccc(-c2ccccc2-c2nc3ccc4ccc5ccc(N(c6ccc7c(c6)sc6ccccc67)c6ccc7oc8ccccc8c7c6)cc5c4c3o2)cc1 ; c1ccc(-c2nc3ccc4ccc5cc(N(c6ccc7oc8ccccc8c7c6)c6cccc7c6oc6ccccc67)ccc5c4c3o2)cc1]

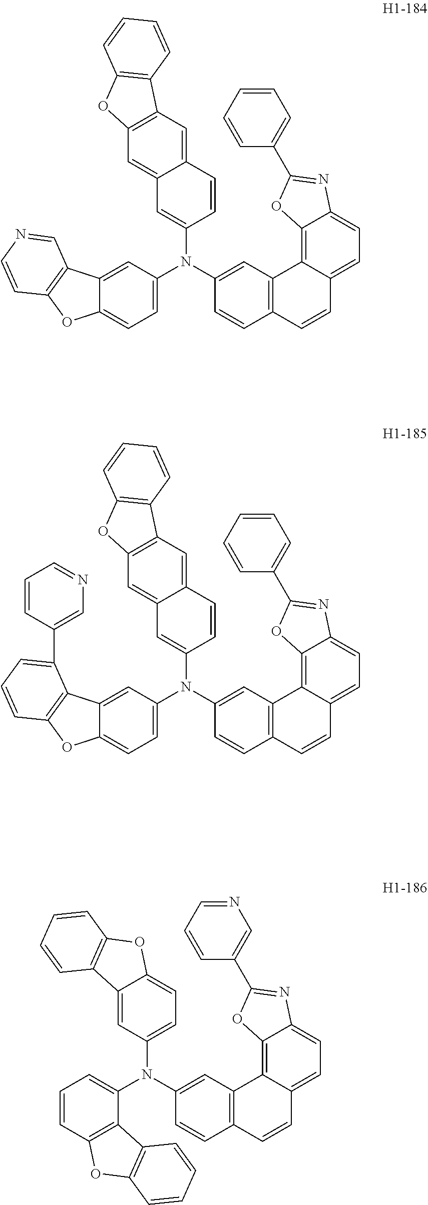 [SMILES: c1ccc(-c2nc3ccc4ccc5ccc(N(c6ccc7cc8c(cc7c6)oc6ccccc68)c6ccc7oc8ccncc8c7c6)cc5c4c3o2)cc1 ; c1ccc(-c2nc3ccc4ccc5ccc(N(c6ccc7cc8c(cc7c6)oc6ccccc68)c6ccc7oc8cccc(-c9cccnc9)c8c7c6)cc5c4c3o2)cc1 ; c1cncc(-c2nc3ccc4ccc5ccc(N(c6ccc7oc8ccccc8c7c6)c6cccc7oc8ccccc8c67)cc5c4c3o2)c1]

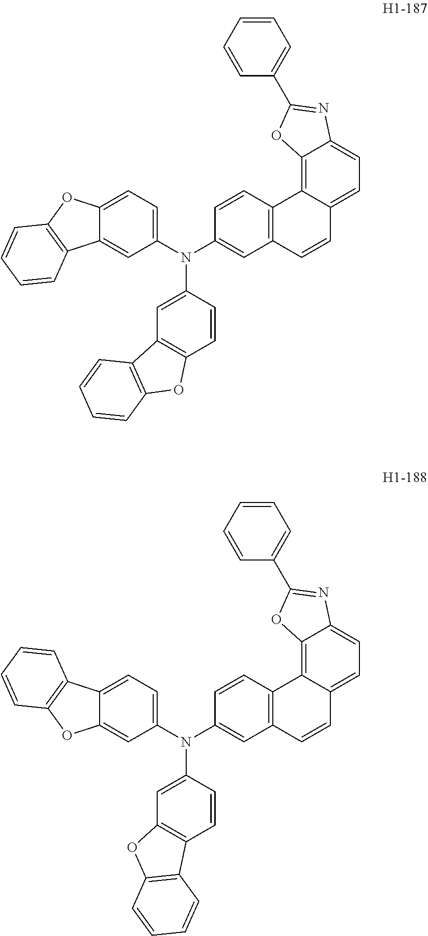 [SMILES: c1ccc(-c2nc3ccc4ccc5cc(N(c6ccc7oc8ccccc8c7c6)c6ccc7oc8ccccc8c7c6)ccc5c4c3o2)cc1 ; c1ccc(-c2nc3ccc4ccc5cc(N(c6ccc7c(c6)oc6ccccc67)c6ccc7c(c6)oc6ccccc67)ccc5c4c3o2)cc1]

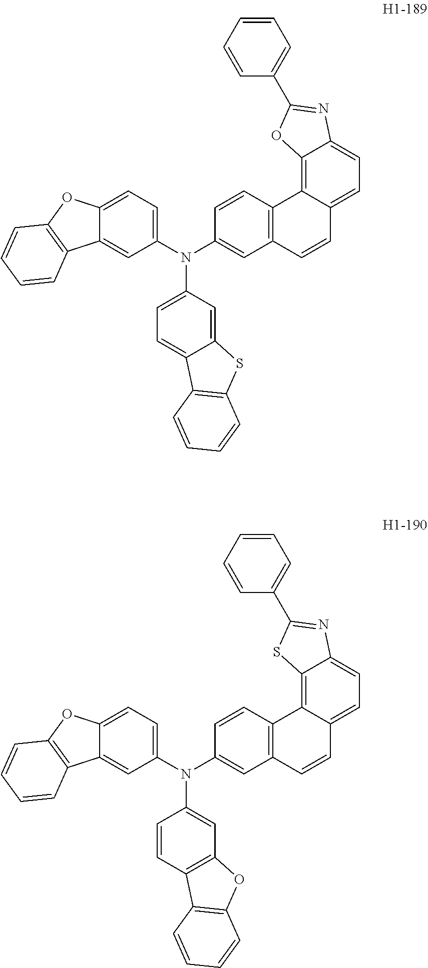 [SMILES: c1ccc(-c2nc3ccc4ccc5cc(N(c6ccc7c(c6)sc6ccccc67)c6ccc7oc8ccccc8c7c6)ccc5c4c3o2)cc1 ; c1ccc(-c2nc3ccc4ccc5cc(N(c6ccc7c(c6)oc6ccccc67)c6ccc7oc8ccccc8c7c6)ccc5c4c3s2)cc1]

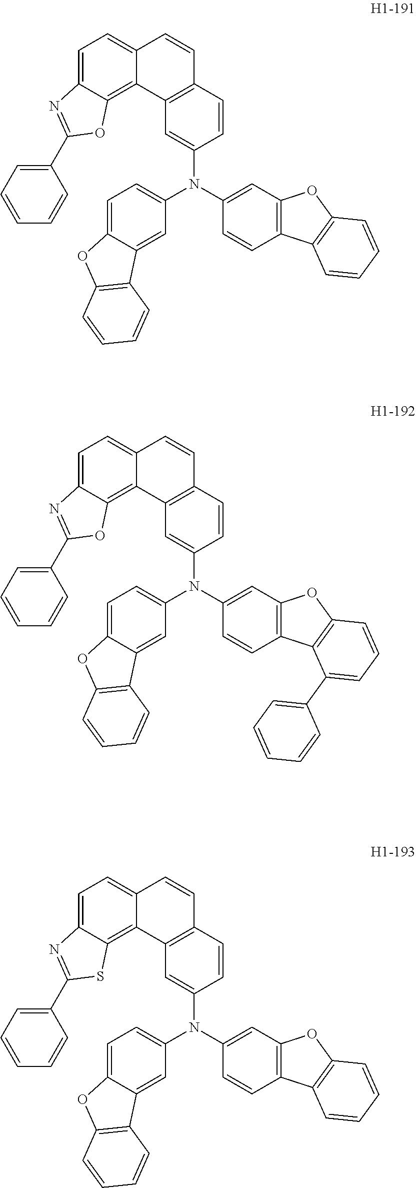 [SMILES: c1ccc(-c2nc3ccc4ccc5ccc(N(c6ccc7c(c6)oc6ccccc67)c6ccc7oc8ccccc8c7c6)cc5c4c3o2)cc1 ; c1ccc(-c2nc3ccc4ccc5ccc(N(c6ccc7c(c6)oc6cccc(-c8ccccc8)c67)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3s2)cc1]

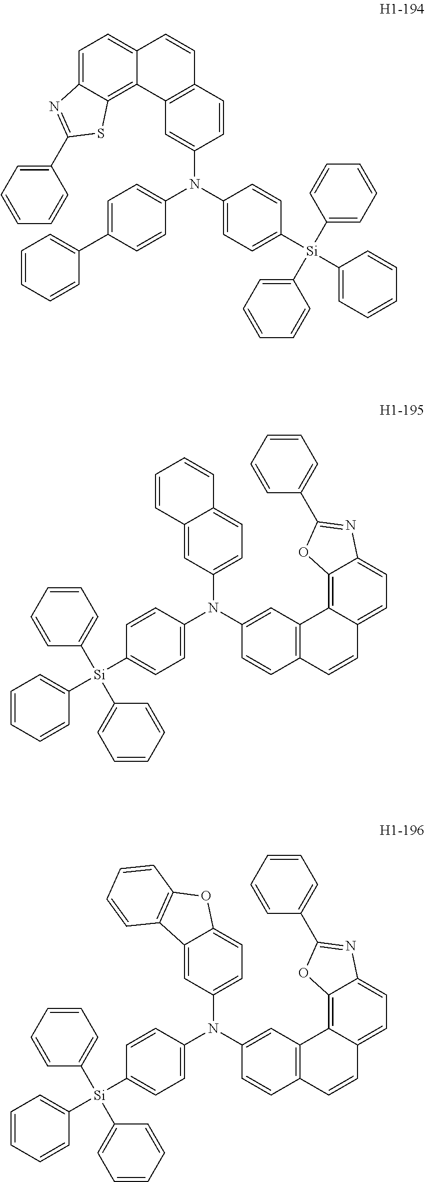 [SMILES: c1ccc(-c2ccc(N(c3ccc([Si](c4ccccc4)(c4ccccc4)c4ccccc4)cc3)c3ccc4ccc5ccc6nc(-c7ccccc7)sc6c5c4c3)cc2)cc1 ; c1ccc(-c2nc3ccc4ccc5ccc(N(c6ccc([Si](c7ccccc7)(c7ccccc7)c7ccccc7)cc6)c6ccc7ccccc7c6)cc5c4c3o2)cc1 ; c1ccc(-c2nc3ccc4ccc5ccc(N(c6ccc([Si](c7ccccc7)(c7ccccc7)c7ccccc7)cc6)c6ccc7oc8ccccc8c7c6)cc5c4c3o2)cc1]

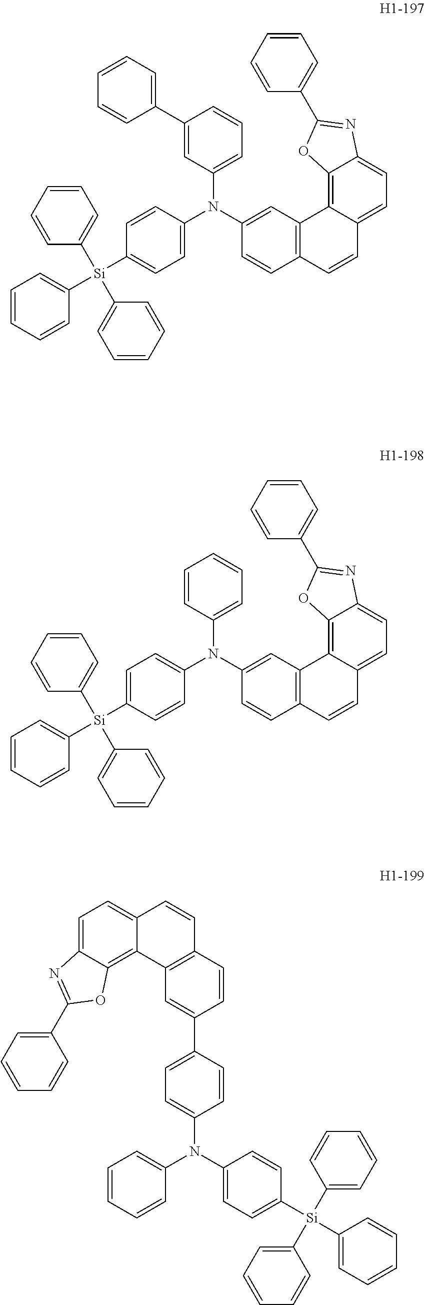 [SMILES: c1ccc(-c2cccc(N(c3ccc([Si](c4ccccc4)(c4ccccc4)c4ccccc4)cc3)c3ccc4ccc5ccc6nc(-c7ccccc7)oc6c5c4c3)c2)cc1 ; c1ccc(-c2nc3ccc4ccc5ccc(N(c6ccccc6)c6ccc([Si](c7ccccc7)(c7ccccc7)c7ccccc7)cc6)cc5c4c3o2)cc1 ; c1ccc(-c2nc3ccc4ccc5ccc(-c6ccc(N(c7ccccc7)c7ccc([Si](c8ccccc8)(c8ccccc8)c8ccccc8)cc7)cc6)cc5c4c3o2)cc1]

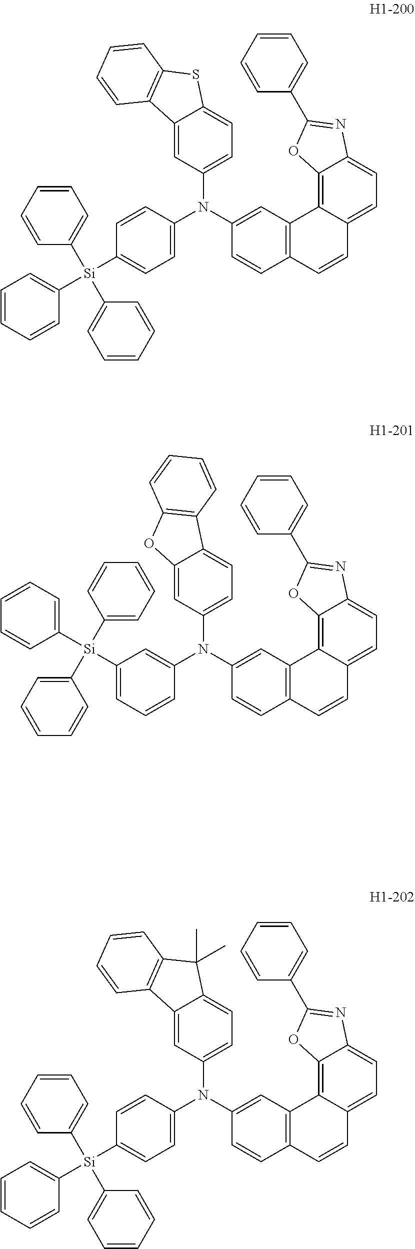 [SMILES: c1ccc(-c2nc3ccc4ccc5ccc(N(c6ccc([Si](c7ccccc7)(c7ccccc7)c7ccccc7)cc6)c6ccc7sc8ccccc8c7c6)cc5c4c3o2)cc1 ; c1ccc(-c2nc3ccc4ccc5ccc(N(c6cccc([Si](c7ccccc7)(c7ccccc7)c7ccccc7)c6)c6ccc7c(c6)oc6ccccc67)cc5c4c3o2)cc1 ; CC1(C)c2ccccc2-c2cc(N(c3ccc([Si](c4ccccc4)(c4ccccc4)c4ccccc4)cc3)c3ccc4ccc5ccc6nc(-c7ccccc7)oc6c5c4c3)ccc21]

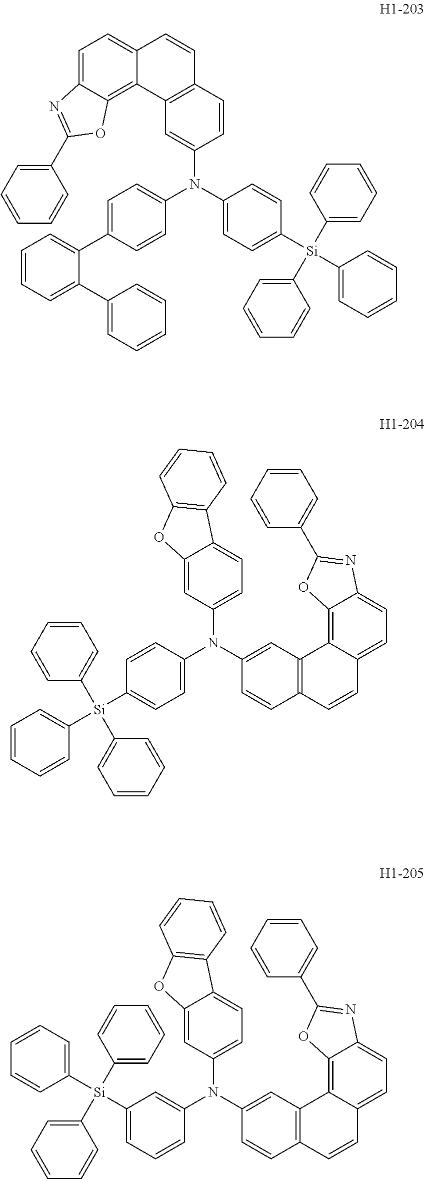 [SMILES: c1ccc(-c2nc3ccc4ccc5ccc(N(c6ccc(-c7ccccc7-c7ccccc7)cc6)c6ccc([Si](c7ccccc7)(c7ccccc7)c7ccccc7)cc6)cc5c4c3o2)cc1 ; c1ccc(-c2nc3ccc4ccc5ccc(N(c6ccc([Si](c7ccccc7)(c7ccccc7)c7ccccc7)cc6)c6ccc7c(c6)oc6ccccc67)cc5c4c3o2)cc1 ; c1ccc(-c2nc3ccc4ccc5ccc(N(c6cccc([Si](c7ccccc7)(c7ccccc7)c7ccccc7)c6)c6ccc7c(c6)oc6ccccc67)cc5c4c3o2)cc1]

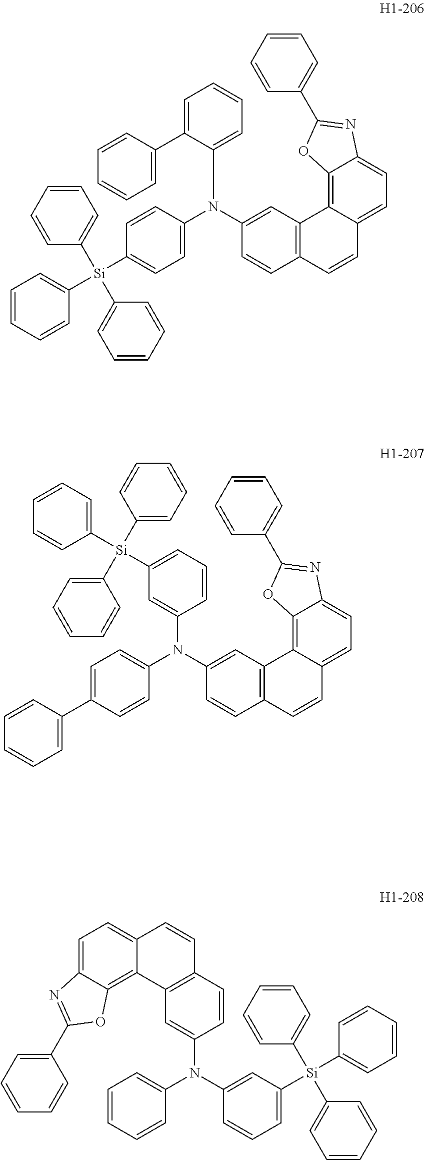 [SMILES: c1ccc(-c2nc3ccc4ccc5ccc(N(c6ccc([Si](c7ccccc7)(c7ccccc7)c7ccccc7)cc6)c6ccccc6-c6ccccc6)cc5c4c3o2)cc1 ; c1ccc(-c2ccc(N(c3cccc([Si](c4ccccc4)(c4ccccc4)c4ccccc4)c3)c3ccc4ccc5ccc6nc(-c7ccccc7)oc6c5c4c3)cc2)cc1 ; c1ccc(-c2nc3ccc4ccc5ccc(N(c6ccccc6)c6cccc([Si](c7ccccc7)(c7ccccc7)c7ccccc7)c6)cc5c4c3o2)cc1]

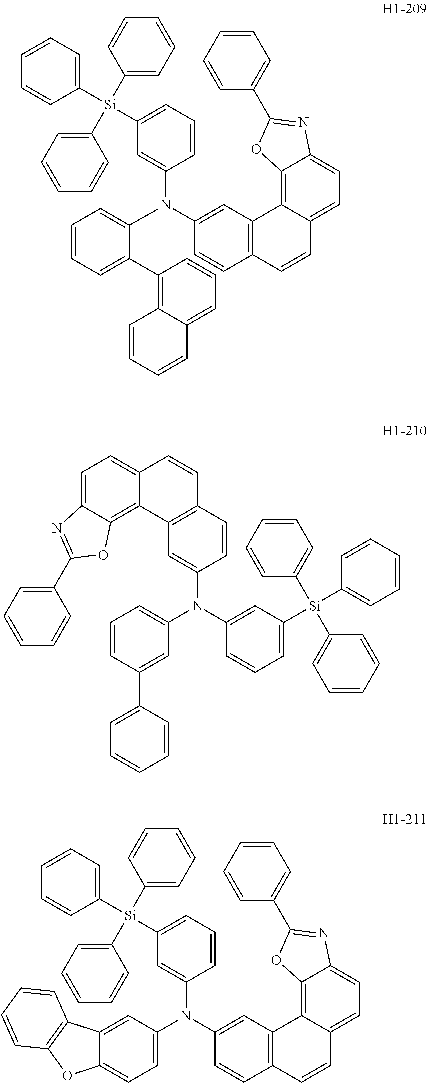 [SMILES: c1ccc(-c2nc3ccc4ccc5ccc(N(c6cccc([Si](c7ccccc7)(c7ccccc7)c7ccccc7)c6)c6ccccc6-c6cccc7ccccc67)cc5c4c3o2)cc1 ; c1ccc(-c2cccc(N(c3cccc([Si](c4ccccc4)(c4ccccc4)c4ccccc4)c3)c3ccc4ccc5ccc6nc(-c7ccccc7)oc6c5c4c3)c2)cc1 ; c1ccc(-c2nc3ccc4ccc5ccc(N(c6cccc([Si](c7ccccc7)(c7ccccc7)c7ccccc7)c6)c6ccc7oc8ccccc8c7c6)cc5c4c3o2)cc1]

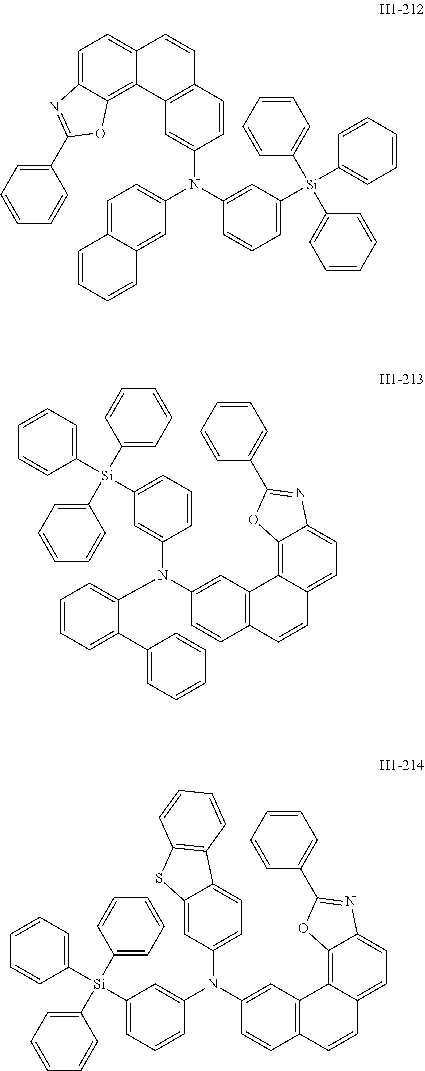 [SMILES: c1ccc(-c2nc3ccc4ccc5ccc(N(c6cccc([Si](c7ccccc7)(c7ccccc7)c7ccccc7)c6)c6ccc7ccccc7c6)cc5c4c3o2)cc1 ; c1ccc(-c2nc3ccc4ccc5ccc(N(c6cccc([Si](c7ccccc7)(c7ccccc7)c7ccccc7)c6)c6ccccc6-c6ccccc6)cc5c4c3o2)cc1 ; c1ccc(-c2nc3ccc4ccc5ccc(N(c6cccc([Si](c7ccccc7)(c7ccccc7)c7ccccc7)c6)c6ccc7c(c6)sc6ccccc67)cc5c4c3o2)cc1]

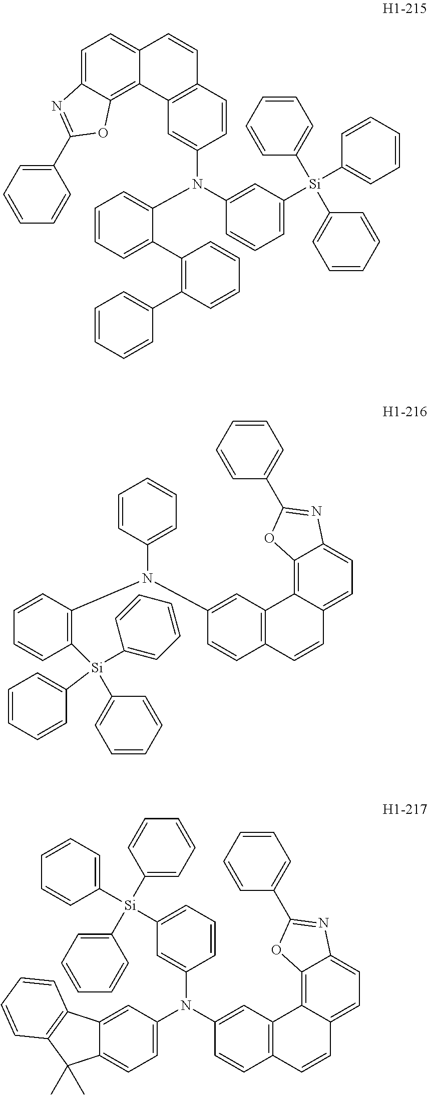 [SMILES: c1ccc(-c2nc3ccc4ccc5ccc(N(c6cccc([Si](c7ccccc7)(c7ccccc7)c7ccccc7)c6)c6ccccc6-c6ccccc6-c6ccccc6)cc5c4c3o2)cc1 ; c1ccc(-c2nc3ccc4ccc5ccc(N(c6ccccc6)c6ccccc6[Si](c6ccccc6)(c6ccccc6)c6ccccc6)cc5c4c3o2)cc1 ; CC1(C)c2ccccc2-c2cc(N(c3cccc([Si](c4ccccc4)(c4ccccc4)c4ccccc4)c3)c3ccc4ccc5ccc6nc(-c7ccccc7)oc6c5c4c3)ccc21]

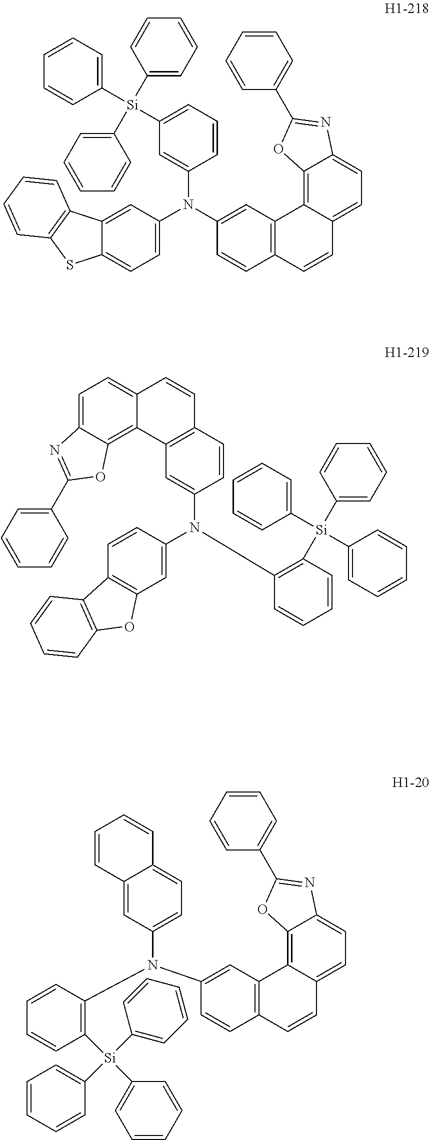 [SMILES: c1ccc(-c2nc3ccc4ccc5ccc(N(c6cccc([Si](c7ccccc7)(c7ccccc7)c7ccccc7)c6)c6ccc7sc8ccccc8c7c6)cc5c4c3o2)cc1 ; c1ccc(-c2nc3ccc4ccc5ccc(N(c6ccc7c(c6)oc6ccccc67)c6ccccc6[Si](c6ccccc6)(c6ccccc6)c6ccccc6)cc5c4c3o2)cc1 ; c1ccc(-c2nc3ccc4ccc5ccc(N(c6ccc7ccccc7c6)c6ccccc6[Si](c6ccccc6)(c6ccccc6)c6ccccc6)cc5c4c3o2)cc1]

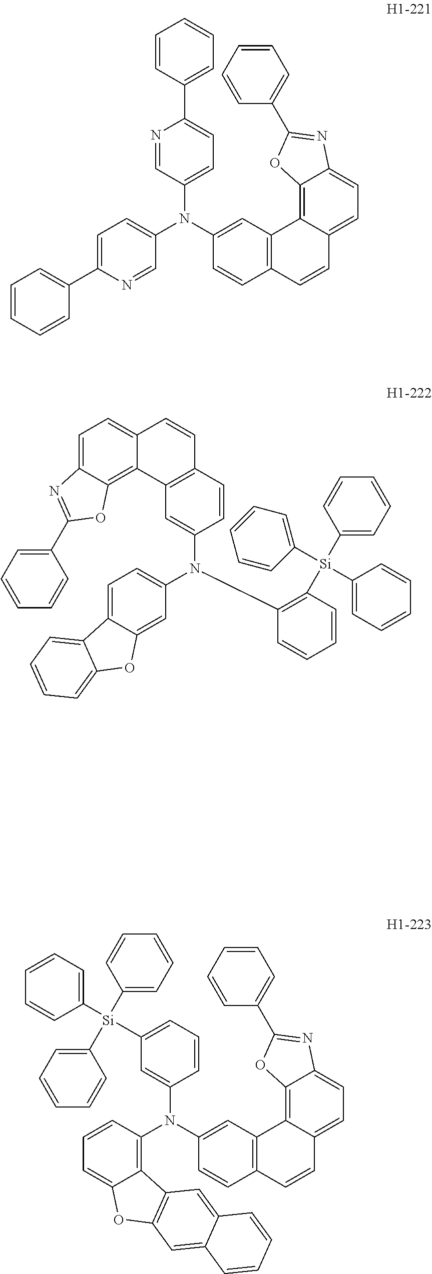 [SMILES: c1ccc(-c2ccc(N(c3ccc(-c4ccccc4)nc3)c3ccc4ccc5ccc6nc(-c7ccccc7)oc6c5c4c3)cn2)cc1 ; c1ccc(-c2nc3ccc4ccc5ccc(N(c6ccc7c(c6)oc6ccccc67)c6ccccc6[Si](c6ccccc6)(c6ccccc6)c6ccccc6)cc5c4c3o2)cc1 ; c1ccc(-c2nc3ccc4ccc5ccc(N(c6cccc([Si](c7ccccc7)(c7ccccc7)c7ccccc7)c6)c6cccc7oc8cc9ccccc9cc8c67)cc5c4c3o2)cc1]

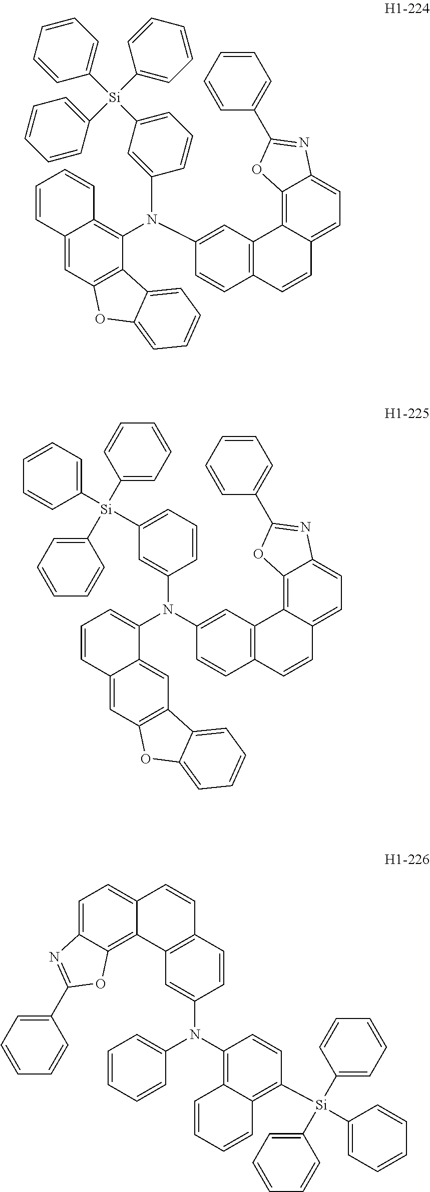 [SMILES: c1ccc(-c2nc3ccc4ccc5ccc(N(c6cccc([Si](c7ccccc7)(c7ccccc7)c7ccccc7)c6)c6c7ccccc7cc7oc8ccccc8c67)cc5c4c3o2)cc1 ; c1ccc(-c2nc3ccc4ccc5ccc(N(c6cccc([Si](c7ccccc7)(c7ccccc7)c7ccccc7)c6)c6cccc7cc8oc9ccccc9c8cc67)cc5c4c3o2)cc1 ; c1ccc(-c2nc3ccc4ccc5ccc(N(c6ccccc6)c6ccc([Si](c7ccccc7)(c7ccccc7)c7ccccc7)c7ccccc67)cc5c4c3o2)cc1]

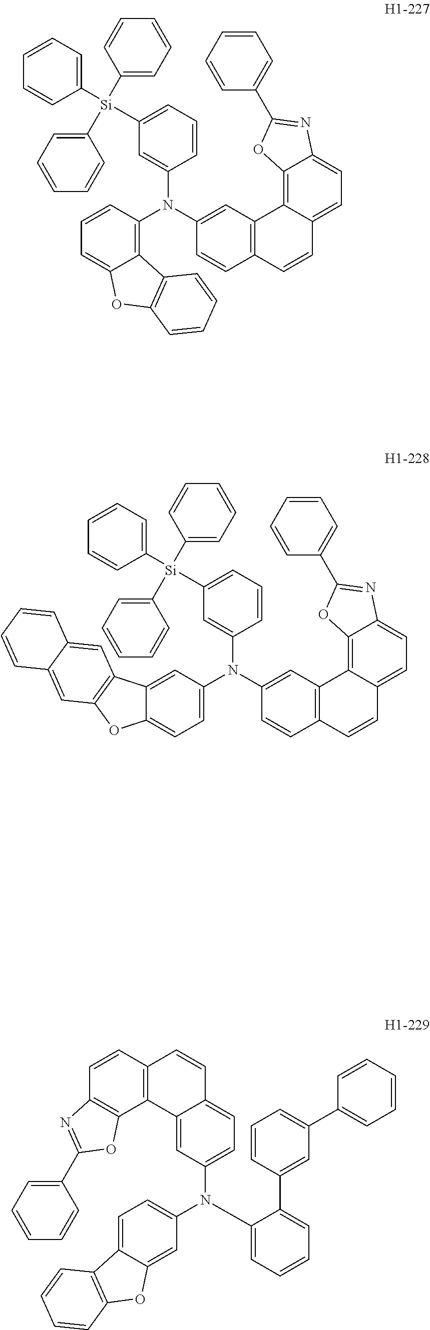 [SMILES: c1ccc(-c2nc3ccc4ccc5ccc(N(c6cccc([Si](c7ccccc7)(c7ccccc7)c7ccccc7)c6)c6cccc7oc8ccccc8c67)cc5c4c3o2)cc1 ; c1ccc(-c2nc3ccc4ccc5ccc(N(c6cccc([Si](c7ccccc7)(c7ccccc7)c7ccccc7)c6)c6ccc7oc8cc9ccccc9cc8c7c6)cc5c4c3o2)cc1 ; c1ccc(-c2cccc(-c3ccccc3N(c3ccc4c(c3)oc3ccccc34)c3ccc4ccc5ccc6nc(-c7ccccc7)oc6c5c4c3)c2)cc1]

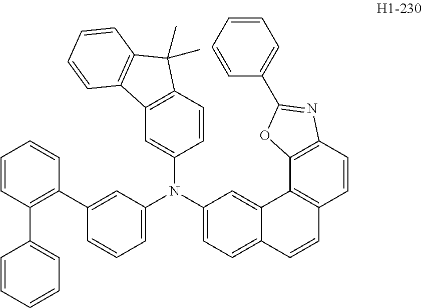 [SMILES: CC1(C)c2ccccc2-c2cc(N(c3cccc(-c4ccccc4-c4ccccc4)c3)c3ccc4ccc5ccc6nc(-c7ccccc7)oc6c5c4c3)ccc21]

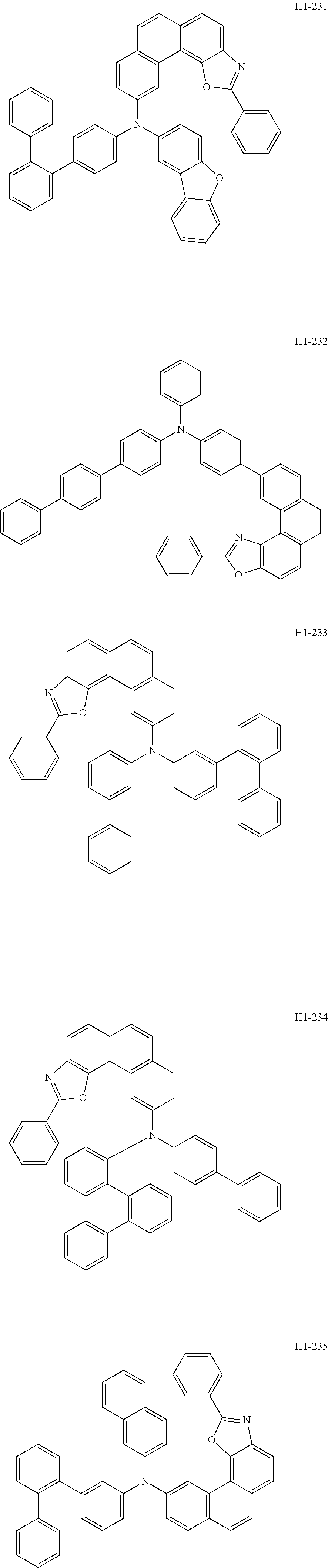 [SMILES: c1ccc(-c2nc3ccc4ccc5ccc(N(c6ccc(-c7ccccc7-c7ccccc7)cc6)c6ccc7oc8ccccc8c7c6)cc5c4c3o2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2cccc(N(c3cccc(-c4ccccc4-c4ccccc4)c3)c3ccc4ccc5ccc6nc(-c7ccccc7)oc6c5c4c3)c2)cc1 ; c1ccc(-c2ccc(N(c3ccc4ccc5ccc6nc(-c7ccccc7)oc6c5c4c3)c3ccccc3-c3ccccc3-c3ccccc3)cc2)cc1 ; c1ccc(-c2nc3ccc4ccc5ccc(N(c6cccc(-c7ccccc7-c7ccccc7)c6)c6ccc7ccccc7c6)cc5c4c3o2)cc1]

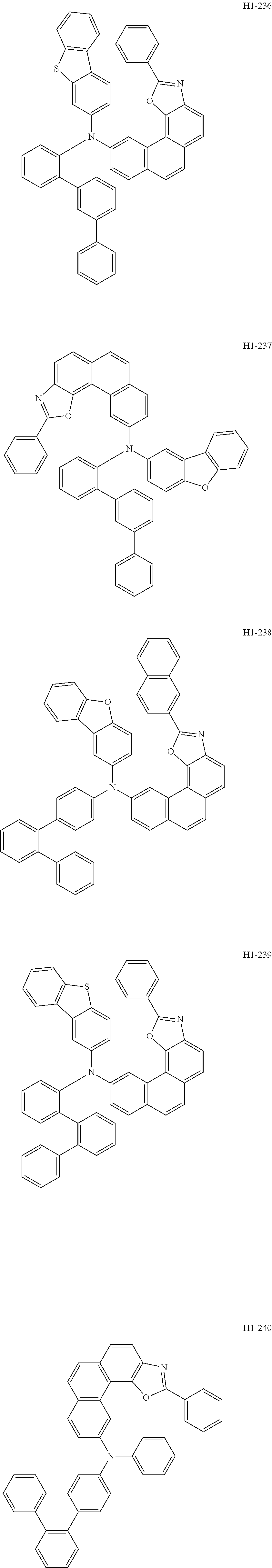 [SMILES: c1ccc(-c2cccc(-c3ccccc3N(c3ccc4c(c3)sc3ccccc34)c3ccc4ccc5ccc6nc(-c7ccccc7)oc6c5c4c3)c2)cc1 ; c1ccc(-c2cccc(-c3ccccc3N(c3ccc4oc5ccccc5c4c3)c3ccc4ccc5ccc6nc(-c7ccccc7)oc6c5c4c3)c2)cc1 ; c1ccc(-c2ccccc2-c2ccc(N(c3ccc4oc5ccccc5c4c3)c3ccc4ccc5ccc6nc(-c7ccc8ccccc8c7)oc6c5c4c3)cc2)cc1 ; c1ccc(-c2nc3ccc4ccc5ccc(N(c6ccc7sc8ccccc8c7c6)c6ccccc6-c6ccccc6-c6ccccc6)cc5c4c3o2)cc1 ; c1ccc(-c2nc3ccc4ccc5ccc(N(c6ccccc6)c6ccc(-c7ccccc7-c7ccccc7)cc6)cc5c4c3o2)cc1]

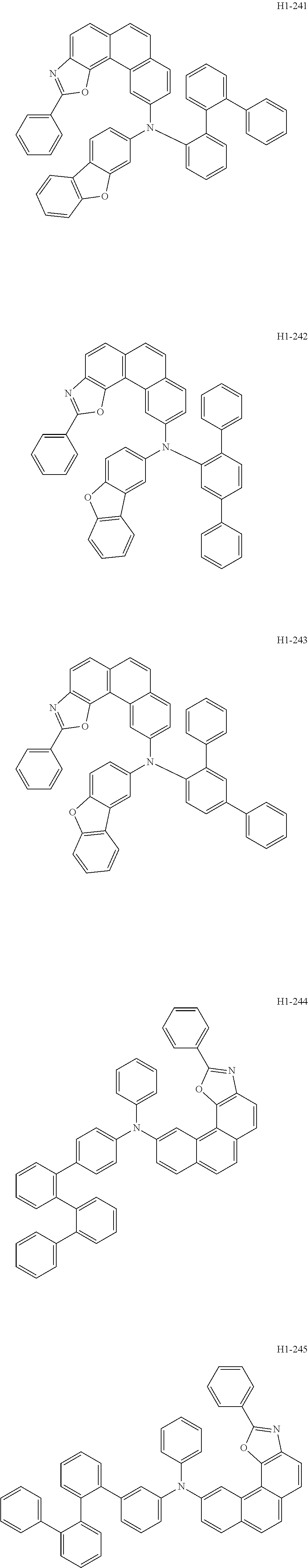 [SMILES: c1ccc(-c2nc3ccc4ccc5ccc(N(c6ccc7c(c6)oc6ccccc67)c6ccccc6-c6ccccc6-c6ccccc6)cc5c4c3o2)cc1 ; c1ccc(-c2ccc(-c3ccccc3)c(N(c3ccc4oc5ccccc5c4c3)c3ccc4ccc5ccc6nc(-c7ccccc7)oc6c5c4c3)c2)cc1 ; c1ccc(-c2ccc(N(c3ccc4oc5ccccc5c4c3)c3ccc4ccc5ccc6nc(-c7ccccc7)oc6c5c4c3)c(-c3ccccc3)c2)cc1 ; c1ccc(-c2nc3ccc4ccc5ccc(N(c6ccccc6)c6ccc(-c7ccccc7-c7ccccc7-c7ccccc7)cc6)cc5c4c3o2)cc1 ; c1ccc(-c2nc3ccc4ccc5ccc(N(c6ccccc6)c6cccc(-c7ccccc7-c7ccccc7-c7ccccc7)c6)cc5c4c3o2)cc1]

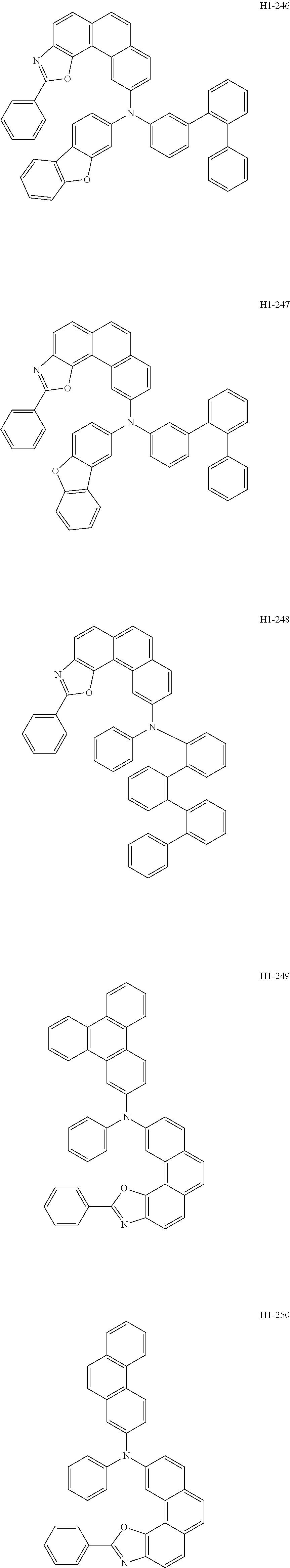 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1 ; c1ccc(-c2nc3ccc4ccc5ccc(N(c6cccc(-c7ccccc7-c7ccccc7)c6)c6ccc7oc8ccccc8c7c6)cc5c4c3o2)cc1 ; c1ccc(-c2nc3ccc4ccc5ccc(N(c6ccccc6)c6ccccc6-c6ccccc6-c6ccccc6-c6ccccc6)cc5c4c3o2)cc1 ; c1ccc(-c2nc3ccc4ccc5ccc(N(c6ccccc6)c6ccc7c8ccccc8c8ccccc8c7c6)cc5c4c3o2)cc1 ; c1ccc(-c2nc3ccc4ccc5ccc(N(c6ccccc6)c6ccc7c(ccc8ccccc87)c6)cc5c4c3o2)cc1]

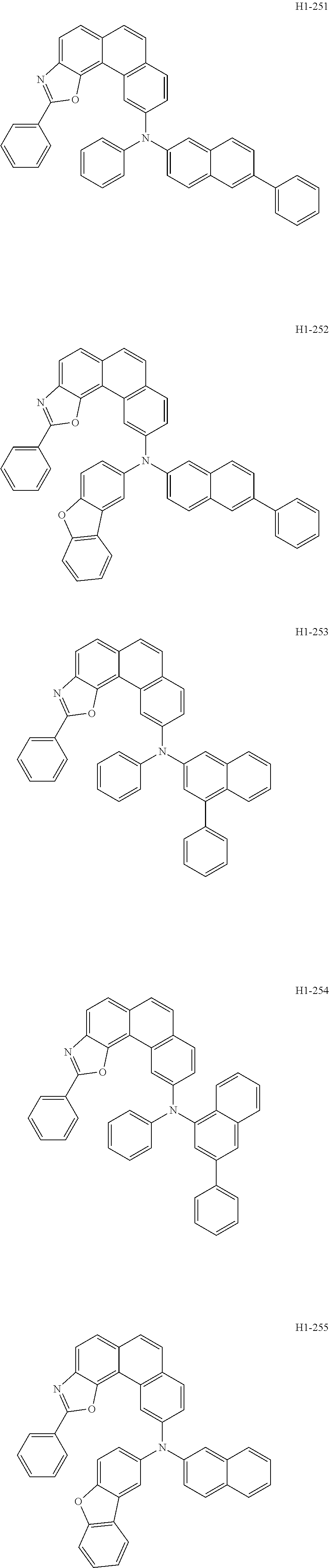 [SMILES: c1ccc(-c2ccc3cc(N(c4ccccc4)c4ccc5ccc6ccc7nc(-c8ccccc8)oc7c6c5c4)ccc3c2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1 ; c1ccc(-c2nc3ccc4ccc5ccc(N(c6ccccc6)c6cc(-c7ccccc7)c7ccccc7c6)cc5c4c3o2)cc1 ; c1ccc(-c2cc(N(c3ccccc3)c3ccc4ccc5ccc6nc(-c7ccccc7)oc6c5c4c3)c3ccccc3c2)cc1 ; c1ccc(-c2nc3ccc4ccc5ccc(N(c6ccc7ccccc7c6)c6ccc7oc8ccccc8c7c6)cc5c4c3o2)cc1]

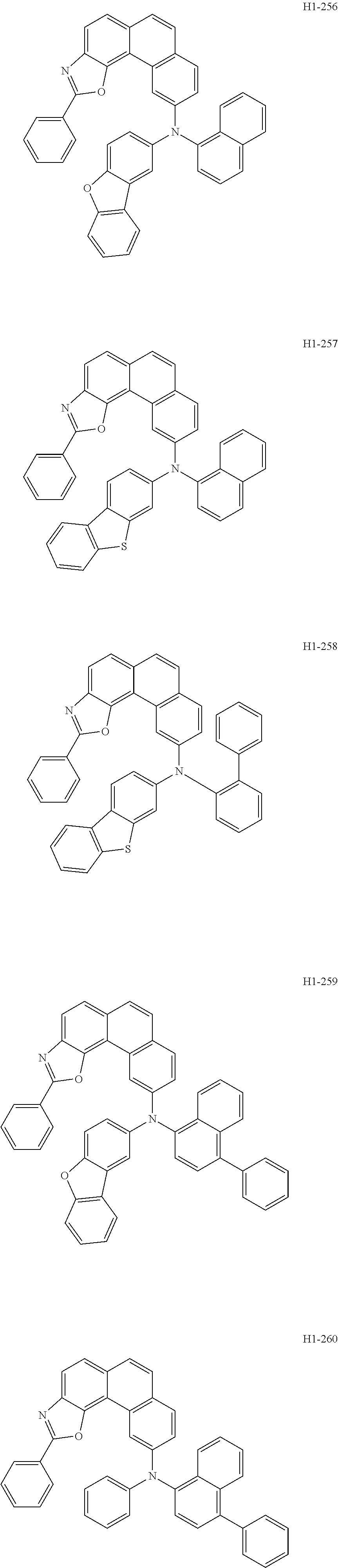 [SMILES: c1ccc(-c2nc3ccc4ccc5ccc(N(c6ccc7oc8ccccc8c7c6)c6cccc7ccccc67)cc5c4c3o2)cc1 ; c1ccc(-c2nc3ccc4ccc5ccc(N(c6ccc7c(c6)sc6ccccc67)c6cccc7ccccc67)cc5c4c3o2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(N(c6ccc7oc8ccccc8c7c6)c6ccc(-c7ccccc7)c7ccccc67)cc5c4c3o2)cc1 ; c1ccc(-c2nc3ccc4ccc5ccc(N(c6ccccc6)c6ccc(-c7ccccc7)c7ccccc67)cc5c4c3o2)cc1]

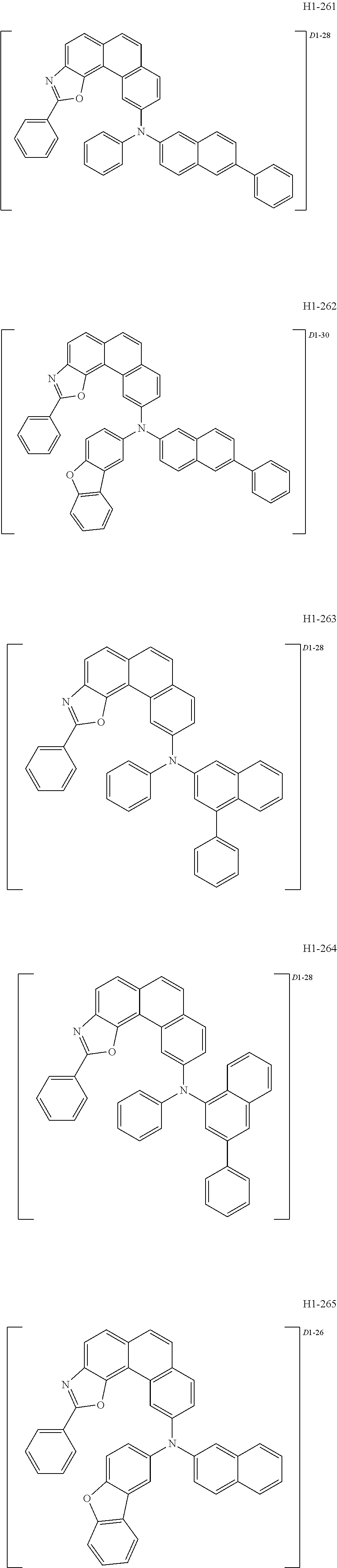

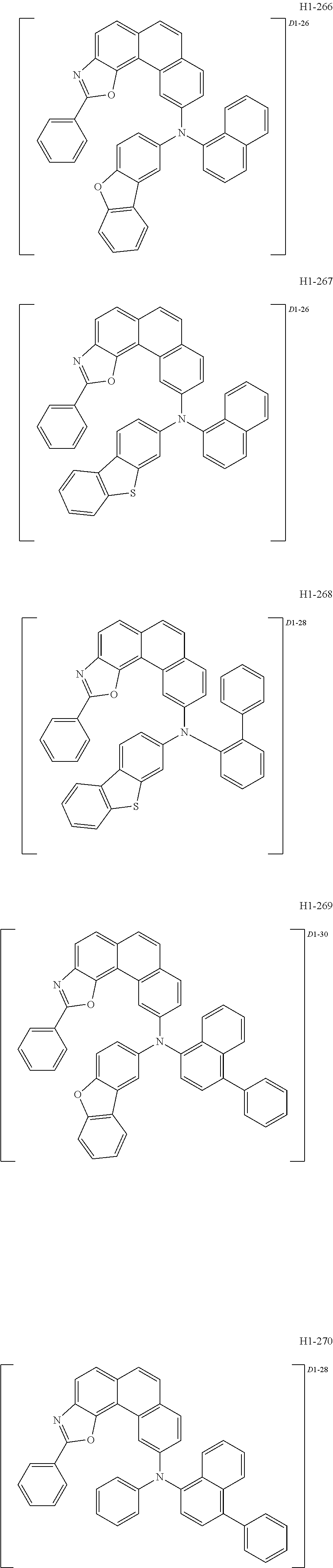

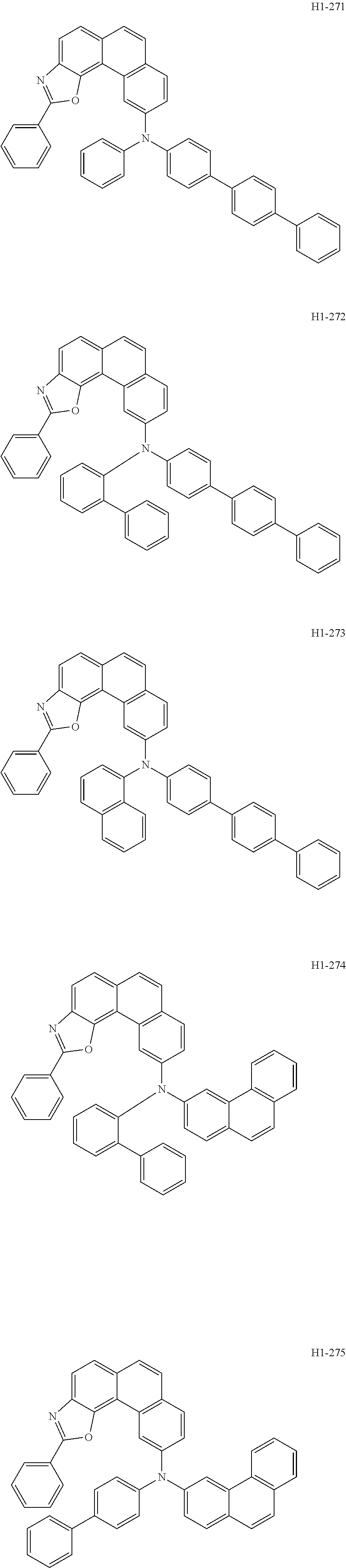 [SMILES: c1ccc(-c2ccc(-c3ccc(N(c4ccccc4)c4ccc5ccc6ccc7nc(-c8ccccc8)oc7c6c5c4)cc3)cc2)cc1 ; c1ccc(-c2ccc(-c3ccc(N(c4ccc5ccc6ccc7nc(-c8ccccc8)oc7c6c5c4)c4ccccc4-c4ccccc4)cc3)cc2)cc1 ; c1ccc(-c2ccc(-c3ccc(N(c4ccc5ccc6ccc7nc(-c8ccccc8)oc7c6c5c4)c4cccc5ccccc45)cc3)cc2)cc1 ; c1ccc(-c2nc3ccc4ccc5ccc(N(c6ccc7ccc8ccccc8c7c6)c6ccccc6-c6ccccc6)cc5c4c3o2)cc1 ; c1ccc(-c2ccc(N(c3ccc4ccc5ccccc5c4c3)c3ccc4ccc5ccc6nc(-c7ccccc7)oc6c5c4c3)cc2)cc1]

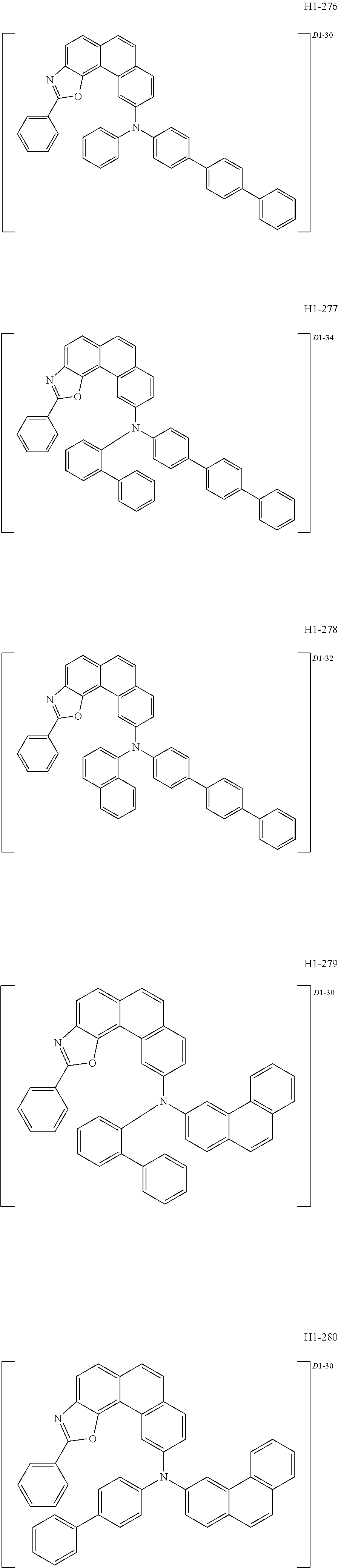

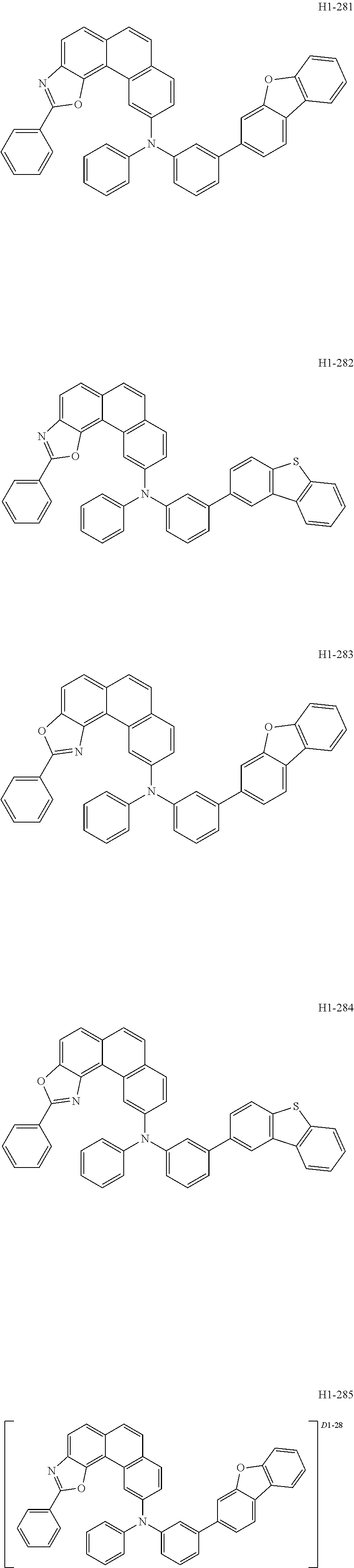

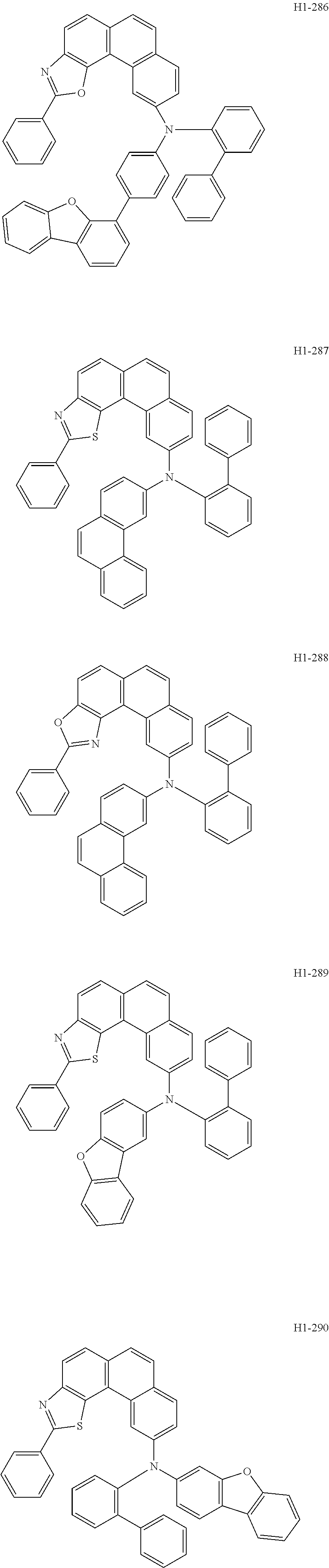 [SMILES: c1ccc(-c2nc3ccc4ccc5ccc(N(c6ccc(-c7cccc8c7oc7ccccc78)cc6)c6ccccc6-c6ccccc6)cc5c4c3o2)cc1 ; c1ccc(-c2nc3ccc4ccc5ccc(N(c6ccc7ccc8ccccc8c7c6)c6ccccc6-c6ccccc6)cc5c4c3s2)cc1 ; c1ccc(-c2nc3c(ccc4ccc5ccc(N(c6ccc7ccc8ccccc8c7c6)c6ccccc6-c6ccccc6)cc5c43)o2)cc1 ; c1ccc(-c2nc3ccc4ccc5ccc(N(c6ccc7oc8ccccc8c7c6)c6ccccc6-c6ccccc6)cc5c4c3s2)cc1 ; c1ccc(-c2nc3ccc4ccc5ccc(N(c6ccc7c(c6)oc6ccccc67)c6ccccc6-c6ccccc6)cc5c4c3s2)cc1]

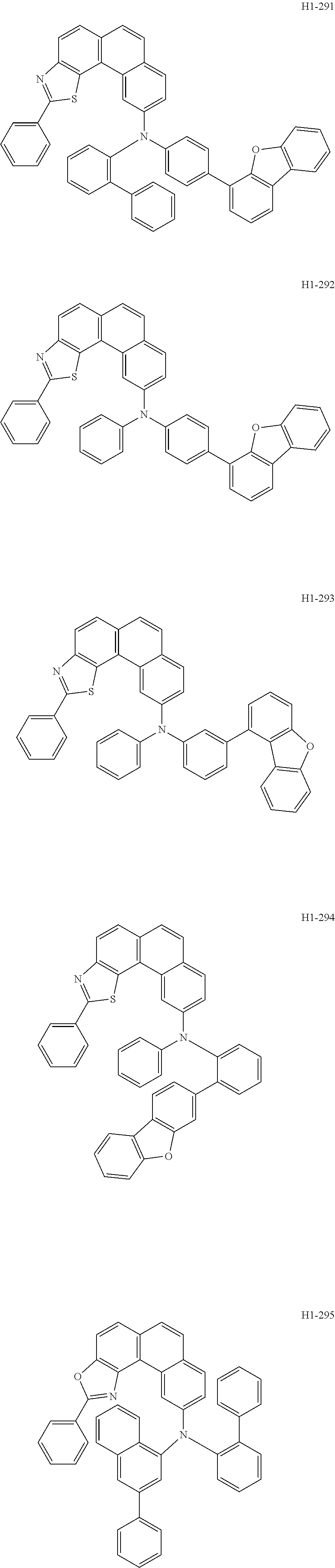 [SMILES: c1ccc(-c2nc3ccc4ccc5ccc(N(c6ccc(-c7cccc8c7oc7ccccc78)cc6)c6ccccc6-c6ccccc6)cc5c4c3s2)cc1 ; c1ccc(-c2nc3ccc4ccc5ccc(N(c6ccccc6)c6ccc(-c7cccc8c7oc7ccccc78)cc6)cc5c4c3s2)cc1 ; c1ccc(-c2nc3ccc4ccc5ccc(N(c6ccccc6)c6cccc(-c7cccc8oc9ccccc9c78)c6)cc5c4c3s2)cc1 ; c1ccc(-c2nc3ccc4ccc5ccc(N(c6ccccc6)c6ccccc6-c6ccc7c(c6)oc6ccccc67)cc5c4c3s2)cc1 ; c1ccc(-c2cc(N(c3ccc4ccc5ccc6oc(-c7ccccc7)nc6c5c4c3)c3ccccc3-c3ccccc3)c3ccccc3c2)cc1]

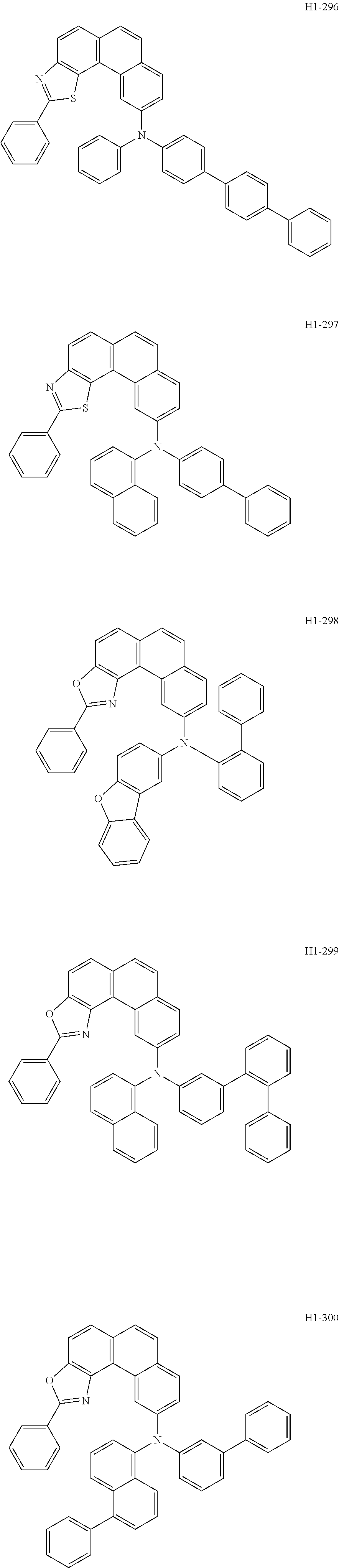 [SMILES: c1ccc(-c2ccc(-c3ccc(N(c4ccccc4)c4ccc5ccc6ccc7nc(-c8ccccc8)sc7c6c5c4)cc3)cc2)cc1 ; c1ccc(-c2ccc(N(c3ccc4ccc5ccc6nc(-c7ccccc7)sc6c5c4c3)c3cccc4ccccc34)cc2)cc1 ; c1ccc(-c2nc3c(ccc4ccc5ccc(N(c6ccc7oc8ccccc8c7c6)c6ccccc6-c6ccccc6)cc5c43)o2)cc1 ; c1ccc(-c2nc3c(ccc4ccc5ccc(N(c6cccc(-c7ccccc7-c7ccccc7)c6)c6cccc7ccccc67)cc5c43)o2)cc1 ; c1ccc(-c2cccc(N(c3ccc4ccc5ccc6oc(-c7ccccc7)nc6c5c4c3)c3cccc4c(-c5ccccc5)cccc34)c2)cc1]

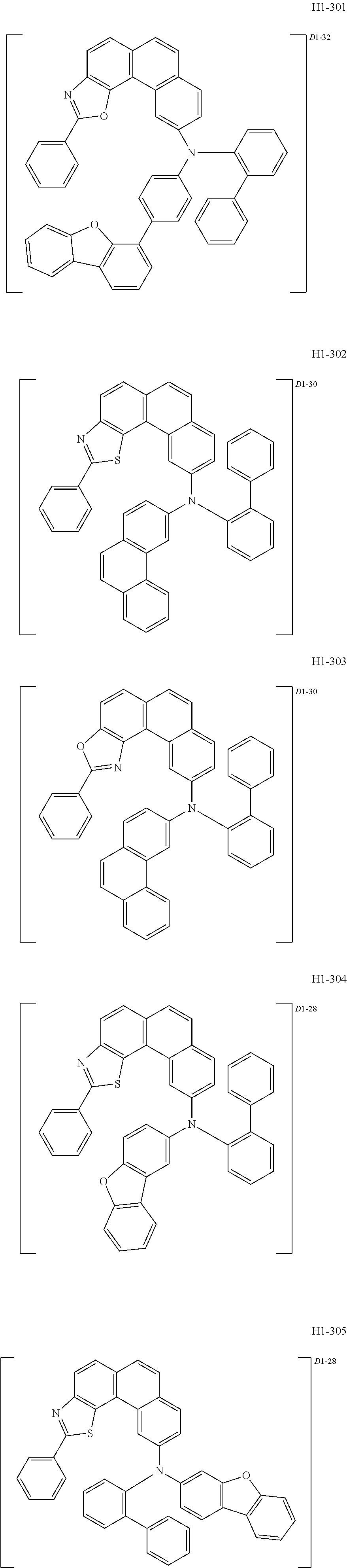

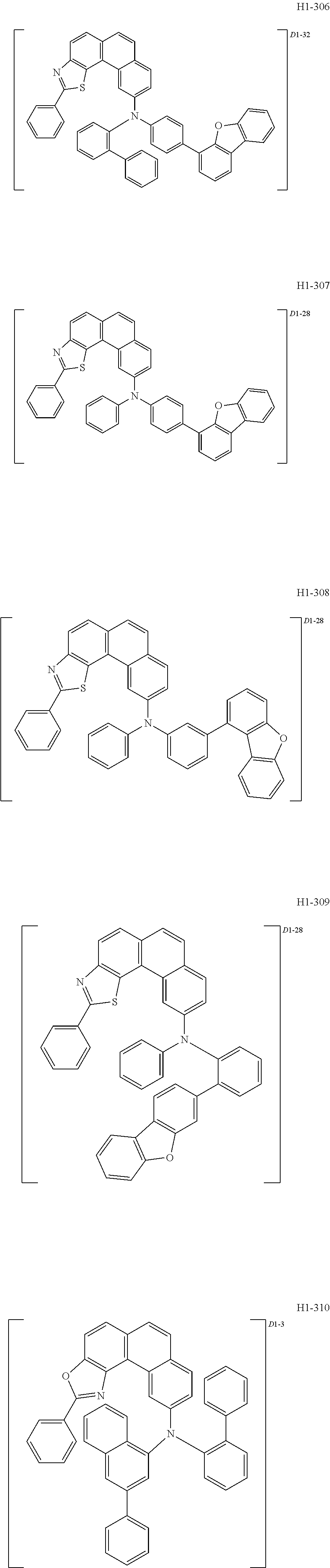

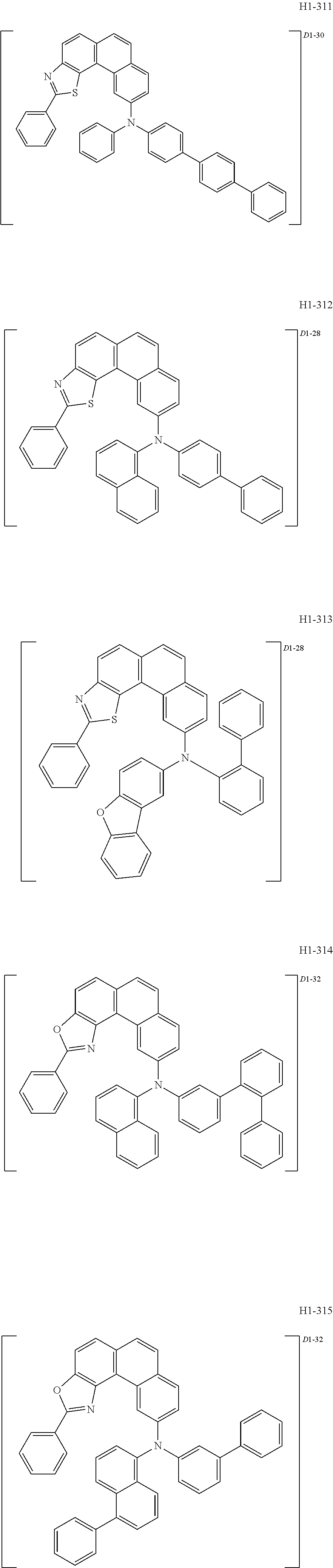

The compound represented by formula 2 may be selected from the group consisting of the following compounds, but is not limited thereto.

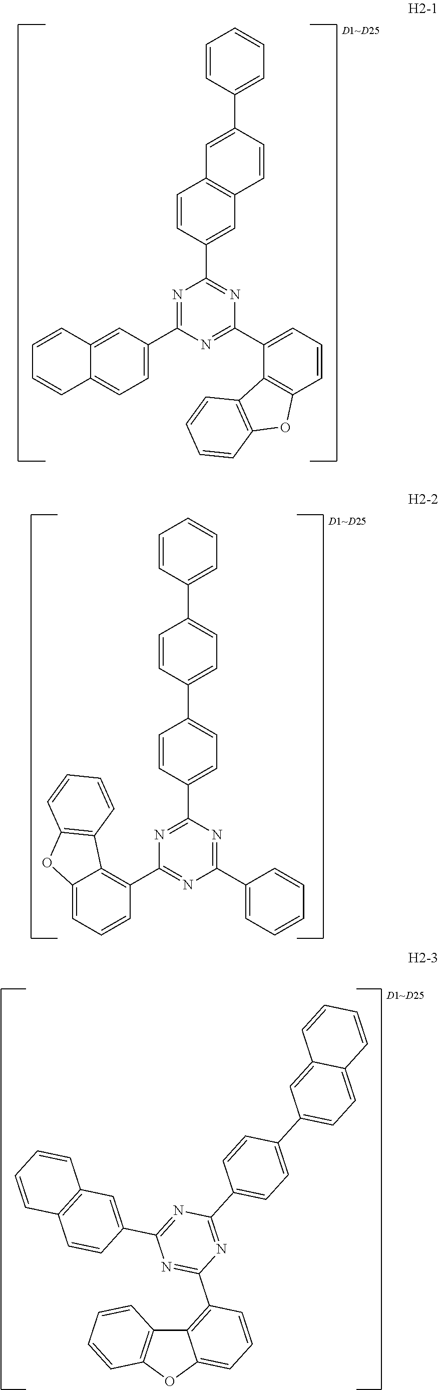

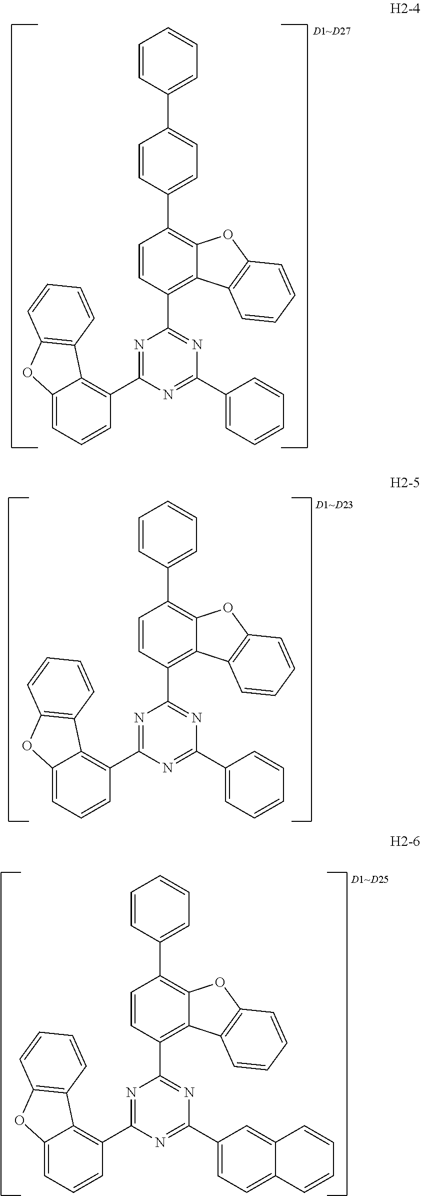

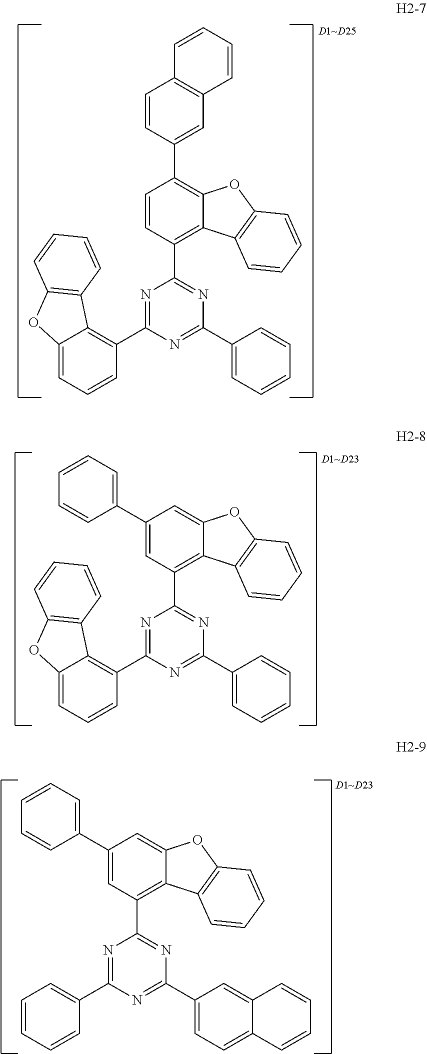

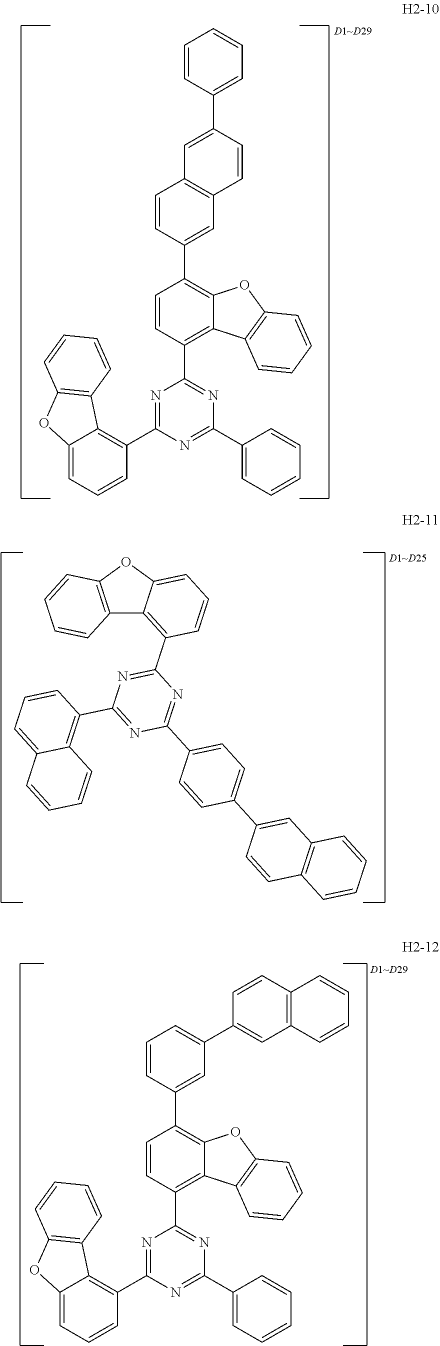

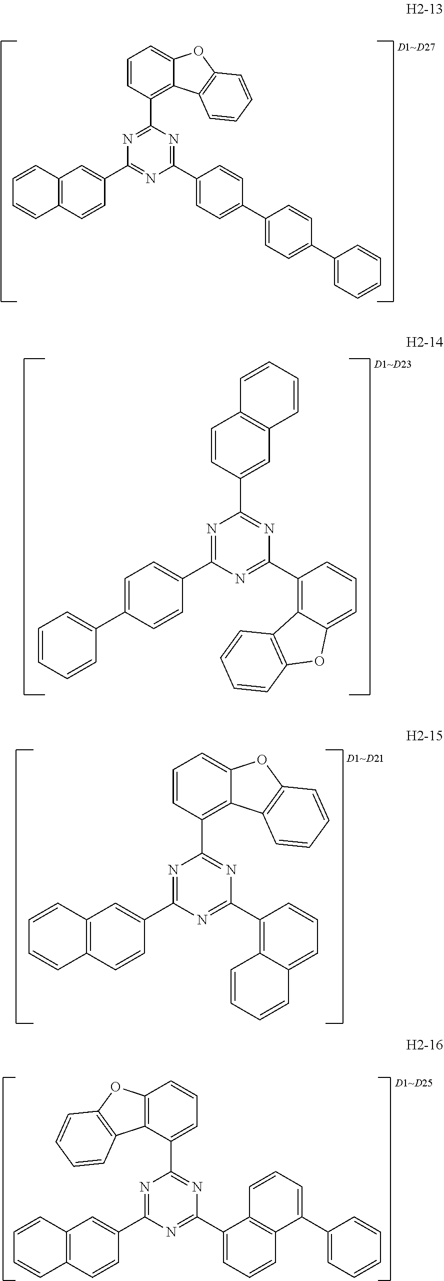

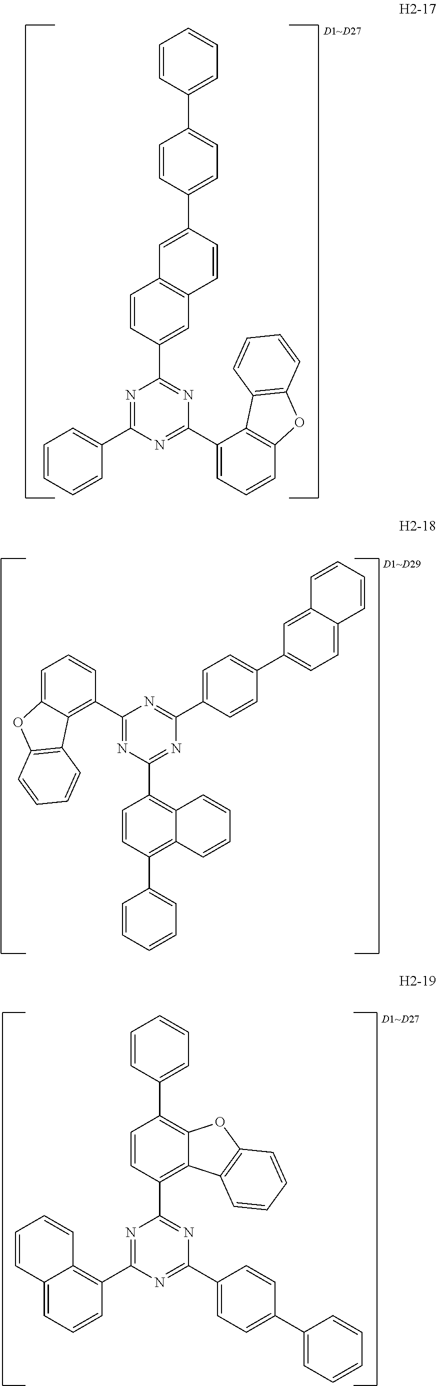

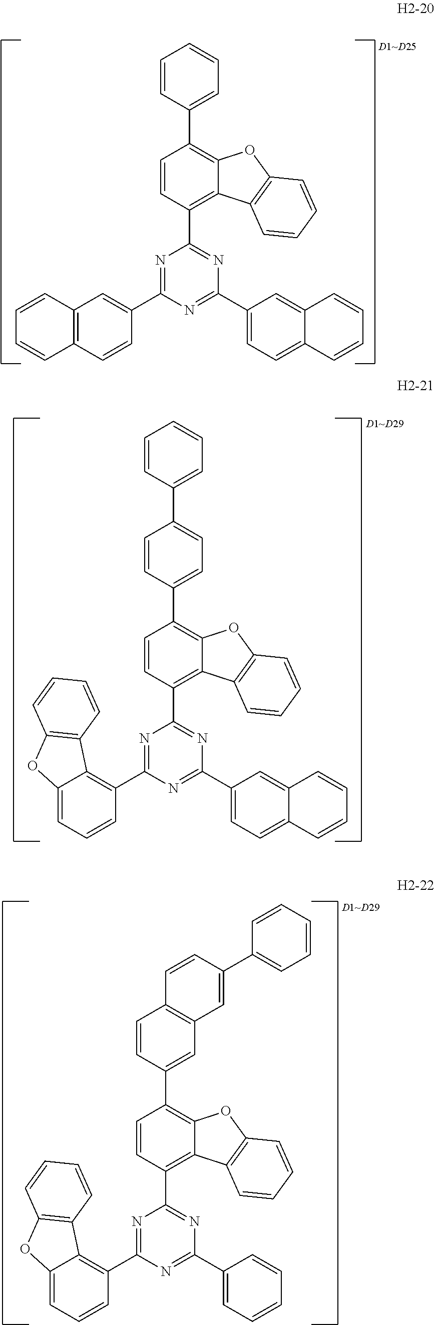

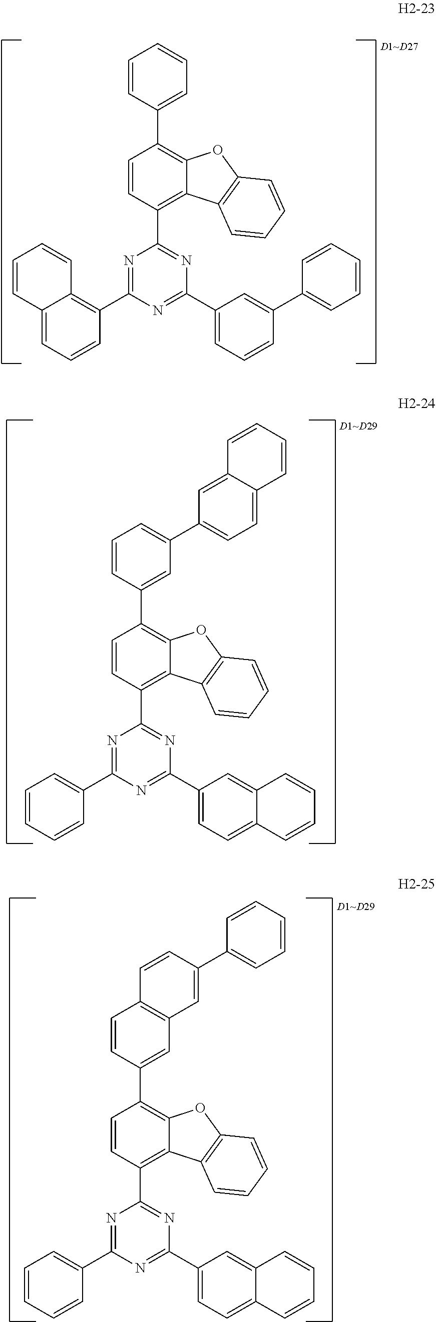

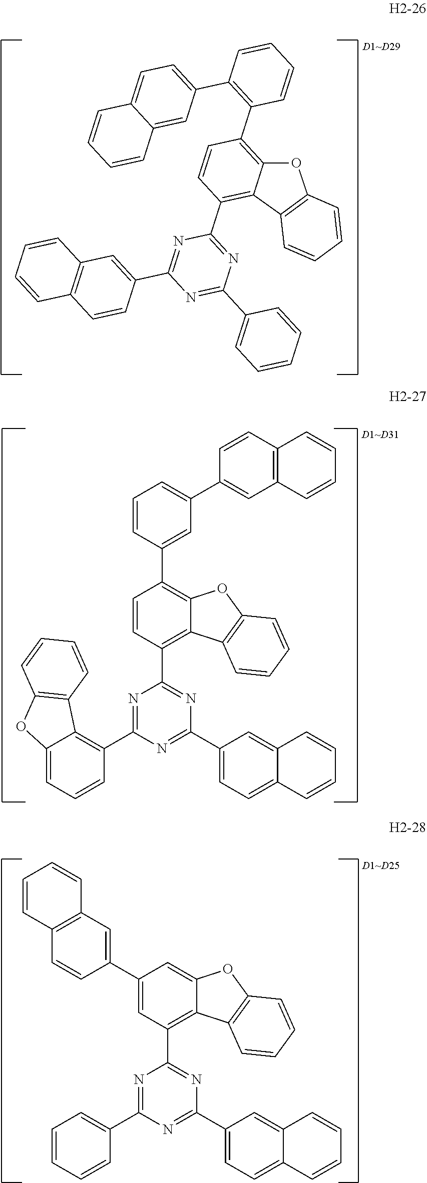

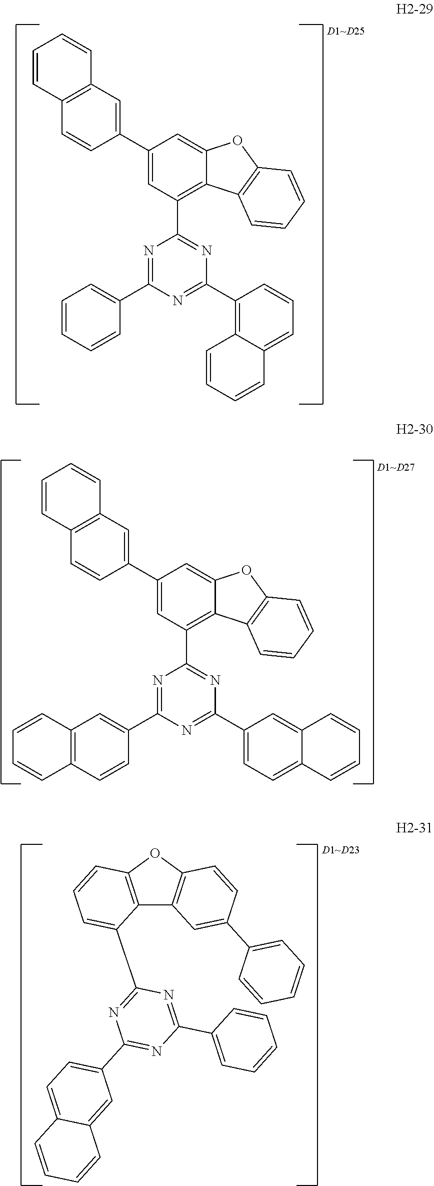

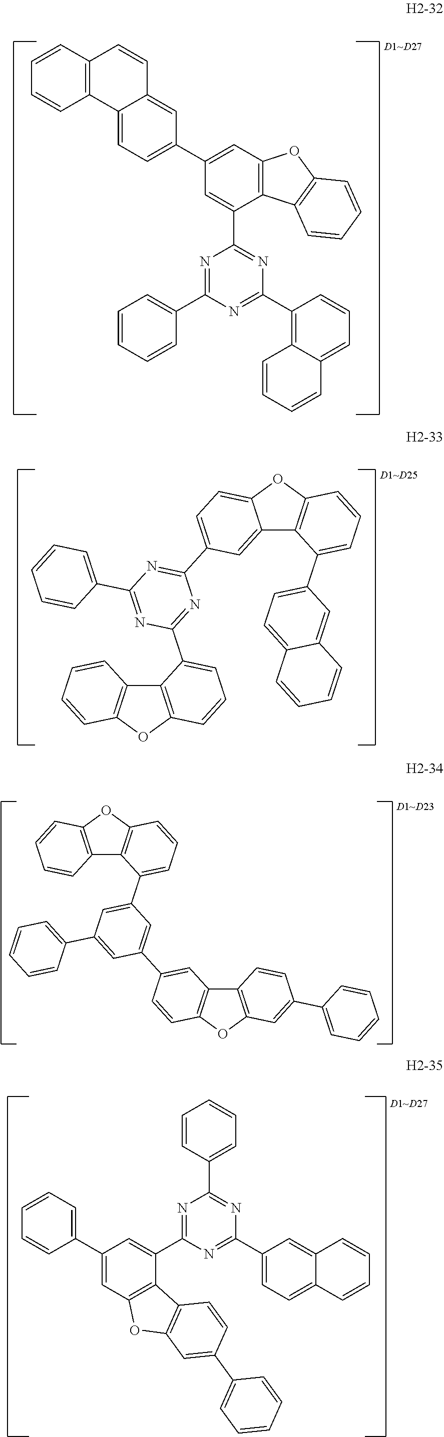

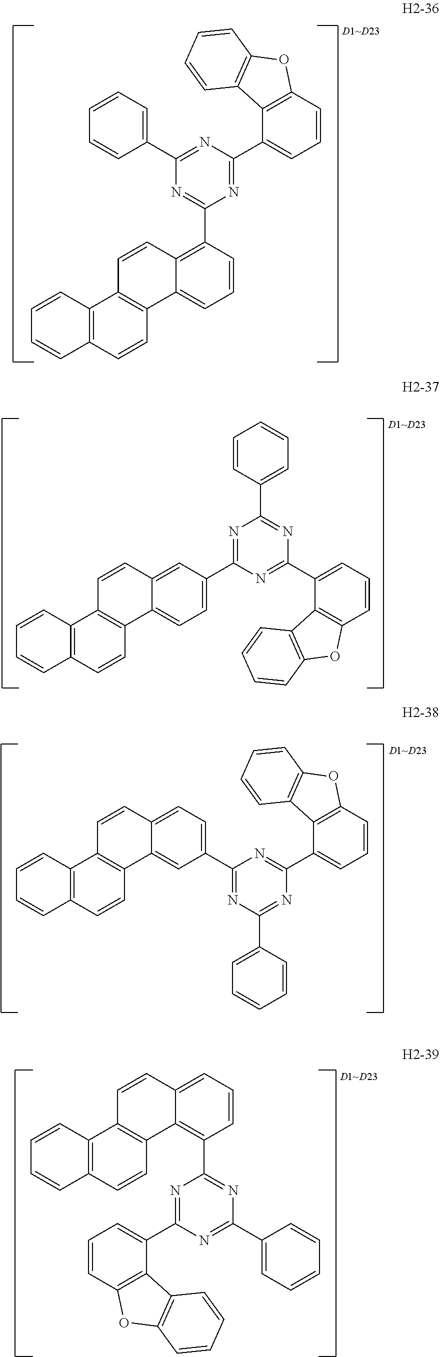

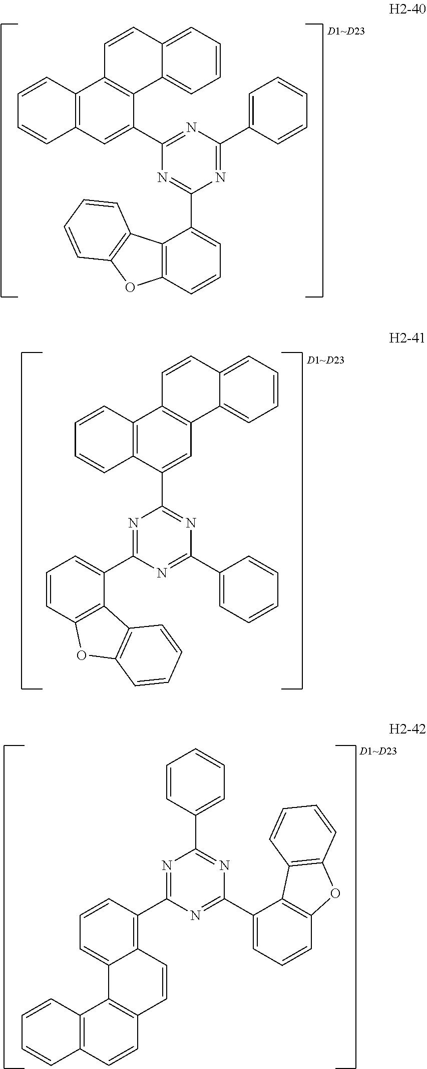

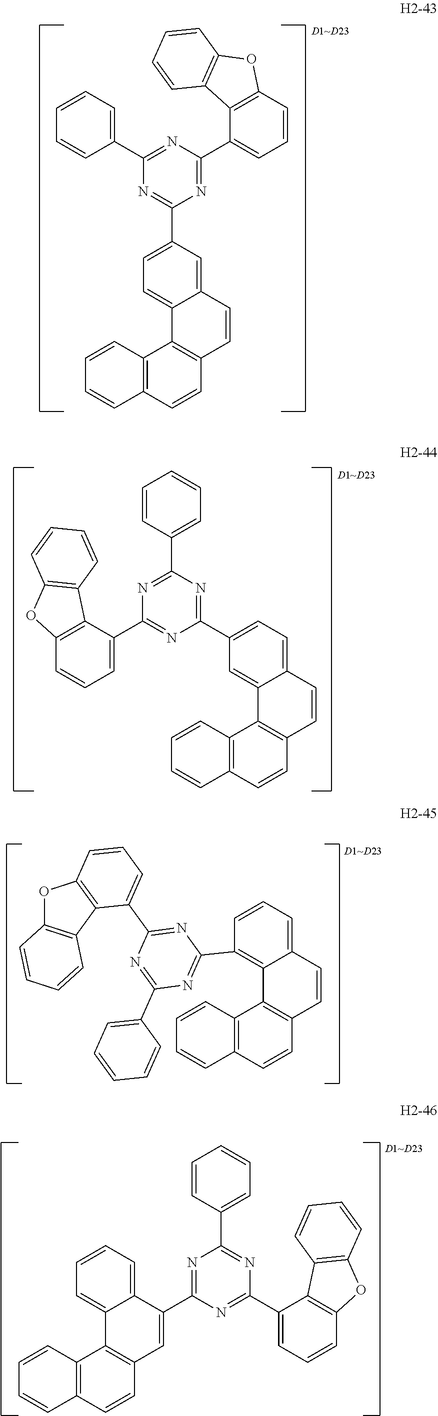

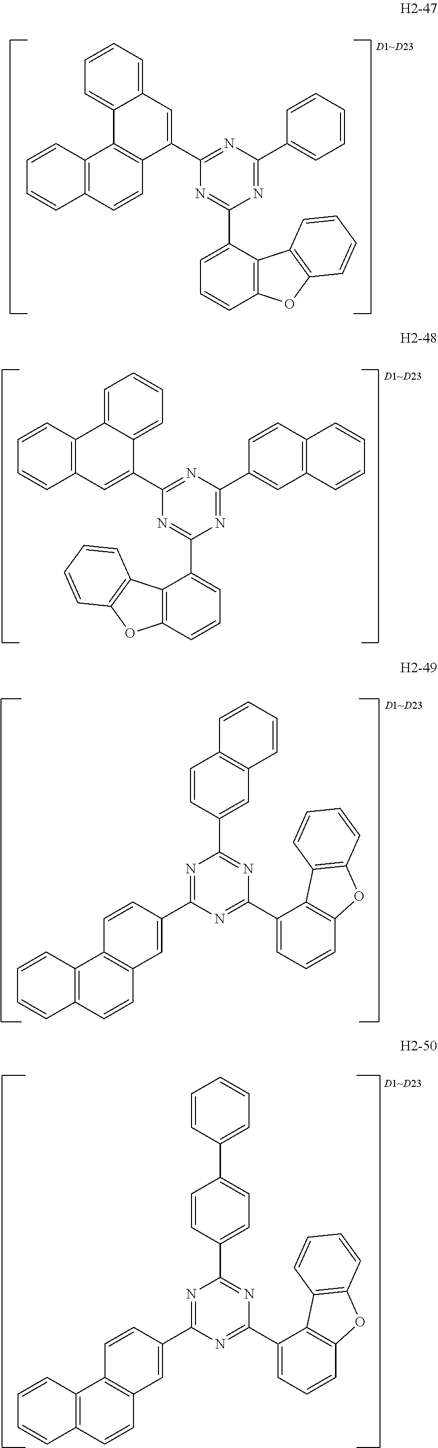

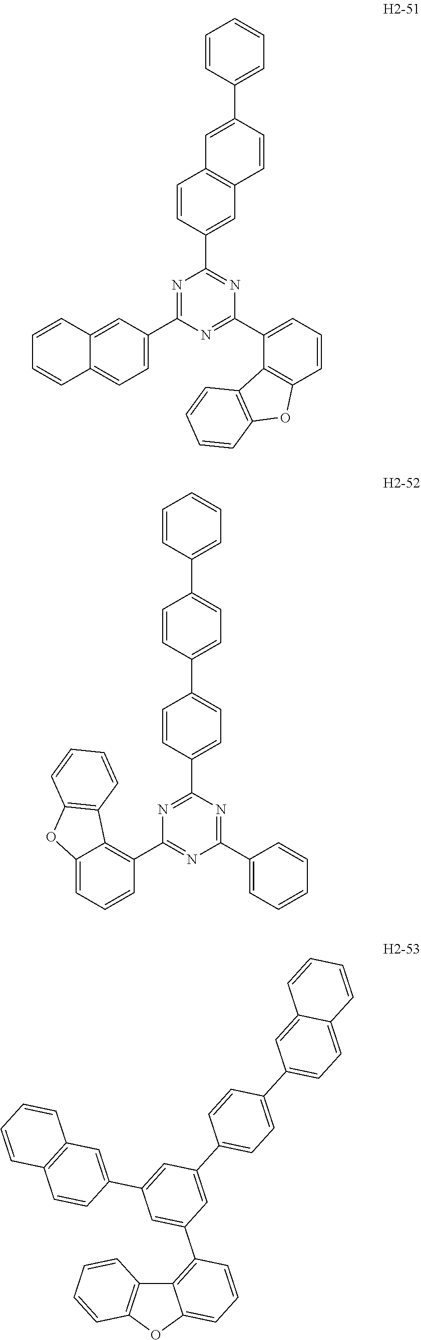 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccccc5)nc(-c5cccc6oc7ccccc7c56)n4)cc3)cc2)cc1 ; c1ccc2cc(-c3ccc(-c4cc(-c5ccc6ccccc6c5)cc(-c5cccc6oc7ccccc7c56)c4)cc3)ccc2c1]

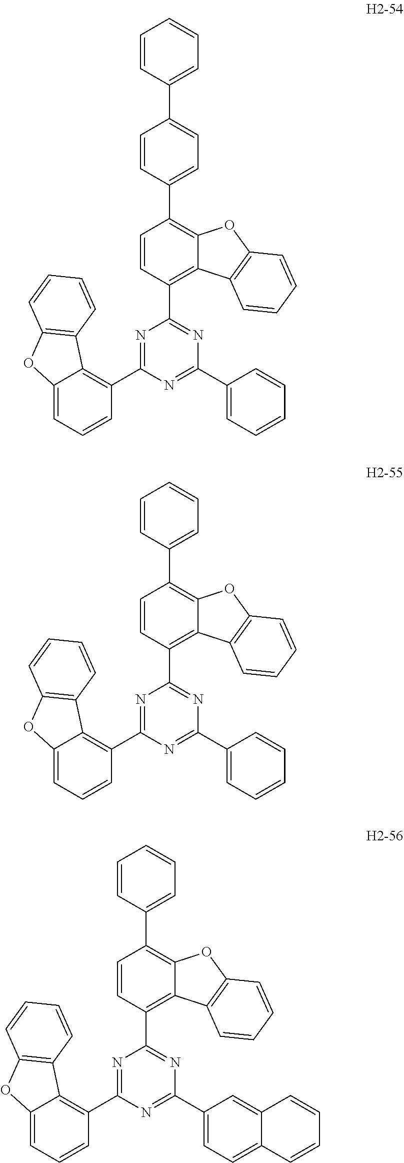 [SMILES: c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2nc(-c3cccc4oc5ccccc5c34)nc(-c3ccc(-c4ccccc4)c4oc5ccccc5c34)n2)cc1 ; c1ccc(-c2ccc(-c3nc(-c4ccc5ccccc5c4)nc(-c4cccc5oc6ccccc6c45)n3)c3c2oc2ccccc23)cc1]

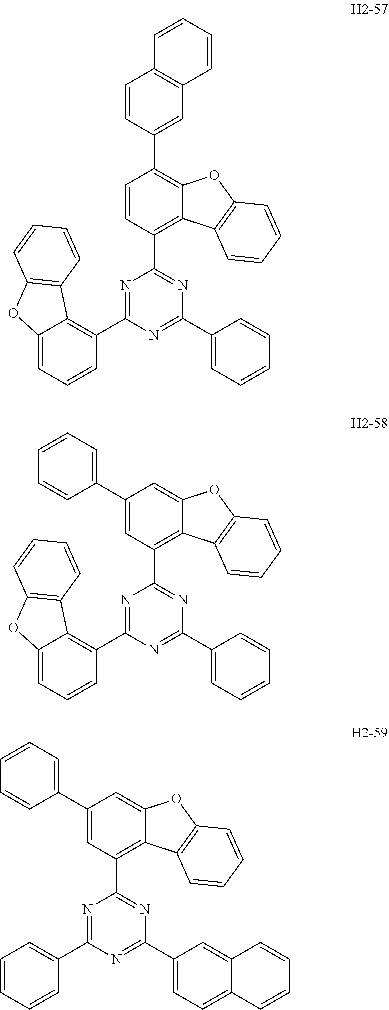 [SMILES: c1ccc(-c2nc(-c3cccc4oc5ccccc5c34)nc(-c3ccc(-c4ccc5ccccc5c4)c4oc5ccccc5c34)n2)cc1 ; c1ccc(-c2cc(-c3nc(-c4ccccc4)nc(-c4cccc5oc6ccccc6c45)n3)c3c(c2)oc2ccccc23)cc1 ; c1ccc(-c2cc(-c3nc(-c4ccccc4)nc(-c4ccc5ccccc5c4)n3)c3c(c2)oc2ccccc23)cc1]

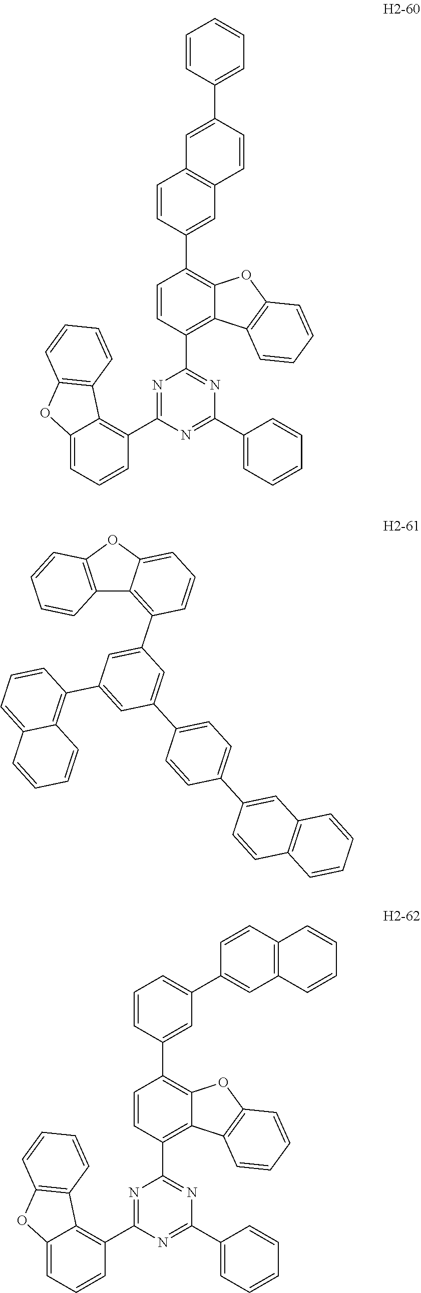 [SMILES: c1ccc(-c2ccc3cc(-c4ccc(-c5nc(-c6ccccc6)nc(-c6cccc7oc8ccccc8c67)n5)c5c4oc4ccccc45)ccc3c2)cc1 ; c1ccc2cc(-c3ccc(-c4cc(-c5cccc6ccccc56)cc(-c5cccc6oc7ccccc7c56)c4)cc3)ccc2c1 ; c1ccc(-c2nc(-c3cccc4oc5ccccc5c34)nc(-c3ccc(-c4cccc(-c5ccc6ccccc6c5)c4)c4oc5ccccc5c34)n2)cc1]

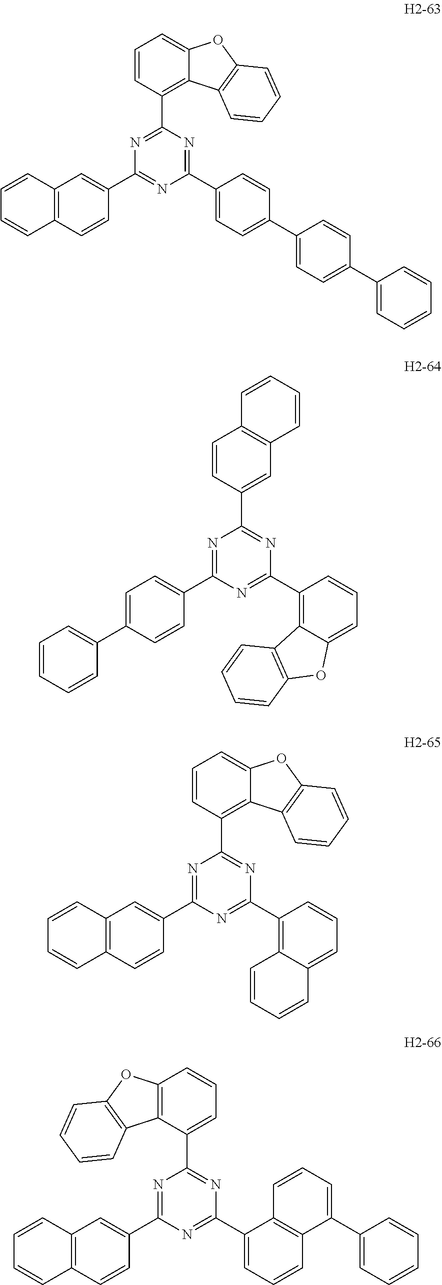 [SMILES: c1ccc(-c2ccc(-c3ccc(-c4nc(-c5ccc6ccccc6c5)nc(-c5cccc6oc7ccccc7c56)n4)cc3)cc2)cc1 ; c1ccc(-c2ccc(-c3nc(-c4ccc5ccccc5c4)nc(-c4cccc5oc6ccccc6c45)n3)cc2)cc1 ; c1ccc2cc(-c3nc(-c4cccc5ccccc45)nc(-c4cccc5oc6ccccc6c45)n3)ccc2c1 ; c1ccc(-c2cccc3c(-c4nc(-c5ccc6ccccc6c5)nc(-c5cccc6oc7ccccc7c56)n4)cccc23)cc1]

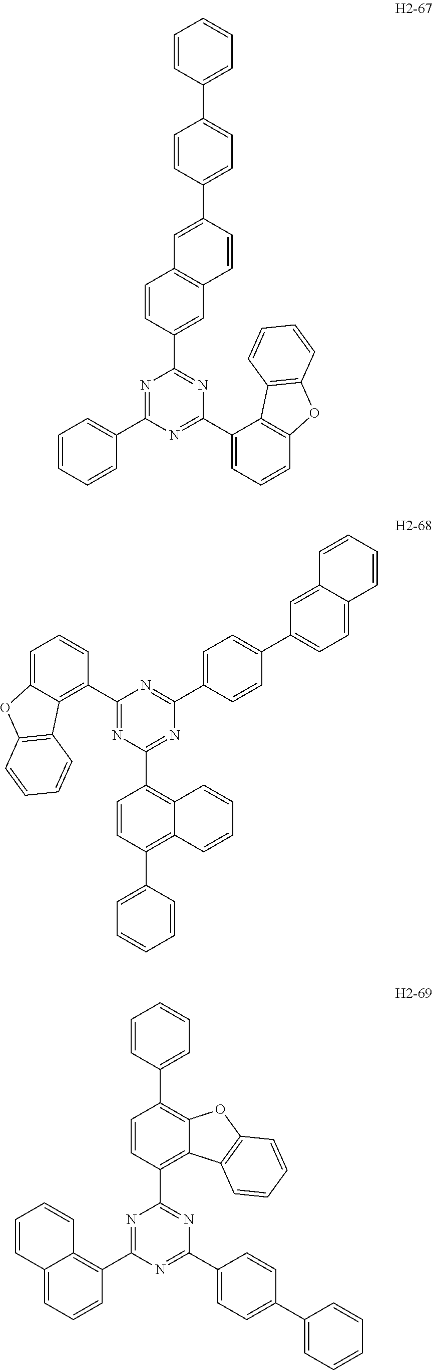 [SMILES: c1ccc(-c2ccc(-c3ccc4cc(-c5nc(-c6ccccc6)nc(-c6cccc7oc8ccccc8c67)n5)ccc4c3)cc2)cc1 ; c1ccc(-c2ccc(-c3nc(-c4ccc(-c5ccc6ccccc6c5)cc4)nc(-c4cccc5oc6ccccc6c45)n3)c3ccccc23)cc1 ; c1ccc(-c2ccc(-c3nc(-c4cccc5ccccc45)nc(-c4ccc(-c5ccccc5)c5oc6ccccc6c45)n3)cc2)cc1]

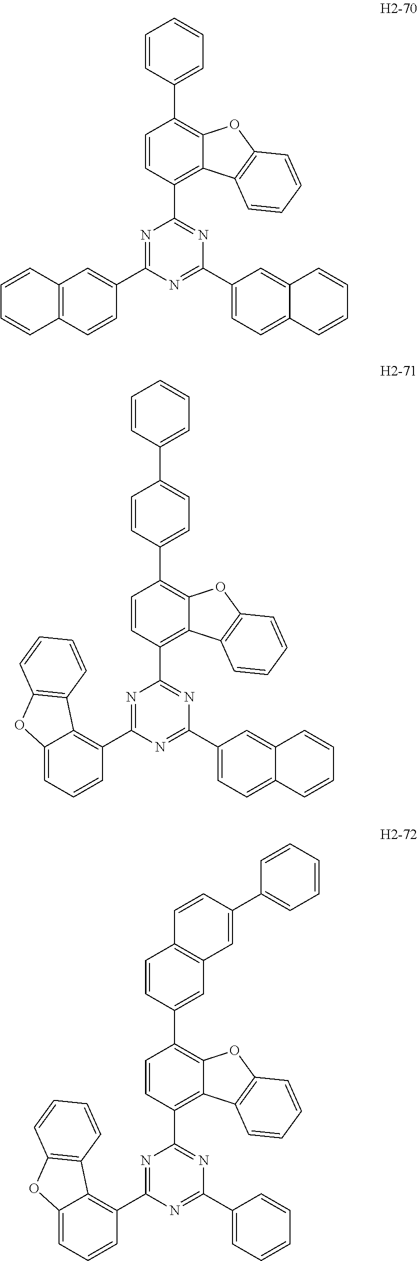 [SMILES: c1ccc(-c2ccc(-c3nc(-c4ccc5ccccc5c4)nc(-c4ccc5ccccc5c4)n3)c3c2oc2ccccc23)cc1 ; c1ccc(-c2ccc(-c3ccc(-c4nc(-c5ccc6ccccc6c5)nc(-c5cccc6oc7ccccc7c56)n4)c4c3oc3ccccc34)cc2)cc1 ; c1ccc(-c2ccc3ccc(-c4ccc(-c5nc(-c6ccccc6)nc(-c6cccc7oc8ccccc8c67)n5)c5c4oc4ccccc45)cc3c2)cc1]

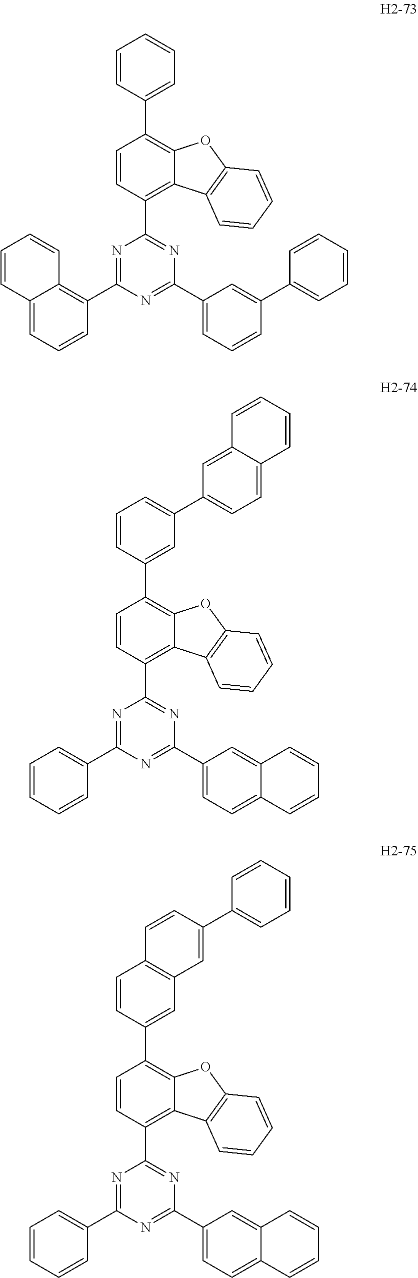 [SMILES: c1ccc(-c2cccc(-c3nc(-c4cccc5ccccc45)nc(-c4ccc(-c5ccccc5)c5oc6ccccc6c45)n3)c2)cc1 ; c1ccc(-c2nc(-c3ccc4ccccc4c3)nc(-c3ccc(-c4cccc(-c5ccc6ccccc6c5)c4)c4oc5ccccc5c34)n2)cc1 ; c1ccc(-c2ccc3ccc(-c4ccc(-c5nc(-c6ccccc6)nc(-c6ccc7ccccc7c6)n5)c5c4oc4ccccc45)cc3c2)cc1]

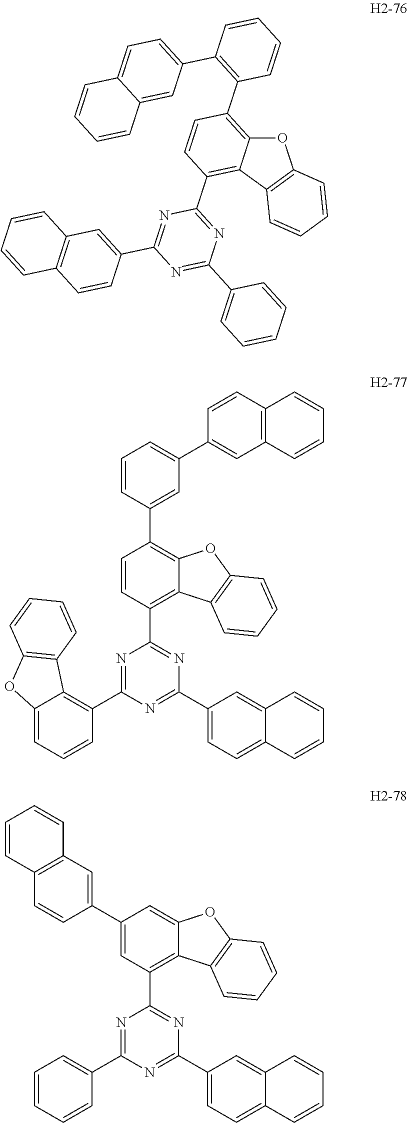

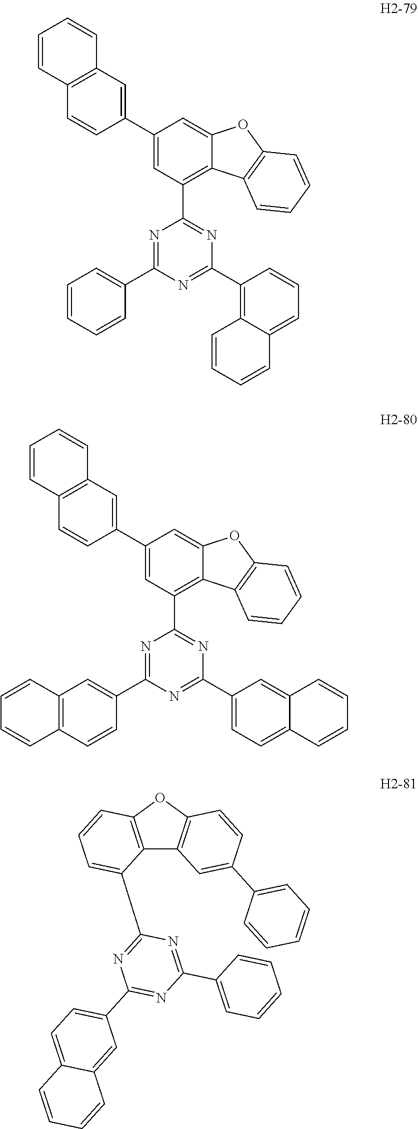 [SMILES: c1ccc(-c2nc(-c3cccc4ccccc34)nc(-c3cc(-c4ccc5ccccc5c4)cc4oc5ccccc5c34)n2)cc1 ; c1ccc2cc(-c3cc(-c4nc(-c5ccc6ccccc6c5)nc(-c5ccc6ccccc6c5)n4)c4c(c3)oc3ccccc34)ccc2c1 ; c1ccc(-c2ccc3oc4cccc(-c5nc(-c6ccccc6)nc(-c6ccc7ccccc7c6)n5)c4c3c2)cc1]

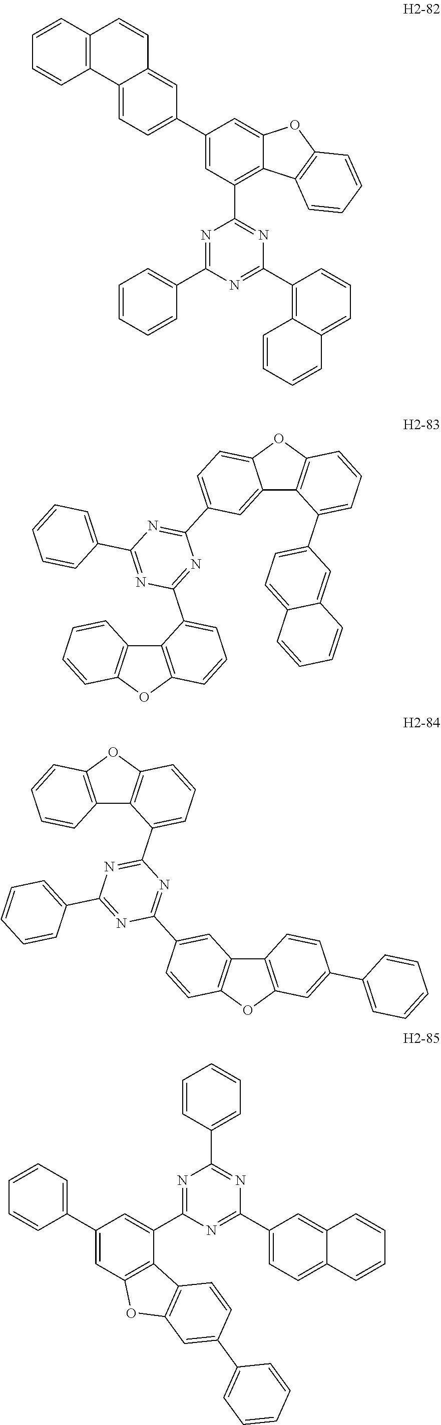 [SMILES: c1ccc(-c2nc(-c3cccc4ccccc34)nc(-c3cc(-c4ccc5c(ccc6ccccc65)c4)cc4oc5ccccc5c34)n2)cc1 ; c1ccc(-c2nc(-c3ccc4oc5cccc(-c6ccc7ccccc7c6)c5c4c3)nc(-c3cccc4oc5ccccc5c34)n2)cc1 ; c1ccc(-c2ccc3c(c2)oc2ccc(-c4nc(-c5ccccc5)nc(-c5cccc6oc7ccccc7c56)n4)cc23)cc1 ; c1ccc(-c2ccc3c(c2)oc2cc(-c4ccccc4)cc(-c4nc(-c5ccccc5)nc(-c5ccc6ccccc6c5)n4)c23)cc1]

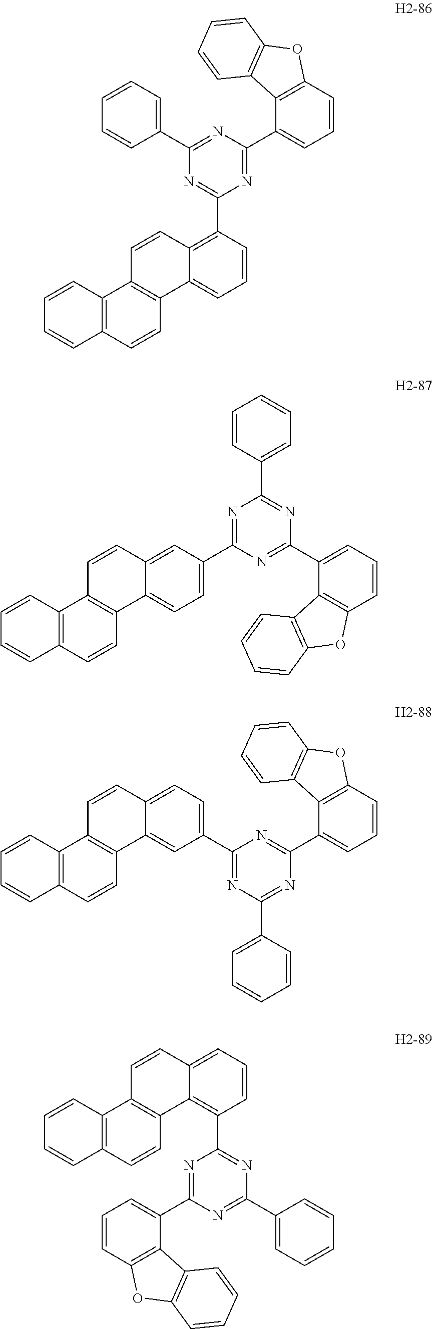 [SMILES: c1ccc(-c2nc(-c3cccc4c3ccc3c5ccccc5ccc43)nc(-c3cccc4oc5ccccc5c34)n2)cc1 ; c1ccc(-c2nc(-c3ccc4c(ccc5c6ccccc6ccc45)c3)nc(-c3cccc4oc5ccccc5c34)n2)cc1 ; c1ccc(-c2nc(-c3ccc4ccc5c6ccccc6ccc5c4c3)nc(-c3cccc4oc5ccccc5c34)n2)cc1 ; c1ccc(-c2nc(-c3cccc4ccc5c6ccccc6ccc5c34)nc(-c3cccc4oc5ccccc5c34)n2)cc1]

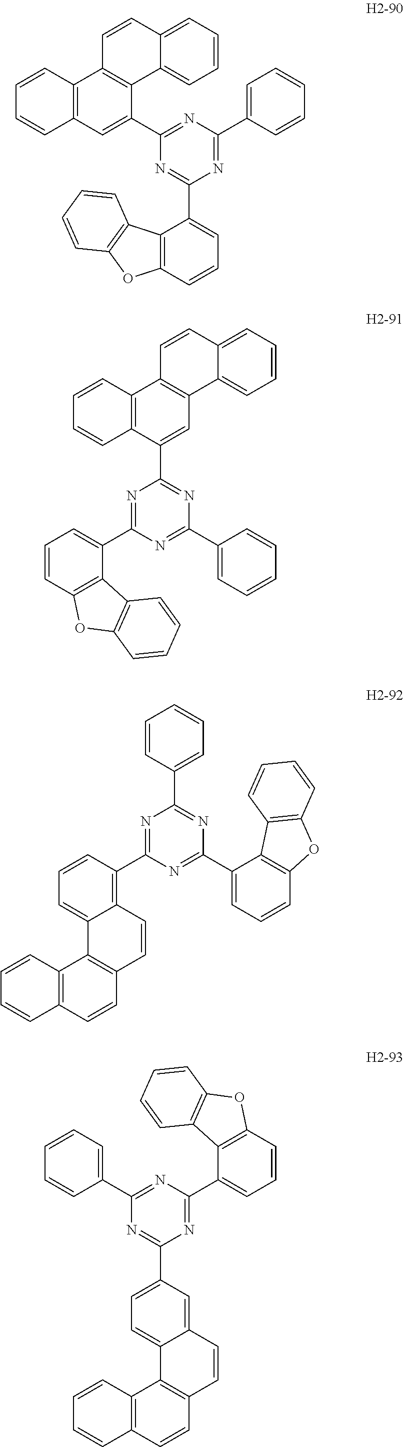

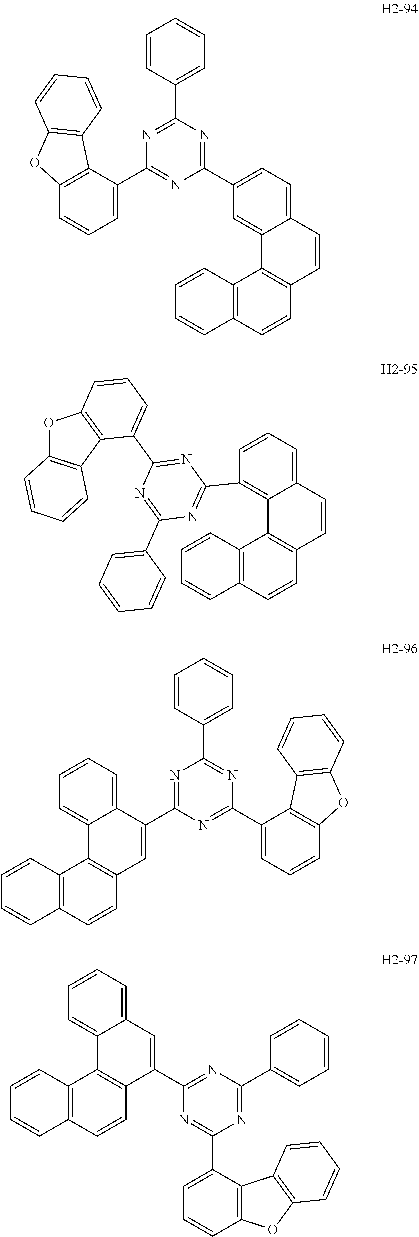

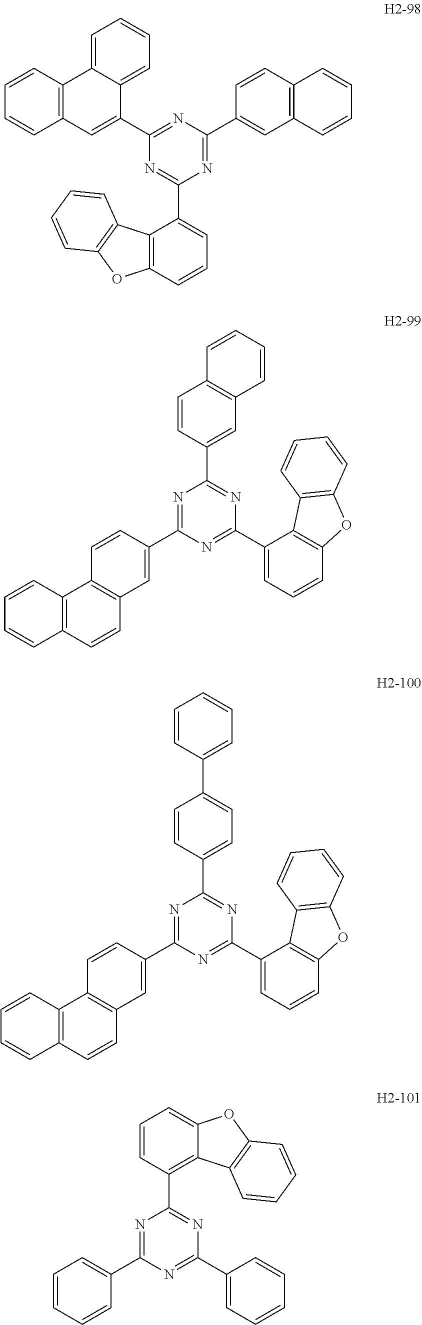 [SMILES: c1ccc2cc(-c3nc(-c4cc5ccccc5c5ccccc45)nc(-c4cccc5oc6ccccc6c45)n3)ccc2c1 ; c1ccc2cc(-c3nc(-c4ccc5c(ccc6ccccc65)c4)nc(-c4cccc5oc6ccccc6c45)n3)ccc2c1 ; c1ccc(-c2ccc(-c3nc(-c4ccc5c(ccc6ccccc65)c4)nc(-c4cccc5oc6ccccc6c45)n3)cc2)cc1 ; c1ccc(-c2nc(-c3ccccc3)nc(-c3cccc4oc5ccccc5c34)n2)cc1]

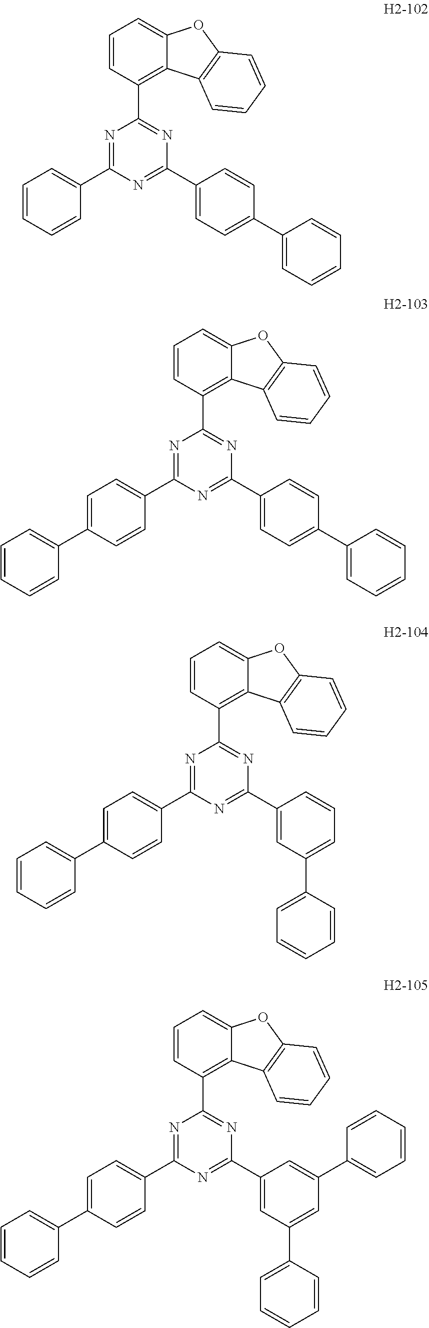 [SMILES: c1ccc(-c2ccc(-c3nc(-c4ccccc4)nc(-c4cccc5oc6ccccc6c45)n3)cc2)cc1 ; c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4cccc(-c5ccccc5)c4)nc(-c4cccc5oc6ccccc6c45)n3)cc2)cc1 ; c1ccc(-c2ccc(-c3nc(-c4cc(-c5ccccc5)cc(-c5ccccc5)c4)nc(-c4cccc5oc6ccccc6c45)n3)cc2)cc1]

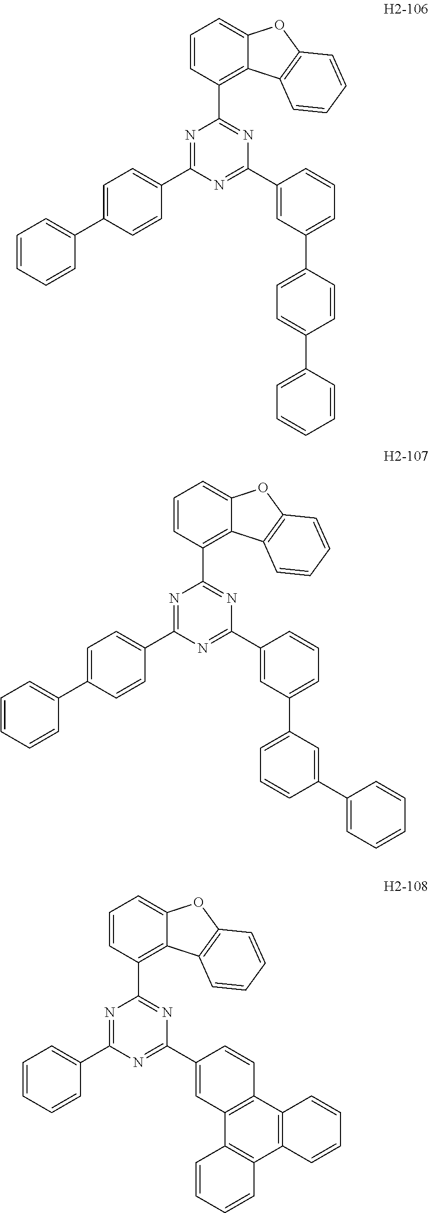 [SMILES: c1ccc(-c2ccc(-c3cccc(-c4nc(-c5ccc(-c6ccccc6)cc5)nc(-c5cccc6oc7ccccc7c56)n4)c3)cc2)cc1 ; c1ccc(-c2ccc(-c3nc(-c4cccc(-c5cccc(-c6ccccc6)c5)c4)nc(-c4cccc5oc6ccccc6c45)n3)cc2)cc1 ; c1ccc(-c2nc(-c3ccc4c5ccccc5c5ccccc5c4c3)nc(-c3cccc4oc5ccccc5c34)n2)cc1]

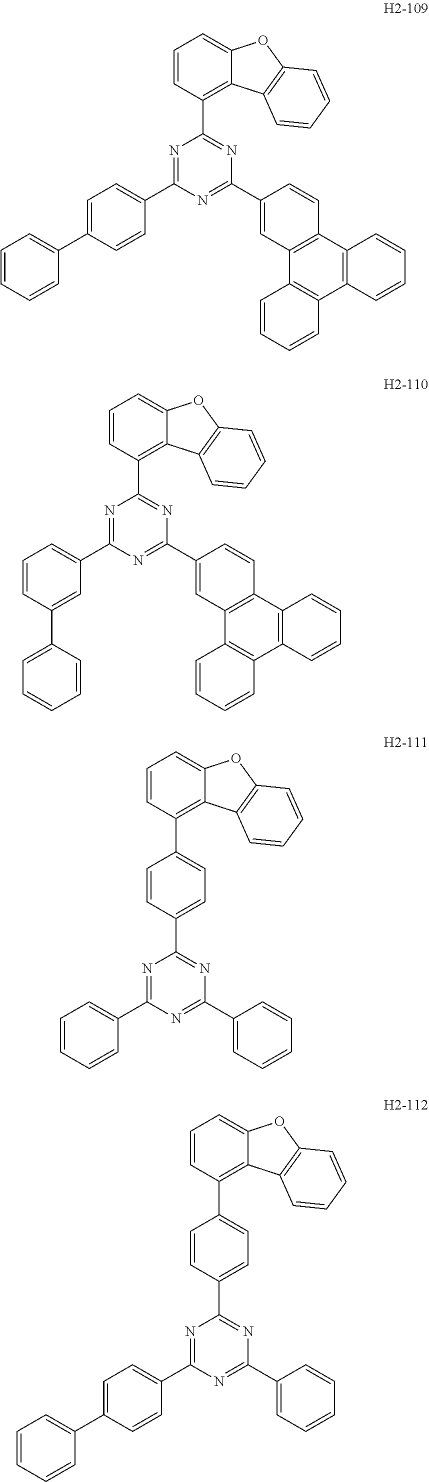 [SMILES: c1ccc(-c2ccc(-c3nc(-c4ccc5c6ccccc6c6ccccc6c5c4)nc(-c4cccc5oc6ccccc6c45)n3)cc2)cc1 ; c1ccc(-c2cccc(-c3nc(-c4ccc5c6ccccc6c6ccccc6c5c4)nc(-c4cccc5oc6ccccc6c45)n3)c2)cc1 ; c1ccc(-c2nc(-c3ccccc3)nc(-c3ccc(-c4cccc5oc6ccccc6c45)cc3)n2)cc1 ; c1ccc(-c2ccc(-c3nc(-c4ccccc4)nc(-c4ccc(-c5cccc6oc7ccccc7c56)cc4)n3)cc2)cc1]

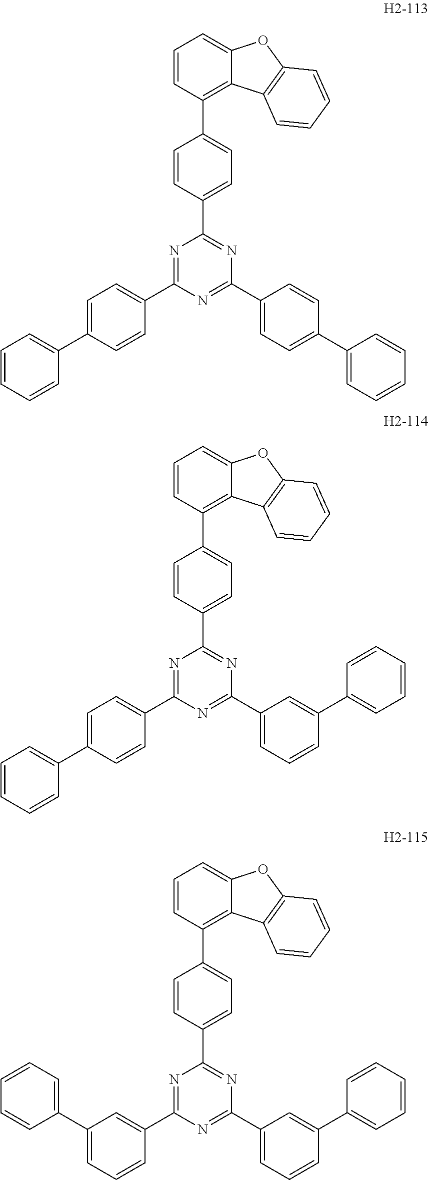 [SMILES: c1ccc(-c2ccc(-c3nc(-c4ccc(-c5ccccc5)cc4)nc(-c4ccc(-c5cccc6oc7ccccc7c56)cc4)n3)cc2)cc1 ; c1ccc(-c2ccc(-c3nc(-c4ccc(-c5cccc6oc7ccccc7c56)cc4)nc(-c4cccc(-c5ccccc5)c4)n3)cc2)cc1 ; c1ccc(-c2cccc(-c3nc(-c4ccc(-c5cccc6oc7ccccc7c56)cc4)nc(-c4cccc(-c5ccccc5)c4)n3)c2)cc1]

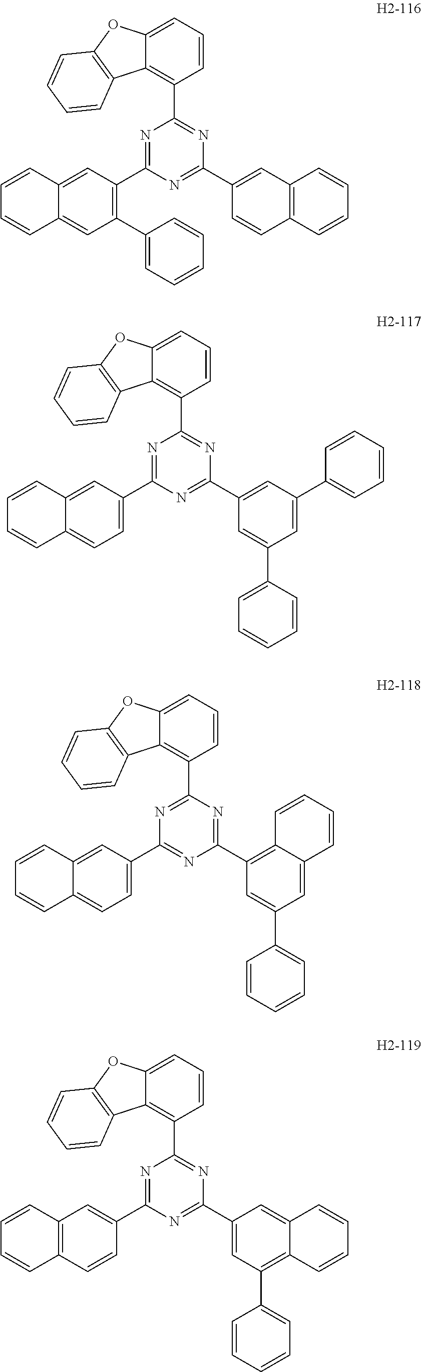 [SMILES: c1ccc(-c2cc3ccccc3cc2-c2nc(-c3ccc4ccccc4c3)nc(-c3cccc4oc5ccccc5c34)n2)cc1 ; c1ccc(-c2cc(-c3ccccc3)cc(-c3nc(-c4ccc5ccccc5c4)nc(-c4cccc5oc6ccccc6c45)n3)c2)cc1 ; c1ccc(-c2cc(-c3nc(-c4ccc5ccccc5c4)nc(-c4cccc5oc6ccccc6c45)n3)c3ccccc3c2)cc1 ; c1ccc(-c2cc(-c3nc(-c4ccc5ccccc5c4)nc(-c4cccc5oc6ccccc6c45)n3)cc3ccccc23)cc1]

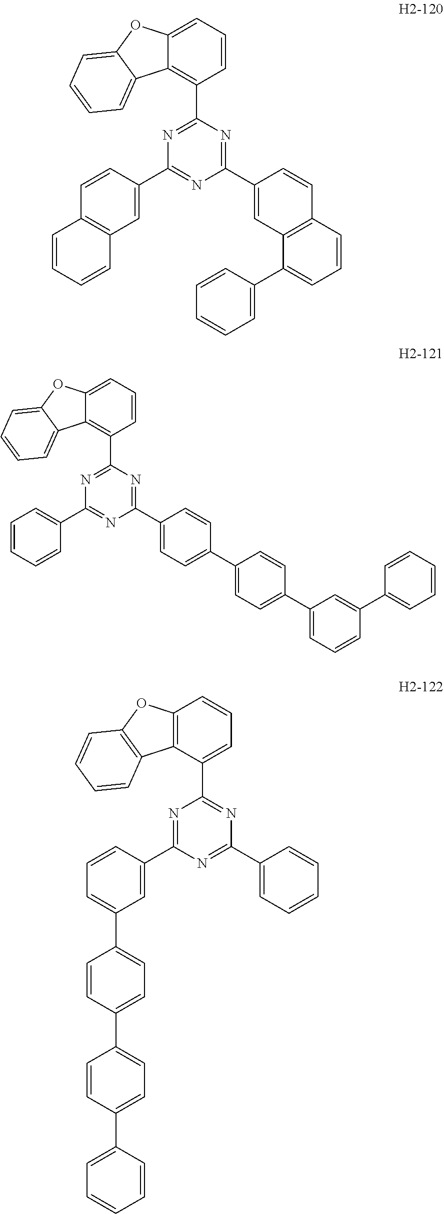 [SMILES: c1ccc(-c2cccc3ccc(-c4nc(-c5ccc6ccccc6c5)nc(-c5cccc6oc7ccccc7c56)n4)cc23)cc1 ; c1ccc(-c2cccc(-c3ccc(-c4ccc(-c5nc(-c6ccccc6)nc(-c6cccc7oc8ccccc8c67)n5)cc4)cc3)c2)cc1 ; c1ccc(-c2ccc(-c3ccc(-c4cccc(-c5nc(-c6ccccc6)nc(-c6cccc7oc8ccccc8c67)n5)c4)cc3)cc2)cc1]

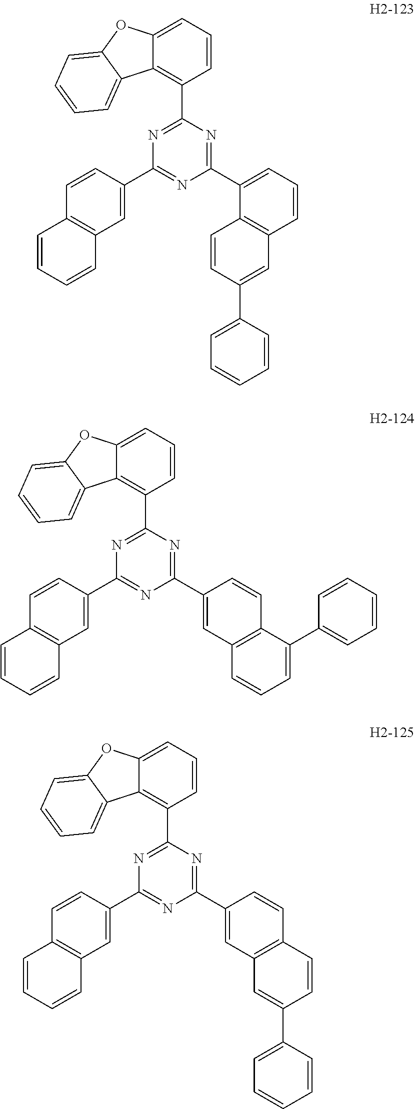 [SMILES: c1ccc(-c2ccc3c(-c4nc(-c5ccc6ccccc6c5)nc(-c5cccc6oc7ccccc7c56)n4)cccc3c2)cc1 ; c1ccc(-c2cccc3cc(-c4nc(-c5ccc6ccccc6c5)nc(-c5cccc6oc7ccccc7c56)n4)ccc23)cc1 ; c1ccc(-c2ccc3ccc(-c4nc(-c5ccc6ccccc6c5)nc(-c5cccc6oc7ccccc7c56)n4)cc3c2)cc1]

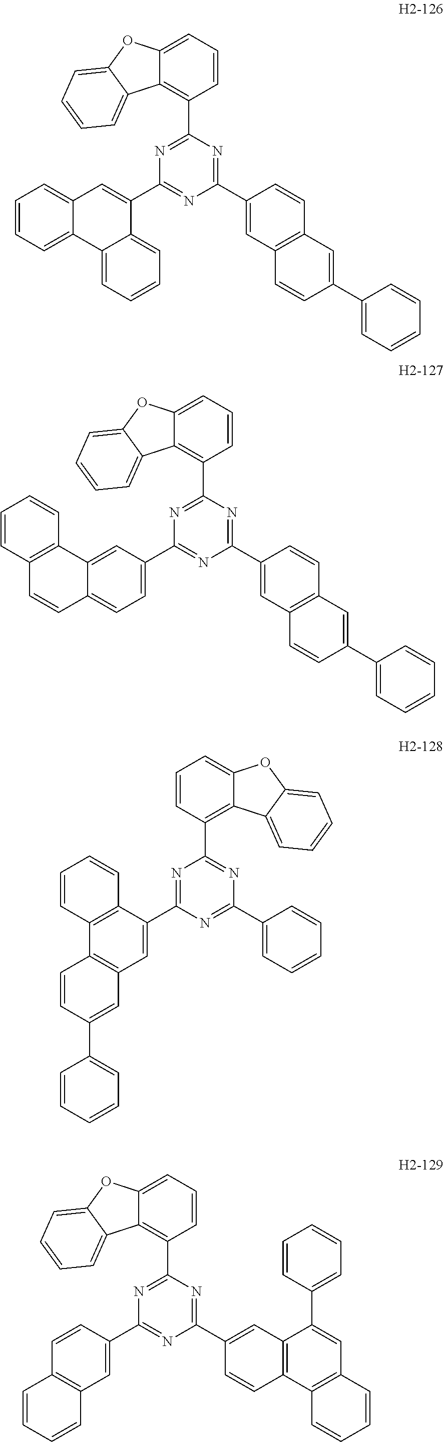 [SMILES: c1ccc(-c2ccc3cc(-c4nc(-c5cc6ccccc6c6ccccc56)nc(-c5cccc6oc7ccccc7c56)n4)ccc3c2)cc1 ; c1ccc(-c2ccc3cc(-c4nc(-c5ccc6ccc7ccccc7c6c5)nc(-c5cccc6oc7ccccc7c56)n4)ccc3c2)cc1 ; c1ccc(-c2ccc3c(c2)cc(-c2nc(-c4ccccc4)nc(-c4cccc5oc6ccccc6c45)n2)c2ccccc23)cc1 ; c1ccc(-c2cc3ccccc3c3ccc(-c4nc(-c5ccc6ccccc6c5)nc(-c5cccc6oc7ccccc7c56)n4)cc23)cc1]

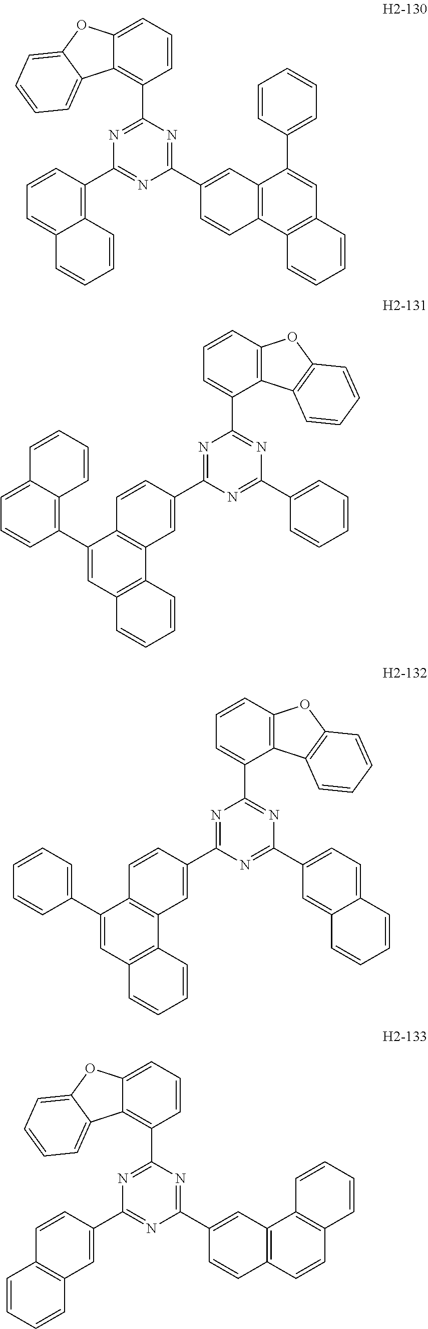 [SMILES: c1ccc(-c2cc3ccccc3c3ccc(-c4nc(-c5cccc6ccccc56)nc(-c5cccc6oc7ccccc7c56)n4)cc23)cc1 ; c1ccc(-c2nc(-c3ccc4c(-c5cccc6ccccc56)cc5ccccc5c4c3)nc(-c3cccc4oc5ccccc5c34)n2)cc1 ; c1ccc(-c2cc3ccccc3c3cc(-c4nc(-c5ccc6ccccc6c5)nc(-c5cccc6oc7ccccc7c56)n4)ccc23)cc1 ; c1ccc2cc(-c3nc(-c4ccc5ccc6ccccc6c5c4)nc(-c4cccc5oc6ccccc6c45)n3)ccc2c1]

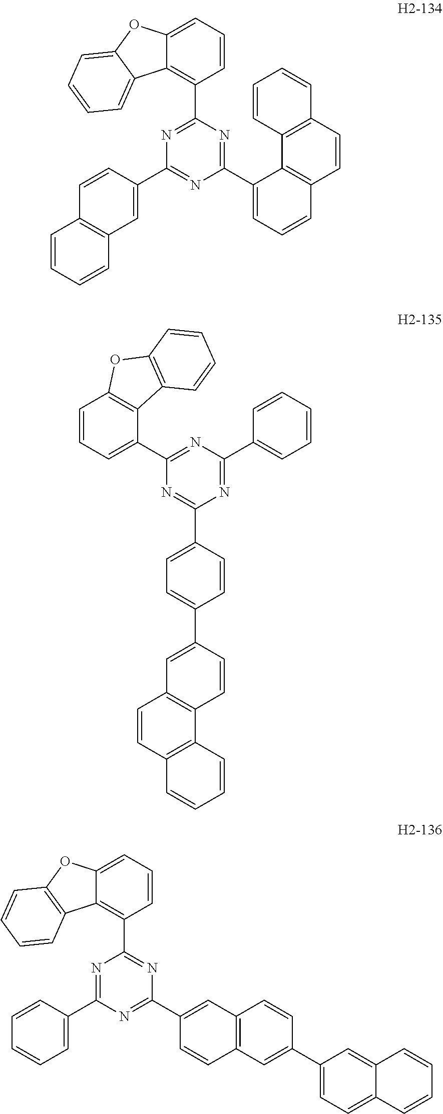 [SMILES: c1ccc2cc(-c3nc(-c4cccc5ccc6ccccc6c45)nc(-c4cccc5oc6ccccc6c45)n3)ccc2c1 ; c1ccc(-c2nc(-c3ccc(-c4ccc5c(ccc6ccccc65)c4)cc3)nc(-c3cccc4oc5ccccc5c34)n2)cc1 ; c1ccc(-c2nc(-c3ccc4cc(-c5ccc6ccccc6c5)ccc4c3)nc(-c3cccc4oc5ccccc5c34)n2)cc1]

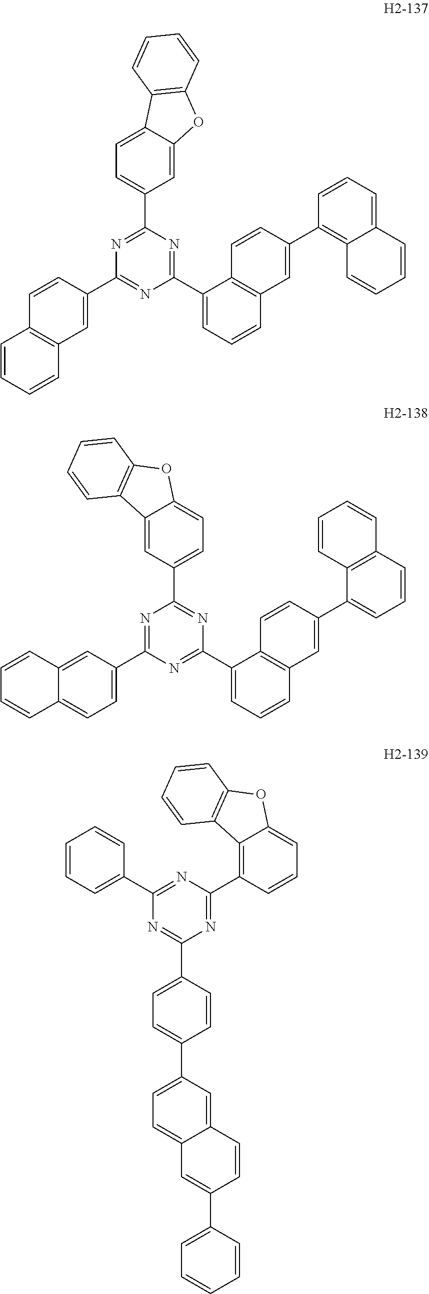 [SMILES: c1ccc2cc(-c3nc(-c4ccc5c(c4)oc4ccccc45)nc(-c4cccc5cc(-c6cccc7ccccc67)ccc45)n3)ccc2c1 ; c1ccc2cc(-c3nc(-c4ccc5oc6ccccc6c5c4)nc(-c4cccc5cc(-c6cccc7ccccc67)ccc45)n3)ccc2c1 ; c1ccc(-c2ccc3cc(-c4ccc(-c5nc(-c6ccccc6)nc(-c6cccc7oc8ccccc8c67)n5)cc4)ccc3c2)cc1]

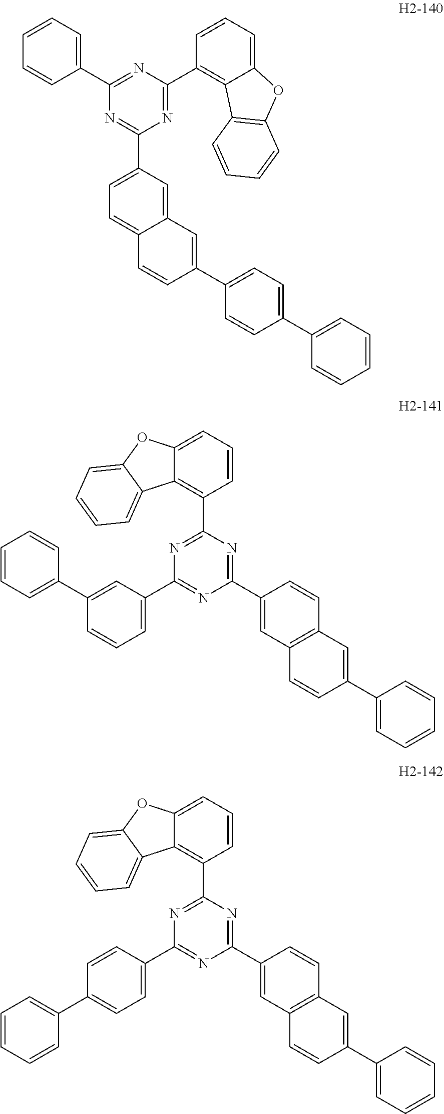 [SMILES: c1ccc(-c2ccc(-c3ccc4ccc(-c5nc(-c6ccccc6)nc(-c6cccc7oc8ccccc8c67)n5)cc4c3)cc2)cc1 ; c1ccc(-c2cccc(-c3nc(-c4ccc5cc(-c6ccccc6)ccc5c4)nc(-c4cccc5oc6ccccc6c45)n3)c2)cc1 ; c1ccc(-c2ccc(-c3nc(-c4ccc5cc(-c6ccccc6)ccc5c4)nc(-c4cccc5oc6ccccc6c45)n3)cc2)cc1]

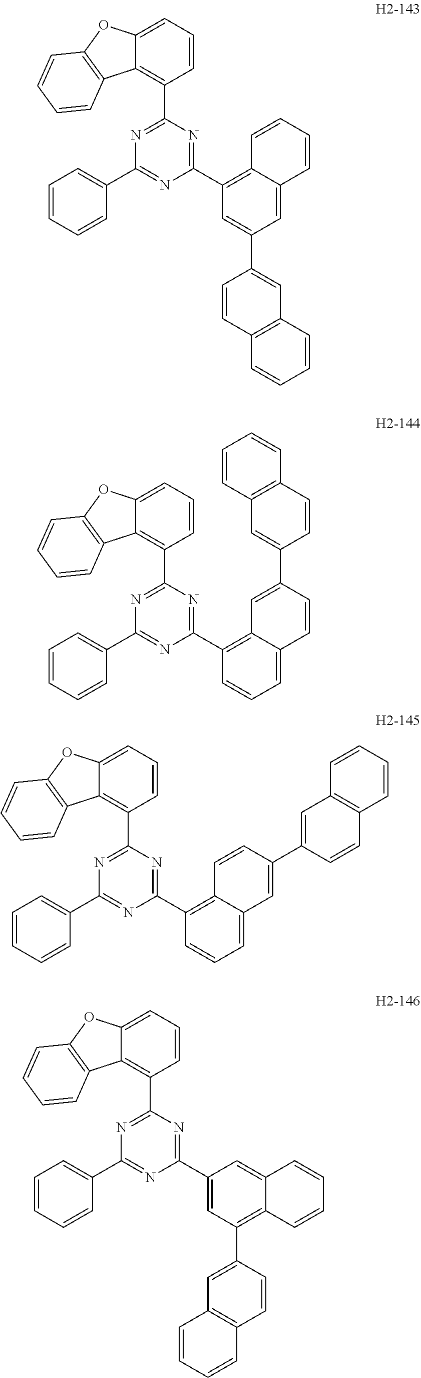 [SMILES: c1ccc(-c2nc(-c3cc(-c4ccc5ccccc5c4)cc4ccccc34)nc(-c3cccc4oc5ccccc5c34)n2)cc1 ; c1ccc(-c2nc(-c3cccc4ccc(-c5ccc6ccccc6c5)cc34)nc(-c3cccc4oc5ccccc5c34)n2)cc1 ; c1ccc(-c2nc(-c3cccc4cc(-c5ccc6ccccc6c5)ccc34)nc(-c3cccc4oc5ccccc5c34)n2)cc1 ; c1ccc(-c2nc(-c3cc(-c4ccc5ccccc5c4)c4ccccc4c3)nc(-c3cccc4oc5ccccc5c34)n2)cc1]

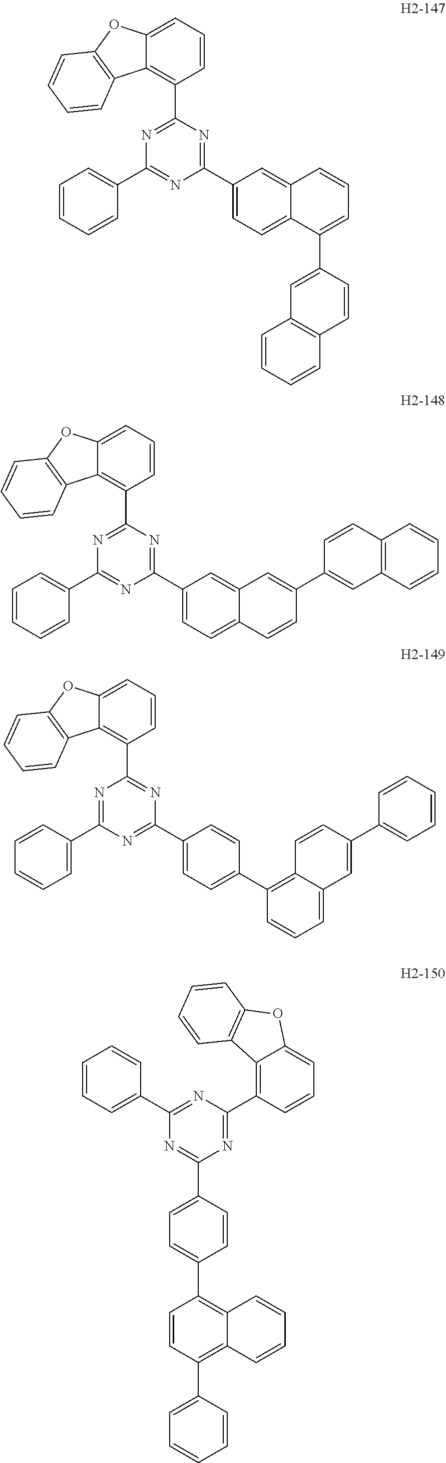 [SMILES: c1ccc(-c2nc(-c3ccc4c(-c5ccc6ccccc6c5)cccc4c3)nc(-c3cccc4oc5ccccc5c34)n2)cc1 ; c1ccc(-c2nc(-c3ccc4ccc(-c5ccc6ccccc6c5)cc4c3)nc(-c3cccc4oc5ccccc5c34)n2)cc1 ; c1ccc(-c2ccc3c(-c4ccc(-c5nc(-c6ccccc6)nc(-c6cccc7oc8ccccc8c67)n5)cc4)cccc3c2)cc1 ; c1ccc(-c2nc(-c3ccc(-c4ccc(-c5ccccc5)c5ccccc45)cc3)nc(-c3cccc4oc5ccccc5c34)n2)cc1]

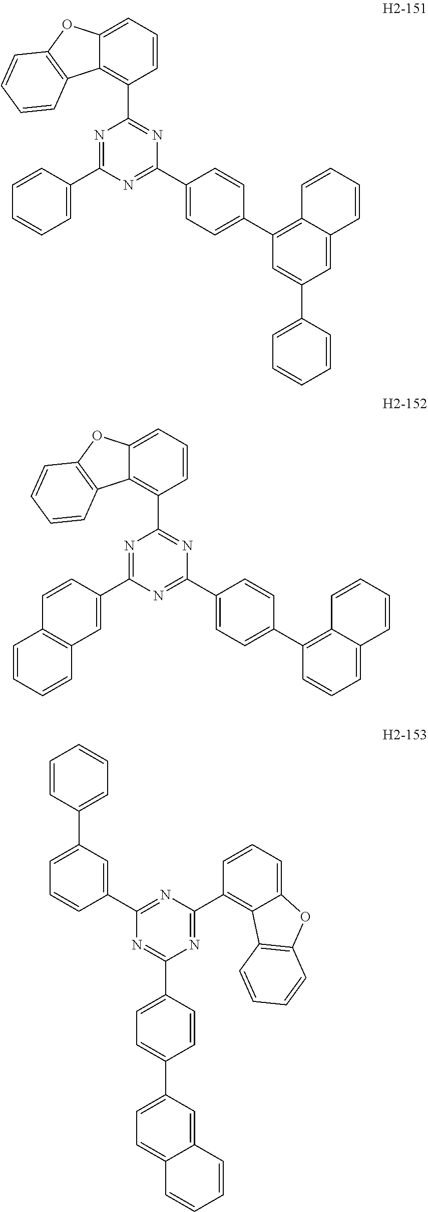 [SMILES: c1ccc(-c2cc(-c3ccc(-c4nc(-c5ccccc5)nc(-c5cccc6oc7ccccc7c56)n4)cc3)c3ccccc3c2)cc1 ; c1ccc2cc(-c3nc(-c4ccc(-c5cccc6ccccc56)cc4)nc(-c4cccc5oc6ccccc6c45)n3)ccc2c1 ; c1ccc(-c2cccc(-c3nc(-c4ccc(-c5ccc6ccccc6c5)cc4)nc(-c4cccc5oc6ccccc6c45)n3)c2)cc1]

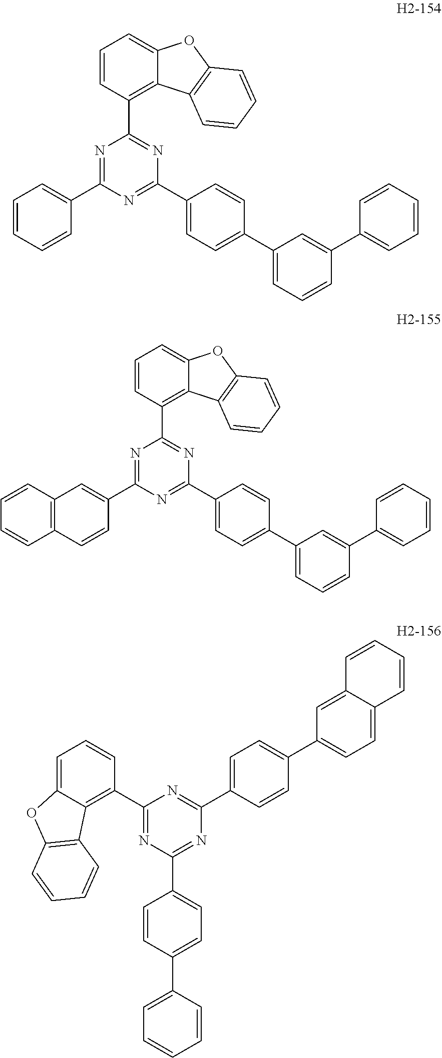 [SMILES: c1ccc(-c2cccc(-c3ccc(-c4nc(-c5ccccc5)nc(-c5cccc6oc7ccccc7c56)n4)cc3)c2)cc1 ; c1ccc(-c2cccc(-c3ccc(-c4nc(-c5ccc6ccccc6c5)nc(-c5cccc6oc7ccccc7c56)n4)cc3)c2)cc1 ; c1ccc(-c2ccc(-c3nc(-c4ccc(-c5ccc6ccccc6c5)cc4)nc(-c4cccc5oc6ccccc6c45)n3)cc2)cc1]

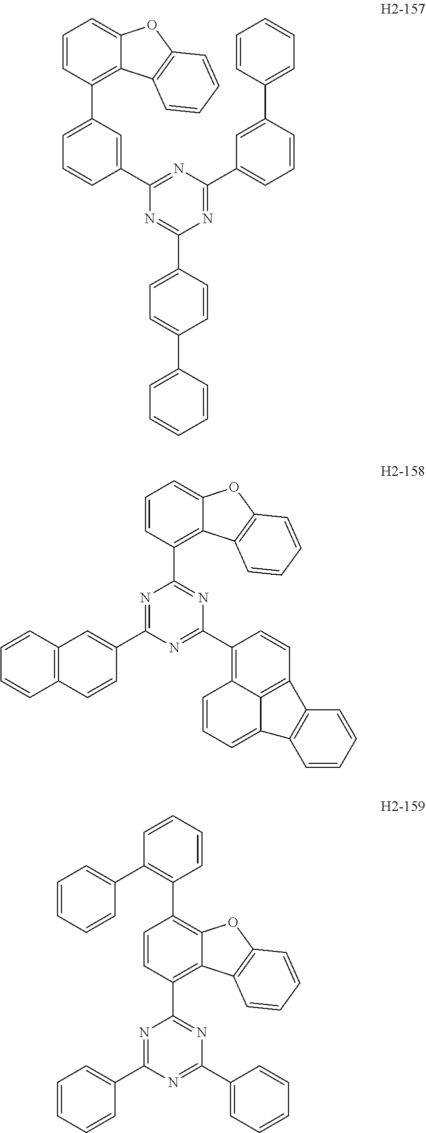

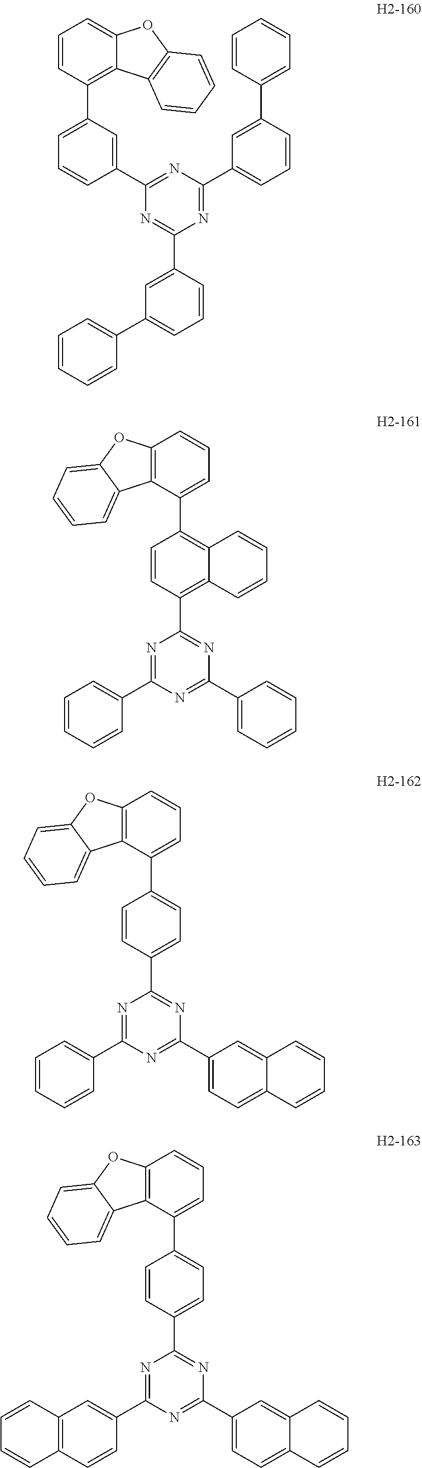 [SMILES: c1ccc(-c2cccc(-c3nc(-c4cccc(-c5ccccc5)c4)nc(-c4cccc(-c5cccc6oc7ccccc7c56)c4)n3)c2)cc1 ; c1ccc(-c2nc(-c3ccccc3)nc(-c3ccc(-c4cccc5oc6ccccc6c45)c4ccccc34)n2)cc1 ; c1ccc(-c2nc(-c3ccc(-c4cccc5oc6ccccc6c45)cc3)nc(-c3ccc4ccccc4c3)n2)cc1 ; c1ccc2cc(-c3nc(-c4ccc(-c5cccc6oc7ccccc7c56)cc4)nc(-c4ccc5ccccc5c4)n3)ccc2c1]

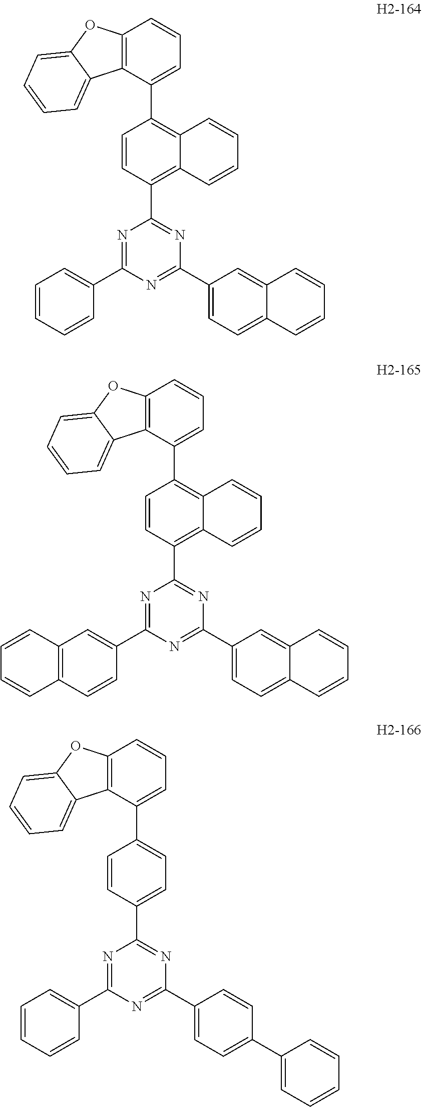 [SMILES: c1ccc(-c2nc(-c3ccc4ccccc4c3)nc(-c3ccc(-c4cccc5oc6ccccc6c45)c4ccccc34)n2)cc1 ; c1ccc2cc(-c3nc(-c4ccc5ccccc5c4)nc(-c4ccc(-c5cccc6oc7ccccc7c56)c5ccccc45)n3)ccc2c1 ; c1ccc(-c2ccc(-c3nc(-c4ccccc4)nc(-c4ccc(-c5cccc6oc7ccccc7c56)cc4)n3)cc2)cc1]

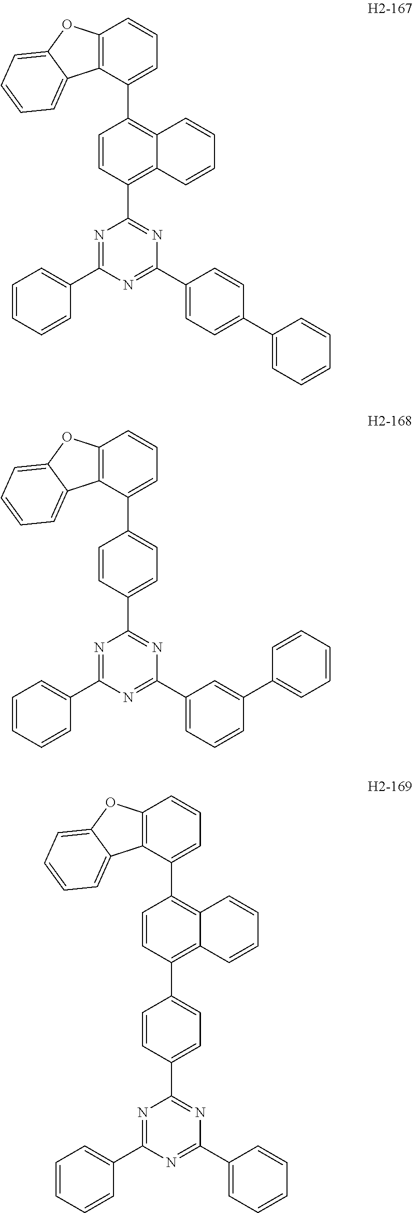 [SMILES: c1ccc(-c2ccc(-c3nc(-c4ccccc4)nc(-c4ccc(-c5cccc6oc7ccccc7c56)c5ccccc45)n3)cc2)cc1 ; c1ccc(-c2cccc(-c3nc(-c4ccccc4)nc(-c4ccc(-c5cccc6oc7ccccc7c56)cc4)n3)c2)cc1 ; c1ccc(-c2nc(-c3ccccc3)nc(-c3ccc(-c4ccc(-c5cccc6oc7ccccc7c56)c5ccccc45)cc3)n2)cc1]

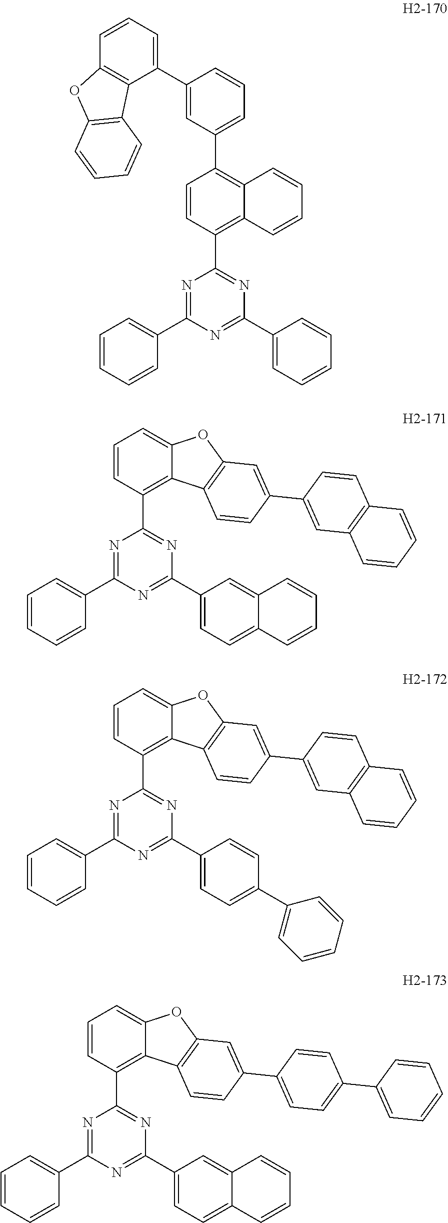

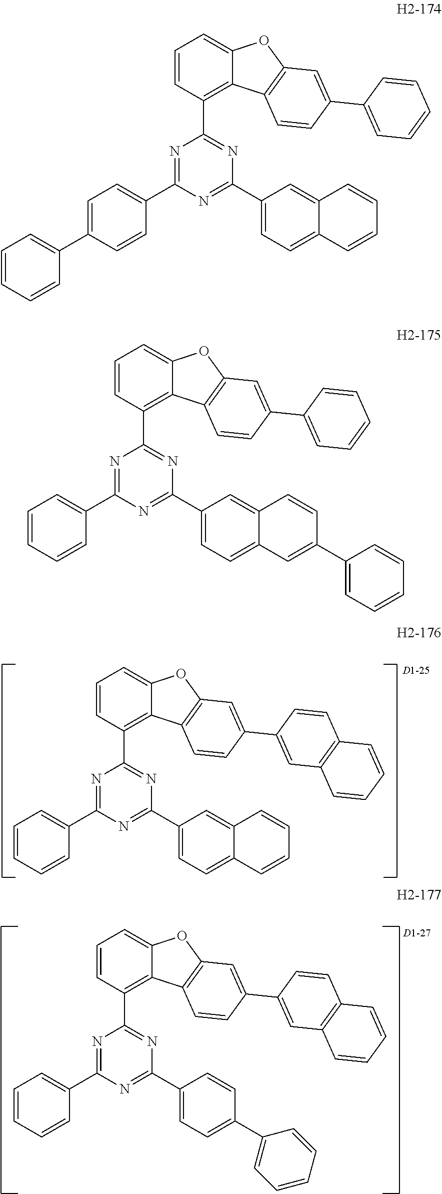

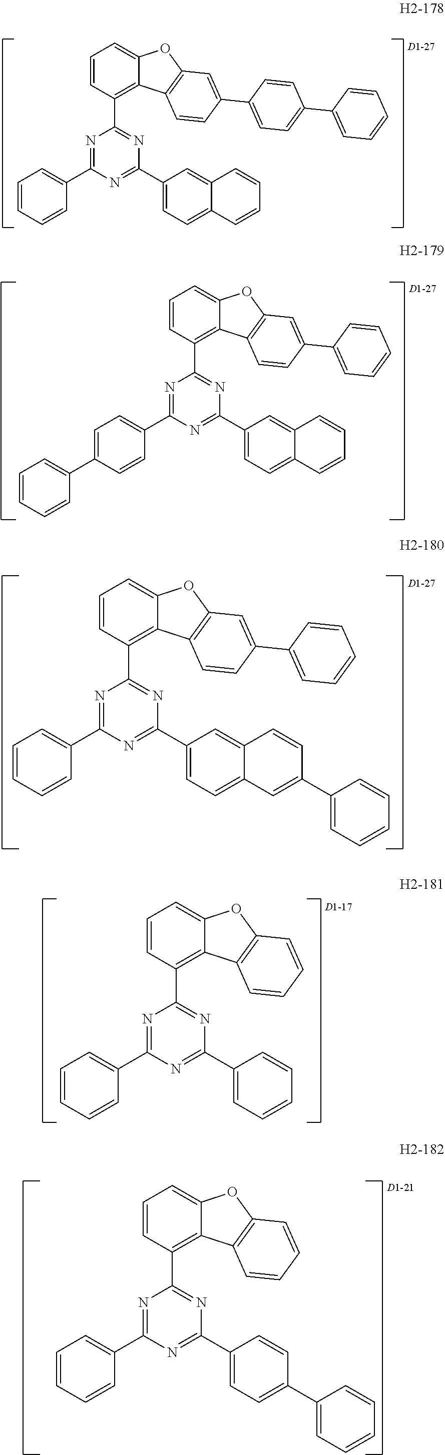

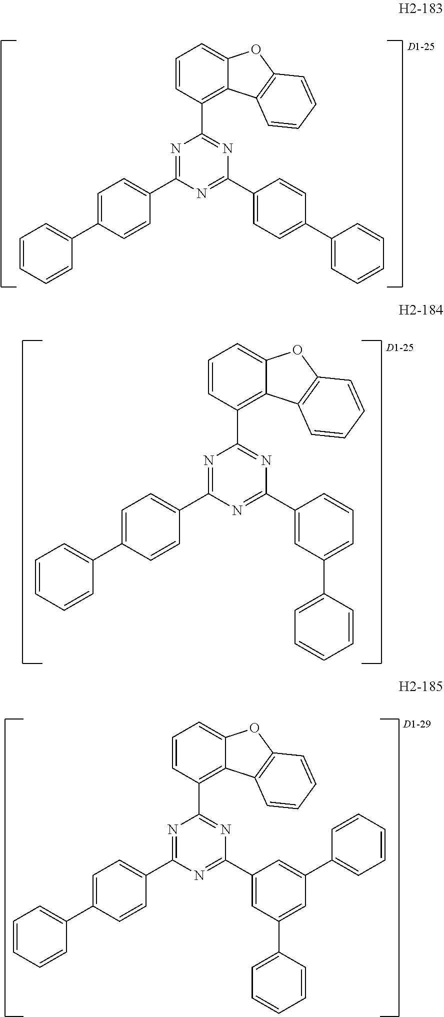

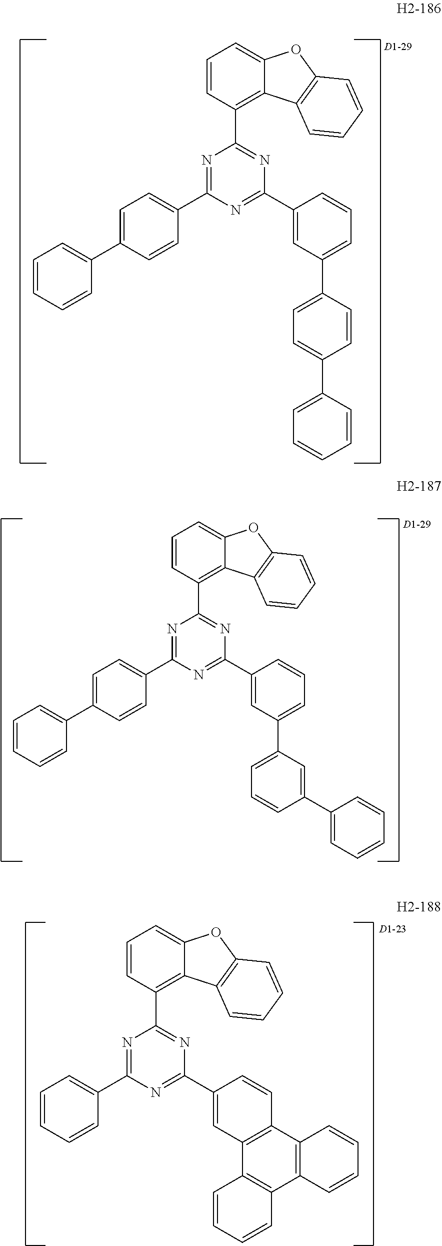

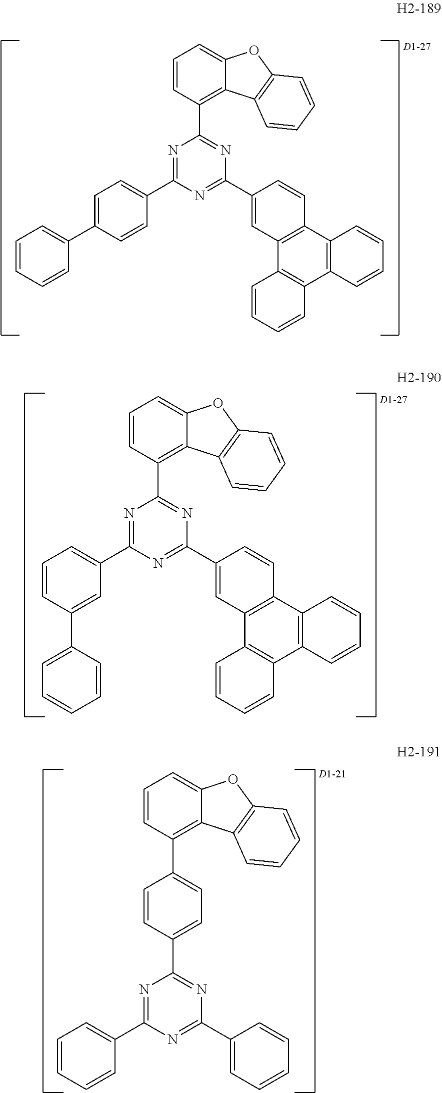

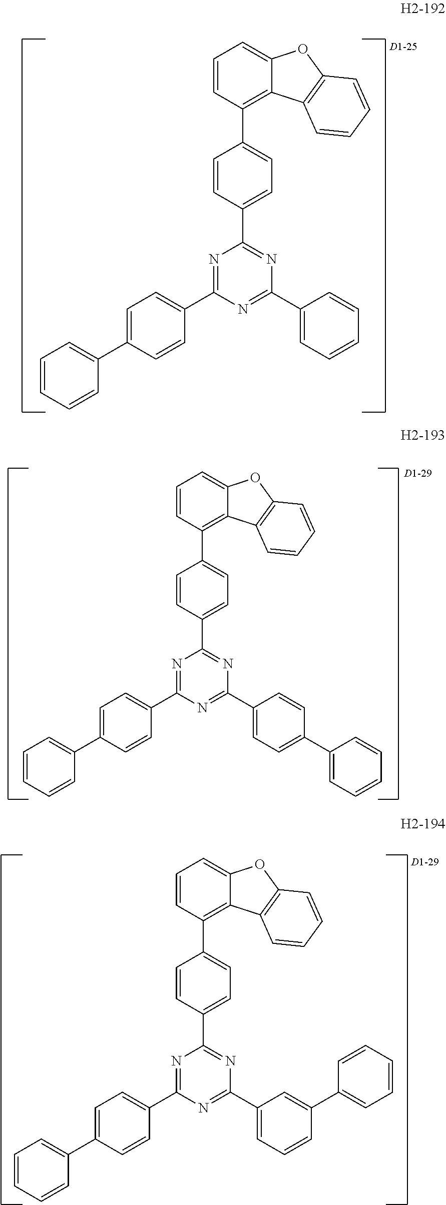

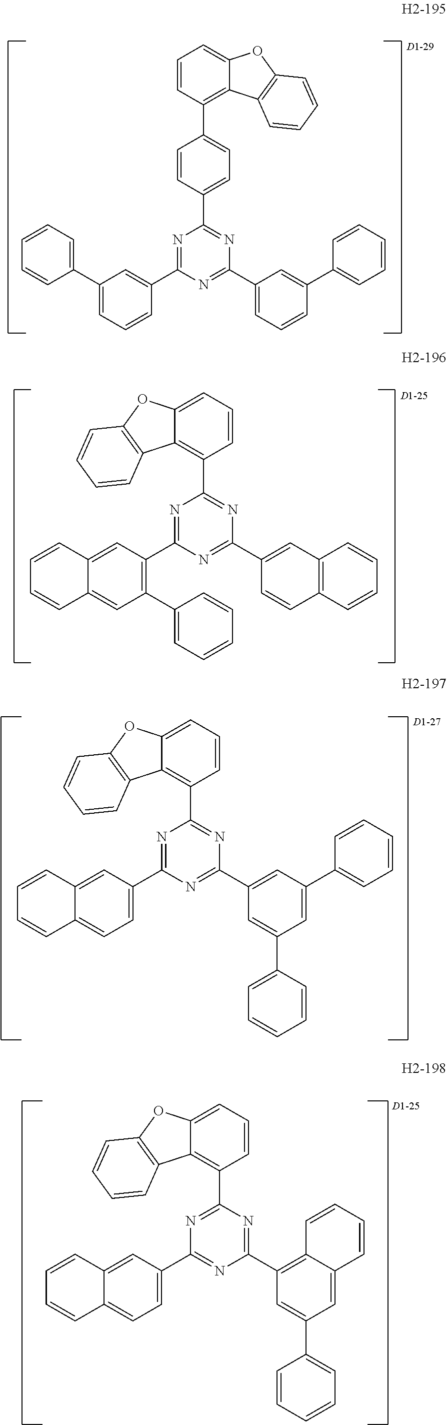

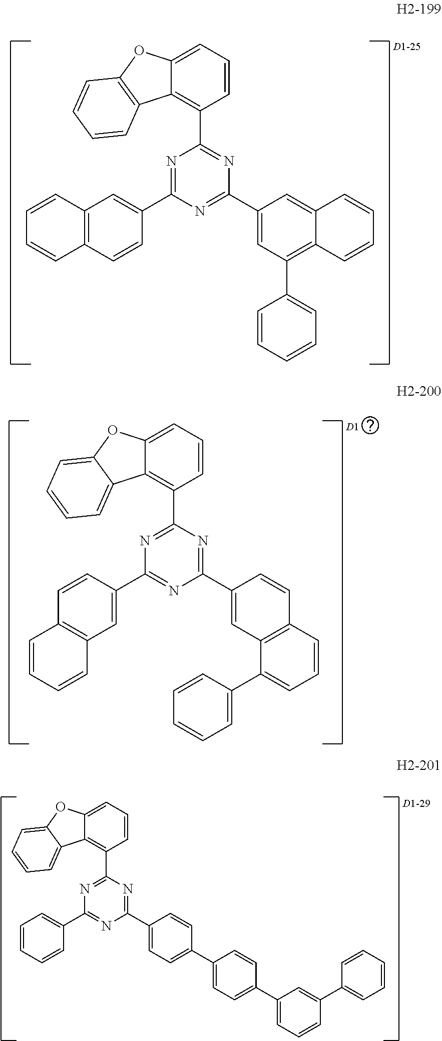

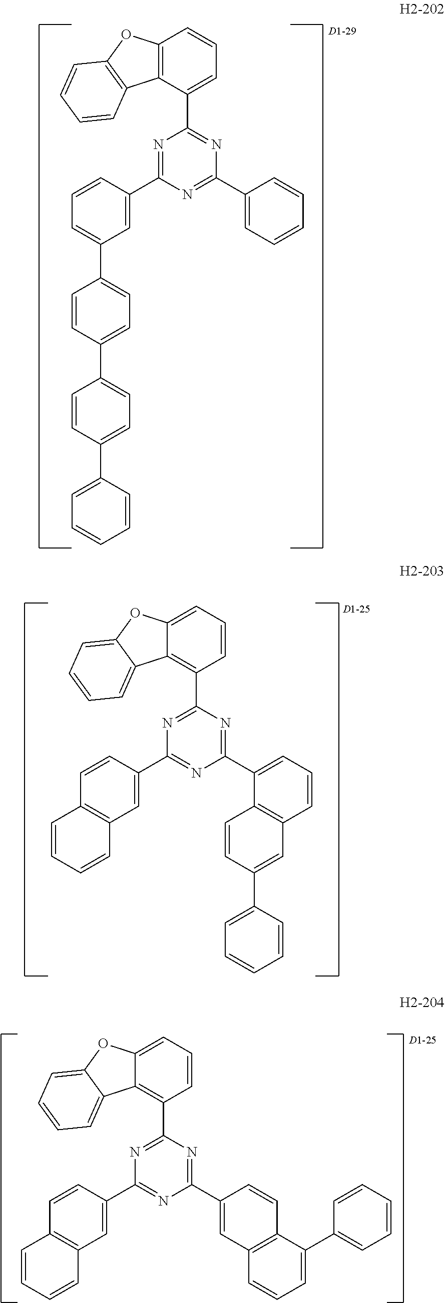

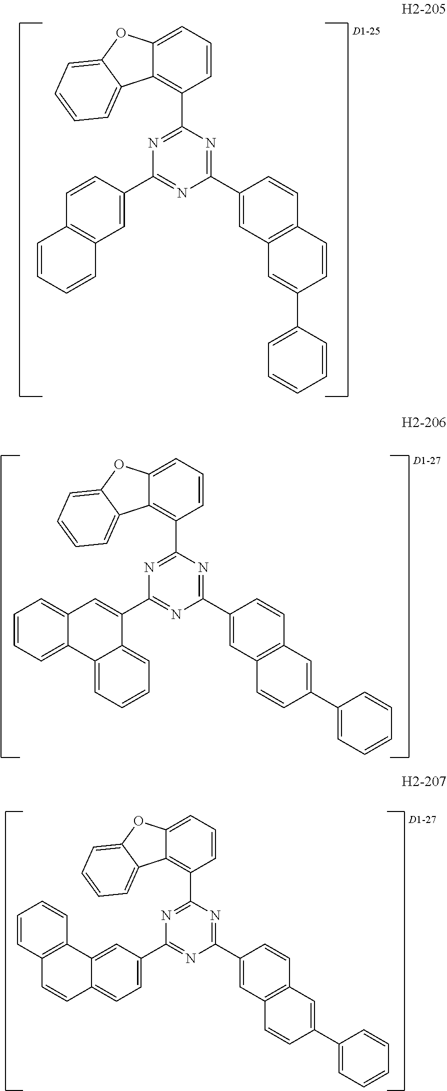

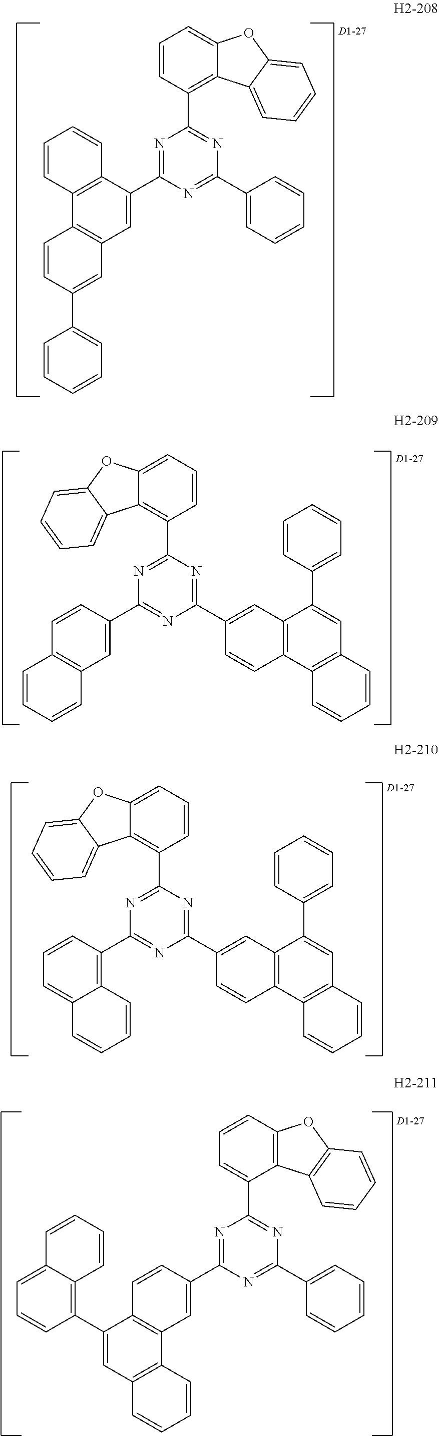

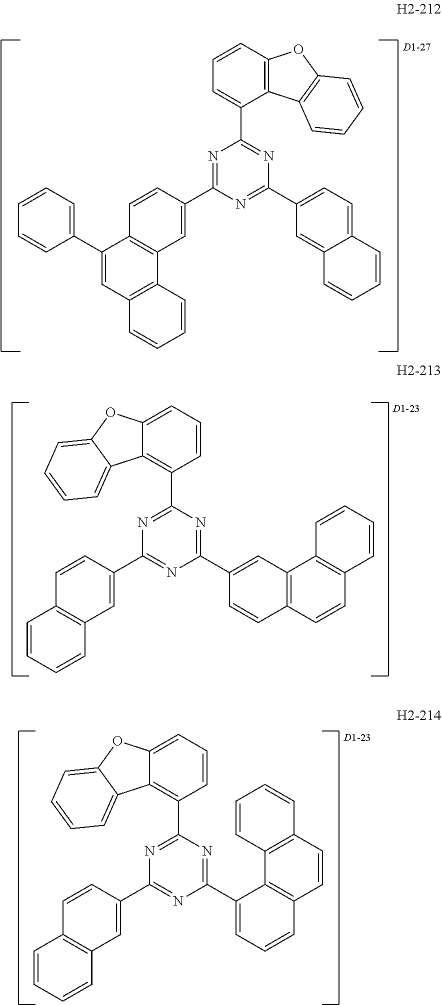

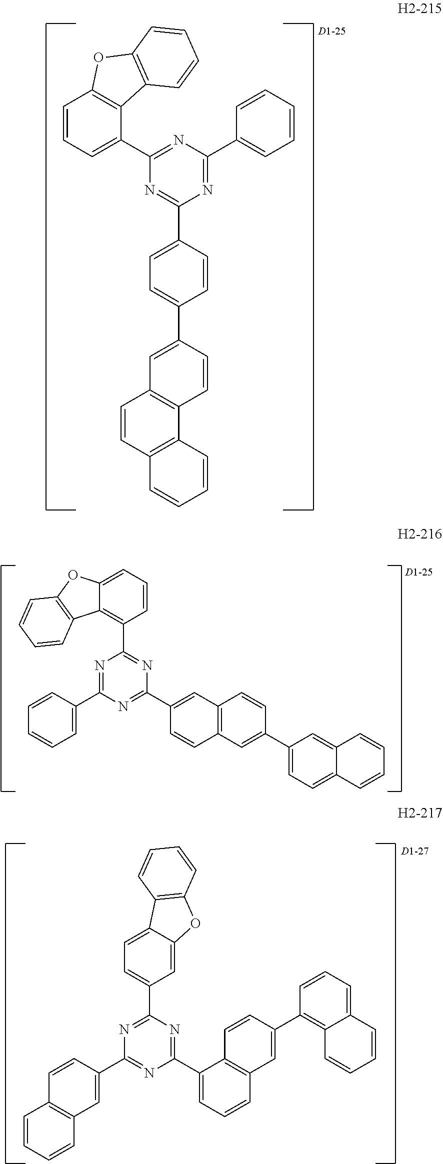

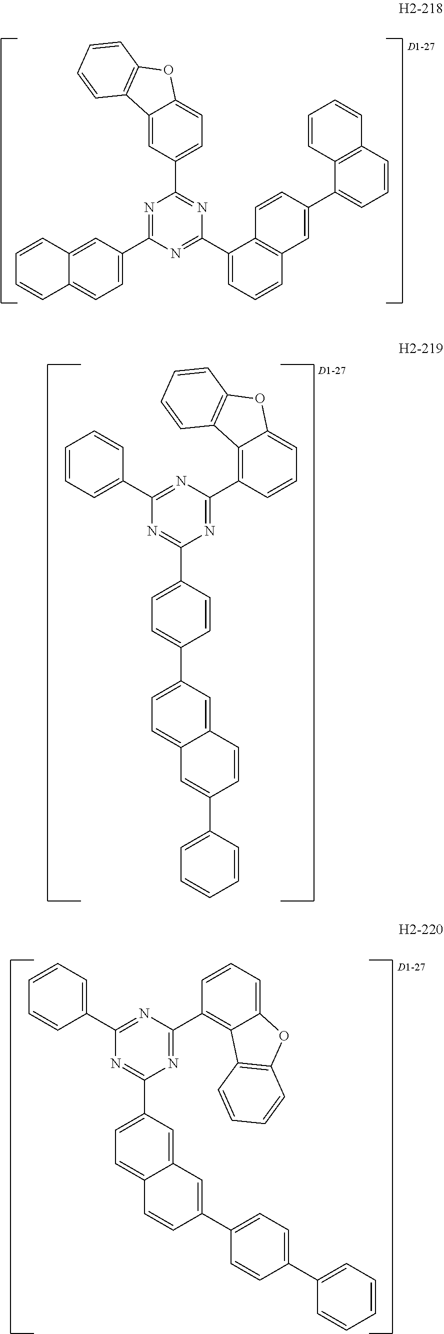

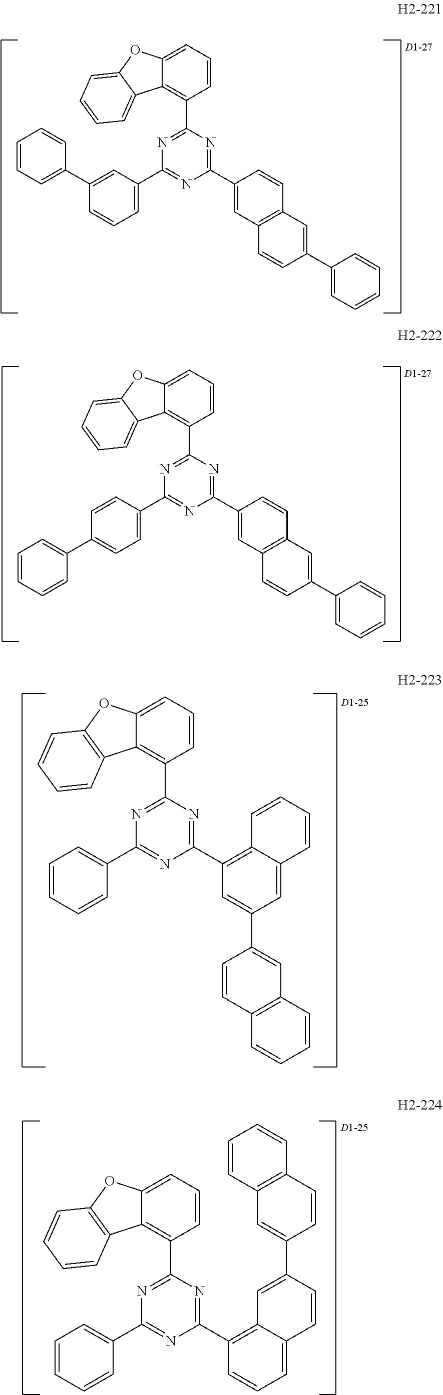

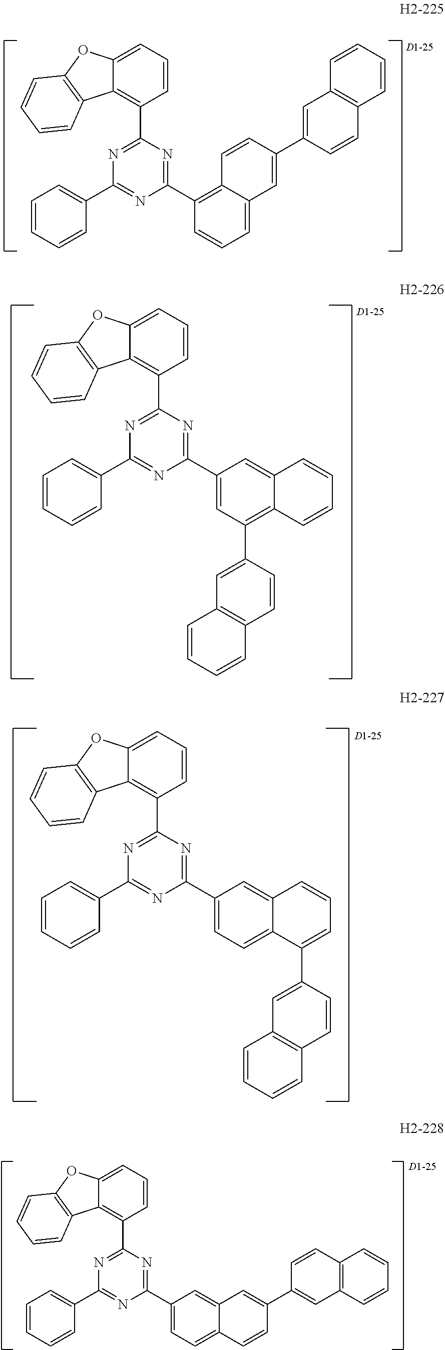

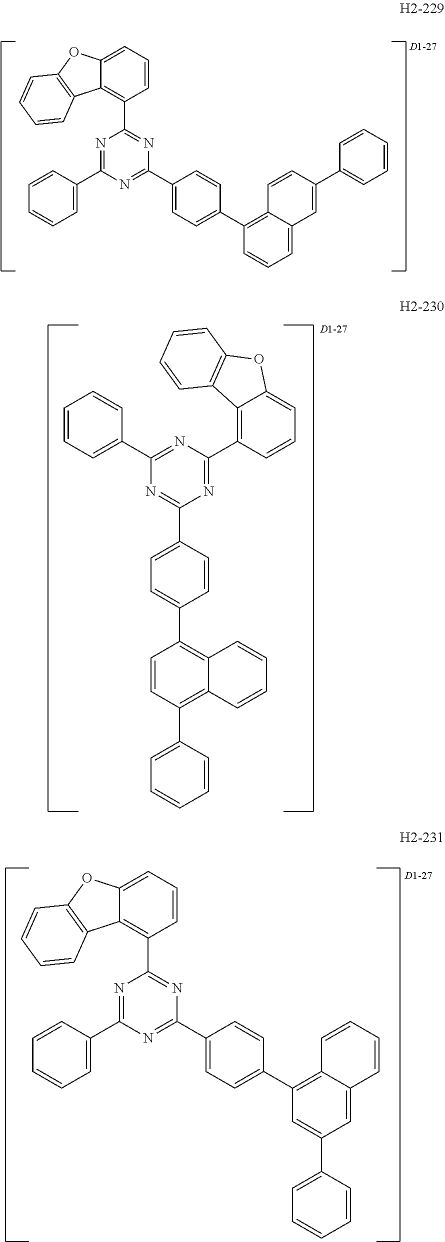

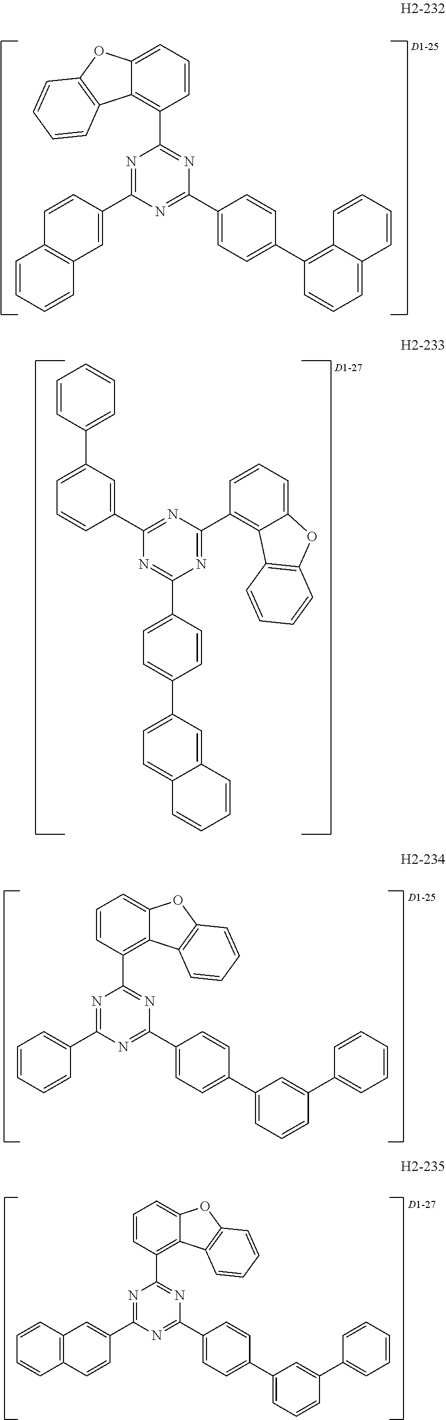

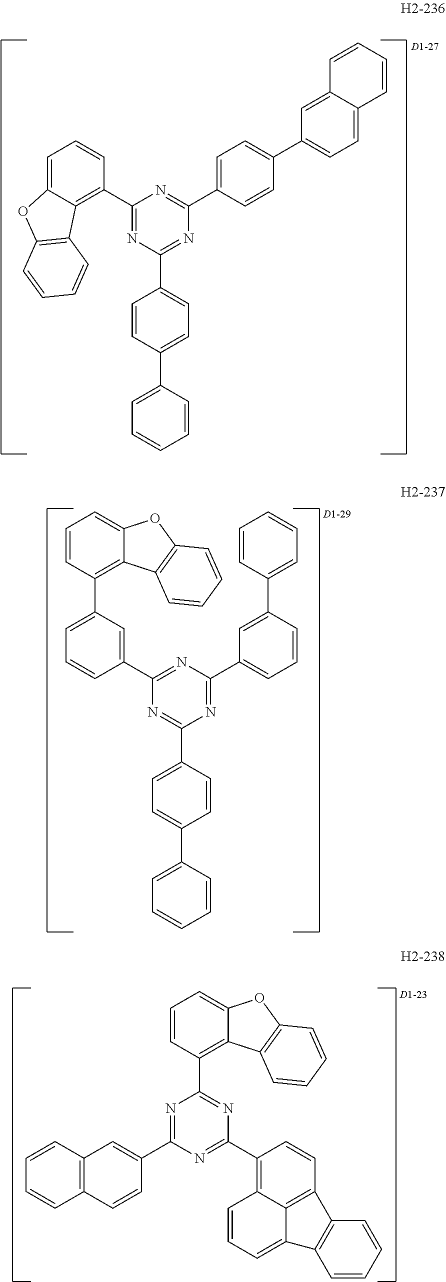

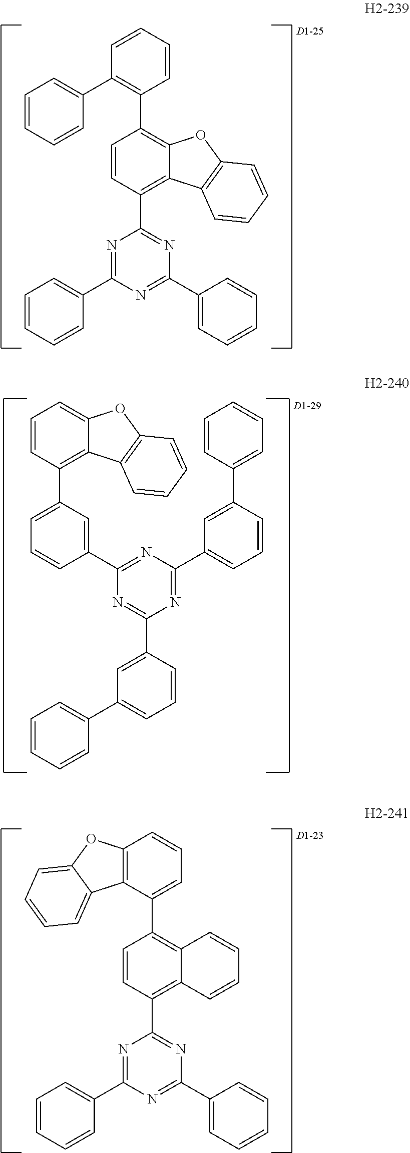

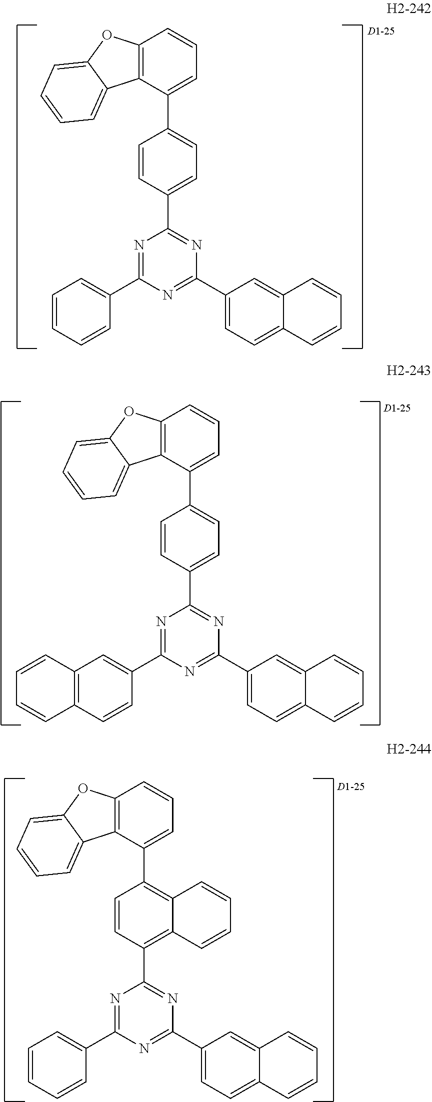

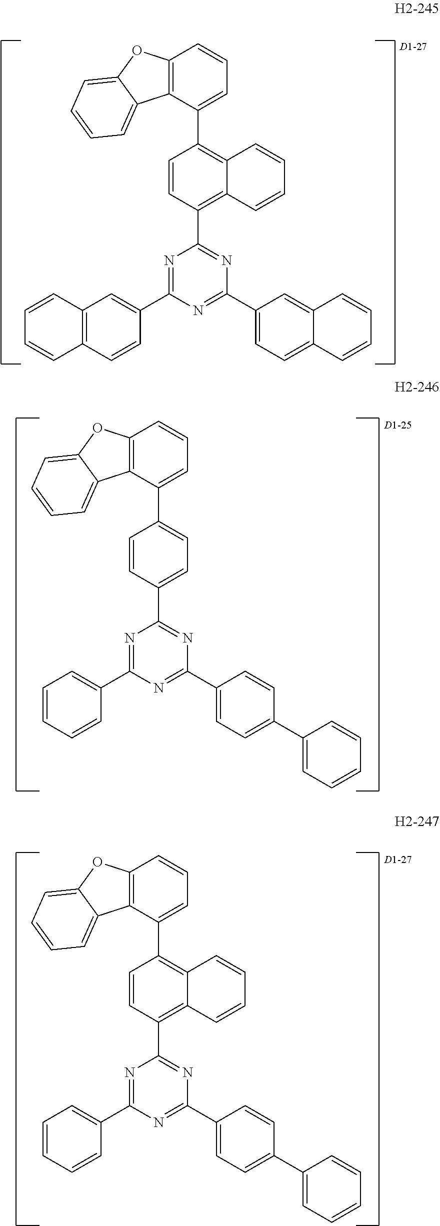

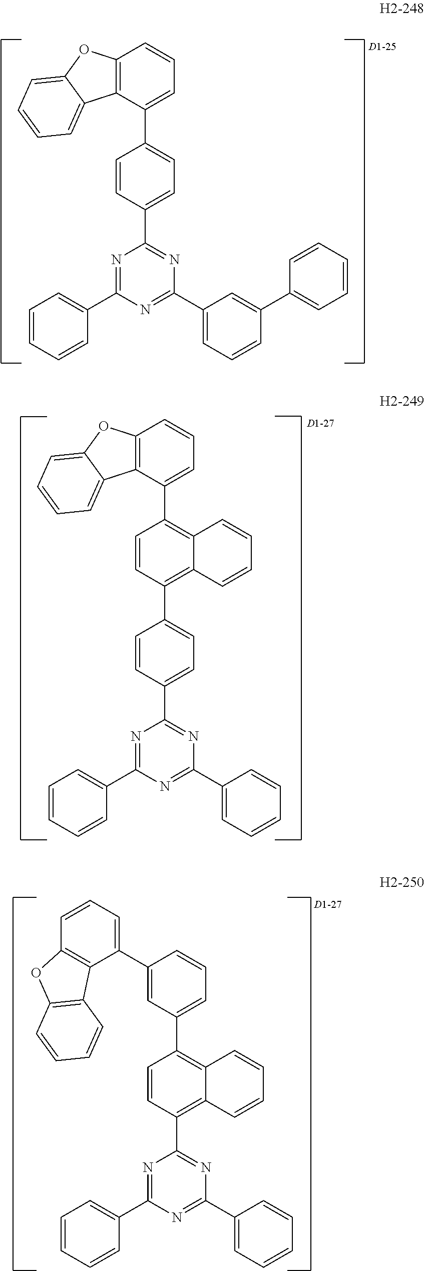

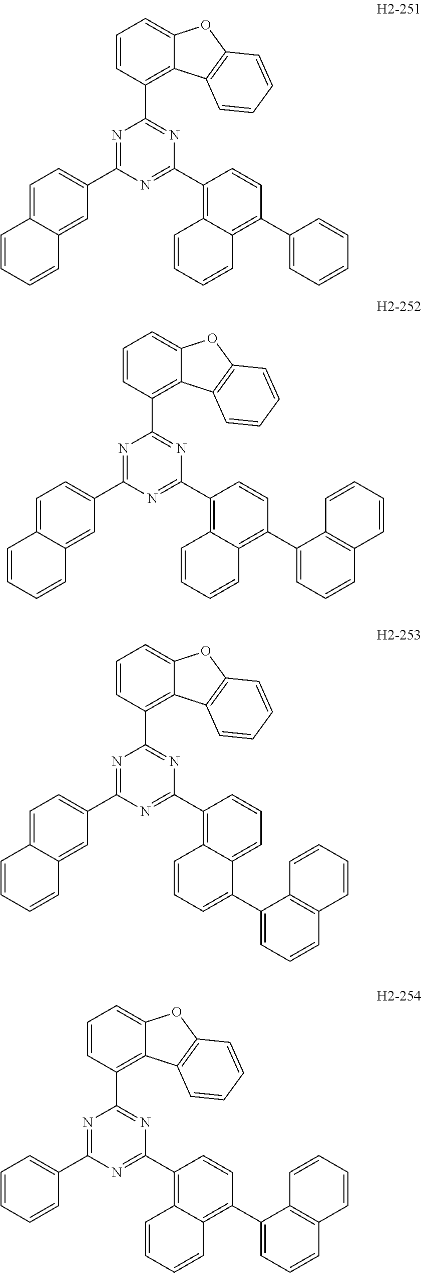 [SMILES: c1ccc(-c2ccc(-c3nc(-c4ccc5ccccc5c4)nc(-c4cccc5oc6ccccc6c45)n3)c3ccccc23)cc1 ; c1ccc2cc(-c3nc(-c4ccc(-c5cccc6ccccc56)c5ccccc45)nc(-c4cccc5oc6ccccc6c45)n3)ccc2c1 ; c1ccc2cc(-c3nc(-c4cccc5c(-c6cccc7ccccc67)cccc45)nc(-c4cccc5oc6ccccc6c45)n3)ccc2c1 ; c1ccc(-c2nc(-c3ccc(-c4cccc5ccccc45)c4ccccc34)nc(-c3cccc4oc5ccccc5c34)n2)cc1]

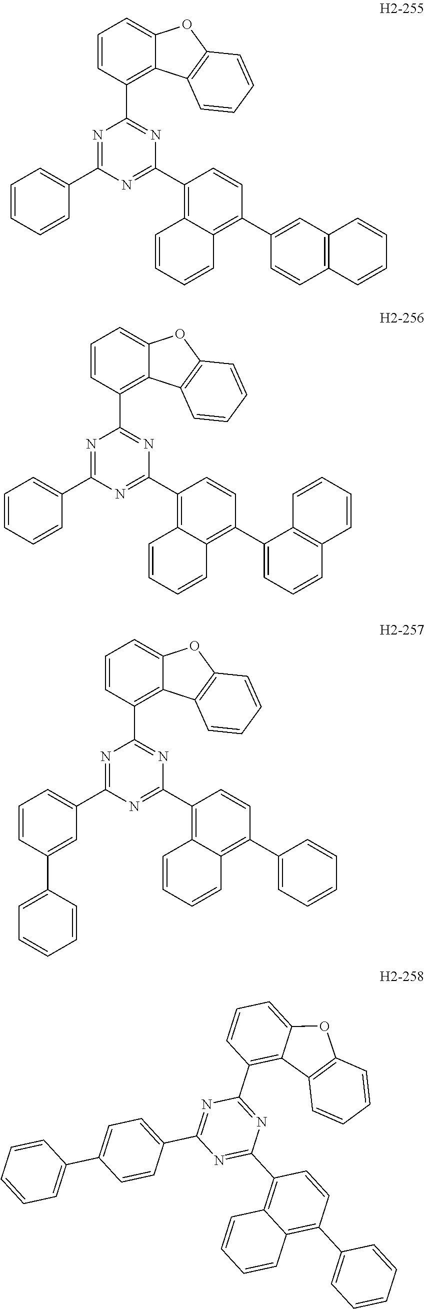 [SMILES: c1ccc(-c2nc(-c3ccc(-c4ccc5ccccc5c4)c4ccccc34)nc(-c3cccc4oc5ccccc5c34)n2)cc1 ; c1ccc(-c2nc(-c3ccc(-c4cccc5ccccc45)c4ccccc34)nc(-c3cccc4oc5ccccc5c34)n2)cc1 ; c1ccc(-c2cccc(-c3nc(-c4ccc(-c5ccccc5)c5ccccc45)nc(-c4cccc5oc6ccccc6c45)n3)c2)cc1 ; c1ccc(-c2ccc(-c3nc(-c4ccc(-c5ccccc5)c5ccccc45)nc(-c4cccc5oc6ccccc6c45)n3)cc2)cc1]

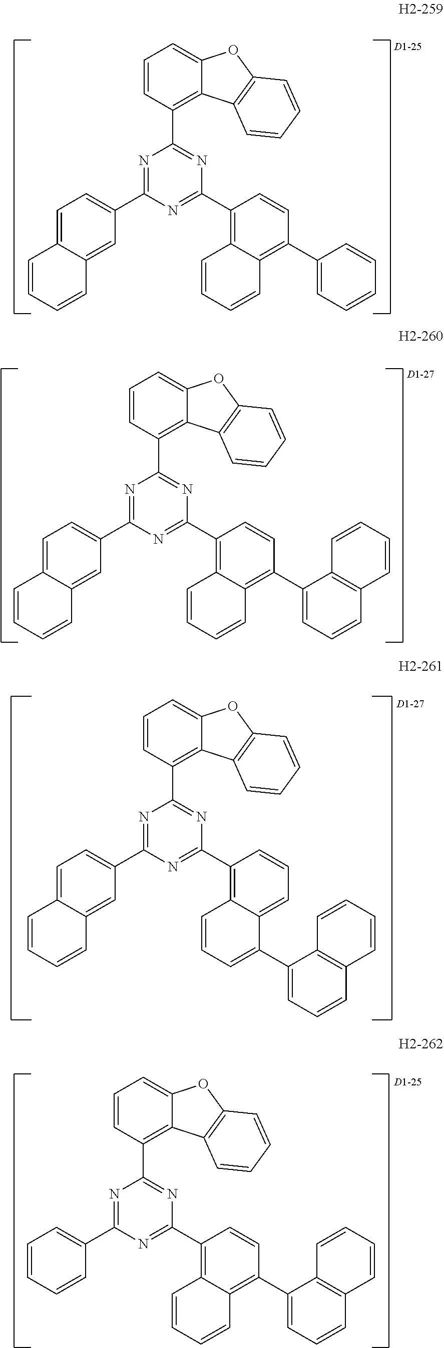

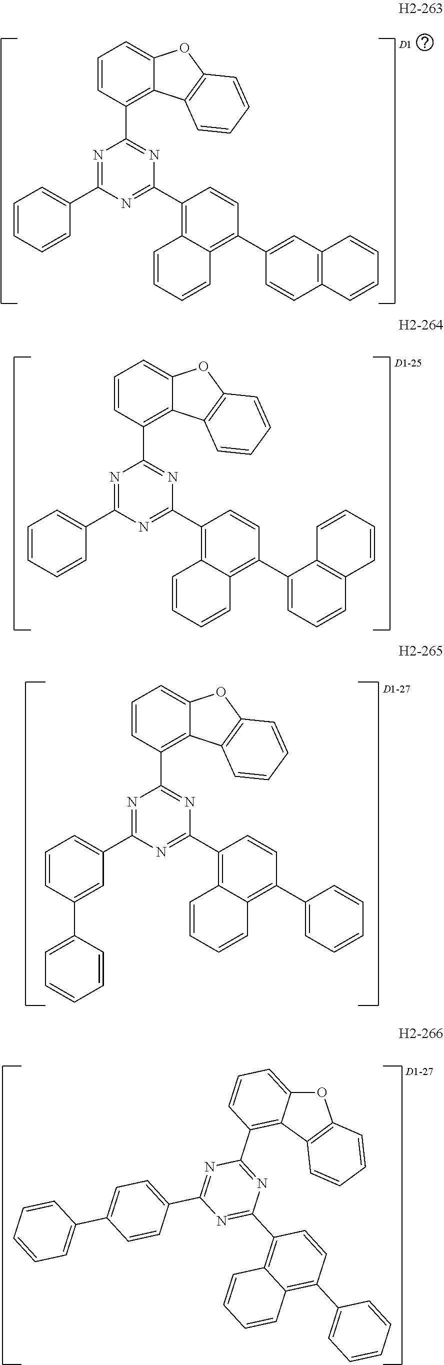

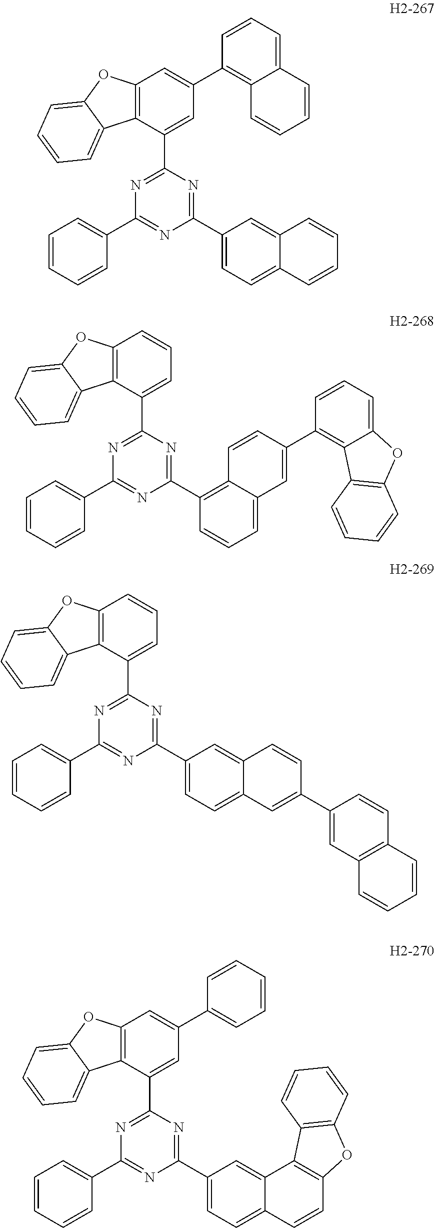 [SMILES: c1ccc(-c2nc(-c3ccc4ccccc4c3)nc(-c3cc(-c4cccc5ccccc45)cc4oc5ccccc5c34)n2)cc1 ; c1ccc(-c2nc(-c3cccc4cc(-c5cccc6oc7ccccc7c56)ccc34)nc(-c3cccc4oc5ccccc5c34)n2)cc1 ; c1ccc(-c2nc(-c3ccc4cc(-c5ccc6ccccc6c5)ccc4c3)nc(-c3cccc4oc5ccccc5c34)n2)cc1 ; c1ccc(-c2cc(-c3nc(-c4ccccc4)nc(-c4ccc5ccc6oc7ccccc7c6c5c4)n3)c3c(c2)oc2ccccc23)cc1]

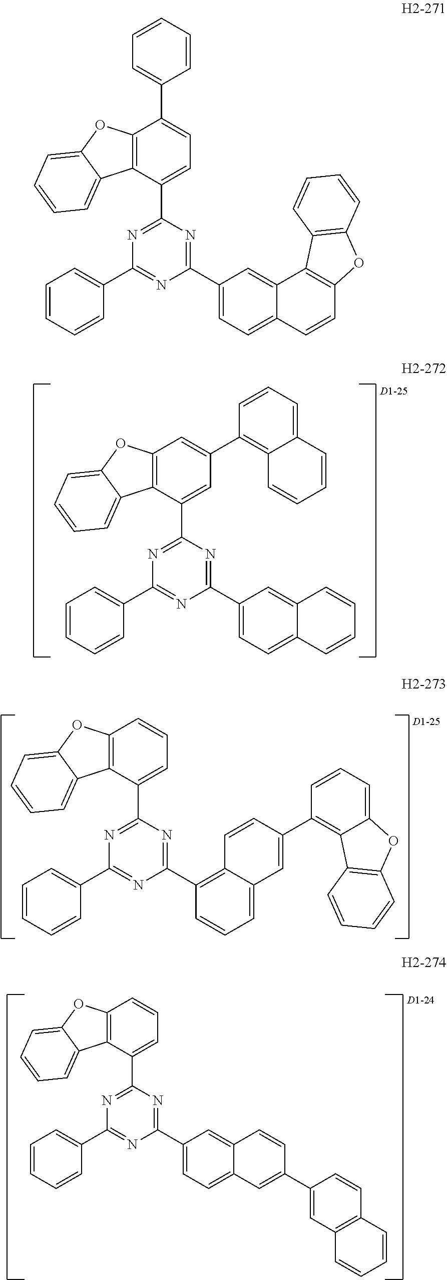

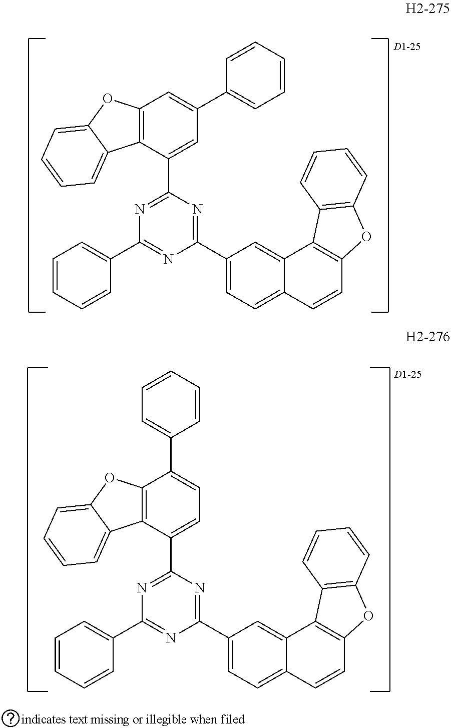

The compound represented by formula 3 may be selected from the group consisting of the following compounds, but is not limited thereto.

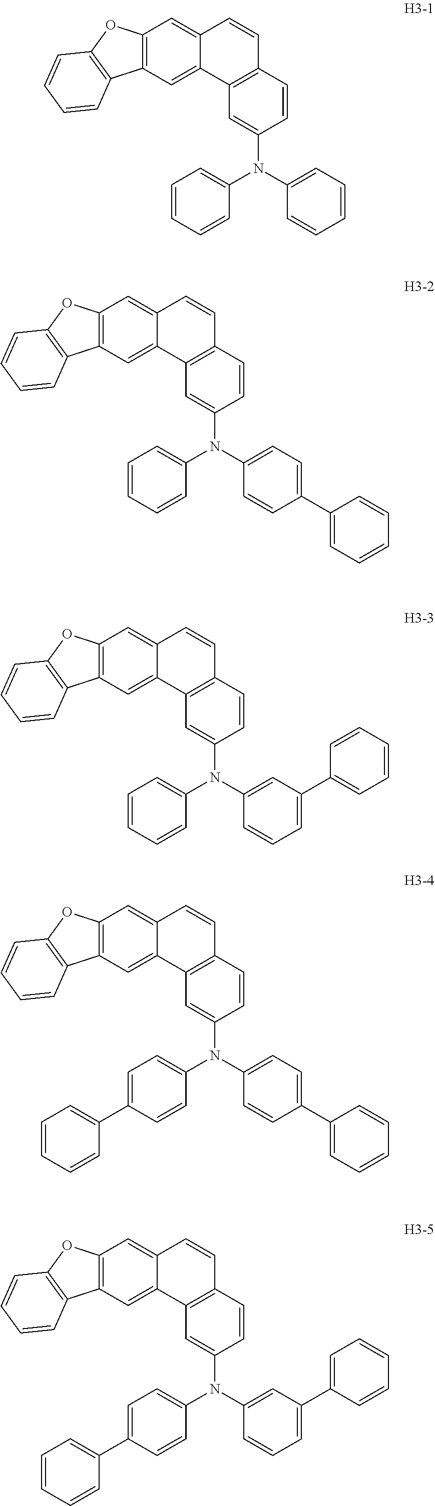 [SMILES: c1ccc(N(c2ccccc2)c2ccc3ccc4cc5oc6ccccc6c5cc4c3c2)cc1 ; c1ccc(-c2ccc(N(c3ccccc3)c3ccc4ccc5cc6oc7ccccc7c6cc5c4c3)cc2)cc1 ; c1ccc(-c2cccc(N(c3ccccc3)c3ccc4ccc5cc6oc7ccccc7c6cc5c4c3)c2)cc1 ; c1ccc(-c2ccc(N(c3ccc(-c4ccccc4)cc3)c3ccc4ccc5cc6oc7ccccc7c6cc5c4c3)cc2)cc1 ; c1ccc(-c2ccc(N(c3cccc(-c4ccccc4)c3)c3ccc4ccc5cc6oc7ccccc7c6cc5c4c3)cc2)cc1]

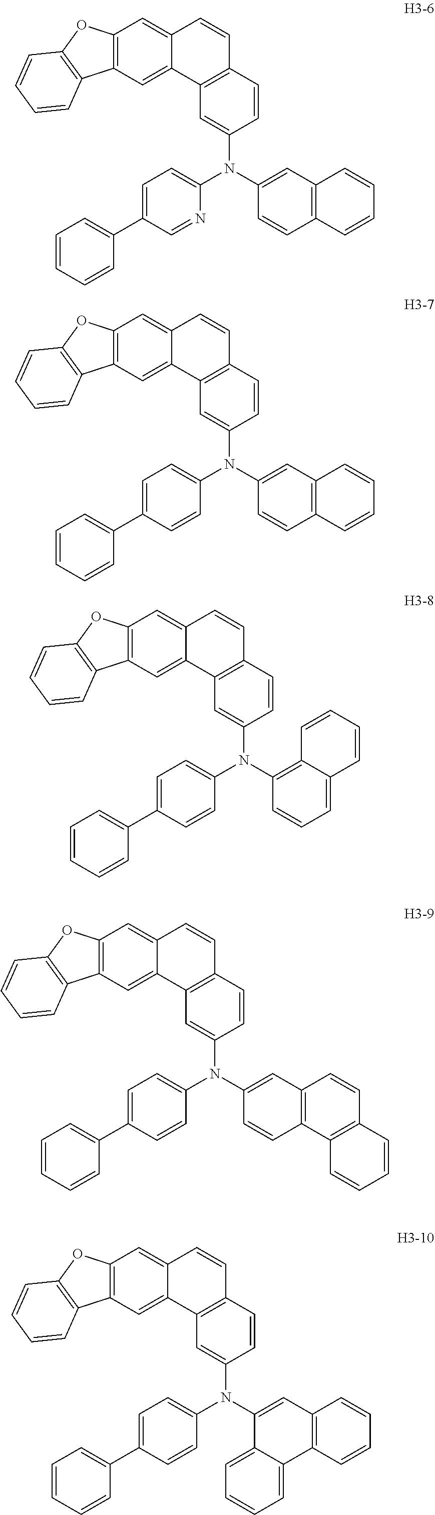

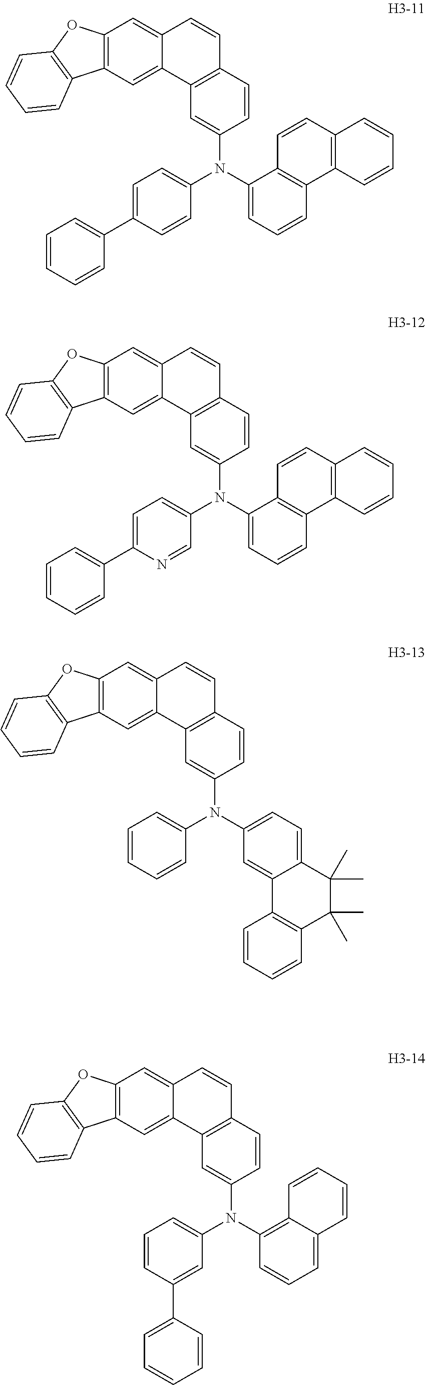 [SMILES: c1ccc(-c2ccc(N(c3ccc4ccc5cc6oc7ccccc7c6cc5c4c3)c3cccc4c3ccc3ccccc34)cc2)cc1 ; c1ccc(-c2ccc(N(c3ccc4ccc5cc6oc7ccccc7c6cc5c4c3)c3cccc4c3ccc3ccccc34)cn2)cc1 ; CC1(C)c2ccccc2-c2cc(N(c3ccccc3)c3ccc4ccc5cc6oc7ccccc7c6cc5c4c3)ccc2C1(C)C ; c1ccc(-c2cccc(N(c3ccc4ccc5cc6oc7ccccc7c6cc5c4c3)c3cccc4ccccc34)c2)cc1]

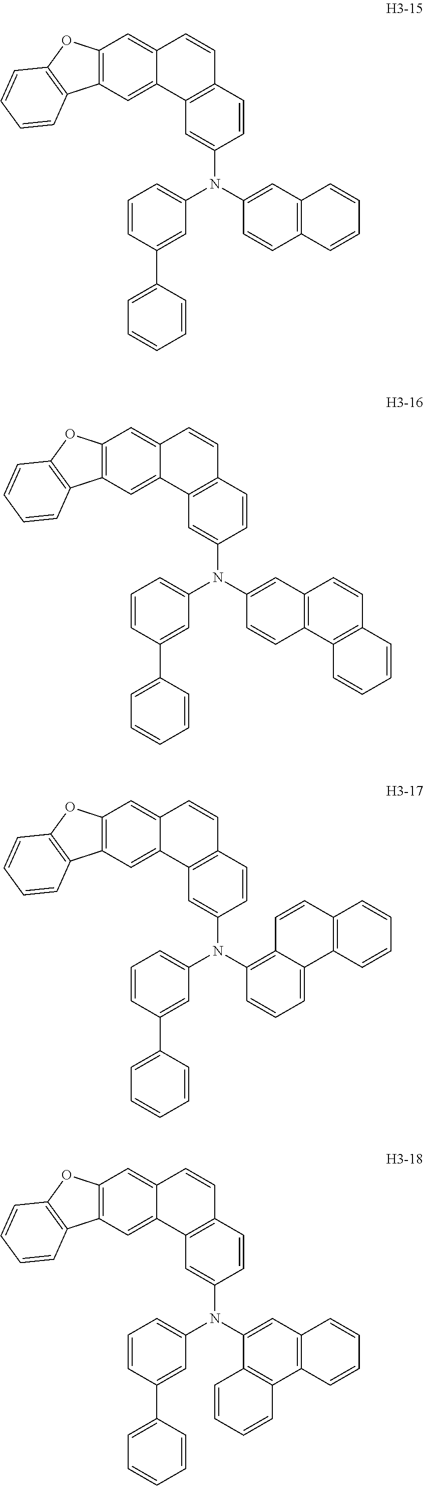 [SMILES: c1ccc(-c2cccc(N(c3ccc4ccccc4c3)c3ccc4ccc5cc6oc7ccccc7c6cc5c4c3)c2)cc1 ; c1ccc(-c2cccc(N(c3ccc4c(ccc5ccccc54)c3)c3ccc4ccc5cc6oc7ccccc7c6cc5c4c3)c2)cc1 ; c1ccc(-c2cccc(N(c3ccc4ccc5cc6oc7ccccc7c6cc5c4c3)c3cccc4c3ccc3ccccc34)c2)cc1 ; c1ccc(-c2cccc(N(c3ccc4ccc5cc6oc7ccccc7c6cc5c4c3)c3cc4ccccc4c4ccccc34)c2)cc1]

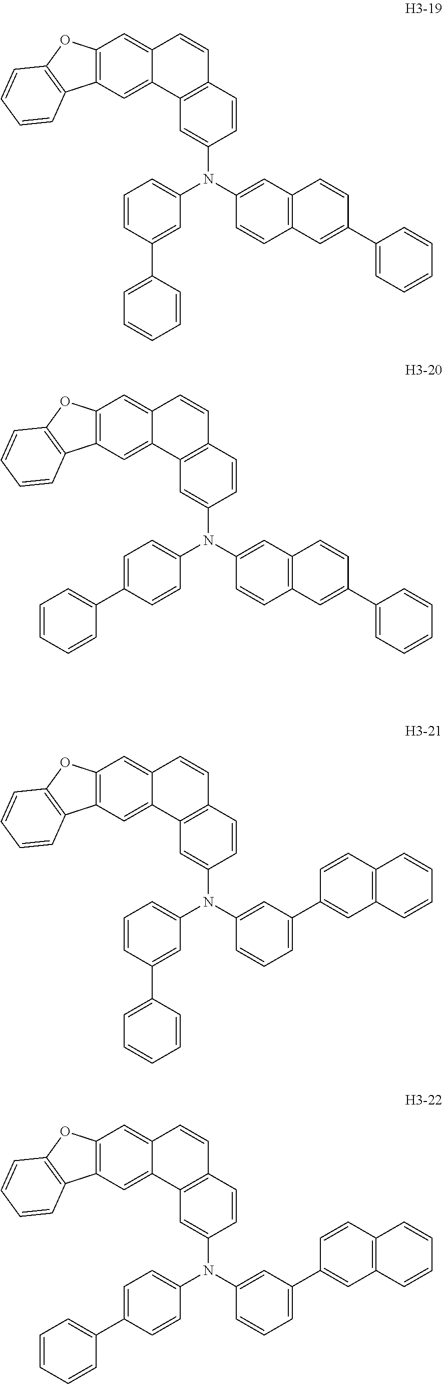 [SMILES: c1ccc(-c2cccc(N(c3ccc4cc(-c5ccccc5)ccc4c3)c3ccc4ccc5cc6oc7ccccc7c6cc5c4c3)c2)cc1 ; c1ccc(-c2ccc(N(c3ccc4cc(-c5ccccc5)ccc4c3)c3ccc4ccc5cc6oc7ccccc7c6cc5c4c3)cc2)cc1 ; c1ccc(-c2cccc(N(c3cccc(-c4ccc5ccccc5c4)c3)c3ccc4ccc5cc6oc7ccccc7c6cc5c4c3)c2)cc1 ; c1ccc(-c2ccc(N(c3cccc(-c4ccc5ccccc5c4)c3)c3ccc4ccc5cc6oc7ccccc7c6cc5c4c3)cc2)cc1]

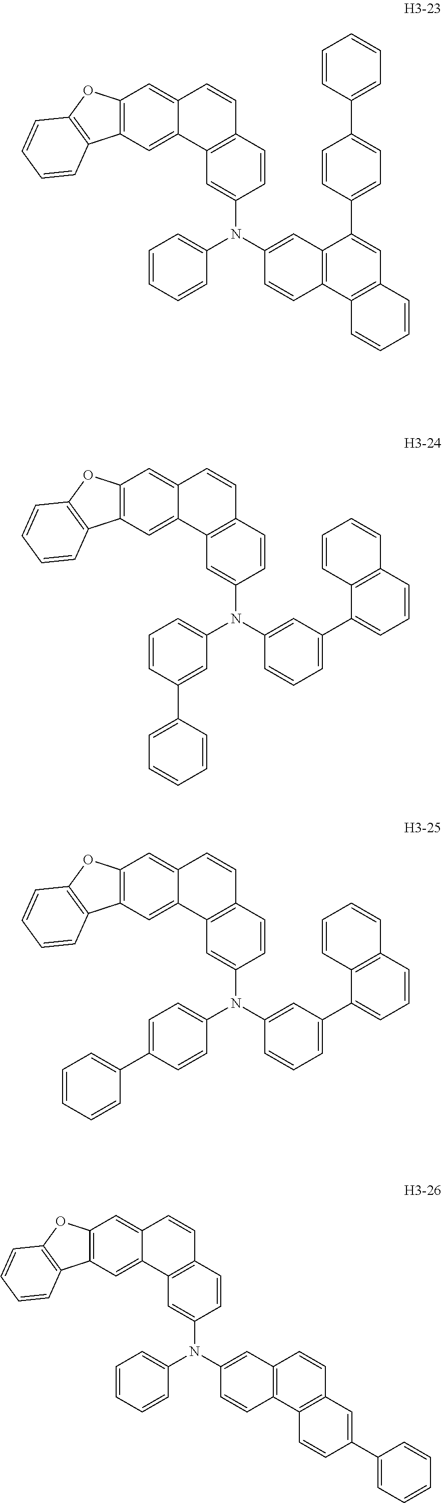 [SMILES: c1ccc(-c2ccc(-c3cc4ccccc4c4ccc(N(c5ccccc5)c5ccc6ccc7cc8oc9ccccc9c8cc7c6c5)cc34)cc2)cc1 ; c1ccc(-c2cccc(N(c3cccc(-c4cccc5ccccc45)c3)c3ccc4ccc5cc6oc7ccccc7c6cc5c4c3)c2)cc1 ; c1ccc(-c2ccc(N(c3cccc(-c4cccc5ccccc45)c3)c3ccc4ccc5cc6oc7ccccc7c6cc5c4c3)cc2)cc1 ; c1ccc(-c2ccc3c(ccc4cc(N(c5ccccc5)c5ccc6ccc7cc8oc9ccccc9c8cc7c6c5)ccc43)c2)cc1]

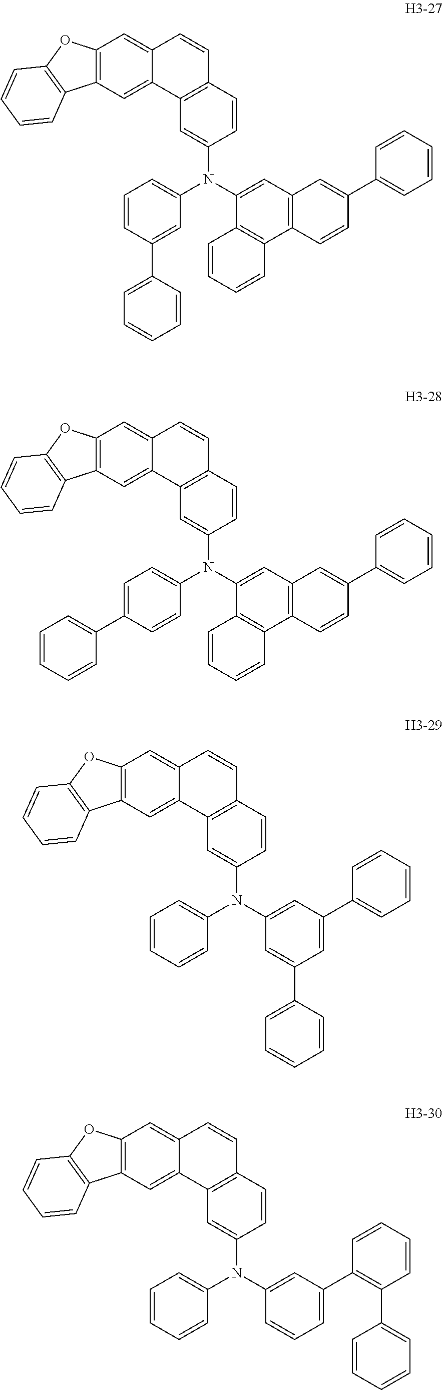 [SMILES: c1ccc(-c2cccc(N(c3ccc4ccc5cc6oc7ccccc7c6cc5c4c3)c3cc4cc(-c5ccccc5)ccc4c4ccccc34)c2)cc1 ; c1ccc(-c2ccc(N(c3ccc4ccc5cc6oc7ccccc7c6cc5c4c3)c3cc4cc(-c5ccccc5)ccc4c4ccccc34)cc2)cc1 ; c1ccc(-c2cc(-c3ccccc3)cc(N(c3ccccc3)c3ccc4ccc5cc6oc7ccccc7c6cc5c4c3)c2)cc1 ; c1ccc(-c2ccccc2-c2cccc(N(c3ccccc3)c3ccc4ccc5cc6oc7ccccc7c6cc5c4c3)c2)cc1]

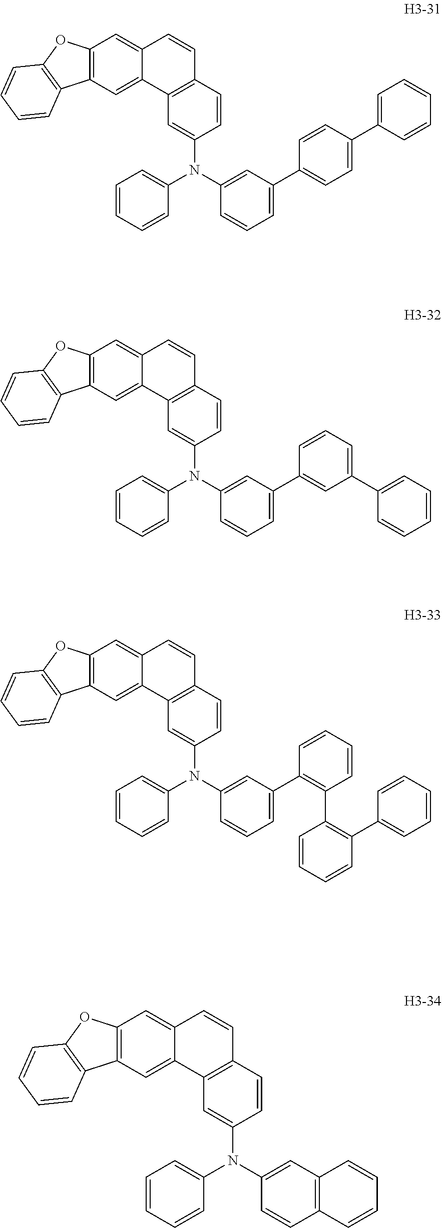 [SMILES: c1ccc(-c2ccc(-c3cccc(N(c4ccccc4)c4ccc5ccc6cc7oc8ccccc8c7cc6c5c4)c3)cc2)cc1 ; c1ccc(-c2cccc(-c3cccc(N(c4ccccc4)c4ccc5ccc6cc7oc8ccccc8c7cc6c5c4)c3)c2)cc1 ; c1ccc(-c2ccccc2-c2ccccc2-c2cccc(N(c3ccccc3)c3ccc4ccc5cc6oc7ccccc7c6cc5c4c3)c2)cc1 ; c1ccc(N(c2ccc3ccccc3c2)c2ccc3ccc4cc5oc6ccccc6c5cc4c3c2)cc1]

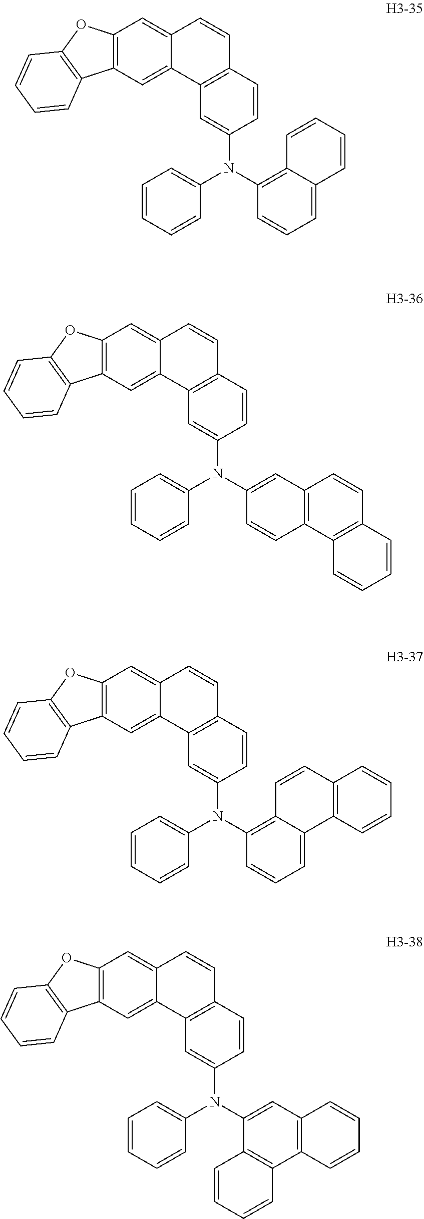 [SMILES: c1ccc(N(c2ccc3ccc4cc5oc6ccccc6c5cc4c3c2)c2cccc3ccccc23)cc1 ; c1ccc(N(c2ccc3c(ccc4ccccc43)c2)c2ccc3ccc4cc5oc6ccccc6c5cc4c3c2)cc1 ; c1ccc(N(c2ccc3ccc4cc5oc6ccccc6c5cc4c3c2)c2cccc3c2ccc2ccccc23)cc1 ; c1ccc(N(c2ccc3ccc4cc5oc6ccccc6c5cc4c3c2)c2cc3ccccc3c3ccccc23)cc1]

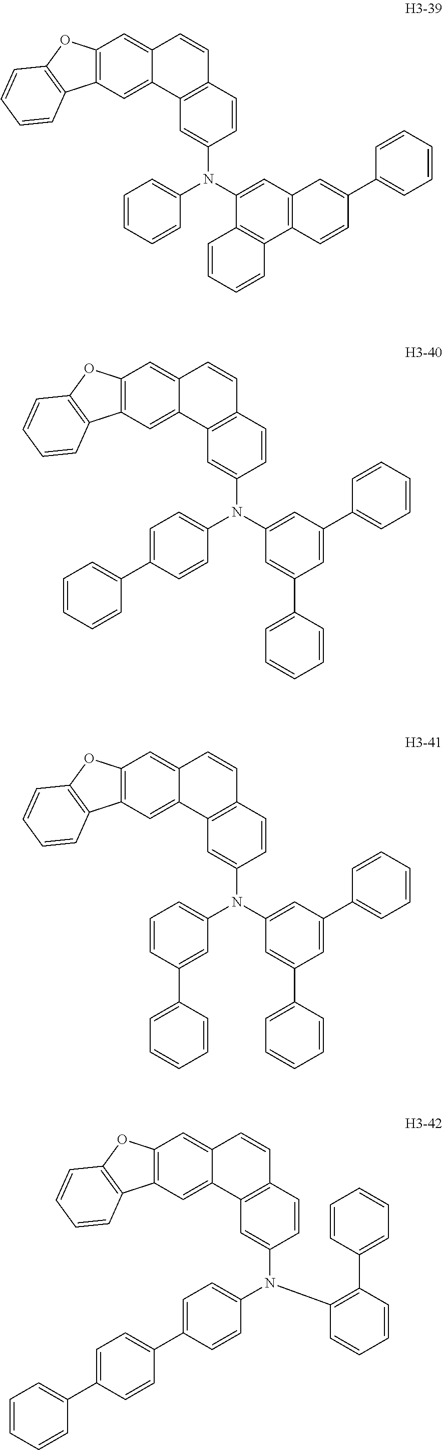 [SMILES: c1ccc(-c2ccc3c(c2)cc(N(c2ccccc2)c2ccc4ccc5cc6oc7ccccc7c6cc5c4c2)c2ccccc23)cc1 ; c1ccc(-c2ccc(N(c3cc(-c4ccccc4)cc(-c4ccccc4)c3)c3ccc4ccc5cc6oc7ccccc7c6cc5c4c3)cc2)cc1 ; c1ccc(-c2cccc(N(c3cc(-c4ccccc4)cc(-c4ccccc4)c3)c3ccc4ccc5cc6oc7ccccc7c6cc5c4c3)c2)cc1 ; c1ccc(-c2ccc(-c3ccc(N(c4ccc5ccc6cc7oc8ccccc8c7cc6c5c4)c4ccccc4-c4ccccc4)cc3)cc2)cc1]

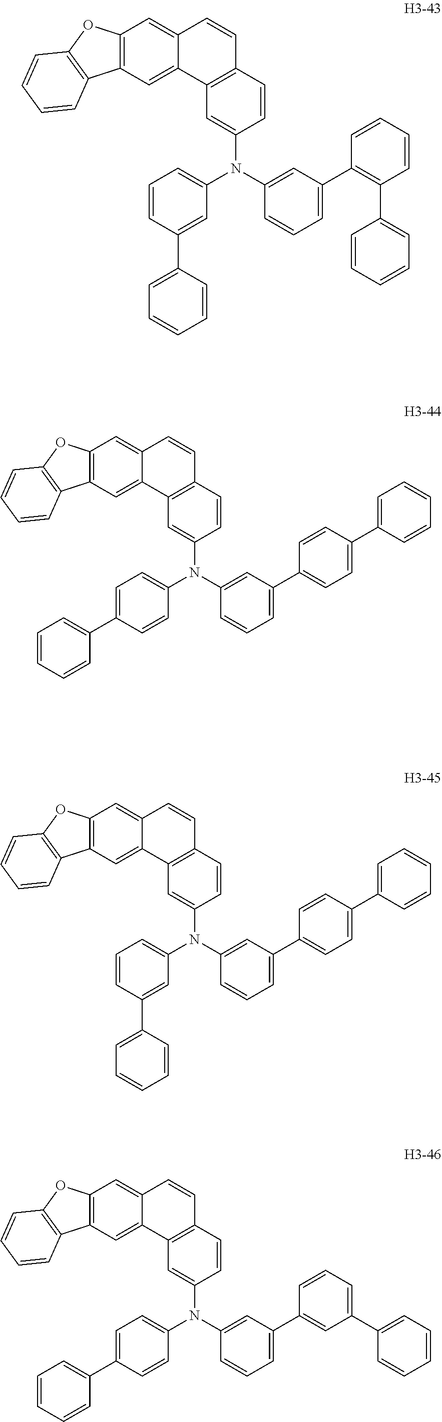 [SMILES: c1ccc(-c2cccc(N(c3cccc(-c4ccccc4-c4ccccc4)c3)c3ccc4ccc5cc6oc7ccccc7c6cc5c4c3)c2)cc1 ; c1ccc(-c2ccc(-c3cccc(N(c4ccc(-c5ccccc5)cc4)c4ccc5ccc6cc7oc8ccccc8c7cc6c5c4)c3)cc2)cc1 ; c1ccc(-c2ccc(-c3cccc(N(c4cccc(-c5ccccc5)c4)c4ccc5ccc6cc7oc8ccccc8c7cc6c5c4)c3)cc2)cc1 ; c1ccc(-c2ccc(N(c3cccc(-c4cccc(-c5ccccc5)c4)c3)c3ccc4ccc5cc6oc7ccccc7c6cc5c4c3)cc2)cc1]

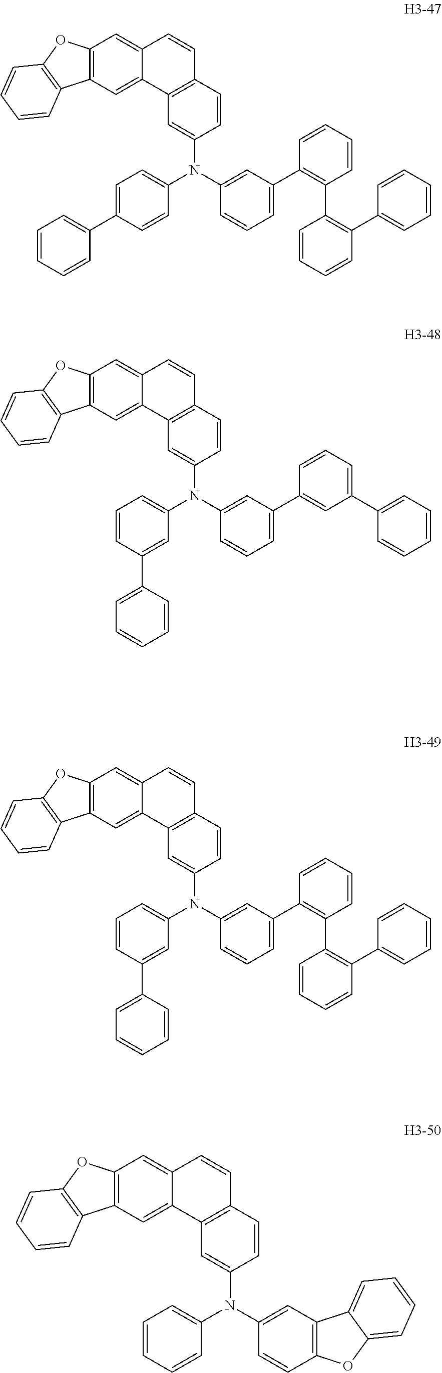 [SMILES: c1ccc(-c2ccc(N(c3cccc(-c4ccccc4-c4ccccc4-c4ccccc4)c3)c3ccc4ccc5cc6oc7ccccc7c6cc5c4c3)cc2)cc1 ; c1ccc(-c2cccc(-c3cccc(N(c4cccc(-c5ccccc5)c4)c4ccc5ccc6cc7oc8ccccc8c7cc6c5c4)c3)c2)cc1 ; c1ccc(-c2cccc(N(c3cccc(-c4ccccc4-c4ccccc4-c4ccccc4)c3)c3ccc4ccc5cc6oc7ccccc7c6cc5c4c3)c2)cc1 ; c1ccc(N(c2ccc3ccc4cc5oc6ccccc6c5cc4c3c2)c2ccc3oc4ccccc4c3c2)cc1]

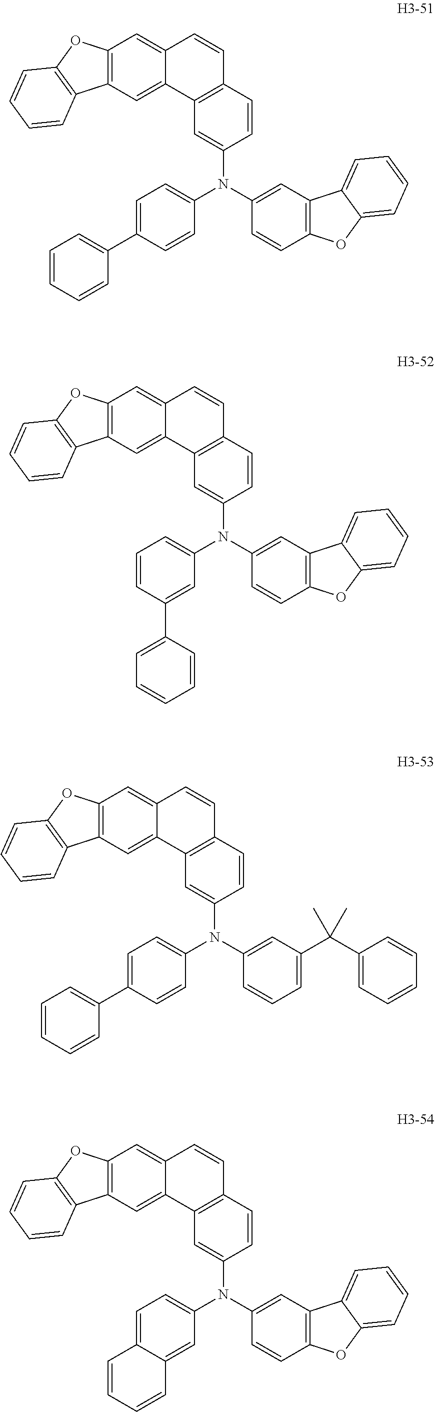 [SMILES: c1ccc(-c2ccc(N(c3ccc4ccc5cc6oc7ccccc7c6cc5c4c3)c3ccc4oc5ccccc5c4c3)cc2)cc1 ; c1ccc(-c2cccc(N(c3ccc4ccc5cc6oc7ccccc7c6cc5c4c3)c3ccc4oc5ccccc5c4c3)c2)cc1 ; CC(C)(c1ccccc1)c1cccc(N(c2ccc(-c3ccccc3)cc2)c2ccc3ccc4cc5oc6ccccc6c5cc4c3c2)c1 ; c1ccc2cc(N(c3ccc4ccc5cc6oc7ccccc7c6cc5c4c3)c3ccc4oc5ccccc5c4c3)ccc2c1]

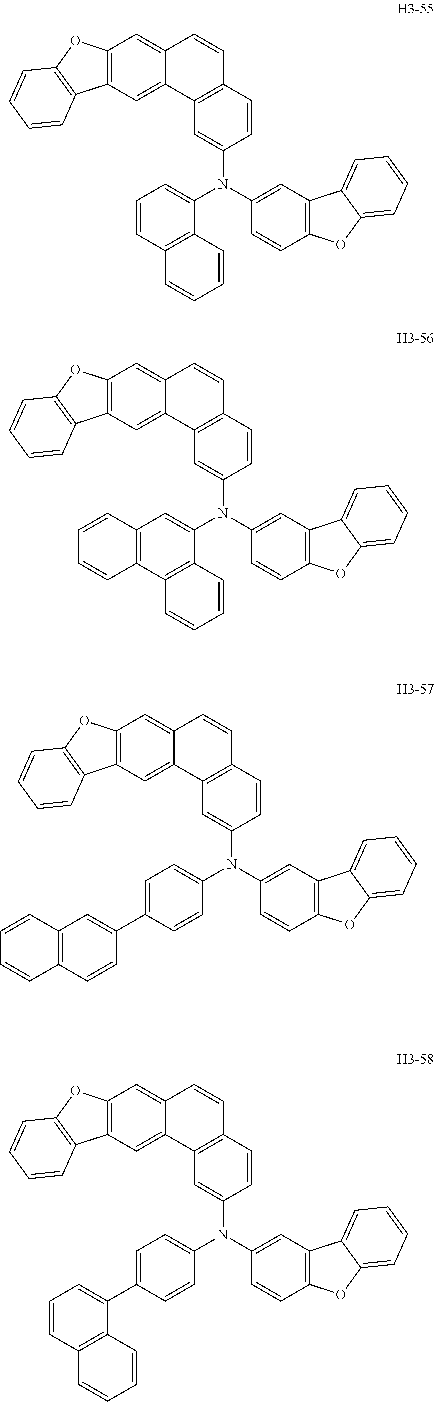 [SMILES: c1ccc2c(N(c3ccc4ccc5cc6oc7ccccc7c6cc5c4c3)c3ccc4oc5ccccc5c4c3)cccc2c1 ; c1ccc2c(c1)cc(N(c1ccc3ccc4cc5oc6ccccc6c5cc4c3c1)c1ccc3oc4ccccc4c3c1)c1ccccc12 ; c1ccc2cc(-c3ccc(N(c4ccc5ccc6cc7oc8ccccc8c7cc6c5c4)c4ccc5oc6ccccc6c5c4)cc3)ccc2c1 ; c1ccc2c(-c3ccc(N(c4ccc5ccc6cc7oc8ccccc8c7cc6c5c4)c4ccc5oc6ccccc6c5c4)cc3)cccc2c1]

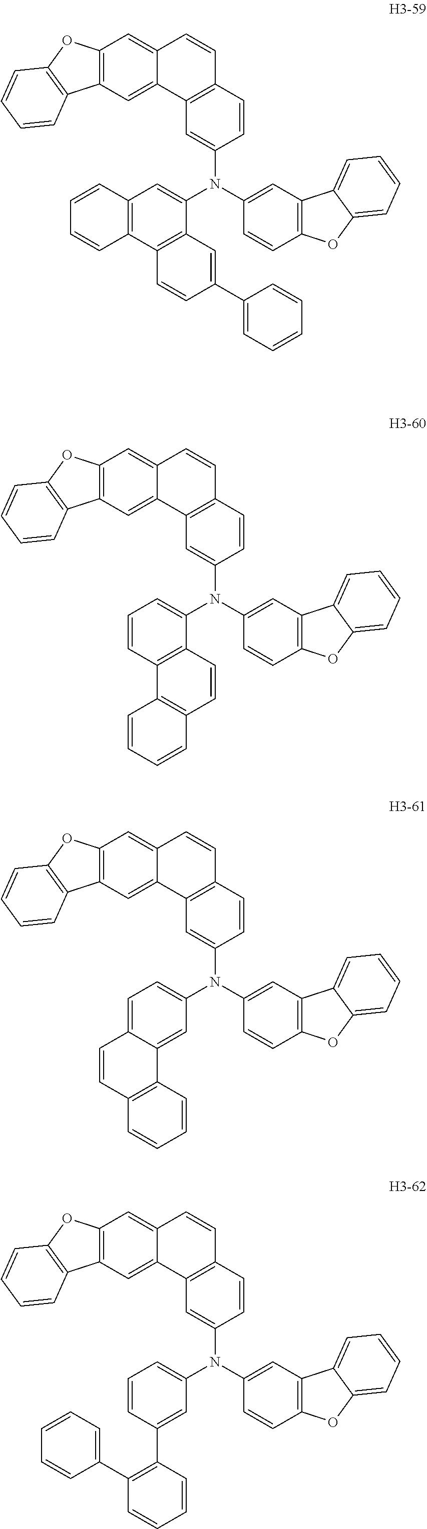 [SMILES: c1ccc(-c2ccc3c(c2)c(N(c2ccc4ccc5cc6oc7ccccc7c6cc5c4c2)c2ccc4oc5ccccc5c4c2)cc2ccccc23)cc1 ; c1ccc2c(c1)ccc1c(N(c3ccc4ccc5cc6oc7ccccc7c6cc5c4c3)c3ccc4oc5ccccc5c4c3)cccc12 ; c1ccc2c(c1)ccc1ccc(N(c3ccc4ccc5cc6oc7ccccc7c6cc5c4c3)c3ccc4oc5ccccc5c4c3)cc12 ; c1ccc(-c2ccccc2-c2cccc(N(c3ccc4ccc5cc6oc7ccccc7c6cc5c4c3)c3ccc4oc5ccccc5c4c3)c2)cc1]

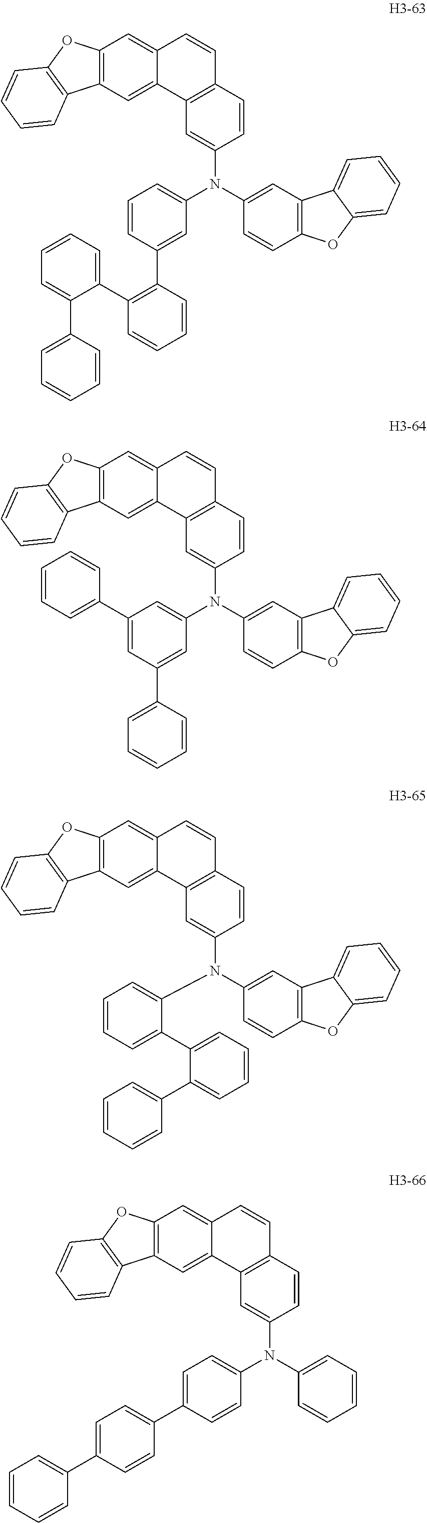 [SMILES: c1ccc(-c2ccccc2-c2ccccc2-c2cccc(N(c3ccc4ccc5cc6oc7ccccc7c6cc5c4c3)c3ccc4oc5ccccc5c4c3)c2)cc1 ; c1ccc(-c2cc(-c3ccccc3)cc(N(c3ccc4ccc5cc6oc7ccccc7c6cc5c4c3)c3ccc4oc5ccccc5c4c3)c2)cc1 ; c1ccc(-c2ccccc2-c2ccccc2N(c2ccc3ccc4cc5oc6ccccc6c5cc4c3c2)c2ccc3oc4ccccc4c3c2)cc1 ; c1ccc(-c2ccc(-c3ccc(N(c4ccccc4)c4ccc5ccc6cc7oc8ccccc8c7cc6c5c4)cc3)cc2)cc1]

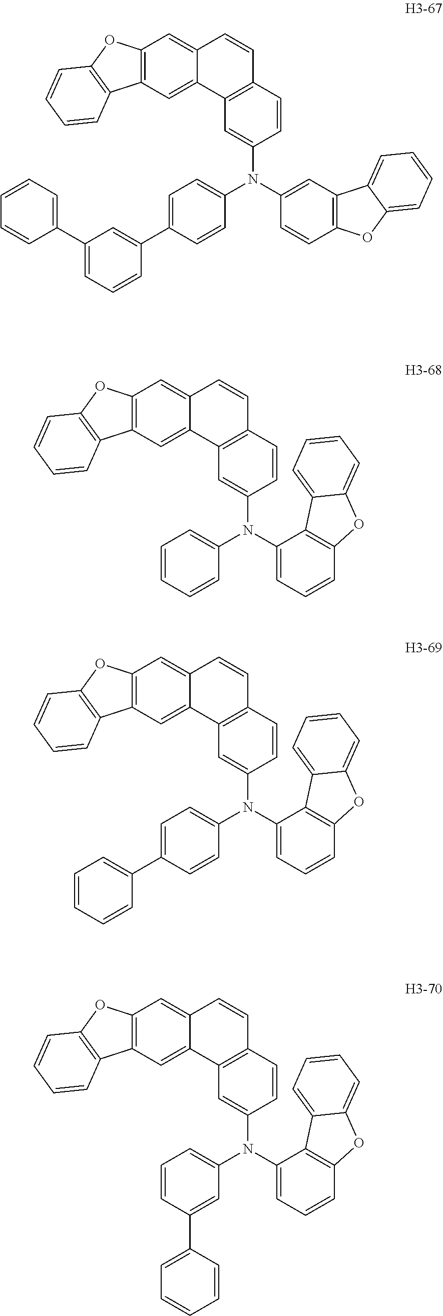 [SMILES: c1ccc(-c2cccc(-c3ccc(N(c4ccc5ccc6cc7oc8ccccc8c7cc6c5c4)c4ccc5oc6ccccc6c5c4)cc3)c2)cc1 ; c1ccc(N(c2ccc3ccc4cc5oc6ccccc6c5cc4c3c2)c2cccc3oc4ccccc4c23)cc1 ; c1ccc(-c2ccc(N(c3ccc4ccc5cc6oc7ccccc7c6cc5c4c3)c3cccc4oc5ccccc5c34)cc2)cc1 ; c1ccc(-c2cccc(N(c3ccc4ccc5cc6oc7ccccc7c6cc5c4c3)c3cccc4oc5ccccc5c34)c2)cc1]

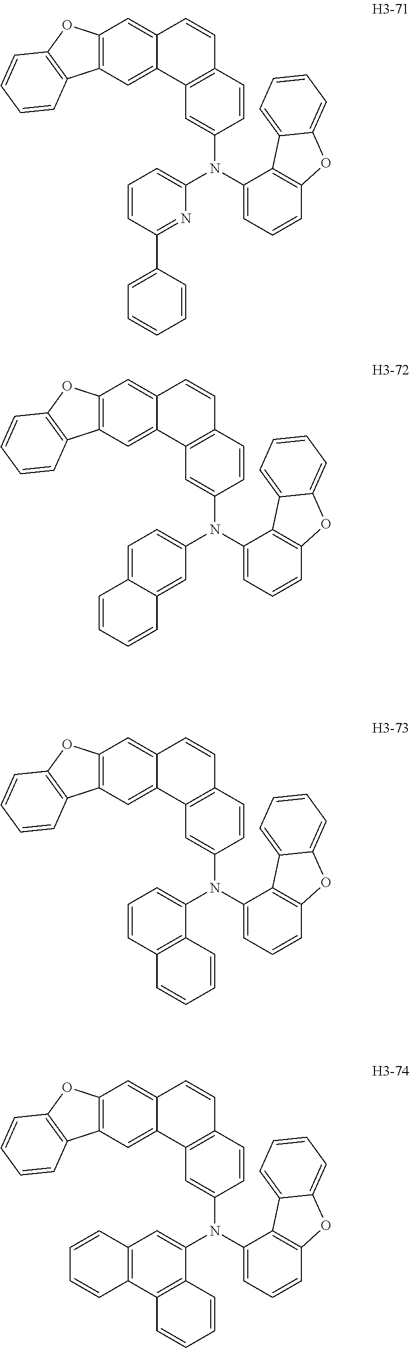 [SMILES: c1ccc(-c2cccc(N(c3ccc4ccc5cc6oc7ccccc7c6cc5c4c3)c3cccc4oc5ccccc5c34)n2)cc1 ; c1ccc2cc(N(c3ccc4ccc5cc6oc7ccccc7c6cc5c4c3)c3cccc4oc5ccccc5c34)ccc2c1 ; c1ccc2c(N(c3ccc4ccc5cc6oc7ccccc7c6cc5c4c3)c3cccc4oc5ccccc5c34)cccc2c1 ; c1ccc2c(c1)cc(N(c1ccc3ccc4cc5oc6ccccc6c5cc4c3c1)c1cccc3oc4ccccc4c13)c1ccccc12]

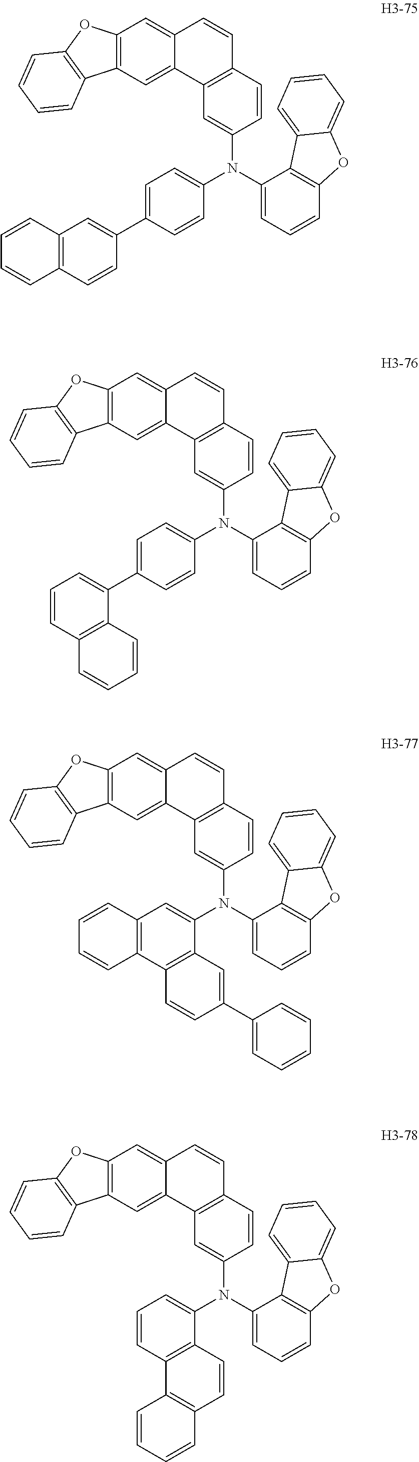 [SMILES: c1ccc2cc(-c3ccc(N(c4ccc5ccc6cc7oc8ccccc8c7cc6c5c4)c4cccc5oc6ccccc6c45)cc3)ccc2c1 ; c1ccc2c(-c3ccc(N(c4ccc5ccc6cc7oc8ccccc8c7cc6c5c4)c4cccc5oc6ccccc6c45)cc3)cccc2c1 ; c1ccc(-c2ccc3c(c2)c(N(c2ccc4ccc5cc6oc7ccccc7c6cc5c4c2)c2cccc4oc5ccccc5c24)cc2ccccc23)cc1 ; c1ccc2c(c1)ccc1c(N(c3ccc4ccc5cc6oc7ccccc7c6cc5c4c3)c3cccc4oc5ccccc5c34)cccc12]

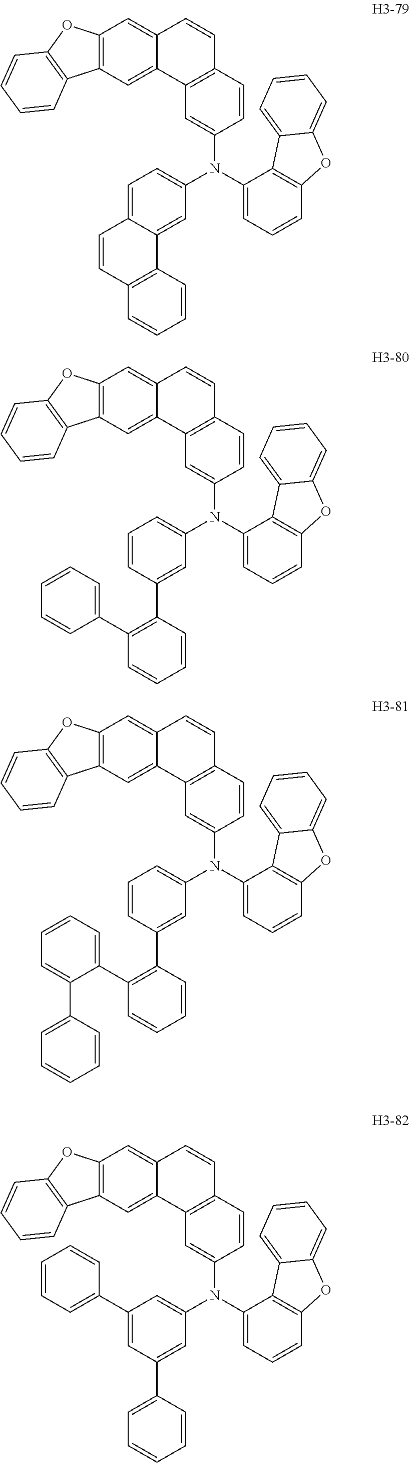

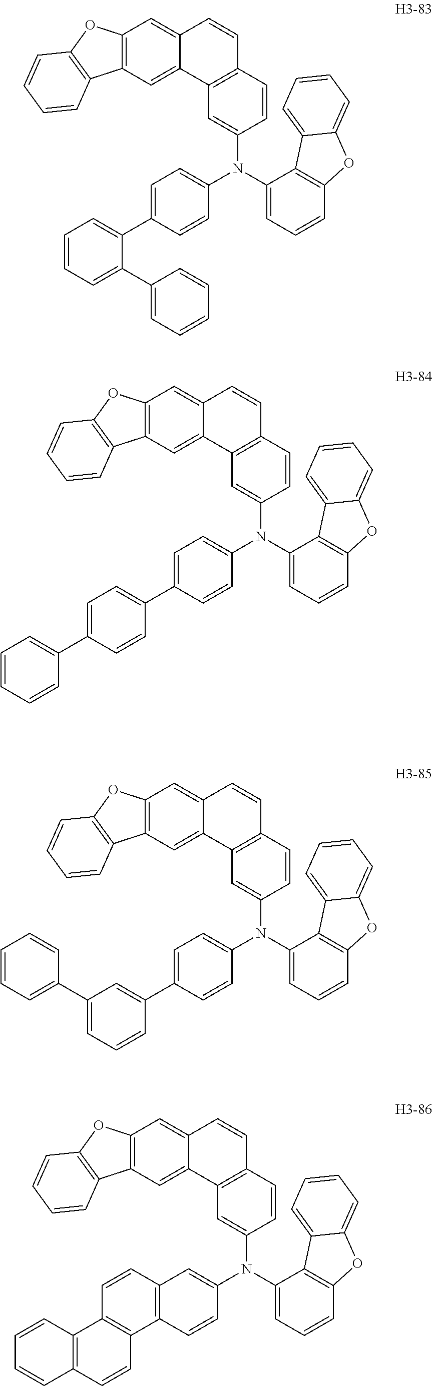 [SMILES: c1ccc(-c2ccccc2-c2ccc(N(c3ccc4ccc5cc6oc7ccccc7c6cc5c4c3)c3cccc4oc5ccccc5c34)cc2)cc1 ; c1ccc(-c2ccc(-c3ccc(N(c4ccc5ccc6cc7oc8ccccc8c7cc6c5c4)c4cccc5oc6ccccc6c45)cc3)cc2)cc1 ; c1ccc(-c2cccc(-c3ccc(N(c4ccc5ccc6cc7oc8ccccc8c7cc6c5c4)c4cccc5oc6ccccc6c45)cc3)c2)cc1 ; c1ccc2c(c1)ccc1c3ccc(N(c4ccc5ccc6cc7oc8ccccc8c7cc6c5c4)c4cccc5oc6ccccc6c45)cc3ccc21]

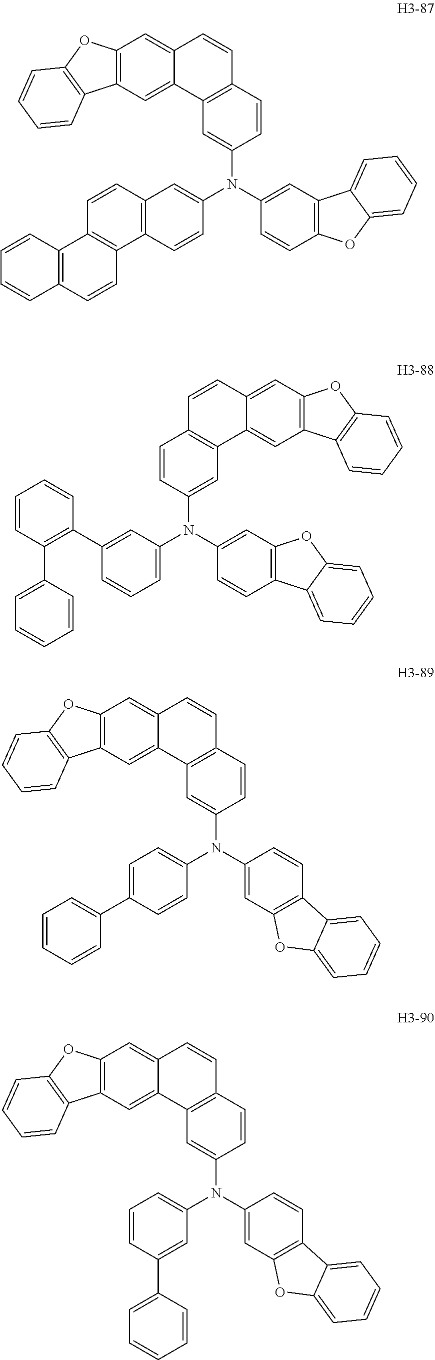 [SMILES: c1ccc2c(c1)ccc1c3ccc(N(c4ccc5ccc6cc7oc8ccccc8c7cc6c5c4)c4ccc5oc6ccccc6c5c4)cc3ccc21 ; c1ccc(-c2ccccc2-c2cccc(N(c3ccc4c(c3)oc3ccccc34)c3ccc4ccc5cc6oc7ccccc7c6cc5c4c3)c2)cc1 ; c1ccc(-c2ccc(N(c3ccc4c(c3)oc3ccccc34)c3ccc4ccc5cc6oc7ccccc7c6cc5c4c3)cc2)cc1 ; c1ccc(-c2cccc(N(c3ccc4c(c3)oc3ccccc34)c3ccc4ccc5cc6oc7ccccc7c6cc5c4c3)c2)cc1]

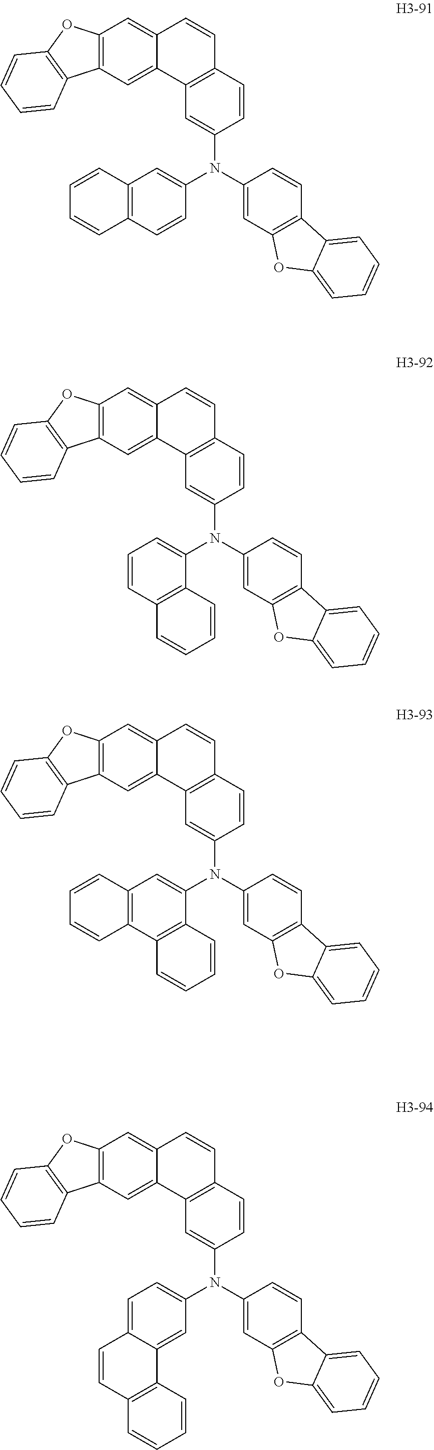 [SMILES: c1ccc2cc(N(c3ccc4c(c3)oc3ccccc34)c3ccc4ccc5cc6oc7ccccc7c6cc5c4c3)ccc2c1 ; c1ccc2c(N(c3ccc4c(c3)oc3ccccc34)c3ccc4ccc5cc6oc7ccccc7c6cc5c4c3)cccc2c1 ; c1ccc2c(c1)cc(N(c1ccc3c(c1)oc1ccccc13)c1ccc3ccc4cc5oc6ccccc6c5cc4c3c1)c1ccccc12 ; c1ccc2c(c1)ccc1ccc(N(c3ccc4c(c3)oc3ccccc34)c3ccc4ccc5cc6oc7ccccc7c6cc5c4c3)cc12]

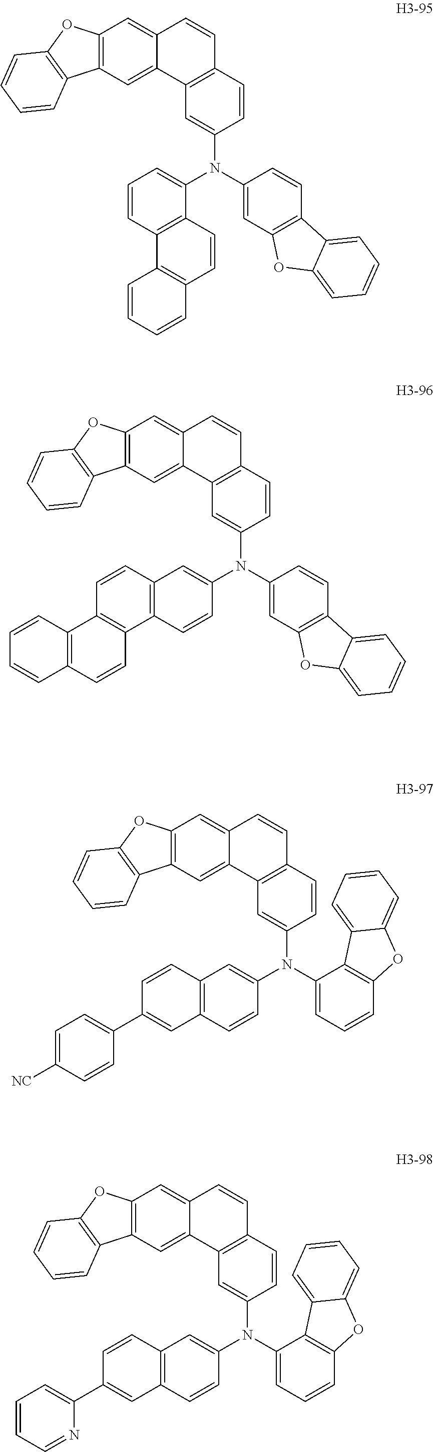 [SMILES: c1ccc2c(c1)ccc1c(N(c3ccc4c(c3)oc3ccccc34)c3ccc4ccc5cc6oc7ccccc7c6cc5c4c3)cccc12 ; c1ccc2c(c1)ccc1c3ccc(N(c4ccc5c(c4)oc4ccccc45)c4ccc5ccc6cc7oc8ccccc8c7cc6c5c4)cc3ccc21 ; N#Cc1ccc(-c2ccc3cc(N(c4ccc5ccc6cc7oc8ccccc8c7cc6c5c4)c4cccc5oc6ccccc6c45)ccc3c2)cc1 ; c1ccc(-c2ccc3cc(N(c4ccc5ccc6cc7oc8ccccc8c7cc6c5c4)c4cccc5oc6ccccc6c45)ccc3c2)nc1]

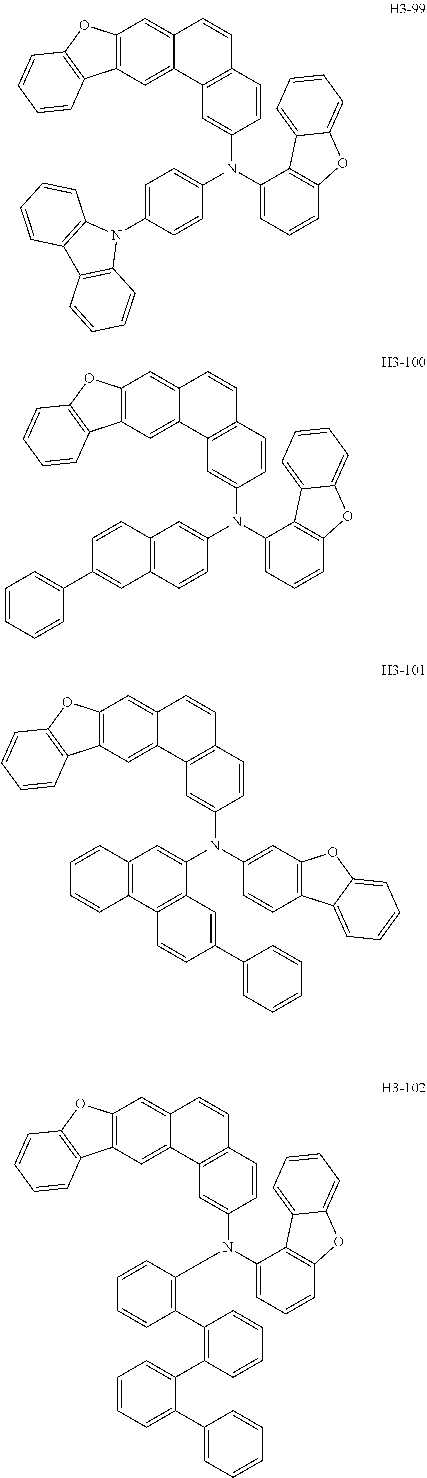

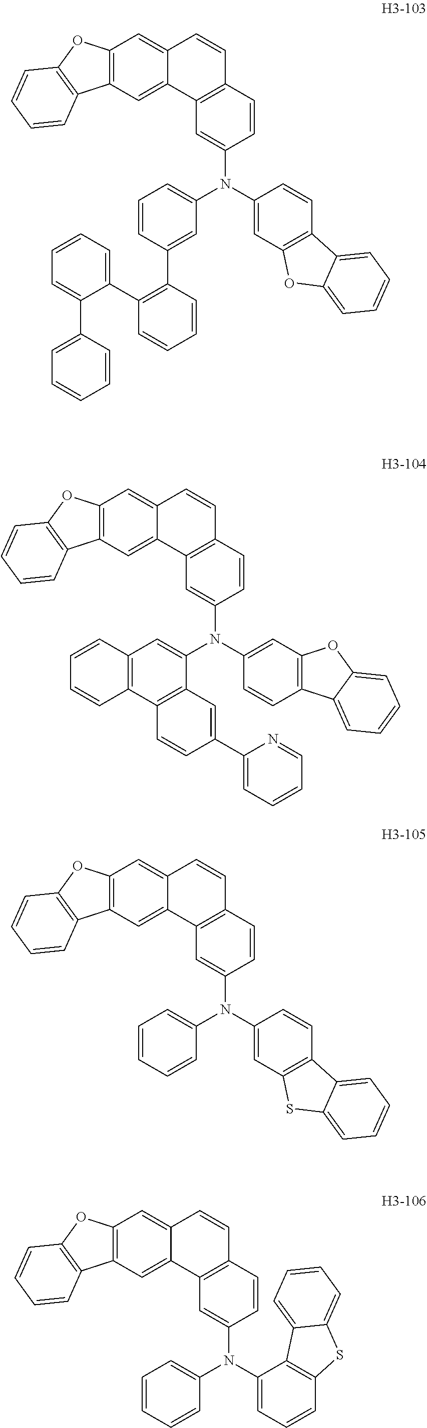 [SMILES: c1ccc(-c2ccccc2-c2ccccc2-c2cccc(N(c3ccc4c(c3)oc3ccccc34)c3ccc4ccc5cc6oc7ccccc7c6cc5c4c3)c2)cc1 ; c1ccc(-c2ccc3c(c2)c(N(c2ccc4c(c2)oc2ccccc24)c2ccc4ccc5cc6oc7ccccc7c6cc5c4c2)cc2ccccc23)nc1 ; c1ccc(N(c2ccc3c(c2)sc2ccccc23)c2ccc3ccc4cc5oc6ccccc6c5cc4c3c2)cc1 ; c1ccc(N(c2ccc3ccc4cc5oc6ccccc6c5cc4c3c2)c2cccc3sc4ccccc4c23)cc1]

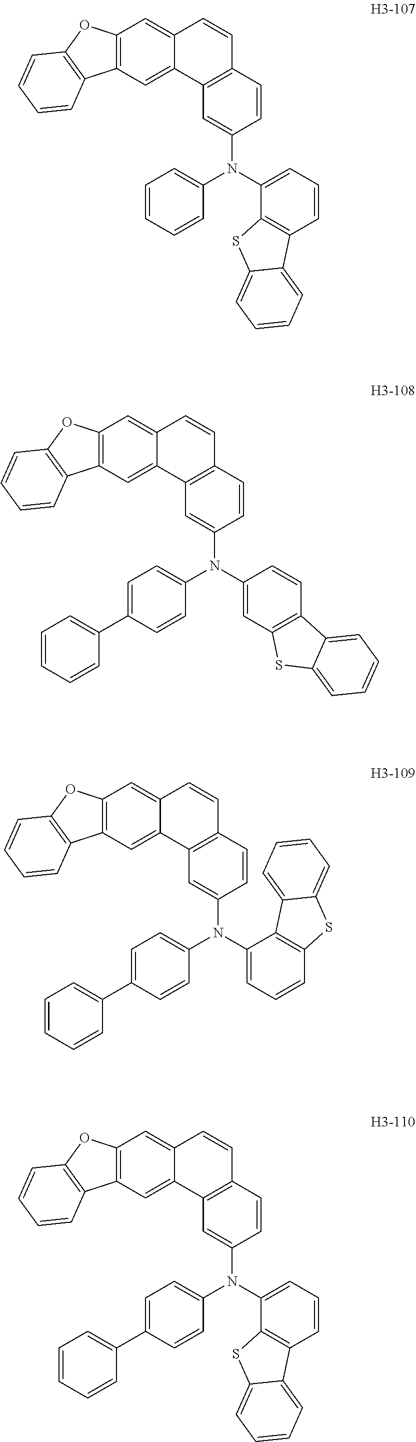 [SMILES: c1ccc(N(c2ccc3ccc4cc5oc6ccccc6c5cc4c3c2)c2cccc3c2sc2ccccc23)cc1 ; c1ccc(-c2ccc(N(c3ccc4c(c3)sc3ccccc34)c3ccc4ccc5cc6oc7ccccc7c6cc5c4c3)cc2)cc1 ; c1ccc(-c2ccc(N(c3ccc4ccc5cc6oc7ccccc7c6cc5c4c3)c3cccc4sc5ccccc5c34)cc2)cc1 ; c1ccc(-c2ccc(N(c3ccc4ccc5cc6oc7ccccc7c6cc5c4c3)c3cccc4c3sc3ccccc34)cc2)cc1]

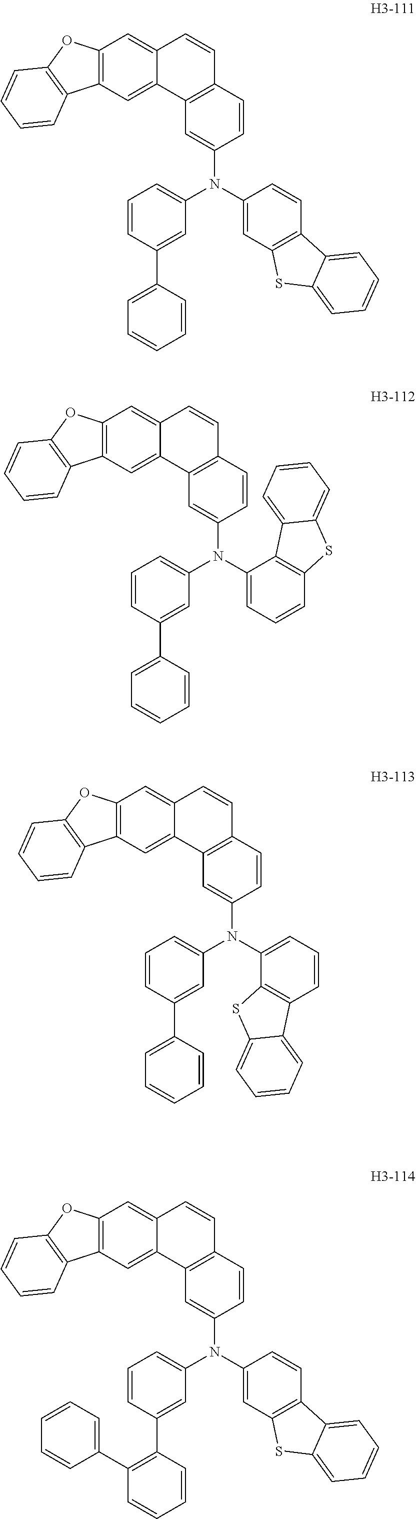 [SMILES: c1ccc(-c2cccc(N(c3ccc4c(c3)sc3ccccc34)c3ccc4ccc5cc6oc7ccccc7c6cc5c4c3)c2)cc1 ; c1ccc(-c2cccc(N(c3ccc4ccc5cc6oc7ccccc7c6cc5c4c3)c3cccc4sc5ccccc5c34)c2)cc1 ; c1ccc(-c2cccc(N(c3ccc4ccc5cc6oc7ccccc7c6cc5c4c3)c3cccc4c3sc3ccccc34)c2)cc1 ; c1ccc(-c2ccccc2-c2cccc(N(c3ccc4c(c3)sc3ccccc34)c3ccc4ccc5cc6oc7ccccc7c6cc5c4c3)c2)cc1]

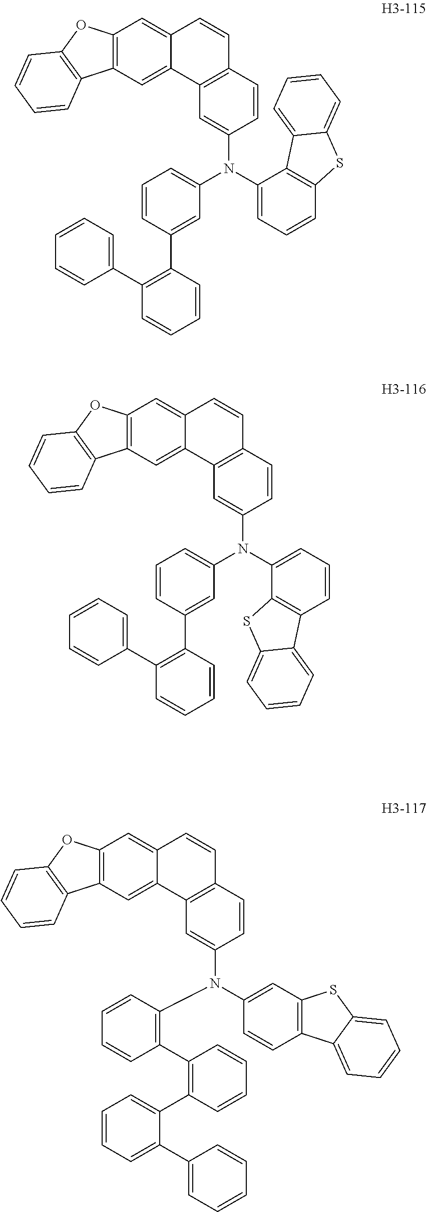 [SMILES: c1ccc(-c2ccccc2-c2cccc(N(c3ccc4ccc5cc6oc7ccccc7c6cc5c4c3)c3cccc4sc5ccccc5c34)c2)cc1 ; c1ccc(-c2ccccc2-c2cccc(N(c3ccc4ccc5cc6oc7ccccc7c6cc5c4c3)c3cccc4c3sc3ccccc34)c2)cc1 ; c1ccc(-c2ccccc2-c2ccccc2-c2ccccc2N(c2ccc3c(c2)sc2ccccc23)c2ccc3ccc4cc5oc6ccccc6c5cc4c3c2)cc1]

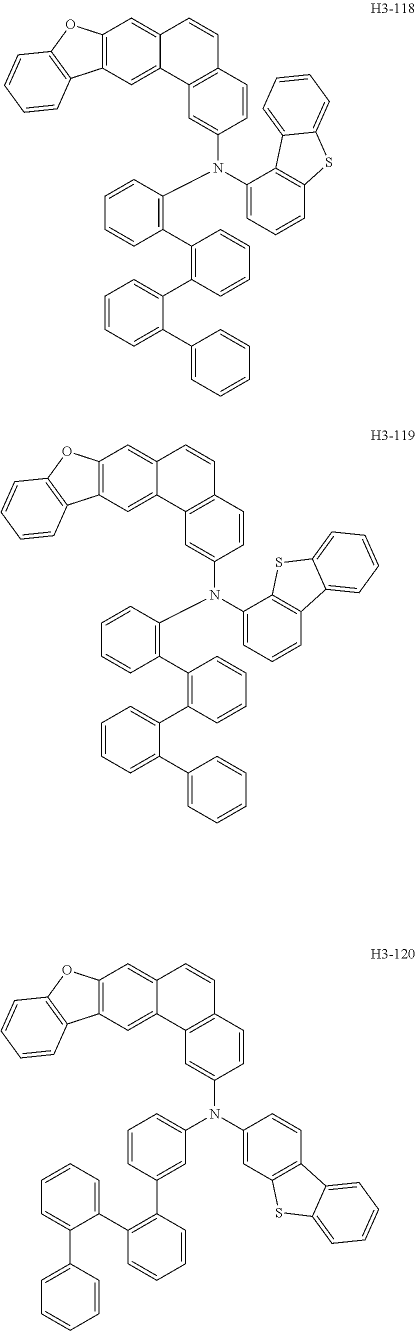 [SMILES: c1ccc(-c2ccccc2-c2ccccc2-c2ccccc2N(c2ccc3ccc4cc5oc6ccccc6c5cc4c3c2)c2cccc3sc4ccccc4c23)cc1 ; c1ccc(-c2ccccc2-c2ccccc2-c2ccccc2N(c2ccc3ccc4cc5oc6ccccc6c5cc4c3c2)c2cccc3c2sc2ccccc23)cc1 ; c1ccc(-c2ccccc2-c2ccccc2-c2cccc(N(c3ccc4c(c3)sc3ccccc34)c3ccc4ccc5cc6oc7ccccc7c6cc5c4c3)c2)cc1]

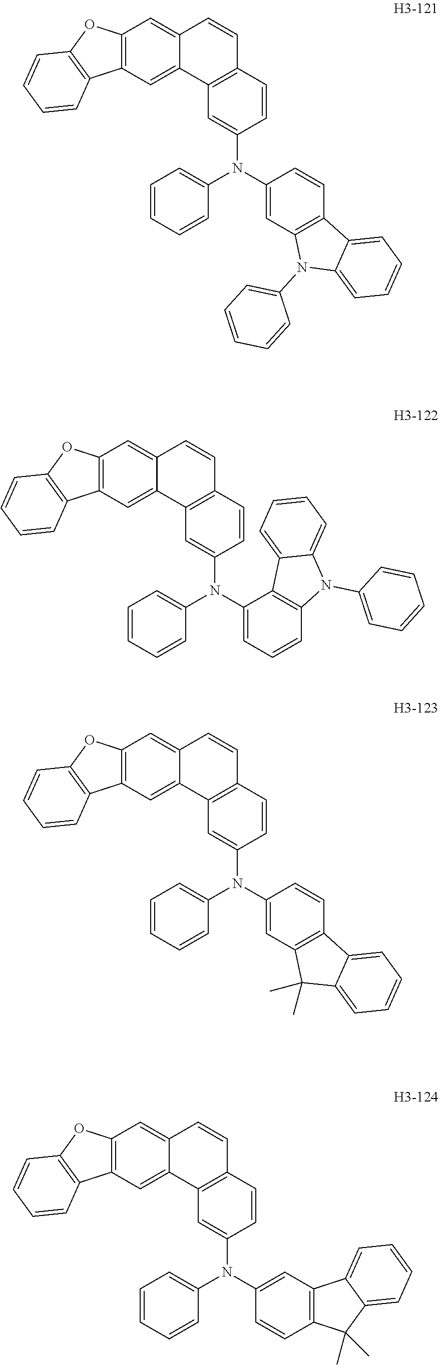 [SMILES: c1ccc(N(c2ccc3ccc4cc5oc6ccccc6c5cc4c3c2)c2ccc3c4ccccc4n(-c4ccccc4)c3c2)cc1 ; c1ccc(N(c2ccc3ccc4cc5oc6ccccc6c5cc4c3c2)c2cccc3c2c2ccccc2n3-c2ccccc2)cc1 ; CC1(C)c2ccccc2-c2ccc(N(c3ccccc3)c3ccc4ccc5cc6oc7ccccc7c6cc5c4c3)cc21 ; CC1(C)c2ccccc2-c2cc(N(c3ccccc3)c3ccc4ccc5cc6oc7ccccc7c6cc5c4c3)ccc21]

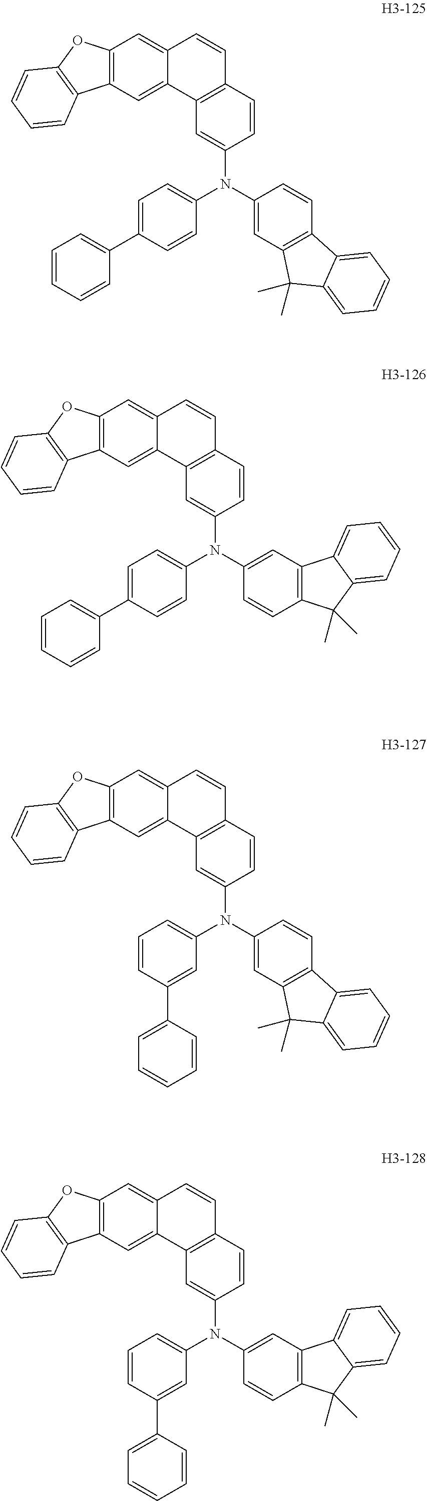 [SMILES: CC1(C)c2ccccc2-c2ccc(N(c3ccc(-c4ccccc4)cc3)c3ccc4ccc5cc6oc7ccccc7c6cc5c4c3)cc21 ; CC1(C)c2ccccc2-c2cc(N(c3ccc(-c4ccccc4)cc3)c3ccc4ccc5cc6oc7ccccc7c6cc5c4c3)ccc21 ; CC1(C)c2ccccc2-c2ccc(N(c3cccc(-c4ccccc4)c3)c3ccc4ccc5cc6oc7ccccc7c6cc5c4c3)cc21 ; CC1(C)c2ccccc2-c2cc(N(c3cccc(-c4ccccc4)c3)c3ccc4ccc5cc6oc7ccccc7c6cc5c4c3)ccc21]

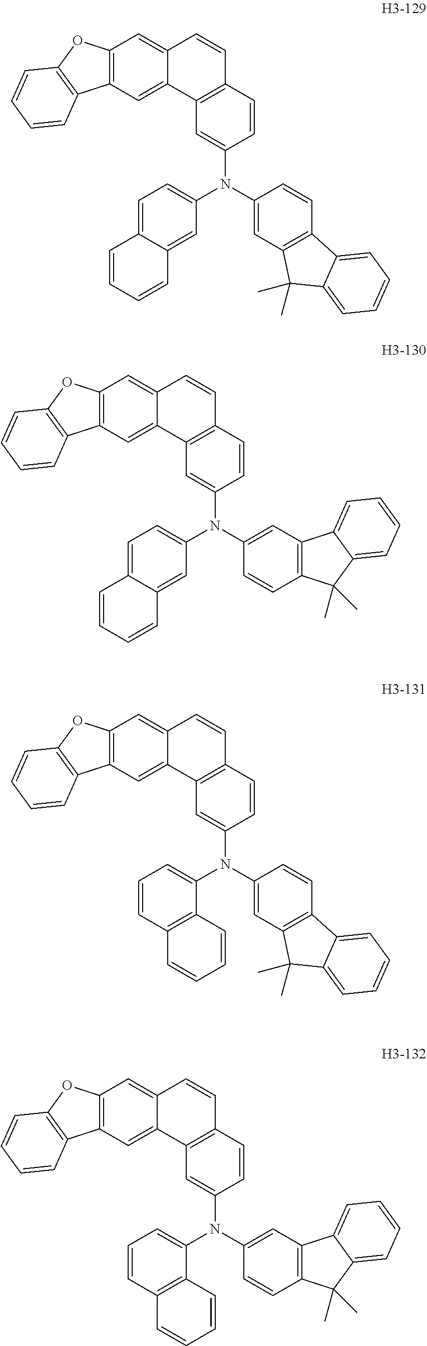 [SMILES: CC1(C)c2ccccc2-c2ccc(N(c3ccc4ccccc4c3)c3ccc4ccc5cc6oc7ccccc7c6cc5c4c3)cc21 ; CC1(C)c2ccccc2-c2cc(N(c3ccc4ccccc4c3)c3ccc4ccc5cc6oc7ccccc7c6cc5c4c3)ccc21 ; CC1(C)c2ccccc2-c2ccc(N(c3ccc4ccc5cc6oc7ccccc7c6cc5c4c3)c3cccc4ccccc34)cc21 ; CC1(C)c2ccccc2-c2cc(N(c3ccc4ccc5cc6oc7ccccc7c6cc5c4c3)c3cccc4ccccc34)ccc21]

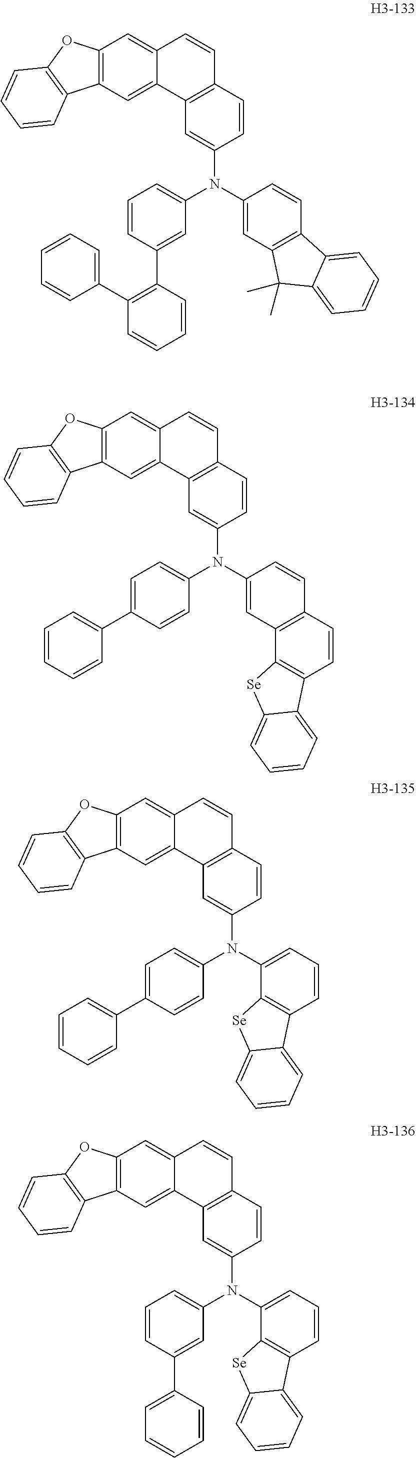 [SMILES: CC1(C)c2ccccc2-c2ccc(N(c3cccc(-c4ccccc4-c4ccccc4)c3)c3ccc4ccc5cc6oc7ccccc7c6cc5c4c3)cc21 ; c1ccc(-c2ccc(N(c3ccc4ccc5cc6oc7ccccc7c6cc5c4c3)c3ccc4ccc5c6ccccc6[se]c5c4c3)cc2)cc1 ; c1ccc(-c2ccc(N(c3ccc4ccc5cc6oc7ccccc7c6cc5c4c3)c3cccc4c3[se]c3ccccc34)cc2)cc1 ; c1ccc(-c2cccc(N(c3ccc4ccc5cc6oc7ccccc7c6cc5c4c3)c3cccc4c3[se]c3ccccc34)c2)cc1]

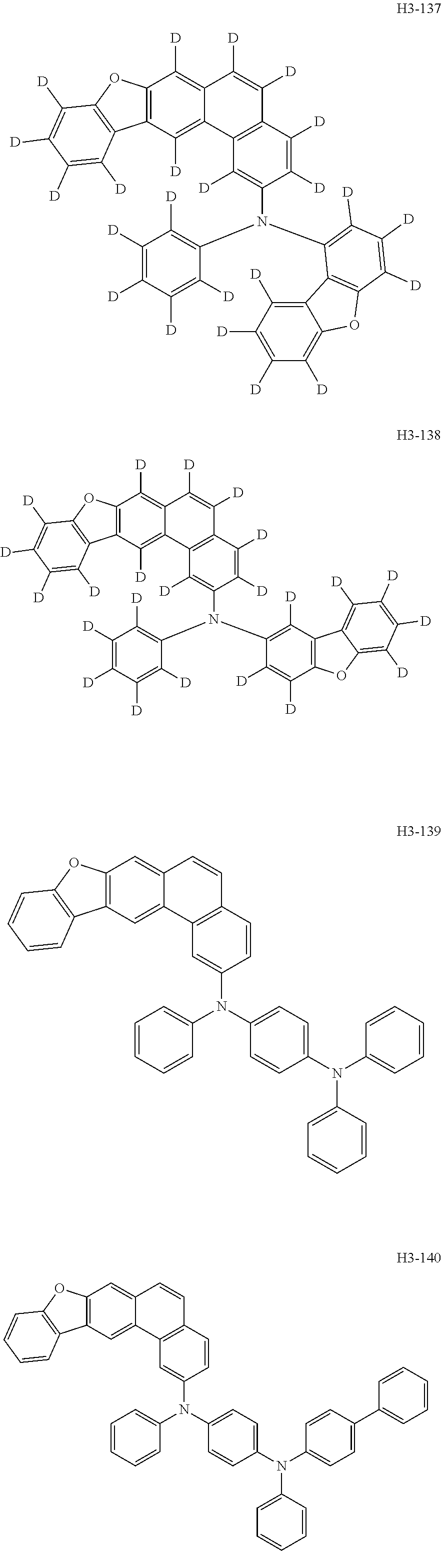

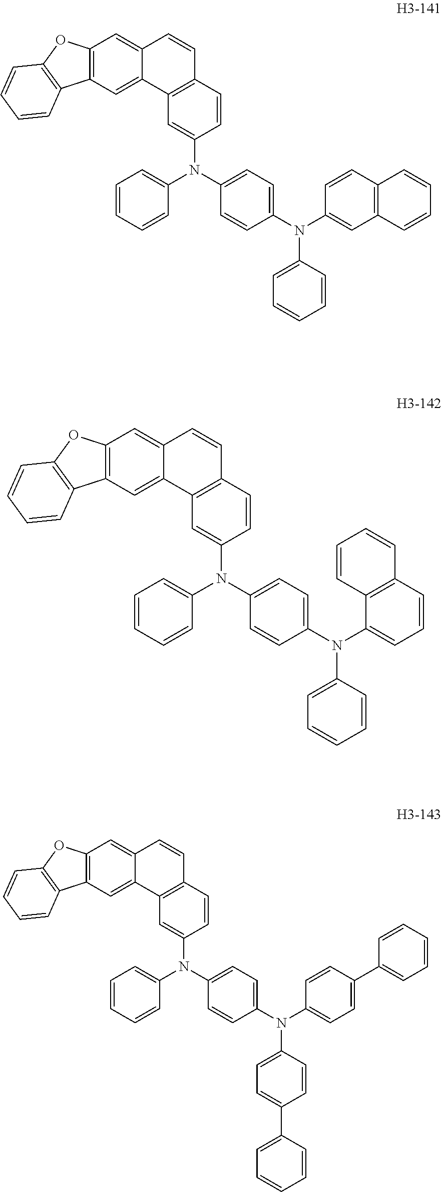 [SMILES: c1ccc(N(c2ccc(N(c3ccccc3)c3ccc4ccc5cc6oc7ccccc7c6cc5c4c3)cc2)c2ccc3ccccc3c2)cc1 ; c1ccc(N(c2ccc(N(c3ccccc3)c3cccc4ccccc34)cc2)c2ccc3ccc4cc5oc6ccccc6c5cc4c3c2)cc1 ; c1ccc(-c2ccc(N(c3ccc(-c4ccccc4)cc3)c3ccc(N(c4ccccc4)c4ccc5ccc6cc7oc8ccccc8c7cc6c5c4)cc3)cc2)cc1]

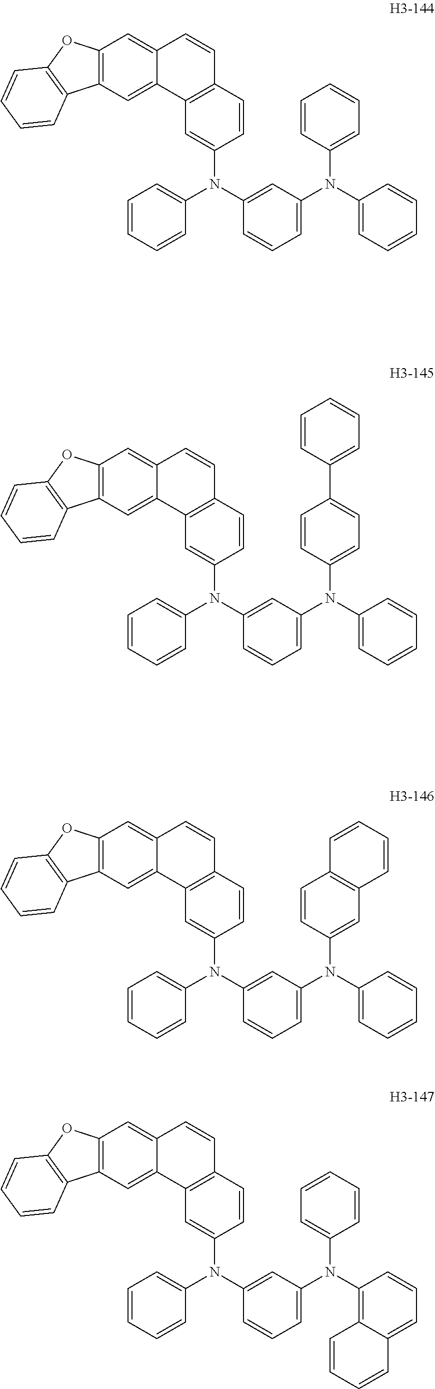 [SMILES: c1ccc(N(c2ccccc2)c2cccc(N(c3ccccc3)c3ccc4ccc5cc6oc7ccccc7c6cc5c4c3)c2)cc1 ; c1ccc(-c2ccc(N(c3ccccc3)c3cccc(N(c4ccccc4)c4ccc5ccc6cc7oc8ccccc8c7cc6c5c4)c3)cc2)cc1 ; c1ccc(N(c2cccc(N(c3ccccc3)c3ccc4ccc5cc6oc7ccccc7c6cc5c4c3)c2)c2ccc3ccccc3c2)cc1 ; c1ccc(N(c2cccc(N(c3ccccc3)c3cccc4ccccc34)c2)c2ccc3ccc4cc5oc6ccccc6c5cc4c3c2)cc1]

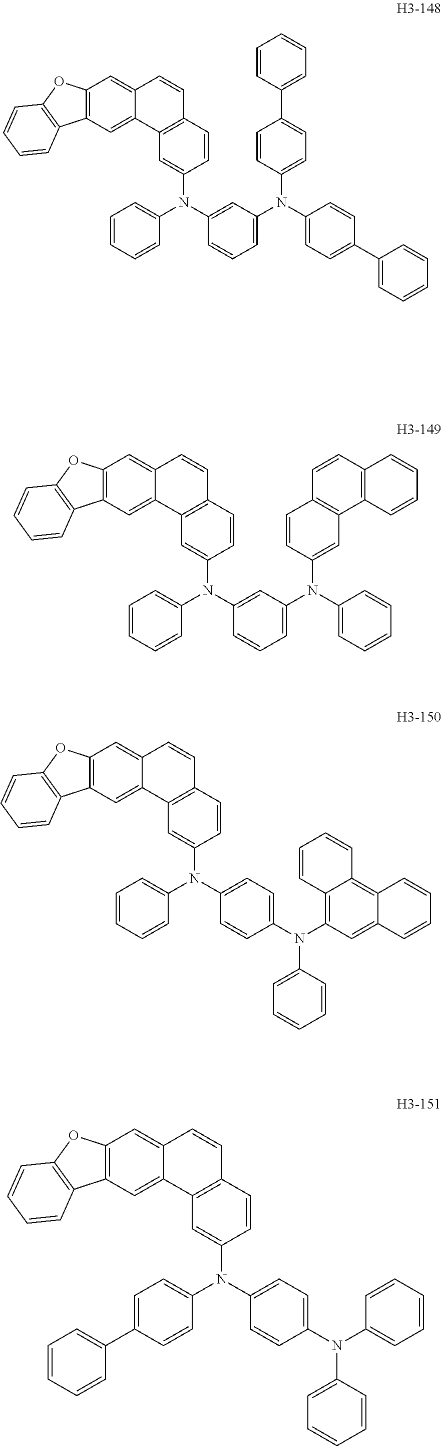 [SMILES: c1ccc(-c2ccc(N(c3ccc(-c4ccccc4)cc3)c3cccc(N(c4ccccc4)c4ccc5ccc6cc7oc8ccccc8c7cc6c5c4)c3)cc2)cc1 ; c1ccc(N(c2cccc(N(c3ccccc3)c3ccc4ccc5cc6oc7ccccc7c6cc5c4c3)c2)c2ccc3ccc4ccccc4c3c2)cc1 ; c1ccc(N(c2ccc(N(c3ccccc3)c3cc4ccccc4c4ccccc34)cc2)c2ccc3ccc4cc5oc6ccccc6c5cc4c3c2)cc1 ; c1ccc(-c2ccc(N(c3ccc(N(c4ccccc4)c4ccccc4)cc3)c3ccc4ccc5cc6oc7ccccc7c6cc5c4c3)cc2)cc1]

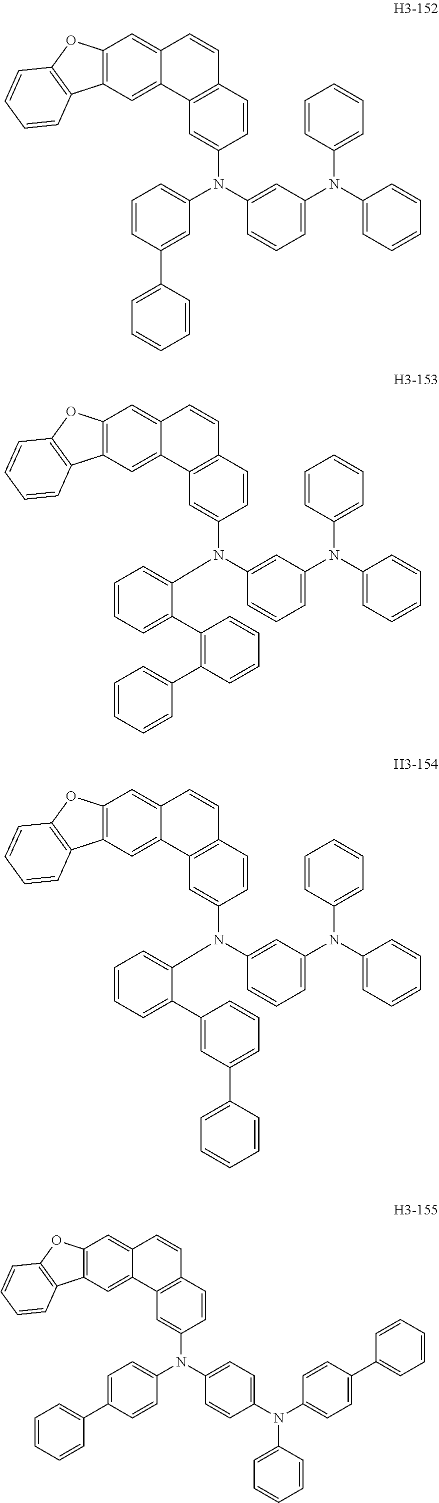 [SMILES: c1ccc(-c2cccc(N(c3cccc(N(c4ccccc4)c4ccccc4)c3)c3ccc4ccc5cc6oc7ccccc7c6cc5c4c3)c2)cc1 ; c1ccc(-c2ccccc2-c2ccccc2N(c2cccc(N(c3ccccc3)c3ccccc3)c2)c2ccc3ccc4cc5oc6ccccc6c5cc4c3c2)cc1 ; c1ccc(-c2cccc(-c3ccccc3N(c3cccc(N(c4ccccc4)c4ccccc4)c3)c3ccc4ccc5cc6oc7ccccc7c6cc5c4c3)c2)cc1 ; c1ccc(-c2ccc(N(c3ccccc3)c3ccc(N(c4ccc(-c5ccccc5)cc4)c4ccc5ccc6cc7oc8ccccc8c7cc6c5c4)cc3)cc2)cc1]

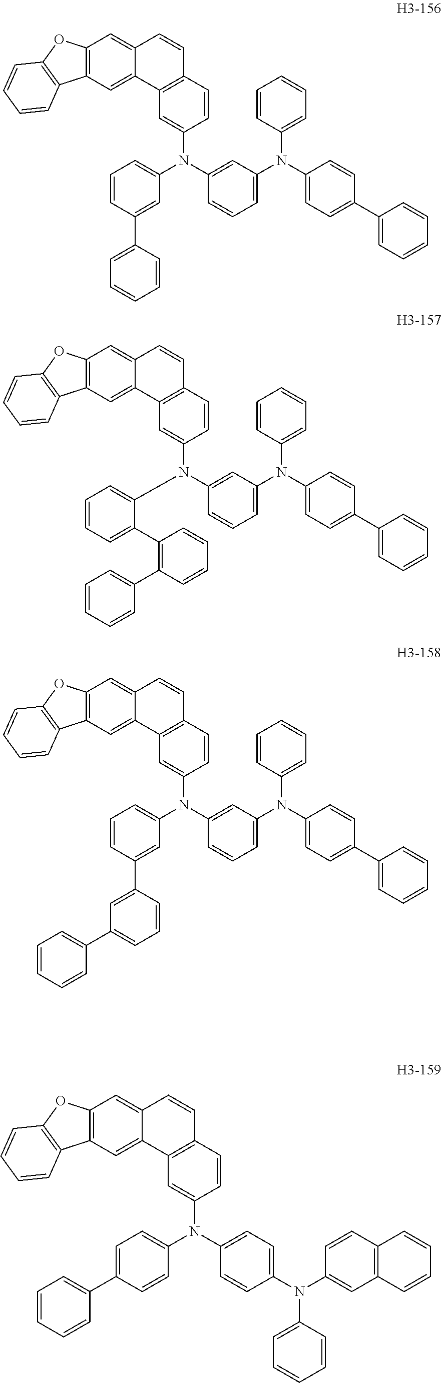 [SMILES: c1ccc(-c2ccc(N(c3ccccc3)c3cccc(N(c4cccc(-c5ccccc5)c4)c4ccc5ccc6cc7oc8ccccc8c7cc6c5c4)c3)cc2)cc1 ; c1ccc(-c2ccc(N(c3ccccc3)c3cccc(N(c4ccc5ccc6cc7oc8ccccc8c7cc6c5c4)c4ccccc4-c4ccccc4-c4ccccc4)c3)cc2)cc1 ; c1ccc(-c2ccc(N(c3ccccc3)c3cccc(N(c4cccc(-c5cccc(-c6ccccc6)c5)c4)c4ccc5ccc6cc7oc8ccccc8c7cc6c5c4)c3)cc2)cc1 ; c1ccc(-c2ccc(N(c3ccc(N(c4ccccc4)c4ccc5ccccc5c4)cc3)c3ccc4ccc5cc6oc7ccccc7c6cc5c4c3)cc2)cc1]

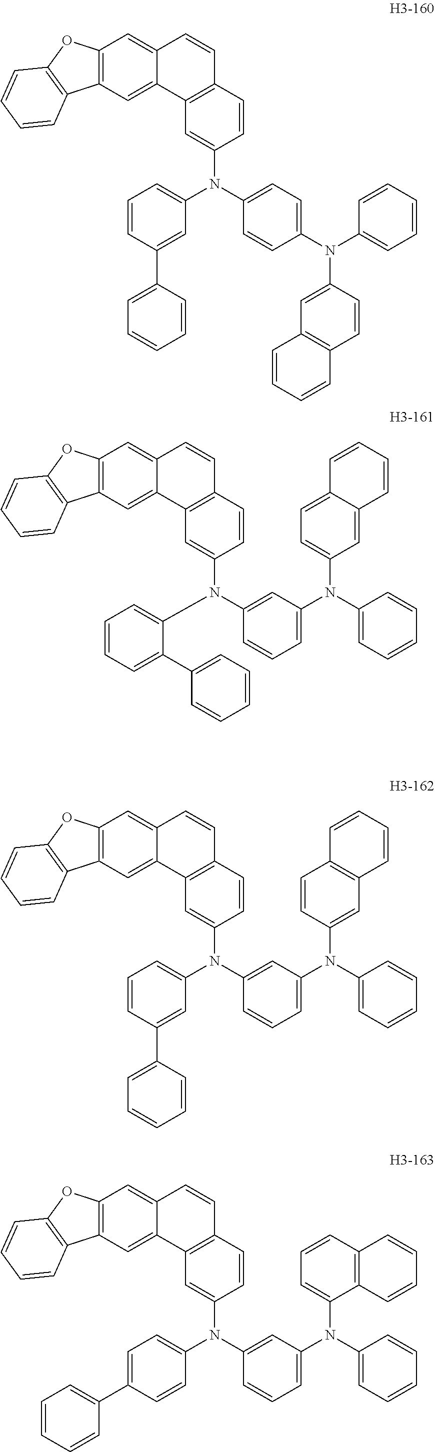 [SMILES: c1ccc(-c2cccc(N(c3ccc(N(c4ccccc4)c4ccc5ccccc5c4)cc3)c3ccc4ccc5cc6oc7ccccc7c6cc5c4c3)c2)cc1 ; c1ccc(-c2ccccc2N(c2cccc(N(c3ccccc3)c3ccc4ccccc4c3)c2)c2ccc3ccc4cc5oc6ccccc6c5cc4c3c2)cc1 ; c1ccc(-c2cccc(N(c3cccc(N(c4ccccc4)c4ccc5ccccc5c4)c3)c3ccc4ccc5cc6oc7ccccc7c6cc5c4c3)c2)cc1 ; c1ccc(-c2ccc(N(c3cccc(N(c4ccccc4)c4cccc5ccccc45)c3)c3ccc4ccc5cc6oc7ccccc7c6cc5c4c3)cc2)cc1]

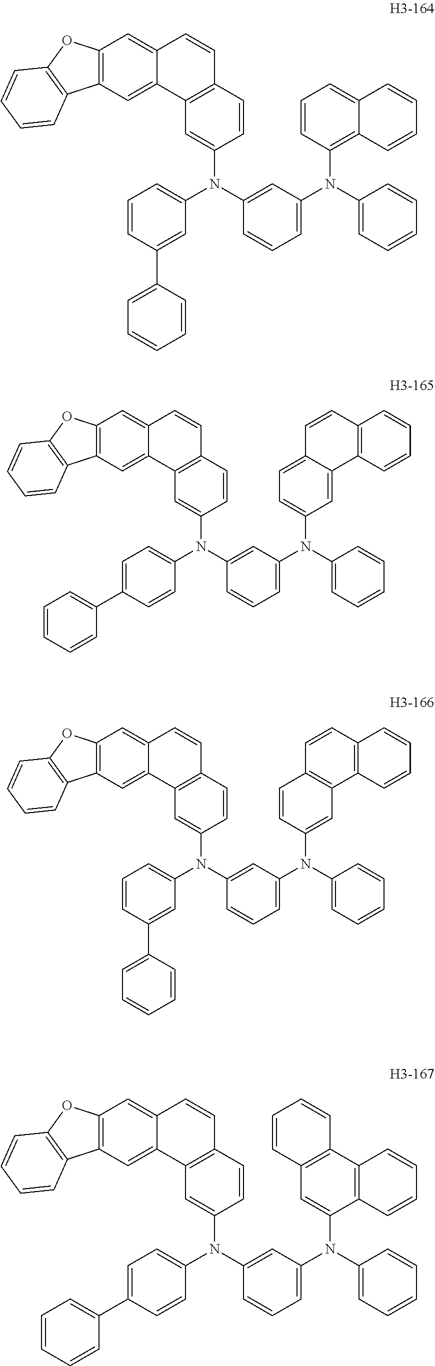 [SMILES: c1ccc(-c2cccc(N(c3cccc(N(c4ccccc4)c4cccc5ccccc45)c3)c3ccc4ccc5cc6oc7ccccc7c6cc5c4c3)c2)cc1 ; c1ccc(-c2ccc(N(c3cccc(N(c4ccccc4)c4ccc5ccc6ccccc6c5c4)c3)c3ccc4ccc5cc6oc7ccccc7c6cc5c4c3)cc2)cc1 ; c1ccc(-c2cccc(N(c3cccc(N(c4ccccc4)c4ccc5ccc6ccccc6c5c4)c3)c3ccc4ccc5cc6oc7ccccc7c6cc5c4c3)c2)cc1 ; c1ccc(-c2ccc(N(c3cccc(N(c4ccccc4)c4cc5ccccc5c5ccccc45)c3)c3ccc4ccc5cc6oc7ccccc7c6cc5c4c3)cc2)cc1]

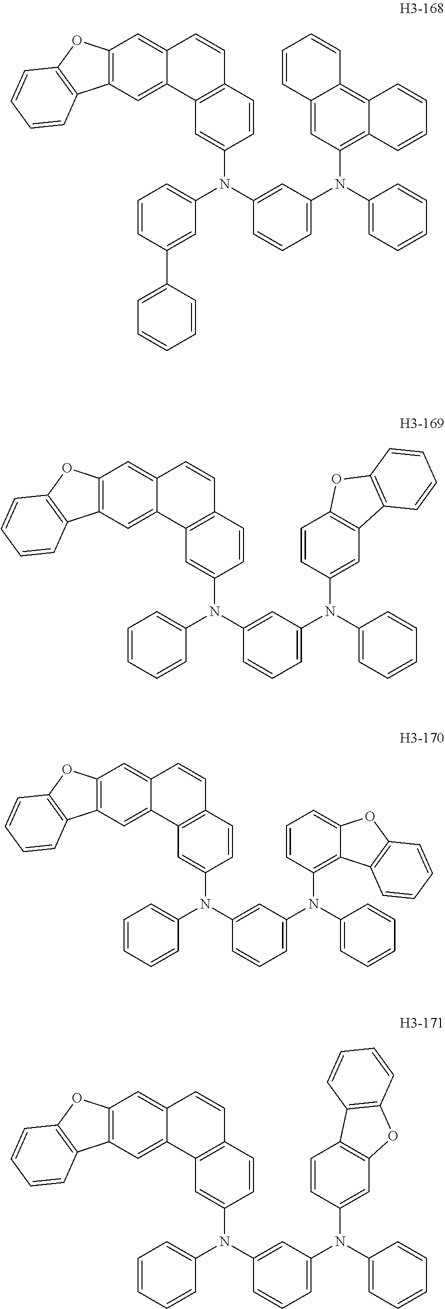 [SMILES: c1ccc(-c2cccc(N(c3cccc(N(c4ccccc4)c4cc5ccccc5c5ccccc45)c3)c3ccc4ccc5cc6oc7ccccc7c6cc5c4c3)c2)cc1 ; c1ccc(N(c2cccc(N(c3ccccc3)c3ccc4oc5ccccc5c4c3)c2)c2ccc3ccc4cc5oc6ccccc6c5cc4c3c2)cc1 ; c1ccc(N(c2cccc(N(c3ccccc3)c3cccc4oc5ccccc5c34)c2)c2ccc3ccc4cc5oc6ccccc6c5cc4c3c2)cc1 ; c1ccc(N(c2cccc(N(c3ccccc3)c3ccc4ccc5cc6oc7ccccc7c6cc5c4c3)c2)c2ccc3c(c2)oc2ccccc23)cc1]

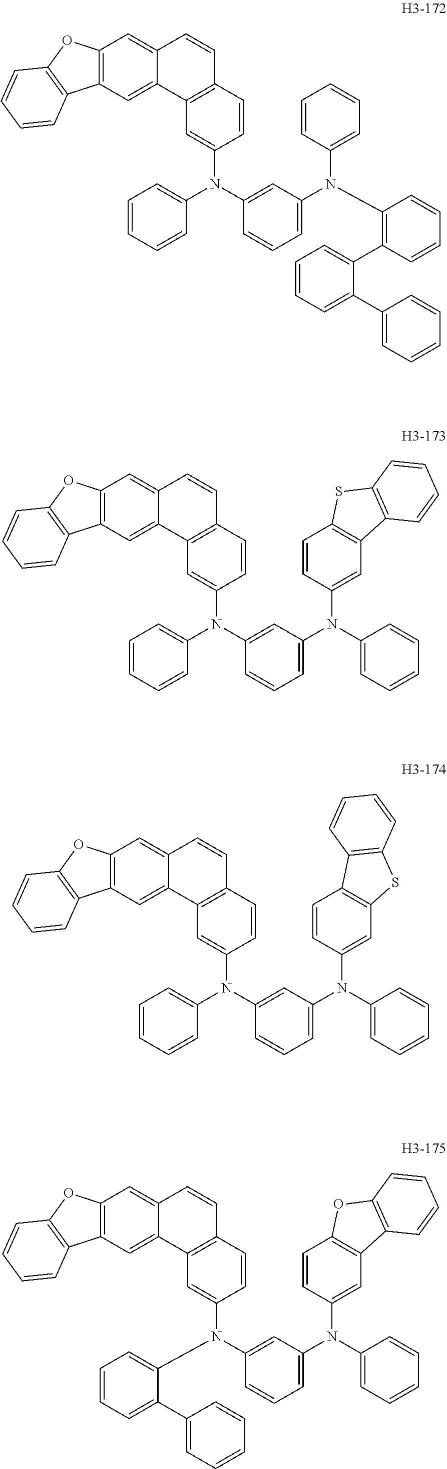 [SMILES: c1ccc(-c2ccccc2-c2ccccc2N(c2ccccc2)c2cccc(N(c3ccccc3)c3ccc4ccc5cc6oc7ccccc7c6cc5c4c3)c2)cc1 ; c1ccc(N(c2cccc(N(c3ccccc3)c3ccc4sc5ccccc5c4c3)c2)c2ccc3ccc4cc5oc6ccccc6c5cc4c3c2)cc1 ; c1ccc(N(c2cccc(N(c3ccccc3)c3ccc4ccc5cc6oc7ccccc7c6cc5c4c3)c2)c2ccc3c(c2)sc2ccccc23)cc1 ; c1ccc(-c2ccccc2N(c2cccc(N(c3ccccc3)c3ccc4oc5ccccc5c4c3)c2)c2ccc3ccc4cc5oc6ccccc6c5cc4c3c2)cc1]

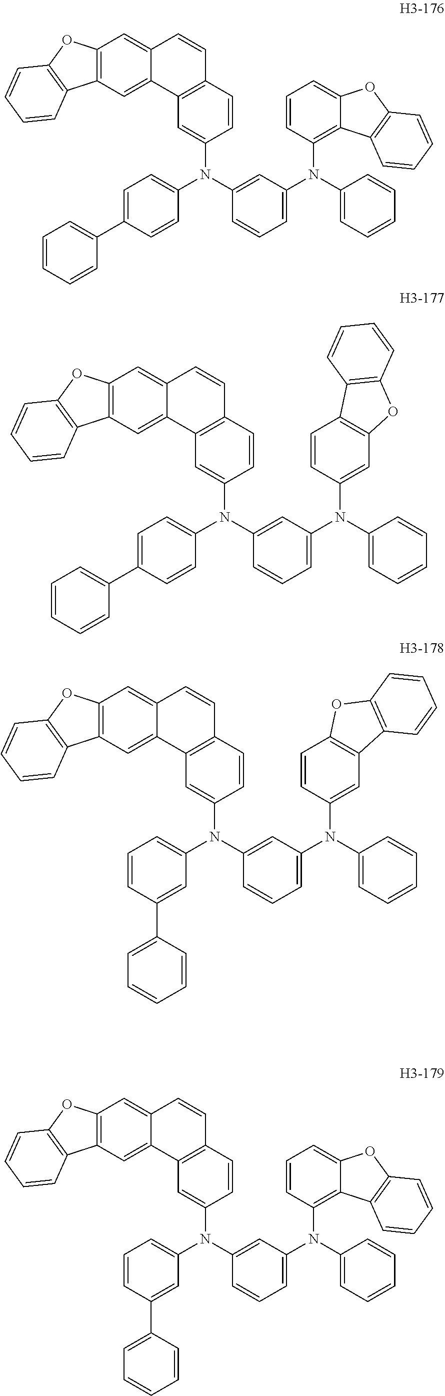 [SMILES: c1ccc(-c2ccc(N(c3cccc(N(c4ccccc4)c4cccc5oc6ccccc6c45)c3)c3ccc4ccc5cc6oc7ccccc7c6cc5c4c3)cc2)cc1 ; c1ccc(-c2ccc(N(c3cccc(N(c4ccccc4)c4ccc5c(c4)oc4ccccc45)c3)c3ccc4ccc5cc6oc7ccccc7c6cc5c4c3)cc2)cc1 ; c1ccc(-c2cccc(N(c3cccc(N(c4ccccc4)c4ccc5oc6ccccc6c5c4)c3)c3ccc4ccc5cc6oc7ccccc7c6cc5c4c3)c2)cc1 ; c1ccc(-c2cccc(N(c3cccc(N(c4ccccc4)c4cccc5oc6ccccc6c45)c3)c3ccc4ccc5cc6oc7ccccc7c6cc5c4c3)c2)cc1]

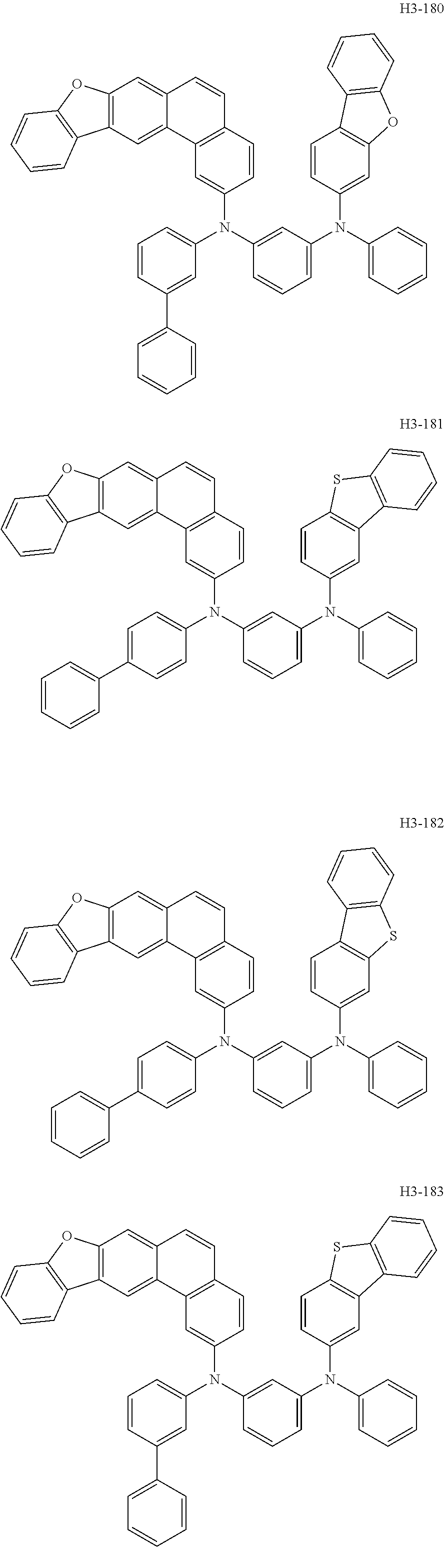 [SMILES: c1ccc(-c2cccc(N(c3cccc(N(c4ccccc4)c4ccc5c(c4)oc4ccccc45)c3)c3ccc4ccc5cc6oc7ccccc7c6cc5c4c3)c2)cc1 ; c1ccc(-c2ccc(N(c3cccc(N(c4ccccc4)c4ccc5sc6ccccc6c5c4)c3)c3ccc4ccc5cc6oc7ccccc7c6cc5c4c3)cc2)cc1 ; c1ccc(-c2ccc(N(c3cccc(N(c4ccccc4)c4ccc5c(c4)sc4ccccc45)c3)c3ccc4ccc5cc6oc7ccccc7c6cc5c4c3)cc2)cc1 ; c1ccc(-c2cccc(N(c3cccc(N(c4ccccc4)c4ccc5sc6ccccc6c5c4)c3)c3ccc4ccc5cc6oc7ccccc7c6cc5c4c3)c2)cc1]

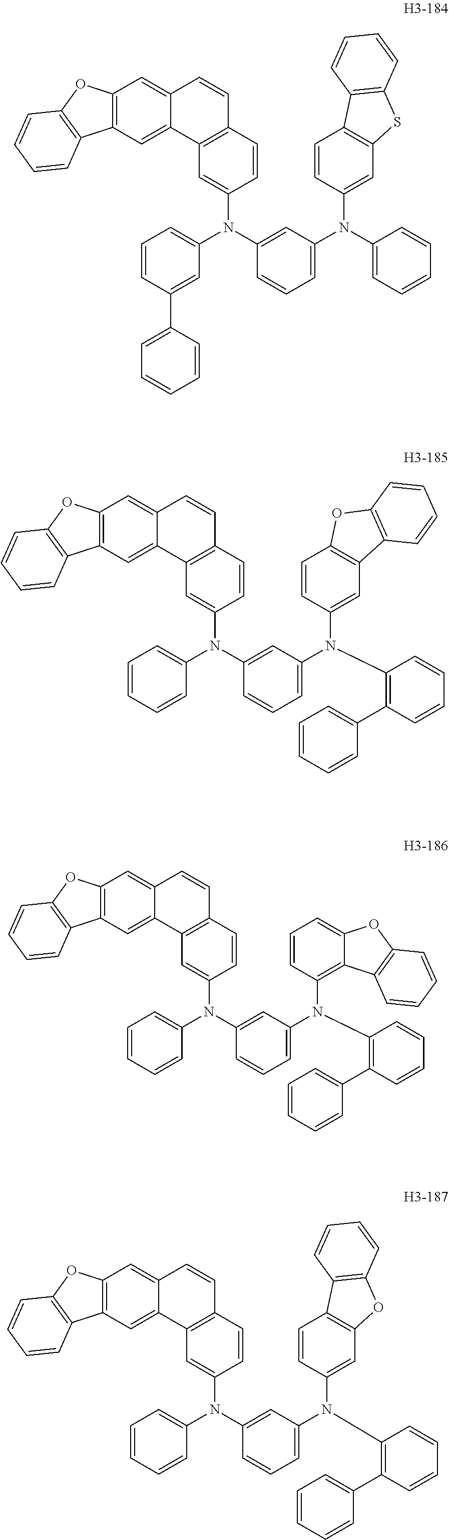 [SMILES: c1ccc(-c2cccc(N(c3cccc(N(c4ccccc4)c4ccc5c(c4)sc4ccccc45)c3)c3ccc4ccc5cc6oc7ccccc7c6cc5c4c3)c2)cc1 ; c1ccc(-c2ccccc2N(c2cccc(N(c3ccccc3)c3ccc4ccc5cc6oc7ccccc7c6cc5c4c3)c2)c2ccc3oc4ccccc4c3c2)cc1 ; c1ccc(-c2ccccc2N(c2cccc(N(c3ccccc3)c3ccc4ccc5cc6oc7ccccc7c6cc5c4c3)c2)c2cccc3oc4ccccc4c23)cc1 ; c1ccc(-c2ccccc2N(c2cccc(N(c3ccccc3)c3ccc4ccc5cc6oc7ccccc7c6cc5c4c3)c2)c2ccc3c(c2)oc2ccccc23)cc1]

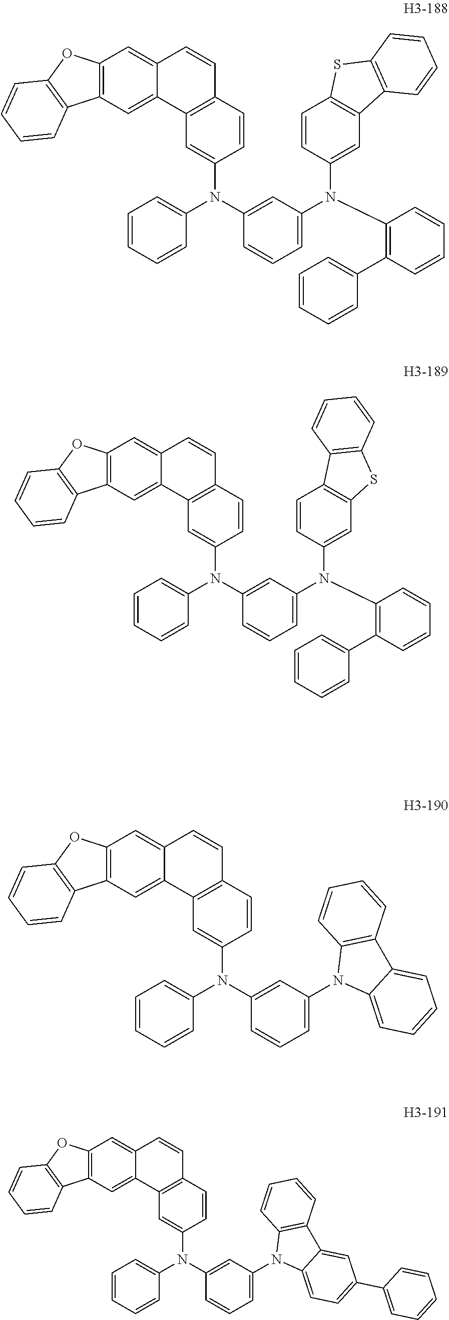 [SMILES: c1ccc(-c2ccccc2N(c2cccc(N(c3ccccc3)c3ccc4ccc5cc6oc7ccccc7c6cc5c4c3)c2)c2ccc3sc4ccccc4c3c2)cc1 ; c1ccc(-c2ccccc2N(c2cccc(N(c3ccccc3)c3ccc4ccc5cc6oc7ccccc7c6cc5c4c3)c2)c2ccc3c(c2)sc2ccccc23)cc1 ; c1ccc(N(c2cccc(-n3c4ccccc4c4ccccc43)c2)c2ccc3ccc4cc5oc6ccccc6c5cc4c3c2)cc1 ; c1ccc(-c2ccc3c(c2)c2ccccc2n3-c2cccc(N(c3ccccc3)c3ccc4ccc5cc6oc7ccccc7c6cc5c4c3)c2)cc1]

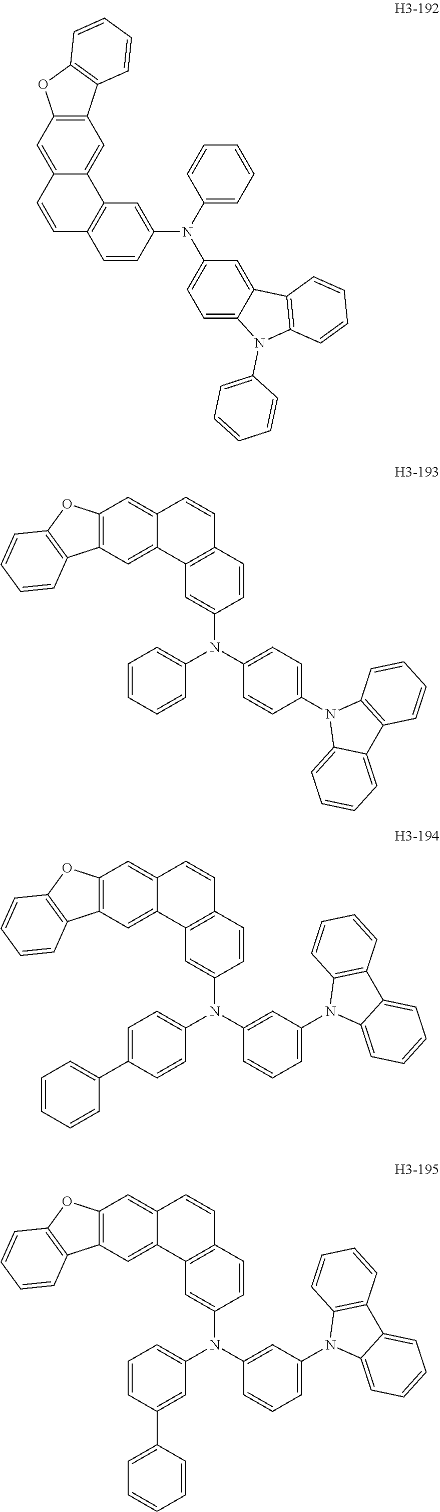 [SMILES: c1ccc(N(c2ccc3ccc4cc5oc6ccccc6c5cc4c3c2)c2ccc3c(c2)c2ccccc2n3-c2ccccc2)cc1 ; c1ccc(N(c2ccc(-n3c4ccccc4c4ccccc43)cc2)c2ccc3ccc4cc5oc6ccccc6c5cc4c3c2)cc1 ; c1ccc(-c2ccc(N(c3cccc(-n4c5ccccc5c5ccccc54)c3)c3ccc4ccc5cc6oc7ccccc7c6cc5c4c3)cc2)cc1 ; c1ccc(-c2cccc(N(c3cccc(-n4c5ccccc5c5ccccc54)c3)c3ccc4ccc5cc6oc7ccccc7c6cc5c4c3)c2)cc1]

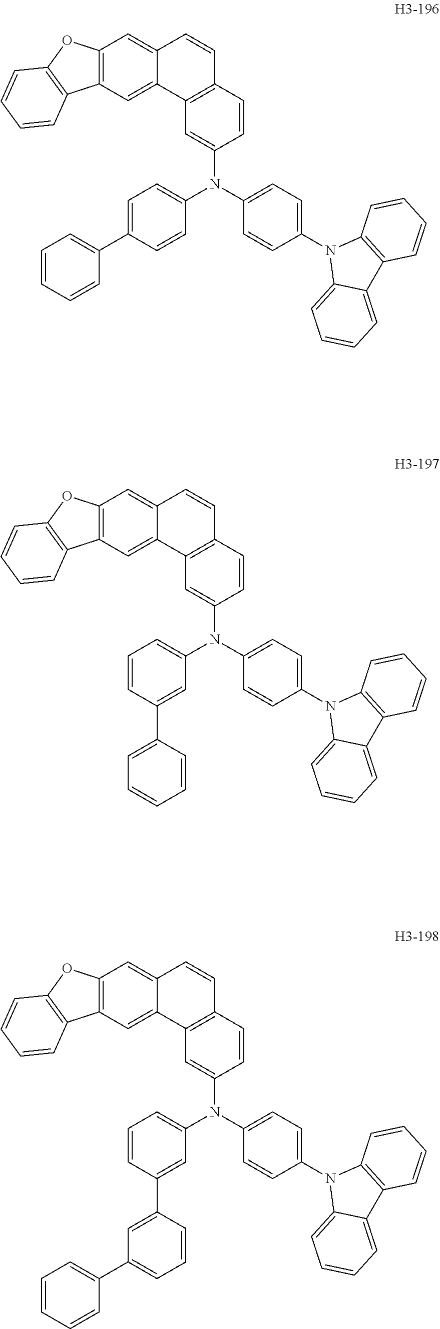 [SMILES: c1ccc(-c2ccc(N(c3ccc(-n4c5ccccc5c5ccccc54)cc3)c3ccc4ccc5cc6oc7ccccc7c6cc5c4c3)cc2)cc1 ; c1ccc(-c2cccc(N(c3ccc(-n4c5ccccc5c5ccccc54)cc3)c3ccc4ccc5cc6oc7ccccc7c6cc5c4c3)c2)cc1 ; c1ccc(-c2cccc(-c3cccc(N(c4ccc(-n5c6ccccc6c6ccccc65)cc4)c4ccc5ccc6cc7oc8ccccc8c7cc6c5c4)c3)c2)cc1]

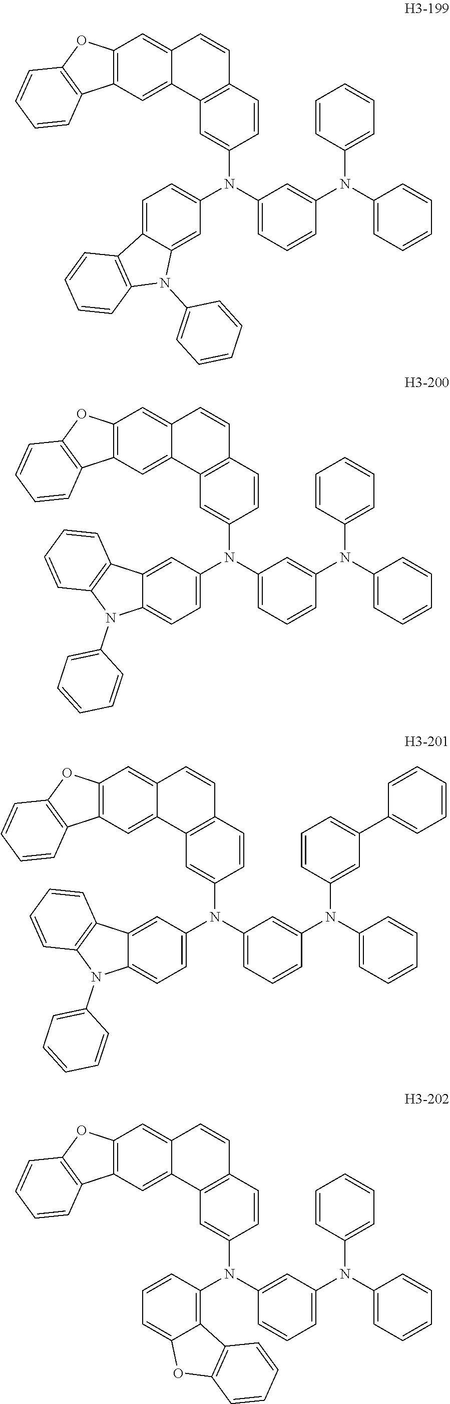

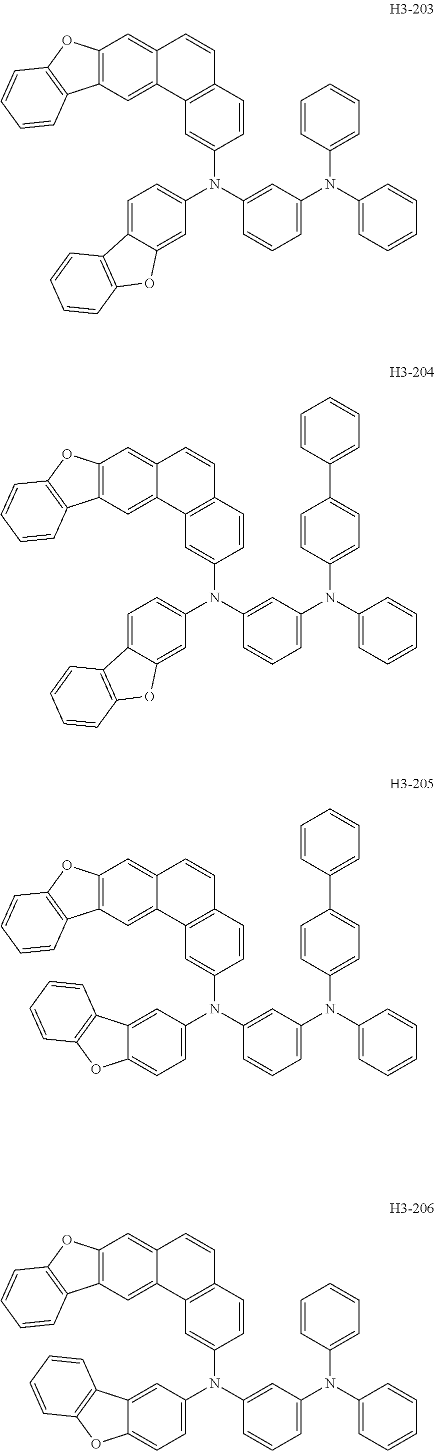 [SMILES: c1ccc(N(c2ccccc2)c2cccc(N(c3ccc4c(c3)oc3ccccc34)c3ccc4ccc5cc6oc7ccccc7c6cc5c4c3)c2)cc1 ; c1ccc(-c2ccc(N(c3ccccc3)c3cccc(N(c4ccc5c(c4)oc4ccccc45)c4ccc5ccc6cc7oc8ccccc8c7cc6c5c4)c3)cc2)cc1 ; c1ccc(-c2ccc(N(c3ccccc3)c3cccc(N(c4ccc5ccc6cc7oc8ccccc8c7cc6c5c4)c4ccc5oc6ccccc6c5c4)c3)cc2)cc1 ; c1ccc(N(c2ccccc2)c2cccc(N(c3ccc4ccc5cc6oc7ccccc7c6cc5c4c3)c3ccc4oc5ccccc5c4c3)c2)cc1]

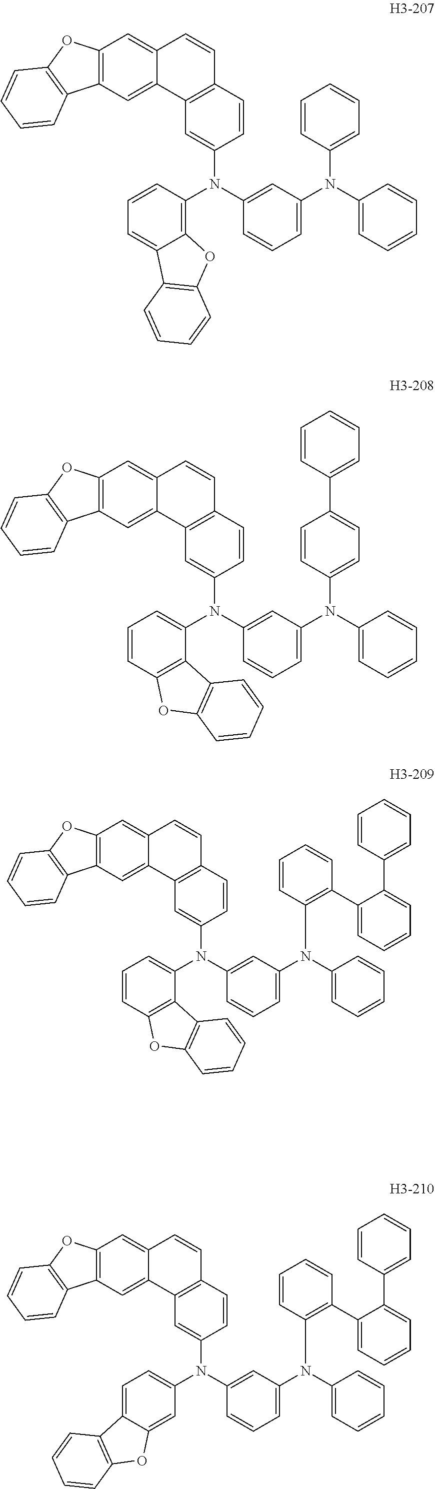 [SMILES: c1ccc(N(c2ccccc2)c2cccc(N(c3ccc4ccc5cc6oc7ccccc7c6cc5c4c3)c3cccc4c3oc3ccccc34)c2)cc1 ; c1ccc(-c2ccc(N(c3ccccc3)c3cccc(N(c4ccc5ccc6cc7oc8ccccc8c7cc6c5c4)c4cccc5oc6ccccc6c45)c3)cc2)cc1 ; c1ccc(-c2ccccc2-c2ccccc2N(c2ccccc2)c2cccc(N(c3ccc4ccc5cc6oc7ccccc7c6cc5c4c3)c3cccc4oc5ccccc5c34)c2)cc1 ; c1ccc(-c2ccccc2-c2ccccc2N(c2ccccc2)c2cccc(N(c3ccc4c(c3)oc3ccccc34)c3ccc4ccc5cc6oc7ccccc7c6cc5c4c3)c2)cc1]

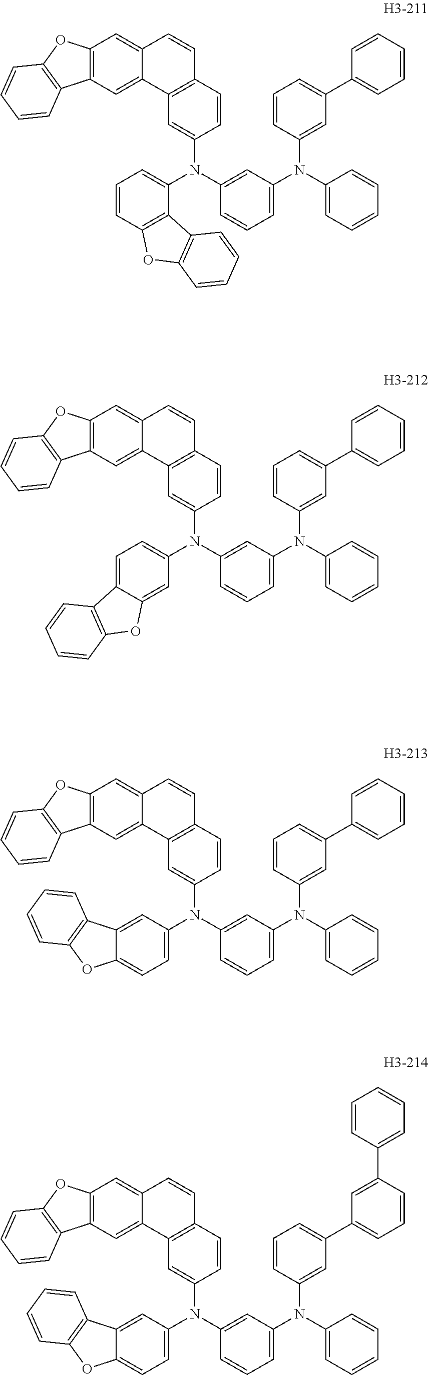 [SMILES: c1ccc(-c2cccc(N(c3ccccc3)c3cccc(N(c4ccc5ccc6cc7oc8ccccc8c7cc6c5c4)c4cccc5oc6ccccc6c45)c3)c2)cc1 ; c1ccc(-c2cccc(N(c3ccccc3)c3cccc(N(c4ccc5c(c4)oc4ccccc45)c4ccc5ccc6cc7oc8ccccc8c7cc6c5c4)c3)c2)cc1 ; c1ccc(-c2cccc(N(c3ccccc3)c3cccc(N(c4ccc5ccc6cc7oc8ccccc8c7cc6c5c4)c4ccc5oc6ccccc6c5c4)c3)c2)cc1 ; c1ccc(-c2cccc(-c3cccc(N(c4ccccc4)c4cccc(N(c5ccc6ccc7cc8oc9ccccc9c8cc7c6c5)c5ccc6oc7ccccc7c6c5)c4)c3)c2)cc1]

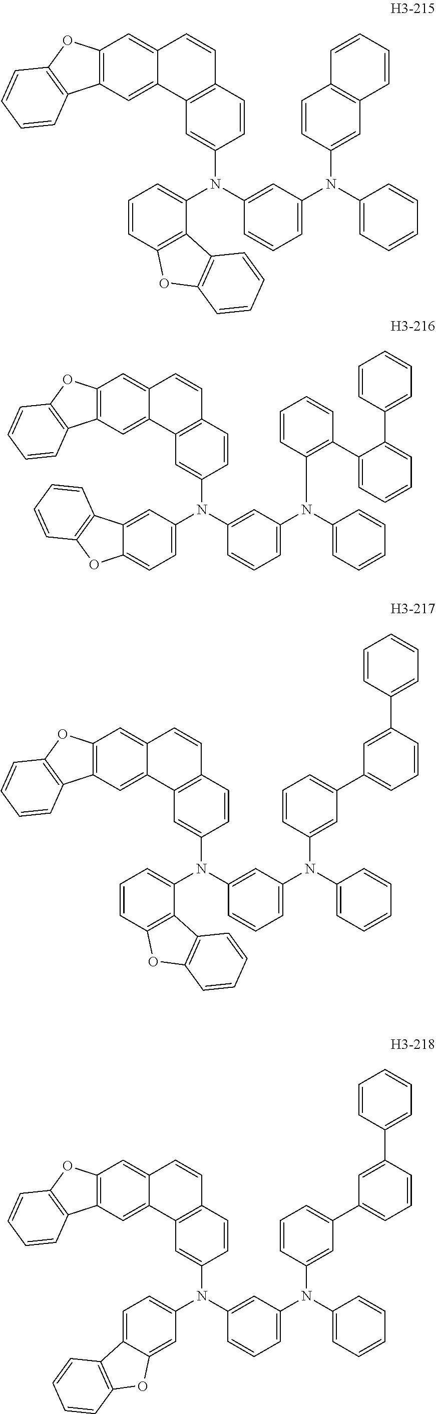 [SMILES: c1ccc(N(c2cccc(N(c3ccc4ccc5cc6oc7ccccc7c6cc5c4c3)c3cccc4oc5ccccc5c34)c2)c2ccc3ccccc3c2)cc1 ; c1ccc(-c2ccccc2-c2ccccc2N(c2ccccc2)c2cccc(N(c3ccc4ccc5cc6oc7ccccc7c6cc5c4c3)c3ccc4oc5ccccc5c4c3)c2)cc1 ; c1ccc(-c2cccc(-c3cccc(N(c4ccccc4)c4cccc(N(c5ccc6ccc7cc8oc9ccccc9c8cc7c6c5)c5cccc6oc7ccccc7c56)c4)c3)c2)cc1 ; c1ccc(-c2cccc(-c3cccc(N(c4ccccc4)c4cccc(N(c5ccc6c(c5)oc5ccccc56)c5ccc6ccc7cc8oc9ccccc9c8cc7c6c5)c4)c3)c2)cc1]

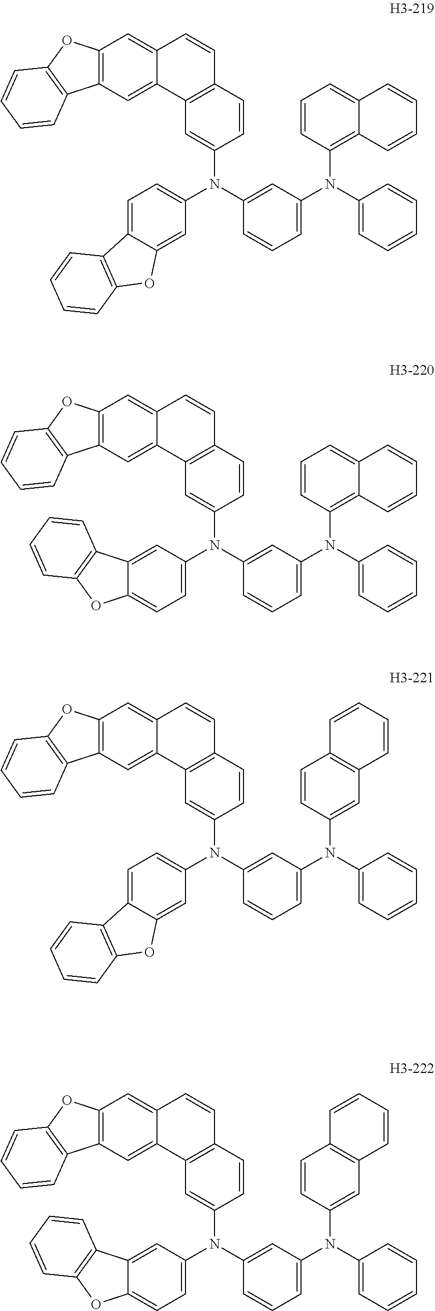 [SMILES: c1ccc(N(c2cccc(N(c3ccc4c(c3)oc3ccccc34)c3ccc4ccc5cc6oc7ccccc7c6cc5c4c3)c2)c2cccc3ccccc23)cc1 ; c1ccc(N(c2cccc(N(c3ccc4ccc5cc6oc7ccccc7c6cc5c4c3)c3ccc4oc5ccccc5c4c3)c2)c2cccc3ccccc23)cc1 ; c1ccc(N(c2cccc(N(c3ccc4c(c3)oc3ccccc34)c3ccc4ccc5cc6oc7ccccc7c6cc5c4c3)c2)c2ccc3ccccc3c2)cc1 ; c1ccc(N(c2cccc(N(c3ccc4ccc5cc6oc7ccccc7c6cc5c4c3)c3ccc4oc5ccccc5c4c3)c2)c2ccc3ccccc3c2)cc1]

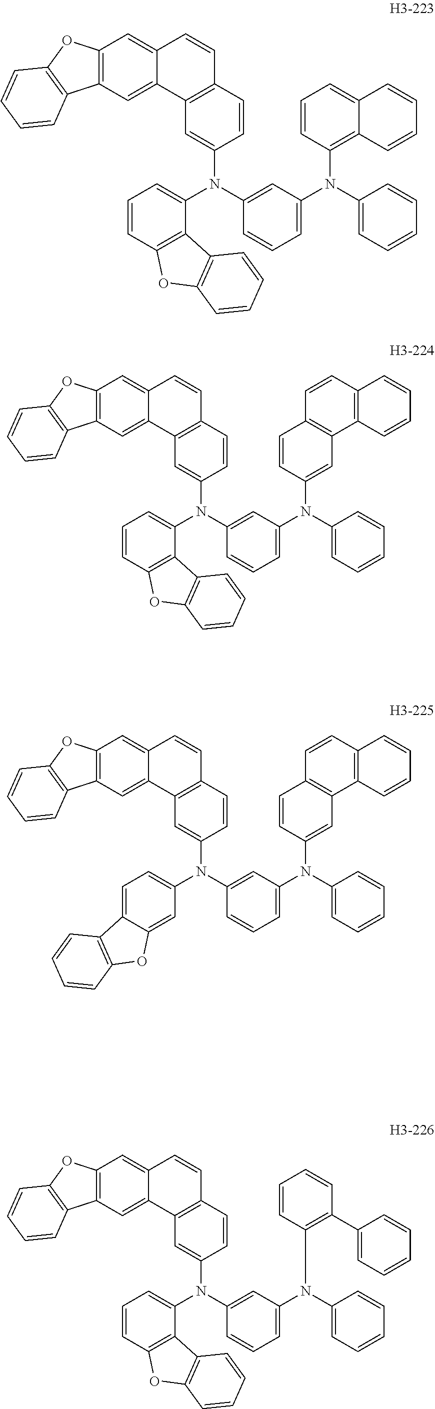 [SMILES: c1ccc(N(c2cccc(N(c3ccc4ccc5cc6oc7ccccc7c6cc5c4c3)c3cccc4oc5ccccc5c34)c2)c2cccc3ccccc23)cc1 ; c1ccc(N(c2cccc(N(c3ccc4ccc5cc6oc7ccccc7c6cc5c4c3)c3cccc4oc5ccccc5c34)c2)c2ccc3ccc4ccccc4c3c2)cc1 ; c1ccc(N(c2cccc(N(c3ccc4c(c3)oc3ccccc34)c3ccc4ccc5cc6oc7ccccc7c6cc5c4c3)c2)c2ccc3ccc4ccccc4c3c2)cc1 ; c1ccc(-c2ccccc2N(c2ccccc2)c2cccc(N(c3ccc4ccc5cc6oc7ccccc7c6cc5c4c3)c3cccc4oc5ccccc5c34)c2)cc1]

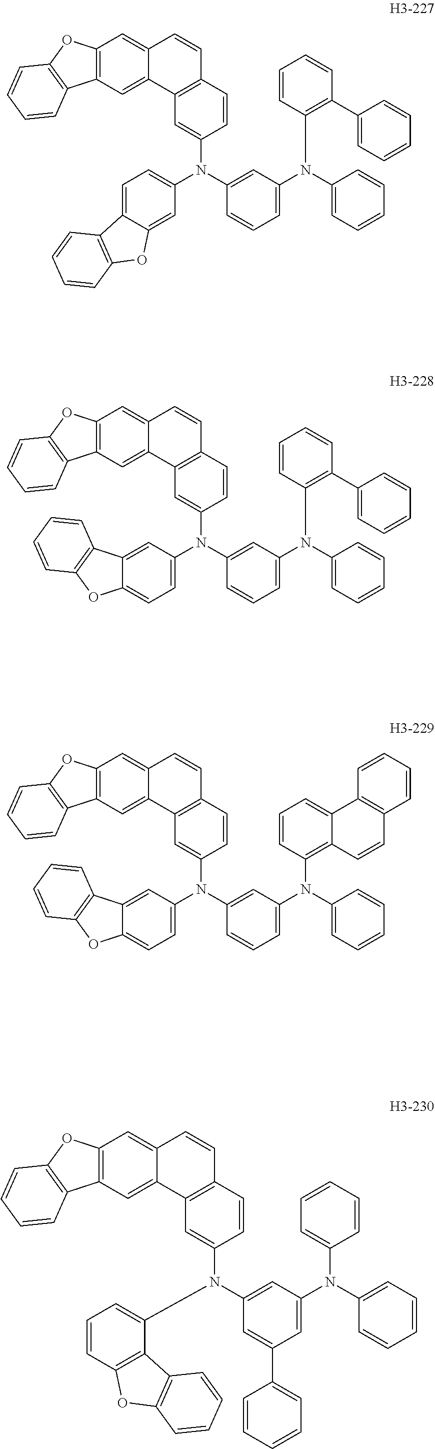 [SMILES: c1ccc(-c2ccccc2N(c2ccccc2)c2cccc(N(c3ccc4c(c3)oc3ccccc34)c3ccc4ccc5cc6oc7ccccc7c6cc5c4c3)c2)cc1 ; c1ccc(-c2ccccc2N(c2ccccc2)c2cccc(N(c3ccc4ccc5cc6oc7ccccc7c6cc5c4c3)c3ccc4oc5ccccc5c4c3)c2)cc1 ; c1ccc(N(c2cccc(N(c3ccc4ccc5cc6oc7ccccc7c6cc5c4c3)c3ccc4oc5ccccc5c4c3)c2)c2cccc3c2ccc2ccccc23)cc1 ; c1ccc(-c2cc(N(c3ccccc3)c3ccccc3)cc(N(c3ccc4ccc5cc6oc7ccccc7c6cc5c4c3)c3cccc4oc5ccccc5c34)c2)cc1]

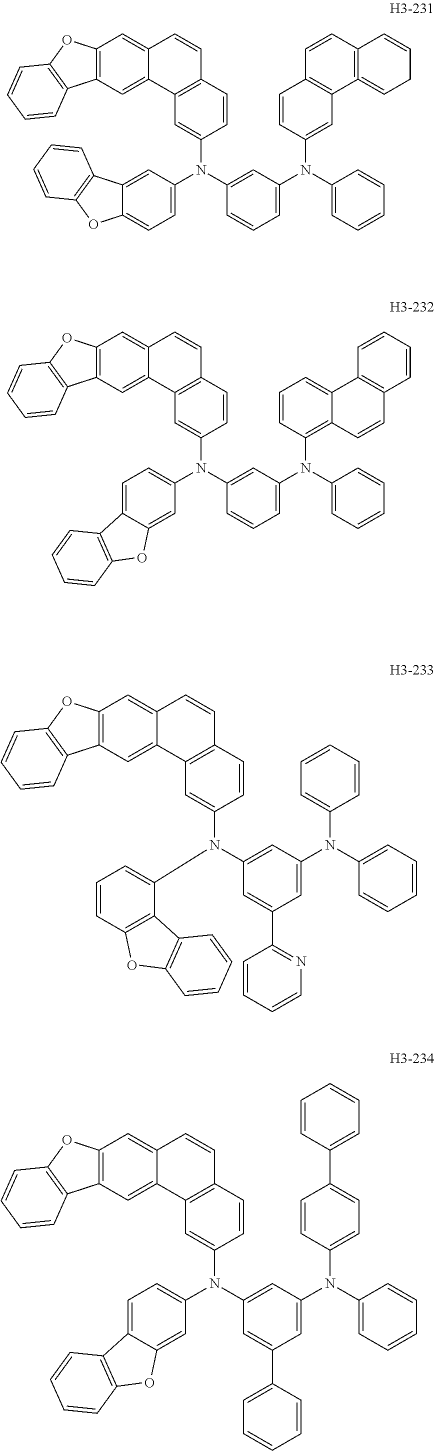

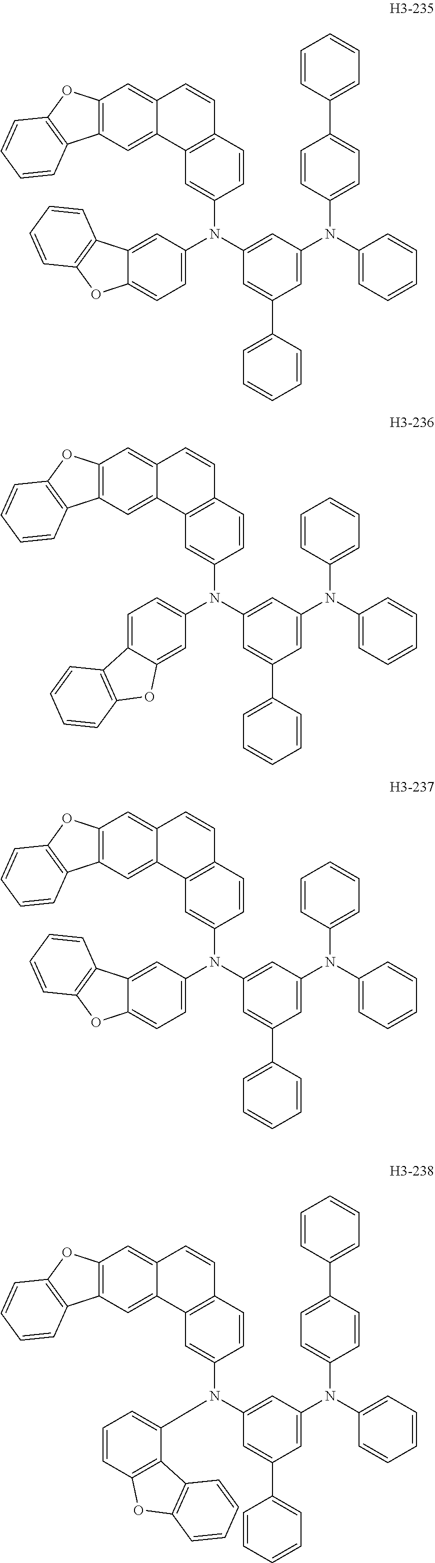 [SMILES: c1ccc(-c2ccc(N(c3ccccc3)c3cc(-c4ccccc4)cc(N(c4ccc5ccc6cc7oc8ccccc8c7cc6c5c4)c4ccc5oc6ccccc6c5c4)c3)cc2)cc1 ; c1ccc(-c2cc(N(c3ccccc3)c3ccccc3)cc(N(c3ccc4c(c3)oc3ccccc34)c3ccc4ccc5cc6oc7ccccc7c6cc5c4c3)c2)cc1 ; c1ccc(-c2cc(N(c3ccccc3)c3ccccc3)cc(N(c3ccc4ccc5cc6oc7ccccc7c6cc5c4c3)c3ccc4oc5ccccc5c4c3)c2)cc1 ; c1ccc(-c2ccc(N(c3ccccc3)c3cc(-c4ccccc4)cc(N(c4ccc5ccc6cc7oc8ccccc8c7cc6c5c4)c4cccc5oc6ccccc6c45)c3)cc2)cc1]

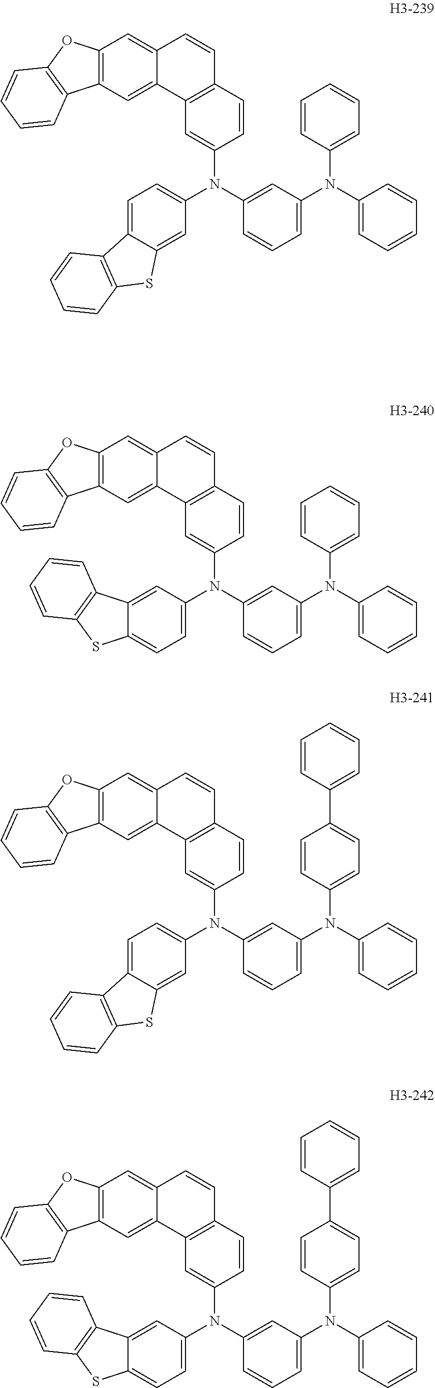 [SMILES: c1ccc(N(c2ccccc2)c2cccc(N(c3ccc4c(c3)sc3ccccc34)c3ccc4ccc5cc6oc7ccccc7c6cc5c4c3)c2)cc1 ; c1ccc(N(c2ccccc2)c2cccc(N(c3ccc4ccc5cc6oc7ccccc7c6cc5c4c3)c3ccc4sc5ccccc5c4c3)c2)cc1 ; c1ccc(-c2ccc(N(c3ccccc3)c3cccc(N(c4ccc5c(c4)sc4ccccc45)c4ccc5ccc6cc7oc8ccccc8c7cc6c5c4)c3)cc2)cc1 ; c1ccc(-c2ccc(N(c3ccccc3)c3cccc(N(c4ccc5ccc6cc7oc8ccccc8c7cc6c5c4)c4ccc5sc6ccccc6c5c4)c3)cc2)cc1]

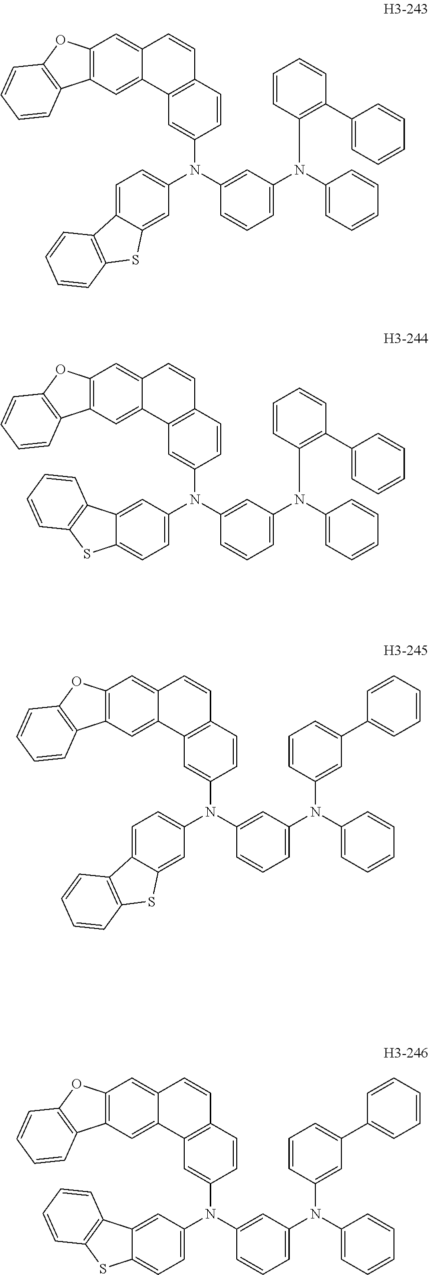 [SMILES: c1ccc(-c2ccccc2N(c2ccccc2)c2cccc(N(c3ccc4c(c3)sc3ccccc34)c3ccc4ccc5cc6oc7ccccc7c6cc5c4c3)c2)cc1 ; c1ccc(-c2ccccc2N(c2ccccc2)c2cccc(N(c3ccc4ccc5cc6oc7ccccc7c6cc5c4c3)c3ccc4sc5ccccc5c4c3)c2)cc1 ; c1ccc(-c2cccc(N(c3ccccc3)c3cccc(N(c4ccc5c(c4)sc4ccccc45)c4ccc5ccc6cc7oc8ccccc8c7cc6c5c4)c3)c2)cc1 ; c1ccc(-c2cccc(N(c3ccccc3)c3cccc(N(c4ccc5ccc6cc7oc8ccccc8c7cc6c5c4)c4ccc5sc6ccccc6c5c4)c3)c2)cc1]

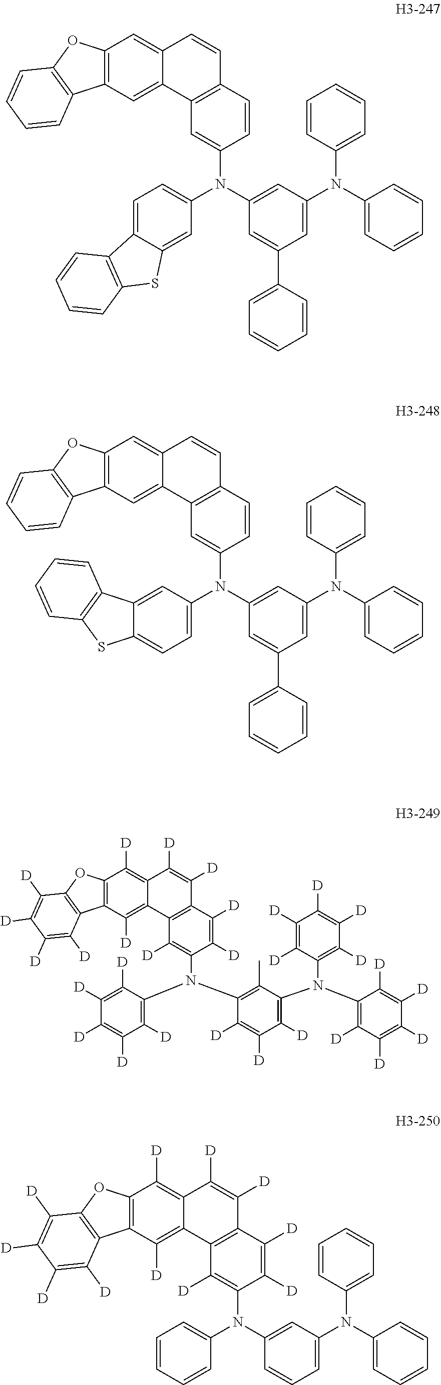

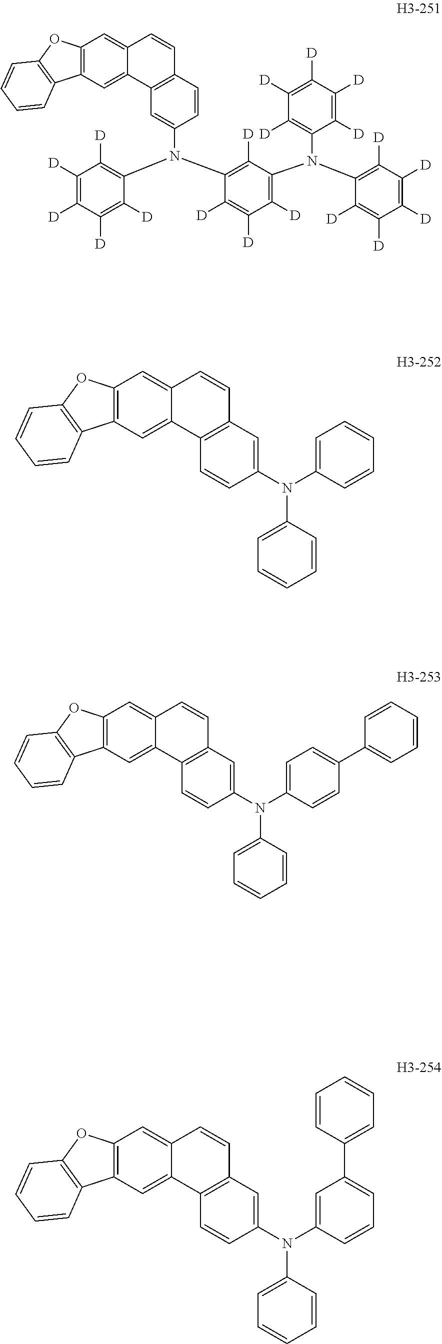 [SMILES: [2H]c1c([2H])c([2H])c(N(c2ccc3ccc4cc5oc6ccccc6c5cc4c3c2)c2c([2H])c([2H])c([2H])c(N(c3c([2H])c([2H])c([2H])c([2H])c3[2H])c3c([2H])c([2H])c([2H])c([2H])c3[2H])c2[2H])c([2H])c1[2H] ; c1ccc(N(c2ccccc2)c2ccc3c(ccc4cc5oc6ccccc6c5cc43)c2)cc1 ; c1ccc(-c2ccc(N(c3ccccc3)c3ccc4c(ccc5cc6oc7ccccc7c6cc54)c3)cc2)cc1 ; c1ccc(-c2cccc(N(c3ccccc3)c3ccc4c(ccc5cc6oc7ccccc7c6cc54)c3)c2)cc1]

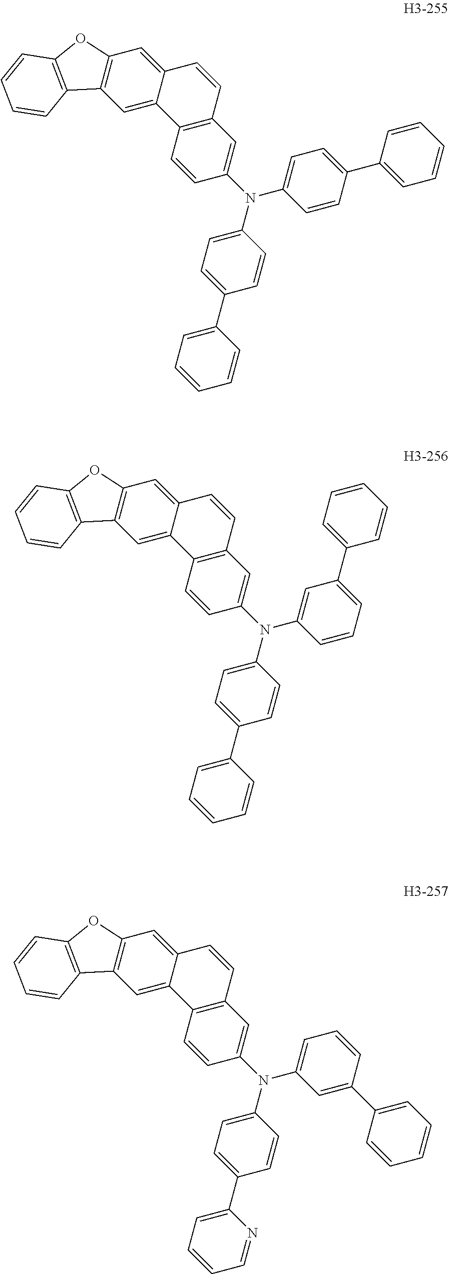 [SMILES: c1ccc(-c2ccc(N(c3ccc(-c4ccccc4)cc3)c3ccc4c(ccc5cc6oc7ccccc7c6cc54)c3)cc2)cc1 ; c1ccc(-c2ccc(N(c3cccc(-c4ccccc4)c3)c3ccc4c(ccc5cc6oc7ccccc7c6cc54)c3)cc2)cc1 ; c1ccc(-c2cccc(N(c3ccc(-c4ccccn4)cc3)c3ccc4c(ccc5cc6oc7ccccc7c6cc54)c3)c2)cc1]

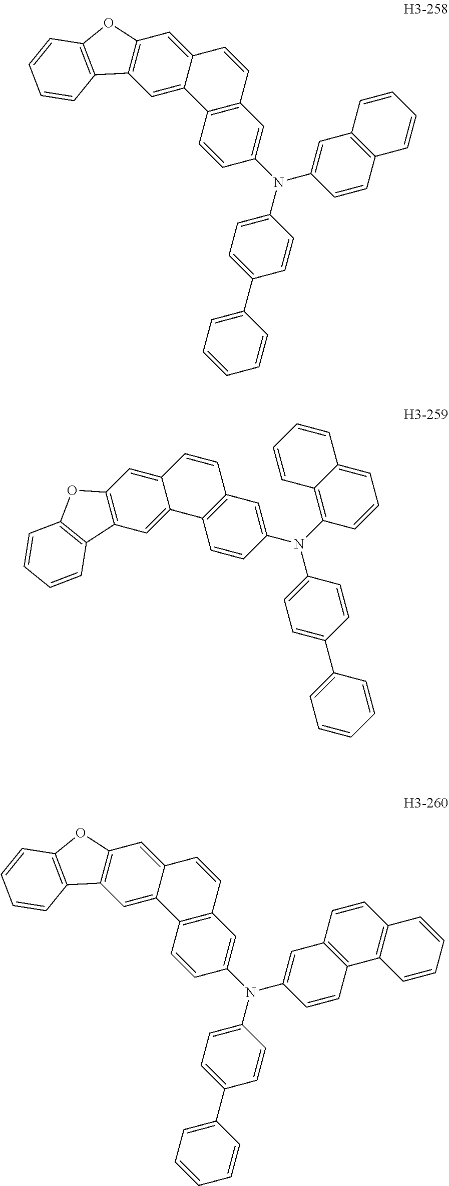 [SMILES: c1ccc(-c2ccc(N(c3ccc4ccccc4c3)c3ccc4c(ccc5cc6oc7ccccc7c6cc54)c3)cc2)cc1 ; c1ccc(-c2ccc(N(c3ccc4c(ccc5cc6oc7ccccc7c6cc54)c3)c3cccc4ccccc34)cc2)cc1 ; c1ccc(-c2ccc(N(c3ccc4c(ccc5ccccc54)c3)c3ccc4c(ccc5cc6oc7ccccc7c6cc54)c3)cc2)cc1]

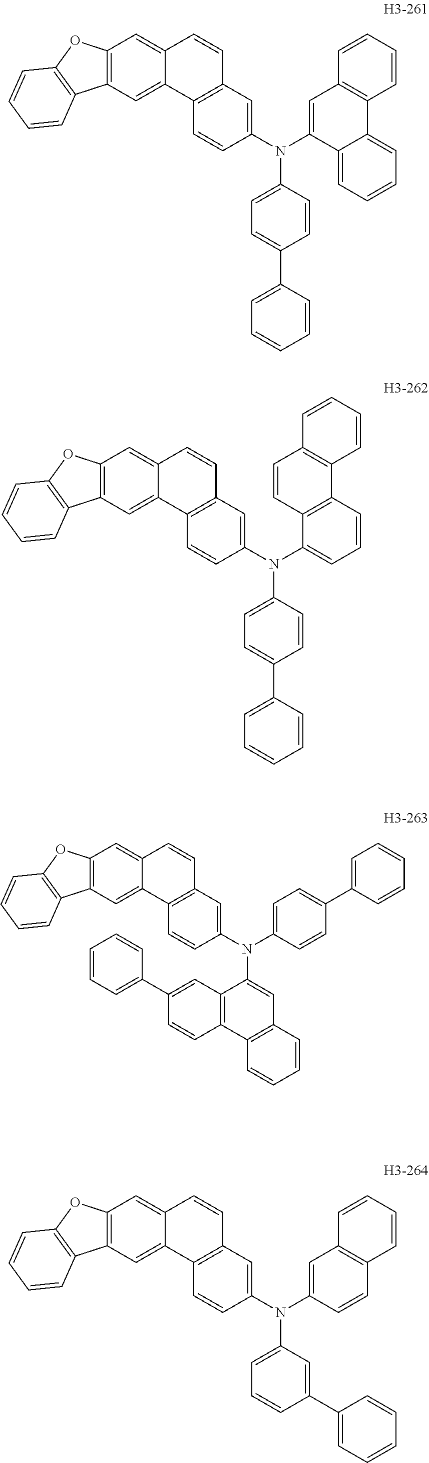 [SMILES: c1ccc(-c2ccc(N(c3ccc4c(ccc5cc6oc7ccccc7c6cc54)c3)c3cc4ccccc4c4ccccc34)cc2)cc1 ; c1ccc(-c2ccc(N(c3ccc4c(ccc5cc6oc7ccccc7c6cc54)c3)c3cccc4c3ccc3ccccc34)cc2)cc1 ; c1ccc(-c2ccc(N(c3ccc4c(ccc5cc6oc7ccccc7c6cc54)c3)c3cc4ccccc4c4ccc(-c5ccccc5)cc34)cc2)cc1 ; c1ccc(-c2cccc(N(c3ccc4ccccc4c3)c3ccc4c(ccc5cc6oc7ccccc7c6cc54)c3)c2)cc1]

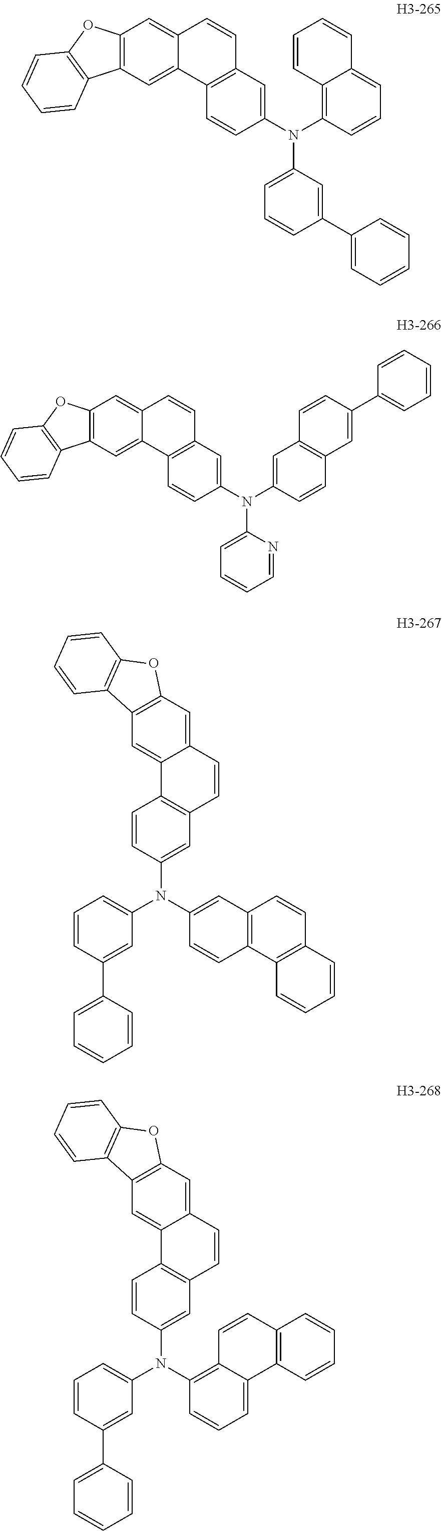

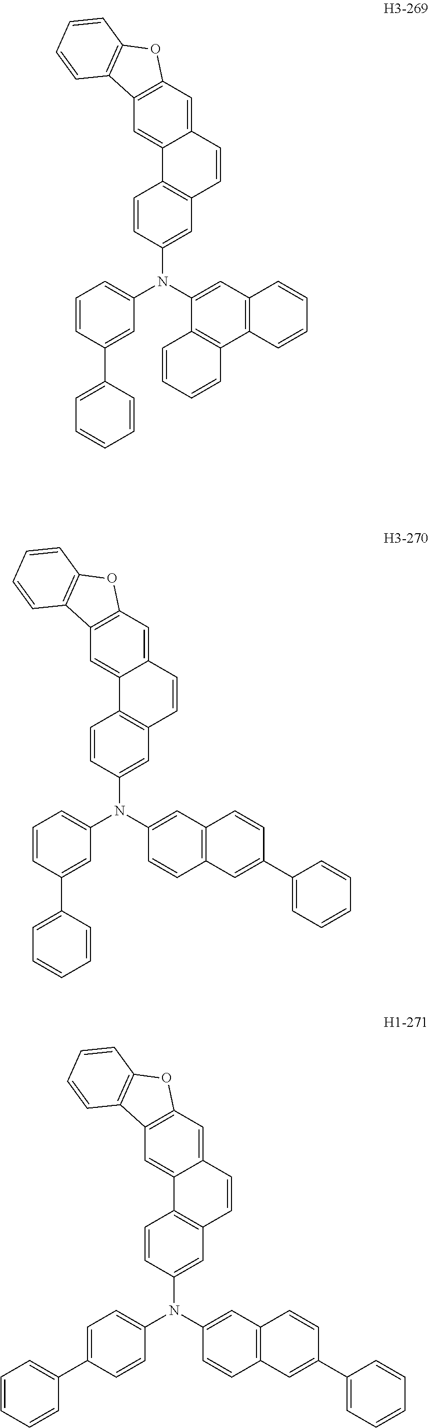 [SMILES: c1ccc(-c2cccc(N(c3ccc4c(ccc5cc6oc7ccccc7c6cc54)c3)c3cc4ccccc4c4ccccc34)c2)cc1 ; c1ccc(-c2cccc(N(c3ccc4cc(-c5ccccc5)ccc4c3)c3ccc4c(ccc5cc6oc7ccccc7c6cc54)c3)c2)cc1 ; c1ccc(-c2ccc(N(c3ccc4cc(-c5ccccc5)ccc4c3)c3ccc4c(ccc5cc6oc7ccccc7c6cc54)c3)cc2)cc1]

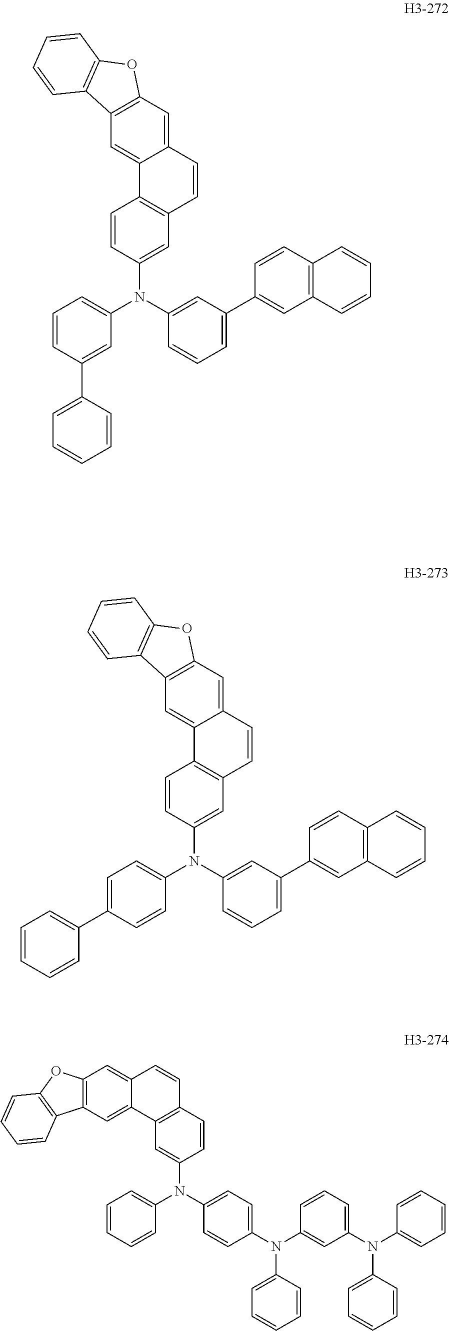 [SMILES: c1ccc(-c2cccc(N(c3cccc(-c4ccc5ccccc5c4)c3)c3ccc4c(ccc5cc6oc7ccccc7c6cc54)c3)c2)cc1 ; c1ccc(-c2ccc(N(c3cccc(-c4ccc5ccccc5c4)c3)c3ccc4c(ccc5cc6oc7ccccc7c6cc54)c3)cc2)cc1 ; c1ccc(N(c2ccccc2)c2cccc(N(c3ccccc3)c3ccc(N(c4ccccc4)c4ccc5ccc6cc7oc8ccccc8c7cc6c5c4)cc3)c2)cc1]

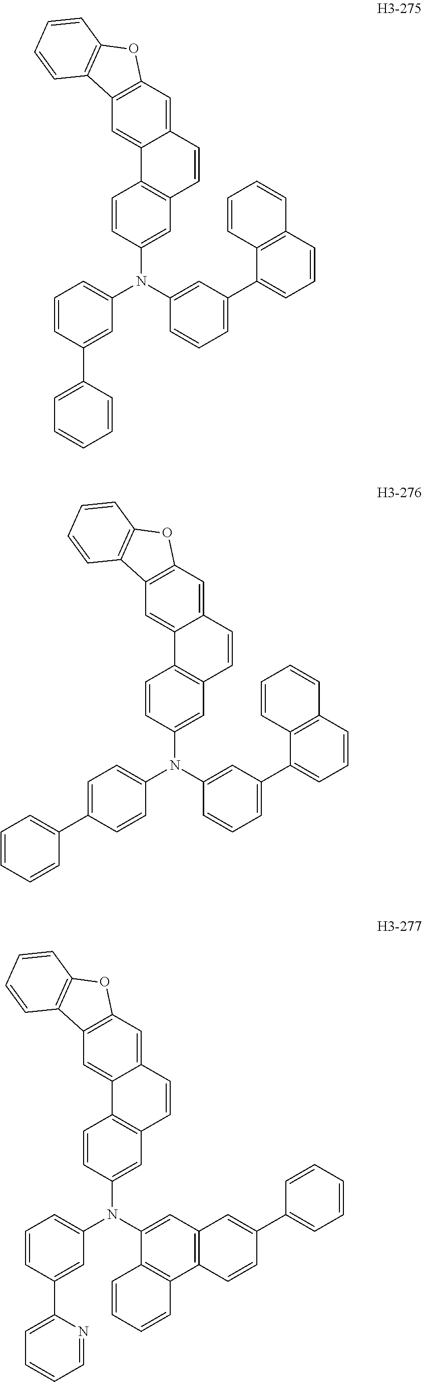 [SMILES: c1ccc(-c2cccc(N(c3cccc(-c4cccc5ccccc45)c3)c3ccc4c(ccc5cc6oc7ccccc7c6cc54)c3)c2)cc1 ; c1ccc(-c2ccc(N(c3cccc(-c4cccc5ccccc45)c3)c3ccc4c(ccc5cc6oc7ccccc7c6cc54)c3)cc2)cc1 ; c1ccc(-c2ccc3c(c2)cc(N(c2cccc(-c4ccccn4)c2)c2ccc4c(ccc5cc6oc7ccccc7c6cc54)c2)c2ccccc23)cc1]

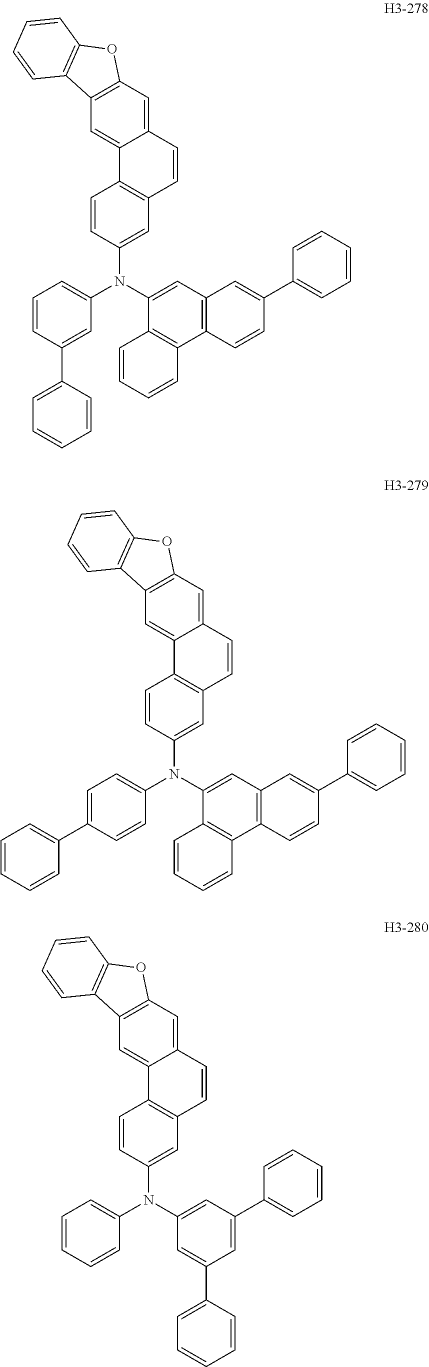 [SMILES: c1ccc(-c2cccc(N(c3ccc4c(ccc5cc6oc7ccccc7c6cc54)c3)c3cc4cc(-c5ccccc5)ccc4c4ccccc34)c2)cc1 ; c1ccc(-c2ccc(N(c3ccc4c(ccc5cc6oc7ccccc7c6cc54)c3)c3cc4cc(-c5ccccc5)ccc4c4ccccc34)cc2)cc1 ; c1ccc(-c2cc(-c3ccccc3)cc(N(c3ccccc3)c3ccc4c(ccc5cc6oc7ccccc7c6cc54)c3)c2)cc1]

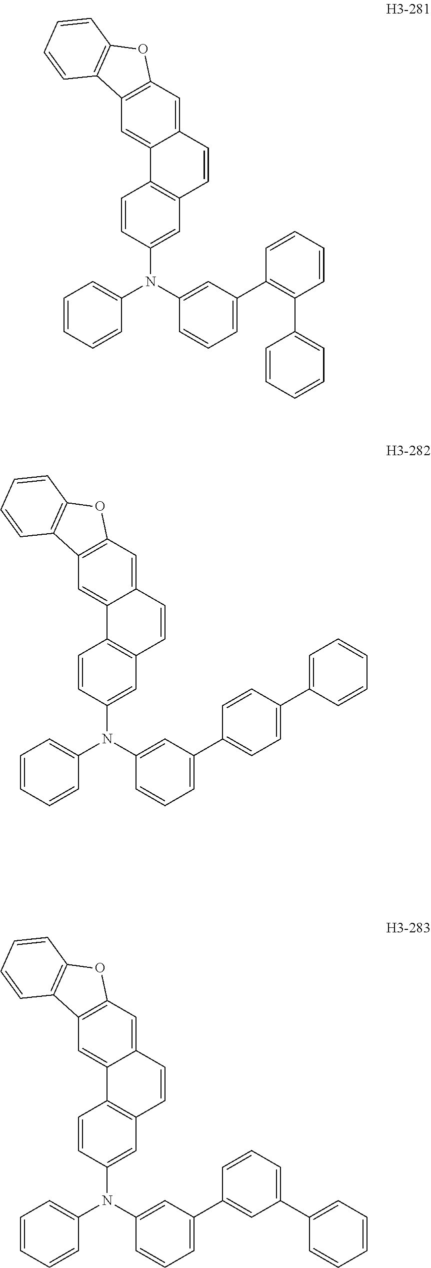 [SMILES: c1ccc(-c2ccccc2-c2cccc(N(c3ccccc3)c3ccc4c(ccc5cc6oc7ccccc7c6cc54)c3)c2)cc1 ; c1ccc(-c2ccc(-c3cccc(N(c4ccccc4)c4ccc5c(ccc6cc7oc8ccccc8c7cc65)c4)c3)cc2)cc1 ; c1ccc(-c2cccc(-c3cccc(N(c4ccccc4)c4ccc5c(ccc6cc7oc8ccccc8c7cc65)c4)c3)c2)cc1]

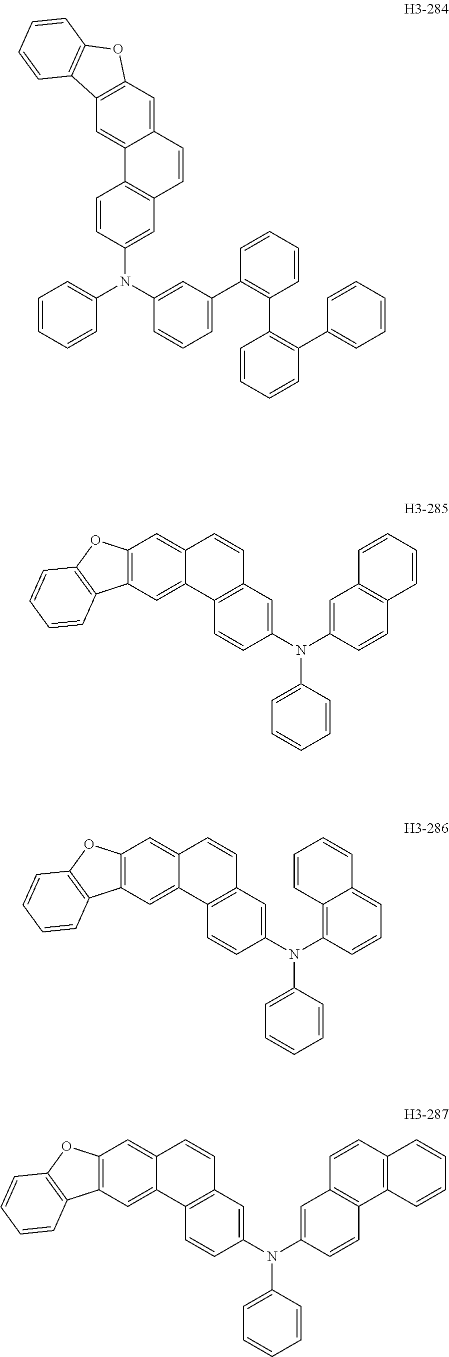 [SMILES: c1ccc(-c2ccccc2-c2ccccc2-c2cccc(N(c3ccccc3)c3ccc4c(ccc5cc6oc7ccccc7c6cc54)c3)c2)cc1 ; c1ccc(N(c2ccc3ccccc3c2)c2ccc3c(ccc4cc5oc6ccccc6c5cc43)c2)cc1 ; c1ccc(N(c2ccc3c(ccc4cc5oc6ccccc6c5cc43)c2)c2cccc3ccccc23)cc1 ; c1ccc(N(c2ccc3c(ccc4ccccc43)c2)c2ccc3c(ccc4cc5oc6ccccc6c5cc43)c2)cc1]

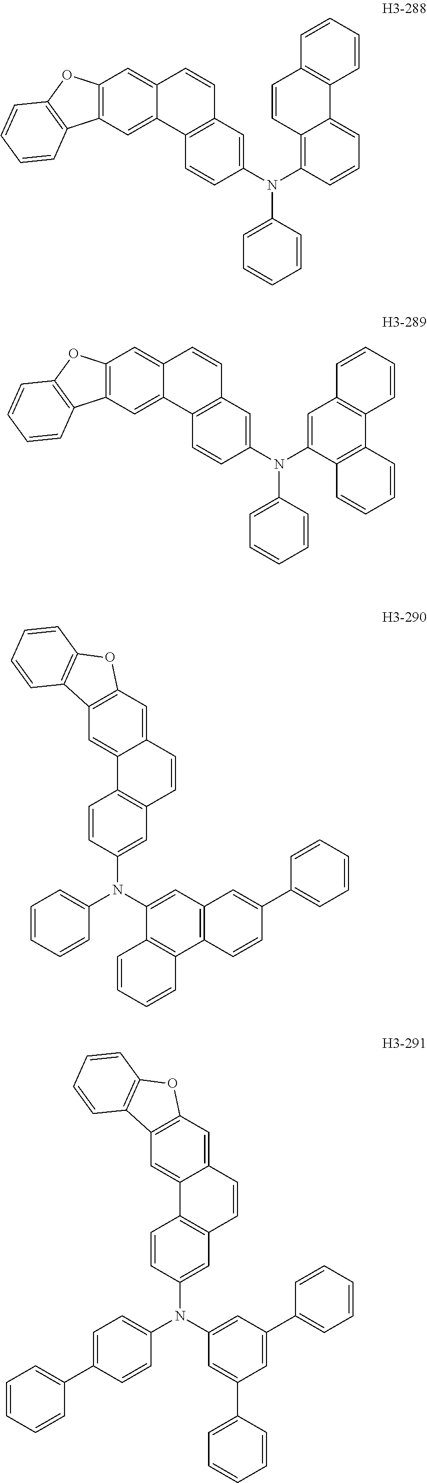 [SMILES: c1ccc(N(c2ccc3c(ccc4cc5oc6ccccc6c5cc43)c2)c2cccc3c2ccc2ccccc23)cc1 ; c1ccc(N(c2ccc3c(ccc4cc5oc6ccccc6c5cc43)c2)c2cc3ccccc3c3ccccc23)cc1 ; c1ccc(-c2ccc3c(c2)cc(N(c2ccccc2)c2ccc4c(ccc5cc6oc7ccccc7c6cc54)c2)c2ccccc23)cc1 ; c1ccc(-c2ccc(N(c3cc(-c4ccccc4)cc(-c4ccccc4)c3)c3ccc4c(ccc5cc6oc7ccccc7c6cc54)c3)cc2)cc1]

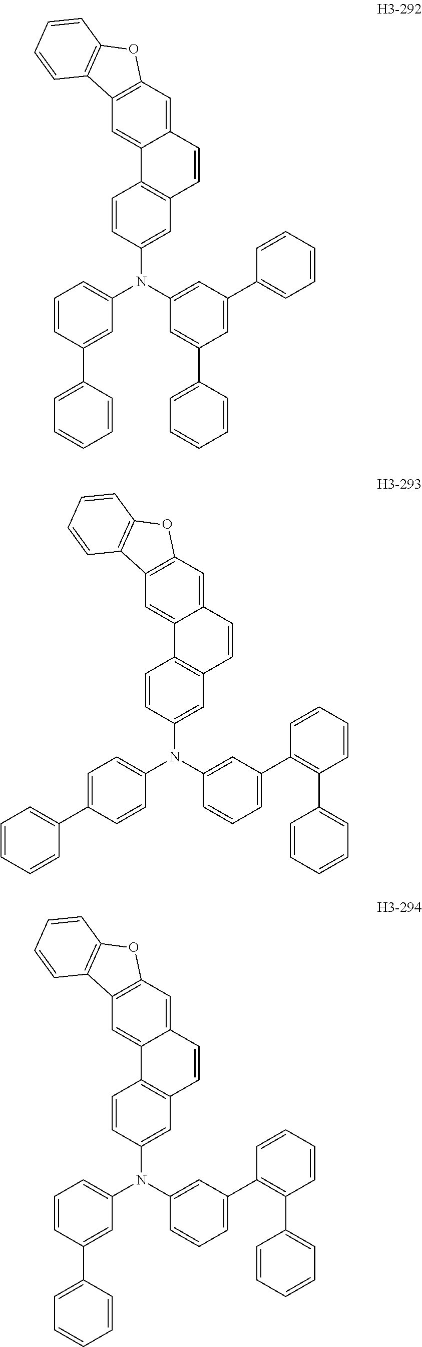 [SMILES: c1ccc(-c2cccc(N(c3cc(-c4ccccc4)cc(-c4ccccc4)c3)c3ccc4c(ccc5cc6oc7ccccc7c6cc54)c3)c2)cc1 ; c1ccc(-c2ccc(N(c3cccc(-c4ccccc4-c4ccccc4)c3)c3ccc4c(ccc5cc6oc7ccccc7c6cc54)c3)cc2)cc1 ; c1ccc(-c2cccc(N(c3cccc(-c4ccccc4-c4ccccc4)c3)c3ccc4c(ccc5cc6oc7ccccc7c6cc54)c3)c2)cc1]

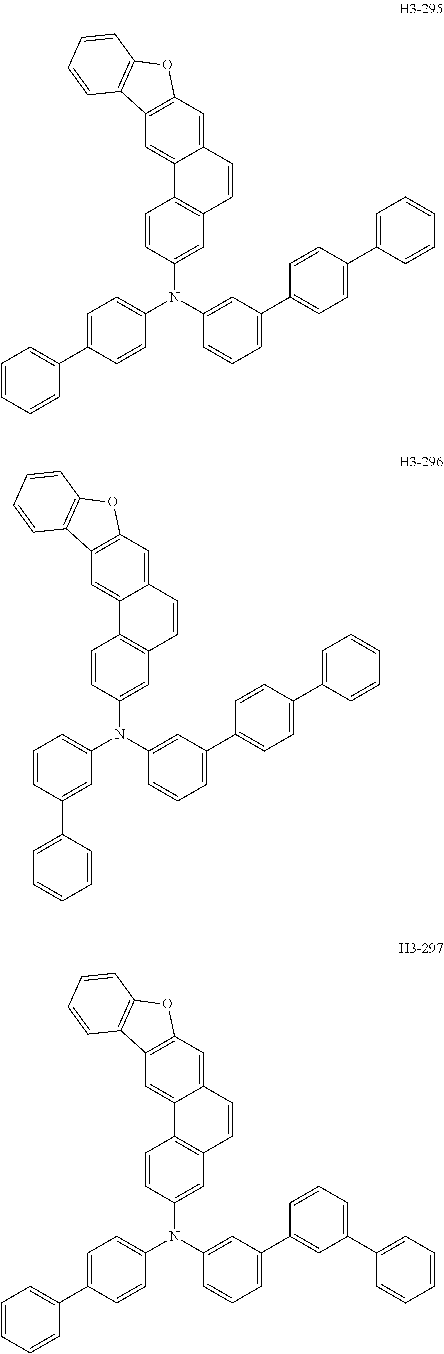 [SMILES: c1ccc(-c2ccc(-c3cccc(N(c4ccc(-c5ccccc5)cc4)c4ccc5c(ccc6cc7oc8ccccc8c7cc65)c4)c3)cc2)cc1 ; c1ccc(-c2ccc(-c3cccc(N(c4cccc(-c5ccccc5)c4)c4ccc5c(ccc6cc7oc8ccccc8c7cc65)c4)c3)cc2)cc1 ; c1ccc(-c2ccc(N(c3cccc(-c4cccc(-c5ccccc5)c4)c3)c3ccc4c(ccc5cc6oc7ccccc7c6cc54)c3)cc2)cc1]

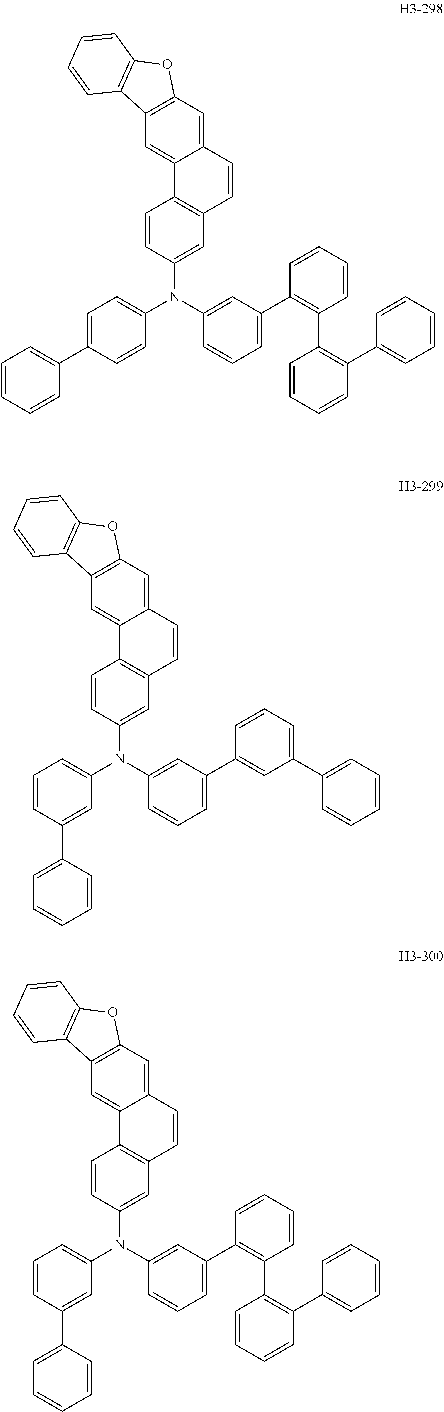 [SMILES: c1ccc(-c2ccc(N(c3cccc(-c4ccccc4-c4ccccc4-c4ccccc4)c3)c3ccc4c(ccc5cc6oc7ccccc7c6cc54)c3)cc2)cc1 ; c1ccc(-c2cccc(-c3cccc(N(c4cccc(-c5ccccc5)c4)c4ccc5c(ccc6cc7oc8ccccc8c7cc65)c4)c3)c2)cc1 ; c1ccc(-c2cccc(N(c3cccc(-c4ccccc4-c4ccccc4-c4ccccc4)c3)c3ccc4c(ccc5cc6oc7ccccc7c6cc54)c3)c2)cc1]

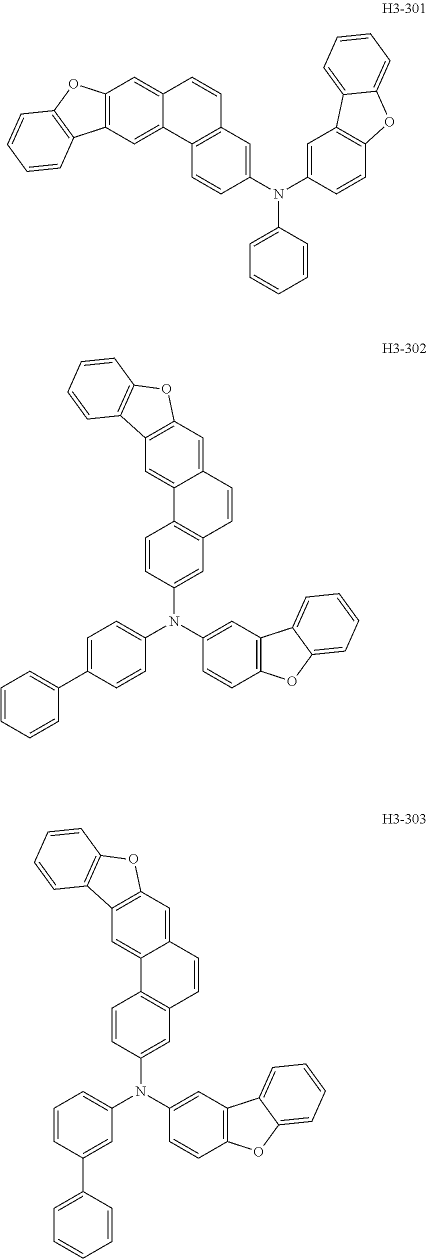 [SMILES: c1ccc(N(c2ccc3c(ccc4cc5oc6ccccc6c5cc43)c2)c2ccc3oc4ccccc4c3c2)cc1 ; c1ccc(-c2ccc(N(c3ccc4c(ccc5cc6oc7ccccc7c6cc54)c3)c3ccc4oc5ccccc5c4c3)cc2)cc1 ; c1ccc(-c2cccc(N(c3ccc4c(ccc5cc6oc7ccccc7c6cc54)c3)c3ccc4oc5ccccc5c4c3)c2)cc1]

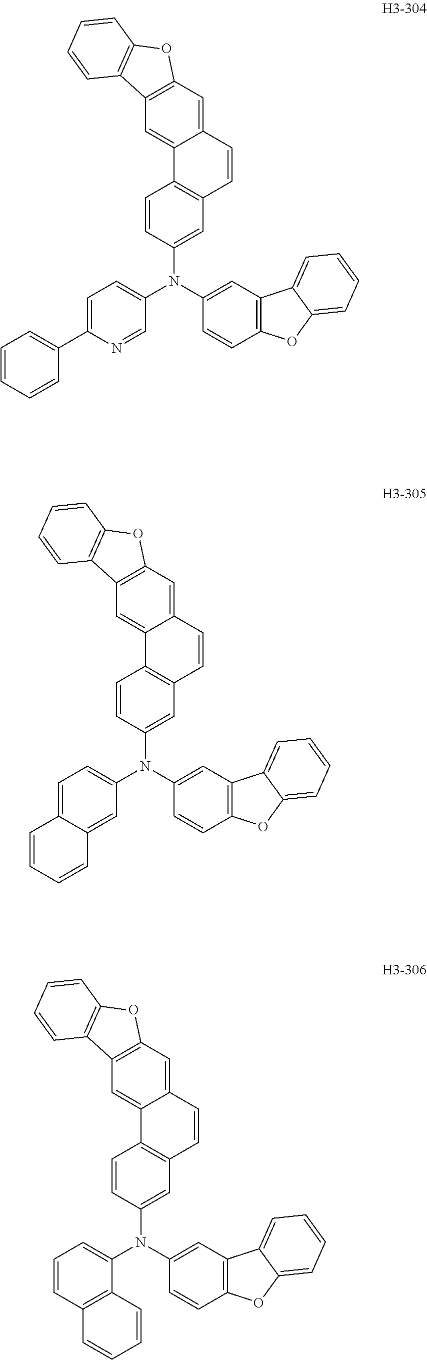 [SMILES: c1ccc(-c2ccc(N(c3ccc4c(ccc5cc6oc7ccccc7c6cc54)c3)c3ccc4oc5ccccc5c4c3)cn2)cc1 ; c1ccc2cc(N(c3ccc4c(ccc5cc6oc7ccccc7c6cc54)c3)c3ccc4oc5ccccc5c4c3)ccc2c1 ; c1ccc2c(N(c3ccc4c(ccc5cc6oc7ccccc7c6cc54)c3)c3ccc4oc5ccccc5c4c3)cccc2c1]

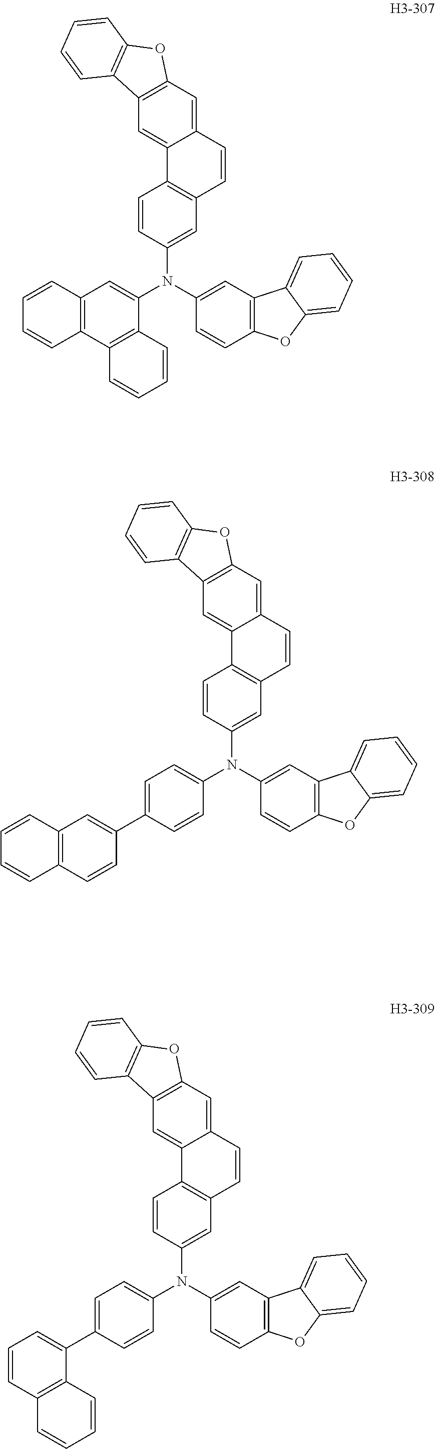 [SMILES: c1ccc2c(c1)cc(N(c1ccc3c(ccc4cc5oc6ccccc6c5cc43)c1)c1ccc3oc4ccccc4c3c1)c1ccccc12 ; c1ccc2cc(-c3ccc(N(c4ccc5c(ccc6cc7oc8ccccc8c7cc65)c4)c4ccc5oc6ccccc6c5c4)cc3)ccc2c1 ; c1ccc2c(-c3ccc(N(c4ccc5c(ccc6cc7oc8ccccc8c7cc65)c4)c4ccc5oc6ccccc6c5c4)cc3)cccc2c1]

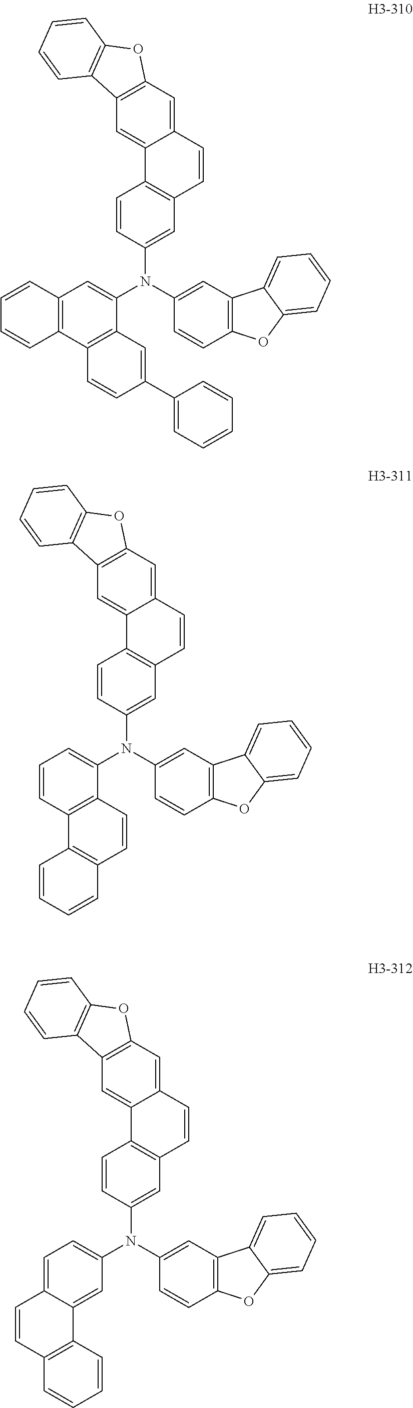 [SMILES: c1ccc(-c2ccc3c(c2)c(N(c2ccc4c(ccc5cc6oc7ccccc7c6cc54)c2)c2ccc4oc5ccccc5c4c2)cc2ccccc23)cc1 ; c1ccc2c(c1)ccc1c(N(c3ccc4c(ccc5cc6oc7ccccc7c6cc54)c3)c3ccc4oc5ccccc5c4c3)cccc12 ; c1ccc2c(c1)ccc1ccc(N(c3ccc4c(ccc5cc6oc7ccccc7c6cc54)c3)c3ccc4oc5ccccc5c4c3)cc12]

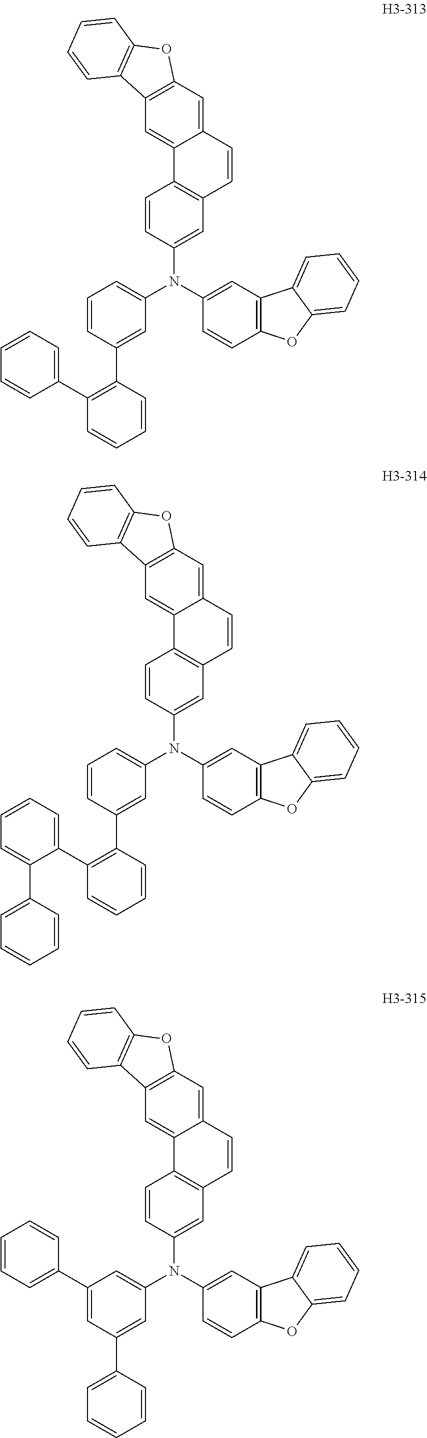

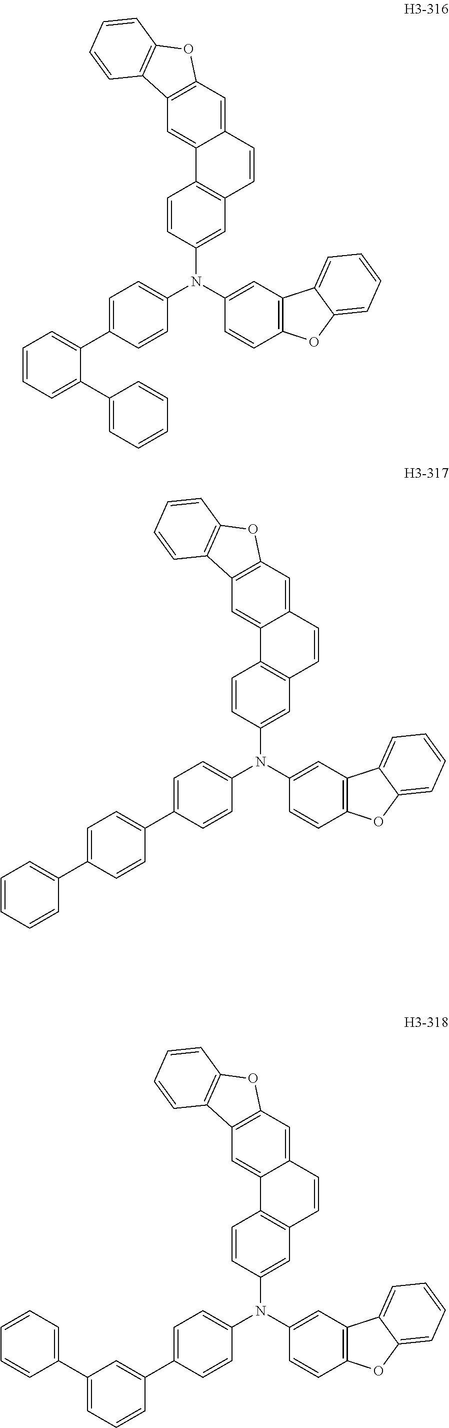 [SMILES: c1ccc(-c2ccccc2-c2ccc(N(c3ccc4c(ccc5cc6oc7ccccc7c6cc54)c3)c3ccc4oc5ccccc5c4c3)cc2)cc1 ; c1ccc(-c2ccc(-c3ccc(N(c4ccc5c(ccc6cc7oc8ccccc8c7cc65)c4)c4ccc5oc6ccccc6c5c4)cc3)cc2)cc1 ; c1ccc(-c2cccc(-c3ccc(N(c4ccc5c(ccc6cc7oc8ccccc8c7cc65)c4)c4ccc5oc6ccccc6c5c4)cc3)c2)cc1]

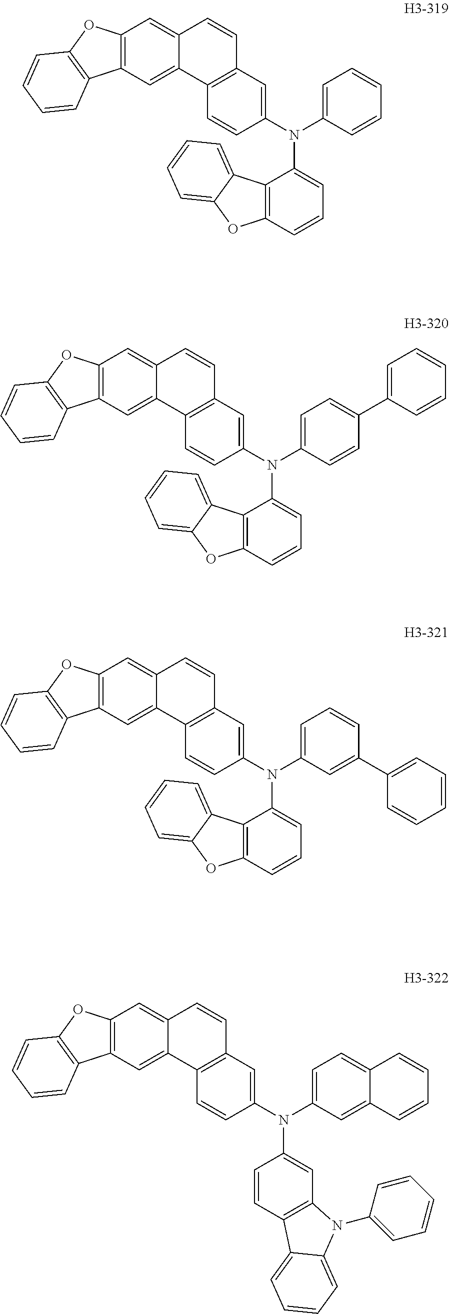 [SMILES: c1ccc(N(c2ccc3c(ccc4cc5oc6ccccc6c5cc43)c2)c2cccc3oc4ccccc4c23)cc1 ; c1ccc(-c2ccc(N(c3ccc4c(ccc5cc6oc7ccccc7c6cc54)c3)c3cccc4oc5ccccc5c34)cc2)cc1 ; c1ccc(-c2cccc(N(c3ccc4c(ccc5cc6oc7ccccc7c6cc54)c3)c3cccc4oc5ccccc5c34)c2)cc1 ; c1ccc(-n2c3ccccc3c3ccc(N(c4ccc5ccccc5c4)c4ccc5c(ccc6cc7oc8ccccc8c7cc65)c4)cc32)cc1]

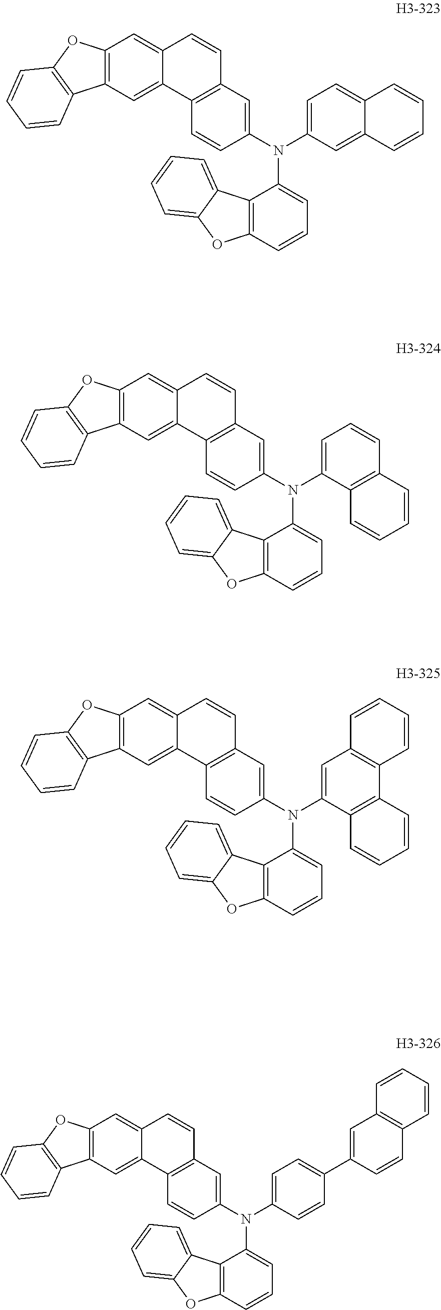 [SMILES: c1ccc2cc(N(c3ccc4c(ccc5cc6oc7ccccc7c6cc54)c3)c3cccc4oc5ccccc5c34)ccc2c1 ; c1ccc2c(N(c3ccc4c(ccc5cc6oc7ccccc7c6cc54)c3)c3cccc4oc5ccccc5c34)cccc2c1 ; c1ccc2c(c1)cc(N(c1ccc3c(ccc4cc5oc6ccccc6c5cc43)c1)c1cccc3oc4ccccc4c13)c1ccccc12 ; c1ccc2cc(-c3ccc(N(c4ccc5c(ccc6cc7oc8ccccc8c7cc65)c4)c4cccc5oc6ccccc6c45)cc3)ccc2c1]

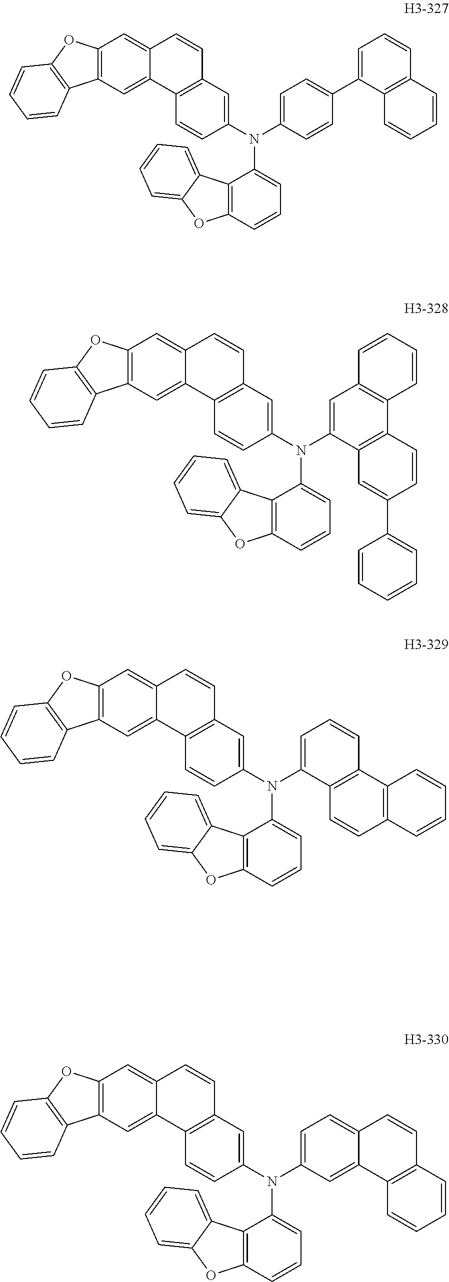 [SMILES: c1ccc2c(-c3ccc(N(c4ccc5c(ccc6cc7oc8ccccc8c7cc65)c4)c4cccc5oc6ccccc6c45)cc3)cccc2c1 ; c1ccc(-c2ccc3c(c2)c(N(c2ccc4c(ccc5cc6oc7ccccc7c6cc54)c2)c2cccc4oc5ccccc5c24)cc2ccccc23)cc1 ; c1ccc2c(c1)ccc1c(N(c3ccc4c(ccc5cc6oc7ccccc7c6cc54)c3)c3cccc4oc5ccccc5c34)cccc12 ; c1ccc2c(c1)ccc1ccc(N(c3ccc4c(ccc5cc6oc7ccccc7c6cc54)c3)c3cccc4oc5ccccc5c34)cc12]

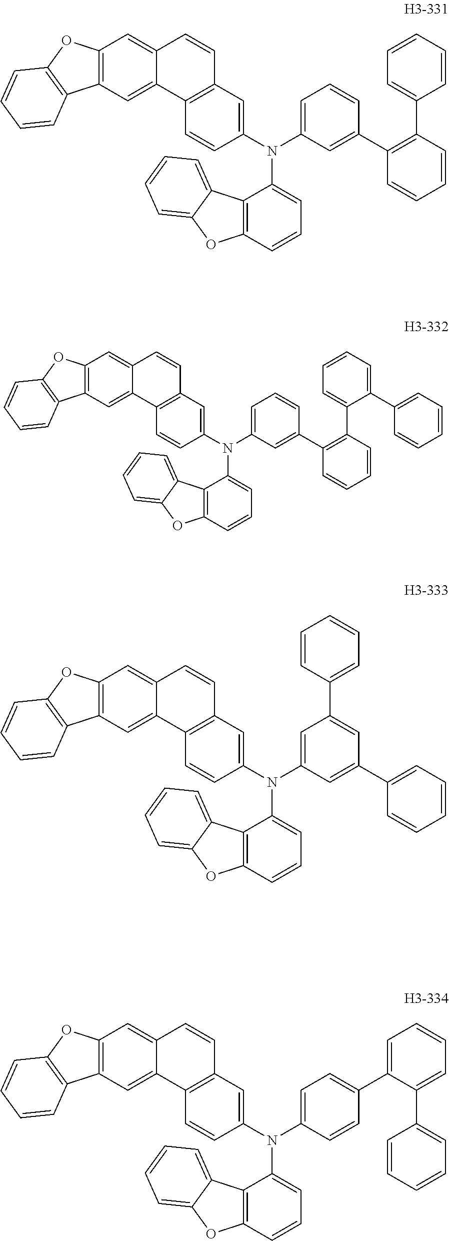 [SMILES: c1ccc(-c2ccccc2-c2cccc(N(c3ccc4c(ccc5cc6oc7ccccc7c6cc54)c3)c3cccc4oc5ccccc5c34)c2)cc1 ; c1ccc(-c2ccccc2-c2ccccc2-c2cccc(N(c3ccc4c(ccc5cc6oc7ccccc7c6cc54)c3)c3cccc4oc5ccccc5c34)c2)cc1 ; c1ccc(-c2cc(-c3ccccc3)cc(N(c3ccc4c(ccc5cc6oc7ccccc7c6cc54)c3)c3cccc4oc5ccccc5c34)c2)cc1 ; c1ccc(-c2ccccc2-c2ccc(N(c3ccc4c(ccc5cc6oc7ccccc7c6cc54)c3)c3cccc4oc5ccccc5c34)cc2)cc1]

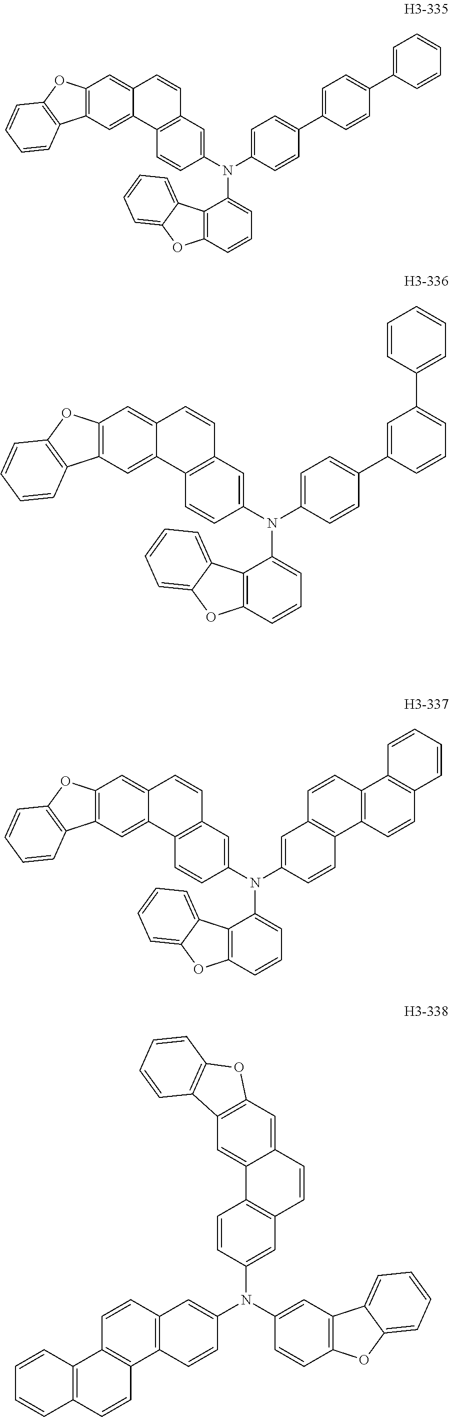 [SMILES: c1ccc(-c2ccc(-c3ccc(N(c4ccc5c(ccc6cc7oc8ccccc8c7cc65)c4)c4cccc5oc6ccccc6c45)cc3)cc2)cc1 ; c1ccc(-c2cccc(-c3ccc(N(c4ccc5c(ccc6cc7oc8ccccc8c7cc65)c4)c4cccc5oc6ccccc6c45)cc3)c2)cc1 ; c1ccc2c(c1)ccc1c3ccc(N(c4ccc5c(ccc6cc7oc8ccccc8c7cc65)c4)c4cccc5oc6ccccc6c45)cc3ccc21 ; c1ccc2c(c1)ccc1c3ccc(N(c4ccc5c(ccc6cc7oc8ccccc8c7cc65)c4)c4ccc5oc6ccccc6c5c4)cc3ccc21]

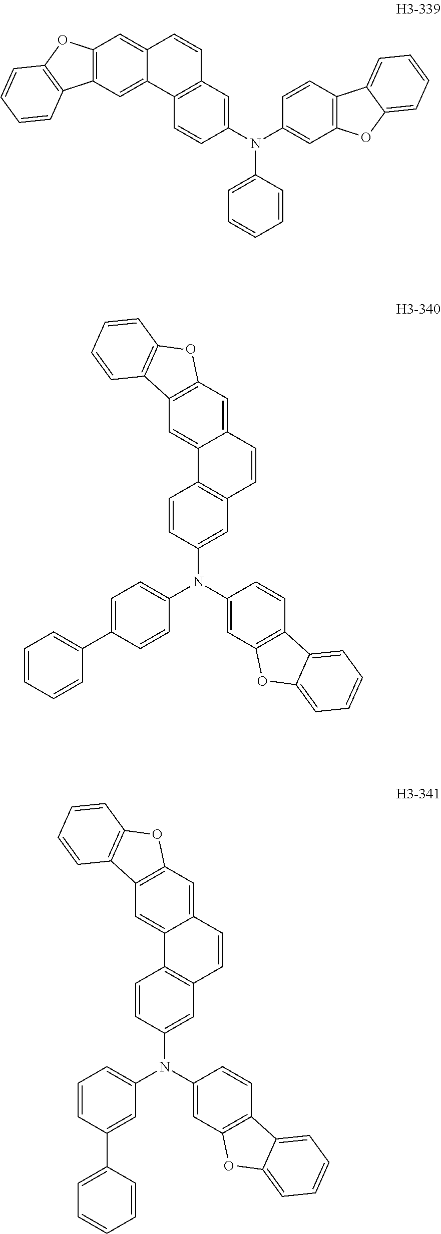 [SMILES: c1ccc(N(c2ccc3c(ccc4cc5oc6ccccc6c5cc43)c2)c2ccc3c(c2)oc2ccccc23)cc1 ; c1ccc(-c2ccc(N(c3ccc4c(ccc5cc6oc7ccccc7c6cc54)c3)c3ccc4c(c3)oc3ccccc34)cc2)cc1 ; c1ccc(-c2cccc(N(c3ccc4c(ccc5cc6oc7ccccc7c6cc54)c3)c3ccc4c(c3)oc3ccccc34)c2)cc1]

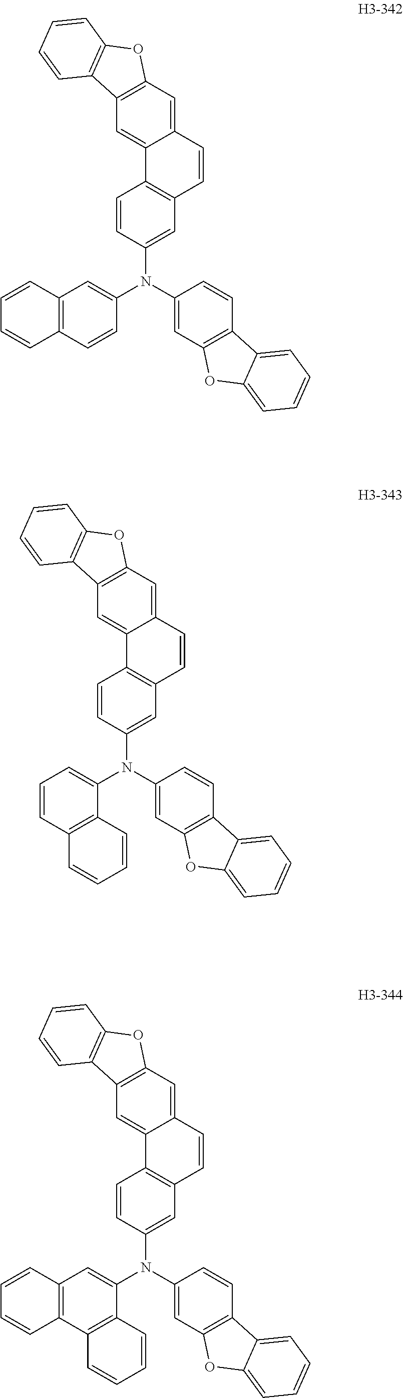 [SMILES: c1ccc2cc(N(c3ccc4c(ccc5cc6oc7ccccc7c6cc54)c3)c3ccc4c(c3)oc3ccccc34)ccc2c1 ; c1ccc2c(N(c3ccc4c(ccc5cc6oc7ccccc7c6cc54)c3)c3ccc4c(c3)oc3ccccc34)cccc2c1 ; c1ccc2c(c1)cc(N(c1ccc3c(ccc4cc5oc6ccccc6c5cc43)c1)c1ccc3c(c1)oc1ccccc13)c1ccccc12]

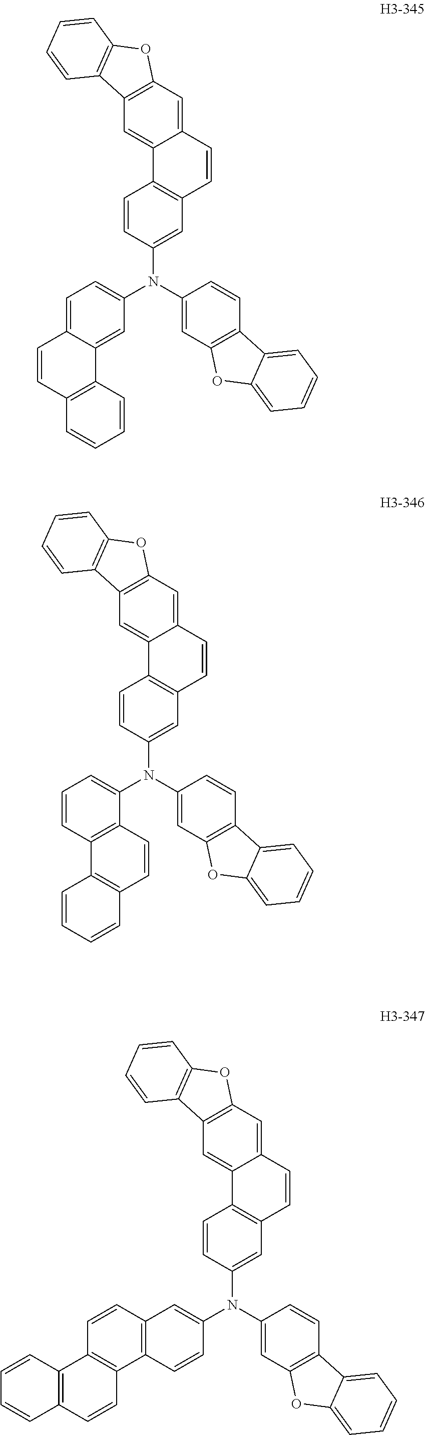 [SMILES: c1ccc2c(c1)ccc1ccc(N(c3ccc4c(ccc5cc6oc7ccccc7c6cc54)c3)c3ccc4c(c3)oc3ccccc34)cc12 ; c1ccc2c(c1)ccc1c(N(c3ccc4c(ccc5cc6oc7ccccc7c6cc54)c3)c3ccc4c(c3)oc3ccccc34)cccc12 ; c1ccc2c(c1)ccc1c3ccc(N(c4ccc5c(ccc6cc7oc8ccccc8c7cc65)c4)c4ccc5c(c4)oc4ccccc45)cc3ccc21]

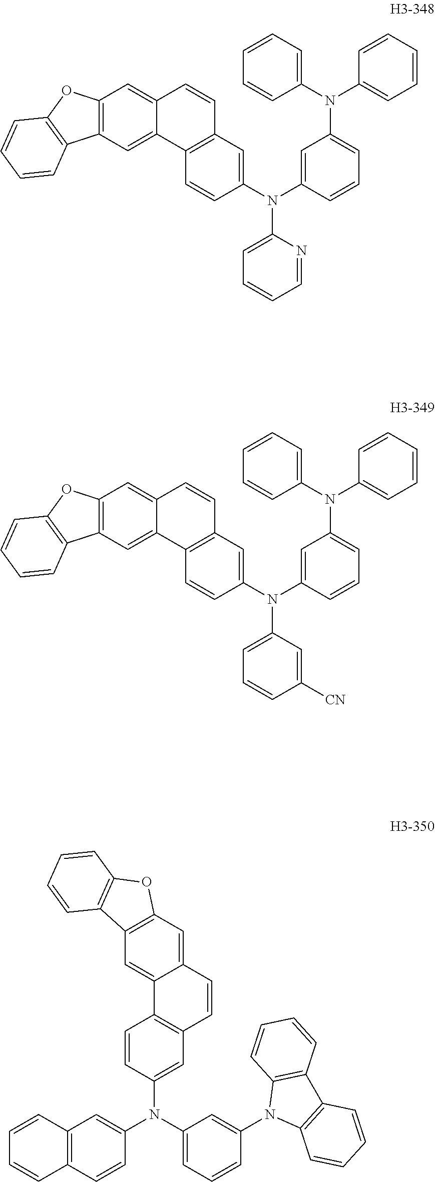 [SMILES: c1ccc(N(c2ccccc2)c2cccc(N(c3ccc4c(ccc5cc6oc7ccccc7c6cc54)c3)c3ccccn3)c2)cc1 ; N#Cc1cccc(N(c2cccc(N(c3ccccc3)c3ccccc3)c2)c2ccc3c(ccc4cc5oc6ccccc6c5cc43)c2)c1 ; c1cc(N(c2ccc3ccccc3c2)c2ccc3c(ccc4cc5oc6ccccc6c5cc43)c2)cc(-n2c3ccccc3c3ccccc32)c1]

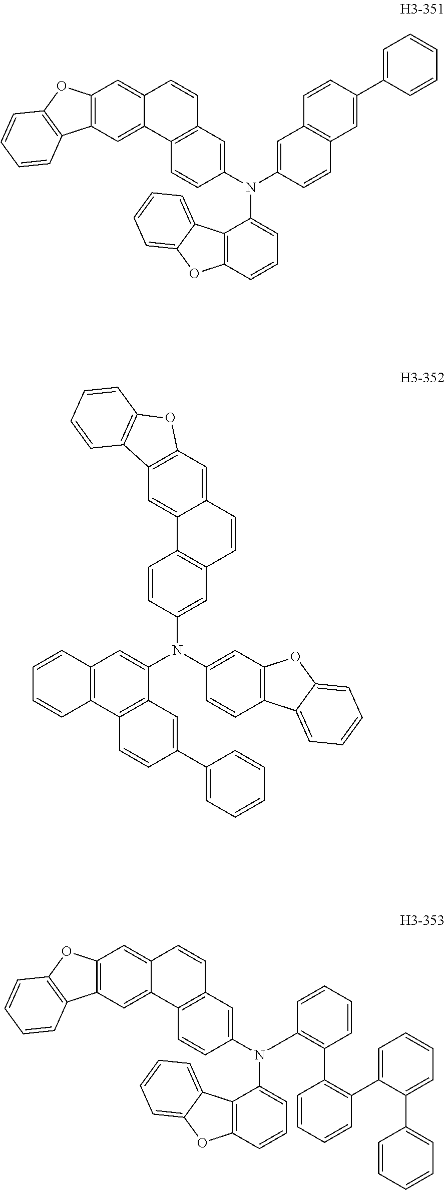 [SMILES: c1ccc(-c2ccc3cc(N(c4ccc5c(ccc6cc7oc8ccccc8c7cc65)c4)c4cccc5oc6ccccc6c45)ccc3c2)cc1 ; c1ccc(-c2ccc3c(c2)c(N(c2ccc4c(ccc5cc6oc7ccccc7c6cc54)c2)c2ccc4c(c2)oc2ccccc24)cc2ccccc23)cc1 ; c1ccc(-c2ccccc2-c2ccccc2-c2ccccc2N(c2ccc3c(ccc4cc5oc6ccccc6c5cc43)c2)c2cccc3oc4ccccc4c23)cc1]

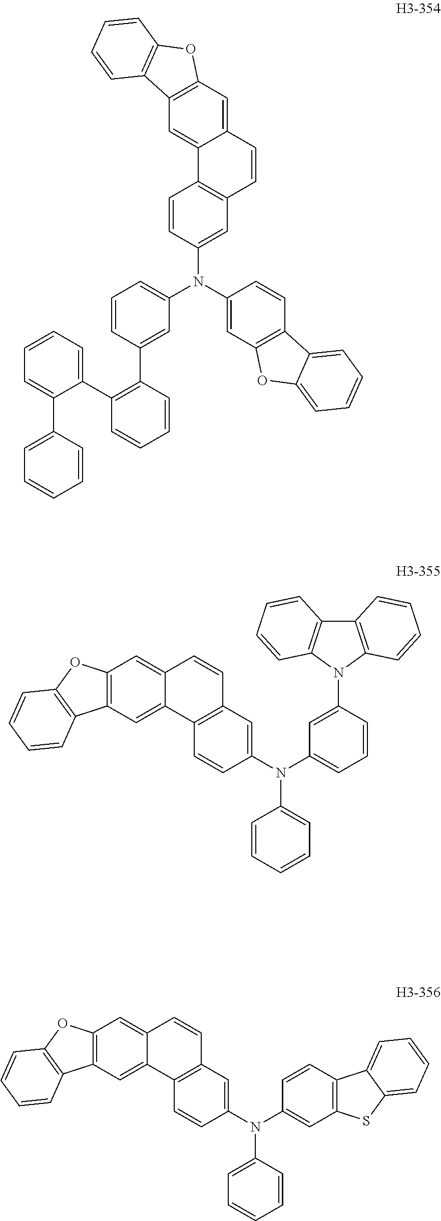 [SMILES: c1ccc(-c2ccccc2-c2ccccc2-c2cccc(N(c3ccc4c(ccc5cc6oc7ccccc7c6cc54)c3)c3ccc4c(c3)oc3ccccc34)c2)cc1 ; c1ccc(N(c2cccc(-n3c4ccccc4c4ccccc43)c2)c2ccc3c(ccc4cc5oc6ccccc6c5cc43)c2)cc1 ; c1ccc(N(c2ccc3c(ccc4cc5oc6ccccc6c5cc43)c2)c2ccc3c(c2)sc2ccccc23)cc1]

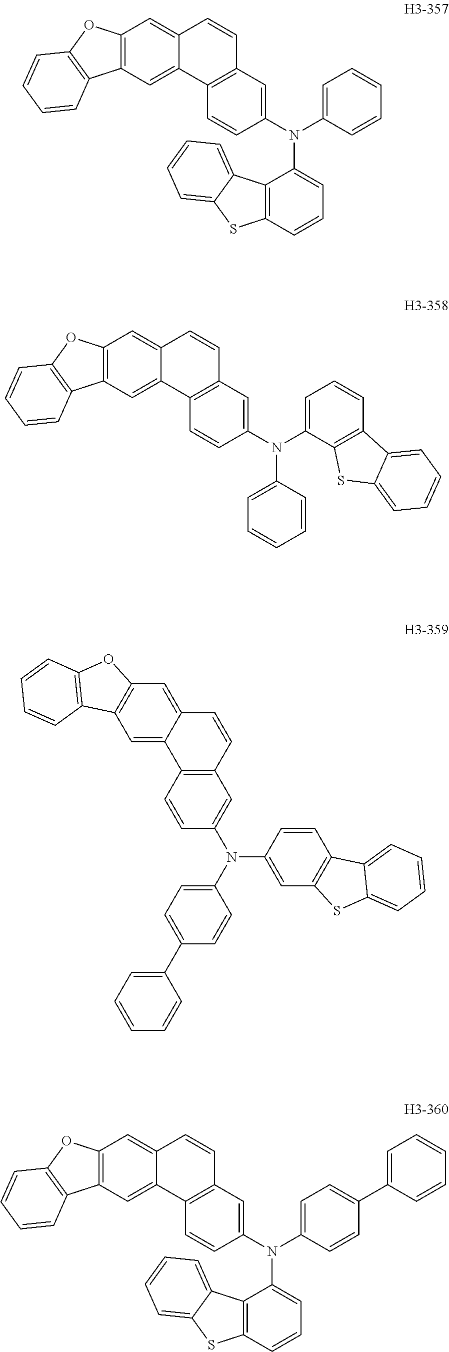 [SMILES: c1ccc(N(c2ccc3c(ccc4cc5oc6ccccc6c5cc43)c2)c2cccc3sc4ccccc4c23)cc1 ; c1ccc(N(c2ccc3c(ccc4cc5oc6ccccc6c5cc43)c2)c2cccc3c2sc2ccccc23)cc1 ; c1ccc(-c2ccc(N(c3ccc4c(ccc5cc6oc7ccccc7c6cc54)c3)c3ccc4c(c3)sc3ccccc34)cc2)cc1 ; c1ccc(-c2ccc(N(c3ccc4c(ccc5cc6oc7ccccc7c6cc54)c3)c3cccc4sc5ccccc5c34)cc2)cc1]

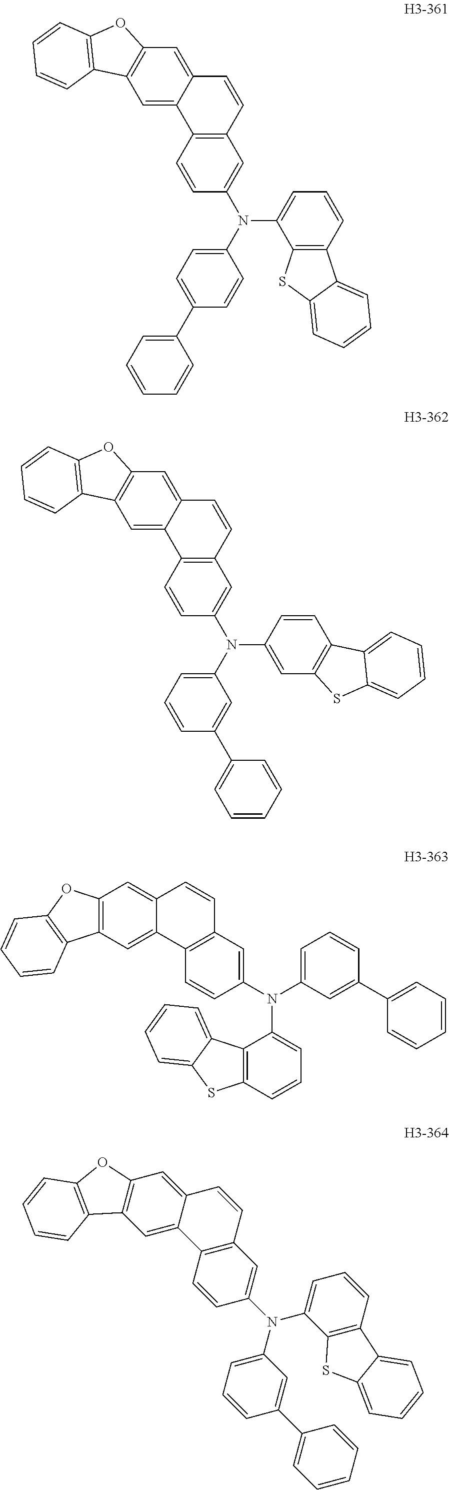 [SMILES: c1ccc(-c2ccc(N(c3ccc4c(ccc5cc6oc7ccccc7c6cc54)c3)c3cccc4c3sc3ccccc34)cc2)cc1 ; c1ccc(-c2cccc(N(c3ccc4c(ccc5cc6oc7ccccc7c6cc54)c3)c3ccc4c(c3)sc3ccccc34)c2)cc1 ; c1ccc(-c2cccc(N(c3ccc4c(ccc5cc6oc7ccccc7c6cc54)c3)c3cccc4sc5ccccc5c34)c2)cc1 ; c1ccc(-c2cccc(N(c3ccc4c(ccc5cc6oc7ccccc7c6cc54)c3)c3cccc4c3sc3ccccc34)c2)cc1]

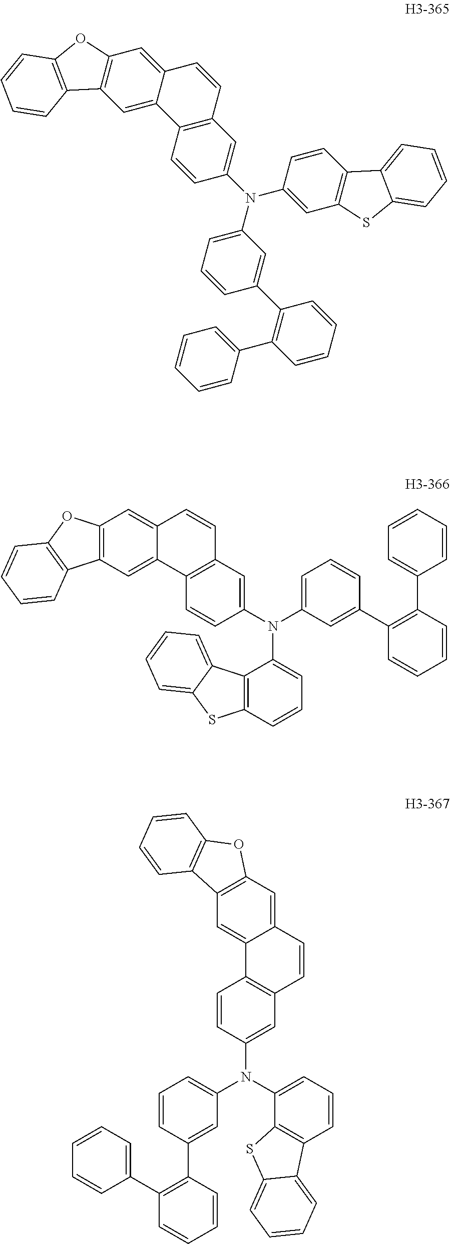 [SMILES: c1ccc(-c2ccccc2-c2cccc(N(c3ccc4c(ccc5cc6oc7ccccc7c6cc54)c3)c3ccc4c(c3)sc3ccccc34)c2)cc1 ; c1ccc(-c2ccccc2-c2cccc(N(c3ccc4c(ccc5cc6oc7ccccc7c6cc54)c3)c3cccc4sc5ccccc5c34)c2)cc1 ; c1ccc(-c2ccccc2-c2cccc(N(c3ccc4c(ccc5cc6oc7ccccc7c6cc54)c3)c3cccc4c3sc3ccccc34)c2)cc1]

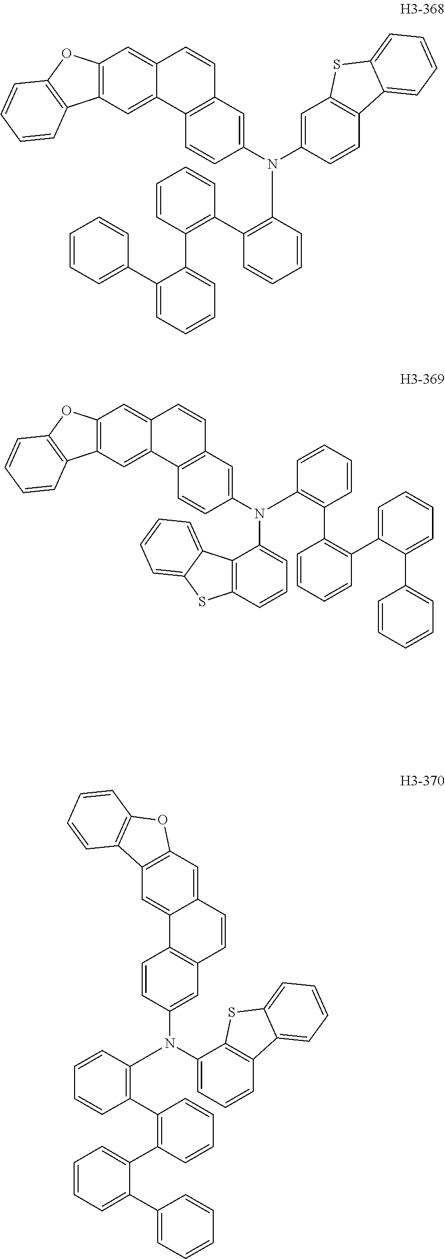 [SMILES: c1ccc(-c2ccccc2-c2ccccc2-c2ccccc2N(c2ccc3c(ccc4cc5oc6ccccc6c5cc43)c2)c2ccc3c(c2)sc2ccccc23)cc1 ; c1ccc(-c2ccccc2-c2ccccc2-c2ccccc2N(c2ccc3c(ccc4cc5oc6ccccc6c5cc43)c2)c2cccc3sc4ccccc4c23)cc1 ; c1ccc(-c2ccccc2-c2ccccc2-c2ccccc2N(c2ccc3c(ccc4cc5oc6ccccc6c5cc43)c2)c2cccc3c2sc2ccccc23)cc1]

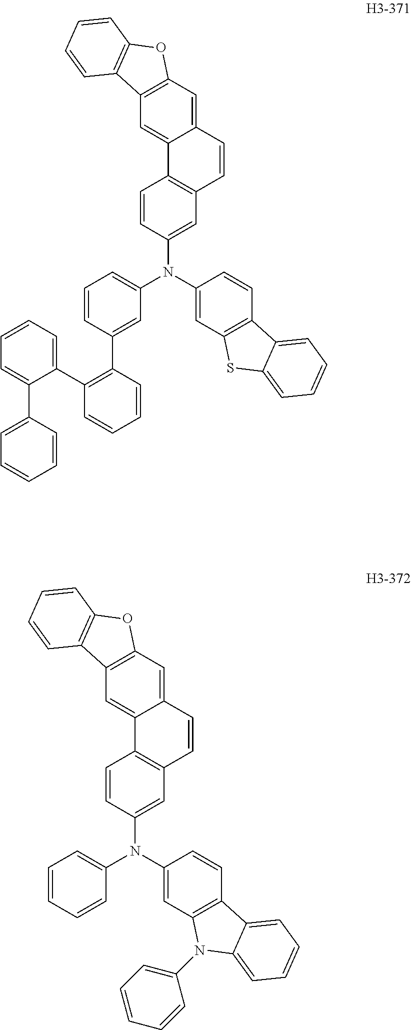 [SMILES: c1ccc(-c2ccccc2-c2ccccc2-c2cccc(N(c3ccc4c(ccc5cc6oc7ccccc7c6cc54)c3)c3ccc4c(c3)sc3ccccc34)c2)cc1 ; c1ccc(N(c2ccc3c(ccc4cc5oc6ccccc6c5cc43)c2)c2ccc3c4ccccc4n(-c4ccccc4)c3c2)cc1]

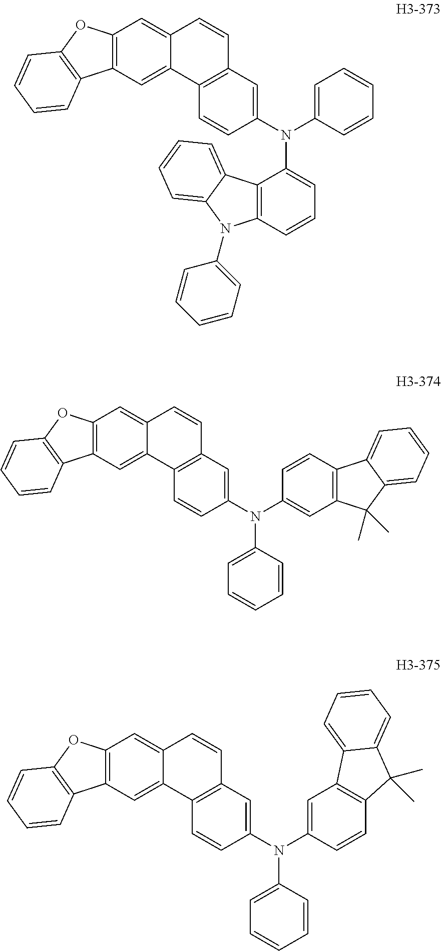 [SMILES: c1ccc(N(c2ccc3c(ccc4cc5oc6ccccc6c5cc43)c2)c2cccc3c2c2ccccc2n3-c2ccccc2)cc1 ; CC1(C)c2ccccc2-c2ccc(N(c3ccccc3)c3ccc4c(ccc5cc6oc7ccccc7c6cc54)c3)cc21 ; CC1(C)c2ccccc2-c2cc(N(c3ccccc3)c3ccc4c(ccc5cc6oc7ccccc7c6cc54)c3)ccc21]

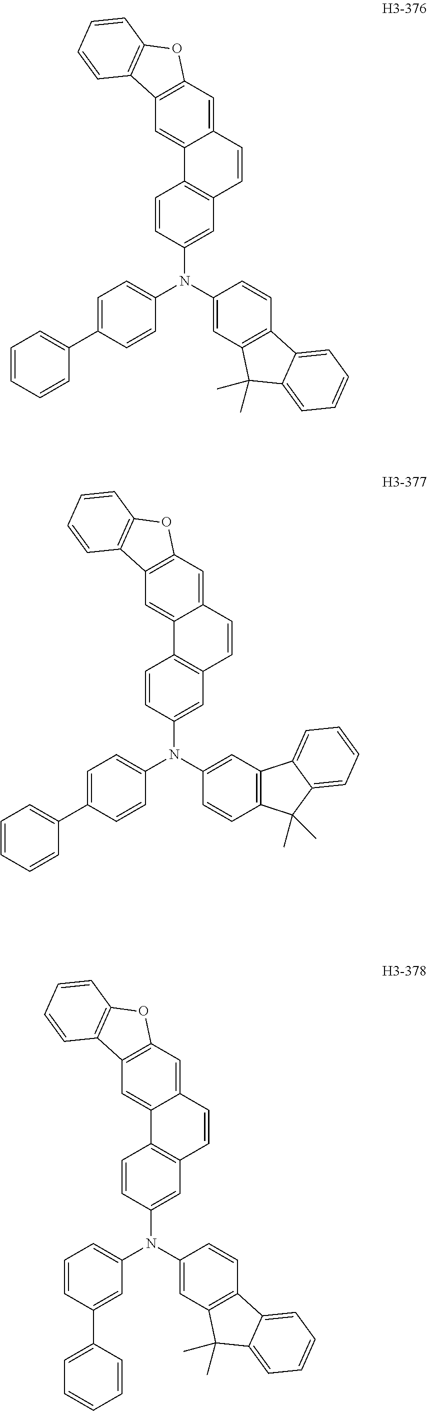 [SMILES: CC1(C)c2ccccc2-c2ccc(N(c3ccc(-c4ccccc4)cc3)c3ccc4c(ccc5cc6oc7ccccc7c6cc54)c3)cc21 ; CC1(C)c2ccccc2-c2cc(N(c3ccc(-c4ccccc4)cc3)c3ccc4c(ccc5cc6oc7ccccc7c6cc54)c3)ccc21 ; CC1(C)c2ccccc2-c2ccc(N(c3cccc(-c4ccccc4)c3)c3ccc4c(ccc5cc6oc7ccccc7c6cc54)c3)cc21]

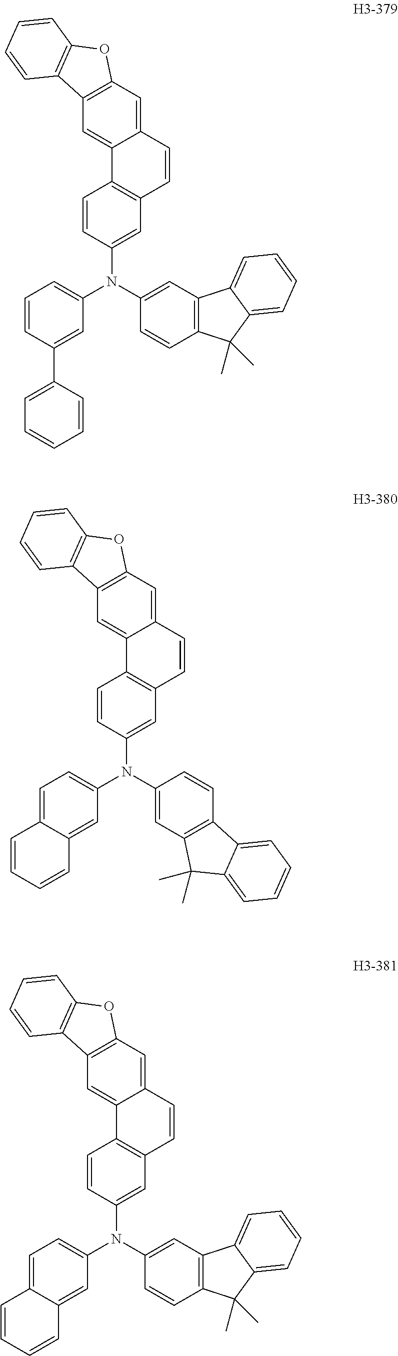 [SMILES: CC1(C)c2ccccc2-c2cc(N(c3cccc(-c4ccccc4)c3)c3ccc4c(ccc5cc6oc7ccccc7c6cc54)c3)ccc21 ; CC1(C)c2ccccc2-c2ccc(N(c3ccc4ccccc4c3)c3ccc4c(ccc5cc6oc7ccccc7c6cc54)c3)cc21 ; CC1(C)c2ccccc2-c2cc(N(c3ccc4ccccc4c3)c3ccc4c(ccc5cc6oc7ccccc7c6cc54)c3)ccc21]

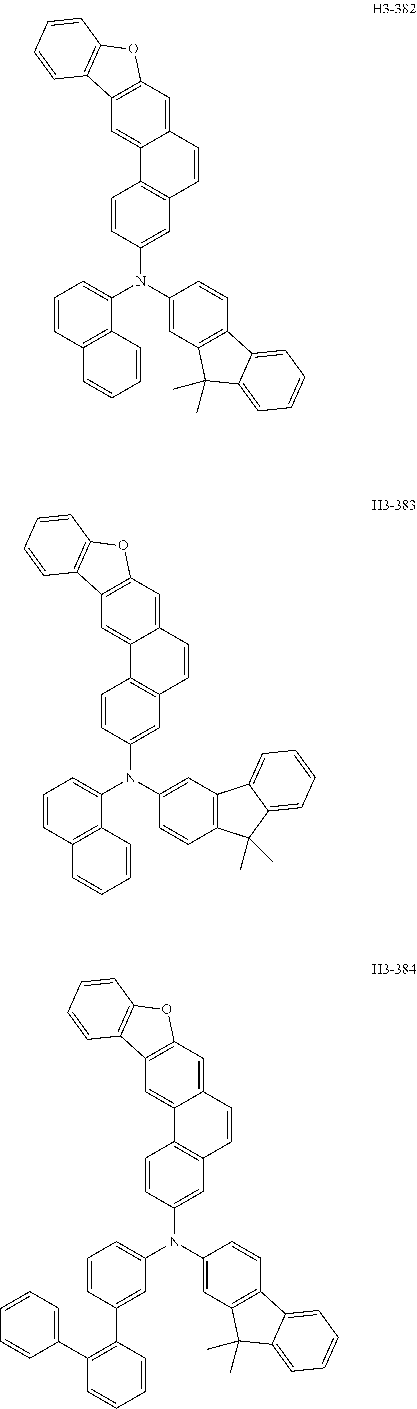 [SMILES: CC1(C)c2ccccc2-c2ccc(N(c3ccc4c(ccc5cc6oc7ccccc7c6cc54)c3)c3cccc4ccccc34)cc21 ; CC1(C)c2ccccc2-c2cc(N(c3ccc4c(ccc5cc6oc7ccccc7c6cc54)c3)c3cccc4ccccc34)ccc21 ; CC1(C)c2ccccc2-c2ccc(N(c3cccc(-c4ccccc4-c4ccccc4)c3)c3ccc4c(ccc5cc6oc7ccccc7c6cc54)c3)cc21]

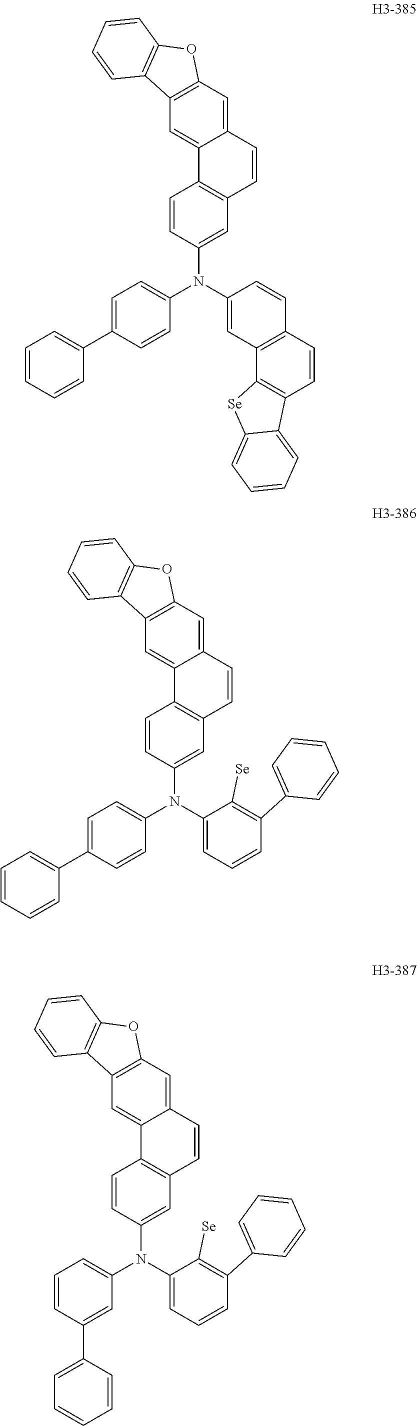

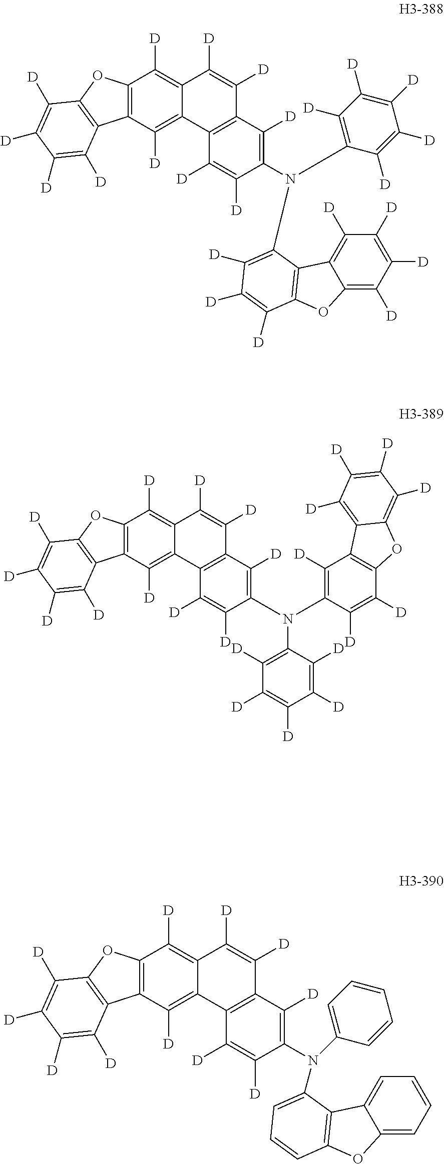

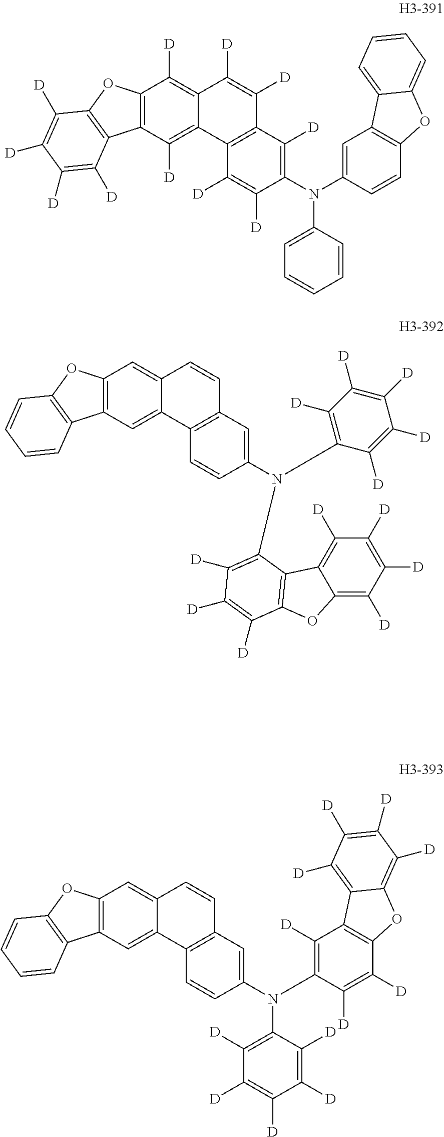 [SMILES: [2H]c1c([2H])c([2H])c2c(oc3c([2H])c4c([2H])c([2H])c5c([2H])c(N(c6ccccc6)c6ccc7oc8ccccc8c7c6)c([2H])c([2H])c5c4c([2H])c32)c1[2H] ; [2H]c1c([2H])c([2H])c(N(c2ccc3c(ccc4cc5oc6ccccc6c5cc43)c2)c2c([2H])c([2H])c([2H])c3oc4c([2H])c([2H])c([2H])c([2H])c4c23)c([2H])c1[2H] ; [2H]c1c([2H])c([2H])c(N(c2ccc3c(ccc4cc5oc6ccccc6c5cc43)c2)c2c([2H])c([2H])c3oc4c([2H])c([2H])c([2H])c([2H])c4c3c2[2H])c([2H])c1[2H]]

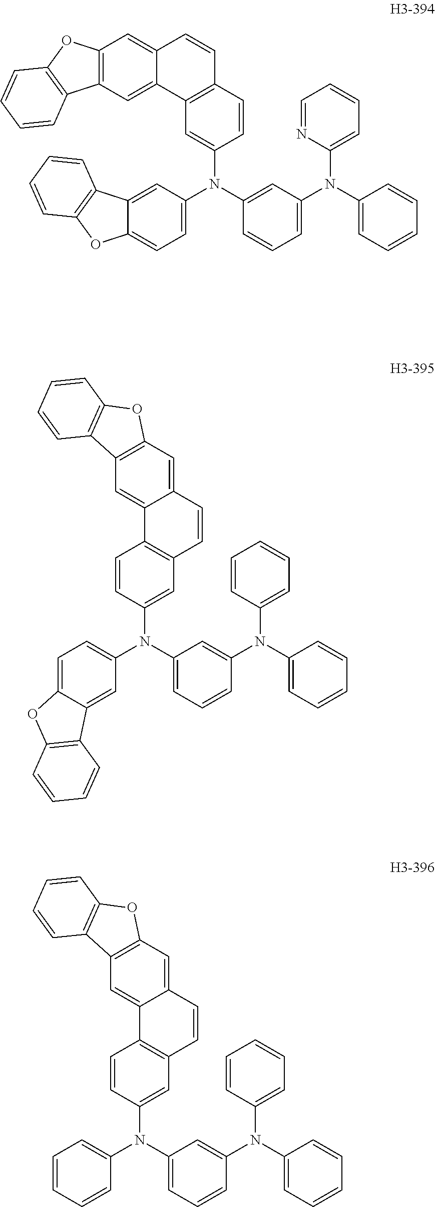 [SMILES: c1ccc(N(c2cccc(N(c3ccc4ccc5cc6oc7ccccc7c6cc5c4c3)c3ccc4oc5ccccc5c4c3)c2)c2ccccn2)cc1 ; c1ccc(N(c2ccccc2)c2cccc(N(c3ccc4c(ccc5cc6oc7ccccc7c6cc54)c3)c3ccc4oc5ccccc5c4c3)c2)cc1 ; c1ccc(N(c2ccccc2)c2cccc(N(c3ccccc3)c3ccc4c(ccc5cc6oc7ccccc7c6cc54)c3)c2)cc1]

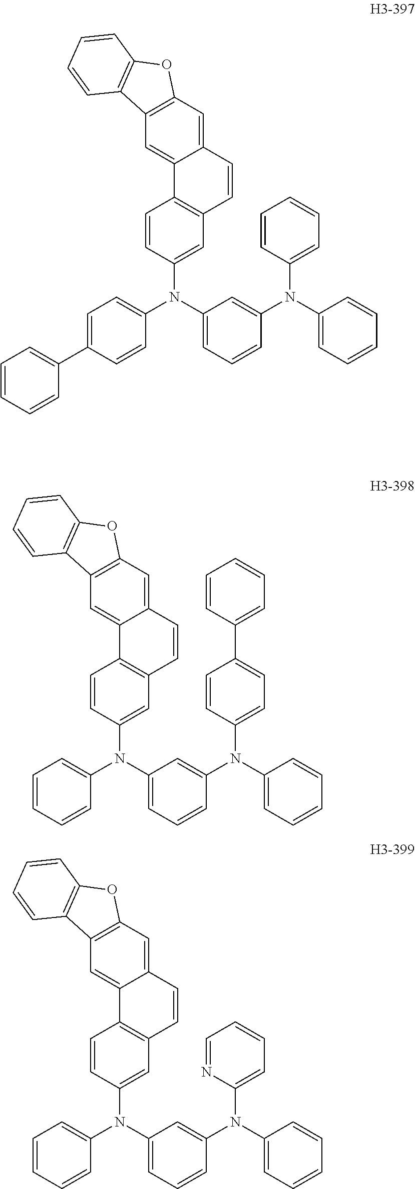

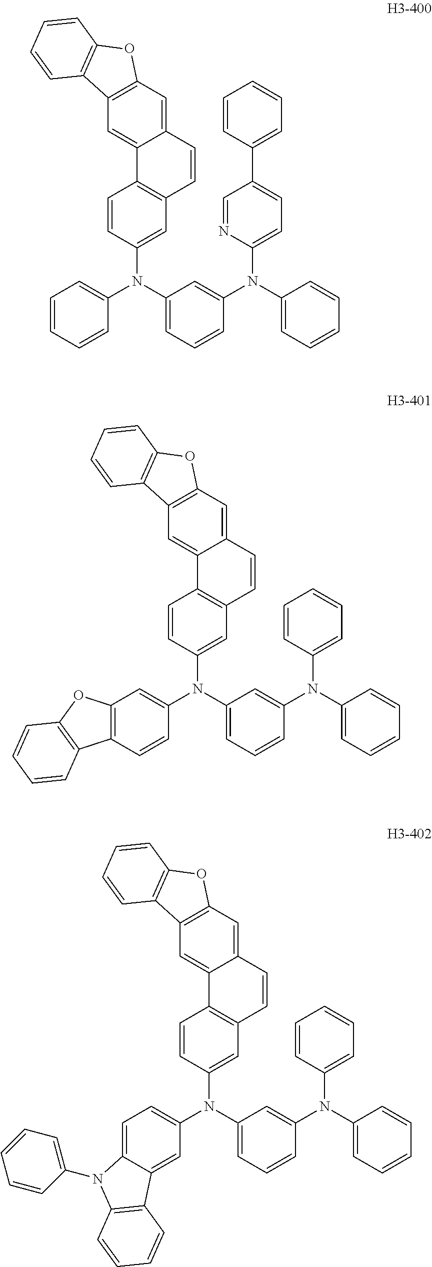 [SMILES: c1ccc(-c2ccc(N(c3ccccc3)c3cccc(N(c4ccccc4)c4ccc5c(ccc6cc7oc8ccccc8c7cc65)c4)c3)nc2)cc1 ; c1ccc(N(c2ccccc2)c2cccc(N(c3ccc4c(ccc5cc6oc7ccccc7c6cc54)c3)c3ccc4c(c3)oc3ccccc34)c2)cc1 ; c1ccc(N(c2ccccc2)c2cccc(N(c3ccc4c(ccc5cc6oc7ccccc7c6cc54)c3)c3ccc4c(c3)c3ccccc3n4-c3ccccc3)c2)cc1]

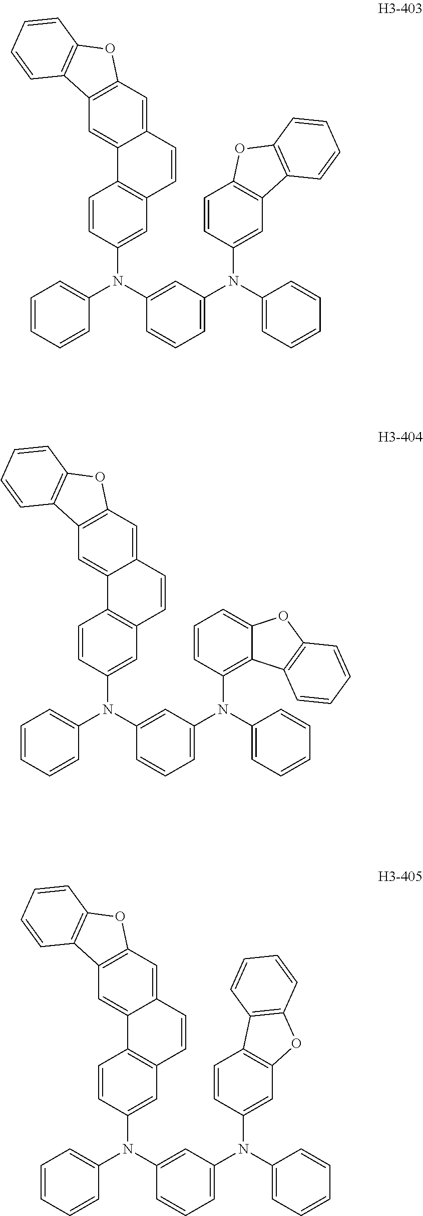 [SMILES: c1ccc(N(c2cccc(N(c3ccccc3)c3ccc4oc5ccccc5c4c3)c2)c2ccc3c(ccc4cc5oc6ccccc6c5cc43)c2)cc1 ; c1ccc(N(c2cccc(N(c3ccccc3)c3cccc4oc5ccccc5c34)c2)c2ccc3c(ccc4cc5oc6ccccc6c5cc43)c2)cc1 ; c1ccc(N(c2cccc(N(c3ccccc3)c3ccc4c(c3)oc3ccccc34)c2)c2ccc3c(ccc4cc5oc6ccccc6c5cc43)c2)cc1]

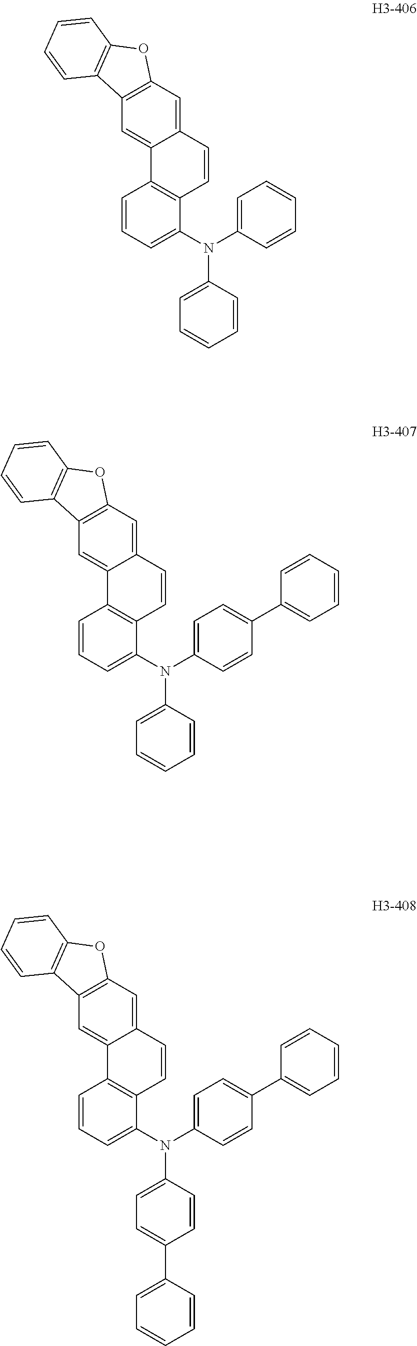 [SMILES: c1ccc(N(c2ccccc2)c2cccc3c2ccc2cc4oc5ccccc5c4cc23)cc1 ; c1ccc(-c2ccc(N(c3ccccc3)c3cccc4c3ccc3cc5oc6ccccc6c5cc34)cc2)cc1 ; c1ccc(-c2ccc(N(c3ccc(-c4ccccc4)cc3)c3cccc4c3ccc3cc5oc6ccccc6c5cc34)cc2)cc1]

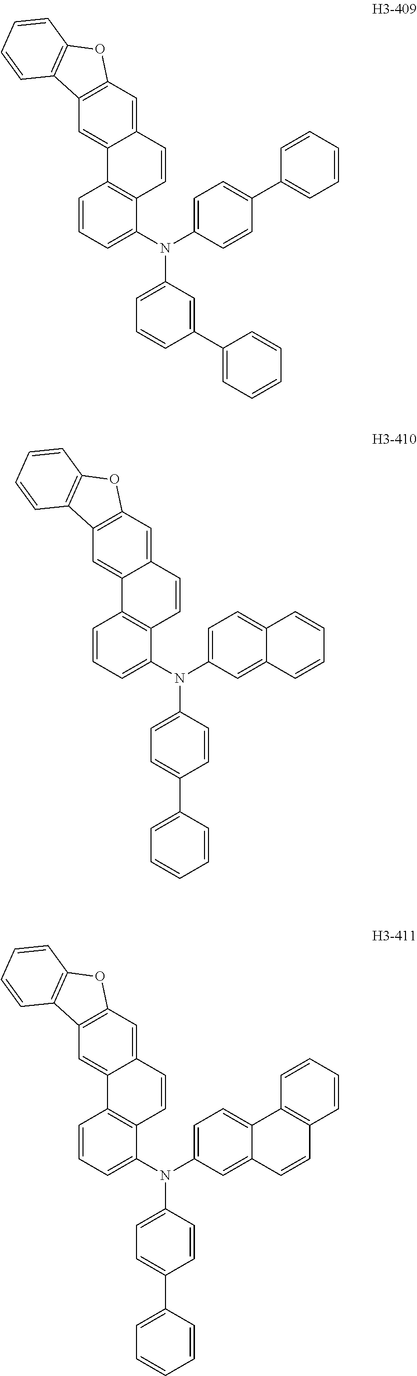 [SMILES: c1ccc(-c2ccc(N(c3cccc(-c4ccccc4)c3)c3cccc4c3ccc3cc5oc6ccccc6c5cc34)cc2)cc1 ; c1ccc(-c2ccc(N(c3ccc4ccccc4c3)c3cccc4c3ccc3cc5oc6ccccc6c5cc34)cc2)cc1 ; c1ccc(-c2ccc(N(c3ccc4c(ccc5ccccc54)c3)c3cccc4c3ccc3cc5oc6ccccc6c5cc34)cc2)cc1]

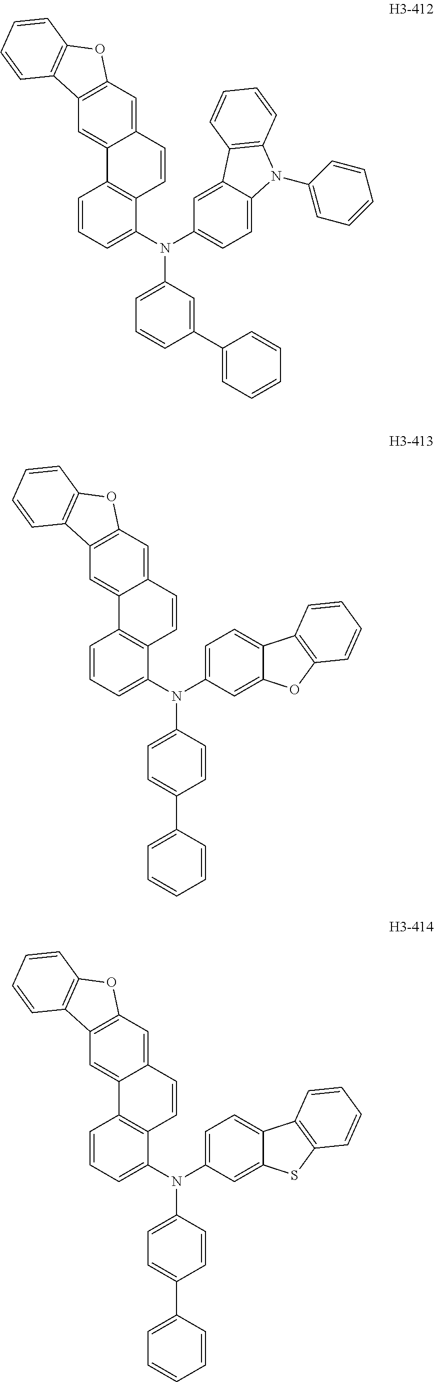 [SMILES: c1ccc(-c2cccc(N(c3ccc4c(c3)c3ccccc3n4-c3ccccc3)c3cccc4c3ccc3cc5oc6ccccc6c5cc34)c2)cc1 ; c1ccc(-c2ccc(N(c3ccc4c(c3)oc3ccccc34)c3cccc4c3ccc3cc5oc6ccccc6c5cc34)cc2)cc1 ; c1ccc(-c2ccc(N(c3ccc4c(c3)sc3ccccc34)c3cccc4c3ccc3cc5oc6ccccc6c5cc34)cc2)cc1]

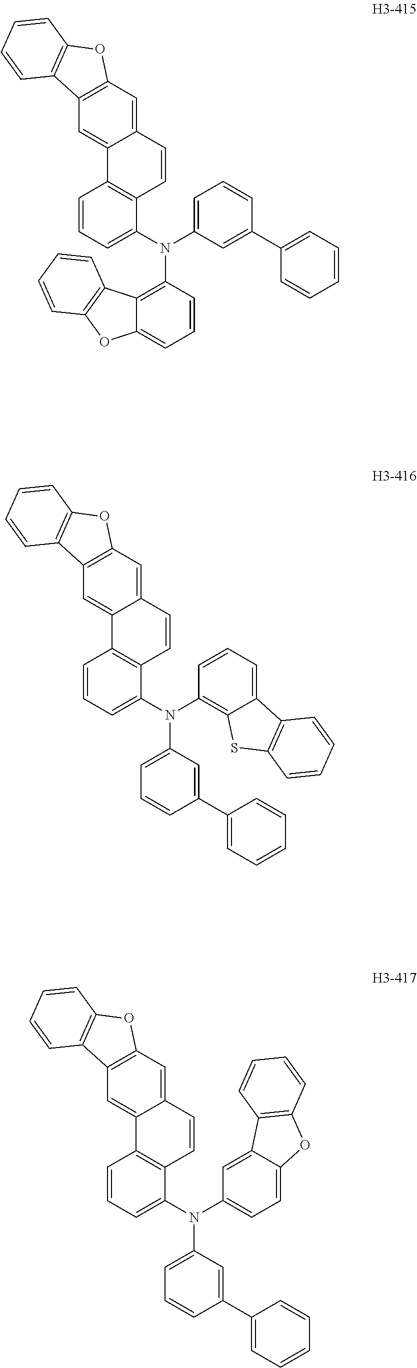 [SMILES: c1ccc(-c2cccc(N(c3cccc4c3ccc3cc5oc6ccccc6c5cc34)c3cccc4oc5ccccc5c34)c2)cc1 ; c1ccc(-c2cccc(N(c3cccc4c3ccc3cc5oc6ccccc6c5cc34)c3cccc4c3sc3ccccc34)c2)cc1 ; c1ccc(-c2cccc(N(c3ccc4oc5ccccc5c4c3)c3cccc4c3ccc3cc5oc6ccccc6c5cc34)c2)cc1]

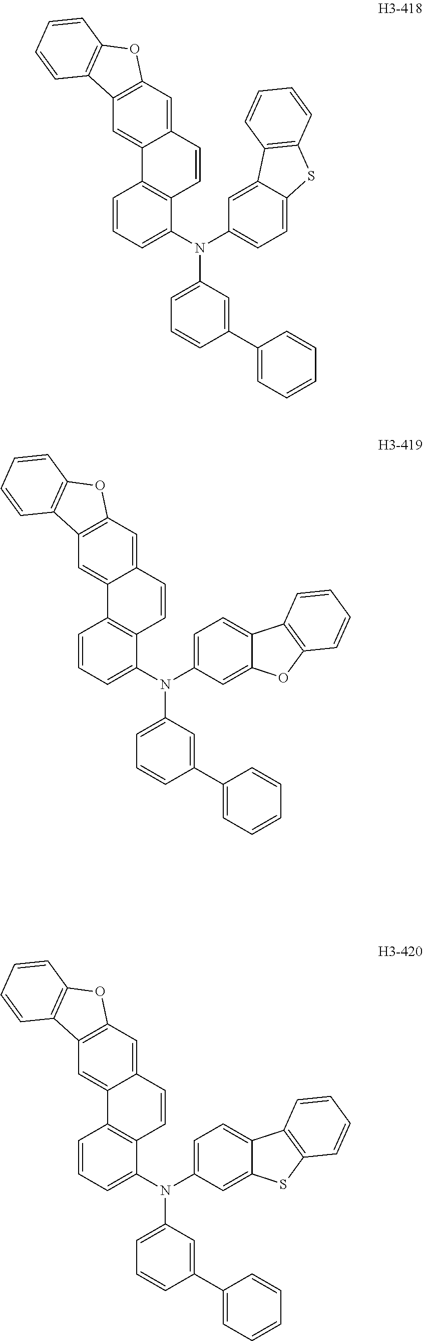 [SMILES: c1ccc(-c2cccc(N(c3ccc4sc5ccccc5c4c3)c3cccc4c3ccc3cc5oc6ccccc6c5cc34)c2)cc1 ; c1ccc(-c2cccc(N(c3ccc4c(c3)oc3ccccc34)c3cccc4c3ccc3cc5oc6ccccc6c5cc34)c2)cc1 ; c1ccc(-c2cccc(N(c3ccc4c(c3)sc3ccccc34)c3cccc4c3ccc3cc5oc6ccccc6c5cc34)c2)cc1]

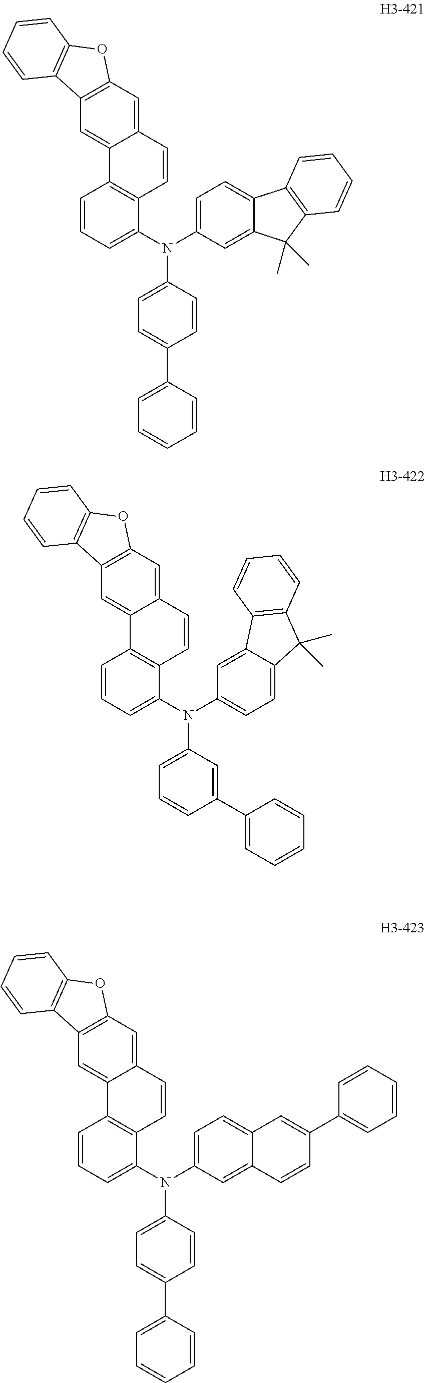 [SMILES: CC1(C)c2ccccc2-c2ccc(N(c3ccc(-c4ccccc4)cc3)c3cccc4c3ccc3cc5oc6ccccc6c5cc34)cc21 ; CC1(C)c2ccccc2-c2cc(N(c3cccc(-c4ccccc4)c3)c3cccc4c3ccc3cc5oc6ccccc6c5cc34)ccc21 ; c1ccc(-c2ccc(N(c3ccc4cc(-c5ccccc5)ccc4c3)c3cccc4c3ccc3cc5oc6ccccc6c5cc34)cc2)cc1]

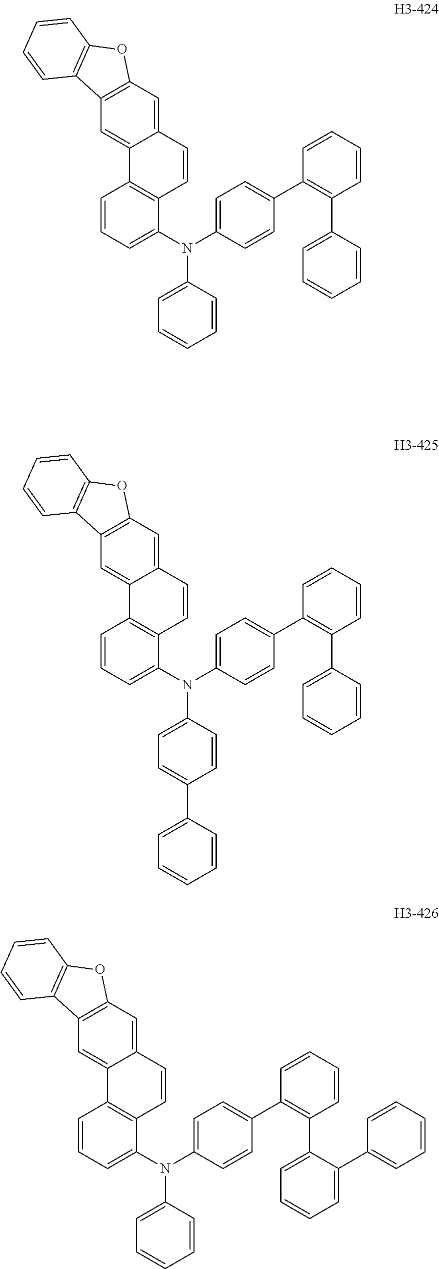 [SMILES: c1ccc(-c2ccccc2-c2ccc(N(c3ccccc3)c3cccc4c3ccc3cc5oc6ccccc6c5cc34)cc2)cc1 ; c1ccc(-c2ccc(N(c3ccc(-c4ccccc4-c4ccccc4)cc3)c3cccc4c3ccc3cc5oc6ccccc6c5cc34)cc2)cc1 ; c1ccc(-c2ccccc2-c2ccccc2-c2ccc(N(c3ccccc3)c3cccc4c3ccc3cc5oc6ccccc6c5cc34)cc2)cc1]

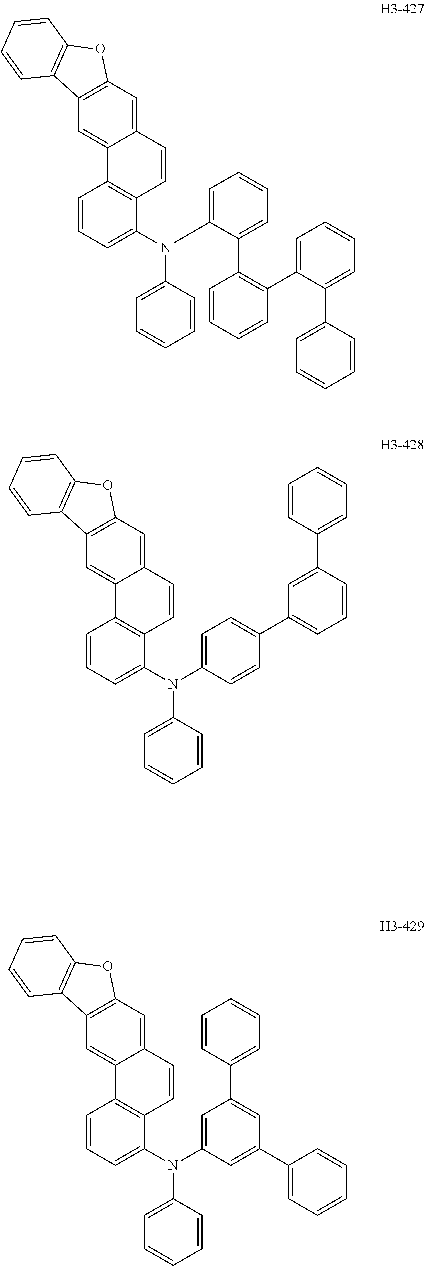 [SMILES: c1ccc(-c2ccccc2-c2ccccc2-c2ccccc2N(c2ccccc2)c2cccc3c2ccc2cc4oc5ccccc5c4cc23)cc1 ; c1ccc(-c2cccc(-c3ccc(N(c4ccccc4)c4cccc5c4ccc4cc6oc7ccccc7c6cc45)cc3)c2)cc1 ; c1ccc(-c2cc(-c3ccccc3)cc(N(c3ccccc3)c3cccc4c3ccc3cc5oc6ccccc6c5cc34)c2)cc1]

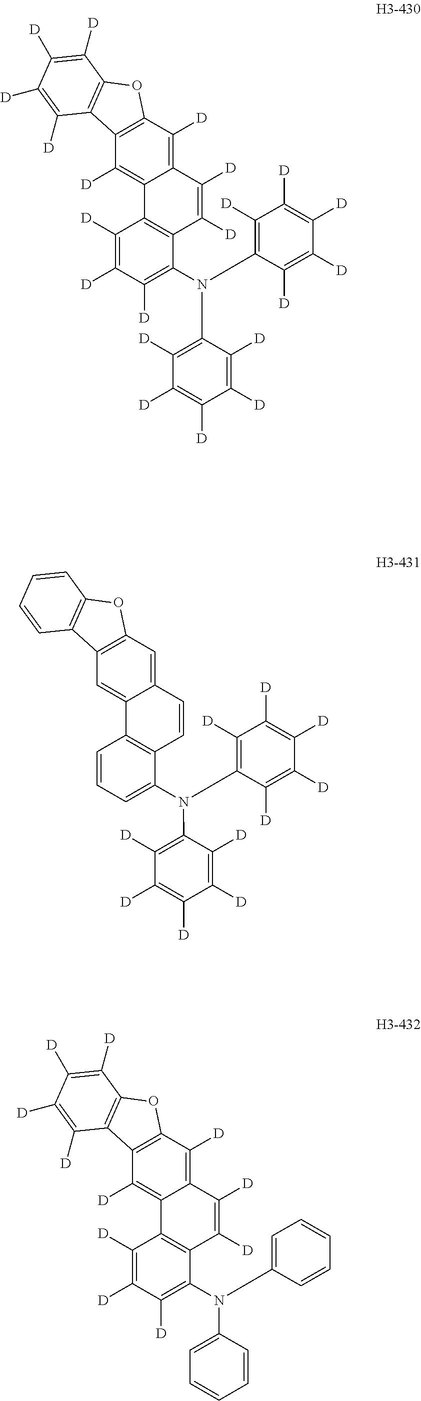 [SMILES: [2H]c1c([2H])c([2H])c(N(c2c([2H])c([2H])c([2H])c([2H])c2[2H])c2c([2H])c([2H])c([2H])c3c2c([2H])c([2H])c2c([2H])c4oc5c([2H])c([2H])c([2H])c([2H])c5c4c([2H])c23)c([2H])c1[2H] ; [2H]c1c([2H])c([2H])c(N(c2c([2H])c([2H])c([2H])c([2H])c2[2H])c2cccc3c2ccc2cc4oc5ccccc5c4cc23)c([2H])c1[2H] ; [2H]c1c([2H])c([2H])c2c(oc3c([2H])c4c([2H])c([2H])c5c(N(c6ccccc6)c6ccccc6)c([2H])c([2H])c([2H])c5c4c([2H])c32)c1[2H]]

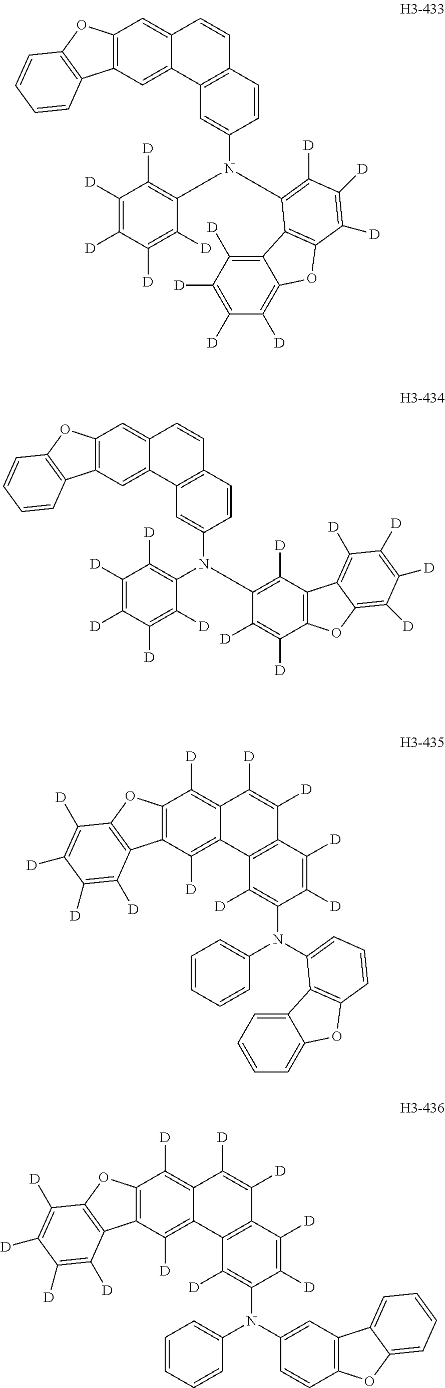

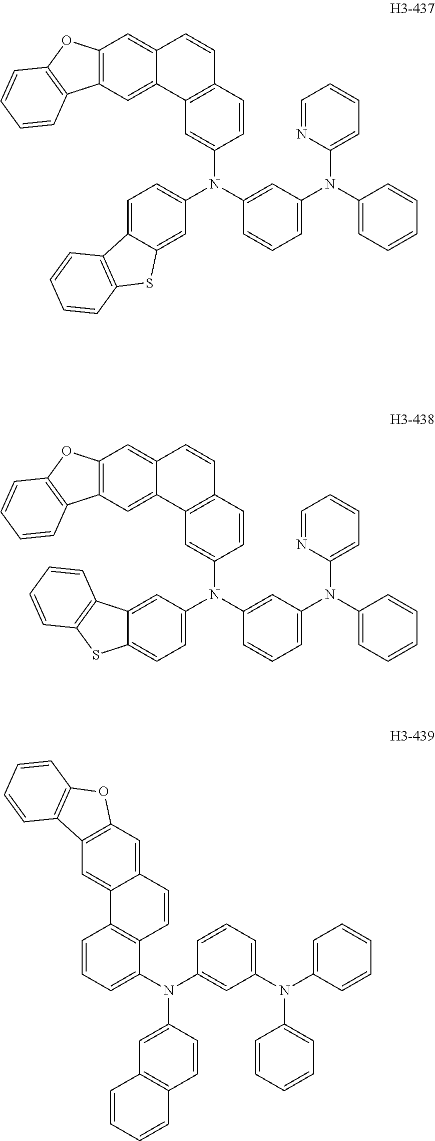 [SMILES: c1ccc(N(c2cccc(N(c3ccc4c(c3)sc3ccccc34)c3ccc4ccc5cc6oc7ccccc7c6cc5c4c3)c2)c2ccccn2)cc1 ; c1ccc(N(c2cccc(N(c3ccc4ccc5cc6oc7ccccc7c6cc5c4c3)c3ccc4sc5ccccc5c4c3)c2)c2ccccn2)cc1 ; c1ccc(N(c2ccccc2)c2cccc(N(c3ccc4ccccc4c3)c3cccc4c3ccc3cc5oc6ccccc6c5cc34)c2)cc1]

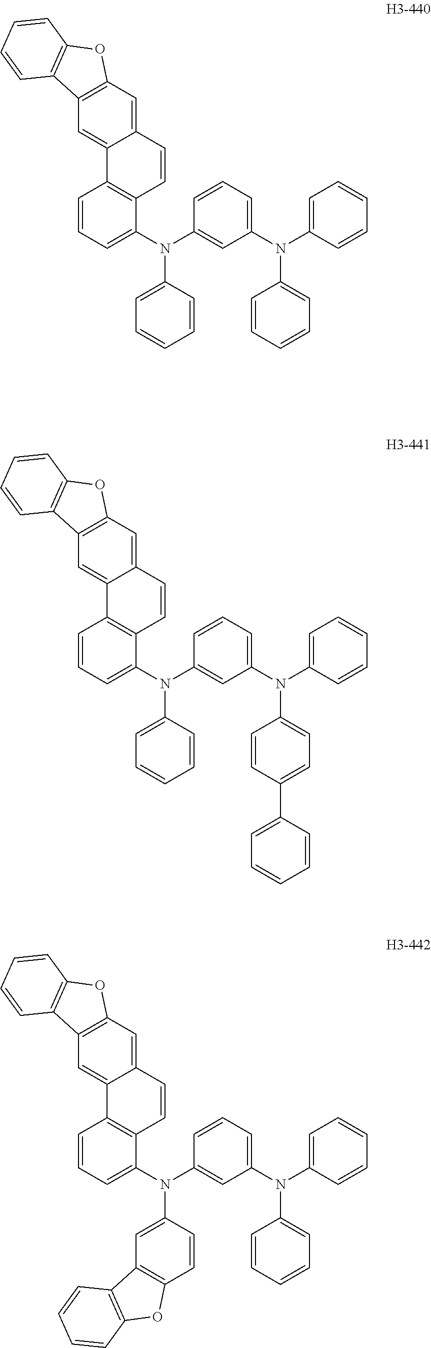 [SMILES: c1ccc(N(c2ccccc2)c2cccc(N(c3ccccc3)c3cccc4c3ccc3cc5oc6ccccc6c5cc34)c2)cc1 ; c1ccc(-c2ccc(N(c3ccccc3)c3cccc(N(c4ccccc4)c4cccc5c4ccc4cc6oc7ccccc7c6cc45)c3)cc2)cc1 ; c1ccc(N(c2ccccc2)c2cccc(N(c3ccc4oc5ccccc5c4c3)c3cccc4c3ccc3cc5oc6ccccc6c5cc34)c2)cc1]

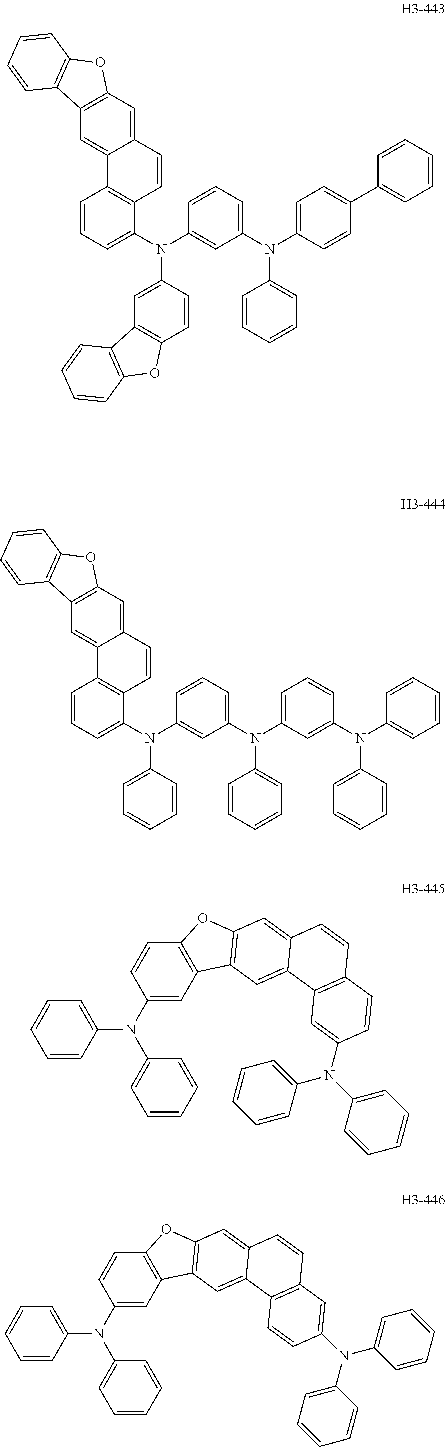 [SMILES: c1ccc(-c2ccc(N(c3ccccc3)c3cccc(N(c4ccc5oc6ccccc6c5c4)c4cccc5c4ccc4cc6oc7ccccc7c6cc45)c3)cc2)cc1 ; c1ccc(N(c2ccccc2)c2cccc(N(c3ccccc3)c3cccc(N(c4ccccc4)c4cccc5c4ccc4cc6oc7ccccc7c6cc45)c3)c2)cc1 ; c1ccc(N(c2ccccc2)c2ccc3ccc4cc5oc6ccc(N(c7ccccc7)c7ccccc7)cc6c5cc4c3c2)cc1 ; c1ccc(N(c2ccccc2)c2ccc3c(ccc4cc5oc6ccc(N(c7ccccc7)c7ccccc7)cc6c5cc43)c2)cc1]

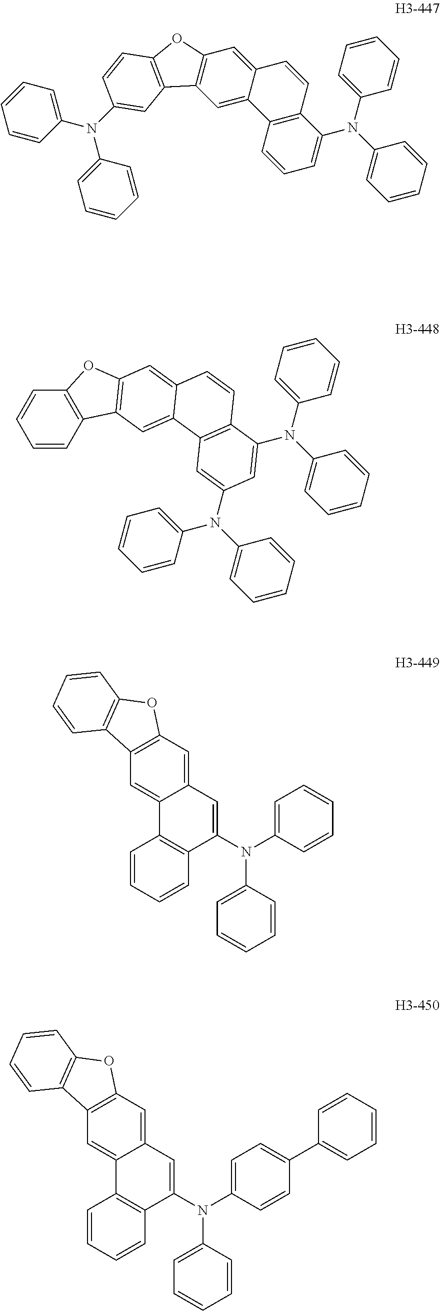 [SMILES: c1ccc(N(c2ccccc2)c2ccc3oc4cc5ccc6c(N(c7ccccc7)c7ccccc7)cccc6c5cc4c3c2)cc1 ; c1ccc(N(c2ccccc2)c2cc(N(c3ccccc3)c3ccccc3)c3ccc4cc5oc6ccccc6c5cc4c3c2)cc1 ; c1ccc(N(c2ccccc2)c2cc3cc4oc5ccccc5c4cc3c3ccccc23)cc1 ; c1ccc(-c2ccc(N(c3ccccc3)c3cc4cc5oc6ccccc6c5cc4c4ccccc34)cc2)cc1]

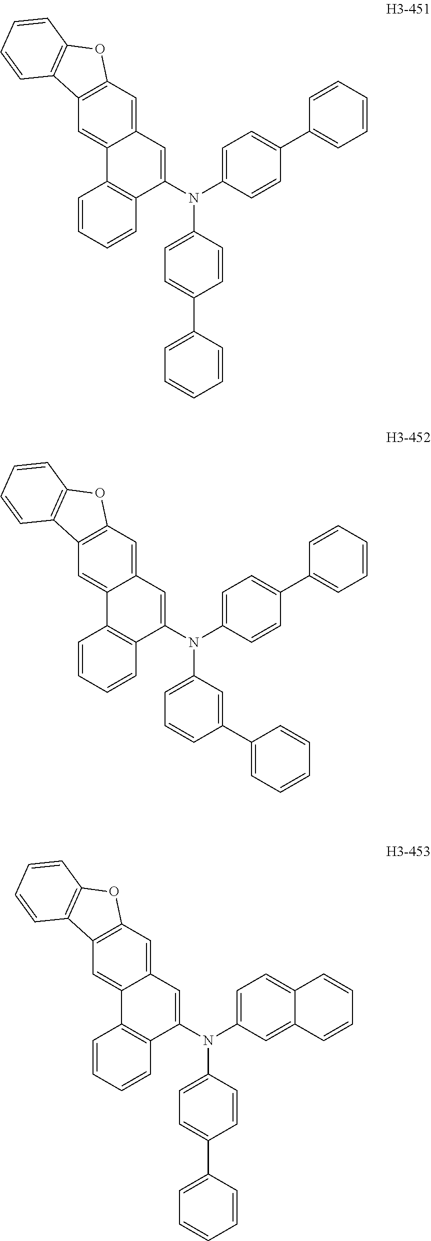 [SMILES: c1ccc(-c2ccc(N(c3ccc(-c4ccccc4)cc3)c3cc4cc5oc6ccccc6c5cc4c4ccccc34)cc2)cc1 ; c1ccc(-c2ccc(N(c3cccc(-c4ccccc4)c3)c3cc4cc5oc6ccccc6c5cc4c4ccccc34)cc2)cc1 ; c1ccc(-c2ccc(N(c3ccc4ccccc4c3)c3cc4cc5oc6ccccc6c5cc4c4ccccc34)cc2)cc1]

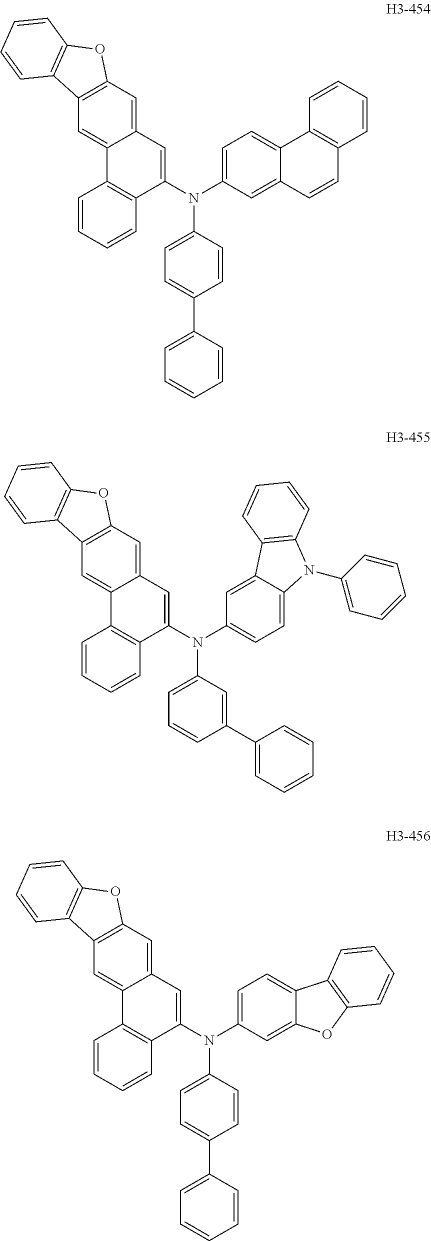 [SMILES: c1ccc(-c2ccc(N(c3ccc4c(ccc5ccccc54)c3)c3cc4cc5oc6ccccc6c5cc4c4ccccc34)cc2)cc1 ; c1ccc(-c2cccc(N(c3ccc4c(c3)c3ccccc3n4-c3ccccc3)c3cc4cc5oc6ccccc6c5cc4c4ccccc34)c2)cc1 ; c1ccc(-c2ccc(N(c3ccc4c(c3)oc3ccccc34)c3cc4cc5oc6ccccc6c5cc4c4ccccc34)cc2)cc1]

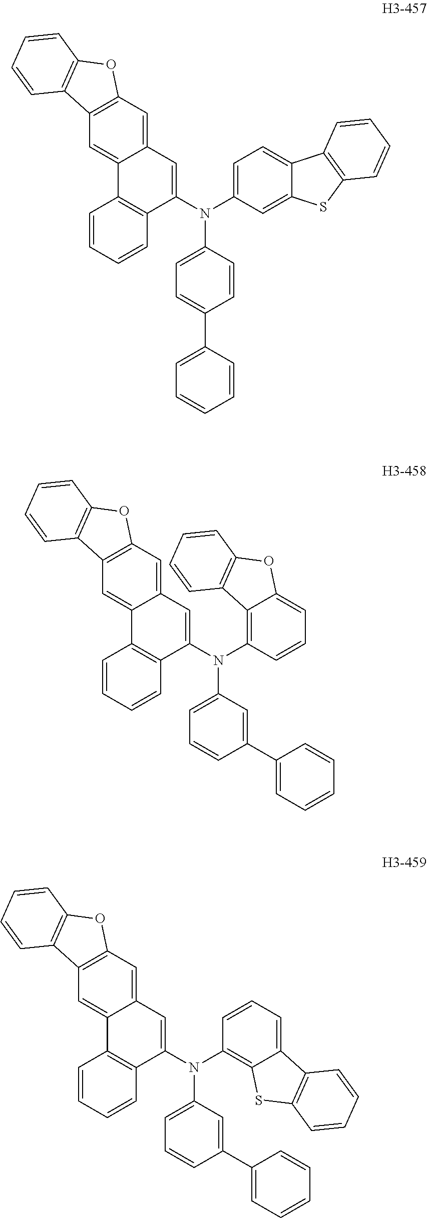 [SMILES: c1ccc(-c2ccc(N(c3ccc4c(c3)sc3ccccc34)c3cc4cc5oc6ccccc6c5cc4c4ccccc34)cc2)cc1 ; c1ccc(-c2cccc(N(c3cc4cc5oc6ccccc6c5cc4c4ccccc34)c3cccc4oc5ccccc5c34)c2)cc1 ; c1ccc(-c2cccc(N(c3cc4cc5oc6ccccc6c5cc4c4ccccc34)c3cccc4c3sc3ccccc34)c2)cc1]

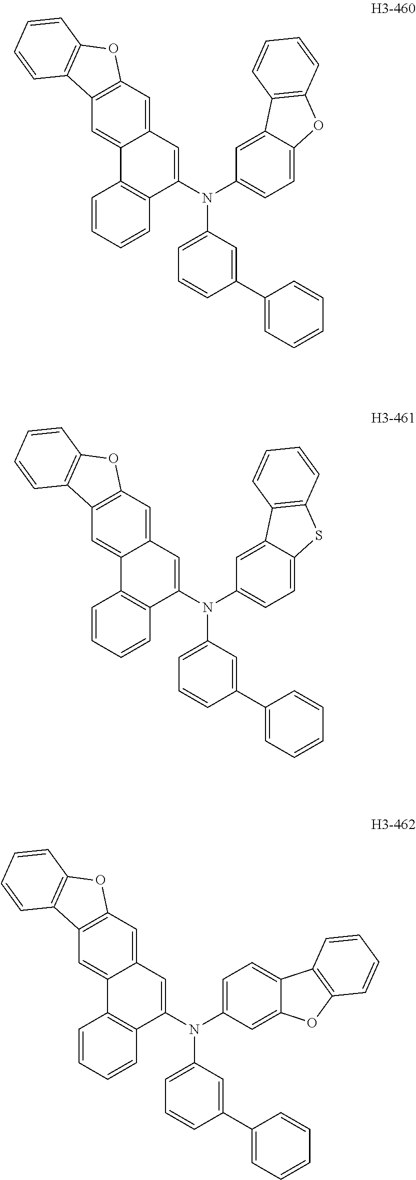 [SMILES: c1ccc(-c2cccc(N(c3ccc4oc5ccccc5c4c3)c3cc4cc5oc6ccccc6c5cc4c4ccccc34)c2)cc1 ; c1ccc(-c2cccc(N(c3ccc4sc5ccccc5c4c3)c3cc4cc5oc6ccccc6c5cc4c4ccccc34)c2)cc1 ; c1ccc(-c2cccc(N(c3ccc4c(c3)oc3ccccc34)c3cc4cc5oc6ccccc6c5cc4c4ccccc34)c2)cc1]

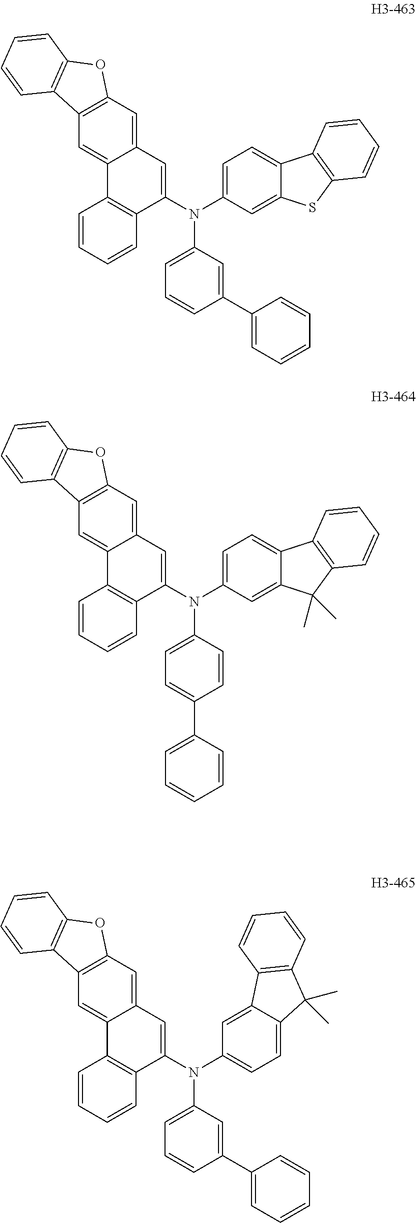 [SMILES: c1ccc(-c2cccc(N(c3ccc4c(c3)sc3ccccc34)c3cc4cc5oc6ccccc6c5cc4c4ccccc34)c2)cc1 ; CC1(C)c2ccccc2-c2ccc(N(c3ccc(-c4ccccc4)cc3)c3cc4cc5oc6ccccc6c5cc4c4ccccc34)cc21 ; CC1(C)c2ccccc2-c2cc(N(c3cccc(-c4ccccc4)c3)c3cc4cc5oc6ccccc6c5cc4c4ccccc34)ccc21]

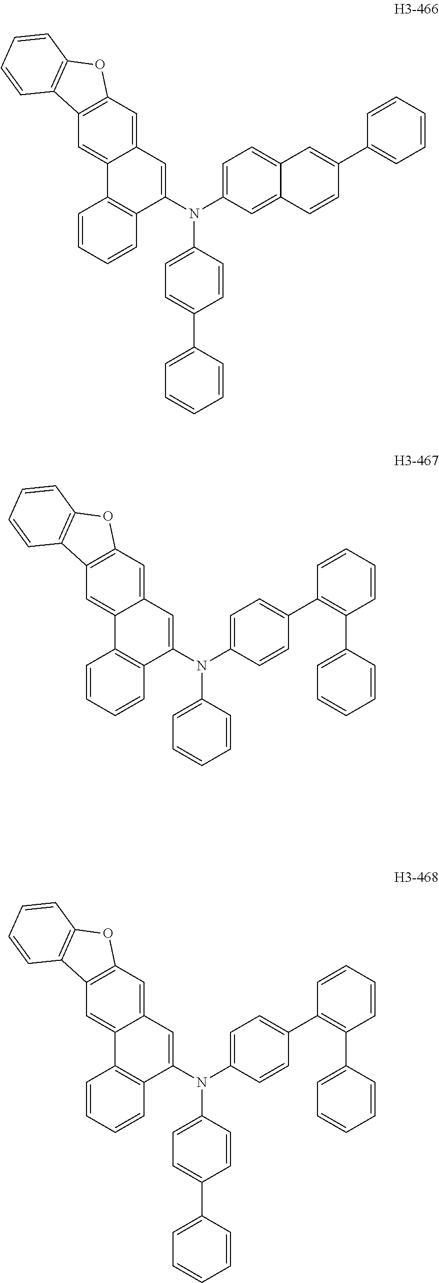 [SMILES: c1ccc(-c2ccc(N(c3ccc4cc(-c5ccccc5)ccc4c3)c3cc4cc5oc6ccccc6c5cc4c4ccccc34)cc2)cc1 ; c1ccc(-c2ccccc2-c2ccc(N(c3ccccc3)c3cc4cc5oc6ccccc6c5cc4c4ccccc34)cc2)cc1 ; c1ccc(-c2ccc(N(c3ccc(-c4ccccc4-c4ccccc4)cc3)c3cc4cc5oc6ccccc6c5cc4c4ccccc34)cc2)cc1]

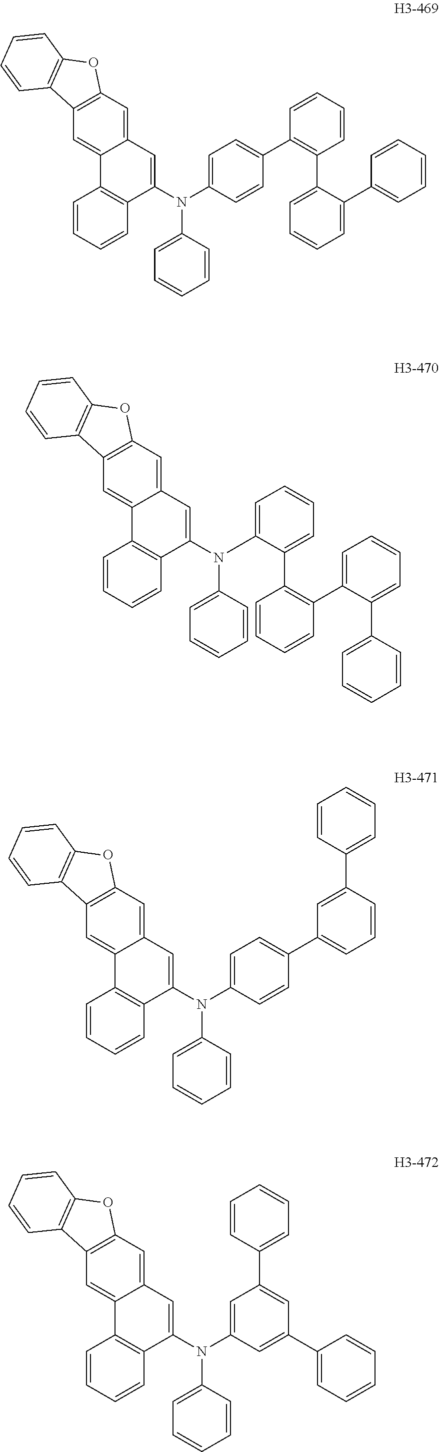 [SMILES: c1ccc(-c2ccccc2-c2ccccc2-c2ccc(N(c3ccccc3)c3cc4cc5oc6ccccc6c5cc4c4ccccc34)cc2)cc1 ; c1ccc(-c2ccccc2-c2ccccc2-c2ccccc2N(c2ccccc2)c2cc3cc4oc5ccccc5c4cc3c3ccccc23)cc1 ; c1ccc(-c2cccc(-c3ccc(N(c4ccccc4)c4cc5cc6oc7ccccc7c6cc5c5ccccc45)cc3)c2)cc1 ; c1ccc(-c2cc(-c3ccccc3)cc(N(c3ccccc3)c3cc4cc5oc6ccccc6c5cc4c4ccccc34)c2)cc1]

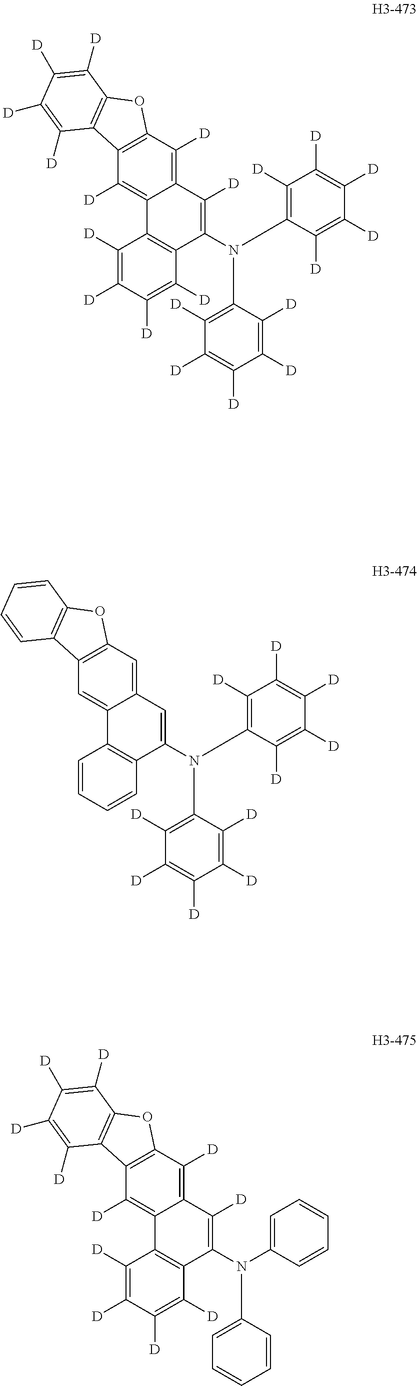 [SMILES: [2H]c1c([2H])c([2H])c(N(c2c([2H])c([2H])c([2H])c([2H])c2[2H])c2c([2H])c3c([2H])c4oc5c([2H])c([2H])c([2H])c([2H])c5c4c([2H])c3c3c([2H])c([2H])c([2H])c([2H])c23)c([2H])c1[2H] ; [2H]c1c([2H])c([2H])c(N(c2c([2H])c([2H])c([2H])c([2H])c2[2H])c2cc3cc4oc5ccccc5c4cc3c3ccccc23)c([2H])c1[2H] ; [2H]c1c([2H])c([2H])c2c(oc3c([2H])c4c([2H])c(N(c5ccccc5)c5ccccc5)c5c([2H])c([2H])c([2H])c([2H])c5c4c([2H])c32)c1[2H]]

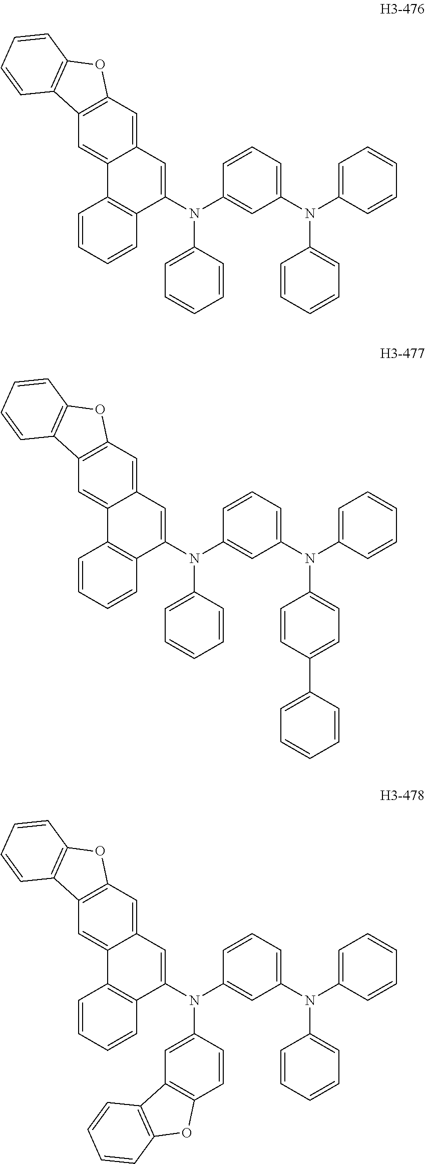 [SMILES: c1ccc(N(c2ccccc2)c2cccc(N(c3ccccc3)c3cc4cc5oc6ccccc6c5cc4c4ccccc34)c2)cc1 ; c1ccc(-c2ccc(N(c3ccccc3)c3cccc(N(c4ccccc4)c4cc5cc6oc7ccccc7c6cc5c5ccccc45)c3)cc2)cc1 ; c1ccc(N(c2ccccc2)c2cccc(N(c3ccc4oc5ccccc5c4c3)c3cc4cc5oc6ccccc6c5cc4c4ccccc34)c2)cc1]

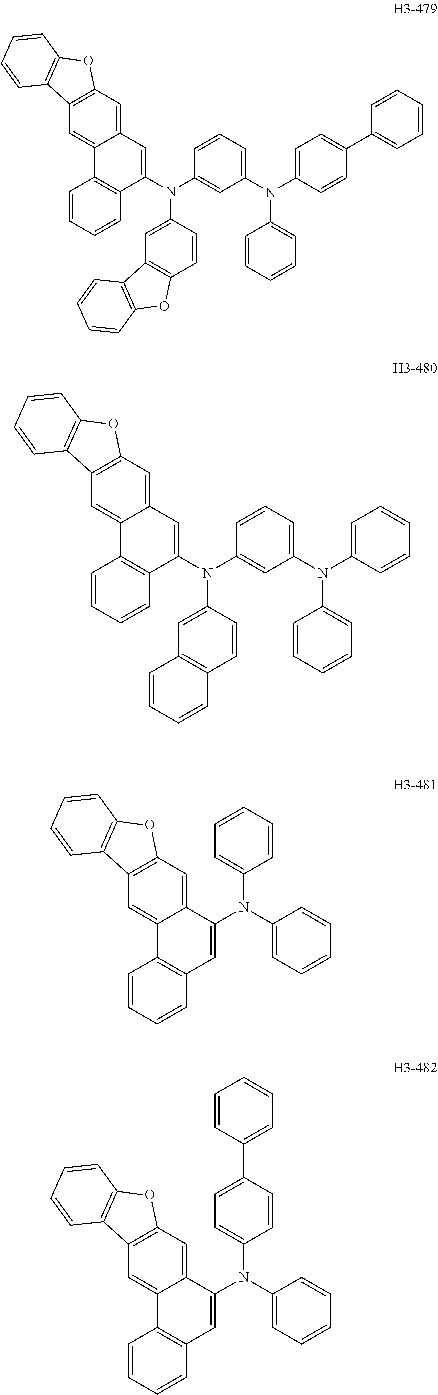 [SMILES: c1ccc(-c2ccc(N(c3ccccc3)c3cccc(N(c4ccc5oc6ccccc6c5c4)c4cc5cc6oc7ccccc7c6cc5c5ccccc45)c3)cc2)cc1 ; c1ccc(N(c2ccccc2)c2cccc(N(c3ccc4ccccc4c3)c3cc4cc5oc6ccccc6c5cc4c4ccccc34)c2)cc1 ; c1ccc(N(c2ccccc2)c2cc3ccccc3c3cc4c(cc23)oc2ccccc24)cc1 ; c1ccc(-c2ccc(N(c3ccccc3)c3cc4ccccc4c4cc5c(cc34)oc3ccccc35)cc2)cc1]

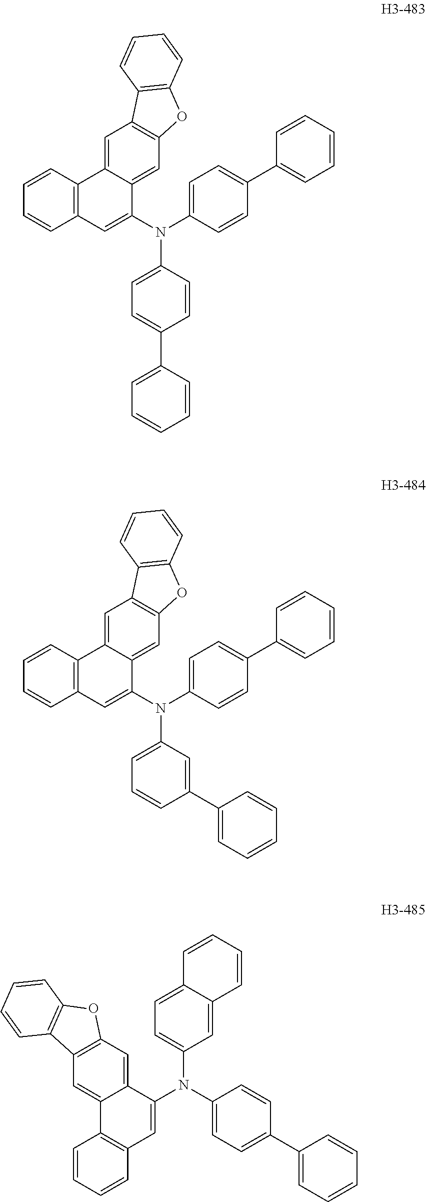 [SMILES: c1ccc(-c2ccc(N(c3ccc(-c4ccccc4)cc3)c3cc4ccccc4c4cc5c(cc34)oc3ccccc35)cc2)cc1 ; c1ccc(-c2ccc(N(c3cccc(-c4ccccc4)c3)c3cc4ccccc4c4cc5c(cc34)oc3ccccc35)cc2)cc1 ; c1ccc(-c2ccc(N(c3ccc4ccccc4c3)c3cc4ccccc4c4cc5c(cc34)oc3ccccc35)cc2)cc1]

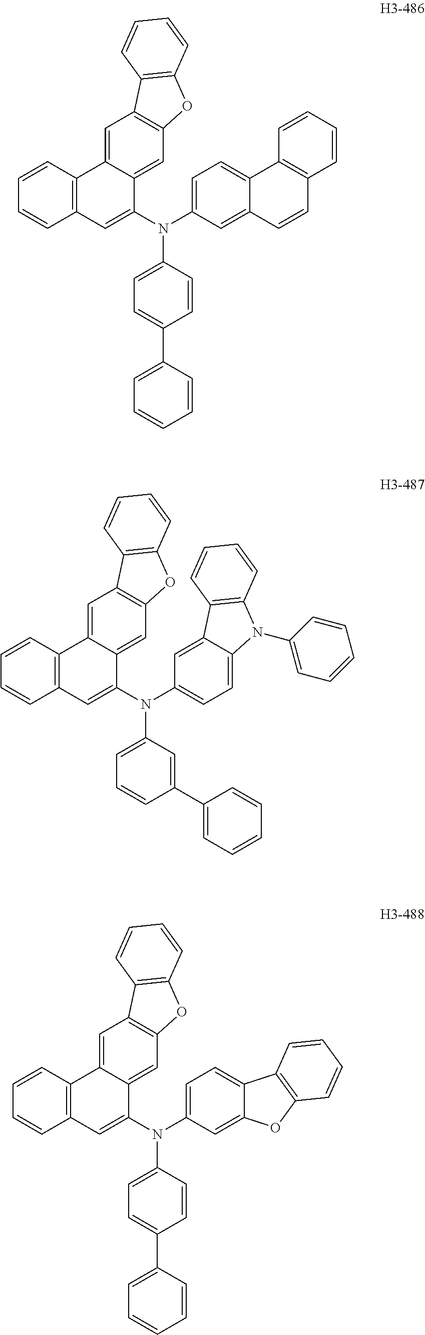 [SMILES: c1ccc(-c2ccc(N(c3ccc4c(ccc5ccccc54)c3)c3cc4ccccc4c4cc5c(cc34)oc3ccccc35)cc2)cc1 ; c1ccc(-c2cccc(N(c3ccc4c(c3)c3ccccc3n4-c3ccccc3)c3cc4ccccc4c4cc5c(cc34)oc3ccccc35)c2)cc1 ; c1ccc(-c2ccc(N(c3ccc4c(c3)oc3ccccc34)c3cc4ccccc4c4cc5c(cc34)oc3ccccc35)cc2)cc1]

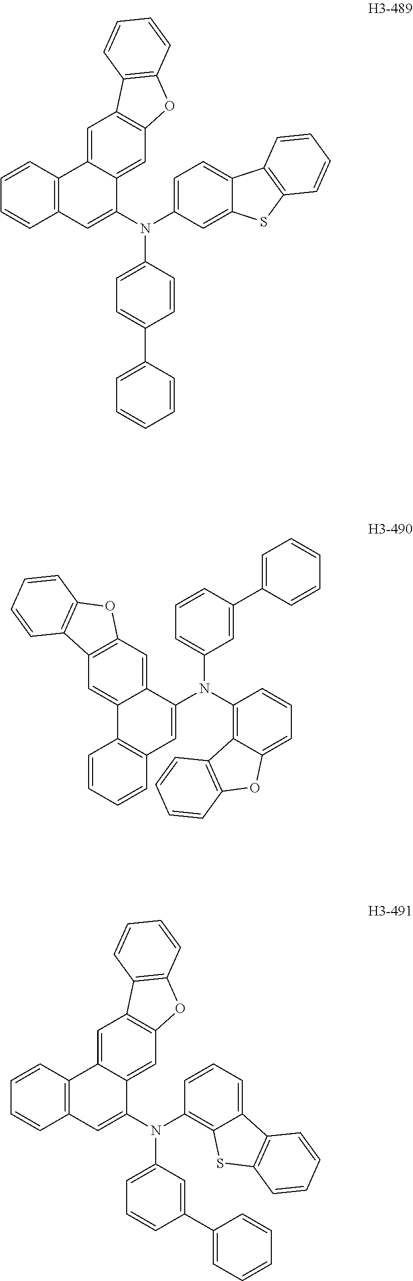 [SMILES: c1ccc(-c2ccc(N(c3ccc4c(c3)sc3ccccc34)c3cc4ccccc4c4cc5c(cc34)oc3ccccc35)cc2)cc1 ; c1ccc(-c2cccc(N(c3cc4ccccc4c4cc5c(cc34)oc3ccccc35)c3cccc4oc5ccccc5c34)c2)cc1 ; c1ccc(-c2cccc(N(c3cc4ccccc4c4cc5c(cc34)oc3ccccc35)c3cccc4c3sc3ccccc34)c2)cc1]

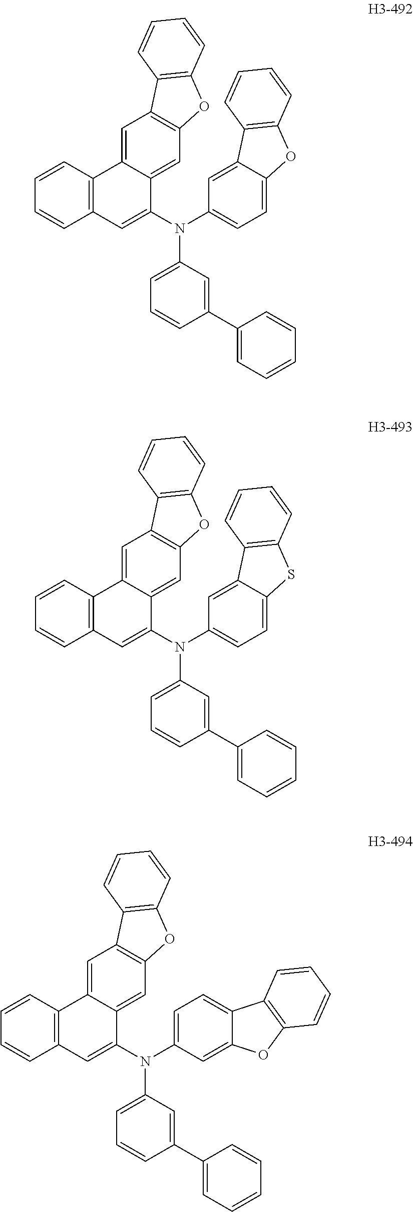 [SMILES: c1ccc(-c2cccc(N(c3ccc4oc5ccccc5c4c3)c3cc4ccccc4c4cc5c(cc34)oc3ccccc35)c2)cc1 ; c1ccc(-c2cccc(N(c3ccc4sc5ccccc5c4c3)c3cc4ccccc4c4cc5c(cc34)oc3ccccc35)c2)cc1 ; c1ccc(-c2cccc(N(c3ccc4c(c3)oc3ccccc34)c3cc4ccccc4c4cc5c(cc34)oc3ccccc35)c2)cc1]

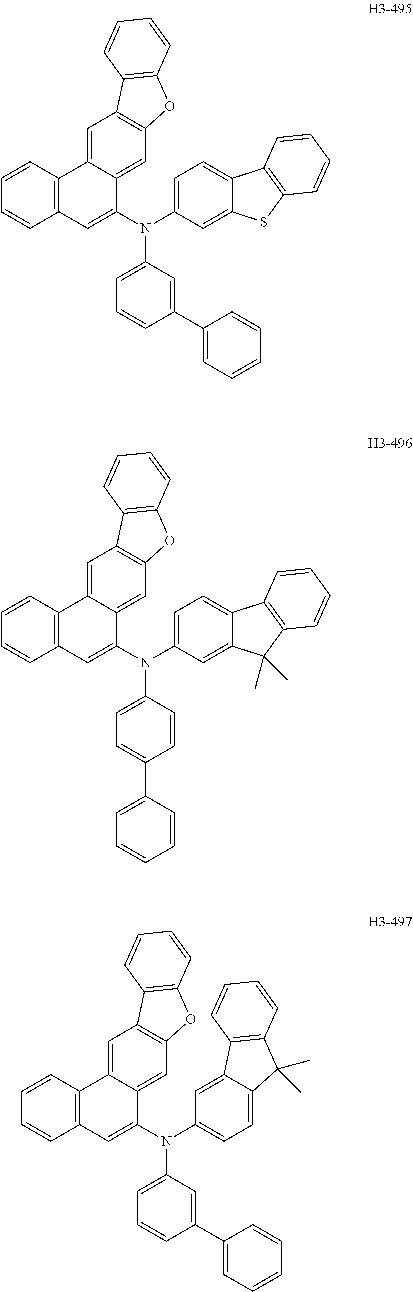 [SMILES: c1ccc(-c2cccc(N(c3ccc4c(c3)sc3ccccc34)c3cc4ccccc4c4cc5c(cc34)oc3ccccc35)c2)cc1 ; CC1(C)c2ccccc2-c2ccc(N(c3ccc(-c4ccccc4)cc3)c3cc4ccccc4c4cc5c(cc34)oc3ccccc35)cc21 ; CC1(C)c2ccccc2-c2cc(N(c3cccc(-c4ccccc4)c3)c3cc4ccccc4c4cc5c(cc34)oc3ccccc35)ccc21]

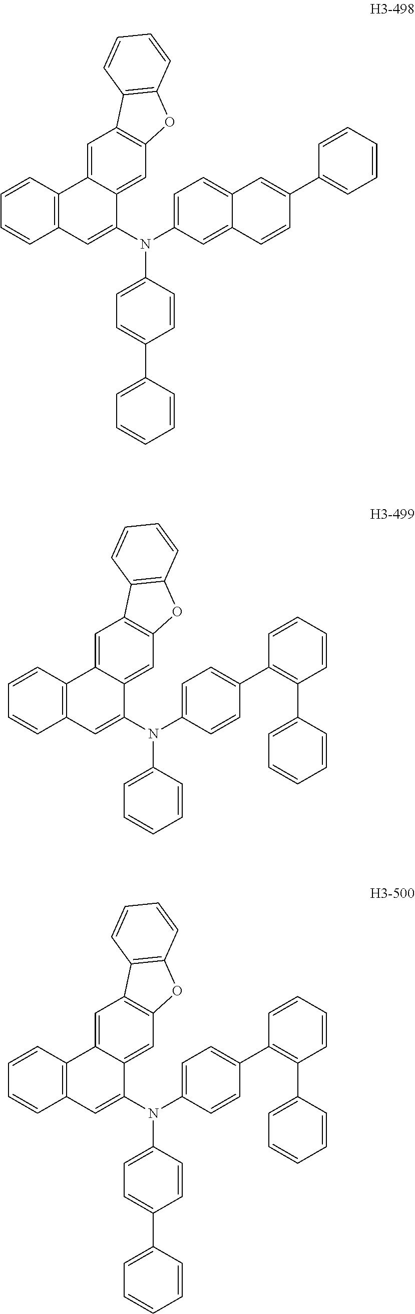 [SMILES: c1ccc(-c2ccc(N(c3ccc4cc(-c5ccccc5)ccc4c3)c3cc4ccccc4c4cc5c(cc34)oc3ccccc35)cc2)cc1 ; c1ccc(-c2ccccc2-c2ccc(N(c3ccccc3)c3cc4ccccc4c4cc5c(cc34)oc3ccccc35)cc2)cc1 ; c1ccc(-c2ccc(N(c3ccc(-c4ccccc4-c4ccccc4)cc3)c3cc4ccccc4c4cc5c(cc34)oc3ccccc35)cc2)cc1]

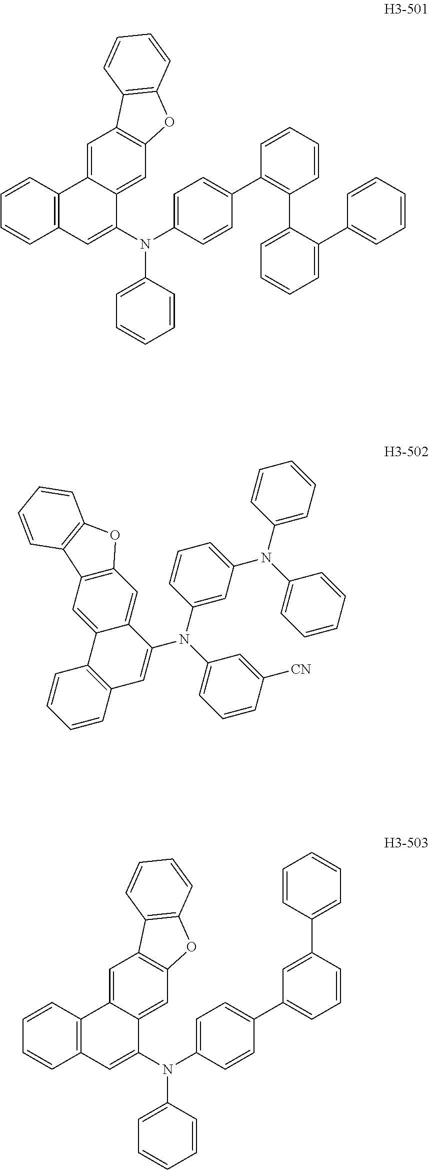 [SMILES: c1ccc(-c2ccccc2-c2ccccc2-c2ccc(N(c3ccccc3)c3cc4ccccc4c4cc5c(cc34)oc3ccccc35)cc2)cc1 ; N#Cc1cccc(N(c2cccc(N(c3ccccc3)c3ccccc3)c2)c2cc3ccccc3c3cc4c(cc23)oc2ccccc24)c1 ; c1ccc(-c2cccc(-c3ccc(N(c4ccccc4)c4cc5ccccc5c5cc6c(cc45)oc4ccccc46)cc3)c2)cc1]

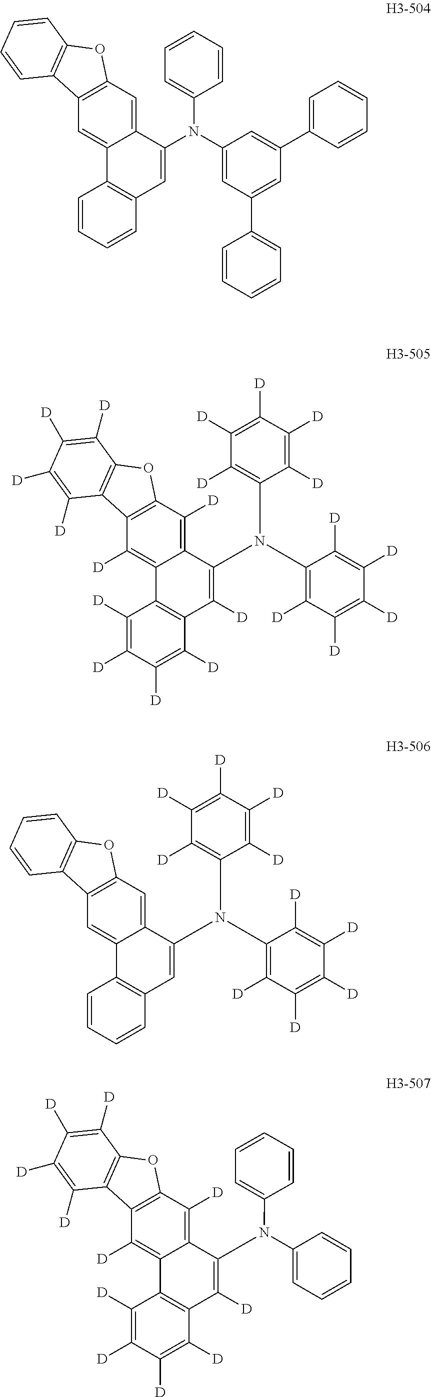 [SMILES: c1ccc(-c2cc(-c3ccccc3)cc(N(c3ccccc3)c3cc4ccccc4c4cc5c(cc34)oc3ccccc35)c2)cc1 ; [2H]c1c([2H])c([2H])c(N(c2c([2H])c([2H])c([2H])c([2H])c2[2H])c2c([2H])c3c([2H])c([2H])c([2H])c([2H])c3c3c([2H])c4c(oc5c([2H])c([2H])c([2H])c([2H])c54)c([2H])c23)c([2H])c1[2H] ; [2H]c1c([2H])c([2H])c(N(c2c([2H])c([2H])c([2H])c([2H])c2[2H])c2cc3ccccc3c3cc4c(cc23)oc2ccccc24)c([2H])c1[2H] ; [2H]c1c([2H])c([2H])c2c(oc3c([2H])c4c(N(c5ccccc5)c5ccccc5)c([2H])c5c([2H])c([2H])c([2H])c([2H])c5c4c([2H])c32)c1[2H]]

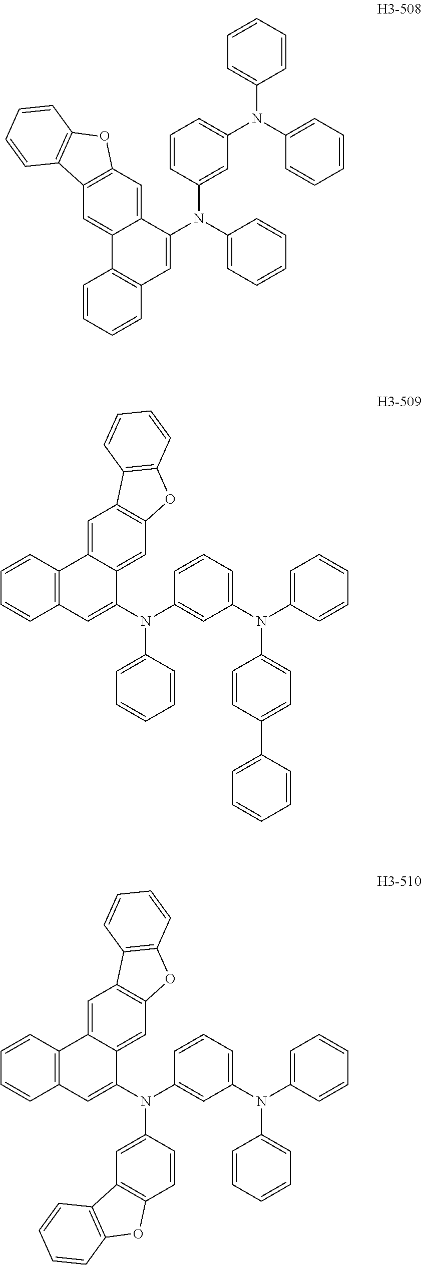 [SMILES: c1ccc(N(c2ccccc2)c2cccc(N(c3ccccc3)c3cc4ccccc4c4cc5c(cc34)oc3ccccc35)c2)cc1 ; c1ccc(-c2ccc(N(c3ccccc3)c3cccc(N(c4ccccc4)c4cc5ccccc5c5cc6c(cc45)oc4ccccc46)c3)cc2)cc1 ; c1ccc(N(c2ccccc2)c2cccc(N(c3ccc4oc5ccccc5c4c3)c3cc4ccccc4c4cc5c(cc34)oc3ccccc35)c2)cc1]

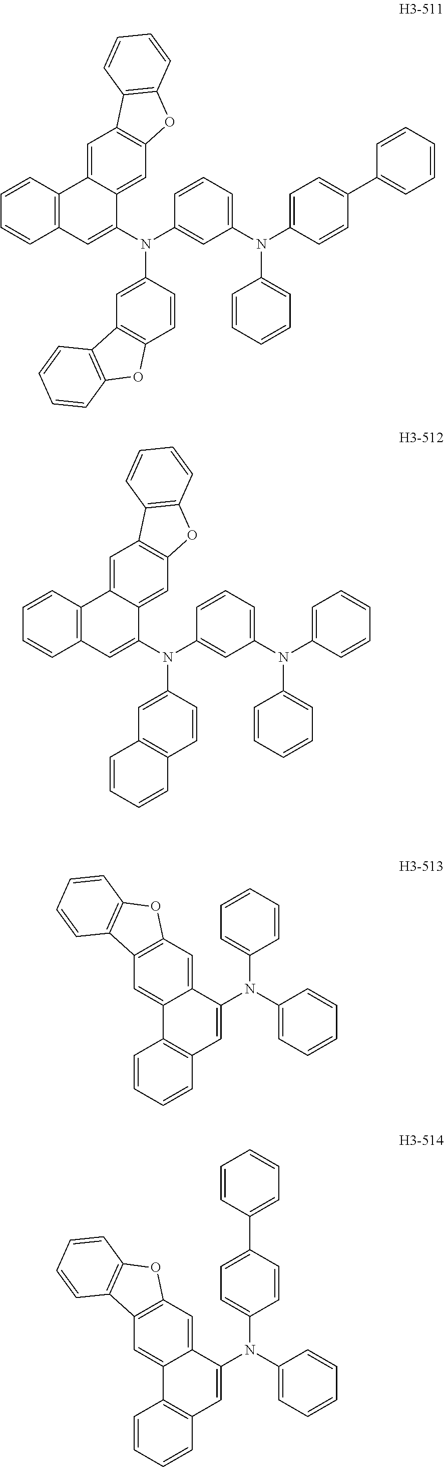 [SMILES: c1ccc(-c2ccc(N(c3ccccc3)c3cccc(N(c4ccc5oc6ccccc6c5c4)c4cc5ccccc5c5cc6c(cc45)oc4ccccc46)c3)cc2)cc1 ; c1ccc(N(c2ccccc2)c2cccc(N(c3ccc4ccccc4c3)c3cc4ccccc4c4cc5c(cc34)oc3ccccc35)c2)cc1 ; c1ccc(N(c2ccccc2)c2cc3ccccc3c3cc4c(cc23)oc2ccccc24)cc1 ; c1ccc(-c2ccc(N(c3ccccc3)c3cc4ccccc4c4cc5c(cc34)oc3ccccc35)cc2)cc1]

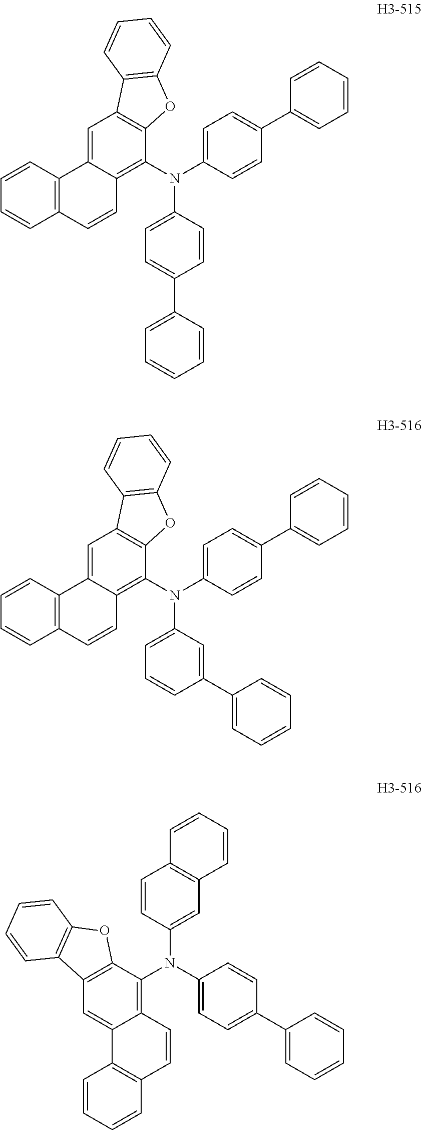 [SMILES: c1ccc(-c2ccc(N(c3ccc(-c4ccccc4)cc3)c3c4ccc5ccccc5c4cc4c3oc3ccccc34)cc2)cc1 ; c1ccc(-c2ccc(N(c3cccc(-c4ccccc4)c3)c3c4ccc5ccccc5c4cc4c3oc3ccccc34)cc2)cc1 ; c1ccc(-c2ccc(N(c3ccc4ccccc4c3)c3c4ccc5ccccc5c4cc4c3oc3ccccc34)cc2)cc1]

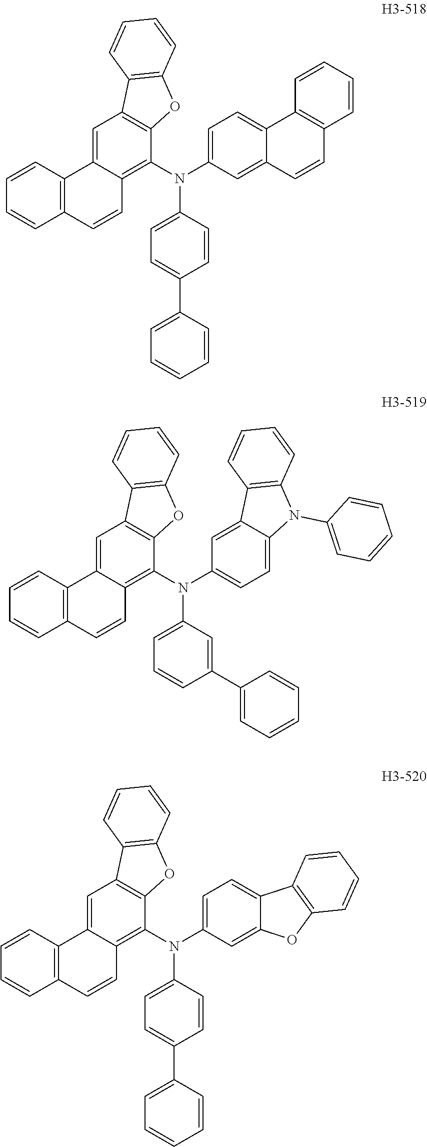 [SMILES: c1ccc(-c2ccc(N(c3ccc4c(ccc5ccccc54)c3)c3c4ccc5ccccc5c4cc4c3oc3ccccc34)cc2)cc1 ; c1ccc(-c2cccc(N(c3ccc4c(c3)c3ccccc3n4-c3ccccc3)c3c4ccc5ccccc5c4cc4c3oc3ccccc34)c2)cc1 ; c1ccc(-c2ccc(N(c3ccc4c(c3)oc3ccccc34)c3c4ccc5ccccc5c4cc4c3oc3ccccc34)cc2)cc1]

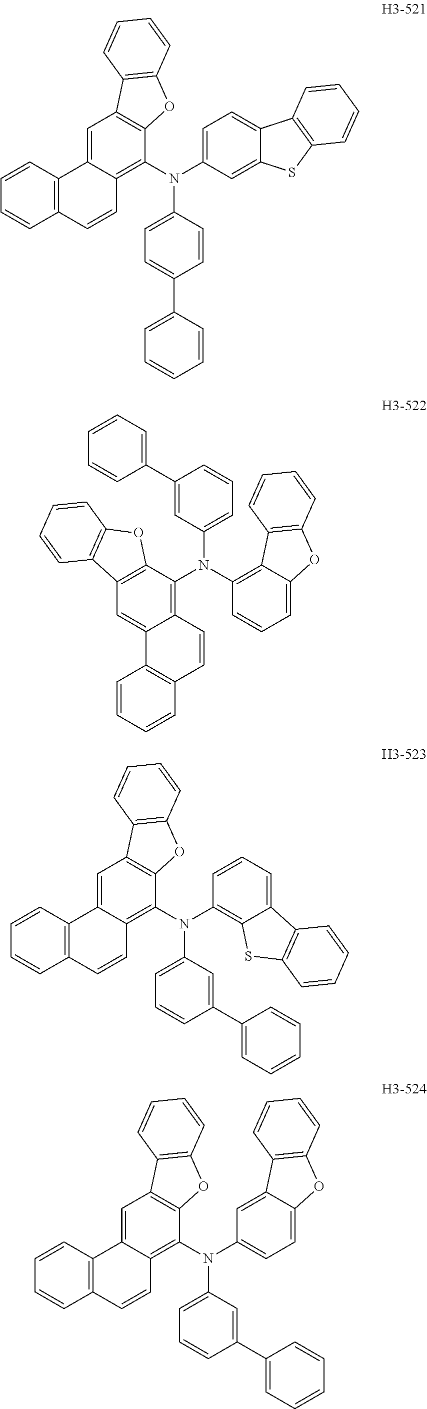

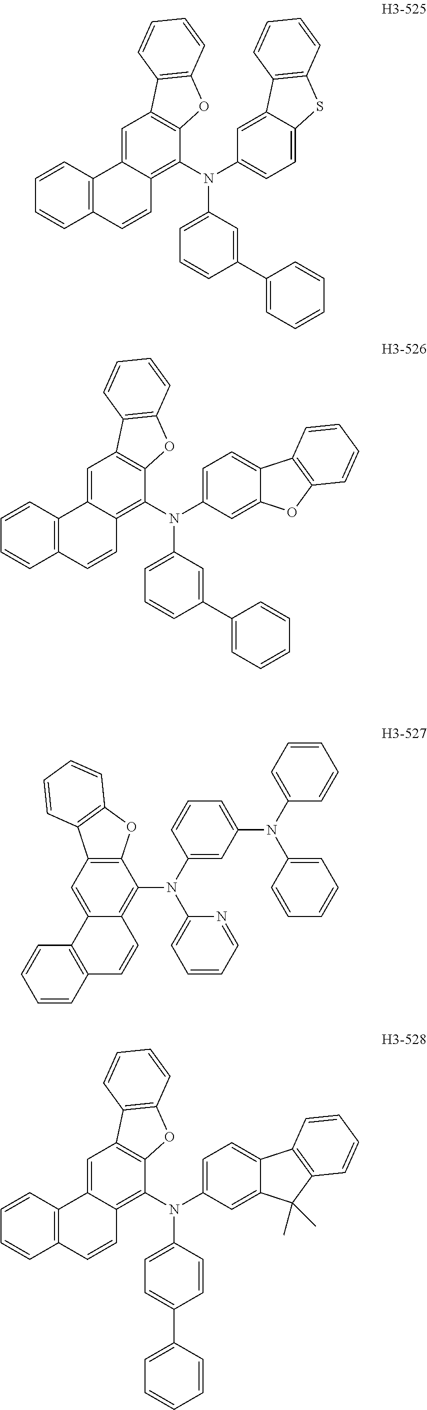 [SMILES: c1ccc(-c2cccc(N(c3ccc4sc5ccccc5c4c3)c3c4ccc5ccccc5c4cc4c3oc3ccccc34)c2)cc1 ; c1ccc(-c2cccc(N(c3ccc4c(c3)oc3ccccc34)c3c4ccc5ccccc5c4cc4c3oc3ccccc34)c2)cc1 ; c1ccc(N(c2ccccc2)c2cccc(N(c3ccccn3)c3c4ccc5ccccc5c4cc4c3oc3ccccc34)c2)cc1 ; CC1(C)c2ccccc2-c2ccc(N(c3ccc(-c4ccccc4)cc3)c3c4ccc5ccccc5c4cc4c3oc3ccccc34)cc21]

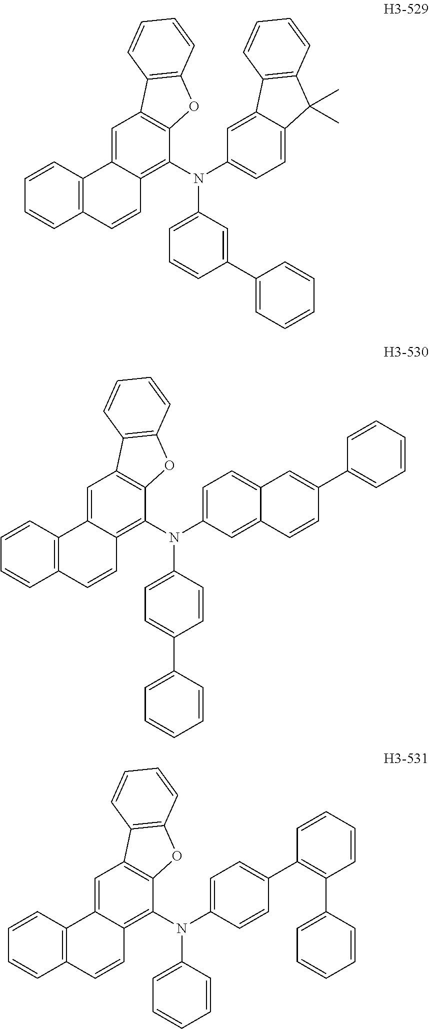

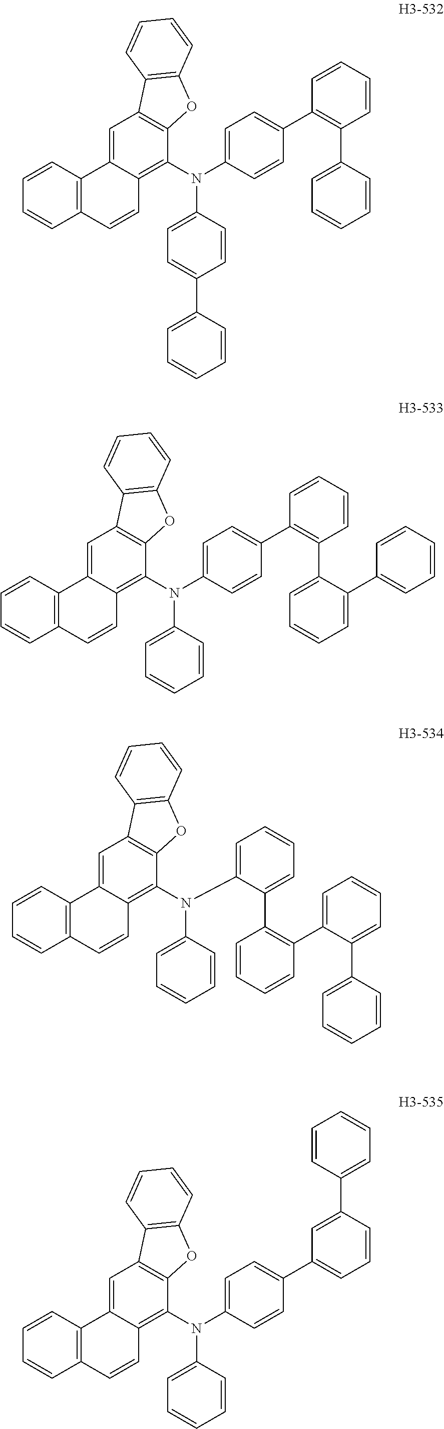 [SMILES: c1ccc(-c2ccc(N(c3ccc(-c4ccccc4-c4ccccc4)cc3)c3c4ccc5ccccc5c4cc4c3oc3ccccc34)cc2)cc1 ; c1ccc(-c2ccccc2-c2ccccc2-c2ccc(N(c3ccccc3)c3c4ccc5ccccc5c4cc4c3oc3ccccc34)cc2)cc1 ; c1ccc(-c2ccccc2-c2ccccc2-c2ccccc2N(c2ccccc2)c2c3ccc4ccccc4c3cc3c2oc2ccccc23)cc1 ; c1ccc(-c2cccc(-c3ccc(N(c4ccccc4)c4c5ccc6ccccc6c5cc5c4oc4ccccc45)cc3)c2)cc1]

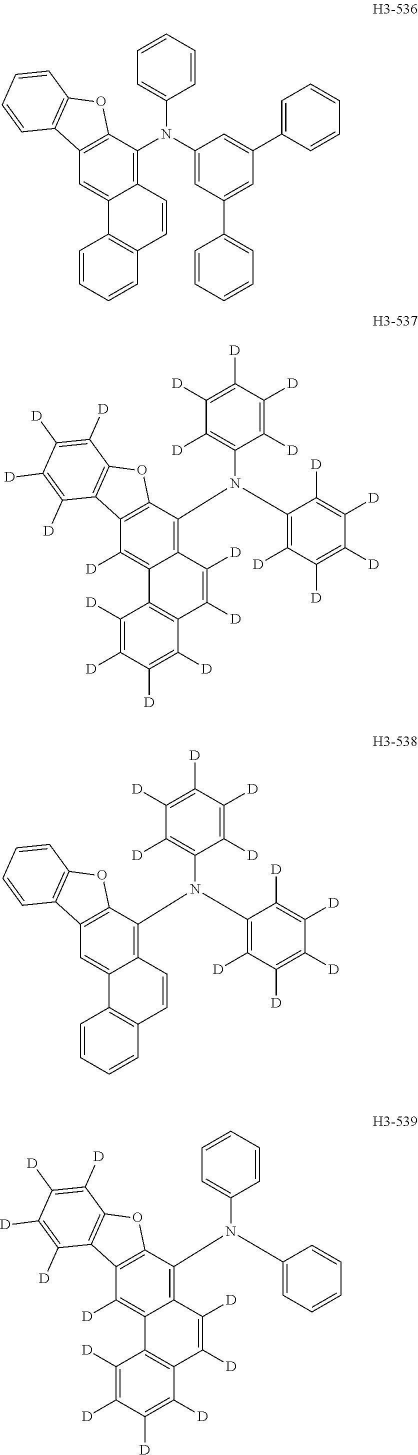 [SMILES: c1ccc(-c2cc(-c3ccccc3)cc(N(c3ccccc3)c3c4ccc5ccccc5c4cc4c3oc3ccccc34)c2)cc1 ; [2H]c1c([2H])c([2H])c(N(c2c([2H])c([2H])c([2H])c([2H])c2[2H])c2c3oc4c([2H])c([2H])c([2H])c([2H])c4c3c([2H])c3c2c([2H])c([2H])c2c([2H])c([2H])c([2H])c([2H])c23)c([2H])c1[2H] ; [2H]c1c([2H])c([2H])c(N(c2c([2H])c([2H])c([2H])c([2H])c2[2H])c2c3ccc4ccccc4c3cc3c2oc2ccccc23)c([2H])c1[2H] ; [2H]c1c([2H])c([2H])c2c(oc3c(N(c4ccccc4)c4ccccc4)c4c([2H])c([2H])c5c([2H])c([2H])c([2H])c([2H])c5c4c([2H])c32)c1[2H]]

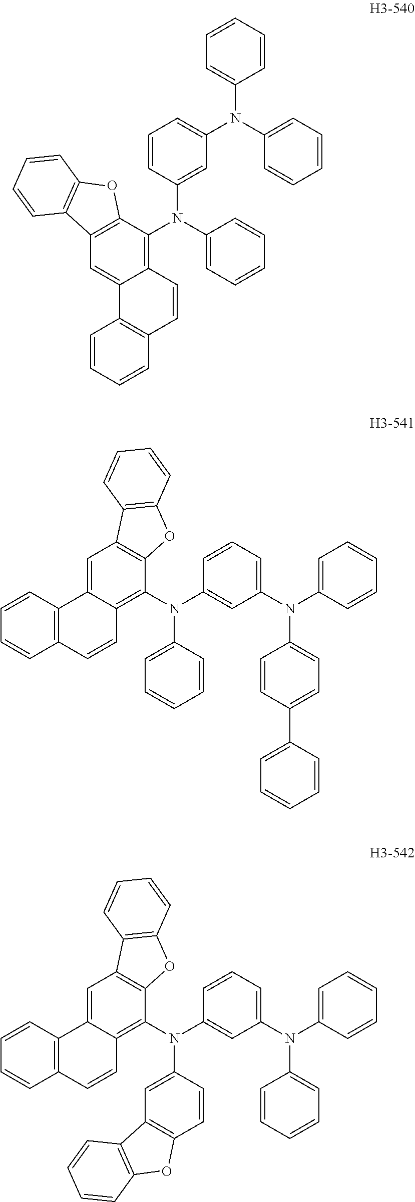 [SMILES: c1ccc(N(c2ccccc2)c2cccc(N(c3ccccc3)c3c4ccc5ccccc5c4cc4c3oc3ccccc34)c2)cc1 ; c1ccc(-c2ccc(N(c3ccccc3)c3cccc(N(c4ccccc4)c4c5ccc6ccccc6c5cc5c4oc4ccccc45)c3)cc2)cc1 ; c1ccc(N(c2ccccc2)c2cccc(N(c3ccc4oc5ccccc5c4c3)c3c4ccc5ccccc5c4cc4c3oc3ccccc34)c2)cc1]

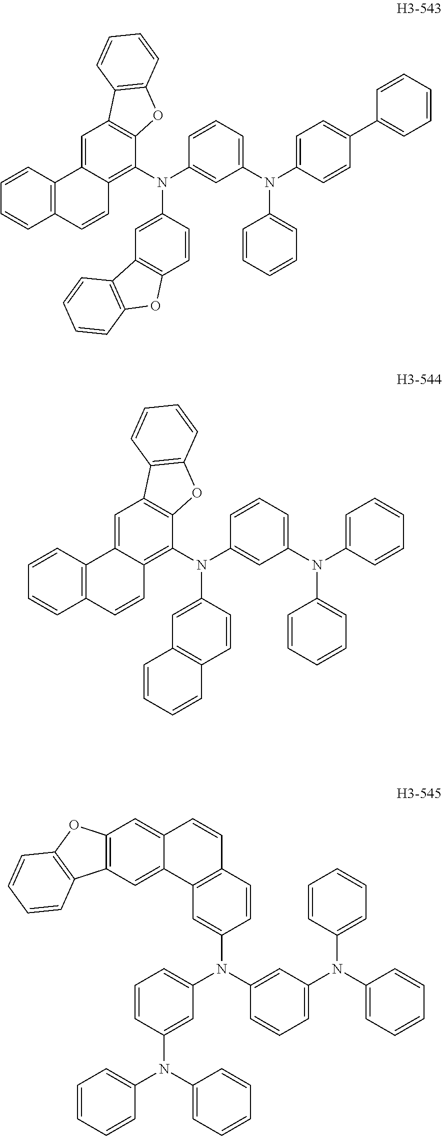 [SMILES: c1ccc(-c2ccc(N(c3ccccc3)c3cccc(N(c4ccc5oc6ccccc6c5c4)c4c5ccc6ccccc6c5cc5c4oc4ccccc45)c3)cc2)cc1 ; c1ccc(N(c2ccccc2)c2cccc(N(c3ccc4ccccc4c3)c3c4ccc5ccccc5c4cc4c3oc3ccccc34)c2)cc1 ; c1ccc(N(c2ccccc2)c2cccc(N(c3cccc(N(c4ccccc4)c4ccccc4)c3)c3ccc4ccc5cc6oc7ccccc7c6cc5c4c3)c2)cc1]

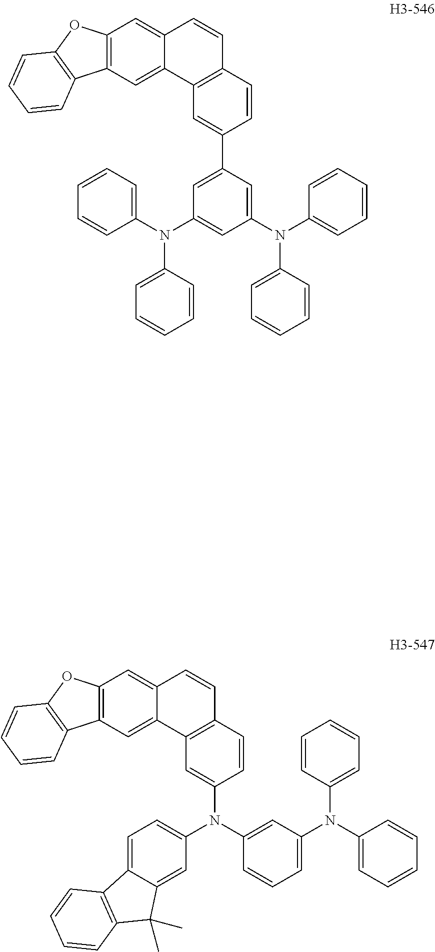 [SMILES: c1ccc(N(c2ccccc2)c2cc(-c3ccc4ccc5cc6oc7ccccc7c6cc5c4c3)cc(N(c3ccccc3)c3ccccc3)c2)cc1 ; CC1(C)c2ccccc2-c2ccc(N(c3cccc(N(c4ccccc4)c4ccccc4)c3)c3ccc4ccc5cc6oc7ccccc7c6cc5c4c3)cc21]

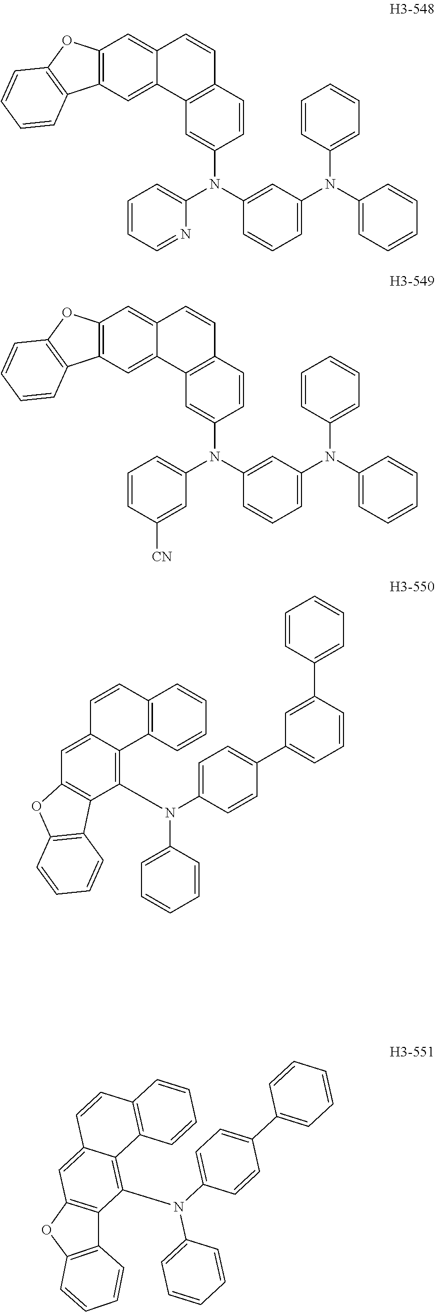 [SMILES: c1ccc(N(c2ccccc2)c2cccc(N(c3ccc4ccc5cc6oc7ccccc7c6cc5c4c3)c3ccccn3)c2)cc1 ; N#Cc1cccc(N(c2cccc(N(c3ccccc3)c3ccccc3)c2)c2ccc3ccc4cc5oc6ccccc6c5cc4c3c2)c1 ; c1ccc(-c2cccc(-c3ccc(N(c4ccccc4)c4c5c(ccc6ccccc65)cc5oc6ccccc6c45)cc3)c2)cc1 ; c1ccc(-c2ccc(N(c3ccccc3)c3c4c(ccc5ccccc54)cc4oc5ccccc5c34)cc2)cc1]

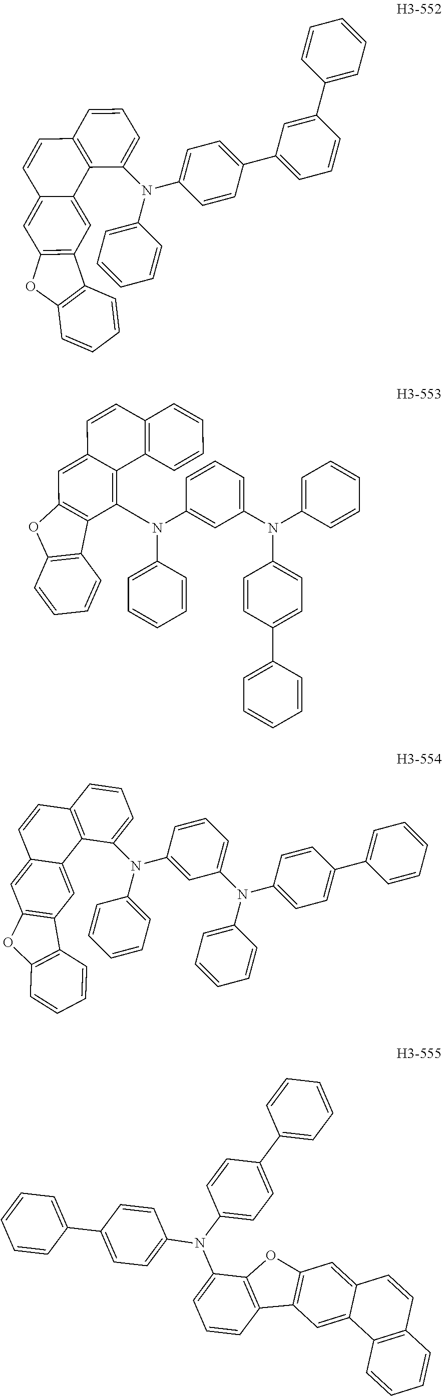 [SMILES: c1ccc(-c2cccc(-c3ccc(N(c4ccccc4)c4cccc5ccc6cc7oc8ccccc8c7cc6c45)cc3)c2)cc1 ; c1ccc(-c2ccc(N(c3ccccc3)c3cccc(N(c4ccccc4)c4c5c(ccc6ccccc65)cc5oc6ccccc6c45)c3)cc2)cc1 ; c1ccc(-c2ccc(N(c3ccccc3)c3cccc(N(c4ccccc4)c4cccc5ccc6cc7oc8ccccc8c7cc6c45)c3)cc2)cc1 ; c1ccc(-c2ccc(N(c3ccc(-c4ccccc4)cc3)c3cccc4c3oc3cc5ccc6ccccc6c5cc34)cc2)cc1]

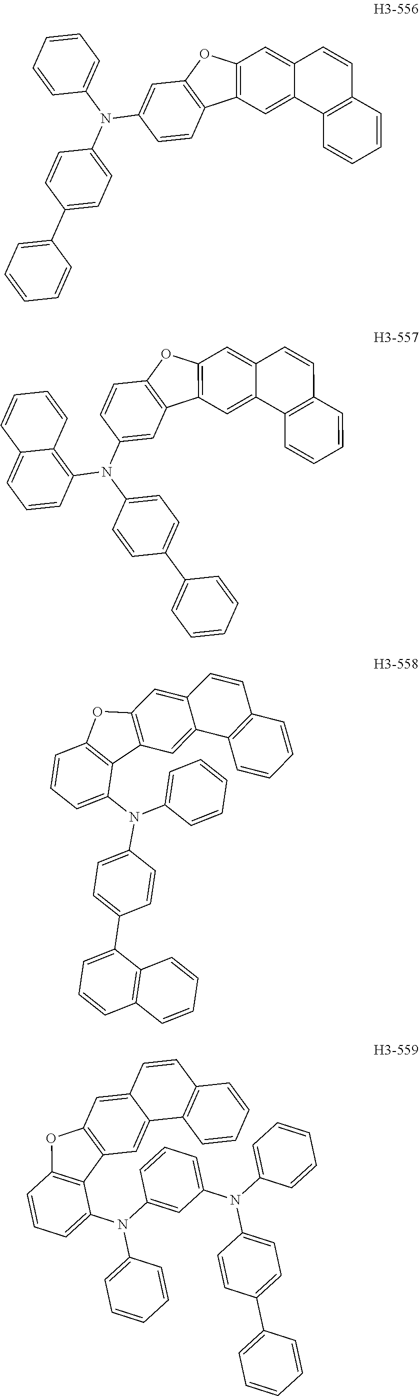

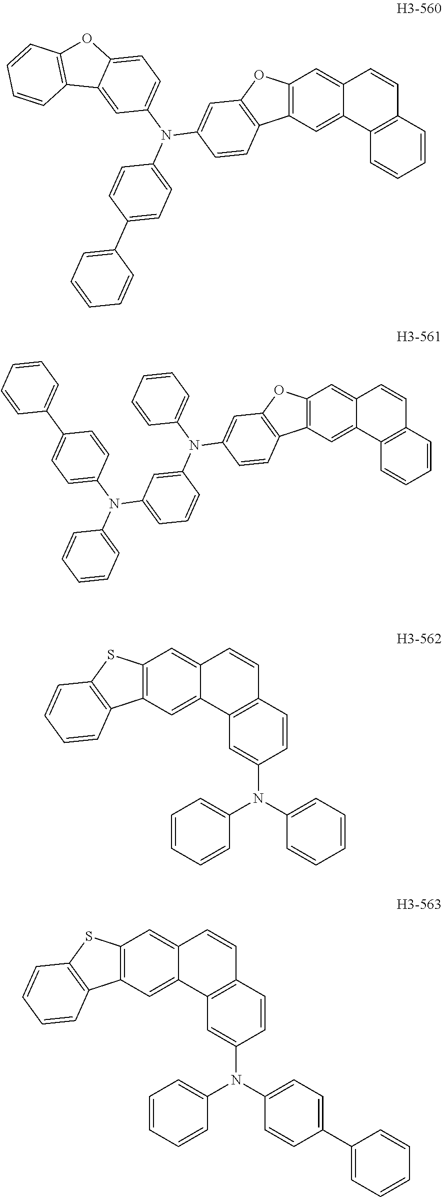 [SMILES: c1ccc(-c2ccc(N(c3ccc4c(c3)oc3cc5ccc6ccccc6c5cc34)c3ccc4oc5ccccc5c4c3)cc2)cc1 ; c1ccc(-c2ccc(N(c3ccccc3)c3cccc(N(c4ccccc4)c4ccc5c(c4)oc4cc6ccc7ccccc7c6cc45)c3)cc2)cc1 ; c1ccc(N(c2ccccc2)c2ccc3ccc4cc5sc6ccccc6c5cc4c3c2)cc1 ; c1ccc(-c2ccc(N(c3ccccc3)c3ccc4ccc5cc6sc7ccccc7c6cc5c4c3)cc2)cc1]

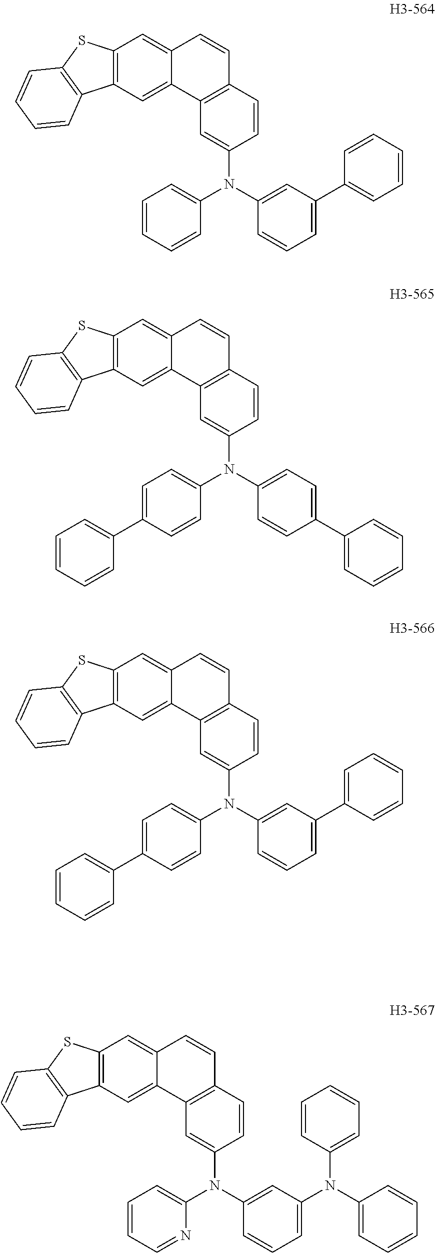 [SMILES: c1ccc(-c2cccc(N(c3ccccc3)c3ccc4ccc5cc6sc7ccccc7c6cc5c4c3)c2)cc1 ; c1ccc(-c2ccc(N(c3ccc(-c4ccccc4)cc3)c3ccc4ccc5cc6sc7ccccc7c6cc5c4c3)cc2)cc1 ; c1ccc(-c2ccc(N(c3cccc(-c4ccccc4)c3)c3ccc4ccc5cc6sc7ccccc7c6cc5c4c3)cc2)cc1 ; c1ccc(N(c2ccccc2)c2cccc(N(c3ccc4ccc5cc6sc7ccccc7c6cc5c4c3)c3ccccn3)c2)cc1]

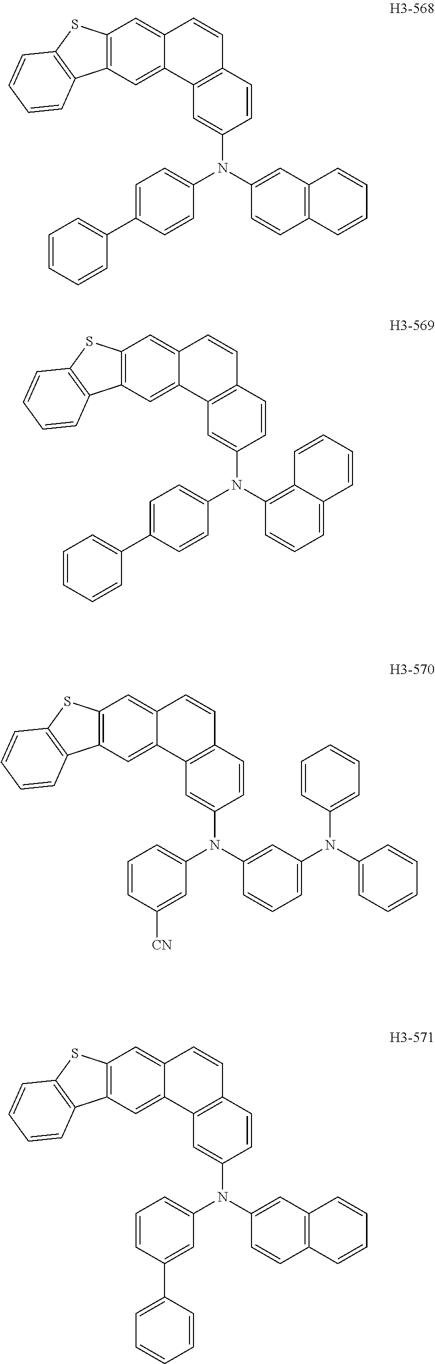 [SMILES: c1ccc(-c2ccc(N(c3ccc4ccccc4c3)c3ccc4ccc5cc6sc7ccccc7c6cc5c4c3)cc2)cc1 ; c1ccc(-c2ccc(N(c3ccc4ccc5cc6sc7ccccc7c6cc5c4c3)c3cccc4ccccc34)cc2)cc1 ; N#Cc1cccc(N(c2cccc(N(c3ccccc3)c3ccccc3)c2)c2ccc3ccc4cc5sc6ccccc6c5cc4c3c2)c1 ; c1ccc(-c2cccc(N(c3ccc4ccccc4c3)c3ccc4ccc5cc6sc7ccccc7c6cc5c4c3)c2)cc1]

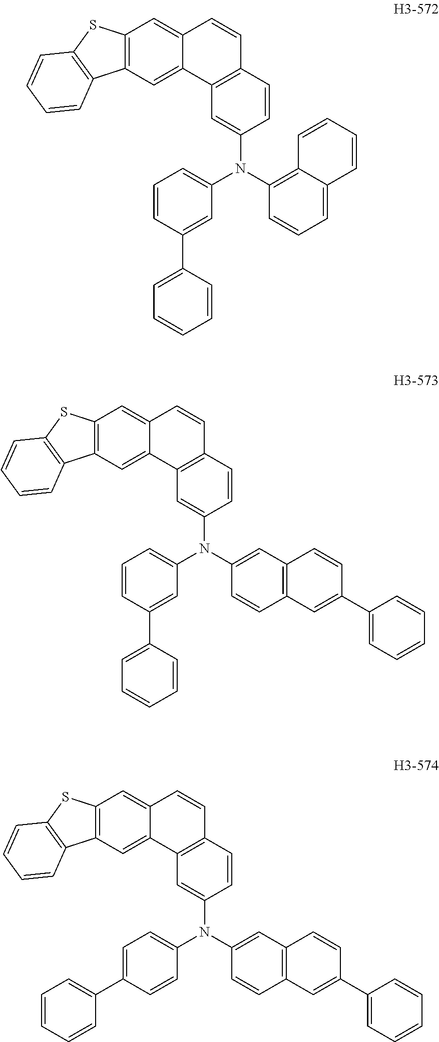 [SMILES: c1ccc(-c2cccc(N(c3ccc4ccc5cc6sc7ccccc7c6cc5c4c3)c3cccc4ccccc34)c2)cc1 ; c1ccc(-c2cccc(N(c3ccc4cc(-c5ccccc5)ccc4c3)c3ccc4ccc5cc6sc7ccccc7c6cc5c4c3)c2)cc1 ; c1ccc(-c2ccc(N(c3ccc4cc(-c5ccccc5)ccc4c3)c3ccc4ccc5cc6sc7ccccc7c6cc5c4c3)cc2)cc1]

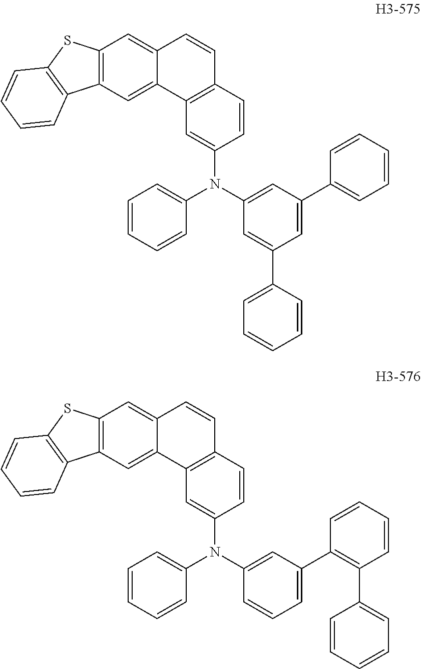 [SMILES: c1ccc(-c2cc(-c3ccccc3)cc(N(c3ccccc3)c3ccc4ccc5cc6sc7ccccc7c6cc5c4c3)c2)cc1 ; c1ccc(-c2ccccc2-c2cccc(N(c3ccccc3)c3ccc4ccc5cc6sc7ccccc7c6cc5c4c3)c2)cc1]

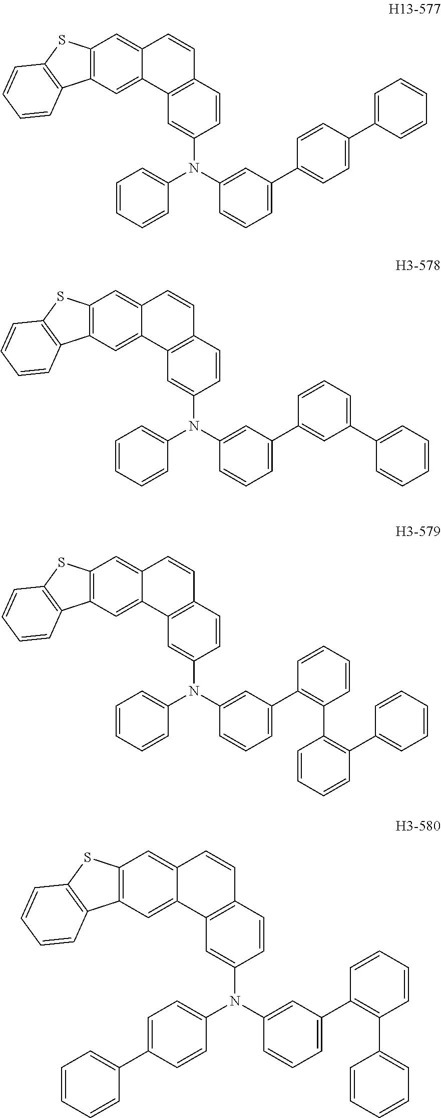 [SMILES: c1ccc(-c2ccc(-c3cccc(N(c4ccccc4)c4ccc5ccc6cc7sc8ccccc8c7cc6c5c4)c3)cc2)cc1 ; c1ccc(-c2cccc(-c3cccc(N(c4ccccc4)c4ccc5ccc6cc7sc8ccccc8c7cc6c5c4)c3)c2)cc1 ; c1ccc(-c2ccccc2-c2ccccc2-c2cccc(N(c3ccccc3)c3ccc4ccc5cc6sc7ccccc7c6cc5c4c3)c2)cc1 ; c1ccc(-c2ccc(N(c3cccc(-c4ccccc4-c4ccccc4)c3)c3ccc4ccc5cc6sc7ccccc7c6cc5c4c3)cc2)cc1]

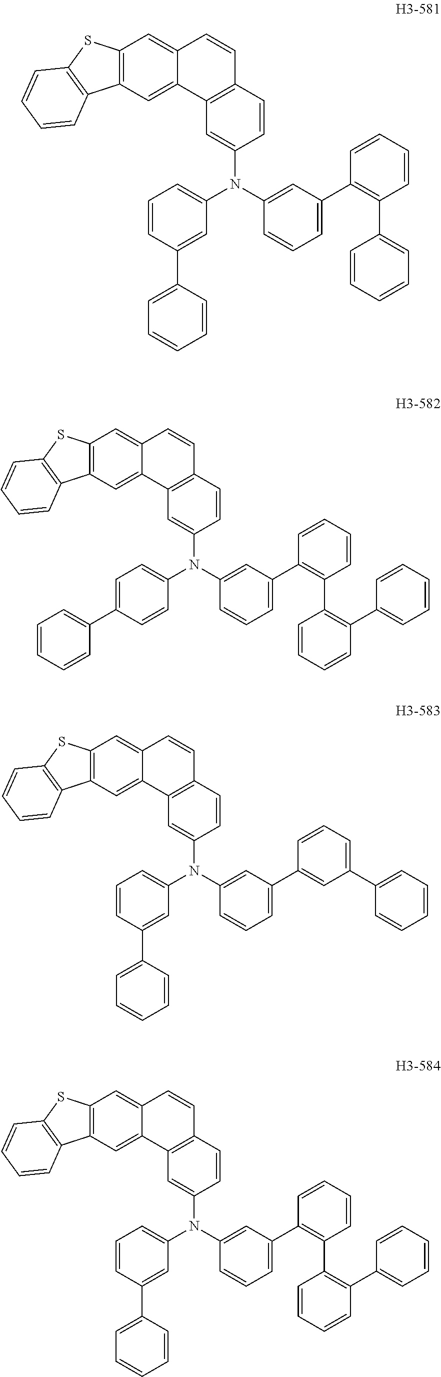 [SMILES: c1ccc(-c2cccc(N(c3cccc(-c4ccccc4-c4ccccc4)c3)c3ccc4ccc5cc6sc7ccccc7c6cc5c4c3)c2)cc1 ; c1ccc(-c2ccc(N(c3cccc(-c4ccccc4-c4ccccc4-c4ccccc4)c3)c3ccc4ccc5cc6sc7ccccc7c6cc5c4c3)cc2)cc1 ; c1ccc(-c2cccc(-c3cccc(N(c4cccc(-c5ccccc5)c4)c4ccc5ccc6cc7sc8ccccc8c7cc6c5c4)c3)c2)cc1 ; c1ccc(-c2cccc(N(c3cccc(-c4ccccc4-c4ccccc4-c4ccccc4)c3)c3ccc4ccc5cc6sc7ccccc7c6cc5c4c3)c2)cc1]

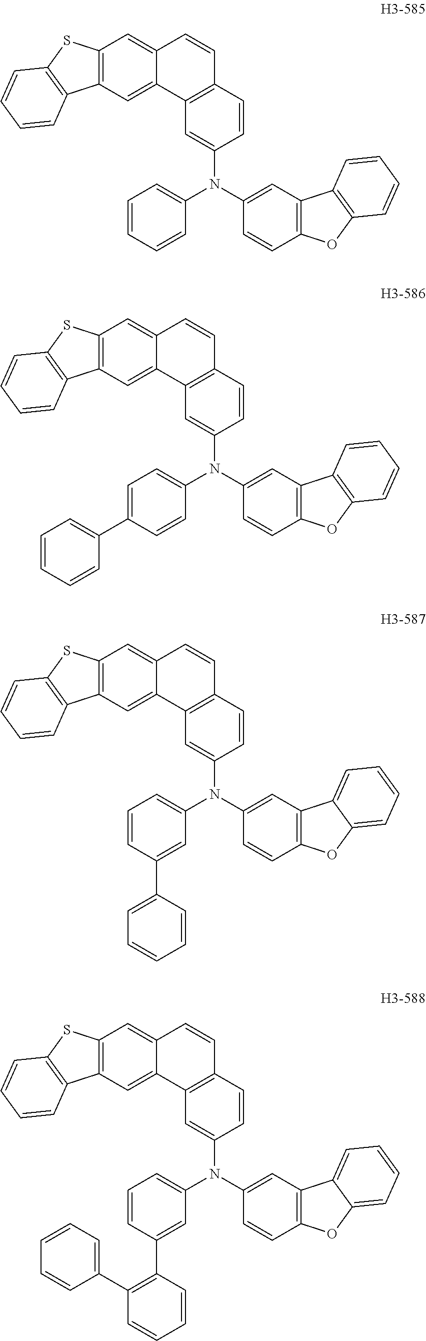 [SMILES: c1ccc(N(c2ccc3ccc4cc5sc6ccccc6c5cc4c3c2)c2ccc3oc4ccccc4c3c2)cc1 ; c1ccc(-c2ccc(N(c3ccc4ccc5cc6sc7ccccc7c6cc5c4c3)c3ccc4oc5ccccc5c4c3)cc2)cc1 ; c1ccc(-c2cccc(N(c3ccc4ccc5cc6sc7ccccc7c6cc5c4c3)c3ccc4oc5ccccc5c4c3)c2)cc1 ; c1ccc(-c2ccccc2-c2cccc(N(c3ccc4ccc5cc6sc7ccccc7c6cc5c4c3)c3ccc4oc5ccccc5c4c3)c2)cc1]

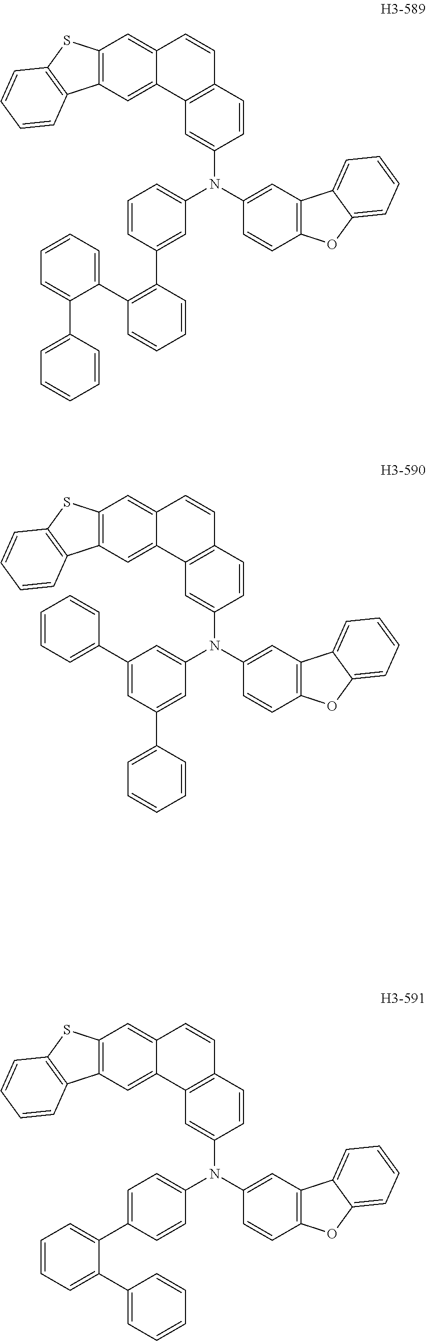 [SMILES: c1ccc(-c2ccccc2-c2ccccc2-c2cccc(N(c3ccc4ccc5cc6sc7ccccc7c6cc5c4c3)c3ccc4oc5ccccc5c4c3)c2)cc1 ; c1ccc(-c2cc(-c3ccccc3)cc(N(c3ccc4ccc5cc6sc7ccccc7c6cc5c4c3)c3ccc4oc5ccccc5c4c3)c2)cc1 ; c1ccc(-c2ccccc2-c2ccc(N(c3ccc4ccc5cc6sc7ccccc7c6cc5c4c3)c3ccc4oc5ccccc5c4c3)cc2)cc1]

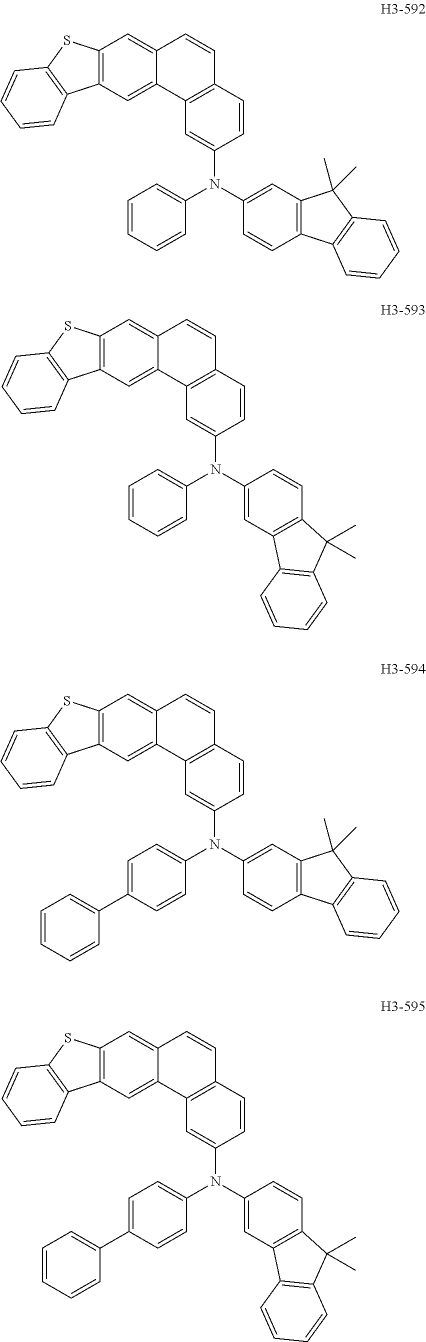 [SMILES: CC1(C)c2ccccc2-c2ccc(N(c3ccccc3)c3ccc4ccc5cc6sc7ccccc7c6cc5c4c3)cc21 ; CC1(C)c2ccccc2-c2cc(N(c3ccccc3)c3ccc4ccc5cc6sc7ccccc7c6cc5c4c3)ccc21 ; CC1(C)c2ccccc2-c2ccc(N(c3ccc(-c4ccccc4)cc3)c3ccc4ccc5cc6sc7ccccc7c6cc5c4c3)cc21 ; CC1(C)c2ccccc2-c2cc(N(c3ccc(-c4ccccc4)cc3)c3ccc4ccc5cc6sc7ccccc7c6cc5c4c3)ccc21]

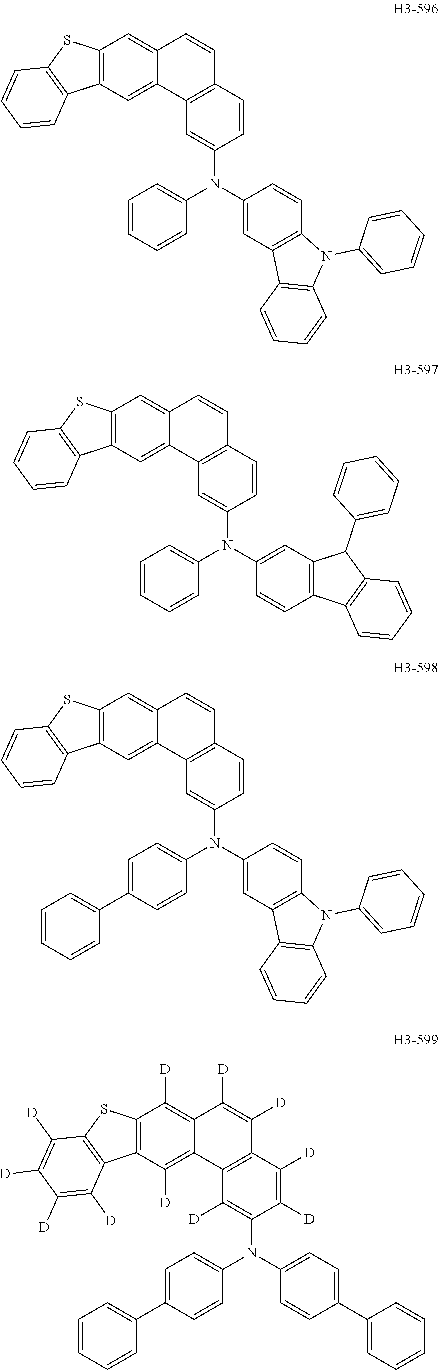 [SMILES: c1ccc(N(c2ccc3ccc4cc5sc6ccccc6c5cc4c3c2)c2ccc3c(c2)c2ccccc2n3-c2ccccc2)cc1 ; c1ccc(C2c3ccccc3-c3ccc(N(c4ccccc4)c4ccc5ccc6cc7sc8ccccc8c7cc6c5c4)cc32)cc1 ; c1ccc(-c2ccc(N(c3ccc4ccc5cc6sc7ccccc7c6cc5c4c3)c3ccc4c(c3)c3ccccc3n4-c3ccccc3)cc2)cc1 ; [2H]c1c([2H])c([2H])c2c(sc3c([2H])c4c([2H])c([2H])c5c([2H])c([2H])c(N(c6ccc(-c7ccccc7)cc6)c6ccc(-c7ccccc7)cc6)c([2H])c5c4c([2H])c32)c1[2H]]

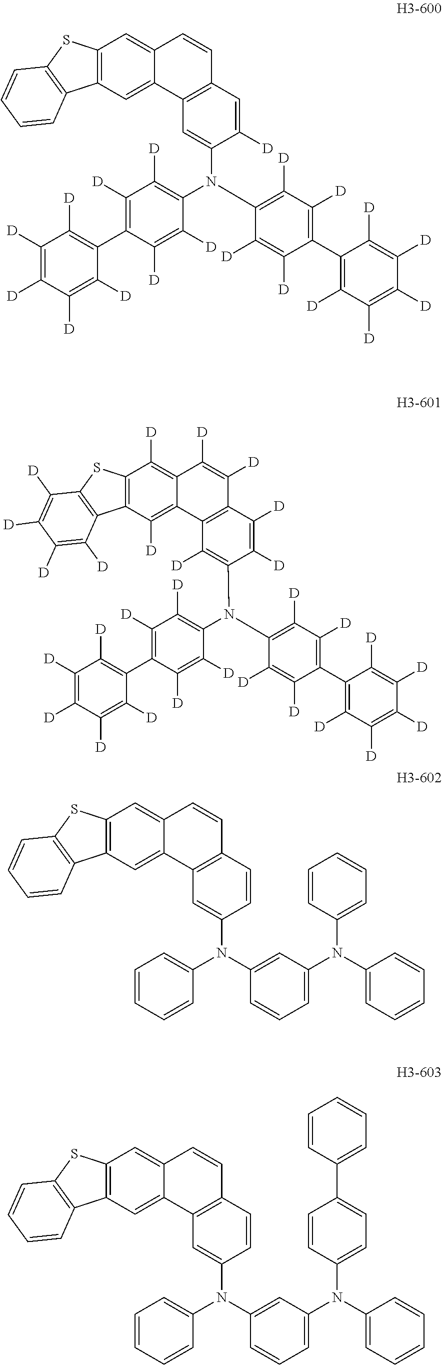

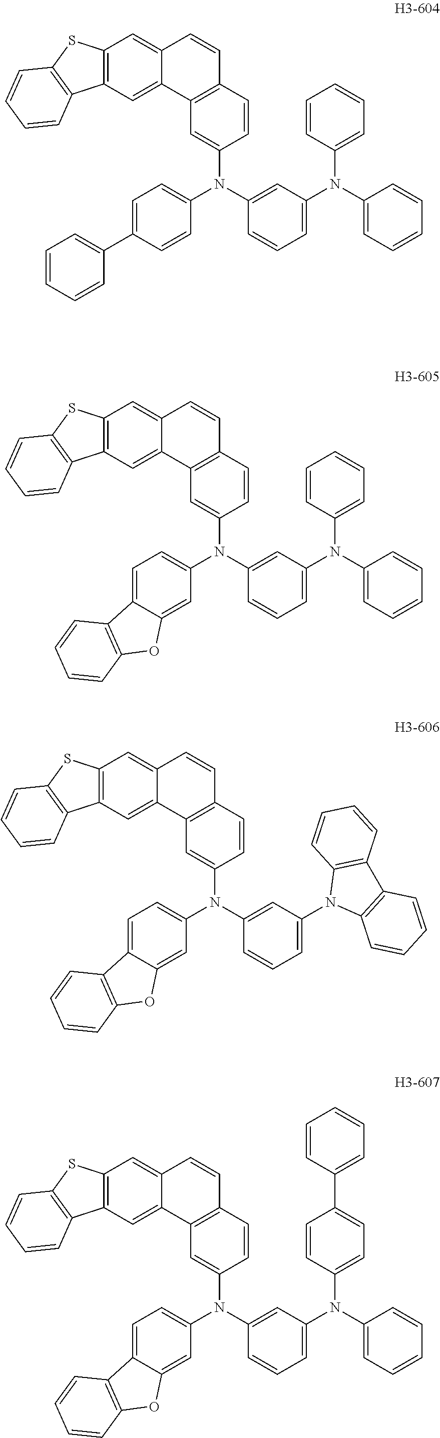 [SMILES: c1ccc(-c2ccc(N(c3cccc(N(c4ccccc4)c4ccccc4)c3)c3ccc4ccc5cc6sc7ccccc7c6cc5c4c3)cc2)cc1 ; c1ccc(N(c2ccccc2)c2cccc(N(c3ccc4c(c3)oc3ccccc34)c3ccc4ccc5cc6sc7ccccc7c6cc5c4c3)c2)cc1 ; c1cc(N(c2ccc3c(c2)oc2ccccc23)c2ccc3ccc4cc5sc6ccccc6c5cc4c3c2)cc(-n2c3ccccc3c3ccccc32)c1 ; c1ccc(-c2ccc(N(c3ccccc3)c3cccc(N(c4ccc5c(c4)oc4ccccc45)c4ccc5ccc6cc7sc8ccccc8c7cc6c5c4)c3)cc2)cc1]

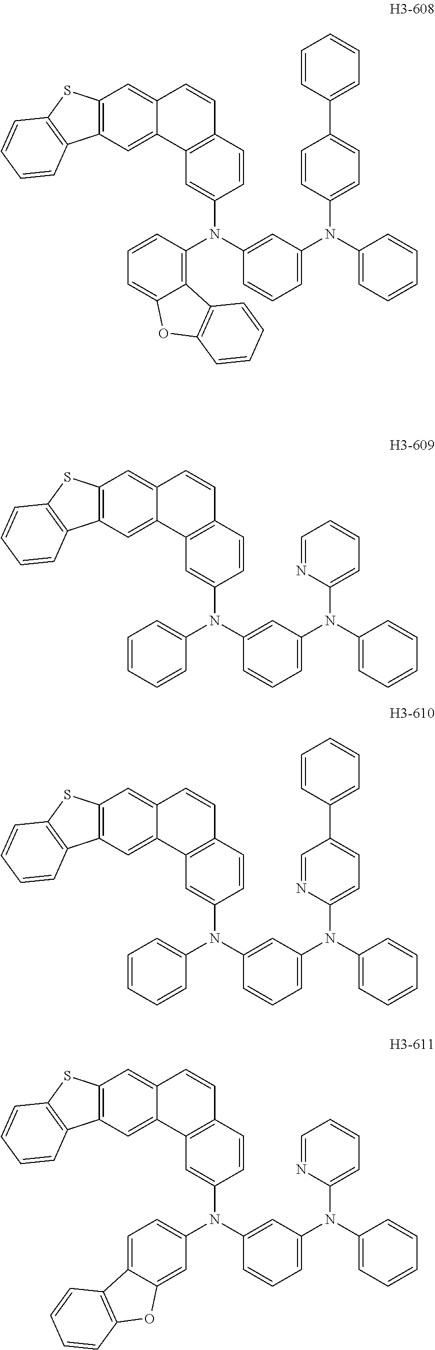 [SMILES: c1ccc(-c2ccc(N(c3ccccc3)c3cccc(N(c4ccc5ccc6cc7sc8ccccc8c7cc6c5c4)c4cccc5oc6ccccc6c45)c3)cc2)cc1 ; c1ccc(N(c2cccc(N(c3ccccc3)c3ccccn3)c2)c2ccc3ccc4cc5sc6ccccc6c5cc4c3c2)cc1 ; c1ccc(-c2ccc(N(c3ccccc3)c3cccc(N(c4ccccc4)c4ccc5ccc6cc7sc8ccccc8c7cc6c5c4)c3)nc2)cc1 ; c1ccc(N(c2cccc(N(c3ccc4c(c3)oc3ccccc34)c3ccc4ccc5cc6sc7ccccc7c6cc5c4c3)c2)c2ccccn2)cc1]

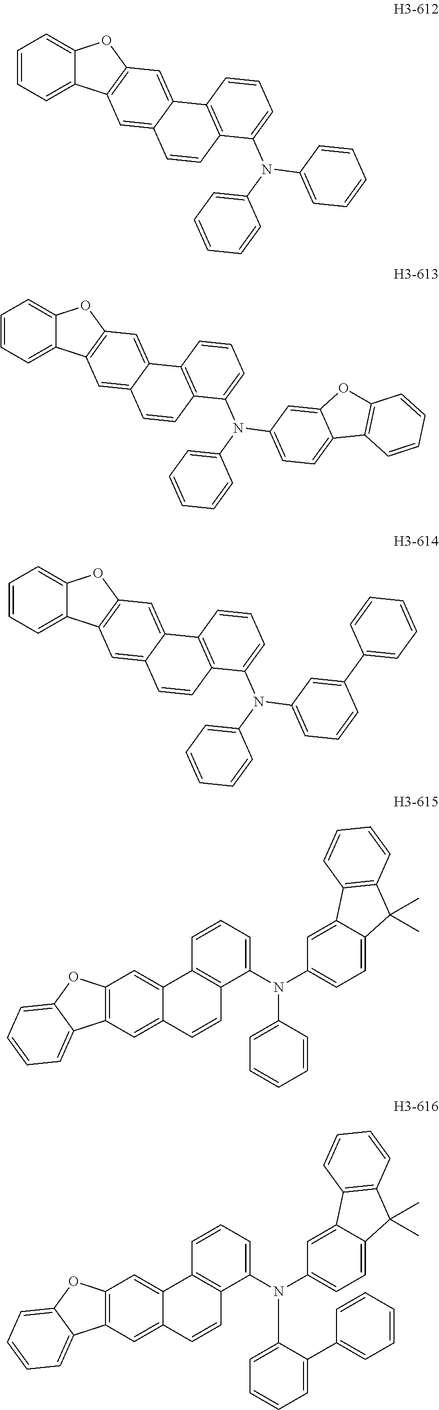 [SMILES: c1ccc(N(c2ccccc2)c2cccc3c2ccc2cc4c(cc23)oc2ccccc24)cc1 ; c1ccc(N(c2ccc3c(c2)oc2ccccc23)c2cccc3c2ccc2cc4c(cc23)oc2ccccc24)cc1 ; c1ccc(-c2cccc(N(c3ccccc3)c3cccc4c3ccc3cc5c(cc34)oc3ccccc35)c2)cc1 ; CC1(C)c2ccccc2-c2cc(N(c3ccccc3)c3cccc4c3ccc3cc5c(cc34)oc3ccccc35)ccc21 ; CC1(C)c2ccccc2-c2cc(N(c3ccccc3-c3ccccc3)c3cccc4c3ccc3cc5c(cc34)oc3ccccc35)ccc21]

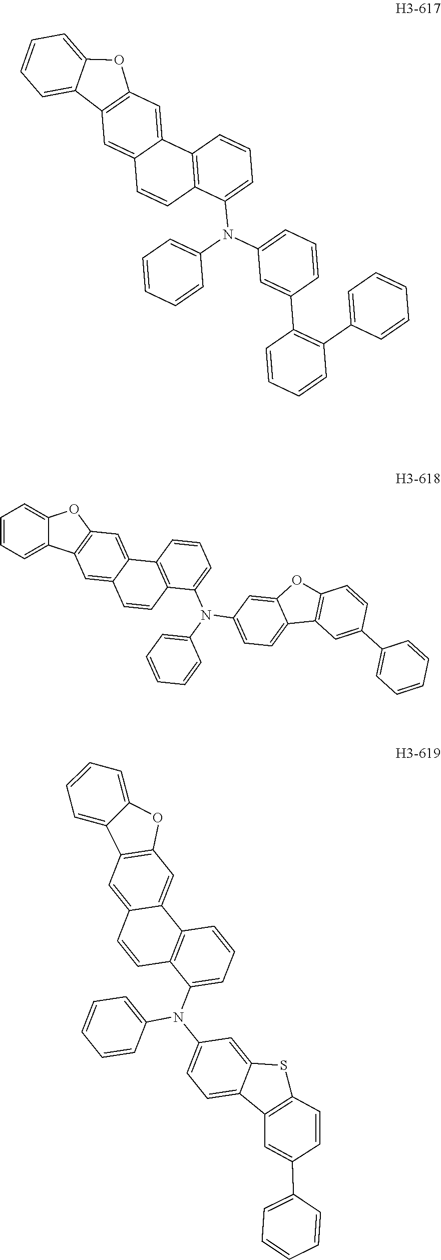 [SMILES: c1ccc(-c2ccccc2-c2cccc(N(c3ccccc3)c3cccc4c3ccc3cc5c(cc34)oc3ccccc35)c2)cc1 ; c1ccc(-c2ccc3oc4cc(N(c5ccccc5)c5cccc6c5ccc5cc7c(cc56)oc5ccccc57)ccc4c3c2)cc1 ; c1ccc(-c2ccc3sc4cc(N(c5ccccc5)c5cccc6c5ccc5cc7c(cc56)oc5ccccc57)ccc4c3c2)cc1]

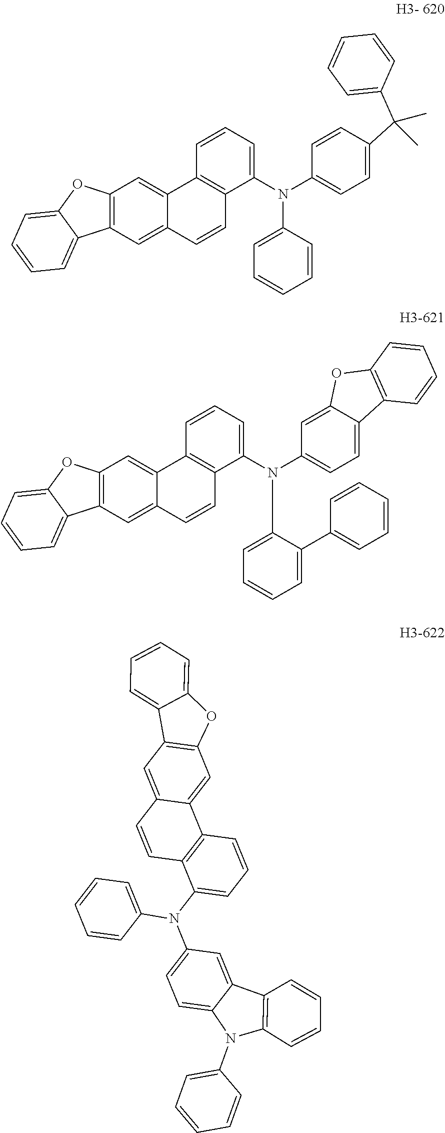 [SMILES: CC(C)(c1ccccc1)c1ccc(N(c2ccccc2)c2cccc3c2ccc2cc4c(cc23)oc2ccccc24)cc1 ; c1ccc(-c2ccccc2N(c2ccc3c(c2)oc2ccccc23)c2cccc3c2ccc2cc4c(cc23)oc2ccccc24)cc1 ; c1ccc(N(c2ccc3c(c2)c2ccccc2n3-c2ccccc2)c2cccc3c2ccc2cc4c(cc23)oc2ccccc24)cc1]

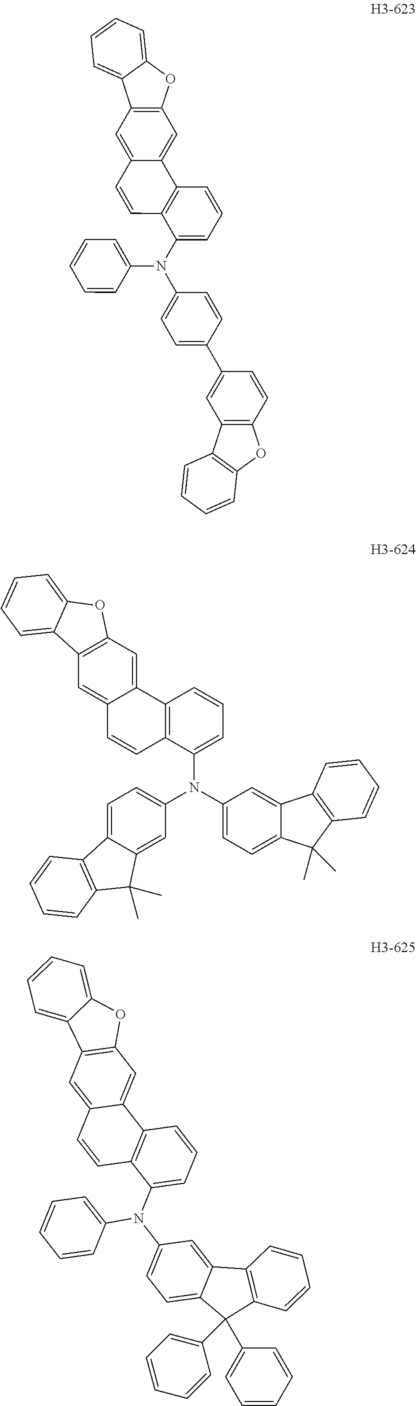

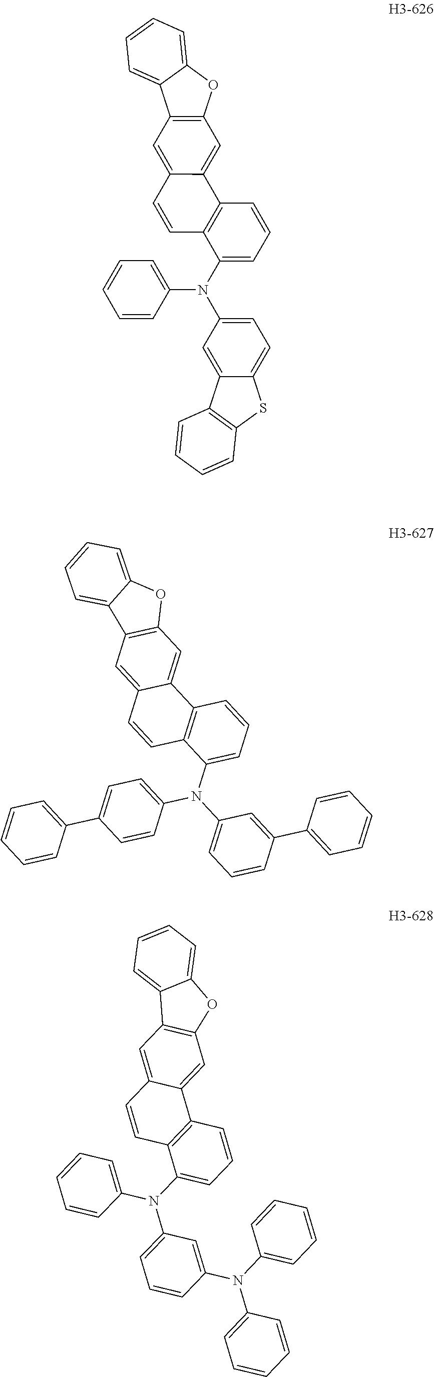 [SMILES: c1ccc(N(c2ccc3sc4ccccc4c3c2)c2cccc3c2ccc2cc4c(cc23)oc2ccccc24)cc1 ; c1ccc(-c2ccc(N(c3cccc(-c4ccccc4)c3)c3cccc4c3ccc3cc5c(cc34)oc3ccccc35)cc2)cc1 ; c1ccc(N(c2ccccc2)c2cccc(N(c3ccccc3)c3cccc4c3ccc3cc5c(cc34)oc3ccccc35)c2)cc1]

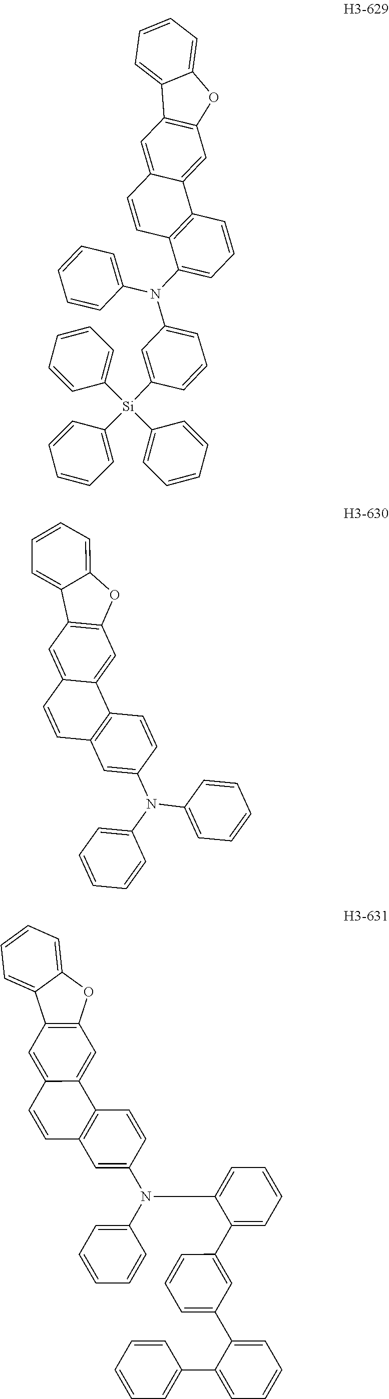

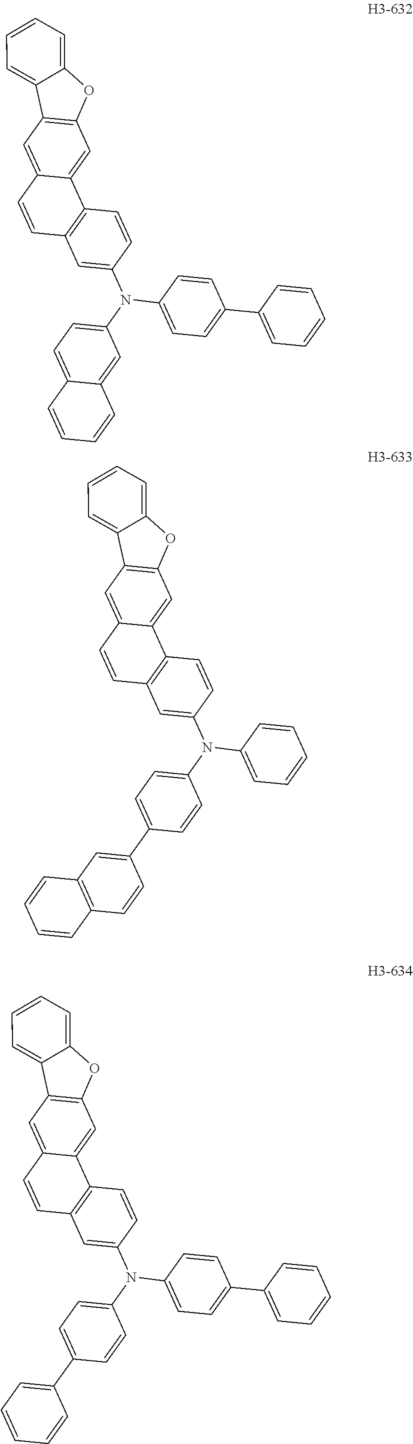

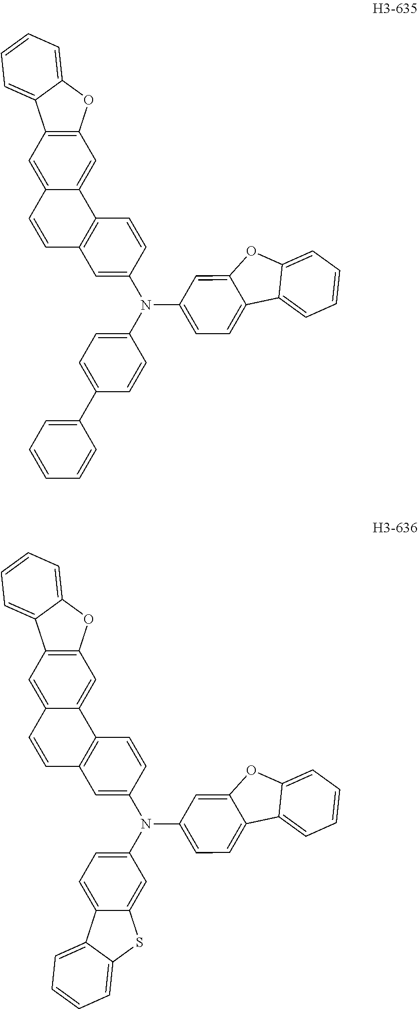 [SMILES: c1ccc(-c2ccc(N(c3ccc4c(ccc5cc6c(cc54)oc4ccccc46)c3)c3ccc4c(c3)oc3ccccc34)cc2)cc1 ; c1ccc2c(c1)oc1cc(N(c3ccc4c(ccc5cc6c(cc54)oc4ccccc46)c3)c3ccc4c(c3)sc3ccccc34)ccc12]

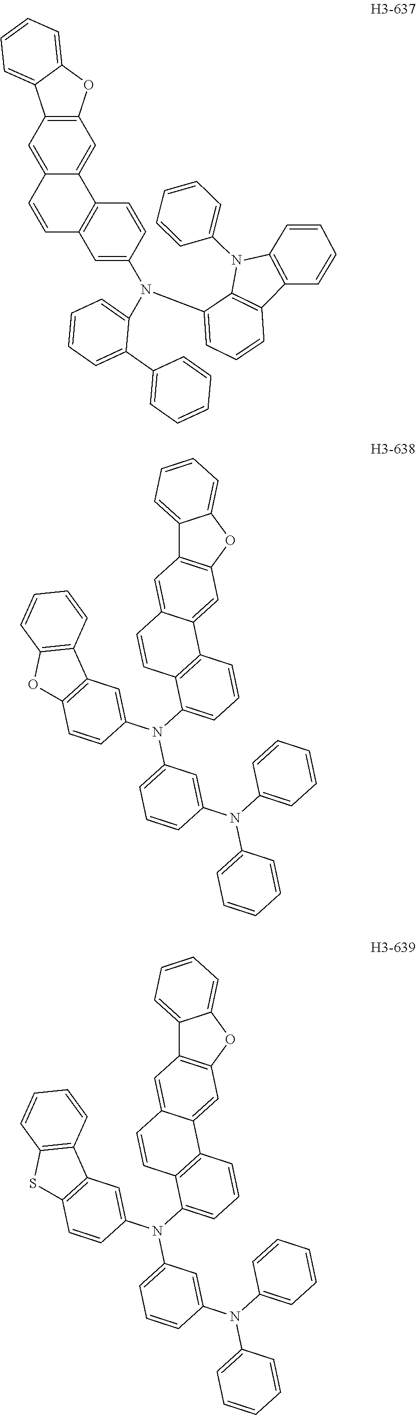

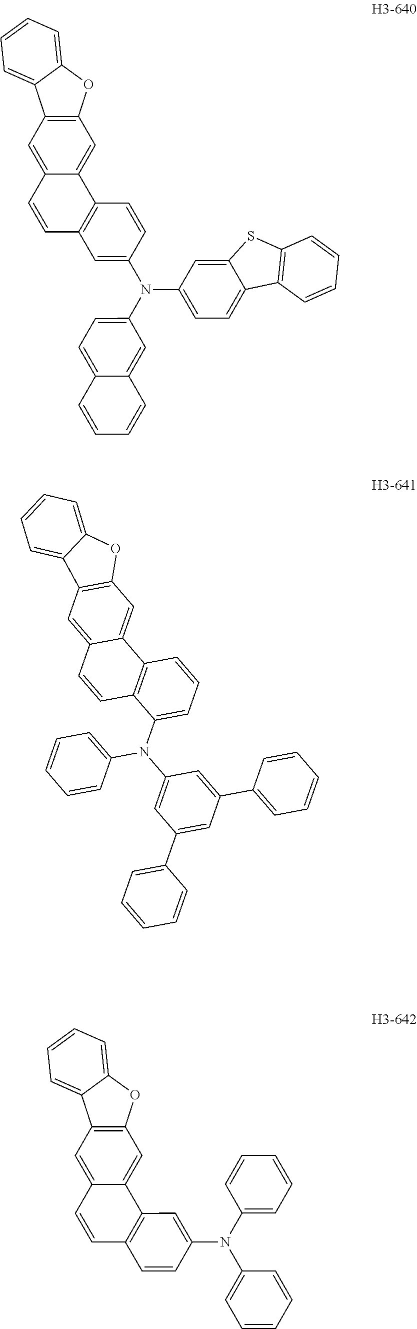 [SMILES: c1ccc2cc(N(c3ccc4c(ccc5cc6c(cc54)oc4ccccc46)c3)c3ccc4c(c3)sc3ccccc34)ccc2c1 ; c1ccc(-c2cc(-c3ccccc3)cc(N(c3ccccc3)c3cccc4c3ccc3cc5c(cc34)oc3ccccc35)c2)cc1 ; c1ccc(N(c2ccccc2)c2ccc3ccc4cc5c(cc4c3c2)oc2ccccc25)cc1]

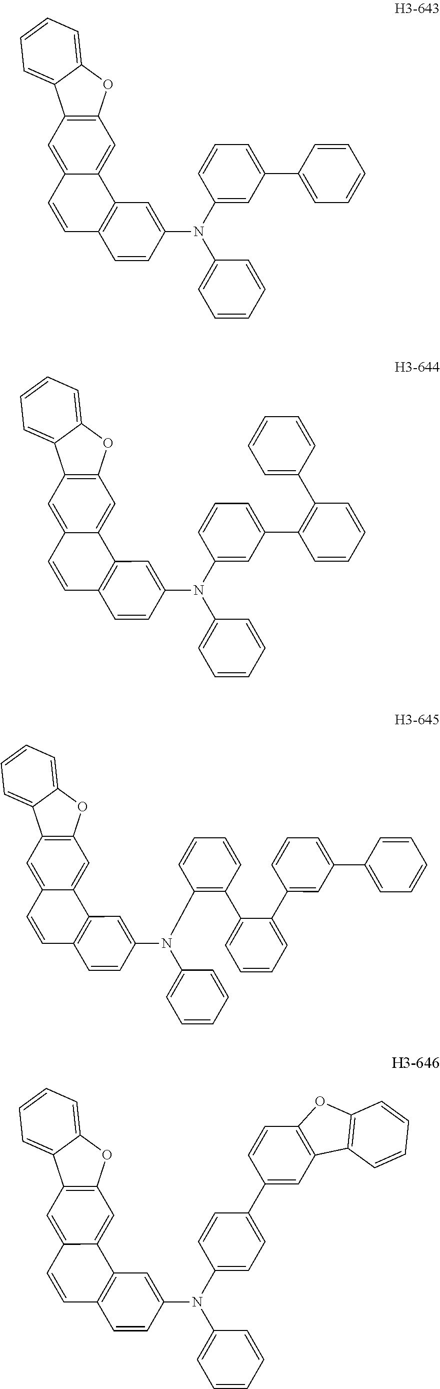 [SMILES: c1ccc(-c2cccc(N(c3ccccc3)c3ccc4ccc5cc6c(cc5c4c3)oc3ccccc36)c2)cc1 ; c1ccc(-c2ccccc2-c2cccc(N(c3ccccc3)c3ccc4ccc5cc6c(cc5c4c3)oc3ccccc36)c2)cc1 ; c1ccc(-c2cccc(-c3ccccc3-c3ccccc3N(c3ccccc3)c3ccc4ccc5cc6c(cc5c4c3)oc3ccccc36)c2)cc1 ; c1ccc(N(c2ccc(-c3ccc4oc5ccccc5c4c3)cc2)c2ccc3ccc4cc5c(cc4c3c2)oc2ccccc25)cc1]

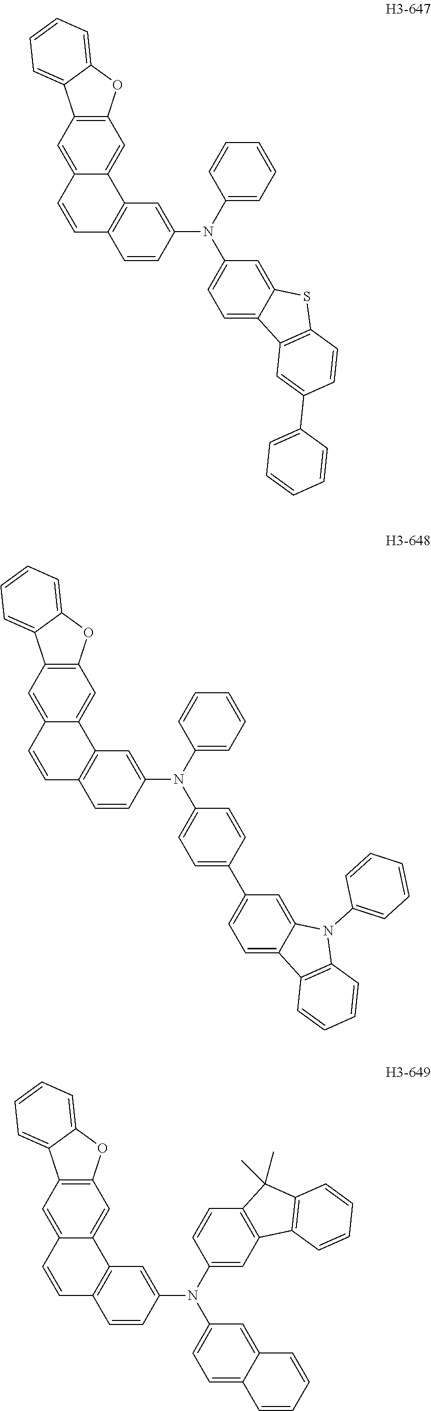 [SMILES: c1ccc(-c2ccc3sc4cc(N(c5ccccc5)c5ccc6ccc7cc8c(cc7c6c5)oc5ccccc58)ccc4c3c2)cc1 ; c1ccc(N(c2ccc(-c3ccc4c5ccccc5n(-c5ccccc5)c4c3)cc2)c2ccc3ccc4cc5c(cc4c3c2)oc2ccccc25)cc1 ; CC1(C)c2ccccc2-c2cc(N(c3ccc4ccccc4c3)c3ccc4ccc5cc6c(cc5c4c3)oc3ccccc36)ccc21]

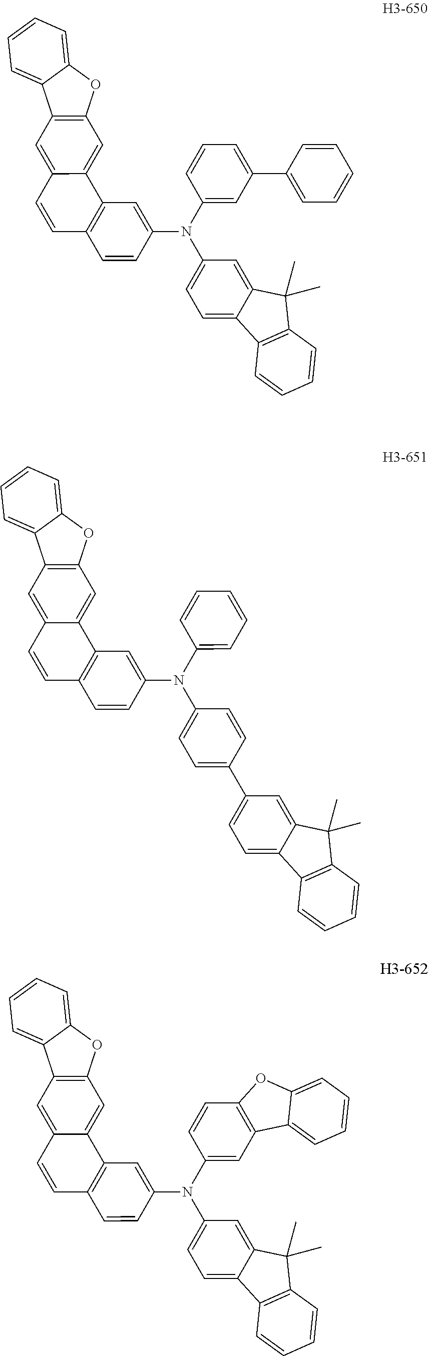 [SMILES: CC1(C)c2ccccc2-c2ccc(N(c3cccc(-c4ccccc4)c3)c3ccc4ccc5cc6c(cc5c4c3)oc3ccccc36)cc21 ; CC1(C)c2ccccc2-c2ccc(-c3ccc(N(c4ccccc4)c4ccc5ccc6cc7c(cc6c5c4)oc4ccccc47)cc3)cc21 ; CC1(C)c2ccccc2-c2ccc(N(c3ccc4ccc5cc6c(cc5c4c3)oc3ccccc36)c3ccc4oc5ccccc5c4c3)cc21]

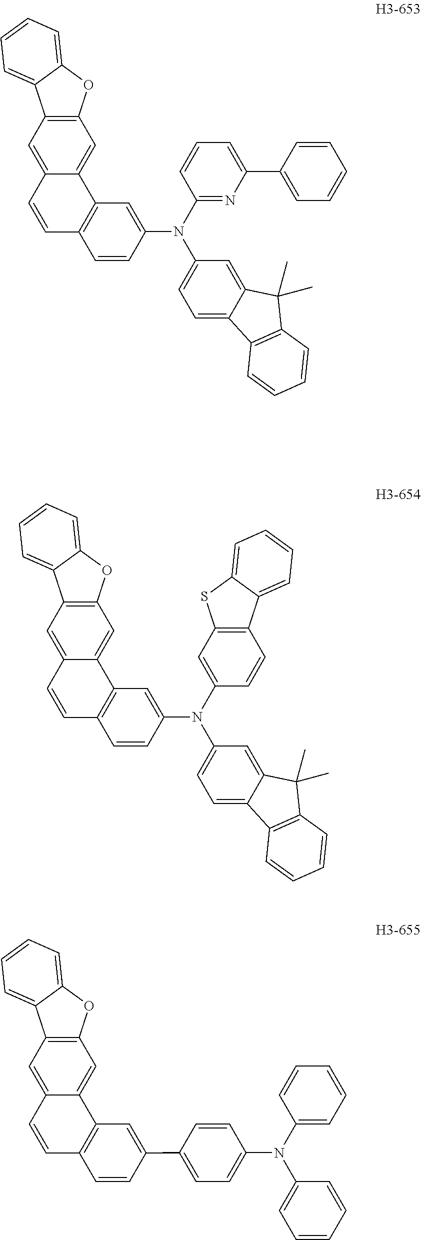 [SMILES: CC1(C)c2ccccc2-c2ccc(N(c3ccc4ccc5cc6c(cc5c4c3)oc3ccccc36)c3cccc(-c4ccccc4)n3)cc21 ; CC1(C)c2ccccc2-c2ccc(N(c3ccc4c(c3)sc3ccccc34)c3ccc4ccc5cc6c(cc5c4c3)oc3ccccc36)cc21 ; c1ccc(N(c2ccccc2)c2ccc(-c3ccc4ccc5cc6c(cc5c4c3)oc3ccccc36)cc2)cc1]

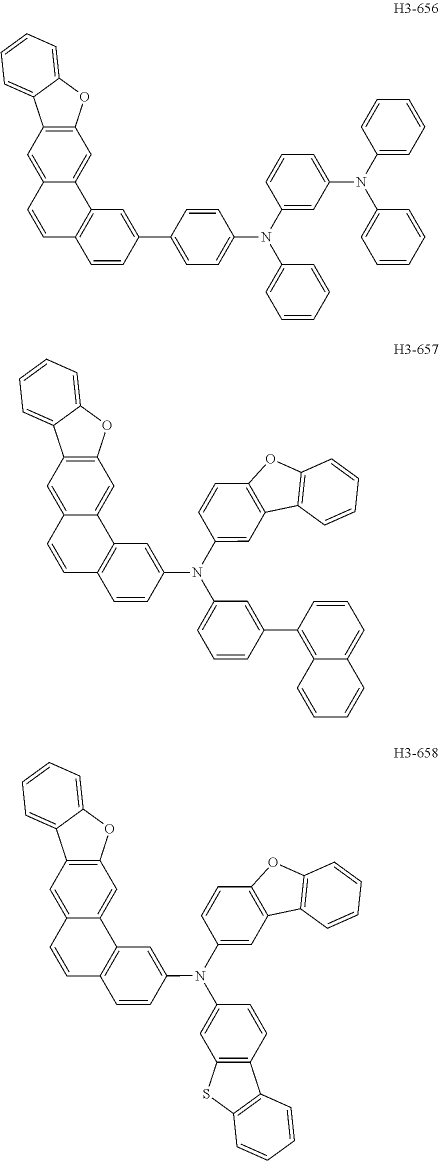 [SMILES: c1ccc(N(c2ccccc2)c2cccc(N(c3ccccc3)c3ccc(-c4ccc5ccc6cc7c(cc6c5c4)oc4ccccc47)cc3)c2)cc1 ; c1cc(-c2cccc3ccccc23)cc(N(c2ccc3ccc4cc5c(cc4c3c2)oc2ccccc25)c2ccc3oc4ccccc4c3c2)c1 ; c1ccc2c(c1)oc1ccc(N(c3ccc4c(c3)sc3ccccc34)c3ccc4ccc5cc6c(cc5c4c3)oc3ccccc36)cc12]

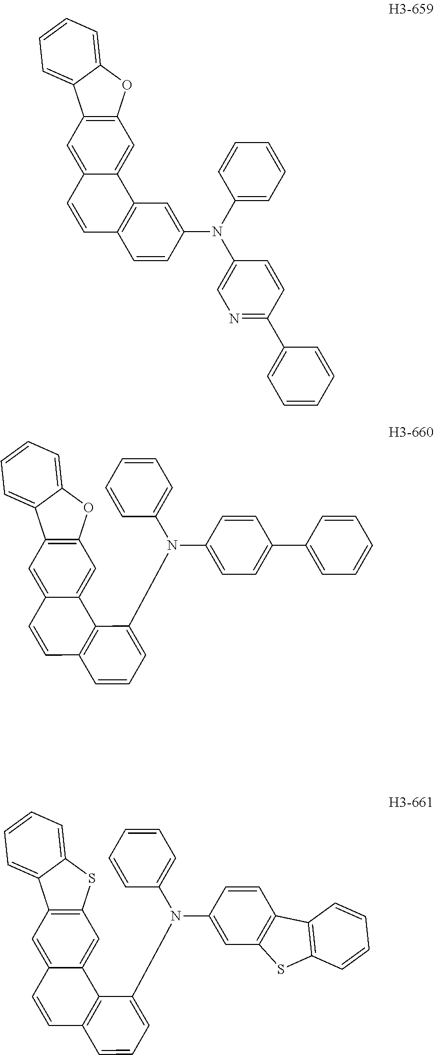 [SMILES: c1ccc(-c2ccc(N(c3ccccc3)c3ccc4ccc5cc6c(cc5c4c3)oc3ccccc36)cn2)cc1 ; c1ccc(-c2ccc(N(c3ccccc3)c3cccc4ccc5cc6c(cc5c34)oc3ccccc36)cc2)cc1 ; c1ccc(N(c2ccc3c(c2)sc2ccccc23)c2cccc3ccc4cc5c(cc4c23)sc2ccccc25)cc1]

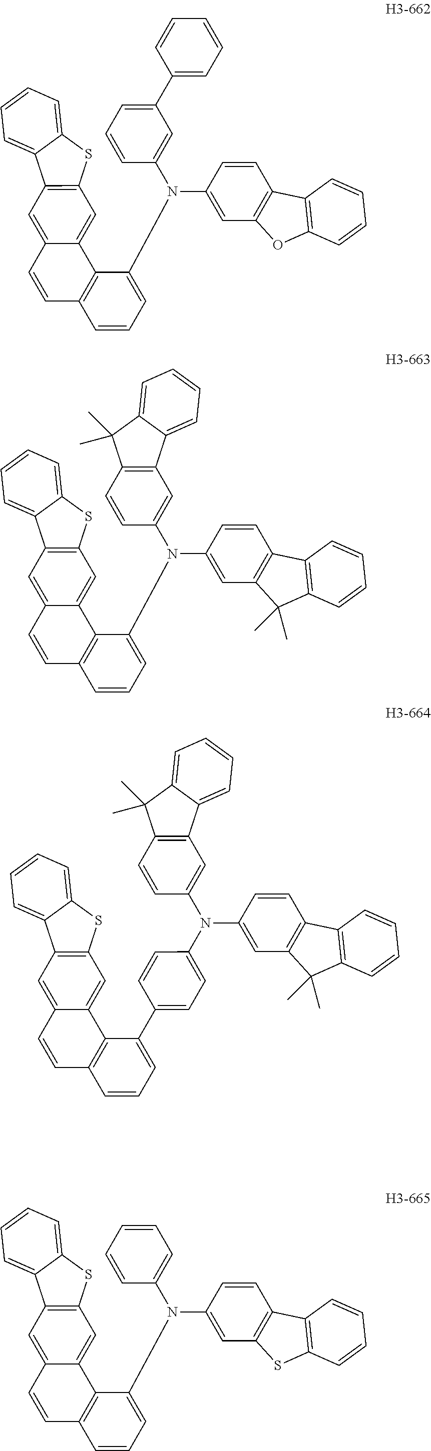 [SMILES: c1ccc(-c2cccc(N(c3ccc4c(c3)oc3ccccc34)c3cccc4ccc5cc6c(cc5c34)sc3ccccc36)c2)cc1 ; CC1(C)c2ccccc2-c2cc(N(c3ccc4c(c3)C(C)(C)c3ccccc3-4)c3cccc4ccc5cc6c(cc5c34)sc3ccccc36)ccc21 ; CC1(C)c2ccccc2-c2cc(N(c3ccc(-c4cccc5ccc6cc7c(cc6c45)sc4ccccc47)cc3)c3ccc4c(c3)C(C)(C)c3ccccc3-4)ccc21 ; c1ccc(N(c2ccc3c(c2)sc2ccccc23)c2cccc3ccc4cc5c(cc4c23)sc2ccccc25)cc1]

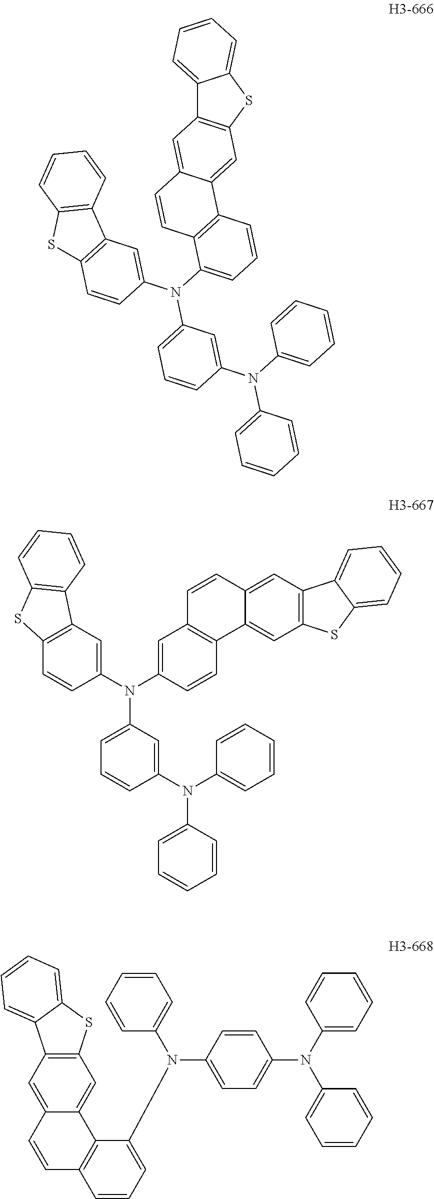 [SMILES: c1ccc(N(c2ccccc2)c2cccc(N(c3ccc4sc5ccccc5c4c3)c3cccc4c3ccc3cc5c(cc34)sc3ccccc35)c2)cc1 ; c1ccc(N(c2ccccc2)c2cccc(N(c3ccc4c(ccc5cc6c(cc54)sc4ccccc46)c3)c3ccc4sc5ccccc5c4c3)c2)cc1 ; c1ccc(N(c2ccccc2)c2ccc(N(c3ccccc3)c3cccc4ccc5cc6c(cc5c34)sc3ccccc36)cc2)cc1]

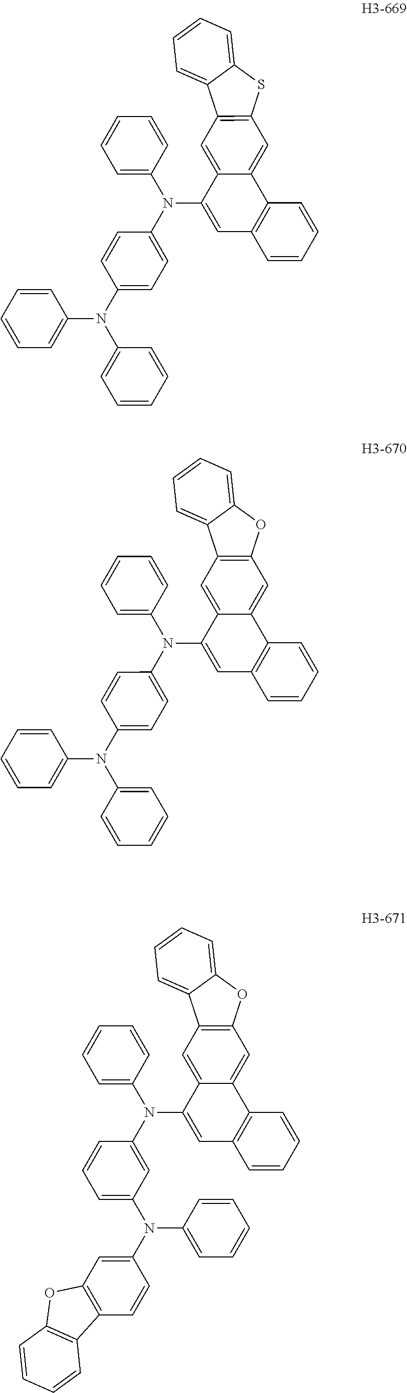 [SMILES: c1ccc(N(c2ccccc2)c2ccc(N(c3ccccc3)c3cc4ccccc4c4cc5sc6ccccc6c5cc34)cc2)cc1 ; c1ccc(N(c2ccccc2)c2ccc(N(c3ccccc3)c3cc4ccccc4c4cc5oc6ccccc6c5cc34)cc2)cc1 ; c1ccc(N(c2cccc(N(c3ccccc3)c3cc4ccccc4c4cc5oc6ccccc6c5cc34)c2)c2ccc3c(c2)oc2ccccc23)cc1]

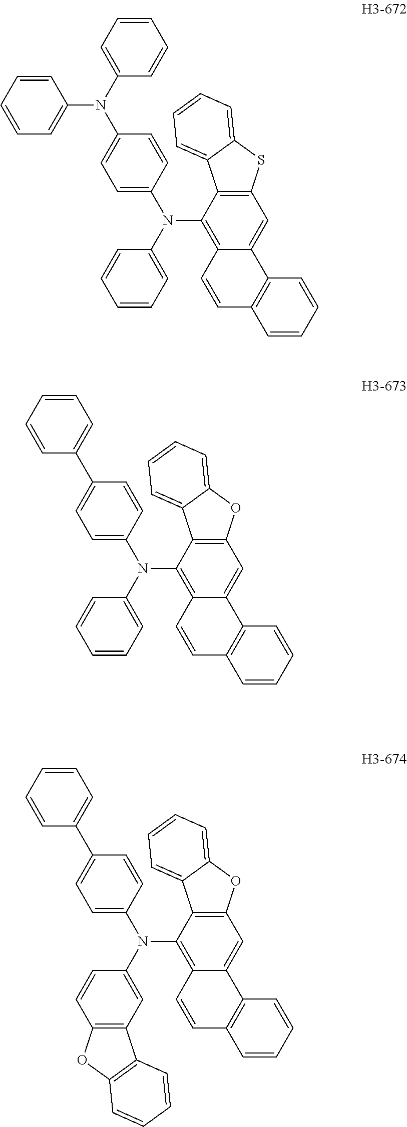 [SMILES: c1ccc(N(c2ccccc2)c2ccc(N(c3ccccc3)c3c4ccc5ccccc5c4cc4sc5ccccc5c34)cc2)cc1 ; c1ccc(-c2ccc(N(c3ccccc3)c3c4ccc5ccccc5c4cc4oc5ccccc5c34)cc2)cc1 ; c1ccc(-c2ccc(N(c3ccc4oc5ccccc5c4c3)c3c4ccc5ccccc5c4cc4oc5ccccc5c34)cc2)cc1]

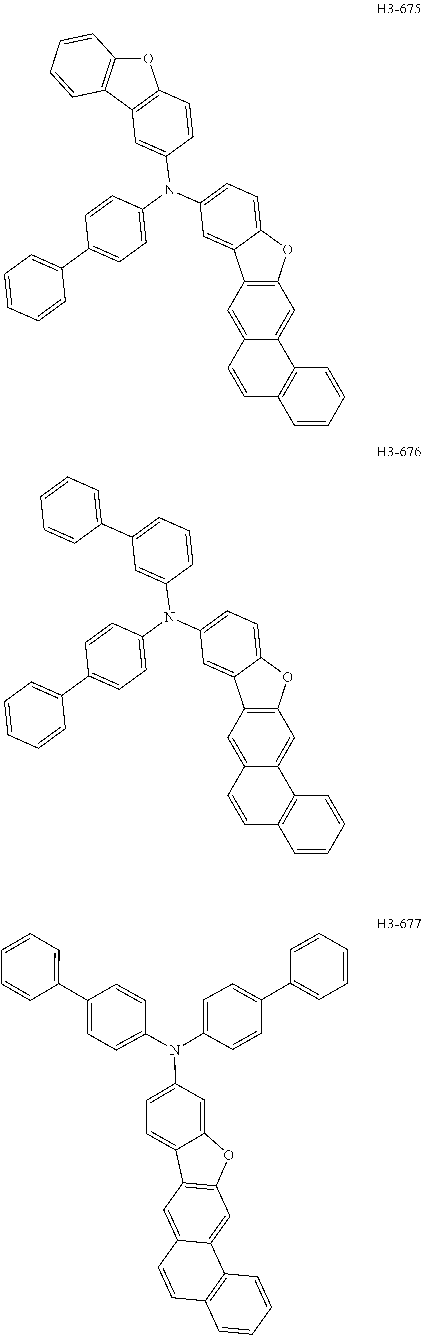 [SMILES: c1ccc(-c2ccc(N(c3ccc4oc5ccccc5c4c3)c3ccc4oc5cc6c(ccc7ccccc76)cc5c4c3)cc2)cc1 ; c1ccc(-c2ccc(N(c3cccc(-c4ccccc4)c3)c3ccc4oc5cc6c(ccc7ccccc76)cc5c4c3)cc2)cc1 ; c1ccc(-c2ccc(N(c3ccc(-c4ccccc4)cc3)c3ccc4c(c3)oc3cc5c(ccc6ccccc65)cc34)cc2)cc1]

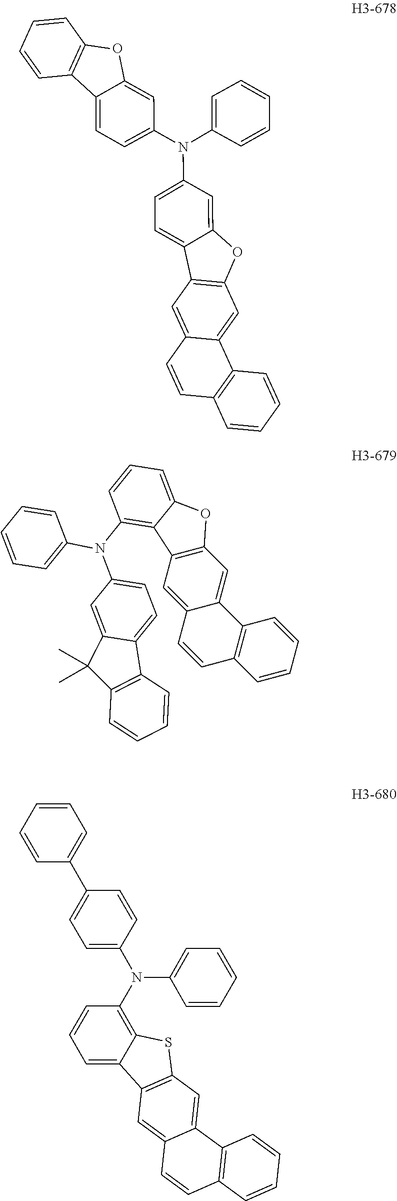 [SMILES: c1ccc(N(c2ccc3c(c2)oc2ccccc23)c2ccc3c(c2)oc2cc4c(ccc5ccccc54)cc23)cc1 ; CC1(C)c2ccccc2-c2ccc(N(c3ccccc3)c3cccc4oc5cc6c(ccc7ccccc76)cc5c34)cc21 ; c1ccc(-c2ccc(N(c3ccccc3)c3cccc4c3sc3cc5c(ccc6ccccc65)cc34)cc2)cc1]

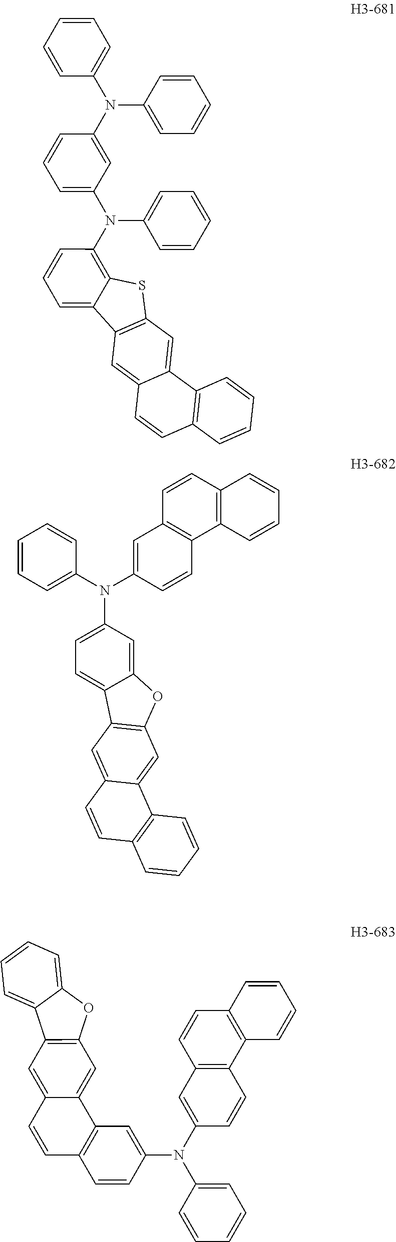

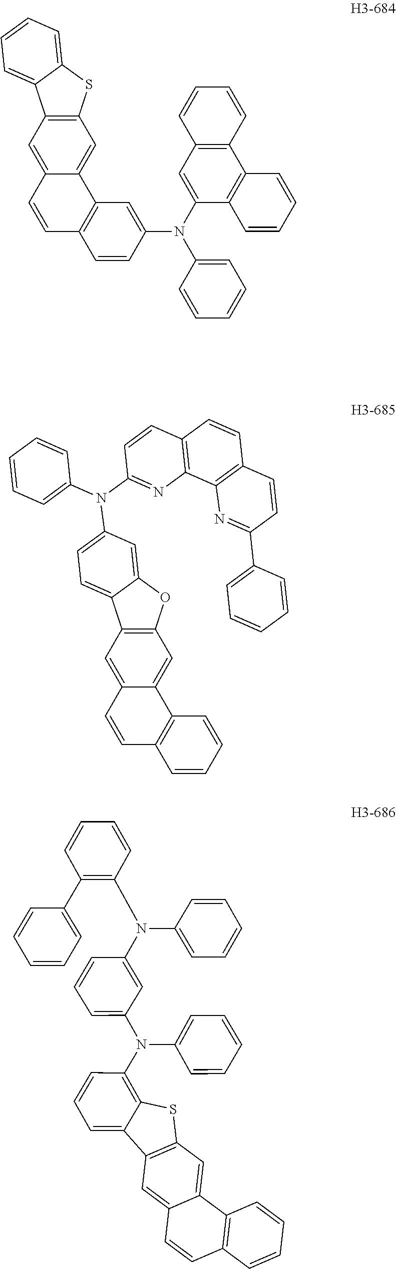 [SMILES: c1ccc(N(c2ccc3ccc4cc5c(cc4c3c2)sc2ccccc25)c2cc3ccccc3c3ccccc23)cc1 ; c1ccc(-c2ccc3ccc4ccc(N(c5ccccc5)c5ccc6c(c5)oc5cc7c(ccc8ccccc87)cc56)nc4c3n2)cc1 ; c1ccc(-c2ccccc2N(c2ccccc2)c2cccc(N(c3ccccc3)c3cccc4c3sc3cc5c(ccc6ccccc65)cc34)c2)cc1]

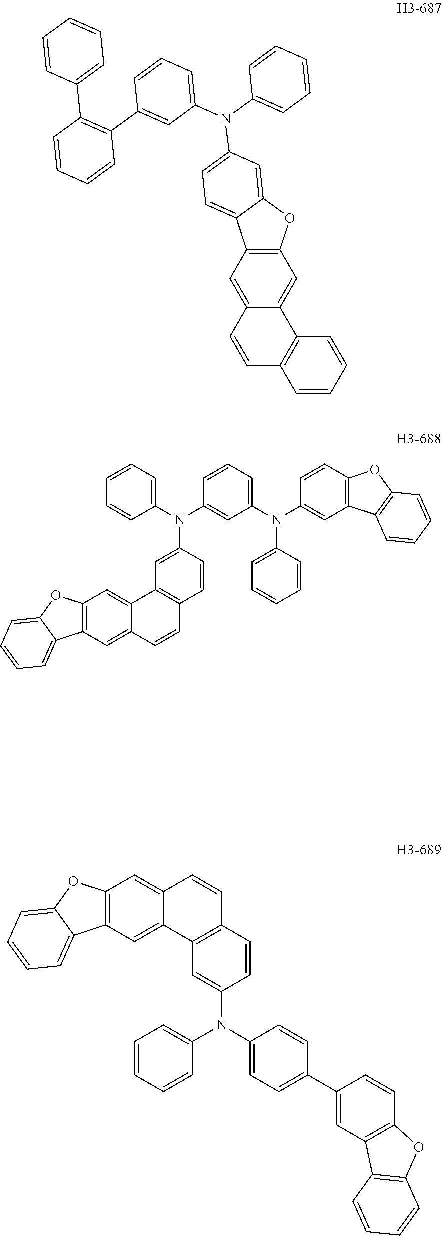 [SMILES: c1ccc(-c2ccccc2-c2cccc(N(c3ccccc3)c3ccc4c(c3)oc3cc5c(ccc6ccccc65)cc34)c2)cc1 ; c1ccc(N(c2cccc(N(c3ccccc3)c3ccc4oc5ccccc5c4c3)c2)c2ccc3ccc4cc5c(cc4c3c2)oc2ccccc25)cc1 ; c1ccc(N(c2ccc(-c3ccc4oc5ccccc5c4c3)cc2)c2ccc3ccc4cc5oc6ccccc6c5cc4c3c2)cc1]

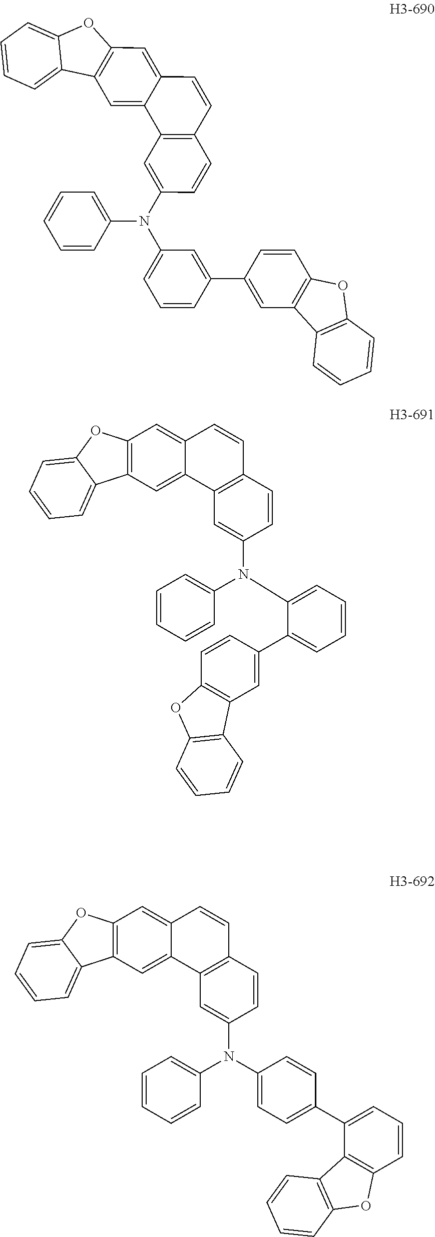 [SMILES: c1ccc(N(c2cccc(-c3ccc4oc5ccccc5c4c3)c2)c2ccc3ccc4cc5oc6ccccc6c5cc4c3c2)cc1 ; c1ccc(N(c2ccc3ccc4cc5oc6ccccc6c5cc4c3c2)c2ccccc2-c2ccc3oc4ccccc4c3c2)cc1 ; c1ccc(N(c2ccc(-c3cccc4oc5ccccc5c34)cc2)c2ccc3ccc4cc5oc6ccccc6c5cc4c3c2)cc1]

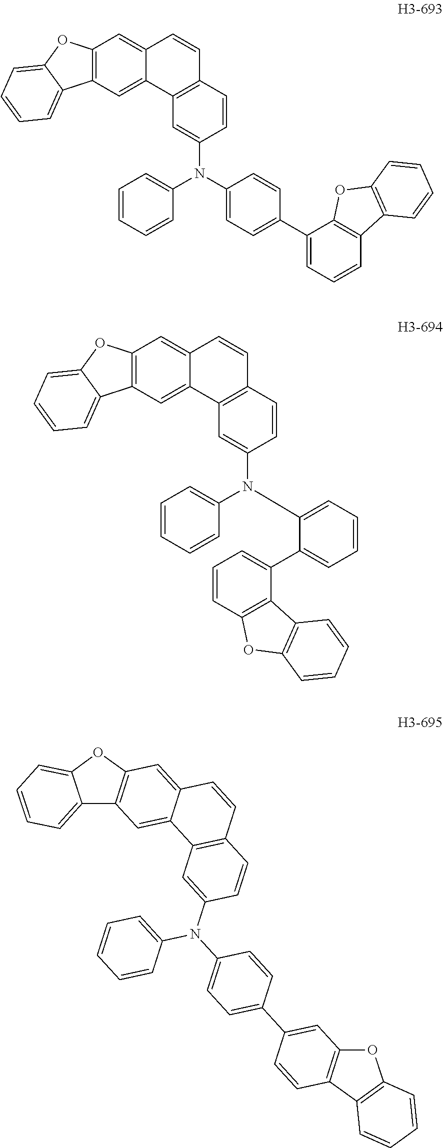 [SMILES: c1ccc(N(c2ccc(-c3cccc4c3oc3ccccc34)cc2)c2ccc3ccc4cc5oc6ccccc6c5cc4c3c2)cc1 ; c1ccc(N(c2ccc3ccc4cc5oc6ccccc6c5cc4c3c2)c2ccccc2-c2cccc3oc4ccccc4c23)cc1 ; c1ccc(N(c2ccc(-c3ccc4c(c3)oc3ccccc34)cc2)c2ccc3ccc4cc5oc6ccccc6c5cc4c3c2)cc1]

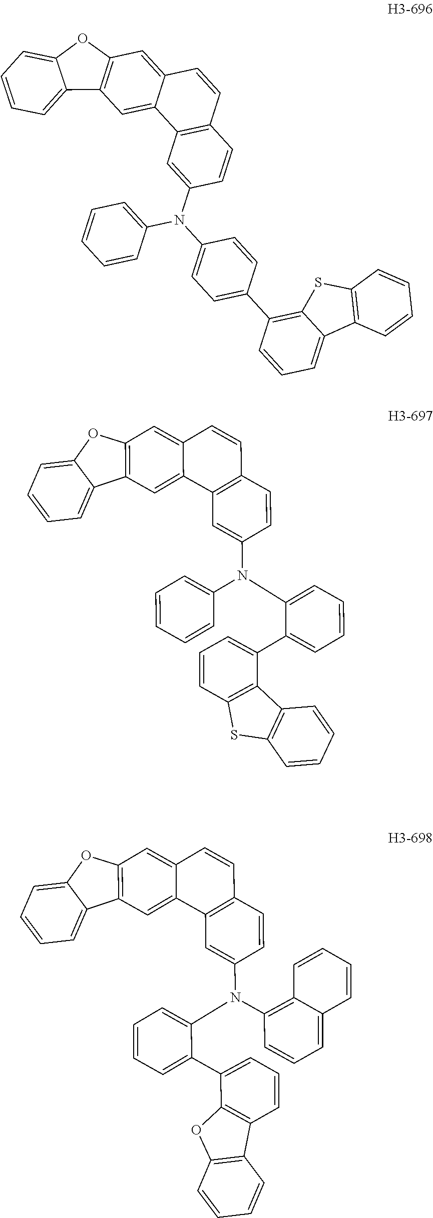 [SMILES: c1ccc(N(c2ccc(-c3cccc4c3sc3ccccc34)cc2)c2ccc3ccc4cc5oc6ccccc6c5cc4c3c2)cc1 ; c1ccc(N(c2ccc3ccc4cc5oc6ccccc6c5cc4c3c2)c2ccccc2-c2cccc3sc4ccccc4c23)cc1 ; c1ccc(N(c2ccc3ccc4cc5oc6ccccc6c5cc4c3c2)c2cccc3ccccc23)c(-c2cccc3c2oc2ccccc23)c1]

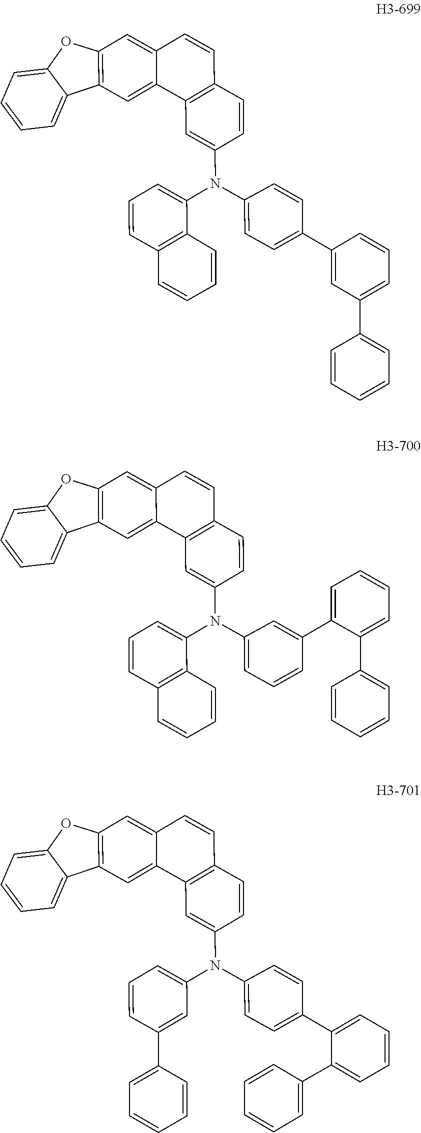 [SMILES: c1ccc(-c2cccc(-c3ccc(N(c4ccc5ccc6cc7oc8ccccc8c7cc6c5c4)c4cccc5ccccc45)cc3)c2)cc1 ; c1ccc(-c2ccccc2-c2cccc(N(c3ccc4ccc5cc6oc7ccccc7c6cc5c4c3)c3cccc4ccccc34)c2)cc1 ; c1ccc(-c2cccc(N(c3ccc(-c4ccccc4-c4ccccc4)cc3)c3ccc4ccc5cc6oc7ccccc7c6cc5c4c3)c2)cc1]

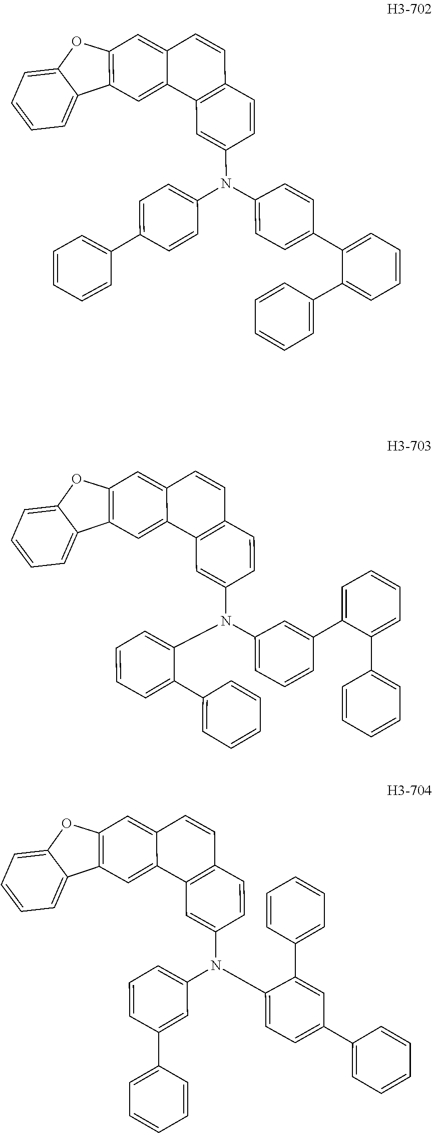 [SMILES: c1ccc(-c2ccc(N(c3ccc(-c4ccccc4-c4ccccc4)cc3)c3ccc4ccc5cc6oc7ccccc7c6cc5c4c3)cc2)cc1 ; c1ccc(-c2ccccc2-c2cccc(N(c3ccc4ccc5cc6oc7ccccc7c6cc5c4c3)c3ccccc3-c3ccccc3)c2)cc1 ; c1ccc(-c2cccc(N(c3ccc4ccc5cc6oc7ccccc7c6cc5c4c3)c3ccc(-c4ccccc4)cc3-c3ccccc3)c2)cc1]

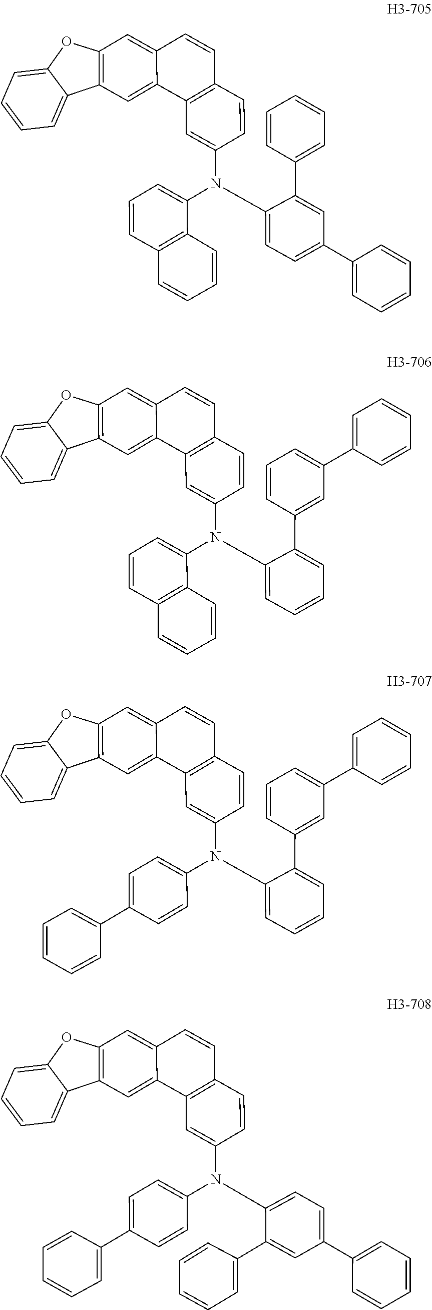 [SMILES: c1ccc(-c2ccc(N(c3ccc4ccc5cc6oc7ccccc7c6cc5c4c3)c3cccc4ccccc34)c(-c3ccccc3)c2)cc1 ; c1ccc(-c2cccc(-c3ccccc3N(c3ccc4ccc5cc6oc7ccccc7c6cc5c4c3)c3cccc4ccccc34)c2)cc1 ; c1ccc(-c2ccc(N(c3ccc4ccc5cc6oc7ccccc7c6cc5c4c3)c3ccccc3-c3cccc(-c4ccccc4)c3)cc2)cc1 ; c1ccc(-c2ccc(N(c3ccc4ccc5cc6oc7ccccc7c6cc5c4c3)c3ccc(-c4ccccc4)cc3-c3ccccc3)cc2)cc1]

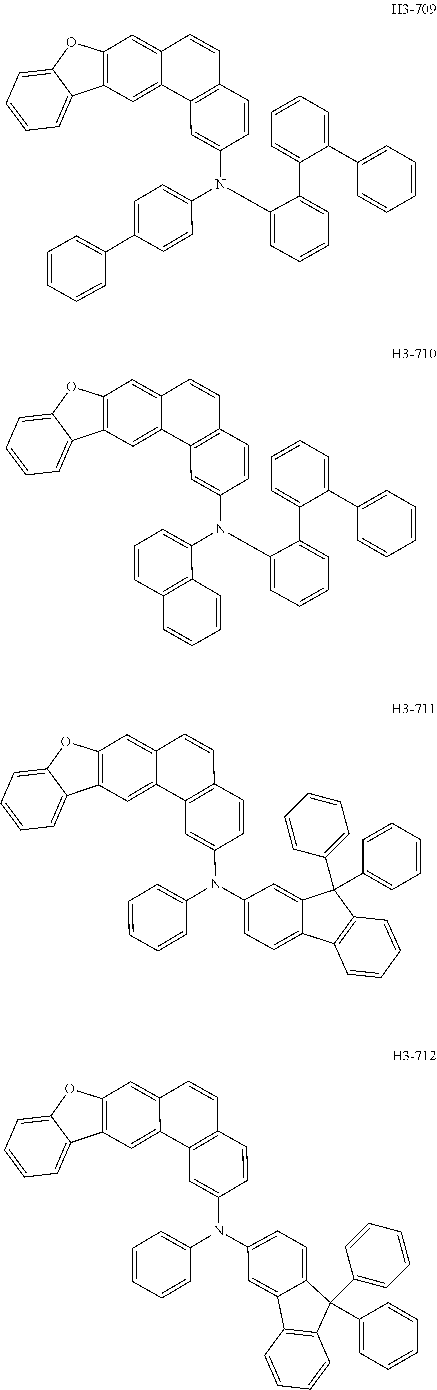 [SMILES: c1ccc(-c2ccc(N(c3ccc4ccc5cc6oc7ccccc7c6cc5c4c3)c3ccccc3-c3ccccc3-c3ccccc3)cc2)cc1 ; c1ccc(-c2ccccc2-c2ccccc2N(c2ccc3ccc4cc5oc6ccccc6c5cc4c3c2)c2cccc3ccccc23)cc1 ; c1ccc(N(c2ccc3c(c2)C(c2ccccc2)(c2ccccc2)c2ccccc2-3)c2ccc3ccc4cc5oc6ccccc6c5cc4c3c2)cc1 ; c1ccc(N(c2ccc3c(c2)-c2ccccc2C3(c2ccccc2)c2ccccc2)c2ccc3ccc4cc5oc6ccccc6c5cc4c3c2)cc1]

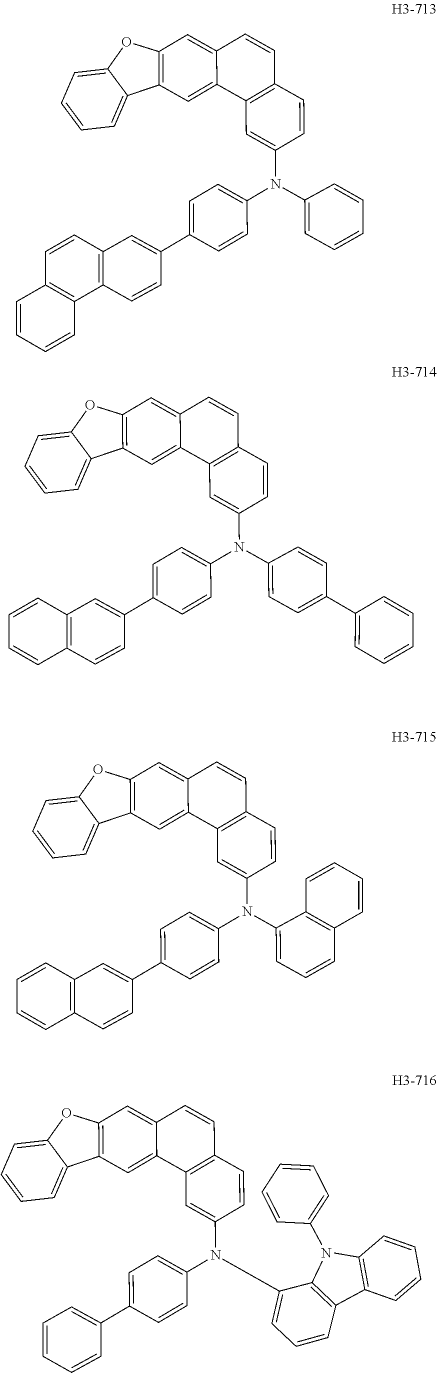 [SMILES: c1ccc(N(c2ccc(-c3ccc4c(ccc5ccccc54)c3)cc2)c2ccc3ccc4cc5oc6ccccc6c5cc4c3c2)cc1 ; c1ccc(-c2ccc(N(c3ccc(-c4ccc5ccccc5c4)cc3)c3ccc4ccc5cc6oc7ccccc7c6cc5c4c3)cc2)cc1 ; c1ccc2cc(-c3ccc(N(c4ccc5ccc6cc7oc8ccccc8c7cc6c5c4)c4cccc5ccccc45)cc3)ccc2c1 ; c1ccc(-c2ccc(N(c3ccc4ccc5cc6oc7ccccc7c6cc5c4c3)c3cccc4c5ccccc5n(-c5ccccc5)c34)cc2)cc1]

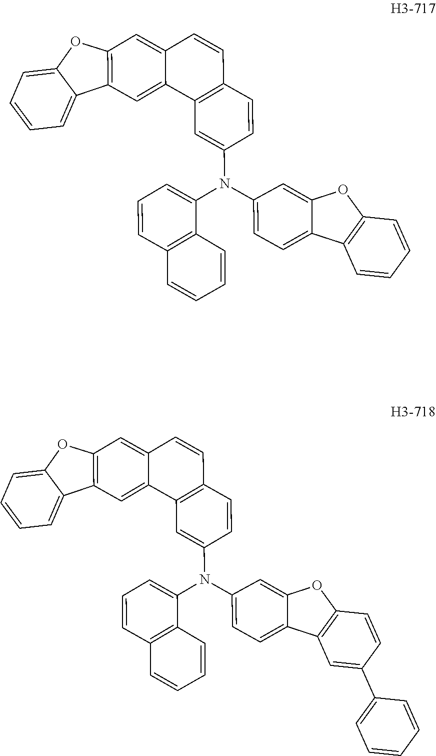 [SMILES: c1ccc2c(N(c3ccc4c(c3)oc3ccccc34)c3ccc4ccc5cc6oc7ccccc7c6cc5c4c3)cccc2c1 ; c1ccc(-c2ccc3oc4cc(N(c5ccc6ccc7cc8oc9ccccc9c8cc7c6c5)c5cccc6ccccc56)ccc4c3c2)cc1]

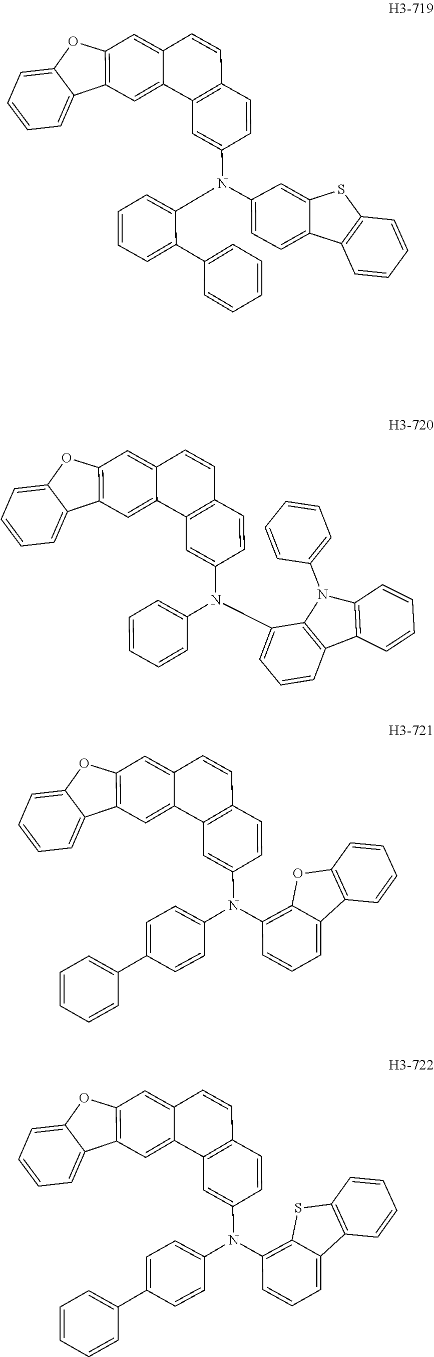 [SMILES: c1ccc(-c2ccccc2N(c2ccc3c(c2)sc2ccccc23)c2ccc3ccc4cc5oc6ccccc6c5cc4c3c2)cc1 ; c1ccc(N(c2ccc3ccc4cc5oc6ccccc6c5cc4c3c2)c2cccc3c4ccccc4n(-c4ccccc4)c23)cc1 ; c1ccc(-c2ccc(N(c3ccc4ccc5cc6oc7ccccc7c6cc5c4c3)c3cccc4c3oc3ccccc34)cc2)cc1 ; c1ccc(-c2ccc(N(c3ccc4ccc5cc6oc7ccccc7c6cc5c4c3)c3cccc4c3sc3ccccc34)cc2)cc1]

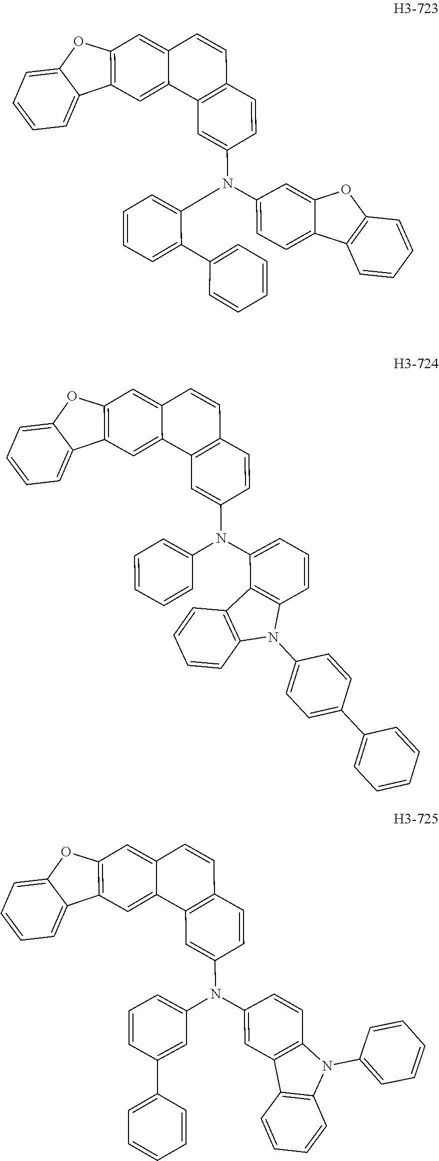 [SMILES: c1ccc(-c2ccccc2N(c2ccc3c(c2)oc2ccccc23)c2ccc3ccc4cc5oc6ccccc6c5cc4c3c2)cc1 ; c1ccc(-c2ccc(-n3c4ccccc4c4c(N(c5ccccc5)c5ccc6ccc7cc8oc9ccccc9c8cc7c6c5)cccc43)cc2)cc1 ; c1ccc(-c2cccc(N(c3ccc4ccc5cc6oc7ccccc7c6cc5c4c3)c3ccc4c(c3)c3ccccc3n4-c3ccccc3)c2)cc1]

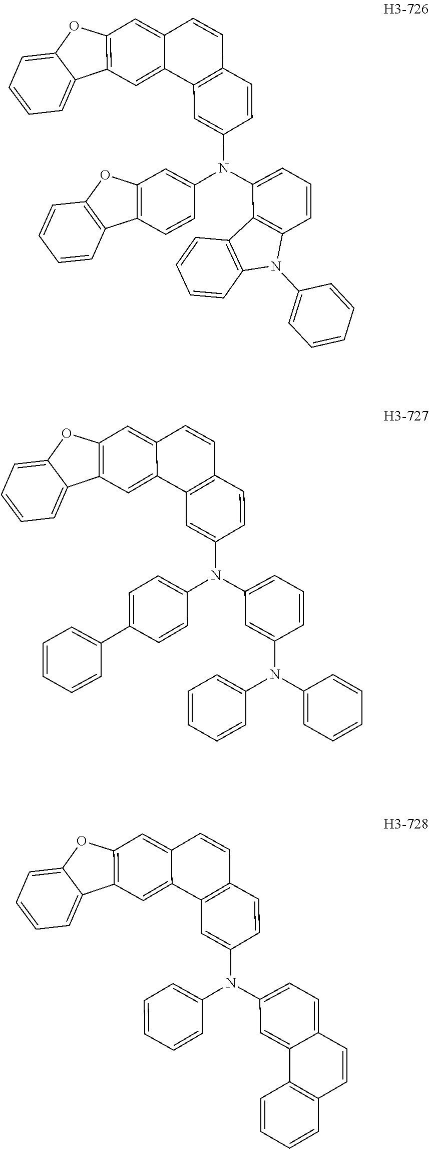 [SMILES: c1ccc(-n2c3ccccc3c3c(N(c4ccc5c(c4)oc4ccccc45)c4ccc5ccc6cc7oc8ccccc8c7cc6c5c4)cccc32)cc1 ; c1ccc(-c2ccc(N(c3cccc(N(c4ccccc4)c4ccccc4)c3)c3ccc4ccc5cc6oc7ccccc7c6cc5c4c3)cc2)cc1 ; c1ccc(N(c2ccc3ccc4ccccc4c3c2)c2ccc3ccc4cc5oc6ccccc6c5cc4c3c2)cc1]

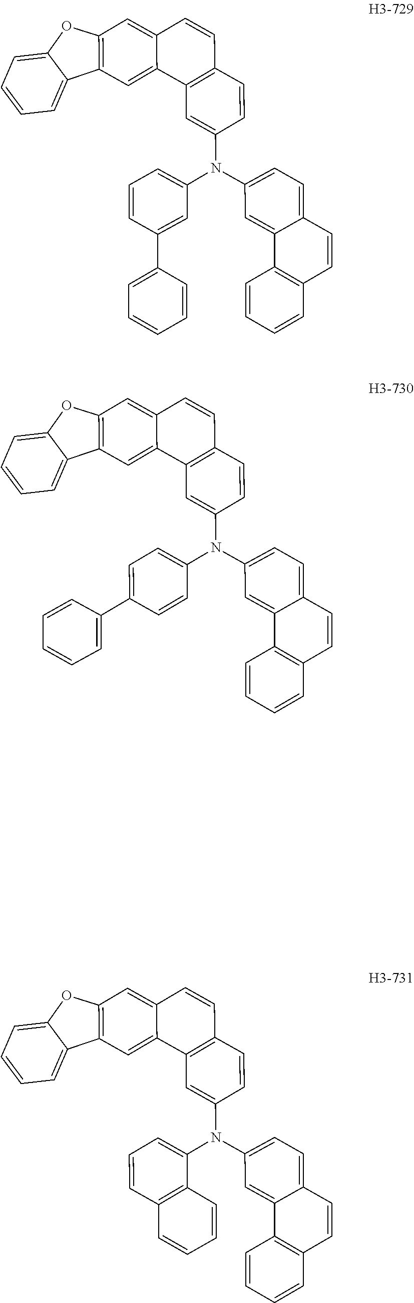 [SMILES: c1ccc(-c2cccc(N(c3ccc4ccc5ccccc5c4c3)c3ccc4ccc5cc6oc7ccccc7c6cc5c4c3)c2)cc1 ; c1ccc(-c2ccc(N(c3ccc4ccc5ccccc5c4c3)c3ccc4ccc5cc6oc7ccccc7c6cc5c4c3)cc2)cc1 ; c1ccc2c(N(c3ccc4ccc5ccccc5c4c3)c3ccc4ccc5cc6oc7ccccc7c6cc5c4c3)cccc2c1]

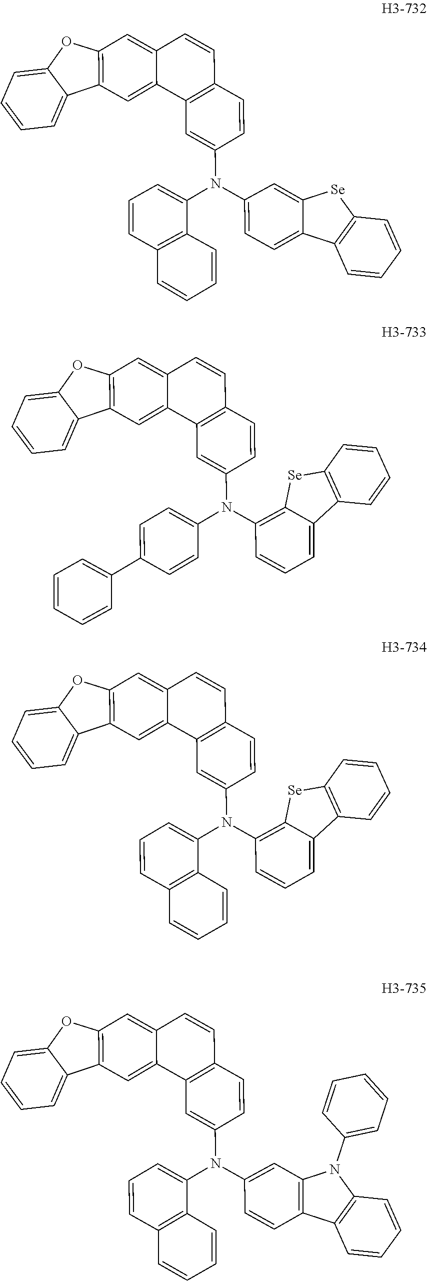 [SMILES: c1ccc2c(N(c3ccc4c(c3)[se]c3ccccc34)c3ccc4ccc5cc6oc7ccccc7c6cc5c4c3)cccc2c1 ; c1ccc(-c2ccc(N(c3ccc4ccc5cc6oc7ccccc7c6cc5c4c3)c3cccc4c3[se]c3ccccc34)cc2)cc1 ; c1ccc2c(N(c3ccc4ccc5cc6oc7ccccc7c6cc5c4c3)c3cccc4c3[se]c3ccccc34)cccc2c1 ; c1ccc(-n2c3ccccc3c3ccc(N(c4ccc5ccc6cc7oc8ccccc8c7cc6c5c4)c4cccc5ccccc45)cc32)cc1]

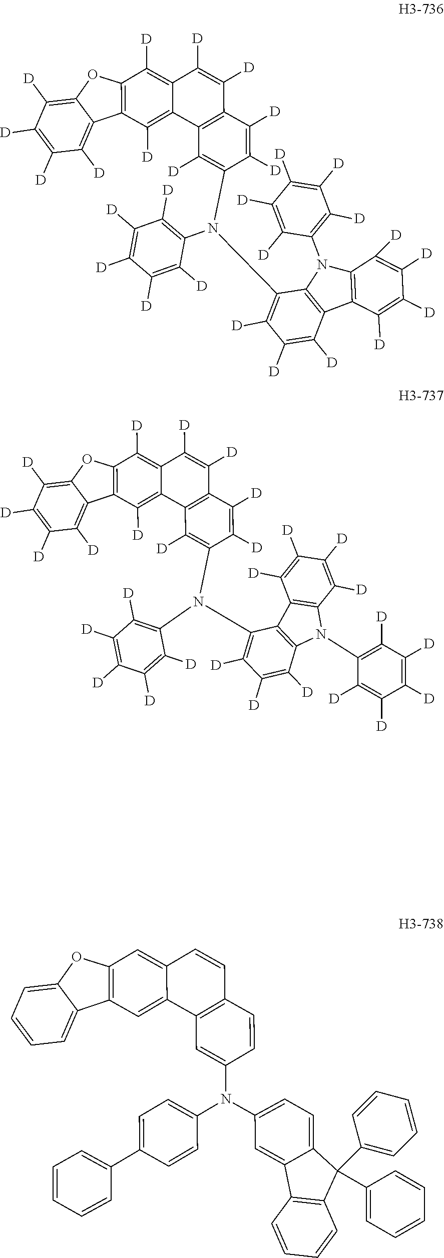

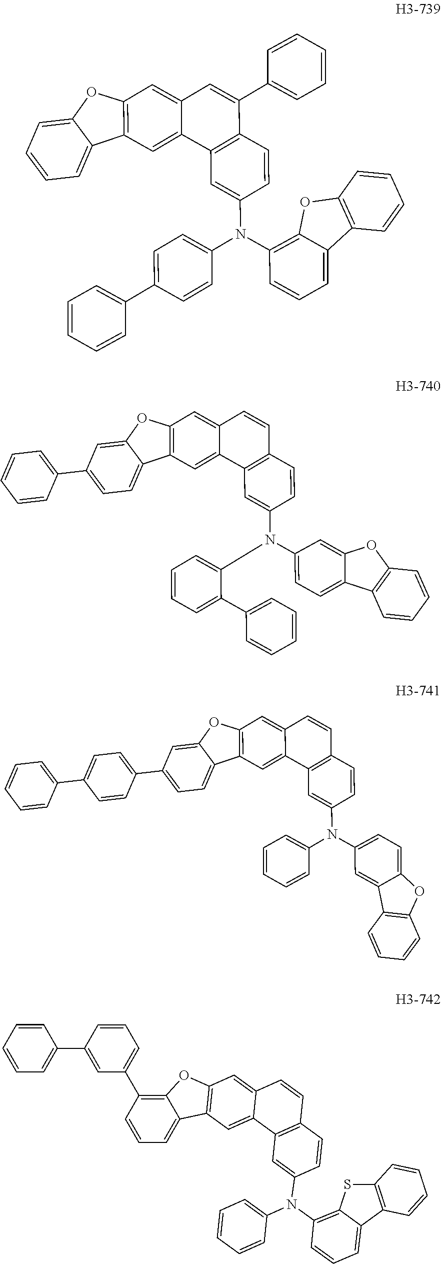 [SMILES: c1ccc(-c2ccc(N(c3ccc4c(-c5ccccc5)cc5cc6oc7ccccc7c6cc5c4c3)c3cccc4c3oc3ccccc34)cc2)cc1 ; c1ccc(-c2ccc3c(c2)oc2cc4ccc5ccc(N(c6ccc7c(c6)oc6ccccc67)c6ccccc6-c6ccccc6)cc5c4cc23)cc1 ; c1ccc(-c2ccc(-c3ccc4c(c3)oc3cc5ccc6ccc(N(c7ccccc7)c7ccc8oc9ccccc9c8c7)cc6c5cc34)cc2)cc1 ; c1ccc(-c2cccc(-c3cccc4c3oc3cc5ccc6ccc(N(c7ccccc7)c7cccc8c7sc7ccccc78)cc6c5cc34)c2)cc1]

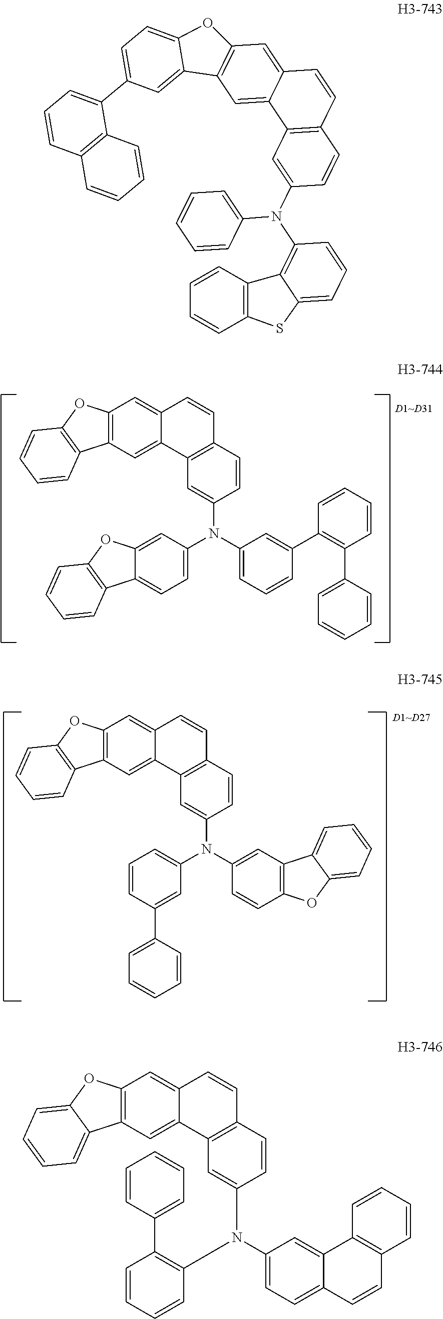

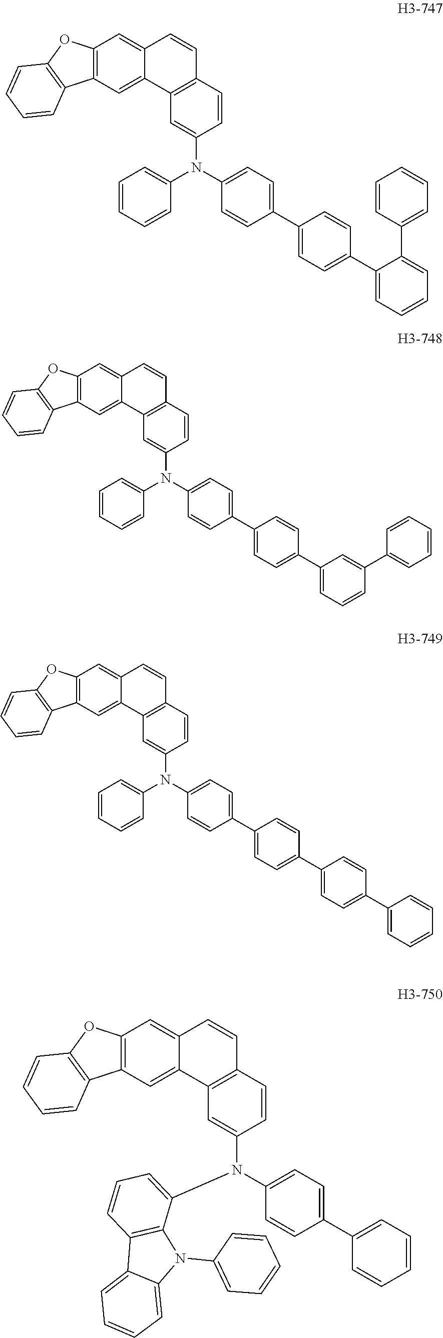 [SMILES: c1ccc(-c2ccccc2-c2ccc(-c3ccc(N(c4ccccc4)c4ccc5ccc6cc7oc8ccccc8c7cc6c5c4)cc3)cc2)cc1 ; c1ccc(-c2cccc(-c3ccc(-c4ccc(N(c5ccccc5)c5ccc6ccc7cc8oc9ccccc9c8cc7c6c5)cc4)cc3)c2)cc1 ; c1ccc(-c2ccc(-c3ccc(-c4ccc(N(c5ccccc5)c5ccc6ccc7cc8oc9ccccc9c8cc7c6c5)cc4)cc3)cc2)cc1 ; c1ccc(-c2ccc(N(c3ccc4ccc5cc6oc7ccccc7c6cc5c4c3)c3cccc4c5ccccc5n(-c5ccccc5)c34)cc2)cc1]

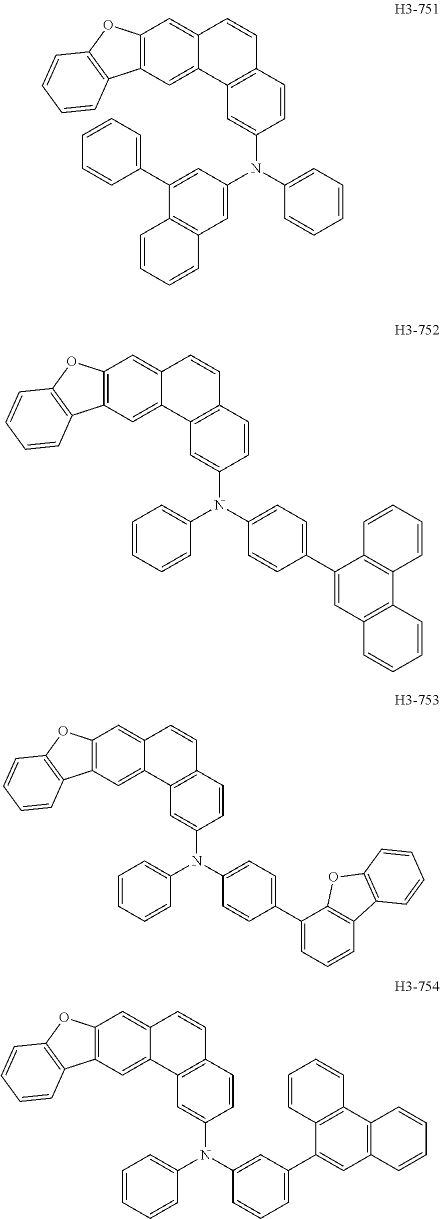 [SMILES: c1ccc(-c2cc(N(c3ccccc3)c3ccc4ccc5cc6oc7ccccc7c6cc5c4c3)cc3ccccc23)cc1 ; c1ccc(N(c2ccc(-c3cc4ccccc4c4ccccc34)cc2)c2ccc3ccc4cc5oc6ccccc6c5cc4c3c2)cc1 ; c1ccc(N(c2ccc(-c3cccc4c3oc3ccccc34)cc2)c2ccc3ccc4cc5oc6ccccc6c5cc4c3c2)cc1 ; c1ccc(N(c2cccc(-c3cc4ccccc4c4ccccc34)c2)c2ccc3ccc4cc5oc6ccccc6c5cc4c3c2)cc1]

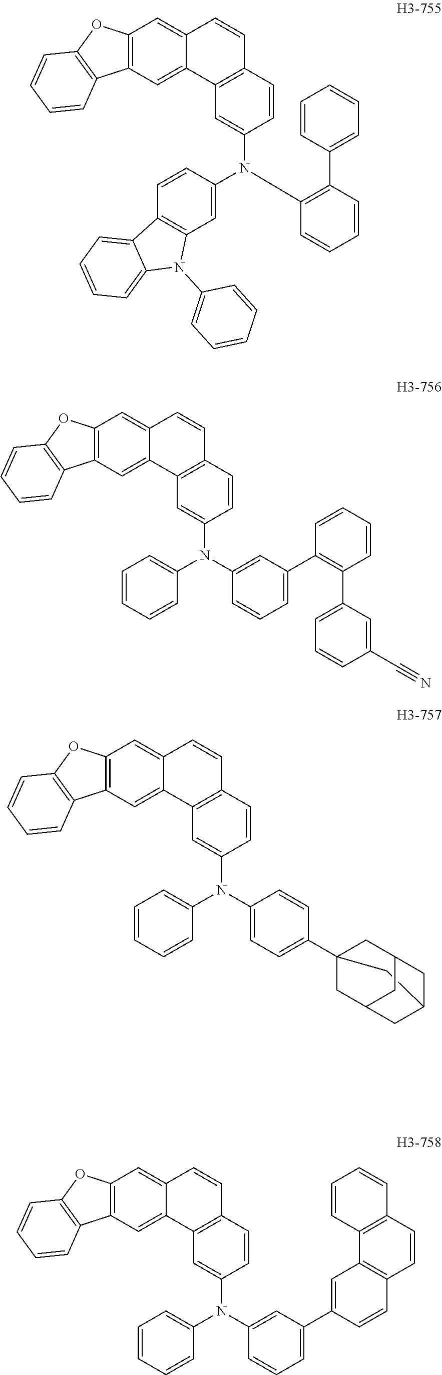 [SMILES: c1ccc(-c2ccccc2N(c2ccc3ccc4cc5oc6ccccc6c5cc4c3c2)c2ccc3c4ccccc4n(-c4ccccc4)c3c2)cc1 ; N#Cc1cccc(-c2ccccc2-c2cccc(N(c3ccccc3)c3ccc4ccc5cc6oc7ccccc7c6cc5c4c3)c2)c1 ; c1ccc(N(c2ccc(C34CC5CC(CC(C5)C3)C4)cc2)c2ccc3ccc4cc5oc6ccccc6c5cc4c3c2)cc1 ; c1ccc(N(c2cccc(-c3ccc4ccc5ccccc5c4c3)c2)c2ccc3ccc4cc5oc6ccccc6c5cc4c3c2)cc1]

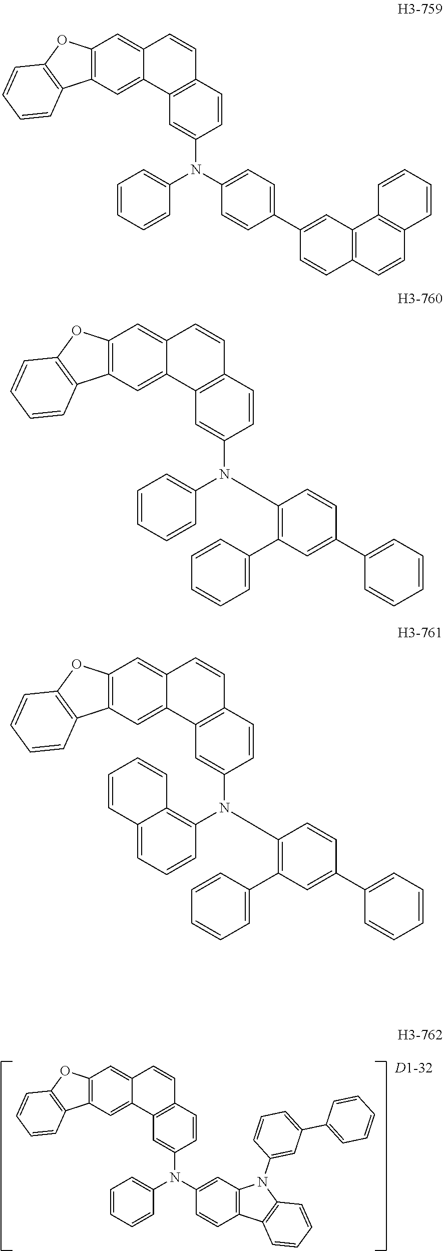

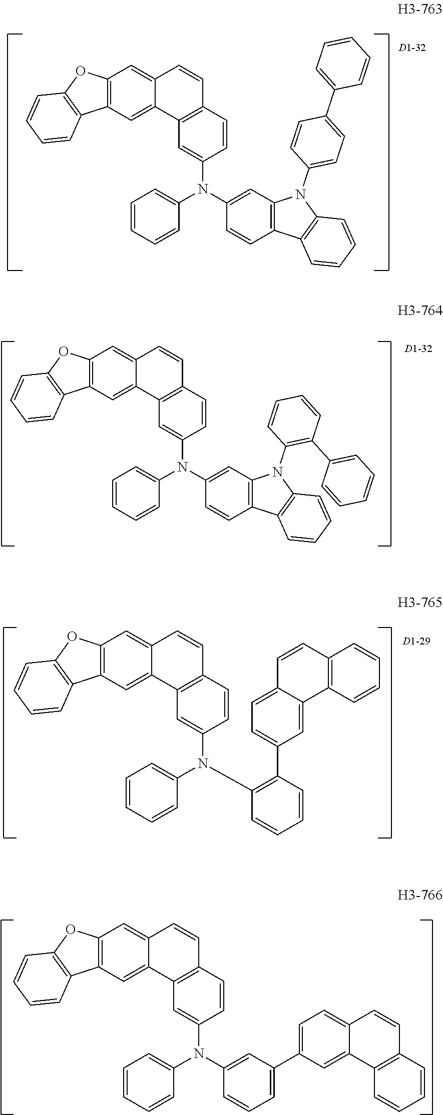

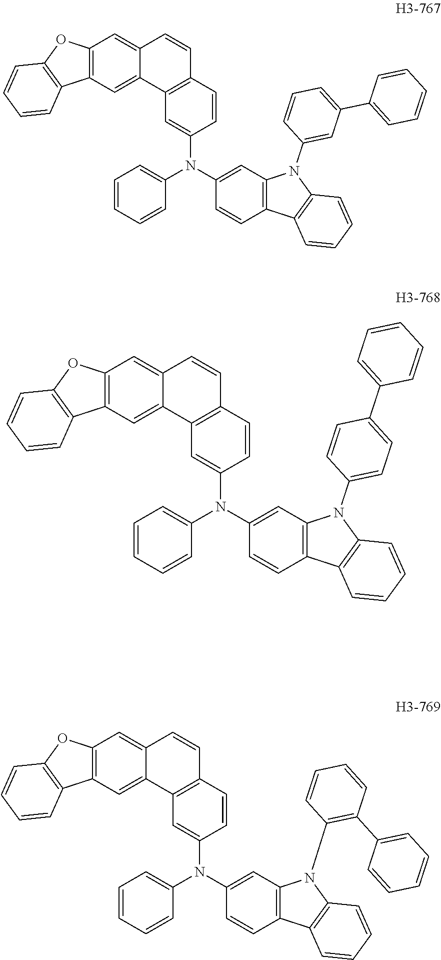 [SMILES: c1ccc(-c2cccc(-n3c4ccccc4c4ccc(N(c5ccccc5)c5ccc6ccc7cc8oc9ccccc9c8cc7c6c5)cc43)c2)cc1 ; c1ccc(-c2ccc(-n3c4ccccc4c4ccc(N(c5ccccc5)c5ccc6ccc7cc8oc9ccccc9c8cc7c6c5)cc43)cc2)cc1 ; c1ccc(-c2ccccc2-n2c3ccccc3c3ccc(N(c4ccccc4)c4ccc5ccc6cc7oc8ccccc8c7cc6c5c4)cc32)cc1]

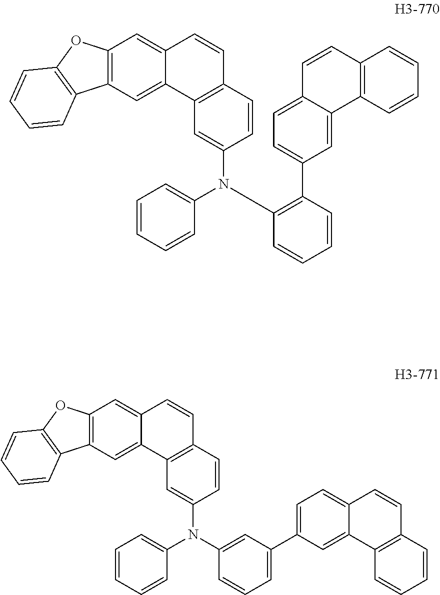 [SMILES: c1ccc(N(c2ccc3ccc4cc5oc6ccccc6c5cc4c3c2)c2ccccc2-c2ccc3ccc4ccccc4c3c2)cc1 ; c1ccc(N(c2cccc(-c3ccc4ccc5ccccc5c4c3)c2)c2ccc3ccc4cc5oc6ccccc6c5cc4c3c2)cc1]

The combination of at least one of compounds H1-1 to H1-315 and at least one of compounds H2-1 to H2-276 may be used in an organic electroluminescent device.

In addition, the combination of at least one of compounds H1-1 to H1-315, at least one of compounds H2-1 to H2-276, and at least one of compounds H3-1 to H3-771 may be used in an organic electroluminescent device.

The compound represented by formula 1 according to the present disclosure may be produced by a synthetic method known to one skilled in the art, and in particular by using the synthetic methods disclosed in a number of patent literatures, for example, by referring to the methods disclosed in Korean Patent Application Laying-Open No. 2018-0099487 (published on Sep. 5, 2018), Korean Patent Application Laying-Open No. 2021-0098316 (published on Aug. 10, 2021), Korean Patent Application Laying-Open No. 2022-0051794 (published on Apr. 26, 2022), and Korean Patent Application Laying-Open No. 2021-0109436 published on Sep. 6, 2021), etc., but is not limited thereto.

The compound represented by formula 2 according to the present disclosure may be produced by a synthetic method known to one skilled in the art, and in particular by using the synthetic methods disclosed in a number of patent literatures, for example, by referring to the methods disclosed in Korean Patent Application Laying-Open No. 2022-0051794 (published on Apr. 26, 2022), Korean Patent Application Laying-Open No. 2021-0124018 (published on Oct. 14, 2021), and Korean Patent Application Laying-Open No. 2021-0109436 (published on Sep. 6, 2021), etc., but is not limited thereto.

The compound represented by formula 3 according to the present disclosure may be produced by referring to the following reaction scheme 1, but is not limited thereto, and may be produced by a synthetic method known to one skilled in the art.

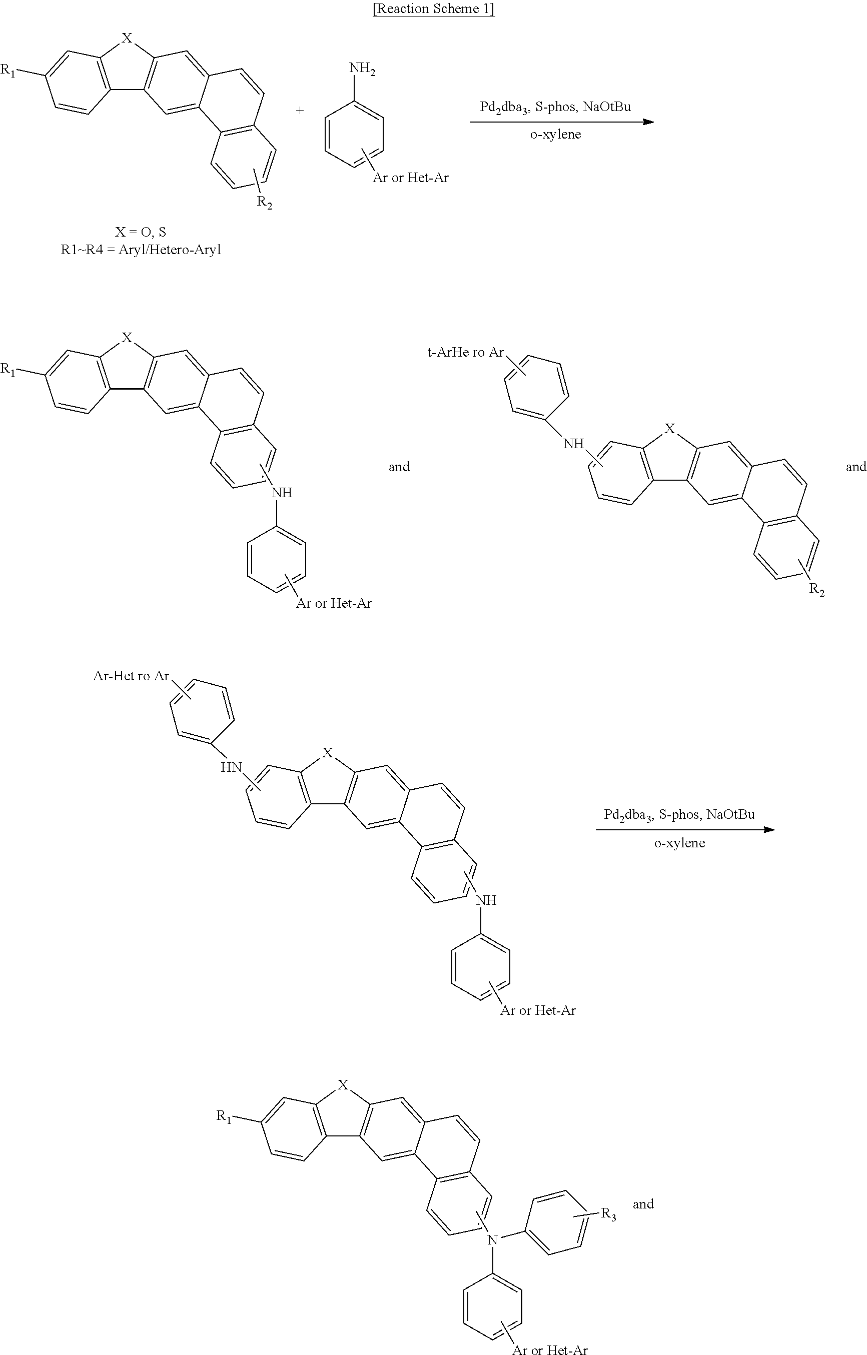

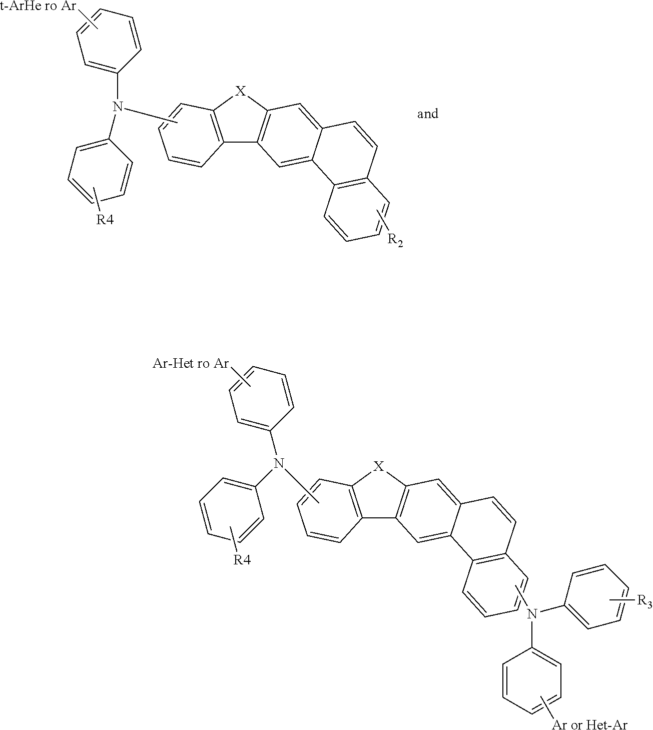

Although illustrative synthesis examples of the compounds represented by formulas 1 to 3 of the present disclosure are described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN1 substitution reaction, an SN2 substitution reaction, and a Phosphine-mediated reductive cyclization reaction, etc., and the reactions above proceed even when substituents which are defined in formulas 1 to 3 above, but are not specified in the specific synthesis examples, are bonded.

In addition, the deuterated compounds of formulas 1 to 3 may be prepared in a similar manner by using deuterated precursor materials, or more generally may be prepared by treating the non-deuterated compound with a deuterated solvent or D6-benzene in the presence of an H/D exchange catalyst such as a Lewis acid, e.g., aluminum trichloride or ethyl aluminum chloride. In addition, the degree of deuteration can be controlled by changing the reaction conditions such as the reaction temperature. For example, the number of deuterium in formulas 1 to 3 can be controlled by adjusting the reaction temperature and time, the equivalent of the acid, etc.

The present disclosure provides an organic electroluminescent device comprising the organic electroluminescent compound according to the present disclosure of a specific formula 1′.

The present disclosure provides an organic electroluminescent device comprising an anode, a cathode, and at least one light-emitting layer between the anode and cathode in which the at least one light-emitting layer comprises the plurality of host materials according to the present disclosure. The first host material and the second host material or the first host material to the third host material according to the present disclosure may be comprised in one light-emitting layer, or may be respectively comprised in different light-emitting layers. In the plurality of host materials of the present disclosure, for example, the ratio of the compound represented by formula 1 and the compound represented by formula 2 is about 1:99 to about 99:1, preferably about 10:90 to about 90:10, more preferably about 30:70 to about 70:30. In addition, the compound represented by formula 1 and the compound represented by formula 2, or the compound represented by formulas 1 to 3 in a desired ratio may be combined by mixing them in a shaker, by dissolving them in a glass tube by heat, or by dissolving them in a solvent, etc.

Herein, the first host material among the plurality of host materials of the present disclosure may be about 5 to about 90% by weight, preferably about 10 to about 90% by weight, more preferably about 10 to about 80% by weight, even more preferably about 15 to about 70% by weight, further more preferably about 30 to about 70% by weight, and further more preferably about 30 to about 60% by weight. Among the plurality of host materials of the present disclosure, the second host material may be about 5 to about 90% by weight, preferably about 10 to about 90% by weight, more preferably about 10 to about 80% by weight, even more preferably about 15 to about 70% by weight, further more preferably about 30 to about 70% by weight, and further more preferably about 30 to about 60% by weight. Among the plurality of host materials of the present disclosure, the third host material may be about 5 to about 90% by weight, preferably about 10 to about 90% by weight, more preferably about 10 to about 80% by weight, even more preferably about 15 to about 70% by weight, further more preferably about 30 to about 70% by weight, and further more preferably about 30 to about 60% by weight. For example, the plurality of host materials include about 5 to about 70% by weight of the first host material, about 5 to about 70% by weight of the second host material, and about 10 to about 90% by weight of the third host material.

According to one embodiment of the present disclosure, the doping concentration of the dopant compound with respect to the host compound in the light-emitting layer may be less than 20 wt %. The dopant comprised in the organic electroluminescent device of the present disclosure may be at least one phosphorescent or fluorescent dopant, and is preferably a phosphorescent dopant. The phosphorescent dopant material applied to the organic electroluminescent device of the present disclosure is not particularly limited, but may be preferably selected from the group consisting of the metallated complex compounds of iridium (Ir), osmium (Os), copper (Cu), and platinum (Pt), more preferably selected from the group consisting of ortho-metallated complex compounds of iridium (Ir), osmium (Os), copper (Cu), and platinum (Pt), and even more preferably ortho-metallated iridium complex compounds.

The dopant comprised in the organic electroluminescent device of the present disclosure may be a compound represented by the following formula 101, but is not limited thereto.

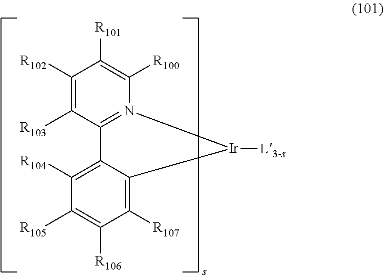

In formula 101,
L′ is selected from the following structures 1 to 3:

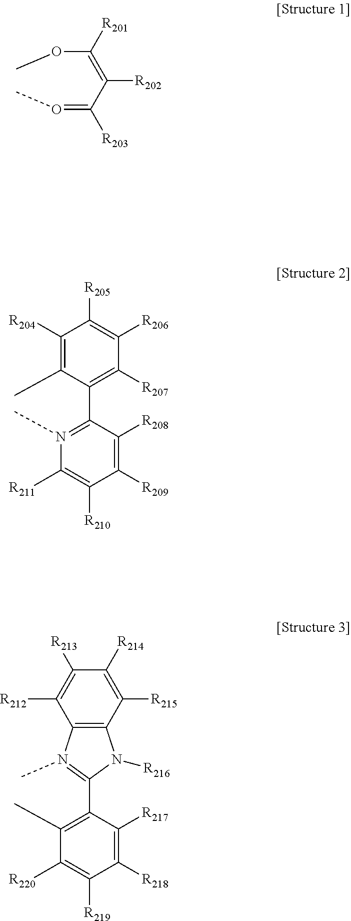

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent to form a ring(s), e.g., a substituted or unsubstituted, quinoline, benzofuropyridine, benzothienopyridine, indenopyridine, benzofuroquinoline, benzothienoquinoline, or indenoquinoline, together with pyridine;
R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent to form a substituted or unsubstituted ring(s), e.g., a substituted or unsubstituted, naphthalene, fluorene, dibenzothiophene, dibenzofuran, indenopyridine, benzofuropyridine, or benzothienopyridine, together with benzene;
R₂₀₁ to R₂₂₀, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent to form a substituted or unsubstituted ring(s); and
s represents an integer of 1 to 3.

The specific examples of the dopant compound are as follows, but are not limited thereto.

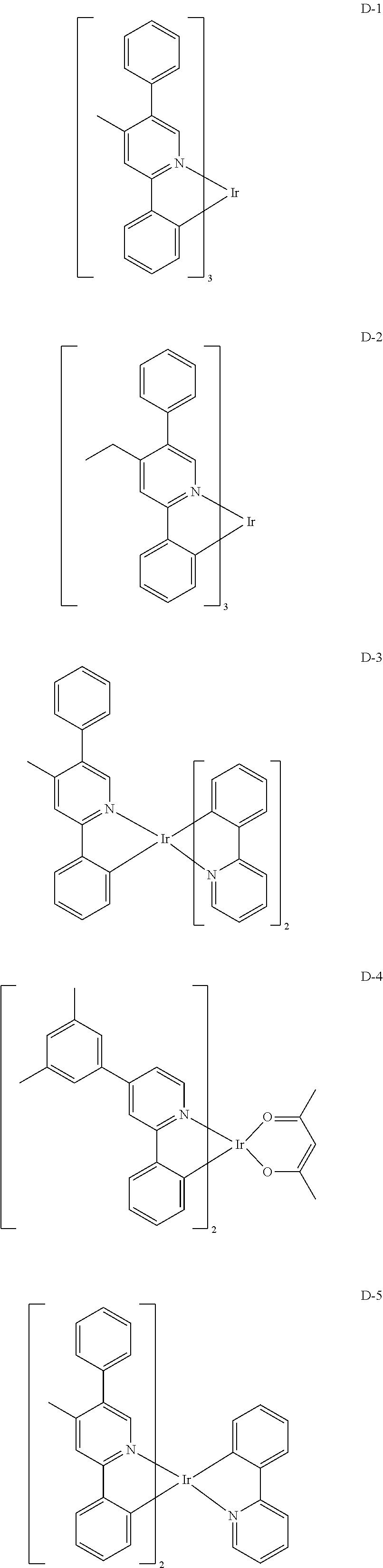

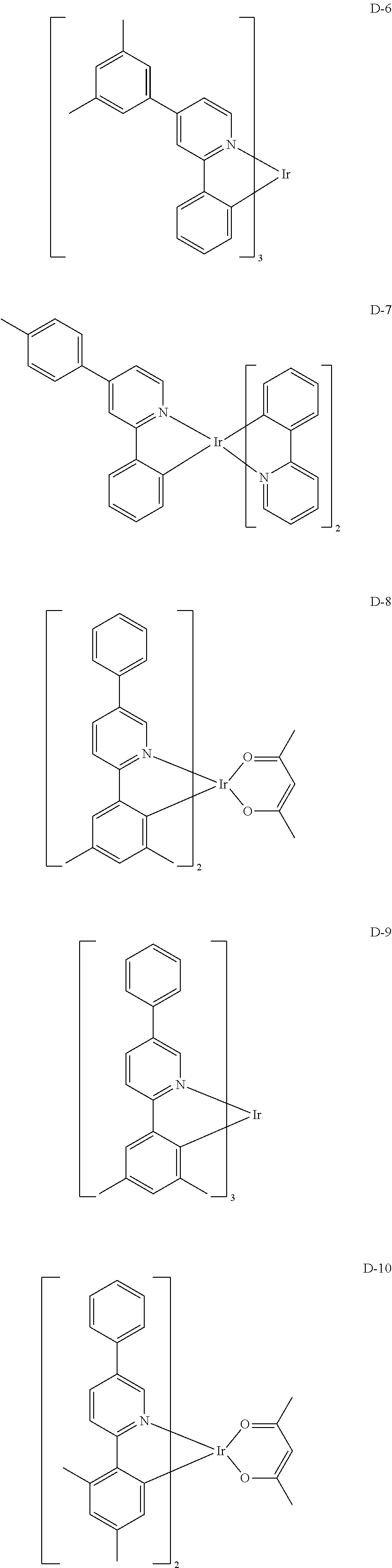

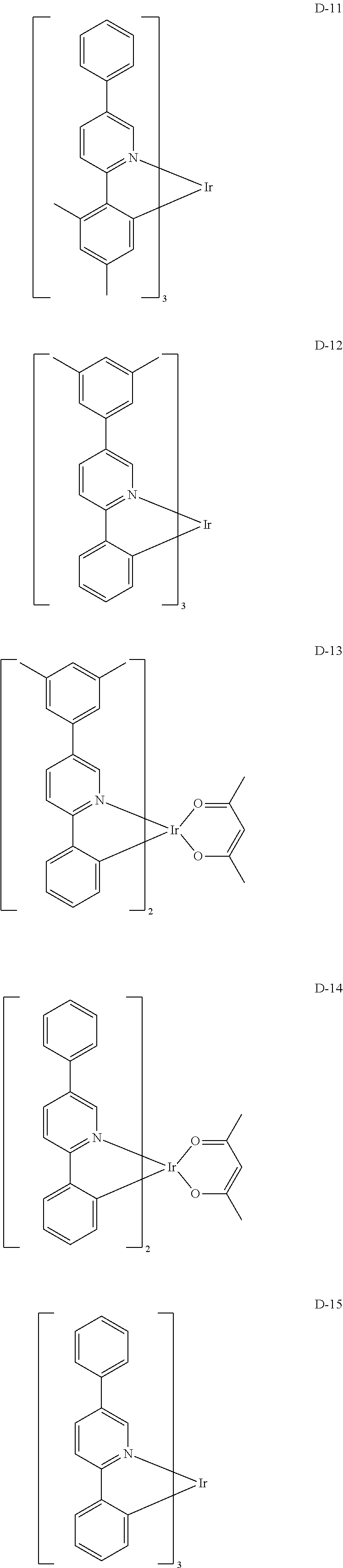

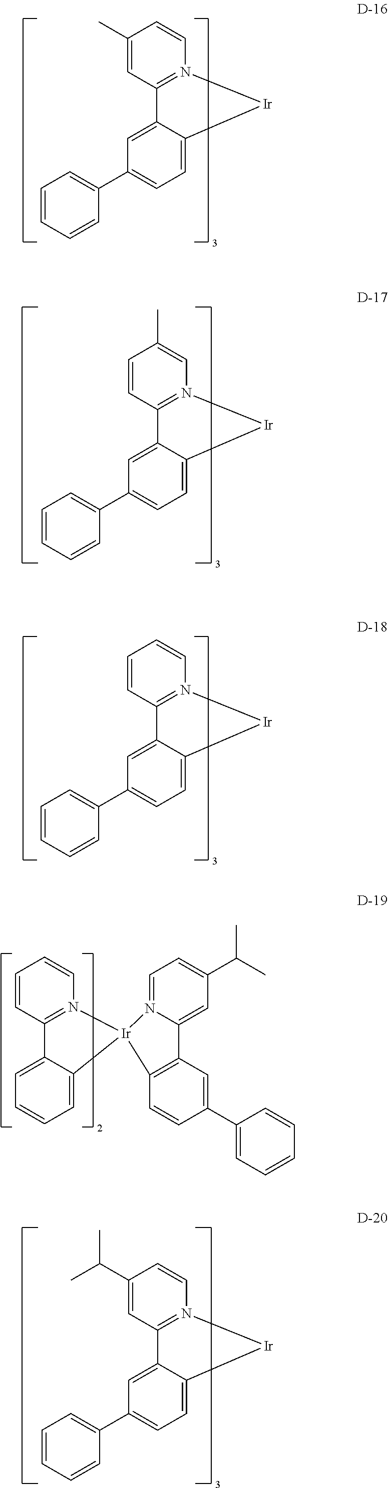

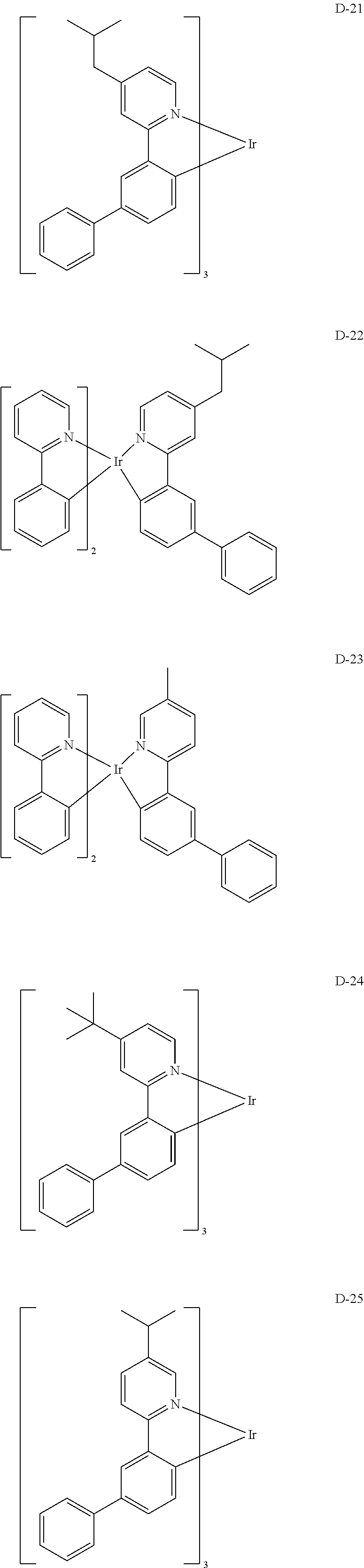

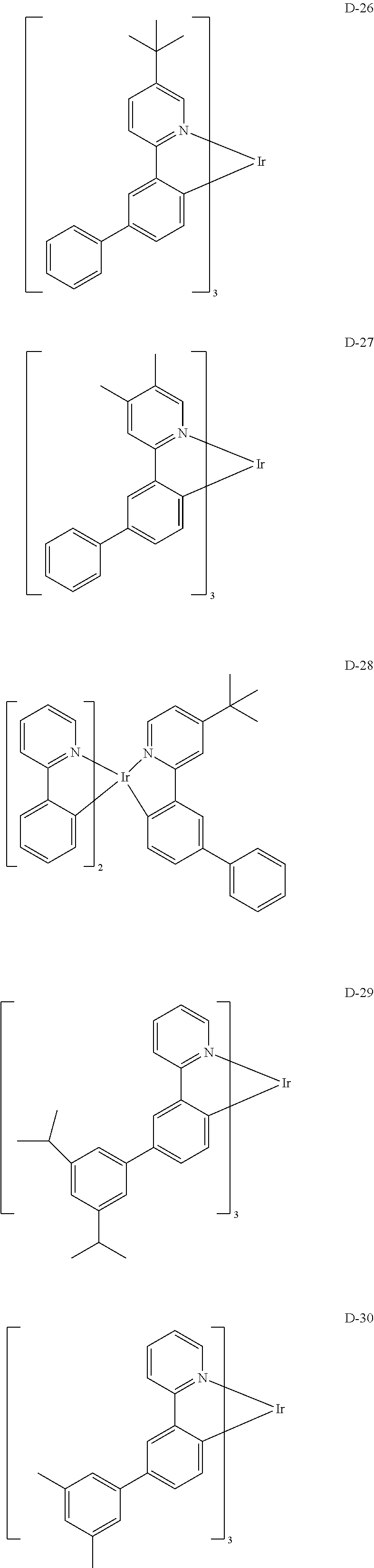

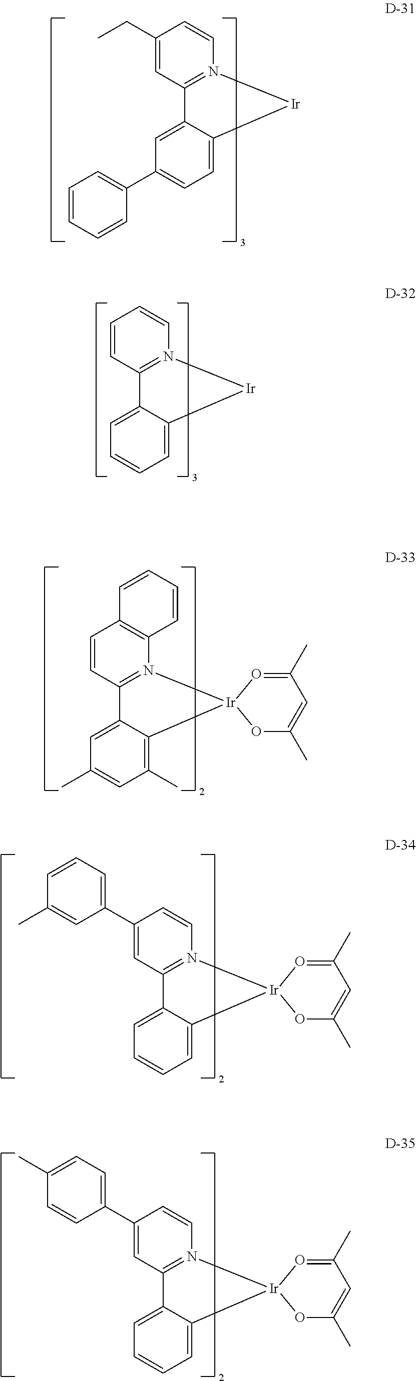

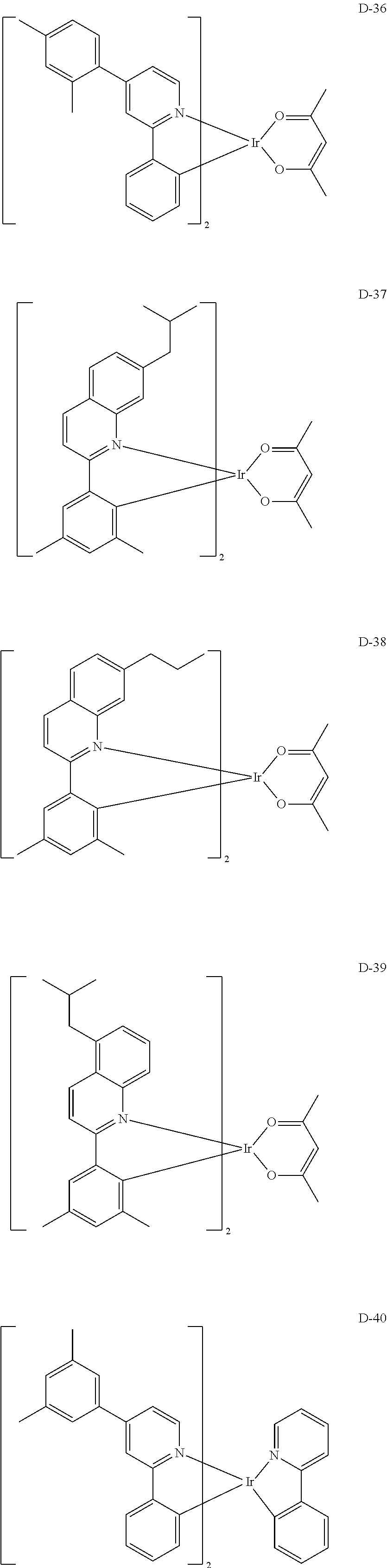

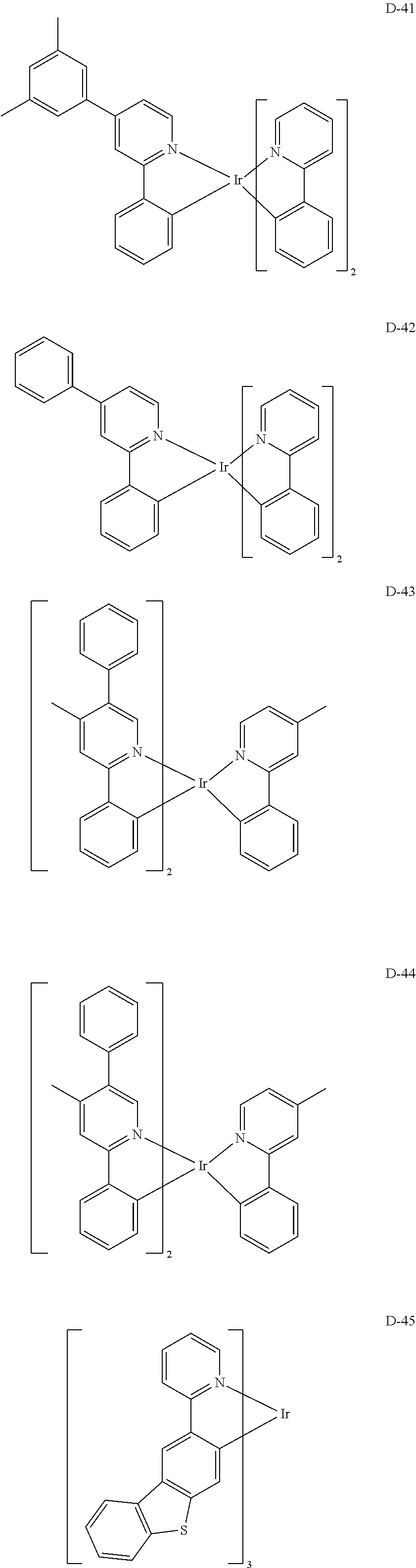

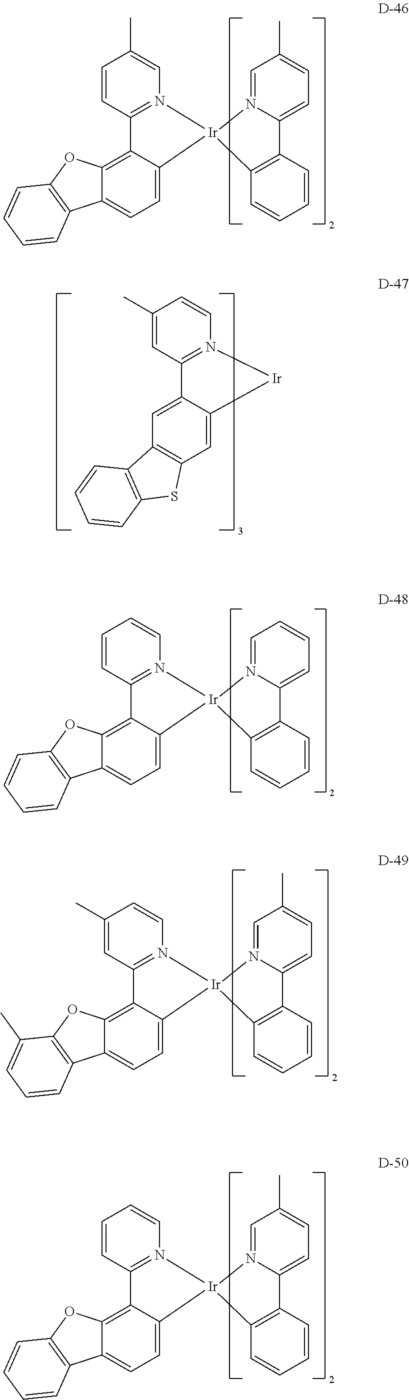

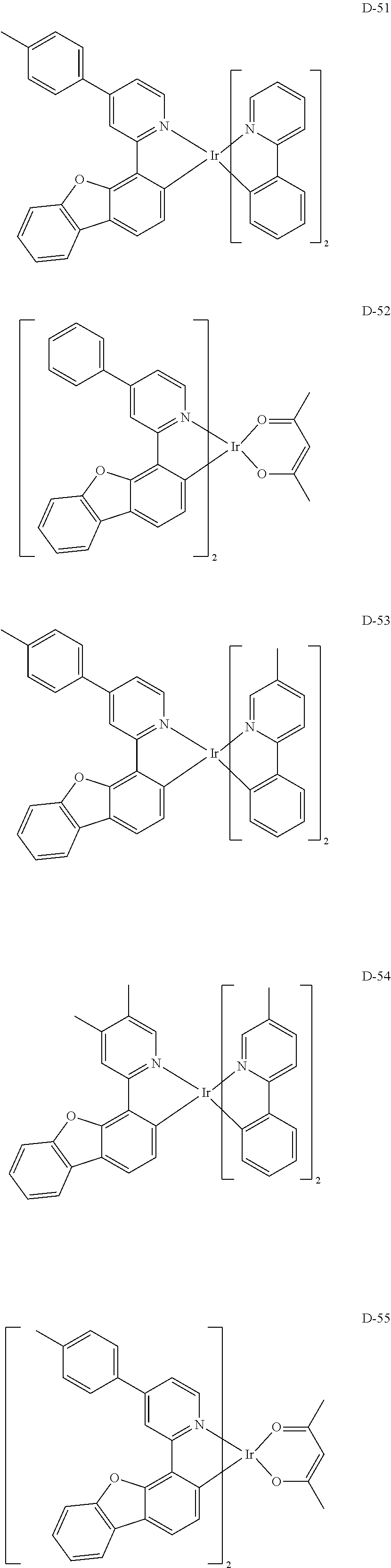

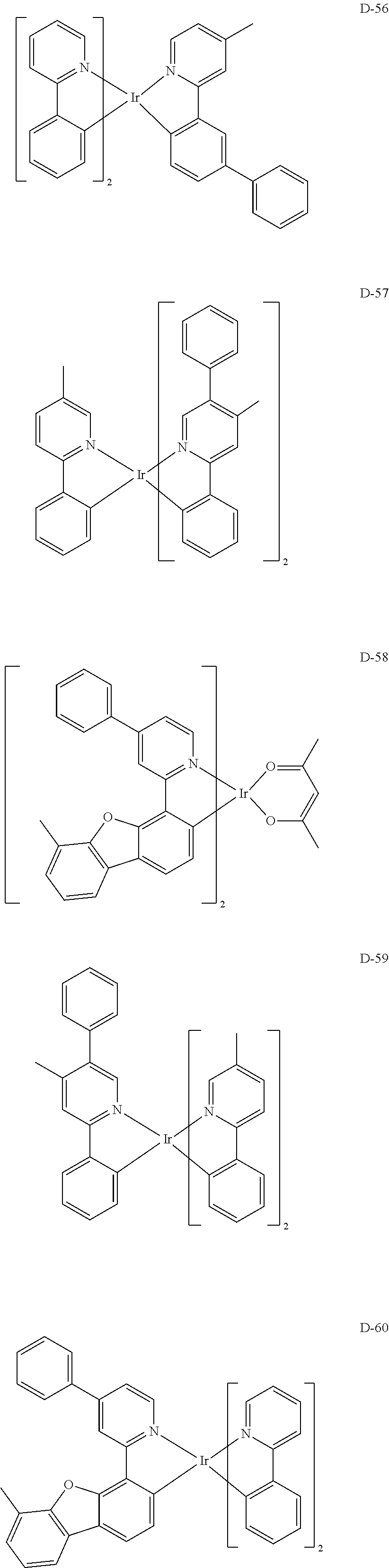

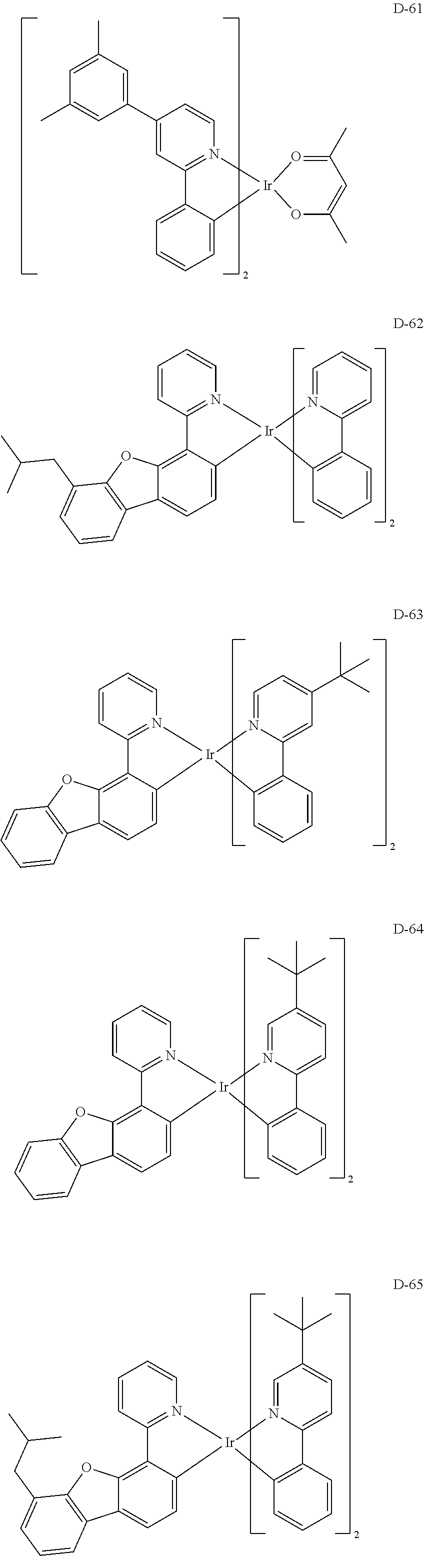

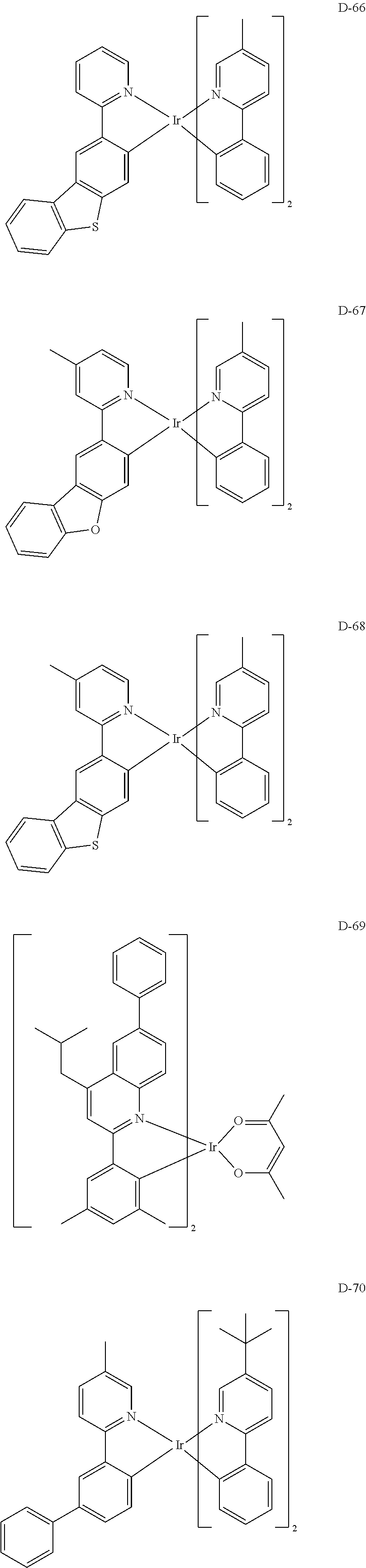

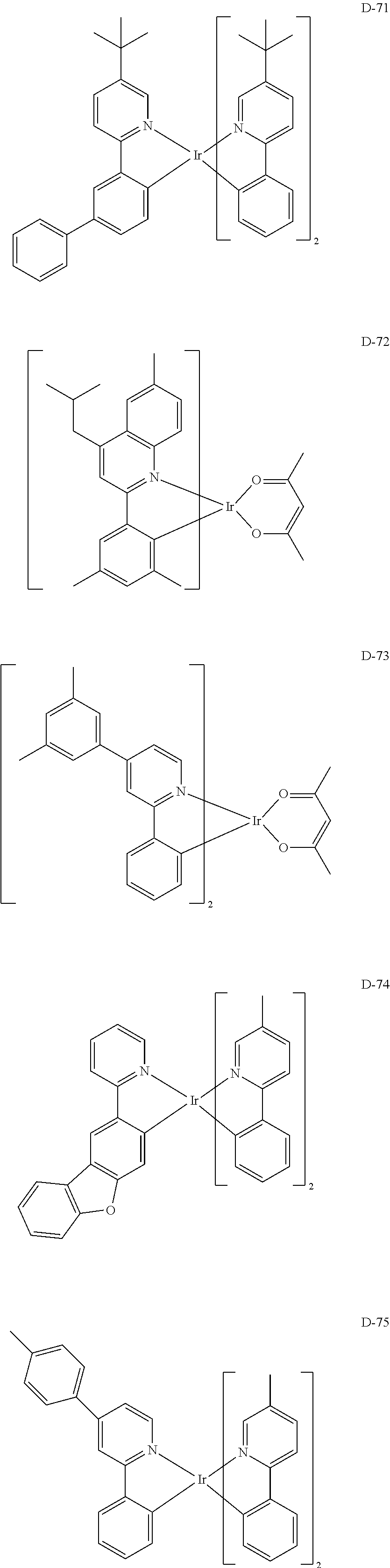

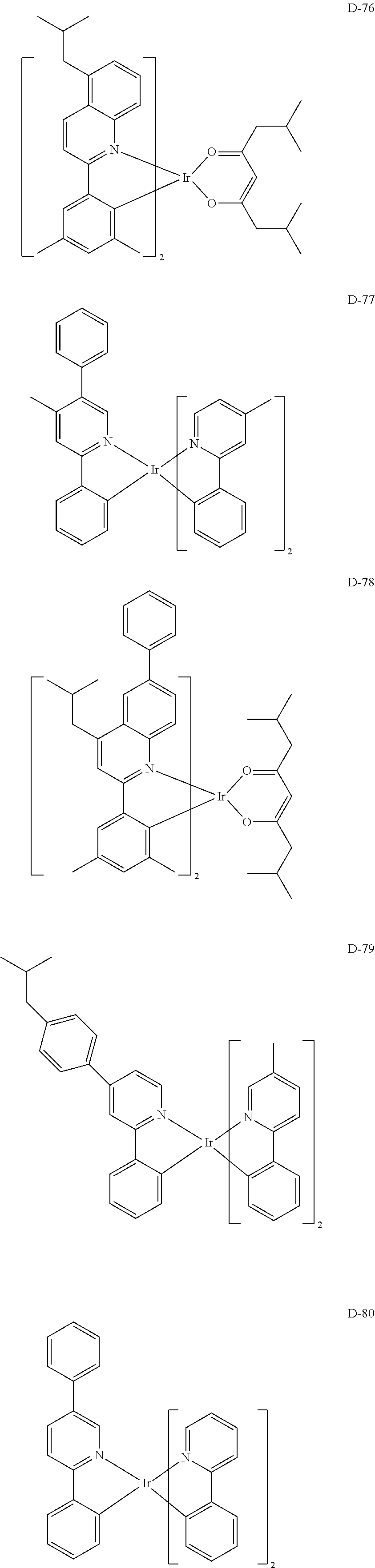

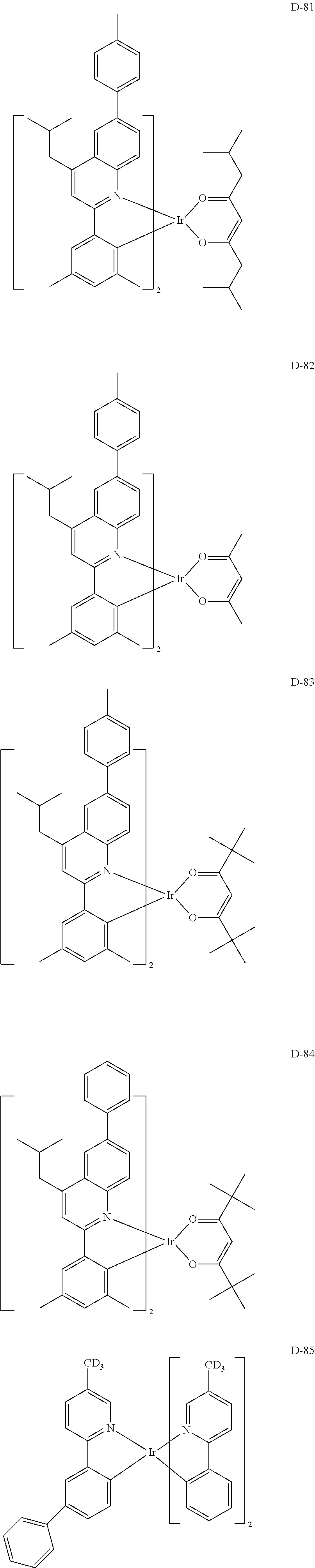

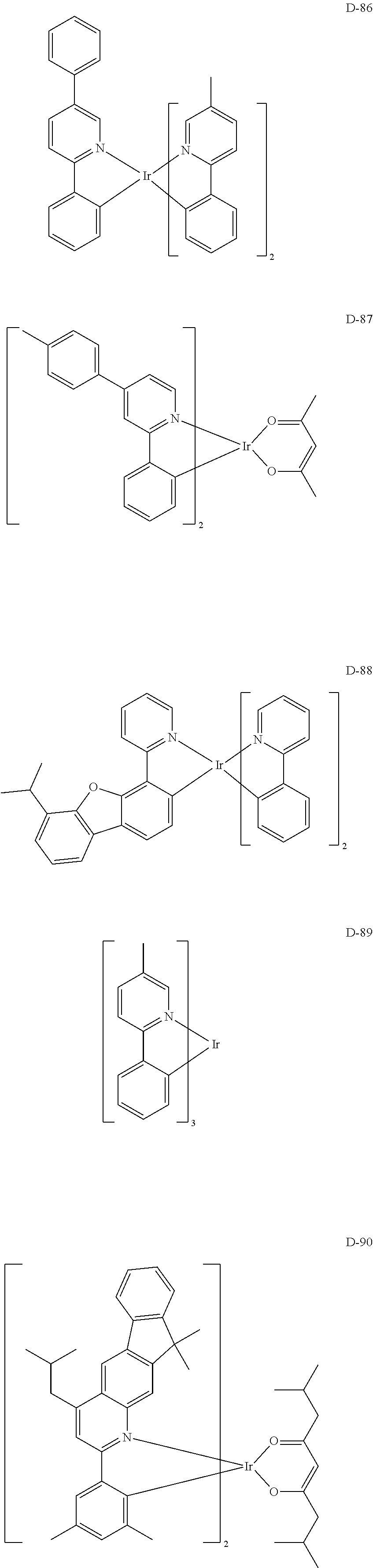

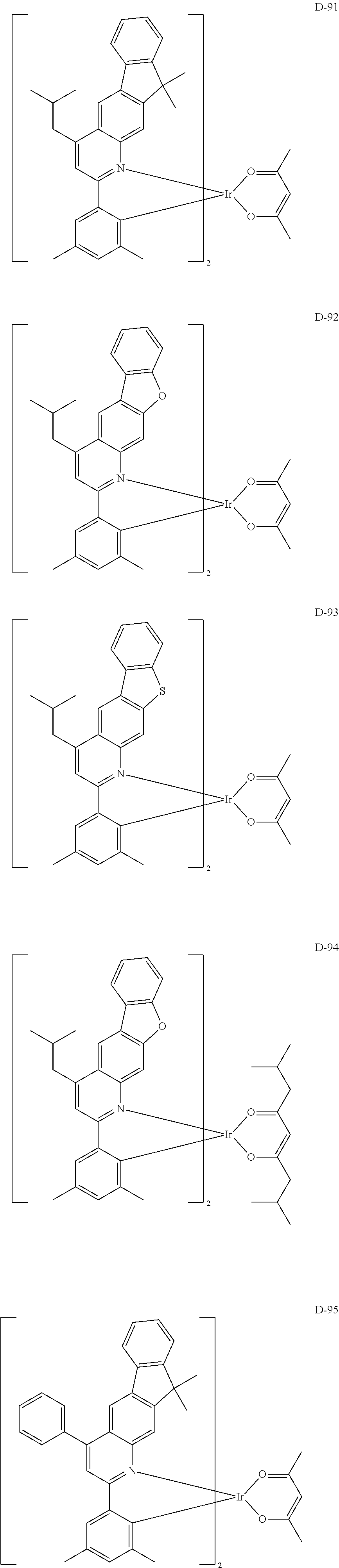

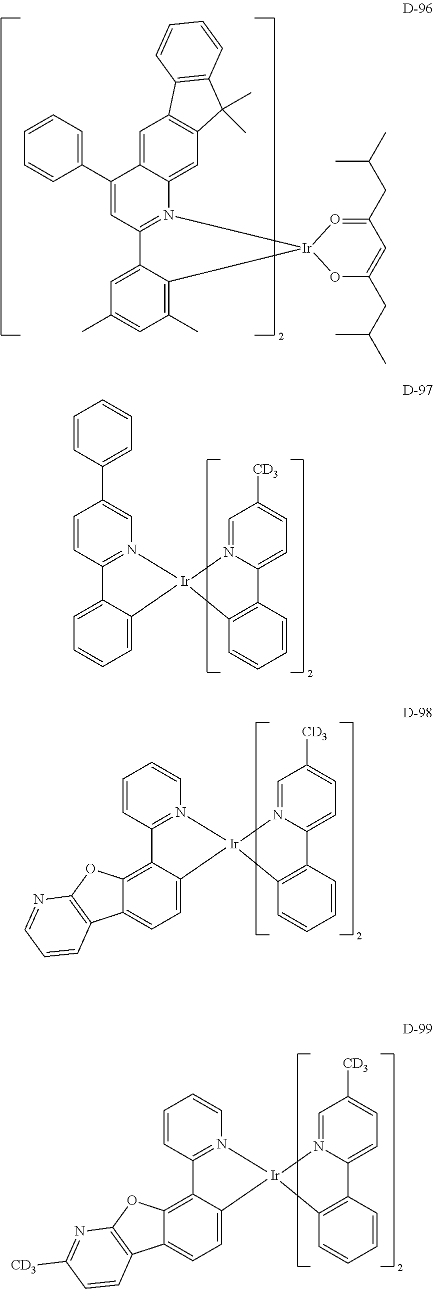

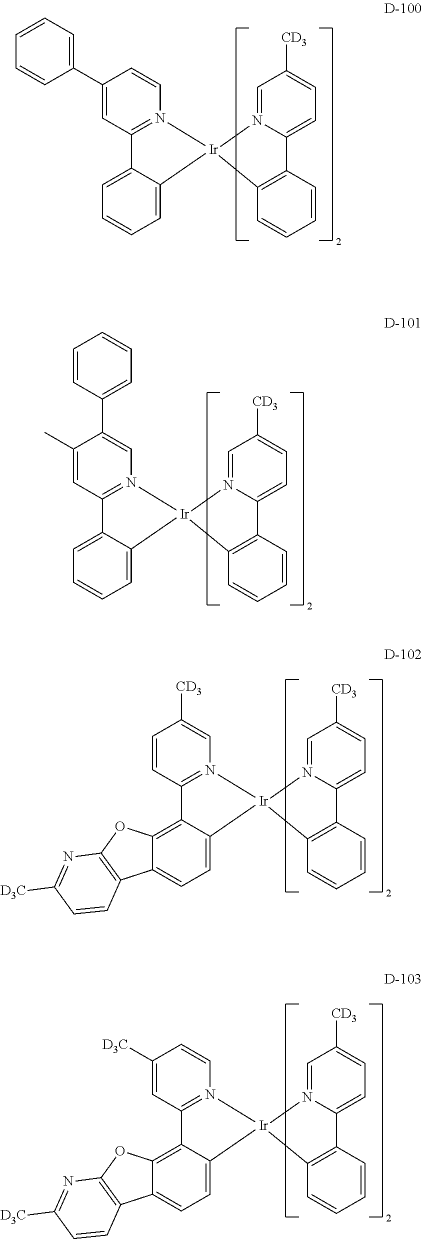

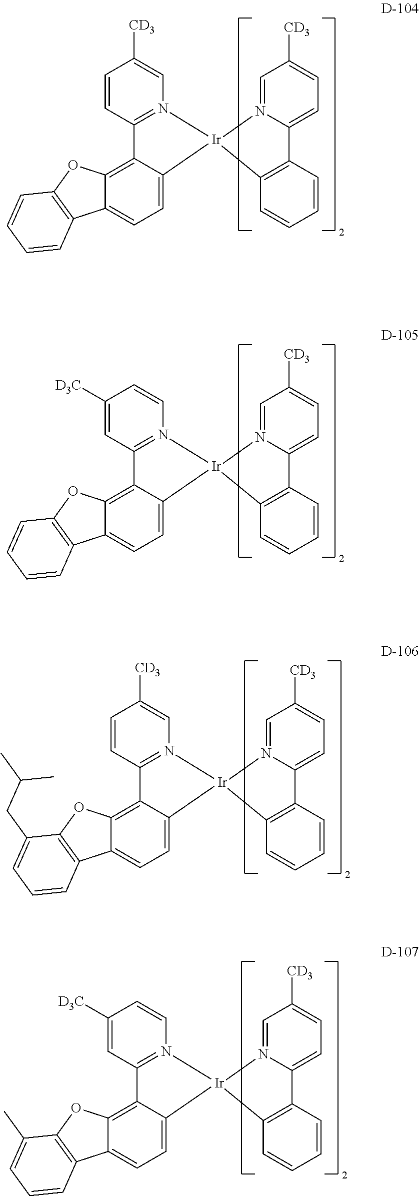

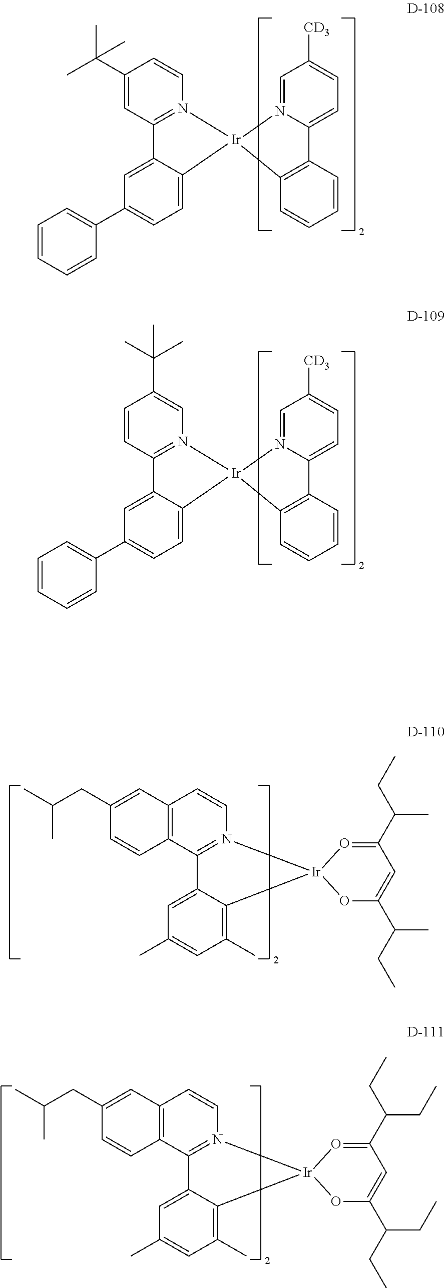

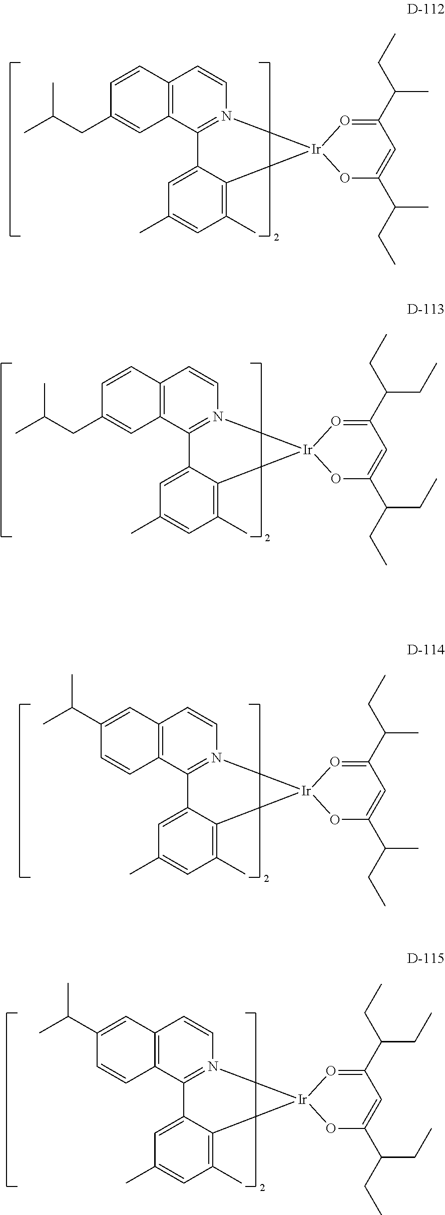

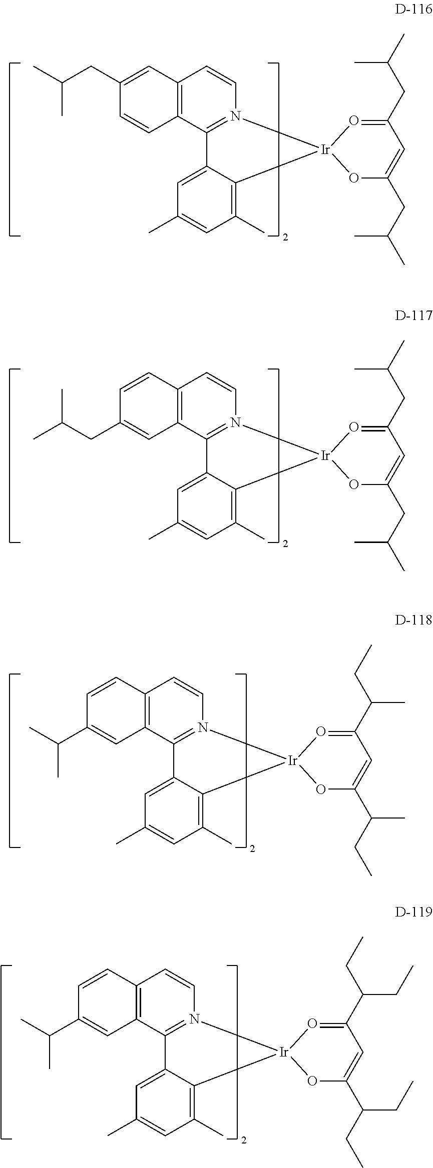

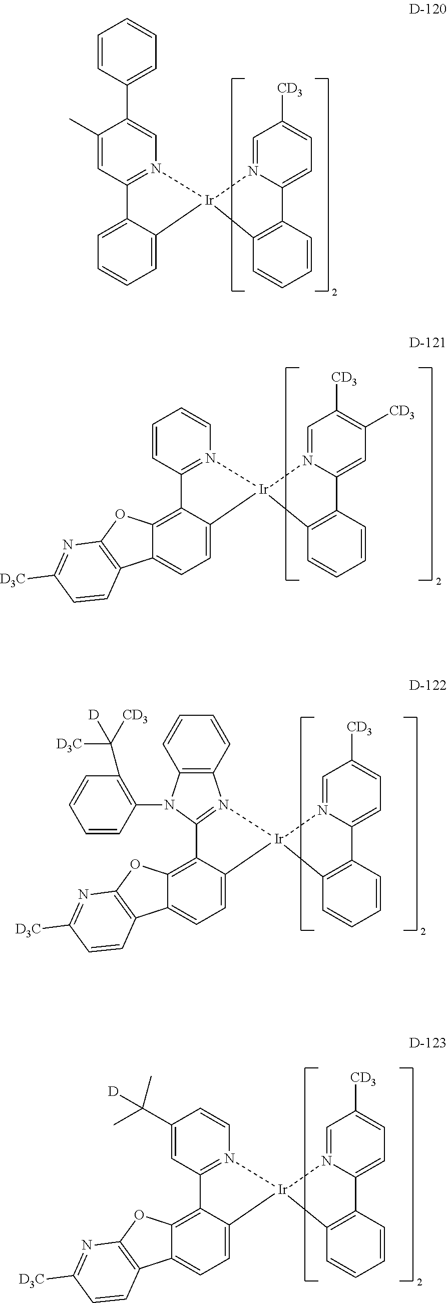

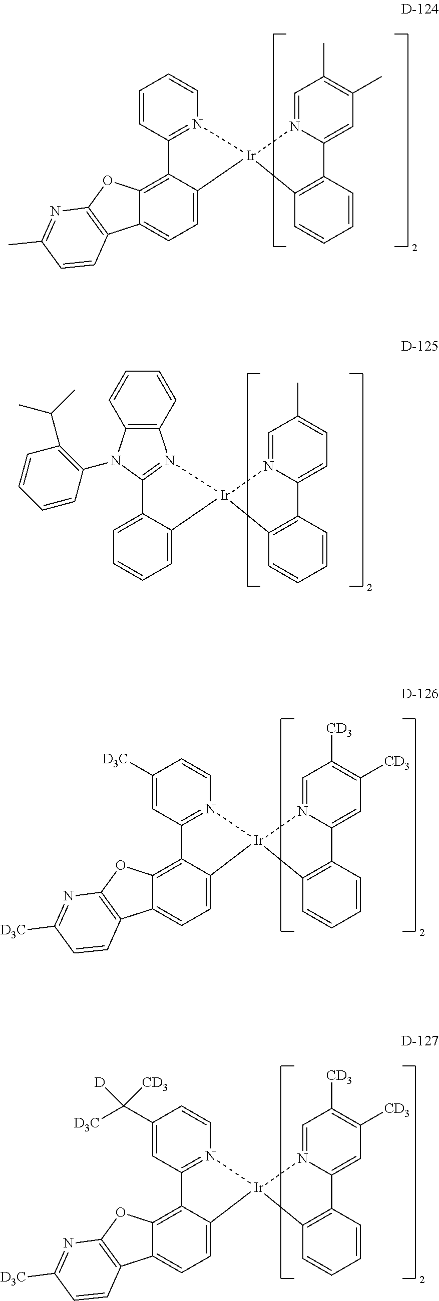

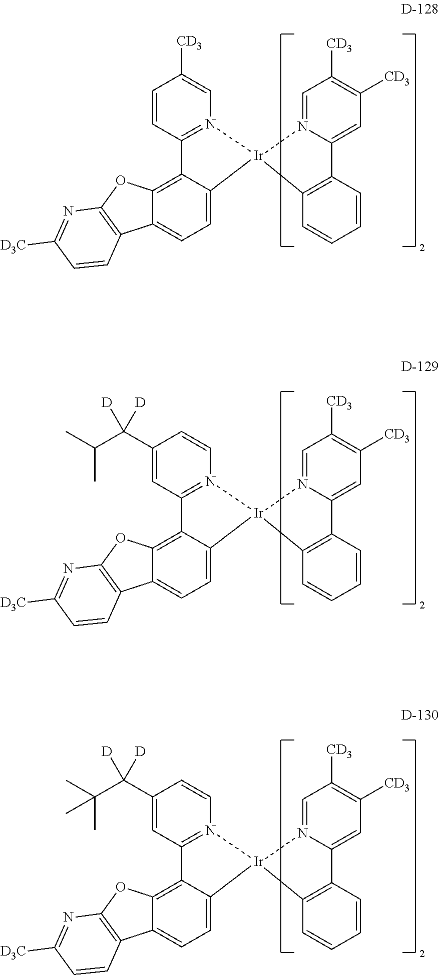

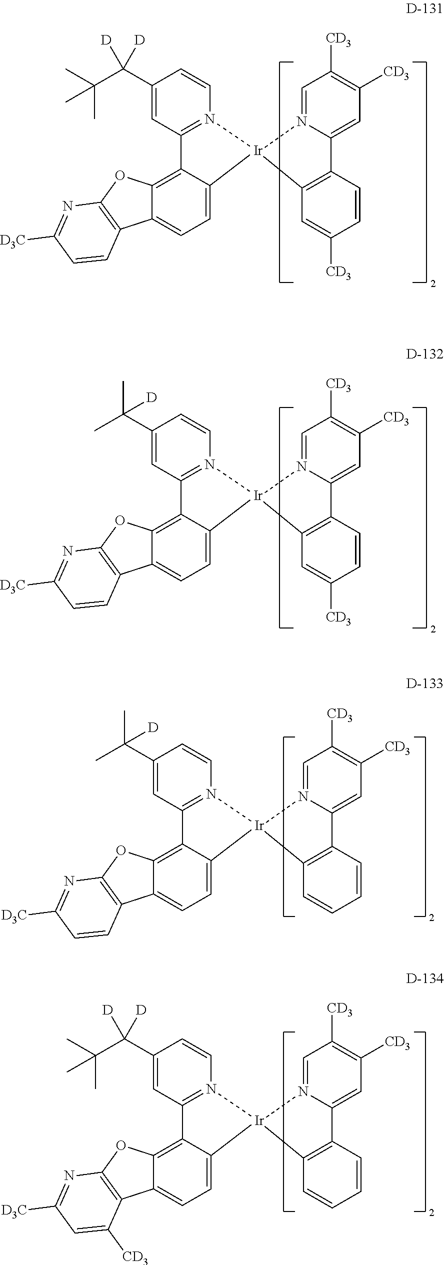

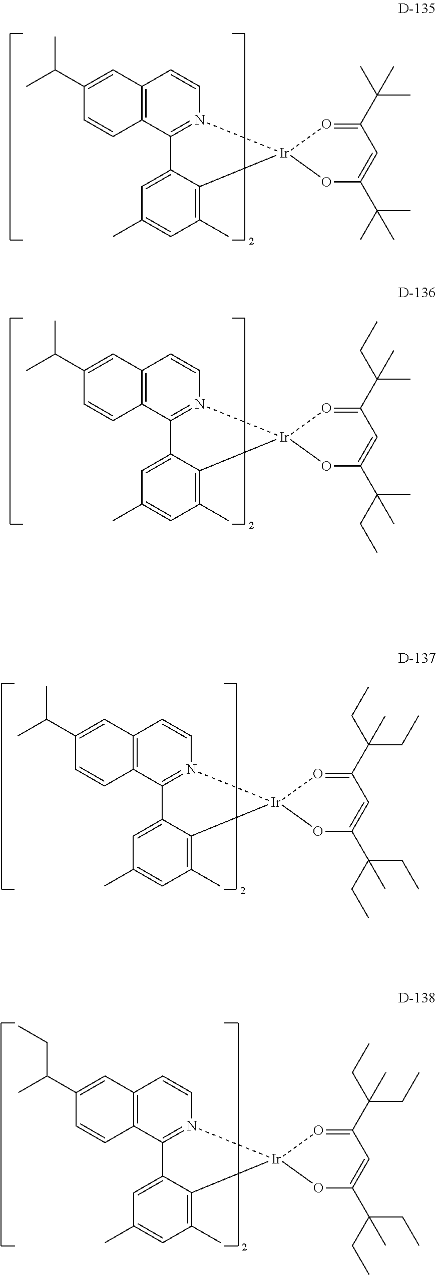

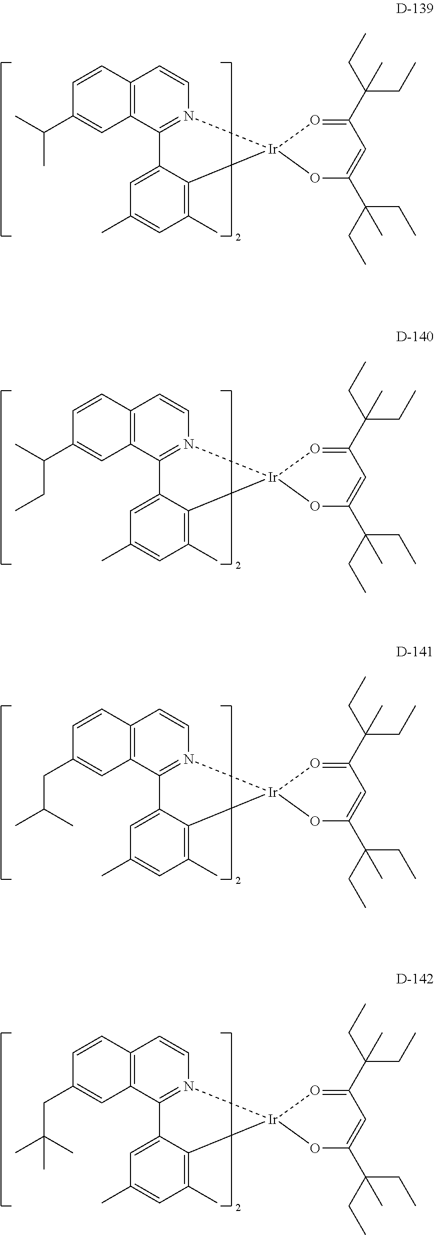

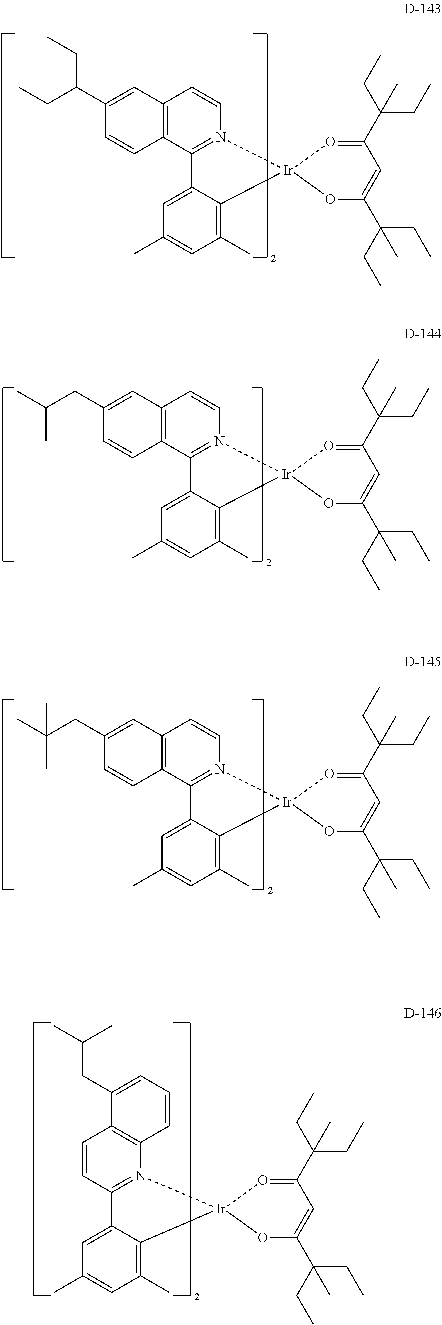

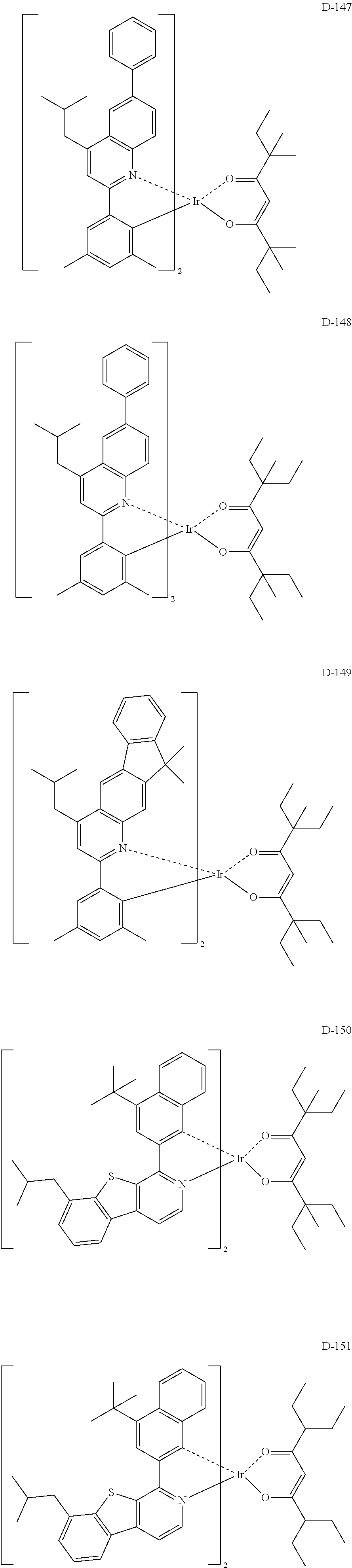

An organic electroluminescent device according to the present disclosure has an anode, a cathode, and at least one organic layer between the anode and the cathode. The organic layer comprises a light-emitting layer and may further comprise at least one layer selected from the group consisting of a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Each of the layers may be further configured as a plurality of layers.

The anode and the cathode may be respectively formed with a transparent conductive material, or a transflective or reflective conductive material. The organic electroluminescent device may be a top emission type, a bottom emission type, or a both-sides emission type, depending on the materials forming the anode and the cathode. In addition, the hole injection layer may be further doped with a p-dopant, and the electron injection layer may be further doped with an n-dopant.

The organic layer may further comprise at least one compound selected from the group consisting of arylamine-based compounds and styrylarylamine-based compounds. Further, the organic layer may further comprise at least one metal selected from the group consisting of metals of Group 1, metals of Group 2, transition metals of the 4th period, transition metals of the 5th period, lanthanides, and organic metals of the d-transition elements of the Periodic Table, or at least one complex compound comprising the metal.

In addition, the organic electroluminescent device of the present disclosure may emit white light by further comprising at least one light-emitting layer, which comprises a blue, a red, or a green electroluminescent compound known in the field, besides the compound of the present disclosure. If necessary, it may further comprise a yellow or an orange light-emitting layer.

In the organic electroluminescent device of the present disclosure, preferably, at least one layer selected from the group consisting of a chalcogenide layer, a metal halide layer, and a metal oxide layer (hereinafter, “a surface layer”) may be placed on an inner surface(s) of one or both electrode(s). Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, the chalcogenide includes SiOₓ(1≤X≤2), AlOₓ(1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. The hole transport layer or the electron blocking layer may also be multi-layers.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer may also be multi-layers, wherein each of the multi-layers may use a plurality of compounds.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or hole transport, or for preventing the overflow of electrons. When the light-emitting auxiliary layer is placed between the cathode and the light-emitting layer, it can be used for promoting the electron injection and/or electron transport, or for preventing the overflow of holes. Also, the hole auxiliary layer may be placed between the hole transport layer (or hole injection layer) and the light-emitting layer, and may be effective to promote or block the hole transport rate (or hole injection rate), thereby enabling the charge balance to be controlled. Further, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. When an organic electroluminescent device includes two or more hole transport layers, the hole transport layer, which is further included, may be used as a hole auxiliary layer or an electron blocking layer. The light-emitting auxiliary layer, the hole auxiliary layer or the electron blocking layer may have an effect of improving the efficiency and/or the lifetime of the organic electroluminescent device.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferably, the oxidative dopant includes various Lewis acids and acceptor compounds; and the reductive dopant includes alkali metals, alkali metal compounds, alkaline earth metals, rare-earth metals, and mixtures thereof. The reductive dopant layer may be employed as a charge-generating layer to produce an organic electroluminescent device having two or more light-emitting layers and emitting white light.

The organic electroluminescent material according to the present disclosure may be used as a light-emitting material for a white organic light-emitting device. The white organic light-emitting device has been suggested to have various structures such as a side-by-side structure or a stacking structure depending on the arrangement of R (red), G (green) or YG (yellow green), and B (blue) light-emitting parts, or color conversion material (CCM) method, etc. The organic electroluminescent material according to the present disclosure may also be used in an organic electroluminescent device comprising a quantum dot (QD).

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used. When the first to third host compounds of the present disclosure are used to form a film, a co-evaporation process or a mixture-evaporation process is carried out.

When using a wet film-forming method, a thin film can be formed by dissolving or diffusing materials forming each layer into any suitable solvent such as ethanol, chloroform, tetrahydrofuran, dioxane, etc. The solvent can be any one where the materials forming each layer can be dissolved or diffused, and where there are no problems in film-formation capability.

The organic electroluminescent device according to one embodiment of the present disclosure may be an organic electroluminescent device having a tandem structure. According to one embodiment of a tandem organic electroluminescent device, a single light-emitting unit (light-emitting unit) may be formed in a structure in which two or more light-emitting units are connected by a charge generation layer. The organic electroluminescent device may comprise a plurality of two or more light-emitting units having first and second electrodes opposed to each other on a substrate and a light-emitting layer that is stacked between the first and second electrodes and emits light in a specific wavelength range, for example, it may comprise a plurality of three or more light-emitting units. It may comprise a plurality of light-emitting units, and each light-emitting unit may comprise a hole transport zone, a light-emitting layer, and an electron transport band, and the hole transport zone may comprise a hole injection layer and a hole transport layer. The transfer band may include an electron transfer layer and an electron injection layer, and according to one example, the light-emitting unit may include three or more light-emitting layers. According to one embodiment, the light-emitting layer comprising the light-emitting unit may be three or more. A plurality of light-emitting units may emit the same color or different colors. Additionally, one light-emitting unit may comprise one or more light-emitting layers, and the plurality of light-emitting layers may be of the same or different colors. It may comprise one or more charge generation layers located between each light-emitting unit. The charge generation layer refers to the layer in which holes and electrons are generated when voltage is applied. When light-emitting units are three or more, a charge generation layer may be located between each light-emitting unit. At this time, the plurality of charge generation layers may be the same or different from each other. By arranging the charge generation layer between light-emitting units, current efficiency is increased in each light-emitting unit, and charges can be smoothly distributed. Specifically, the charge generation layer is provided between two adjacent stacks and can serve to drive a tandem organic electroluminescent device using only a pair of anodes and cathodes without a separate internal electrode located between the stacks.

The charge generation layer may be composed of an N-type charge generation layer and a P-type charge generation layer, and the N-type charge generation layer may be doped with an alkali metal, an alkaline earth metal, or a compound of an alkali metal and an alkaline earth metal, The alkali metal may include one selected from the group consisting of Li, Na, K, Rb, Cs, Fr, Yb, and combinations thereof, and the alkaline earth metal may include one selected from the group consisting of Be, Mg, Ca, Sr, Ba, Ra, and combinations thereof. The P-type charge generation layer may be composed of a metal or an organic material doped with a P-type dopant. For example, the metal may be made of one or more alloys selected from the group consisting of Al, Cu, Fe, Pb, Zn, Au, Pt, W, In, Mo, Ni, and Ti. Additionally, commonly used materials may be used as the P-type dopant and host materials used in the P-type doped organic material.

The manufacturing method of the organic electroluminescent device of the present disclosure is not limited, and the manufacturing method of the device example below is only an example and is not limited thereto. One skilled in the art can reasonably describe the manufacturing method of the following device examples by relying on existing technology.

For example, there is no particular limitation on the mixing ratio of the first compound and the second compound, and one skilled in the art can reasonably select it within a certain range depending on existing technology. For example, based on the total weight of the light-emitting layer material, the total weight of the first compound and the second compound accounts for 99.5%-80.0% of the total weight of the light-emitting layer, the weight ratio of the first compound and the second compound is between 1:99 and 99:1, the weight ratio of the first compound and the second compound may be between 20:80 and 99:1, or the weight ratio of the first compound and the second compound may be between 50:50 and 90:10. In the manufacture of devices, when forming a light-emitting layer by co-depositing two or more host materials and a light-emitting material, the two or more types of host materials and light-emitting materials may be placed in different evaporation sources and co-deposited to form a light-emitting layer, a pre-mixed mixture of two or more host materials may be placed on the same evaporation source and then co-deposited with a light-emitting material placed on another evaporation source to form a light-emitting layer. This premixing method can further save evaporation sources. According to one embodiment, the first compound, the second compound, and the light-emitting material of the present disclosure can be placed in different evaporation sources and co-deposited to form a light-emitting layer, or a pre-mixed mixture of the first compound and the second compound may be placed in the same evaporation source and then co-deposited with a light-emitting material placed in another evaporation source to form a light-emitting layer.

In addition, it is possible to produce a display system, for example, a display system for smart phones, tablets, notebooks, PCs, TVs, or cars; or a lighting system, for example an outdoor or indoor lighting system, by using the organic electroluminescent device of the present disclosure.

Hereinafter, the preparation method of the compounds according to the present disclosure and the properties thereof, and the driving voltage and the luminous efficiency of an organic electroluminescent device (OLED) comprising a plurality of host materials according to the present disclosure will be explained in detail with reference to the representative compounds of the present disclosure. However, the following examples only describe the properties of the compound according to the present disclosure and the OLED comprising the same, and the present disclosure is not limited to the following examples.

### Example 1: Preparation of Compound H1-27-D14

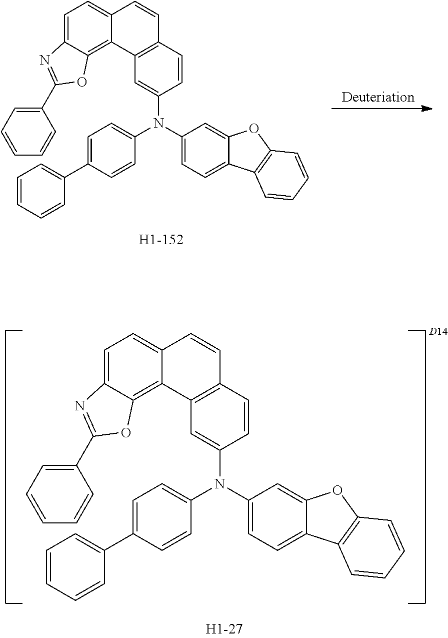

Compound H1-152 (35.0 g, 55.6 mmol), benzene-D6, and 1.4 L of dichlorobenzene were added to a flask, and 70 mL of triflic acid was added at 40° C. After 3 hours, the mixture was cooled to room temperature, added 35 mL of heavy water and stirred for 10 minutes. The mixture was neutralized with aqueous K₃PO₄ solution and the organic layer was extracted with dichloromethane. After the residual moisture was removed using magnesium sulfate, the filtrate was distilled under reduced pressure, and separated by column chromatography to obtain Compound H1-27-D14 (where n is 14) (32.3 g, yield: 92.2%).

### Example 2: Preparation of Compound H1-121-D18

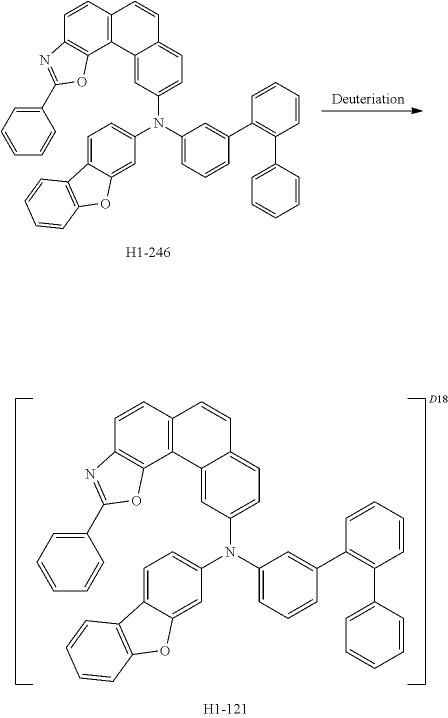

Compound H1-246 (35.0 g, 49.7 mmol), benzene-D6, and 1.4 L of dichlorobenzene were added to a flask, and 70 mL of triflic acid was added at 40° C. After 3 hours, the mixture was cooled to room temperature, added 35 mL of heavy water and stirred for 10 minutes. The mixture was neutralized with aqueous K₃PO₄ solution and the organic layer was extracted with dichloromethane. After the residual moisture was removed using magnesium sulfate, the filtrate was distilled under reduced pressure, and separated by column chromatography to obtain Compound H1-121-D18 (where n is 18) (31 g, yield: 88.5%).

### Example 3: Preparation of Compound H1-33-D15

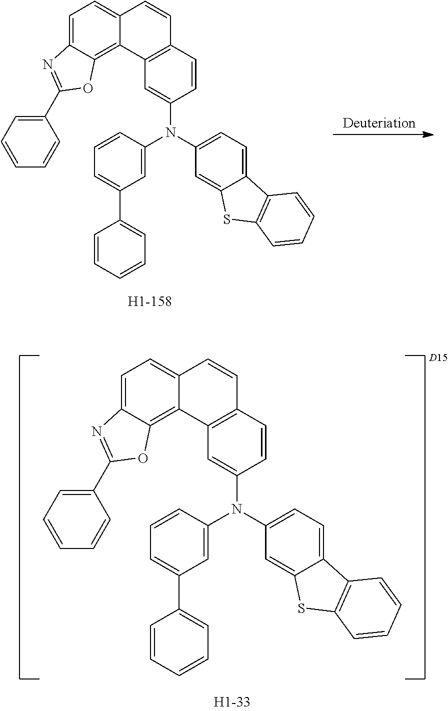

Compound H1-158 (35.0 g, 54.2 mmol), benzene-D6, and 1.4 L of dichlorobenzene were added to a flask, and 70 mL of triflic acid was added at 40° C. After 3 hours, the mixture was cooled to room temperature, added 35 mL of heavy water and stirred for 10 minutes. The mixture was neutralized with aqueous K₃PO₄ solution and the organic layer was extracted with dichloromethane. After the residual moisture was removed using magnesium sulfate, the filtrate was distilled under reduced pressure, and separated by column chromatography to obtain Compound H1-33-D15 (where n is 15) (31 g, yield: 88.5%).

### Example 4: Preparation of Compound H2-1-D11

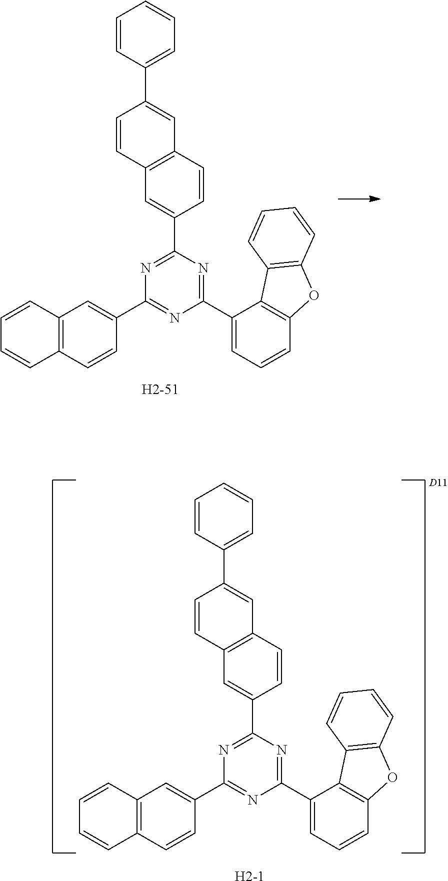

Compound H2-51 (35.0 g, 54.2 mmol), benzene-D6, and 1.4 L of dichlorobenzene were added to a flask, and 60 mL of triflic acid was added at 40° C. After 3 hours, the mixture was cooled to room temperature, added 30 mL of heavy water and stirred for 10 minutes. The mixture was neutralized with aqueous K₃PO₄ solution and the organic layer was extracted with dichloromethane. After the residual moisture was removed using magnesium sulfate, the filtrate was distilled under reduced pressure, and separated by column chromatography to obtain Compound H2-1-D11 (where n is 11) (15 g, yield: 49%).

### Example 5: Preparation of Compound H2-1

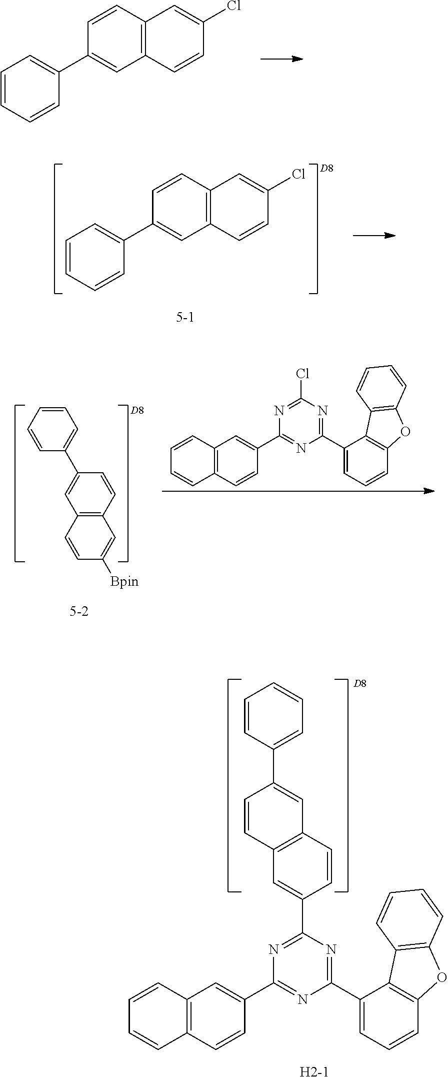

1) Synthesis of Compound 5-1

2-chloro-6-phenylnaphthalene (44.5 g, 186.3 mmol), benzene-D6, and 0.9 L of dichlorobenzene were added to the flask, and 60 mL of triflic acid was added at 60° C. After 12 hours, the mixture was cooled to room temperature, 45 mL of heavy water was added, and stirred for 10 minutes. The mixture was neutralized with aqueous K₃PO₄ solution and the organic layer was extracted with dichloromethane. After the residual moisture was removed using magnesium sulfate, the filtrate was distilled under reduced pressure, and separated by column chromatography to obtain Compound 5-1 (32.6 g, yield: 73%).

2) Synthesis of Compound 5-2

Compound 5-1 (30 g, 122 mmol), bis(pinacolato)diboron (46.5 g, 183 mmol), tris(dibenzylideneacetone)dipalladium (5.6 g, 6.1 mmol), 2-dicyclohexylphosphino-2′,6′-dimethoxybiphenyl (s-phos) (5.0 g, 12.2 mmol), potassium acetate (36.0 g, 366 mmol) and 610 mL of 1,4-dioxane were added to a reaction vessel, and stirred for 3 hours at 150° C. After the reaction was completed, the mixture was cooled to room temperature, and the organic layer was extracted with ethyl acetate. After the organic layer was dried by magnesium sulfate, the solvent was removed by rotary evaporator. The residue was purified by column chromatography to obtain Compound 5-2 (34 g, yield: 82%).

3) Synthesis of Compound H2-1

Compound 5-2 (34 g, 103 mmol), 2-chloro-4-(dibenzo[b,d]furan-1yl)-6-(naphthalen-2-yl)-1,3,5-triazine (42 g, 103 mmol), tetrakis(triphenylphosphine)palladium (5.9 g, 5.2 mmol). calcium carbonate (28.4 g, 206 mmol), 515 mL of toluene, 129 mL of ethanol, 129 mL of distilled water were added to a reaction vessel, and stirred for 8 hours at 120° C. After the reaction was completed, the mixture was added dropwise to methanol, and the resulting solid was filtered. Thereafter, the product was purified by column chromatography to obtain Compound H2-1 (42 g, yield: 70%).

### Example 6: Preparation of Compound H2-38

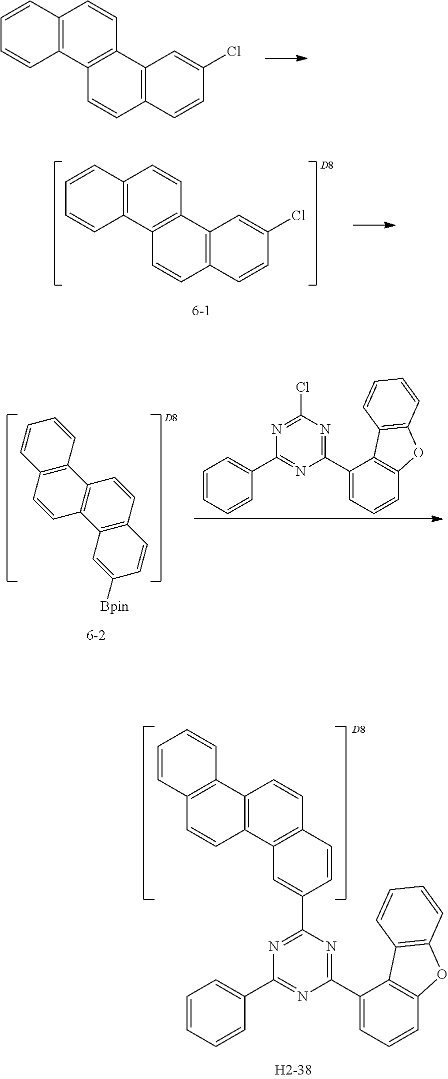

1) Synthesis of Compound 6-1

3-chlorochrysene (30 g, 114 mmol), benzene-D6, and 0.7 L of dichlorobenzene were added to the flask, and 9 mL of triflic acid was added at 50° C. After 12 hours, the mixture was cooled to room temperature, 30 mL of heavy water was added, and stirred for 10 minutes. The mixture was neutralized with aqueous K₃PO₄ solution and the organic layer was extracted with dichloromethane. After the residual moisture was removed using magnesium sulfate, the filtrate was distilled under reduced pressure, and separated by column chromatography to obtain Compound 6-1 (24 g, yield: 80%).

2) Synthesis of Compound 6-2

Compound 6-1 (24 g, 93 mmol), bis(pinacolato)diboron (35.7 g, 140 mmol), tris(dibenzylideneacetone)dipalladium (4.3 g, 4.6 mmol), 2-dicyclohexylphosphino-2′,6′-dimethoxybiphenyl (s-phos) (3.8 g, 9.4 mmol), potassium acetate (27.5 g, 280 mmol) and 460 mL of 1,4-dioxane were added to a reaction vessel, and stirred for 3 hours at 150° C. After the reaction was completed, the mixture was cooled to room temperature, and the organic layer was extracted with ethyl acetate. After the organic layer was dried by magnesium sulfate, the solvent was removed by rotary evaporator. The residue was purified by column chromatography to obtain Compound 6-2 (25 g, yield: 73%).

3) Synthesis of Compound H2-38

Compound 6-2 (10 g, 28 mmol), 2-chloro-4-(dibenzo[b,d]furan-1yl)-6-phenyl-1,3,5-triazine (12 g, 34 mmol), tetrakis(triphenylphosphine)palladium (1.6 g, 1.4 mmol), calcium carbonate (7.8 g, 56 mmol), 141 mL of toluene, 20 mL of ethanol, 30 mL of distilled water were added to a reaction vessel, and stirred for 8 hours at 120° C. After the reaction was completed, the mixture was added dropwise to methanol, and the resulting solid was filtered. Thereafter, the product was purified by column chromatography to obtain Compound H2-38 (12 g, yield: 70%).

### Example 7: Preparation of Compound H2-36

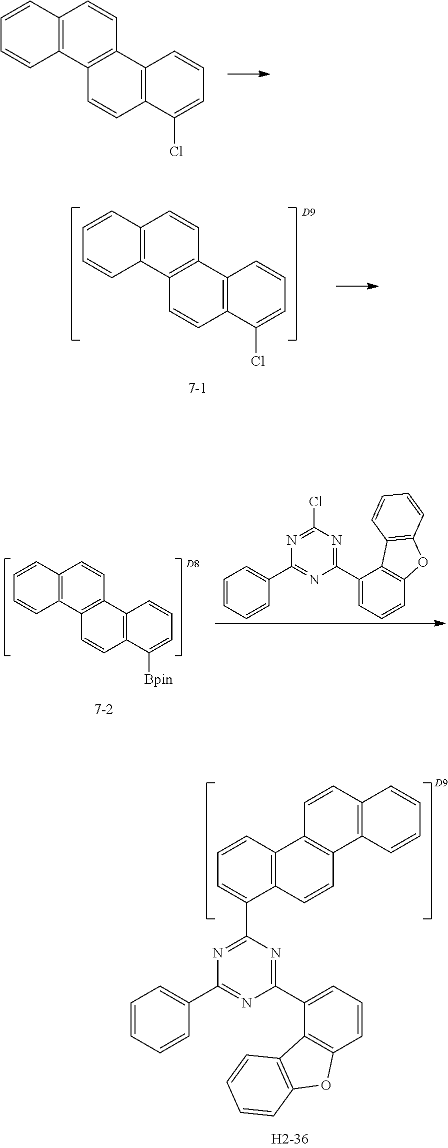

1) Synthesis of Compound 7-1

1-chlorochrysene (28 g, 107 mmol), benzene-D6, and 2.1 L of dichlorobenzene were added to the flask, and 9 mL of triflic acid was added at 50° C. After 12 hours, the mixture was cooled to room temperature, 30 mL of heavy water was added, and stirred for 10 minutes. The mixture was neutralized with aqueous K₃PO₄ solution and the organic layer was extracted with dichloromethane. After the residual moisture was removed using magnesium sulfate, the filtrate was distilled under reduced pressure, and separated by column chromatography to obtain Compound 7-1 (24 g, yield: 85%).

2) Synthesis of Compound 7-2

Compound 7-1 (22.5 g, 86 mmol), bis(pinacolato)diboron (32.6 g, 128 mmol), tris(dibenzylideneacetone)dipalladium (3.9 g, 4.3 mmol), 2-dicyclohexylphosphino-2′,6′-dimethoxybiphenyl (s-phos) (3.5 g, 8.6 mmol), potassium acetate (25.2 g, 257 mmol) and 430 mL of 1,4-dioxane were added to a reaction vessel, and stirred for 3 hours at 150° C. After the reaction was completed, the mixture was cooled to room temperature, and the organic layer was extracted with ethyl acetate. After the organic layer was dried by magnesium sulfate, the solvent was removed by rotary evaporator. The residue was purified by column chromatography to obtain Compound 7-2 (23.5 g, yield: 78%).

3) Synthesis of Compound H2-36

Compound 7-2 (10 g, 28 mmol), 2-chloro-4-(dibenzo[b,d]furan-1yl)-6-phenyl-1,3,5-triazine (12 g, 34 mmol), tetrakis(triphenylphosphine)palladium (1.6 g, 1.4 mmol). calcium carbonate (7.8 g, 56 mmol), 141 mL of toluene, 20 mL of ethanol, 30 mL of distilled water were added to a reaction vessel, and stirred for 8 hours at 120° C. After the reaction was completed, the mixture was added dropwise to methanol, and the resulting solid was filtered. Thereafter, the product was purified by column chromatography to obtain Compound H2-36 (12 g, yield: 70%).

### Example 8: Preparation of Compound H1-35

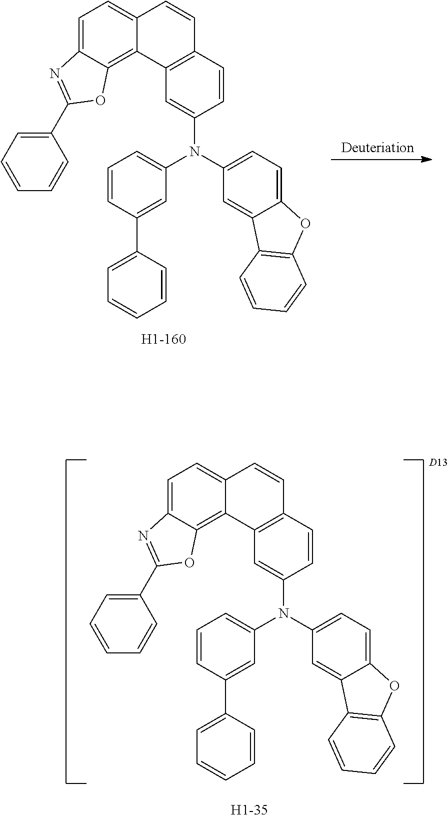

Compound H1-160 was synthesized by selecting one of the deuteration methods disclosed in Korean Patent Publication Nos. 10-2283849, 10-1427457, etc. to obtain Compound H1-35 (1.9 g, yield: 18%).

### Example 9: Preparation of Compound H3-88

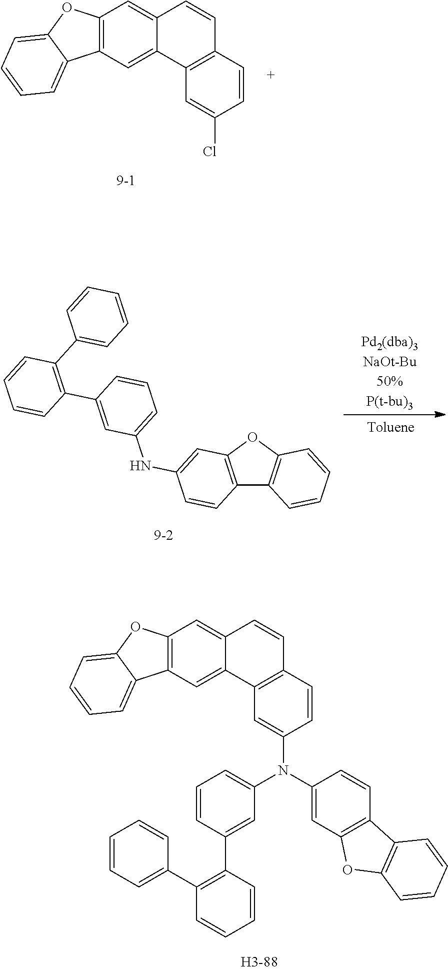 [SMILES: Clc1ccc2ccc3cc4oc5ccccc5c4cc3c2c1 ; c1ccc(-c2ccccc2-c2cccc(Nc3ccc4c(c3)oc3ccccc34)c2)cc1 ; c1ccc(-c2ccccc2-c2cccc(N(c3ccc4c(c3)oc3ccccc34)c3ccc4ccc5cc6oc7ccccc7c6cc5c4c3)c2)cc1]

Compound 9-1 (5.7 g, 18.95 mmol), Compound 9-2 (6 g, 14.58 mmol), Pd₂(dba)₃ (0.66 g, 0.729 mmol), NaOt-Bu (2.1 g, 21.87 mmol), P(t-bu)₃ (50%) (0.7 mL, 1.458 mmol), and 75 mL of toluene were added to a reaction vessel, and stirred under reflux for 1 hour. After the reaction was completed, the mixture was cooled to room temperature, filtered through celite, then the filtrate was distilled under reduced pressure, and separated by column chromatography to obtain Compound H3-88 (4 g, yield: 40%).

| | MW | M.P. |
|---|---|---|
| H3-88 | 677.7 | 256.5° C. |

### Example 10: Preparation of Compound H3-744

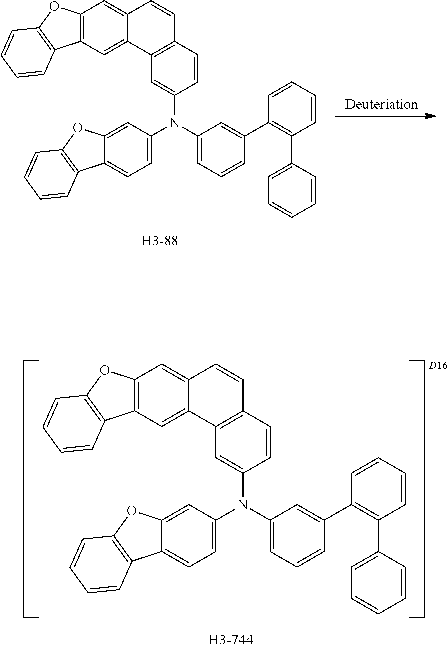

Compound H3-88 was synthesized by selecting one of the deuteration methods disclosed in Korean Patent Publication Nos. 10-2283849, 10-1427457, etc. to obtain Compound H3-744 (4.0 g, yield: 35%, MS: [M+H]⁺=694.2).

### Example 11: Preparation of Compound H3-66

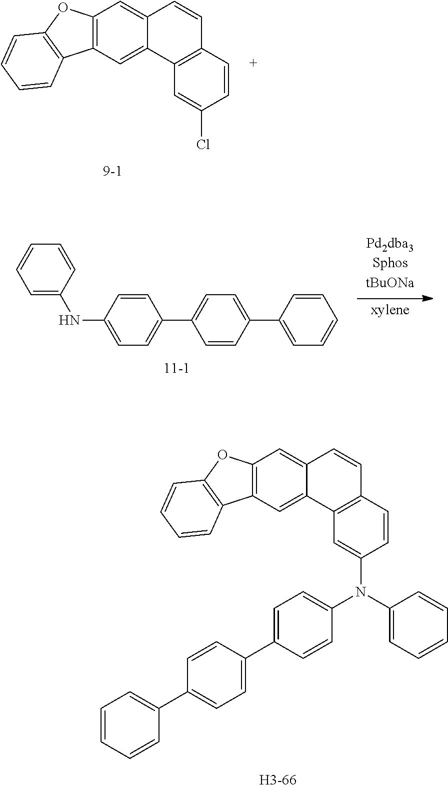 [SMILES: Clc1ccc2ccc3cc4oc5ccccc5c4cc3c2c1 ; c1ccc(Nc2ccc(-c3ccc(-c4ccccc4)cc3)cc2)cc1 ; c1ccc(-c2ccc(-c3ccc(N(c4ccccc4)c4ccc5ccc6cc7oc8ccccc8c7cc6c5c4)cc3)cc2)cc1]

Compound 9-1 (5.9 g, 19.8 mmol), Compound 11-1 (7 g, 21.7 mmol), Pd₂(dba)₃ (906 mg, 0.99 mmol), S-Phos (650 mg, 1.58 mmol), and NaOtBu (2.85 g, 29.7 mmol) were dissolved in 100 mL of xylene and stirred under reflux for 30 minutes at 160° C. After the reaction was completed, the mixture was cooled to room temperature, filtered through celite to obtain a solid, and purified by column chromatography to obtain Compound H3-66 (2.4 g, yield: 20.6%).

| | MW | M.P. |
|---|---|---|
| H3-66 | 587.72 | 253.3° C. |

### Example 12: Preparation of Compound H3-746

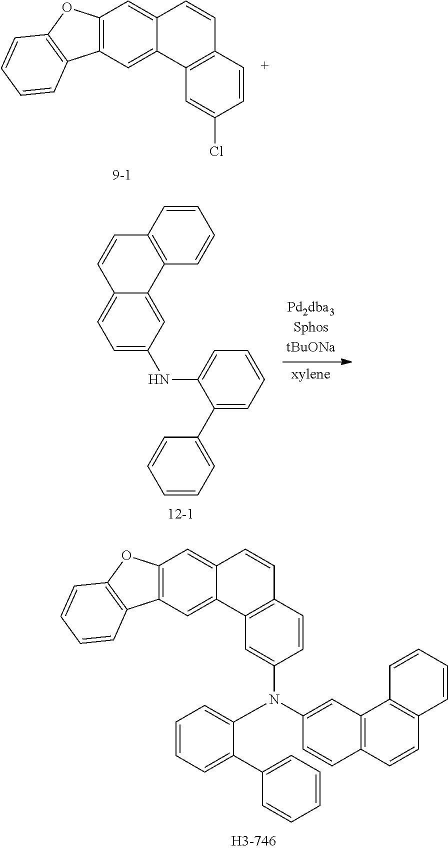 [SMILES: Clc1ccc2ccc3cc4oc5ccccc5c4cc3c2c1 ; c1ccc(-c2ccccc2Nc2ccc3ccc4ccccc4c3c2)cc1 ; c1ccc(-c2ccccc2N(c2ccc3ccc4ccccc4c3c2)c2ccc3ccc4cc5oc6ccccc6c5cc4c3c2)cc1]

Compound 9-1 (6.3 g, 21 mmol), Compound 12-1 (8 g, 23.1 mmol), Pd₂(dba)₃ (956 mg, 1.05 mmol), S-Phos (689 mg, 1.68 mmol), and NaOtBu (3.03 g, 31.5 mmol) were dissolved in 105 mL of xylene and stirred under reflux for 30 minutes at 160° C. After the reaction was completed, the mixture was cooled to room temperature, filtered through celite to obtain a solid, and purified by column chromatography to obtain Compound H3-746 (3.5 g, yield: 27%).

| | MW | M.P. |
|---|---|---|
| H3-746 | 611.74 | 149° C. |

### Example 13: Preparation of Compound H3-746

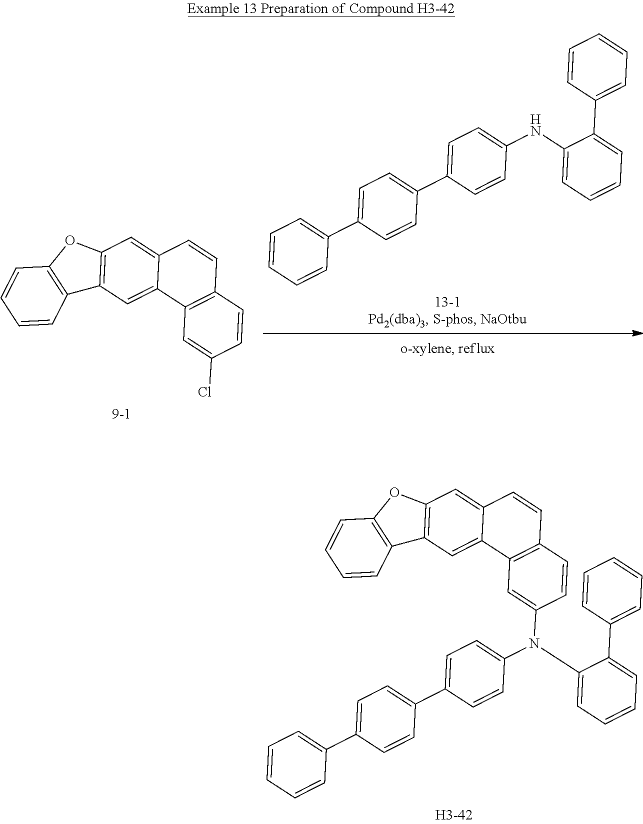 [SMILES: Clc1ccc2ccc3cc4oc5ccccc5c4cc3c2c1 ; c1ccc(-c2ccc(-c3ccc(Nc4ccccc4-c4ccccc4)cc3)cc2)cc1 ; c1ccc(-c2ccc(-c3ccc(N(c4ccc5ccc6cc7oc8ccccc8c7cc6c5c4)c4ccccc4-c4ccccc4)cc3)cc2)cc1]

Compound 9-1 (5.08 g, 16.77 mmol), Compound 13-1 (7.0 g, 17.66 mmol), Pd₂(dba)₃ (0.77 g, 0.84 mmol), S-phos (0.7 g, 1.68 mmol), and NaOtBu (2.42 g, 25.15 mmol) were dissolved in 84 mL of o-xylene and stirred under reflux for 4 hours at 160° C. After the reaction was completed, the mixture was cooled to room temperature, the layers were separated (EA/H₂O), filtered through celite, then filtered through silica to obtain a solid. Then, the solid was filtered to obtain Compound H3-42 (4.7 g, yield: 40%).

| | MW | M.P. |
|---|---|---|
| H3-42 | 663.8 | 257° C. |

### Example 14: Preparation of Compound H3-700

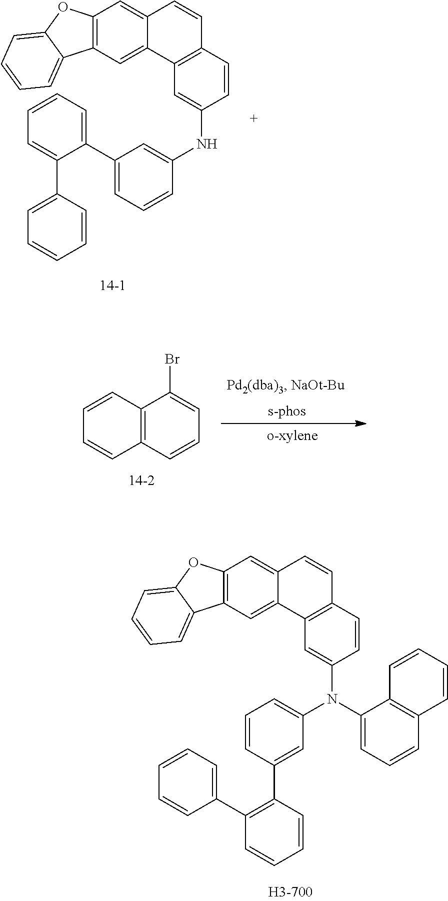 [SMILES: c1ccc(-c2ccccc2-c2cccc(Nc3ccc4ccc5cc6oc7ccccc7c6cc5c4c3)c2)cc1 ; Brc1cccc2ccccc12 ; c1ccc(-c2ccccc2-c2cccc(N(c3ccc4ccc5cc6oc7ccccc7c6cc5c4c3)c3cccc4ccccc34)c2)cc1]

Compound 14-1 (7.4 g, 14.48 mmol), Compound 14-2 (1.68 mL, 12.07 mmol), tris(dibenzylideneacetone)dipalladium(0) (0.55 g, 0.603 mmol), NaOtBu (1.74 g, 18.10 mmol), s-phos (0.5 g, 1.207 mmol) were dissolved in 60 mL of o-xylene and stirred under reflux for 3 hours at 160° C. After the reaction was completed, the mixture was cooled to room temperature, filtered through celite, the filtrate was distilled under reduced pressure, and separated by column chromatography to obtain Compound H3-700 (4.5 g, yield: 58%).

| | MW | M.P. |
|---|---|---|
| H3-700 | 637.7 | 197° C. |

### Example 15: Preparation of Compound H3-724

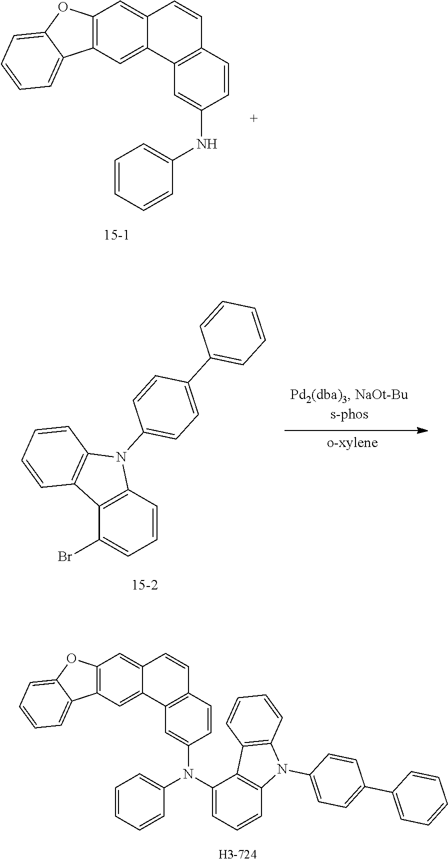 [SMILES: c1ccc(Nc2ccc3ccc4cc5oc6ccccc6c5cc4c3c2)cc1 ; Brc1cccc2c1c1ccccc1n2-c1ccc(-c2ccccc2)cc1 ; c1ccc(-c2ccc(-n3c4ccccc4c4c(N(c5ccccc5)c5ccc6ccc7cc8oc9ccccc9c8cc7c6c5)cccc43)cc2)cc1]

Compound 15-1 (5 g, 13.91 mmol), Compound 15-2 (6.65 mL, 16.69 mmol), tris(dibenzylideneacetone)dipalladium(0) (0.63 g, 0.695 mmol), NaOt-Bu (2 g, 20.86 mmol), and s-phos (0.57 g, 1.391 mmol) were dissolved in 70 mL of o-xylene and stirred under reflux for 24 hours at 160° C. After the reaction was completed, the mixture was cooled to room temperature, filtered through celite, the filtrate was distilled under reduced pressure, and separated by column chromatography to obtain Compound H3-724 (4 g, yield: 42%).

| | MW | M.P. |
|---|---|---|
| H3-724 | 676.8 | 218.3° C. |

### Device Examples 1 and 2: Producing OLEDs Comprising the Plurality of Host Materials According to the Present Disclosure

An OLED according to the present disclosure was produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 shown in Table 6 below was introduced into a cell of the vacuum vapor deposition apparatus, and Compound HT-1 was introduced into another cell of the vacuum vapor deposition apparatus. The two materials were evaporated at different rates, and Compound HI-1 was deposited in a doping amount of 3 wt % based on the total amount of Compound HI-1 and Compound HT-1 to form a hole injection layer having a thickness of 10 nm on the ITO substrate. Next, Compound HT-1 was deposited on the hole injection layer to form a first hole transport layer having a thickness of 80 nm. The Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: each of the first host compound and the second host compound disclosed in Table 1 below were introduced into two cells of the vacuum vapor deposition apparatus as hosts, and Compound D-150 was introduced into another cell as a dopant. The two host materials were evaporated at a different rate of 1:1 and the dopant material was simultaneously evaporated at a different rate, and the dopant was deposited in a doping amount of 3 wt % based on the total amount of the host and the dopant to form a light-emitting layer having a thickness of 40 nm on the second hole transport layer. Compound ET-1 and Compound EI-1 were evaporated in a weight ratio of 50:50 to form an electron transport layer having a thickness of 35 nm on the light-emitting layer. After depositing Compound EI-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

### Comparative Example 1: Producing an OLED Comprising a Comparative Compound as a Host

An OLED was produced in the same manner as in Device Example 1, except that the second host compound in Table 1 below was used as the host of the light-emitting layer.

The light-emitting color and the time taken for luminance to reduce from 100% to 90% at a luminance of 15,000 nit (lifetime; T90) of the OLEDs produced in Device Examples 1 and 2 and Comparative Example 1 are shown in Table 1 below.

TABLE 1

| | | | | Life- |
|---|---|---|---|---|
| | First | Second | Light- | time |
| | Host | Host | Emitting | (T90) |
| | Compound | Compound | Color | [hr] |
| Device Example 1 | H1-152 | H2-1 | Red | 778 |
| Device Example 2 | H1-27 | H2-1 | Red | 1050 |
| Comparative Example 1 | H1-152 | H2-51 | Red | 673 |

From Table 1 above, it can be confirmed that the lifetime of an organic electroluminescent device comprising the plurality of host materials including a specific combination of compounds according to the present disclosure is significantly improved compared to a conventional organic electroluminescent device comprising a host material not containing deuterium.

### Device Example 3: Producing an OLED Comprising the Plurality of Host Materials According to the Present Disclosure

An OLED was produced in the same manner as in Device Example 2, except that after forming the Compound HT-3 in Table 7 below to a thickness of 45 nm as a second hole transport layer, the Compound HT-4 in Table 7 below was deposited to a thickness of 15 nm as a third hole transport layer thereon, the thickness of the electron transport layer was reduced to 30 nm, and the Compound BF-1 in Table 7 was deposited to a thickness of 5 nm between the light-emitting layer and the electron transport layer to form an electron buffer layer, and Compound ET-2 was used in the electron transport layer.

### Comparative Example 2: Producing an OLED Comprising a Comparative Compound as a Host

An OLED was produced in the same manner as in Device Example 3, except that a first host and a second host in Table 2 below were used as the host of the light-emitting layer.

The light-emitting color and the time taken for luminance to reduce from 100% to 95% at a luminance of 10,000 nit (lifetime: T95) of the OLEDs produced in Device Example 3 and Comparative Example 2 are shown in Table 2 below.

TABLE 2

| | | | | Life- |
|---|---|---|---|---|
| | First | Second | Light- | time |
| | Host | Host | Emitting | (T95) |
| | Compound | Compound | Color | [hr] |
| Device Example 3 | H1-27 | H2-51 | Red | 892 |
| Comparative Example 2 | H1-152 | H2-51 | Red | 692 |

From Table 2 above, it can be confirmed that the lifetime of an organic electroluminescent device comprising a plurality of host materials according to the present disclosure exhibits longer lifetime properties compared to a conventional organic electroluminescent device comprising a host material not containing deuterium.

### Device Examples 4 to 6: Producing OLEDs Comprising the Plurality of Host Materials According to the Present Disclosure

An OLED was produced in the same manner as in Device Example 1, except that Compound HT-5 in Table 7 below was used as a second hole transport layer and Compound D-39 was used as a dopant.

### Comparative Examples 3 to 5: Producing OLEDs Comprising a Comparative Compound as a Host

An OLED was produced in the same manner as in Device Example 4, except that a first host and a second host in Table 3 below were used as the host of the light-emitting layer.

The light-emitting color and the time taken for luminance to reduce from 100% to 95% at a luminance of 10,000 nit (lifetime: T95) of the OLEDs produced in Device Examples 4 to 6 and Comparative Examples 3 to 5 are shown in Table 3 below.

TABLE 3

| | | | | Life- |
|---|---|---|---|---|
| | First | Second | Light- | time |
| | Host | Host | Emitting | (T95) |
| | Compound | Compound | Color | [hr] |
| Device Example 4 | H1-35 | H2-88 | Red | 490 |
| Comparative Example 3 | H1-160 | H2-88 | Red | 380 |
| Device Example 5 | H1-121 | H2-88 | Red | 627 |
| Comparative Example 4 | H1-246 | H2-88 | Red | 514 |
| Device Example 6 | H1-33 | H2-88 | Red | 653 |
| Comparative Example 5 | H1-158 | H2-88 | Red | 449 |

From Table 3 above, it can be confirmed that the lifetime of an organic electroluminescent device comprising a plurality of host materials according to the present disclosure exhibits longer lifetime characteristics compared to a conventional organic electroluminescent device comprising a host material not containing deuterium.

### Device Examples 7 and 8: Producing OLEDs Comprising the Plurality of Host Materials According to the Present Disclosure

An OLED was produced in the same manner as in Device Example 1, except that the ratio of the first host and the second host of the light-emitting layer host material was deposited at a ratio of 4:6 and Compound 0-39 in Table 7 below was used as a dopant.

### Comparative Example 6: Producing an OILED Comprising a Comparative Compound as a Host

An OLED was produced in the same manner as in Device Example 7, except that a first host and a second host in Table 4 below were used as the host of the light-emitting layer.

The light-emitting color and the time taken for luminance to reduce from 100% to 95% at a luminance of 10,000 nit (lifetime: T95) of the OLEDs produced in Device Examples 7 and 8 and Comparative Example 6 are shown in Table 4 below.

TABLE 4

| | | | | Life- |
|---|---|---|---|---|
| | First | Second | Light- | time |
| | Host | Host | Emitting | (T95) |
| | Compound | Compound | Color | [hr] |
| Device Example 7 | H1-33 | H2-51 | Red | 190 |
| Device Example 8 | H1-158 | H2-1 | Red | 153 |
| Comparative Example 6 | H1-158 | H2-51 | Red | 138 |

From Table 4 above, it can be confirmed that the lifetime of an organic electroluminescent device comprising a plurality of host materials according to the present disclosure exhibits longer lifetime properties compared to a conventional organic electroluminescent device comprising a host material not containing deuterium.

### Device Examples 9 to 11: Producing OLEDs Comprising the Plurality of Host Materials According to the Present Disclosure

An OLED was produced in the same manner as in Device Example 7, except that the ratio of the first host, the second host, and the third host of the light-emitting layer host material was deposited at a ratio of 2:6:2.

### Comparative Example 7: Producing an OLED Comprising a Comparative Compound as a Host

An OLED was produced in the same manner as in Device Example 9, except that a first host, a second host, and a third host in Table 5 below were used as the host of the light-emitting layer.

The light-emitting color and the time taken for luminance to reduce from 100% to 95% at a luminance of 10,000 nit (lifetime: T95) of the OLEDs produced in Device Examples 9 to 11 and Comparative Example 7 are shown in Table 5 below.

TABLE 5

| | First | Second | Third | | Life- |
|---|---|---|---|---|---|
| | Host | Host | Host | Light- | time |
| | Com- | Com- | Com- | Emitting | (T95) |
| | pound | pound | pound | Color | [hr] |
| Device Example 9 | H1-33 | H2-51 | H3-88 | Red | 191 |
| Device Example 10 | H1-158 | H2-51 | H3-744 | Red | 169 |
| Device Example 11 | H1-158 | H2-1 | H3-744 | Red | 200 |
| Comparative | H1-158 | H2-51 | H3-88 | Red | 152 |
| Example 7 | | | | | |

From Table 5 above, it can be confirmed that the lifetime of an organic electroluminescent device comprising a plurality of host materials according to the present disclosure exhibits longer lifetime properties compared to a conventional organic electroluminescent device comprising a host material not containing deuterium.

### Device Examples 12 and 13: Producing OLEDs Comprising the Plurality of Host Materials According to the Present Disclosure

An OLED was produced in the same manner as in Device Example 7, except that Compound D-151 in Table 7 below was used as a dopant.

### Comparative Examples 8 and 9: Producing OLEDs Comprising a Comparative Compound as a Host

An OLED was produced in the same manner as in Device Example 7, except that a first host and a second host in Table 6 below were used as the host of the light-emitting layer.

The light-emitting color and the time taken for luminance to reduce from 100% to 95% at a luminance of 10,000 nit (lifetime: T95) of the OLEDs produced in Device Examples 12 and 13 and Comparative Examples 8 and 9 are shown in Table 6 below.

TABLE 6

| | | | | Life- |
|---|---|---|---|---|
| | First | Second | Light- | time |
| | Host | Host | Emitting | (T95) |
| | Compound | Compound | Color | [hr] |
| Device Example 12 | H1-138 | H2-245 | Red | 306 |
| Comparative Example 8 | H1-138 | H2-165 | Red | 174 |
| Device Example 13 | H1-138 | H2-176 | Red | 475 |
| Comparative Example 9 | H1-138 | H2-171 | Red | 424 |

From Table 6 above, it can be confirmed that the lifetime of an organic electroluminescent device comprising a plurality of host materials according to the present disclosure exhibits longer lifetime properties compared to a conventional organic electroluminescent device comprising a host material not containing deuterium.

The compounds used in the Device Examples and the Comparative Examples are shown in Table 7 below.

TABLE 7

| | |
|---|---|
| Hole Injection Layer/ Hole Transport Layer | 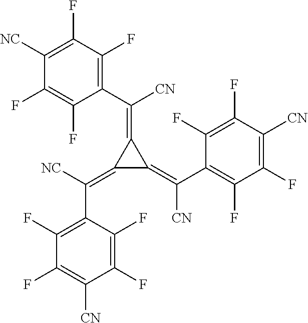 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 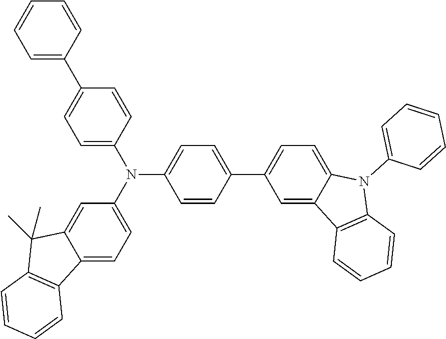 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] |
| | HT-1 |
| | 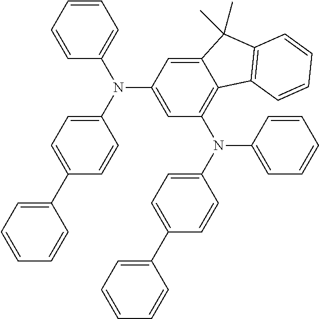 [SMILES: CC1(C)c2ccccc2-c2c(N(c3ccccc3)c3ccc(-c4ccccc4)cc3)cc(N(c3ccccc3)c3ccc(-c4ccccc4)cc3)cc21] |
| | HT-2 |
| | 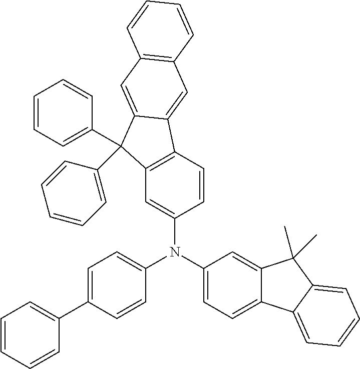 [SMILES: CC1(C)c2ccccc2-c2ccc(N(c3ccc(-c4ccccc4)cc3)c3ccc4c(c3)C(c3ccccc3)(c3ccccc3)c3cc5ccccc5cc3-4)cc21] |
| | HT-3 |
| | 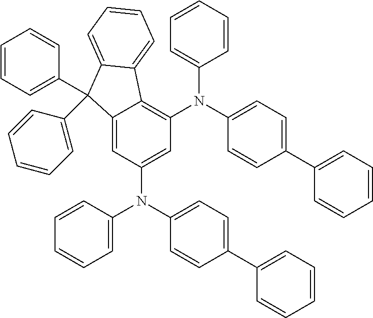 [SMILES: c1ccc(-c2ccc(N(c3ccccc3)c3cc(N(c4ccccc4)c4ccc(-c5ccccc5)cc4)c4c(c3)C(c3ccccc3)(c3ccccc3)c3ccccc3-4)cc2)cc1] |
| | HT-4 |
| | 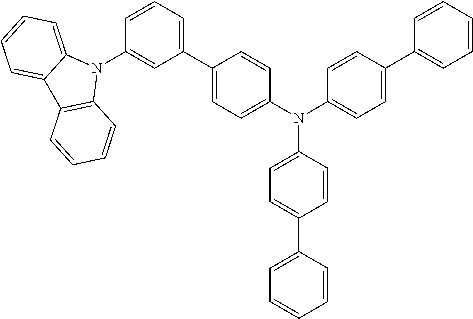 [SMILES: c1ccc(-c2ccc(N(c3ccc(-c4ccccc4)cc3)c3ccc(-c4cccc(-n5c6ccccc6c6ccccc65)c4)cc3)cc2)cc1] |
| | HT-5 |
| Light-Emitting Layer | 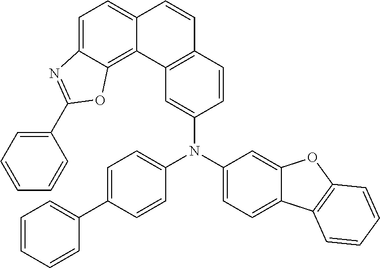 [SMILES: c1ccc(-c2ccc(N(c3ccc4c(c3)oc3ccccc34)c3ccc4ccc5ccc6nc(-c7ccccc7)oc6c5c4c3)cc2)cc1] |
| | H1-152 |
| | 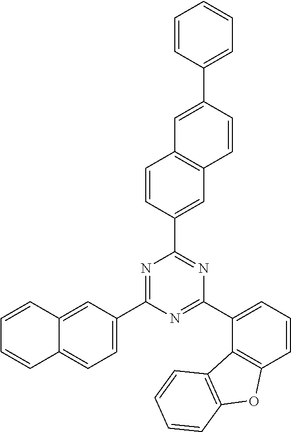 [SMILES: c1ccc(-c2ccc3cc(-c4nc(-c5ccc6ccccc6c5)nc(-c5cccc6oc7ccccc7c56)n4)ccc3c2)cc1] |
| | H2-51 |
| | 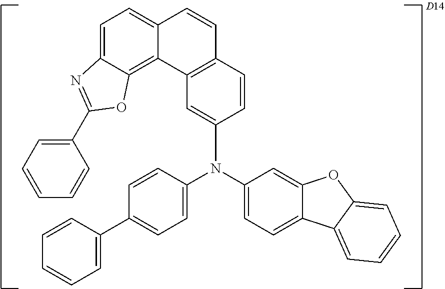 |
| | H1-27 |
| | 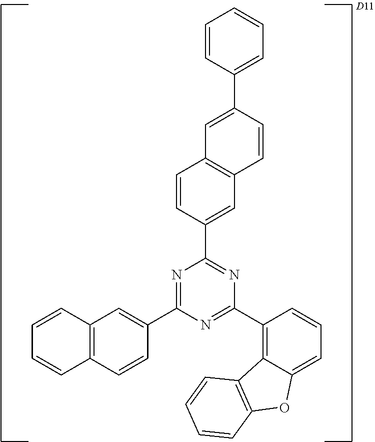 |
| | H2-1 |
| | 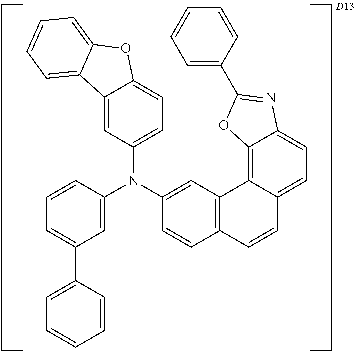 |
| | H1-35 |
| | 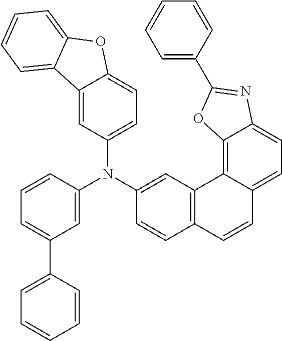 [SMILES: c1ccc(-c2cccc(N(c3ccc4oc5ccccc5c4c3)c3ccc4ccc5ccc6nc(-c7ccccc7)oc6c5c4c3)c2)cc1] |
| | H1-160 |
| | 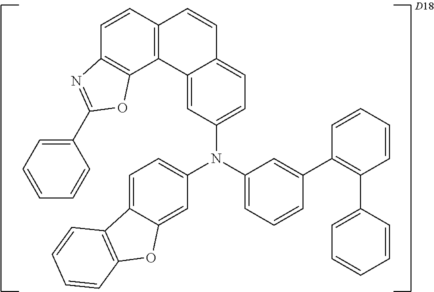 |
| | H1-121 |
| | 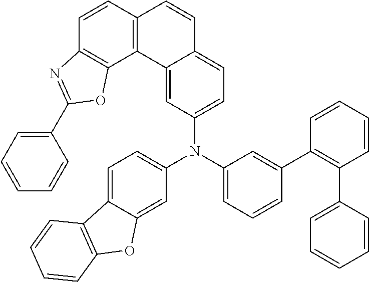 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1] |
| | H1-246 |
| | 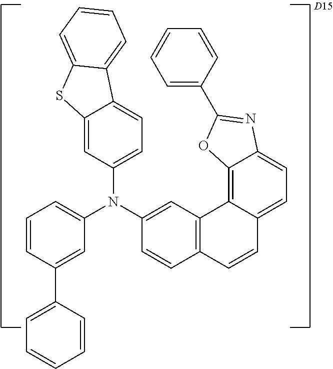 |
| | H1-33 |
| | 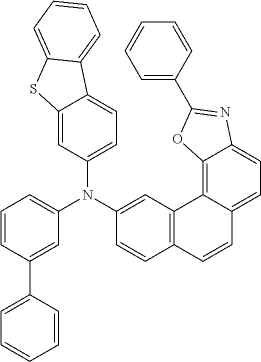 [SMILES: c1ccc(-c2cccc(N(c3ccc4c(c3)sc3ccccc34)c3ccc4ccc5ccc6nc(-c7ccccc7)oc6c5c4c3)c2)cc1] |
| | H1-158 |
| | 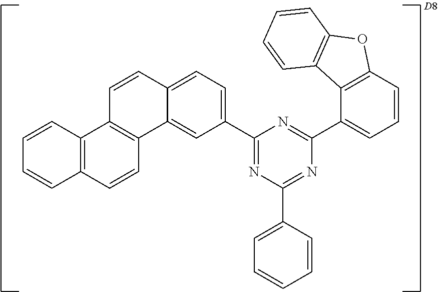 |
| | H2-38 |
| | 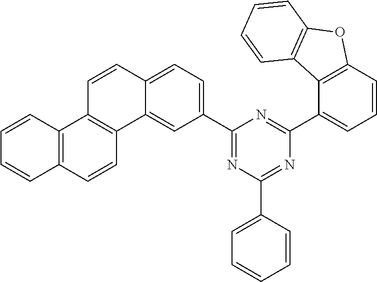 [SMILES: c1ccc(-c2nc(-c3ccc4ccc5c6ccccc6ccc5c4c3)nc(-c3cccc4oc5ccccc5c34)n2)cc1] |
| | H2-88 |
| | 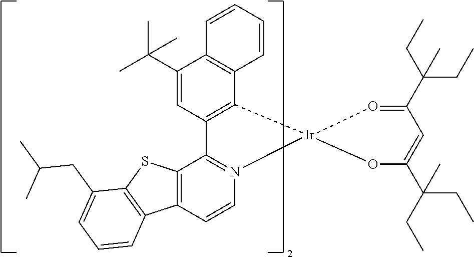 |
| | D-150 |
| | 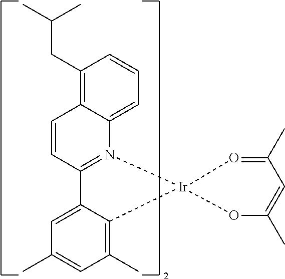 |
| | D-39 |
| | 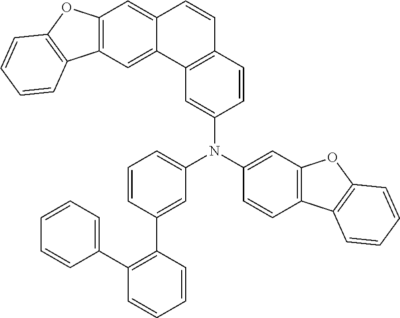 [SMILES: c1ccc(-c2ccccc2-c2cccc(N(c3ccc4c(c3)oc3ccccc34)c3ccc4ccc5cc6oc7ccccc7c6cc5c4c3)c2)cc1] |
| | H3-88 |
| | 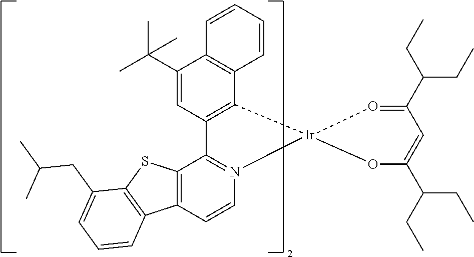 |
| | D-151 |
| | 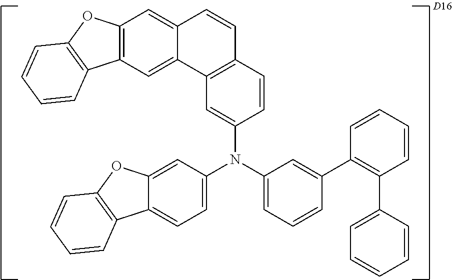 |
| | H3-744 |
| | 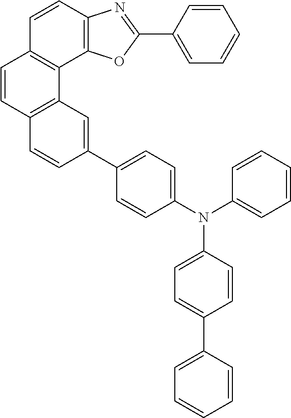 [SMILES: c1ccc(-c2ccc(N(c3ccccc3)c3ccc(-c4ccc5ccc6ccc7nc(-c8ccccc8)oc7c6c5c4)cc3)cc2)cc1] |
| | H1-138 |
| | 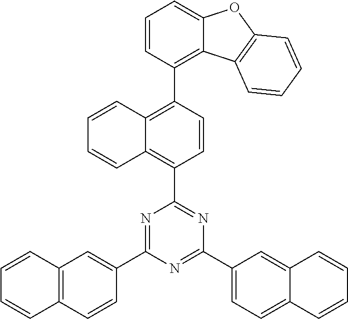 [SMILES: c1ccc2cc(-c3nc(-c4ccc5ccccc5c4)nc(-c4ccc(-c5cccc6oc7ccccc7c56)c5ccccc45)n3)ccc2c1] |
| | H2-165 |
| | 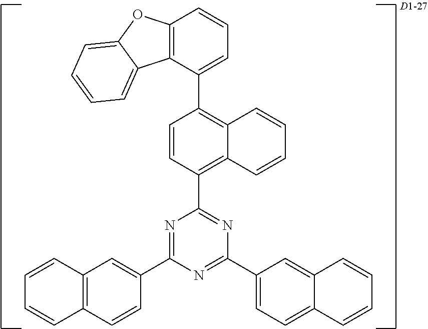 |
| | H2-245 |
| | 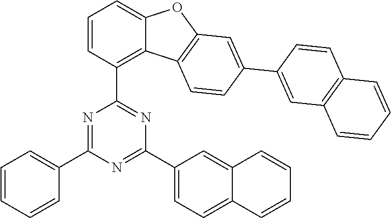 [SMILES: c1ccc(-c2nc(-c3ccc4ccccc4c3)nc(-c3cccc4oc5cc(-c6ccc7ccccc7c6)ccc5c34)n2)cc1] |
| | H2-171 |
| | 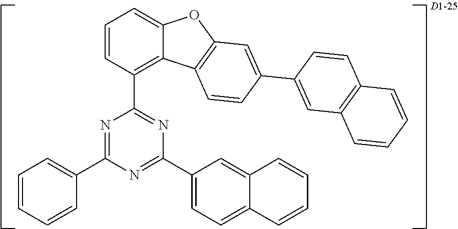 |
| | H2-176 |
| Electron Buffer Layer/ Electron Transport Layer/ Electron Injection Layer | 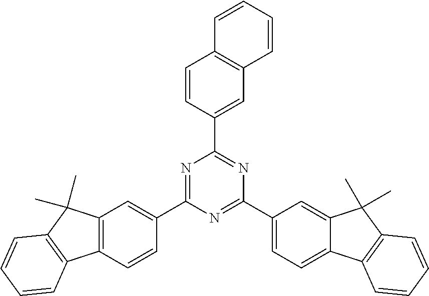 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | ET-1 |
| | 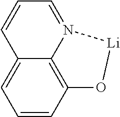 |
| | EI-1 |
| | 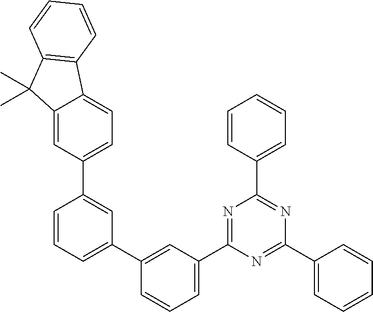 [SMILES: CC1(C)c2ccccc2-c2ccc(-c3cccc(-c4cccc(-c5nc(-c6ccccc6)nc(-c6ccccc6)n5)c4)c3)cc21] |
| | BF-1 |
| | 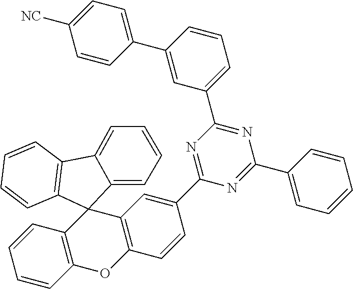 [SMILES: N#Cc1ccc(-c2cccc(-c3nc(-c4ccccc4)nc(-c4ccc5c(c4)C4(c6ccccc6O5)c5ccccc5-c5ccccc54)n3)c2)cc1] |
| | ET-2 |

1. A plurality of host materials comprising a first host material comprising at least one compound represented by the following formula 1 and a second host material comprising at least one compound represented by the following formula 2, wherein at least one of the first host material and the second host material comprises deuterium: 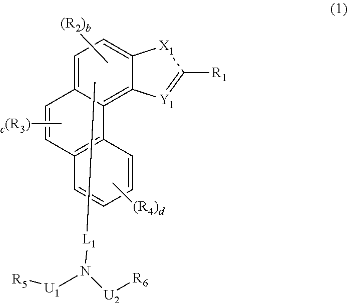
in formula 1,
X₁ and Y₁ each independently represent —N═, —NR₇—, —O— or —S—, with a proviso that one of X₁ and Y₁ represents —N═, and the other of X₁ and Y₁ represents —NR—, —O— or —S—;
R₁ represents a substituted or unsubstituted (C6-C30)aryl, or a substituted or unsubstituted (3- to 30-membered)heteroaryl;
R₂ to R₇ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino, or may be linked to an adjacent substituent(s) to form a ring(s);
L₁, U₁, and U₂ each independently represent a single bond, a substituted or unsubstituted (C6-C30)arylene, or a substituted or unsubstituted (3- to 30-membered)heteroarylene; and
b and c each independently represent an integer of 1 or 2, d represents an integer of 1 to 4, where if b to d represent an integer of 2 or more, each of R₂ to each of R₄ may be the same as or different from each other; 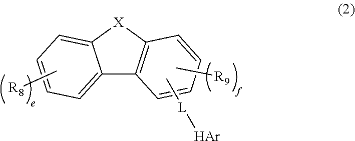
in formula 2,
X represents O or S;
HAr represents a substituted or unsubstituted (3- to 30-membered)heteroaryl, including at least one nitrogen atom;
L represents a single bond, a substituted or unsubstituted (C6-C30)arylene, or a substituted or unsubstituted (3- to 30-membered)heteroarylene;
R₈ and R₉ each independently represent hydrogen, deuterium, a substituted or unsubstituted (C6-C30)aryl, a substituted or unsubstituted (3- to 30-membered)heteroaryl, or —SiR₂₁R₂₂R₂₃;
R₂₁ to R₂₃ each independently represent a substituted or unsubstituted (C1-C30)alkyl, a substituted or unsubstituted (C6-C30)aryl, or a substituted or unsubstituted (3- to 30-membered)heteroaryl; and
e represents an integer of 1 to 4, and f represents an integer of 1 to 3, where if e and f represent an integer of 2 or more, each of R₈ and each of R₉ may be the same as or different from each other.

2. The plurality of host materials according to claim 1, wherein formula 1 is represented by at least one of the following formulas 1-1 to 1-4: 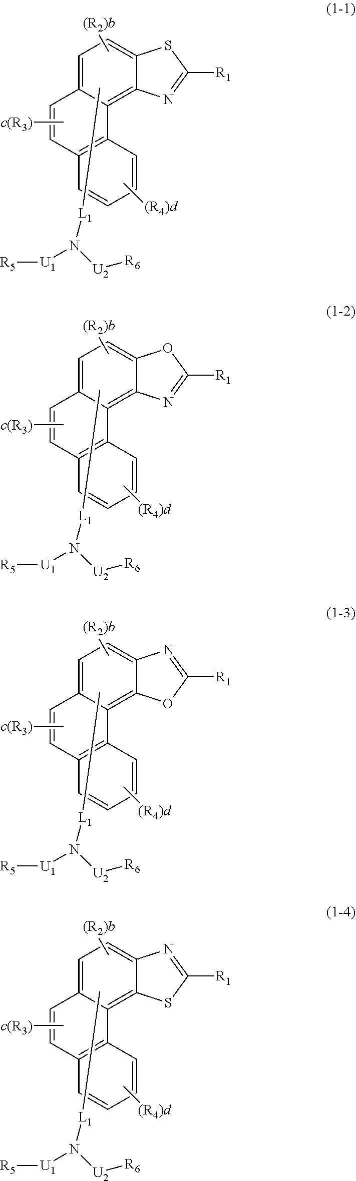
in formulas 1-1 to 1-4,
R₁ to R₆, L₁, U₁, U₂, and b to d are as defined in claim 1.

3. The plurality of host materials according to claim 1, wherein R₅ and R₆ in formula 1 each independently represent a substituted or unsubstituted phenyl, a substituted or unsubstituted naphthyl, a substituted or unsubstituted biphenyl, a substituted or unsubstituted terphenyl, a substituted or unsubstituted phenanthrenyl, a substituted or unsubstituted fluorenyl, a substituted or unsubstituted benzofluorenyl, a substituted or unsubstituted triphenylenyl, a substituted or unsubstituted spirobifluorenyl, a substituted or unsubstituted pyridiyl, a substituted or unsubstituted triazinyl, a substituted or unsubstituted pyrimidinyl, a substituted or unsubstituted quinolyl, a substituted or unsubstituted quinazolinyl, a substituted or unsubstituted quinoxalinyl, a substituted or unsubstituted benzoquinazolinyl, a substituted or unsubstituted benzoquinoxalinyl, a substituted or unsubstituted benzopuropyridinyl, a substituted or unsubstituted benzopuropyrimidinyl, a substituted or unsubstituted carbazolyl, a substituted or unsubstituted dibenzothiophenyl, a substituted or unsubstituted benzothiophenyl, a substituted or unsubstituted dibenzofuranyl, a substituted or unsubstituted benzofuranyl, a substituted or unsubstituted naphthyridinyl, a substituted or unsubstituted benzonaphthofuranyl, a substituted or unsubstituted benzonaphthothiophenyl, or a substituted or unsubstituted triphenylsilyl.

4. The plurality of host materials according to claim 1, wherein formula 2 is represented by the following formula 2-1: 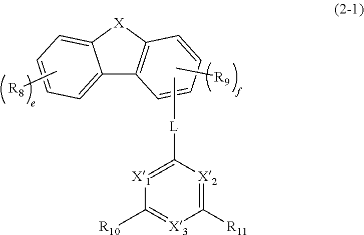
in formula 2-1,
X′₁ to X′₃ each independently represent CR′ or N, with a proviso that at least two of X′₁ to X′₃ represent N;
R′ represents hydrogen or deuterium;
R₁₀ and R₁₁ each independently represent a substituted or unsubstituted (C6-C30)aryl, or a substituted or unsubstituted (3- to 30-membered)heteroaryl; and
X, L, R₃, R₉, e and f are as defined in claim 1.

5. The plurality of host materials according to claim 4, wherein formula 2-1 is represented by at least one of the following formulas 2-1-1 to 2-1-4: 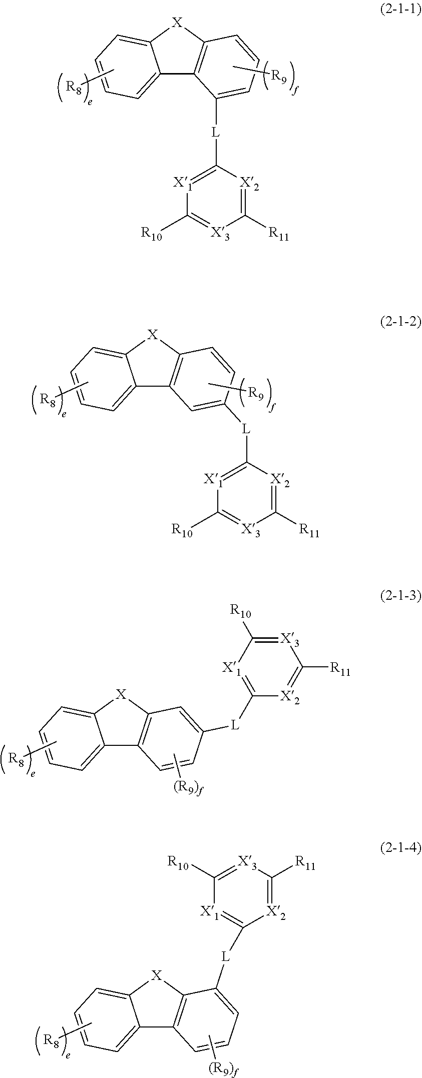
in formulas 2-1-1 to 2-1-4,
X, X′₁ to X′₃, R₅ to R₁₁, L, e and f are as defined in claim 4.

6. The plurality of host materials according to claim 1, wherein the substituted aryl, the substituted heteroaryl, the substituted arylene, the substituted heteroarylene, the substituted alkyl, the substituted cycloalkyl, the substituted alkoxy, the substituted trialkylsilyl, the substituted dialkylarylsilyl, the substituted alkyldiarylsilyl, the substituted triarylsilyl, the substituted fused ring group of aliphatic ring and aromatic ring, the substituted mono- or di-alkylamino, the substituted mono- or di-alkenylamino, the substituted alkylalkenylamino, the substituted mono- or di-arylamino, the substituted alkylarylamino, the substituted mono- or di-heteroarylamino, the substituted alkylheteroarylamino, the substituted alkenylarylamino, the substituted alkenylheteroarylamino, and the substituted arylheteroarylamino are substituted with at least one selected from the group consisting of deuterium; a halogen; a cyano; a carboxyl; a nitro; a hydroxy; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of deuterium and a (C6-C30)aryl(s); a (C6-C30)aryl unsubstituted or substituted with at least one of deuterium and a (C6-C30)aryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30)aliphatic ring(s) and a (C6-C30)aromatic ring(s); an amino; a mono- or di-(C1-C30)alkylamino; a mono- or di-(C2-C30)alkenylamino; a (C1-C30)alkyl(C2-C30)alkenylamino; a mono- or di-(C6-C30)arylamino; a (C1-C30)alkyl(C6-C30)arylamino; a mono- or di-(3- to 30-membered)heteroarylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino, a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

7. The plurality of host materials according to claim 1, wherein the compound represented by formula 1 is selected from the following compounds:

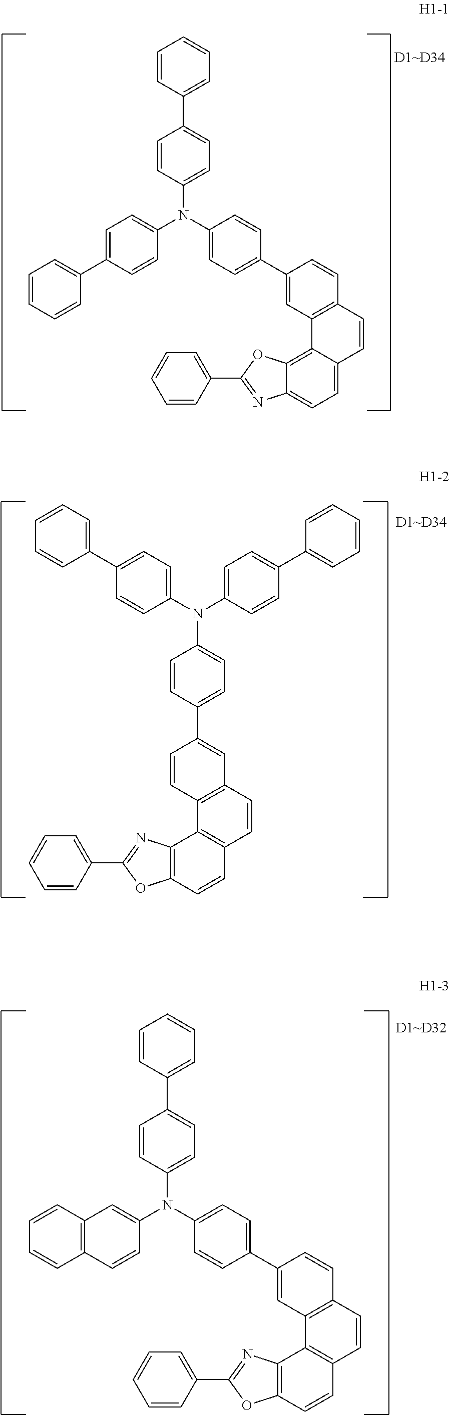

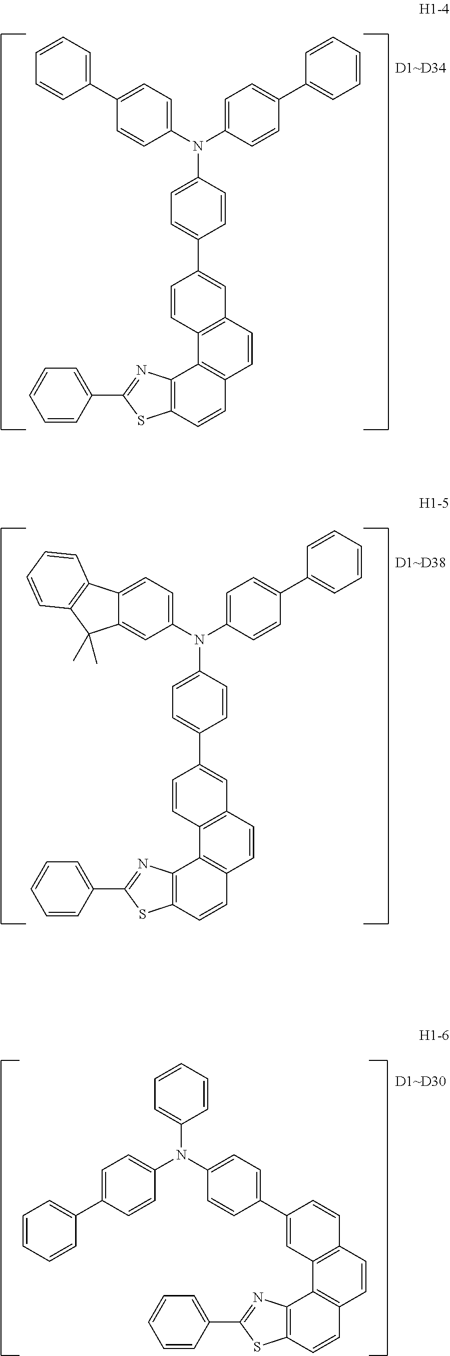

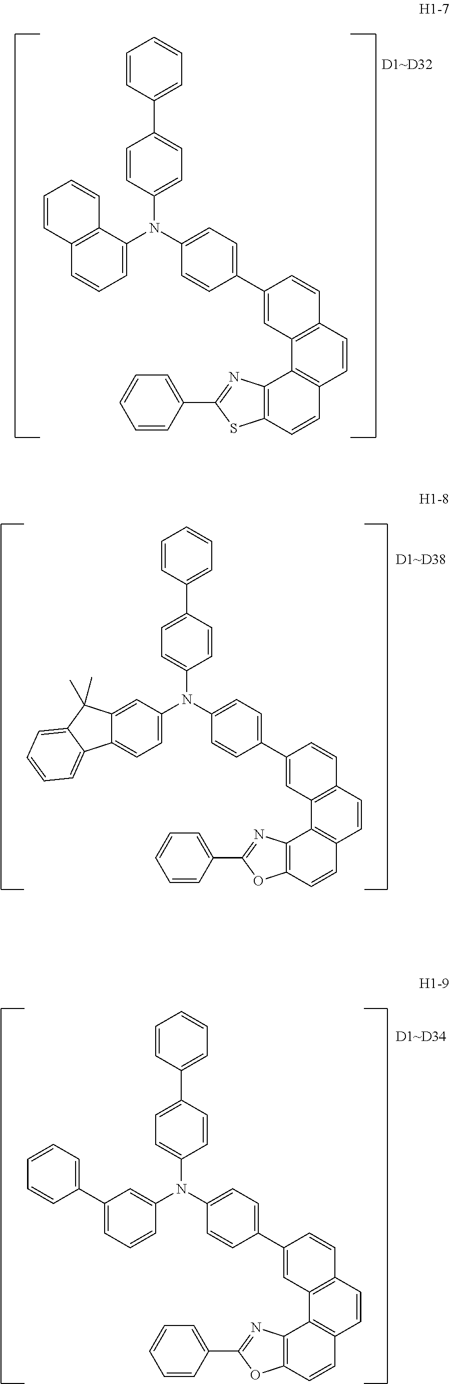

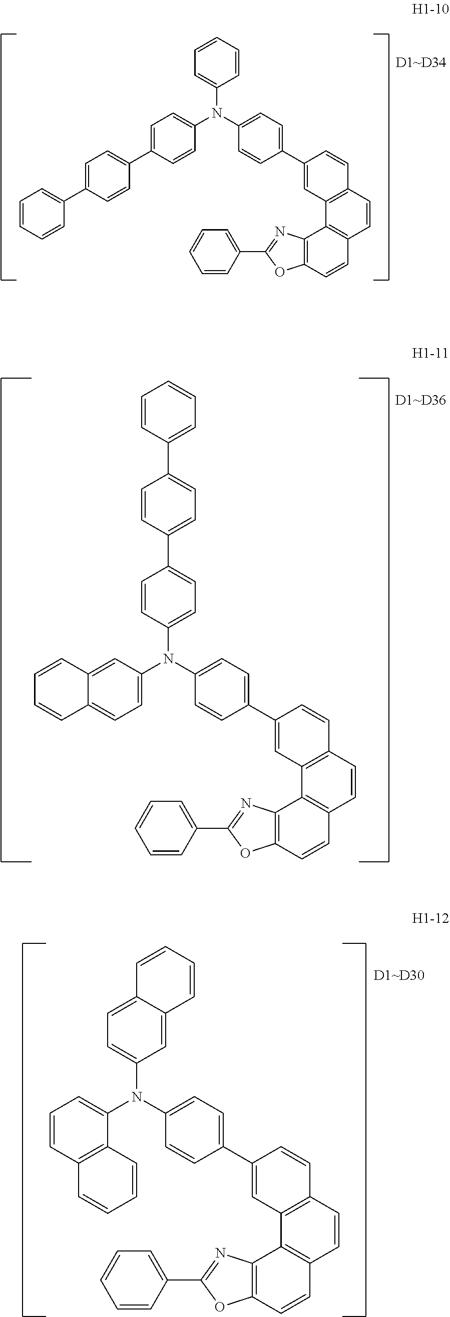

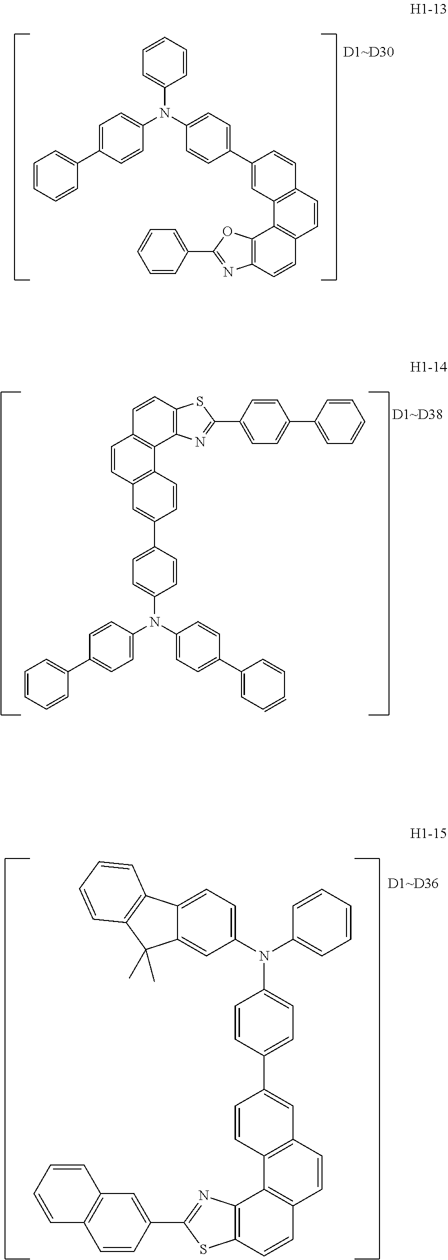

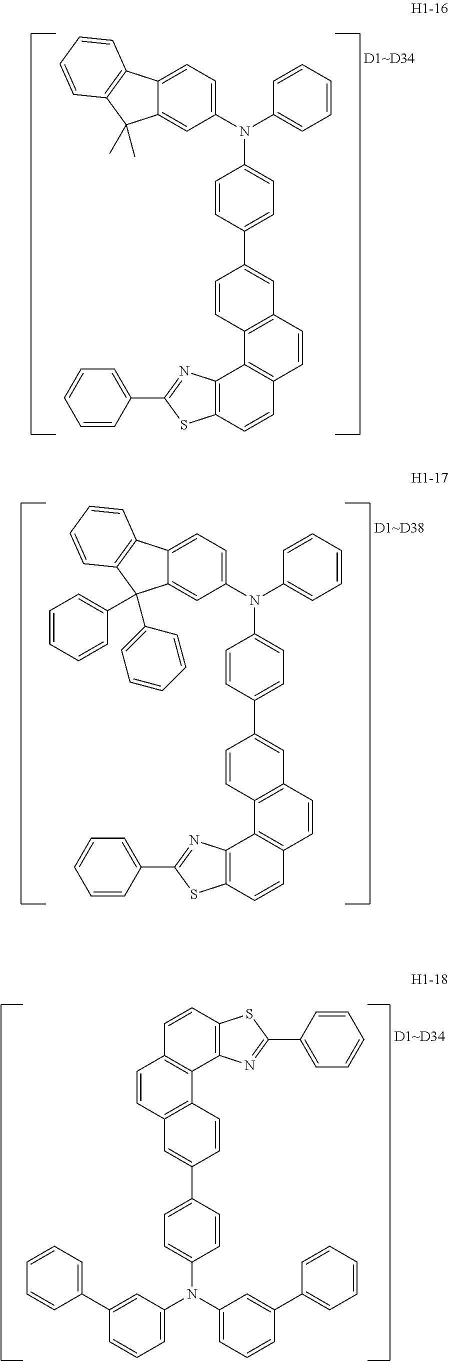

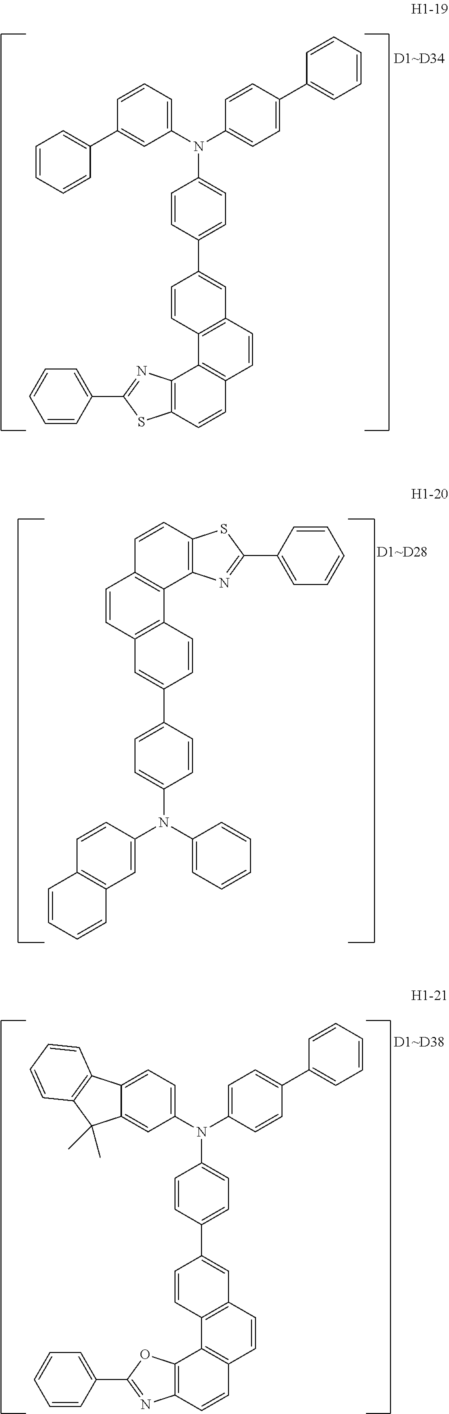

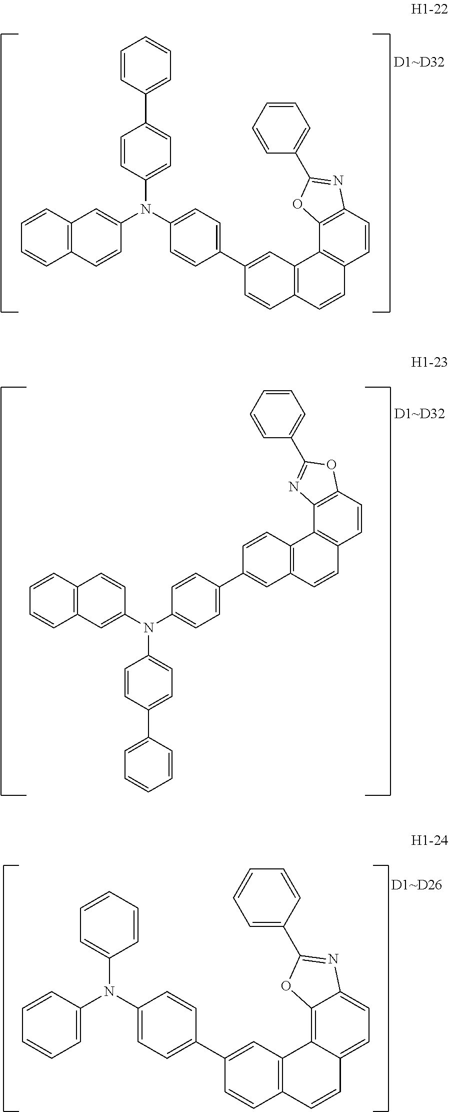

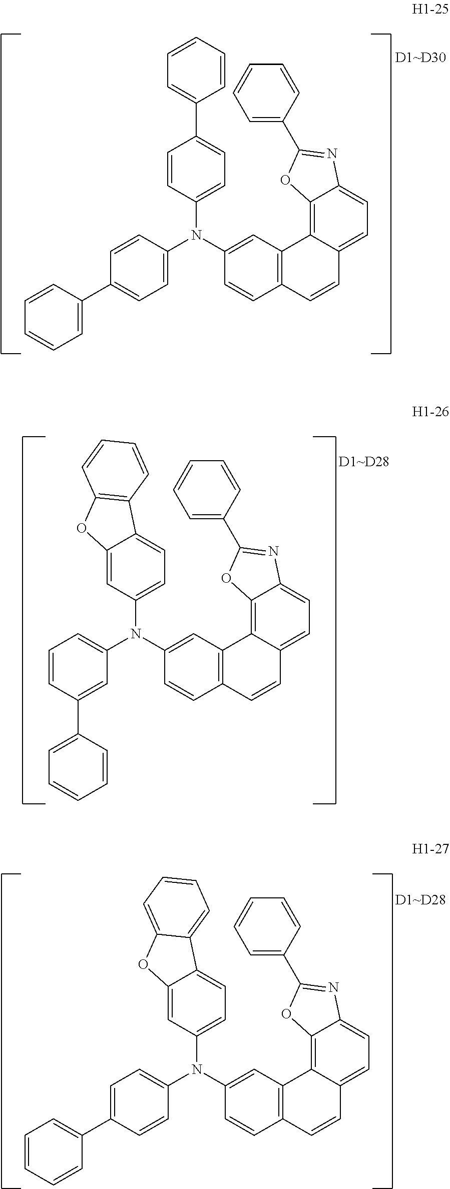

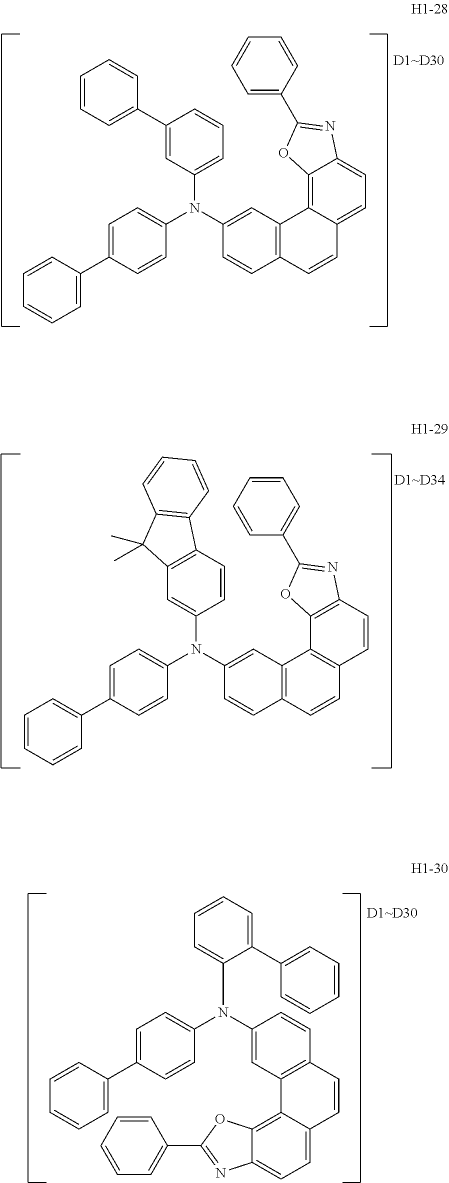

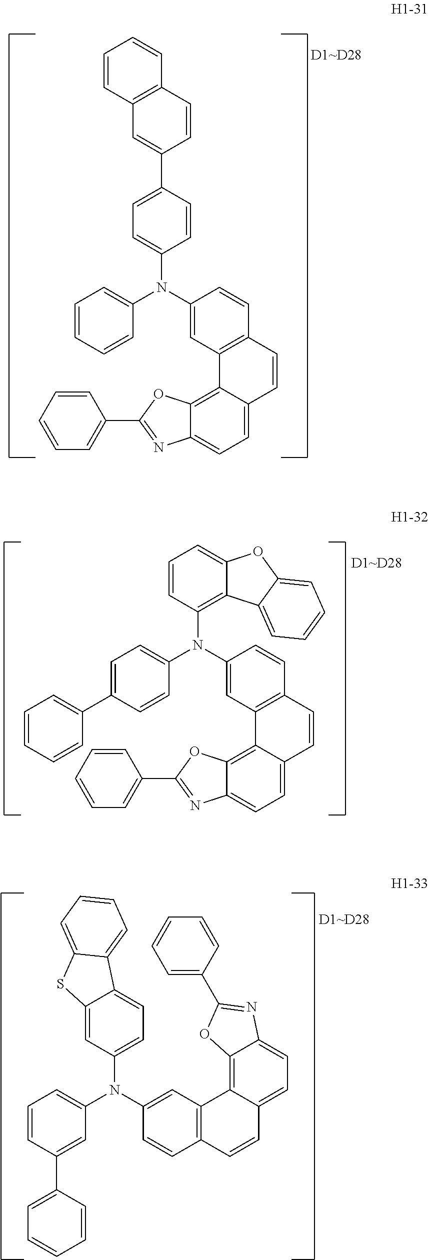

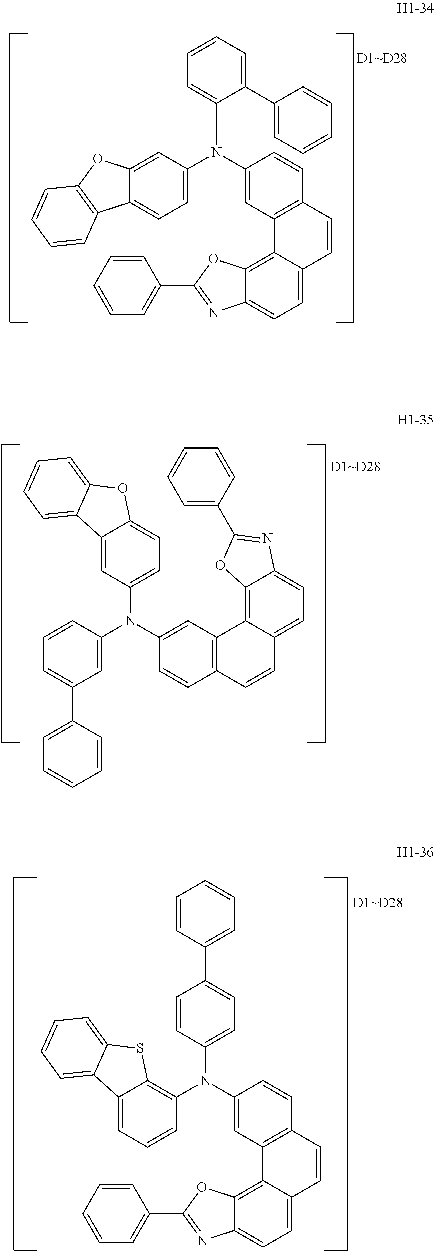

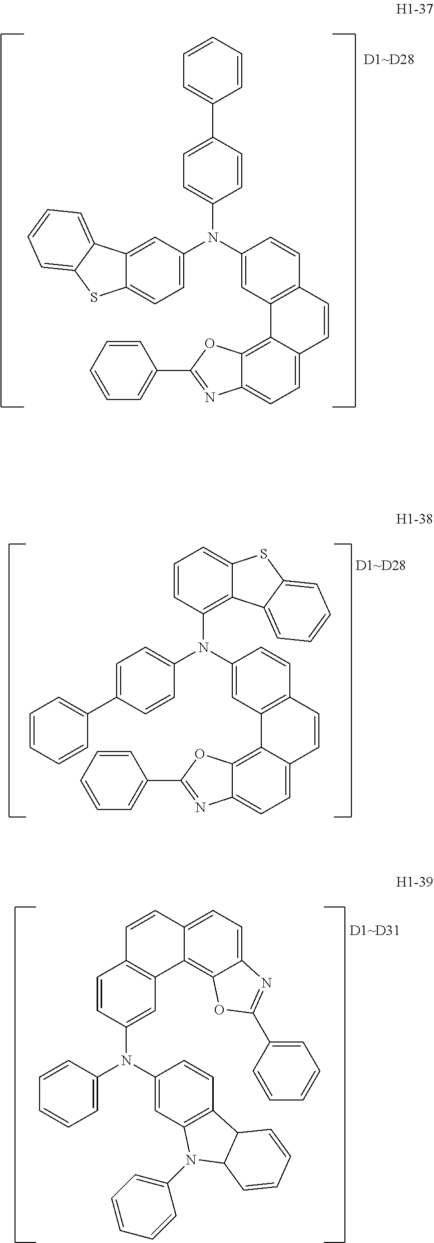

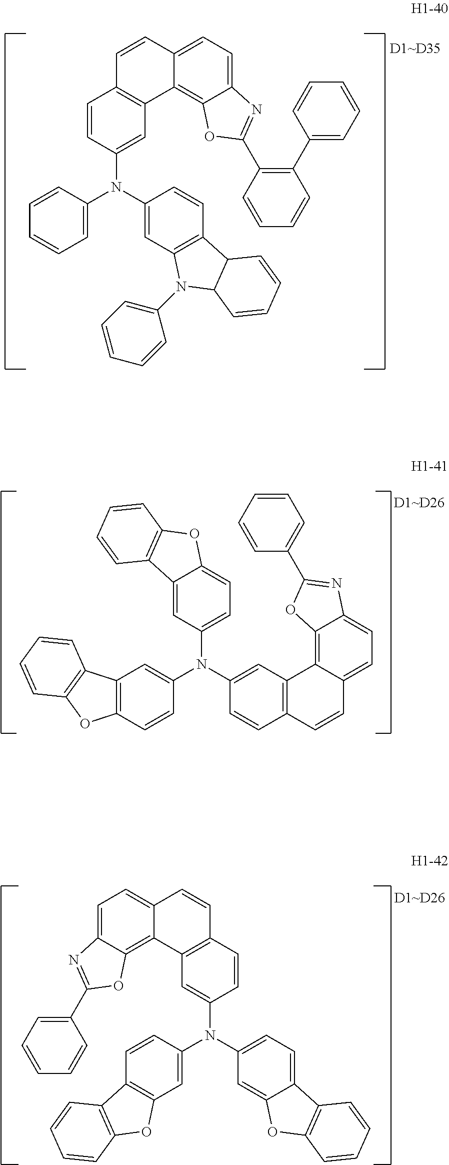

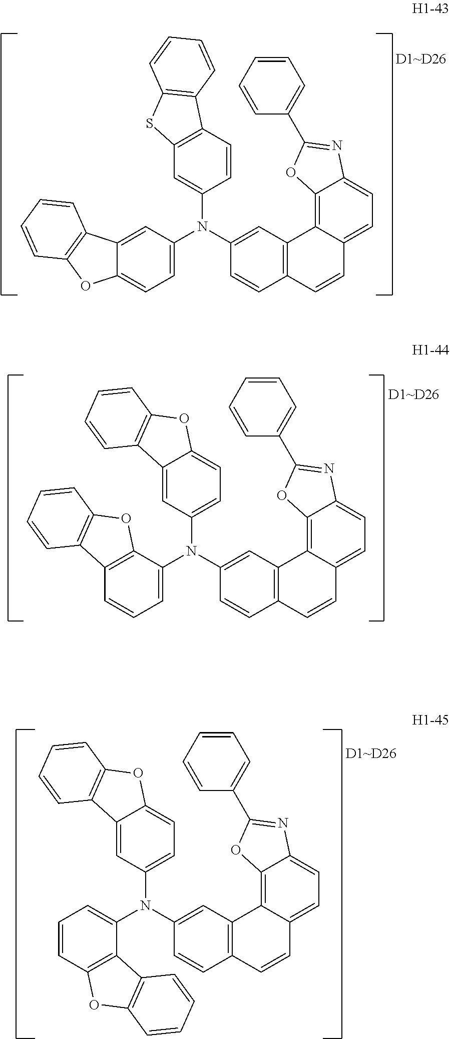

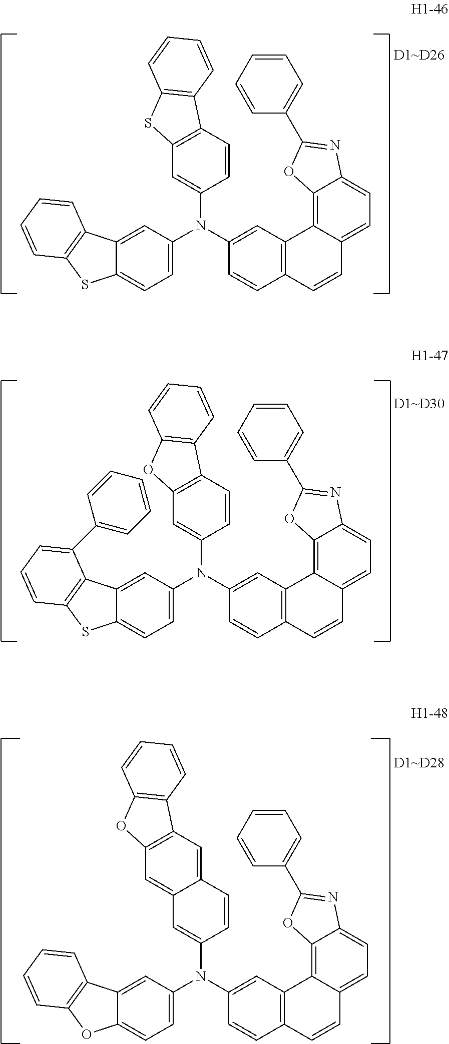

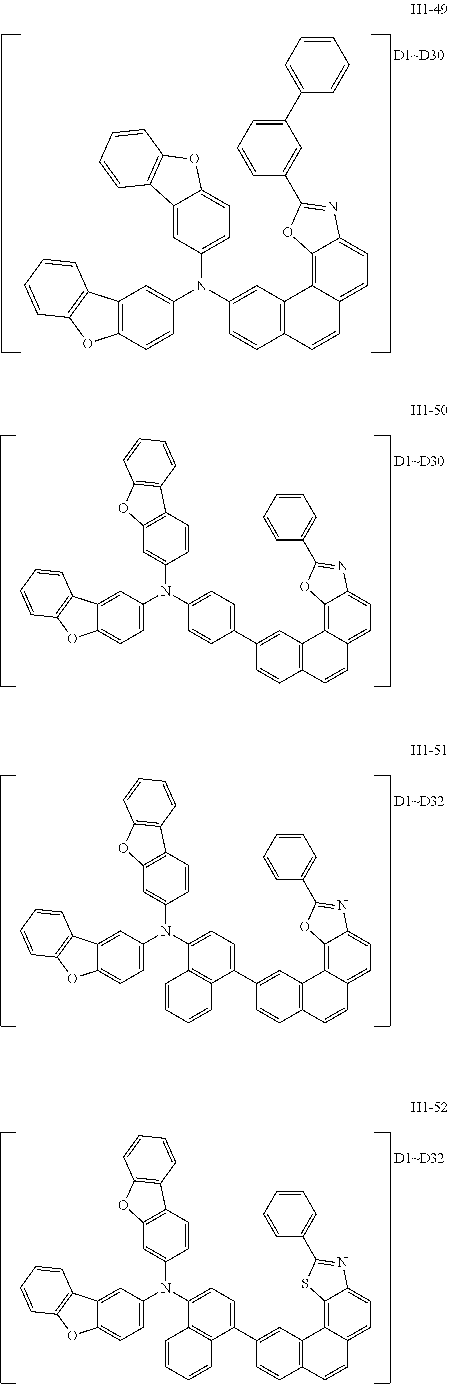

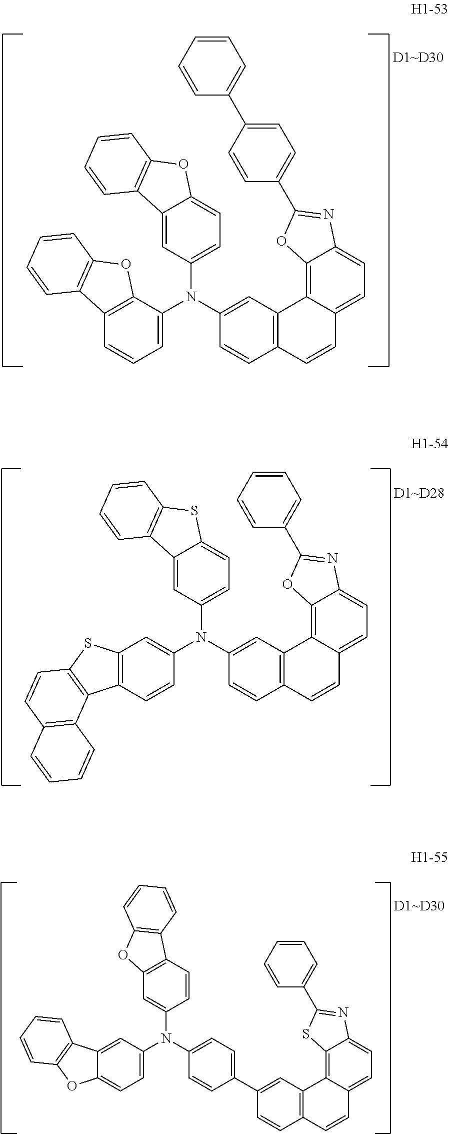

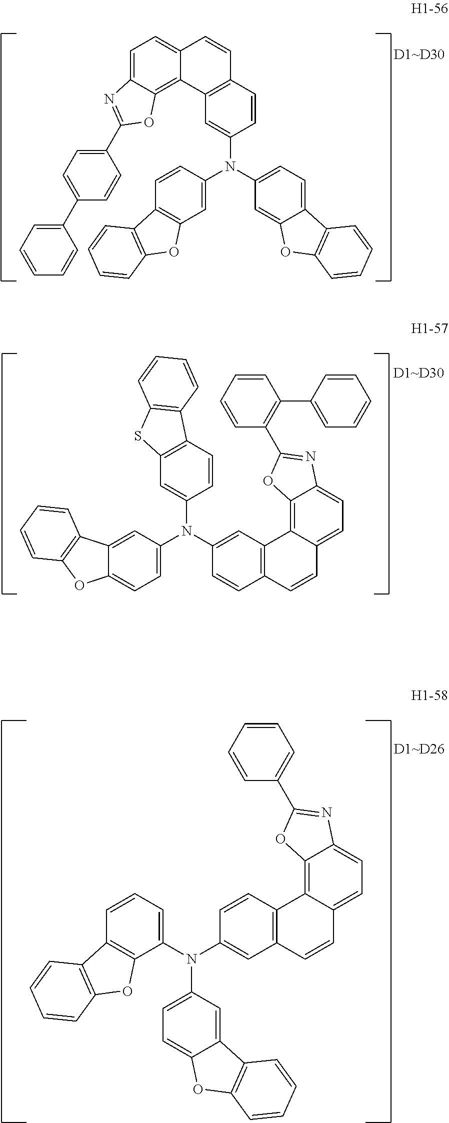

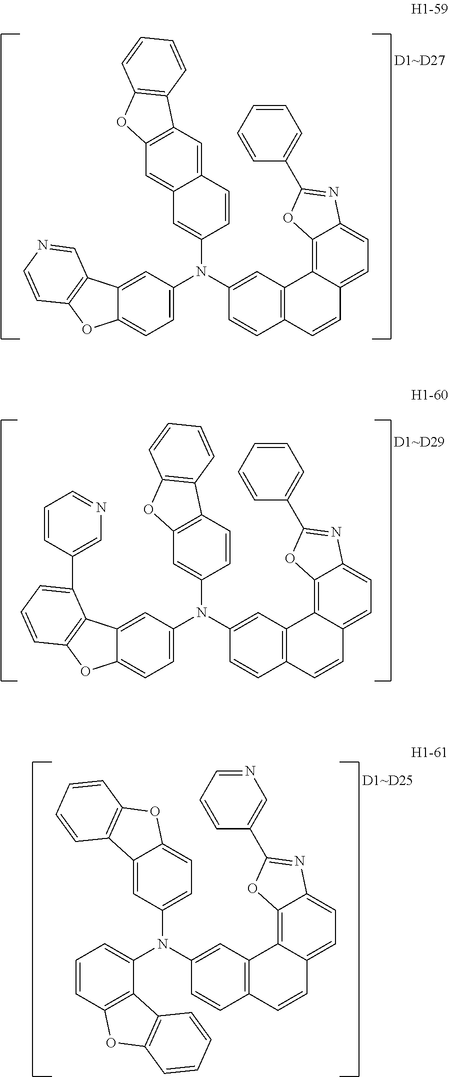

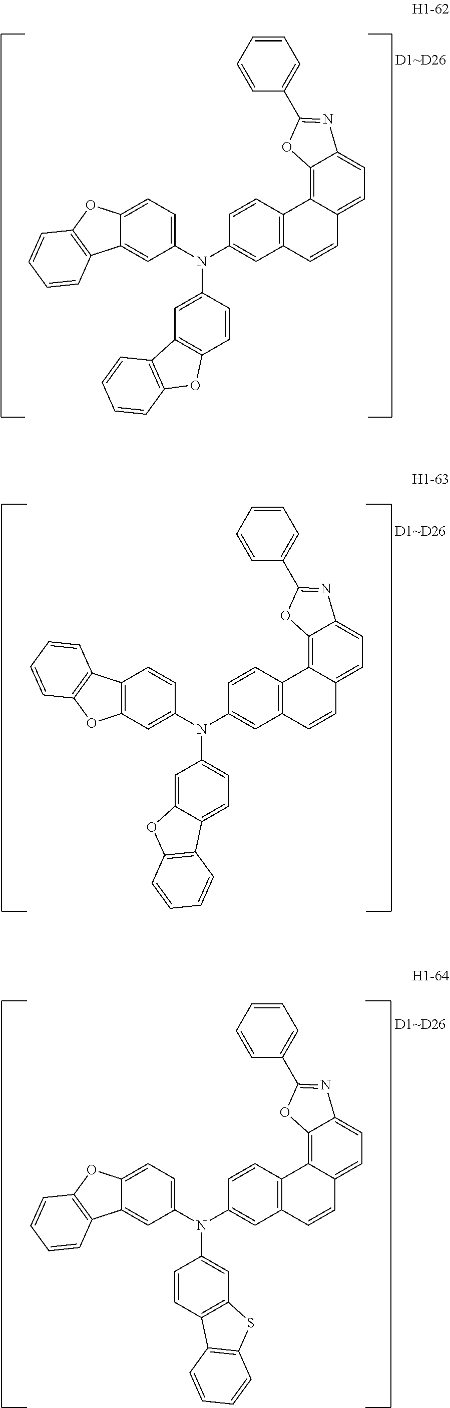

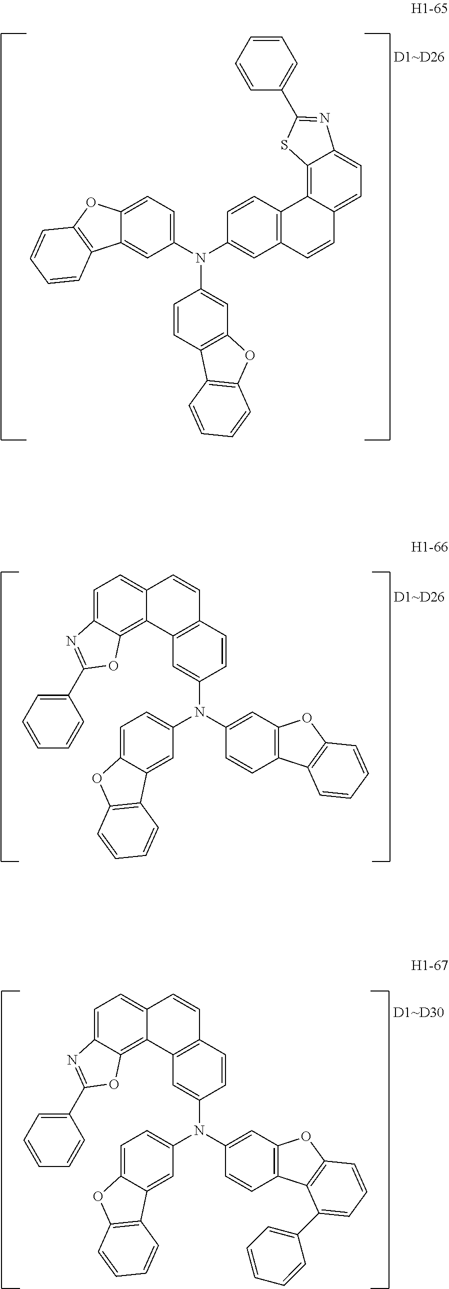

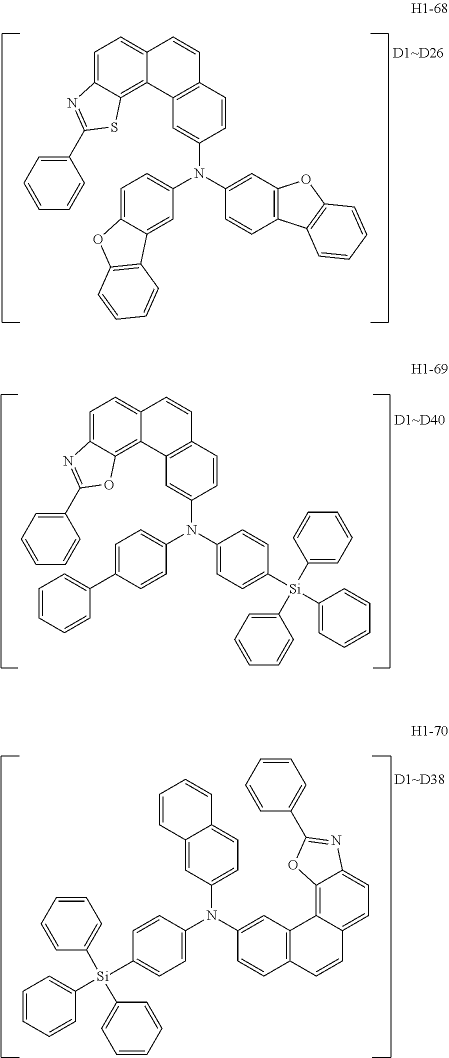

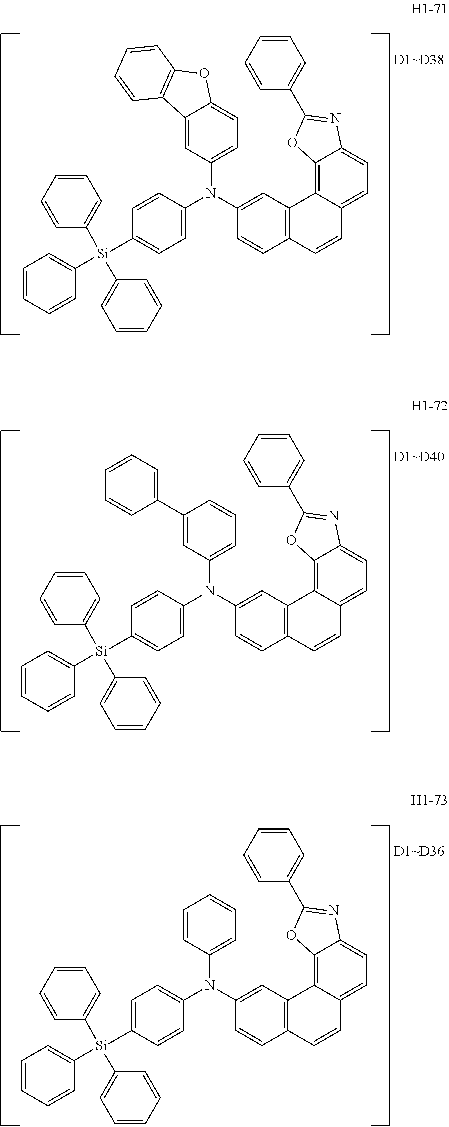

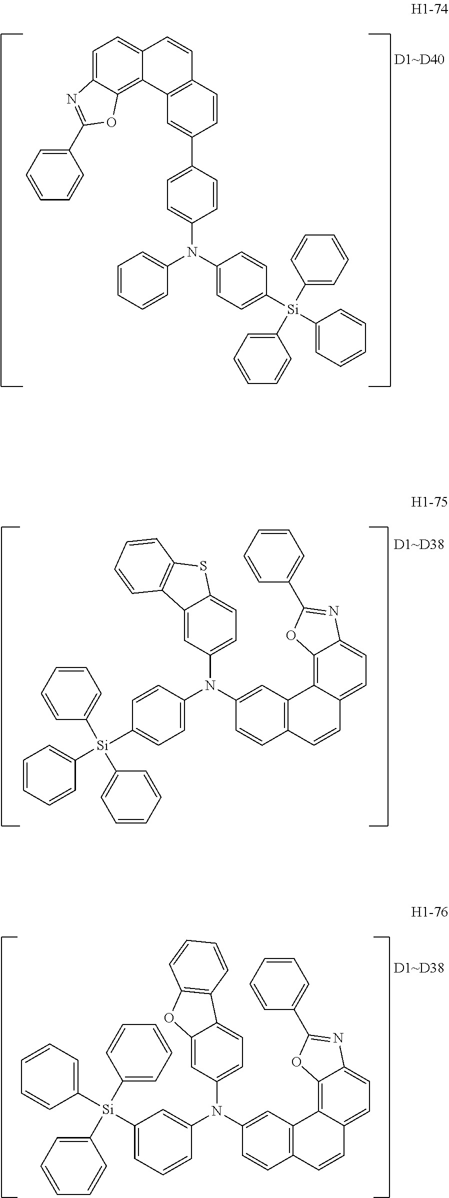

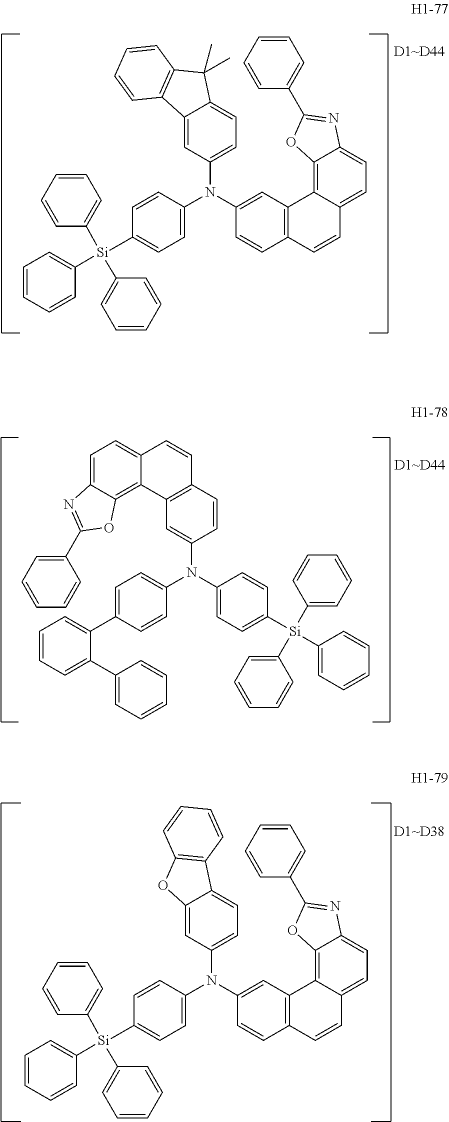

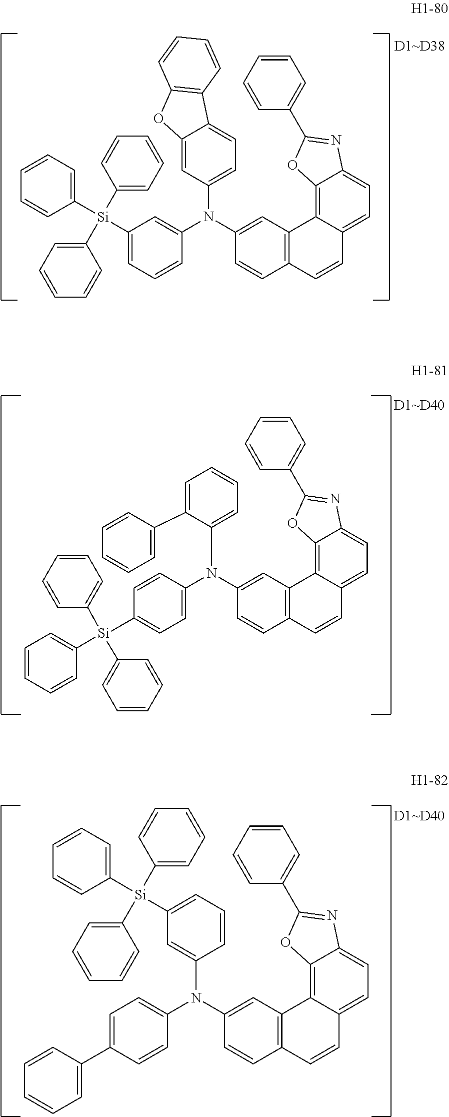

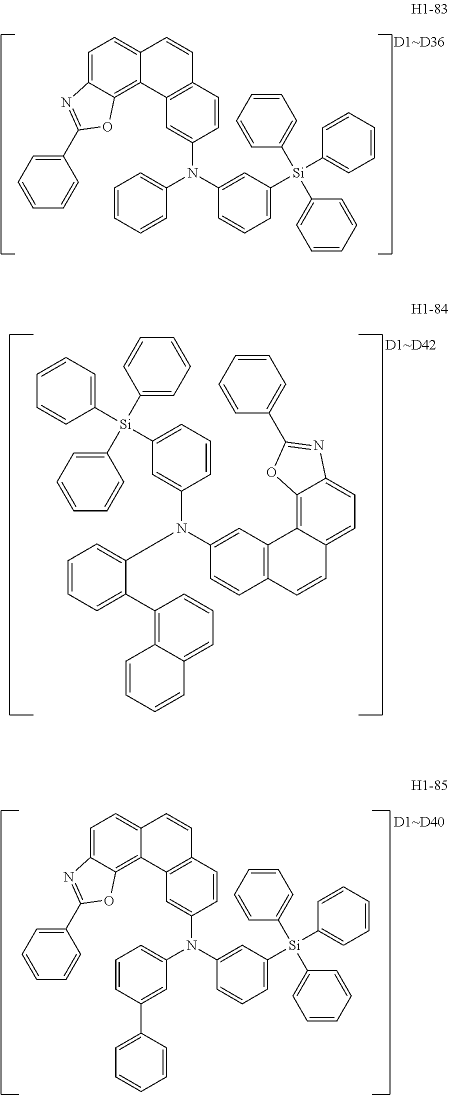

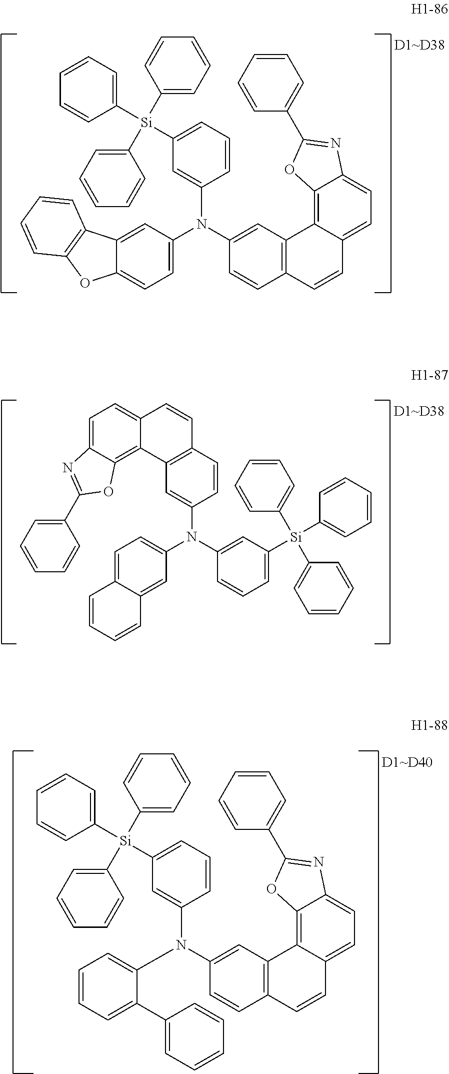

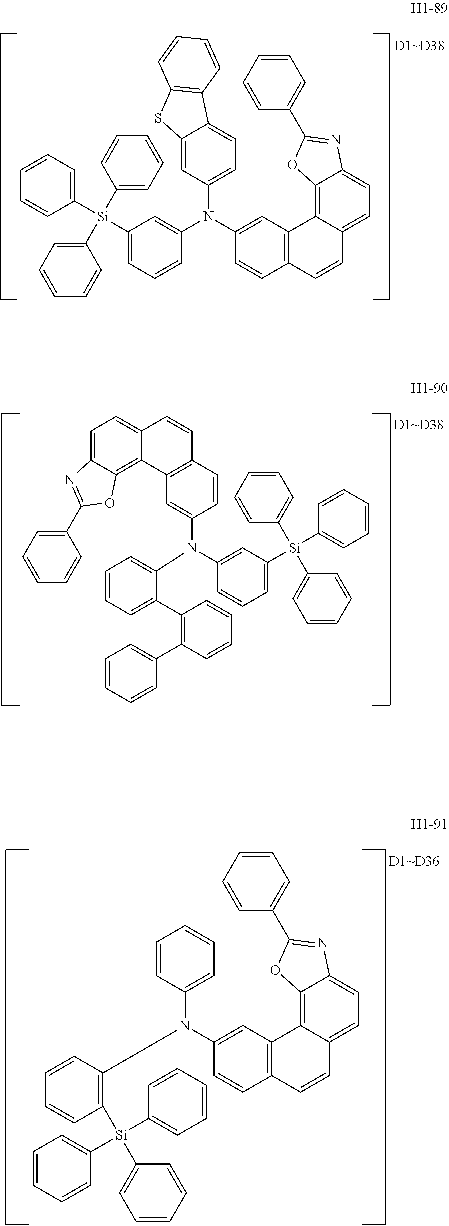

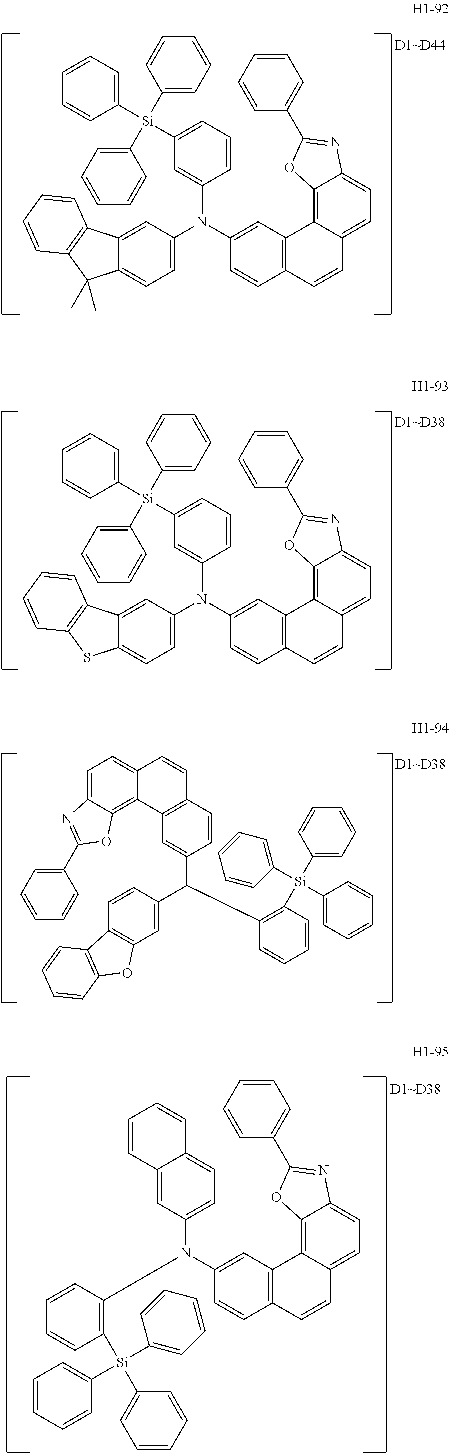

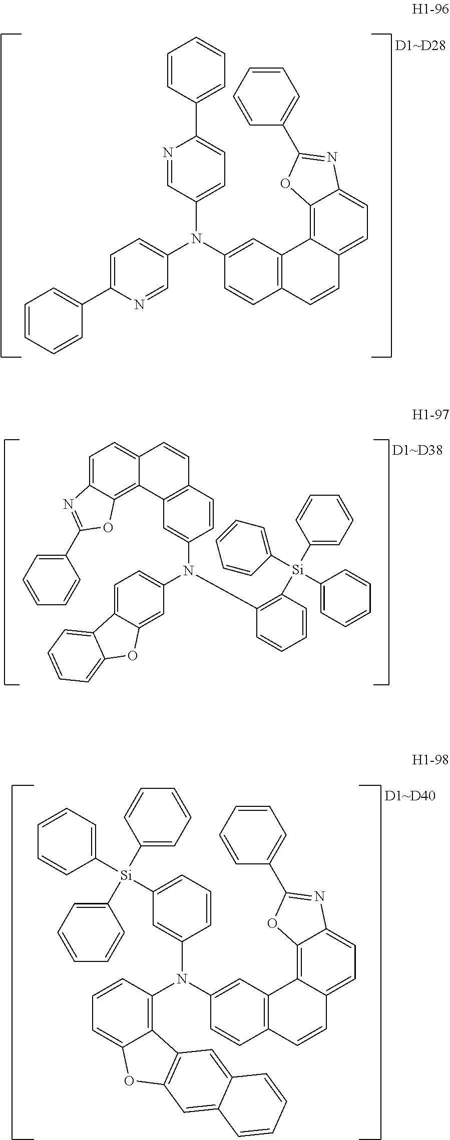

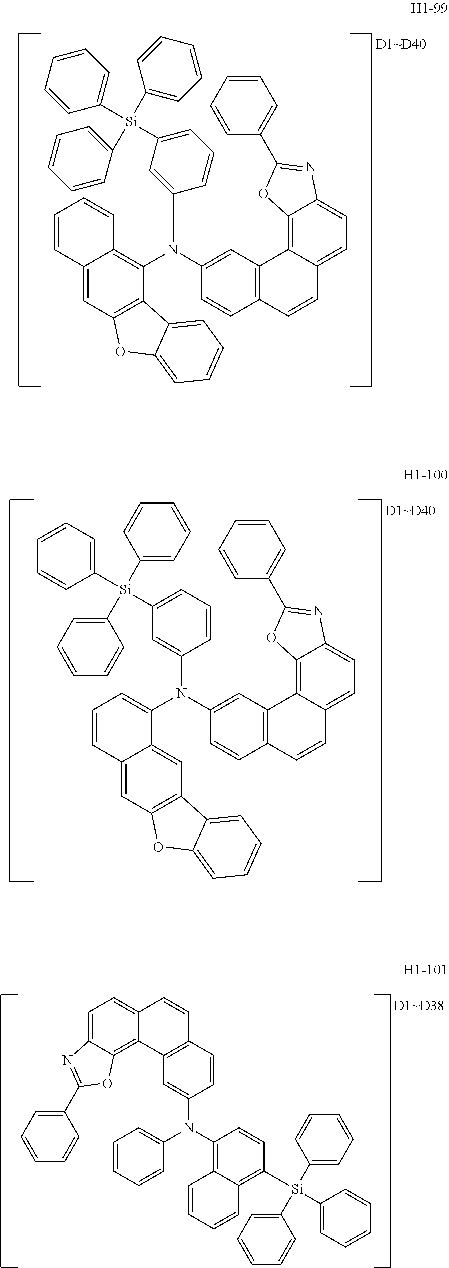

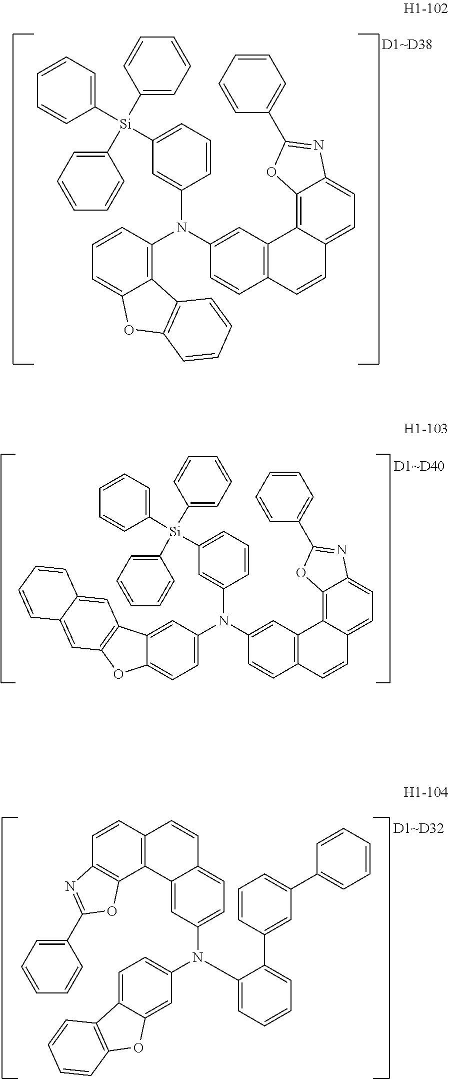

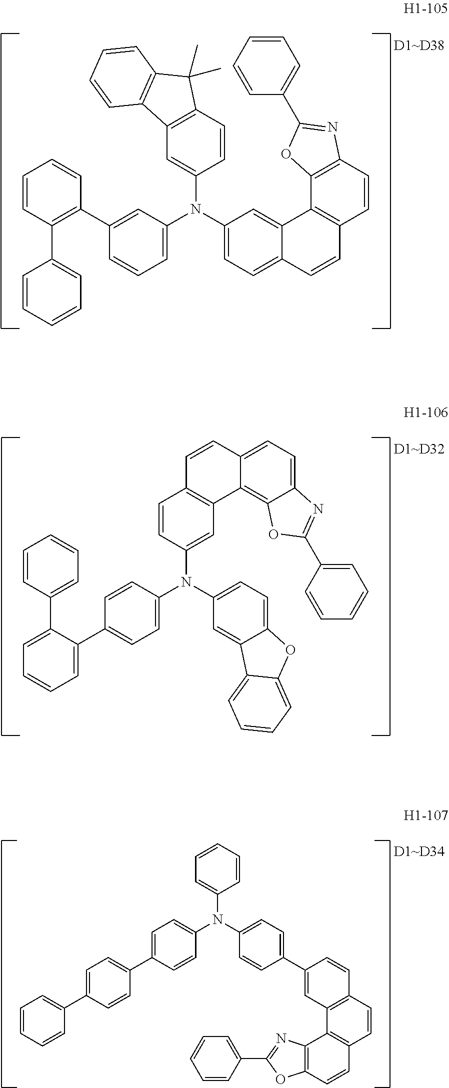

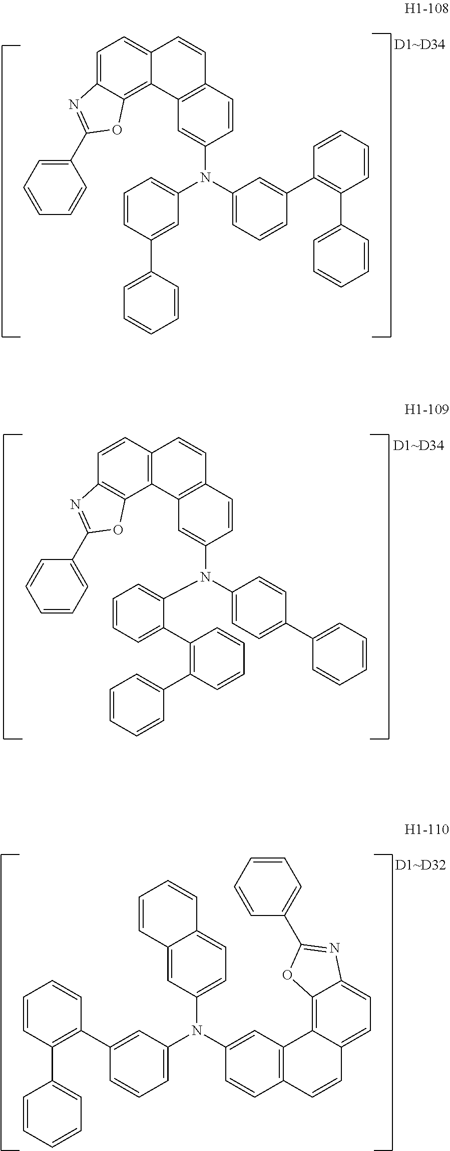

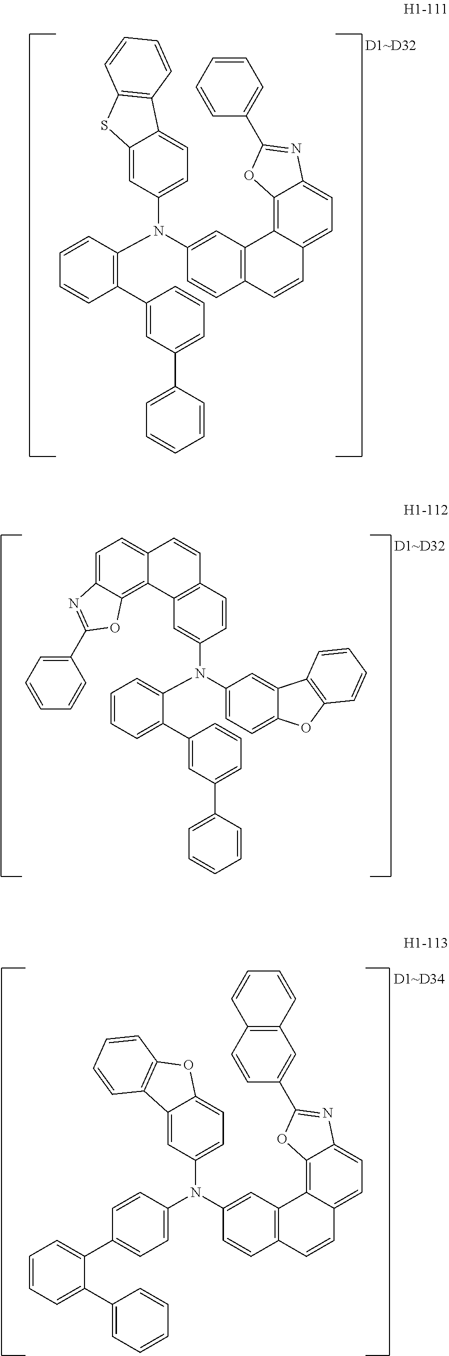

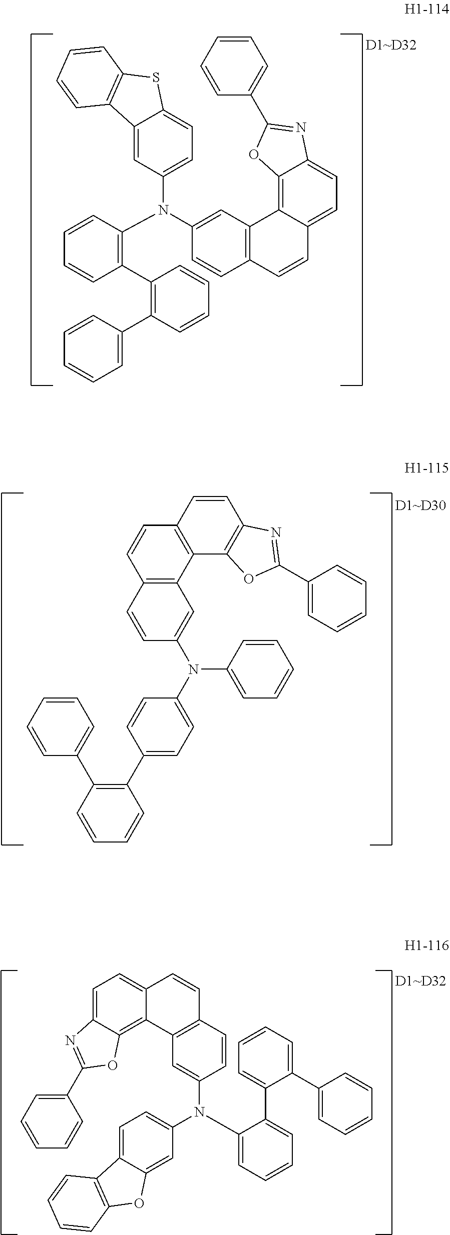

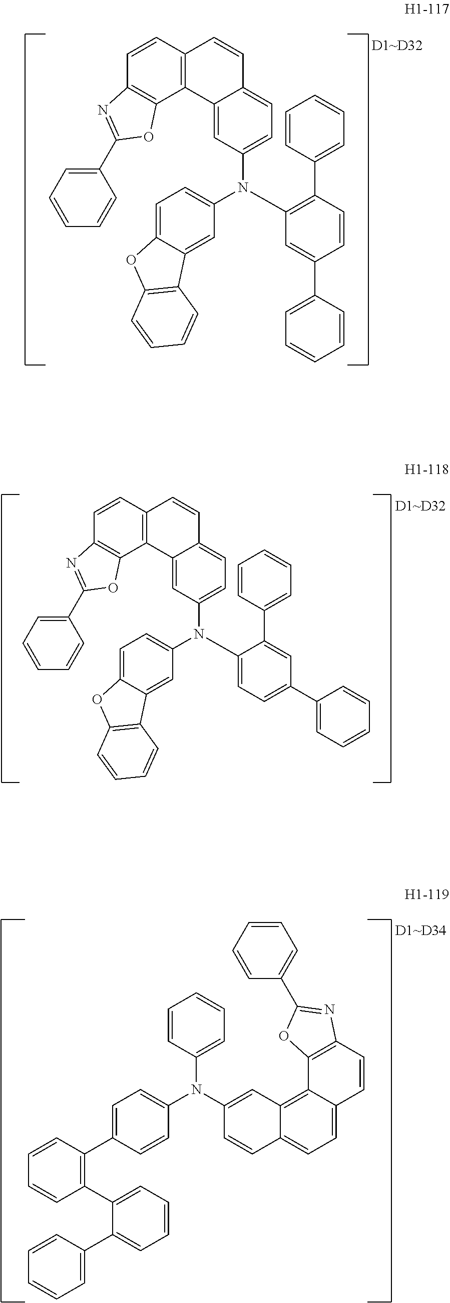

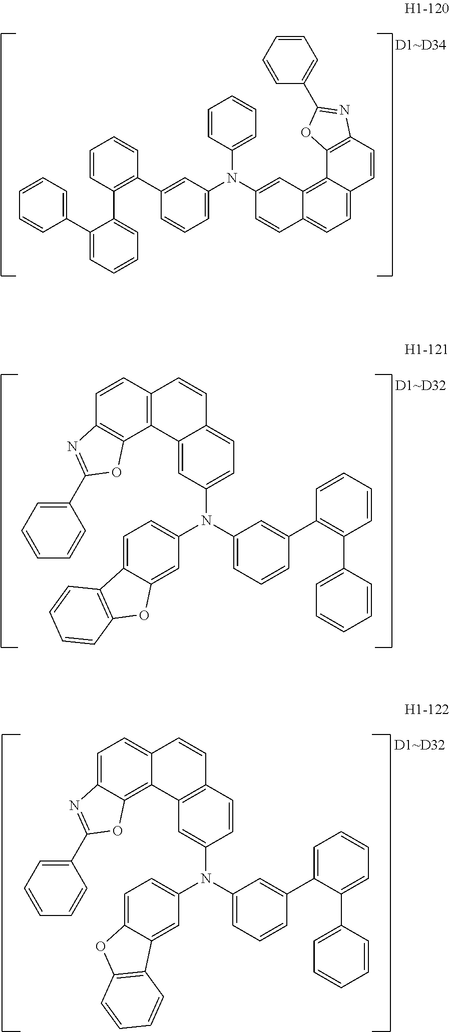

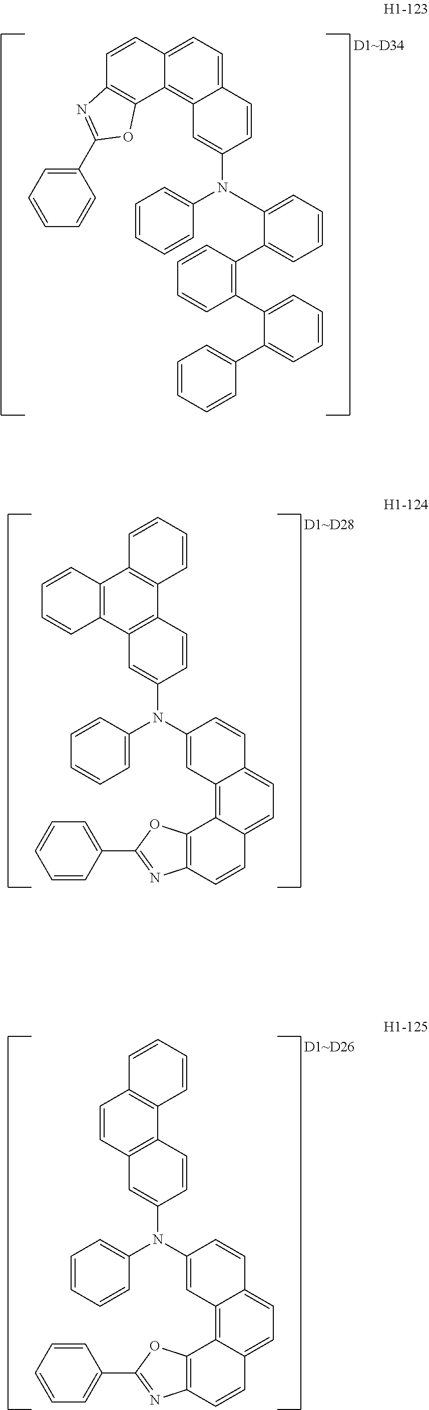

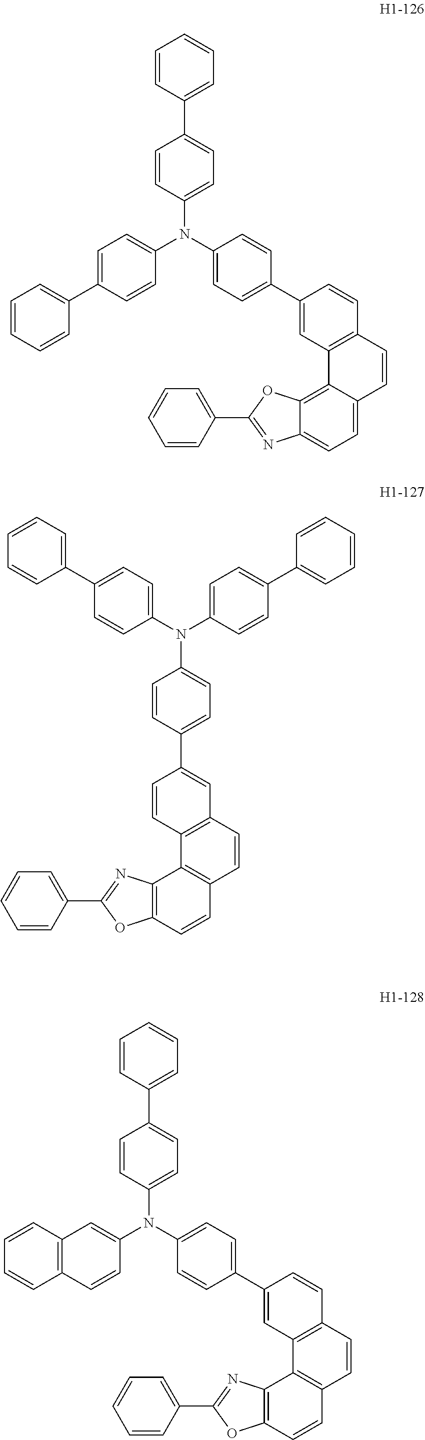 [SMILES: c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1]

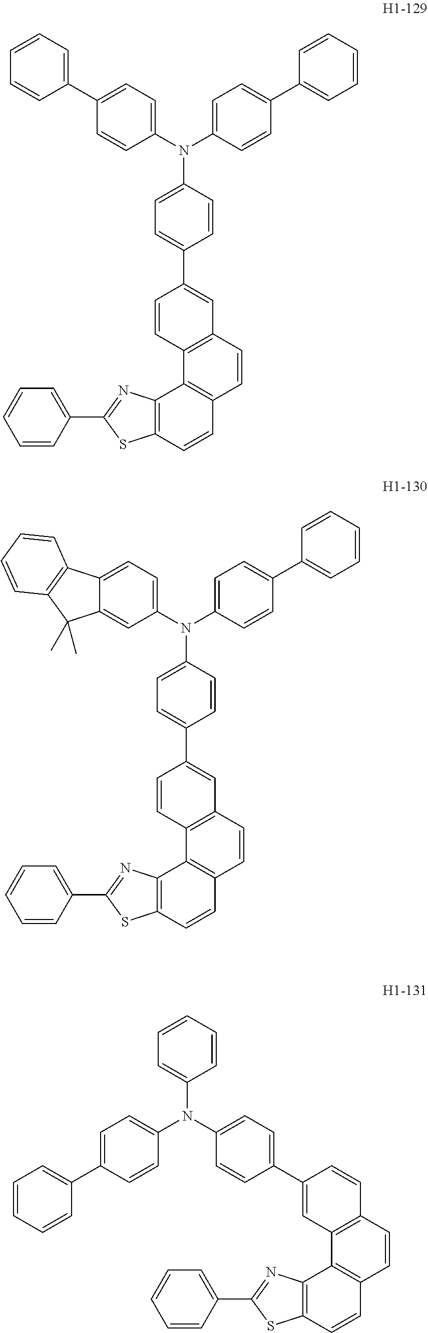 [SMILES: c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; c1ccc(-c2ccc(N(c3ccccc3)c3ccc(-c4ccc5ccc6ccc7sc(-c8ccccc8)nc7c6c5c4)cc3)cc2)cc1]

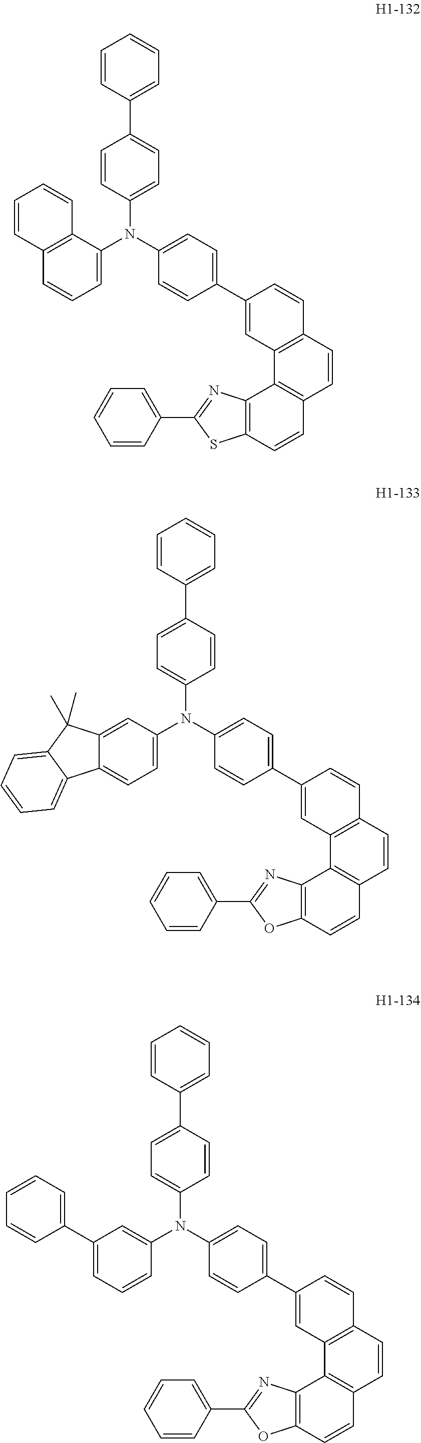 [SMILES: c1ccc(-c2ccc(N(c3ccc(-c4ccc5ccc6ccc7sc(-c8ccccc8)nc7c6c5c4)cc3)c3cccc4ccccc34)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1]

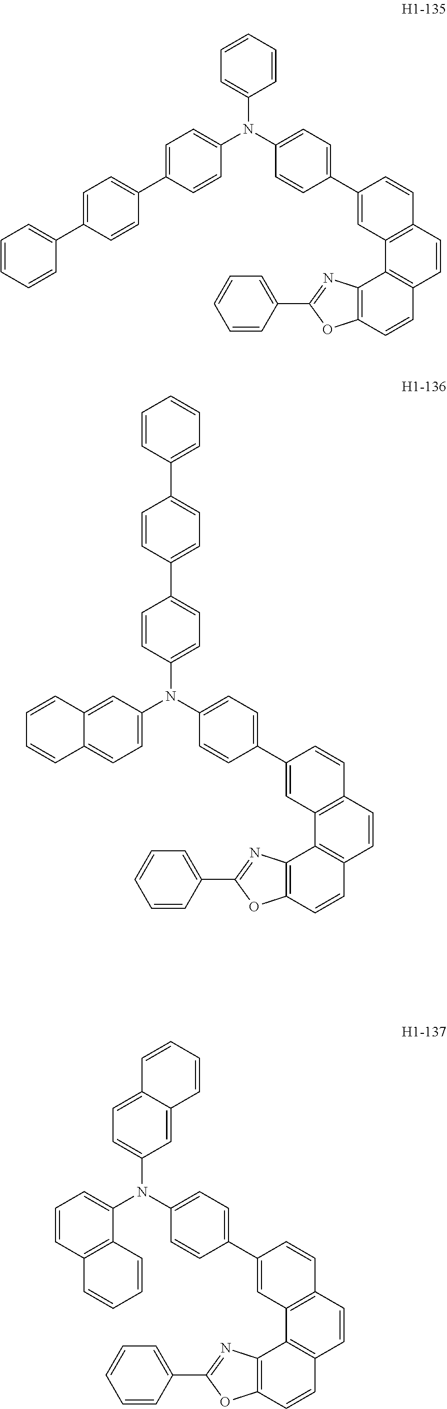 [SMILES: c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1]

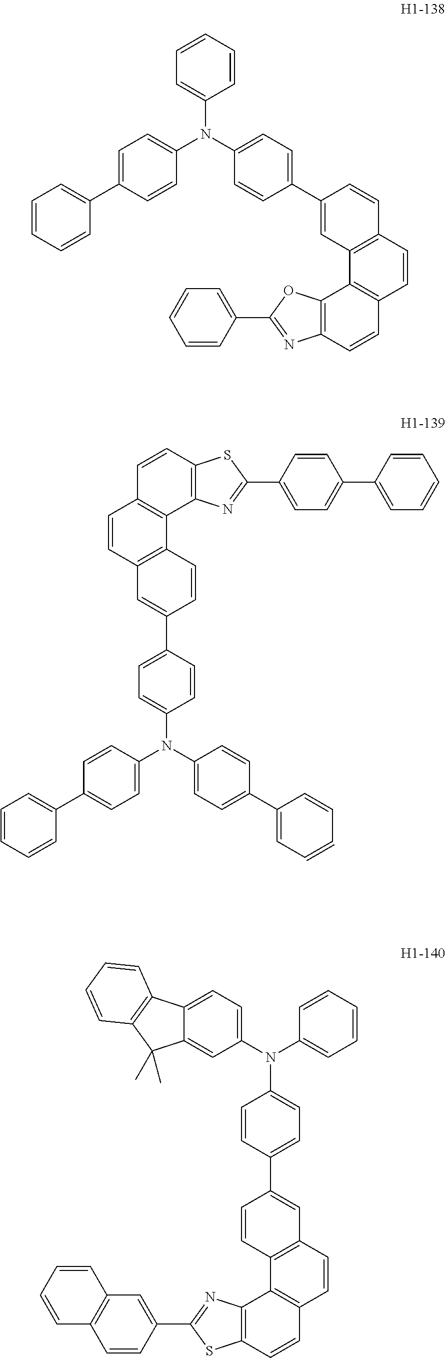 [SMILES: c1ccc(-c2ccc(N(c3ccccc3)c3ccc(-c4ccc5ccc6ccc7nc(-c8ccccc8)oc7c6c5c4)cc3)cc2)cc1 ; c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccccc3)c3ccc(-c4ccc5c(ccc6ccc7sc(-c8ccc9ccccc9c8)nc7c65)c4)cc3)cc21]

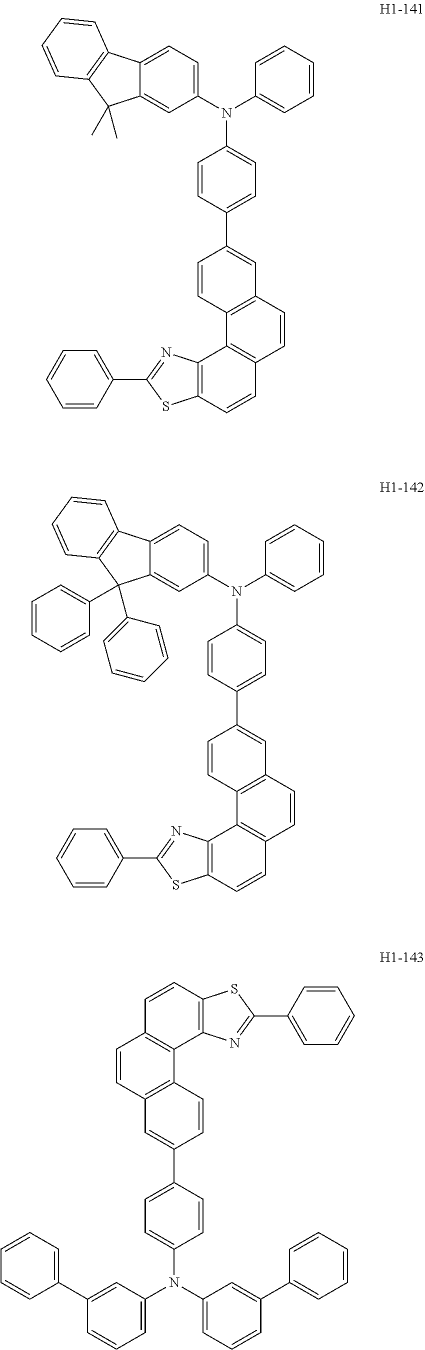 [SMILES: CC1(C)c2ccccc2-c2ccc(N(c3ccccc3)c3ccc(-c4ccc5c(ccc6ccc7sc(-c8ccccc8)nc7c65)c4)cc3)cc21 ; c1ccc(-c2nc3c(ccc4ccc5cc(-c6ccc(N(c7ccccc7)c7ccc8c(c7)C(c7ccccc7)(c7ccccc7)c7ccccc7-8)cc6)ccc5c43)s2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1]

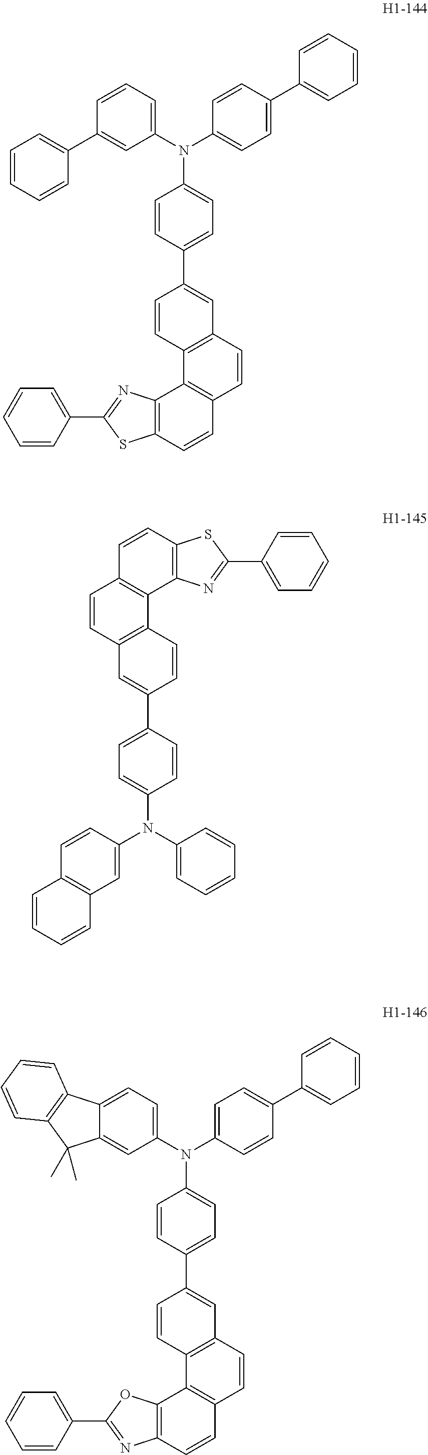 [SMILES: c1ccc(-c2ccc(N(c3ccc(-c4ccc5c(ccc6ccc7sc(-c8ccccc8)nc7c65)c4)cc3)c3cccc(-c4ccccc4)c3)cc2)cc1 ; c1ccc(-c2nc3c(ccc4ccc5cc(-c6ccc(N(c7ccccc7)c7ccc8ccccc8c7)cc6)ccc5c43)s2)cc1 ; CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21]

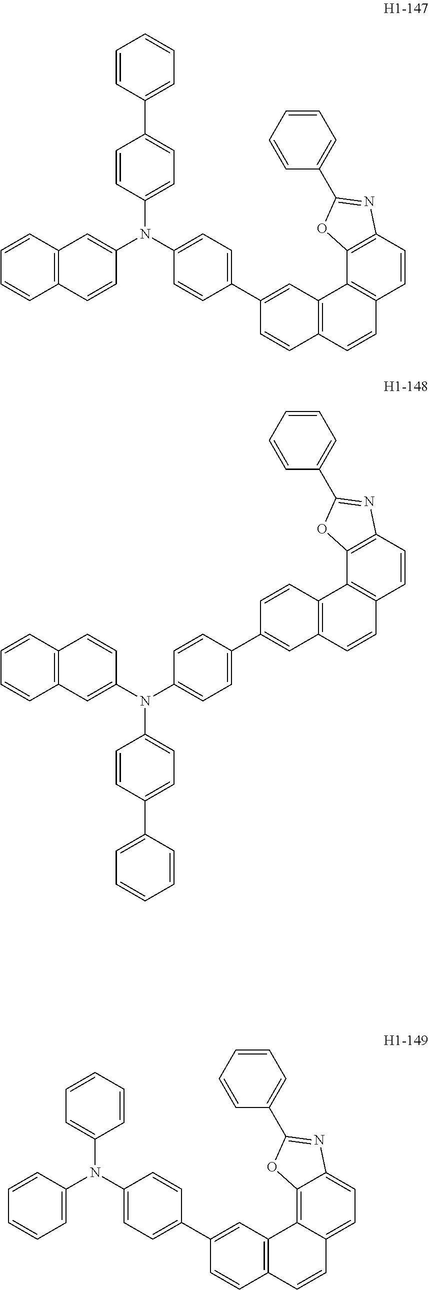 [SMILES: c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7nc(-c8ccccc8)oc7c65)c4)cc3)c3ccc4ccccc4c3)cc2)cc1 ; c1ccc(-c2nc3ccc4ccc5ccc(-c6ccc(N(c7ccccc7)c7ccccc7)cc6)cc5c4c3o2)cc1]

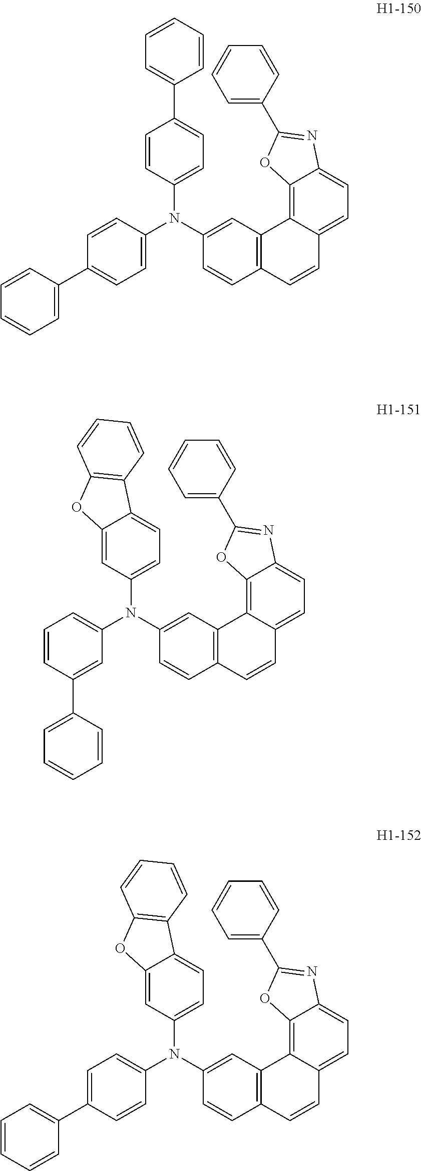 [SMILES: c1ccc(-c2ccc(N(c3ccc(-c4ccccc4)cc3)c3ccc4ccc5ccc6nc(-c7ccccc7)oc6c5c4c3)cc2)cc1 ; c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3ccc4c(c3)oc3ccccc34)c3ccc4ccc5ccc6nc(-c7ccccc7)oc6c5c4c3)cc2)cc1]

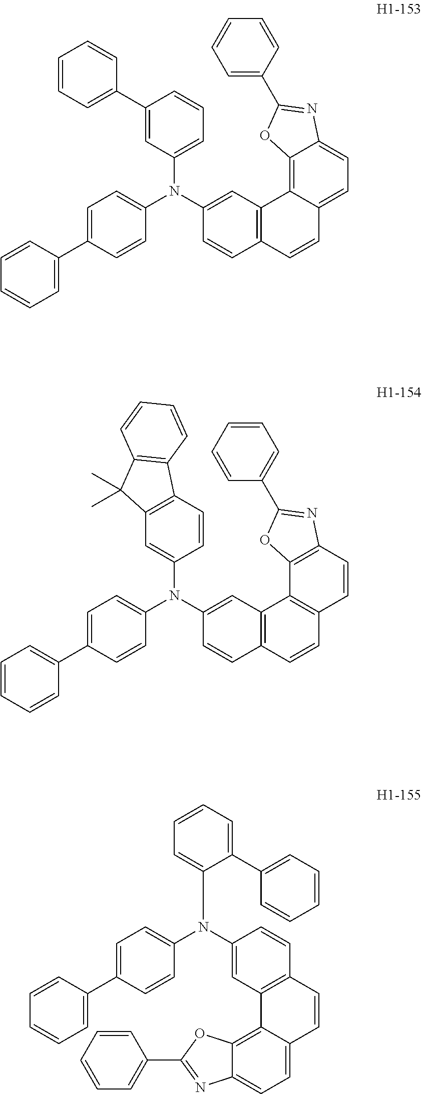 [SMILES: c1ccc(-c2ccc(N(c3cccc(-c4ccccc4)c3)c3ccc4ccc5ccc6nc(-c7ccccc7)oc6c5c4c3)cc2)cc1 ; CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc4ccc5ccc6nc(-c7ccccc7)oc6c5c4c3)c3ccccc3-c3ccccc3)cc2)cc1]

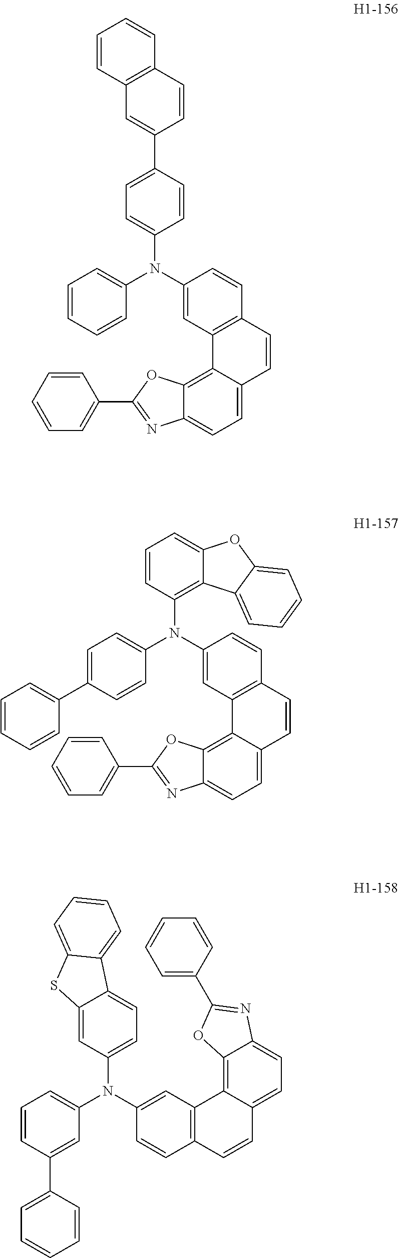 [SMILES: c1ccc(-c2nc3ccc4ccc5ccc(N(c6ccccc6)c6ccc(-c7ccc8ccccc8c7)cc6)cc5c4c3o2)cc1 ; c1ccc(-c2ccc(N(c3ccc4ccc5ccc6nc(-c7ccccc7)oc6c5c4c3)c3cccc4oc5ccccc5c34)cc2)cc1 ; c1ccc(-c2cccc(N(c3ccc4c(c3)sc3ccccc34)c3ccc4ccc5ccc6nc(-c7ccccc7)oc6c5c4c3)c2)cc1]

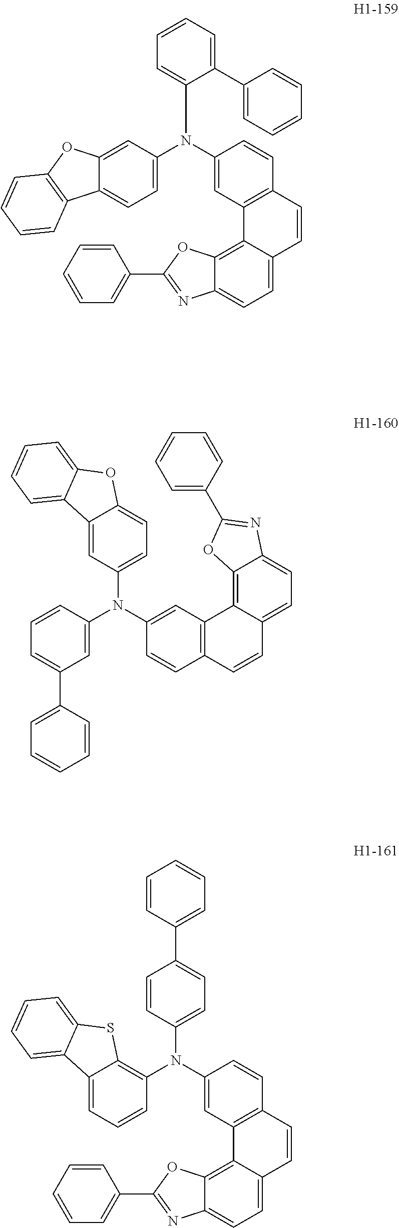 [SMILES: c1ccc(-c2nc3ccc4ccc5ccc(N(c6ccc7c(c6)oc6ccccc67)c6ccccc6-c6ccccc6)cc5c4c3o2)cc1 ; c1ccc(-c2cccc(N(c3ccc4oc5ccccc5c4c3)c3ccc4ccc5ccc6nc(-c7ccccc7)oc6c5c4c3)c2)cc1 ; c1ccc(-c2ccc(N(c3ccc4ccc5ccc6nc(-c7ccccc7)oc6c5c4c3)c3cccc4c3sc3ccccc34)cc2)cc1]

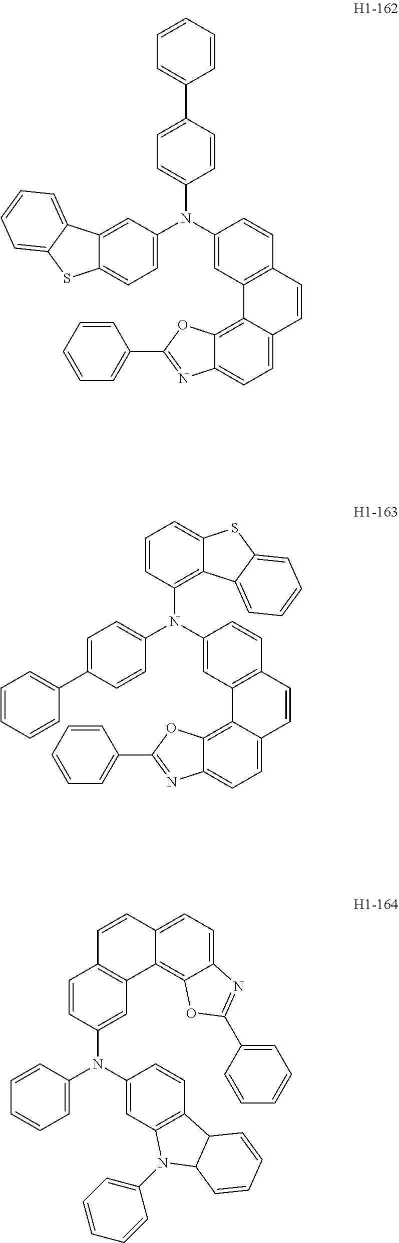 [SMILES: c1ccc(-c2ccc(N(c3ccc4sc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4sc5ccccc5c34)cc2)cc1 ; C1=CC2c3ccc(N(c4ccccc4)c4ccc5ccc6ccc7nc(-c8ccccc8)oc7c6c5c4)cc3N(c3ccccc3)C2C=C1]

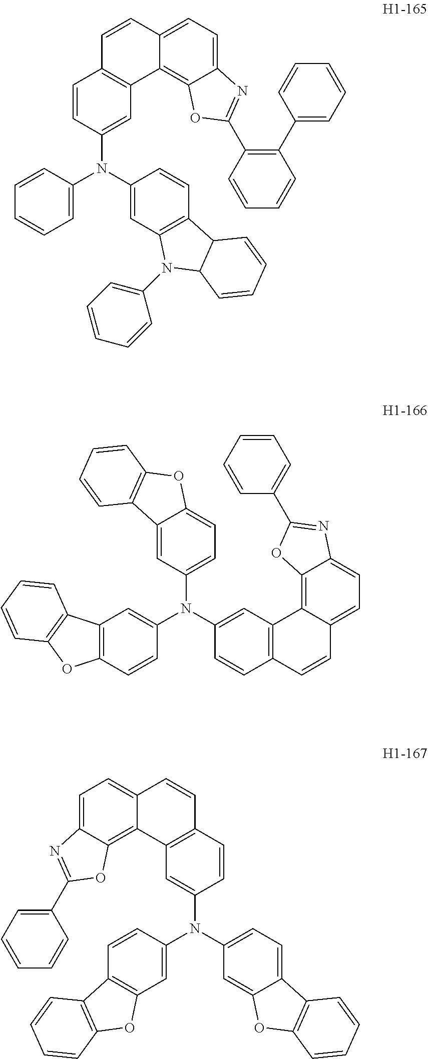 [SMILES: C1=CC2c3ccc(N(c4ccccc4)c4ccc5ccc6ccc7nc(-c8ccccc8-c8ccccc8)oc7c6c5c4)cc3N(c3ccccc3)C2C=C1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7c(c6)oc6ccccc67)cc5c4c3o2)cc1]

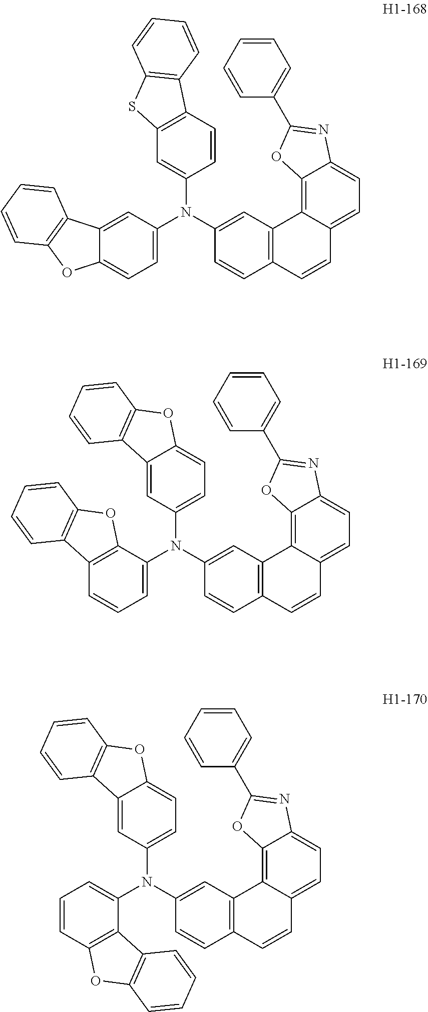 [SMILES: c1ccc(-c2nc3ccc4ccc5ccc(N(c6ccc7c(c6)sc6ccccc67)c6ccc7oc8ccccc8c7c6)cc5c4c3o2)cc1 ; c1ccc(-c2nc3ccc4ccc5ccc(N(c6ccc7oc8ccccc8c7c6)c6cccc7c6oc6ccccc67)cc5c4c3o2)cc1 ; c1ccc(-c2nc3ccc4ccc5ccc(N(c6ccc7oc8ccccc8c7c6)c6cccc7oc8ccccc8c67)cc5c4c3o2)cc1]

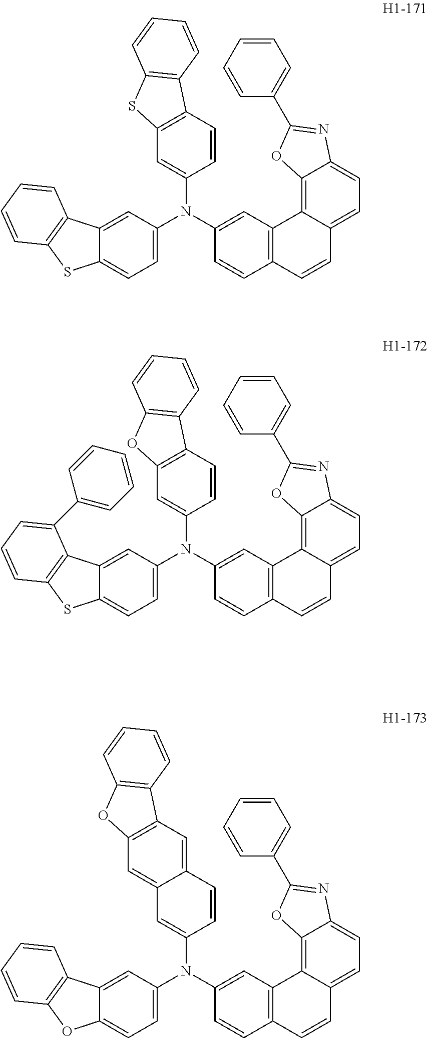 [SMILES: c1ccc(-c2nc3ccc4ccc5ccc(N(c6ccc7c(c6)sc6ccccc67)c6ccc7sc8ccccc8c7c6)cc5c4c3o2)cc1 ; c1ccc(-c2nc3ccc4ccc5ccc(N(c6ccc7c(c6)oc6ccccc67)c6ccc7sc8cccc(-c9ccccc9)c8c7c6)cc5c4c3o2)cc1 ; c1ccc(-c2nc3ccc4ccc5ccc(N(c6ccc7cc8c(cc7c6)oc6ccccc68)c6ccc7oc8ccccc8c7c6)cc5c4c3o2)cc1]

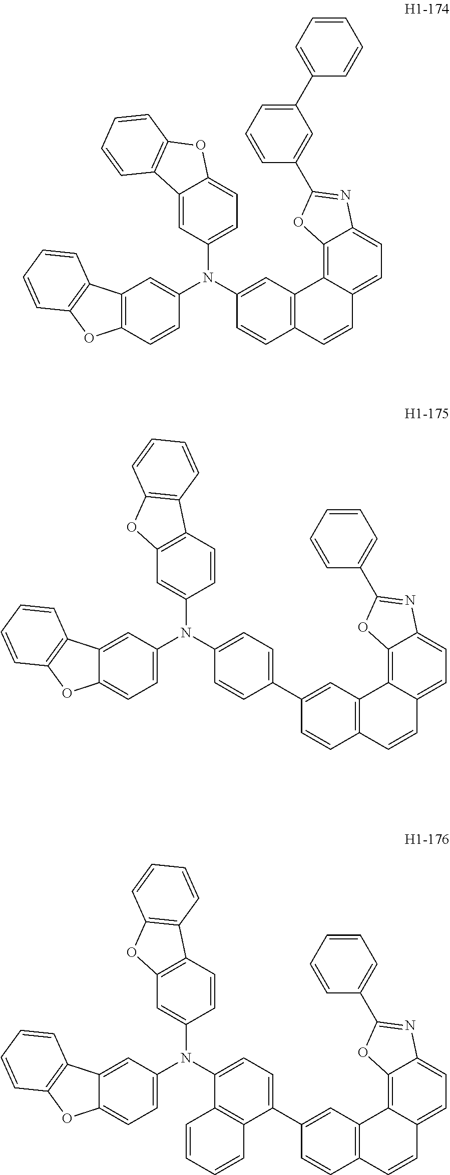 [SMILES: c1ccc(-c2cccc(-c3nc4ccc5ccc6ccc(N(c7ccc8oc9ccccc9c8c7)c7ccc8oc9ccccc9c8c7)cc6c5c4o3)c2)cc1 ; c1ccc(-c2nc3ccc4ccc5ccc(-c6ccc(N(c7ccc8c(c7)oc7ccccc78)c7ccc8oc9ccccc9c8c7)cc6)cc5c4c3o2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3o2)cc1]

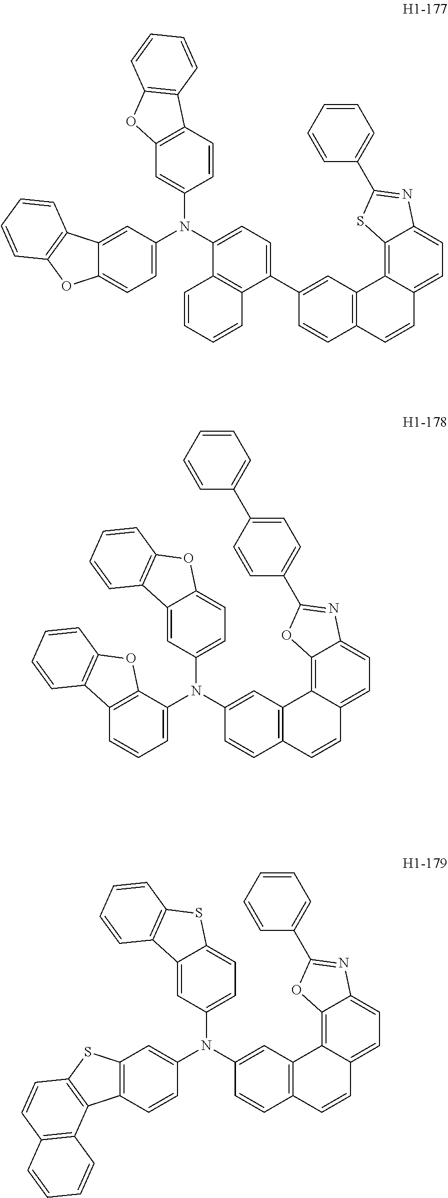 [SMILES: c1ccc(-c2nc3ccc4ccc5ccc(-c6ccc(N(c7ccc8c(c7)oc7ccccc78)c7ccc8oc9ccccc9c8c7)c7ccccc67)cc5c4c3s2)cc1 ; c1ccc(-c2ccc(-c3nc4ccc5ccc6ccc(N(c7ccc8oc9ccccc9c8c7)c7cccc8c7oc7ccccc78)cc6c5c4o3)cc2)cc1 ; c1ccc(-c2nc3ccc4ccc5ccc(N(c6ccc7c(c6)sc6ccc8ccccc8c67)c6ccc7sc8ccccc8c7c6)cc5c4c3o2)cc1]

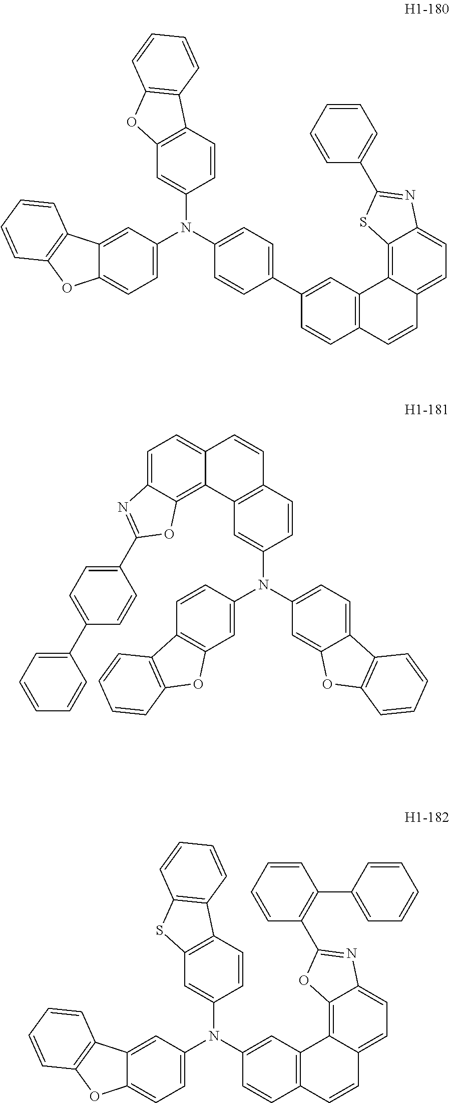

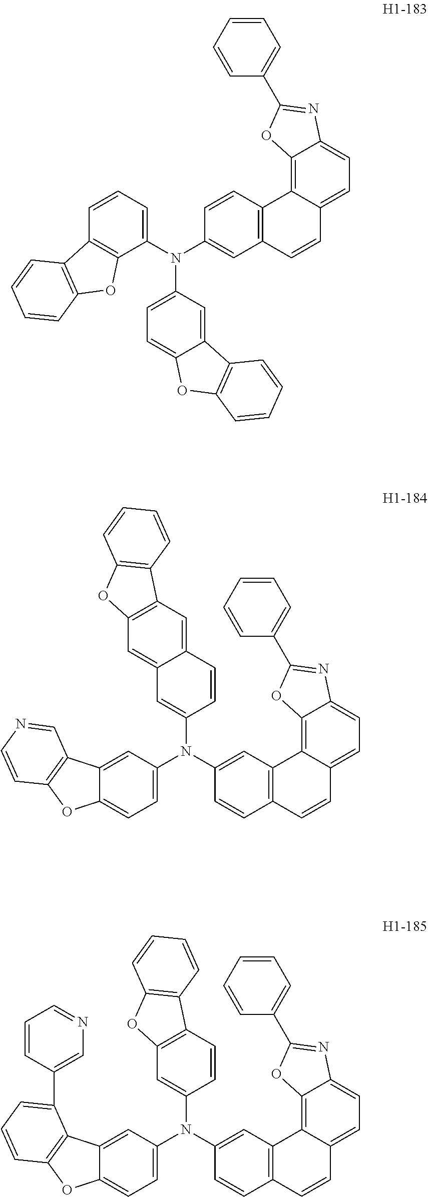 [SMILES: c1ccc(-c2nc3ccc4ccc5cc(N(c6ccc7oc8ccccc8c7c6)c6cccc7c6oc6ccccc67)ccc5c4c3o2)cc1 ; c1ccc(-c2nc3ccc4ccc5ccc(N(c6ccc7cc8c(cc7c6)oc6ccccc68)c6ccc7oc8ccncc8c7c6)cc5c4c3o2)cc1 ; c1ccc(-c2nc3ccc4ccc5ccc(N(c6ccc7c(c6)oc6ccccc67)c6ccc7oc8cccc(-c9cccnc9)c8c7c6)cc5c4c3o2)cc1]

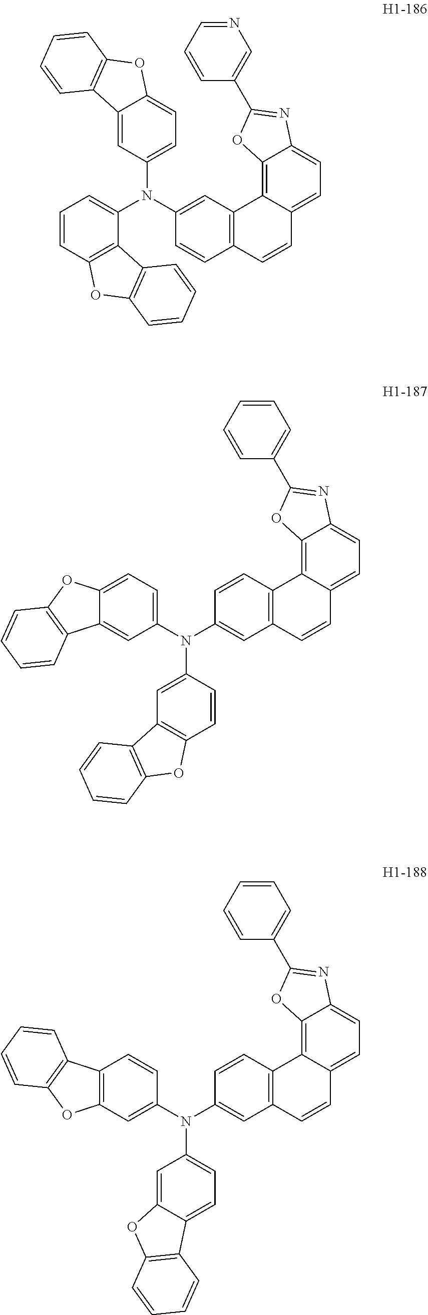 [SMILES: c1cncc(-c2nc3ccc4ccc5ccc(N(c6ccc7oc8ccccc8c7c6)c6cccc7oc8ccccc8c67)cc5c4c3o2)c1 ; c1ccc(-c2nc3ccc4ccc5cc(N(c6ccc7oc8ccccc8c7c6)c6ccc7oc8ccccc8c7c6)ccc5c4c3o2)cc1 ; c1ccc(-c2nc3ccc4ccc5cc(N(c6ccc7c(c6)oc6ccccc67)c6ccc7c(c6)oc6ccccc67)ccc5c4c3o2)cc1]

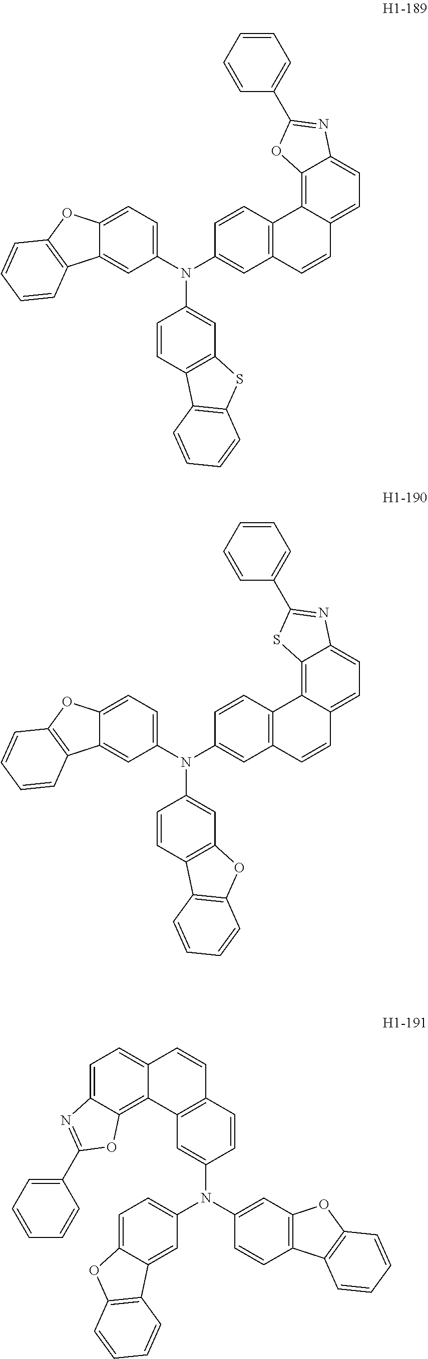 [SMILES: c1ccc(-c2nc3ccc4ccc5cc(N(c6ccc7c(c6)sc6ccccc67)c6ccc7oc8ccccc8c7c6)ccc5c4c3o2)cc1 ; c1ccc(-c2nc3ccc4ccc5cc(N(c6ccc7c(c6)oc6ccccc67)c6ccc7oc8ccccc8c7c6)ccc5c4c3s2)cc1 ; c1ccc(-c2nc3ccc4ccc5ccc(N(c6ccc7c(c6)oc6ccccc67)c6ccc7oc8ccccc8c7c6)cc5c4c3o2)cc1]

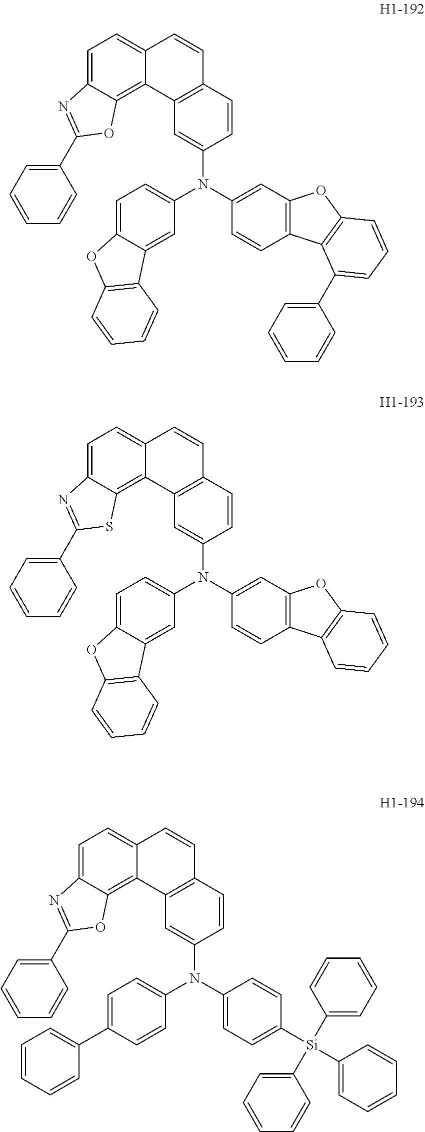 [SMILES: c1ccc(-c2nc3ccc4ccc5ccc(N(c6ccc7c(c6)oc6cccc(-c8ccccc8)c67)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3s2)cc1 ; c1ccc(-c2ccc(N(c3ccc([Si](c4ccccc4)(c4ccccc4)c4ccccc4)cc3)c3ccc4ccc5ccc6nc(-c7ccccc7)oc6c5c4c3)cc2)cc1]

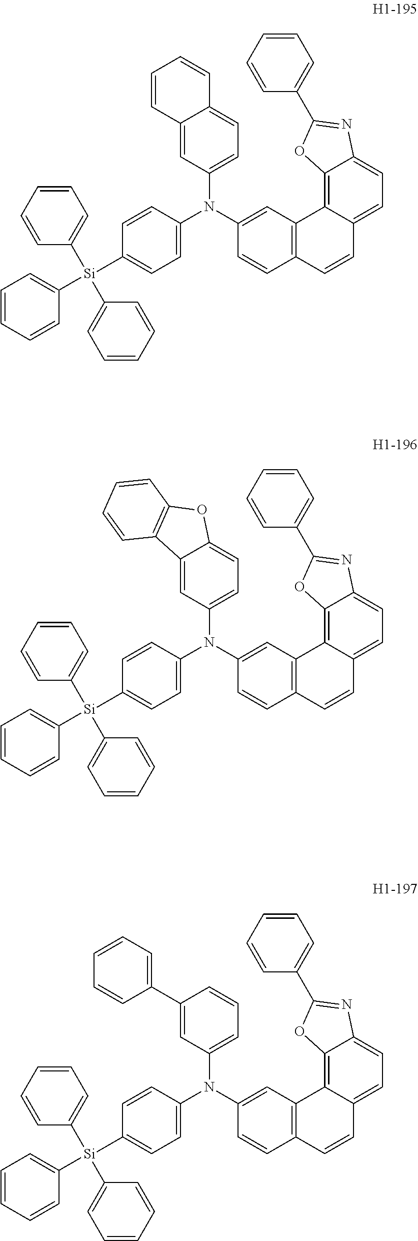 [SMILES: c1ccc(-c2nc3ccc4ccc5ccc(N(c6ccc([Si](c7ccccc7)(c7ccccc7)c7ccccc7)cc6)c6ccc7ccccc7c6)cc5c4c3o2)cc1 ; c1ccc(-c2nc3ccc4ccc5ccc(N(c6ccc([Si](c7ccccc7)(c7ccccc7)c7ccccc7)cc6)c6ccc7oc8ccccc8c7c6)cc5c4c3o2)cc1 ; c1ccc(-c2cccc(N(c3ccc([Si](c4ccccc4)(c4ccccc4)c4ccccc4)cc3)c3ccc4ccc5ccc6nc(-c7ccccc7)oc6c5c4c3)c2)cc1]

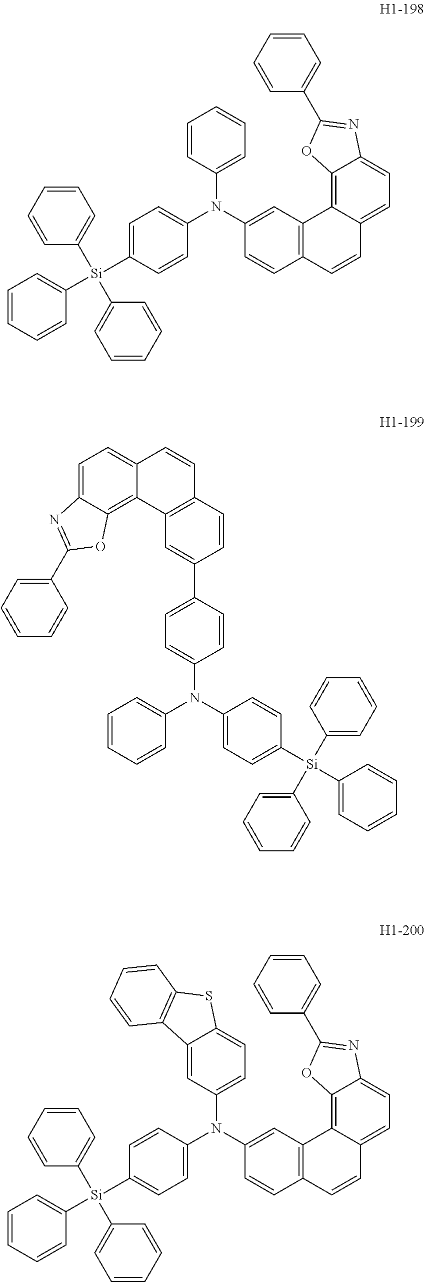 [SMILES: c1ccc(-c2nc3ccc4ccc5ccc(N(c6ccccc6)c6ccc([Si](c7ccccc7)(c7ccccc7)c7ccccc7)cc6)cc5c4c3o2)cc1 ; c1ccc(-c2nc3ccc4ccc5ccc(-c6ccc(N(c7ccccc7)c7ccc([Si](c8ccccc8)(c8ccccc8)c8ccccc8)cc7)cc6)cc5c4c3o2)cc1 ; c1ccc(-c2nc3ccc4ccc5ccc(N(c6ccc([Si](c7ccccc7)(c7ccccc7)c7ccccc7)cc6)c6ccc7sc8ccccc8c7c6)cc5c4c3o2)cc1]

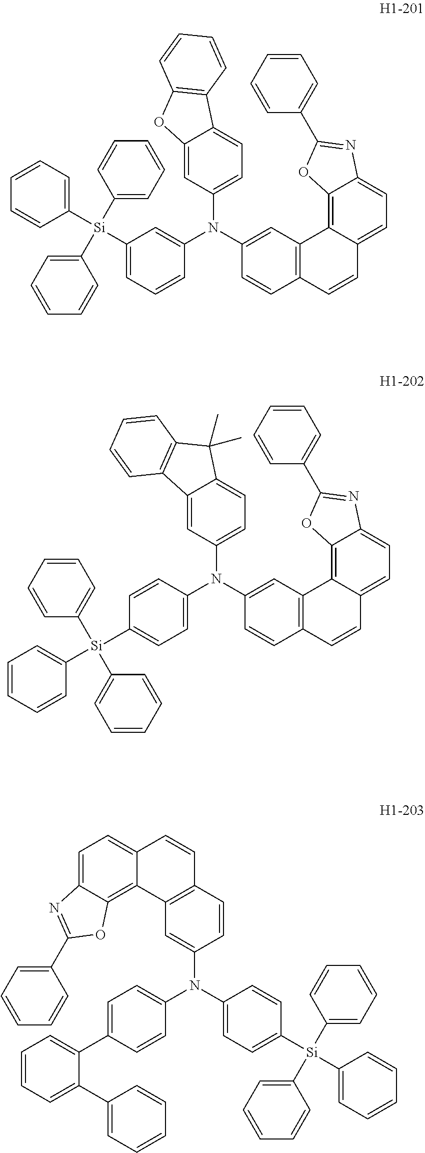 [SMILES: c1ccc(-c2nc3ccc4ccc5ccc(N(c6cccc([Si](c7ccccc7)(c7ccccc7)c7ccccc7)c6)c6ccc7c(c6)oc6ccccc67)cc5c4c3o2)cc1 ; CC1(C)c2ccccc2-c2cc(N(c3ccc([Si](c4ccccc4)(c4ccccc4)c4ccccc4)cc3)c3ccc4ccc5ccc6nc(-c7ccccc7)oc6c5c4c3)ccc21 ; c1ccc(-c2nc3ccc4ccc5ccc(N(c6ccc(-c7ccccc7-c7ccccc7)cc6)c6ccc([Si](c7ccccc7)(c7ccccc7)c7ccccc7)cc6)cc5c4c3o2)cc1]

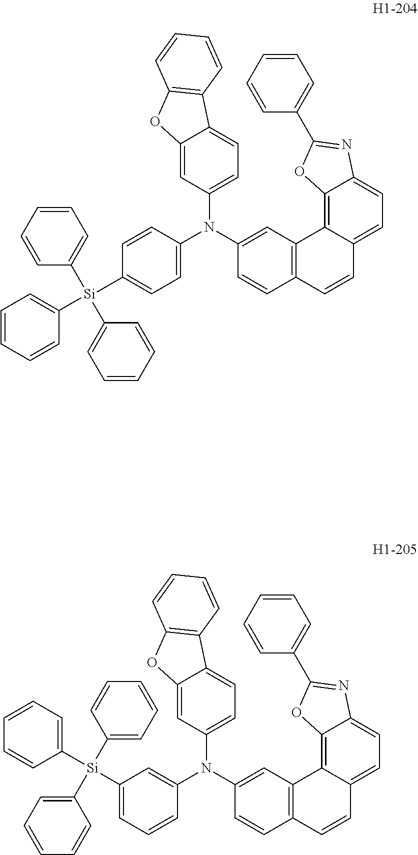 [SMILES: c1ccc(-c2nc3ccc4ccc5ccc(N(c6ccc([Si](c7ccccc7)(c7ccccc7)c7ccccc7)cc6)c6ccc7c(c6)oc6ccccc67)cc5c4c3o2)cc1 ; c1ccc(-c2nc3ccc4ccc5ccc(N(c6cccc([Si](c7ccccc7)(c7ccccc7)c7ccccc7)c6)c6ccc7c(c6)oc6ccccc67)cc5c4c3o2)cc1]

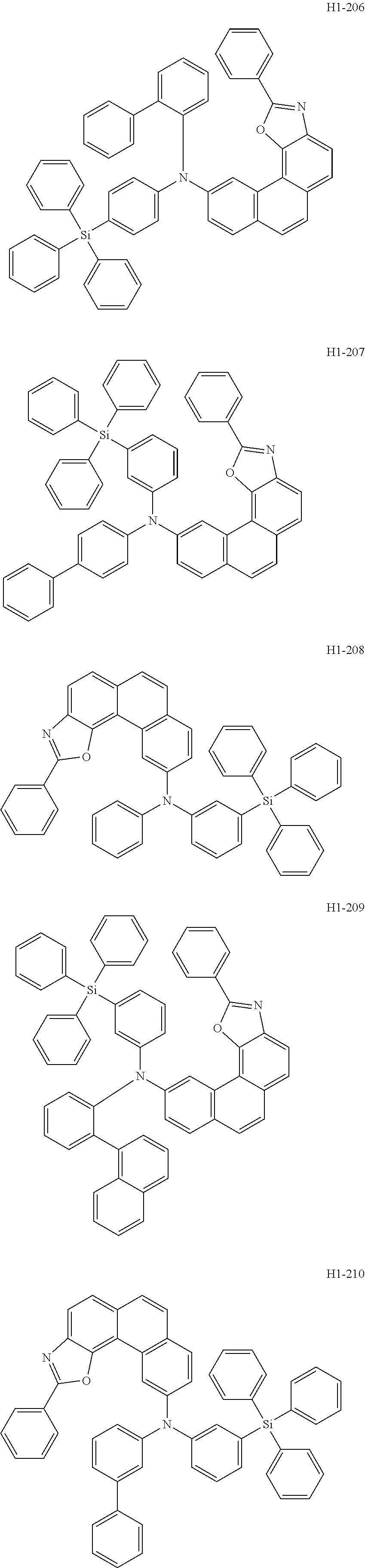

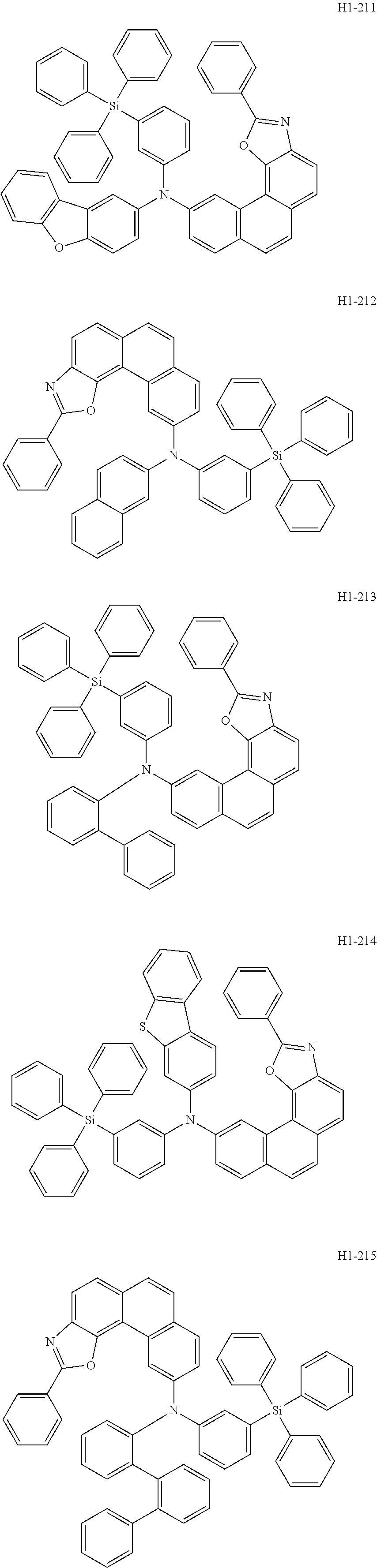

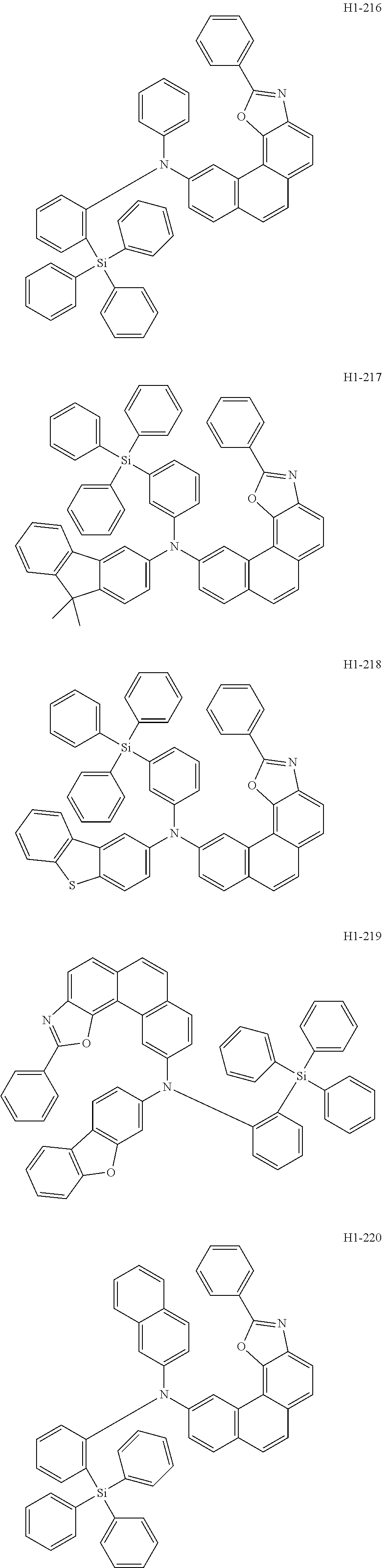

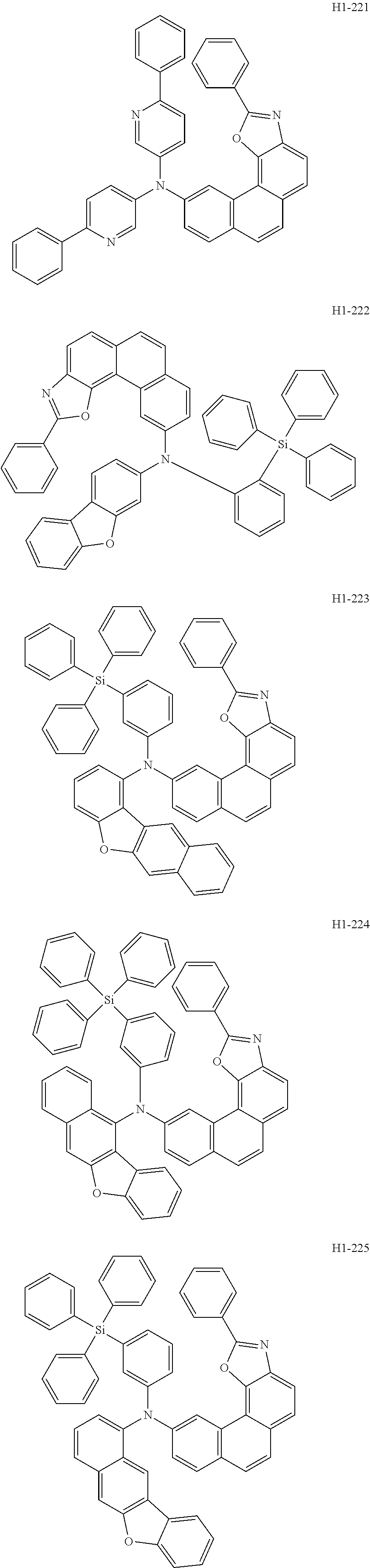

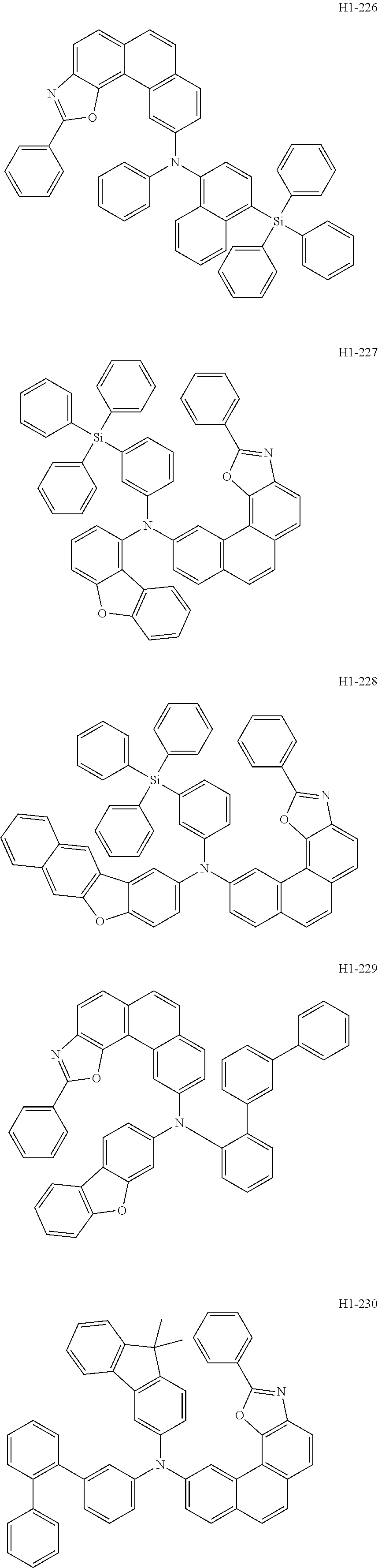

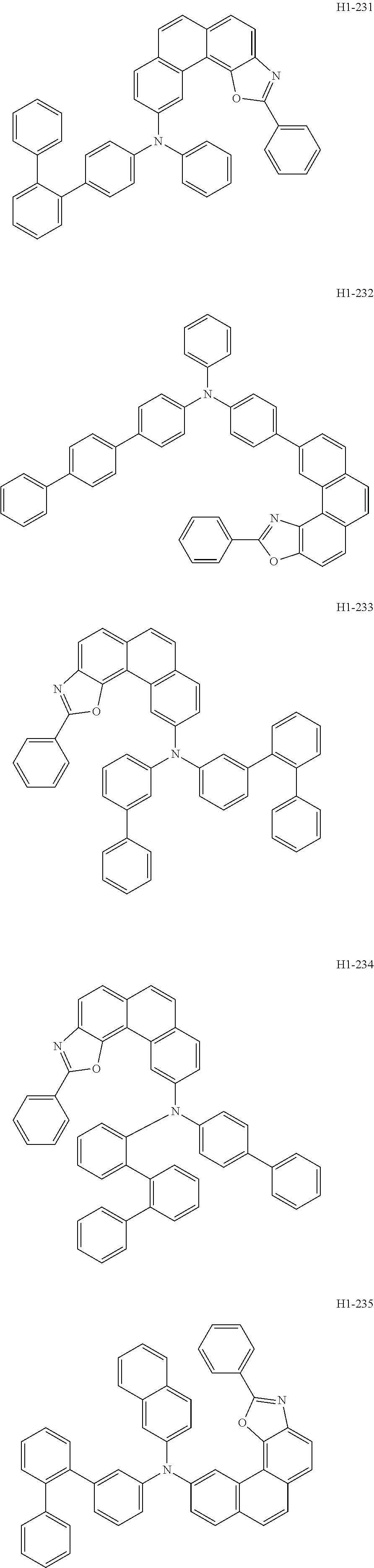 [SMILES: c1ccc(-c2nc3ccc4ccc5ccc(N(c6ccccc6)c6ccc(-c7ccccc7-c7ccccc7)cc6)cc5c4c3o2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2cccc(N(c3cccc(-c4ccccc4-c4ccccc4)c3)c3ccc4ccc5ccc6nc(-c7ccccc7)oc6c5c4c3)c2)cc1 ; c1ccc(-c2ccc(N(c3ccc4ccc5ccc6nc(-c7ccccc7)oc6c5c4c3)c3ccccc3-c3ccccc3-c3ccccc3)cc2)cc1 ; c1ccc(-c2nc3ccc4ccc5ccc(N(c6cccc(-c7ccccc7-c7ccccc7)c6)c6ccc7ccccc7c6)cc5c4c3o2)cc1]

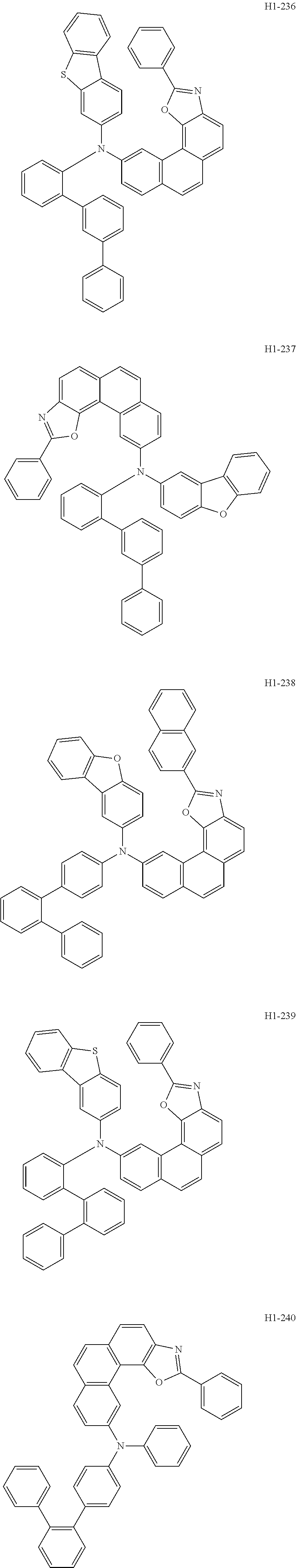 [SMILES: c1ccc(-c2cccc(-c3ccccc3N(c3ccc4c(c3)sc3ccccc34)c3ccc4ccc5ccc6nc(-c7ccccc7)oc6c5c4c3)c2)cc1 ; c1ccc(-c2cccc(-c3ccccc3N(c3ccc4oc5ccccc5c4c3)c3ccc4ccc5ccc6nc(-c7ccccc7)oc6c5c4c3)c2)cc1 ; c1ccc(-c2ccccc2-c2ccc(N(c3ccc4oc5ccccc5c4c3)c3ccc4ccc5ccc6nc(-c7ccc8ccccc8c7)oc6c5c4c3)cc2)cc1 ; c1ccc(-c2nc3ccc4ccc5ccc(N(c6ccc7sc8ccccc8c7c6)c6ccccc6-c6ccccc6-c6ccccc6)cc5c4c3o2)cc1 ; c1ccc(-c2nc3ccc4ccc5ccc(N(c6ccccc6)c6ccc(-c7ccccc7-c7ccccc7)cc6)cc5c4c3o2)cc1]

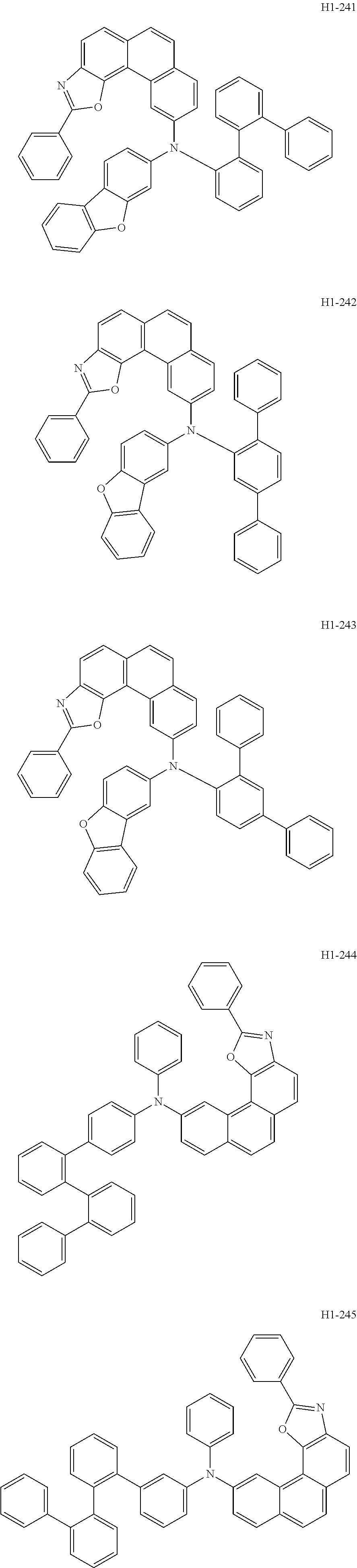 [SMILES: c1ccc(-c2nc3ccc4ccc5ccc(N(c6ccc7c(c6)oc6ccccc67)c6ccccc6-c6ccccc6-c6ccccc6)cc5c4c3o2)cc1 ; c1ccc(-c2ccc(-c3ccccc3)c(N(c3ccc4oc5ccccc5c4c3)c3ccc4ccc5ccc6nc(-c7ccccc7)oc6c5c4c3)c2)cc1 ; c1ccc(-c2ccc(N(c3ccc4oc5ccccc5c4c3)c3ccc4ccc5ccc6nc(-c7ccccc7)oc6c5c4c3)c(-c3ccccc3)c2)cc1 ; c1ccc(-c2nc3ccc4ccc5ccc(N(c6ccccc6)c6ccc(-c7ccccc7-c7ccccc7-c7ccccc7)cc6)cc5c4c3o2)cc1 ; c1ccc(-c2nc3ccc4ccc5ccc(N(c6ccccc6)c6cccc(-c7ccccc7-c7ccccc7-c7ccccc7)c6)cc5c4c3o2)cc1]

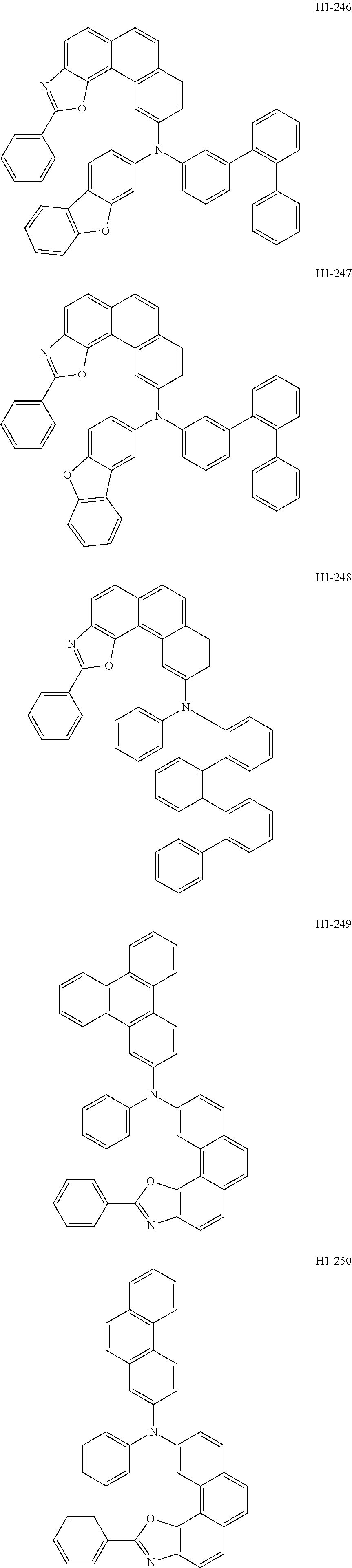 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1 ; c1ccc(-c2nc3ccc4ccc5ccc(N(c6cccc(-c7ccccc7-c7ccccc7)c6)c6ccc7oc8ccccc8c7c6)cc5c4c3o2)cc1 ; c1ccc(-c2nc3ccc4ccc5ccc(N(c6ccccc6)c6ccccc6-c6ccccc6-c6ccccc6-c6ccccc6)cc5c4c3o2)cc1 ; c1ccc(-c2nc3ccc4ccc5ccc(N(c6ccccc6)c6ccc7c8ccccc8c8ccccc8c7c6)cc5c4c3o2)cc1 ; c1ccc(-c2nc3ccc4ccc5ccc(N(c6ccccc6)c6ccc7c(ccc8ccccc87)c6)cc5c4c3o2)cc1]

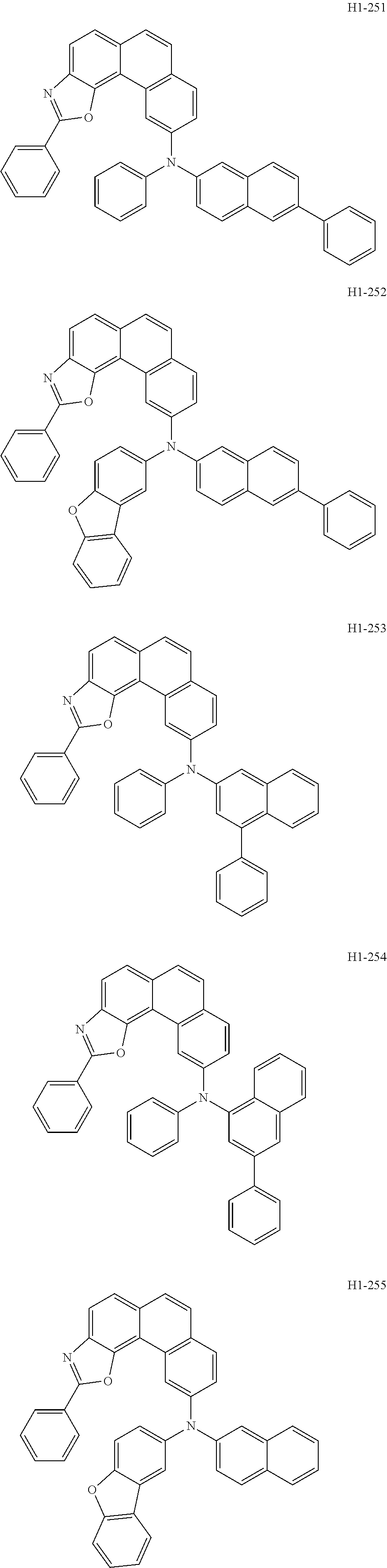 [SMILES: c1ccc(-c2ccc3cc(N(c4ccccc4)c4ccc5ccc6ccc7nc(-c8ccccc8)oc7c6c5c4)ccc3c2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1 ; c1ccc(-c2nc3ccc4ccc5ccc(N(c6ccccc6)c6cc(-c7ccccc7)c7ccccc7c6)cc5c4c3o2)cc1 ; c1ccc(-c2cc(N(c3ccccc3)c3ccc4ccc5ccc6nc(-c7ccccc7)oc6c5c4c3)c3ccccc3c2)cc1 ; c1ccc(-c2nc3ccc4ccc5ccc(N(c6ccc7ccccc7c6)c6ccc7oc8ccccc8c7c6)cc5c4c3o2)cc1]

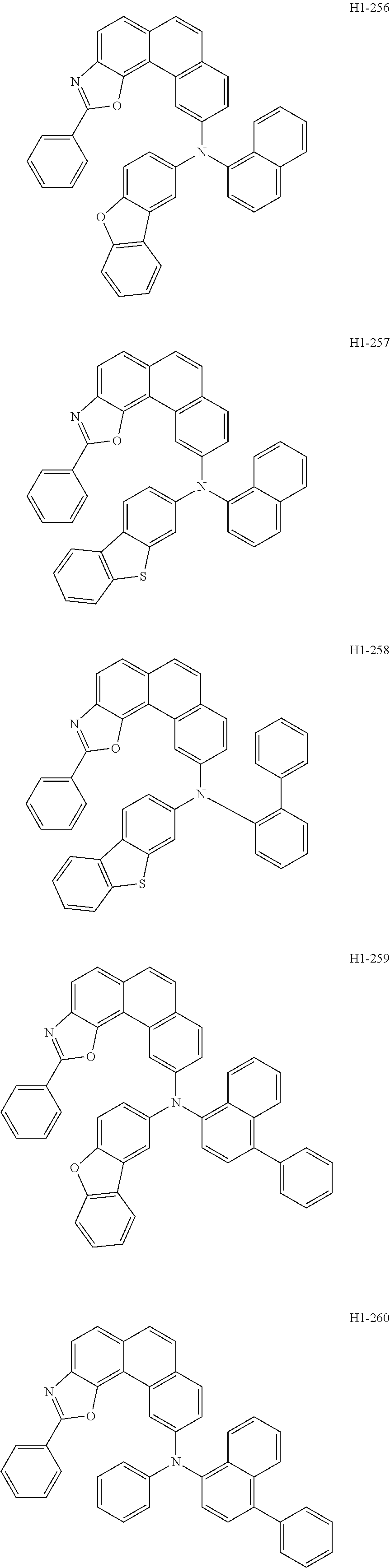 [SMILES: c1ccc(-c2nc3ccc4ccc5ccc(N(c6ccc7oc8ccccc8c7c6)c6cccc7ccccc67)cc5c4c3o2)cc1 ; c1ccc(-c2nc3ccc4ccc5ccc(N(c6ccc7c(c6)sc6ccccc67)c6cccc7ccccc67)cc5c4c3o2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(N(c6ccc7oc8ccccc8c7c6)c6ccc(-c7ccccc7)c7ccccc67)cc5c4c3o2)cc1 ; c1ccc(-c2nc3ccc4ccc5ccc(N(c6ccccc6)c6ccc(-c7ccccc7)c7ccccc67)cc5c4c3o2)cc1]

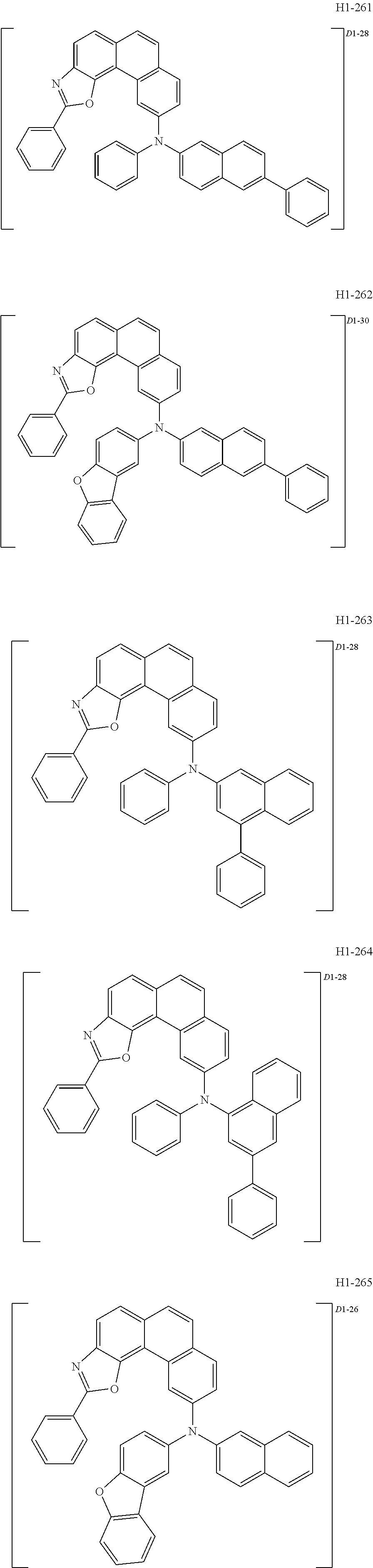

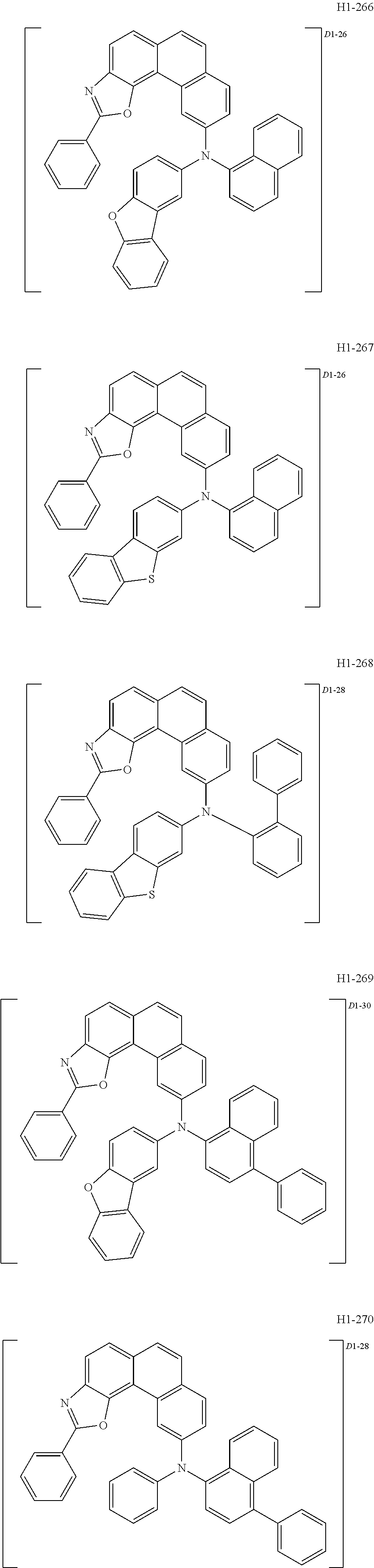

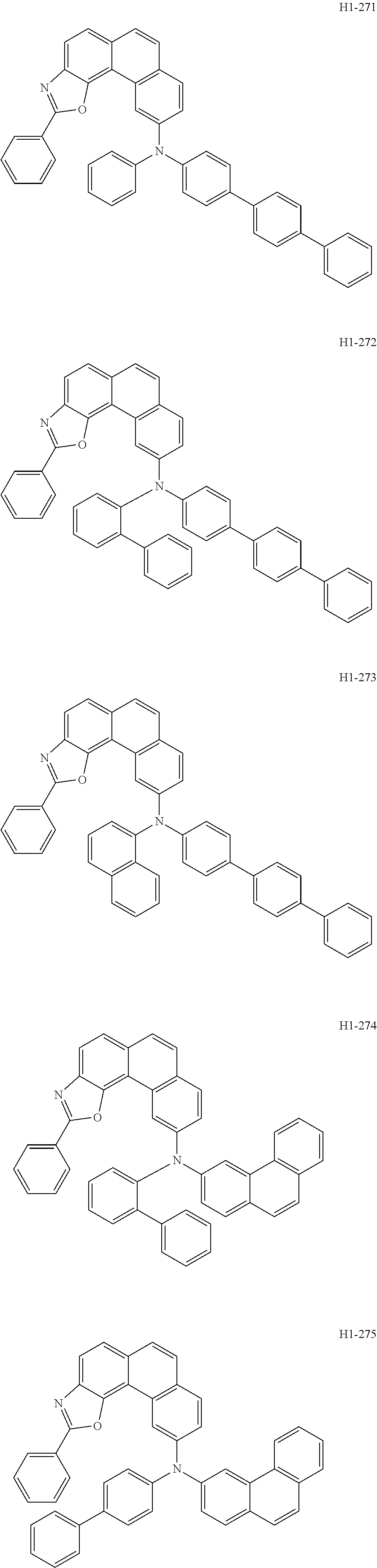 [SMILES: c1ccc(-c2ccc(-c3ccc(N(c4ccccc4)c4ccc5ccc6ccc7nc(-c8ccccc8)oc7c6c5c4)cc3)cc2)cc1 ; c1ccc(-c2ccc(-c3ccc(N(c4ccc5ccc6ccc7nc(-c8ccccc8)oc7c6c5c4)c4ccccc4-c4ccccc4)cc3)cc2)cc1 ; c1ccc(-c2ccc(-c3ccc(N(c4ccc5ccc6ccc7nc(-c8ccccc8)oc7c6c5c4)c4cccc5ccccc45)cc3)cc2)cc1 ; c1ccc(-c2nc3ccc4ccc5ccc(N(c6ccc7ccc8ccccc8c7c6)c6ccccc6-c6ccccc6)cc5c4c3o2)cc1 ; c1ccc(-c2ccc(N(c3ccc4ccc5ccccc5c4c3)c3ccc4ccc5ccc6nc(-c7ccccc7)oc6c5c4c3)cc2)cc1]

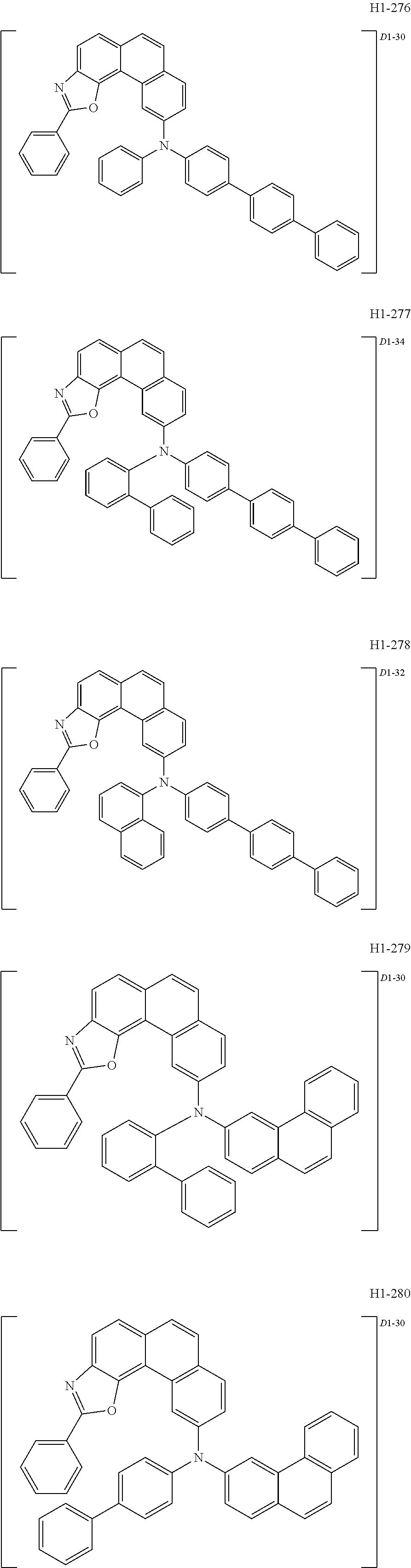

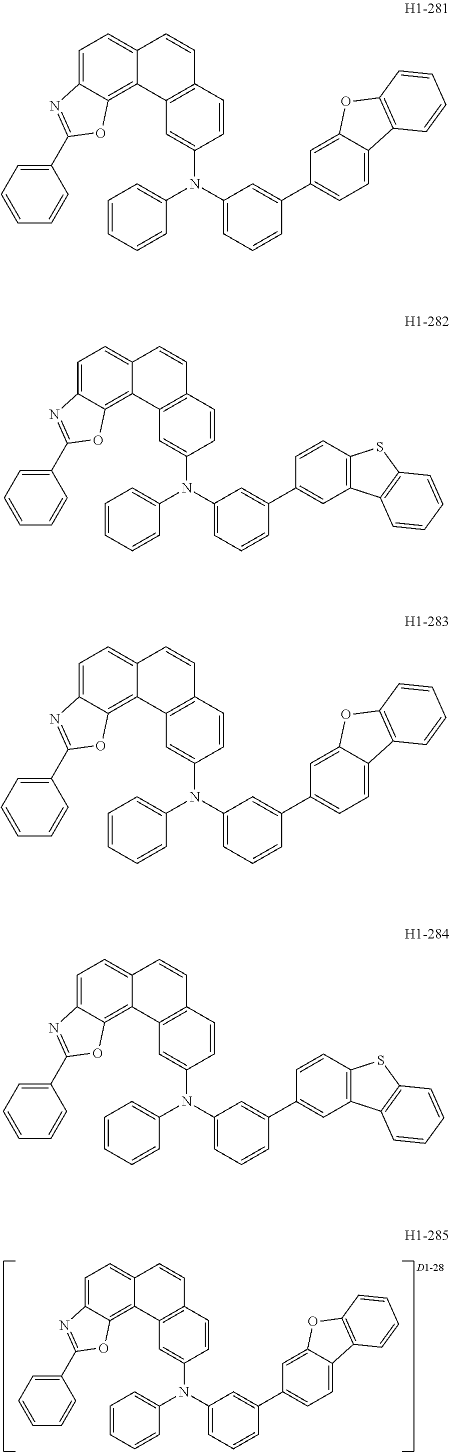

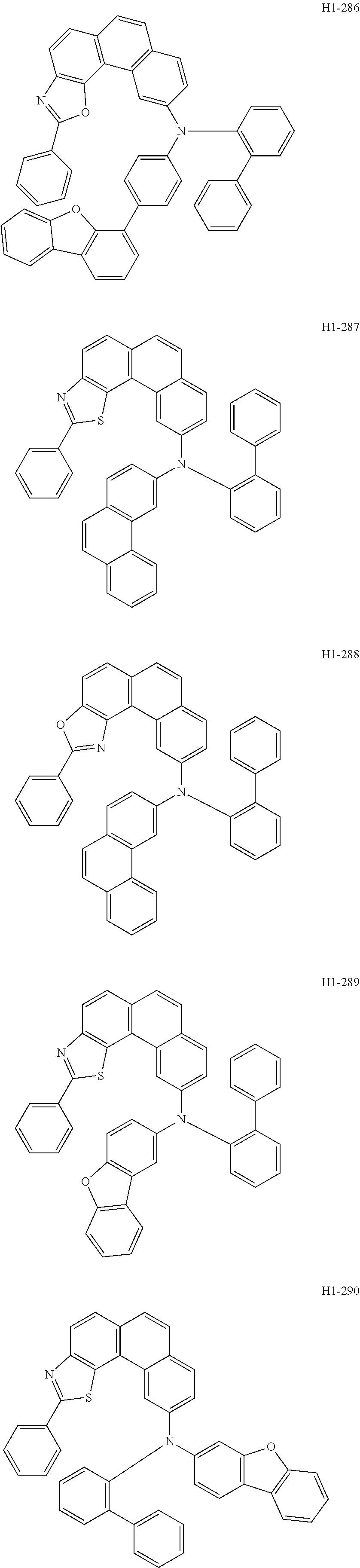 [SMILES: c1ccc(-c2nc3ccc4ccc5ccc(N(c6ccc(-c7cccc8c7oc7ccccc78)cc6)c6ccccc6-c6ccccc6)cc5c4c3o2)cc1 ; c1ccc(-c2nc3ccc4ccc5ccc(N(c6ccc7ccc8ccccc8c7c6)c6ccccc6-c6ccccc6)cc5c4c3s2)cc1 ; c1ccc(-c2nc3c(ccc4ccc5ccc(N(c6ccc7ccc8ccccc8c7c6)c6ccccc6-c6ccccc6)cc5c43)o2)cc1 ; c1ccc(-c2nc3ccc4ccc5ccc(N(c6ccc7oc8ccccc8c7c6)c6ccccc6-c6ccccc6)cc5c4c3s2)cc1 ; c1ccc(-c2nc3ccc4ccc5ccc(N(c6ccc7c(c6)oc6ccccc67)c6ccccc6-c6ccccc6)cc5c4c3s2)cc1]

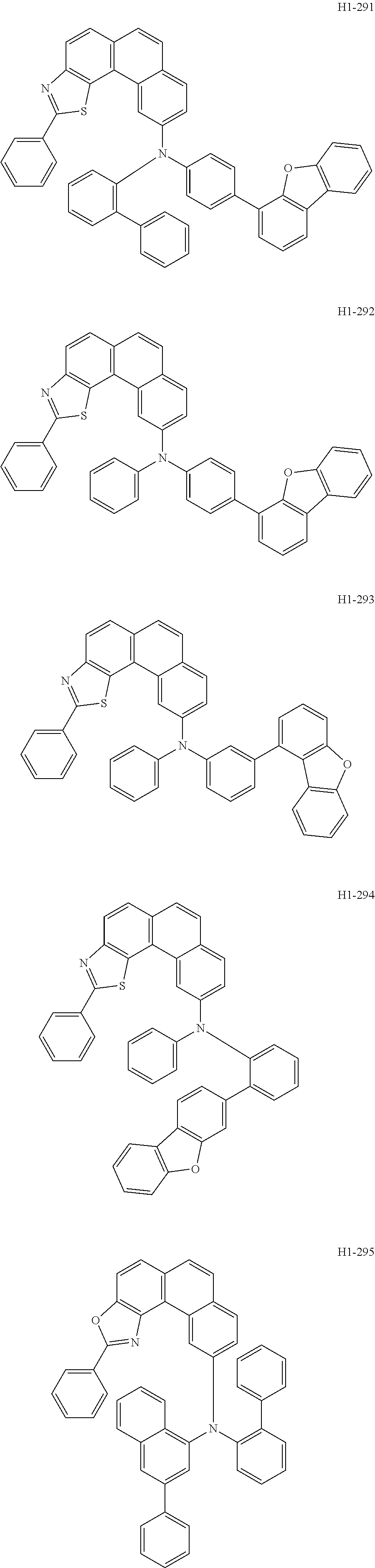 [SMILES: c1ccc(-c2nc3ccc4ccc5ccc(N(c6ccc(-c7cccc8c7oc7ccccc78)cc6)c6ccccc6-c6ccccc6)cc5c4c3s2)cc1 ; c1ccc(-c2nc3ccc4ccc5ccc(N(c6ccccc6)c6ccc(-c7cccc8c7oc7ccccc78)cc6)cc5c4c3s2)cc1 ; c1ccc(-c2nc3ccc4ccc5ccc(N(c6ccccc6)c6cccc(-c7cccc8oc9ccccc9c78)c6)cc5c4c3s2)cc1 ; c1ccc(-c2nc3ccc4ccc5ccc(N(c6ccccc6)c6ccccc6-c6ccc7c(c6)oc6ccccc67)cc5c4c3s2)cc1 ; c1ccc(-c2cc(N(c3ccc4ccc5ccc6oc(-c7ccccc7)nc6c5c4c3)c3ccccc3-c3ccccc3)c3ccccc3c2)cc1]

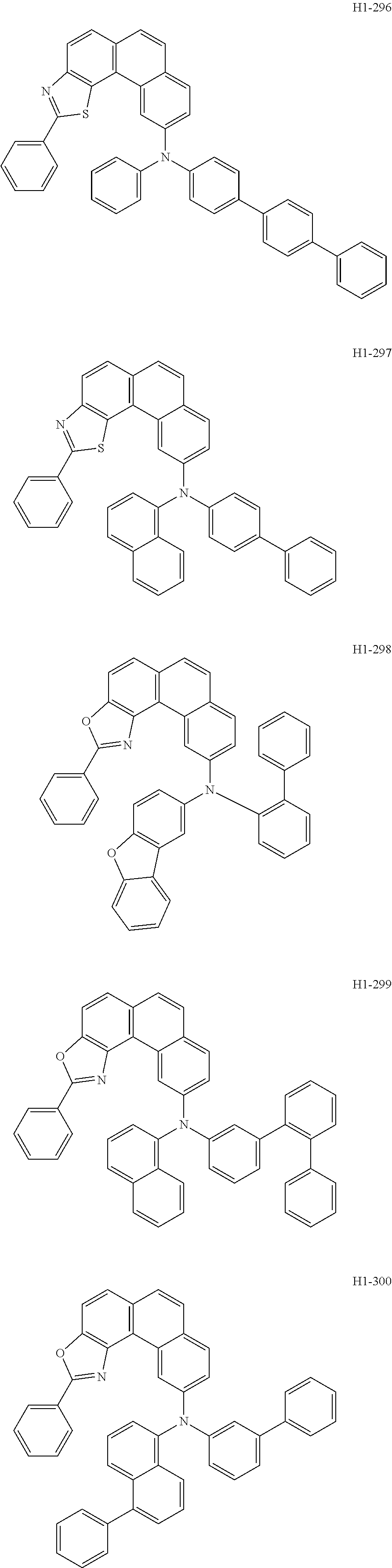 [SMILES: c1ccc(-c2ccc(-c3ccc(N(c4ccccc4)c4ccc5ccc6ccc7nc(-c8ccccc8)sc7c6c5c4)cc3)cc2)cc1 ; c1ccc(-c2ccc(N(c3ccc4ccc5ccc6nc(-c7ccccc7)sc6c5c4c3)c3cccc4ccccc34)cc2)cc1 ; c1ccc(-c2nc3c(ccc4ccc5ccc(N(c6ccc7oc8ccccc8c7c6)c6ccccc6-c6ccccc6)cc5c43)o2)cc1 ; c1ccc(-c2nc3c(ccc4ccc5ccc(N(c6cccc(-c7ccccc7-c7ccccc7)c6)c6cccc7ccccc67)cc5c43)o2)cc1 ; c1ccc(-c2cccc(N(c3ccc4ccc5ccc6oc(-c7ccccc7)nc6c5c4c3)c3cccc4c(-c5ccccc5)cccc34)c2)cc1]

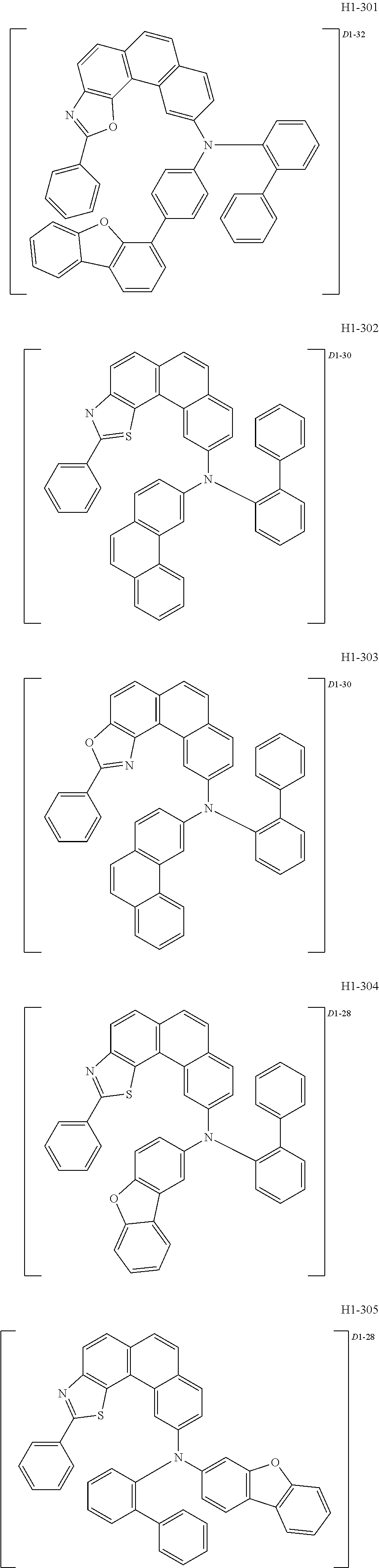

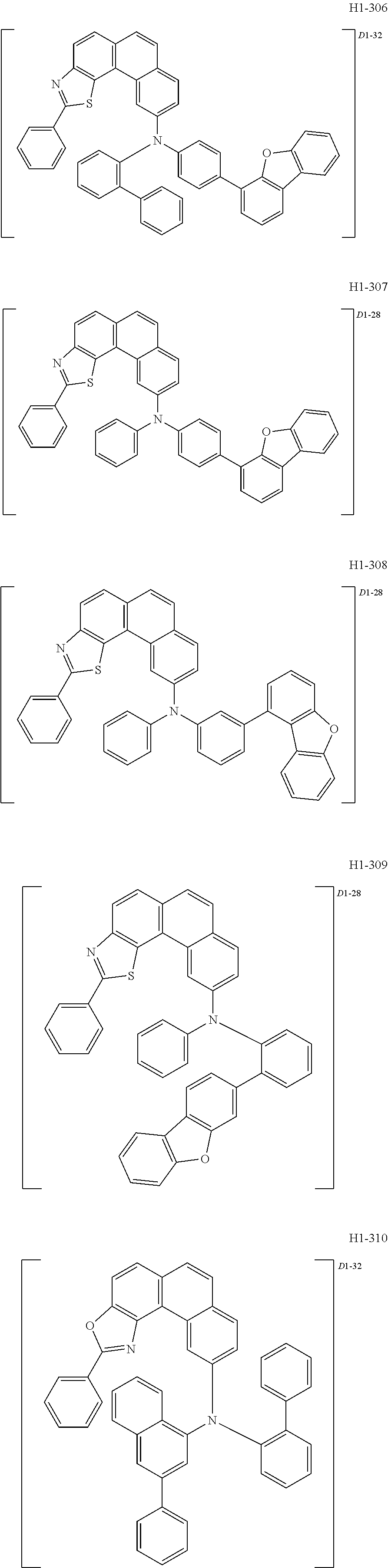

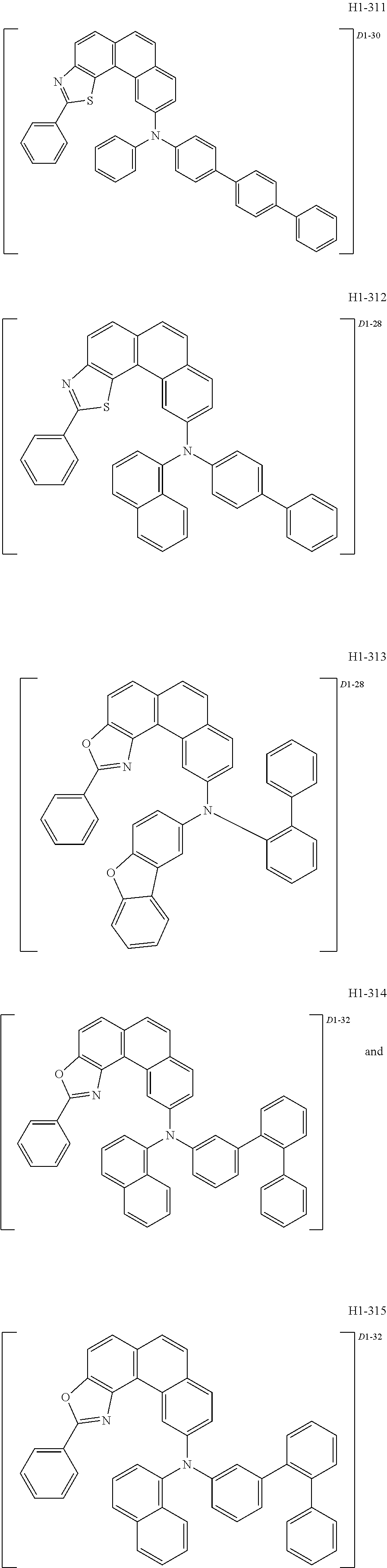

8. The plurality of host materials according to claim 1, wherein the compound represented by formula 2 is selected from the following compounds:

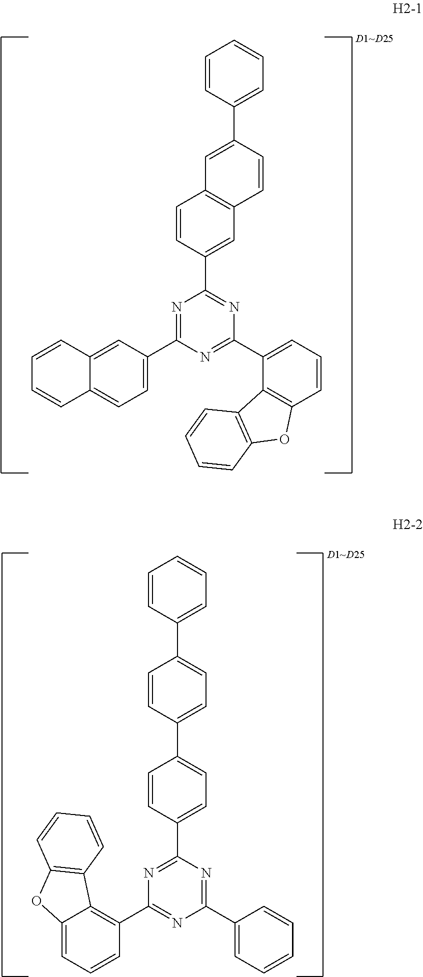

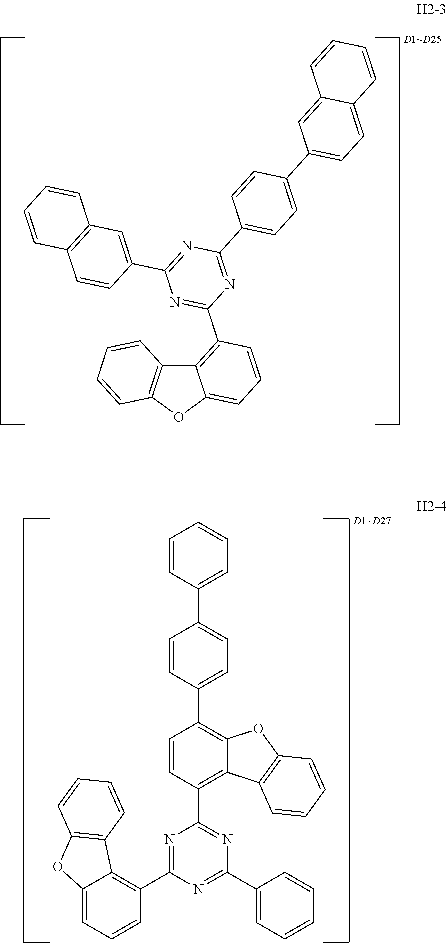

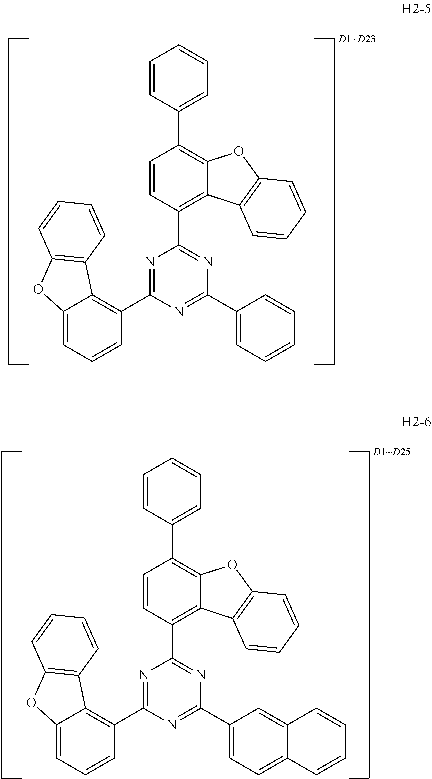

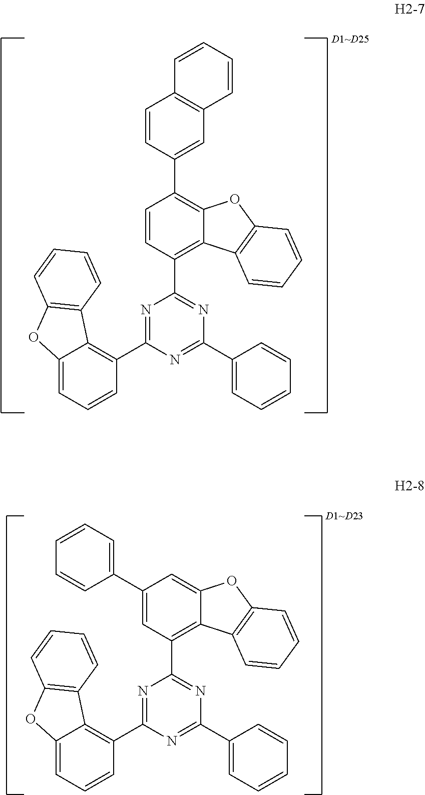

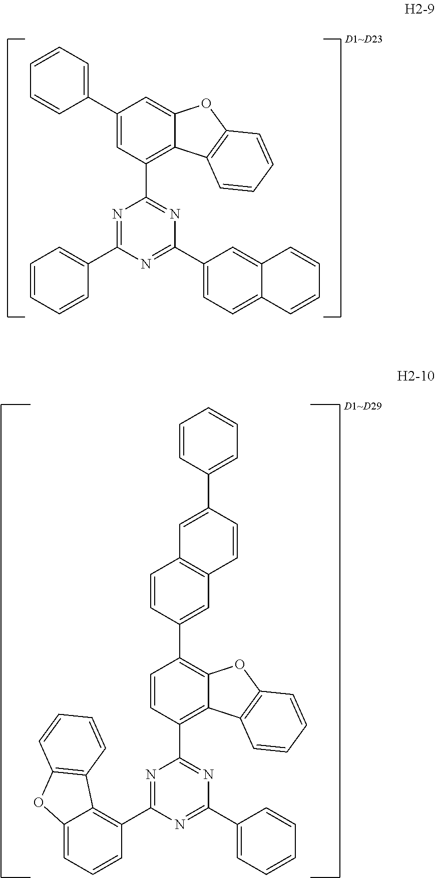

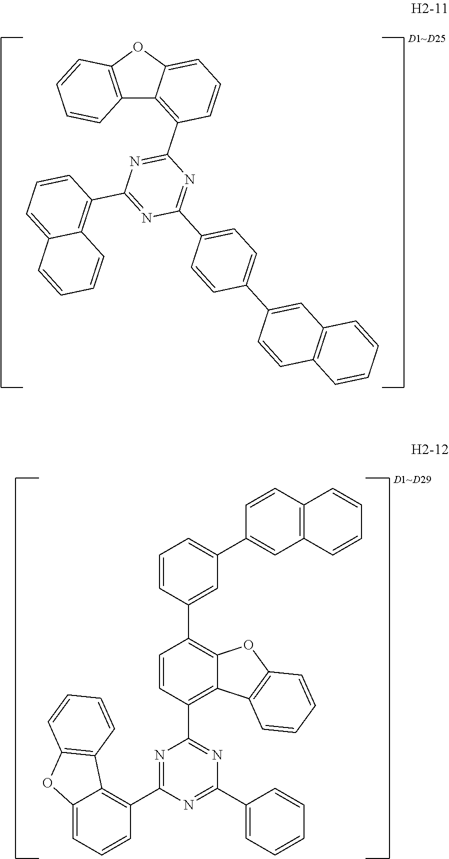

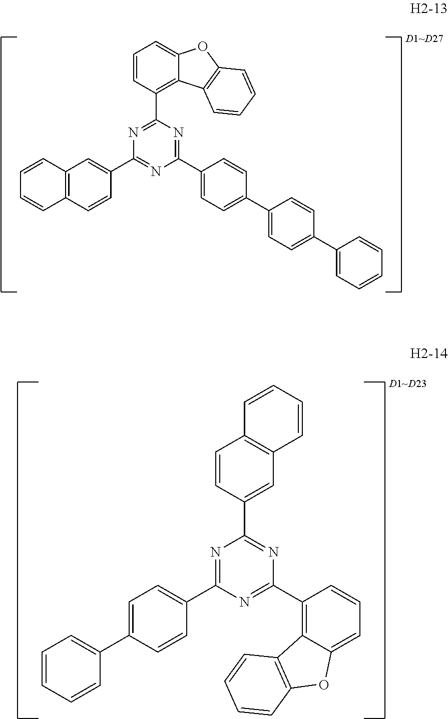

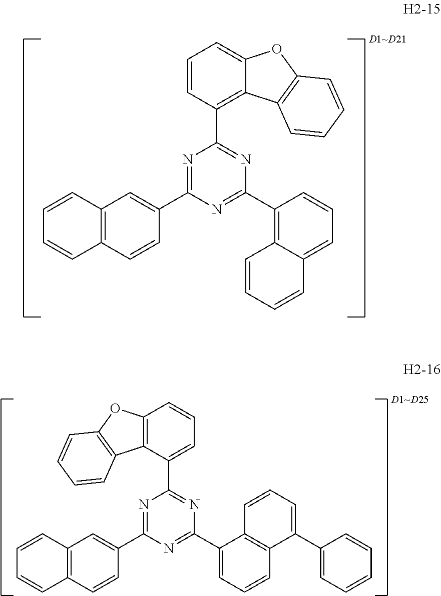

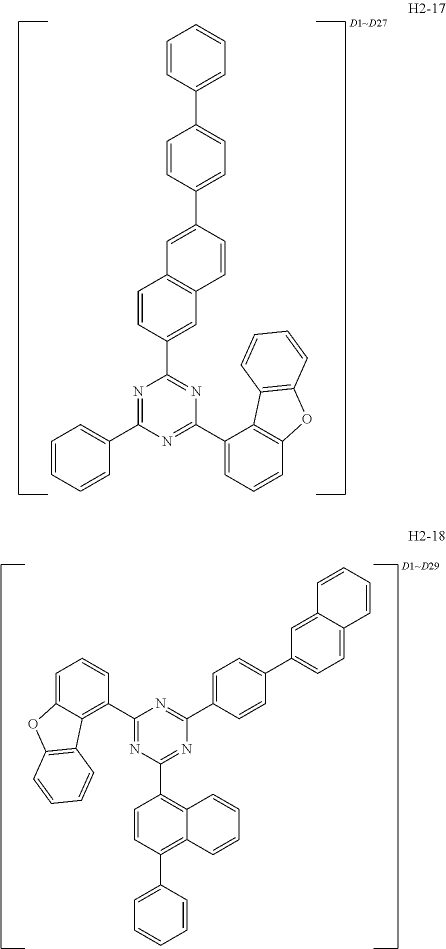

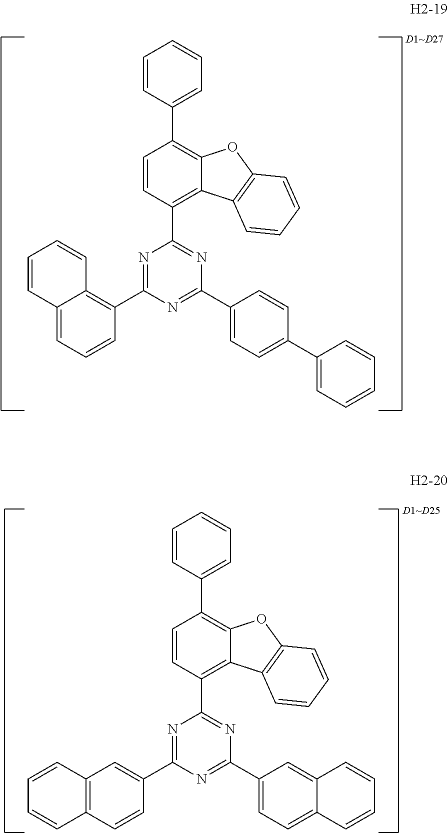

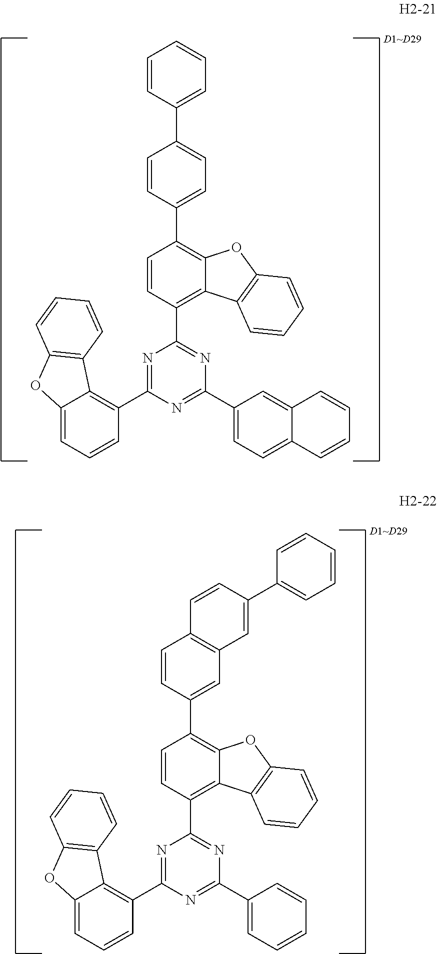

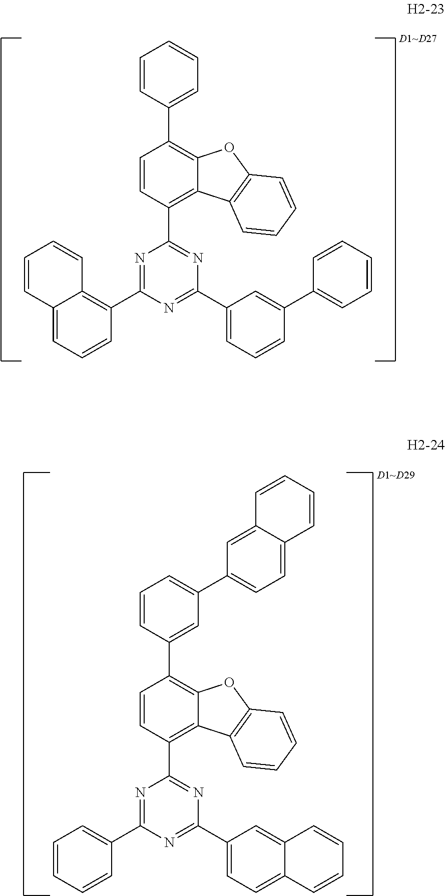

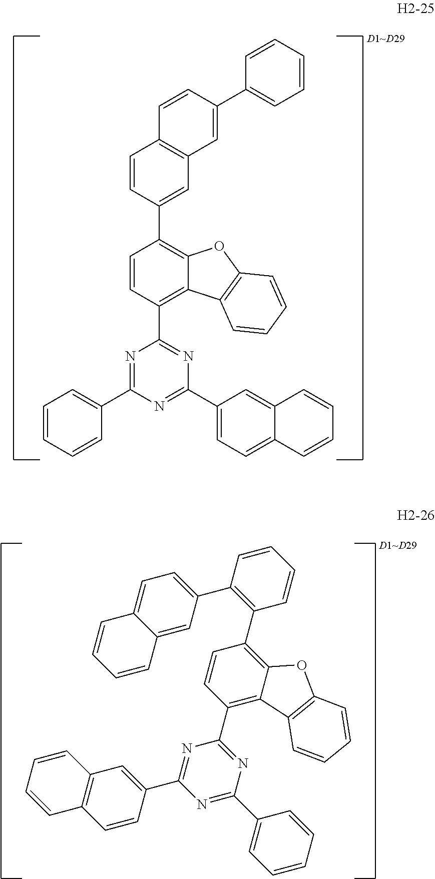

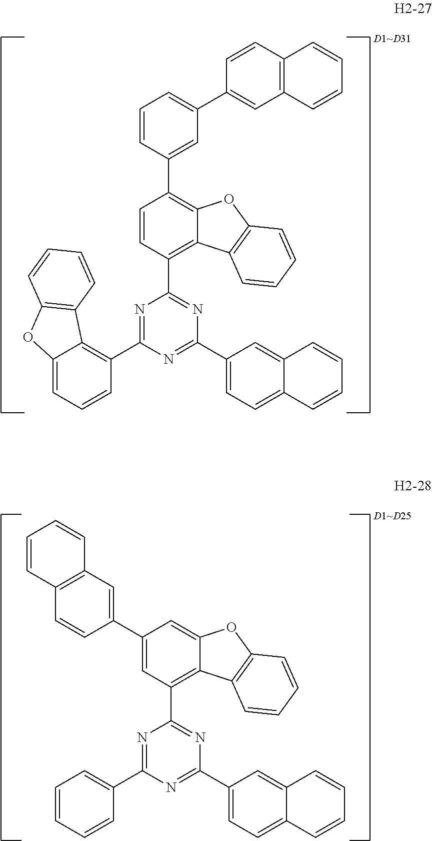

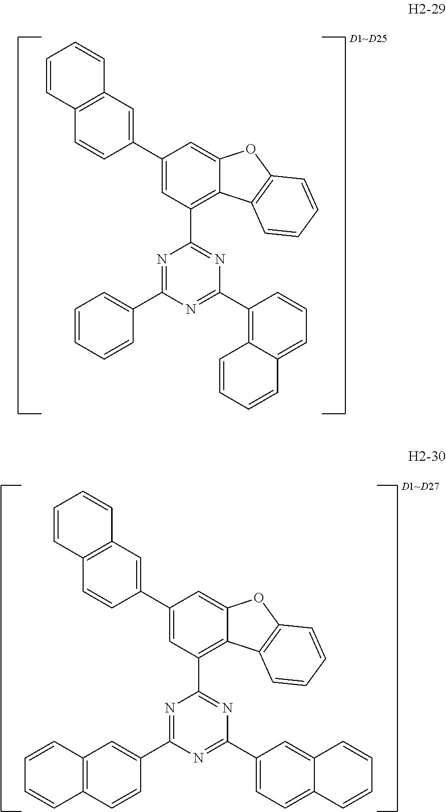

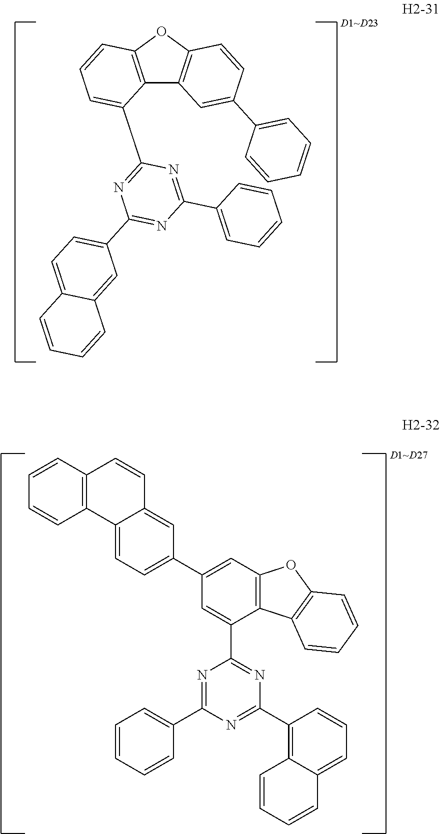

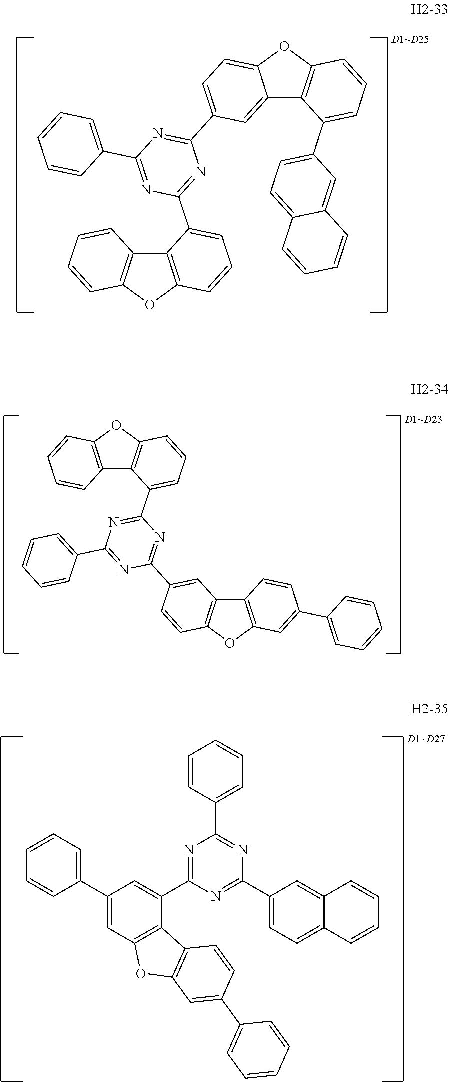

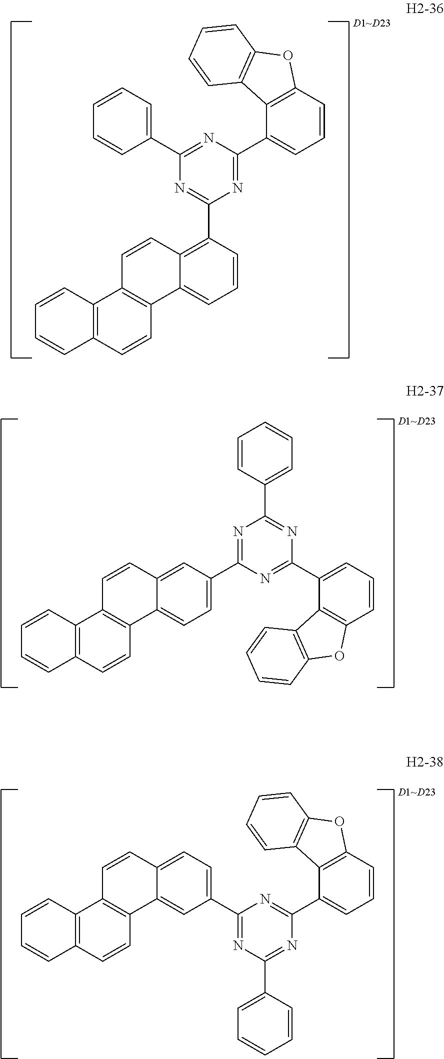

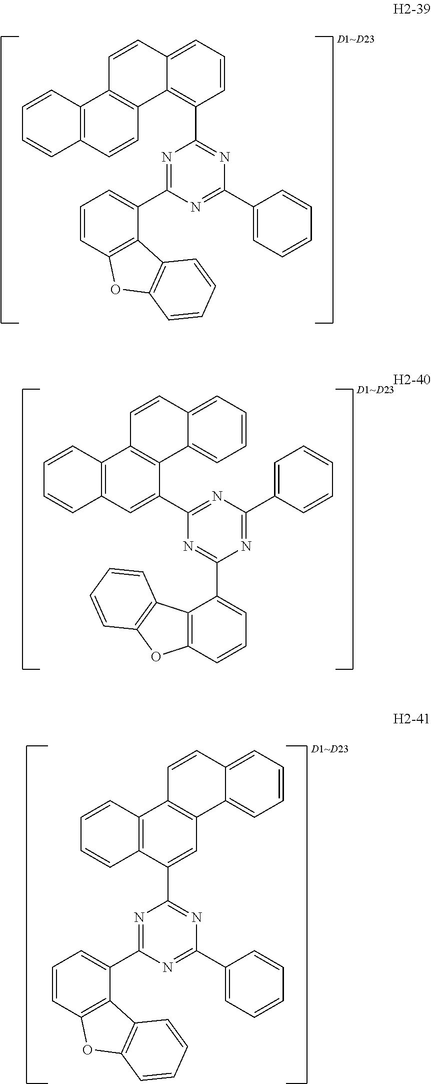

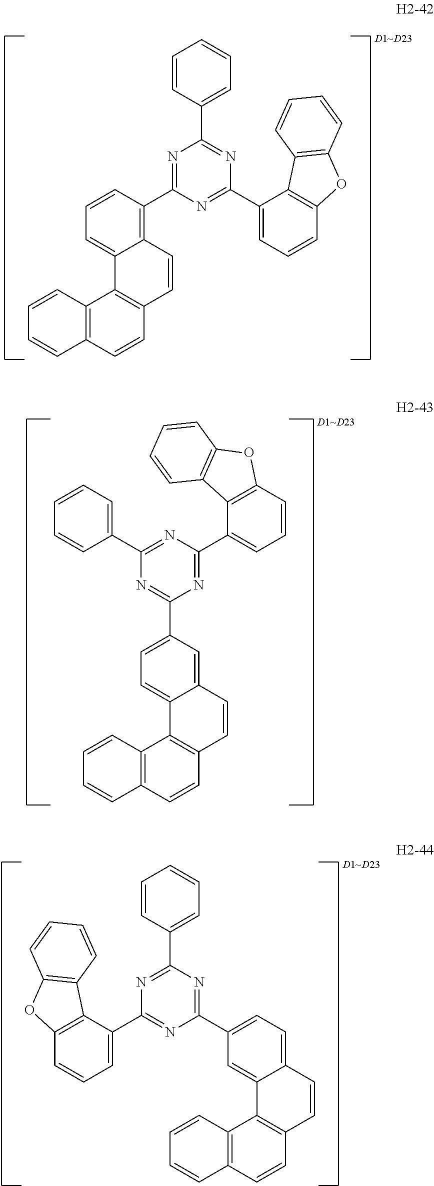

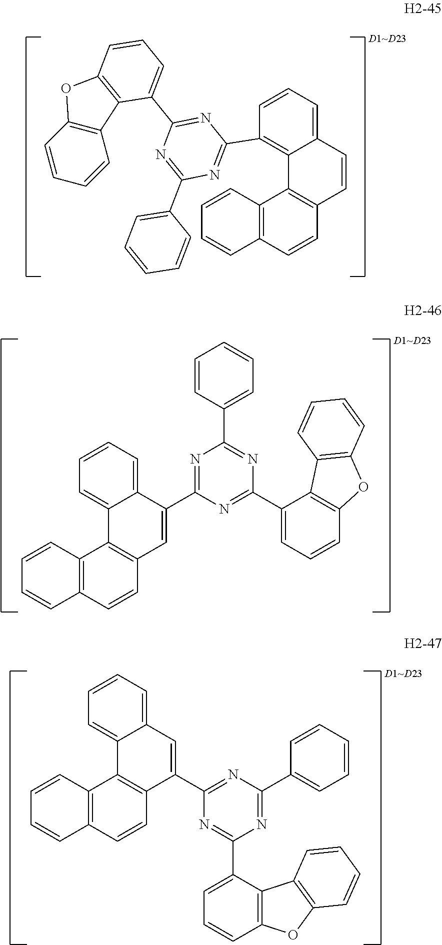

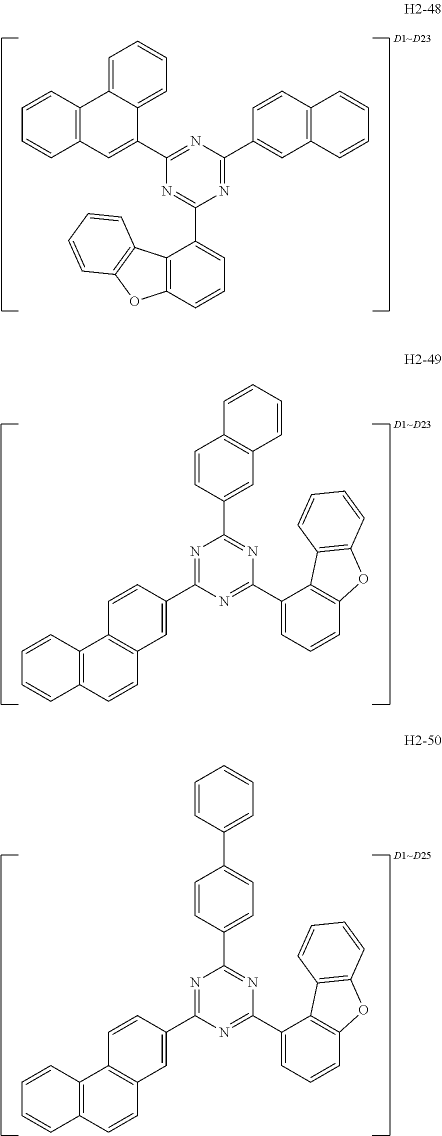

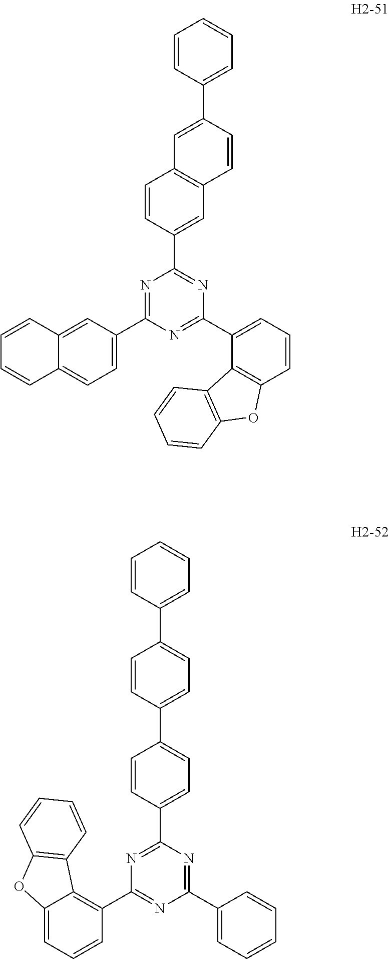 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccccc5)nc(-c5cccc6oc7ccccc7c56)n4)cc3)cc2)cc1]

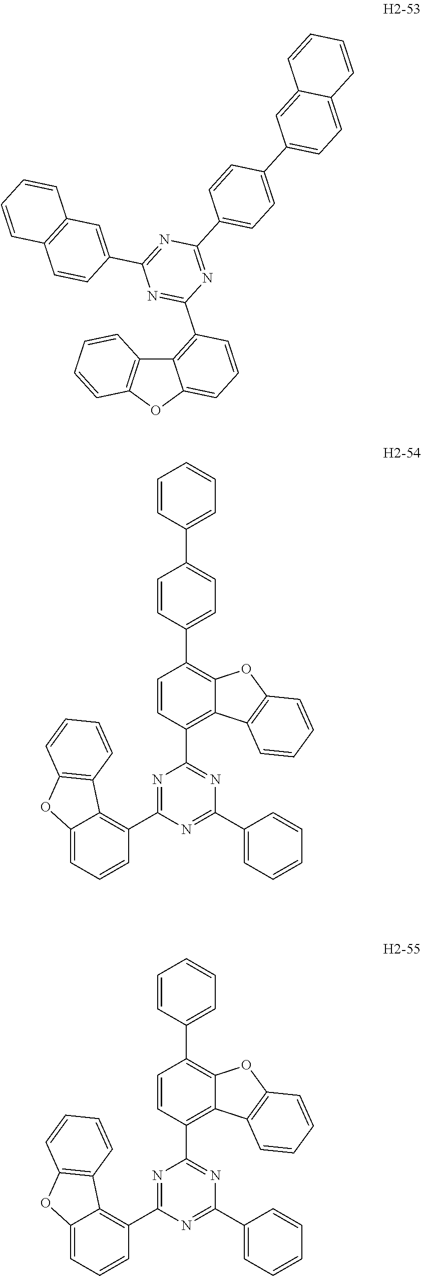 [SMILES: c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2nc(-c3cccc4oc5ccccc5c34)nc(-c3ccc(-c4ccccc4)c4oc5ccccc5c34)n2)cc1]

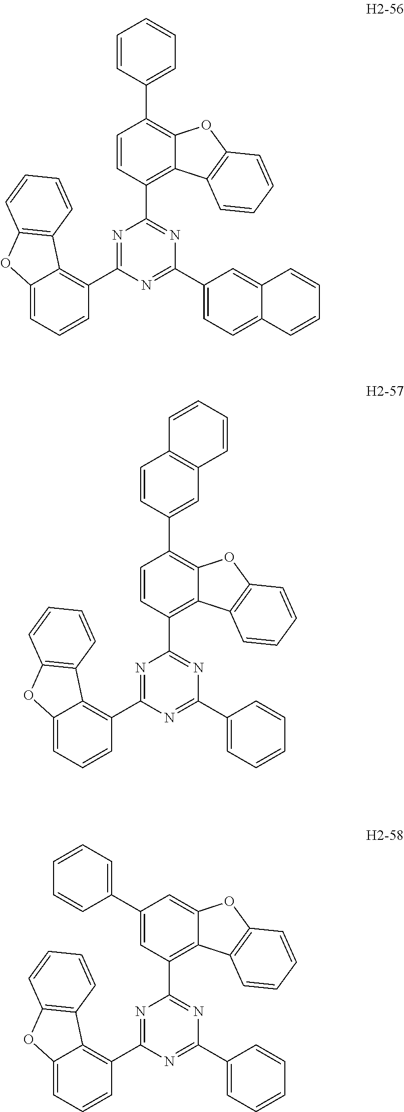 [SMILES: c1ccc(-c2ccc(-c3nc(-c4ccc5ccccc5c4)nc(-c4cccc5oc6ccccc6c45)n3)c3c2oc2ccccc23)cc1 ; c1ccc(-c2nc(-c3cccc4oc5ccccc5c34)nc(-c3ccc(-c4ccc5ccccc5c4)c4oc5ccccc5c34)n2)cc1 ; c1ccc(-c2cc(-c3nc(-c4ccccc4)nc(-c4cccc5oc6ccccc6c45)n3)c3c(c2)oc2ccccc23)cc1]

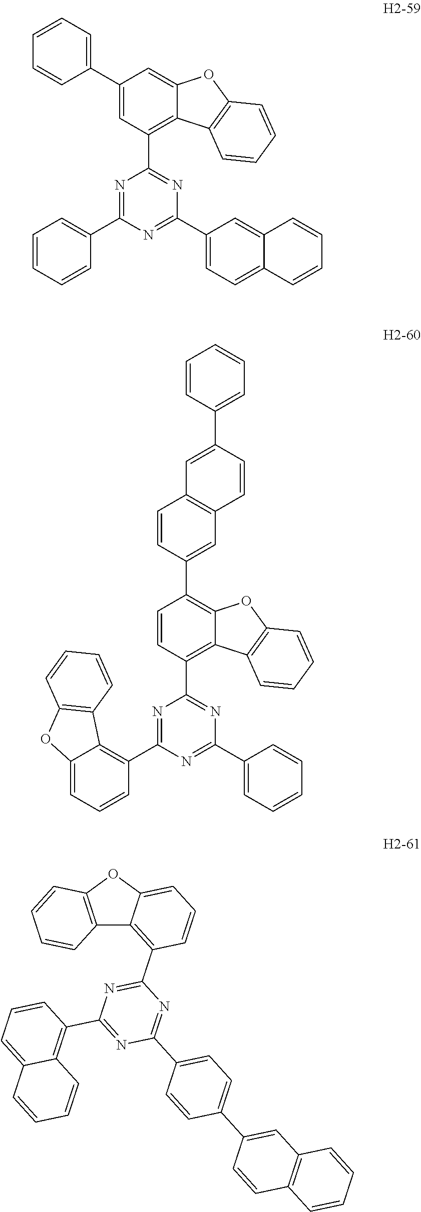 [SMILES: c1ccc(-c2cc(-c3nc(-c4ccccc4)nc(-c4ccc5ccccc5c4)n3)c3c(c2)oc2ccccc23)cc1 ; c1ccc(-c2ccc3cc(-c4ccc(-c5nc(-c6ccccc6)nc(-c6cccc7oc8ccccc8c67)n5)c5c4oc4ccccc45)ccc3c2)cc1 ; c1ccc2cc(-c3ccc(-c4nc(-c5cccc6ccccc56)nc(-c5cccc6oc7ccccc7c56)n4)cc3)ccc2c1]

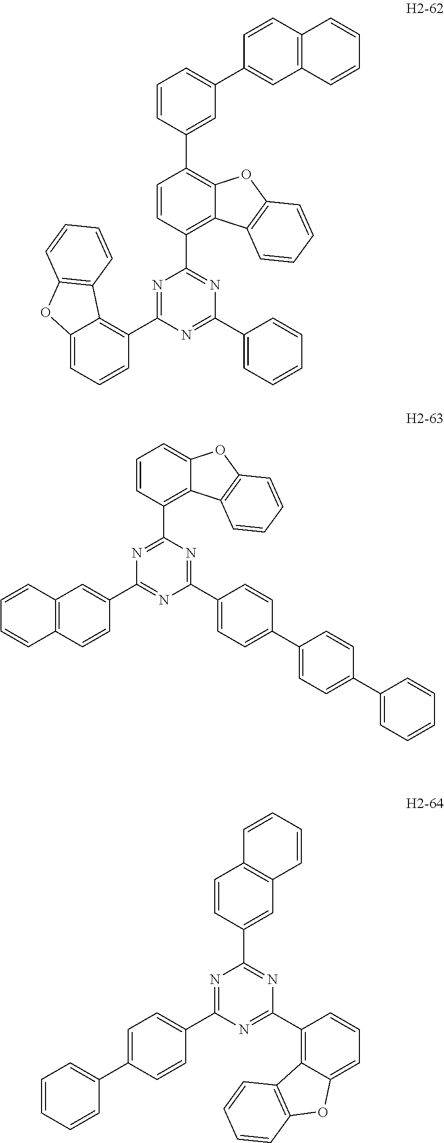 [SMILES: c1ccc(-c2nc(-c3cccc4oc5ccccc5c34)nc(-c3ccc(-c4cccc(-c5ccc6ccccc6c5)c4)c4oc5ccccc5c34)n2)cc1 ; c1ccc(-c2ccc(-c3ccc(-c4nc(-c5ccc6ccccc6c5)nc(-c5cccc6oc7ccccc7c56)n4)cc3)cc2)cc1 ; c1ccc(-c2ccc(-c3nc(-c4ccc5ccccc5c4)nc(-c4cccc5oc6ccccc6c45)n3)cc2)cc1]

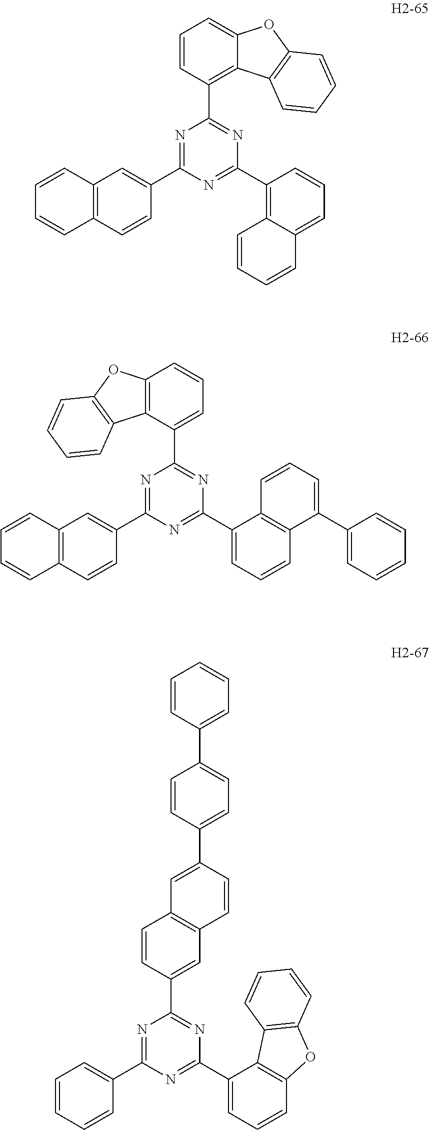 [SMILES: c1ccc2cc(-c3nc(-c4cccc5ccccc45)nc(-c4cccc5oc6ccccc6c45)n3)ccc2c1 ; c1ccc(-c2cccc3c(-c4nc(-c5ccc6ccccc6c5)nc(-c5cccc6oc7ccccc7c56)n4)cccc23)cc1 ; c1ccc(-c2ccc(-c3ccc4cc(-c5nc(-c6ccccc6)nc(-c6cccc7oc8ccccc8c67)n5)ccc4c3)cc2)cc1]

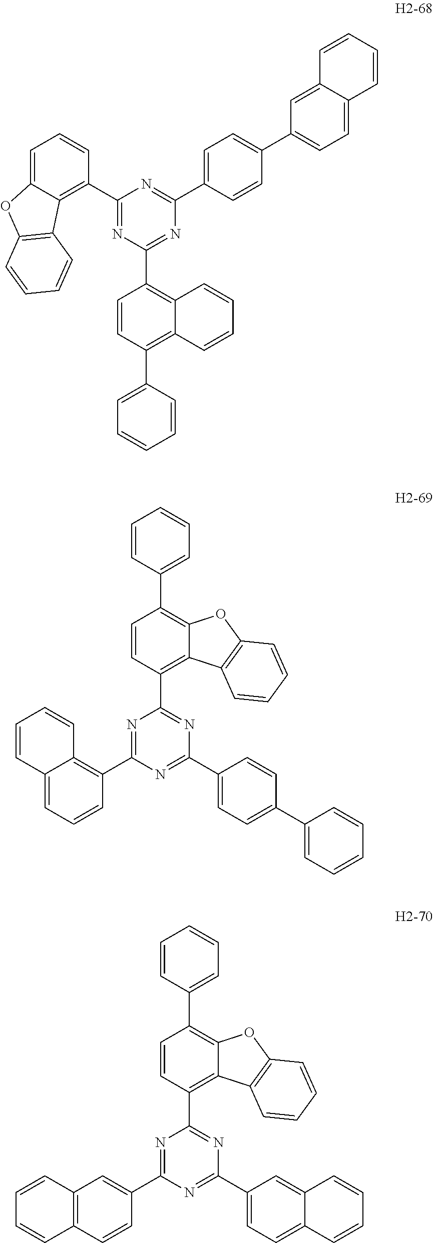 [SMILES: c1ccc(-c2ccc(-c3nc(-c4ccc(-c5ccc6ccccc6c5)cc4)nc(-c4cccc5oc6ccccc6c45)n3)c3ccccc23)cc1 ; c1ccc(-c2ccc(-c3nc(-c4cccc5ccccc45)nc(-c4ccc(-c5ccccc5)c5oc6ccccc6c45)n3)cc2)cc1 ; c1ccc(-c2ccc(-c3nc(-c4ccc5ccccc5c4)nc(-c4ccc5ccccc5c4)n3)c3c2oc2ccccc23)cc1]

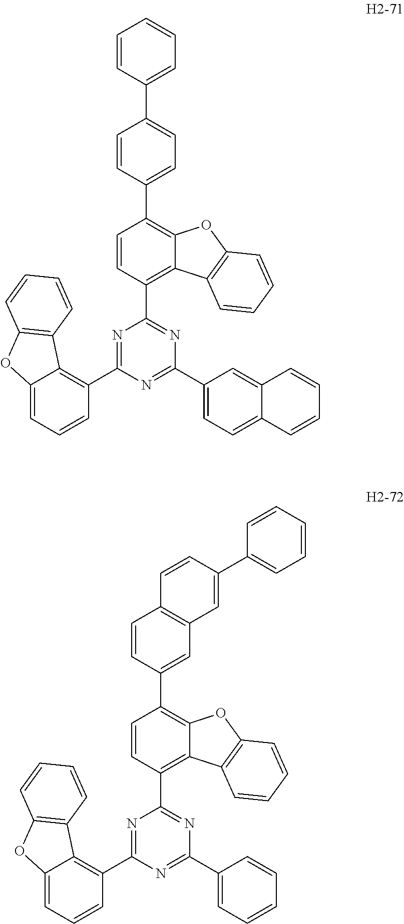 [SMILES: c1ccc(-c2ccc(-c3ccc(-c4nc(-c5ccc6ccccc6c5)nc(-c5cccc6oc7ccccc7c56)n4)c4c3oc3ccccc34)cc2)cc1 ; c1ccc(-c2ccc3ccc(-c4ccc(-c5nc(-c6ccccc6)nc(-c6cccc7oc8ccccc8c67)n5)c5c4oc4ccccc45)cc3c2)cc1]

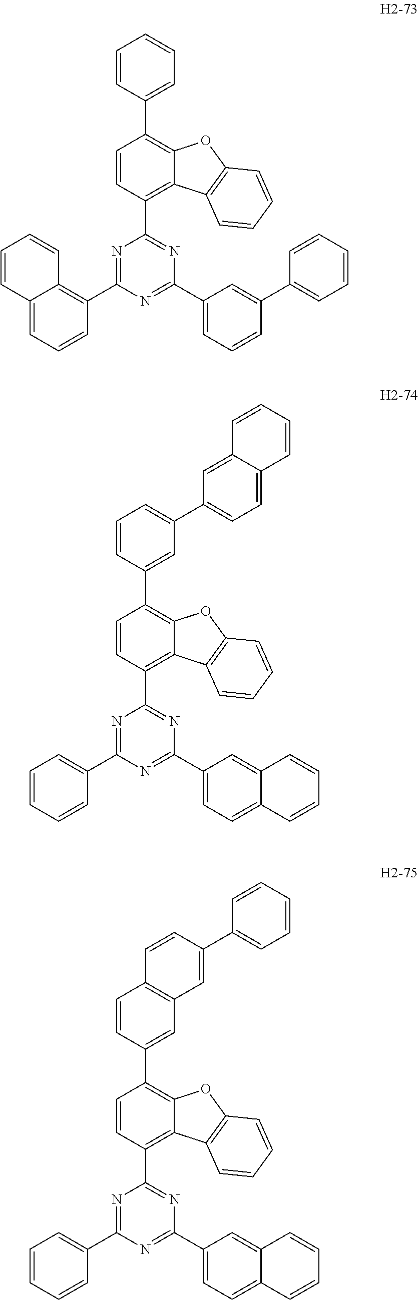 [SMILES: c1ccc(-c2cccc(-c3nc(-c4cccc5ccccc45)nc(-c4ccc(-c5ccccc5)c5oc6ccccc6c45)n3)c2)cc1 ; c1ccc(-c2nc(-c3ccc4ccccc4c3)nc(-c3ccc(-c4cccc(-c5ccc6ccccc6c5)c4)c4oc5ccccc5c34)n2)cc1 ; c1ccc(-c2ccc3ccc(-c4ccc(-c5nc(-c6ccccc6)nc(-c6ccc7ccccc7c6)n5)c5c4oc4ccccc45)cc3c2)cc1]

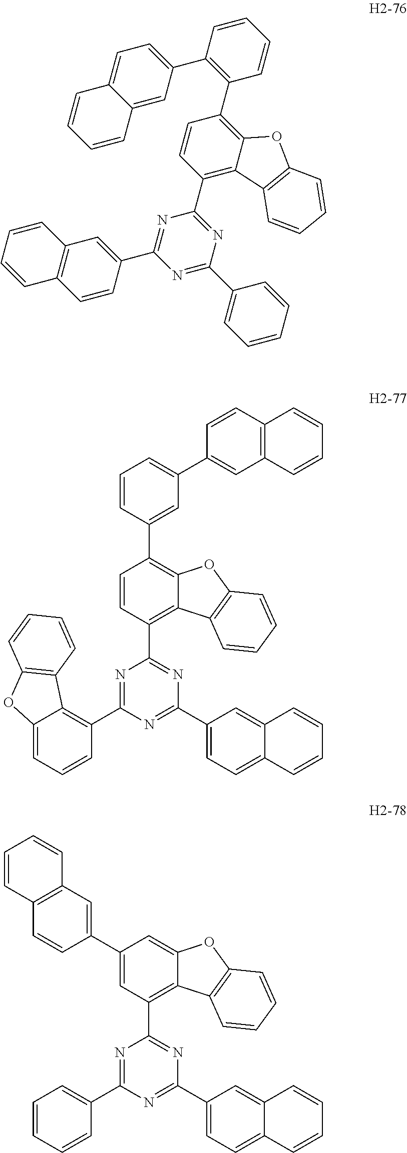 [SMILES: c1ccc(-c2nc(-c3ccc4ccccc4c3)nc(-c3ccc(-c4ccccc4-c4ccc5ccccc5c4)c4oc5ccccc5c34)n2)cc1 ; c1cc(-c2ccc3ccccc3c2)cc(-c2ccc(-c3nc(-c4ccc5ccccc5c4)nc(-c4cccc5oc6ccccc6c45)n3)c3c2oc2ccccc23)c1 ; c1ccc(-c2nc(-c3ccc4ccccc4c3)nc(-c3cc(-c4ccc5ccccc5c4)cc4oc5ccccc5c34)n2)cc1]

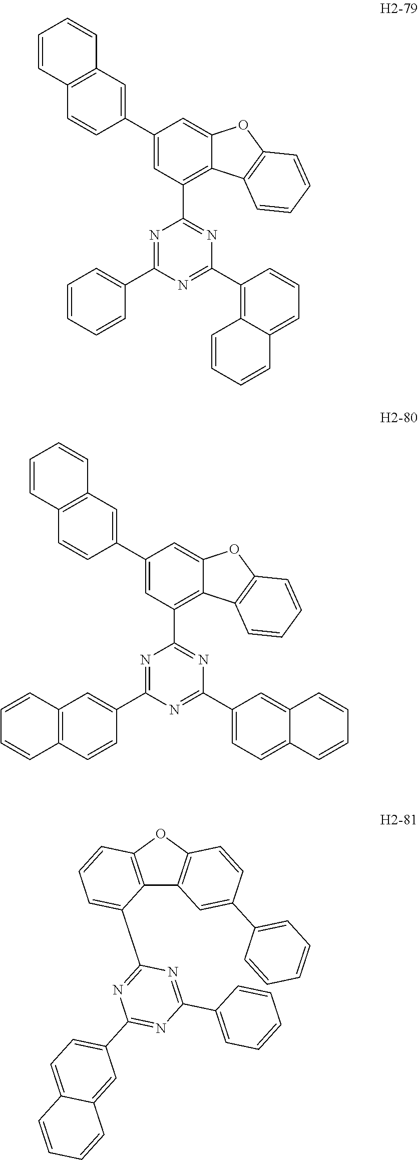 [SMILES: c1ccc(-c2nc(-c3cccc4ccccc34)nc(-c3cc(-c4ccc5ccccc5c4)cc4oc5ccccc5c34)n2)cc1 ; c1ccc2cc(-c3cc(-c4nc(-c5ccc6ccccc6c5)nc(-c5ccc6ccccc6c5)n4)c4c(c3)oc3ccccc34)ccc2c1 ; c1ccc(-c2ccc3oc4cccc(-c5nc(-c6ccccc6)nc(-c6ccc7ccccc7c6)n5)c4c3c2)cc1]

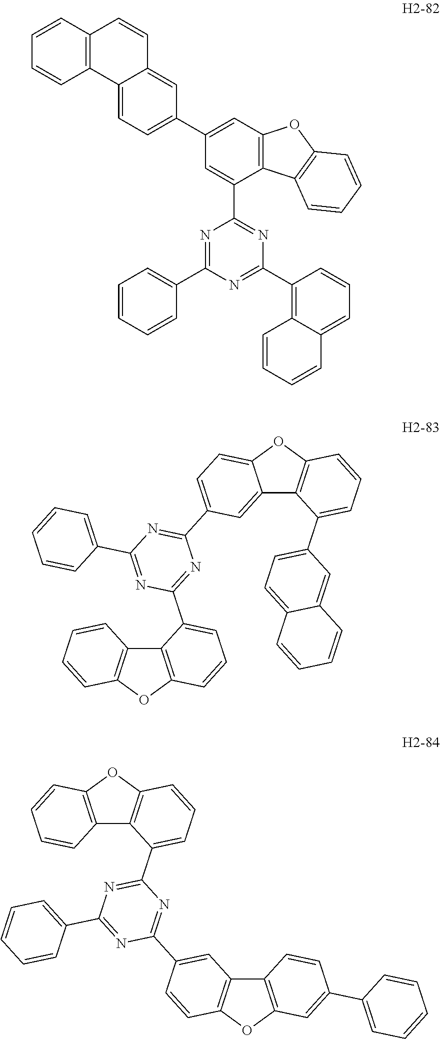 [SMILES: c1ccc(-c2nc(-c3cccc4ccccc34)nc(-c3cc(-c4ccc5c(ccc6ccccc65)c4)cc4oc5ccccc5c34)n2)cc1 ; c1ccc(-c2nc(-c3ccc4oc5cccc(-c6ccc7ccccc7c6)c5c4c3)nc(-c3cccc4oc5ccccc5c34)n2)cc1 ; c1ccc(-c2ccc3c(c2)oc2ccc(-c4nc(-c5ccccc5)nc(-c5cccc6oc7ccccc7c56)n4)cc23)cc1]

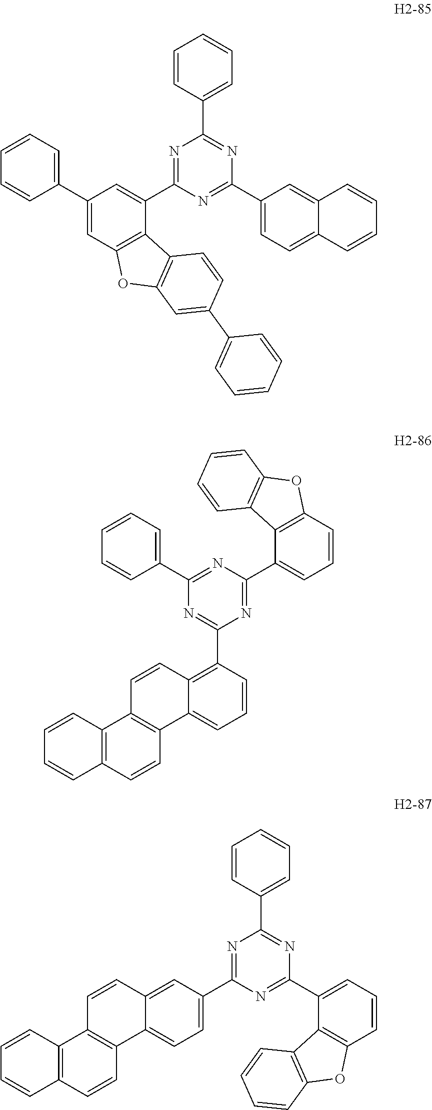 [SMILES: c1ccc(-c2ccc3c(c2)oc2cc(-c4ccccc4)cc(-c4nc(-c5ccccc5)nc(-c5ccc6ccccc6c5)n4)c23)cc1 ; c1ccc(-c2nc(-c3cccc4c3ccc3c5ccccc5ccc43)nc(-c3cccc4oc5ccccc5c34)n2)cc1 ; c1ccc(-c2nc(-c3ccc4c(ccc5c6ccccc6ccc45)c3)nc(-c3cccc4oc5ccccc5c34)n2)cc1]

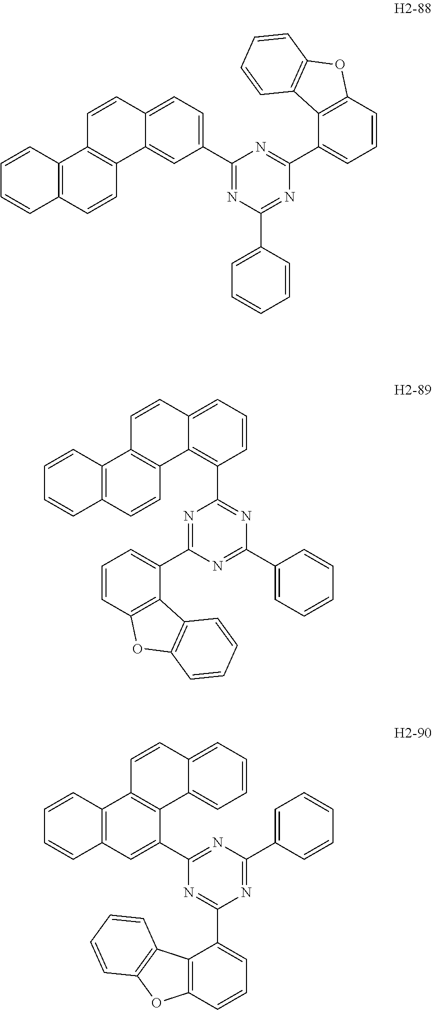 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3cccc4ccc5c6ccccc6ccc5c34)nc(-c3cccc4oc5ccccc5c34)n2)cc1 ; c1ccc(-c2nc(-c3cccc4oc5ccccc5c34)nc(-c3cc4ccccc4c4ccc5ccccc5c34)n2)cc1]

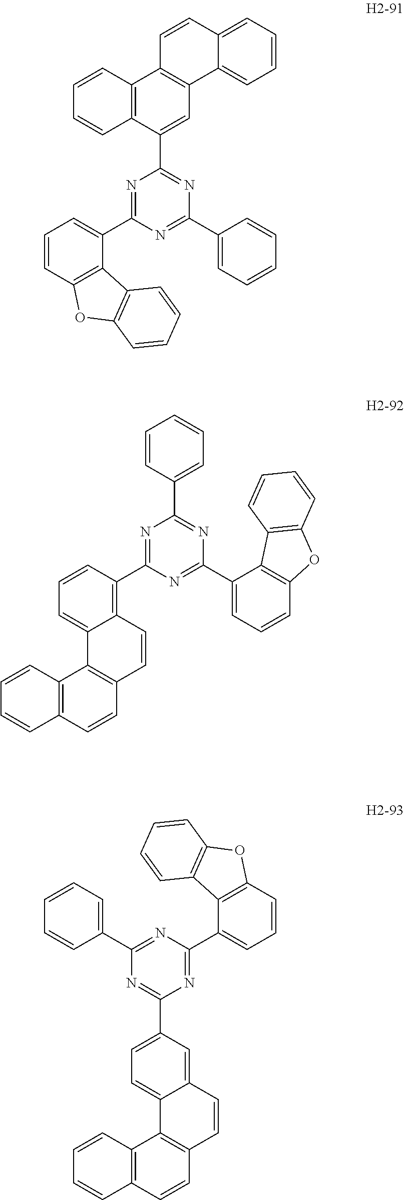 [SMILES: c1ccc(-c2nc(-c3cc4c5ccccc5ccc4c4ccccc34)nc(-c3cccc4oc5ccccc5c34)n2)cc1 ; c1ccc(-c2nc(-c3cccc4c3ccc3ccc5ccccc5c34)nc(-c3cccc4oc5ccccc5c34)n2)cc1 ; c1ccc(-c2nc(-c3ccc4c(ccc5ccc6ccccc6c54)c3)nc(-c3cccc4oc5ccccc5c34)n2)cc1]

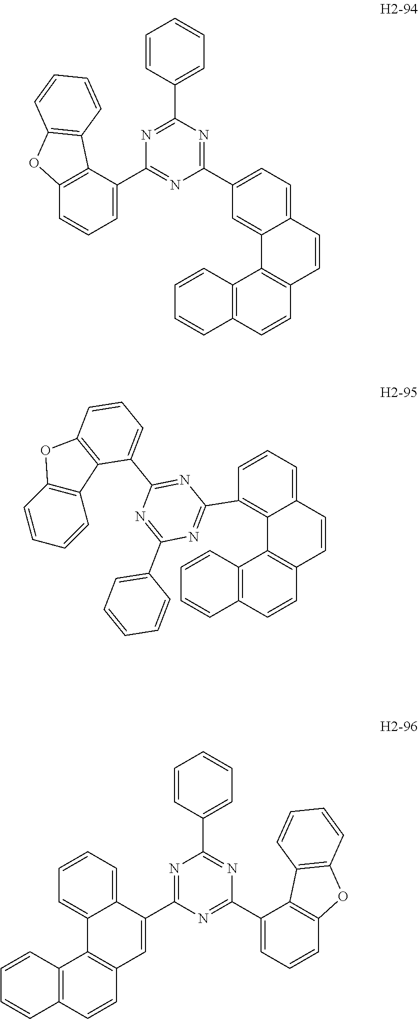 [SMILES: c1ccc(-c2nc(-c3ccc4ccc5ccc6ccccc6c5c4c3)nc(-c3cccc4oc5ccccc5c34)n2)cc1 ; c1ccc(-c2nc(-c3cccc4oc5ccccc5c34)nc(-c3cccc4ccc5ccc6ccccc6c5c34)n2)cc1 ; c1ccc(-c2nc(-c3cc4ccc5ccccc5c4c4ccccc34)nc(-c3cccc4oc5ccccc5c34)n2)cc1]

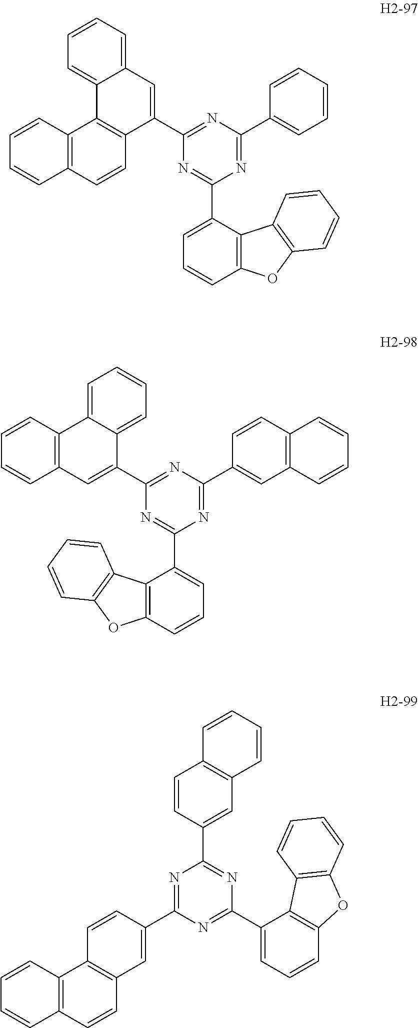 [SMILES: c1ccc(-c2nc(-c3cc4ccccc4c4c3ccc3ccccc34)nc(-c3cccc4oc5ccccc5c34)n2)cc1 ; c1ccc2cc(-c3nc(-c4cc5ccccc5c5ccccc45)nc(-c4cccc5oc6ccccc6c45)n3)ccc2c1 ; c1ccc2cc(-c3nc(-c4ccc5c(ccc6ccccc65)c4)nc(-c4cccc5oc6ccccc6c45)n3)ccc2c1]

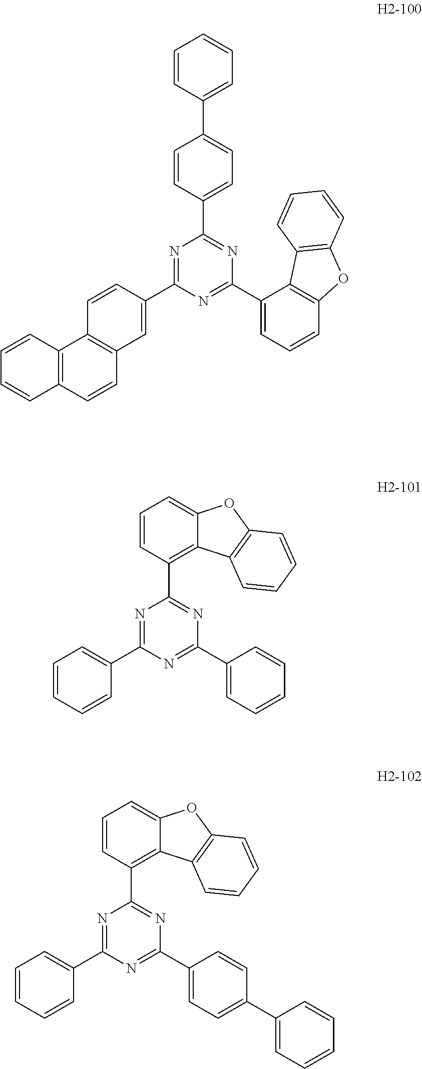 [SMILES: c1ccc(-c2ccc(-c3nc(-c4ccc5c(ccc6ccccc65)c4)nc(-c4cccc5oc6ccccc6c45)n3)cc2)cc1 ; c1ccc(-c2nc(-c3ccccc3)nc(-c3cccc4oc5ccccc5c34)n2)cc1 ; c1ccc(-c2ccc(-c3nc(-c4ccccc4)nc(-c4cccc5oc6ccccc6c45)n3)cc2)cc1]

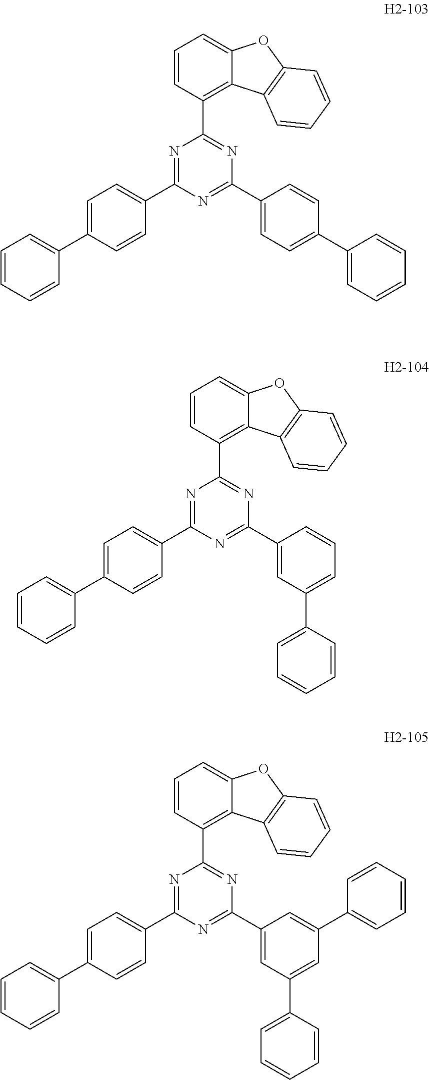 [SMILES: c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4cccc(-c5ccccc5)c4)nc(-c4cccc5oc6ccccc6c45)n3)cc2)cc1 ; c1ccc(-c2ccc(-c3nc(-c4cc(-c5ccccc5)cc(-c5ccccc5)c4)nc(-c4cccc5oc6ccccc6c45)n3)cc2)cc1]

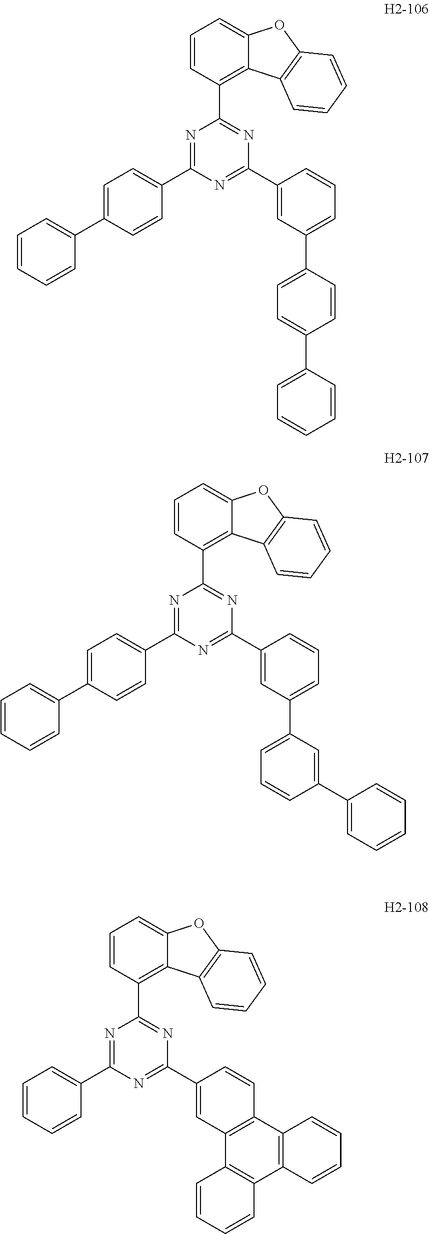 [SMILES: c1ccc(-c2ccc(-c3cccc(-c4nc(-c5ccc(-c6ccccc6)cc5)nc(-c5cccc6oc7ccccc7c56)n4)c3)cc2)cc1 ; c1ccc(-c2ccc(-c3nc(-c4cccc(-c5cccc(-c6ccccc6)c5)c4)nc(-c4cccc5oc6ccccc6c45)n3)cc2)cc1 ; c1ccc(-c2nc(-c3ccc4c5ccccc5c5ccccc5c4c3)nc(-c3cccc4oc5ccccc5c34)n2)cc1]

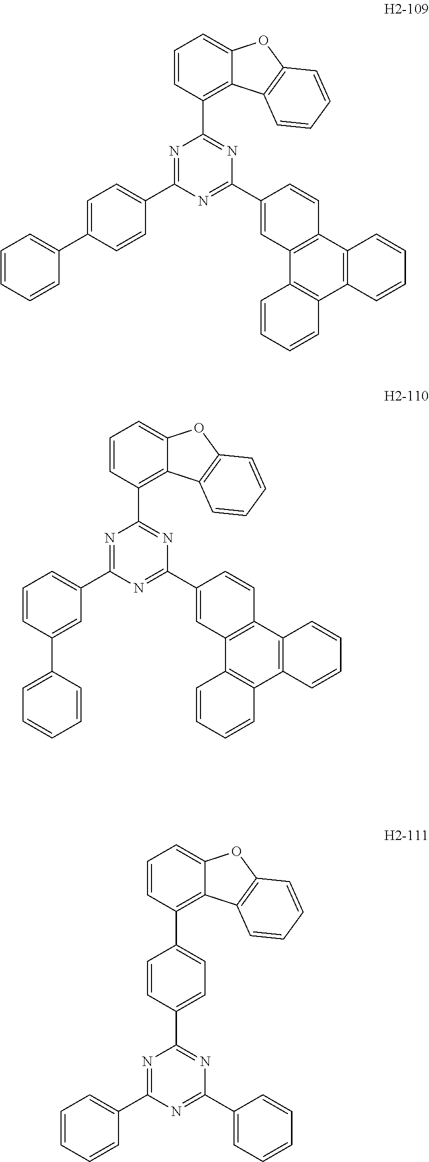 [SMILES: c1ccc(-c2ccc(-c3nc(-c4ccc5c6ccccc6c6ccccc6c5c4)nc(-c4cccc5oc6ccccc6c45)n3)cc2)cc1 ; c1ccc(-c2cccc(-c3nc(-c4ccc5c6ccccc6c6ccccc6c5c4)nc(-c4cccc5oc6ccccc6c45)n3)c2)cc1 ; c1ccc(-c2nc(-c3ccccc3)nc(-c3ccc(-c4cccc5oc6ccccc6c45)cc3)n2)cc1]

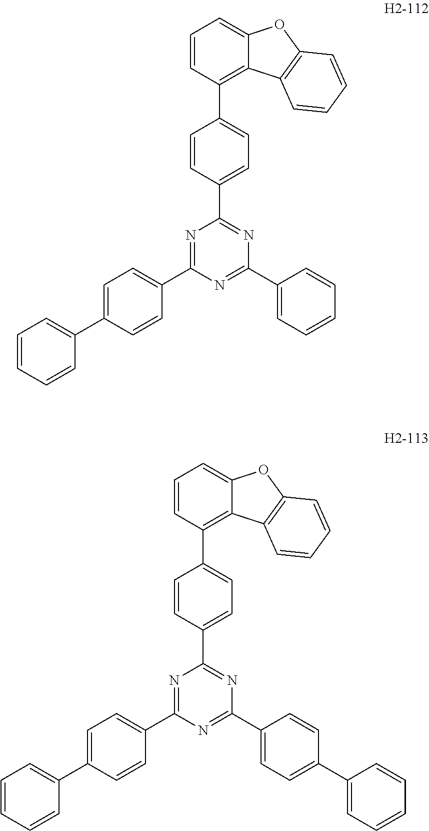 [SMILES: c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccc(-c5ccccc5)cc4)nc(-c4ccc(-c5cccc6oc7ccccc7c56)cc4)n3)cc2)cc1]

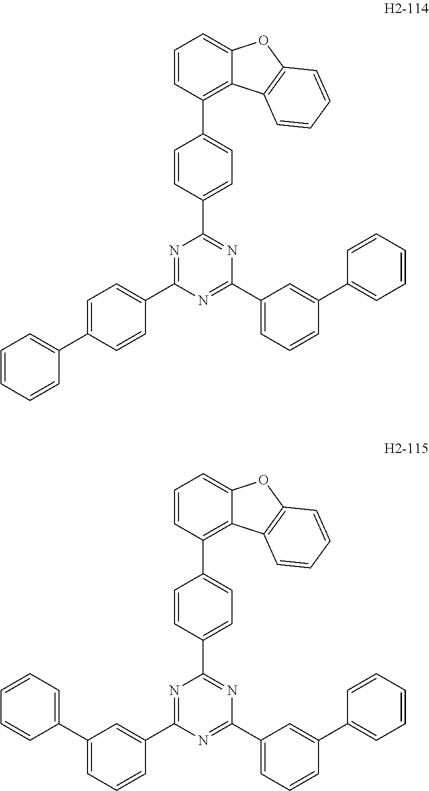 [SMILES: c1ccc(-c2ccc(-c3nc(-c4ccc(-c5cccc6oc7ccccc7c56)cc4)nc(-c4cccc(-c5ccccc5)c4)n3)cc2)cc1 ; c1ccc(-c2cccc(-c3nc(-c4ccc(-c5cccc6oc7ccccc7c56)cc4)nc(-c4cccc(-c5ccccc5)c4)n3)c2)cc1]

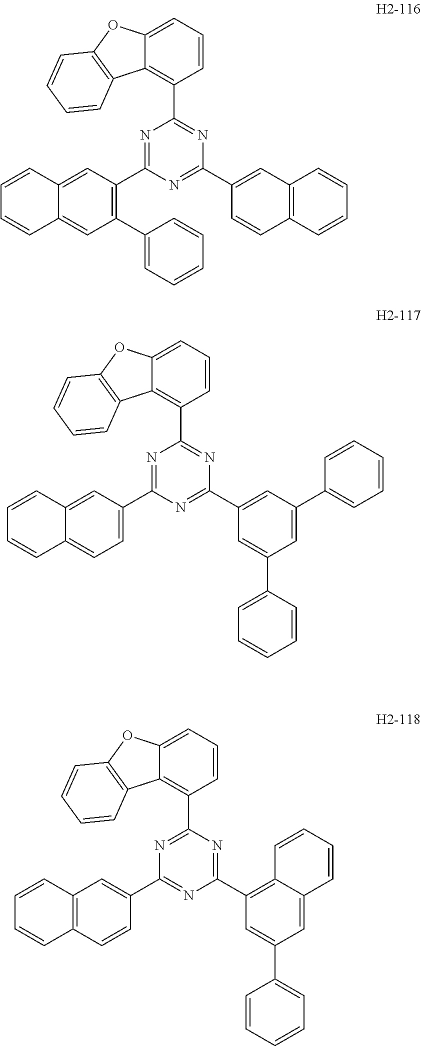 [SMILES: c1ccc(-c2cc3ccccc3cc2-c2nc(-c3ccc4ccccc4c3)nc(-c3cccc4oc5ccccc5c34)n2)cc1 ; c1ccc(-c2cc(-c3ccccc3)cc(-c3nc(-c4ccc5ccccc5c4)nc(-c4cccc5oc6ccccc6c45)n3)c2)cc1 ; c1ccc(-c2cc(-c3nc(-c4ccc5ccccc5c4)nc(-c4cccc5oc6ccccc6c45)n3)c3ccccc3c2)cc1]

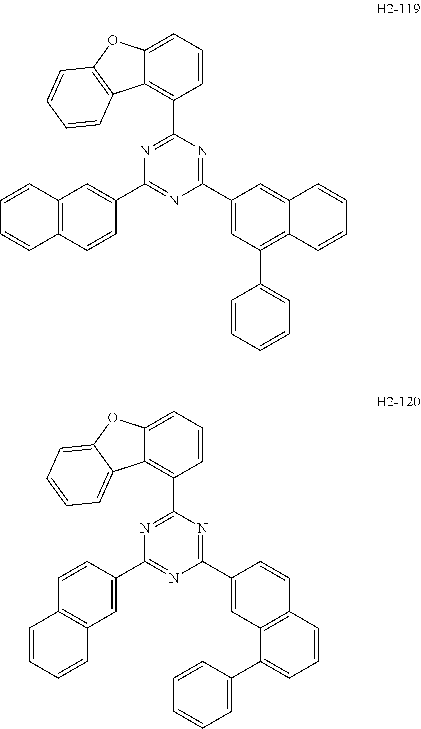 [SMILES: c1ccc(-c2cc(-c3nc(-c4ccc5ccccc5c4)nc(-c4cccc5oc6ccccc6c45)n3)cc3ccccc23)cc1 ; c1ccc(-c2cccc3ccc(-c4nc(-c5ccc6ccccc6c5)nc(-c5cccc6oc7ccccc7c56)n4)cc23)cc1]

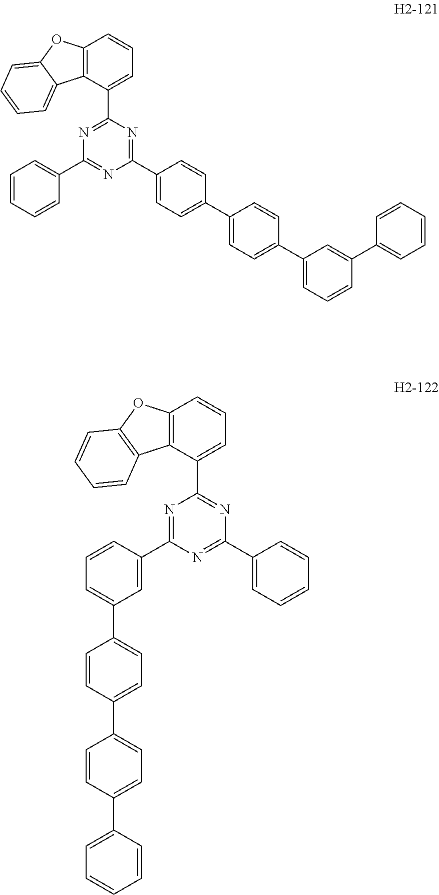 [SMILES: c1ccc(-c2cccc(-c3ccc(-c4ccc(-c5nc(-c6ccccc6)nc(-c6cccc7oc8ccccc8c67)n5)cc4)cc3)c2)cc1 ; c1ccc(-c2ccc(-c3ccc(-c4cccc(-c5nc(-c6ccccc6)nc(-c6cccc7oc8ccccc8c67)n5)c4)cc3)cc2)cc1]

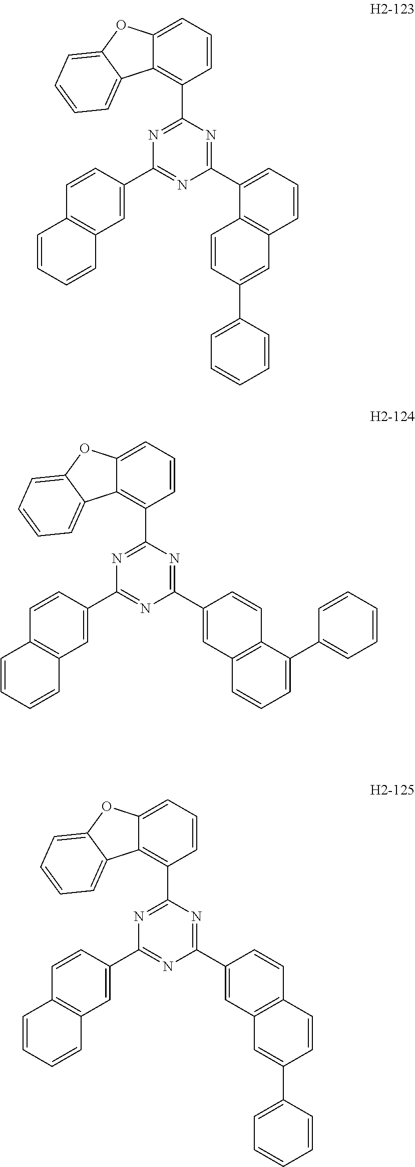 [SMILES: c1ccc(-c2ccc3c(-c4nc(-c5ccc6ccccc6c5)nc(-c5cccc6oc7ccccc7c56)n4)cccc3c2)cc1 ; c1ccc(-c2cccc3cc(-c4nc(-c5ccc6ccccc6c5)nc(-c5cccc6oc7ccccc7c56)n4)ccc23)cc1 ; c1ccc(-c2ccc3ccc(-c4nc(-c5ccc6ccccc6c5)nc(-c5cccc6oc7ccccc7c56)n4)cc3c2)cc1]

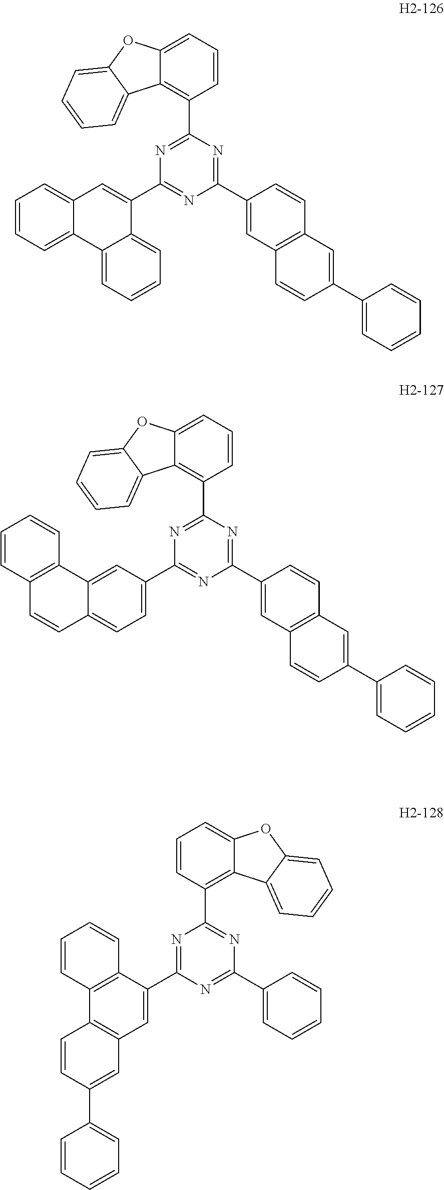 [SMILES: c1ccc(-c2ccc3cc(-c4nc(-c5cc6ccccc6c6ccccc56)nc(-c5cccc6oc7ccccc7c56)n4)ccc3c2)cc1 ; c1ccc(-c2ccc3cc(-c4nc(-c5ccc6ccc7ccccc7c6c5)nc(-c5cccc6oc7ccccc7c56)n4)ccc3c2)cc1 ; c1ccc(-c2ccc3c(c2)cc(-c2nc(-c4ccccc4)nc(-c4cccc5oc6ccccc6c45)n2)c2ccccc23)cc1]

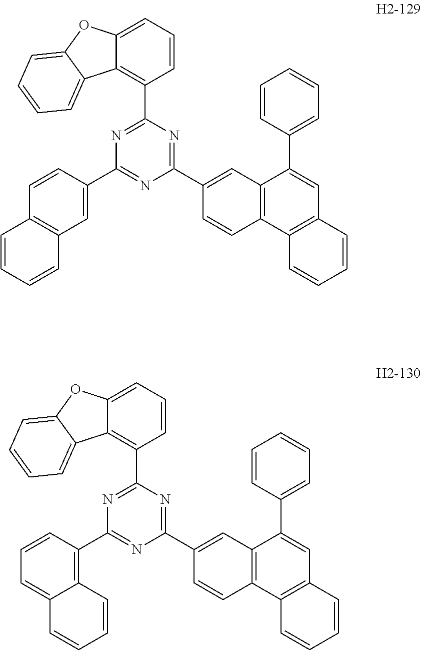 [SMILES: c1ccc(-c2cc3ccccc3c3ccc(-c4nc(-c5ccc6ccccc6c5)nc(-c5cccc6oc7ccccc7c56)n4)cc23)cc1 ; c1ccc(-c2cc3ccccc3c3ccc(-c4nc(-c5cccc6ccccc56)nc(-c5cccc6oc7ccccc7c56)n4)cc23)cc1]

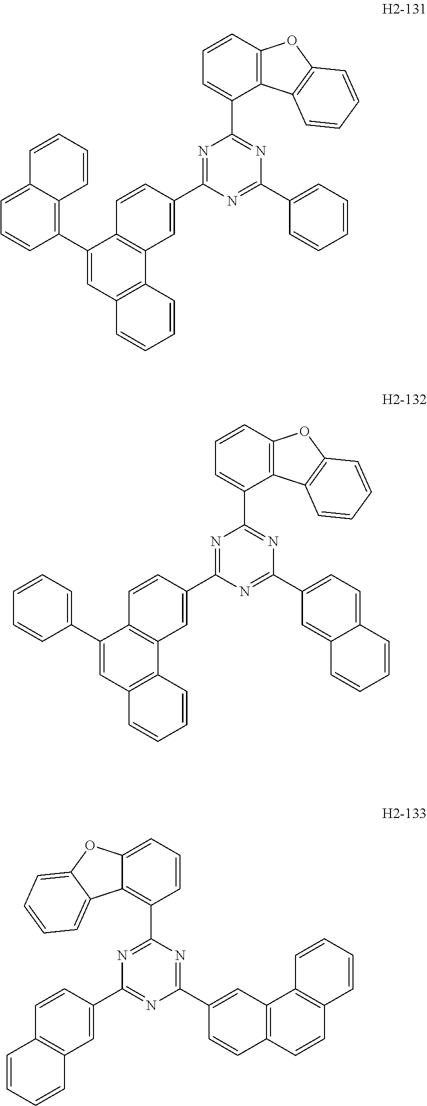 [SMILES: c1ccc(-c2nc(-c3ccc4c(-c5cccc6ccccc56)cc5ccccc5c4c3)nc(-c3cccc4oc5ccccc5c34)n2)cc1 ; c1ccc(-c2cc3ccccc3c3cc(-c4nc(-c5ccc6ccccc6c5)nc(-c5cccc6oc7ccccc7c56)n4)ccc23)cc1 ; c1ccc2cc(-c3nc(-c4ccc5ccc6ccccc6c5c4)nc(-c4cccc5oc6ccccc6c45)n3)ccc2c1]

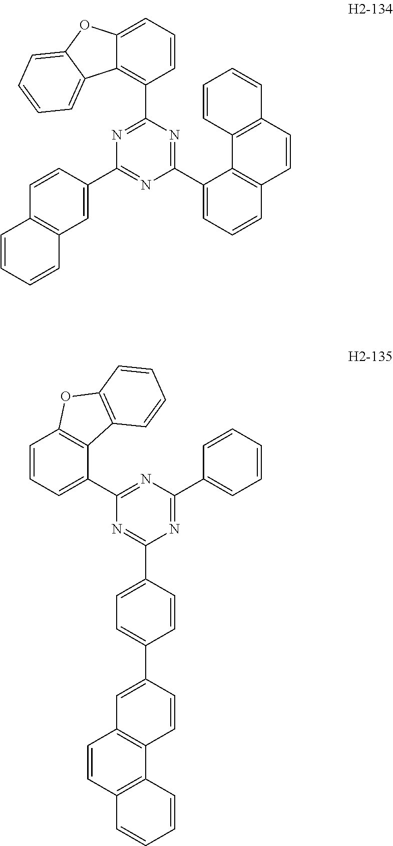 [SMILES: c1ccc2cc(-c3nc(-c4cccc5ccc6ccccc6c45)nc(-c4cccc5oc6ccccc6c45)n3)ccc2c1 ; c1ccc(-c2nc(-c3ccc(-c4ccc5c(ccc6ccccc65)c4)cc3)nc(-c3cccc4oc5ccccc5c34)n2)cc1]

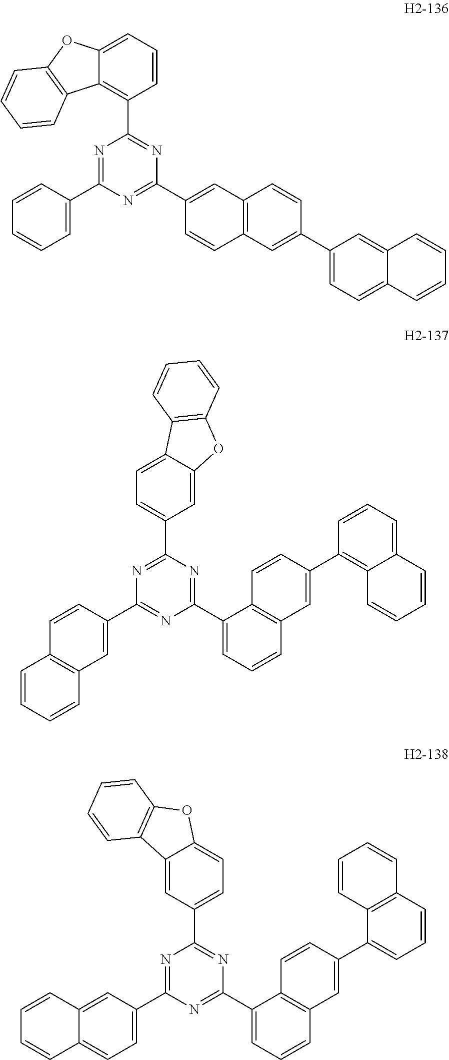 [SMILES: c1ccc(-c2nc(-c3ccc4cc(-c5ccc6ccccc6c5)ccc4c3)nc(-c3cccc4oc5ccccc5c34)n2)cc1 ; c1ccc2cc(-c3nc(-c4ccc5c(c4)oc4ccccc45)nc(-c4cccc5cc(-c6cccc7ccccc67)ccc45)n3)ccc2c1 ; c1ccc2cc(-c3nc(-c4ccc5oc6ccccc6c5c4)nc(-c4cccc5cc(-c6cccc7ccccc67)ccc45)n3)ccc2c1]

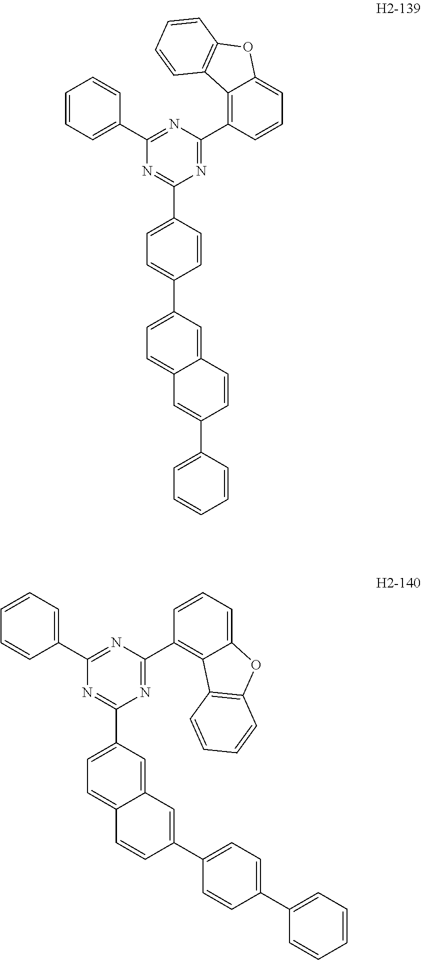 [SMILES: c1ccc(-c2ccc3cc(-c4ccc(-c5nc(-c6ccccc6)nc(-c6cccc7oc8ccccc8c67)n5)cc4)ccc3c2)cc1 ; c1ccc(-c2ccc(-c3ccc4ccc(-c5nc(-c6ccccc6)nc(-c6cccc7oc8ccccc8c67)n5)cc4c3)cc2)cc1]

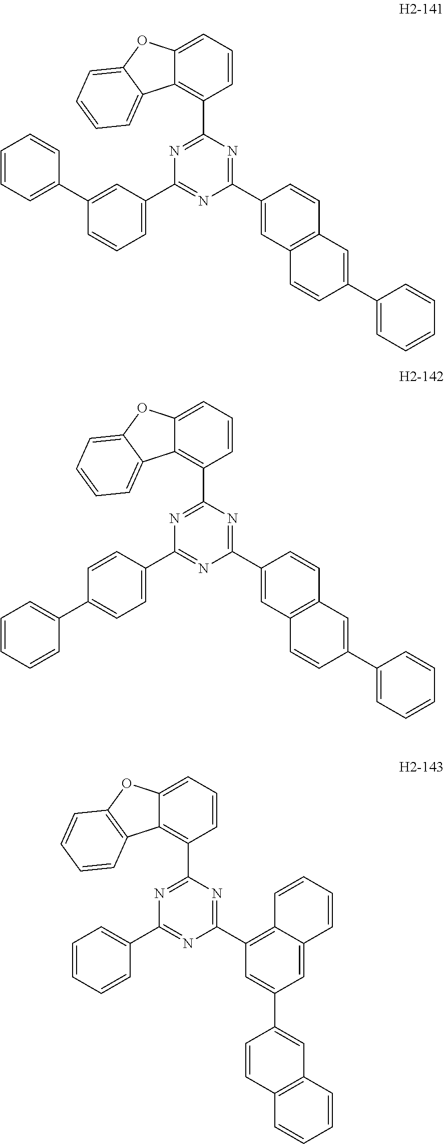 [SMILES: c1ccc(-c2cccc(-c3nc(-c4ccc5cc(-c6ccccc6)ccc5c4)nc(-c4cccc5oc6ccccc6c45)n3)c2)cc1 ; c1ccc(-c2ccc(-c3nc(-c4ccc5cc(-c6ccccc6)ccc5c4)nc(-c4cccc5oc6ccccc6c45)n3)cc2)cc1 ; c1ccc(-c2nc(-c3cc(-c4ccc5ccccc5c4)cc4ccccc34)nc(-c3cccc4oc5ccccc5c34)n2)cc1]

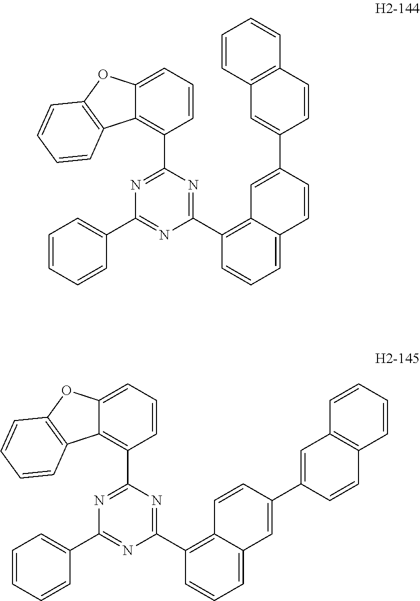 [SMILES: c1ccc(-c2nc(-c3cccc4ccc(-c5ccc6ccccc6c5)cc34)nc(-c3cccc4oc5ccccc5c34)n2)cc1 ; c1ccc(-c2nc(-c3cccc4cc(-c5ccc6ccccc6c5)ccc34)nc(-c3cccc4oc5ccccc5c34)n2)cc1]

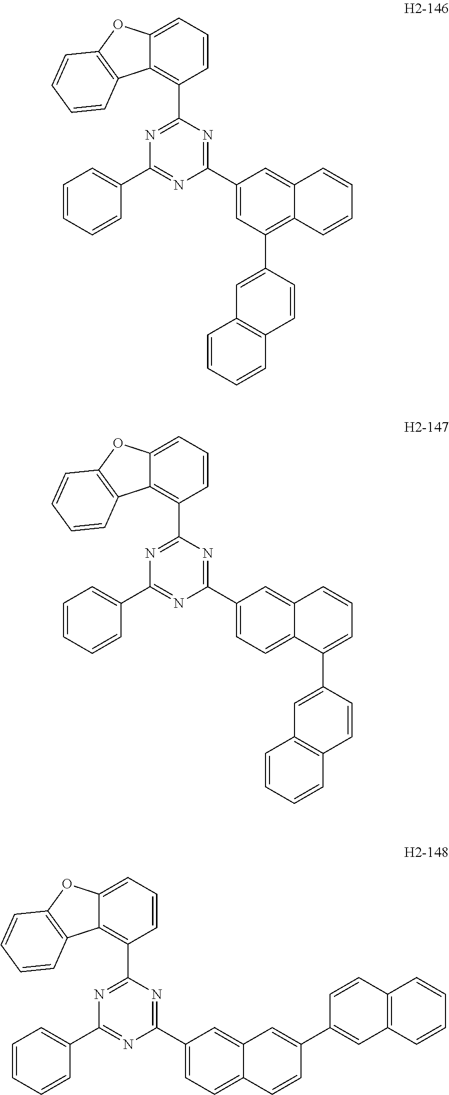 [SMILES: c1ccc(-c2nc(-c3cc(-c4ccc5ccccc5c4)c4ccccc4c3)nc(-c3cccc4oc5ccccc5c34)n2)cc1 ; c1ccc(-c2nc(-c3ccc4c(-c5ccc6ccccc6c5)cccc4c3)nc(-c3cccc4oc5ccccc5c34)n2)cc1 ; c1ccc(-c2nc(-c3ccc4ccc(-c5ccc6ccccc6c5)cc4c3)nc(-c3cccc4oc5ccccc5c34)n2)cc1]

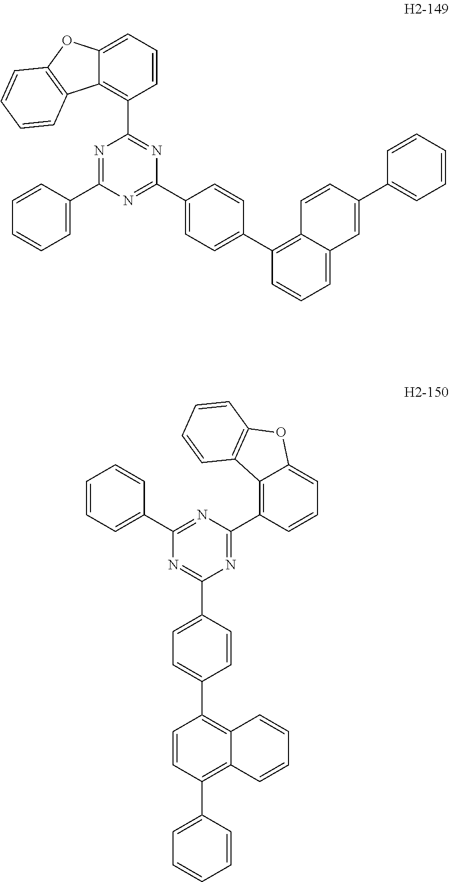 [SMILES: c1ccc(-c2ccc3c(-c4ccc(-c5nc(-c6ccccc6)nc(-c6cccc7oc8ccccc8c67)n5)cc4)cccc3c2)cc1 ; c1ccc(-c2nc(-c3ccc(-c4ccc(-c5ccccc5)c5ccccc45)cc3)nc(-c3cccc4oc5ccccc5c34)n2)cc1]

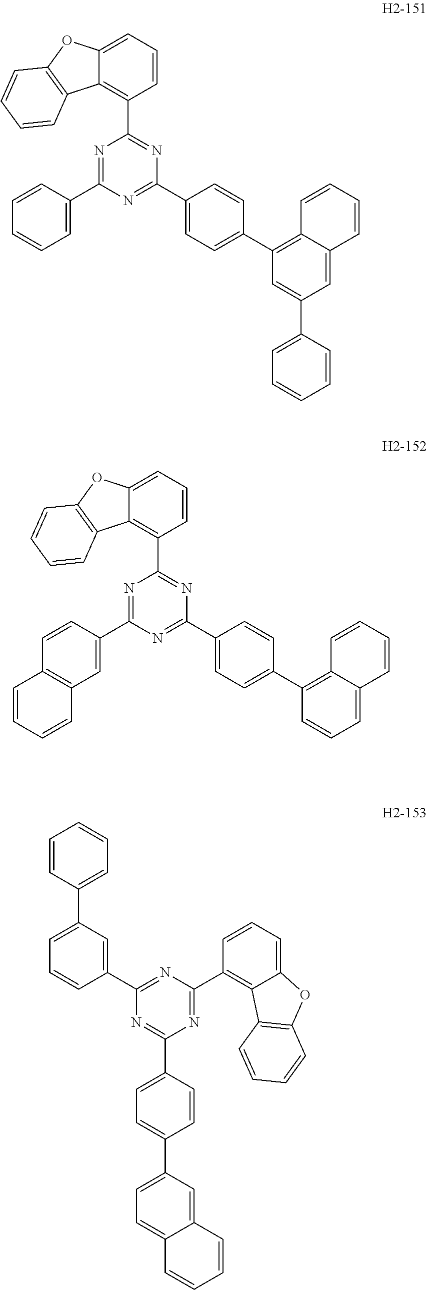 [SMILES: c1ccc(-c2cc(-c3ccc(-c4nc(-c5ccccc5)nc(-c5cccc6oc7ccccc7c56)n4)cc3)c3ccccc3c2)cc1 ; c1ccc2cc(-c3nc(-c4ccc(-c5cccc6ccccc56)cc4)nc(-c4cccc5oc6ccccc6c45)n3)ccc2c1 ; c1ccc(-c2cccc(-c3nc(-c4ccc(-c5ccc6ccccc6c5)cc4)nc(-c4cccc5oc6ccccc6c45)n3)c2)cc1]

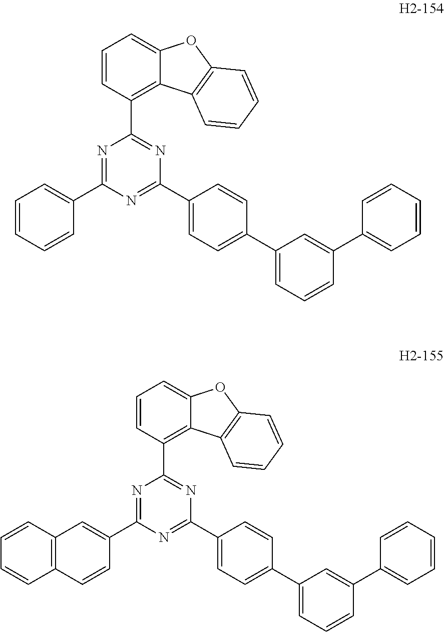 [SMILES: c1ccc(-c2cccc(-c3ccc(-c4nc(-c5ccccc5)nc(-c5cccc6oc7ccccc7c56)n4)cc3)c2)cc1 ; c1ccc(-c2cccc(-c3ccc(-c4nc(-c5ccc6ccccc6c5)nc(-c5cccc6oc7ccccc7c56)n4)cc3)c2)cc1]

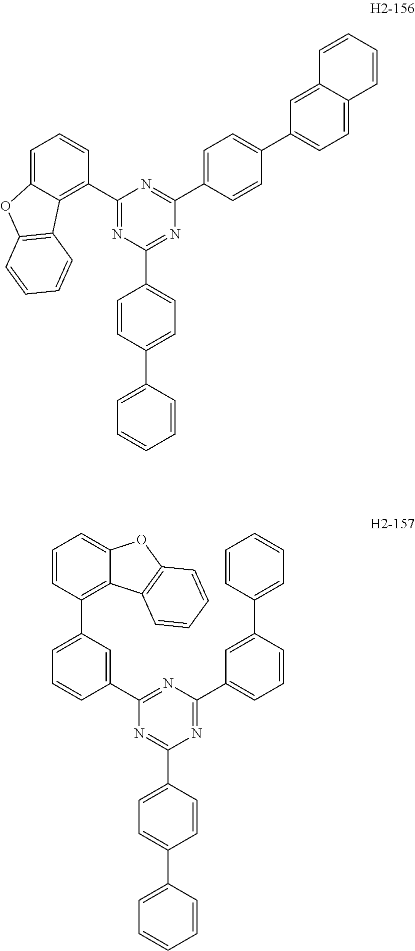 [SMILES: c1ccc(-c2ccc(-c3nc(-c4ccc(-c5ccc6ccccc6c5)cc4)nc(-c4cccc5oc6ccccc6c45)n3)cc2)cc1 ; c1ccc(-c2ccc(-c3nc(-c4cccc(-c5ccccc5)c4)nc(-c4cccc(-c5cccc6oc7ccccc7c56)c4)n3)cc2)cc1]

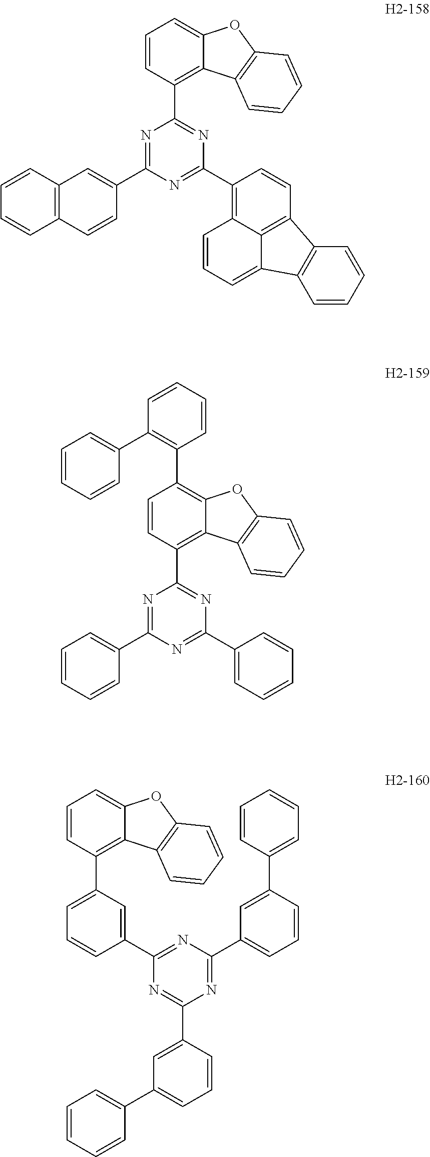 [SMILES: c1ccc2c(c1)-c1cccc3c(-c4nc(-c5ccc6ccccc6c5)nc(-c5cccc6oc7ccccc7c56)n4)ccc-2c13 ; c1ccc(-c2nc(-c3ccccc3)nc(-c3ccc(-c4ccccc4-c4ccccc4)c4oc5ccccc5c34)n2)cc1 ; c1ccc(-c2cccc(-c3nc(-c4cccc(-c5ccccc5)c4)nc(-c4cccc(-c5cccc6oc7ccccc7c56)c4)n3)c2)cc1]

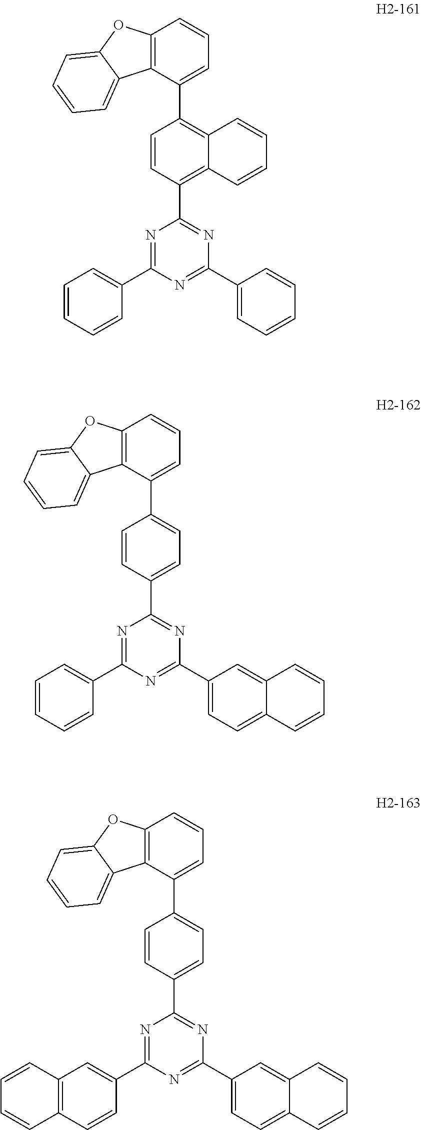 [SMILES: c1ccc(-c2nc(-c3ccccc3)nc(-c3ccc(-c4cccc5oc6ccccc6c45)c4ccccc34)n2)cc1 ; c1ccc(-c2nc(-c3ccc(-c4cccc5oc6ccccc6c45)cc3)nc(-c3ccc4ccccc4c3)n2)cc1 ; c1ccc2cc(-c3nc(-c4ccc(-c5cccc6oc7ccccc7c56)cc4)nc(-c4ccc5ccccc5c4)n3)ccc2c1]

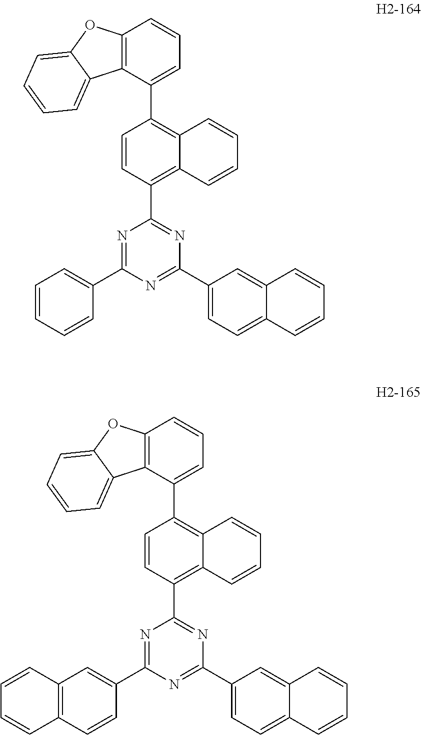 [SMILES: c1ccc(-c2nc(-c3ccc4ccccc4c3)nc(-c3ccc(-c4cccc5oc6ccccc6c45)c4ccccc34)n2)cc1 ; c1ccc2cc(-c3nc(-c4ccc5ccccc5c4)nc(-c4ccc(-c5cccc6oc7ccccc7c56)c5ccccc45)n3)ccc2c1]

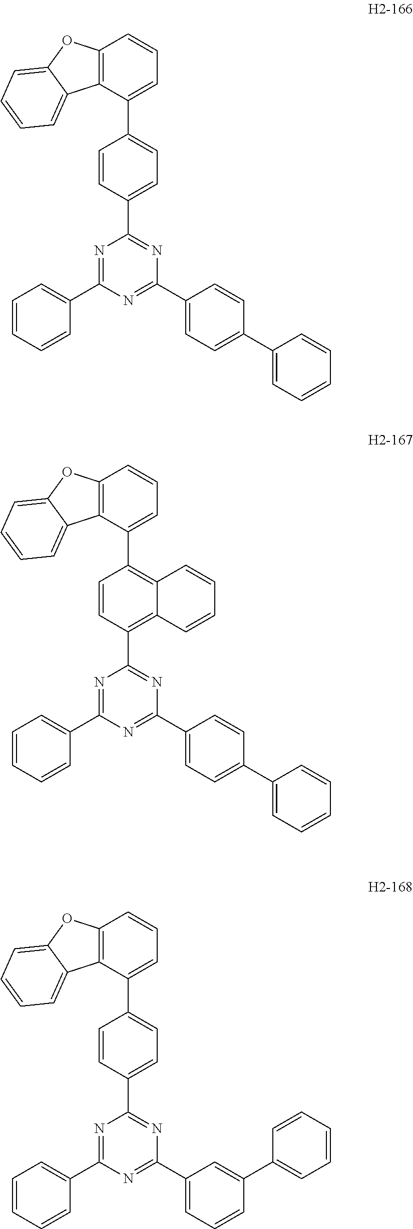 [SMILES: c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccccc4)nc(-c4ccc(-c5cccc6oc7ccccc7c56)c5ccccc45)n3)cc2)cc1 ; c1ccc(-c2cccc(-c3nc(-c4ccccc4)nc(-c4ccc(-c5cccc6oc7ccccc7c56)cc4)n3)c2)cc1]

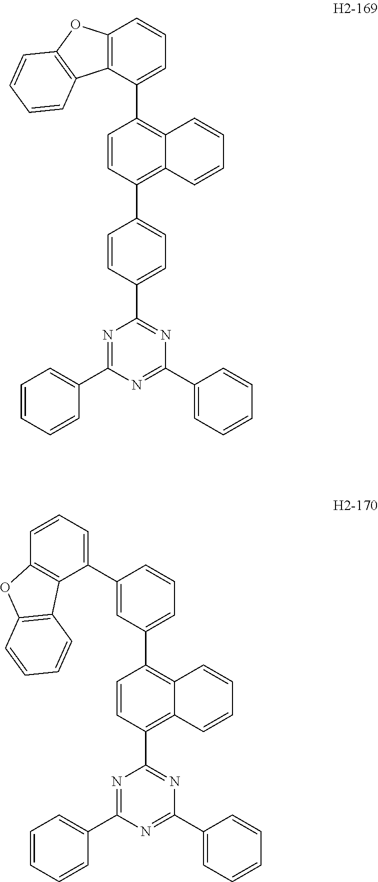 [SMILES: c1ccc(-c2nc(-c3ccccc3)nc(-c3ccc(-c4ccc(-c5cccc6oc7ccccc7c56)c5ccccc45)cc3)n2)cc1 ; c1ccc(-c2nc(-c3ccccc3)nc(-c3ccc(-c4cccc(-c5cccc6oc7ccccc7c56)c4)c4ccccc34)n2)cc1]

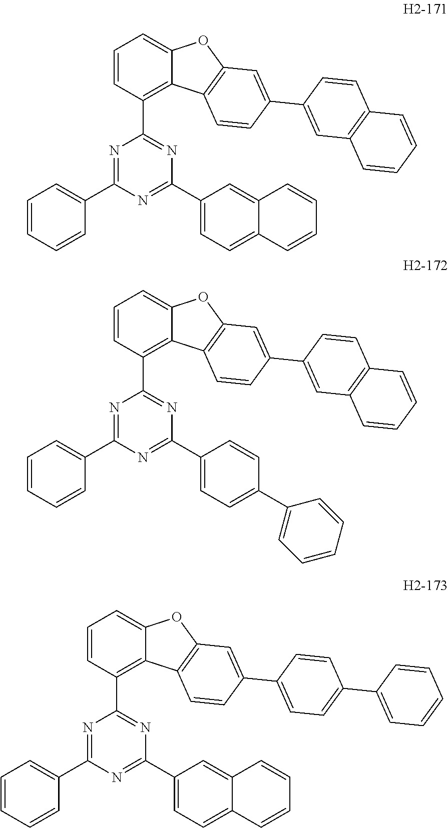 [SMILES: c1ccc(-c2nc(-c3ccc4ccccc4c3)nc(-c3cccc4oc5cc(-c6ccc7ccccc7c6)ccc5c34)n2)cc1 ; c1ccc(-c2ccc(-c3nc(-c4ccccc4)nc(-c4cccc5oc6cc(-c7ccc8ccccc8c7)ccc6c45)n3)cc2)cc1 ; c1ccc(-c2ccc(-c3ccc4c(c3)oc3cccc(-c5nc(-c6ccccc6)nc(-c6ccc7ccccc7c6)n5)c34)cc2)cc1]

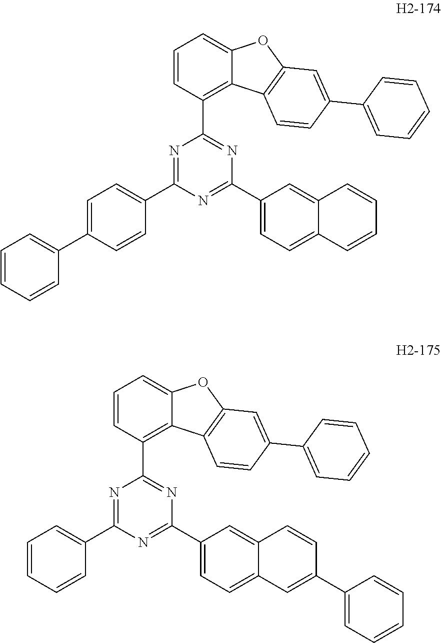 [SMILES: c1ccc(-c2ccc(-c3nc(-c4ccc5ccccc5c4)nc(-c4cccc5oc6cc(-c7ccccc7)ccc6c45)n3)cc2)cc1 ; c1ccc(-c2ccc3cc(-c4nc(-c5ccccc5)nc(-c5cccc6oc7cc(-c8ccccc8)ccc7c56)n4)ccc3c2)cc1]

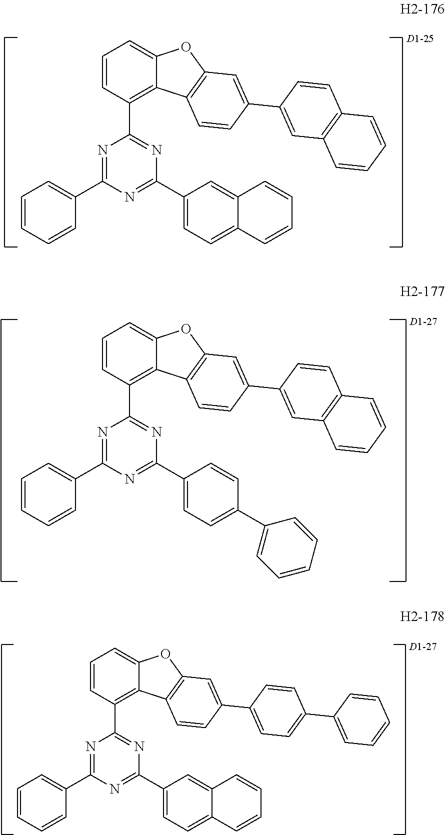

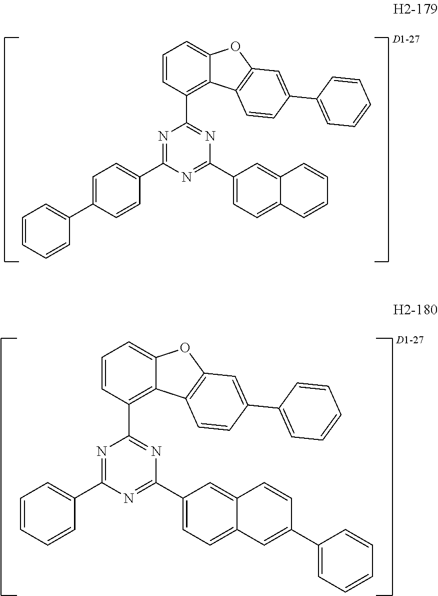

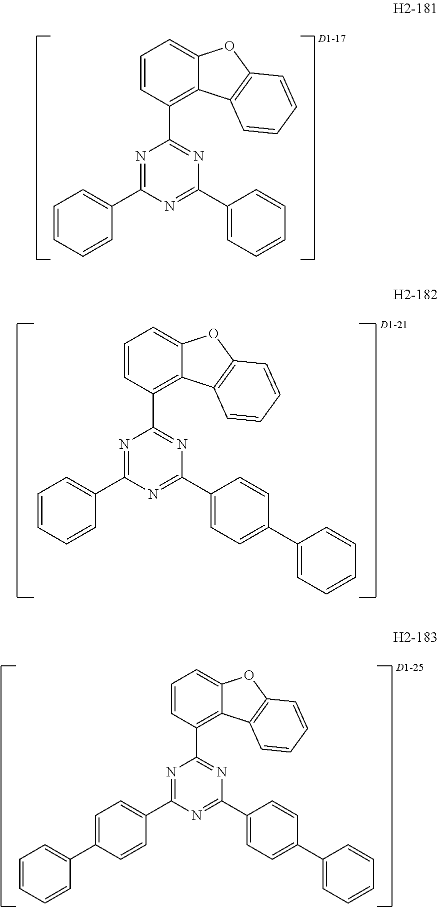

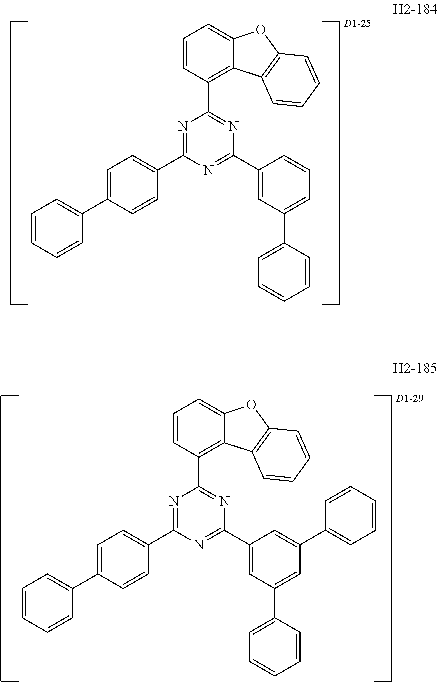

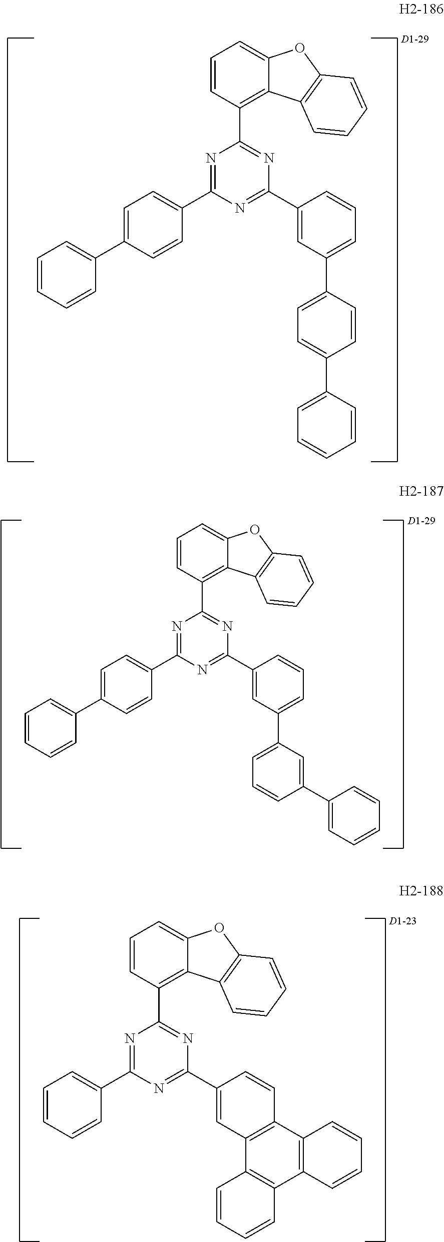

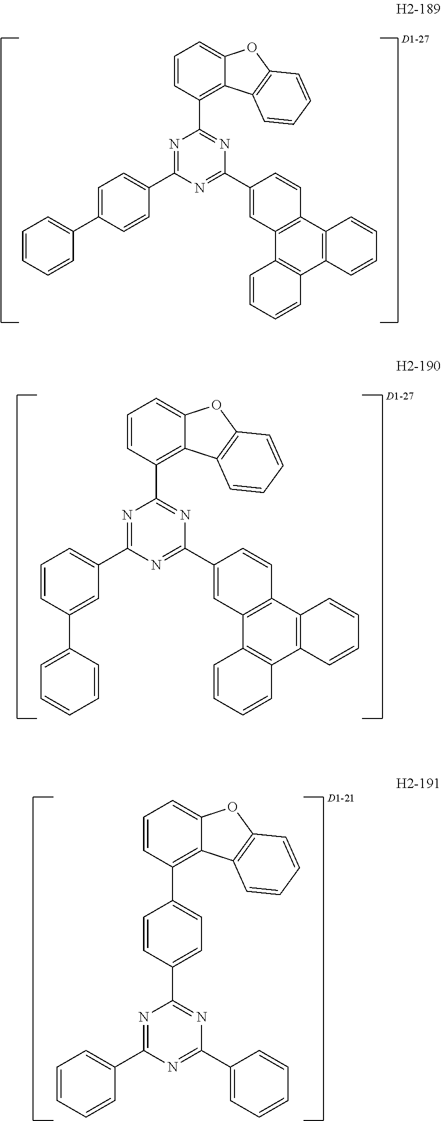

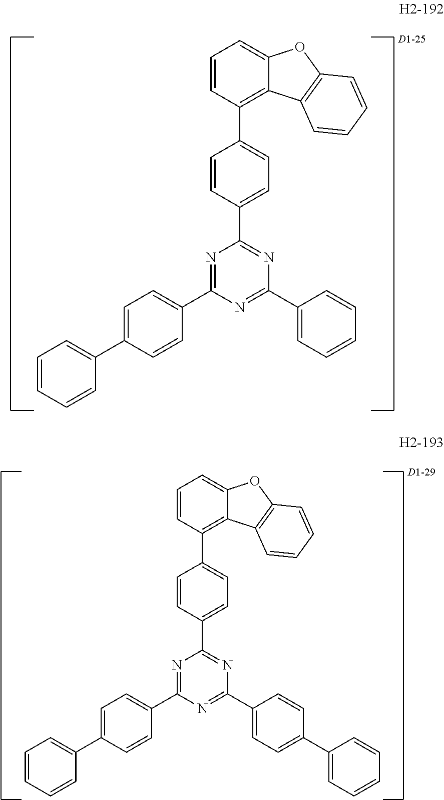

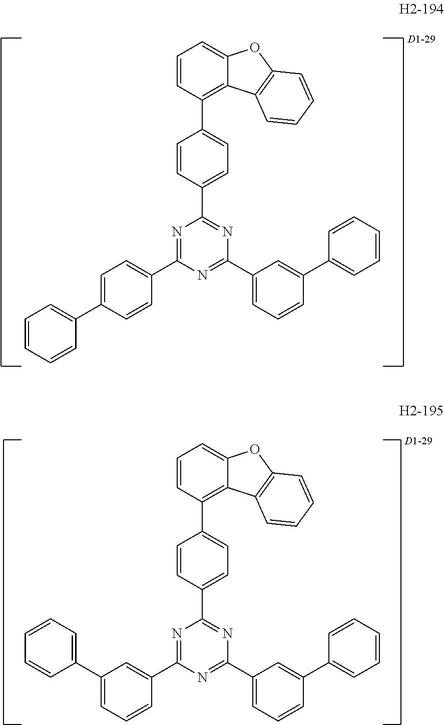

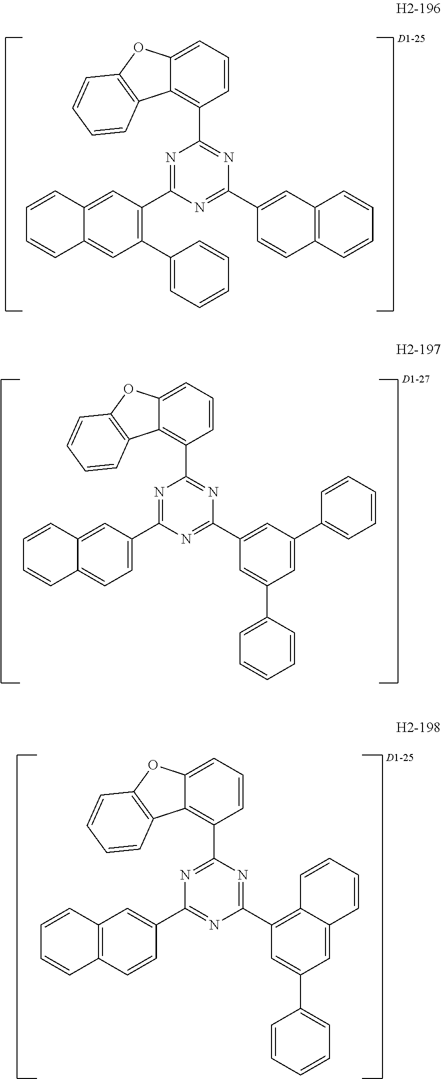

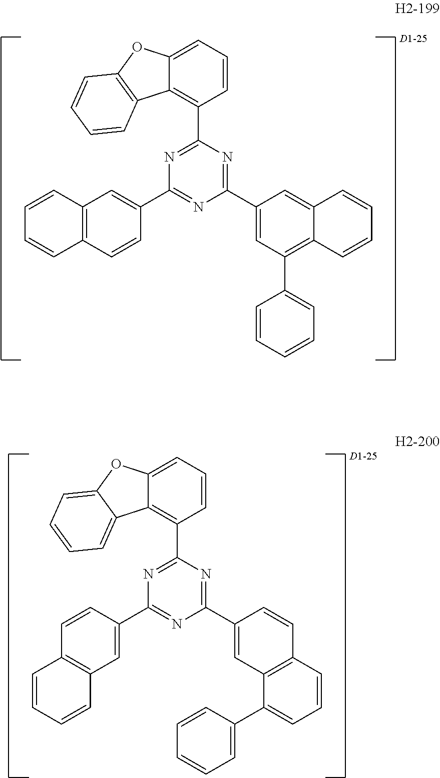

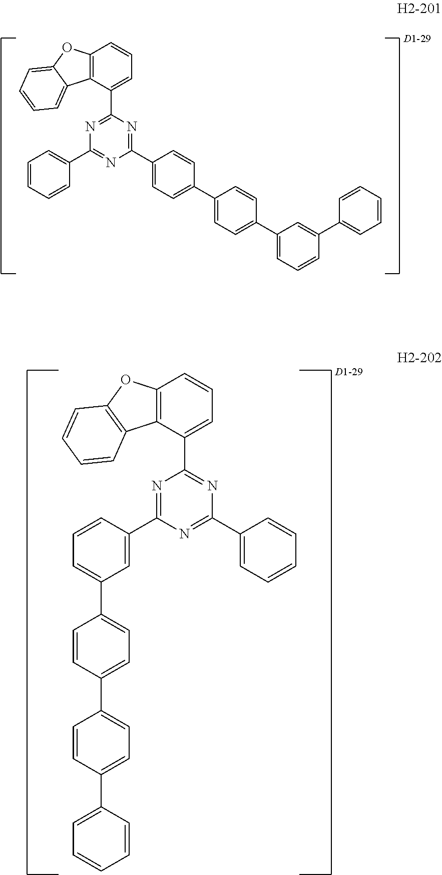

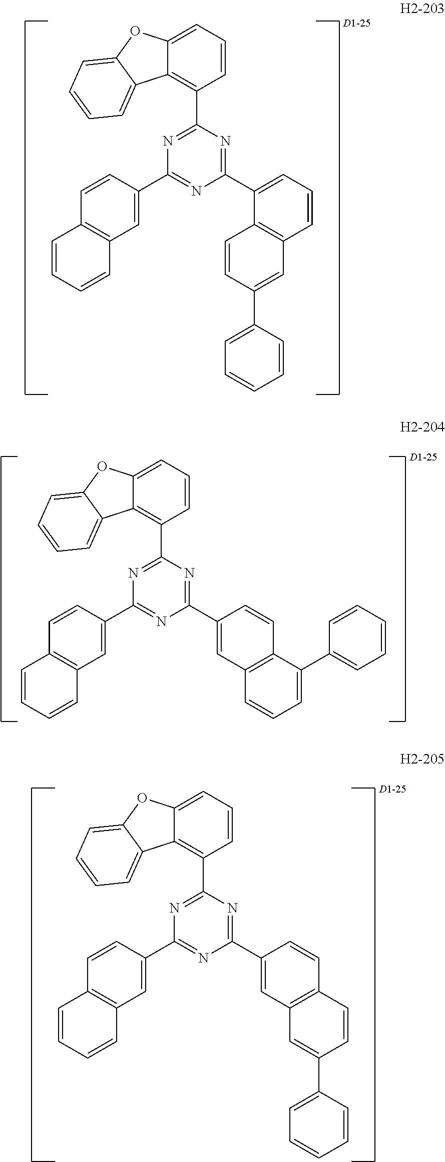

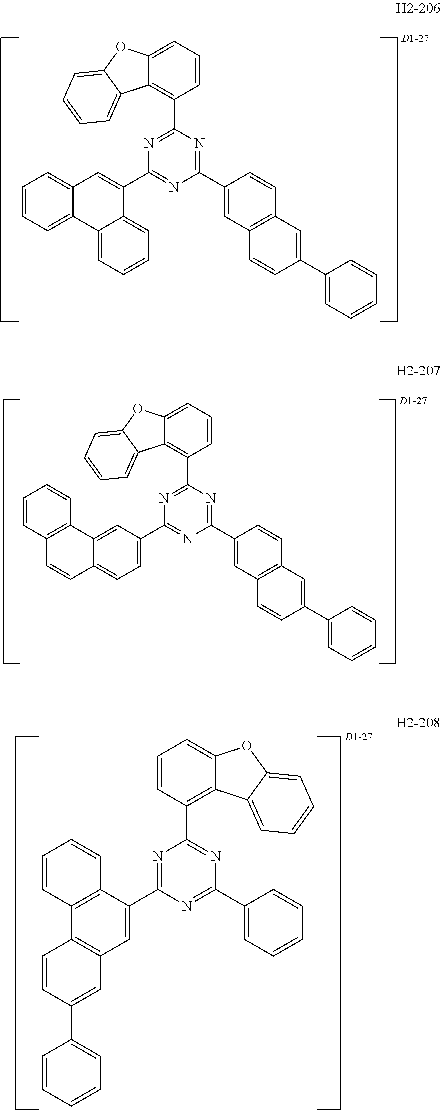

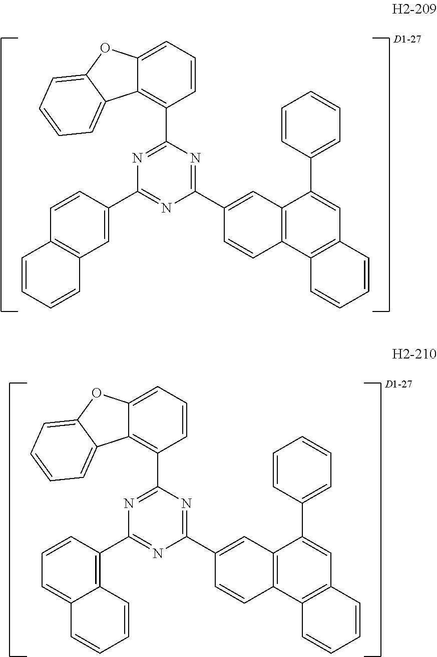

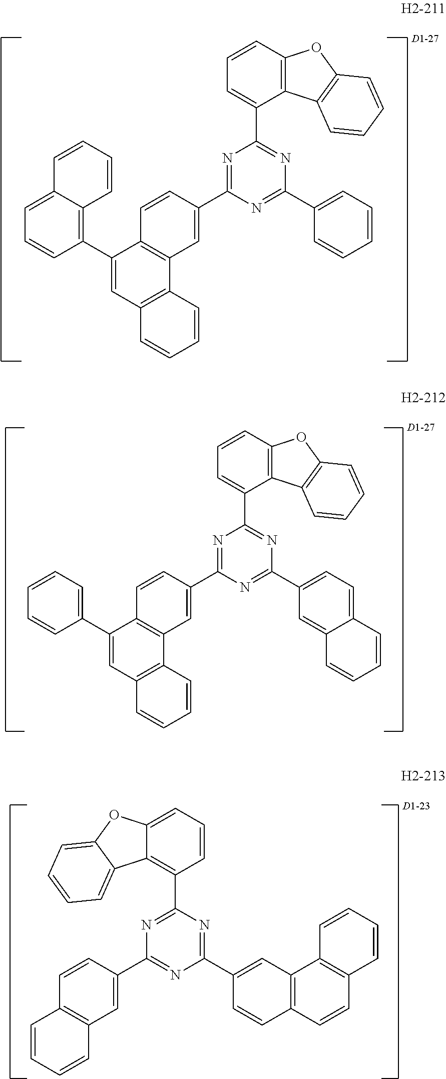

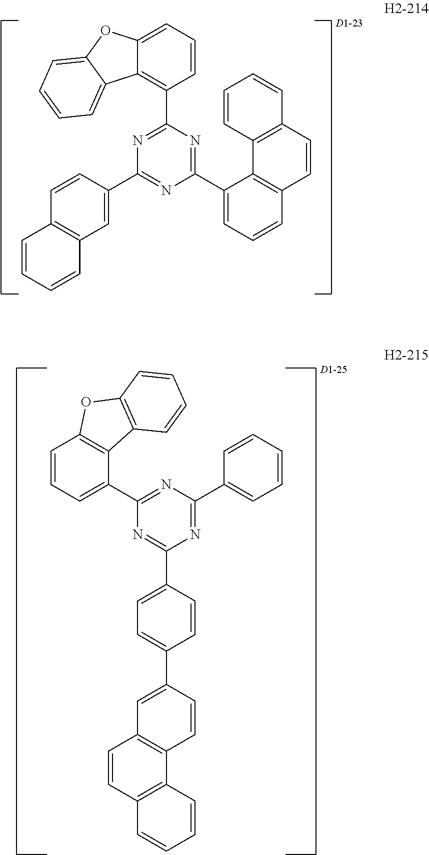

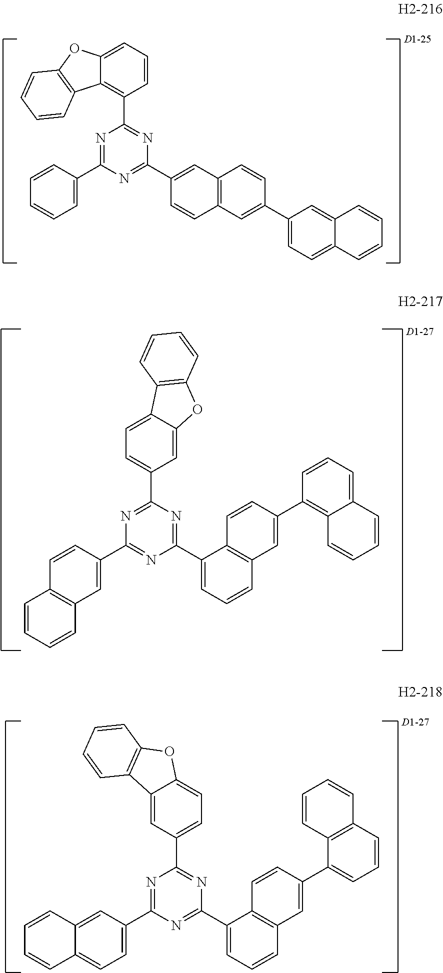

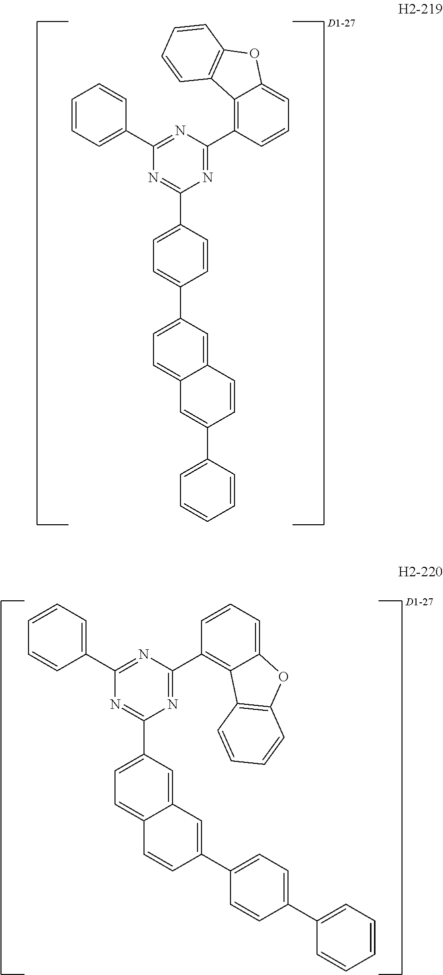

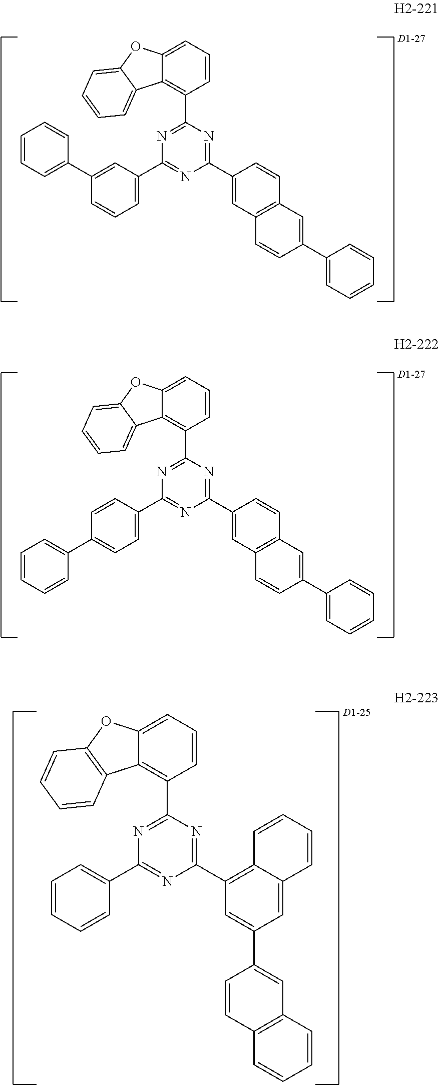

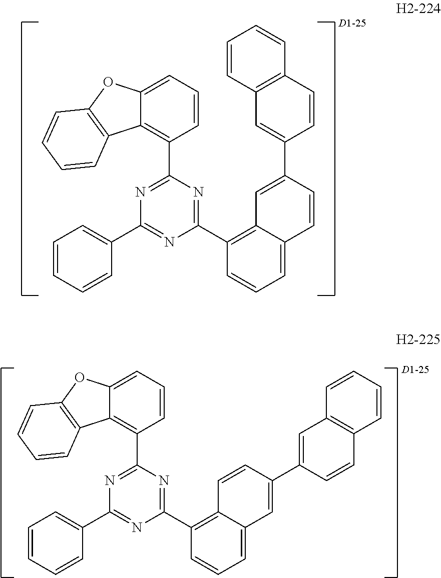

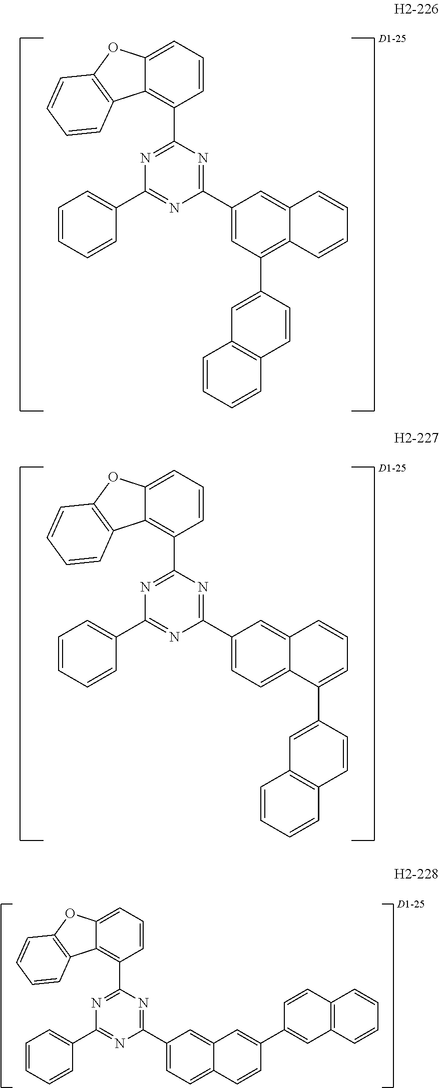

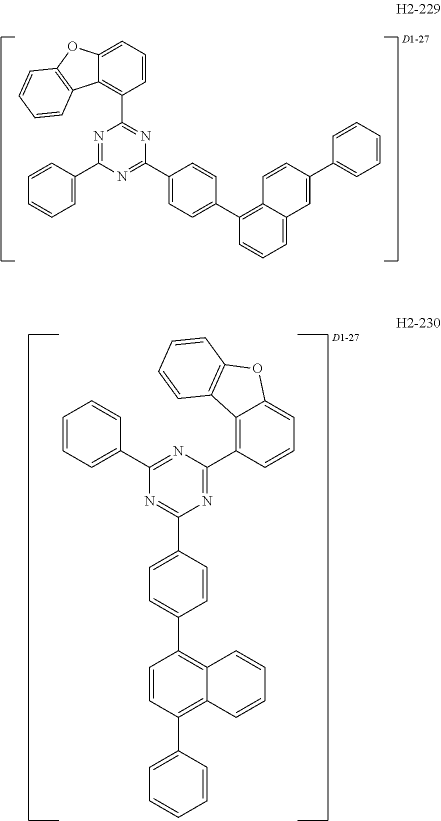

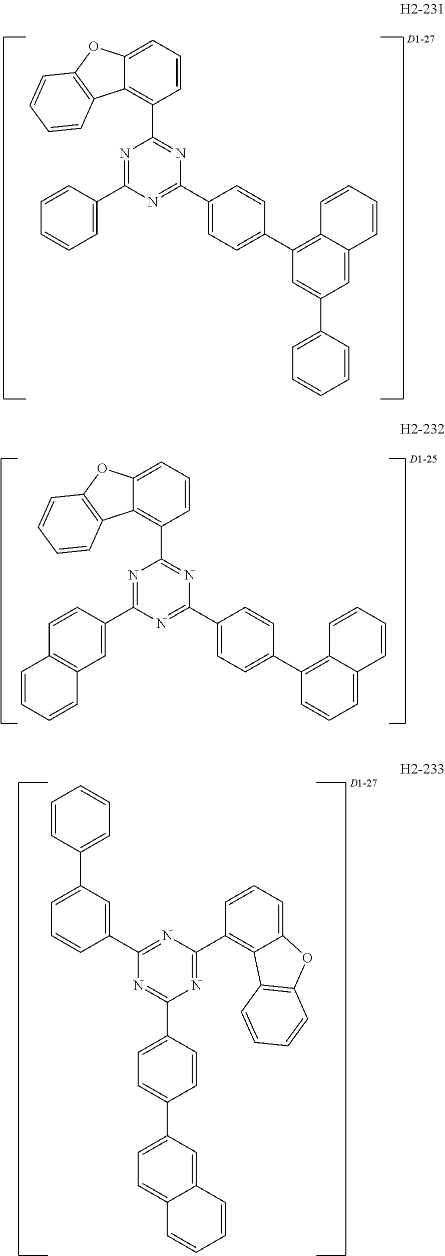

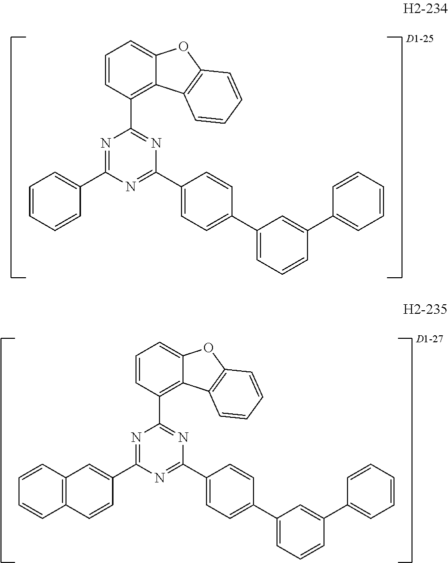

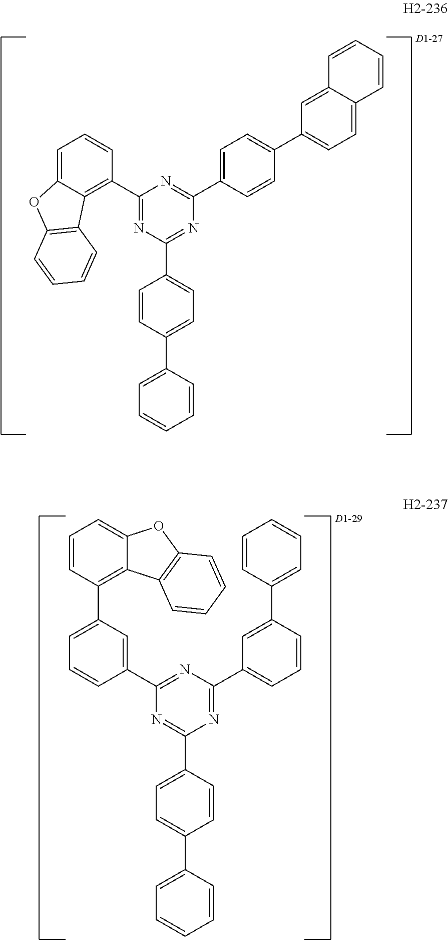

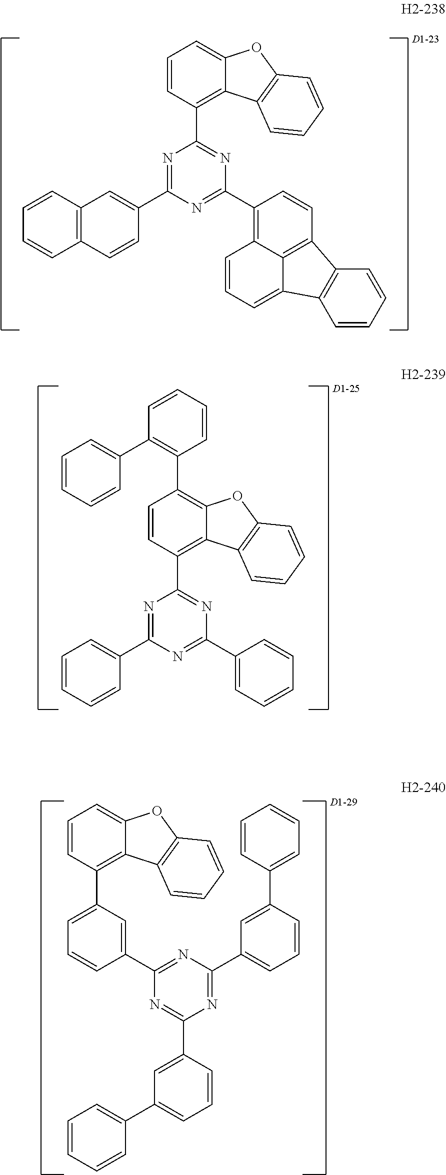

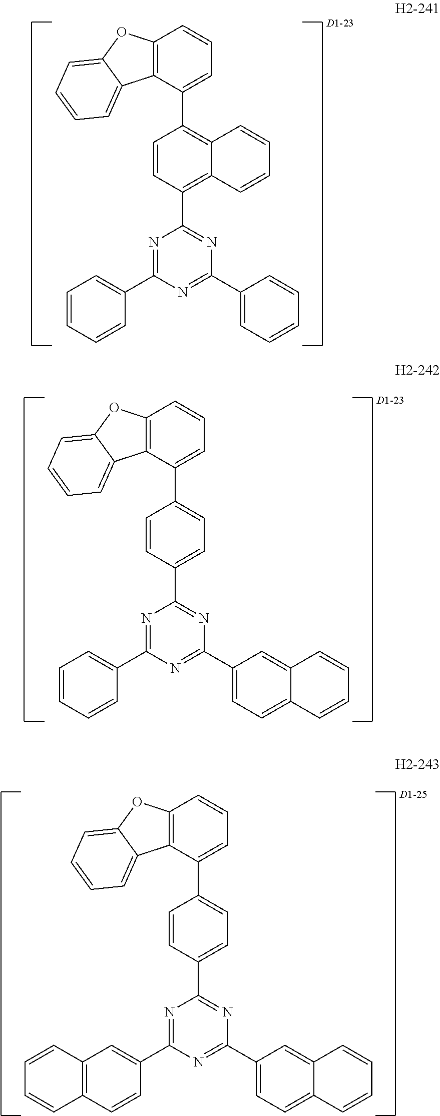

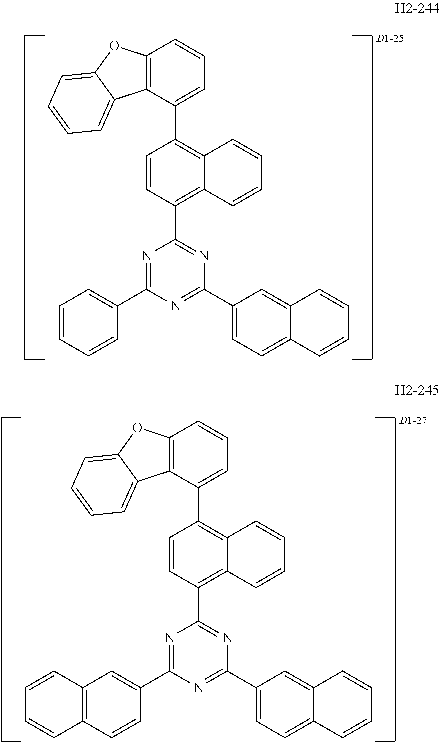

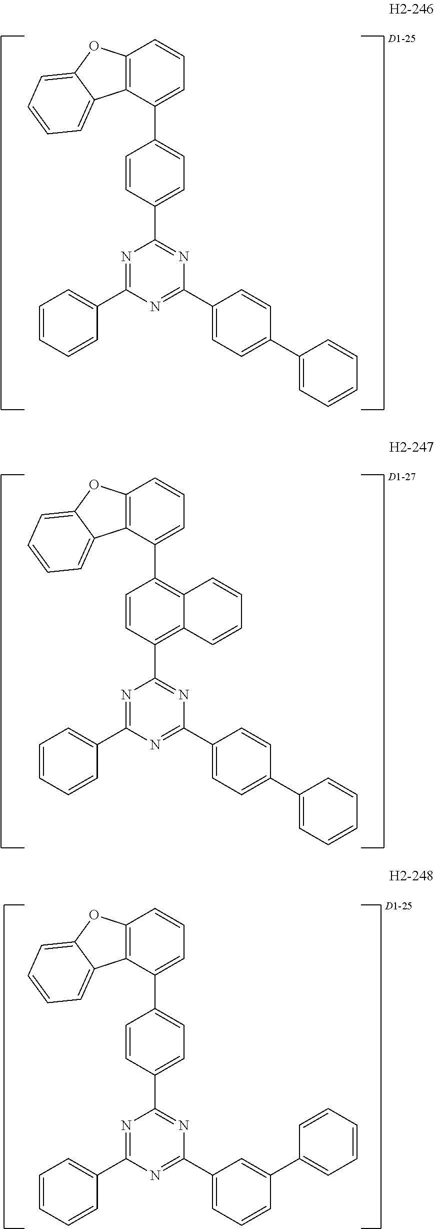

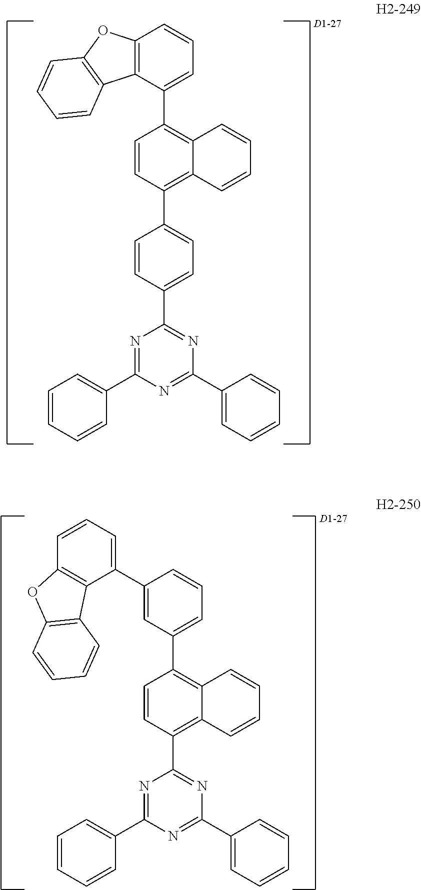

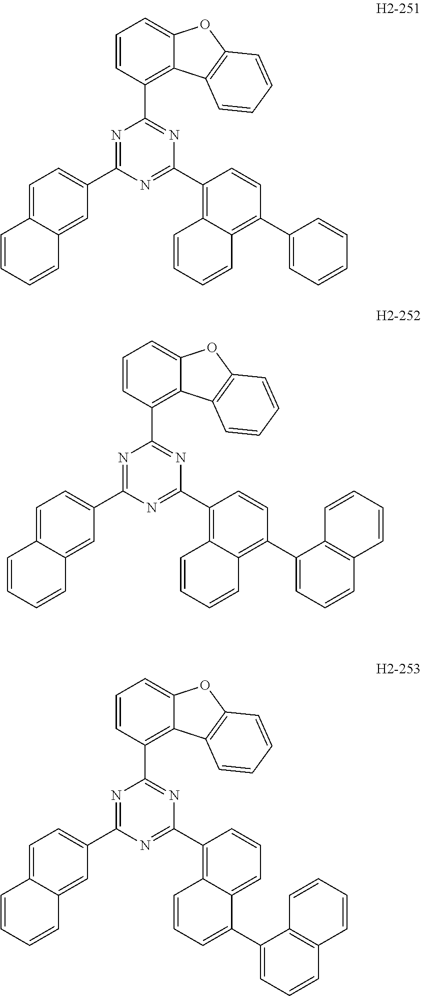 [SMILES: c1ccc(-c2ccc(-c3nc(-c4ccc5ccccc5c4)nc(-c4cccc5oc6ccccc6c45)n3)c3ccccc23)cc1 ; c1ccc2cc(-c3nc(-c4ccc(-c5cccc6ccccc56)c5ccccc45)nc(-c4cccc5oc6ccccc6c45)n3)ccc2c1 ; c1ccc2cc(-c3nc(-c4cccc5c(-c6cccc7ccccc67)cccc45)nc(-c4cccc5oc6ccccc6c45)n3)ccc2c1]

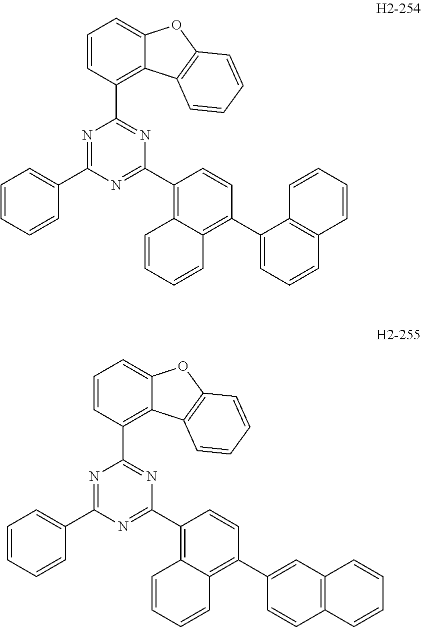 [SMILES: c1ccc(-c2nc(-c3ccc(-c4cccc5ccccc45)c4ccccc34)nc(-c3cccc4oc5ccccc5c34)n2)cc1 ; c1ccc(-c2nc(-c3ccc(-c4ccc5ccccc5c4)c4ccccc34)nc(-c3cccc4oc5ccccc5c34)n2)cc1]

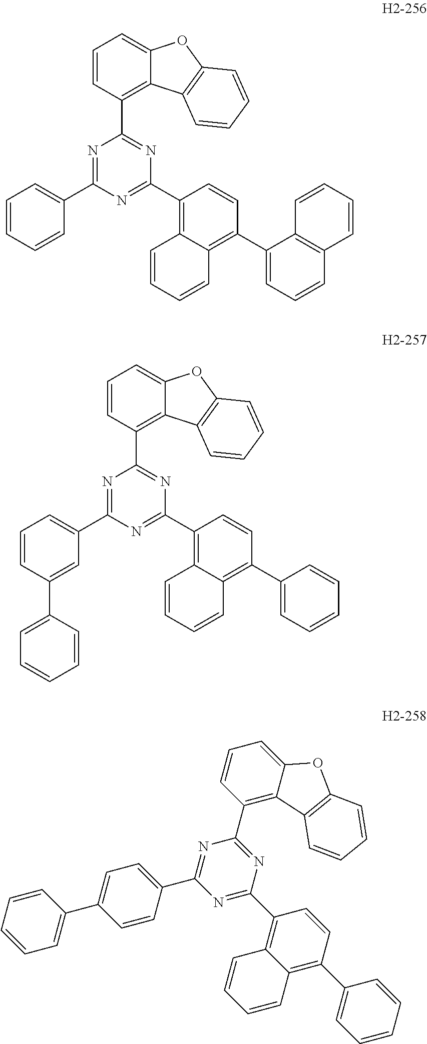 [SMILES: c1ccc(-c2nc(-c3ccc(-c4cccc5ccccc45)c4ccccc34)nc(-c3cccc4oc5ccccc5c34)n2)cc1 ; c1ccc(-c2cccc(-c3nc(-c4ccc(-c5ccccc5)c5ccccc45)nc(-c4cccc5oc6ccccc6c45)n3)c2)cc1 ; c1ccc(-c2ccc(-c3nc(-c4ccc(-c5ccccc5)c5ccccc45)nc(-c4cccc5oc6ccccc6c45)n3)cc2)cc1]

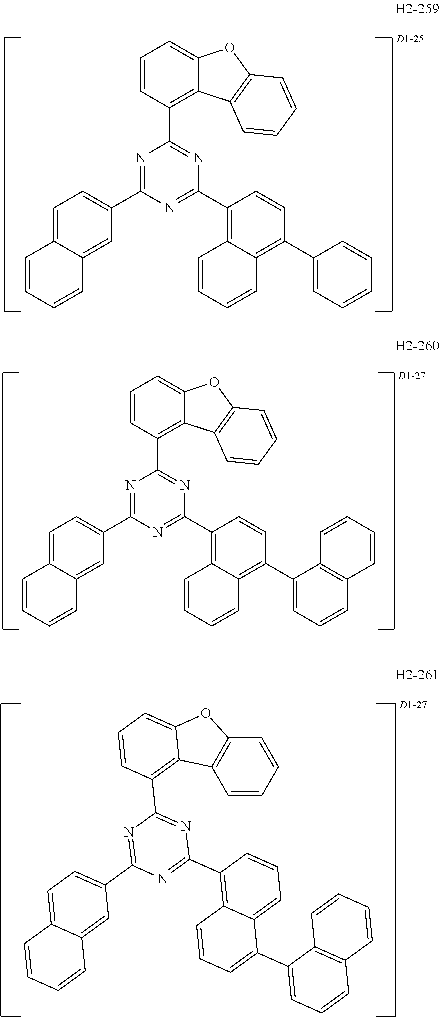

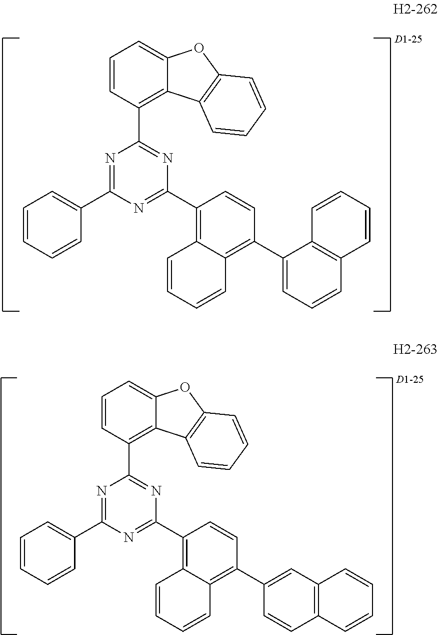

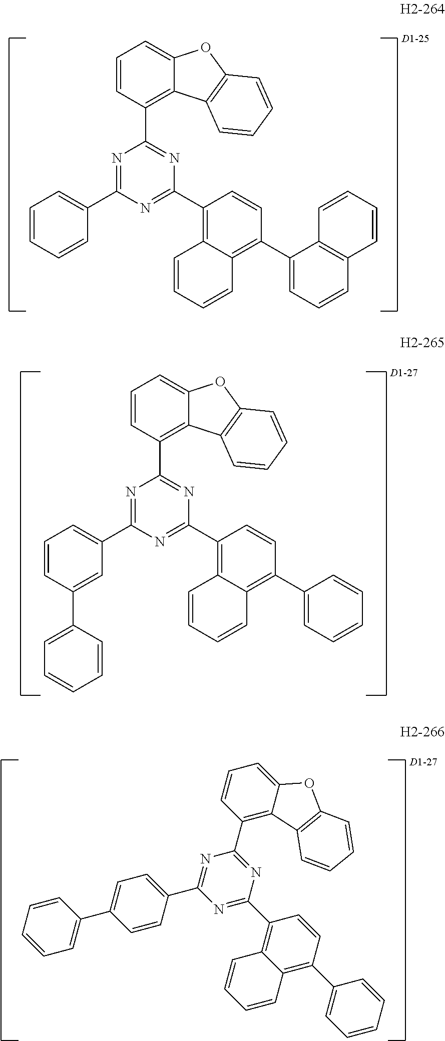

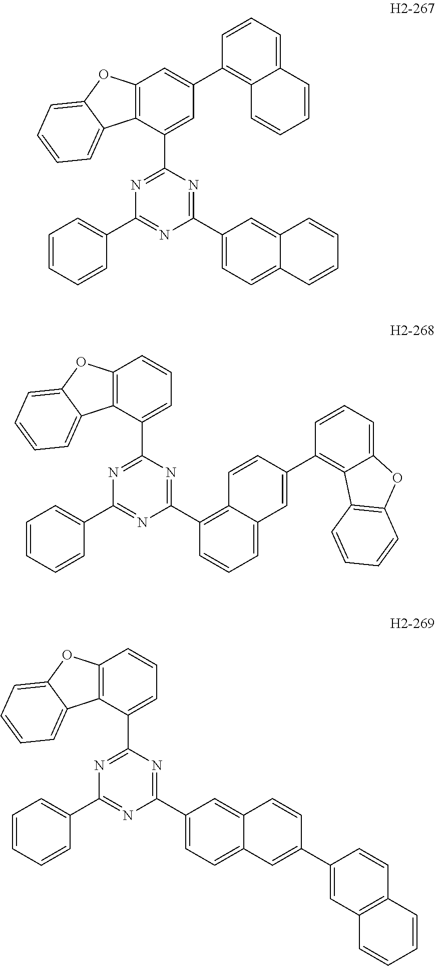 [SMILES: c1ccc(-c2nc(-c3ccc4ccccc4c3)nc(-c3cc(-c4cccc5ccccc45)cc4oc5ccccc5c34)n2)cc1 ; c1ccc(-c2nc(-c3cccc4cc(-c5cccc6oc7ccccc7c56)ccc34)nc(-c3cccc4oc5ccccc5c34)n2)cc1 ; c1ccc(-c2nc(-c3ccc4cc(-c5ccc6ccccc6c5)ccc4c3)nc(-c3cccc4oc5ccccc5c34)n2)cc1]

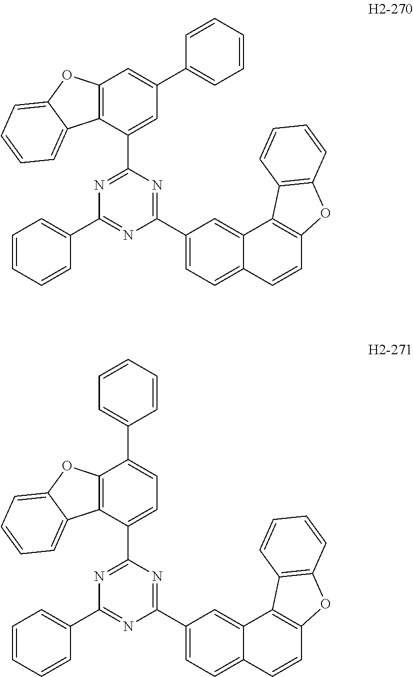 [SMILES: c1ccc(-c2cc(-c3nc(-c4ccccc4)nc(-c4ccc5ccc6oc7ccccc7c6c5c4)n3)c3c(c2)oc2ccccc23)cc1 ; c1ccc(-c2nc(-c3ccc4ccc5oc6ccccc6c5c4c3)nc(-c3ccc(-c4ccccc4)c4oc5ccccc5c34)n2)cc1]

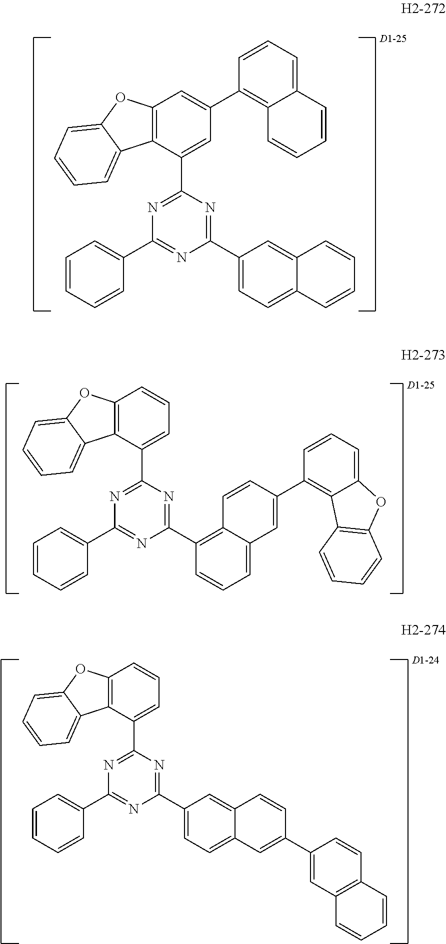

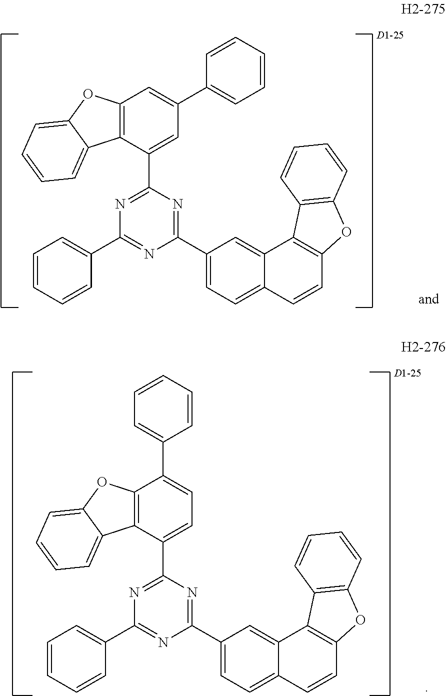

9. An organic electroluminescent device comprising an anode; a cathode; and at least one light-emitting layer between the anode and the cathode, wherein at least one layer of the light-emitting layers comprises the plurality of host materials according to claim 1.

10. The plurality of host materials according to claim 1, further comprising a third host material.

11. The plurality of host materials according to claim 10, wherein the third host material comprises the compound represented by at least one of the following formula 3: 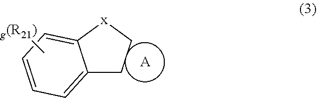
in formula 3,
X represents O, S, CR₃₁R₃₂, NR₃₃, or Se;
R₃₁ to R₃₃ each independently represent hydrogen, deuterium, a halogen, a substituted or unsubstituted (C1-C30)alkyl, a substituted or unsubstituted (C6-C30)aryl, or a substituted or unsubstituted (3- to 30-membered)heteroaryl; or may be linked to an adjacent substituent(s) to form a ring(s);
A represents a substituted or unsubstituted phenanthrene ring represented by the following formula 3-1; 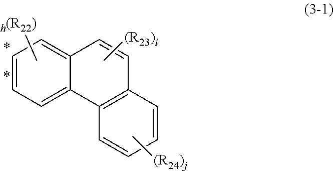
in formulas 3 and 3-1,
R₂₁ to R₂₄ each independently represent hydrogen, deuterium, a halogen, a substituted or unsubstituted (C1-C30)alkyl, a substituted or unsubstituted (C6-C30)aryl, 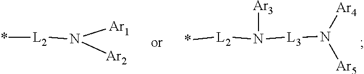
with a proviso that at least one of R₂₁ to R₂₄ represents 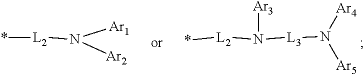
L₂ and L₃ each independently represent a single bond, a substituted or unsubstituted (C6-C30)arylene, a substituted or unsubstituted (C3-C30)cycloalkylene, or a substituted or unsubstituted (3- to 30-membered)heteroarylene;
Ar₁ to Ar₅ each independently represent a substituted or unsubstituted (C6-C30)aryl, or a substituted or unsubstituted (3- to 30-membered)heteroaryl;
g and j represent an integer of 1 to 4, h and i represent an integer of 1 or 2, where if g to h represent an integer of 2 or more, each of R₂₁ to each of R₂₄ may be the same as or different from each other;
* in formula 3-1 represents a site linked to formula 3; and
* in R₂₁ to R₂₄ represents a site linked to formula 3 or 3-1.

12. The plurality of host materials according to claim 11, wherein formula 3 is represented by the following formula 3-2 or 3-3: 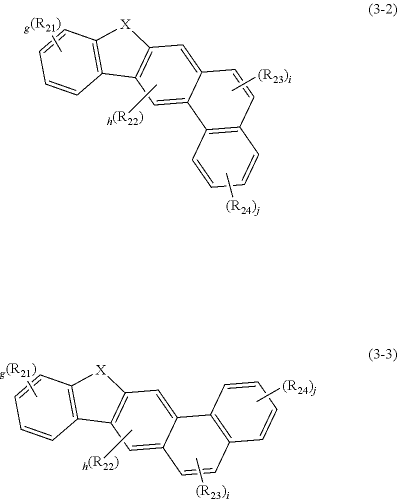
in formulas 3-2 and 3-3,
X, R₂₁ to R₂₄, and g to j are as defined in claim 11.

13. The plurality of host materials according to claim 10, wherein the compound represented by formula 3 is selected from the following compounds:

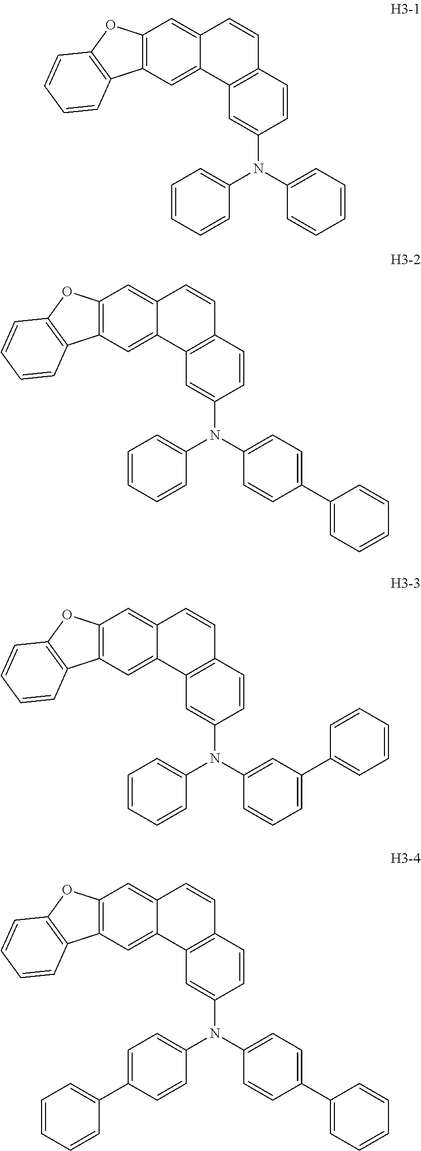 [SMILES: c1ccc(N(c2ccccc2)c2ccc3ccc4cc5oc6ccccc6c5cc4c3c2)cc1 ; c1ccc(-c2ccc(N(c3ccccc3)c3ccc4ccc5cc6oc7ccccc7c6cc5c4c3)cc2)cc1 ; c1ccc(-c2cccc(N(c3ccccc3)c3ccc4ccc5cc6oc7ccccc7c6cc5c4c3)c2)cc1 ; c1ccc(-c2ccc(N(c3ccc(-c4ccccc4)cc3)c3ccc4ccc5cc6oc7ccccc7c6cc5c4c3)cc2)cc1]

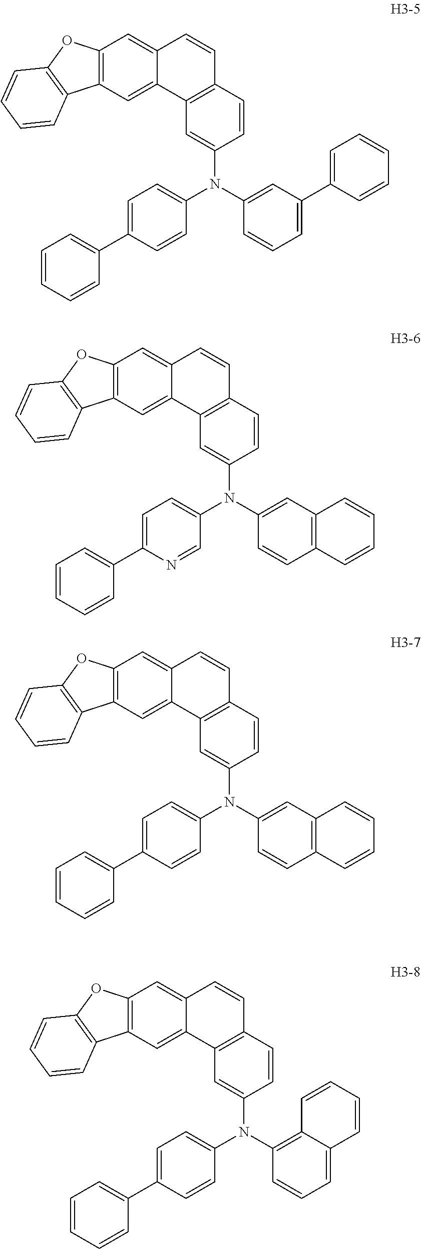

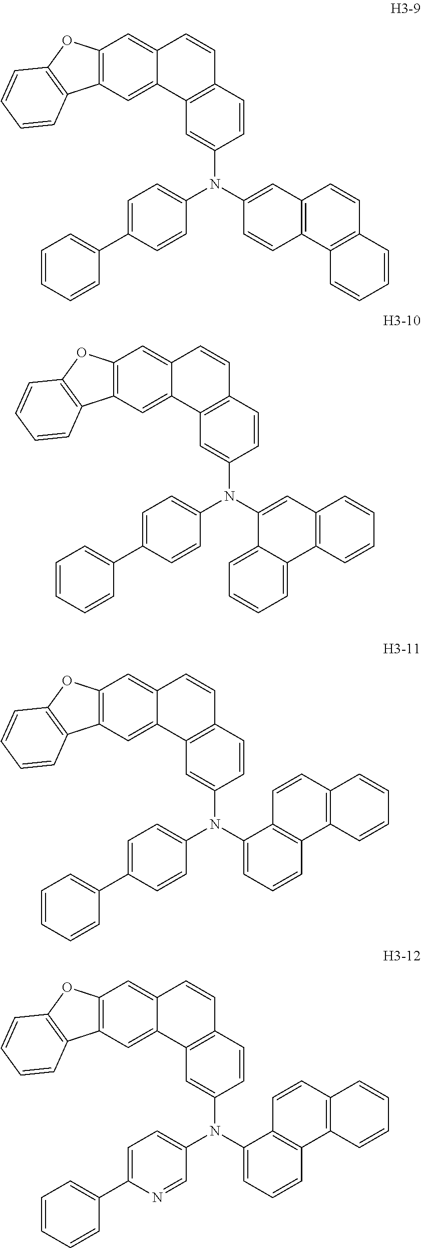

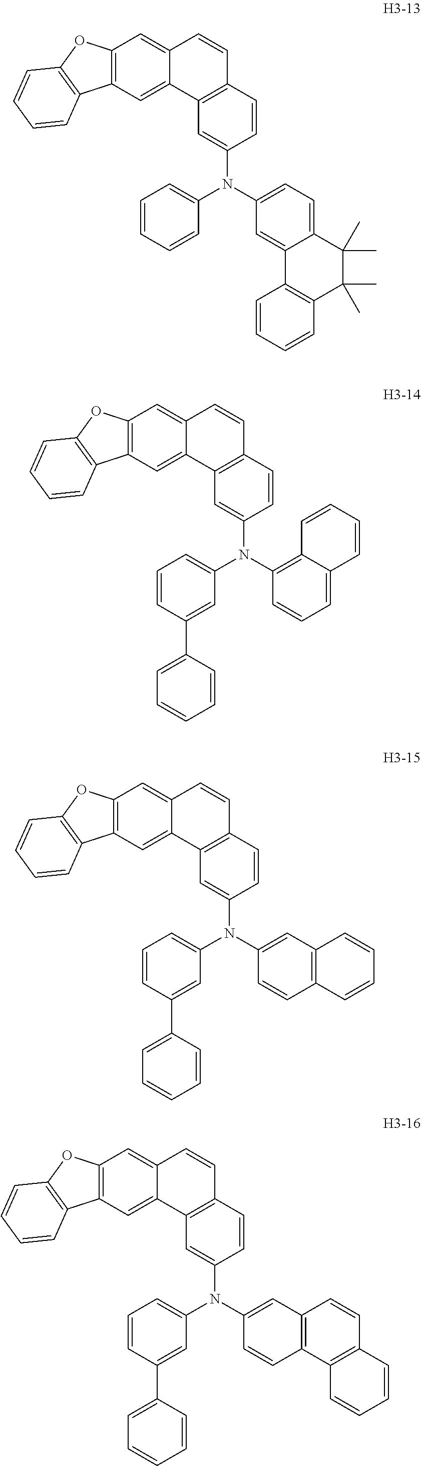 [SMILES: CC1(C)c2ccccc2-c2cc(N(c3ccccc3)c3ccc4ccc5cc6oc7ccccc7c6cc5c4c3)ccc2C1(C)C ; c1ccc(-c2cccc(N(c3ccc4ccc5cc6oc7ccccc7c6cc5c4c3)c3cccc4ccccc34)c2)cc1 ; c1ccc(-c2cccc(N(c3ccc4ccccc4c3)c3ccc4ccc5cc6oc7ccccc7c6cc5c4c3)c2)cc1 ; c1ccc(-c2cccc(N(c3ccc4c(ccc5ccccc54)c3)c3ccc4ccc5cc6oc7ccccc7c6cc5c4c3)c2)cc1]

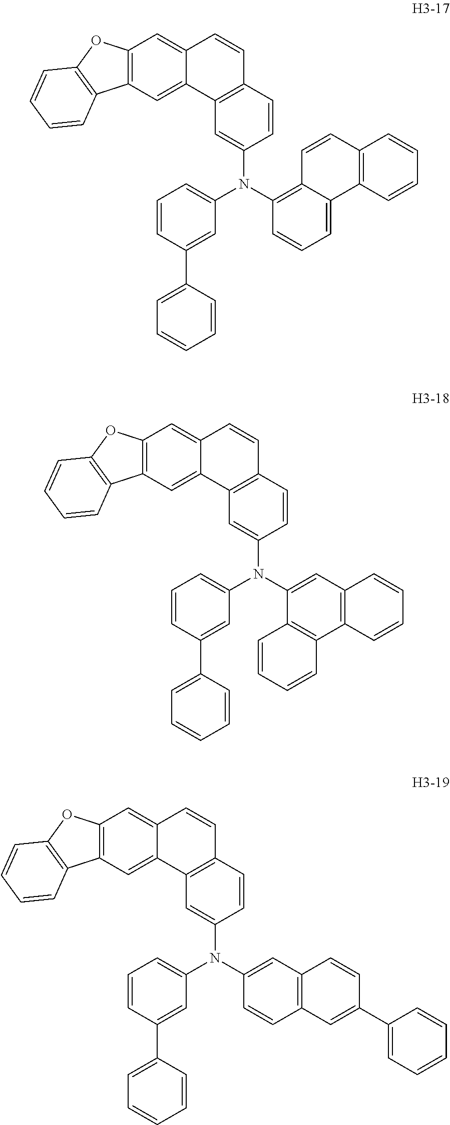 [SMILES: c1ccc(-c2cccc(N(c3ccc4ccc5cc6oc7ccccc7c6cc5c4c3)c3cccc4c3ccc3ccccc34)c2)cc1 ; c1ccc(-c2cccc(N(c3ccc4ccc5cc6oc7ccccc7c6cc5c4c3)c3cc4ccccc4c4ccccc34)c2)cc1 ; c1ccc(-c2cccc(N(c3ccc4cc(-c5ccccc5)ccc4c3)c3ccc4ccc5cc6oc7ccccc7c6cc5c4c3)c2)cc1]

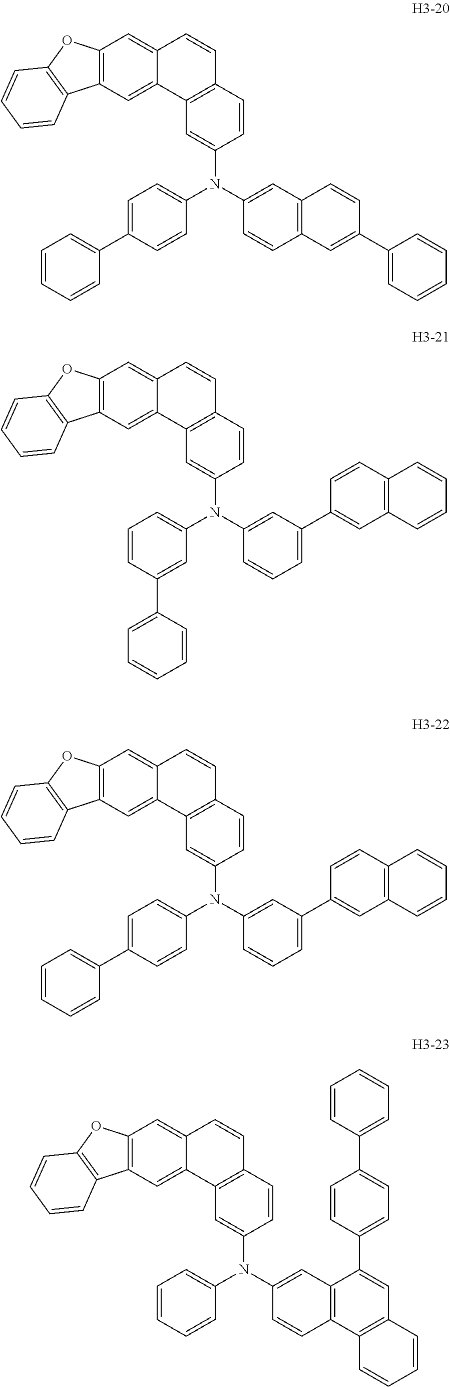 [SMILES: c1ccc(-c2ccc(N(c3ccc4cc(-c5ccccc5)ccc4c3)c3ccc4ccc5cc6oc7ccccc7c6cc5c4c3)cc2)cc1 ; c1ccc(-c2cccc(N(c3cccc(-c4ccc5ccccc5c4)c3)c3ccc4ccc5cc6oc7ccccc7c6cc5c4c3)c2)cc1 ; c1ccc(-c2ccc(N(c3cccc(-c4ccc5ccccc5c4)c3)c3ccc4ccc5cc6oc7ccccc7c6cc5c4c3)cc2)cc1 ; c1ccc(-c2ccc(-c3cc4ccccc4c4ccc(N(c5ccccc5)c5ccc6ccc7cc8oc9ccccc9c8cc7c6c5)cc34)cc2)cc1]

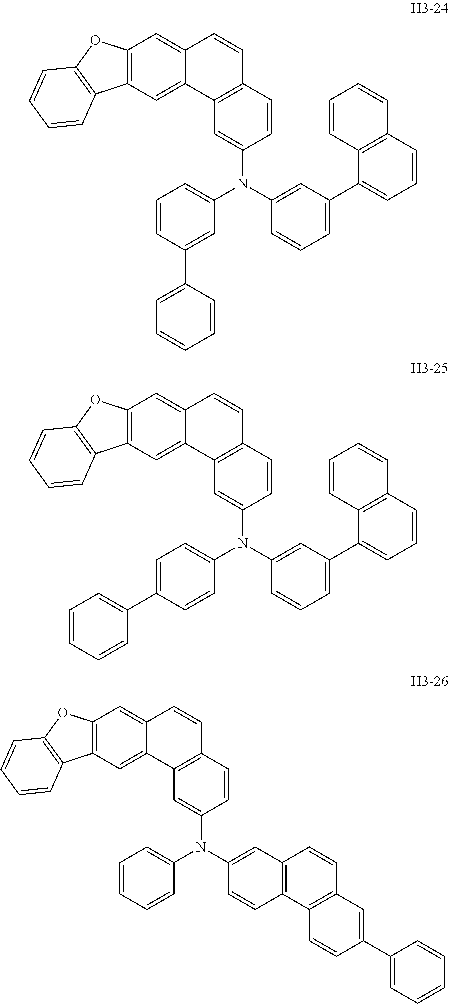 [SMILES: c1ccc(-c2cccc(N(c3cccc(-c4cccc5ccccc45)c3)c3ccc4ccc5cc6oc7ccccc7c6cc5c4c3)c2)cc1 ; c1ccc(-c2ccc(N(c3cccc(-c4cccc5ccccc45)c3)c3ccc4ccc5cc6oc7ccccc7c6cc5c4c3)cc2)cc1 ; c1ccc(-c2ccc3c(ccc4cc(N(c5ccccc5)c5ccc6ccc7cc8oc9ccccc9c8cc7c6c5)ccc43)c2)cc1]

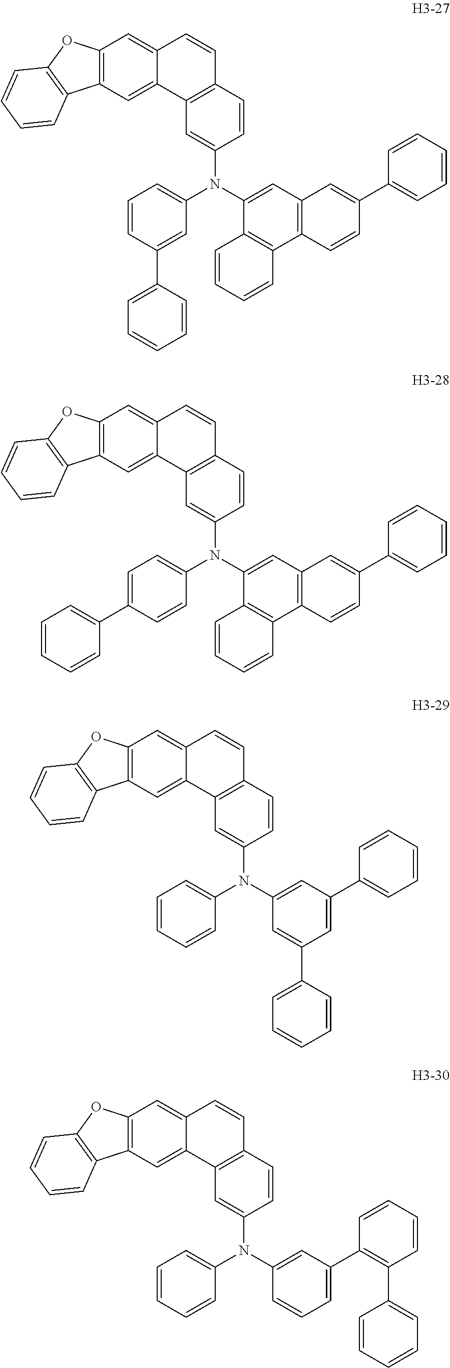 [SMILES: c1ccc(-c2cccc(N(c3ccc4ccc5cc6oc7ccccc7c6cc5c4c3)c3cc4cc(-c5ccccc5)ccc4c4ccccc34)c2)cc1 ; c1ccc(-c2ccc(N(c3ccc4ccc5cc6oc7ccccc7c6cc5c4c3)c3cc4cc(-c5ccccc5)ccc4c4ccccc34)cc2)cc1 ; c1ccc(-c2cc(-c3ccccc3)cc(N(c3ccccc3)c3ccc4ccc5cc6oc7ccccc7c6cc5c4c3)c2)cc1 ; c1ccc(-c2ccccc2-c2cccc(N(c3ccccc3)c3ccc4ccc5cc6oc7ccccc7c6cc5c4c3)c2)cc1]

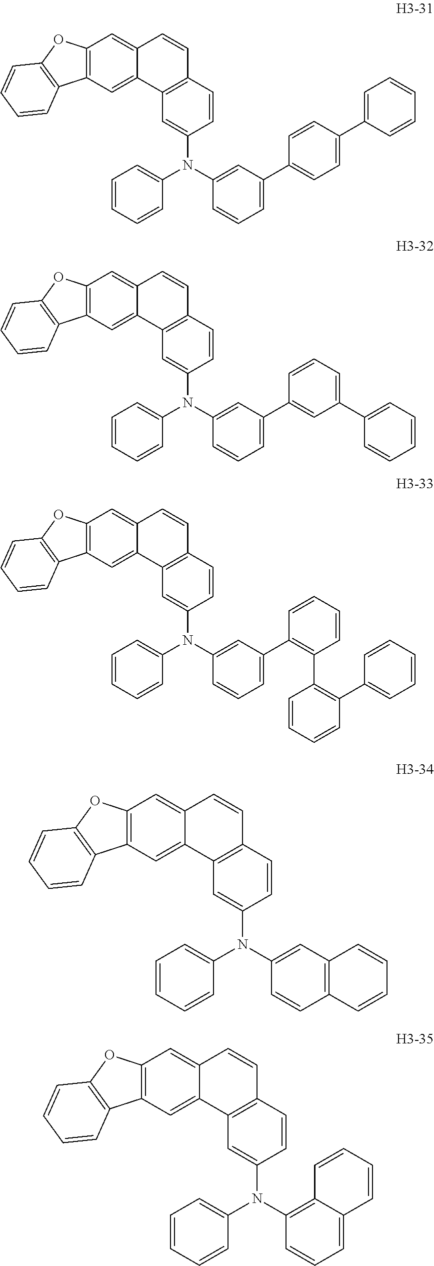

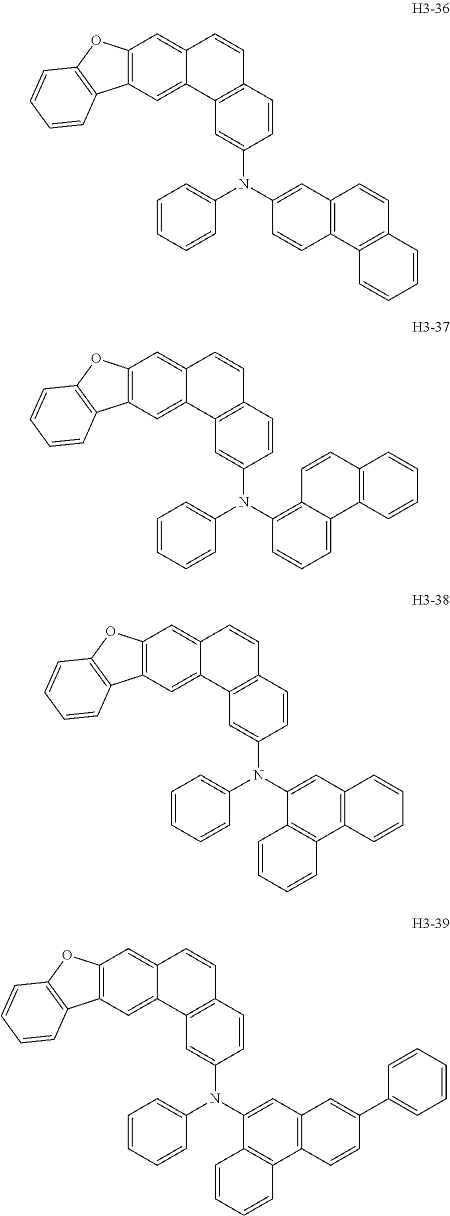 [SMILES: c1ccc(N(c2ccc3c(ccc4ccccc43)c2)c2ccc3ccc4cc5oc6ccccc6c5cc4c3c2)cc1 ; c1ccc(N(c2ccc3ccc4cc5oc6ccccc6c5cc4c3c2)c2cccc3c2ccc2ccccc23)cc1 ; c1ccc(N(c2ccc3ccc4cc5oc6ccccc6c5cc4c3c2)c2cc3ccccc3c3ccccc23)cc1 ; c1ccc(-c2ccc3c(c2)cc(N(c2ccccc2)c2ccc4ccc5cc6oc7ccccc7c6cc5c4c2)c2ccccc23)cc1]

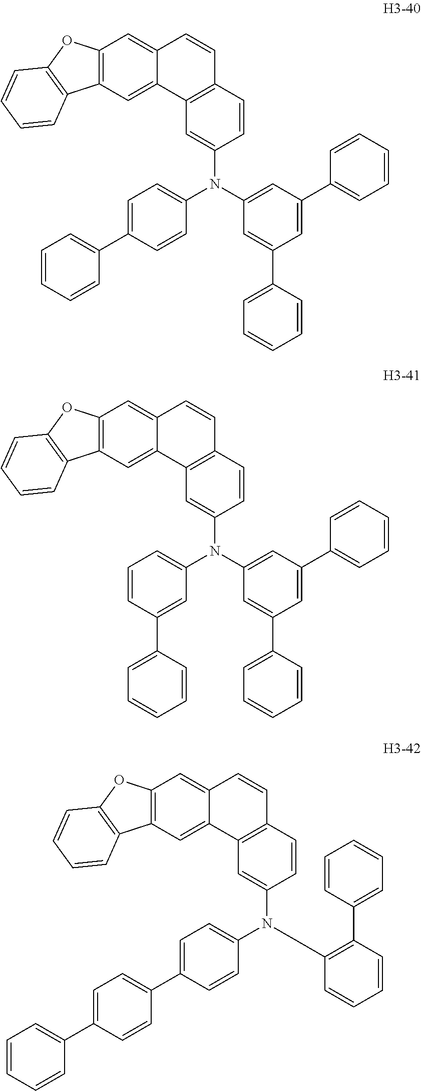 [SMILES: c1ccc(-c2ccc(N(c3cc(-c4ccccc4)cc(-c4ccccc4)c3)c3ccc4ccc5cc6oc7ccccc7c6cc5c4c3)cc2)cc1 ; c1ccc(-c2cccc(N(c3cc(-c4ccccc4)cc(-c4ccccc4)c3)c3ccc4ccc5cc6oc7ccccc7c6cc5c4c3)c2)cc1 ; c1ccc(-c2ccc(-c3ccc(N(Cc4ccccc4-c4ccccc4)c4ccc5ccc6cc7oc8ccccc8c7cc6c5c4)cc3)cc2)cc1]

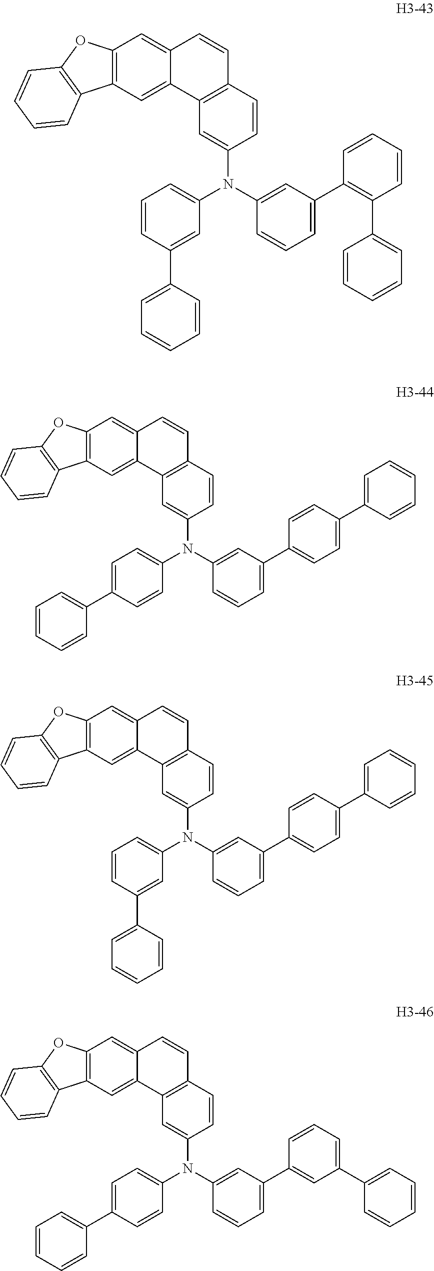 [SMILES: c1ccc(-c2cccc(N(c3cccc(-c4ccccc4-c4ccccc4)c3)c3ccc4ccc5cc6oc7ccccc7c6cc5c4c3)c2)cc1 ; c1ccc(-c2ccc(-c3cccc(N(c4ccc(-c5ccccc5)cc4)c4ccc5ccc6cc7oc8ccccc8c7cc6c5c4)c3)cc2)cc1 ; c1ccc(-c2ccc(-c3cccc(N(c4cccc(-c5ccccc5)c4)c4ccc5ccc6cc7oc8ccccc8c7cc6c5c4)c3)cc2)cc1 ; c1ccc(-c2ccc(N(c3cccc(-c4cccc(-c5ccccc5)c4)c3)c3ccc4ccc5cc6oc7ccccc7c6cc5c4c3)cc2)cc1]

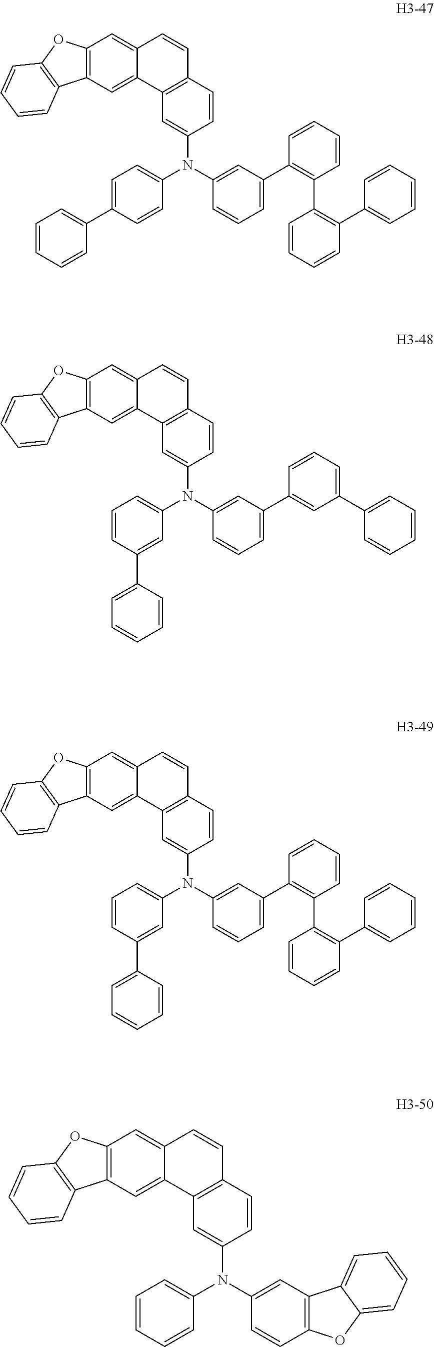 [SMILES: c1ccc(-c2ccc(N(c3cccc(-c4ccccc4-c4ccccc4-c4ccccc4)c3)c3ccc4ccc5cc6oc7ccccc7c6cc5c4c3)cc2)cc1 ; c1ccc(-c2cccc(-c3cccc(N(c4cccc(-c5ccccc5)c4)c4ccc5ccc6cc7oc8ccccc8c7cc6c5c4)c3)c2)cc1 ; c1ccc(-c2cccc(N(c3cccc(-c4ccccc4-c4ccccc4-c4ccccc4)c3)c3ccc4ccc5cc6oc7ccccc7c6cc5c4c3)c2)cc1 ; c1ccc(N(c2ccc3ccc4cc5oc6ccccc6c5cc4c3c2)c2ccc3oc4ccccc4c3c2)cc1]

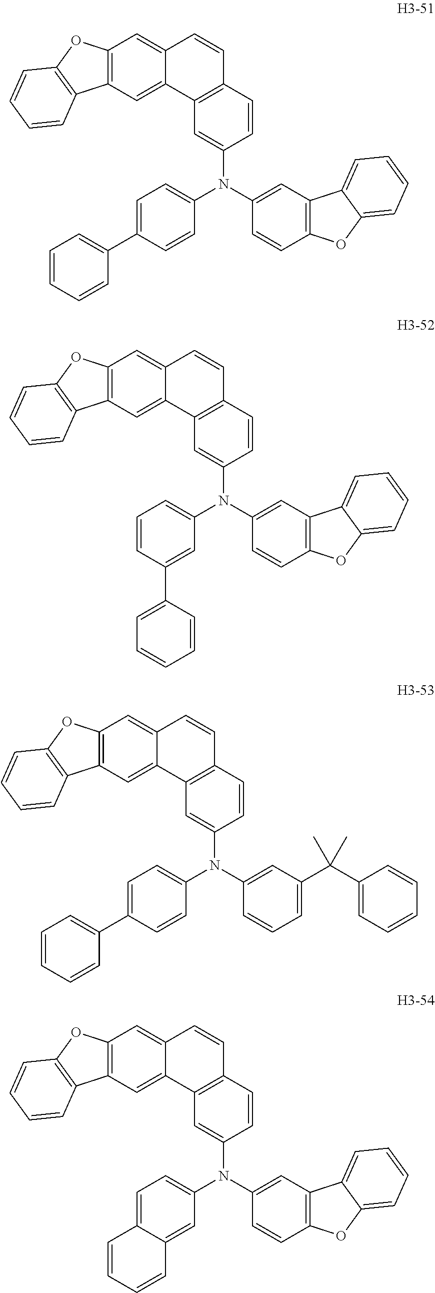 [SMILES: c1ccc(-c2ccc(N(c3ccc4ccc5cc6oc7ccccc7c6cc5c4c3)c3ccc4oc5ccccc5c4c3)cc2)cc1 ; c1ccc(-c2cccc(N(c3ccc4ccc5cc6oc7ccccc7c6cc5c4c3)c3ccc4oc5ccccc5c4c3)c2)cc1 ; CC(C)(c1ccccc1)c1cccc(N(c2ccc(-c3ccccc3)cc2)c2ccc3ccc4cc5oc6ccccc6c5cc4c3c2)c1 ; c1ccc2cc(N(c3ccc4ccc5cc6oc7ccccc7c6cc5c4c3)c3ccc4oc5ccccc5c4c3)ccc2c1]

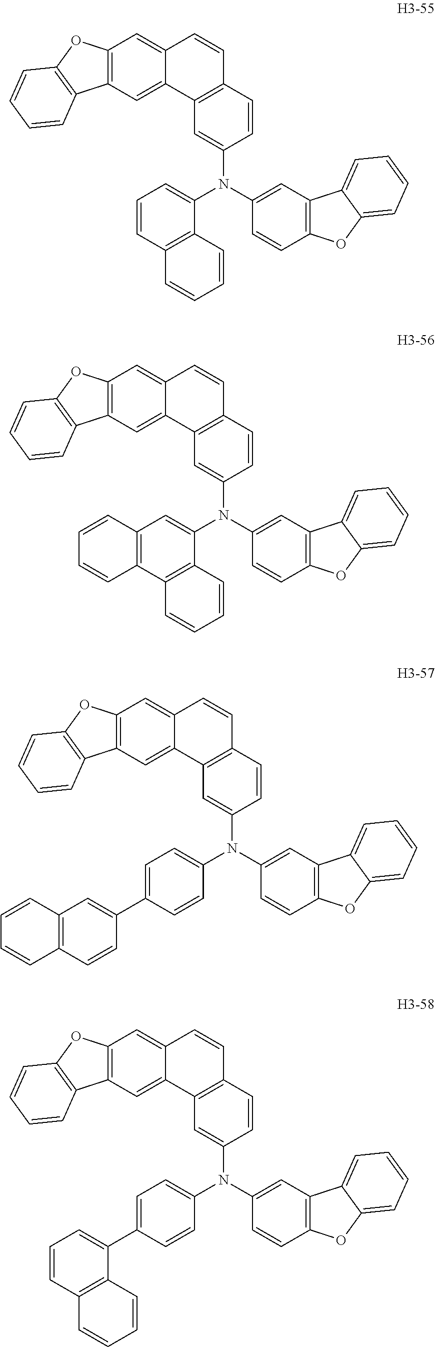 [SMILES: c1ccc2c(N(c3ccc4ccc5cc6oc7ccccc7c6cc5c4c3)c3ccc4oc5ccccc5c4c3)cccc2c1 ; c1ccc2c(c1)cc(N(c1ccc3ccc4cc5oc6ccccc6c5cc4c3c1)c1ccc3oc4ccccc4c3c1)c1ccccc12 ; c1ccc2cc(-c3ccc(N(c4ccc5ccc6cc7oc8ccccc8c7cc6c5c4)c4ccc5oc6ccccc6c5c4)cc3)ccc2c1 ; c1ccc2c(-c3ccc(N(c4ccc5ccc6cc7oc8ccccc8c7cc6c5c4)c4ccc5oc6ccccc6c5c4)cc3)cccc2c1]

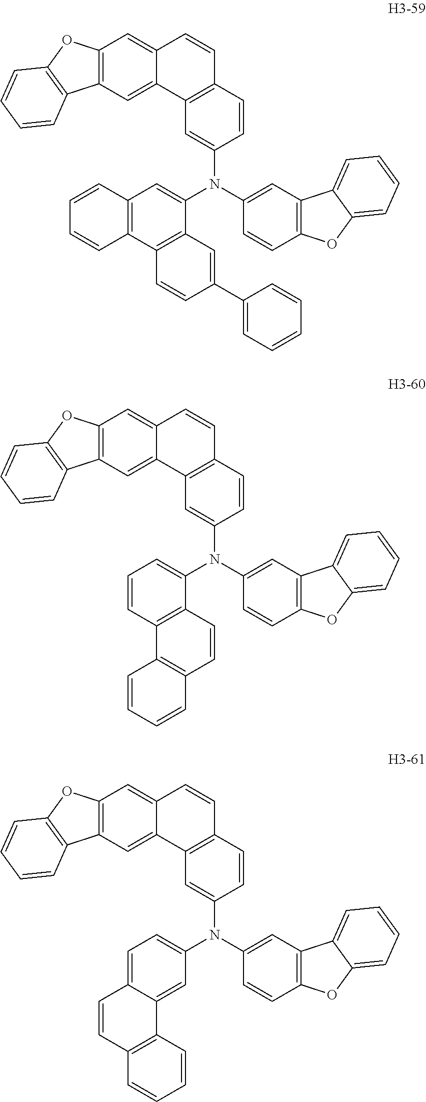 [SMILES: c1ccc(-c2ccc3c(c2)c(N(c2ccc4ccc5cc6oc7ccccc7c6cc5c4c2)c2ccc4oc5ccccc5c4c2)cc2ccccc23)cc1 ; c1ccc2c(c1)ccc1c(N(c3ccc4ccc5cc6oc7ccccc7c6cc5c4c3)c3ccc4oc5ccccc5c4c3)cccc12 ; c1ccc2c(c1)ccc1ccc(N(c3ccc4ccc5cc6oc7ccccc7c6cc5c4c3)c3ccc4oc5ccccc5c4c3)cc12]

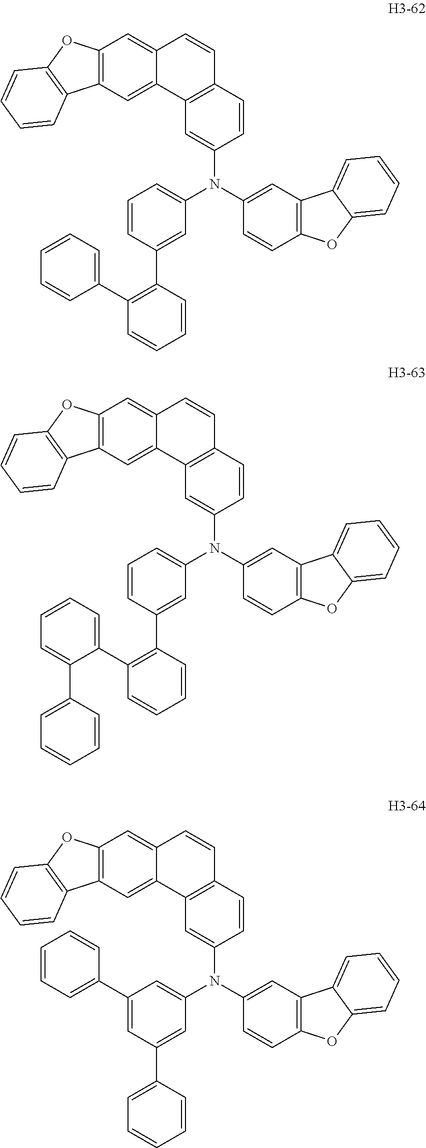 [SMILES: c1ccc(-c2ccccc2-c2cccc(N(c3ccc4ccc5cc6oc7ccccc7c6cc5c4c3)c3ccc4oc5ccccc5c4c3)c2)cc1 ; c1ccc(-c2ccccc2-c2ccccc2-c2cccc(N(c3ccc4ccc5cc6oc7ccccc7c6cc5c4c3)c3ccc4oc5ccccc5c4c3)c2)cc1 ; c1ccc(-c2cc(-c3ccccc3)cc(N(c3ccc4ccc5cc6oc7ccccc7c6cc5c4c3)c3ccc4oc5ccccc5c4c3)c2)cc1]

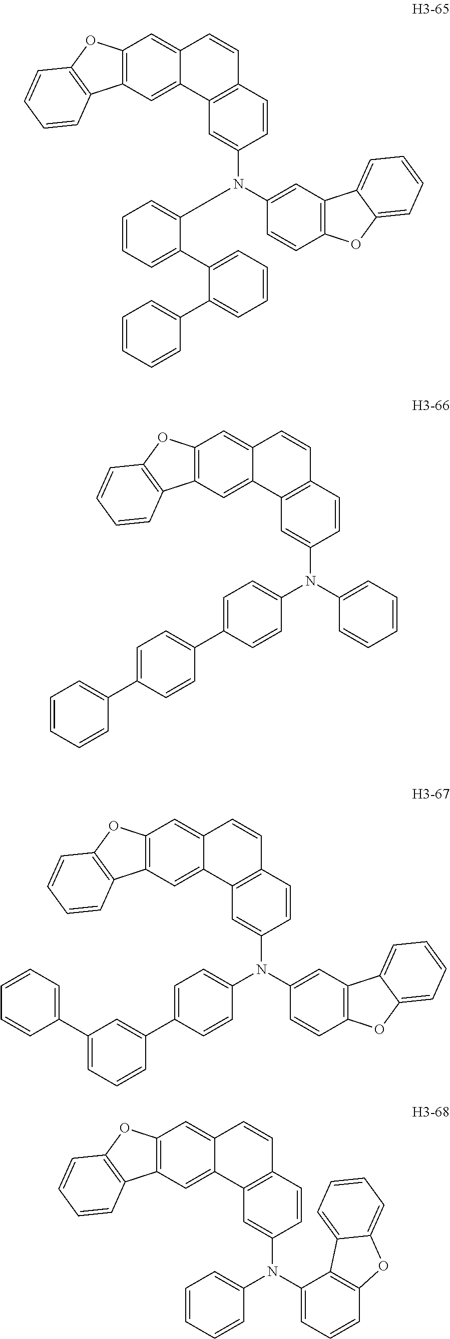

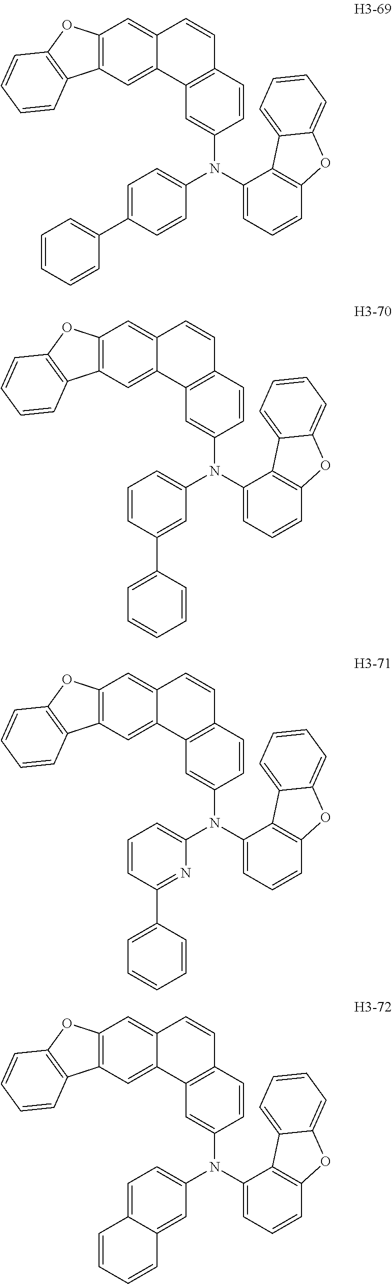

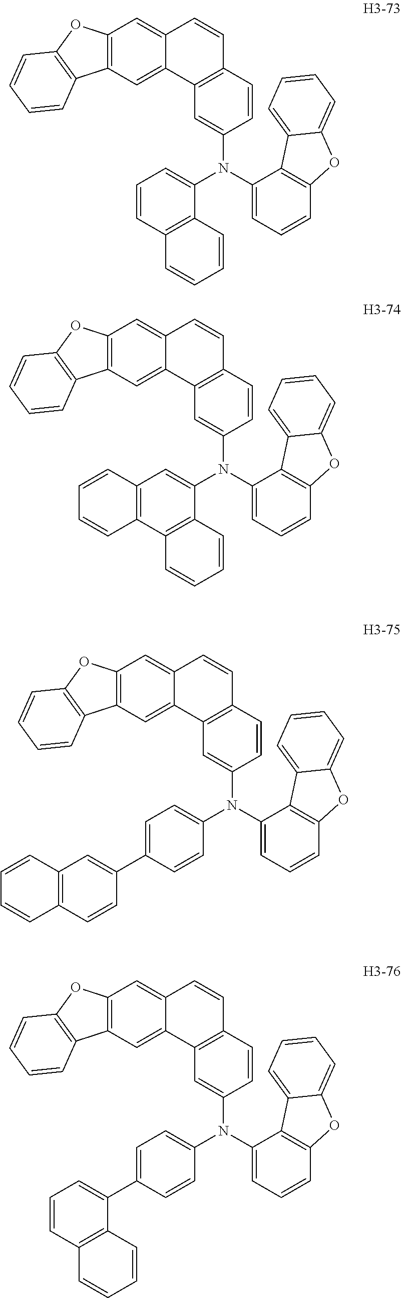

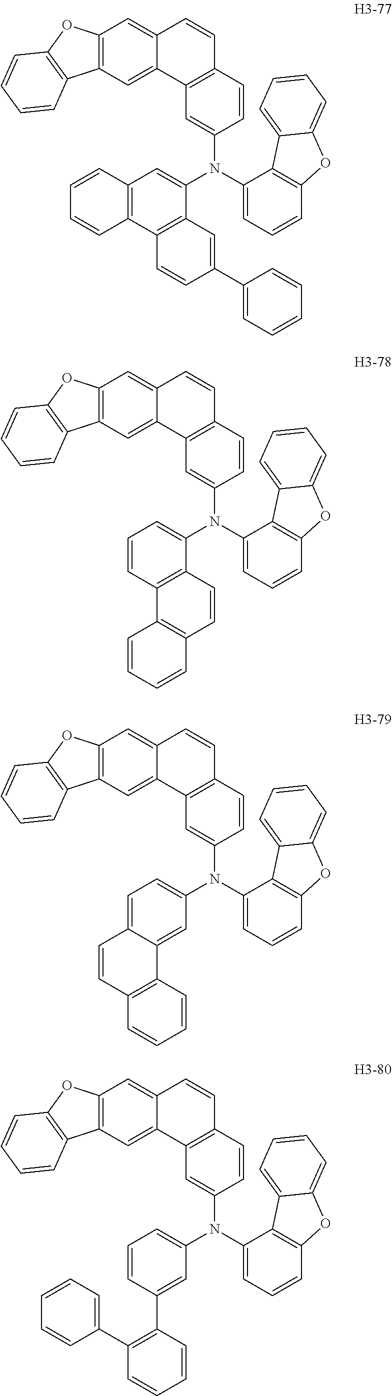

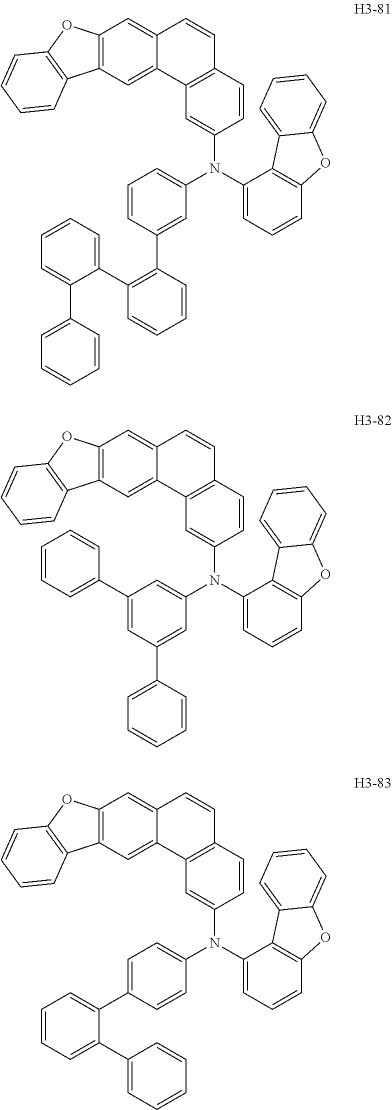 [SMILES: c1ccc(-c2ccccc2-c2ccccc2-c2cccc(N(c3ccc4ccc5cc6oc7ccccc7c6cc5c4c3)c3cccc4oc5ccccc5c34)c2)cc1 ; c1ccc(-c2cc(-c3ccccc3)cc(N(c3ccc4ccc5cc6oc7ccccc7c6cc5c4c3)c3cccc4oc5ccccc5c34)c2)cc1 ; c1ccc(-c2ccccc2-c2ccc(N(c3ccc4ccc5cc6oc7ccccc7c6cc5c4c3)c3cccc4oc5ccccc5c34)cc2)cc1]

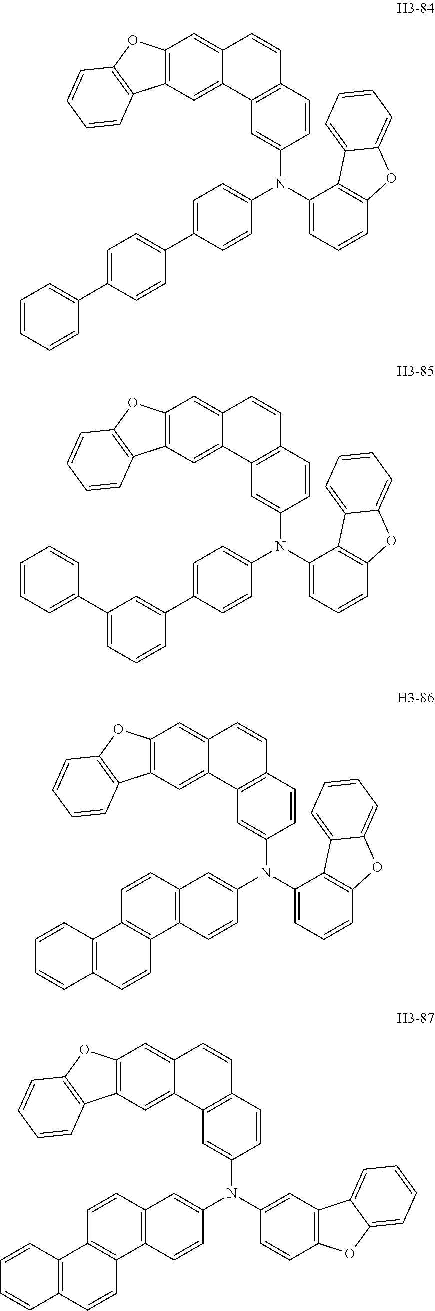 [SMILES: c1ccc(-c2ccc(-c3ccc(N(c4ccc5ccc6cc7oc8ccccc8c7cc6c5c4)c4cccc5oc6ccccc6c45)cc3)cc2)cc1 ; c1ccc(-c2cccc(-c3ccc(N(c4ccc5ccc6cc7oc8ccccc8c7cc6c5c4)c4cccc5oc6ccccc6c45)cc3)c2)cc1 ; c1ccc2c(c1)ccc1c3ccc(N(c4ccc5ccc6cc7oc8ccccc8c7cc6c5c4)c4cccc5oc6ccccc6c45)cc3ccc21 ; c1ccc2c(c1)ccc1c3ccc(N(c4ccc5ccc6cc7oc8ccccc8c7cc6c5c4)c4ccc5oc6ccccc6c5c4)cc3ccc21]

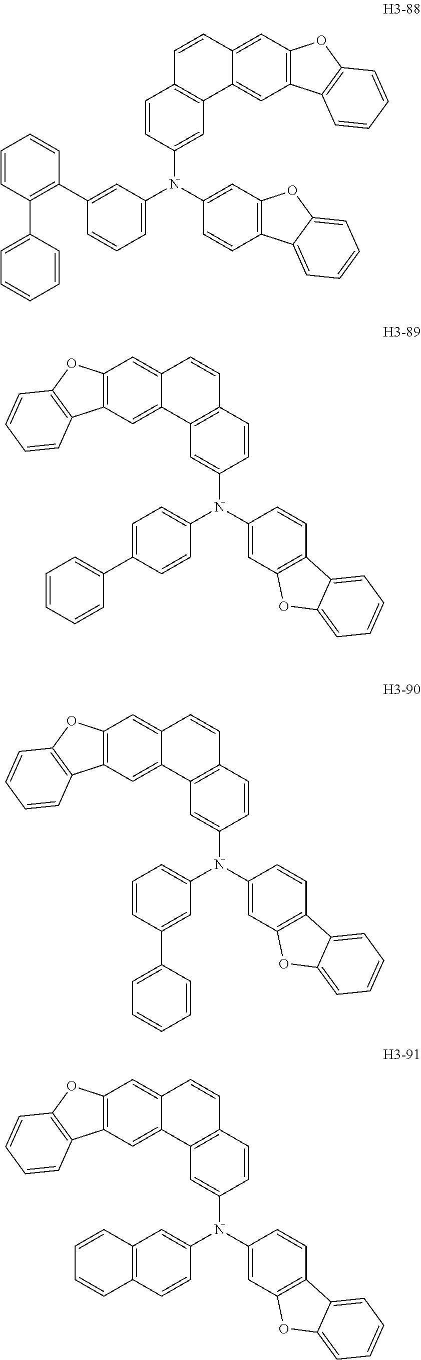 [SMILES: c1ccc(-c2ccccc2-c2cccc(N(c3ccc4c(c3)oc3ccccc34)c3ccc4ccc5cc6oc7ccccc7c6cc5c4c3)c2)cc1 ; c1ccc(-c2ccc(N(c3ccc4c(c3)oc3ccccc34)c3ccc4ccc5cc6oc7ccccc7c6cc5c4c3)cc2)cc1 ; c1ccc(-c2cccc(N(c3ccc4c(c3)oc3ccccc34)c3ccc4ccc5cc6oc7ccccc7c6cc5c4c3)c2)cc1 ; c1ccc2cc(N(c3ccc4c(c3)oc3ccccc34)c3ccc4ccc5cc6oc7ccccc7c6cc5c4c3)ccc2c1]

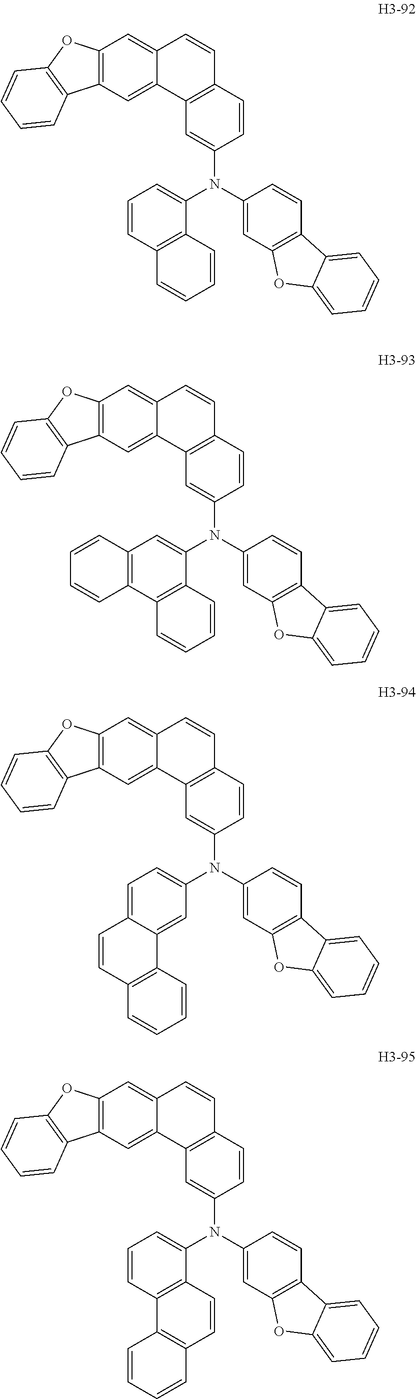 [SMILES: c1ccc2c(N(c3ccc4c(c3)oc3ccccc34)c3ccc4ccc5cc6oc7ccccc7c6cc5c4c3)cccc2c1 ; c1ccc2c(c1)cc(N(c1ccc3c(c1)oc1ccccc13)c1ccc3ccc4cc5oc6ccccc6c5cc4c3c1)c1ccccc12 ; c1ccc2c(c1)ccc1ccc(N(c3ccc4c(c3)oc3ccccc34)c3ccc4ccc5cc6oc7ccccc7c6cc5c4c3)cc12 ; c1ccc2c(c1)ccc1c(N(c3ccc4c(c3)oc3ccccc34)c3ccc4ccc5cc6oc7ccccc7c6cc5c4c3)cccc12]

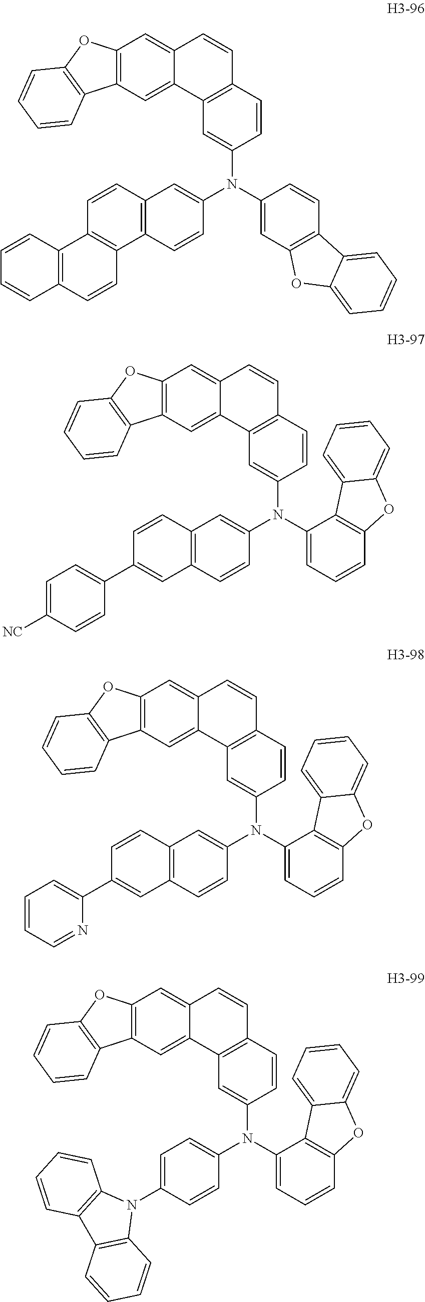 [SMILES: c1ccc2c(c1)ccc1c3ccc(N(c4ccc5c(c4)oc4ccccc45)c4ccc5ccc6cc7oc8ccccc8c7cc6c5c4)cc3ccc21 ; N#Cc1ccc(-c2ccc3cc(N(c4ccc5ccc6cc7oc8ccccc8c7cc6c5c4)c4cccc5oc6ccccc6c45)ccc3c2)cc1 ; c1ccc(-c2ccc3cc(N(c4ccc5ccc6cc7oc8ccccc8c7cc6c5c4)c4cccc5oc6ccccc6c45)ccc3c2)nc1 ; c1ccc2c(c1)oc1cc3ccc4ccc(N(c5ccc(-n6c7ccccc7c7ccccc76)cc5)c5cccc6oc7ccccc7c56)cc4c3cc12]

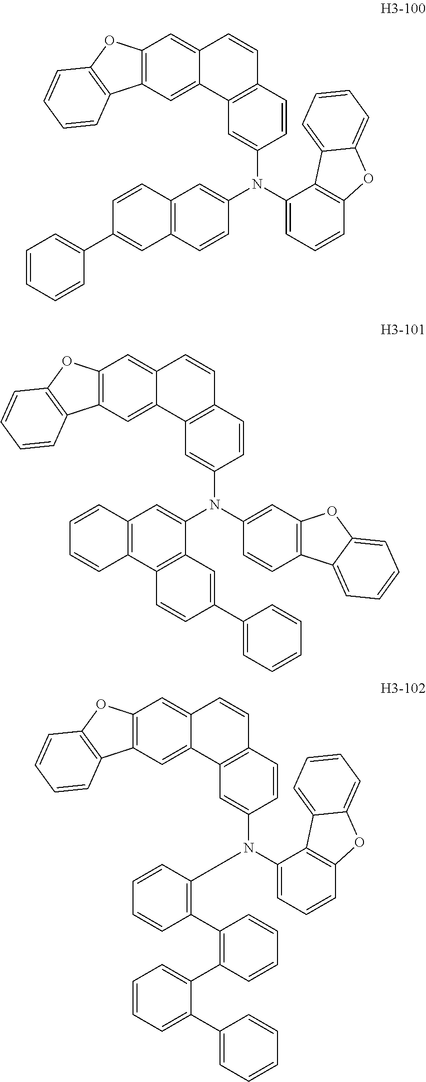 [SMILES: c1ccc(-c2ccc3cc(N(c4ccc5ccc6cc7oc8ccccc8c7cc6c5c4)c4cccc5oc6ccccc6c45)ccc3c2)cc1 ; c1ccc(-c2ccc3c(c2)c(N(c2ccc4c(c2)oc2ccccc24)c2ccc4ccc5cc6oc7ccccc7c6cc5c4c2)cc2ccccc23)cc1 ; c1ccc(-c2ccccc2-c2ccccc2-c2ccccc2CN(c2ccc3ccc4cc5oc6ccccc6c5cc4c3c2)c2cccc3oc4ccccc4c23)cc1]

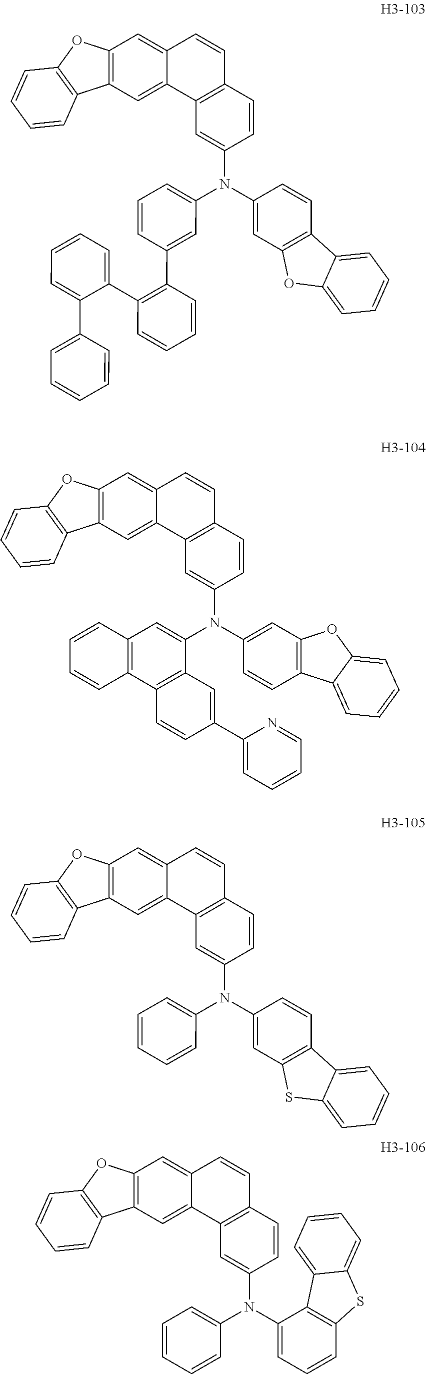 [SMILES: c1ccc(-c2ccccc2-c2ccccc2-c2cccc(N(c3ccc4c(c3)oc3ccccc34)c3ccc4ccc5cc6oc7ccccc7c6cc5c4c3)c2)cc1 ; c1ccc(-c2ccc3c(c2)c(N(c2ccc4c(c2)oc2ccccc24)c2ccc4ccc5cc6oc7ccccc7c6cc5c4c2)cc2ccccc23)nc1 ; c1ccc(N(c2ccc3c(c2)sc2ccccc23)c2ccc3ccc4cc5oc6ccccc6c5cc4c3c2)cc1 ; c1ccc(N(c2ccc3ccc4cc5oc6ccccc6c5cc4c3c2)c2cccc3sc4ccccc4c23)cc1]

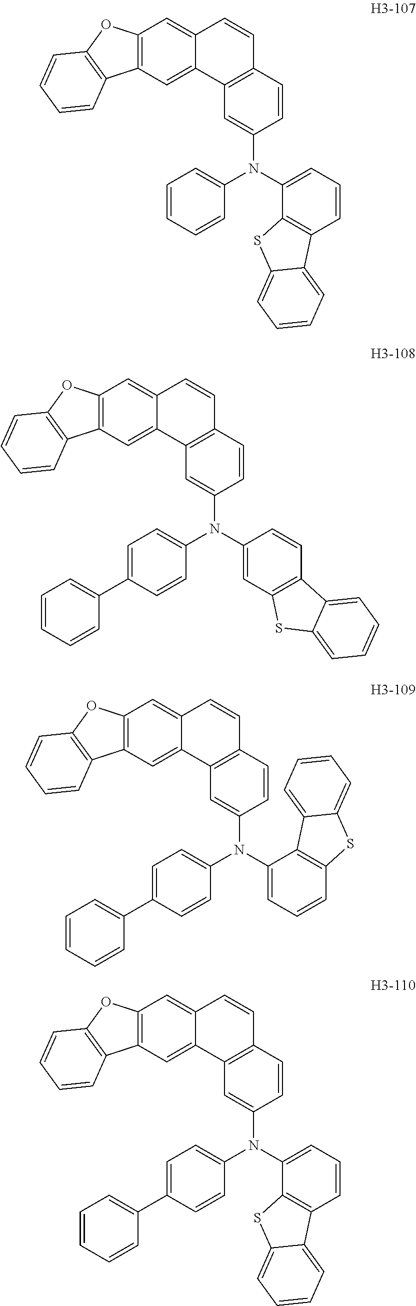

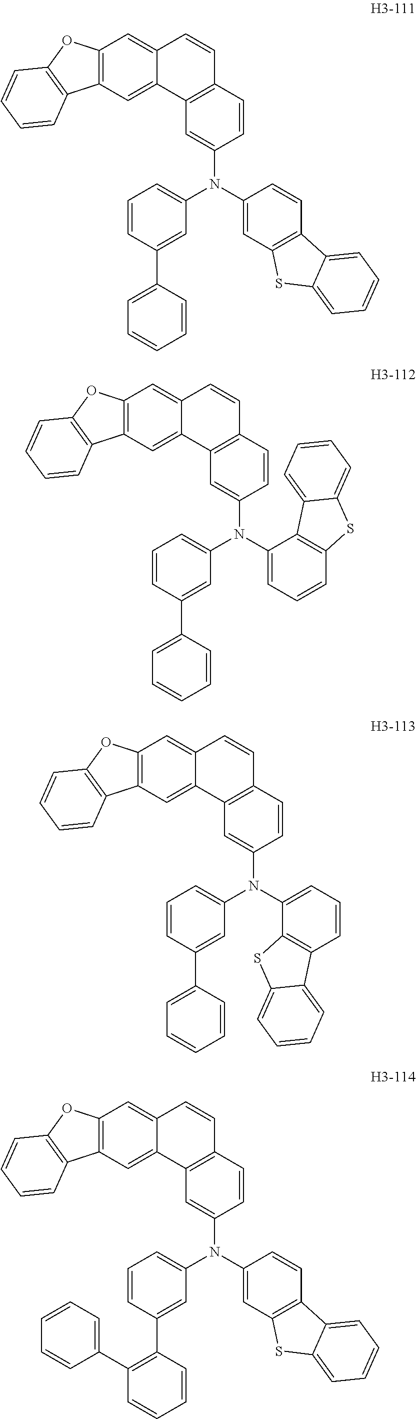

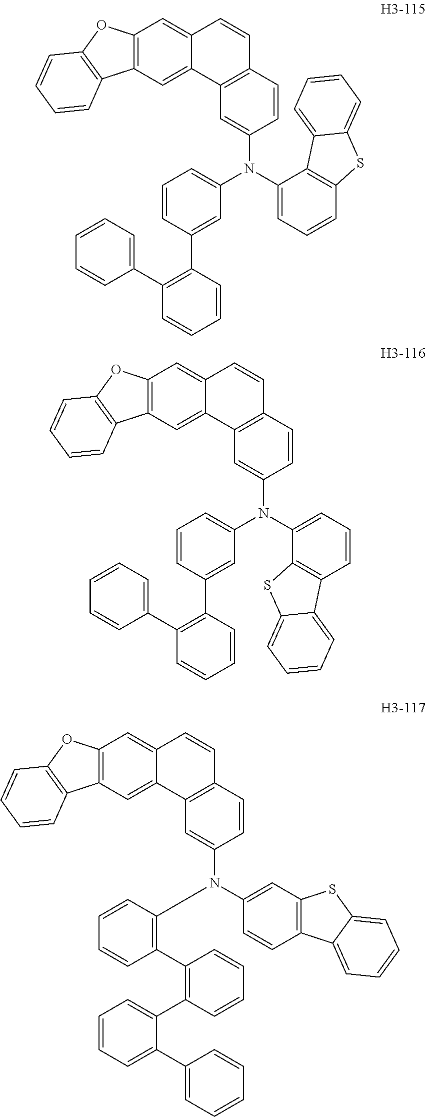

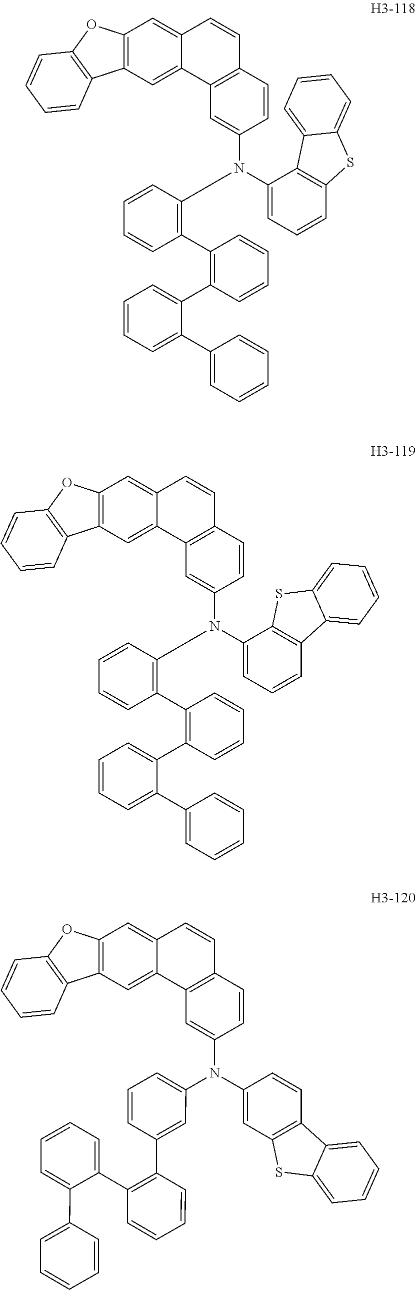 [SMILES: c1ccc(-c2ccccc2-c2ccccc2-c2ccccc2CN(c2ccc3ccc4cc5oc6ccccc6c5cc4c3c2)c2cccc3sc4ccccc4c23)cc1 ; c1ccc(-c2ccccc2-c2ccccc2-c2ccccc2CN(c2ccc3ccc4cc5oc6ccccc6c5cc4c3c2)c2cccc3c2sc2ccccc23)cc1 ; c1ccc(-c2ccccc2-c2ccccc2-c2cccc(N(c3ccc4c(c3)sc3ccccc34)c3ccc4ccc5cc6oc7ccccc7c6cc5c4c3)c2)cc1]

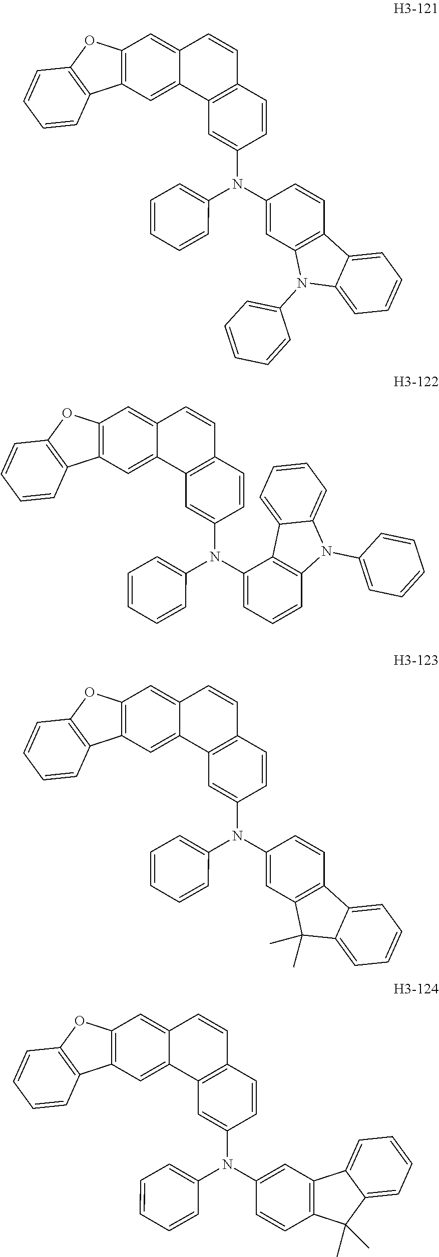 [SMILES: c1ccc(N(c2ccc3ccc4cc5oc6ccccc6c5cc4c3c2)c2ccc3c4ccccc4n(-c4ccccc4)c3c2)cc1 ; c1ccc(N(c2ccc3ccc4cc5oc6ccccc6c5cc4c3c2)c2cccc3c2c2ccccc2n3-c2ccccc2)cc1 ; CC1(C)c2ccccc2-c2ccc(N(c3ccccc3)c3ccc4ccc5cc6oc7ccccc7c6cc5c4c3)cc21 ; CC1(C)c2ccccc2-c2cc(N(c3ccccc3)c3ccc4ccc5cc6oc7ccccc7c6cc5c4c3)ccc21]

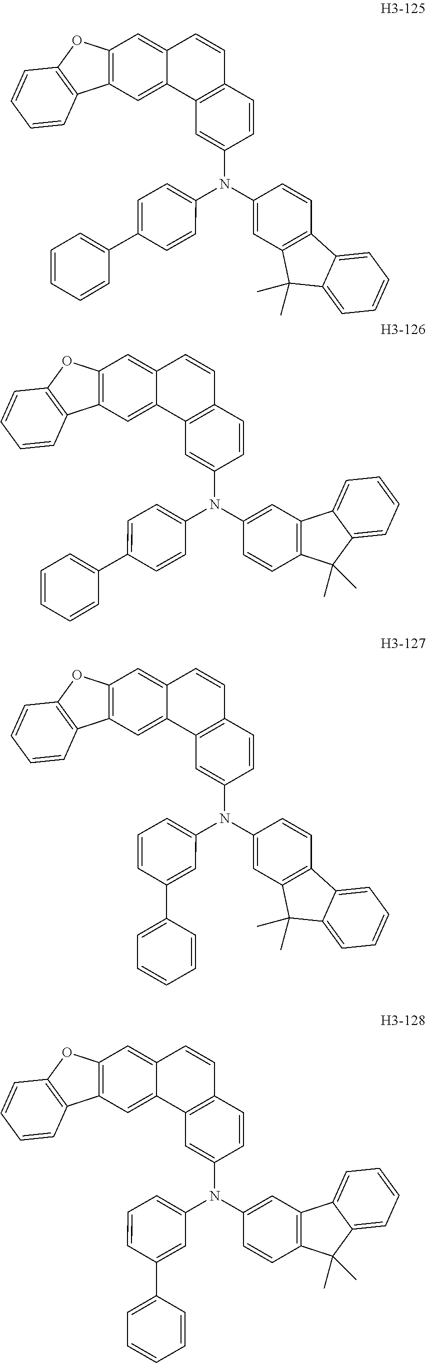 [SMILES: CC1(C)c2ccccc2-c2ccc(N(c3ccc(-c4ccccc4)cc3)c3ccc4ccc5cc6oc7ccccc7c6cc5c4c3)cc21 ; CC1(C)c2ccccc2-c2cc(N(c3ccc(-c4ccccc4)cc3)c3ccc4ccc5cc6oc7ccccc7c6cc5c4c3)ccc21 ; CC1(C)c2ccccc2-c2ccc(N(c3cccc(-c4ccccc4)c3)c3ccc4ccc5cc6oc7ccccc7c6cc5c4c3)cc21 ; CC1(C)c2ccccc2-c2cc(N(c3cccc(-c4ccccc4)c3)c3ccc4ccc5cc6oc7ccccc7c6cc5c4c3)ccc21]

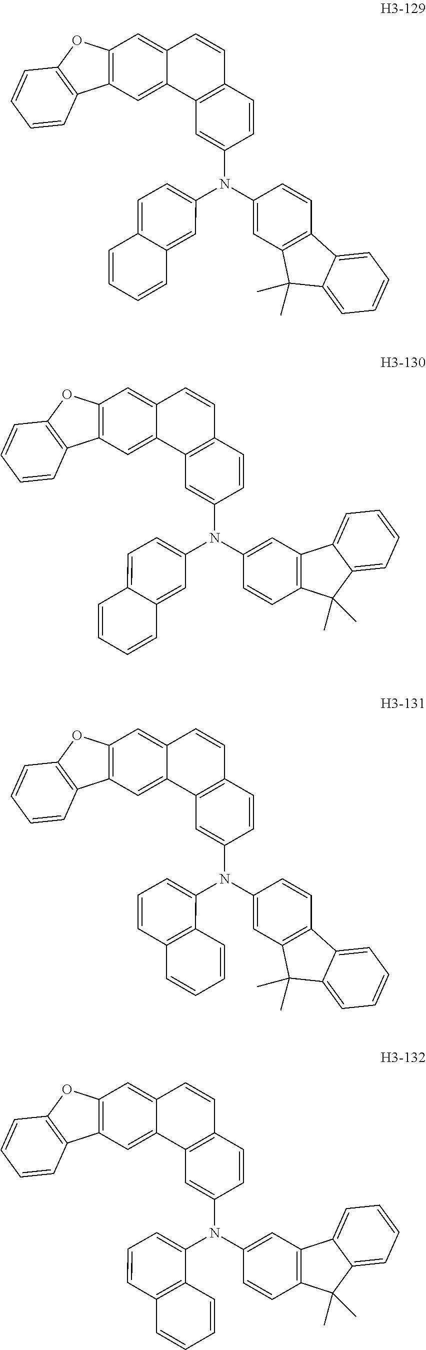 [SMILES: CC1(C)c2ccccc2-c2ccc(N(c3ccc4ccccc4c3)c3ccc4ccc5cc6oc7ccccc7c6cc5c4c3)cc21 ; CC1(C)c2ccccc2-c2cc(N(c3ccc4ccccc4c3)c3ccc4ccc5cc6oc7ccccc7c6cc5c4c3)ccc21 ; CC1(C)c2ccccc2-c2ccc(N(c3ccc4ccc5cc6oc7ccccc7c6cc5c4c3)c3cccc4ccccc34)cc21 ; CC1(C)c2ccccc2-c2cc(N(c3ccc4ccc5cc6oc7ccccc7c6cc5c4c3)c3cccc4ccccc34)ccc21]

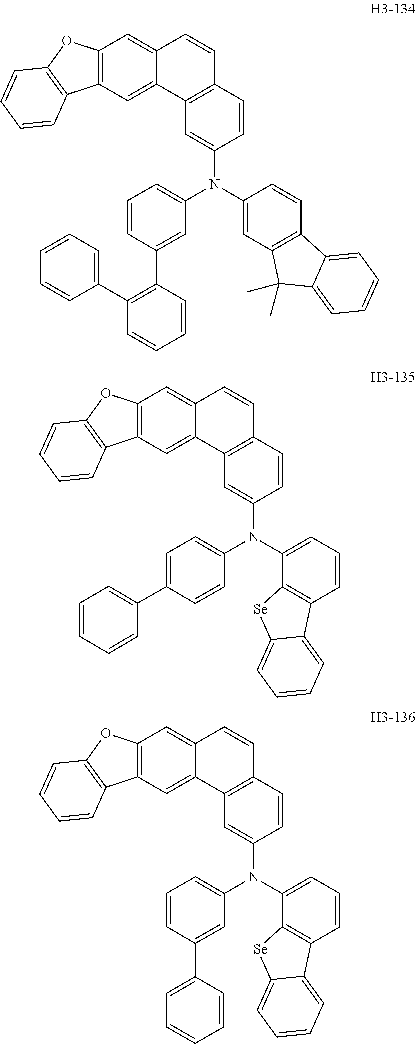

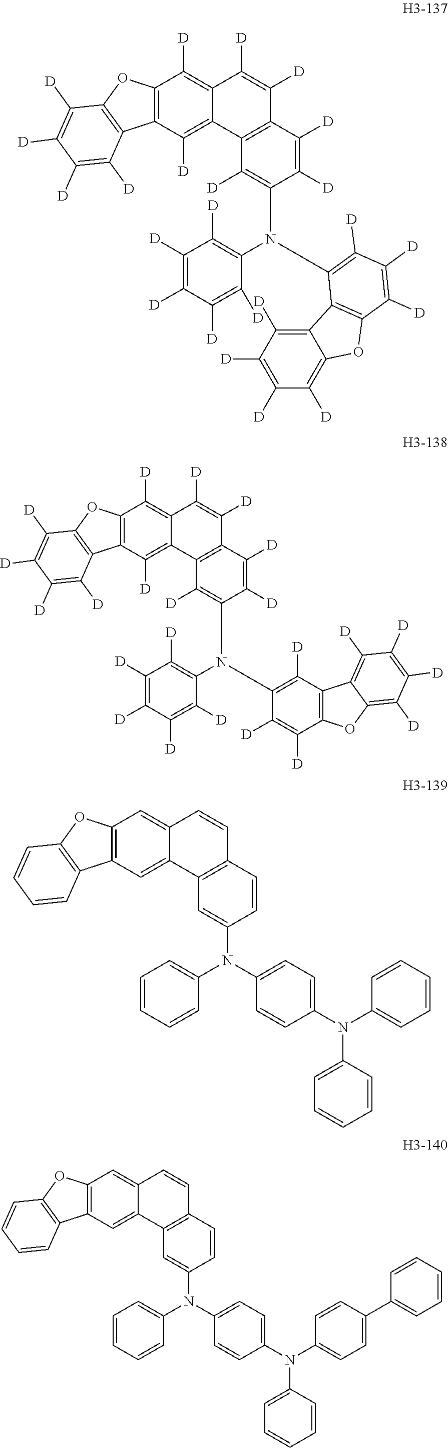

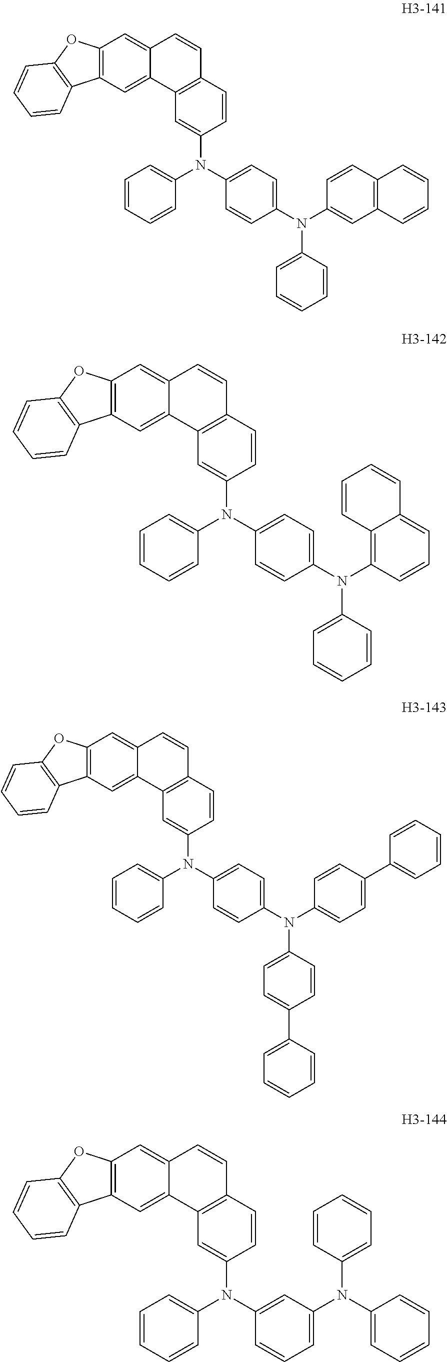 [SMILES: c1ccc(N(c2ccc(N(c3ccccc3)c3ccc4ccc5cc6oc7ccccc7c6cc5c4c3)cc2)c2ccc3ccccc3c2)cc1 ; c1ccc(N(c2ccc(N(c3ccccc3)c3cccc4ccccc34)cc2)c2ccc3ccc4cc5oc6ccccc6c5cc4c3c2)cc1 ; c1ccc(-c2ccc(N(c3ccc(-c4ccccc4)cc3)c3ccc(N(c4ccccc4)c4ccc5ccc6cc7oc8ccccc8c7cc6c5c4)cc3)cc2)cc1 ; c1ccc(N(c2ccccc2)c2cccc(N(c3ccccc3)c3ccc4ccc5cc6oc7ccccc7c6cc5c4c3)c2)cc1]

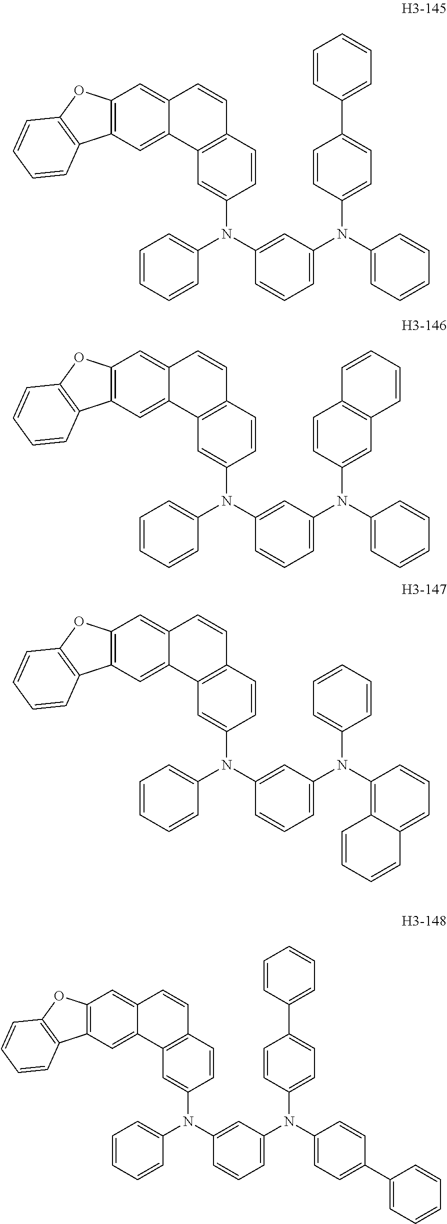 [SMILES: c1ccc(-c2ccc(N(c3ccccc3)c3cccc(N(c4ccccc4)c4ccc5ccc6cc7oc8ccccc8c7cc6c5c4)c3)cc2)cc1 ; c1ccc(N(c2cccc(N(c3ccccc3)c3ccc4ccc5cc6oc7ccccc7c6cc5c4c3)c2)c2ccc3ccccc3c2)cc1 ; c1ccc(N(c2cccc(N(c3ccccc3)c3cccc4ccccc34)c2)c2ccc3ccc4cc5oc6ccccc6c5cc4c3c2)cc1 ; c1ccc(-c2ccc(N(c3ccc(-c4ccccc4)cc3)c3cccc(N(c4ccccc4)c4ccc5ccc6cc7oc8ccccc8c7cc6c5c4)c3)cc2)cc1]

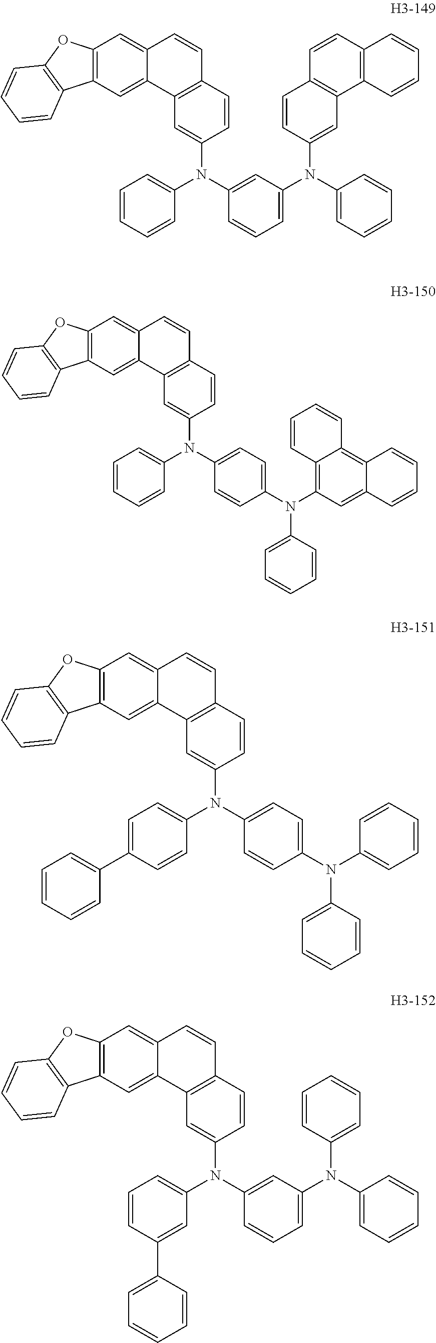 [SMILES: c1ccc(N(c2cccc(N(c3ccccc3)c3ccc4ccc5cc6oc7ccccc7c6cc5c4c3)c2)c2ccc3ccc4ccccc4c3c2)cc1 ; c1ccc(N(c2ccc(N(c3ccccc3)c3cc4ccccc4c4ccccc34)cc2)c2ccc3ccc4cc5oc6ccccc6c5cc4c3c2)cc1 ; c1ccc(-c2ccc(N(c3ccc(N(c4ccccc4)c4ccccc4)cc3)c3ccc4ccc5cc6oc7ccccc7c6cc5c4c3)cc2)cc1 ; c1ccc(-c2cccc(N(c3cccc(N(c4ccccc4)c4ccccc4)c3)c3ccc4ccc5cc6oc7ccccc7c6cc5c4c3)c2)cc1]

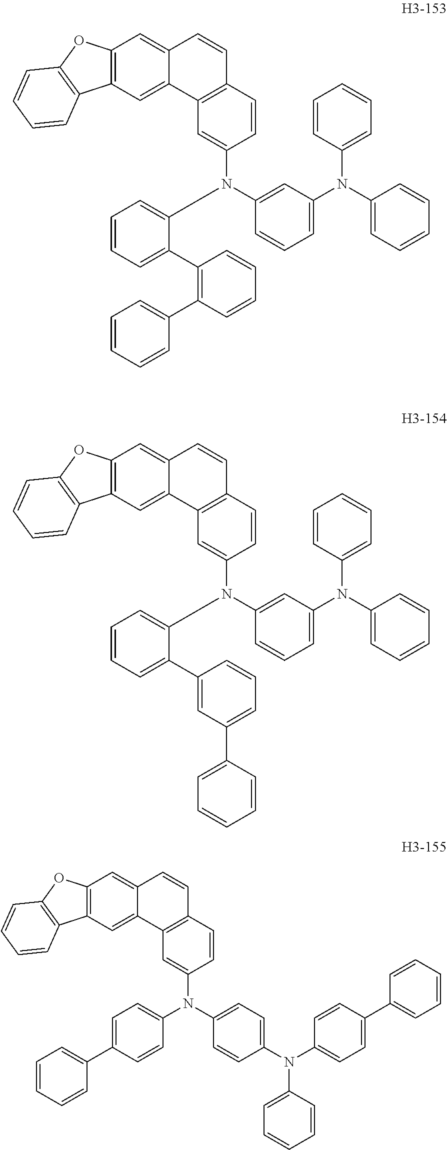 [SMILES: c1ccc(-c2ccccc2-c2ccccc2CN(c2cccc(N(c3ccccc3)c3ccccc3)c2)c2ccc3ccc4cc5oc6ccccc6c5cc4c3c2)cc1 ; c1ccc(-c2cccc(-c3ccccc3CN(c3cccc(N(c4ccccc4)c4ccccc4)c3)c3ccc4ccc5cc6oc7ccccc7c6cc5c4c3)c2)cc1 ; c1ccc(-c2ccc(N(c3ccccc3)c3ccc(N(c4ccc(-c5ccccc5)cc4)c4ccc5ccc6cc7oc8ccccc8c7cc6c5c4)cc3)cc2)cc1]

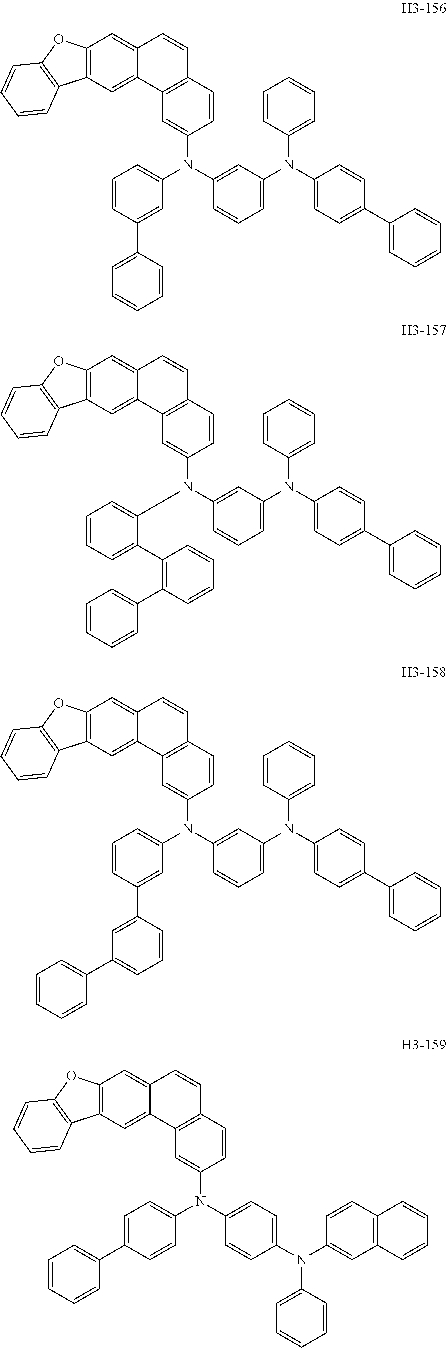 [SMILES: c1ccc(-c2ccc(N(c3ccccc3)c3cccc(N(c4cccc(-c5ccccc5)c4)c4ccc5ccc6cc7oc8ccccc8c7cc6c5c4)c3)cc2)cc1 ; c1ccc(-c2ccc(N(c3ccccc3)c3cccc(N(Cc4ccccc4-c4ccccc4-c4ccccc4)c4ccc5ccc6cc7oc8ccccc8c7cc6c5c4)c3)cc2)cc1 ; c1ccc(-c2ccc(N(c3ccccc3)c3cccc(N(c4cccc(-c5cccc(-c6ccccc6)c5)c4)c4ccc5ccc6cc7oc8ccccc8c7cc6c5c4)c3)cc2)cc1 ; c1ccc(-c2ccc(N(c3ccc(N(c4ccccc4)c4ccc5ccccc5c4)cc3)c3ccc4ccc5cc6oc7ccccc7c6cc5c4c3)cc2)cc1]

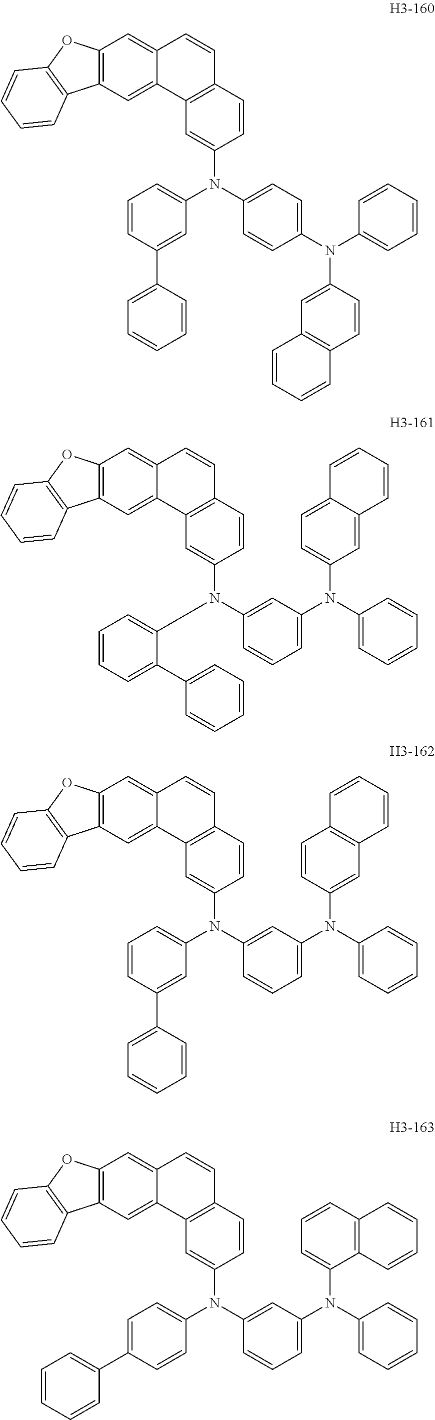 [SMILES: c1ccc(-c2cccc(N(c3ccc(N(c4ccccc4)c4ccc5ccccc5c4)cc3)c3ccc4ccc5cc6oc7ccccc7c6cc5c4c3)c2)cc1 ; c1ccc(-c2ccccc2CN(c2cccc(N(c3ccccc3)c3ccc4ccccc4c3)c2)c2ccc3ccc4cc5oc6ccccc6c5cc4c3c2)cc1 ; c1ccc(-c2cccc(N(c3cccc(N(c4ccccc4)c4ccc5ccccc5c4)c3)c3ccc4ccc5cc6oc7ccccc7c6cc5c4c3)c2)cc1 ; c1ccc(-c2ccc(N(c3cccc(N(c4ccccc4)c4cccc5ccccc45)c3)c3ccc4ccc5cc6oc7ccccc7c6cc5c4c3)cc2)cc1]

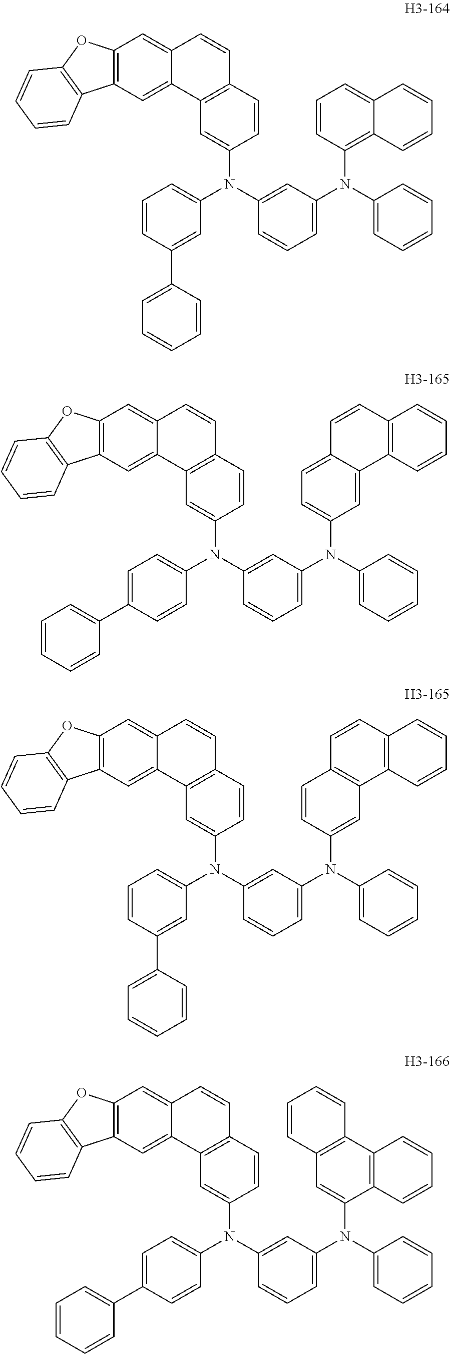 [SMILES: c1ccc(-c2cccc(N(c3cccc(N(c4ccccc4)c4cccc5ccccc45)c3)c3ccc4ccc5cc6oc7ccccc7c6cc5c4c3)c2)cc1 ; c1ccc(-c2ccc(N(c3cccc(N(c4ccccc4)c4ccc5ccc6ccccc6c5c4)c3)c3ccc4ccc5cc6oc7ccccc7c6cc5c4c3)cc2)cc1 ; c1ccc(-c2cccc(N(c3cccc(N(c4ccccc4)c4ccc5ccc6ccccc6c5c4)c3)c3ccc4ccc5cc6oc7ccccc7c6cc5c4c3)c2)cc1 ; c1ccc(-c2ccc(N(c3cccc(N(c4ccccc4)c4cc5ccccc5c5ccccc45)c3)c3ccc4ccc5cc6oc7ccccc7c6cc5c4c3)cc2)cc1]

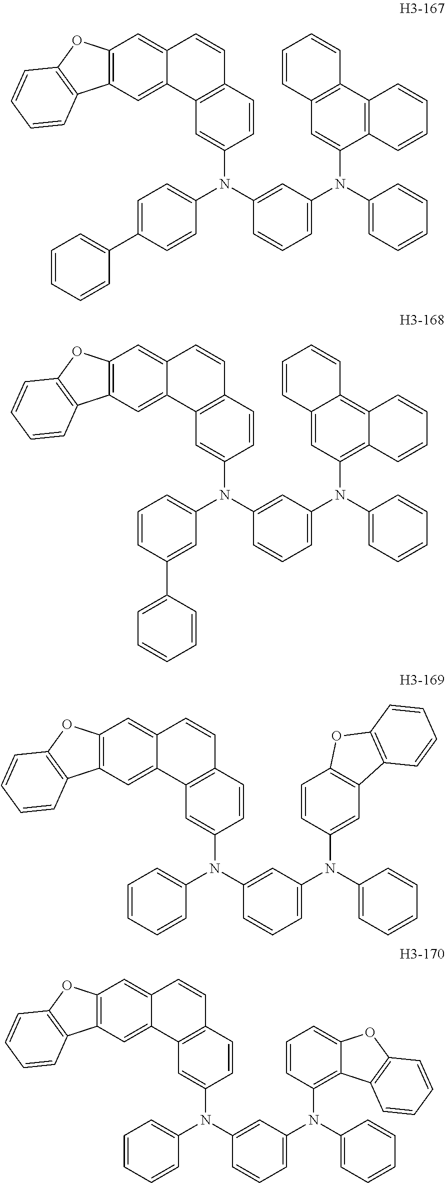 [SMILES: c1ccc(-c2ccc(N(c3cccc(N(c4ccccc4)c4cc5ccccc5c5ccccc45)c3)c3ccc4ccc5cc6oc7ccccc7c6cc5c4c3)cc2)cc1 ; c1ccc(-c2cccc(N(c3cccc(N(c4ccccc4)c4cc5ccccc5c5ccccc45)c3)c3ccc4ccc5cc6oc7ccccc7c6cc5c4c3)c2)cc1 ; c1ccc(N(c2cccc(N(c3ccccc3)c3ccc4oc5ccccc5c4c3)c2)c2ccc3ccc4cc5oc6ccccc6c5cc4c3c2)cc1 ; c1ccc(N(c2cccc(N(c3ccccc3)c3cccc4oc5ccccc5c34)c2)c2ccc3ccc4cc5oc6ccccc6c5cc4c3c2)cc1]

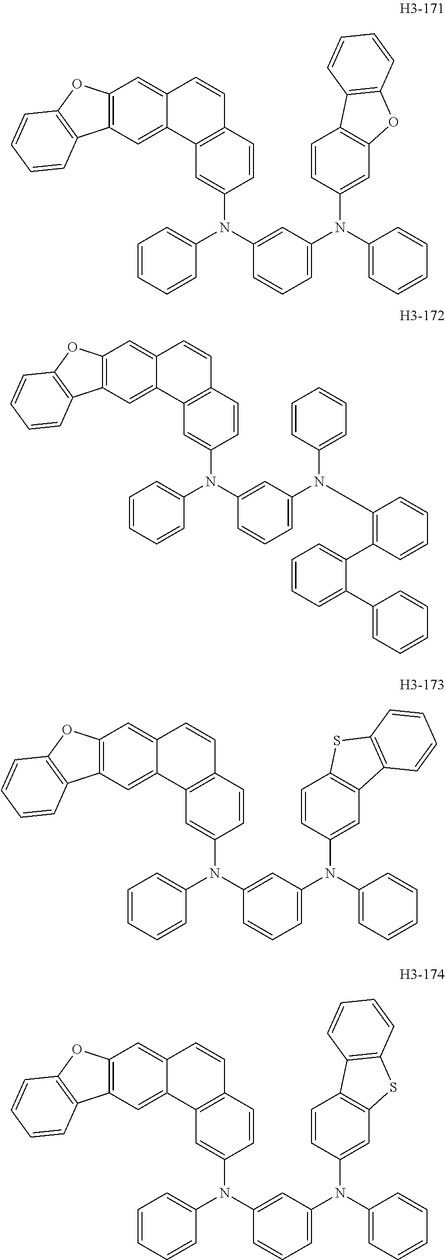 [SMILES: c1ccc(N(c2cccc(N(c3ccccc3)c3ccc4ccc5cc6oc7ccccc7c6cc5c4c3)c2)c2ccc3c(c2)oc2ccccc23)cc1 ; c1ccc(-c2ccccc2-c2ccccc2CN(c2ccccc2)c2cccc(N(c3ccccc3)c3ccc4ccc5cc6oc7ccccc7c6cc5c4c3)c2)cc1 ; c1ccc(N(c2cccc(N(c3ccccc3)c3ccc4sc5ccccc5c4c3)c2)c2ccc3ccc4cc5oc6ccccc6c5cc4c3c2)cc1 ; c1ccc(N(c2cccc(N(c3ccccc3)c3ccc4ccc5cc6oc7ccccc7c6cc5c4c3)c2)c2ccc3c(c2)sc2ccccc23)cc1]

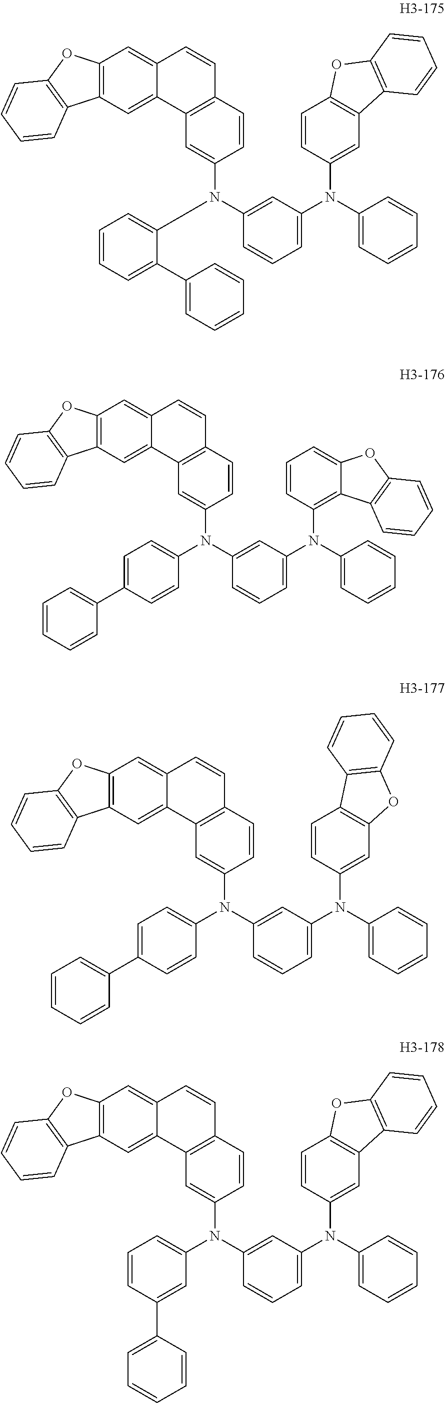 [SMILES: c1ccc(-c2ccccc2CN(c2cccc(N(c3ccccc3)c3ccc4oc5ccccc5c4c3)c2)c2ccc3ccc4cc5oc6ccccc6c5cc4c3c2)cc1 ; c1ccc(-c2ccc(N(c3cccc(N(c4ccccc4)c4cccc5oc6ccccc6c45)c3)c3ccc4ccc5cc6oc7ccccc7c6cc5c4c3)cc2)cc1 ; c1ccc(-c2ccc(N(c3cccc(N(c4ccccc4)c4ccc5c(c4)oc4ccccc45)c3)c3ccc4ccc5cc6oc7ccccc7c6cc5c4c3)cc2)cc1 ; c1ccc(-c2cccc(N(c3cccc(N(c4ccccc4)c4ccc5oc6ccccc6c5c4)c3)c3ccc4ccc5cc6oc7ccccc7c6cc5c4c3)c2)cc1]

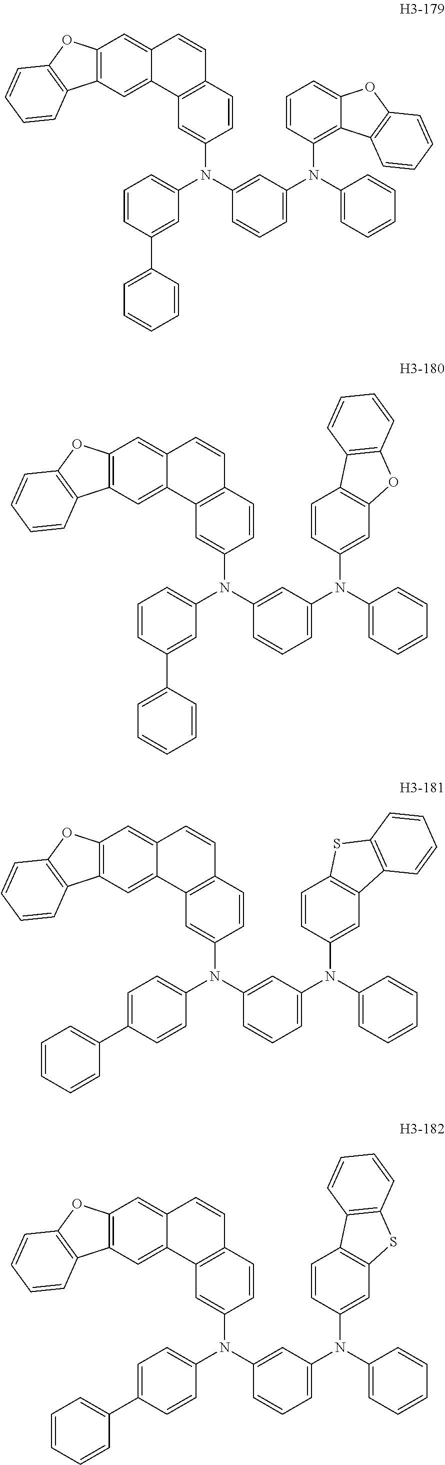 [SMILES: c1ccc(-c2cccc(N(c3cccc(N(c4ccccc4)c4cccc5oc6ccccc6c45)c3)c3ccc4ccc5cc6oc7ccccc7c6cc5c4c3)c2)cc1 ; c1ccc(-c2cccc(N(c3cccc(N(c4ccccc4)c4ccc5c(c4)oc4ccccc45)c3)c3ccc4ccc5cc6oc7ccccc7c6cc5c4c3)c2)cc1 ; c1ccc(-c2ccc(N(c3cccc(N(c4ccccc4)c4ccc5sc6ccccc6c5c4)c3)c3ccc4ccc5cc6oc7ccccc7c6cc5c4c3)cc2)cc1 ; c1ccc(-c2ccc(N(c3cccc(N(c4ccccc4)c4ccc5c(c4)sc4ccccc45)c3)c3ccc4ccc5cc6oc7ccccc7c6cc5c4c3)cc2)cc1]

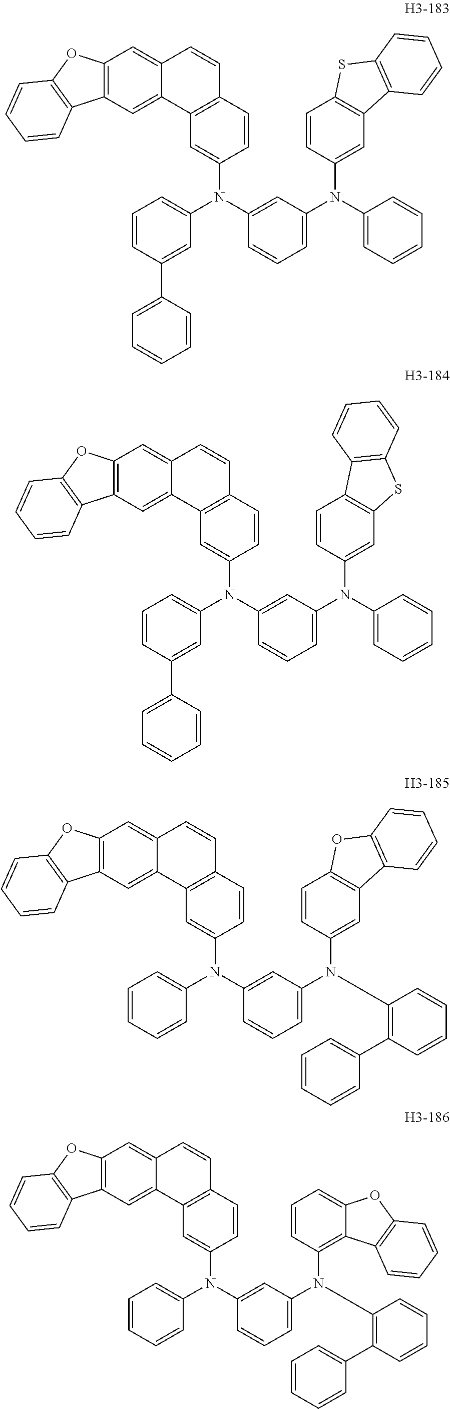 [SMILES: c1ccc(-c2cccc(N(c3cccc(N(c4ccccc4)c4ccc5sc6ccccc6c5c4)c3)c3ccc4ccc5cc6oc7ccccc7c6cc5c4c3)c2)cc1 ; c1ccc(-c2cccc(N(c3cccc(N(c4ccccc4)c4ccc5c(c4)sc4ccccc45)c3)c3ccc4ccc5cc6oc7ccccc7c6cc5c4c3)c2)cc1 ; c1ccc(-c2ccccc2CN(c2cccc(N(c3ccccc3)c3ccc4ccc5cc6oc7ccccc7c6cc5c4c3)c2)c2ccc3oc4ccccc4c3c2)cc1 ; c1ccc(-c2ccccc2CN(c2cccc(N(c3ccccc3)c3ccc4ccc5cc6oc7ccccc7c6cc5c4c3)c2)c2cccc3oc4ccccc4c23)cc1]

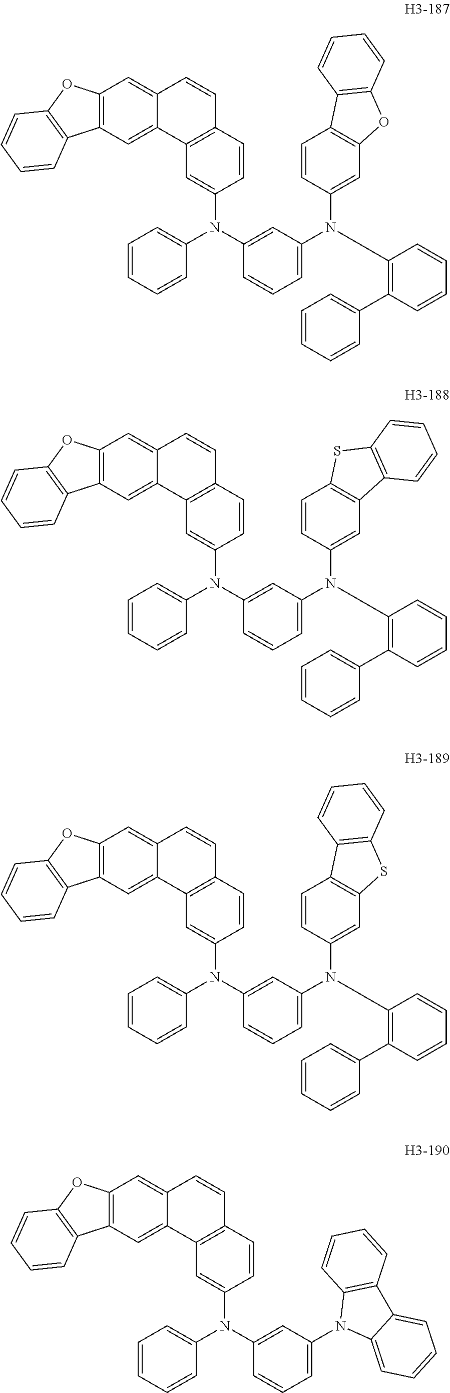 [SMILES: c1ccc(-c2ccccc2CN(c2cccc(N(c3ccccc3)c3ccc4ccc5cc6oc7ccccc7c6cc5c4c3)c2)c2ccc3c(c2)oc2ccccc23)cc1 ; c1ccc(-c2ccccc2CN(c2cccc(N(c3ccccc3)c3ccc4ccc5cc6oc7ccccc7c6cc5c4c3)c2)c2ccc3sc4ccccc4c3c2)cc1 ; c1ccc(-c2ccccc2CN(c2cccc(N(c3ccccc3)c3ccc4ccc5cc6oc7ccccc7c6cc5c4c3)c2)c2ccc3c(c2)sc2ccccc23)cc1 ; c1ccc(N(c2cccc(-n3c4ccccc4c4ccccc43)c2)c2ccc3ccc4cc5oc6ccccc6c5cc4c3c2)cc1]

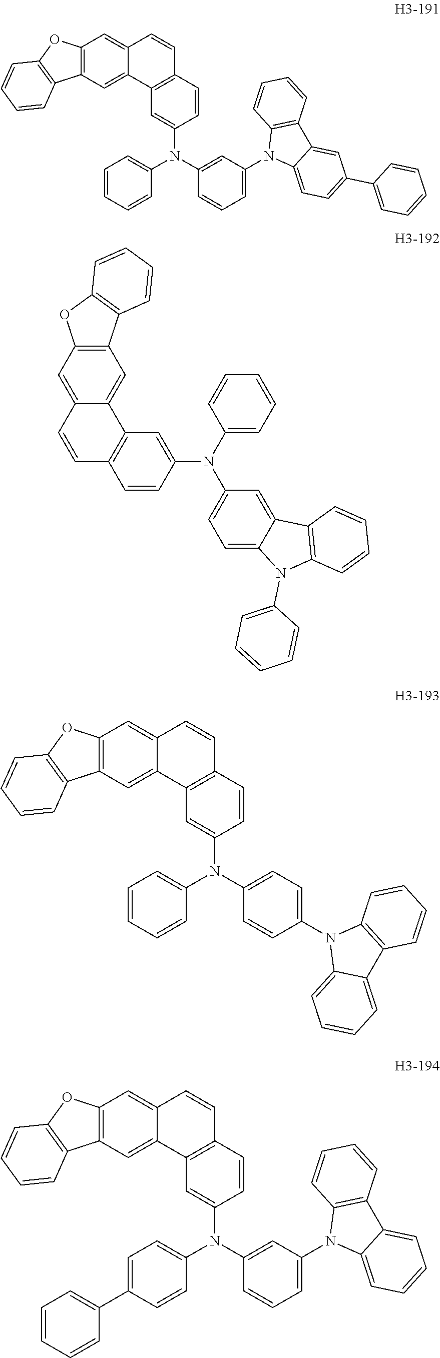

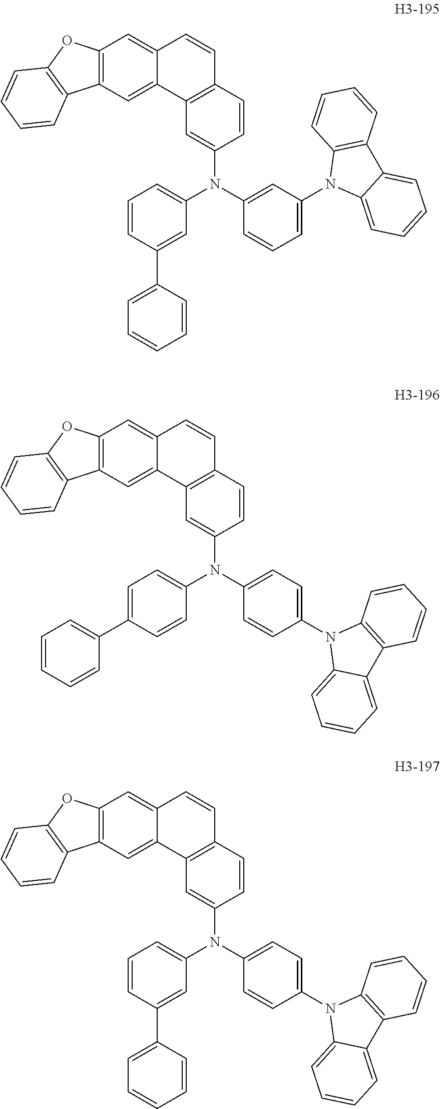 [SMILES: c1ccc(-c2cccc(N(c3cccc(-n4c5ccccc5c5ccccc54)c3)c3ccc4ccc5cc6oc7ccccc7c6cc5c4c3)c2)cc1 ; c1ccc(-c2ccc(N(c3ccc(-n4c5ccccc5c5ccccc54)cc3)c3ccc4ccc5cc6oc7ccccc7c6cc5c4c3)cc2)cc1 ; c1ccc(-c2cccc(N(c3ccc(-n4c5ccccc5c5ccccc54)cc3)c3ccc4ccc5cc6oc7ccccc7c6cc5c4c3)c2)cc1]

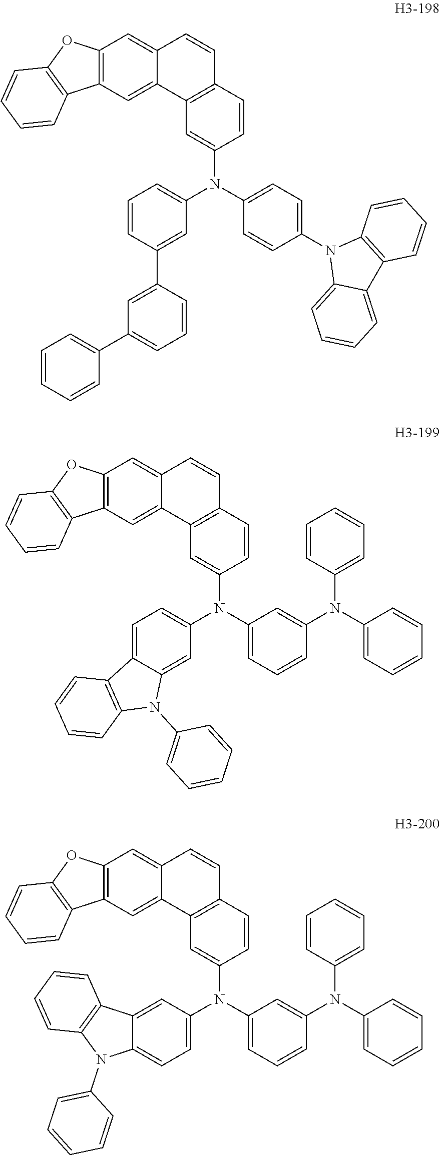 [SMILES: c1ccc(-c2cccc(-c3cccc(N(c4ccc(-n5c6ccccc6c6ccccc65)cc4)c4ccc5ccc6cc7oc8ccccc8c7cc6c5c4)c3)c2)cc1 ; c1ccc(N(c2ccccc2)c2cccc(N(c3ccc4ccc5cc6oc7ccccc7c6cc5c4c3)c3ccc4c5ccccc5n(-c5ccccc5)c4c3)c2)cc1 ; c1ccc(N(c2ccccc2)c2cccc(N(c3ccc4ccc5cc6oc7ccccc7c6cc5c4c3)c3ccc4c(c3)c3ccccc3n4-c3ccccc3)c2)cc1]

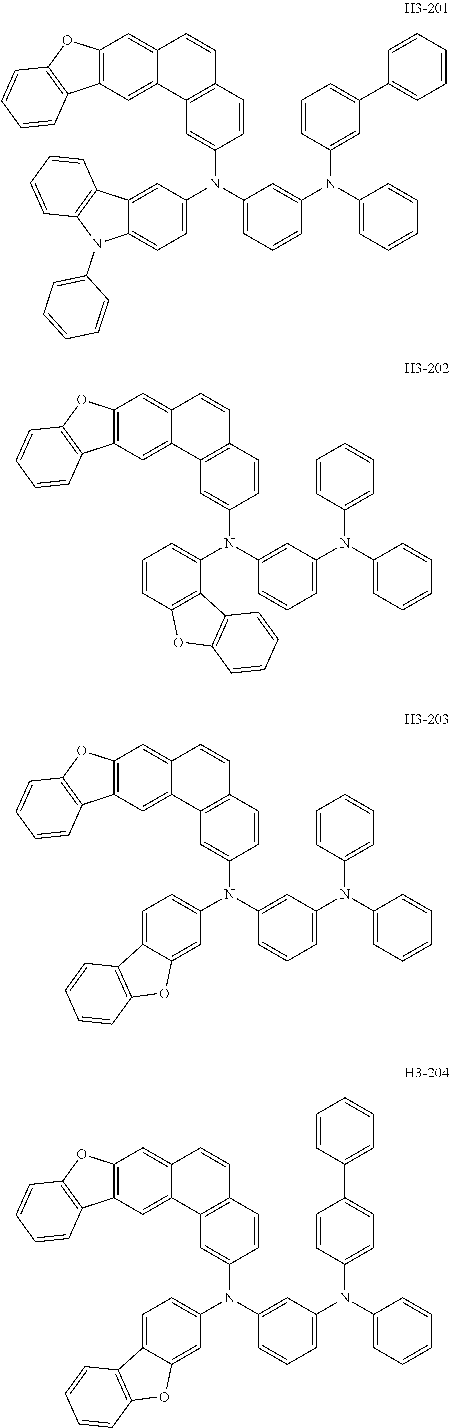 [SMILES: c1ccc(-c2cccc(N(c3ccccc3)c3cccc(N(c4ccc5ccc6cc7oc8ccccc8c7cc6c5c4)c4ccc5c(c4)c4ccccc4n5-c4ccccc4)c3)c2)cc1 ; c1ccc(N(c2ccccc2)c2cccc(N(c3ccc4ccc5cc6oc7ccccc7c6cc5c4c3)c3cccc4oc5ccccc5c34)c2)cc1 ; c1ccc(N(c2ccccc2)c2cccc(N(c3ccc4c(c3)oc3ccccc34)c3ccc4ccc5cc6oc7ccccc7c6cc5c4c3)c2)cc1 ; c1ccc(-c2ccc(N(c3ccccc3)c3cccc(N(c4ccc5c(c4)oc4ccccc45)c4ccc5ccc6cc7oc8ccccc8c7cc6c5c4)c3)cc2)cc1]

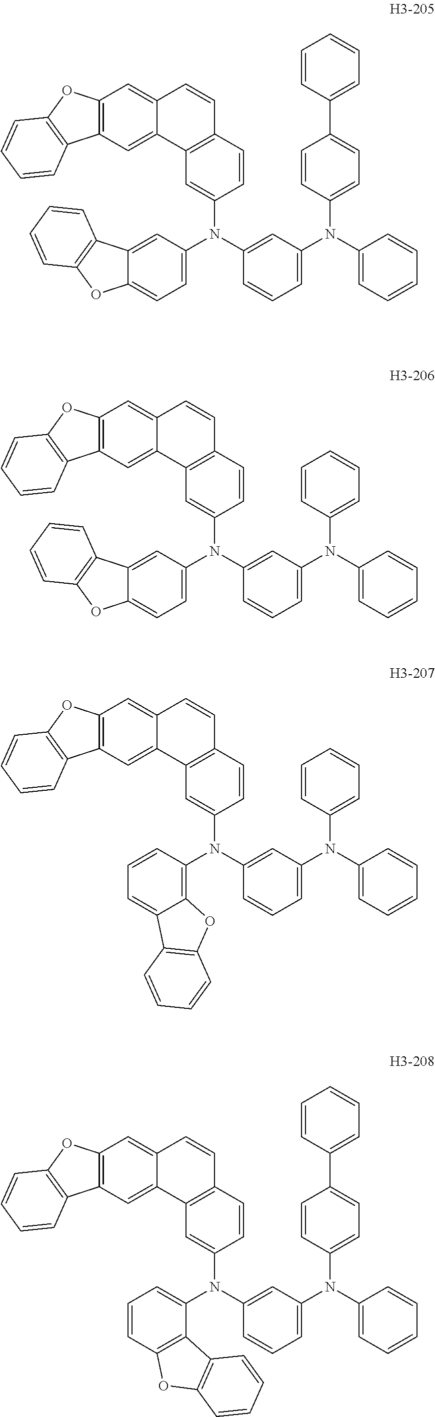 [SMILES: c1ccc(-c2ccc(N(c3ccccc3)c3cccc(N(c4ccc5ccc6cc7oc8ccccc8c7cc6c5c4)c4ccc5oc6ccccc6c5c4)c3)cc2)cc1 ; c1ccc(N(c2ccccc2)c2cccc(N(c3ccc4ccc5cc6oc7ccccc7c6cc5c4c3)c3ccc4oc5ccccc5c4c3)c2)cc1 ; c1ccc(N(c2ccccc2)c2cccc(N(c3ccc4ccc5cc6oc7ccccc7c6cc5c4c3)c3cccc4c3oc3ccccc34)c2)cc1 ; c1ccc(-c2ccc(N(c3ccccc3)c3cccc(N(c4ccc5ccc6cc7oc8ccccc8c7cc6c5c4)c4cccc5oc6ccccc6c45)c3)cc2)cc1]

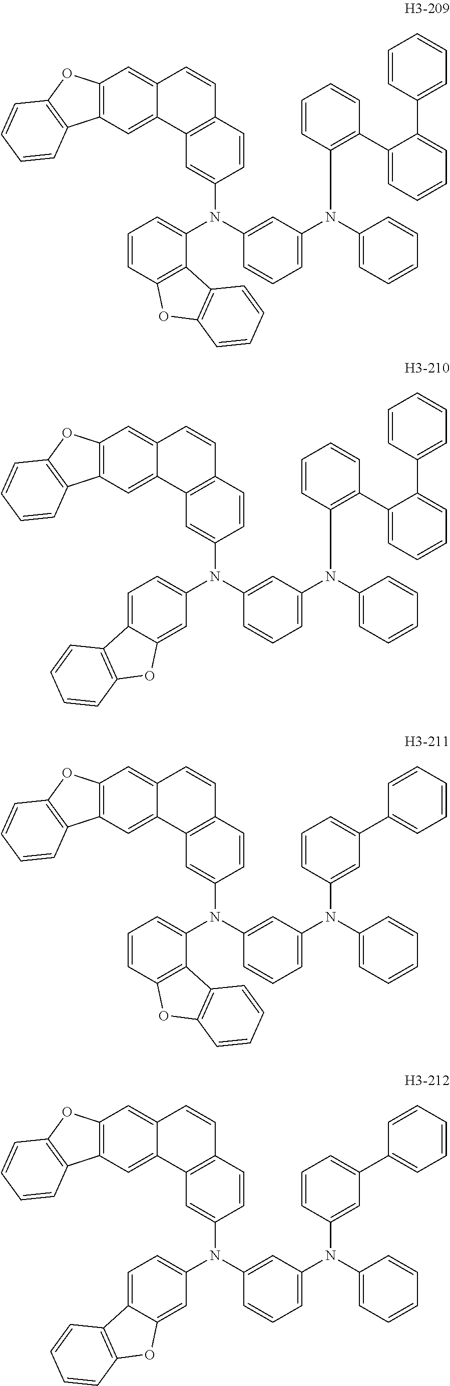 [SMILES: c1ccc(-c2ccccc2-c2ccccc2CN(c2ccccc2)c2cccc(N(c3ccc4ccc5cc6oc7ccccc7c6cc5c4c3)c3cccc4oc5ccccc5c34)c2)cc1 ; c1ccc(-c2ccccc2-c2ccccc2CN(c2ccccc2)c2cccc(N(c3ccc4c(c3)oc3ccccc34)c3ccc4ccc5cc6oc7ccccc7c6cc5c4c3)c2)cc1 ; c1ccc(-c2cccc(N(c3ccccc3)c3cccc(N(c4ccc5ccc6cc7oc8ccccc8c7cc6c5c4)c4cccc5oc6ccccc6c45)c3)c2)cc1 ; c1ccc(-c2cccc(N(c3ccccc3)c3cccc(N(c4ccc5c(c4)oc4ccccc45)c4ccc5ccc6cc7oc8ccccc8c7cc6c5c4)c3)c2)cc1]

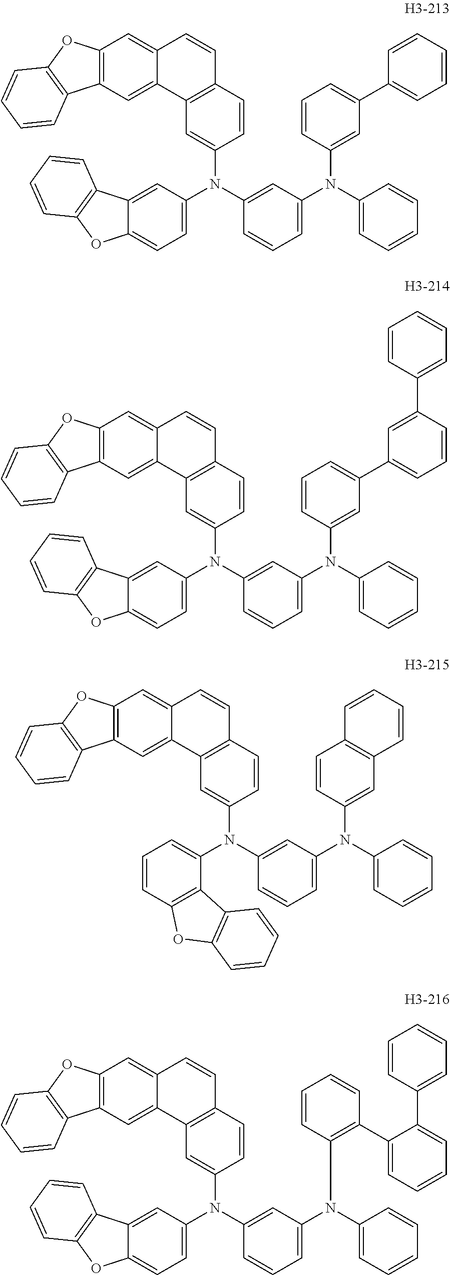 [SMILES: c1ccc(-c2cccc(N(c3ccccc3)c3cccc(N(c4ccc5ccc6cc7oc8ccccc8c7cc6c5c4)c4ccc5oc6ccccc6c5c4)c3)c2)cc1 ; c1ccc(-c2cccc(-c3cccc(N(c4ccccc4)c4cccc(N(c5ccc6ccc7cc8oc9ccccc9c8cc7c6c5)c5ccc6oc7ccccc7c6c5)c4)c3)c2)cc1 ; c1ccc(N(c2cccc(N(c3ccc4ccc5cc6oc7ccccc7c6cc5c4c3)c3cccc4oc5ccccc5c34)c2)c2ccc3ccccc3c2)cc1 ; c1ccc(-c2ccccc2-c2ccccc2CN(c2ccccc2)c2cccc(N(c3ccc4ccc5cc6oc7ccccc7c6cc5c4c3)c3ccc4oc5ccccc5c4c3)c2)cc1]

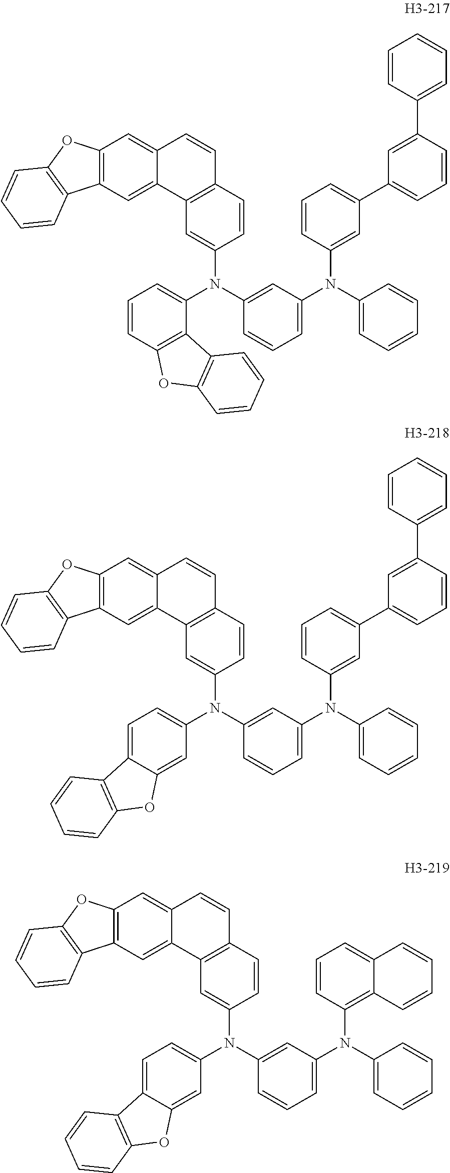 [SMILES: c1ccc(-c2cccc(-c3cccc(N(c4ccccc4)c4cccc(N(c5ccc6ccc7cc8oc9ccccc9c8cc7c6c5)c5cccc6oc7ccccc7c56)c4)c3)c2)cc1 ; c1ccc(-c2cccc(-c3cccc(N(c4ccccc4)c4cccc(N(c5ccc6c(c5)oc5ccccc56)c5ccc6ccc7cc8oc9ccccc9c8cc7c6c5)c4)c3)c2)cc1 ; c1ccc(N(c2cccc(N(c3ccc4c(c3)oc3ccccc34)c3ccc4ccc5cc6oc7ccccc7c6cc5c4c3)c2)c2cccc3ccccc23)cc1]

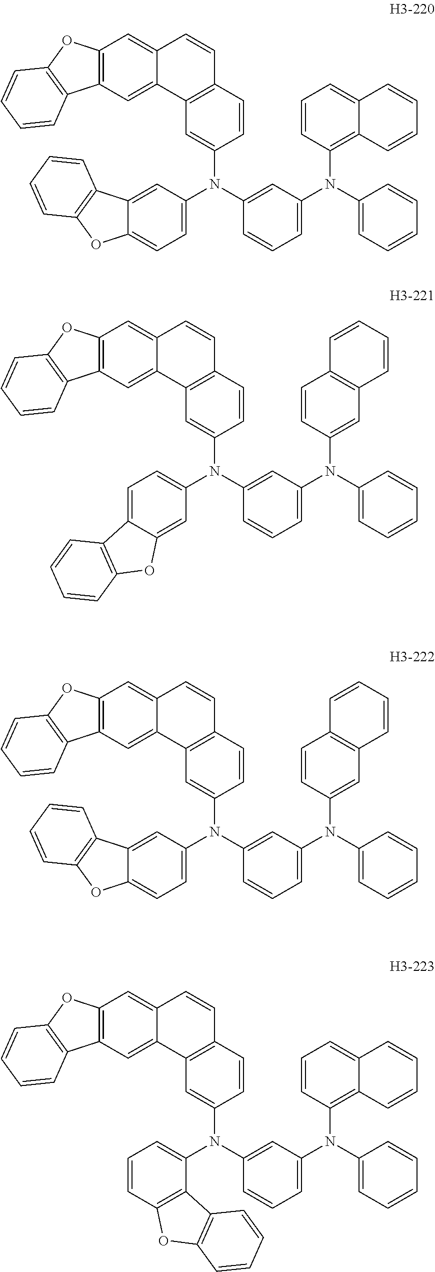 [SMILES: c1ccc(N(c2cccc(N(c3ccc4ccc5cc6oc7ccccc7c6cc5c4c3)c3ccc4oc5ccccc5c4c3)c2)c2cccc3ccccc23)cc1 ; c1ccc(N(c2cccc(N(c3ccc4c(c3)oc3ccccc34)c3ccc4ccc5cc6oc7ccccc7c6cc5c4c3)c2)c2ccc3ccccc3c2)cc1 ; c1ccc(N(c2cccc(N(c3ccc4ccc5cc6oc7ccccc7c6cc5c4c3)c3ccc4oc5ccccc5c4c3)c2)c2ccc3ccccc3c2)cc1 ; c1ccc(N(c2cccc(N(c3ccc4ccc5cc6oc7ccccc7c6cc5c4c3)c3cccc4oc5ccccc5c34)c2)c2cccc3ccccc23)cc1]

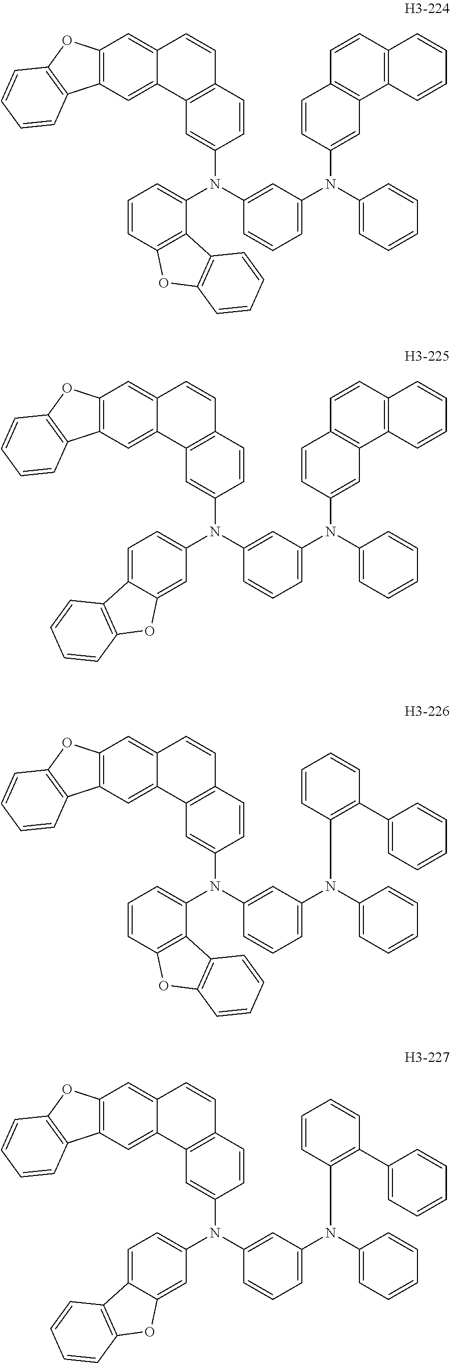 [SMILES: c1ccc(N(c2cccc(N(c3ccc4ccc5cc6oc7ccccc7c6cc5c4c3)c3cccc4oc5ccccc5c34)c2)c2ccc3ccc4ccccc4c3c2)cc1 ; c1ccc(N(c2cccc(N(c3ccc4c(c3)oc3ccccc34)c3ccc4ccc5cc6oc7ccccc7c6cc5c4c3)c2)c2ccc3ccc4ccccc4c3c2)cc1 ; c1ccc(-c2ccccc2CN(c2ccccc2)c2cccc(N(c3ccc4ccc5cc6oc7ccccc7c6cc5c4c3)c3cccc4oc5ccccc5c34)c2)cc1 ; c1ccc(-c2ccccc2CN(c2ccccc2)c2cccc(N(c3ccc4c(c3)oc3ccccc34)c3ccc4ccc5cc6oc7ccccc7c6cc5c4c3)c2)cc1]

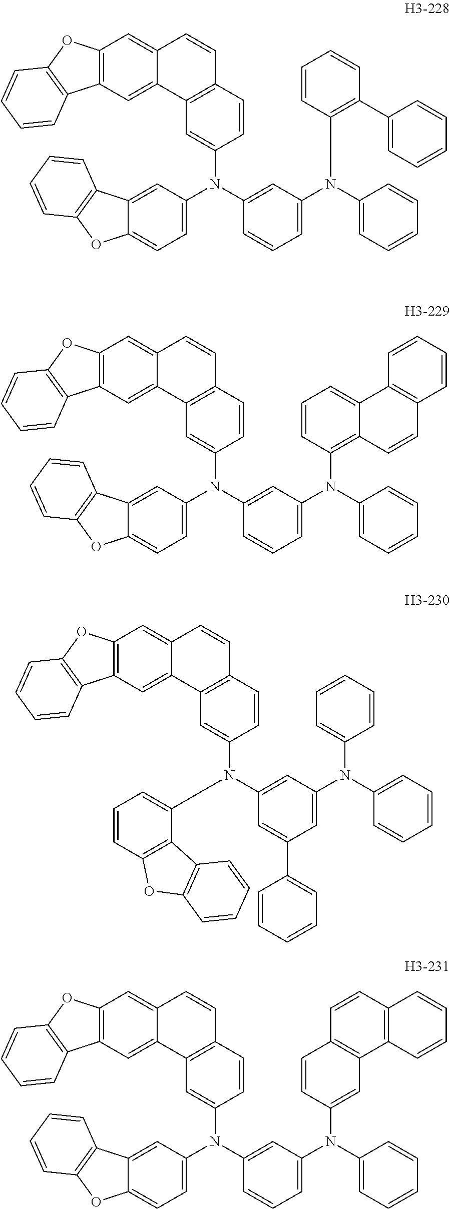 [SMILES: c1ccc(-c2ccccc2CN(c2ccccc2)c2cccc(N(c3ccc4ccc5cc6oc7ccccc7c6cc5c4c3)c3ccc4oc5ccccc5c4c3)c2)cc1 ; c1ccc(N(c2cccc(N(c3ccc4ccc5cc6oc7ccccc7c6cc5c4c3)c3ccc4oc5ccccc5c4c3)c2)c2cccc3c2ccc2ccccc23)cc1 ; c1ccc(-c2cc(N(Cc3cccc4oc5ccccc5c34)c3ccc4ccc5cc6oc7ccccc7c6cc5c4c3)cc(N(c3ccccc3)c3ccccc3)c2)cc1 ; c1ccc(N(c2cccc(N(c3ccc4ccc5cc6oc7ccccc7c6cc5c4c3)c3ccc4oc5ccccc5c4c3)c2)c2ccc3ccc4ccccc4c3c2)cc1]

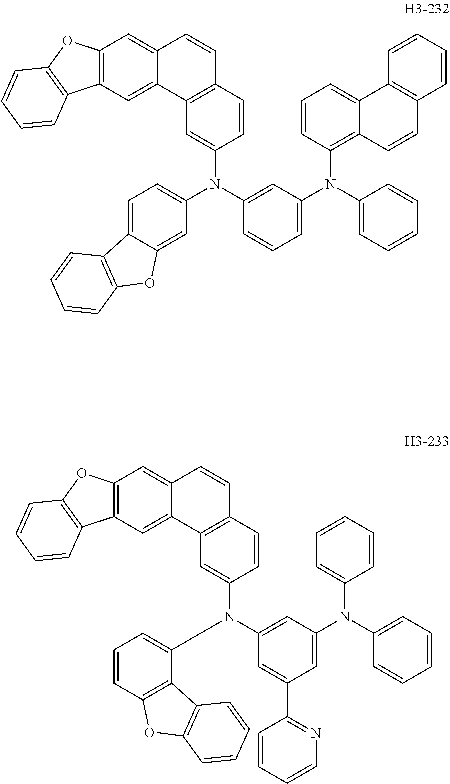 [SMILES: c1ccc(N(c2cccc(N(c3ccc4c(c3)oc3ccccc34)c3ccc4ccc5cc6oc7ccccc7c6cc5c4c3)c2)c2cccc3c2ccc2ccccc23)cc1 ; c1ccc(N(c2ccccc2)c2cc(-c3ccccn3)cc(N(Cc3cccc4oc5ccccc5c34)c3ccc4ccc5cc6oc7ccccc7c6cc5c4c3)c2)cc1]

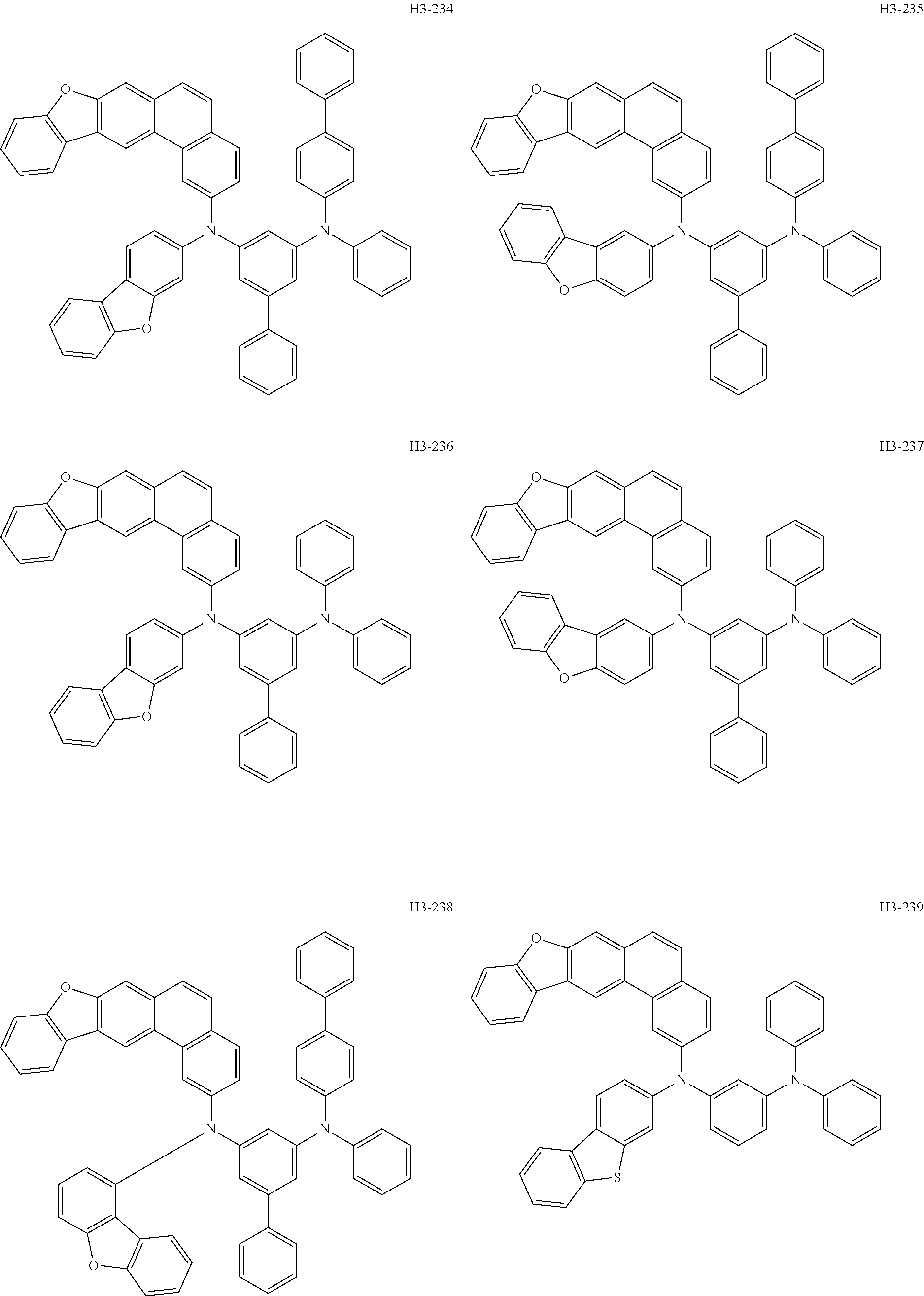

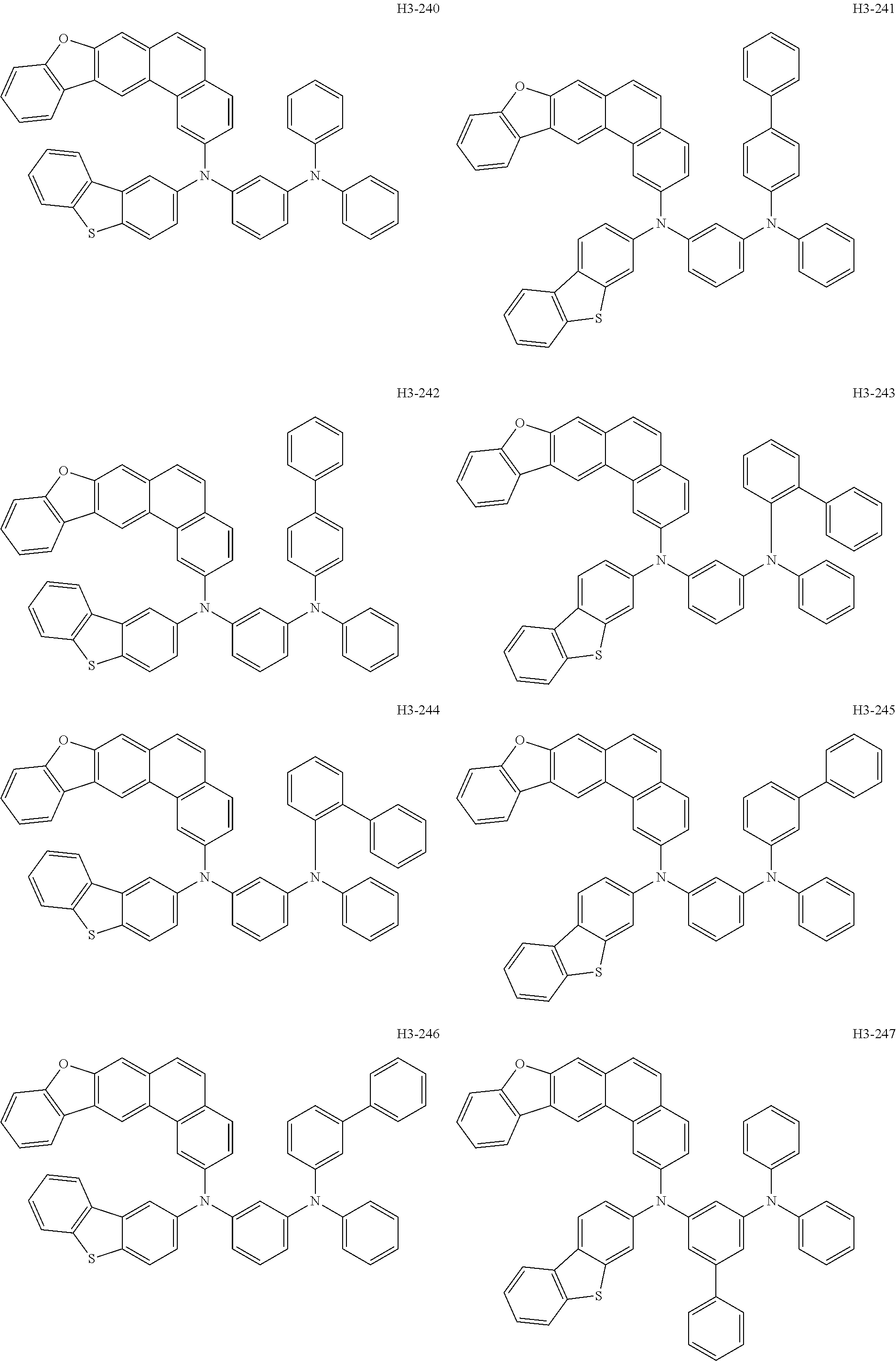

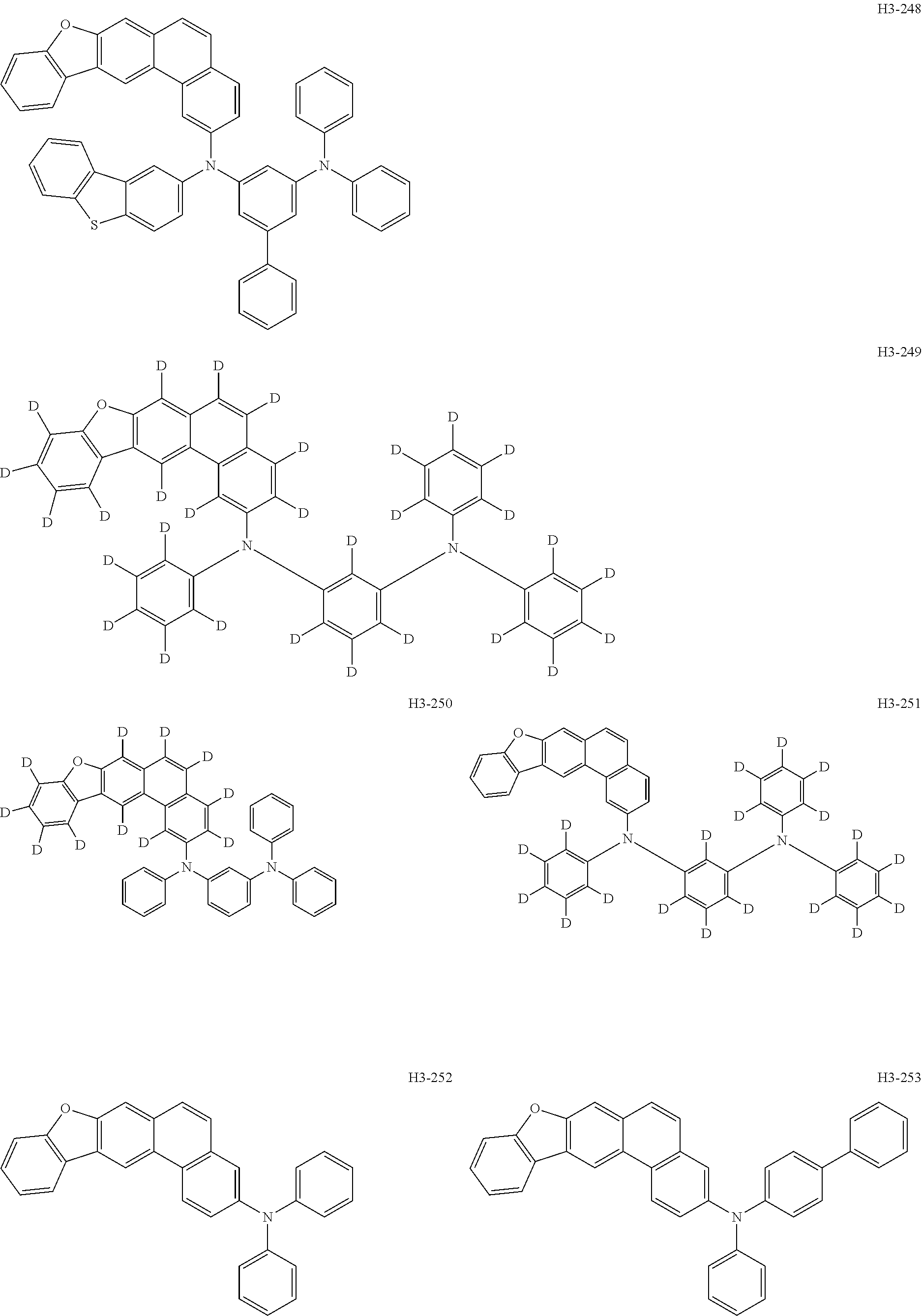

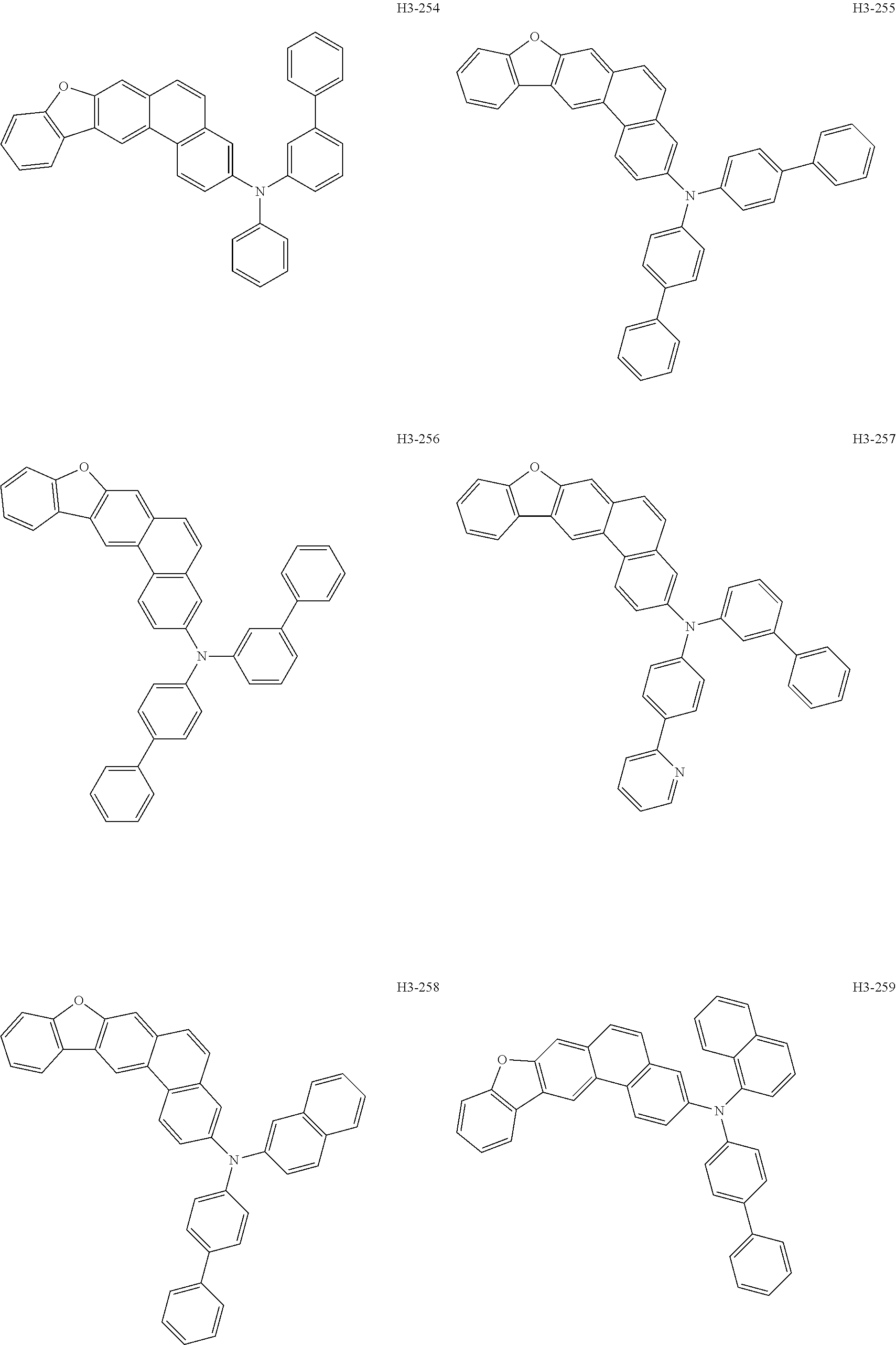 [SMILES: c1ccc(-c2cccc(N(c3ccccc3)c3ccc4c(ccc5cc6oc7ccccc7c6cc54)c3)c2)cc1 ; c1ccc(-c2ccc(N(c3ccc(-c4ccccc4)cc3)c3ccc4c(ccc5cc6oc7ccccc7c6cc54)c3)cc2)cc1 ; c1ccc(-c2ccc(N(c3cccc(-c4ccccc4)c3)c3ccc4c(ccc5cc6oc7ccccc7c6cc54)c3)cc2)cc1 ; c1ccc(-c2cccc(N(c3ccc(-c4ccccn4)cc3)c3ccc4c(ccc5cc6oc7ccccc7c6cc54)c3)c2)cc1 ; c1ccc(-c2ccc(N(c3ccc4ccccc4c3)c3ccc4c(ccc5cc6oc7ccccc7c6cc54)c3)cc2)cc1 ; c1ccc(-c2ccc(N(c3ccc4c(ccc5cc6oc7ccccc7c6cc54)c3)c3cccc4ccccc34)cc2)cc1]

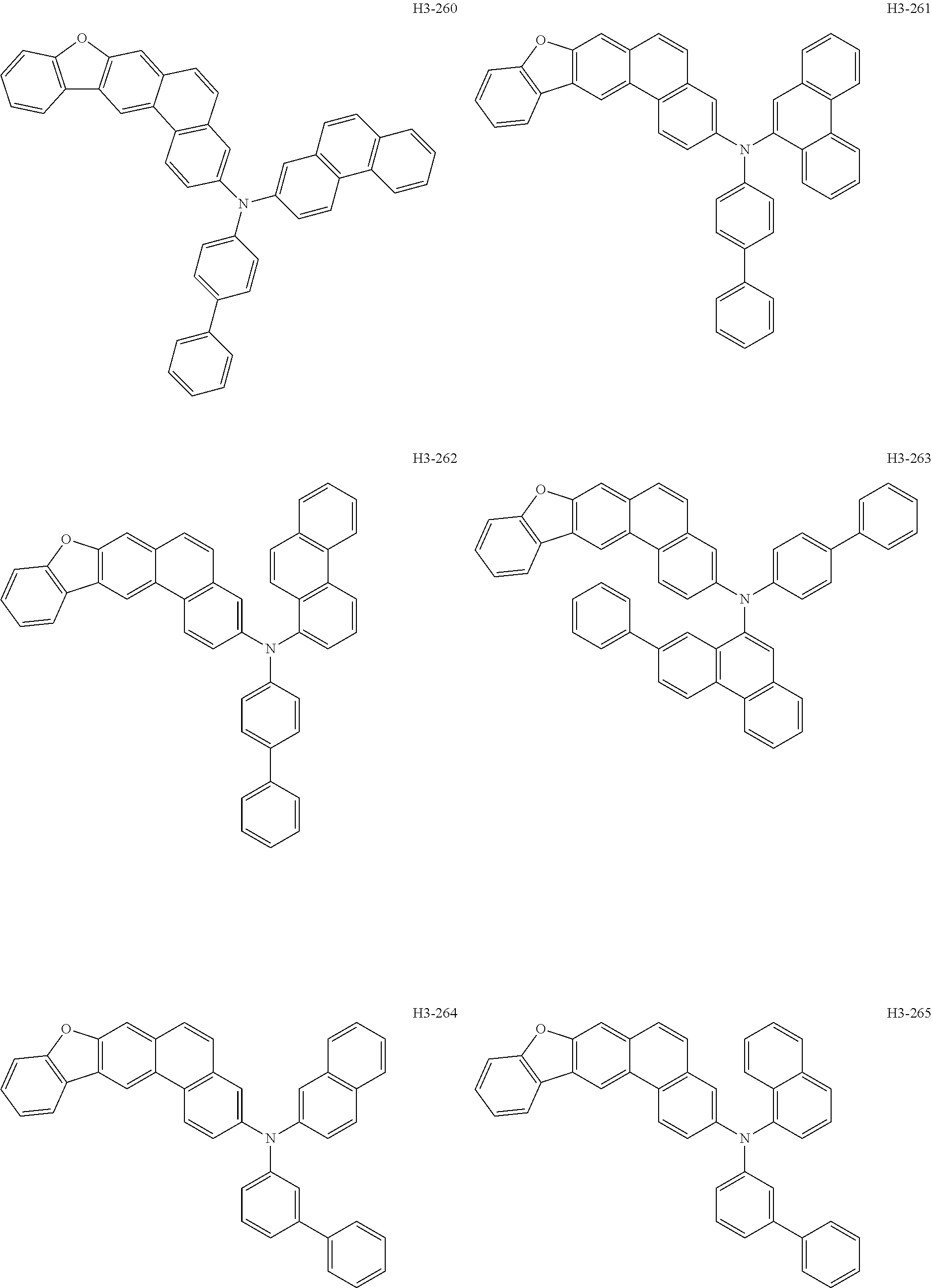 [SMILES: c1ccc(-c2ccc(N(c3ccc4c(ccc5ccccc54)c3)c3ccc4c(ccc5cc6oc7ccccc7c6cc54)c3)cc2)cc1 ; c1ccc(-c2ccc(N(c3ccc4c(ccc5cc6oc7ccccc7c6cc54)c3)c3cc4ccccc4c4ccccc34)cc2)cc1 ; c1ccc(-c2ccc(N(c3ccc4c(ccc5cc6oc7ccccc7c6cc54)c3)c3cccc4c3ccc3ccccc34)cc2)cc1 ; c1ccc(-c2ccc(N(c3ccc4c(ccc5cc6oc7ccccc7c6cc54)c3)c3cc4ccccc4c4ccc(-c5ccccc5)cc34)cc2)cc1 ; c1ccc(-c2cccc(N(c3ccc4ccccc4c3)c3ccc4c(ccc5cc6oc7ccccc7c6cc54)c3)c2)cc1 ; c1ccc(-c2cccc(N(c3ccc4c(ccc5cc6oc7ccccc7c6cc54)c3)c3cccc4ccccc34)c2)cc1]

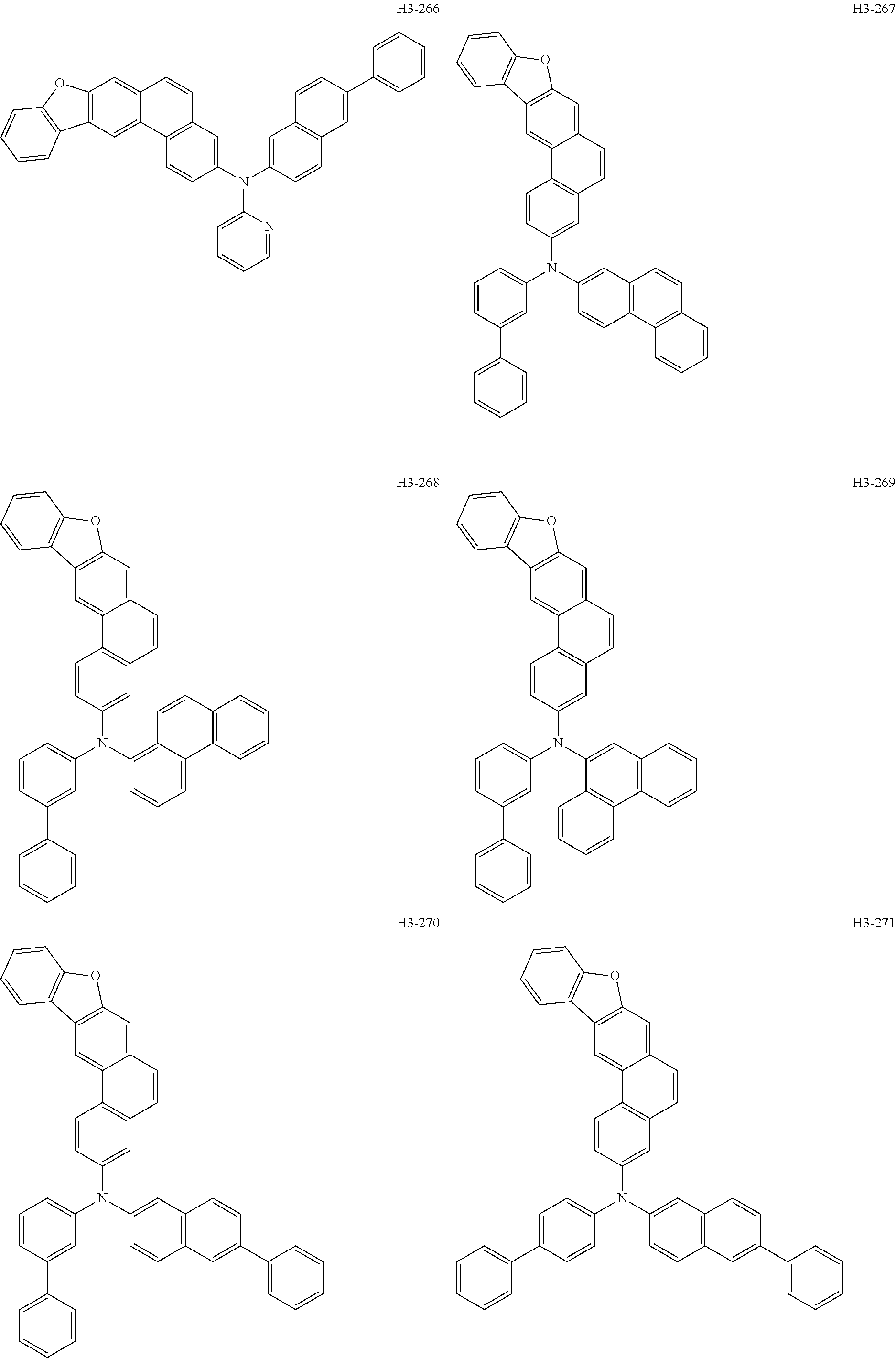

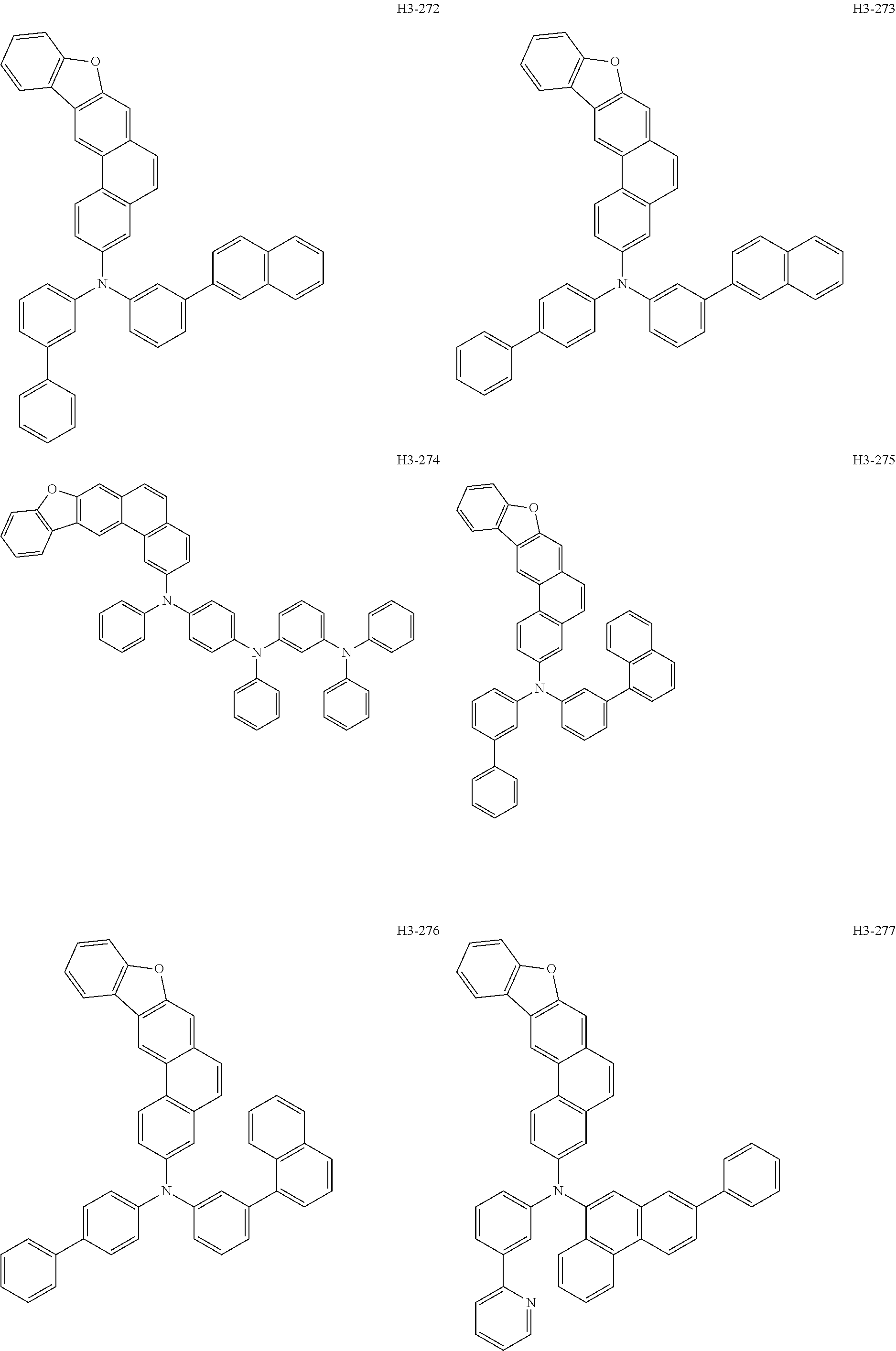

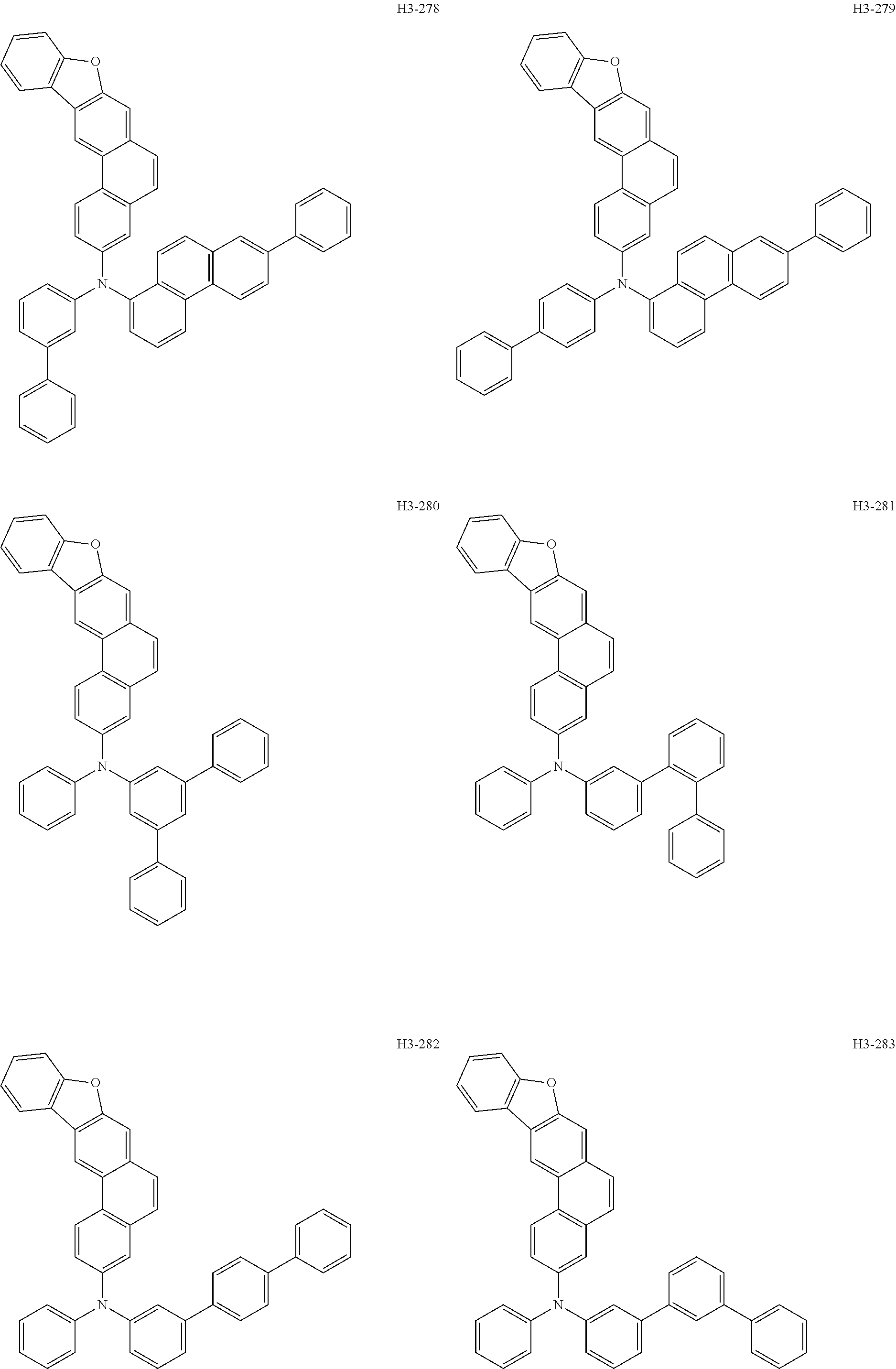 [SMILES: c1ccc(-c2cccc(N(c3ccc4c(ccc5cc6oc7ccccc7c6cc54)c3)c3cccc4c3ccc3cc(-c5ccccc5)ccc34)c2)cc1 ; c1ccc(-c2ccc(N(c3ccc4c(ccc5cc6oc7ccccc7c6cc54)c3)c3cccc4c3ccc3cc(-c5ccccc5)ccc34)cc2)cc1 ; c1ccc(-c2cc(-c3ccccc3)cc(N(c3ccccc3)c3ccc4c(ccc5cc6oc7ccccc7c6cc54)c3)c2)cc1 ; c1ccc(-c2ccccc2-c2cccc(N(c3ccccc3)c3ccc4c(ccc5cc6oc7ccccc7c6cc54)c3)c2)cc1 ; c1ccc(-c2ccc(-c3cccc(N(c4ccccc4)c4ccc5c(ccc6cc7oc8ccccc8c7cc65)c4)c3)cc2)cc1 ; c1ccc(-c2cccc(-c3cccc(N(c4ccccc4)c4ccc5c(ccc6cc7oc8ccccc8c7cc65)c4)c3)c2)cc1]

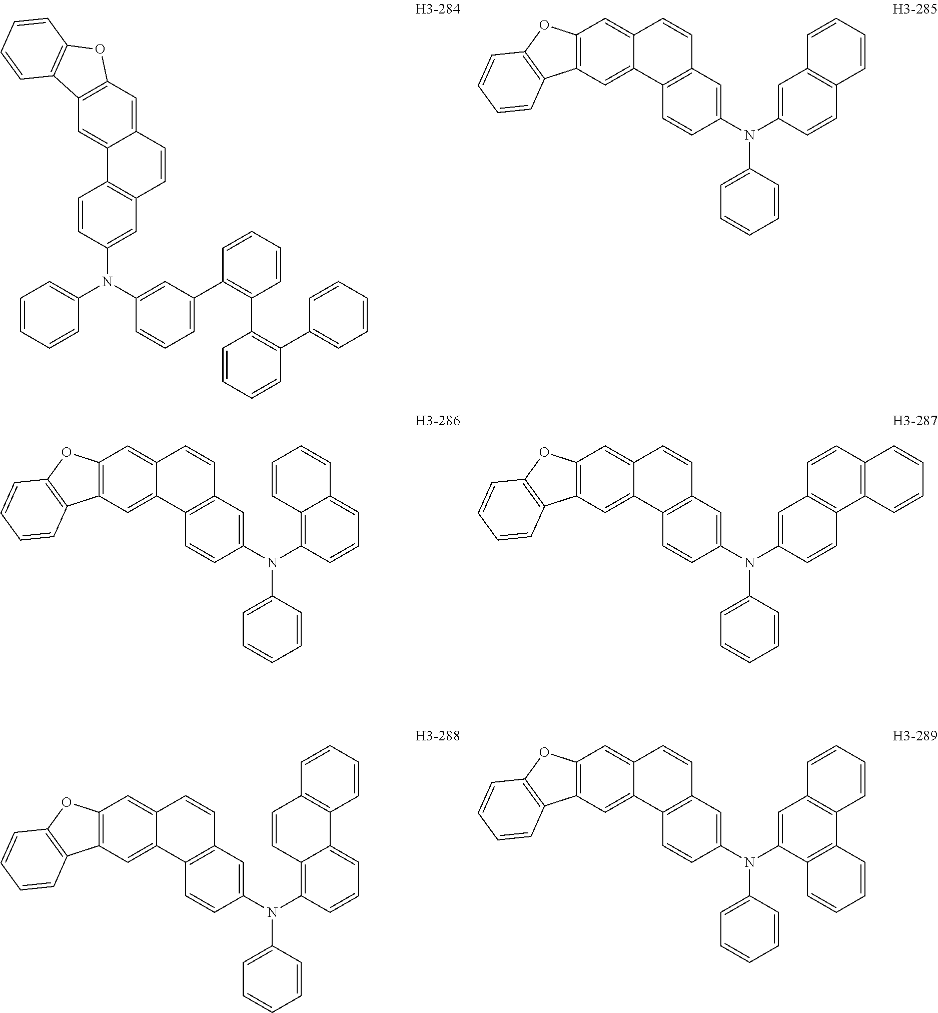 [SMILES: c1ccc(-c2ccccc2-c2ccccc2-c2cccc(N(c3ccccc3)c3ccc4c(ccc5cc6oc7ccccc7c6cc54)c3)c2)cc1 ; c1ccc(N(c2ccc3ccccc3c2)c2ccc3c(ccc4cc5oc6ccccc6c5cc43)c2)cc1 ; c1ccc(N(c2ccc3c(ccc4cc5oc6ccccc6c5cc43)c2)c2cccc3ccccc23)cc1 ; c1ccc(N(c2ccc3c(ccc4ccccc43)c2)c2ccc3c(ccc4cc5oc6ccccc6c5cc43)c2)cc1 ; c1ccc(N(c2ccc3c(ccc4cc5oc6ccccc6c5cc43)c2)c2cccc3c2ccc2ccccc23)cc1 ; c1ccc(N(c2ccc3c(ccc4cc5oc6ccccc6c5cc43)c2)c2cc3ccccc3c3ccccc23)cc1]

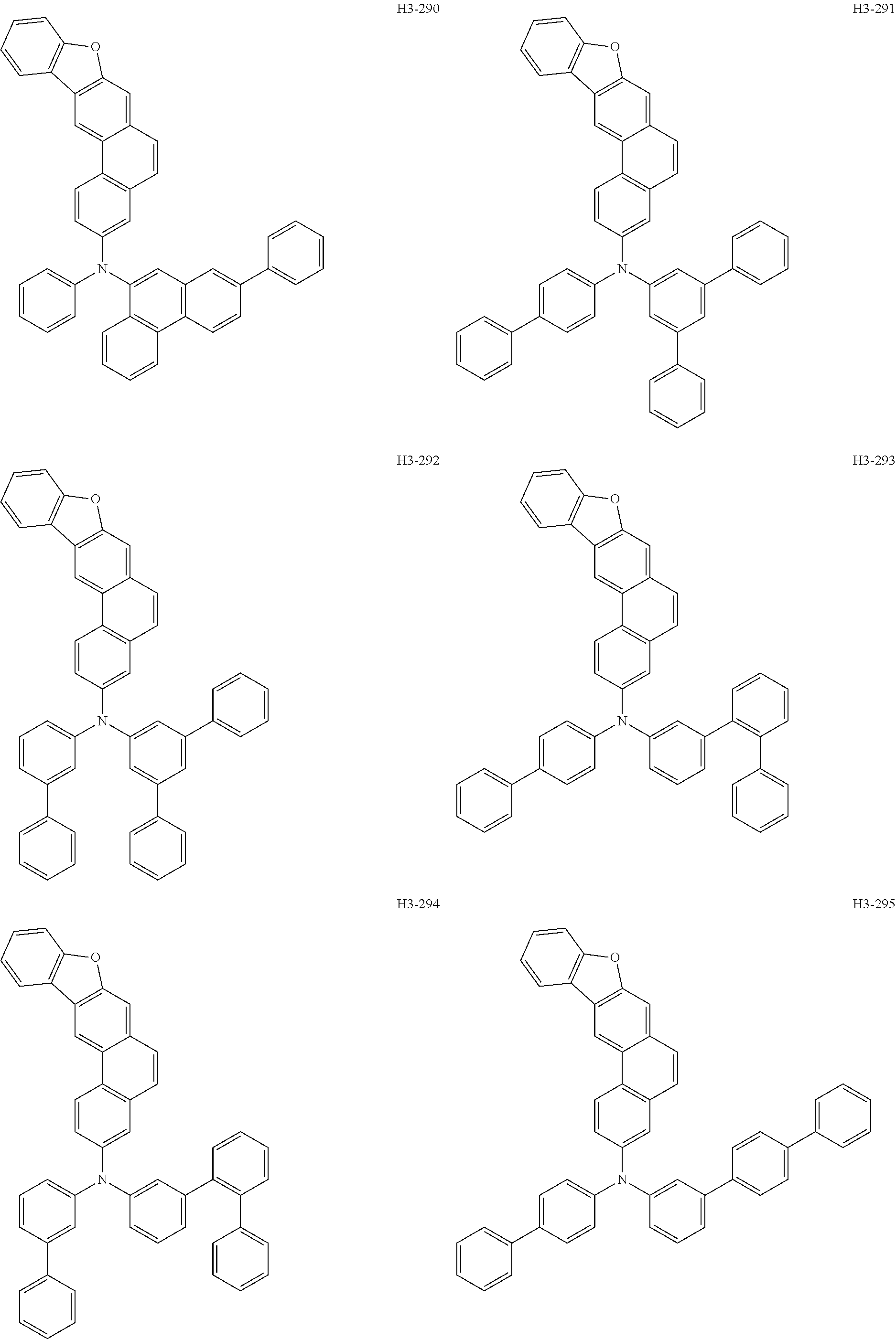

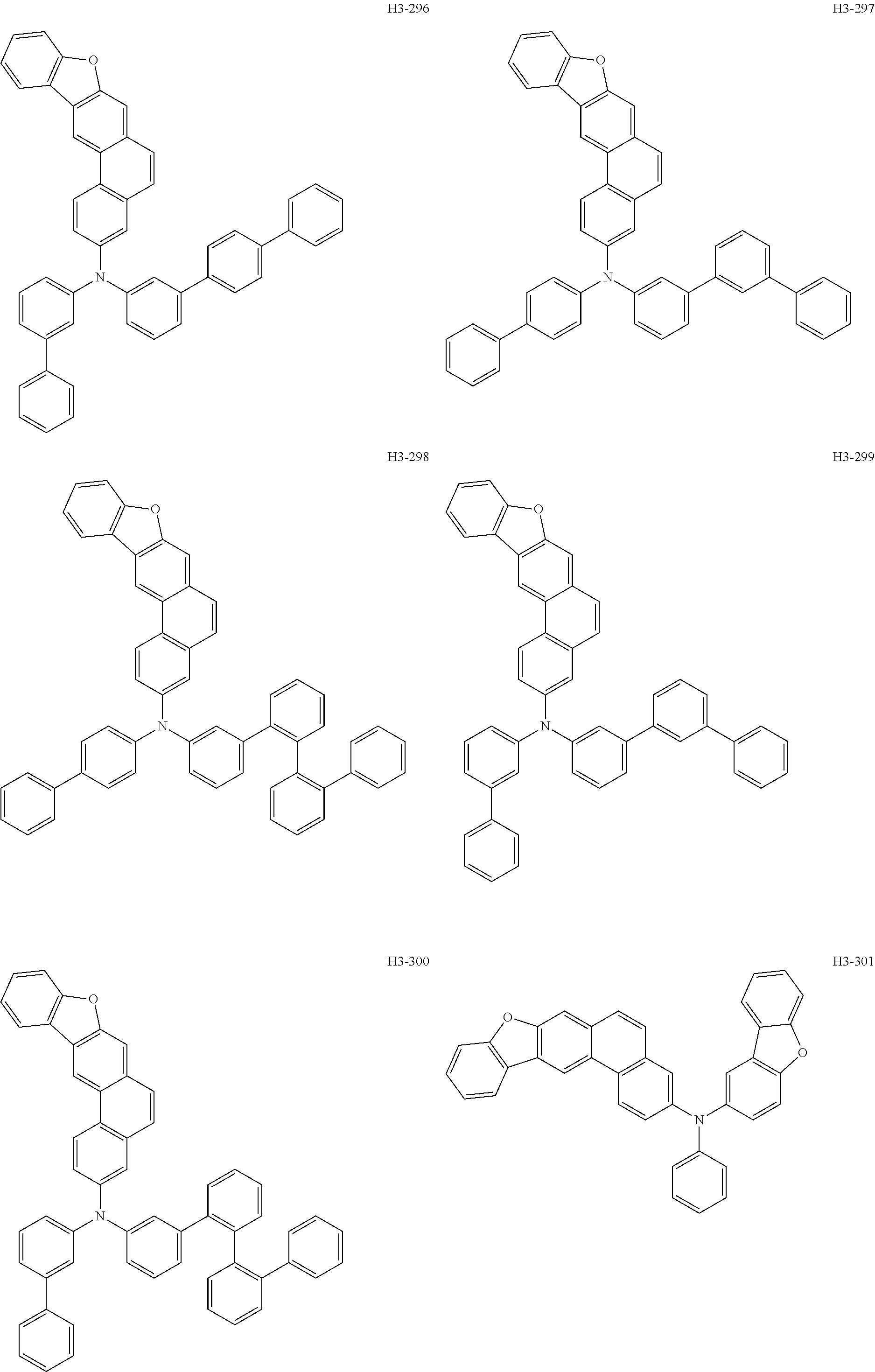

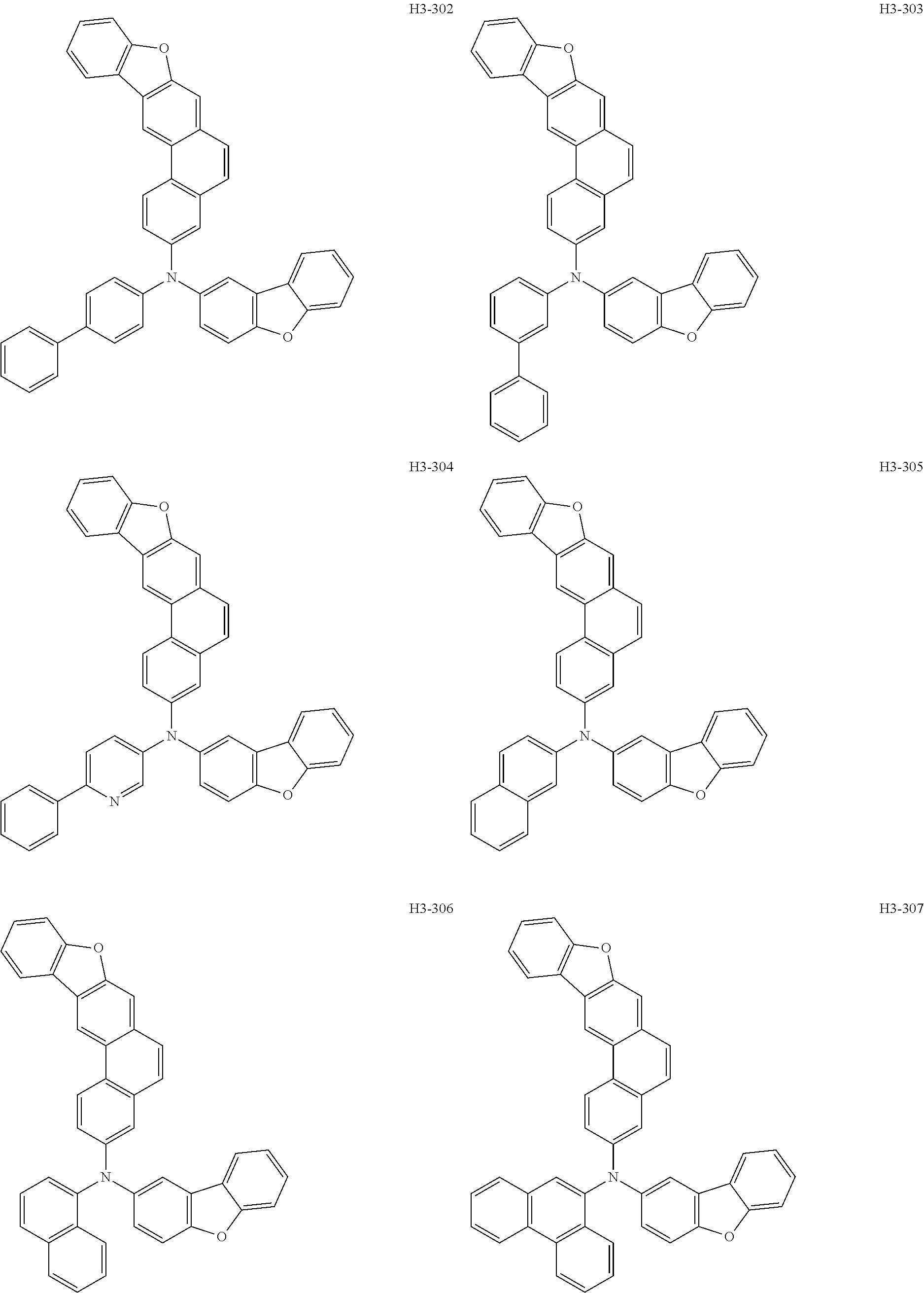

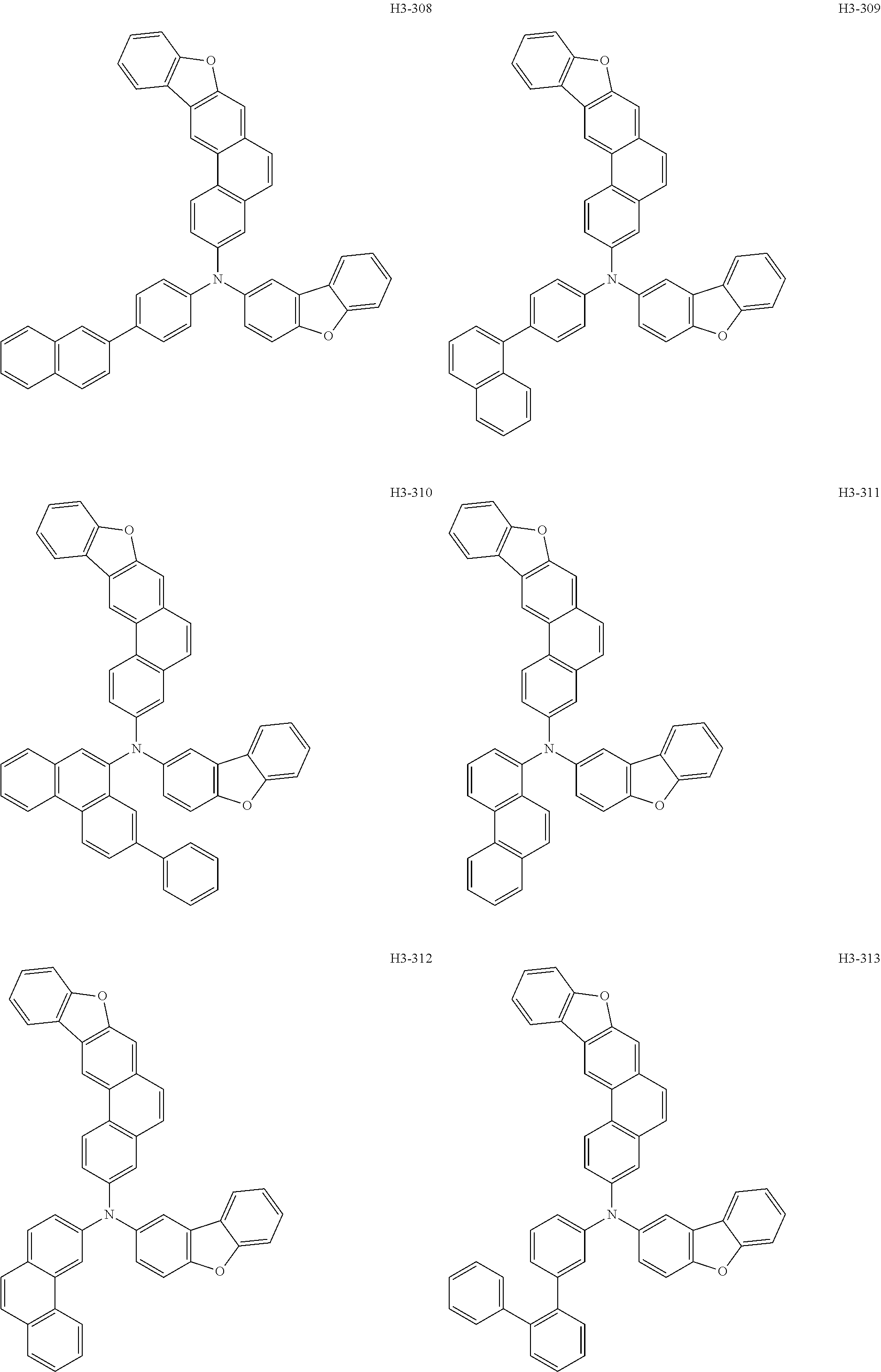

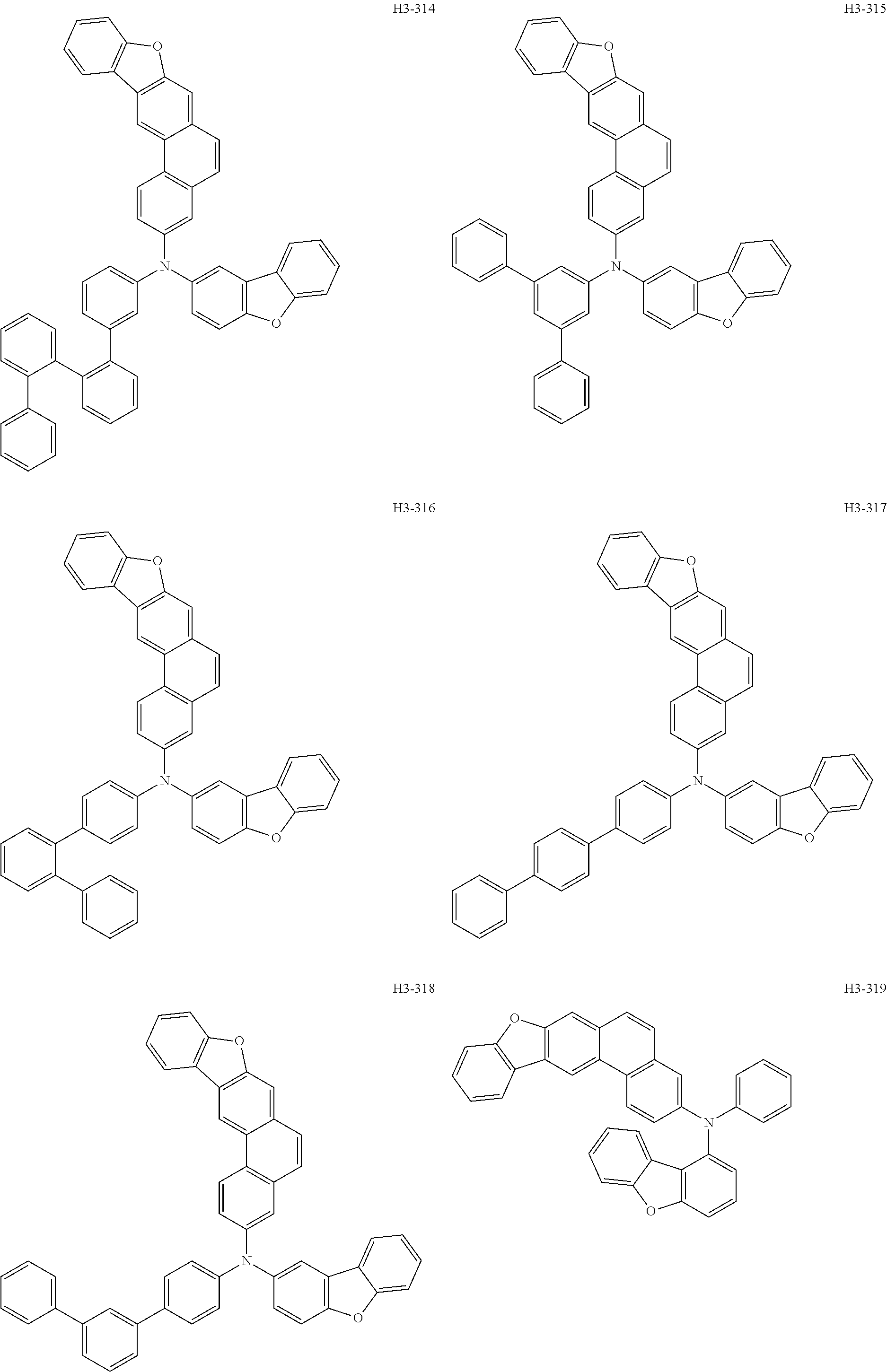

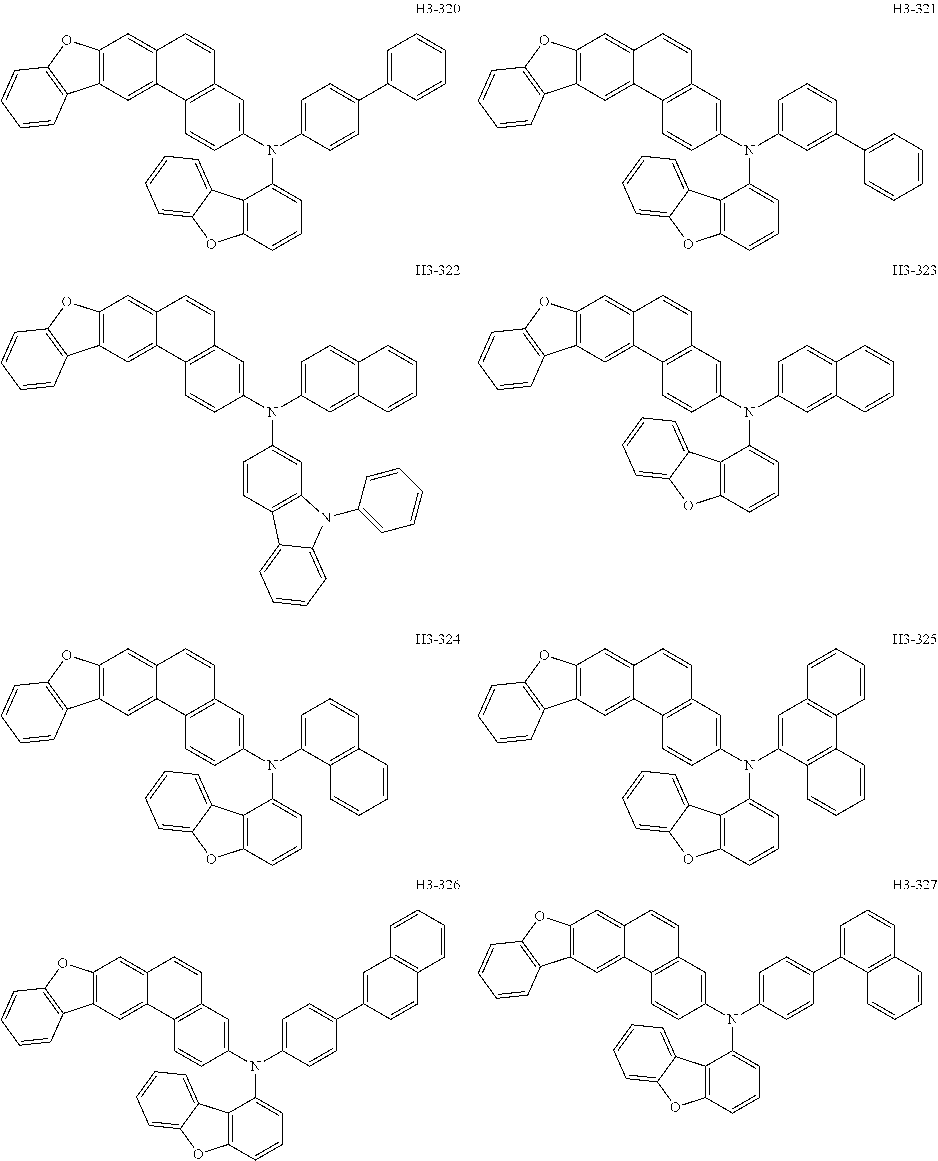

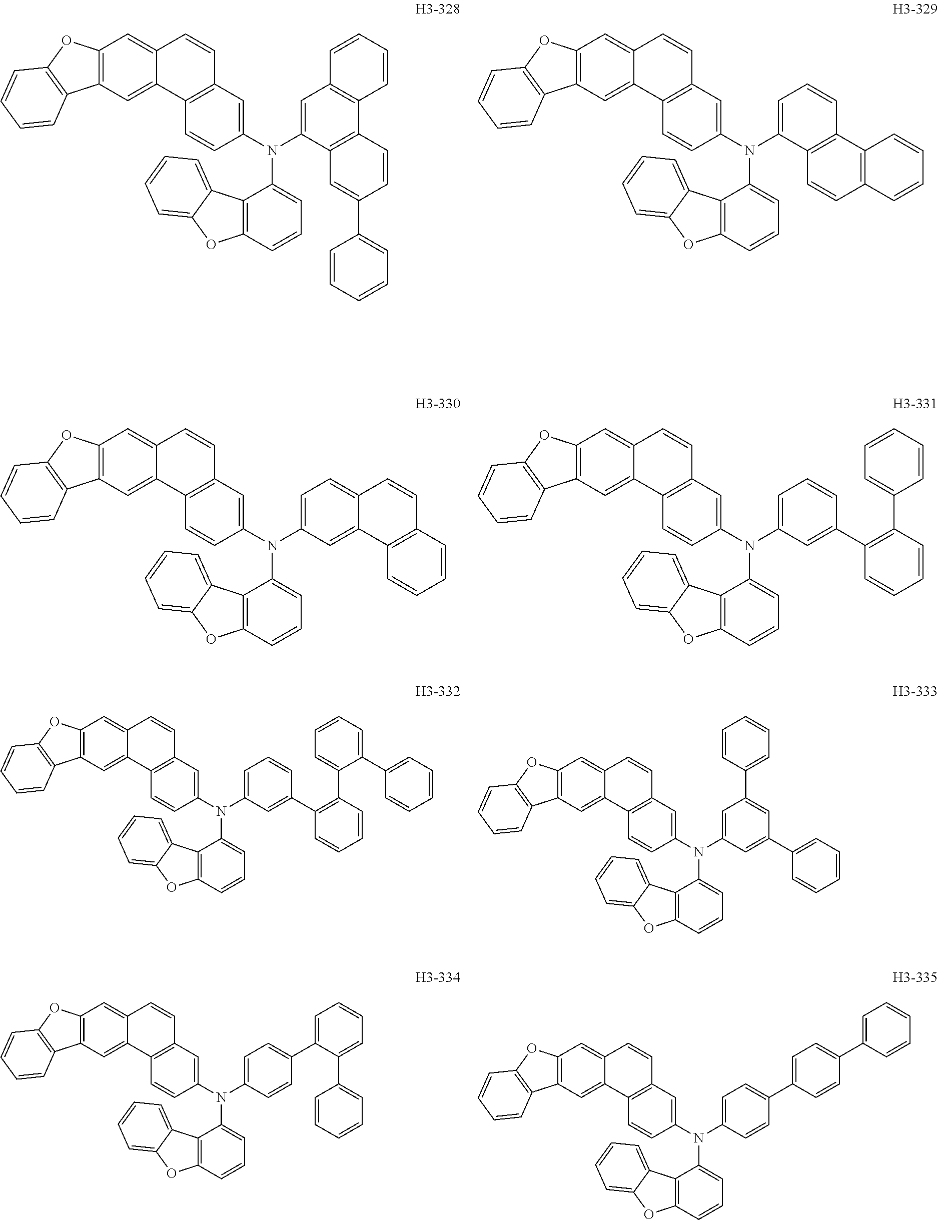

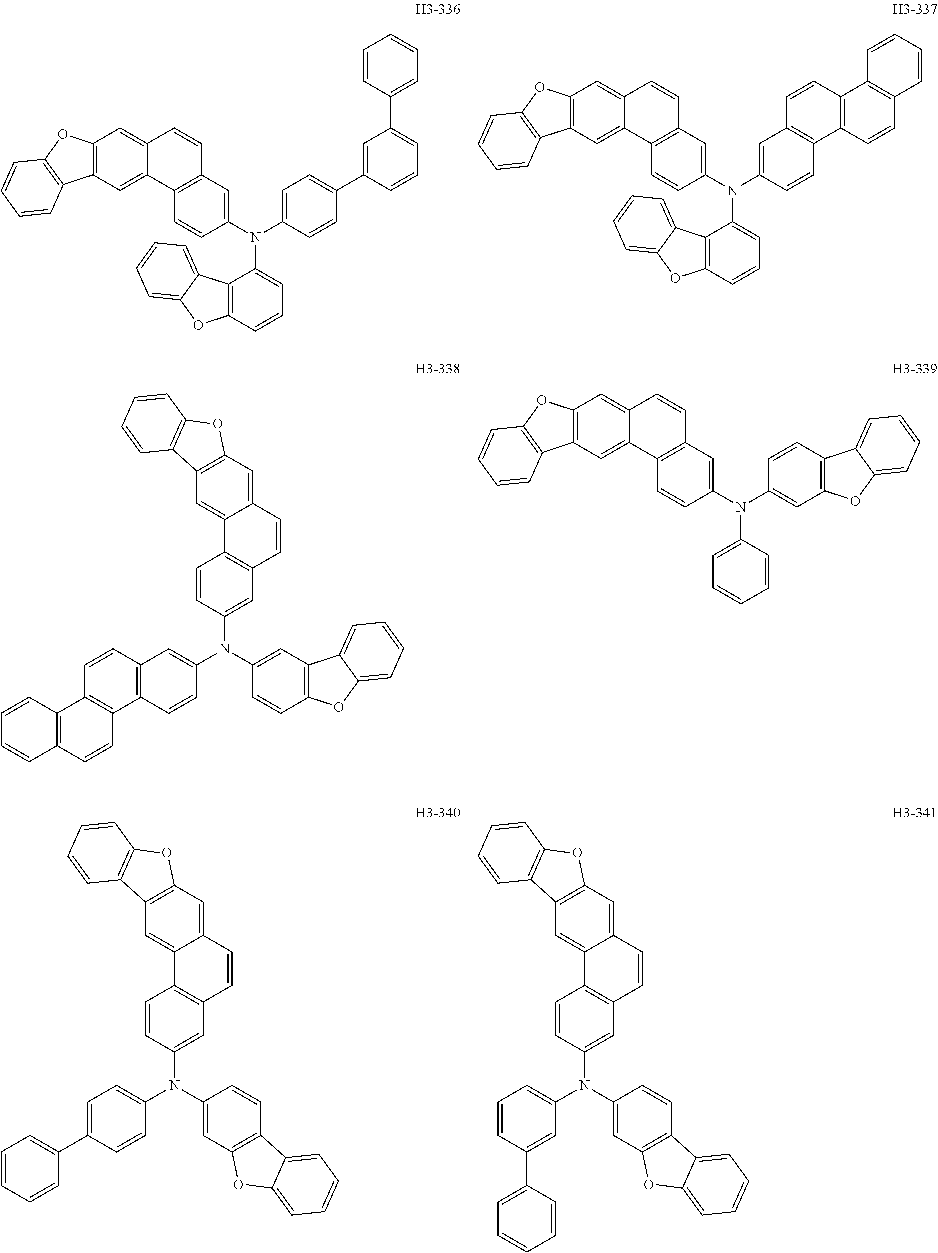 [SMILES: c1ccc(-c2cccc(-c3ccc(N(c4ccc5c(ccc6cc7oc8ccccc8c7cc65)c4)c4cccc5oc6ccccc6c45)cc3)c2)cc1 ; c1ccc2c(c1)ccc1c3ccc(N(c4ccc5c(ccc6cc7oc8ccccc8c7cc65)c4)c4cccc5oc6ccccc6c45)cc3ccc21 ; c1ccc2c(c1)ccc1c3ccc(N(c4ccc5c(ccc6cc7oc8ccccc8c7cc65)c4)c4ccc5oc6ccccc6c5c4)cc3ccc21 ; c1ccc(N(c2ccc3c(ccc4cc5oc6ccccc6c5cc43)c2)c2ccc3c(c2)oc2ccccc23)cc1 ; c1ccc(-c2ccc(N(c3ccc4c(ccc5cc6oc7ccccc7c6cc54)c3)c3ccc4c(c3)oc3ccccc34)cc2)cc1 ; c1ccc(-c2cccc(N(c3ccc4c(ccc5cc6oc7ccccc7c6cc54)c3)c3ccc4c(c3)oc3ccccc34)c2)cc1]

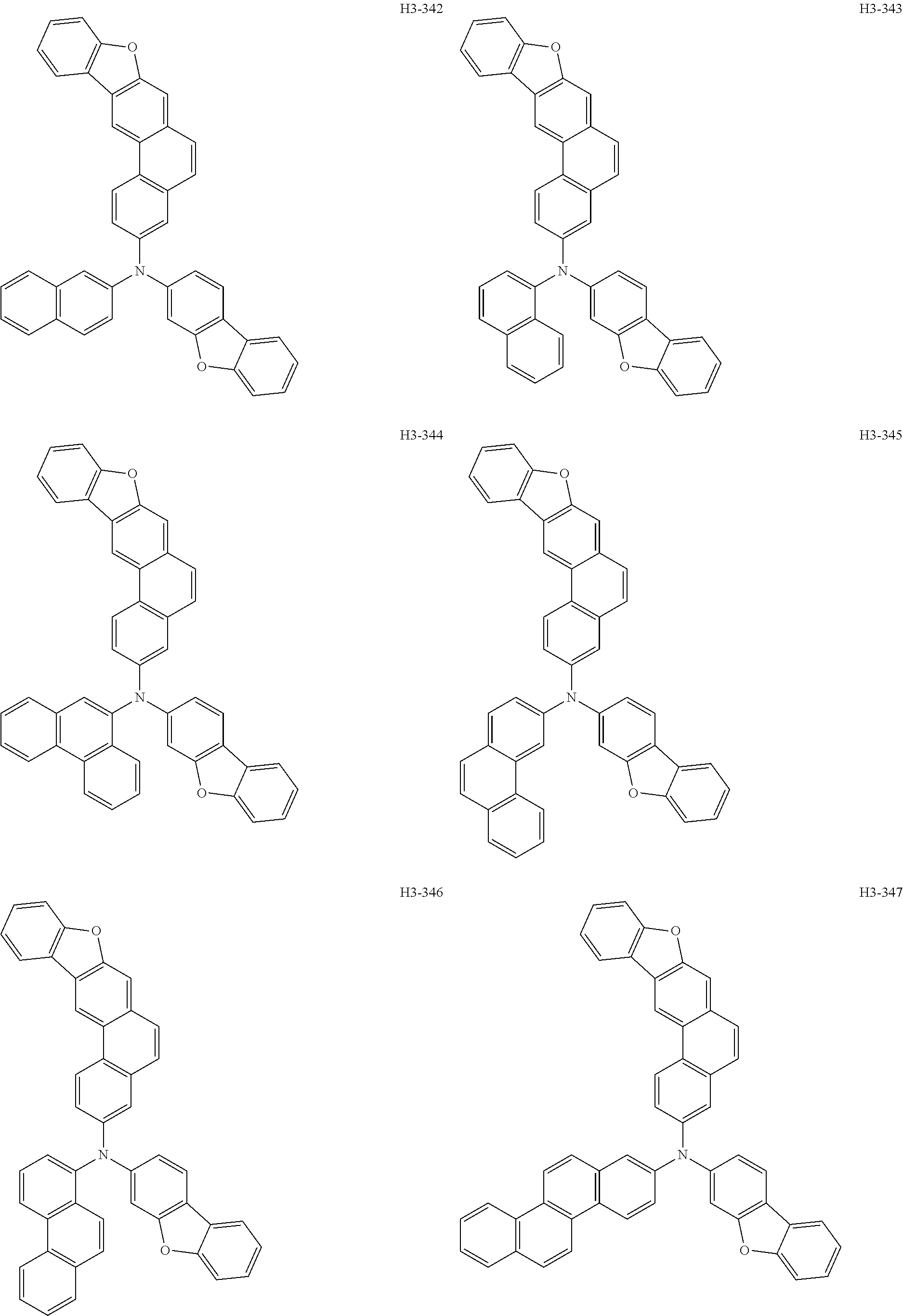 [SMILES: c1ccc2cc(N(c3ccc4c(ccc5cc6oc7ccccc7c6cc54)c3)c3ccc4c(c3)oc3ccccc34)ccc2c1 ; c1ccc2c(N(c3ccc4c(ccc5cc6oc7ccccc7c6cc54)c3)c3ccc4c(c3)oc3ccccc34)cccc2c1 ; c1ccc2c(c1)cc(N(c1ccc3c(ccc4cc5oc6ccccc6c5cc43)c1)c1ccc3c(c1)oc1ccccc13)c1ccccc12 ; c1ccc2c(c1)ccc1ccc(N(c3ccc4c(ccc5cc6oc7ccccc7c6cc54)c3)c3ccc4c(c3)oc3ccccc34)cc12 ; c1ccc2c(c1)ccc1c(N(c3ccc4c(ccc5cc6oc7ccccc7c6cc54)c3)c3ccc4c(c3)oc3ccccc34)cccc12 ; c1ccc2c(c1)ccc1c3ccc(N(c4ccc5c(ccc6cc7oc8ccccc8c7cc65)c4)c4ccc5c(c4)oc4ccccc45)cc3ccc21]

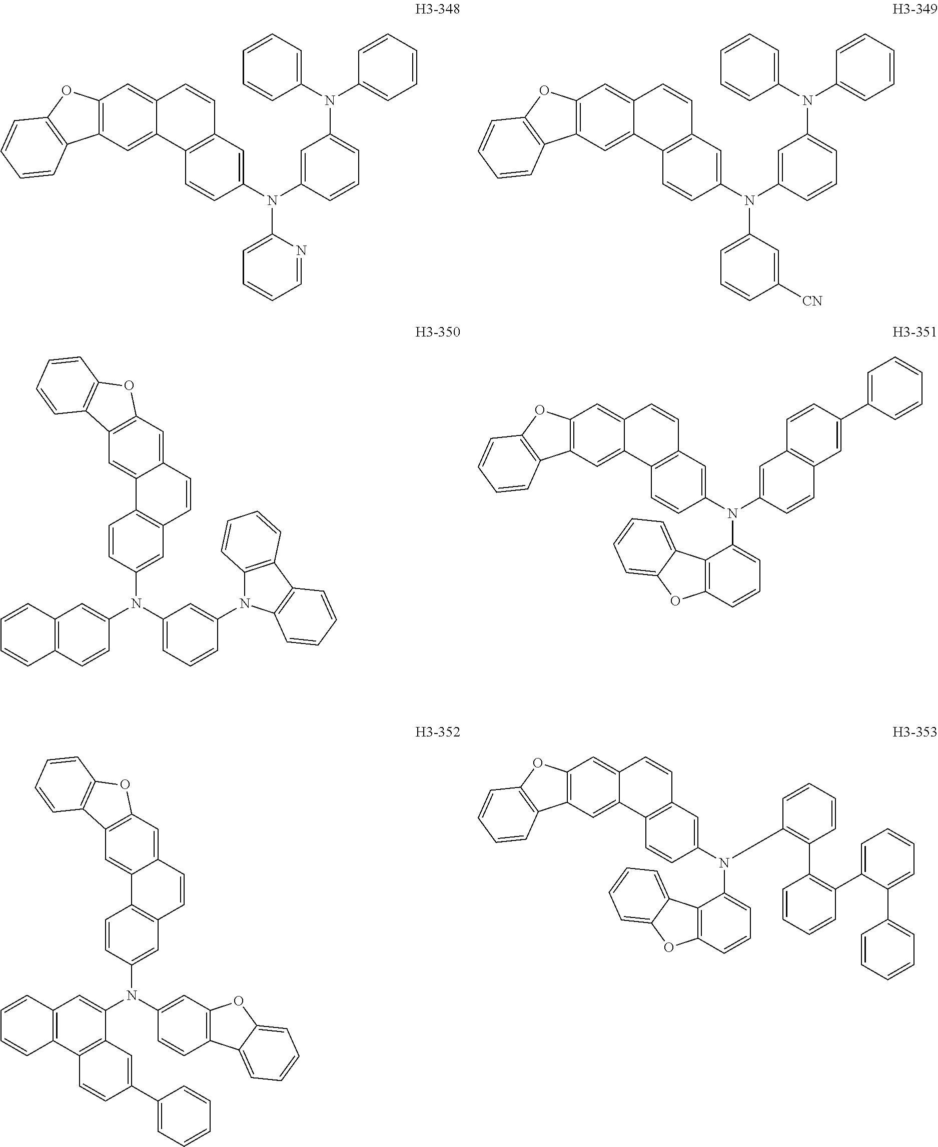

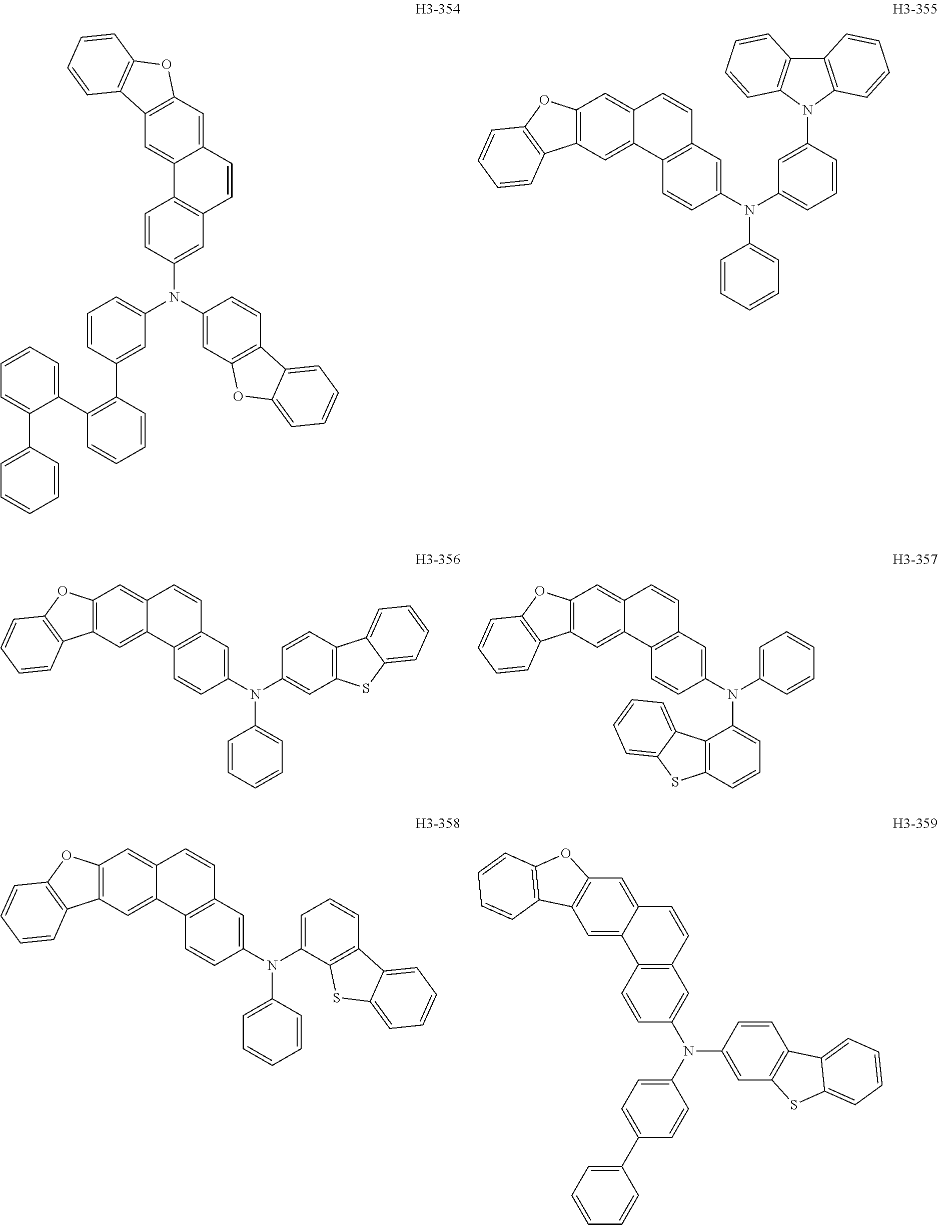 [SMILES: c1ccc(-c2ccccc2-c2ccccc2-c2cccc(N(c3ccc4c(ccc5cc6oc7ccccc7c6cc54)c3)c3ccc4c(c3)oc3ccccc34)c2)cc1 ; c1ccc(N(c2cccc(-n3c4ccccc4c4ccccc43)c2)c2ccc3c(ccc4cc5oc6ccccc6c5cc43)c2)cc1 ; c1ccc(N(c2ccc3c(ccc4cc5oc6ccccc6c5cc43)c2)c2ccc3c(c2)sc2ccccc23)cc1 ; c1ccc(N(c2ccc3c(ccc4cc5oc6ccccc6c5cc43)c2)c2cccc3sc4ccccc4c23)cc1 ; c1ccc(N(c2ccc3c(ccc4cc5oc6ccccc6c5cc43)c2)c2cccc3c2sc2ccccc23)cc1 ; c1ccc(-c2ccc(N(c3ccc4c(ccc5cc6oc7ccccc7c6cc54)c3)c3ccc4c(c3)sc3ccccc34)cc2)cc1]

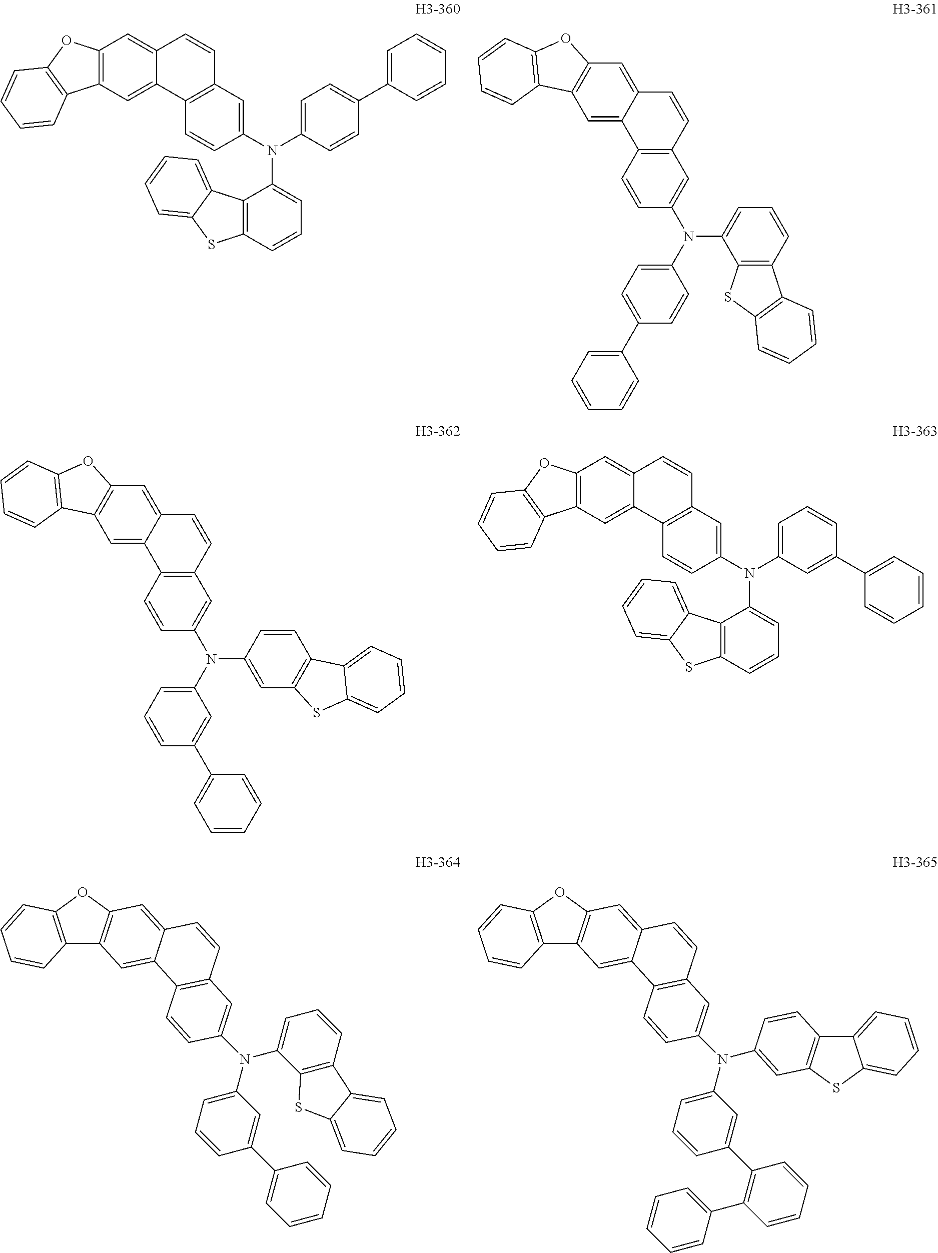

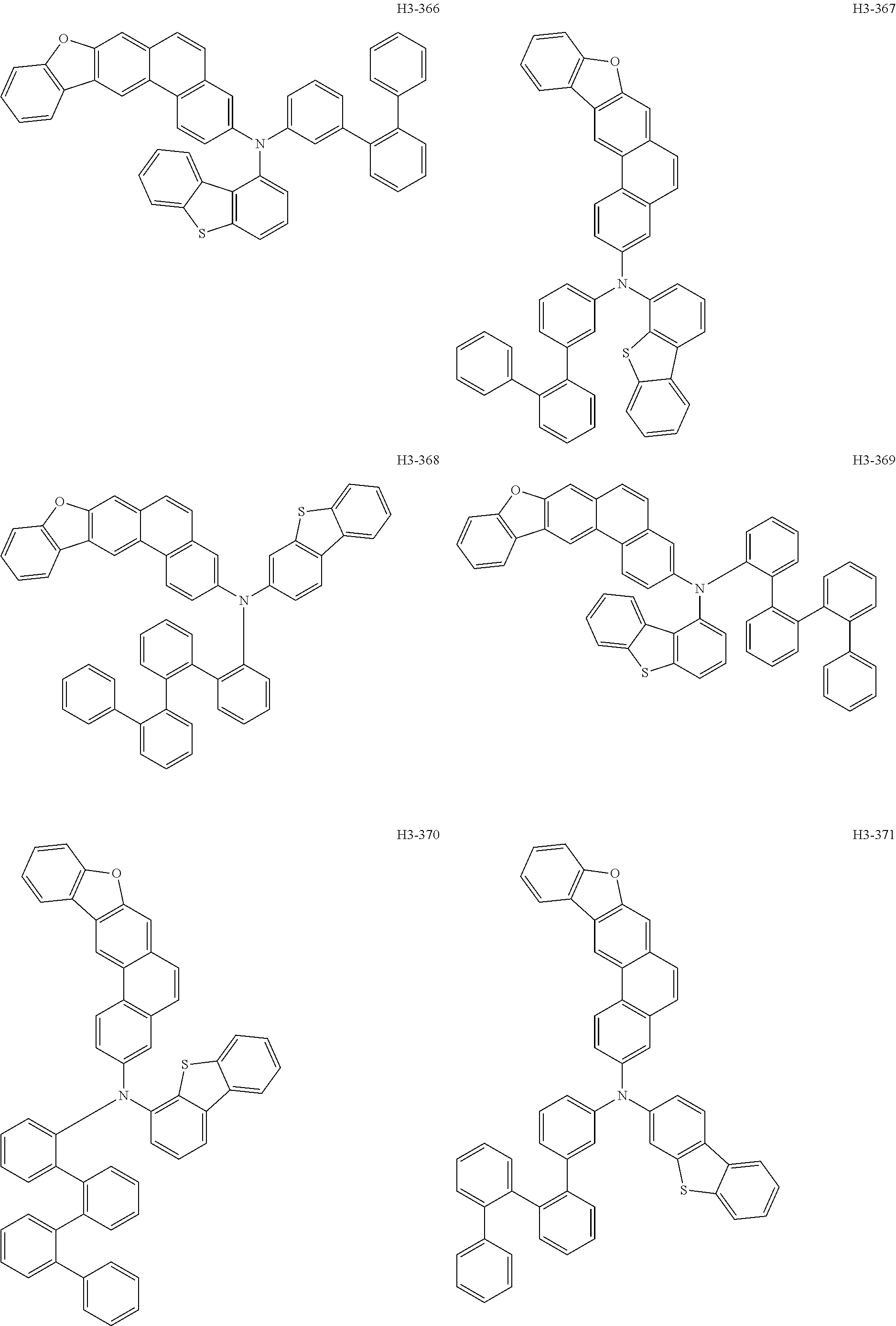

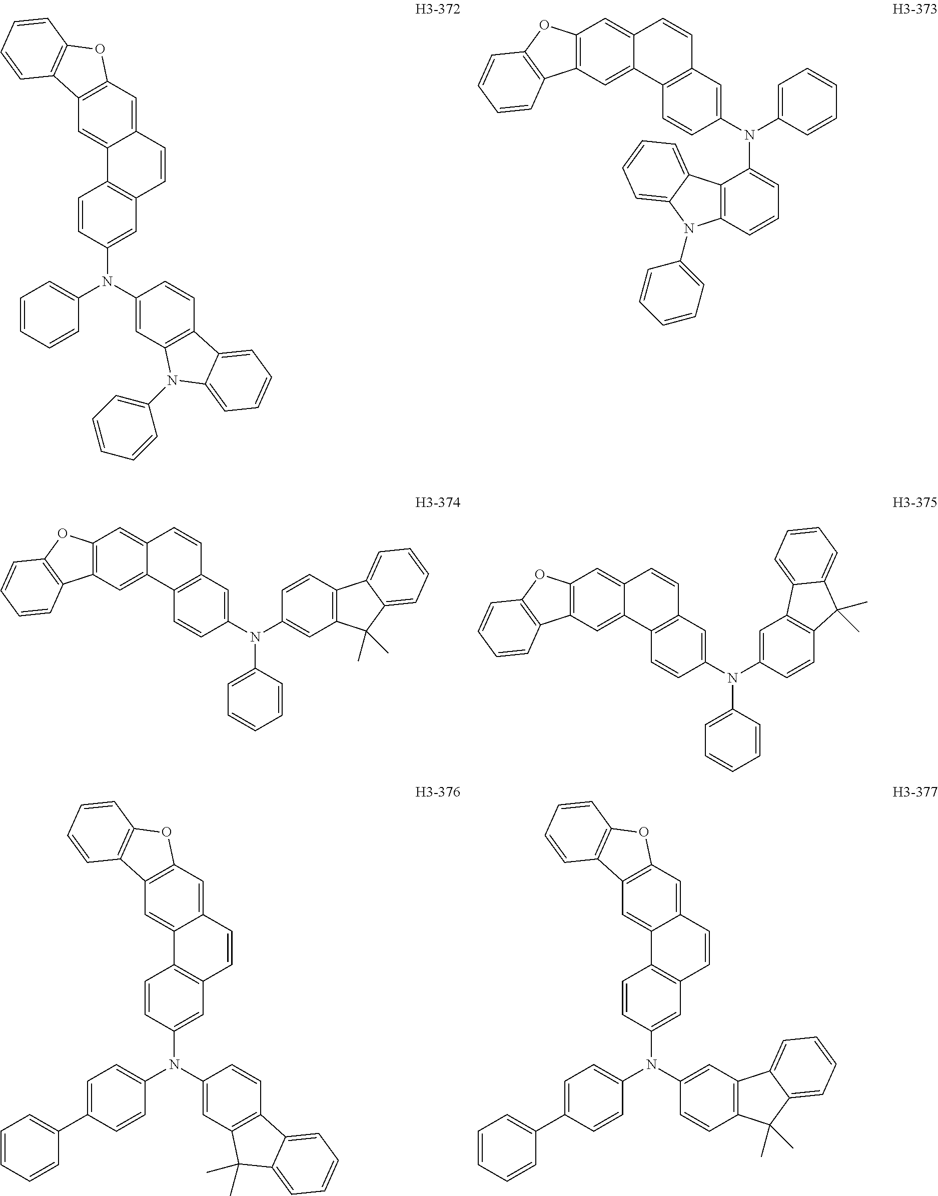 [SMILES: c1ccc(N(c2ccc3c(ccc4cc5oc6ccccc6c5cc43)c2)c2ccc3c4ccccc4n(-c4ccccc4)c3c2)cc1 ; c1ccc(N(c2ccc3c(ccc4cc5oc6ccccc6c5cc43)c2)c2cccc3c2c2ccccc2n3-c2ccccc2)cc1 ; CC1(C)c2ccccc2-c2ccc(N(c3ccccc3)c3ccc4c(ccc5cc6oc7ccccc7c6cc54)c3)cc21 ; CC1(C)c2ccccc2-c2cc(N(c3ccccc3)c3ccc4c(ccc5cc6oc7ccccc7c6cc54)c3)ccc21 ; CC1(C)c2ccccc2-c2ccc(N(c3ccc(-c4ccccc4)cc3)c3ccc4c(ccc5cc6oc7ccccc7c6cc54)c3)cc21 ; CC1(C)c2ccccc2-c2cc(N(c3ccc(-c4ccccc4)cc3)c3ccc4c(ccc5cc6oc7ccccc7c6cc54)c3)ccc21]

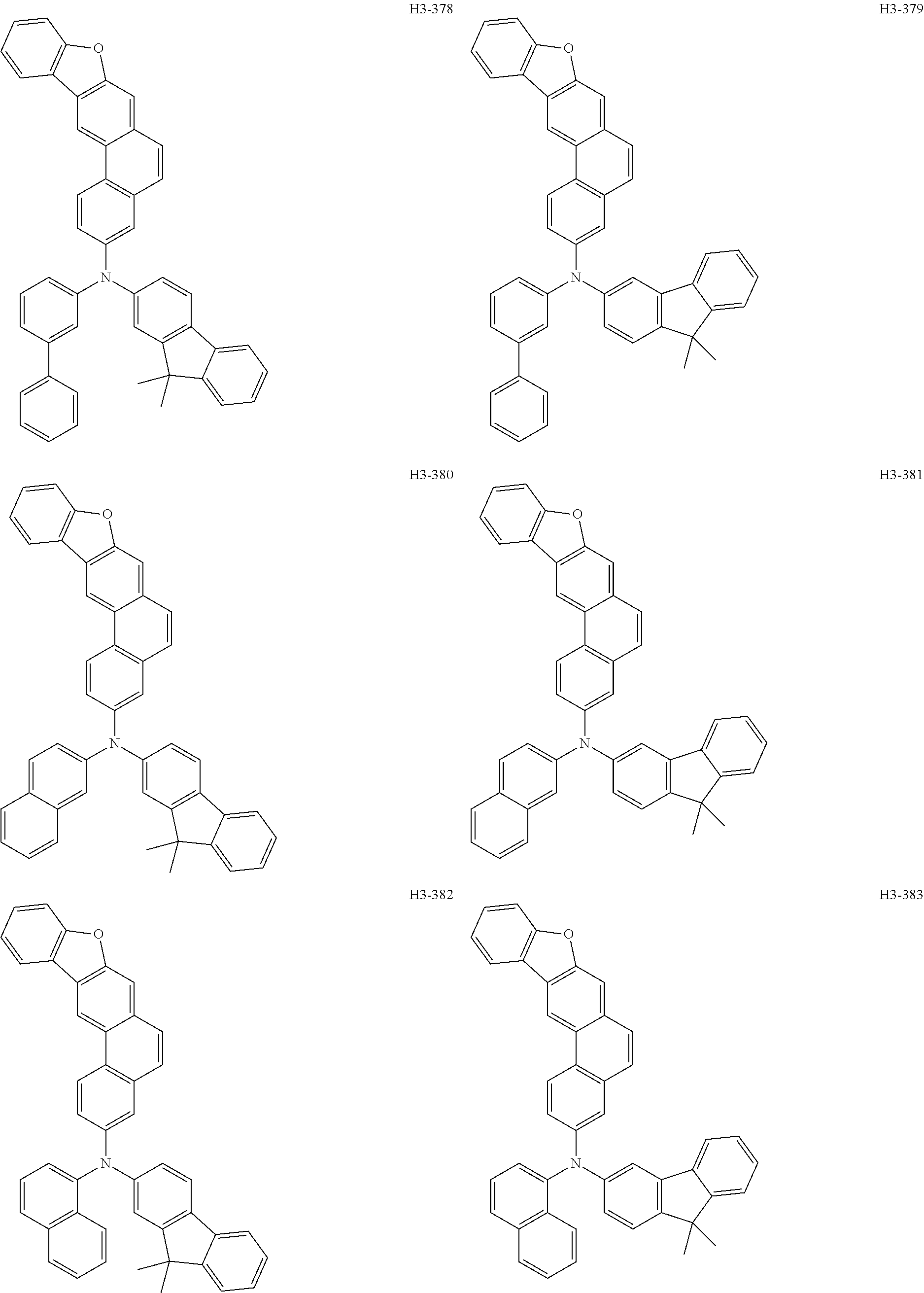

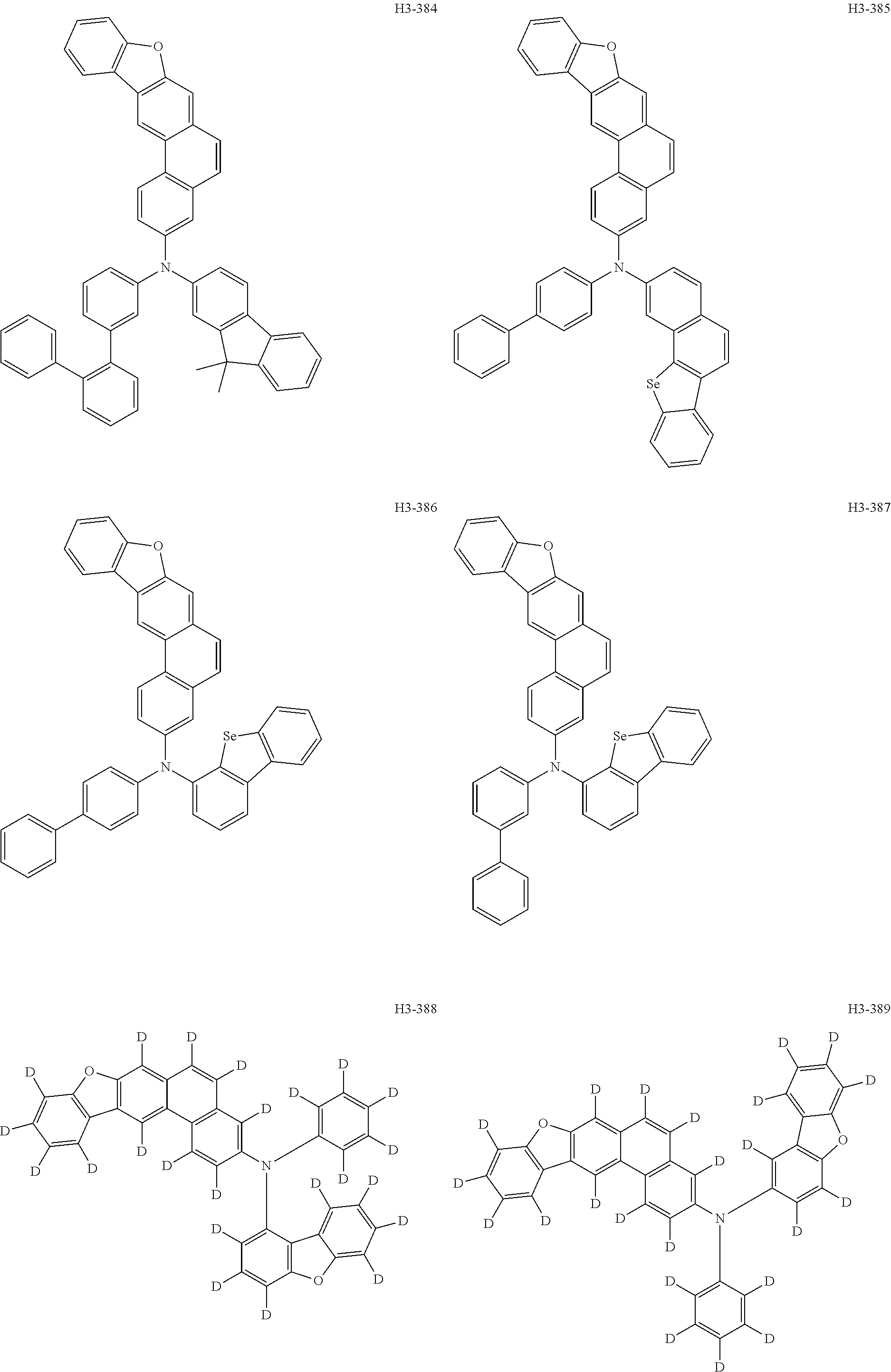

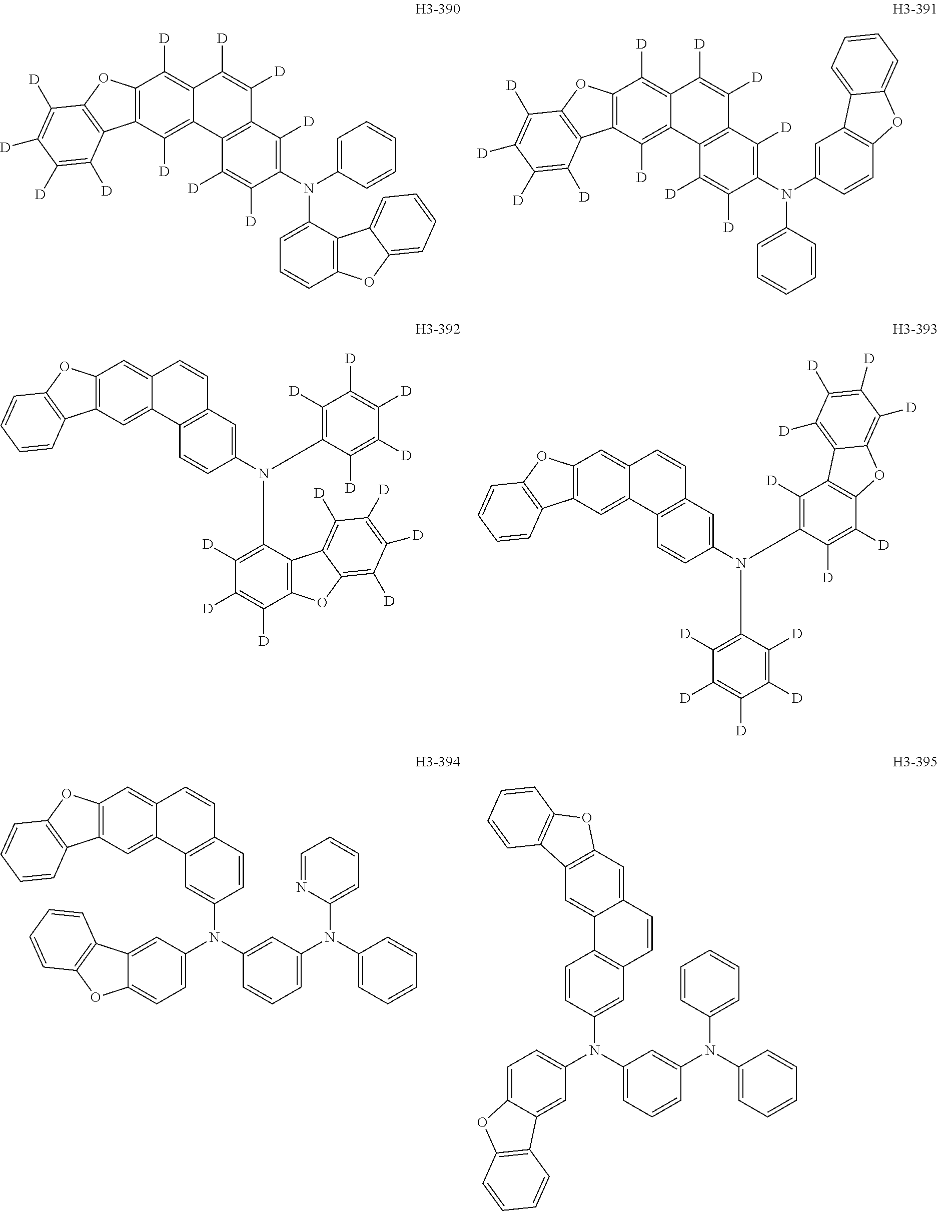

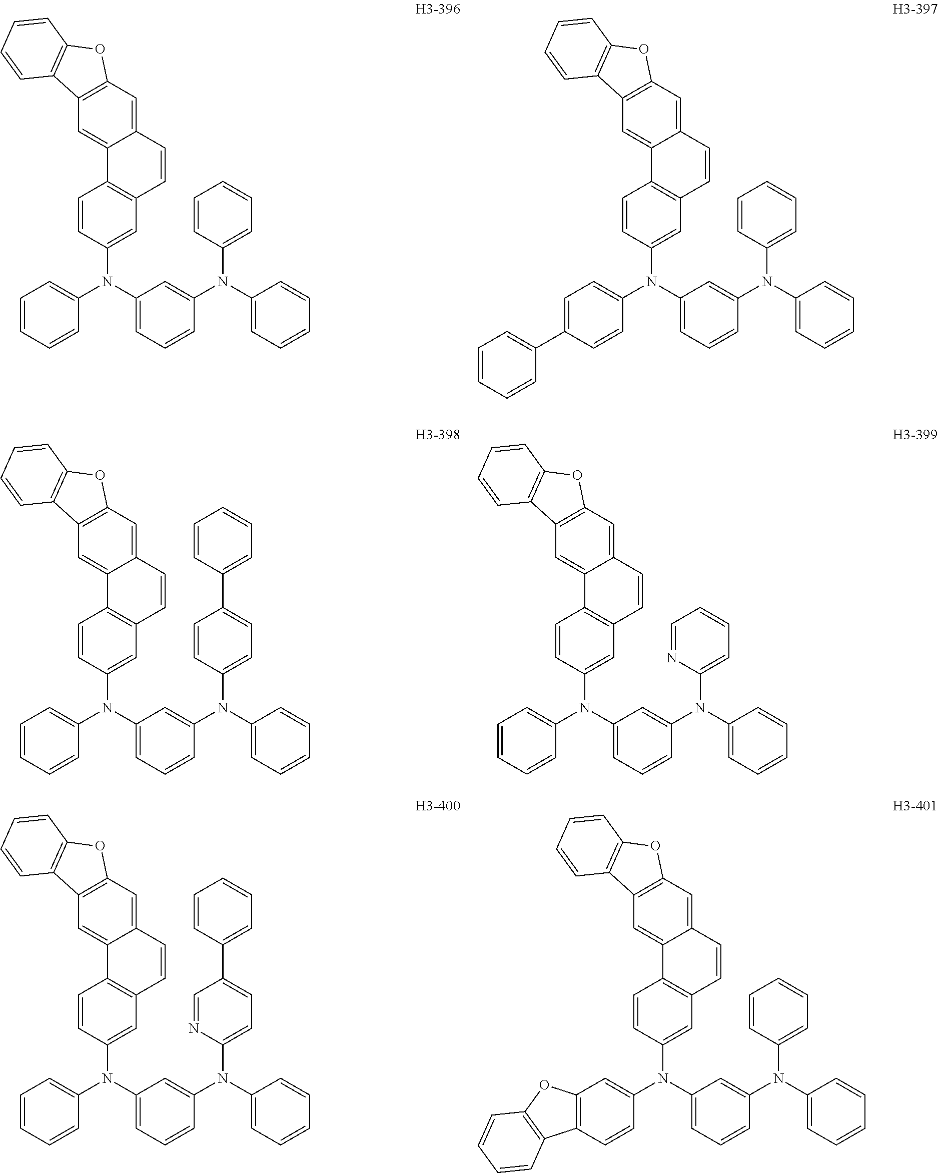

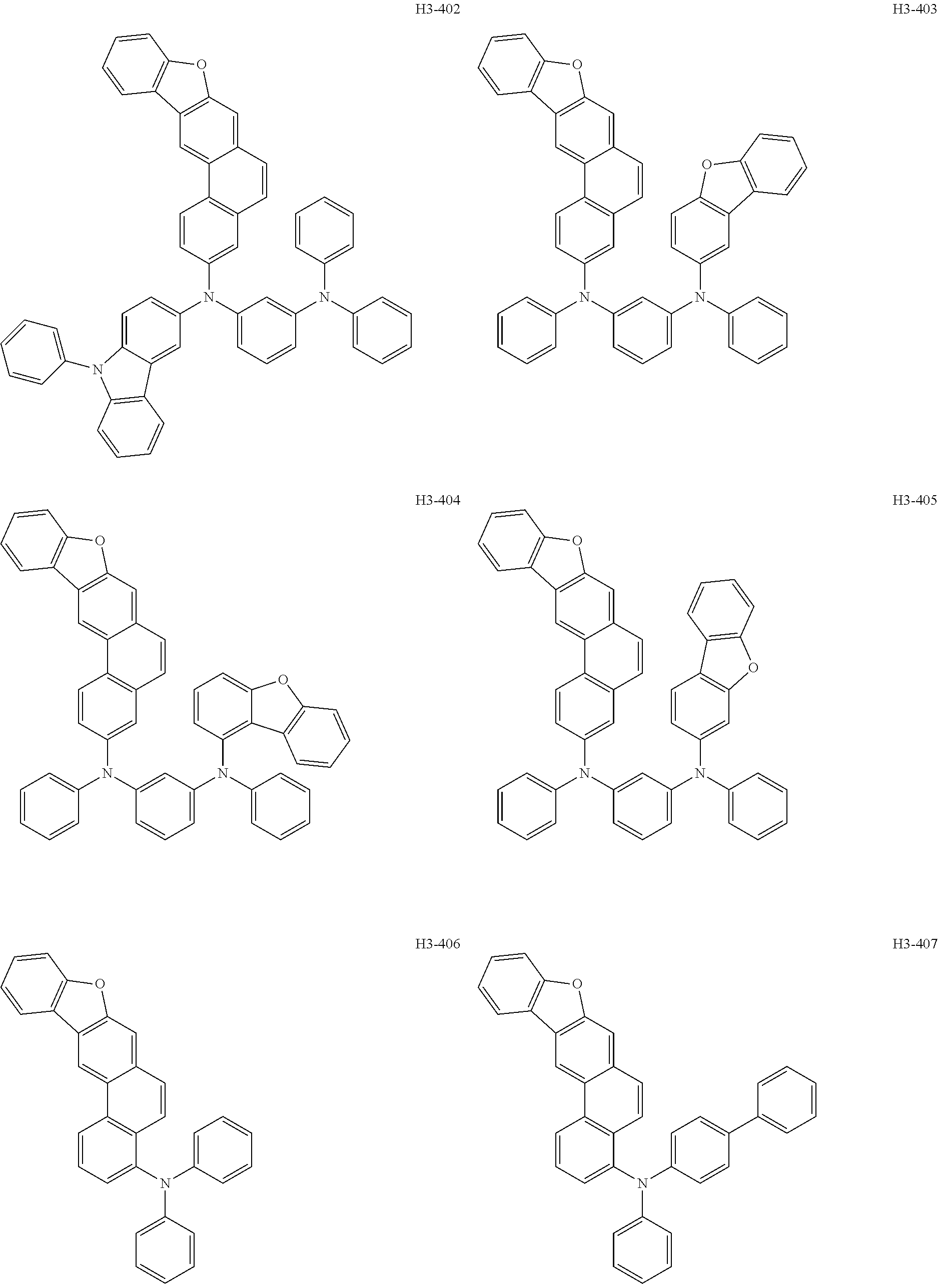 [SMILES: c1ccc(N(c2ccccc2)c2cccc(N(c3ccc4c(ccc5cc6oc7ccccc7c6cc54)c3)c3ccc4c(c3)c3ccccc3n4-c3ccccc3)c2)cc1 ; c1ccc(N(c2cccc(N(c3ccccc3)c3ccc4oc5ccccc5c4c3)c2)c2ccc3c(ccc4cc5oc6ccccc6c5cc43)c2)cc1 ; c1ccc(N(c2cccc(N(c3ccccc3)c3cccc4oc5ccccc5c34)c2)c2ccc3c(ccc4cc5oc6ccccc6c5cc43)c2)cc1 ; c1ccc(N(c2cccc(N(c3ccccc3)c3ccc4c(c3)oc3ccccc34)c2)c2ccc3c(ccc4cc5oc6ccccc6c5cc43)c2)cc1 ; c1ccc(N(c2ccccc2)c2cccc3c2ccc2cc4oc5ccccc5c4cc23)cc1 ; c1ccc(-c2ccc(N(c3ccccc3)c3cccc4c3ccc3cc5oc6ccccc6c5cc34)cc2)cc1]

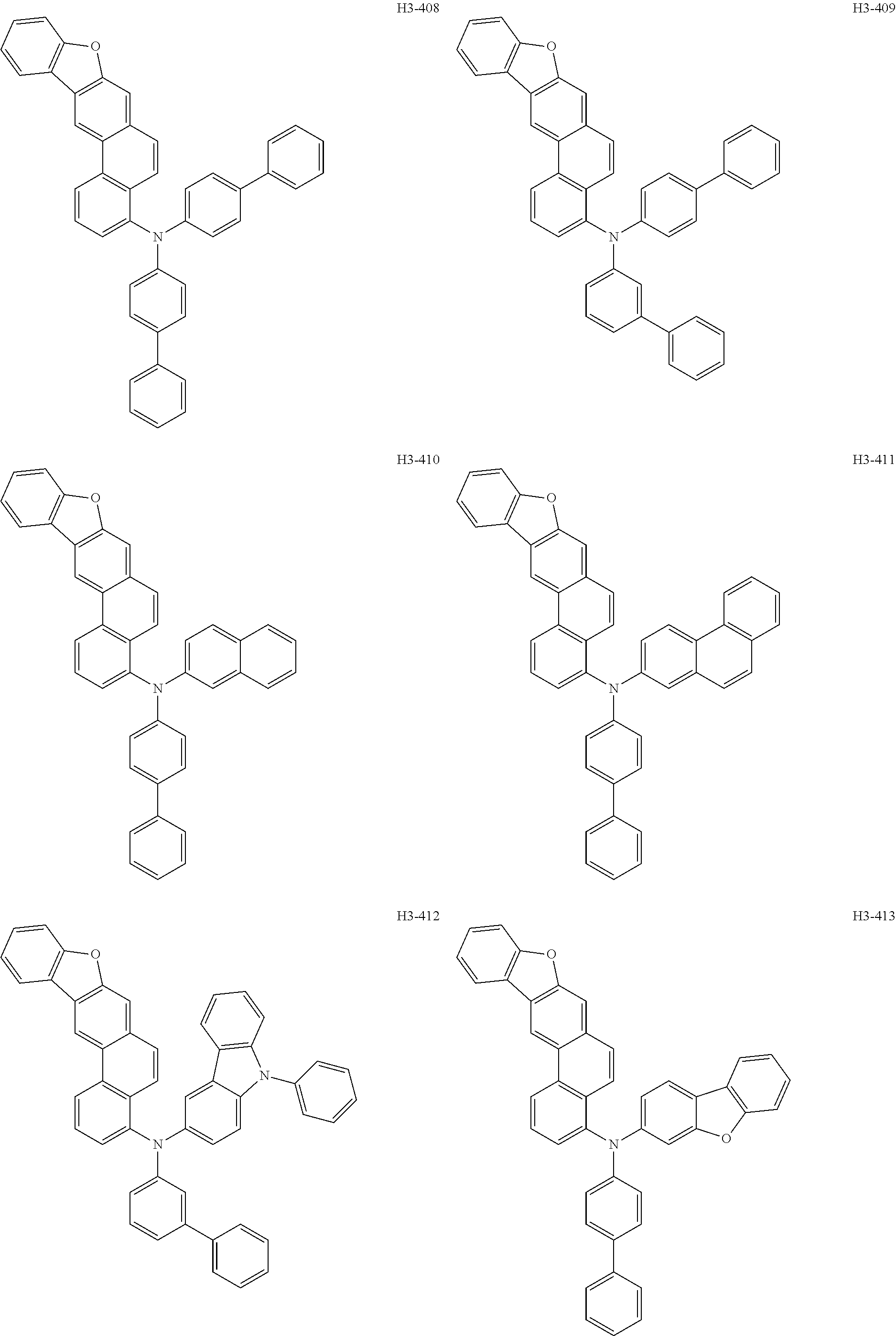 [SMILES: c1ccc(-c2ccc(N(c3ccc(-c4ccccc4)cc3)c3cccc4c3ccc3cc5oc6ccccc6c5cc34)cc2)cc1 ; c1ccc(-c2ccc(N(c3cccc(-c4ccccc4)c3)c3cccc4c3ccc3cc5oc6ccccc6c5cc34)cc2)cc1 ; c1ccc(-c2ccc(N(c3ccc4ccccc4c3)c3cccc4c3ccc3cc5oc6ccccc6c5cc34)cc2)cc1 ; c1ccc(-c2ccc(N(c3ccc4c(ccc5ccccc54)c3)c3cccc4c3ccc3cc5oc6ccccc6c5cc34)cc2)cc1 ; c1ccc(-c2cccc(N(c3ccc4c(c3)c3ccccc3n4-c3ccccc3)c3cccc4c3ccc3cc5oc6ccccc6c5cc34)c2)cc1 ; c1ccc(-c2ccc(N(c3ccc4c(c3)oc3ccccc34)c3cccc4c3ccc3cc5oc6ccccc6c5cc34)cc2)cc1]

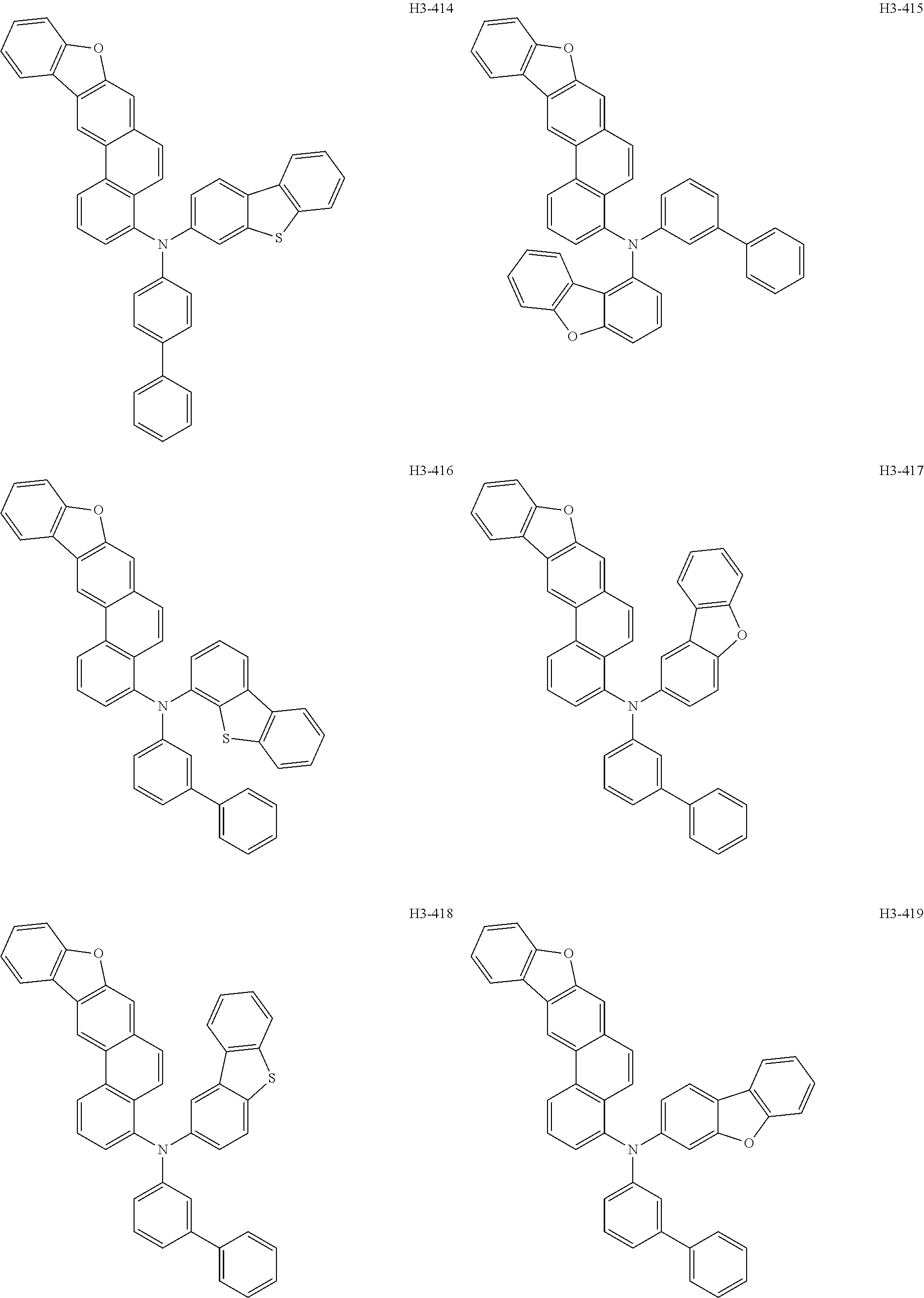 [SMILES: c1ccc(-c2ccc(N(c3ccc4c(c3)sc3ccccc34)c3cccc4c3ccc3cc5oc6ccccc6c5cc34)cc2)cc1 ; c1ccc(-c2cccc(N(c3cccc4c3ccc3cc5oc6ccccc6c5cc34)c3cccc4oc5ccccc5c34)c2)cc1 ; c1ccc(-c2cccc(N(c3cccc4c3ccc3cc5oc6ccccc6c5cc34)c3cccc4c3sc3ccccc34)c2)cc1 ; c1ccc(-c2cccc(N(c3ccc4oc5ccccc5c4c3)c3cccc4c3ccc3cc5oc6ccccc6c5cc34)c2)cc1 ; c1ccc(-c2cccc(N(c3ccc4sc5ccccc5c4c3)c3cccc4c3ccc3cc5oc6ccccc6c5cc34)c2)cc1 ; c1ccc(-c2cccc(N(c3ccc4c(c3)oc3ccccc34)c3cccc4c3ccc3cc5oc6ccccc6c5cc34)c2)cc1]

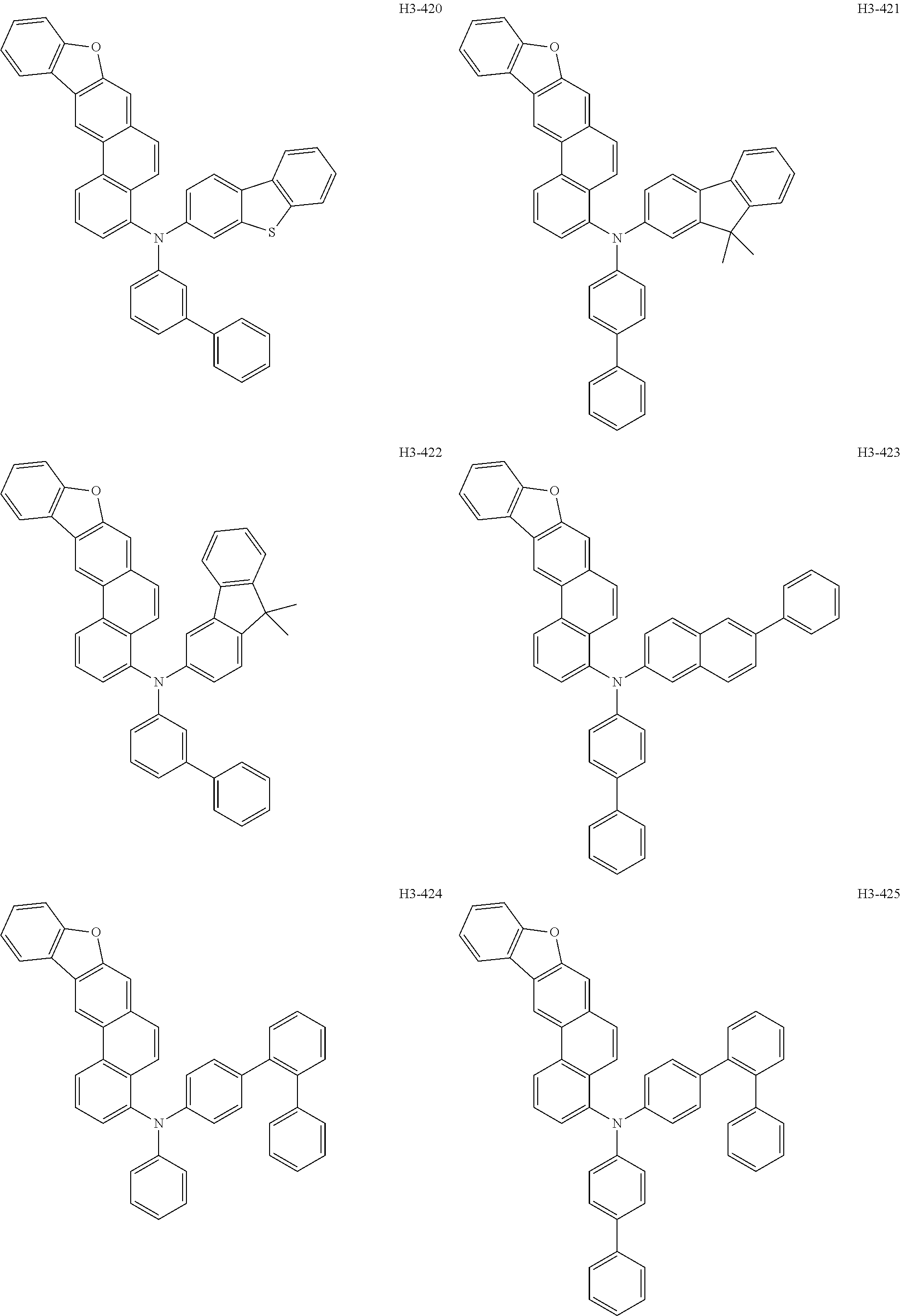 [SMILES: c1ccc(-c2cccc(N(c3ccc4c(c3)sc3ccccc34)c3cccc4c3ccc3cc5oc6ccccc6c5cc34)c2)cc1 ; CC1(C)c2ccccc2-c2ccc(N(c3ccc(-c4ccccc4)cc3)c3cccc4c3ccc3cc5oc6ccccc6c5cc34)cc21 ; CC1(C)c2ccccc2-c2cc(N(c3cccc(-c4ccccc4)c3)c3cccc4c3ccc3cc5oc6ccccc6c5cc34)ccc21 ; c1ccc(-c2ccc(N(c3ccc4cc(-c5ccccc5)ccc4c3)c3cccc4c3ccc3cc5oc6ccccc6c5cc34)cc2)cc1 ; c1ccc(-c2ccccc2-c2ccc(N(c3ccccc3)c3cccc4c3ccc3cc5oc6ccccc6c5cc34)cc2)cc1 ; c1ccc(-c2ccc(N(c3ccc(-c4ccccc4-c4ccccc4)cc3)c3cccc4c3ccc3cc5oc6ccccc6c5cc34)cc2)cc1]

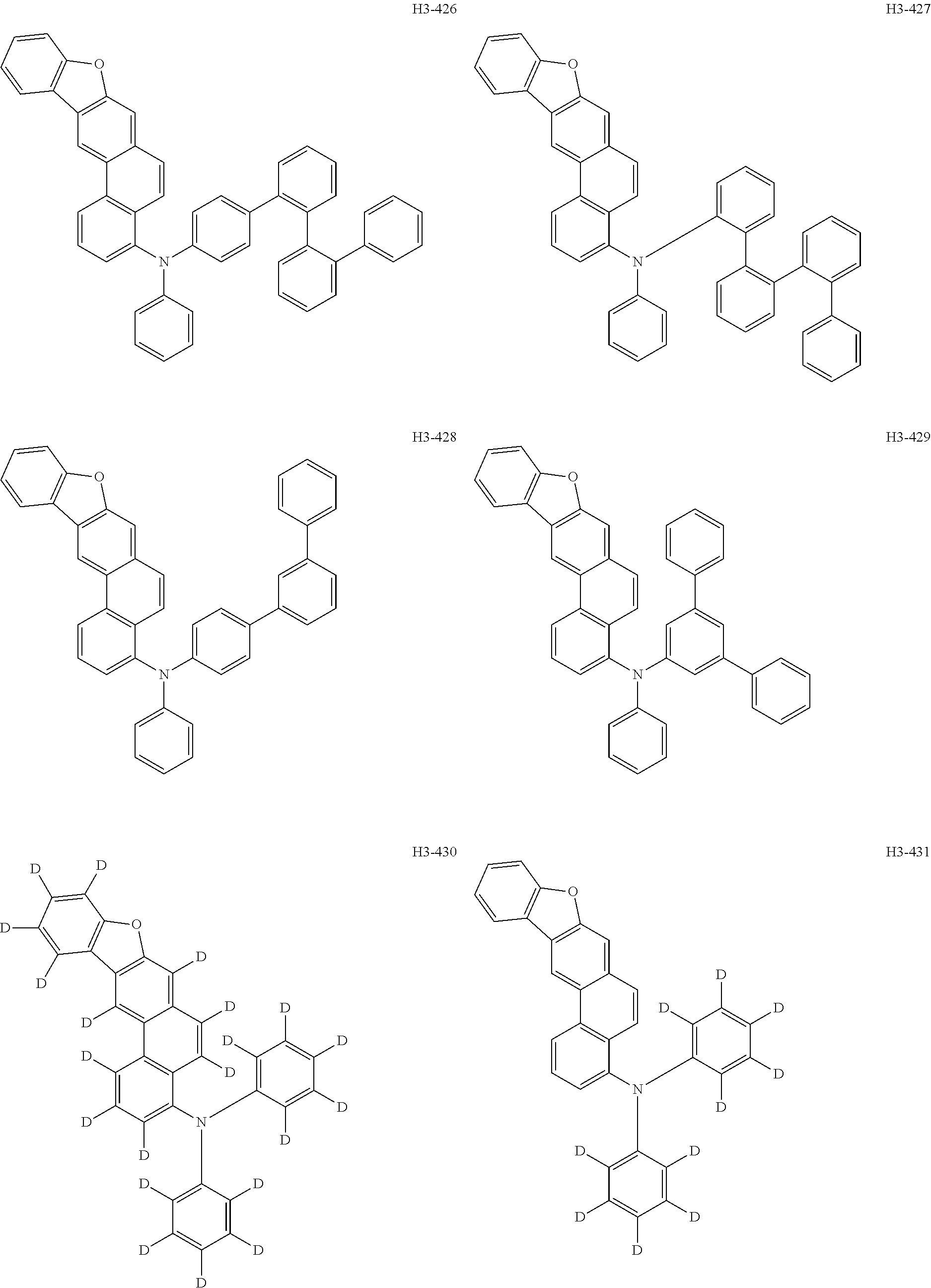

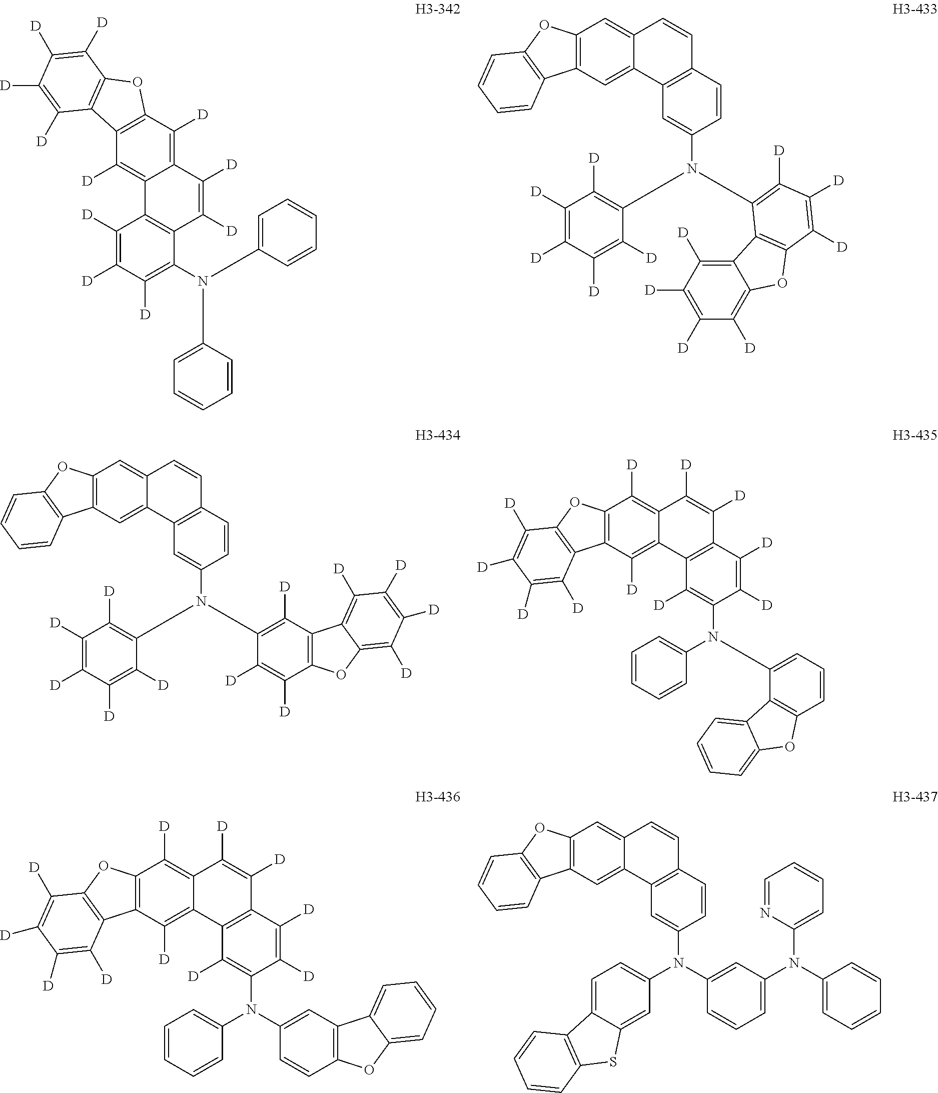

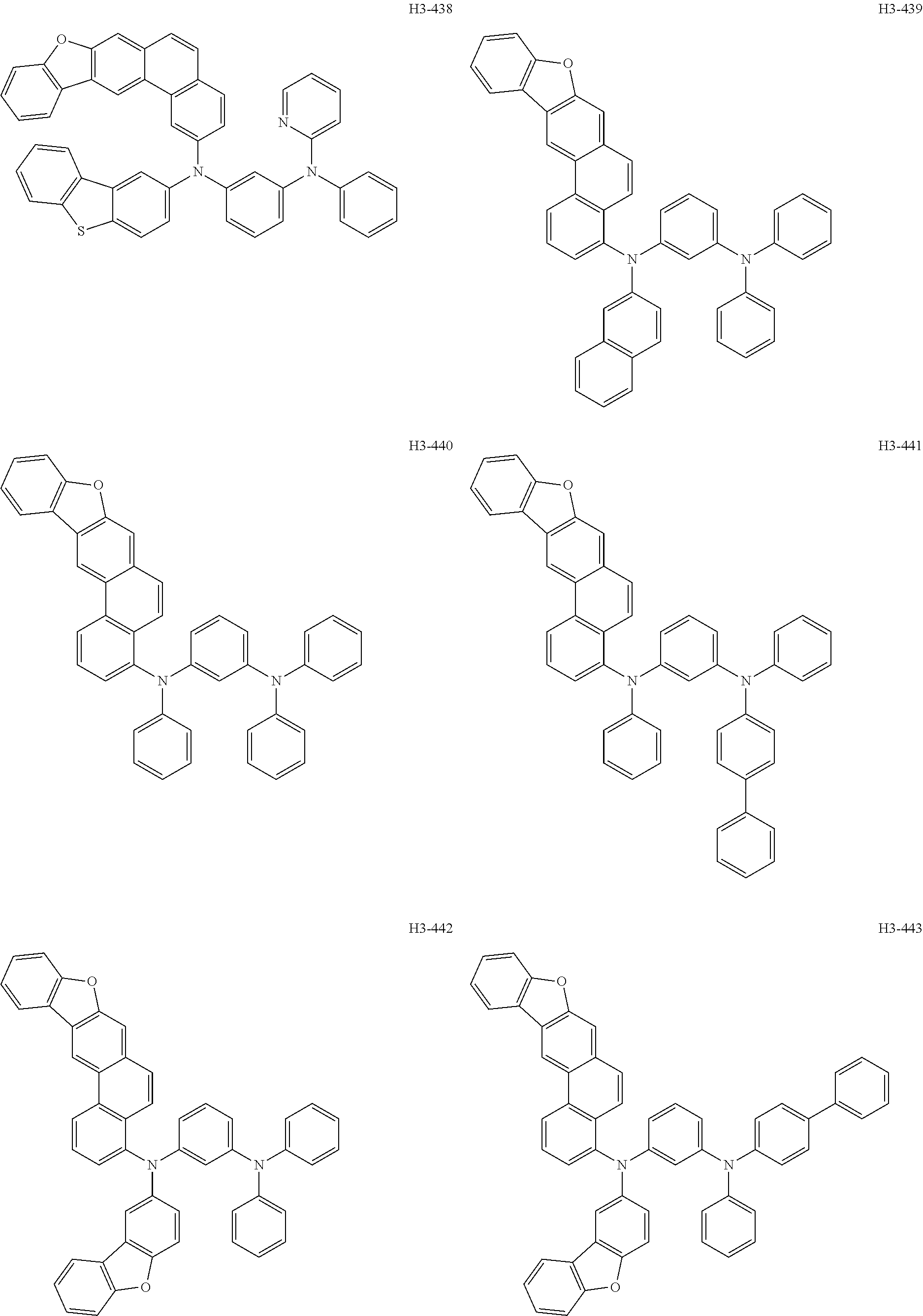

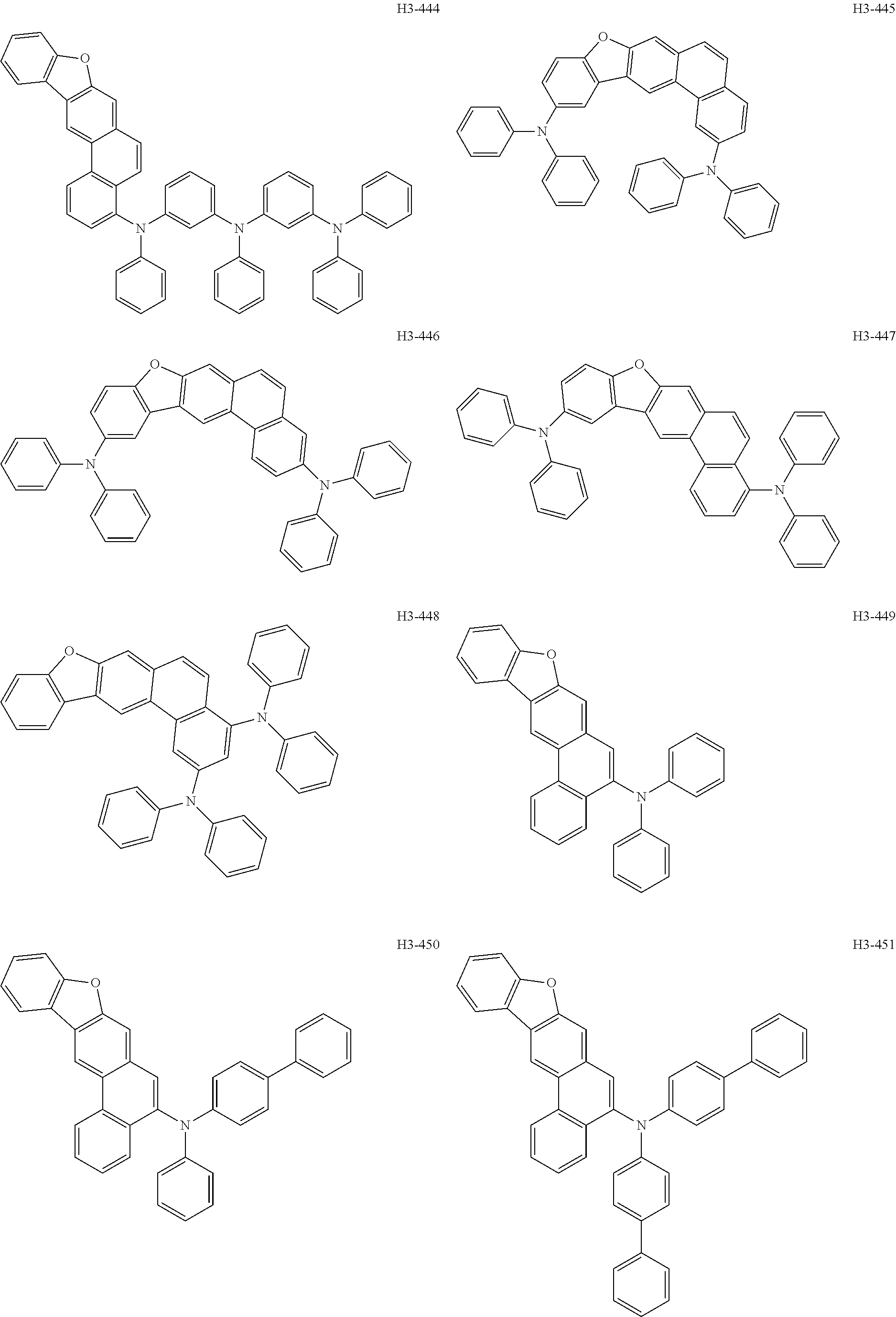

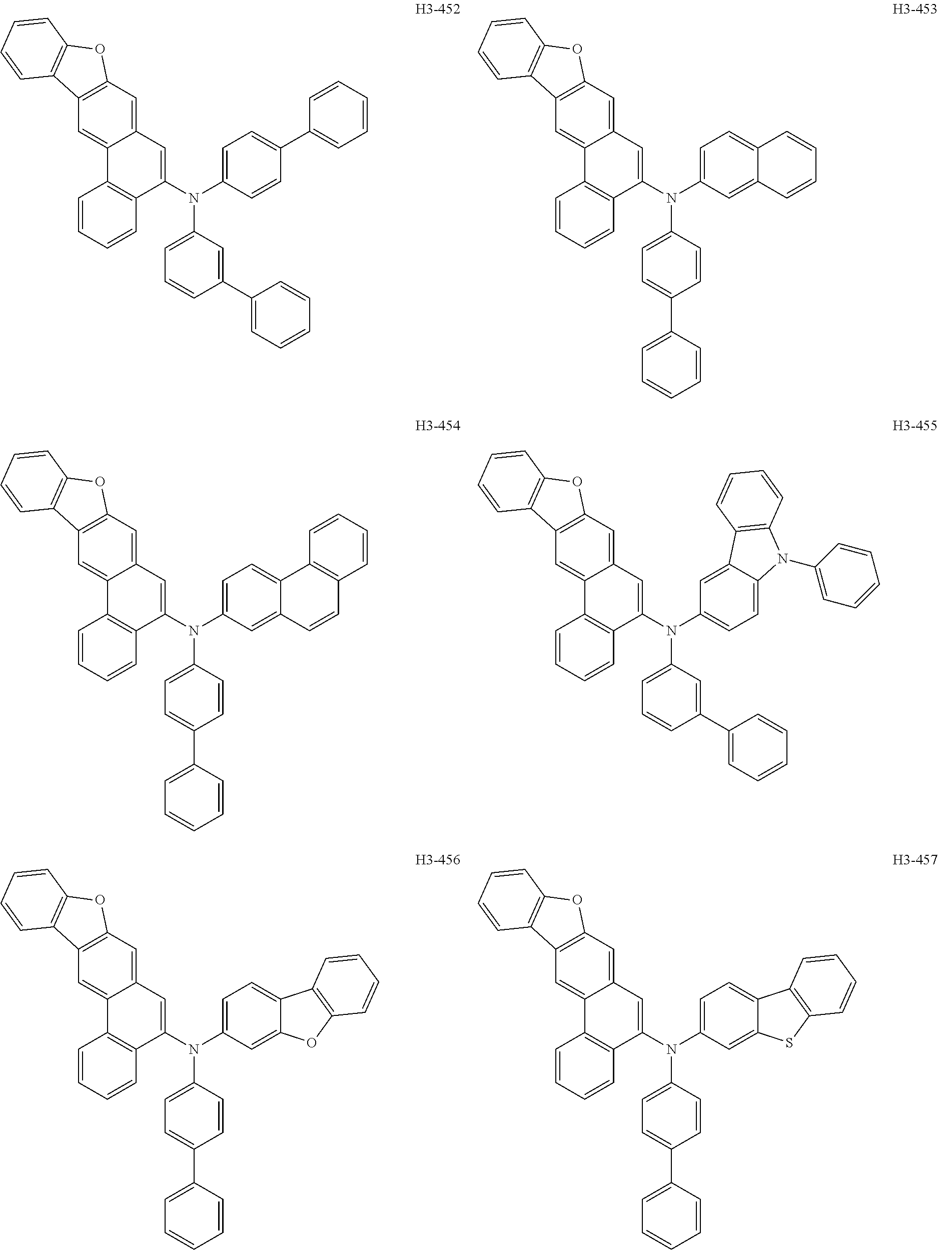 [SMILES: c1ccc(-c2ccc(N(c3cccc(-c4ccccc4)c3)c3cc4cc5oc6ccccc6c5cc4c4ccccc34)cc2)cc1 ; c1ccc(-c2ccc(N(c3ccc4ccccc4c3)c3cc4cc5oc6ccccc6c5cc4c4ccccc34)cc2)cc1 ; c1ccc(-c2ccc(N(c3ccc4c(ccc5ccccc54)c3)c3cc4cc5oc6ccccc6c5cc4c4ccccc34)cc2)cc1 ; c1ccc(-c2cccc(N(c3ccc4c(c3)c3ccccc3n4-c3ccccc3)c3cc4cc5oc6ccccc6c5cc4c4ccccc34)c2)cc1 ; c1ccc(-c2ccc(N(c3ccc4c(c3)oc3ccccc34)c3cc4cc5oc6ccccc6c5cc4c4ccccc34)cc2)cc1 ; c1ccc(-c2ccc(N(c3ccc4c(c3)sc3ccccc34)c3cc4cc5oc6ccccc6c5cc4c4ccccc34)cc2)cc1]

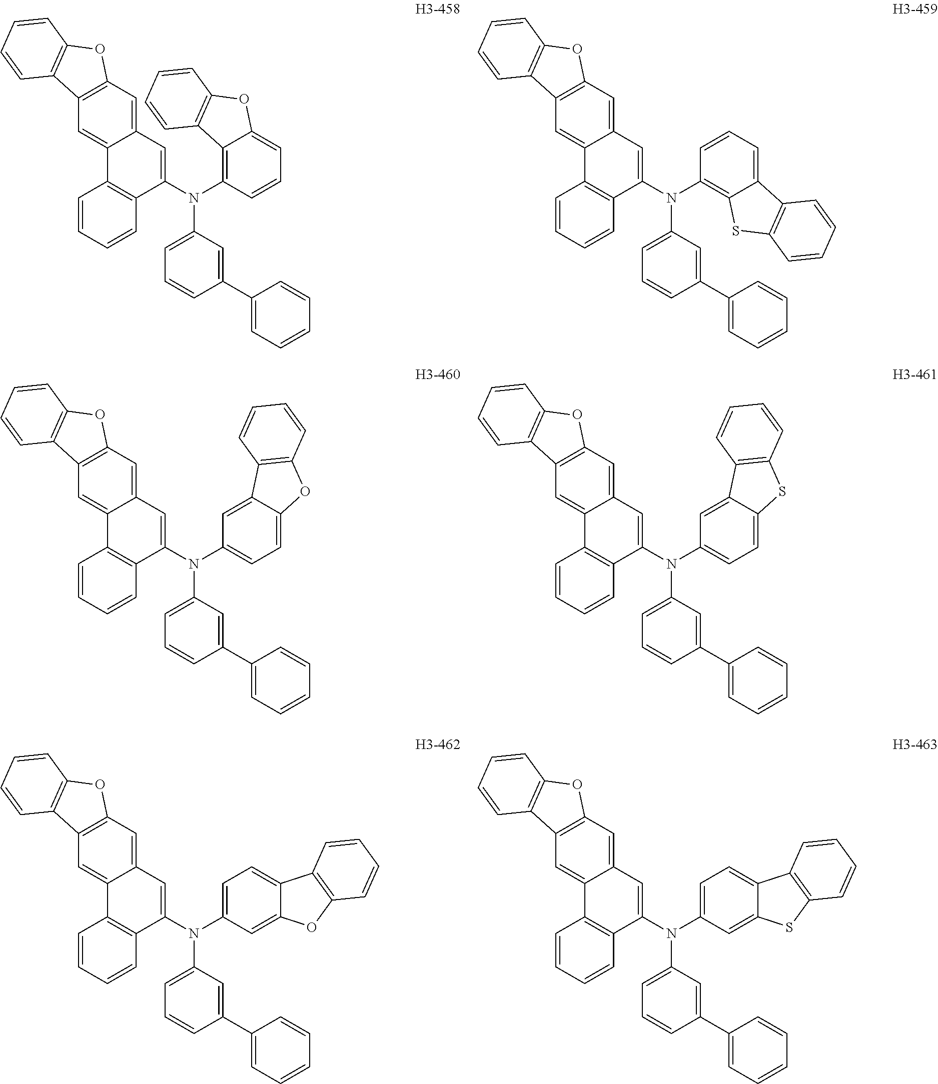 [SMILES: c1ccc(-c2cccc(N(c3cc4cc5oc6ccccc6c5cc4c4ccccc34)c3cccc4oc5ccccc5c34)c2)cc1 ; c1ccc(-c2cccc(N(c3cc4cc5oc6ccccc6c5cc4c4ccccc34)c3cccc4c3sc3ccccc34)c2)cc1 ; c1ccc(-c2cccc(N(c3ccc4oc5ccccc5c4c3)c3cc4cc5oc6ccccc6c5cc4c4ccccc34)c2)cc1 ; c1ccc(-c2cccc(N(c3ccc4sc5ccccc5c4c3)c3cc4cc5oc6ccccc6c5cc4c4ccccc34)c2)cc1 ; c1ccc(-c2cccc(N(c3ccc4c(c3)oc3ccccc34)c3cc4cc5oc6ccccc6c5cc4c4ccccc34)c2)cc1 ; c1ccc(-c2cccc(N(c3ccc4c(c3)sc3ccccc34)c3cc4cc5oc6ccccc6c5cc4c4ccccc34)c2)cc1]

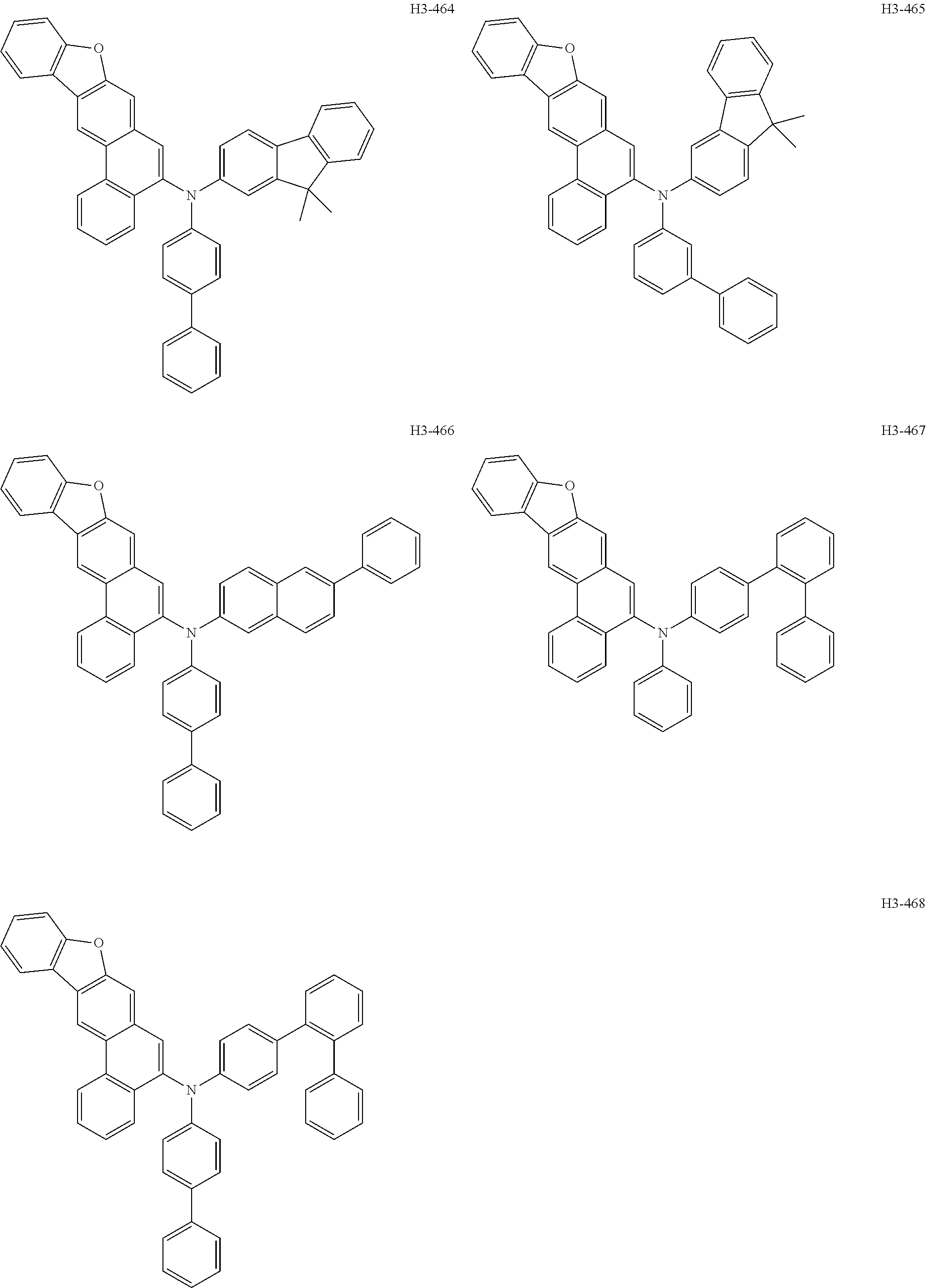 [SMILES: CC1(C)c2ccccc2-c2ccc(N(c3ccc(-c4ccccc4)cc3)c3cc4cc5oc6ccccc6c5cc4c4ccccc34)cc21 ; CC1(C)c2ccccc2-c2cc(N(c3cccc(-c4ccccc4)c3)c3cc4cc5oc6ccccc6c5cc4c4ccccc34)ccc21 ; c1ccc(-c2ccc(N(c3ccc4cc(-c5ccccc5)ccc4c3)c3cc4cc5oc6ccccc6c5cc4c4ccccc34)cc2)cc1 ; c1ccc(-c2ccccc2-c2ccc(N(c3ccccc3)c3cc4cc5oc6ccccc6c5cc4c4ccccc34)cc2)cc1 ; c1ccc(-c2ccc(N(c3ccc(-c4ccccc4-c4ccccc4)cc3)c3cc4cc5oc6ccccc6c5cc4c4ccccc34)cc2)cc1]

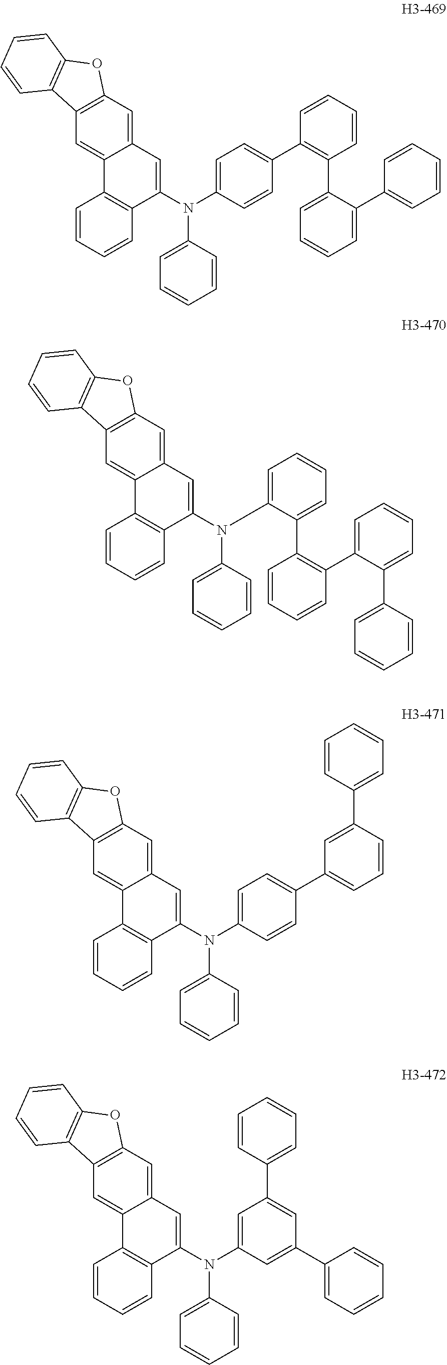 [SMILES: c1ccc(-c2ccccc2-c2ccccc2-c2ccc(N(c3ccccc3)c3cc4cc5oc6ccccc6c5cc4c4ccccc34)cc2)cc1 ; c1ccc(-c2ccccc2-c2ccccc2-c2ccccc2N(c2ccccc2)c2cc3cc4oc5ccccc5c4cc3c3ccccc23)cc1 ; c1ccc(-c2cccc(-c3ccc(N(c4ccccc4)c4cc5cc6oc7ccccc7c6cc5c5ccccc45)cc3)c2)cc1 ; c1ccc(-c2cc(-c3ccccc3)cc(N(c3ccccc3)c3cc4cc5oc6ccccc6c5cc4c4ccccc34)c2)cc1]

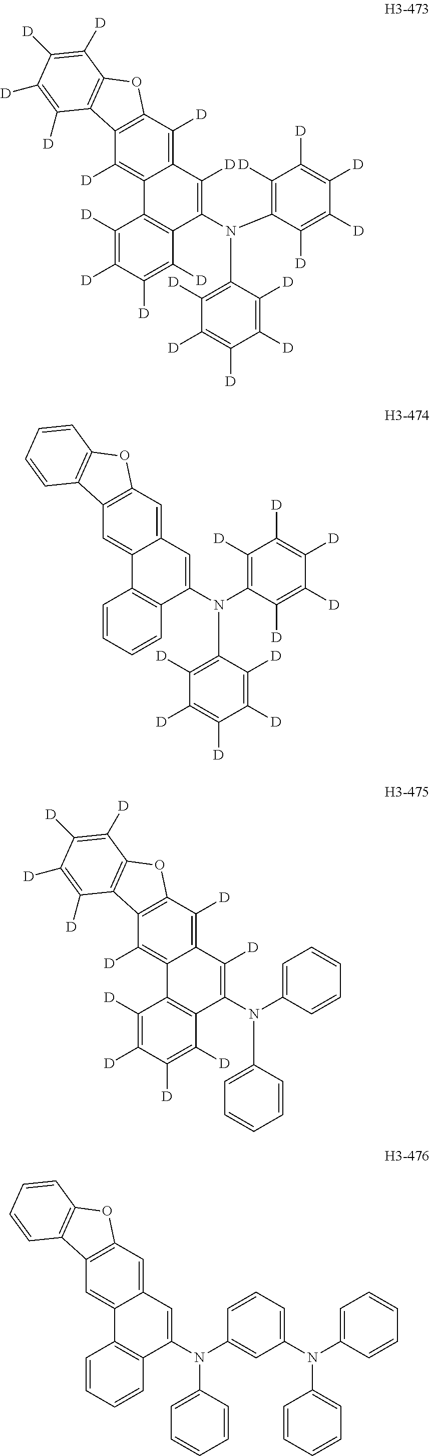 [SMILES: [2H]c1c([2H])c([2H])c(N(c2c([2H])c([2H])c([2H])c([2H])c2[2H])c2c([2H])c3c([2H])c4oc5c([2H])c([2H])c([2H])c([2H])c5c4c([2H])c3c3c([2H])c([2H])c([2H])c([2H])c23)c([2H])c1[2H] ; [2H]c1c([2H])c([2H])c(N(c2c([2H])c([2H])c([2H])c([2H])c2[2H])c2cc3cc4oc5ccccc5c4cc3c3ccccc23)c([2H])c1[2H] ; [2H]c1c([2H])c([2H])c2c(oc3c([2H])c4c([2H])c(N(c5ccccc5)c5ccccc5)c5c([2H])c([2H])c([2H])c([2H])c5c4c([2H])c32)c1[2H] ; c1ccc(N(c2ccccc2)c2cccc(N(c3ccccc3)c3cc4cc5oc6ccccc6c5cc4c4ccccc34)c2)cc1]

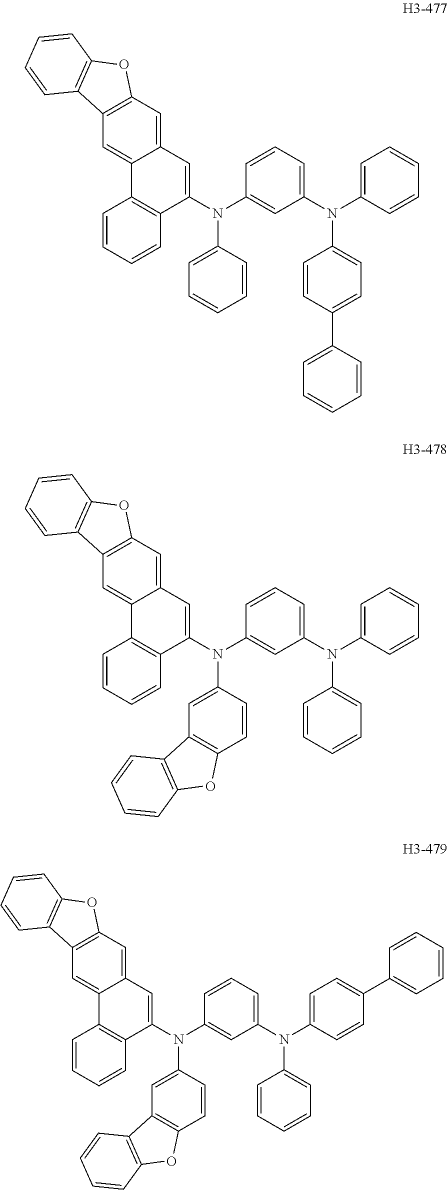 [SMILES: c1ccc(-c2ccc(N(c3ccccc3)c3cccc(N(c4ccccc4)c4cc5cc6oc7ccccc7c6cc5c5ccccc45)c3)cc2)cc1 ; c1ccc(N(c2ccccc2)c2cccc(N(c3ccc4oc5ccccc5c4c3)c3cc4cc5oc6ccccc6c5cc4c4ccccc34)c2)cc1 ; c1ccc(-c2ccc(N(c3ccccc3)c3cccc(N(c4ccc5oc6ccccc6c5c4)c4cc5cc6oc7ccccc7c6cc5c5ccccc45)c3)cc2)cc1]

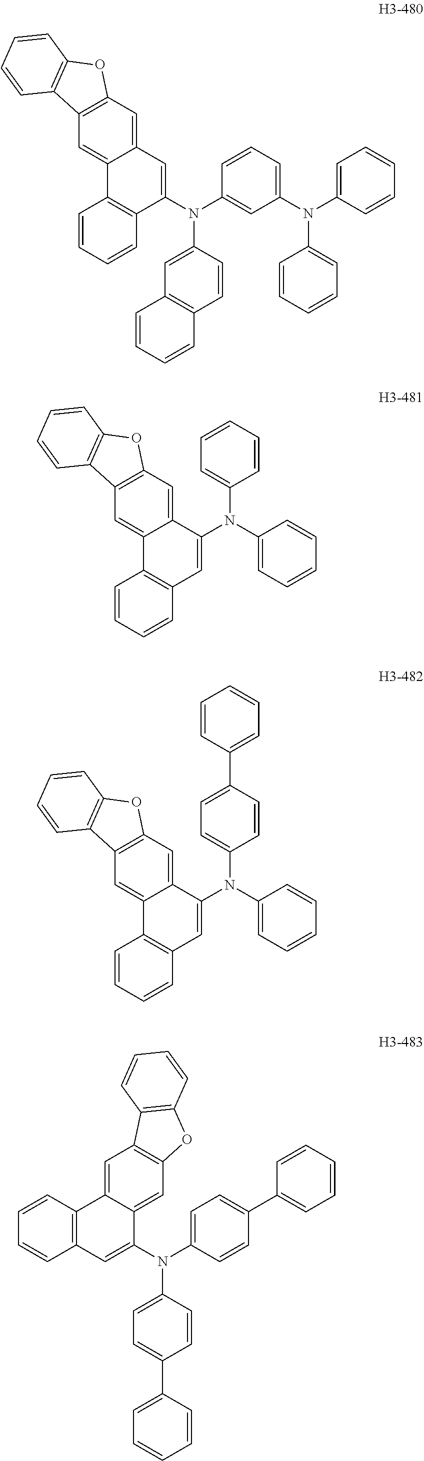 [SMILES: c1ccc(N(c2ccccc2)c2cccc(N(c3ccc4ccccc4c3)c3cc4cc5oc6ccccc6c5cc4c4ccccc34)c2)cc1 ; c1ccc(N(c2ccccc2)c2cc3ccccc3c3cc4c(cc23)oc2ccccc24)cc1 ; c1ccc(-c2ccc(N(c3ccccc3)c3cc4ccccc4c4cc5c(cc34)oc3ccccc35)cc2)cc1 ; c1ccc(-c2ccc(N(c3ccc(-c4ccccc4)cc3)c3cc4ccccc4c4cc5c(cc34)oc3ccccc35)cc2)cc1]

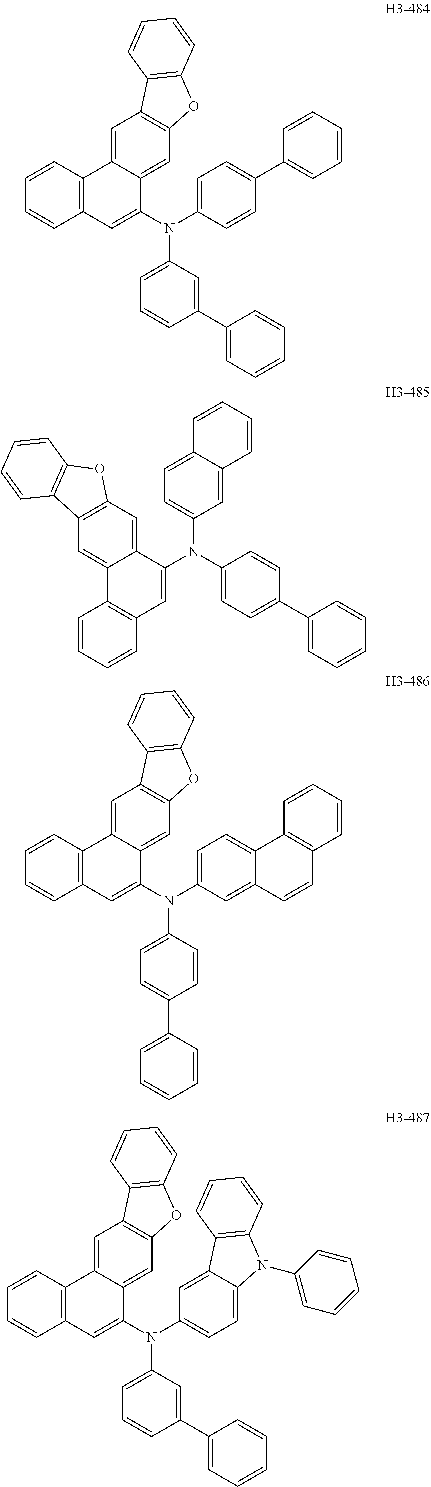

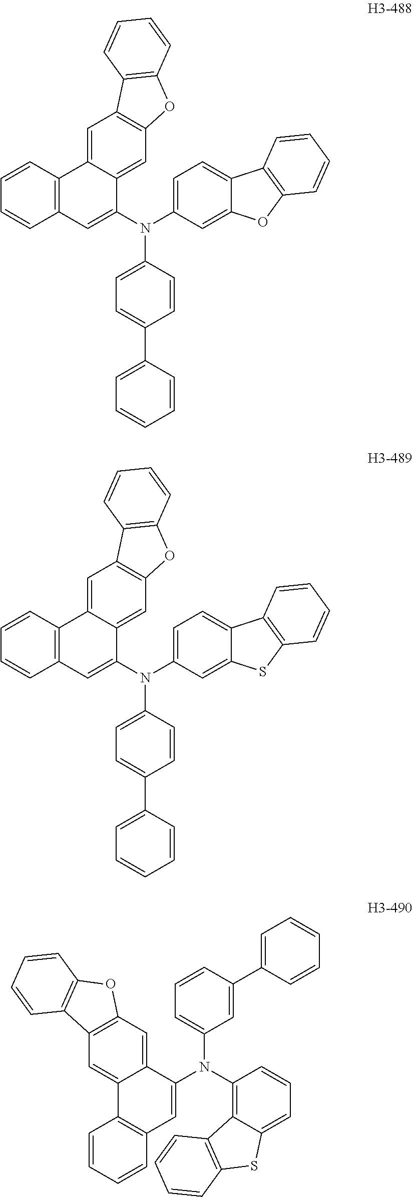

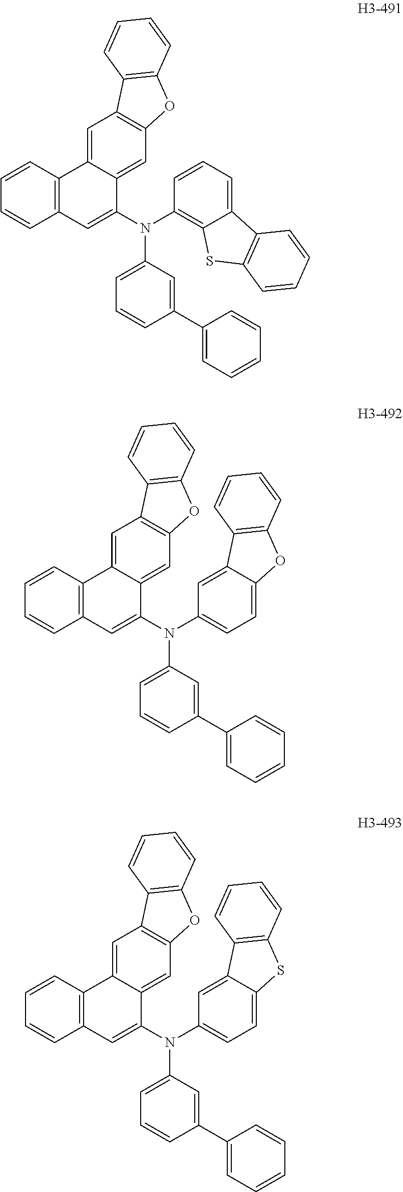 [SMILES: c1ccc(-c2cccc(N(c3cc4ccccc4c4cc5c(cc34)oc3ccccc35)c3cccc4c3sc3ccccc34)c2)cc1 ; c1ccc(-c2cccc(N(c3ccc4oc5ccccc5c4c3)c3cc4ccccc4c4cc5c(cc34)oc3ccccc35)c2)cc1 ; c1ccc(-c2cccc(N(c3ccc4sc5ccccc5c4c3)c3cc4ccccc4c4cc5c(cc34)oc3ccccc35)c2)cc1]

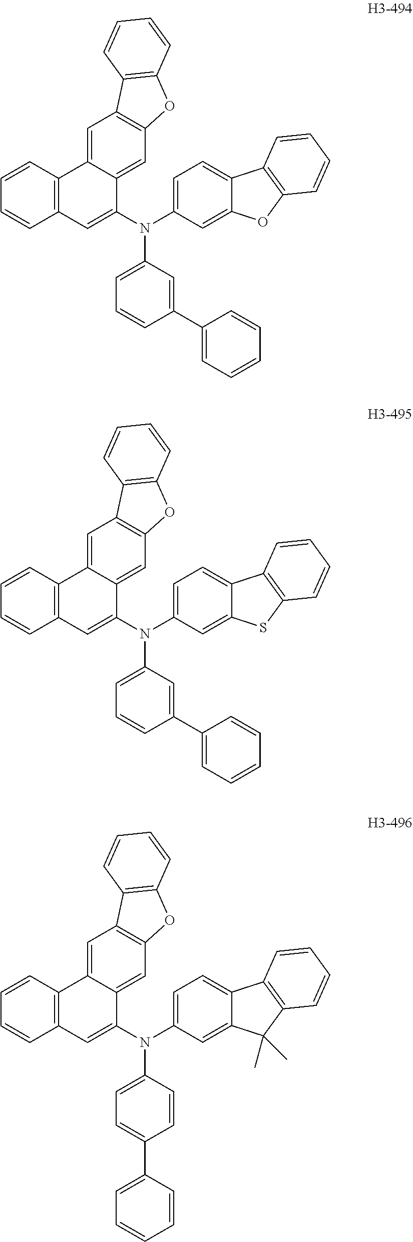 [SMILES: c1ccc(-c2cccc(N(c3ccc4c(c3)oc3ccccc34)c3cc4ccccc4c4cc5c(cc34)oc3ccccc35)c2)cc1 ; c1ccc(-c2cccc(N(c3ccc4c(c3)sc3ccccc34)c3cc4ccccc4c4cc5c(cc34)oc3ccccc35)c2)cc1 ; CC1(C)c2ccccc2-c2ccc(N(c3ccc(-c4ccccc4)cc3)c3cc4ccccc4c4cc5c(cc34)oc3ccccc35)cc21]

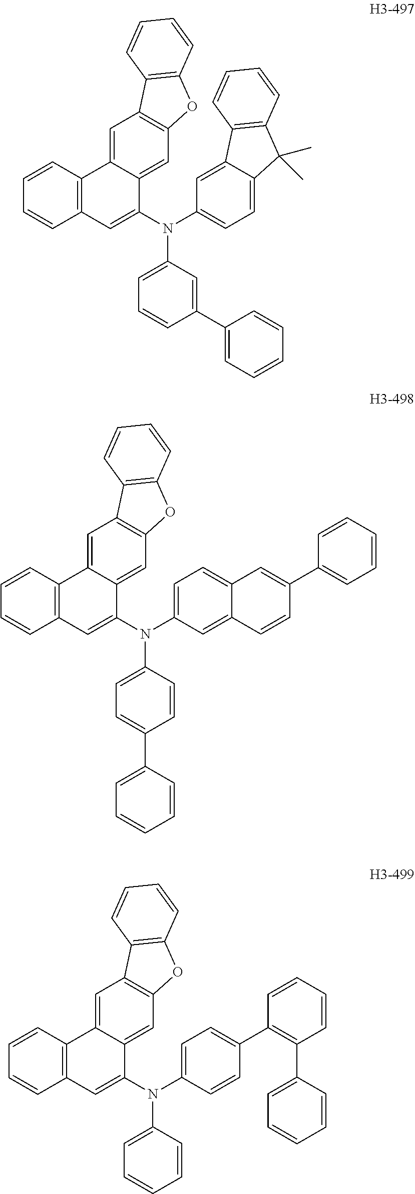 [SMILES: CC1(C)c2ccccc2-c2cc(N(c3cccc(-c4ccccc4)c3)c3cc4ccccc4c4cc5c(cc34)oc3ccccc35)ccc21 ; c1ccc(-c2ccc(N(c3ccc4cc(-c5ccccc5)ccc4c3)c3cc4ccccc4c4cc5c(cc34)oc3ccccc35)cc2)cc1 ; c1ccc(-c2ccccc2-c2ccc(N(c3ccccc3)c3cc4ccccc4c4cc5c(cc34)oc3ccccc35)cc2)cc1]

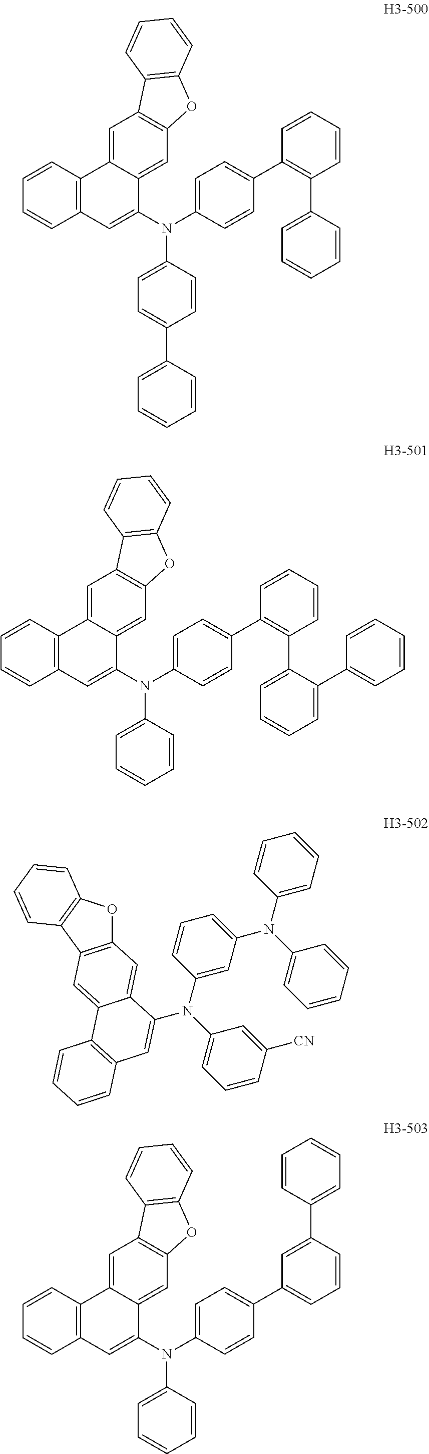 [SMILES: c1ccc(-c2ccc(N(c3ccc(-c4ccccc4-c4ccccc4)cc3)c3cc4ccccc4c4cc5c(cc34)oc3ccccc35)cc2)cc1 ; c1ccc(-c2ccccc2-c2ccccc2-c2ccc(N(c3ccccc3)c3cc4ccccc4c4cc5c(cc34)oc3ccccc35)cc2)cc1 ; N#Cc1cccc(N(c2cccc(N(c3ccccc3)c3ccccc3)c2)c2cc3ccccc3c3cc4c(cc23)oc2ccccc24)c1 ; c1ccc(-c2cccc(-c3ccc(N(c4ccccc4)c4cc5ccccc5c5cc6c(cc45)oc4ccccc46)cc3)c2)cc1]

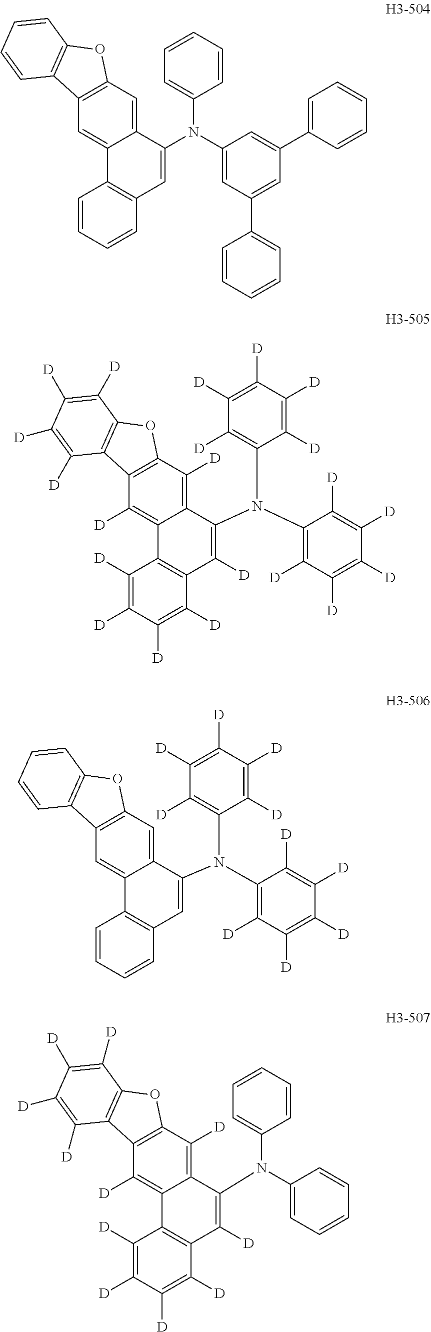 [SMILES: c1ccc(-c2cc(-c3ccccc3)cc(N(c3ccccc3)c3cc4ccccc4c4cc5c(cc34)oc3ccccc35)c2)cc1 ; [2H]c1c([2H])c([2H])c(N(c2c([2H])c([2H])c([2H])c([2H])c2[2H])c2c([2H])c3c([2H])c([2H])c([2H])c([2H])c3c3c([2H])c4c(oc5c([2H])c([2H])c([2H])c([2H])c54)c([2H])c23)c([2H])c1[2H] ; [2H]c1c([2H])c([2H])c(N(c2c([2H])c([2H])c([2H])c([2H])c2[2H])c2cc3ccccc3c3cc4c(cc23)oc2ccccc24)c([2H])c1[2H] ; [2H]c1c([2H])c([2H])c2c(oc3c([2H])c4c(N(c5ccccc5)c5ccccc5)c([2H])c5c([2H])c([2H])c([2H])c([2H])c5c4c([2H])c32)c1[2H]]

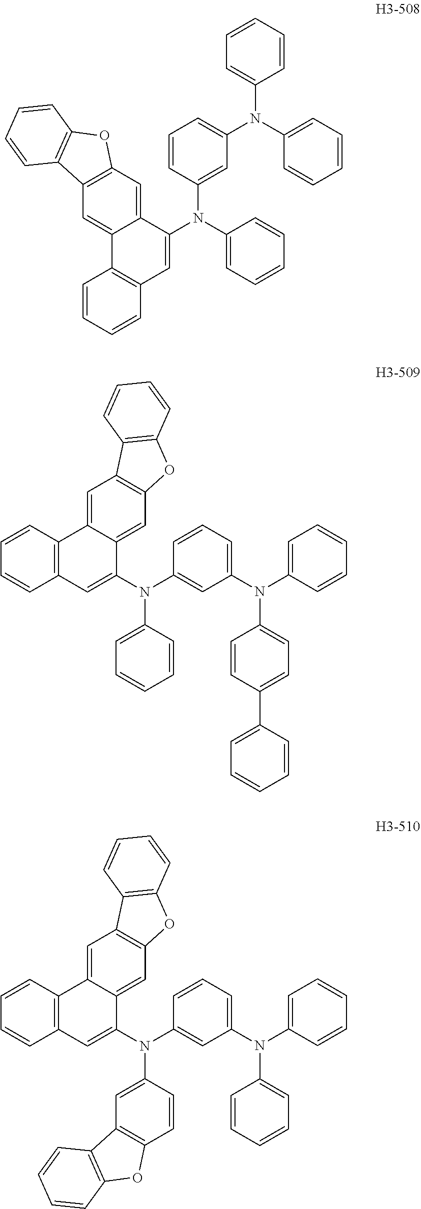 [SMILES: c1ccc(N(c2ccccc2)c2cccc(N(c3ccccc3)c3cc4ccccc4c4cc5c(cc34)oc3ccccc35)c2)cc1 ; c1ccc(-c2ccc(N(c3ccccc3)c3cccc(N(c4ccccc4)c4cc5ccccc5c5cc6c(cc45)oc4ccccc46)c3)cc2)cc1 ; c1ccc(N(c2ccccc2)c2cccc(N(c3ccc4oc5ccccc5c4c3)c3cc4ccccc4c4cc5c(cc34)oc3ccccc35)c2)cc1]

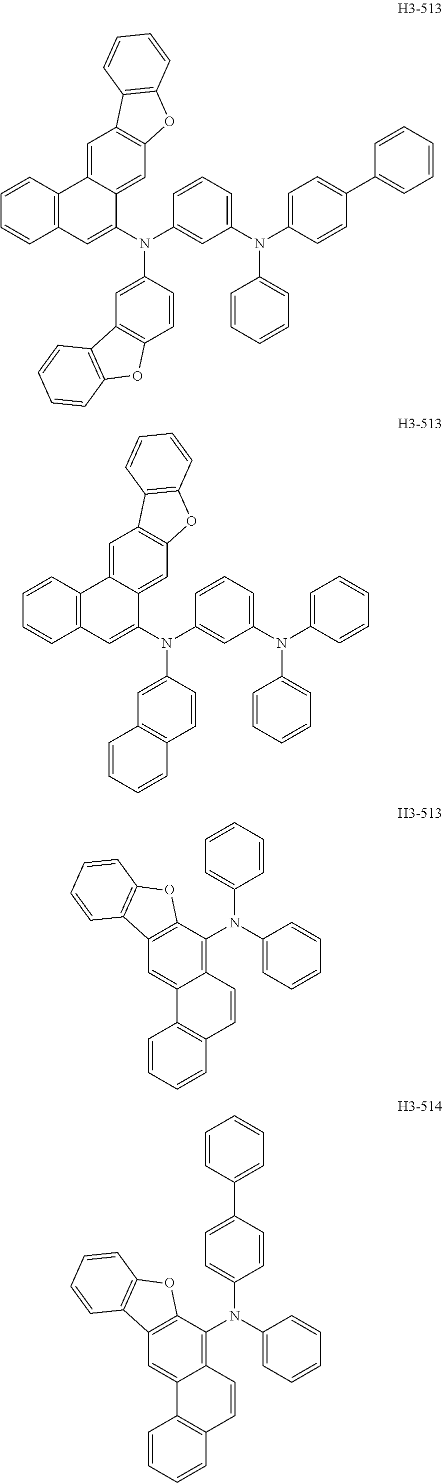 [SMILES: c1ccc(-c2ccc(N(c3ccccc3)c3cccc(N(c4ccc5oc6ccccc6c5c4)c4cc5ccccc5c5cc6c(cc45)oc4ccccc46)c3)cc2)cc1 ; c1ccc(N(c2ccccc2)c2cccc(N(c3ccc4ccccc4c3)c3cc4ccccc4c4cc5c(cc34)oc3ccccc35)c2)cc1 ; c1ccc(N(c2ccccc2)c2c3ccc4ccccc4c3cc3c2oc2ccccc23)cc1 ; c1ccc(-c2ccc(N(c3ccccc3)c3c4ccc5ccccc5c4cc4c3oc3ccccc34)cc2)cc1]

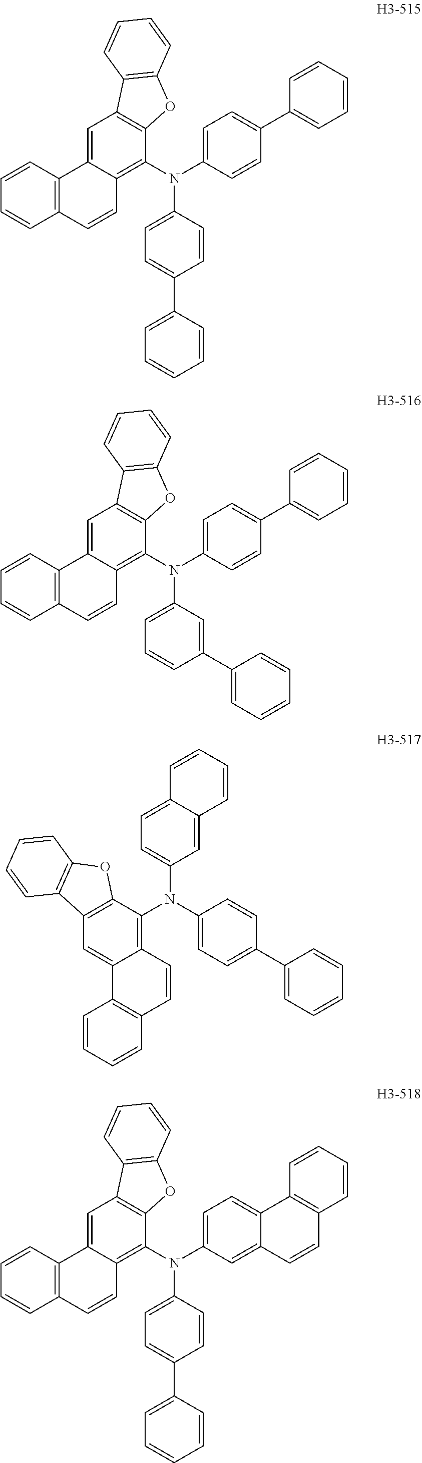

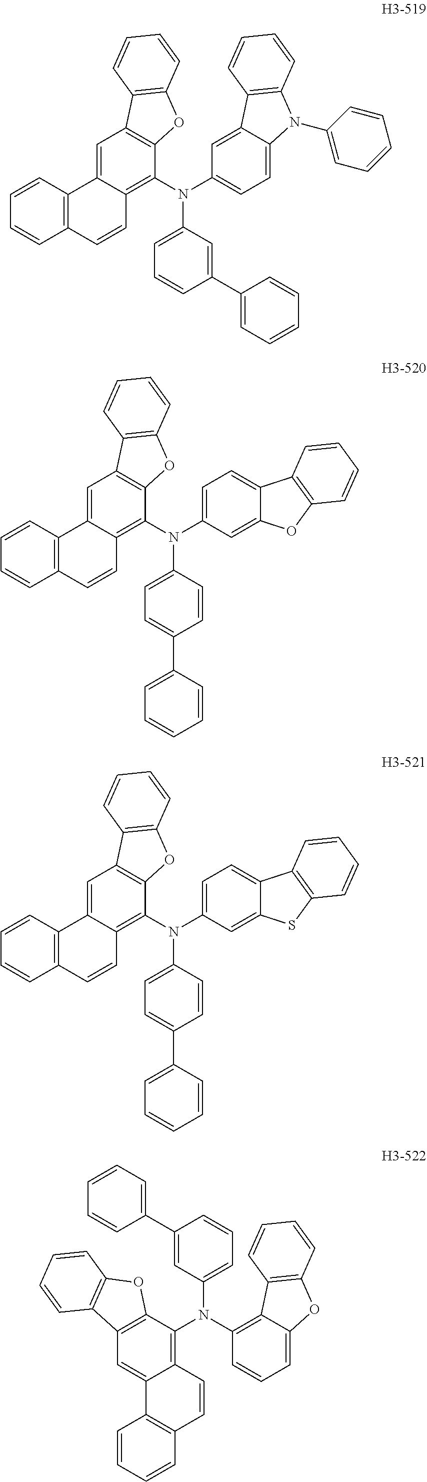

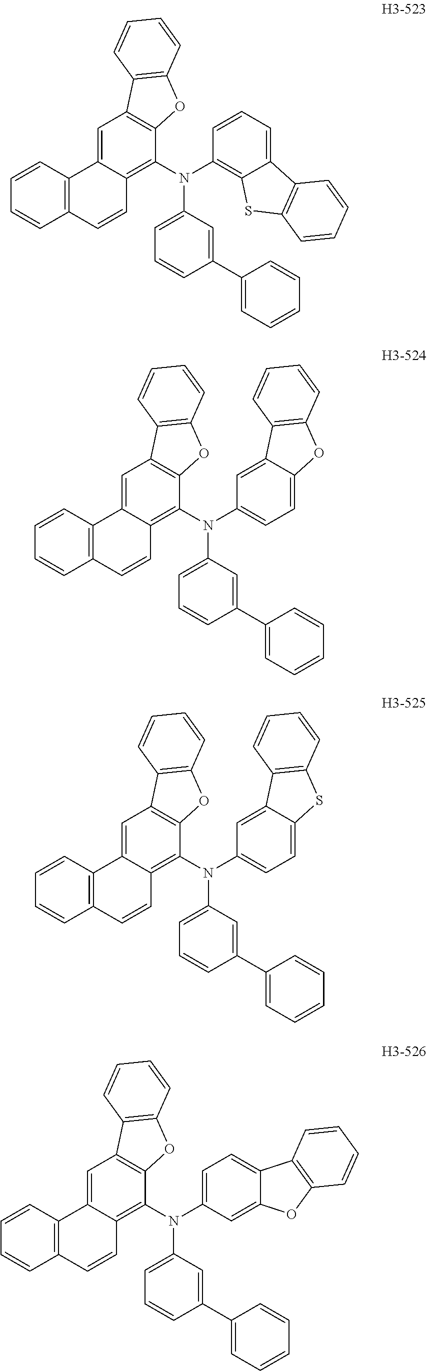

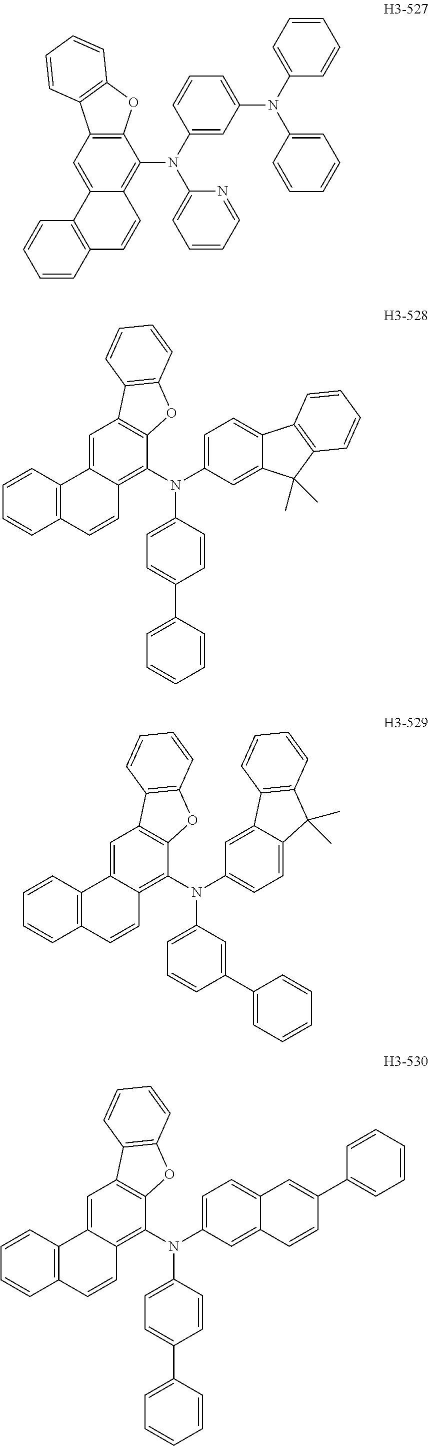 [SMILES: c1ccc(N(c2ccccc2)c2cccc(N(c3ccccn3)c3c4ccc5ccccc5c4cc4c3oc3ccccc34)c2)cc1 ; CC1(C)c2ccccc2-c2ccc(N(c3ccc(-c4ccccc4)cc3)c3c4ccc5ccccc5c4cc4c3oc3ccccc34)cc21 ; CC1(C)c2ccccc2-c2cc(N(c3cccc(-c4ccccc4)c3)c3c4ccc5ccccc5c4cc4c3oc3ccccc34)ccc21 ; c1ccc(-c2ccc(N(c3ccc4cc(-c5ccccc5)ccc4c3)c3c4ccc5ccccc5c4cc4c3oc3ccccc34)cc2)cc1]

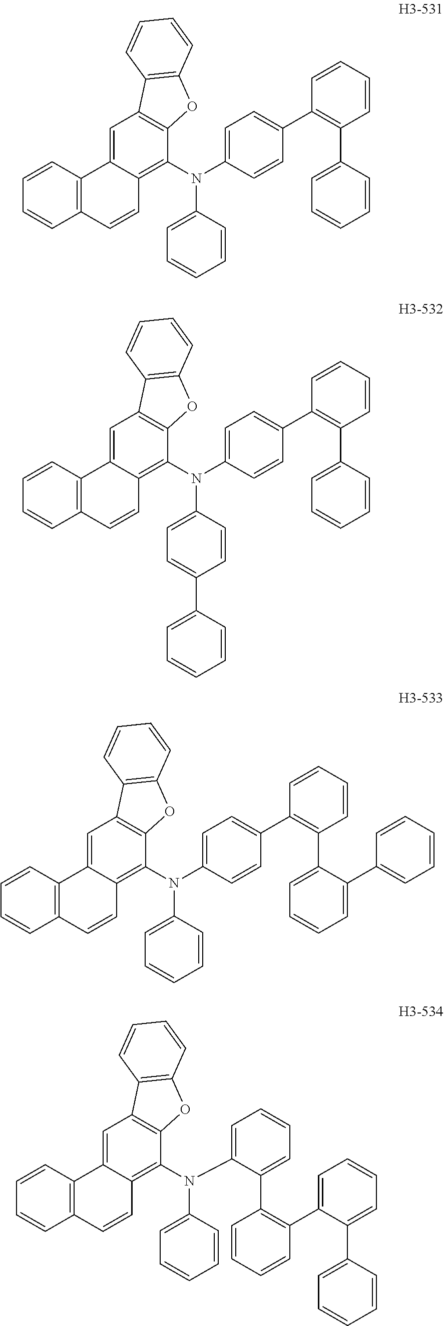

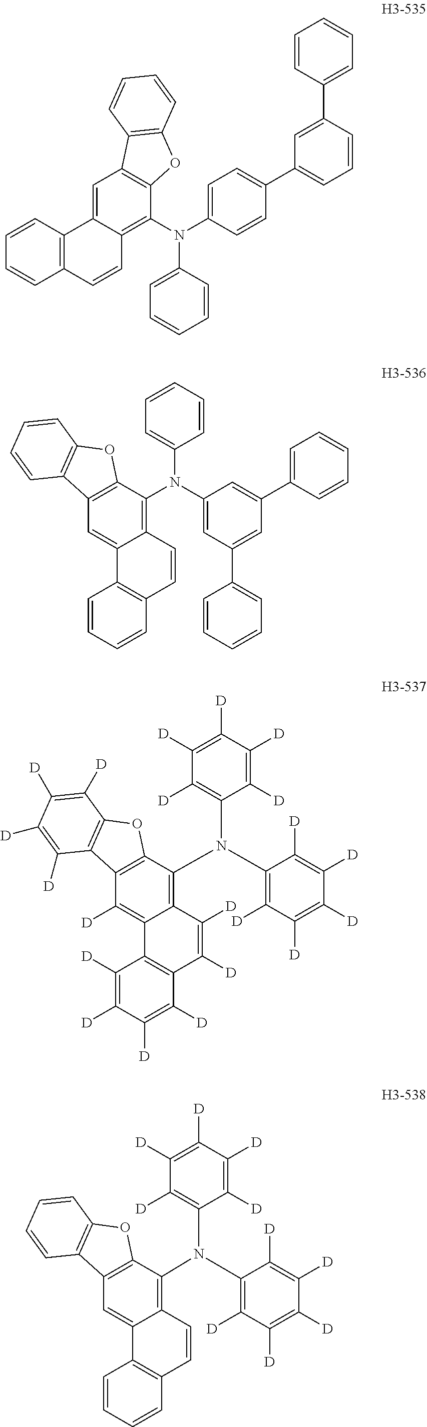 [SMILES: c1ccc(-c2cccc(-c3ccc(N(c4ccccc4)c4c5ccc6ccccc6c5cc5c4oc4ccccc45)cc3)c2)cc1 ; c1ccc(-c2cc(-c3ccccc3)cc(N(c3ccccc3)c3c4ccc5ccccc5c4cc4c3oc3ccccc34)c2)cc1 ; [2H]c1c([2H])c([2H])c(N(c2c([2H])c([2H])c([2H])c([2H])c2[2H])c2c3oc4c([2H])c([2H])c([2H])c([2H])c4c3c([2H])c3c2c([2H])c([2H])c2c([2H])c([2H])c([2H])c([2H])c23)c([2H])c1[2H] ; [2H]c1c([2H])c([2H])c(N(c2c([2H])c([2H])c([2H])c([2H])c2[2H])c2c3ccc4ccccc4c3cc3c2oc2ccccc23)c([2H])c1[2H]]

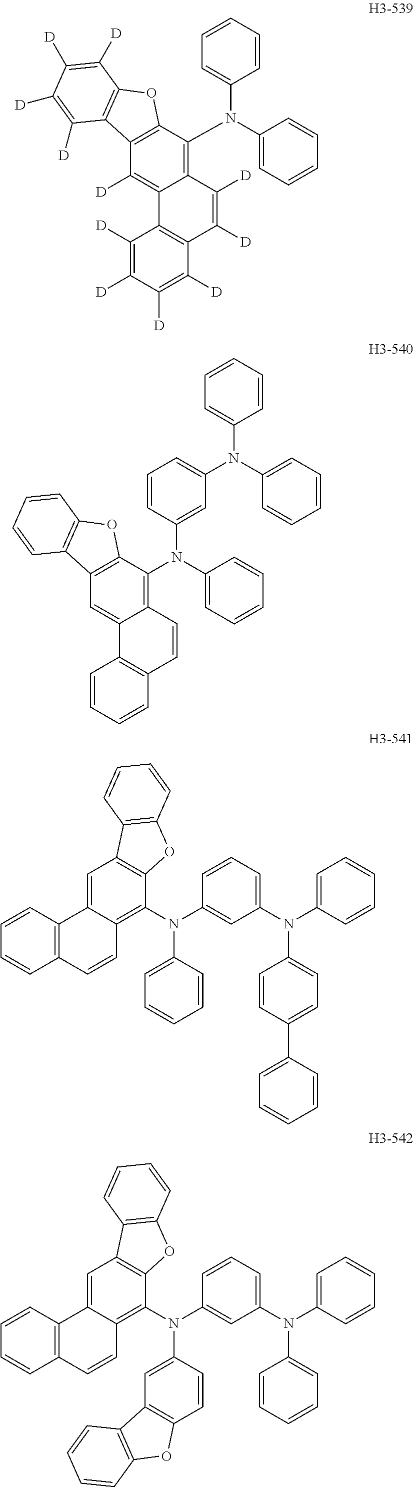

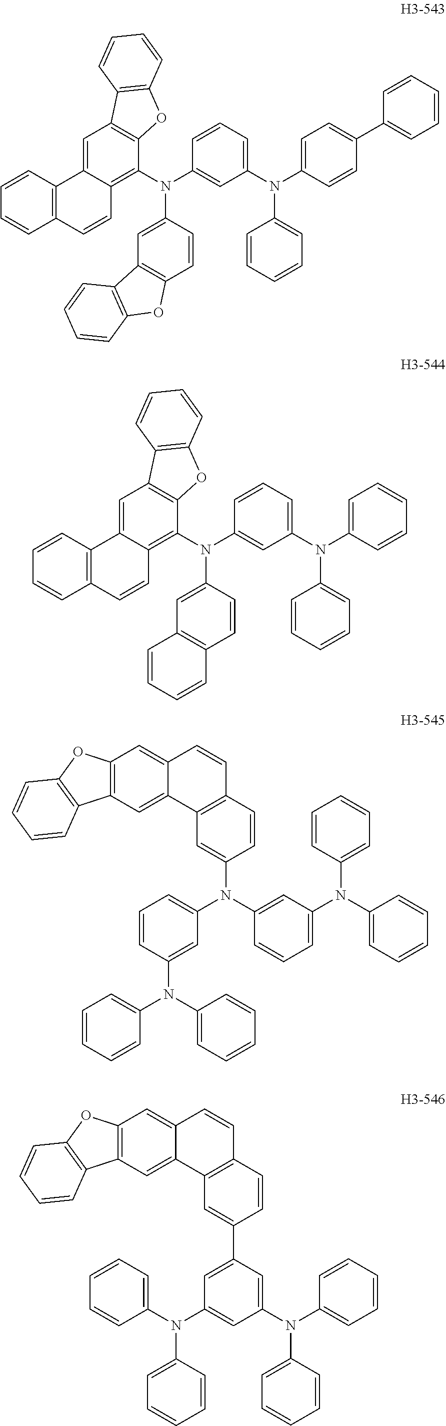 [SMILES: c1ccc(-c2ccc(N(c3ccccc3)c3cccc(N(c4ccc5oc6ccccc6c5c4)c4c5ccc6ccccc6c5cc5c4oc4ccccc45)c3)cc2)cc1 ; c1ccc(N(c2ccccc2)c2cccc(N(c3ccc4ccccc4c3)c3c4ccc5ccccc5c4cc4c3oc3ccccc34)c2)cc1 ; c1ccc(N(c2ccccc2)c2cccc(N(c3cccc(N(c4ccccc4)c4ccccc4)c3)c3ccc4ccc5cc6oc7ccccc7c6cc5c4c3)c2)cc1 ; c1ccc(N(c2ccccc2)c2cc(-c3ccc4ccc5cc6oc7ccccc7c6cc5c4c3)cc(N(c3ccccc3)c3ccccc3)c2)cc1]

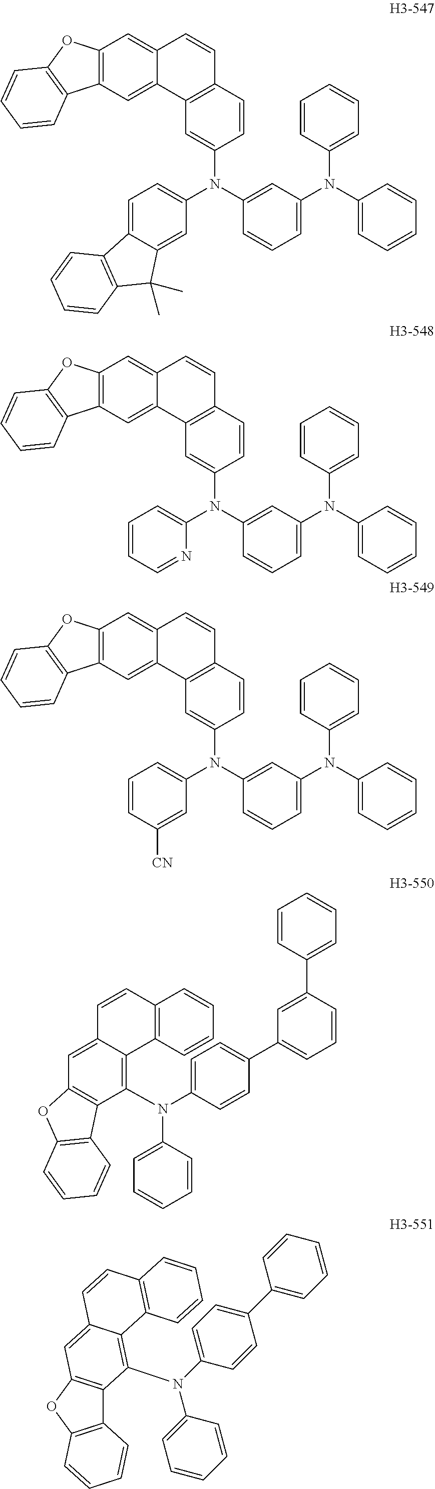

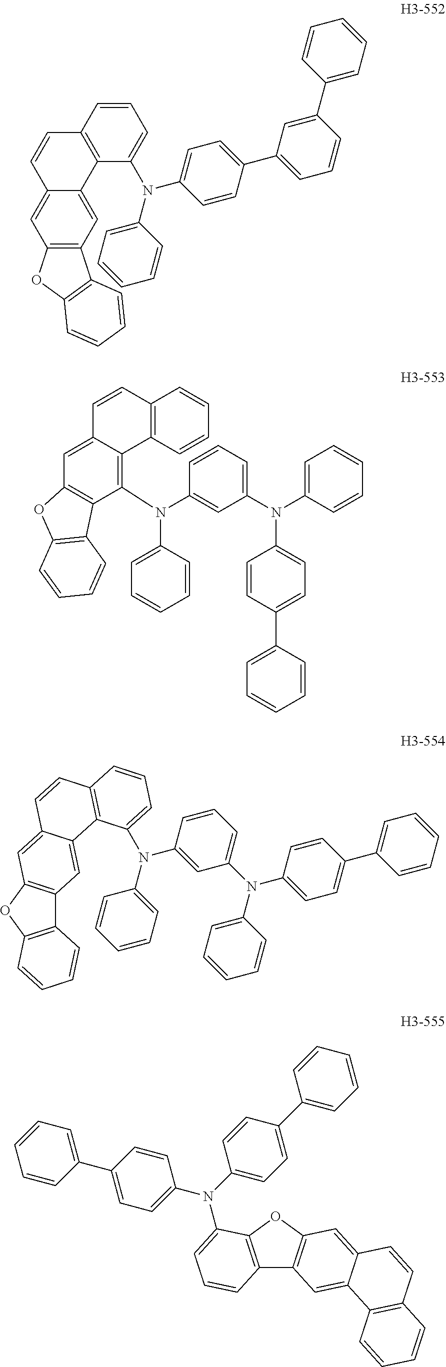

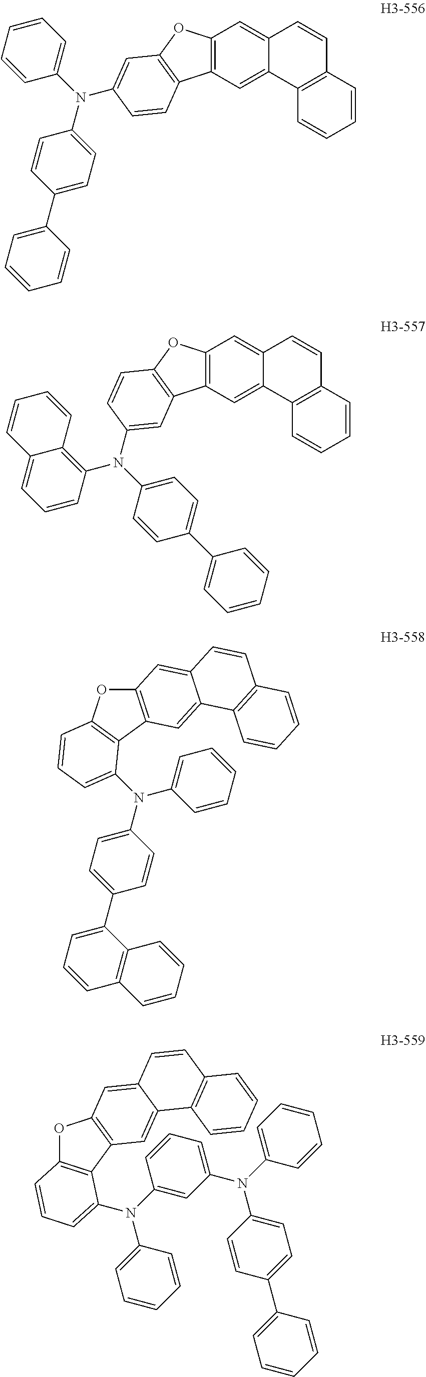

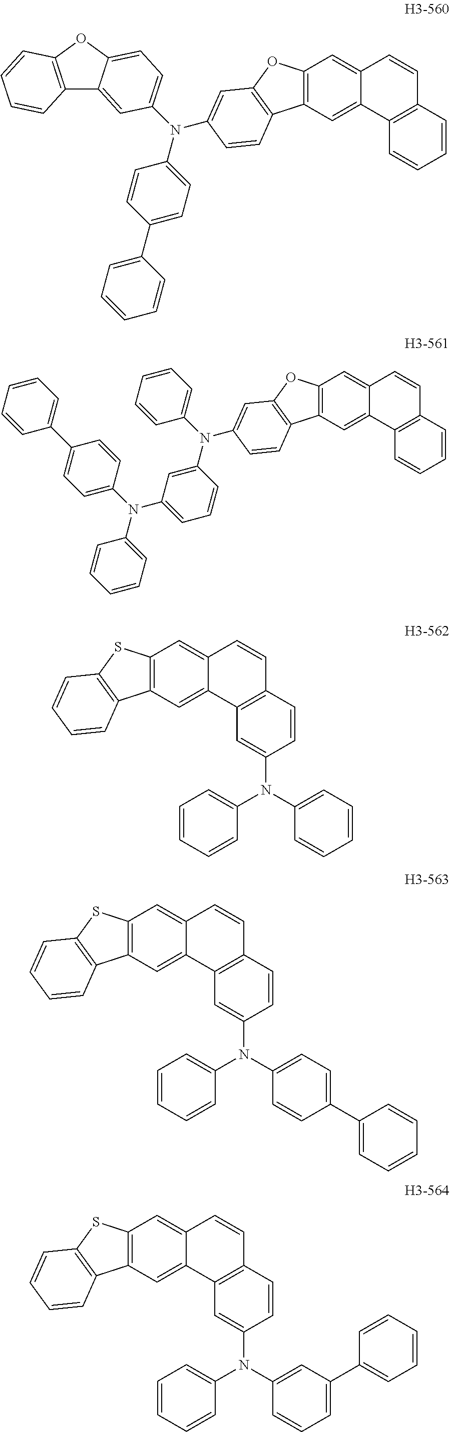 [SMILES: c1ccc(-c2ccc(N(c3ccc4c(c3)oc3cc5ccc6ccccc6c5cc34)c3ccc4oc5ccccc5c4c3)cc2)cc1 ; c1ccc(-c2ccc(N(c3ccccc3)c3cccc(N(c4ccccc4)c4ccc5c(c4)oc4cc6ccc7ccccc7c6cc45)c3)cc2)cc1 ; c1ccc(N(c2ccccc2)c2ccc3ccc4cc5sc6ccccc6c5cc4c3c2)cc1 ; c1ccc(-c2ccc(N(c3ccccc3)c3ccc4ccc5cc6sc7ccccc7c6cc5c4c3)cc2)cc1 ; c1ccc(-c2cccc(N(c3ccccc3)c3ccc4ccc5cc6sc7ccccc7c6cc5c4c3)c2)cc1]

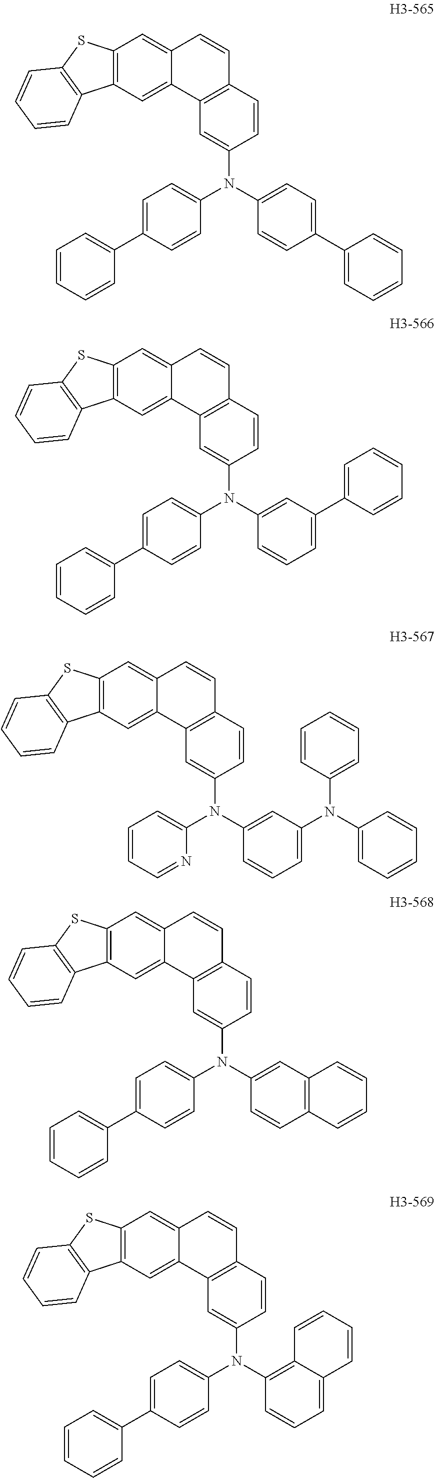

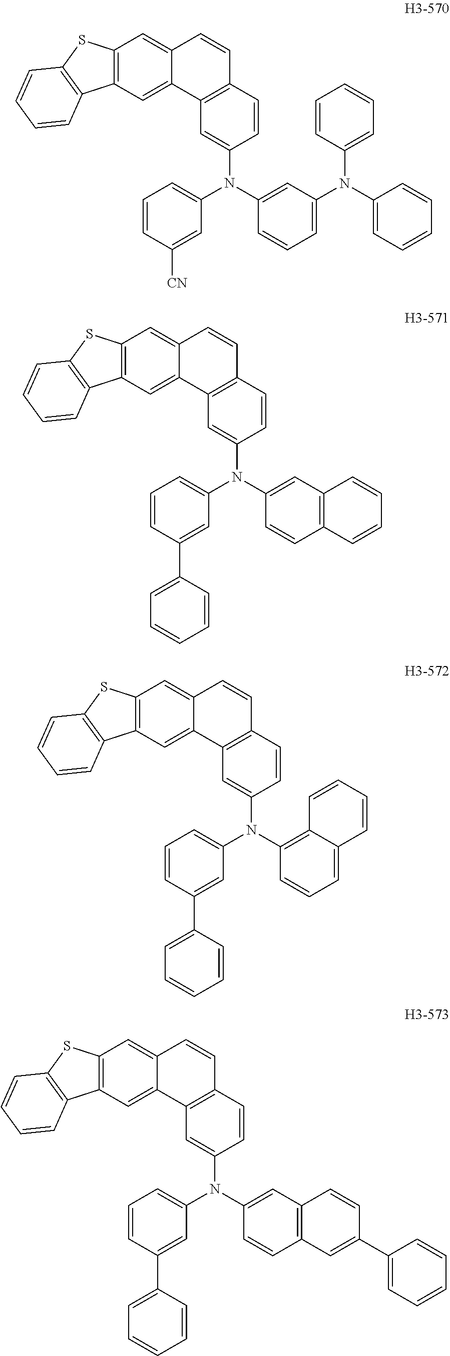

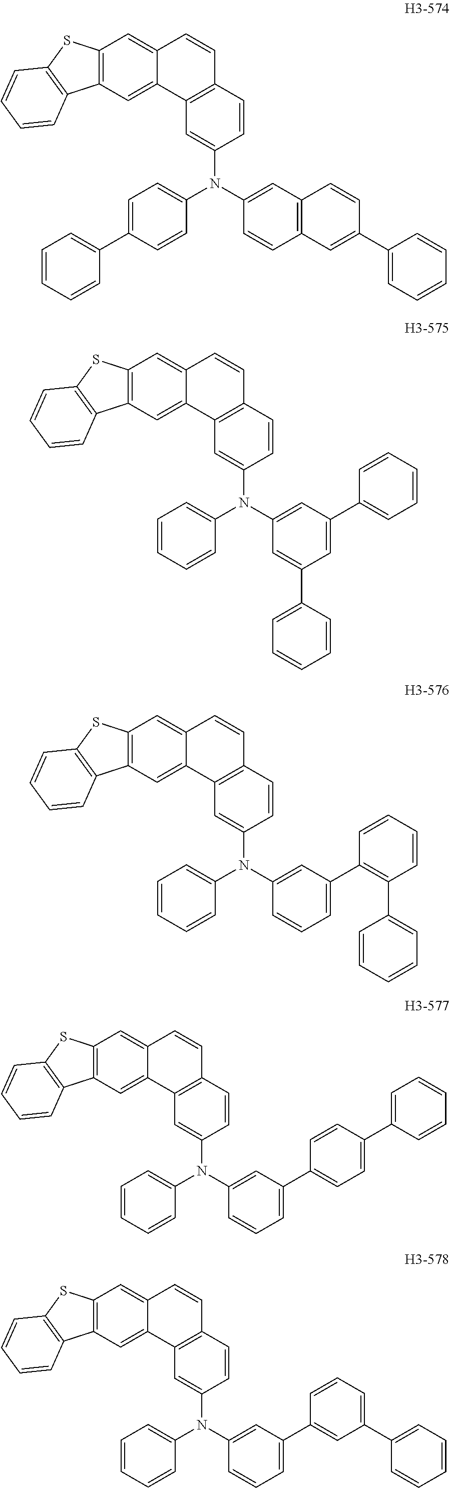 [SMILES: c1ccc(-c2ccc(N(c3ccc4cc(-c5ccccc5)ccc4c3)c3ccc4ccc5cc6sc7ccccc7c6cc5c4c3)cc2)cc1 ; c1ccc(-c2cc(-c3ccccc3)cc(N(c3ccccc3)c3ccc4ccc5cc6sc7ccccc7c6cc5c4c3)c2)cc1 ; c1ccc(-c2ccccc2-c2cccc(N(c3ccccc3)c3ccc4ccc5cc6sc7ccccc7c6cc5c4c3)c2)cc1 ; c1ccc(-c2ccc(-c3cccc(N(c4ccccc4)c4ccc5ccc6cc7sc8ccccc8c7cc6c5c4)c3)cc2)cc1 ; c1ccc(-c2cccc(-c3cccc(N(c4ccccc4)c4ccc5ccc6cc7sc8ccccc8c7cc6c5c4)c3)c2)cc1]

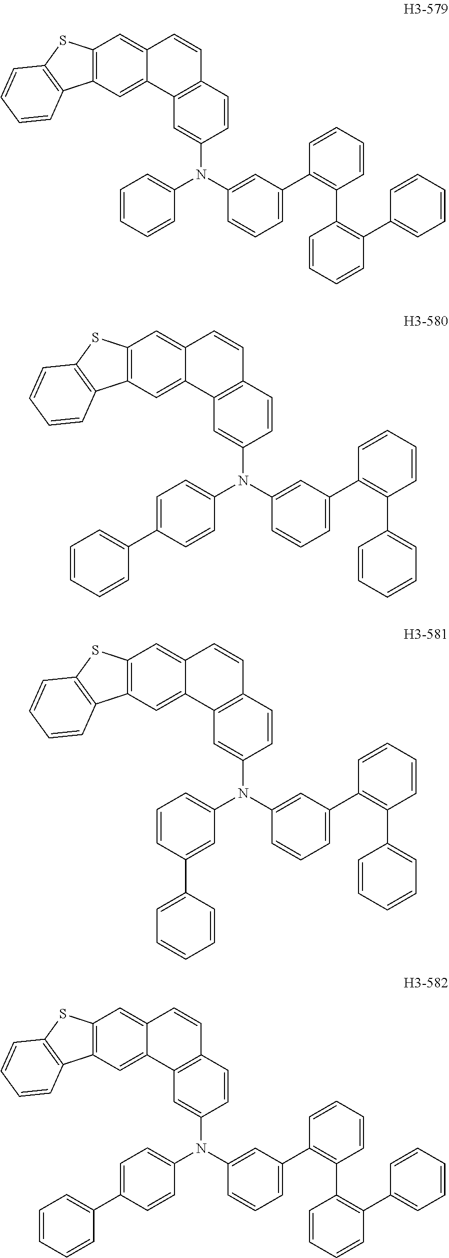 [SMILES: c1ccc(-c2ccccc2-c2ccccc2-c2cccc(N(c3ccccc3)c3ccc4ccc5cc6sc7ccccc7c6cc5c4c3)c2)cc1 ; c1ccc(-c2ccc(N(c3cccc(-c4ccccc4-c4ccccc4)c3)c3ccc4ccc5cc6sc7ccccc7c6cc5c4c3)cc2)cc1 ; c1ccc(-c2cccc(N(c3cccc(-c4ccccc4-c4ccccc4)c3)c3ccc4ccc5cc6sc7ccccc7c6cc5c4c3)c2)cc1 ; c1ccc(-c2ccc(N(c3cccc(-c4ccccc4-c4ccccc4-c4ccccc4)c3)c3ccc4ccc5cc6sc7ccccc7c6cc5c4c3)cc2)cc1]

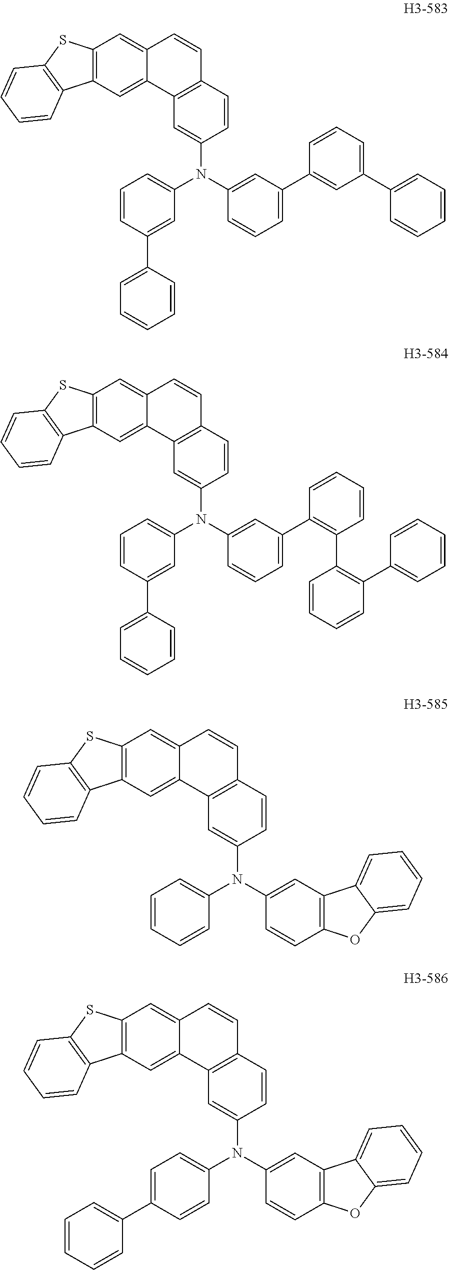 [SMILES: c1ccc(-c2cccc(-c3cccc(N(c4cccc(-c5ccccc5)c4)c4ccc5ccc6cc7sc8ccccc8c7cc6c5c4)c3)c2)cc1 ; c1ccc(-c2cccc(N(c3cccc(-c4ccccc4-c4ccccc4-c4ccccc4)c3)c3ccc4ccc5cc6sc7ccccc7c6cc5c4c3)c2)cc1 ; c1ccc(N(c2ccc3ccc4cc5sc6ccccc6c5cc4c3c2)c2ccc3oc4ccccc4c3c2)cc1 ; c1ccc(-c2ccc(N(c3ccc4ccc5cc6sc7ccccc7c6cc5c4c3)c3ccc4oc5ccccc5c4c3)cc2)cc1]

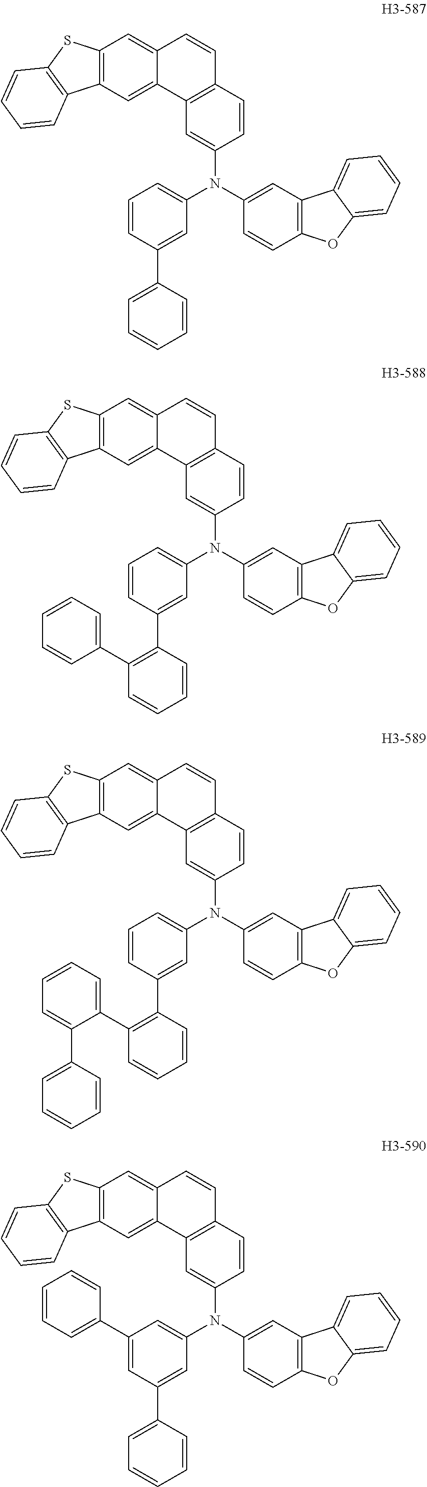 [SMILES: c1ccc(-c2cccc(N(c3ccc4ccc5cc6sc7ccccc7c6cc5c4c3)c3ccc4oc5ccccc5c4c3)c2)cc1 ; c1ccc(-c2ccccc2-c2cccc(N(c3ccc4ccc5cc6sc7ccccc7c6cc5c4c3)c3ccc4oc5ccccc5c4c3)c2)cc1 ; c1ccc(-c2ccccc2-c2ccccc2-c2cccc(N(c3ccc4ccc5cc6sc7ccccc7c6cc5c4c3)c3ccc4oc5ccccc5c4c3)c2)cc1 ; c1ccc(-c2cc(-c3ccccc3)cc(N(c3ccc4ccc5cc6sc7ccccc7c6cc5c4c3)c3ccc4oc5ccccc5c4c3)c2)cc1]

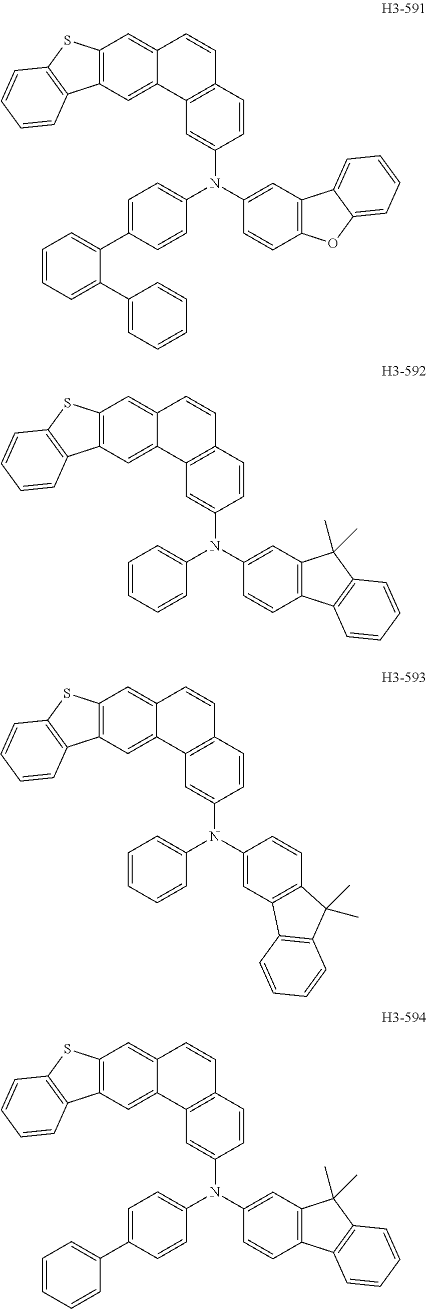 [SMILES: c1ccc(-c2ccccc2-c2ccc(N(c3ccc4ccc5cc6sc7ccccc7c6cc5c4c3)c3ccc4oc5ccccc5c4c3)cc2)cc1 ; CC1(C)c2ccccc2-c2ccc(N(c3ccccc3)c3ccc4ccc5cc6sc7ccccc7c6cc5c4c3)cc21 ; CC1(C)c2ccccc2-c2cc(N(c3ccccc3)c3ccc4ccc5cc6sc7ccccc7c6cc5c4c3)ccc21 ; CC1(C)c2ccccc2-c2ccc(N(c3ccc(-c4ccccc4)cc3)c3ccc4ccc5cc6sc7ccccc7c6cc5c4c3)cc21]

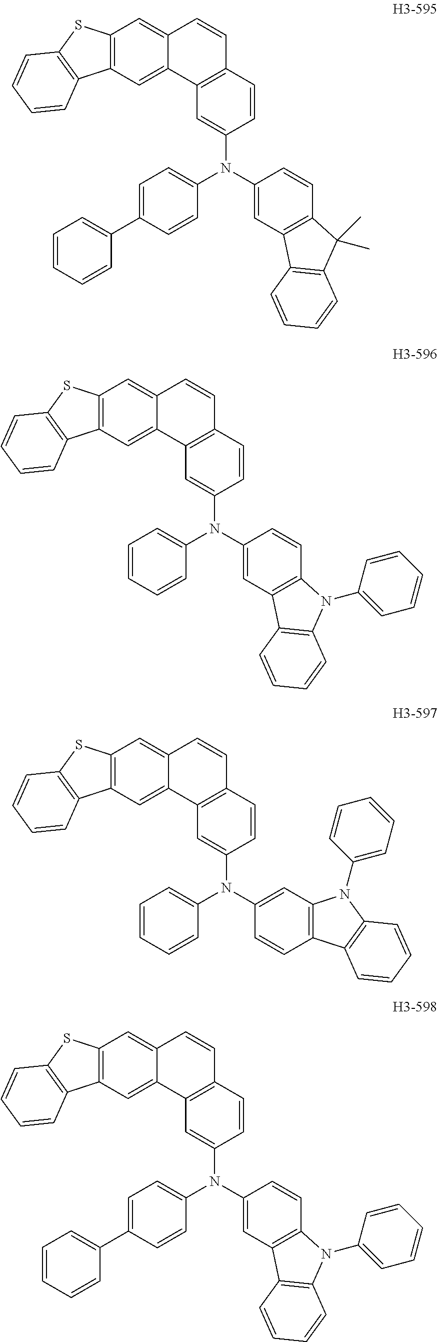 [SMILES: CC1(C)c2ccccc2-c2cc(N(c3ccc(-c4ccccc4)cc3)c3ccc4ccc5cc6sc7ccccc7c6cc5c4c3)ccc21 ; c1ccc(N(c2ccc3ccc4cc5sc6ccccc6c5cc4c3c2)c2ccc3c(c2)c2ccccc2n3-c2ccccc2)cc1 ; c1ccc(N(c2ccc3ccc4cc5sc6ccccc6c5cc4c3c2)c2ccc3c4ccccc4n(-c4ccccc4)c3c2)cc1 ; c1ccc(-c2ccc(N(c3ccc4ccc5cc6sc7ccccc7c6cc5c4c3)c3ccc4c(c3)c3ccccc3n4-c3ccccc3)cc2)cc1]

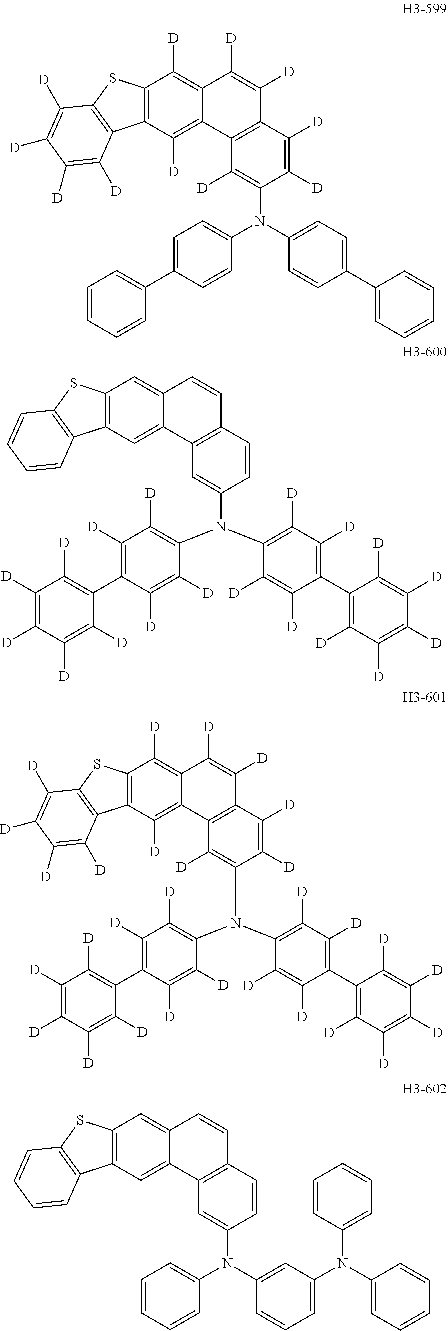

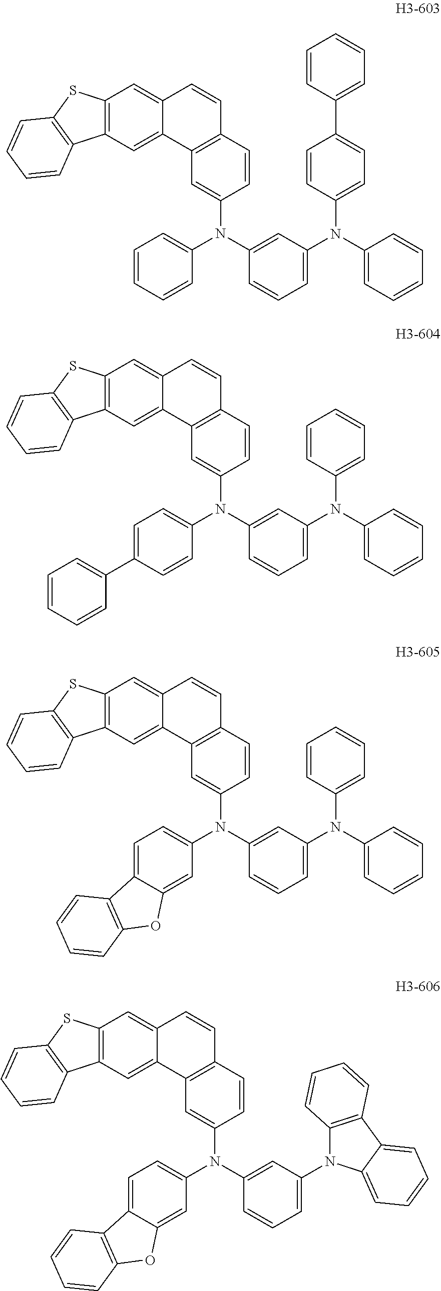 [SMILES: c1ccc(-c2ccc(N(c3ccccc3)c3cccc(N(c4ccccc4)c4ccc5ccc6cc7sc8ccccc8c7cc6c5c4)c3)cc2)cc1 ; c1ccc(-c2ccc(N(c3cccc(N(c4ccccc4)c4ccccc4)c3)c3ccc4ccc5cc6sc7ccccc7c6cc5c4c3)cc2)cc1 ; c1ccc(N(c2ccccc2)c2cccc(N(c3ccc4c(c3)oc3ccccc34)c3ccc4ccc5cc6sc7ccccc7c6cc5c4c3)c2)cc1 ; c1cc(N(c2ccc3c(c2)oc2ccccc23)c2ccc3ccc4cc5sc6ccccc6c5cc4c3c2)cc(-n2c3ccccc3c3ccccc32)c1]

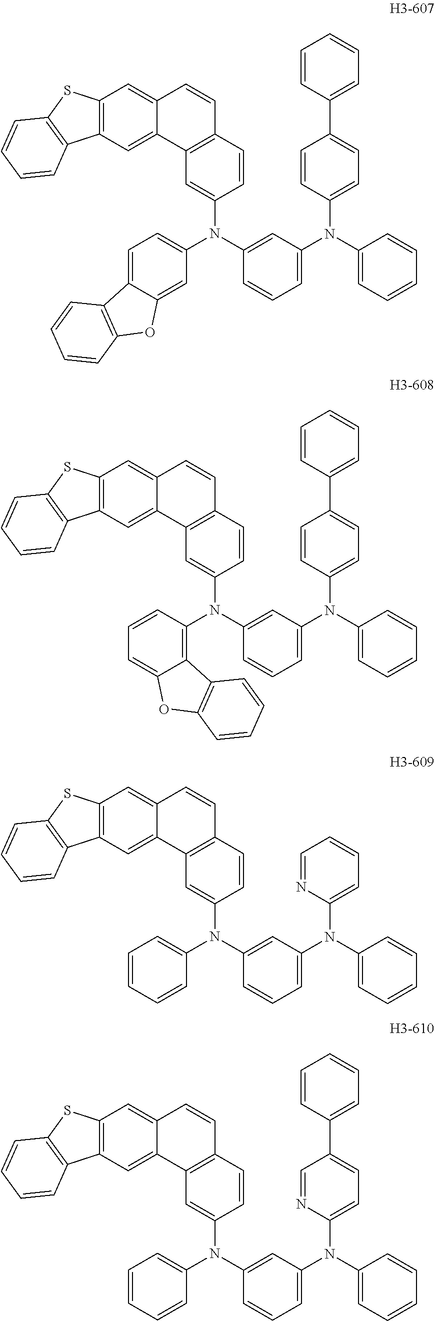 [SMILES: c1ccc(-c2ccc(N(c3ccccc3)c3cccc(N(c4ccc5c(c4)oc4ccccc45)c4ccc5ccc6cc7sc8ccccc8c7cc6c5c4)c3)cc2)cc1 ; c1ccc(-c2ccc(N(c3ccccc3)c3cccc(N(c4ccc5ccc6cc7sc8ccccc8c7cc6c5c4)c4cccc5oc6ccccc6c45)c3)cc2)cc1 ; c1ccc(N(c2cccc(N(c3ccccc3)c3ccccn3)c2)c2ccc3ccc4cc5sc6ccccc6c5cc4c3c2)cc1 ; c1ccc(-c2ccc(N(c3ccccc3)c3cccc(N(c4ccccc4)c4ccc5ccc6cc7sc8ccccc8c7cc6c5c4)c3)nc2)cc1]

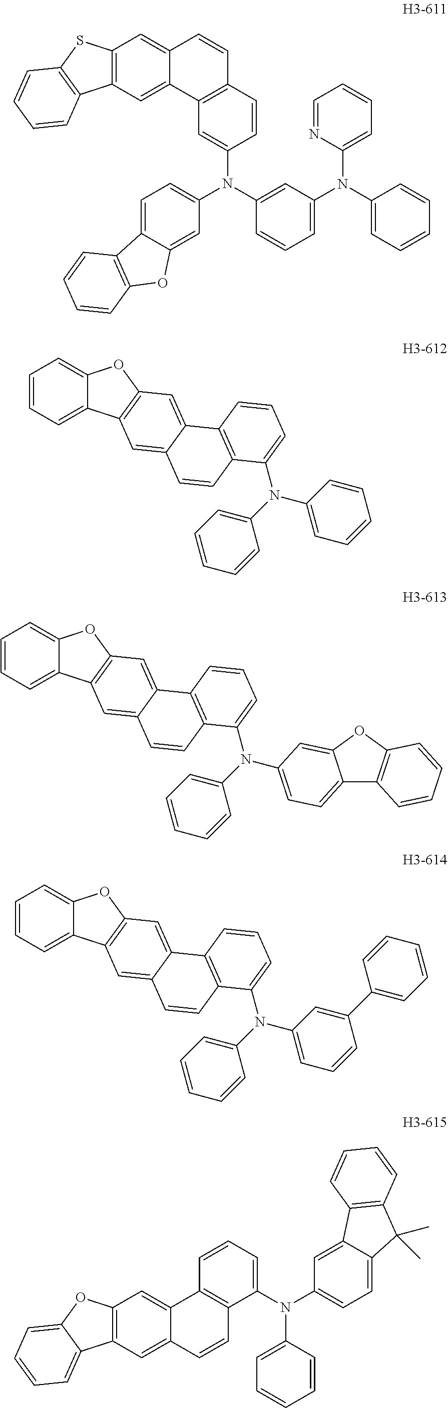 [SMILES: c1ccc(N(c2cccc(N(c3ccc4c(c3)oc3ccccc34)c3ccc4ccc5cc6sc7ccccc7c6cc5c4c3)c2)c2ccccn2)cc1 ; c1ccc(N(c2ccccc2)c2cccc3c2ccc2cc4c(cc23)oc2ccccc24)cc1 ; c1ccc(N(c2ccc3c(c2)oc2ccccc23)c2cccc3c2ccc2cc4c(cc23)oc2ccccc24)cc1 ; c1ccc(-c2cccc(N(c3ccccc3)c3cccc4c3ccc3cc5c(cc34)oc3ccccc35)c2)cc1 ; CC1(C)c2ccccc2-c2cc(N(c3ccccc3)c3cccc4c3ccc3cc5c(cc34)oc3ccccc35)ccc21]

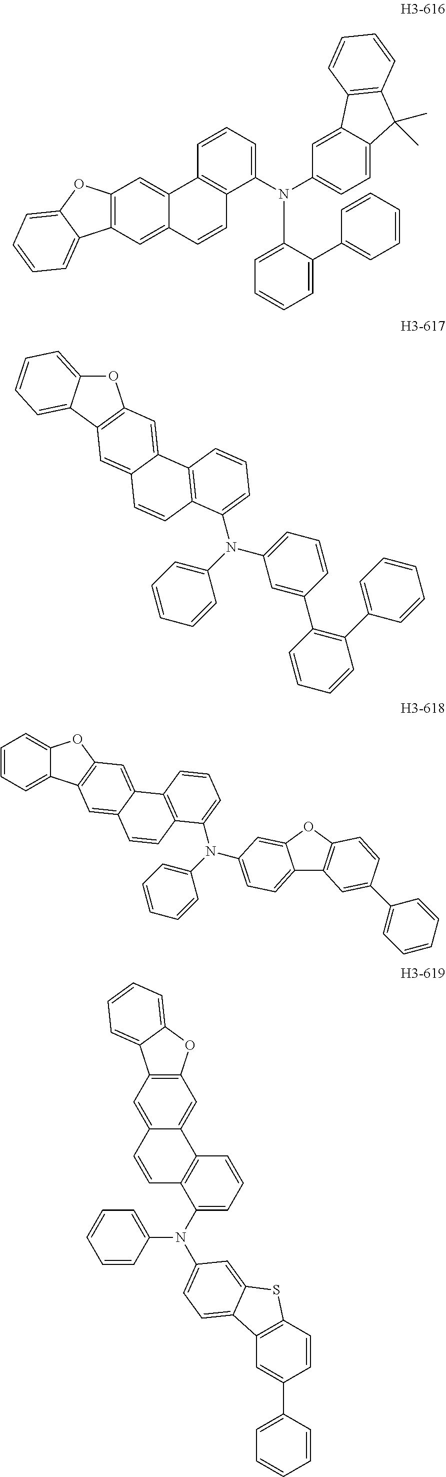 [SMILES: CC1(C)c2ccccc2-c2cc(N(c3ccccc3-c3ccccc3)c3cccc4c3ccc3cc5c(cc34)oc3ccccc35)ccc21 ; c1ccc(-c2ccccc2-c2cccc(N(c3ccccc3)c3cccc4c3ccc3cc5c(cc34)oc3ccccc35)c2)cc1 ; c1ccc(-c2ccc3oc4cc(N(c5ccccc5)c5cccc6c5ccc5cc7c(cc56)oc5ccccc57)ccc4c3c2)cc1 ; c1ccc(-c2ccc3sc4cc(N(c5ccccc5)c5cccc6c5ccc5cc7c(cc56)oc5ccccc57)ccc4c3c2)cc1]

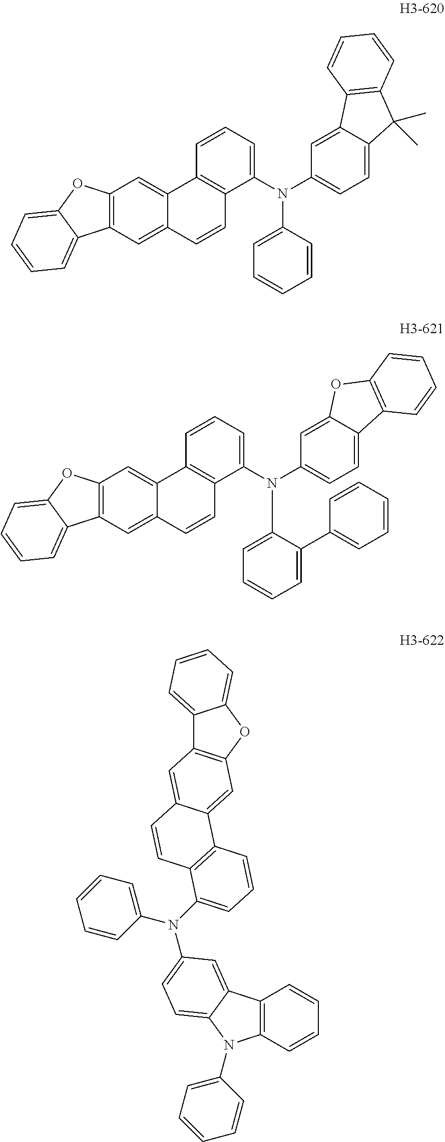 [SMILES: CC1(C)c2ccccc2-c2cc(N(c3ccccc3)c3cccc4c3ccc3cc5c(cc34)oc3ccccc35)ccc21 ; c1ccc(-c2ccccc2N(c2ccc3c(c2)oc2ccccc23)c2cccc3c2ccc2cc4c(cc23)oc2ccccc24)cc1 ; c1ccc(N(c2ccc3c(c2)c2ccccc2n3-c2ccccc2)c2cccc3c2ccc2cc4c(cc23)oc2ccccc24)cc1]

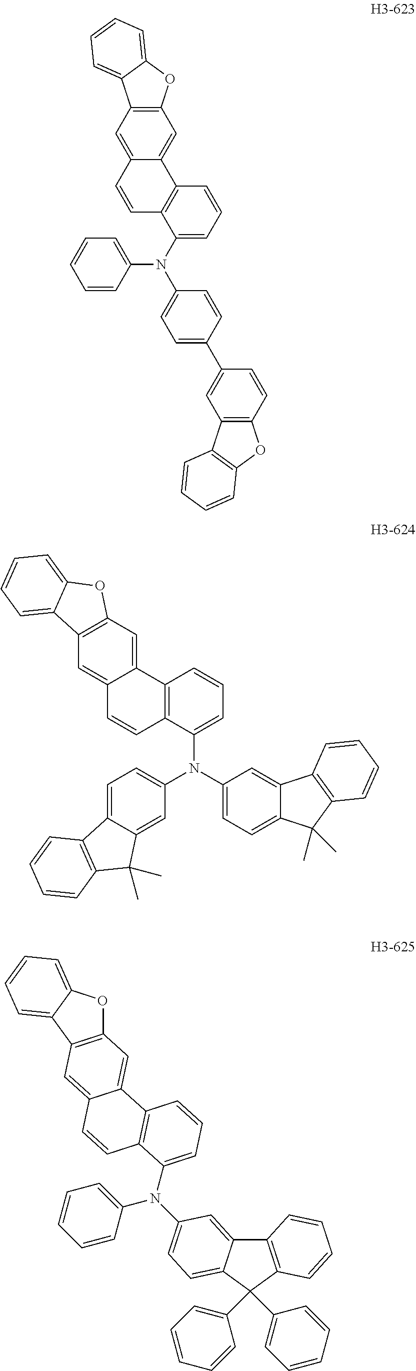 [SMILES: c1ccc(N(c2ccc(-c3ccc4oc5ccccc5c4c3)cc2)c2cccc3c2ccc2cc4c(cc23)oc2ccccc24)cc1 ; CC1(C)c2ccccc2-c2cc(N(c3ccc4c(c3)C(C)(C)c3ccccc3-4)c3cccc4c3ccc3cc5c(cc34)oc3ccccc35)ccc21 ; c1ccc(N(c2ccc3c(c2)-c2ccccc2C3(c2ccccc2)c2ccccc2)c2cccc3c2ccc2cc4c(cc23)oc2ccccc24)cc1]

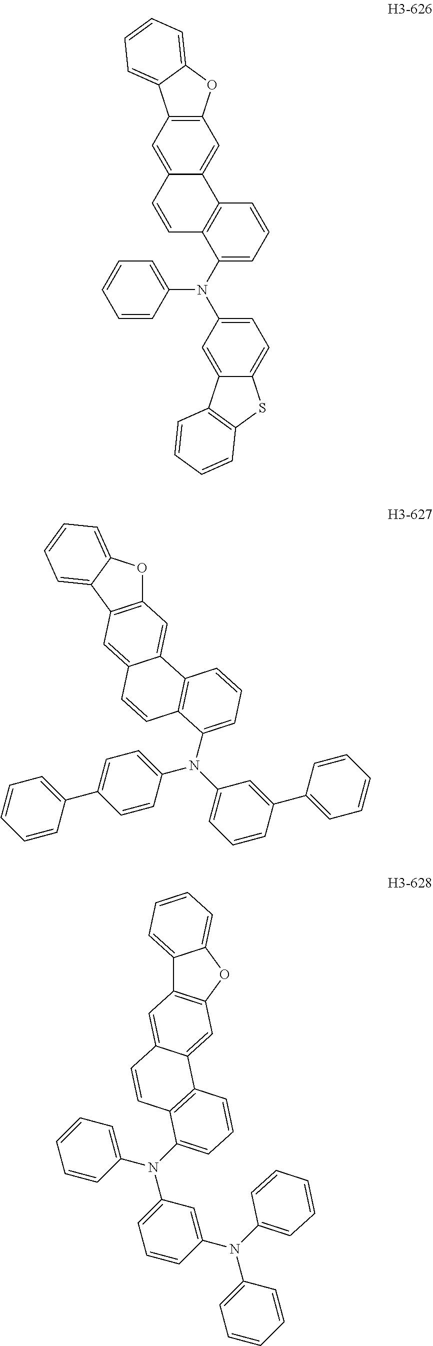 [SMILES: c1ccc(N(c2ccc3sc4ccccc4c3c2)c2cccc3c2ccc2cc4c(cc23)oc2ccccc24)cc1 ; c1ccc(-c2ccc(N(c3cccc(-c4ccccc4)c3)c3cccc4c3ccc3cc5c(cc34)oc3ccccc35)cc2)cc1 ; c1ccc(N(c2ccccc2)c2cccc(N(c3ccccc3)c3cccc4c3ccc3cc5c(cc34)oc3ccccc35)c2)cc1]

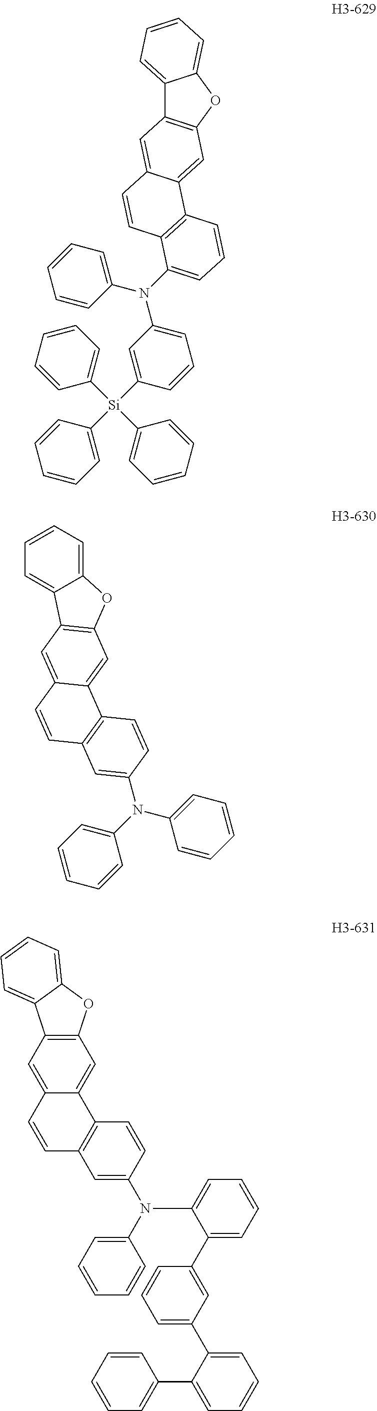 [SMILES: c1ccc(N(c2cccc([Si](c3ccccc3)(c3ccccc3)c3ccccc3)c2)c2cccc3c2ccc2cc4c(cc23)oc2ccccc24)cc1 ; c1ccc(N(c2ccccc2)c2ccc3c(ccc4cc5c(cc43)oc3ccccc35)c2)cc1 ; c1ccc(-c2ccccc2-c2cccc(-c3ccccc3N(c3ccccc3)c3ccc4c(ccc5cc6c(cc54)oc4ccccc46)c3)c2)cc1]

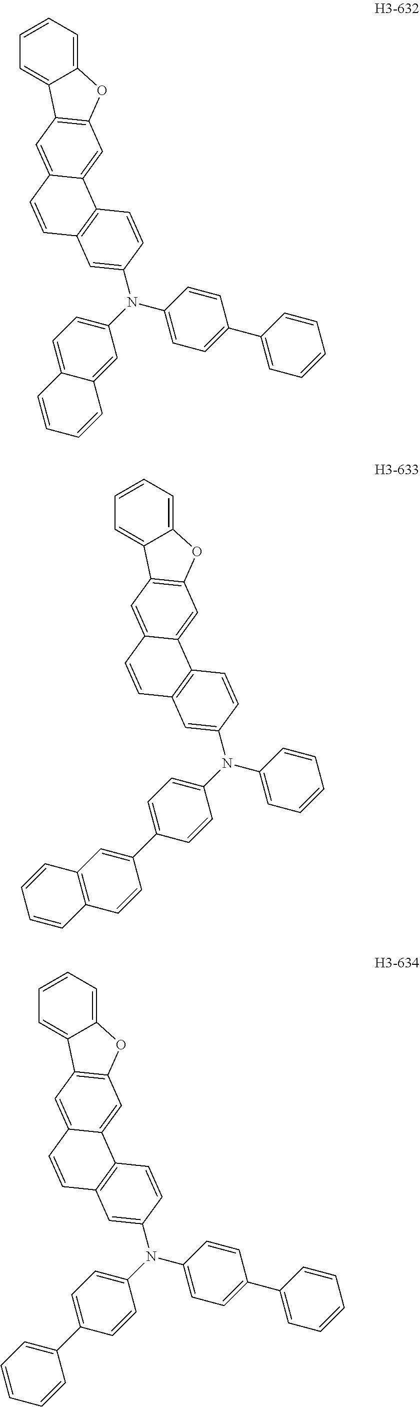

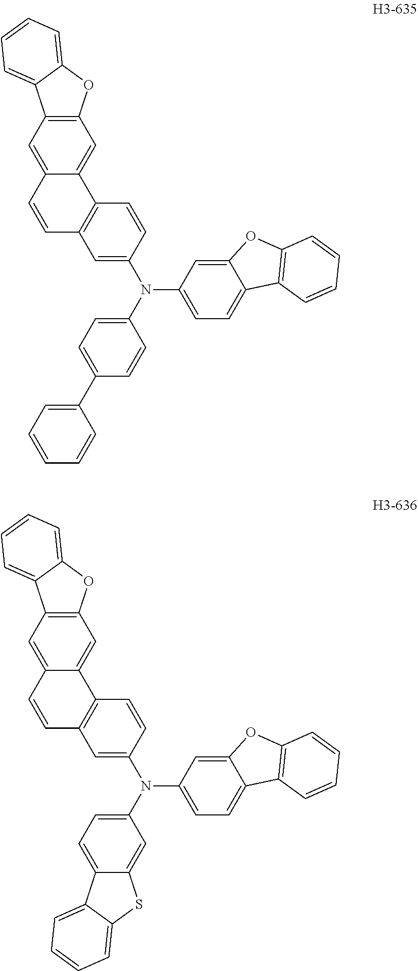 [SMILES: c1ccc(-c2ccc(N(c3ccc4c(ccc5cc6c(cc54)oc4ccccc46)c3)c3ccc4c(c3)oc3ccccc34)cc2)cc1 ; c1ccc2c(c1)oc1cc(N(c3ccc4c(ccc5cc6c(cc54)oc4ccccc46)c3)c3ccc4c(c3)sc3ccccc34)ccc12]

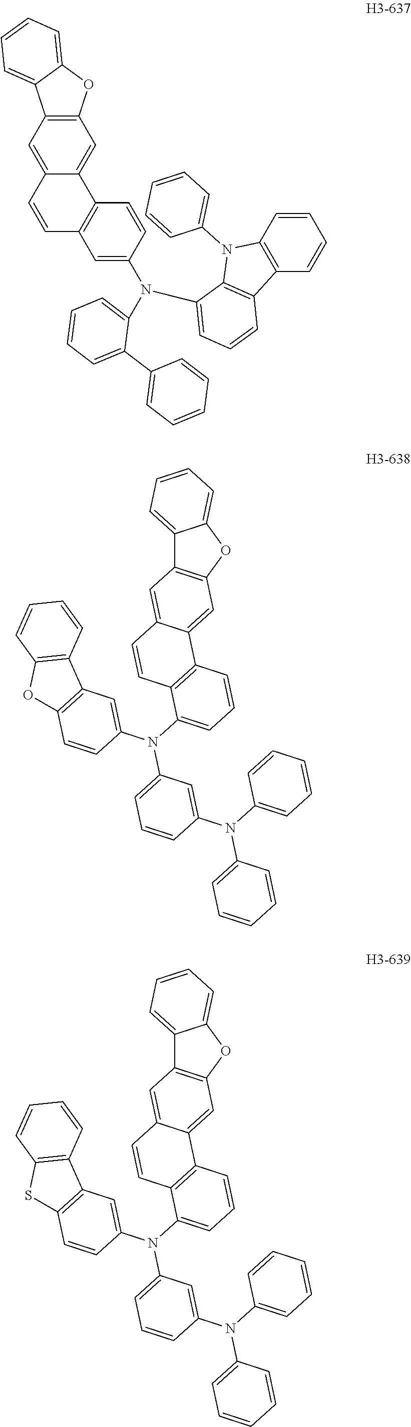

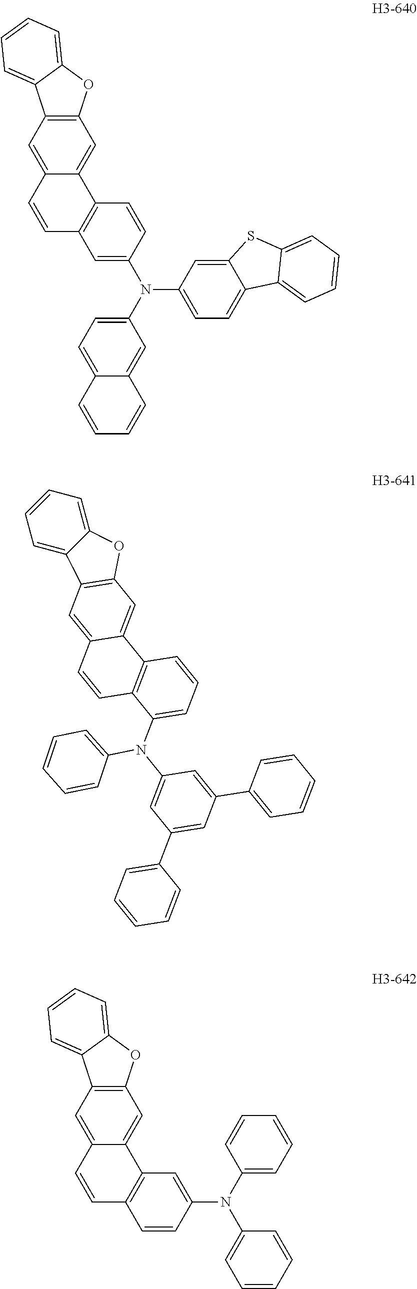 [SMILES: c1ccc2cc(N(c3ccc4c(ccc5cc6c(cc54)oc4ccccc46)c3)c3ccc4c(c3)sc3ccccc34)ccc2c1 ; c1ccc(-c2cc(-c3ccccc3)cc(N(c3ccccc3)c3cccc4c3ccc3cc5c(cc34)oc3ccccc35)c2)cc1 ; c1ccc(N(c2ccccc2)c2ccc3ccc4cc5c(cc4c3c2)oc2ccccc25)cc1]

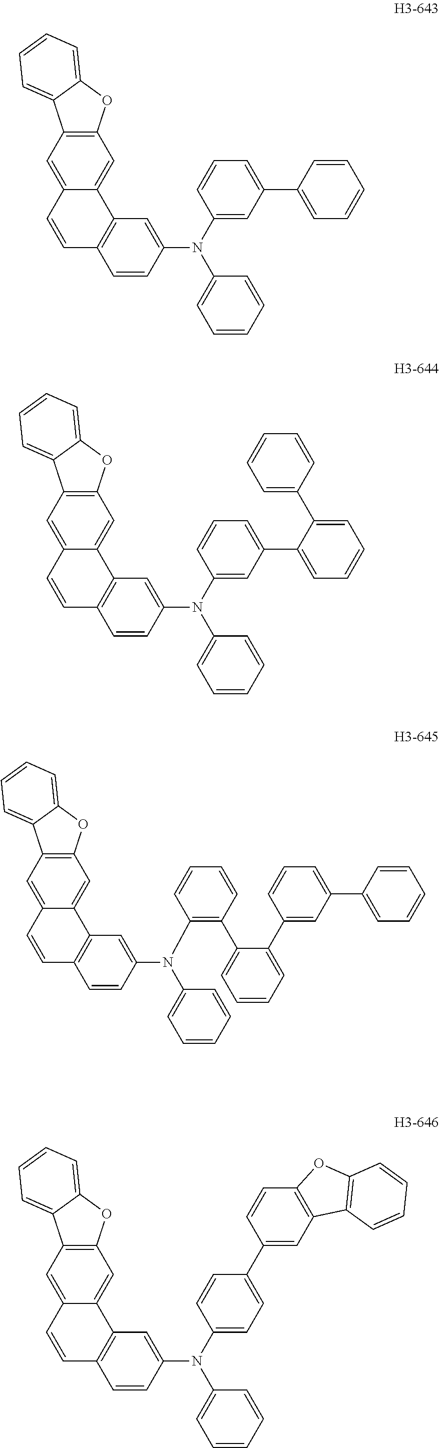 [SMILES: c1ccc(-c2cccc(N(c3ccccc3)c3ccc4ccc5cc6c(cc5c4c3)oc3ccccc36)c2)cc1 ; c1ccc(-c2ccccc2-c2cccc(N(c3ccccc3)c3ccc4ccc5cc6c(cc5c4c3)oc3ccccc36)c2)cc1 ; c1ccc(-c2cccc(-c3ccccc3-c3ccccc3N(c3ccccc3)c3ccc4ccc5cc6c(cc5c4c3)oc3ccccc36)c2)cc1 ; c1ccc(N(c2ccc(-c3ccc4oc5ccccc5c4c3)cc2)c2ccc3ccc4cc5c(cc4c3c2)oc2ccccc25)cc1]

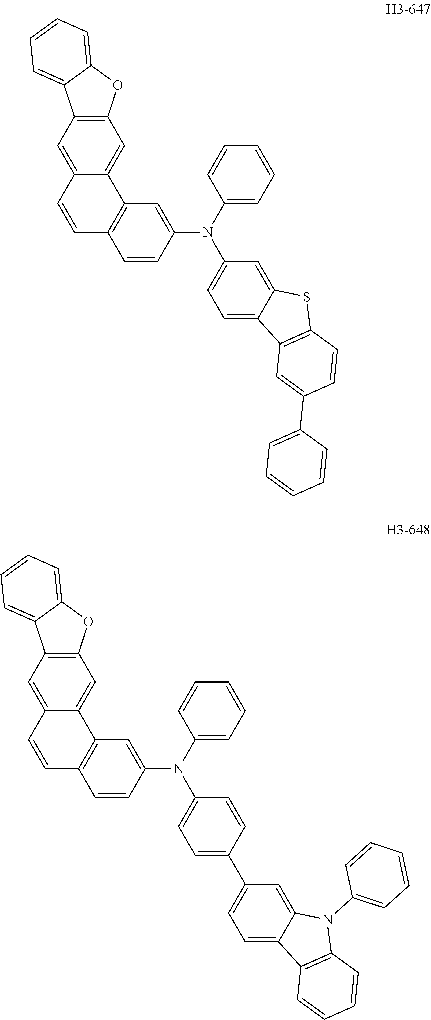 [SMILES: c1ccc(-c2ccc3sc4cc(N(c5ccccc5)c5ccc6ccc7cc8c(cc7c6c5)oc5ccccc58)ccc4c3c2)cc1 ; c1ccc(N(c2ccc(-c3ccc4c5ccccc5n(-c5ccccc5)c4c3)cc2)c2ccc3ccc4cc5c(cc4c3c2)oc2ccccc25)cc1]

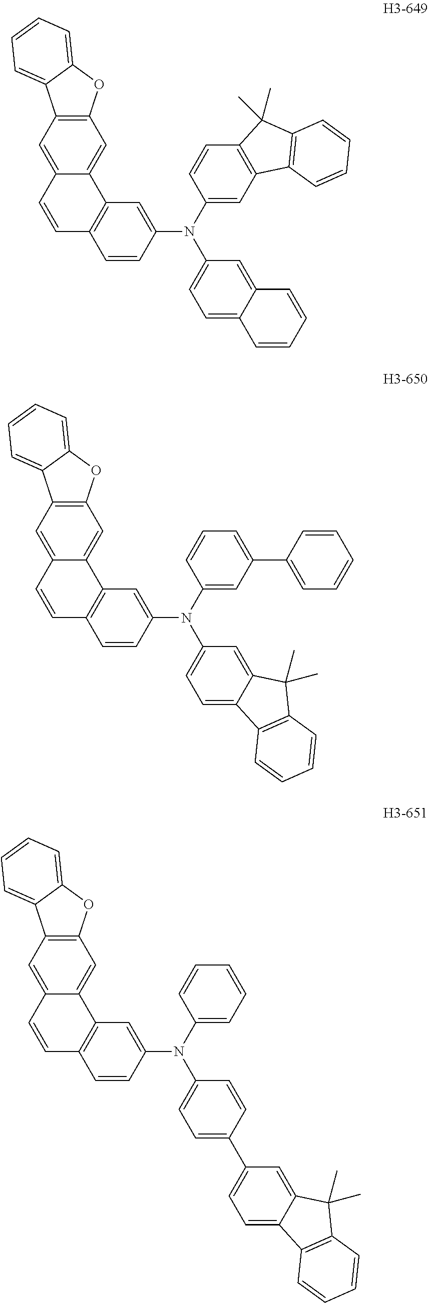 [SMILES: CC1(C)c2ccccc2-c2cc(N(c3ccc4ccccc4c3)c3ccc4ccc5cc6c(cc5c4c3)oc3ccccc36)ccc21 ; CC1(C)c2ccccc2-c2ccc(N(c3cccc(-c4ccccc4)c3)c3ccc4ccc5cc6c(cc5c4c3)oc3ccccc36)cc21 ; CC1(C)c2ccccc2-c2ccc(-c3ccc(N(c4ccccc4)c4ccc5ccc6cc7c(cc6c5c4)oc4ccccc47)cc3)cc21]

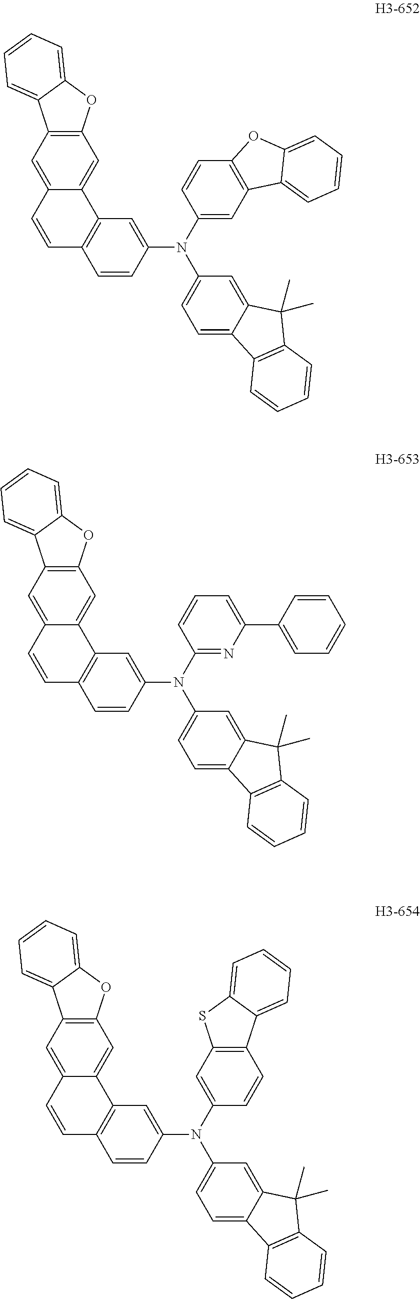 [SMILES: CC1(C)c2ccccc2-c2ccc(N(c3ccc4ccc5cc6c(cc5c4c3)oc3ccccc36)c3ccc4oc5ccccc5c4c3)cc21 ; CC1(C)c2ccccc2-c2ccc(N(c3ccc4ccc5cc6c(cc5c4c3)oc3ccccc36)c3cccc(-c4ccccc4)n3)cc21 ; CC1(C)c2ccccc2-c2ccc(N(c3ccc4c(c3)sc3ccccc34)c3ccc4ccc5cc6c(cc5c4c3)oc3ccccc36)cc21]

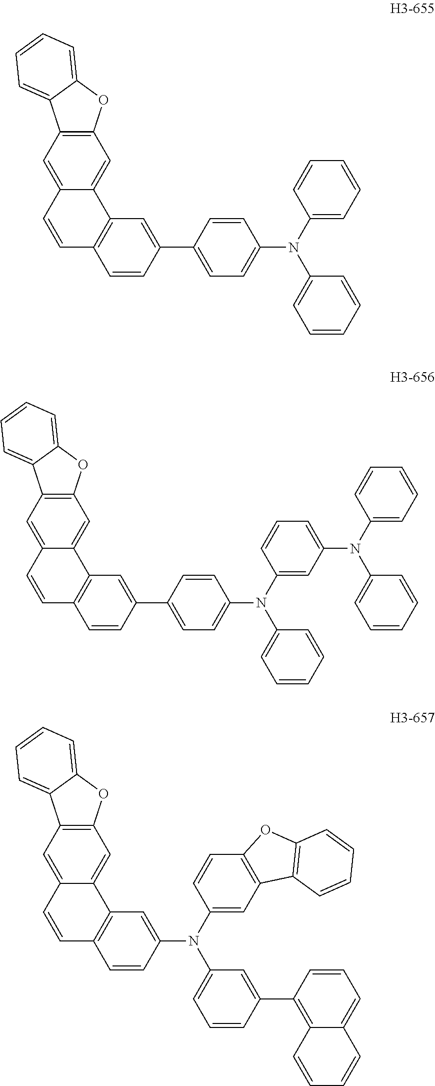 [SMILES: c1ccc(N(c2ccccc2)c2ccc(-c3ccc4ccc5cc6c(cc5c4c3)oc3ccccc36)cc2)cc1 ; c1ccc(N(c2ccccc2)c2cccc(N(c3ccccc3)c3ccc(-c4ccc5ccc6cc7c(cc6c5c4)oc4ccccc47)cc3)c2)cc1 ; c1cc(-c2cccc3ccccc23)cc(N(c2ccc3ccc4cc5c(cc4c3c2)oc2ccccc25)c2ccc3oc4ccccc4c3c2)c1]

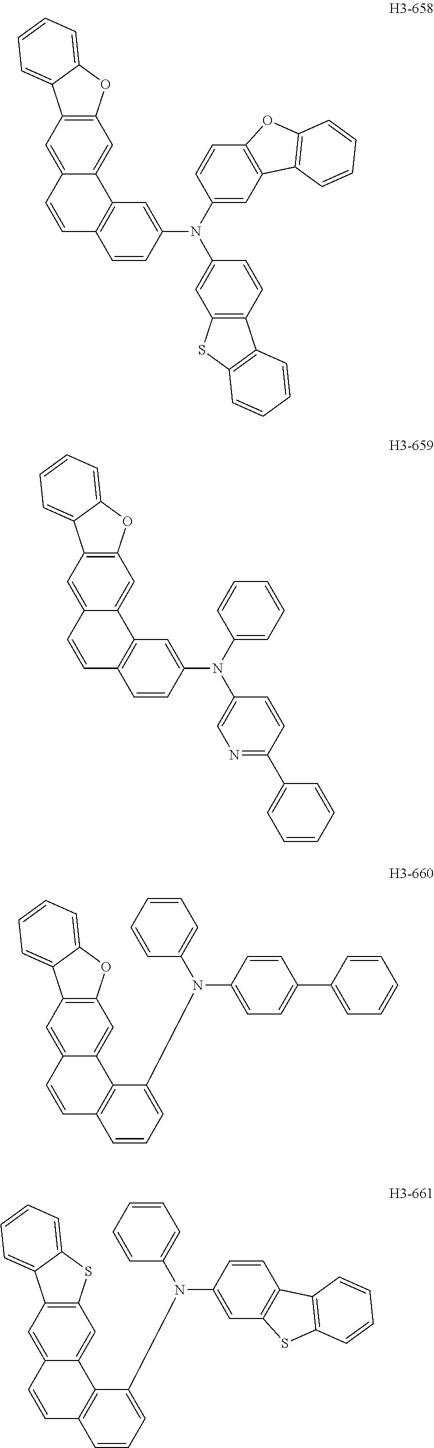 [SMILES: c1ccc2c(c1)oc1ccc(N(c3ccc4c(c3)sc3ccccc34)c3ccc4ccc5cc6c(cc5c4c3)oc3ccccc36)cc12 ; c1ccc(-c2ccc(N(c3ccccc3)c3ccc4ccc5cc6c(cc5c4c3)oc3ccccc36)cn2)cc1 ; c1ccc(-c2ccc(N(c3ccccc3)c3cccc4ccc5cc6c(cc5c34)oc3ccccc36)cc2)cc1 ; c1ccc(N(c2ccc3c(c2)sc2ccccc23)c2cccc3ccc4cc5c(cc4c23)sc2ccccc25)cc1]

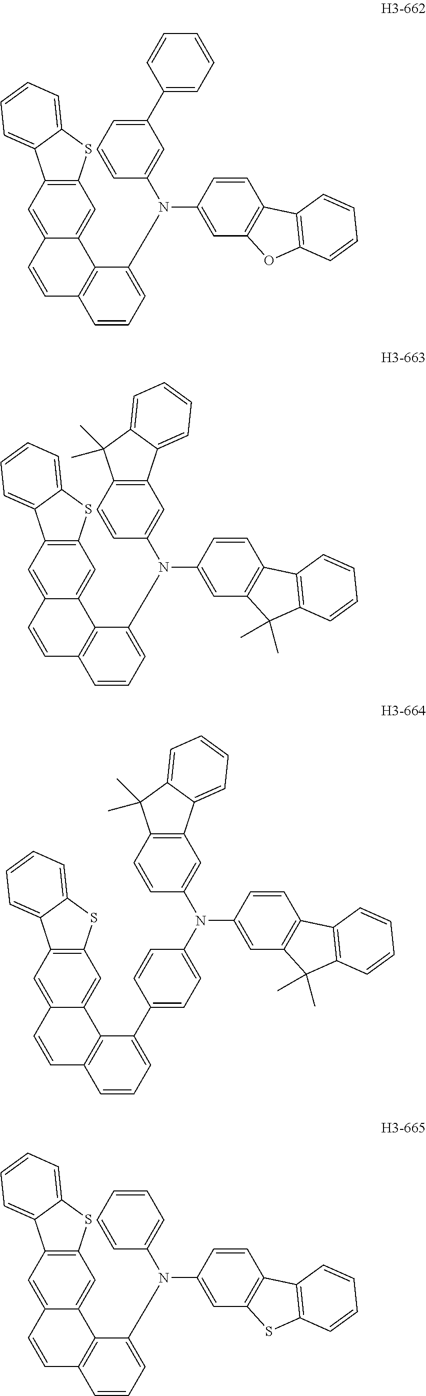 [SMILES: c1ccc(-c2cccc(N(c3ccc4c(c3)oc3ccccc34)c3cccc4ccc5cc6c(cc5c34)sc3ccccc36)c2)cc1 ; CC1(C)c2ccccc2-c2cc(N(c3ccc4c(c3)C(C)(C)c3ccccc3-4)c3cccc4ccc5cc6c(cc5c34)sc3ccccc36)ccc21 ; CC1(C)c2ccccc2-c2cc(N(c3ccc(-c4cccc5ccc6cc7c(cc6c45)sc4ccccc47)cc3)c3ccc4c(c3)C(C)(C)c3ccccc3-4)ccc21 ; c1ccc(N(c2ccc3c(c2)sc2ccccc23)c2cccc3ccc4cc5c(cc4c23)sc2ccccc25)cc1]

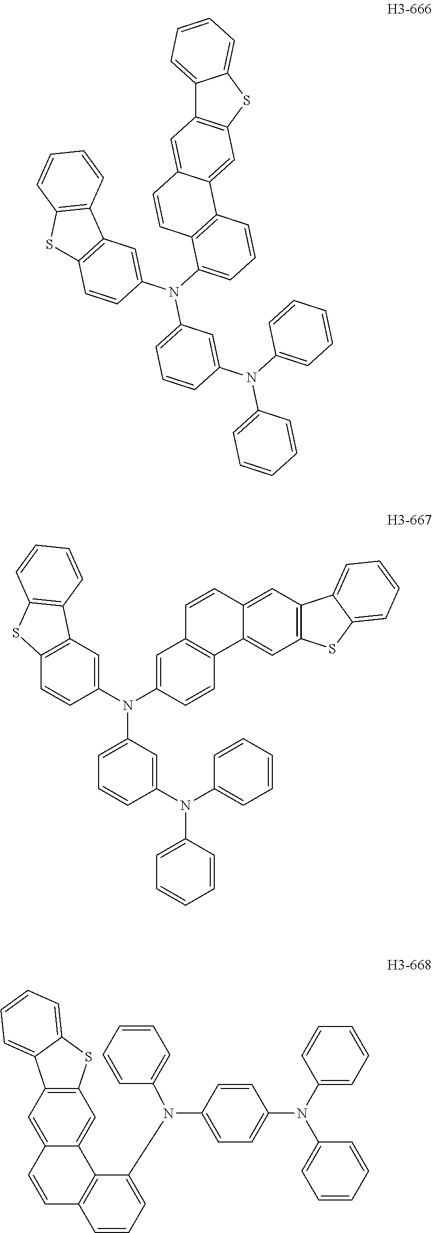 [SMILES: c1ccc(N(c2ccccc2)c2cccc(N(c3ccc4sc5ccccc5c4c3)c3cccc4c3ccc3cc5c(cc34)sc3ccccc35)c2)cc1 ; c1ccc(N(c2ccccc2)c2cccc(N(c3ccc4c(ccc5cc6c(cc54)sc4ccccc46)c3)c3ccc4sc5ccccc5c4c3)c2)cc1 ; c1ccc(N(c2ccccc2)c2ccc(N(c3ccccc3)c3cccc4ccc5cc6c(cc5c34)sc3ccccc36)cc2)cc1]

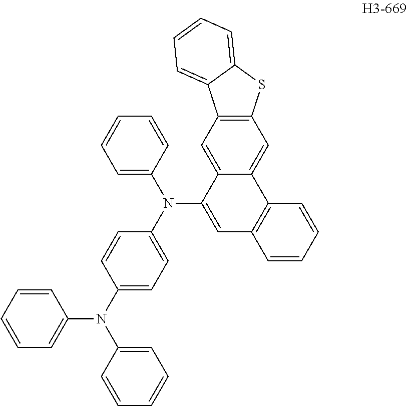 [SMILES: c1ccc(N(c2ccccc2)c2ccc(N(c3ccccc3)c3cc4ccccc4c4cc5sc6ccccc6c5cc34)cc2)cc1]

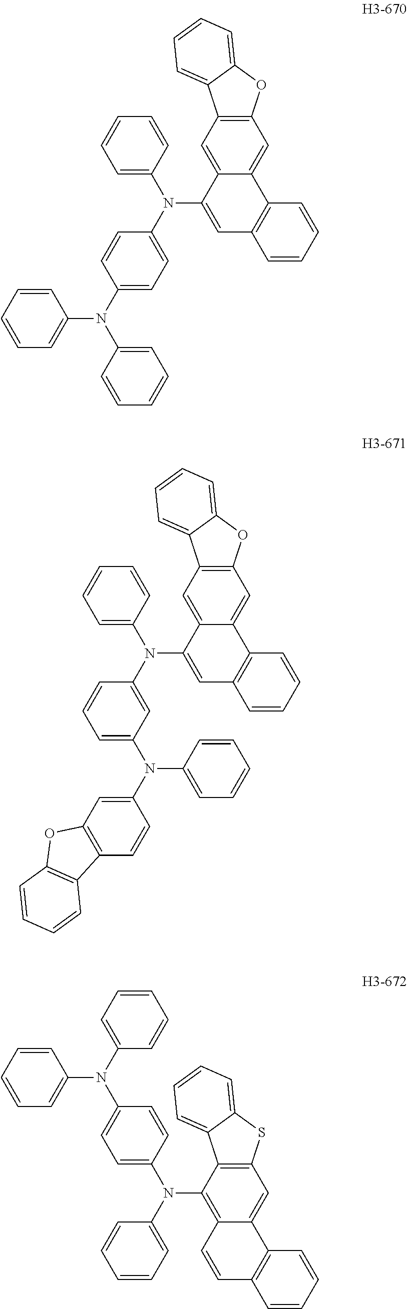 [SMILES: c1ccc(N(c2ccccc2)c2ccc(N(c3ccccc3)c3cc4ccccc4c4cc5oc6ccccc6c5cc34)cc2)cc1 ; c1ccc(N(c2cccc(N(c3ccccc3)c3cc4ccccc4c4cc5oc6ccccc6c5cc34)c2)c2ccc3c(c2)oc2ccccc23)cc1 ; c1ccc(N(c2ccccc2)c2ccc(N(c3ccccc3)c3c4ccc5ccccc5c4cc4sc5ccccc5c34)cc2)cc1]

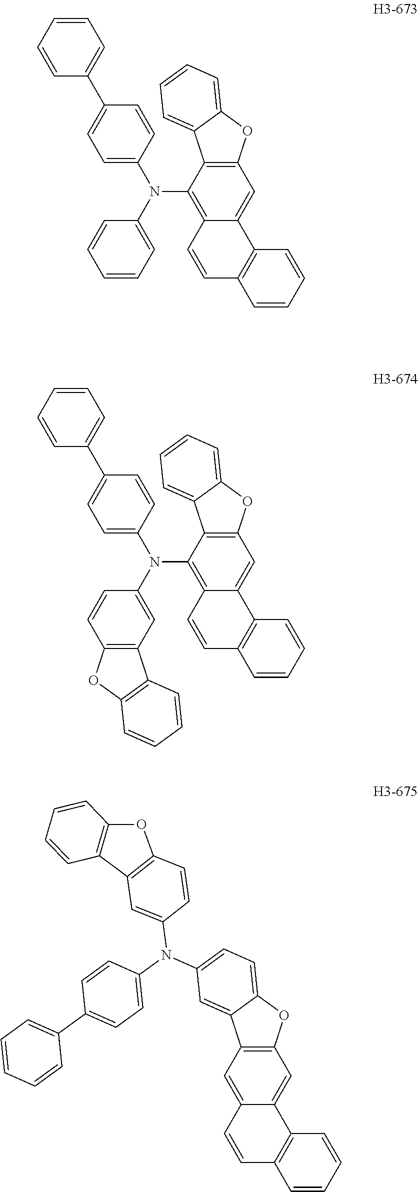 [SMILES: c1ccc(-c2ccc(N(c3ccccc3)c3c4ccc5ccccc5c4cc4oc5ccccc5c34)cc2)cc1 ; c1ccc(-c2ccc(N(c3ccc4oc5ccccc5c4c3)c3c4ccc5ccccc5c4cc4oc5ccccc5c34)cc2)cc1 ; c1ccc(-c2ccc(N(c3ccc4oc5ccccc5c4c3)c3ccc4oc5cc6c(ccc7ccccc76)cc5c4c3)cc2)cc1]

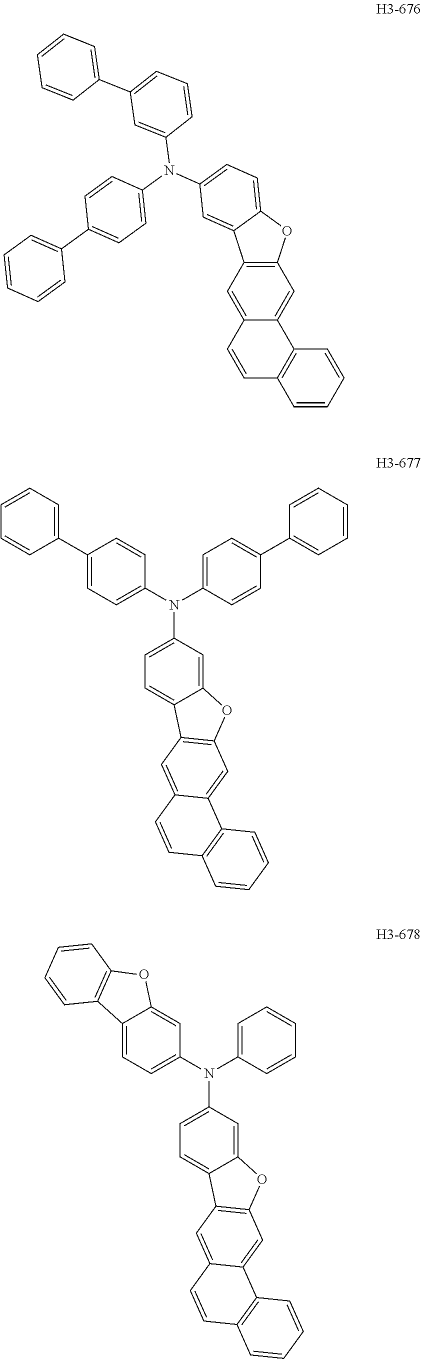 [SMILES: c1ccc(-c2ccc(N(c3cccc(-c4ccccc4)c3)c3ccc4oc5cc6c(ccc7ccccc76)cc5c4c3)cc2)cc1 ; c1ccc(-c2ccc(N(c3ccc(-c4ccccc4)cc3)c3ccc4c(c3)oc3cc5c(ccc6ccccc65)cc34)cc2)cc1 ; c1ccc(N(c2ccc3c(c2)oc2ccccc23)c2ccc3c(c2)oc2cc4c(ccc5ccccc54)cc23)cc1]

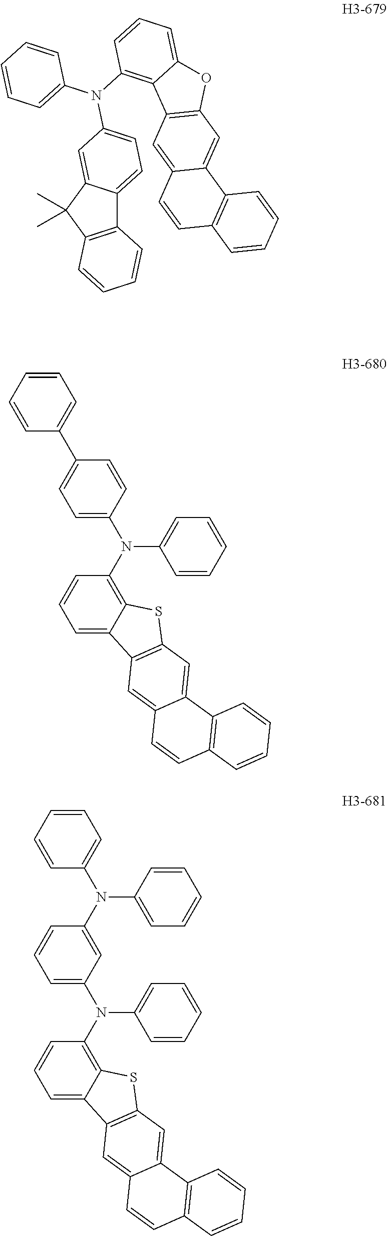 [SMILES: CC1(C)c2ccccc2-c2ccc(N(c3ccccc3)c3cccc4oc5cc6c(ccc7ccccc76)cc5c34)cc21 ; c1ccc(-c2ccc(N(c3ccccc3)c3cccc4c3sc3cc5c(ccc6ccccc65)cc34)cc2)cc1 ; c1ccc(N(c2ccccc2)c2cccc(N(c3ccccc3)c3cccc4c3sc3cc5c(ccc6ccccc65)cc34)c2)cc1]

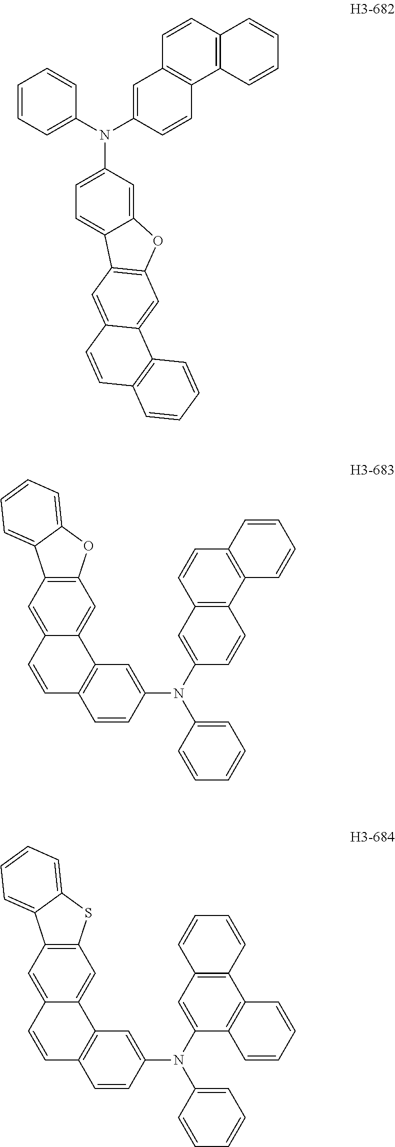 [SMILES: c1ccc(N(c2ccc3c(ccc4ccccc43)c2)c2ccc3c(c2)oc2cc4c(ccc5ccccc54)cc23)cc1 ; c1ccc(N(c2ccc3c(ccc4ccccc43)c2)c2ccc3ccc4cc5c(cc4c3c2)oc2ccccc25)cc1 ; c1ccc(N(c2ccc3ccc4cc5c(cc4c3c2)sc2ccccc25)c2cc3ccccc3c3ccccc23)cc1]

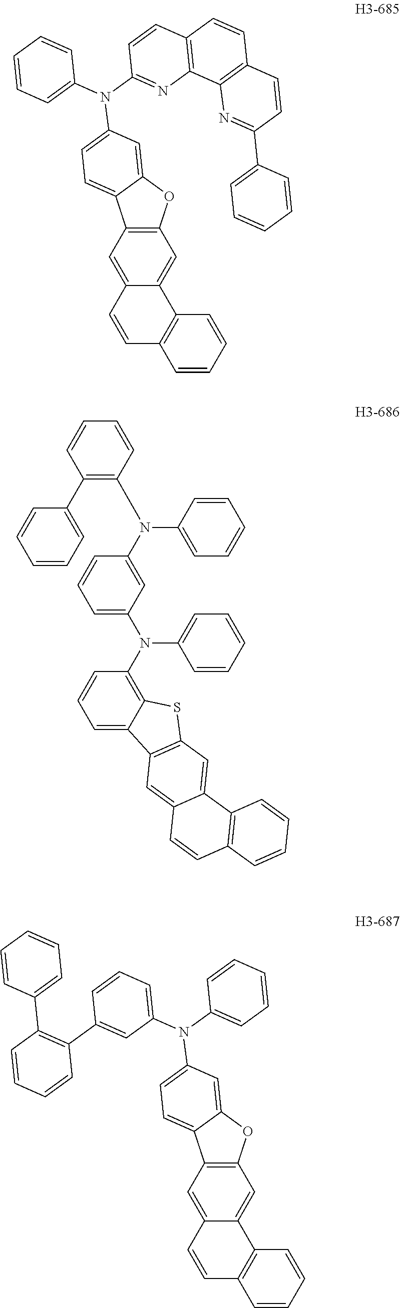 [SMILES: c1ccc(-c2ccc3ccc4ccc(N(c5ccccc5)c5ccc6c(c5)oc5cc7c(ccc8ccccc87)cc56)nc4c3n2)cc1 ; c1ccc(-c2ccccc2N(c2ccccc2)c2cccc(N(c3ccccc3)c3cccc4c3sc3cc5c(ccc6ccccc65)cc34)c2)cc1 ; c1ccc(-c2ccccc2-c2cccc(N(c3ccccc3)c3ccc4c(c3)oc3cc5c(ccc6ccccc65)cc34)c2)cc1]

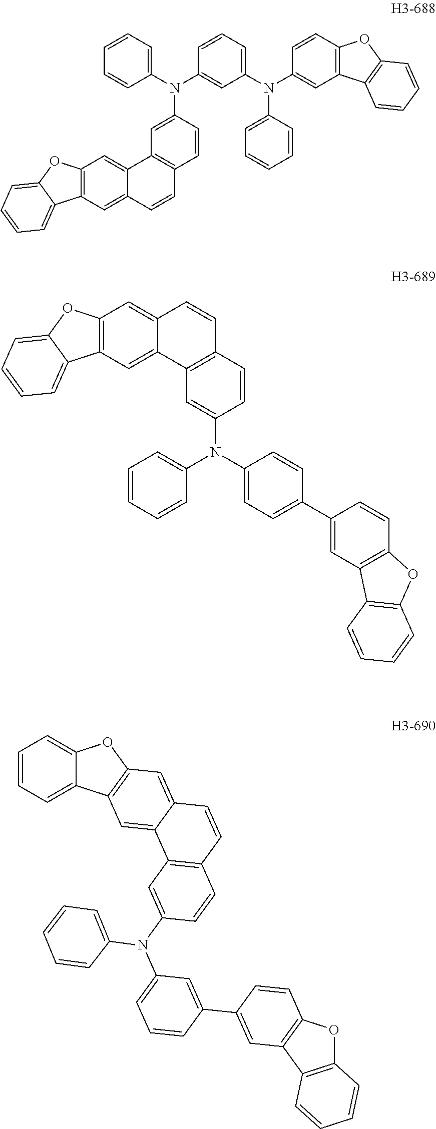 [SMILES: c1ccc(N(c2cccc(N(c3ccccc3)c3ccc4oc5ccccc5c4c3)c2)c2ccc3ccc4cc5c(cc4c3c2)oc2ccccc25)cc1 ; c1ccc(N(c2ccc(-c3ccc4oc5ccccc5c4c3)cc2)c2ccc3ccc4cc5oc6ccccc6c5cc4c3c2)cc1 ; c1ccc(N(c2cccc(-c3ccc4oc5ccccc5c4c3)c2)c2ccc3ccc4cc5oc6ccccc6c5cc4c3c2)cc1]

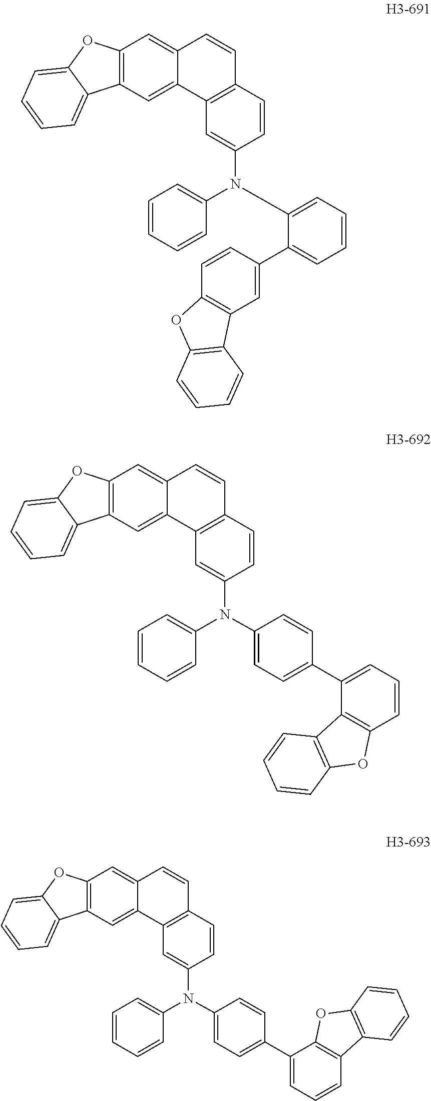 [SMILES: c1ccc(N(c2ccc3ccc4cc5oc6ccccc6c5cc4c3c2)c2ccccc2-c2ccc3oc4ccccc4c3c2)cc1 ; c1ccc(N(c2ccc(-c3cccc4oc5ccccc5c34)cc2)c2ccc3ccc4cc5oc6ccccc6c5cc4c3c2)cc1 ; c1ccc(N(c2ccc(-c3cccc4c3oc3ccccc34)cc2)c2ccc3ccc4cc5oc6ccccc6c5cc4c3c2)cc1]

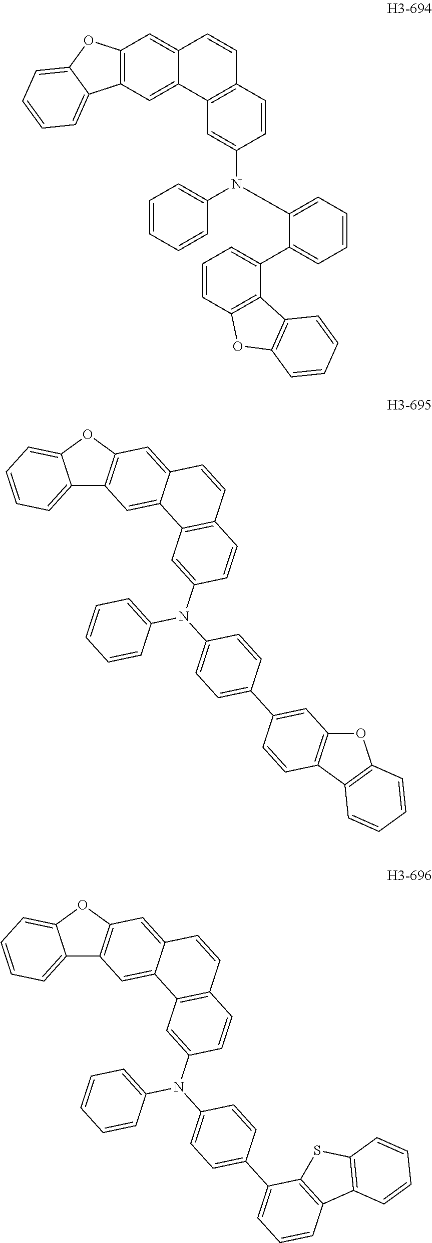 [SMILES: c1ccc(N(c2ccc3ccc4cc5oc6ccccc6c5cc4c3c2)c2ccccc2-c2cccc3oc4ccccc4c23)cc1 ; c1ccc(N(c2ccc(-c3ccc4c(c3)oc3ccccc34)cc2)c2ccc3ccc4cc5oc6ccccc6c5cc4c3c2)cc1 ; c1ccc(N(c2ccc(-c3cccc4c3sc3ccccc34)cc2)c2ccc3ccc4cc5oc6ccccc6c5cc4c3c2)cc1]

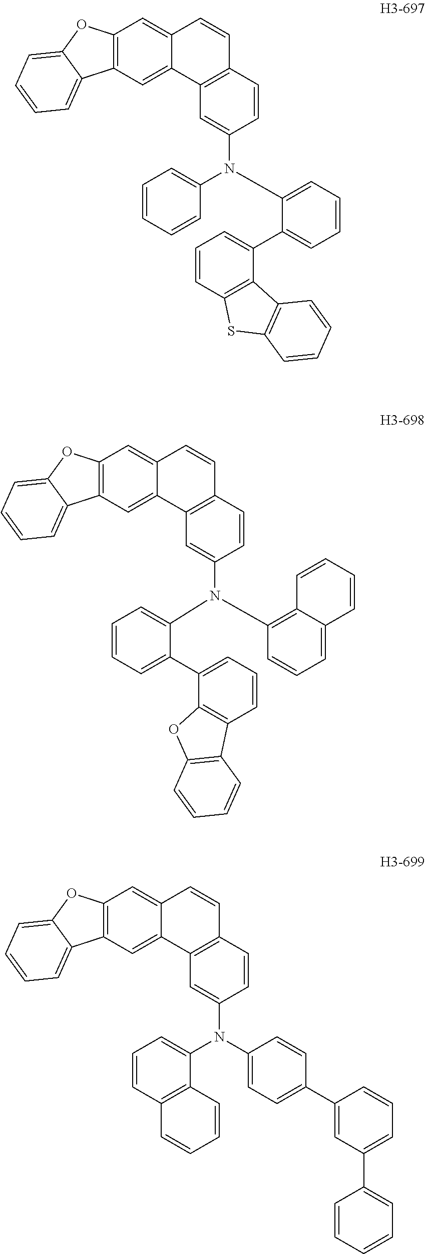 [SMILES: c1ccc(N(c2ccc3ccc4cc5oc6ccccc6c5cc4c3c2)c2ccccc2-c2cccc3sc4ccccc4c23)cc1 ; c1ccc(N(c2ccc3ccc4cc5oc6ccccc6c5cc4c3c2)c2cccc3ccccc23)c(-c2cccc3c2oc2ccccc23)c1 ; c1ccc(-c2cccc(-c3ccc(N(c4ccc5ccc6cc7oc8ccccc8c7cc6c5c4)c4cccc5ccccc45)cc3)c2)cc1]

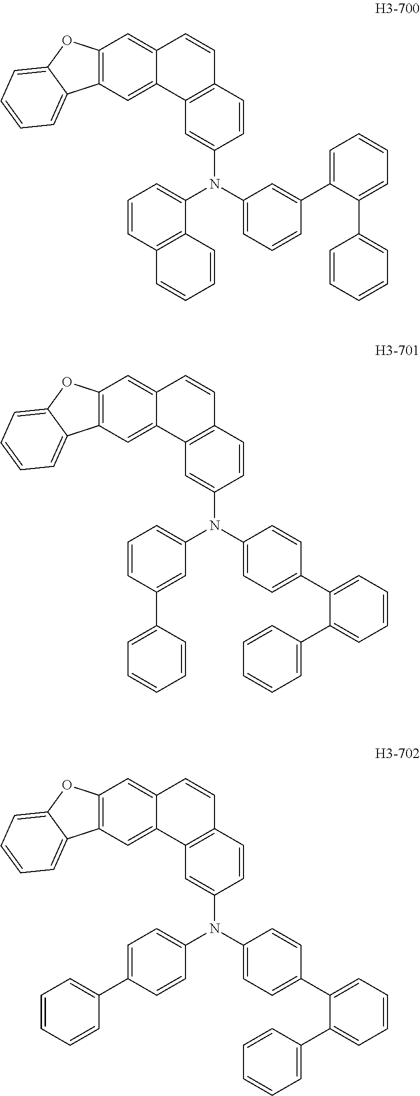 [SMILES: c1ccc(-c2ccccc2-c2cccc(N(c3ccc4ccc5cc6oc7ccccc7c6cc5c4c3)c3cccc4ccccc34)c2)cc1 ; c1ccc(-c2cccc(N(c3ccc(-c4ccccc4-c4ccccc4)cc3)c3ccc4ccc5cc6oc7ccccc7c6cc5c4c3)c2)cc1 ; c1ccc(-c2ccc(N(c3ccc(-c4ccccc4-c4ccccc4)cc3)c3ccc4ccc5cc6oc7ccccc7c6cc5c4c3)cc2)cc1]

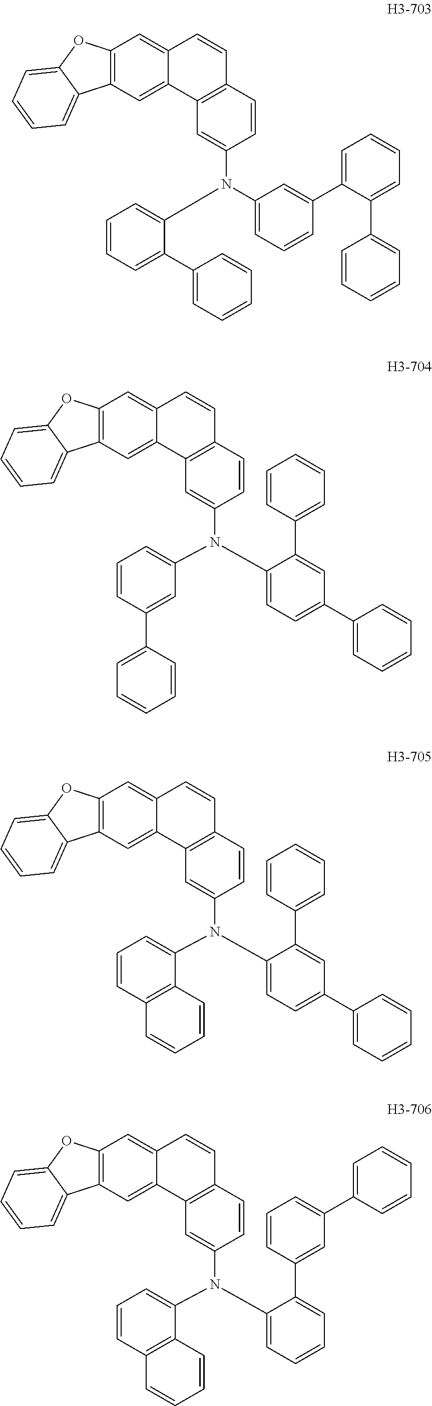 [SMILES: c1ccc(-c2ccccc2-c2cccc(N(c3ccc4ccc5cc6oc7ccccc7c6cc5c4c3)c3ccccc3-c3ccccc3)c2)cc1 ; c1ccc(-c2cccc(N(c3ccc4ccc5cc6oc7ccccc7c6cc5c4c3)c3ccc(-c4ccccc4)cc3-c3ccccc3)c2)cc1 ; c1ccc(-c2ccc(N(c3ccc4ccc5cc6oc7ccccc7c6cc5c4c3)c3cccc4ccccc34)c(-c3ccccc3)c2)cc1 ; c1ccc(-c2cccc(-c3ccccc3N(c3ccc4ccc5cc6oc7ccccc7c6cc5c4c3)c3cccc4ccccc34)c2)cc1]

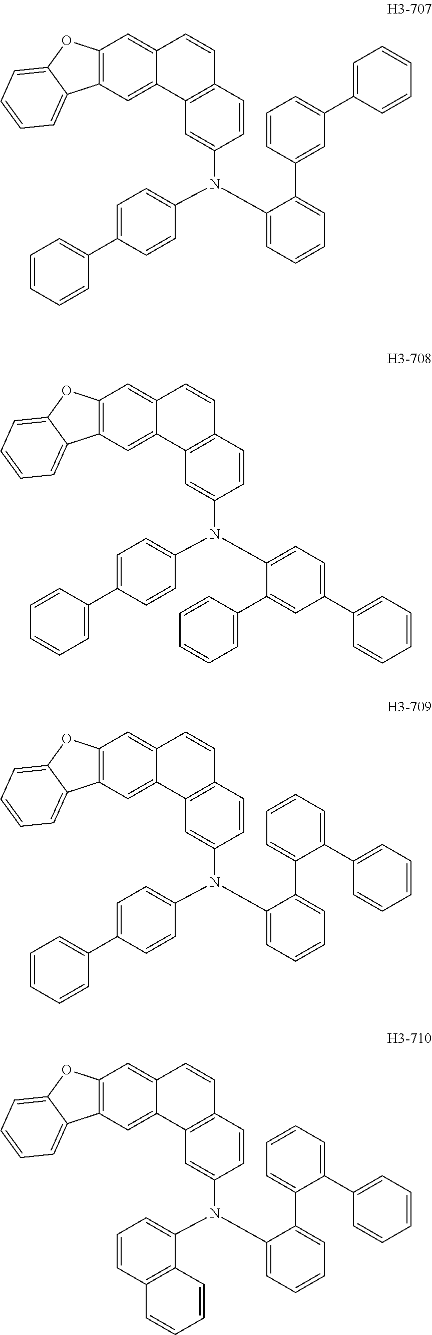 [SMILES: c1ccc(-c2ccc(N(c3ccc4ccc5cc6oc7ccccc7c6cc5c4c3)c3ccccc3-c3cccc(-c4ccccc4)c3)cc2)cc1 ; c1ccc(-c2ccc(N(c3ccc4ccc5cc6oc7ccccc7c6cc5c4c3)c3ccc(-c4ccccc4)cc3-c3ccccc3)cc2)cc1 ; c1ccc(-c2ccc(N(c3ccc4ccc5cc6oc7ccccc7c6cc5c4c3)c3ccccc3-c3ccccc3-c3ccccc3)cc2)cc1 ; c1ccc(-c2ccccc2-c2ccccc2N(c2ccc3ccc4cc5oc6ccccc6c5cc4c3c2)c2cccc3ccccc23)cc1]

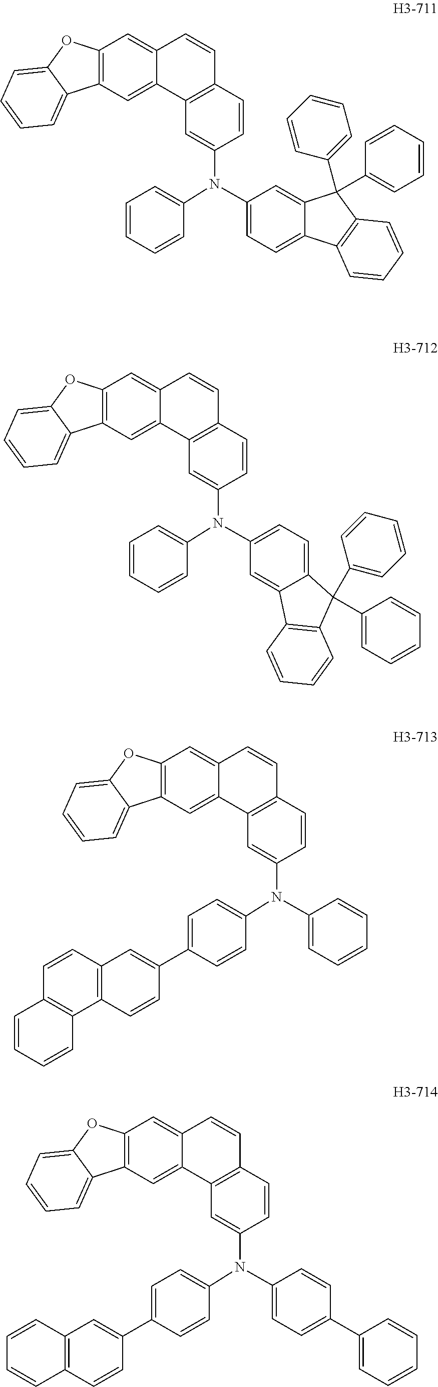 [SMILES: c1ccc(N(c2ccc3c(c2)C(c2ccccc2)(c2ccccc2)c2ccccc2-3)c2ccc3ccc4cc5oc6ccccc6c5cc4c3c2)cc1 ; c1ccc(N(c2ccc3c(c2)-c2ccccc2C3(c2ccccc2)c2ccccc2)c2ccc3ccc4cc5oc6ccccc6c5cc4c3c2)cc1 ; c1ccc(N(c2ccc(-c3ccc4c(ccc5ccccc54)c3)cc2)c2ccc3ccc4cc5oc6ccccc6c5cc4c3c2)cc1 ; c1ccc(-c2ccc(N(c3ccc(-c4ccc5ccccc5c4)cc3)c3ccc4ccc5cc6oc7ccccc7c6cc5c4c3)cc2)cc1]

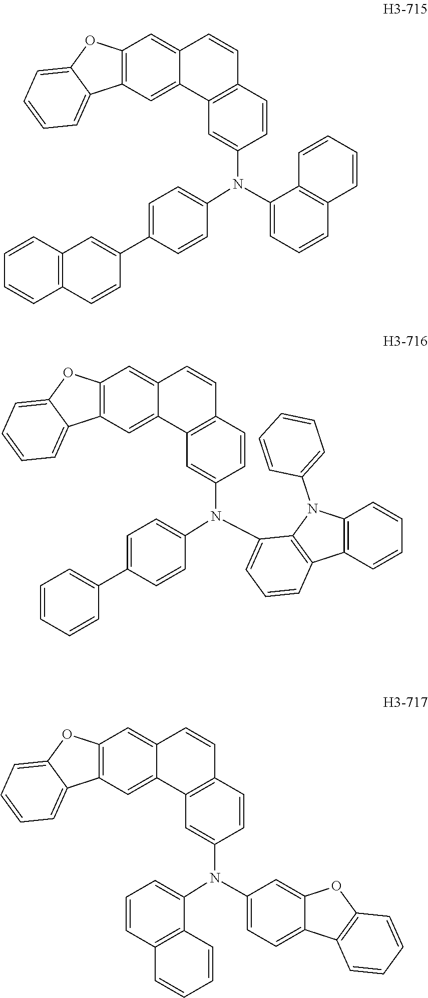 [SMILES: c1ccc2cc(-c3ccc(N(c4ccc5ccc6cc7oc8ccccc8c7cc6c5c4)c4cccc5ccccc45)cc3)ccc2c1 ; c1ccc(-c2ccc(N(c3ccc4ccc5cc6oc7ccccc7c6cc5c4c3)c3cccc4c5ccccc5n(-c5ccccc5)c34)cc2)cc1 ; c1ccc2c(N(c3ccc4c(c3)oc3ccccc34)c3ccc4ccc5cc6oc7ccccc7c6cc5c4c3)cccc2c1]

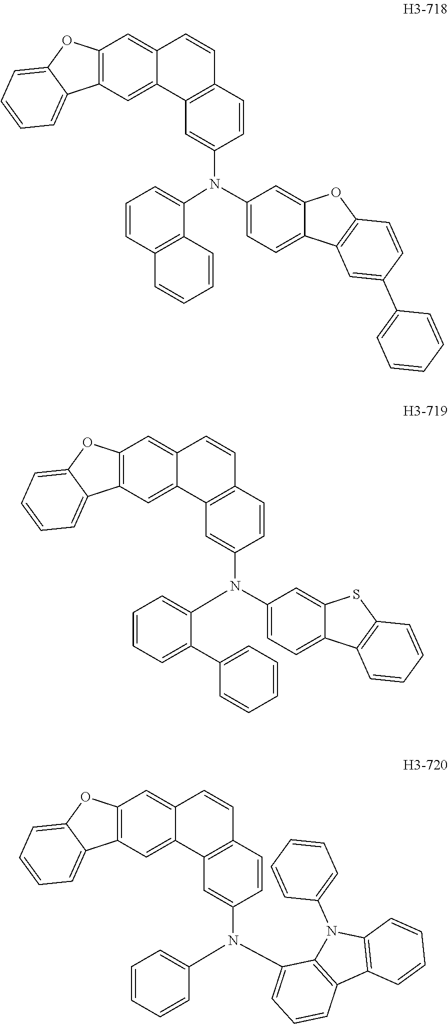 [SMILES: c1ccc(-c2ccc3oc4cc(N(c5ccc6ccc7cc8oc9ccccc9c8cc7c6c5)c5cccc6ccccc56)ccc4c3c2)cc1 ; c1ccc(-c2ccccc2N(c2ccc3c(c2)sc2ccccc23)c2ccc3ccc4cc5oc6ccccc6c5cc4c3c2)cc1 ; c1ccc(N(c2ccc3ccc4cc5oc6ccccc6c5cc4c3c2)c2cccc3c4ccccc4n(-c4ccccc4)c23)cc1]

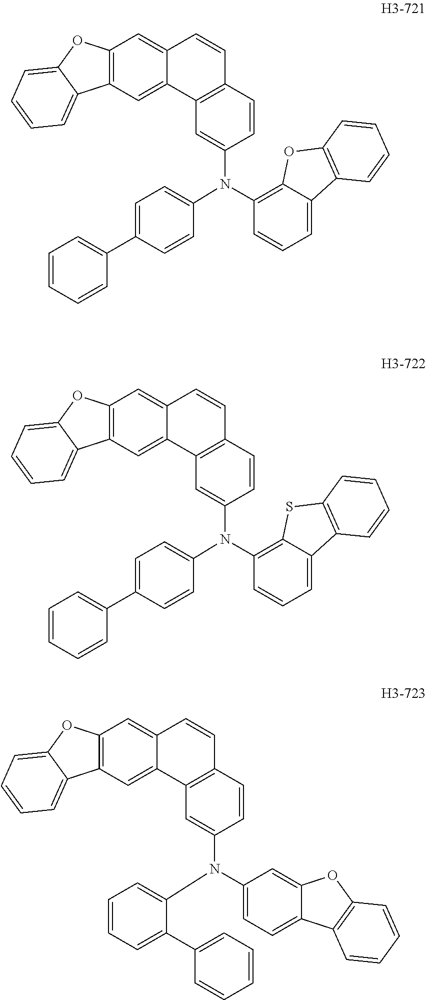 [SMILES: c1ccc(-c2ccc(N(c3ccc4ccc5cc6oc7ccccc7c6cc5c4c3)c3cccc4c3oc3ccccc34)cc2)cc1 ; c1ccc(-c2ccc(N(c3ccc4ccc5cc6oc7ccccc7c6cc5c4c3)c3cccc4c3sc3ccccc34)cc2)cc1 ; c1ccc(-c2ccccc2N(c2ccc3c(c2)oc2ccccc23)c2ccc3ccc4cc5oc6ccccc6c5cc4c3c2)cc1]

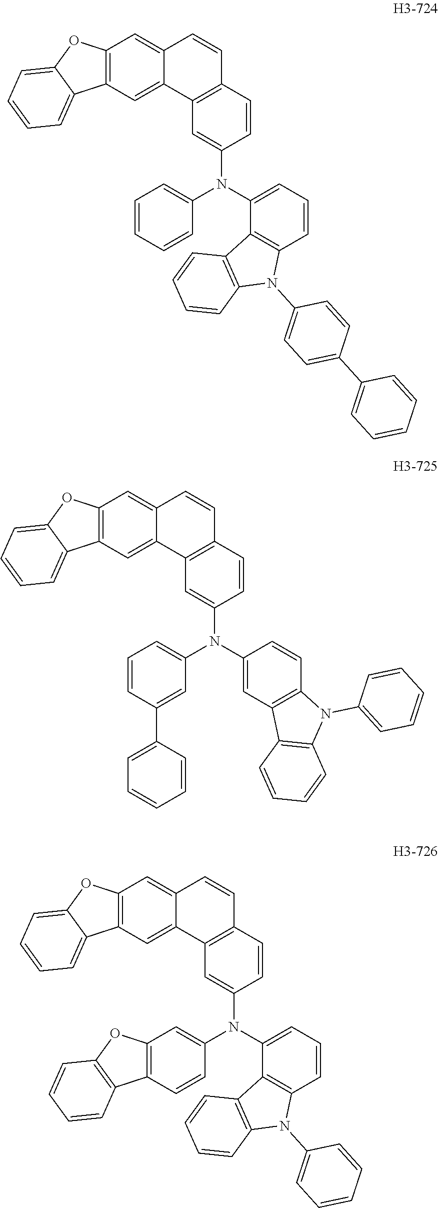 [SMILES: c1ccc(-c2ccc(-n3c4ccccc4c4c(N(c5ccccc5)c5ccc6ccc7cc8oc9ccccc9c8cc7c6c5)cccc43)cc2)cc1 ; c1ccc(-c2cccc(N(c3ccc4ccc5cc6oc7ccccc7c6cc5c4c3)c3ccc4c(c3)c3ccccc3n4-c3ccccc3)c2)cc1 ; c1ccc(-n2c3ccccc3c3c(N(c4ccc5c(c4)oc4ccccc45)c4ccc5ccc6cc7oc8ccccc8c7cc6c5c4)cccc32)cc1]

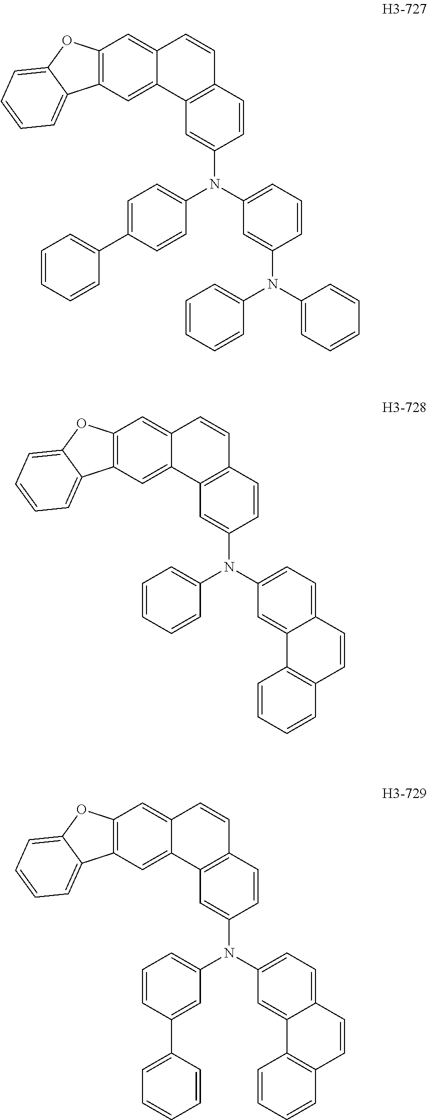 [SMILES: c1ccc(-c2ccc(N(c3cccc(N(c4ccccc4)c4ccccc4)c3)c3ccc4ccc5cc6oc7ccccc7c6cc5c4c3)cc2)cc1 ; c1ccc(N(c2ccc3ccc4ccccc4c3c2)c2ccc3ccc4cc5oc6ccccc6c5cc4c3c2)cc1 ; c1ccc(-c2cccc(N(c3ccc4ccc5ccccc5c4c3)c3ccc4ccc5cc6oc7ccccc7c6cc5c4c3)c2)cc1]

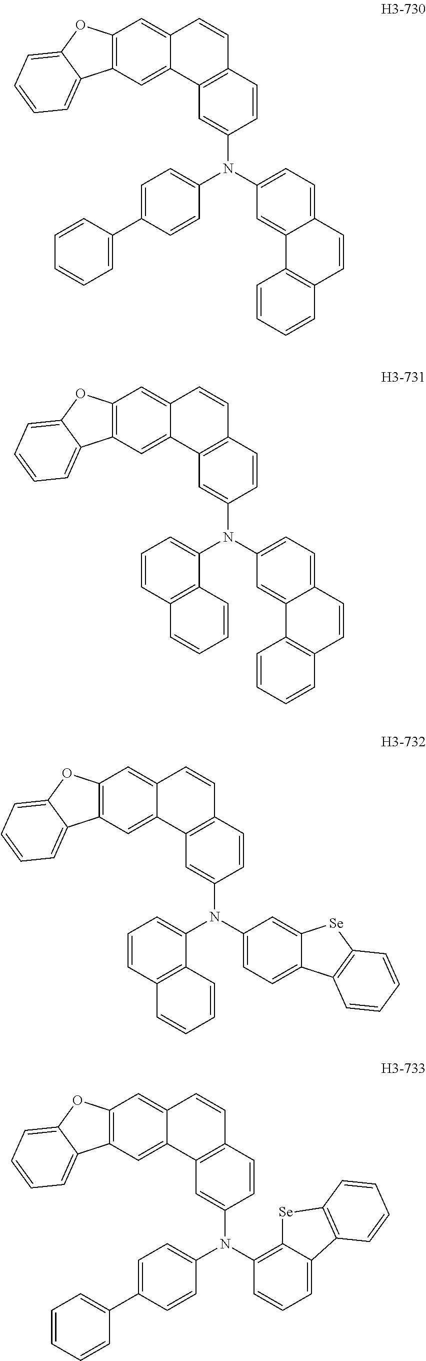 [SMILES: c1ccc(-c2ccc(N(c3ccc4ccc5ccccc5c4c3)c3ccc4ccc5cc6oc7ccccc7c6cc5c4c3)cc2)cc1 ; c1ccc2c(N(c3ccc4ccc5ccccc5c4c3)c3ccc4ccc5cc6oc7ccccc7c6cc5c4c3)cccc2c1 ; c1ccc2c(N(c3ccc4c(c3)[se]c3ccccc34)c3ccc4ccc5cc6oc7ccccc7c6cc5c4c3)cccc2c1 ; c1ccc(-c2ccc(N(c3ccc4ccc5cc6oc7ccccc7c6cc5c4c3)c3cccc4c3[se]c3ccccc34)cc2)cc1]

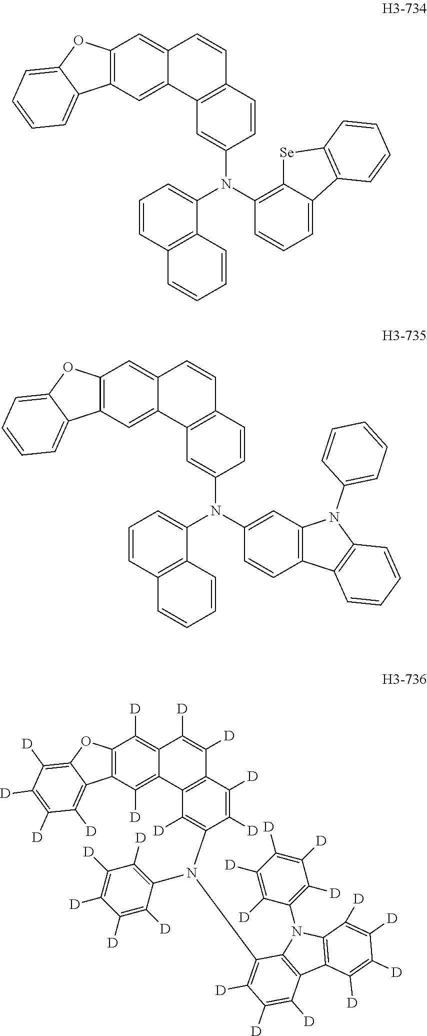 [SMILES: c1ccc2c(N(c3ccc4ccc5cc6oc7ccccc7c6cc5c4c3)c3cccc4c3[se]c3ccccc34)cccc2c1 ; c1ccc(-n2c3ccccc3c3ccc(N(c4ccc5ccc6cc7oc8ccccc8c7cc6c5c4)c4cccc5ccccc45)cc32)cc1 ; [2H]c1c([2H])c([2H])c(N(c2c([2H])c([2H])c3c([2H])c([2H])c4c([2H])c5oc6c([2H])c([2H])c([2H])c([2H])c6c5c([2H])c4c3c2[2H])c2c([2H])c([2H])c([2H])c3c4c([2H])c([2H])c([2H])c([2H])c4n(-c4c([2H])c([2H])c([2H])c([2H])c4[2H])c23)c([2H])c1[2H]]

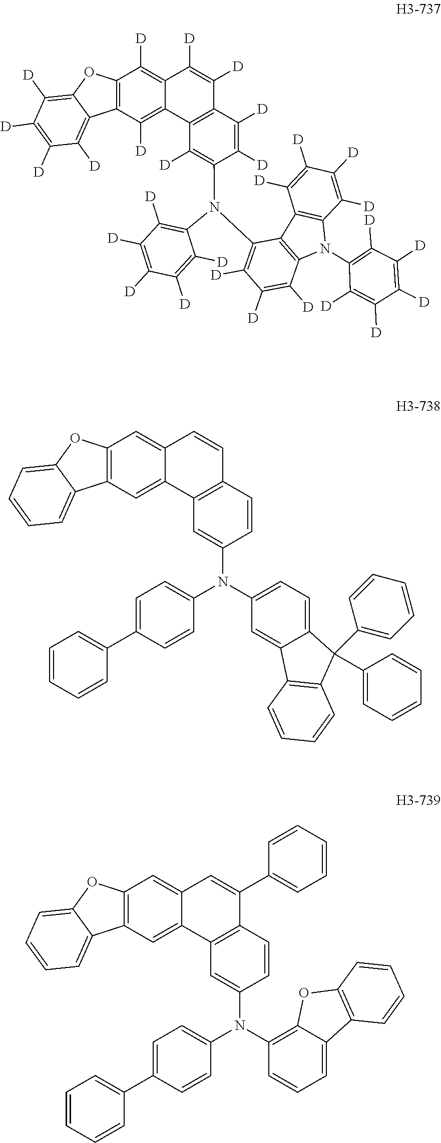 [SMILES: [2H]c1c([2H])c([2H])c(N(c2c([2H])c([2H])c3c([2H])c([2H])c4c([2H])c5oc6c([2H])c([2H])c([2H])c([2H])c6c5c([2H])c4c3c2[2H])c2c([2H])c([2H])c([2H])c3c2c2c([2H])c([2H])c([2H])c([2H])c2n3-c2c([2H])c([2H])c([2H])c([2H])c2[2H])c([2H])c1[2H] ; c1ccc(-c2ccc(N(c3ccc4c(c3)-c3ccccc3C4(c3ccccc3)c3ccccc3)c3ccc4ccc5cc6oc7ccccc7c6cc5c4c3)cc2)cc1 ; c1ccc(-c2ccc(N(c3ccc4c(-c5ccccc5)cc5cc6oc7ccccc7c6cc5c4c3)c3cccc4c3oc3ccccc34)cc2)cc1]

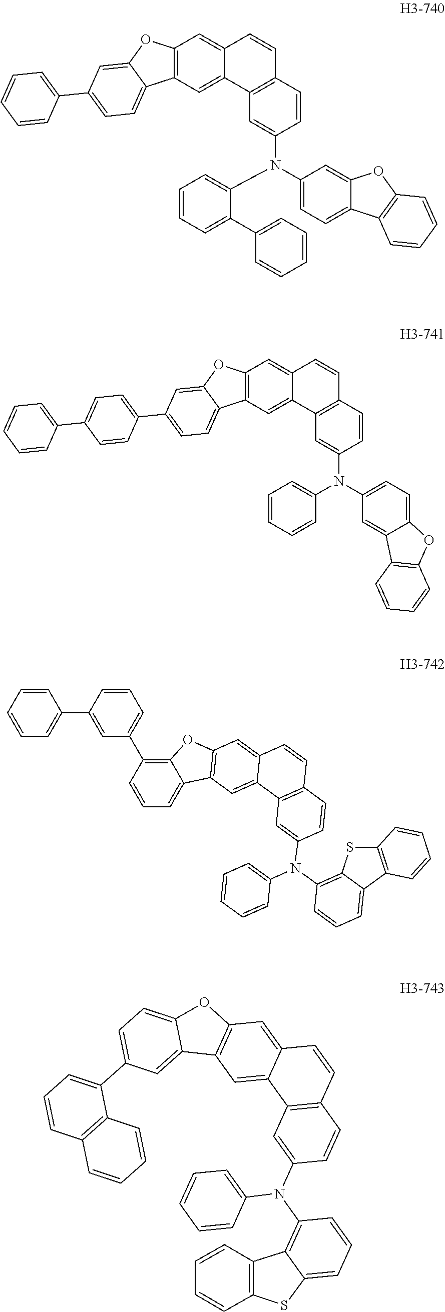 [SMILES: c1ccc(-c2ccc3c(c2)oc2cc4ccc5ccc(N(c6ccc7c(c6)oc6ccccc67)c6ccccc6-c6ccccc6)cc5c4cc23)cc1 ; c1ccc(-c2ccc(-c3ccc4c(c3)oc3cc5ccc6ccc(N(c7ccccc7)c7ccc8oc9ccccc9c8c7)cc6c5cc34)cc2)cc1 ; c1ccc(-c2cccc(-c3cccc4c3oc3cc5ccc6ccc(N(c7ccccc7)c7cccc8c7sc7ccccc78)cc6c5cc34)c2)cc1 ; c1ccc(N(c2ccc3ccc4cc5oc6ccc(-c7cccc8ccccc78)cc6c5cc4c3c2)c2cccc3sc4ccccc4c23)cc1]

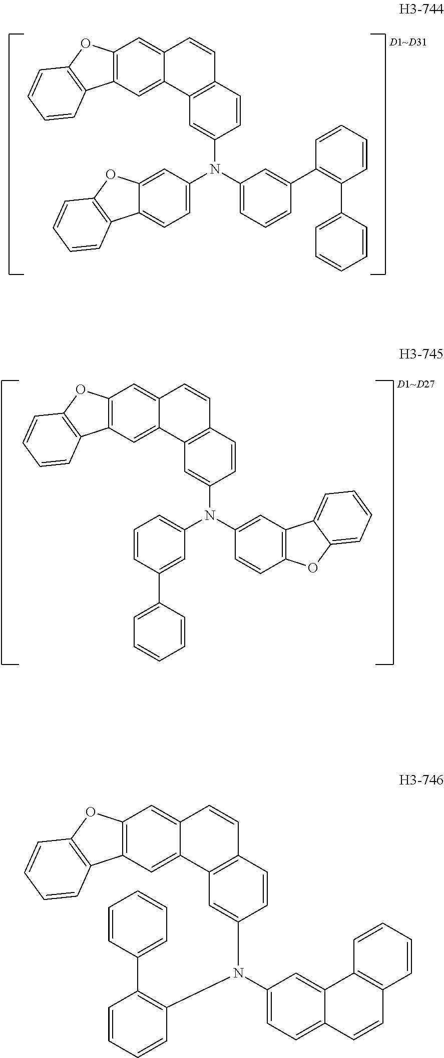

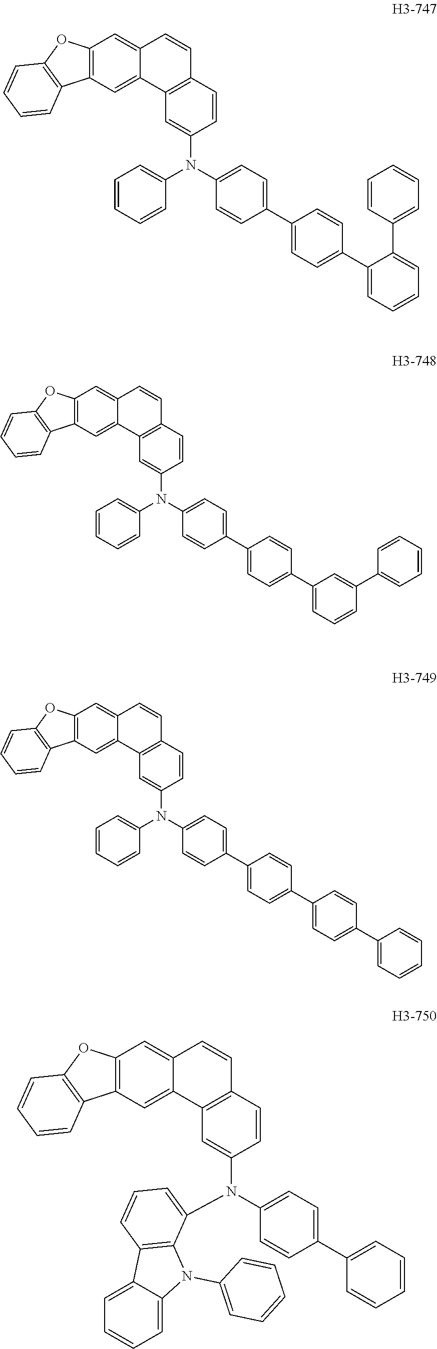 [SMILES: c1ccc(-c2ccccc2-c2ccc(-c3ccc(N(c4ccccc4)c4ccc5ccc6cc7oc8ccccc8c7cc6c5c4)cc3)cc2)cc1 ; c1ccc(-c2cccc(-c3ccc(-c4ccc(N(c5ccccc5)c5ccc6ccc7cc8oc9ccccc9c8cc7c6c5)cc4)cc3)c2)cc1 ; c1ccc(-c2ccc(-c3ccc(-c4ccc(N(c5ccccc5)c5ccc6ccc7cc8oc9ccccc9c8cc7c6c5)cc4)cc3)cc2)cc1 ; c1ccc(-c2ccc(N(c3ccc4ccc5cc6oc7ccccc7c6cc5c4c3)c3cccc4c5ccccc5n(-c5ccccc5)c34)cc2)cc1]

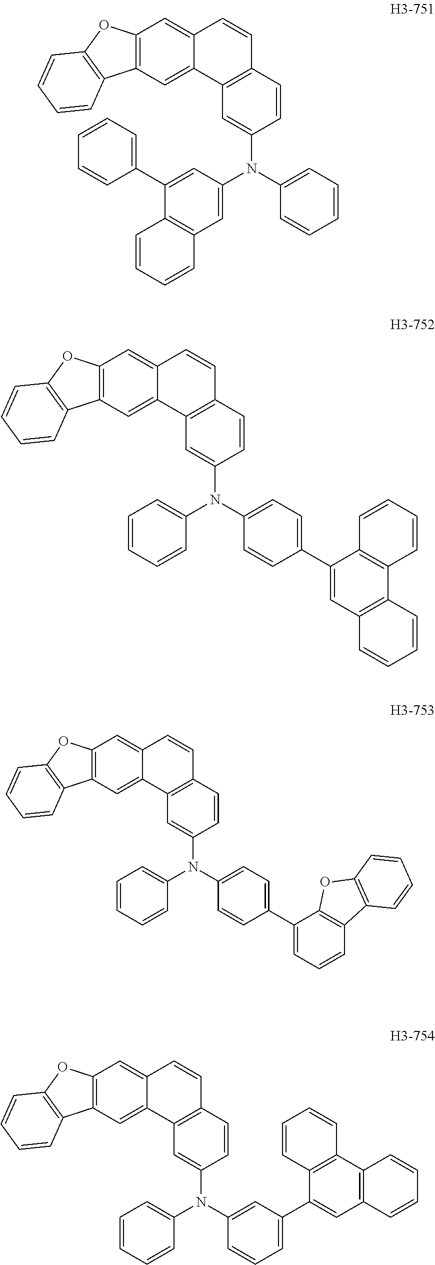 [SMILES: c1ccc(-c2cc(N(c3ccccc3)c3ccc4ccc5cc6oc7ccccc7c6cc5c4c3)cc3ccccc23)cc1 ; c1ccc(N(c2ccc(-c3cc4ccccc4c4ccccc34)cc2)c2ccc3ccc4cc5oc6ccccc6c5cc4c3c2)cc1 ; c1ccc(N(c2ccc(-c3cccc4c3oc3ccccc34)cc2)c2ccc3ccc4cc5oc6ccccc6c5cc4c3c2)cc1 ; c1ccc(N(c2cccc(-c3cc4ccccc4c4ccccc34)c2)c2ccc3ccc4cc5oc6ccccc6c5cc4c3c2)cc1]

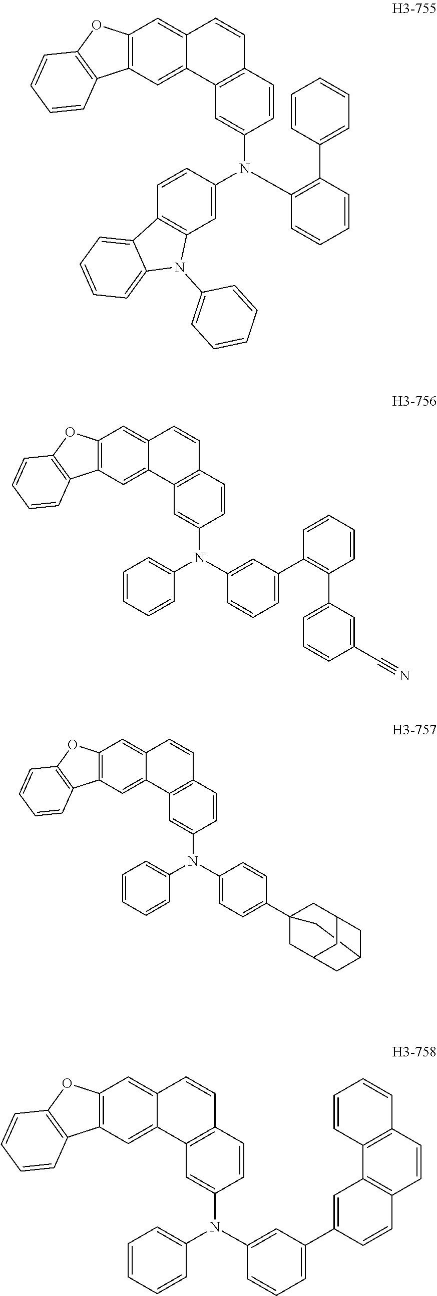 [SMILES: c1ccc(-c2ccccc2N(c2ccc3ccc4cc5oc6ccccc6c5cc4c3c2)c2ccc3c4ccccc4n(-c4ccccc4)c3c2)cc1 ; N#Cc1cccc(-c2ccccc2-c2cccc(N(c3ccccc3)c3ccc4ccc5cc6oc7ccccc7c6cc5c4c3)c2)c1 ; c1ccc(N(c2ccc(C34CC5CC(CC(C5)C3)C4)cc2)c2ccc3ccc4cc5oc6ccccc6c5cc4c3c2)cc1 ; c1ccc(N(c2cccc(-c3ccc4ccc5ccccc5c4c3)c2)c2ccc3ccc4cc5oc6ccccc6c5cc4c3c2)cc1]

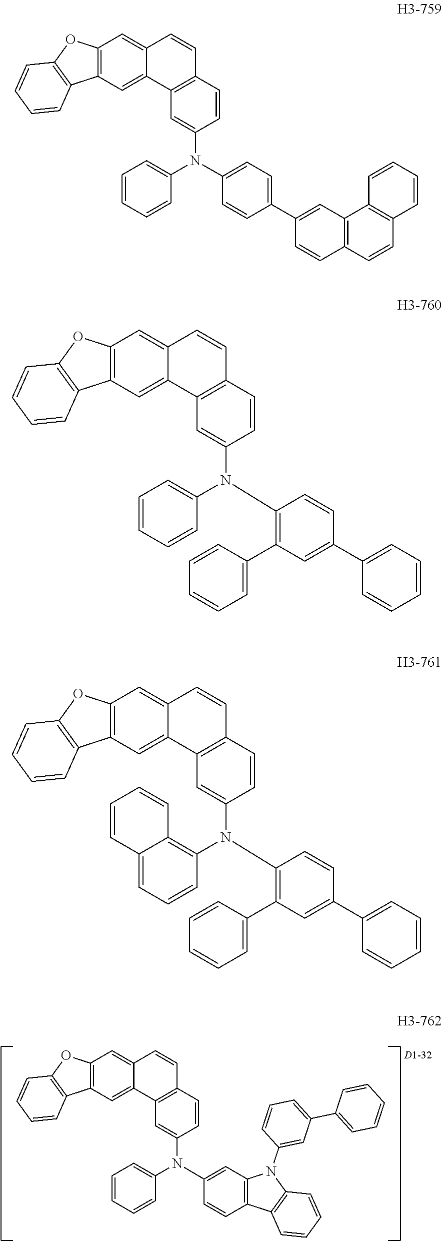

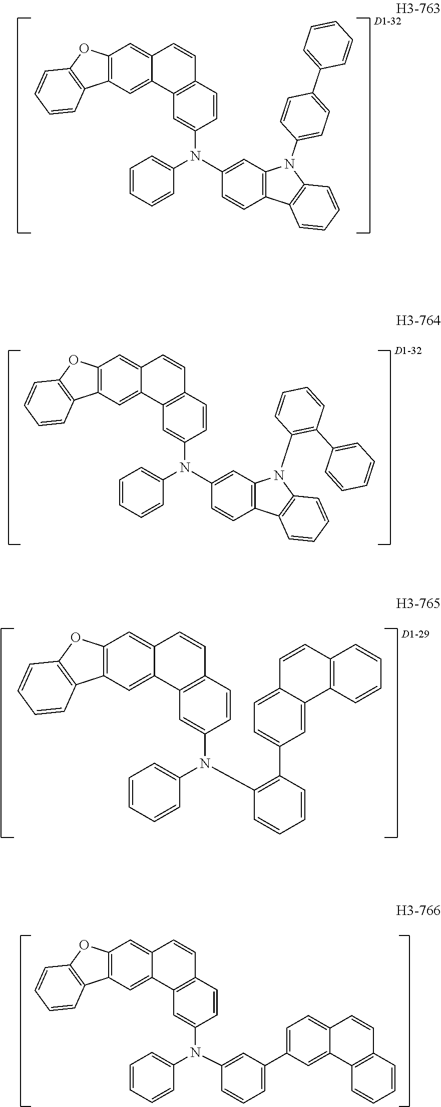

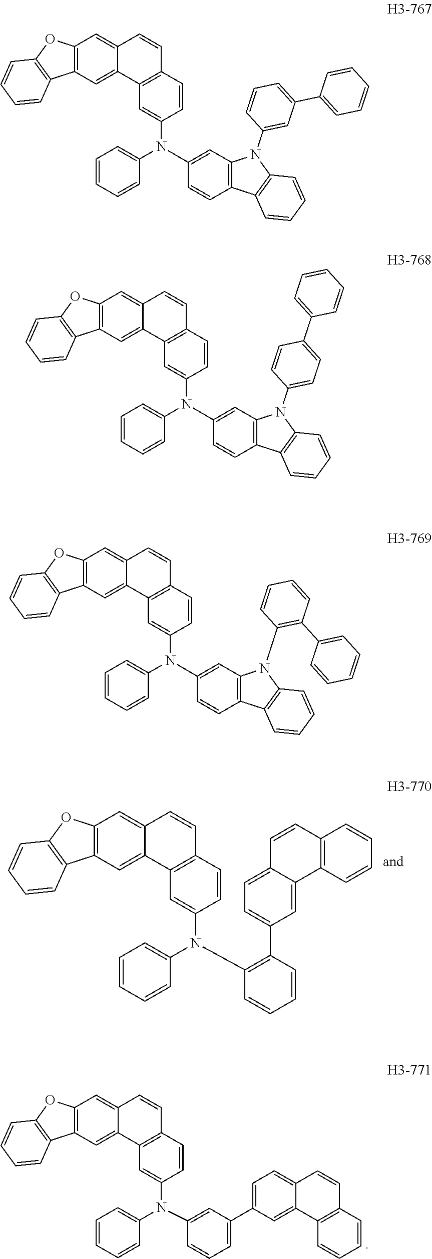 [SMILES: c1ccc(-c2cccc(-n3c4ccccc4c4ccc(N(c5ccccc5)c5ccc6ccc7cc8oc9ccccc9c8cc7c6c5)cc43)c2)cc1 ; c1ccc(-c2ccc(-n3c4ccccc4c4ccc(N(c5ccccc5)c5ccc6ccc7cc8oc9ccccc9c8cc7c6c5)cc43)cc2)cc1 ; c1ccc(-c2ccccc2-n2c3ccccc3c3ccc(N(c4ccccc4)c4ccc5ccc6cc7oc8ccccc8c7cc6c5c4)cc32)cc1 ; c1ccc(N(c2ccc3ccc4cc5oc6ccccc6c5cc4c3c2)c2ccccc2-c2ccc3ccc4ccccc4c3c2)cc1 ; c1ccc(N(c2cccc(-c3ccc4ccc5ccccc5c4c3)c2)c2ccc3ccc4cc5oc6ccccc6c5cc4c3c2)cc1]

14. An organic electroluminescent device comprising an anode; a cathode; and at least one light-emitting layer between the anode and the cathode, wherein at least one layer of the light-emitting layers comprises the plurality of host materials according to claim 10.

15. An organic electroluminescent compound represented by the following formula 1′: 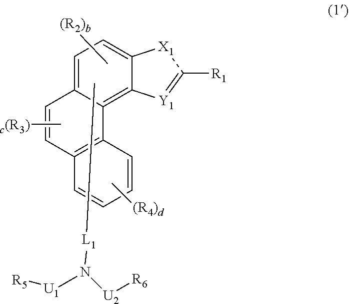
in formula 1,
X₁ and Y₁ each independently represent —N═, —NR₇—, —O— or —S—, with a proviso that one of X₁ and Y₁ represents —N═, the other of X₁ and Y₁ represents —NR₇—, —O— or —S—;
R₁ represents a substituted or unsubstituted (C6-C30)aryl, or a substituted or unsubstituted (3- to 30-membered)heteroaryl;
R₂ to R₇ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino, or may be linked to an adjacent substituent(s) to form a ring(s);
L₁, U₁, and U₂ each independently represent a single bond, a substituted or unsubstituted (C6-C30)arylene, or a substituted or unsubstituted (3- to 30-membered)heteroarylene;
b and c each independently represent an integer of 1 or 2, d represents an integer of 1 to 4, where if b to d represent an integer of 2 or more, each of R₂ to each of R₄ may be the same as or different from each other; and
in formula 1′, at least one deuterium is included.

16. The organic electroluminescent compound according to claim 15, wherein the organic electroluminescent compound represented by formula 1′ is selected from the following compounds: 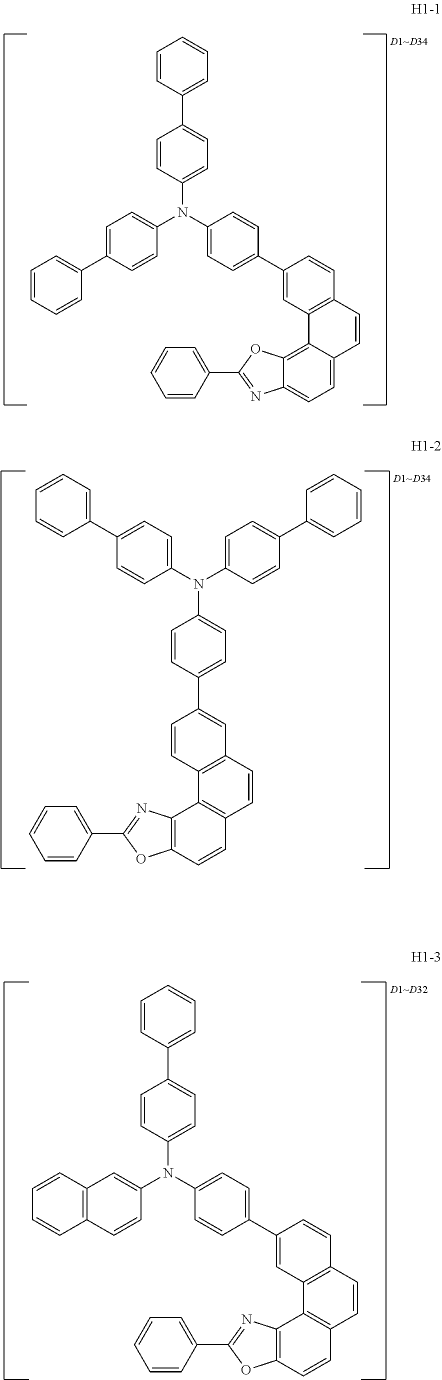 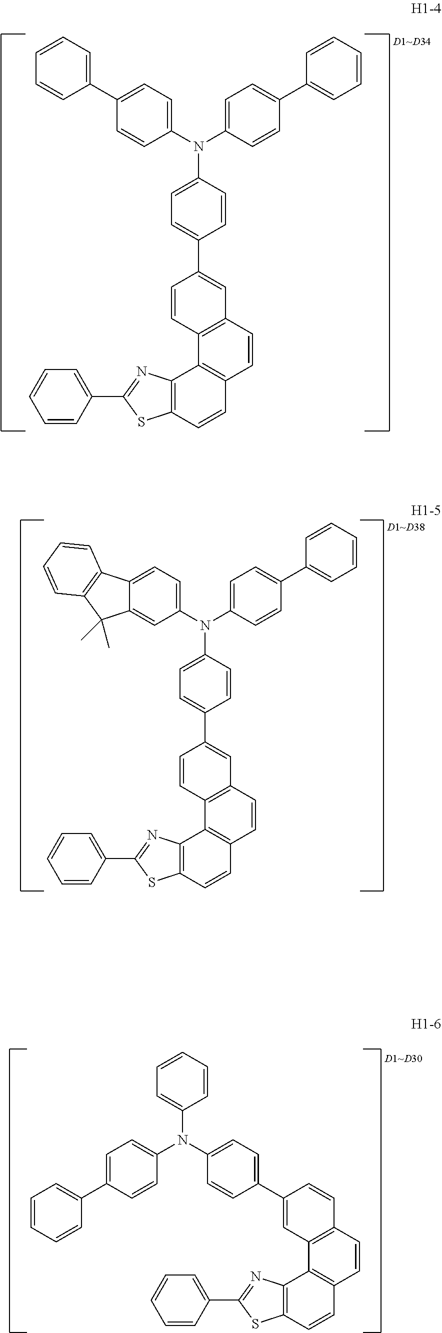 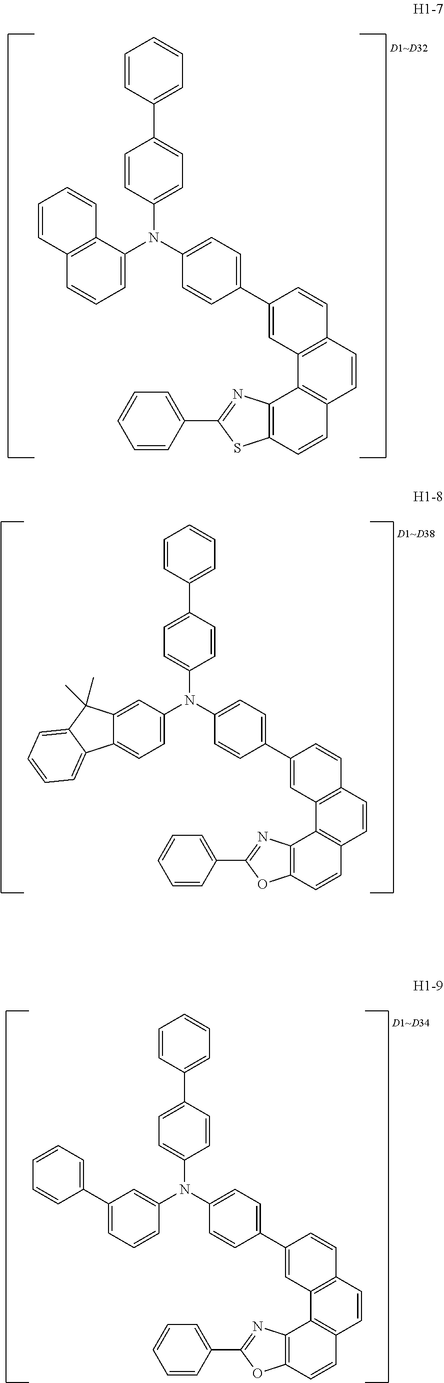 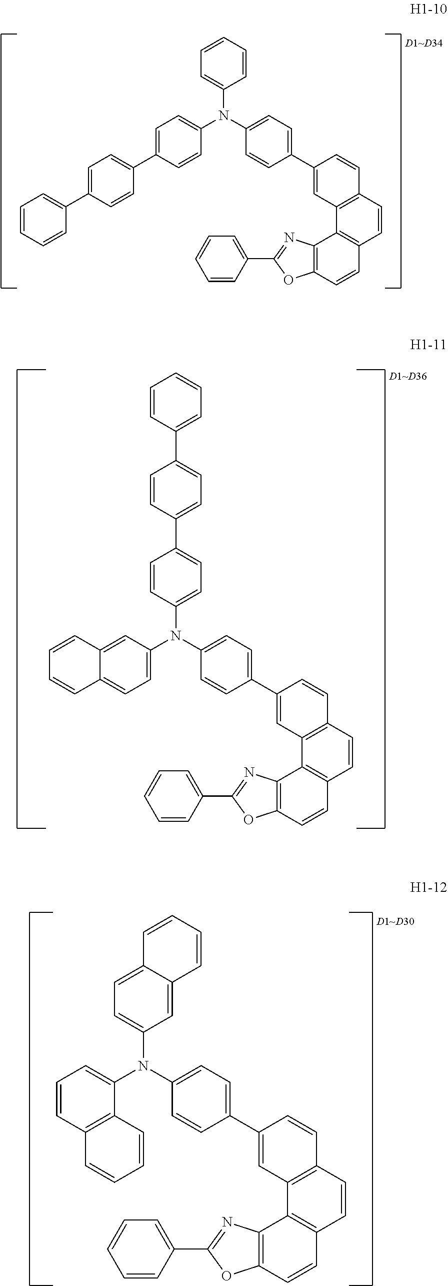 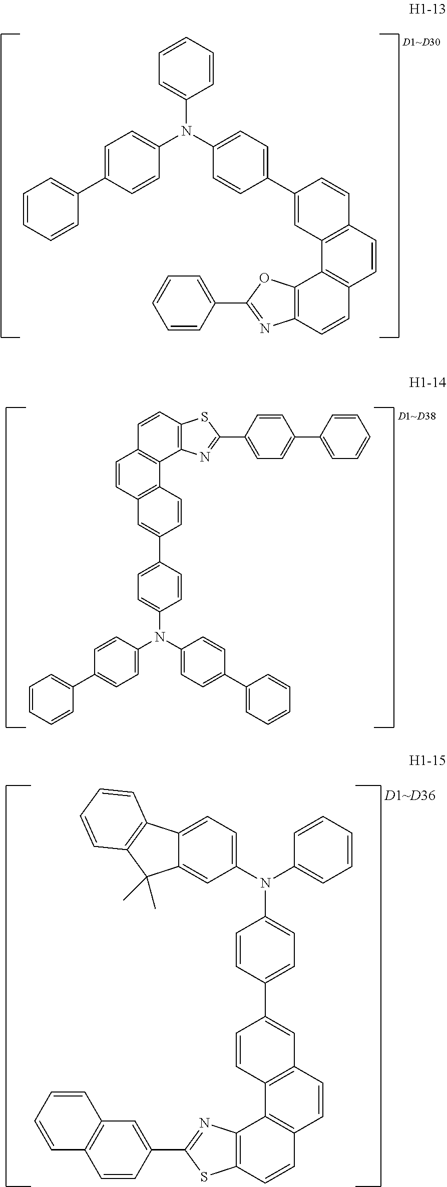 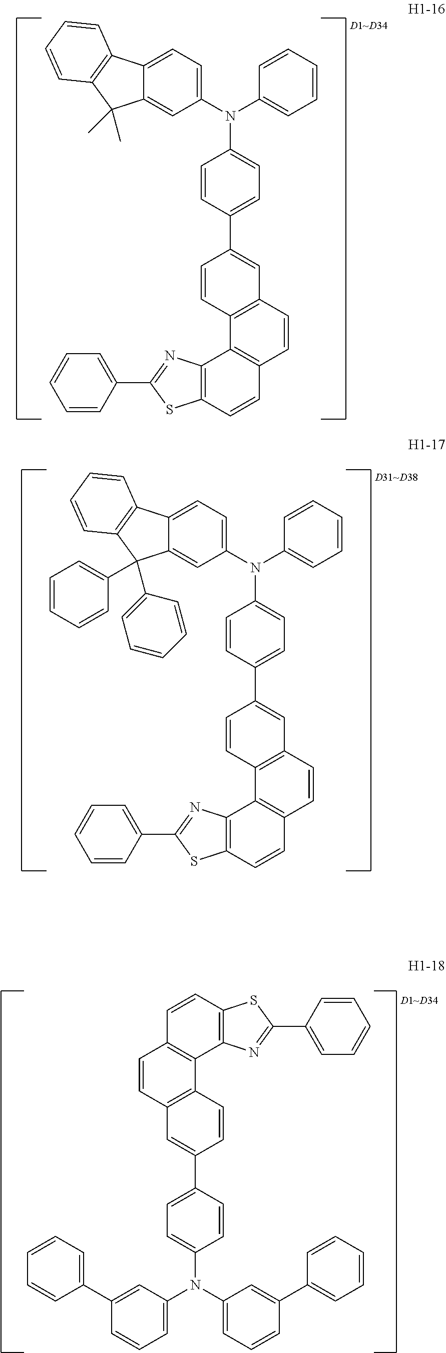 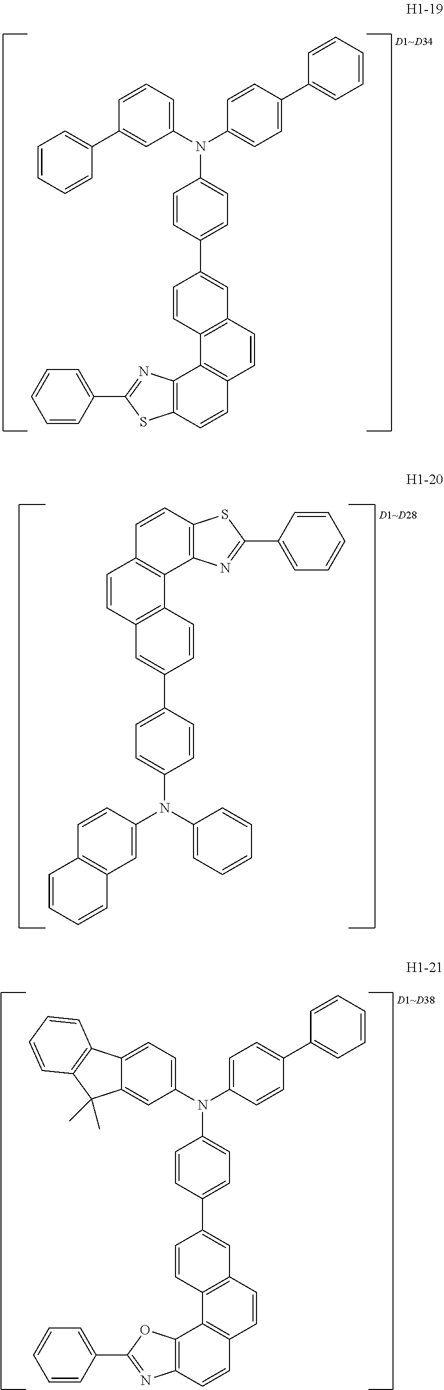 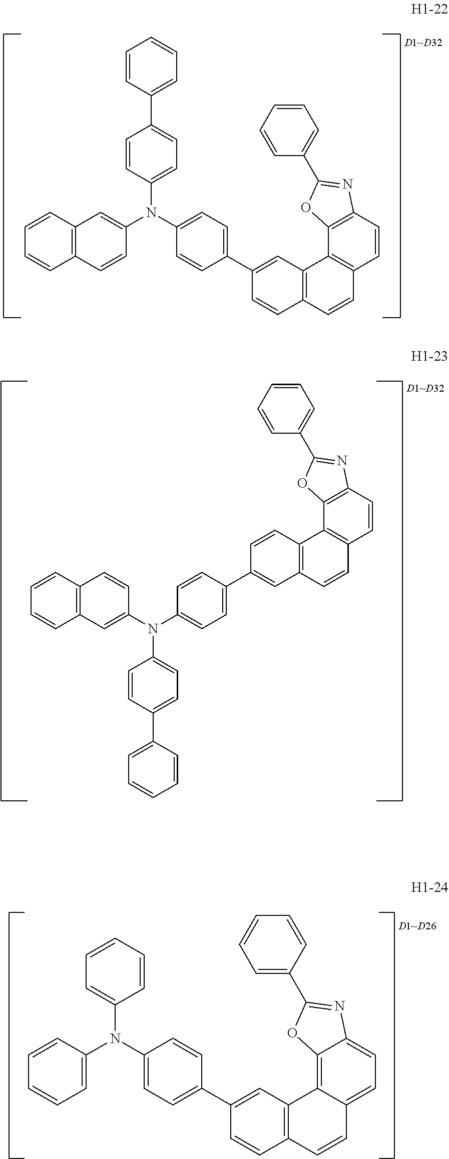 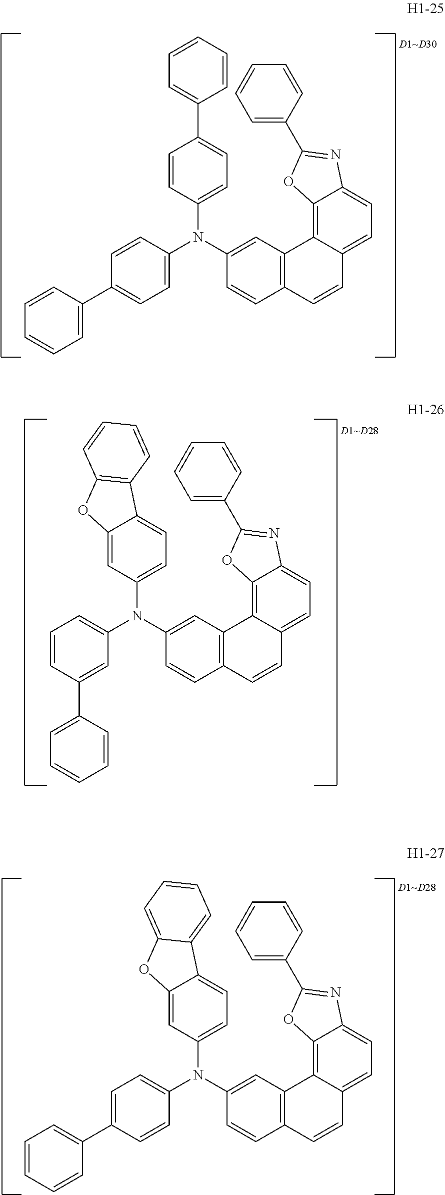 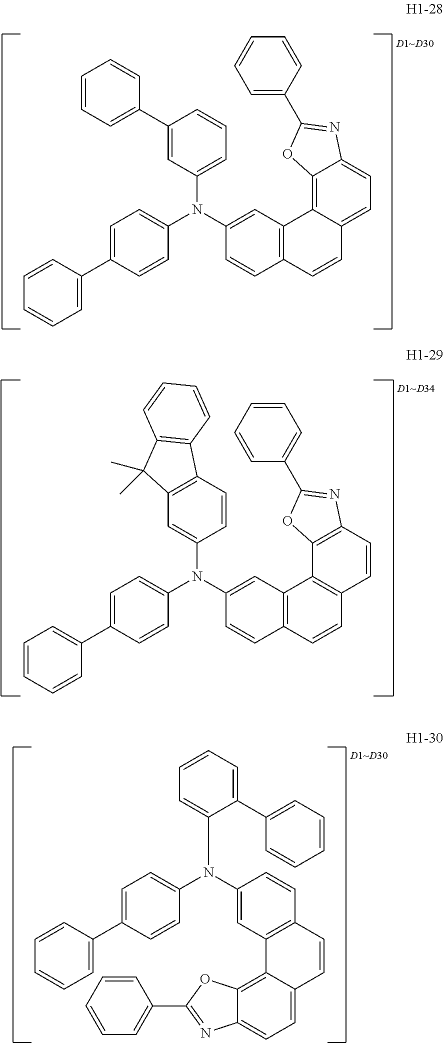 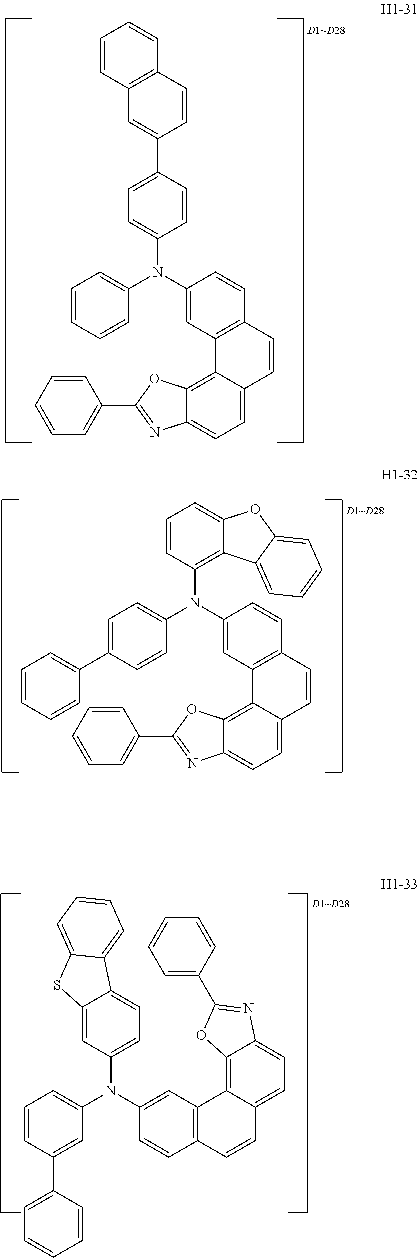 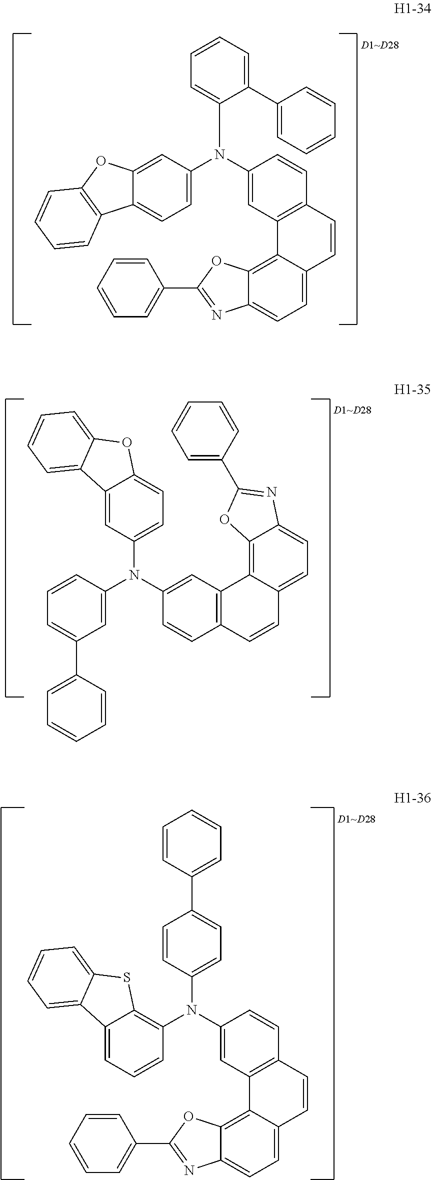 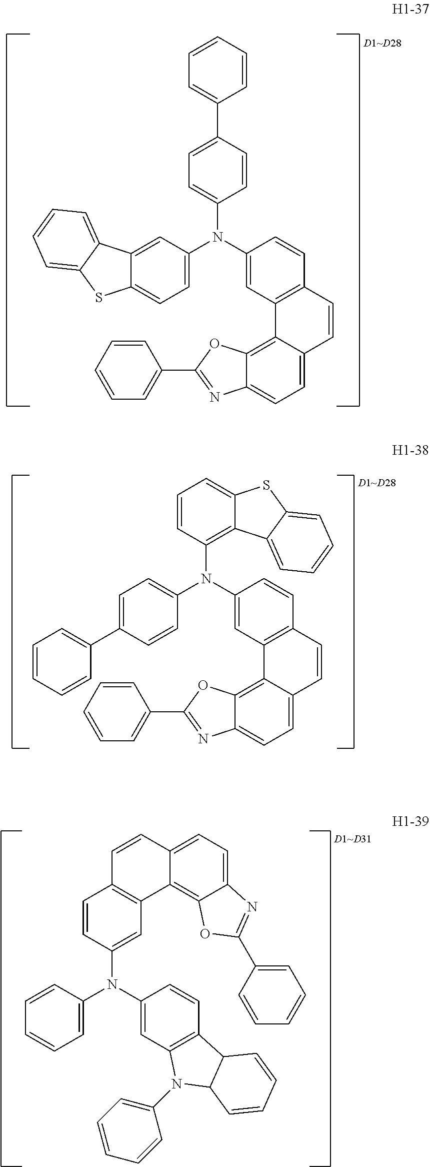 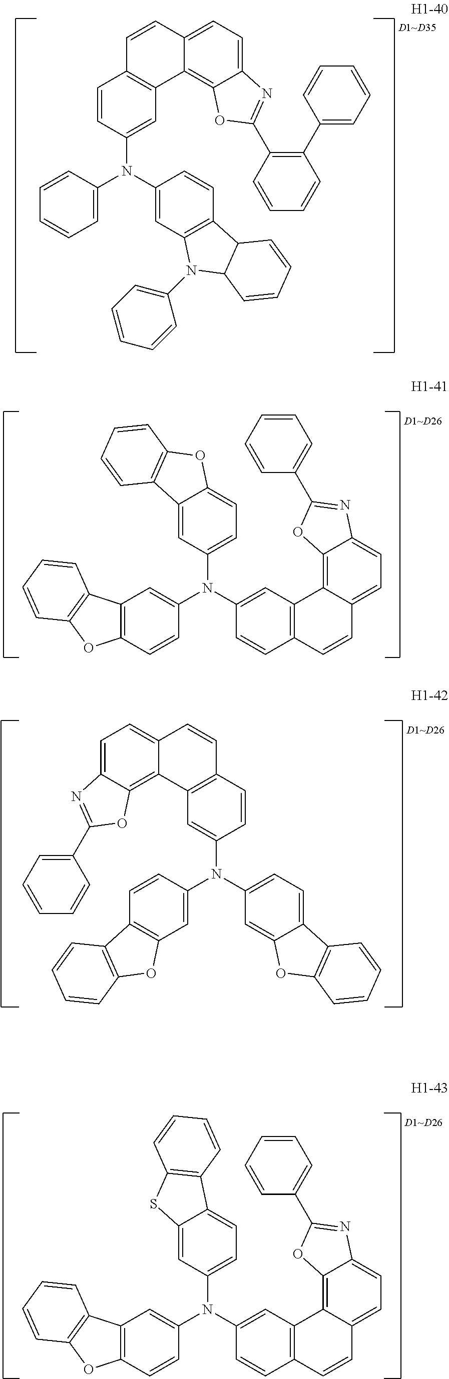 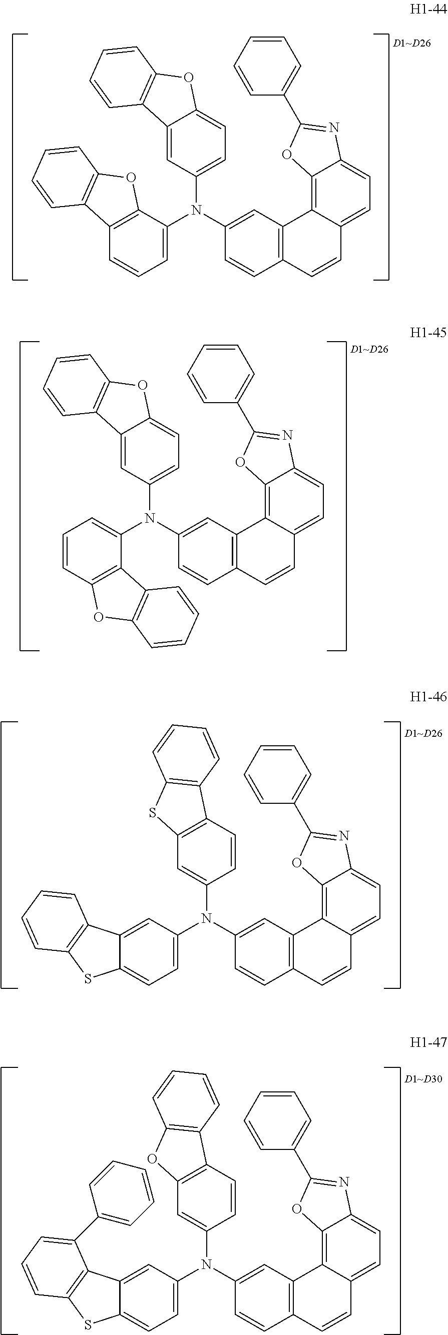 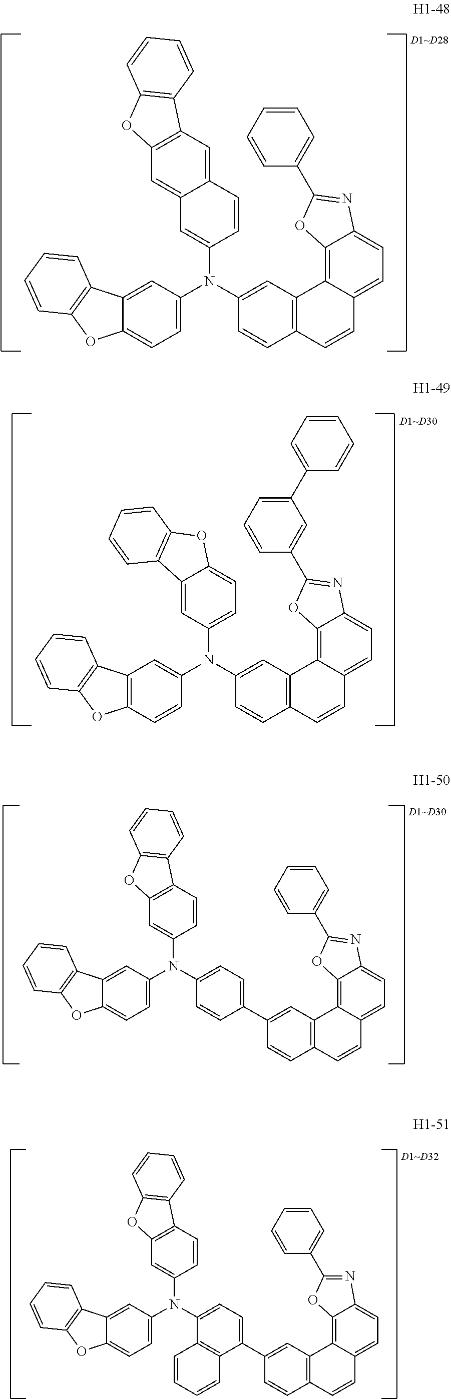 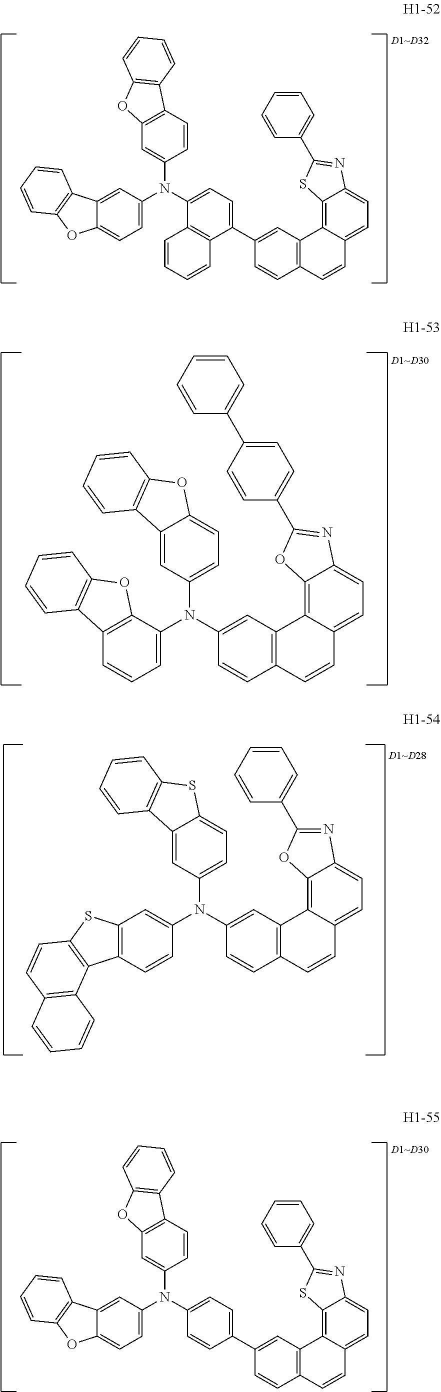 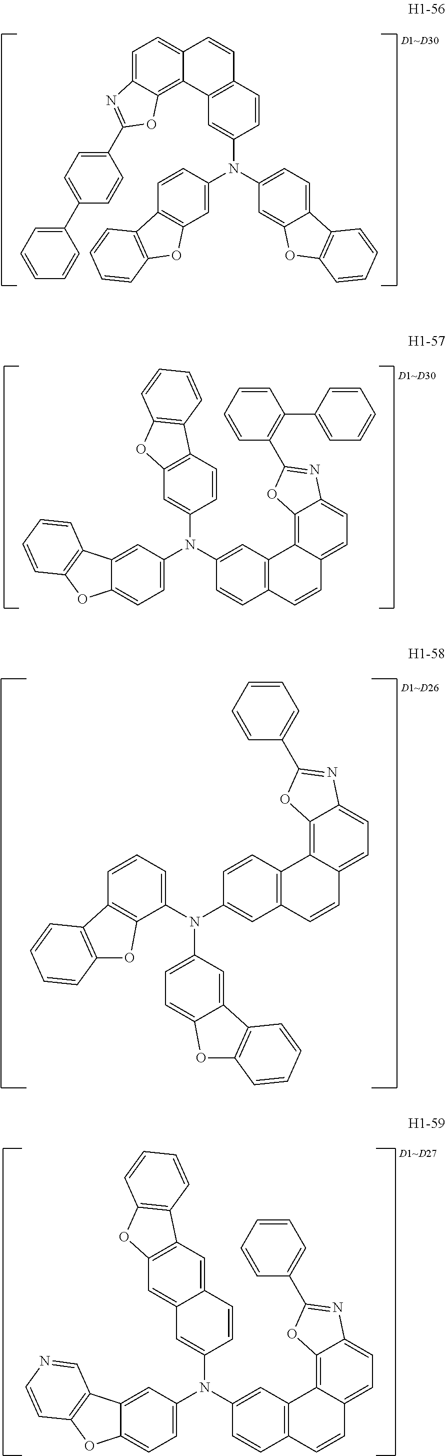 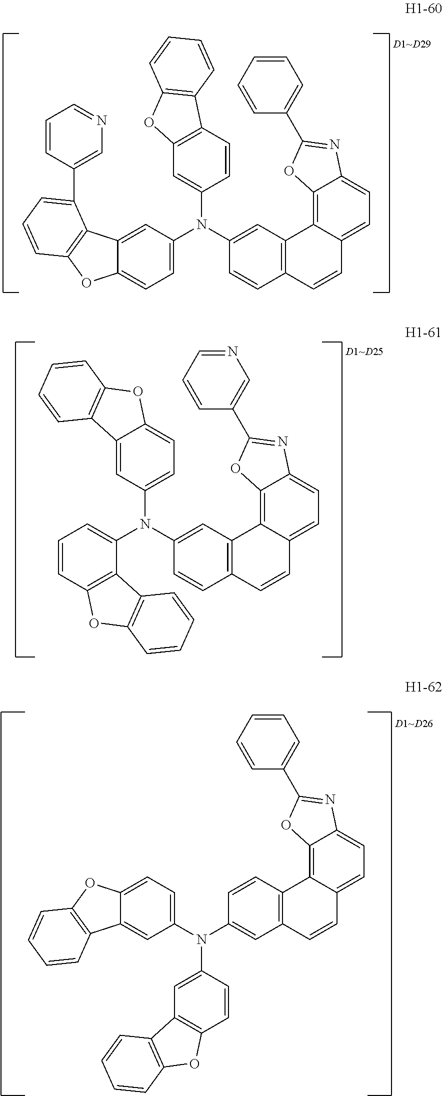 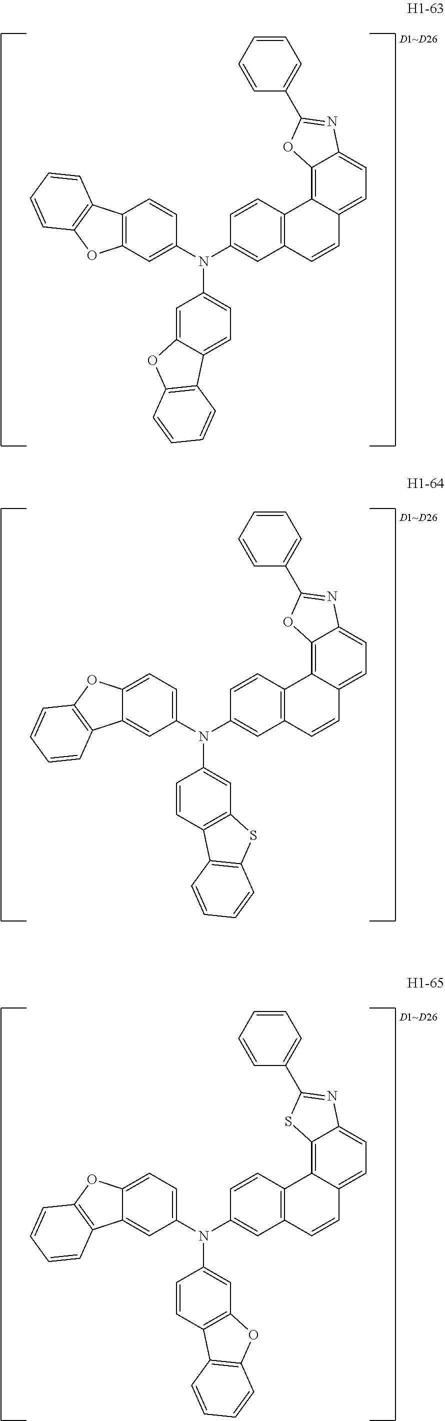 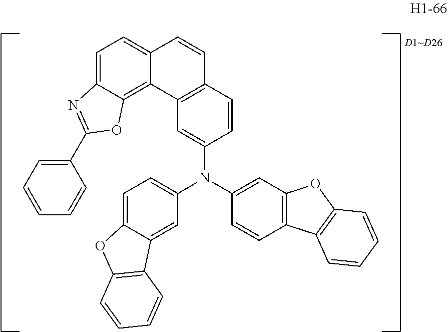 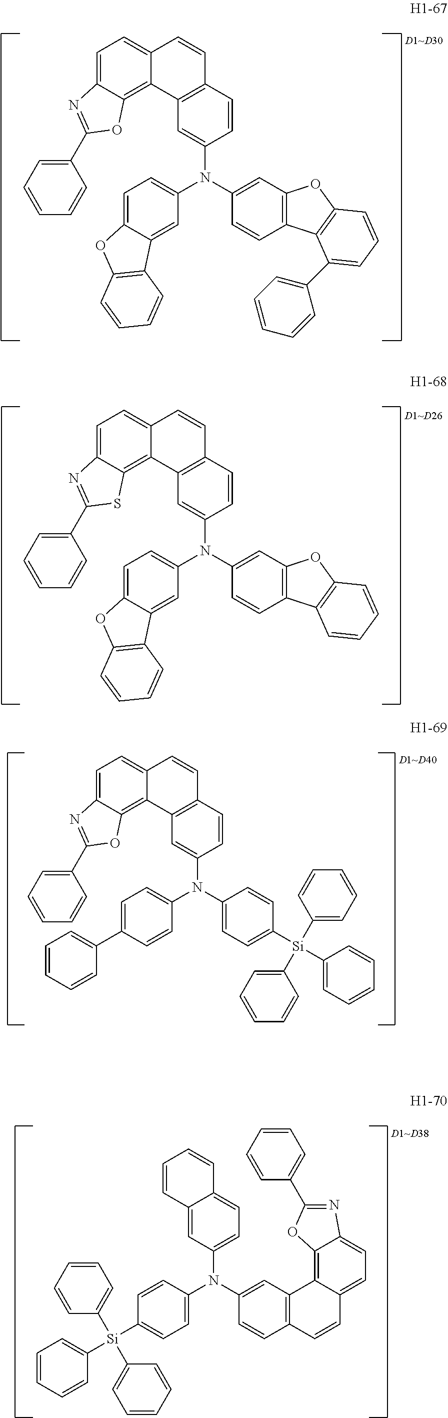 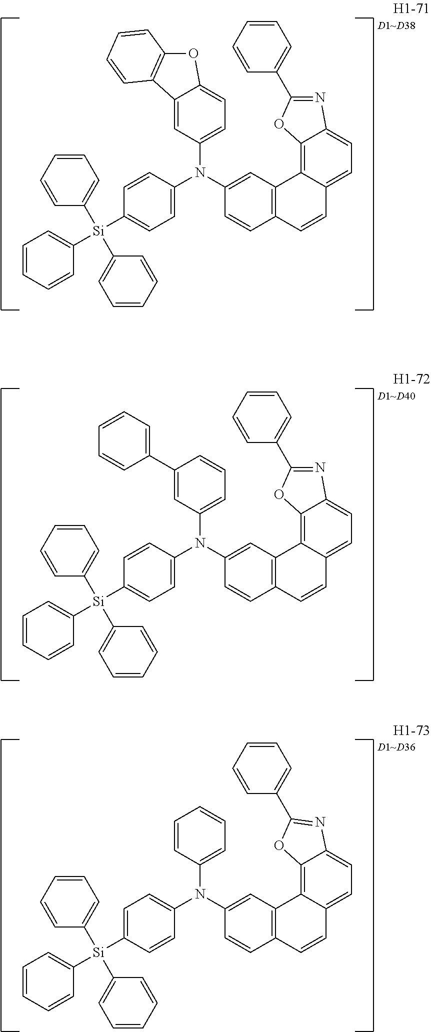 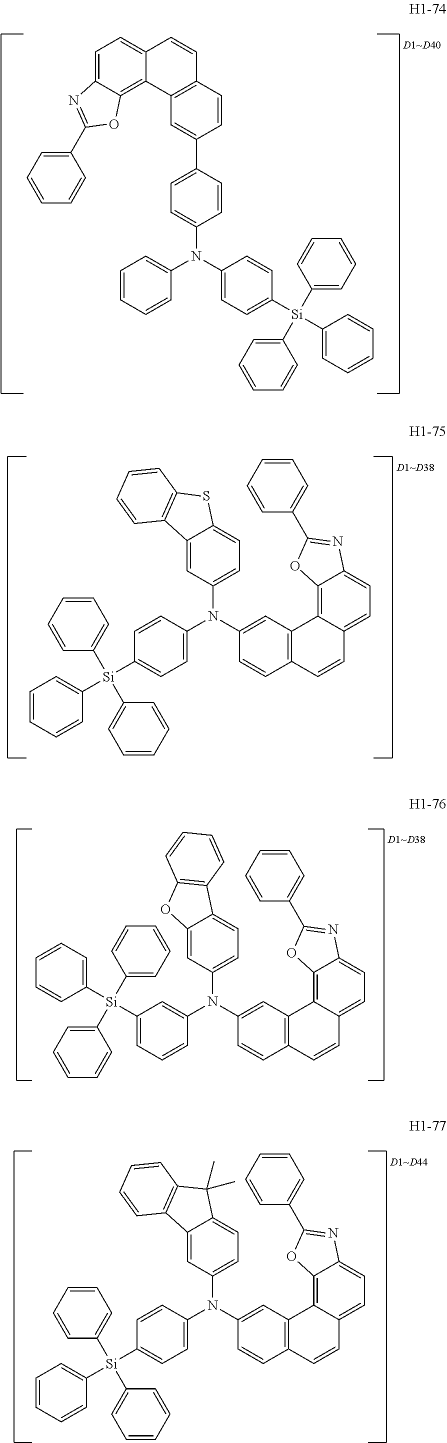 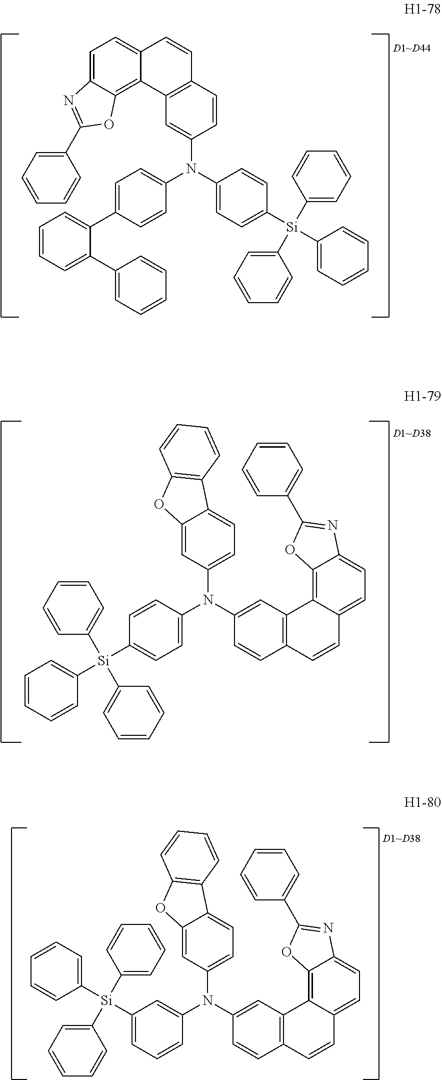 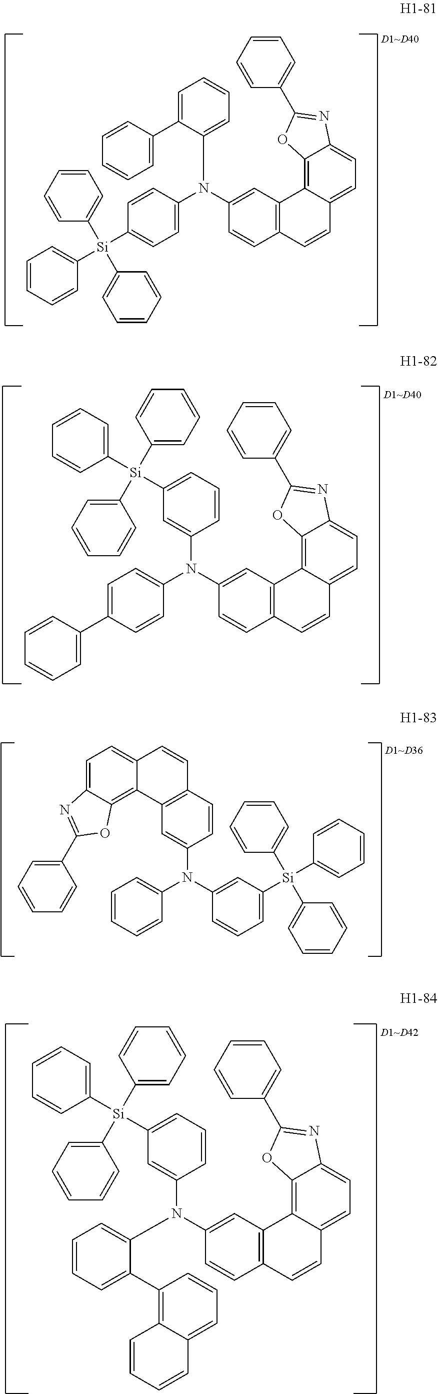 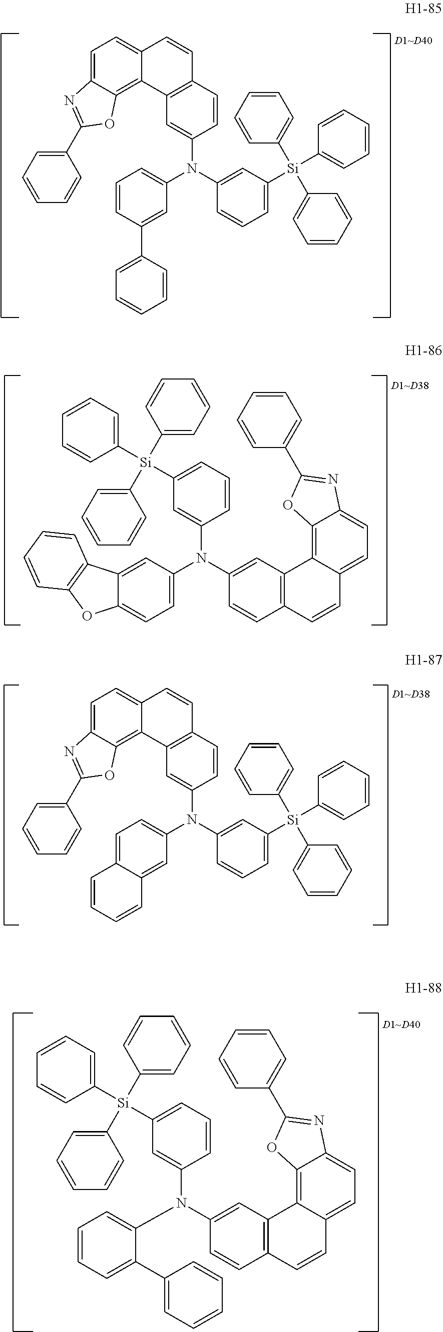 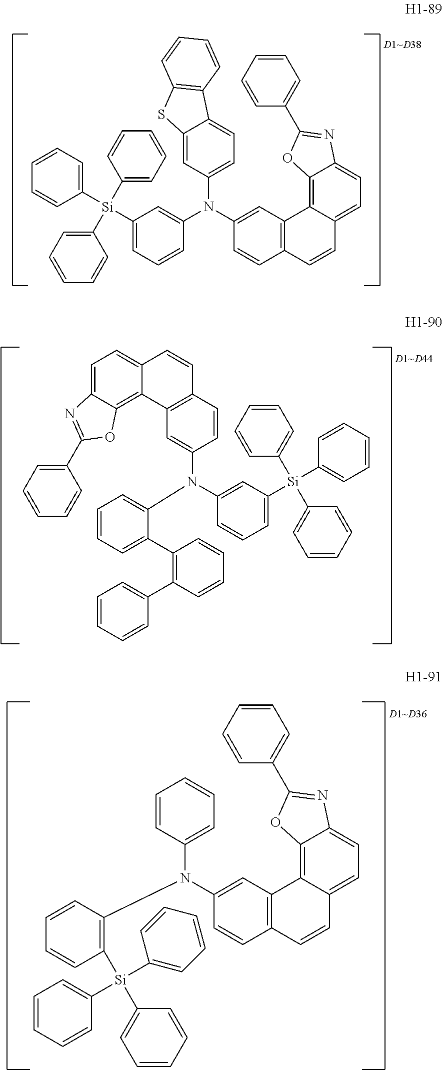 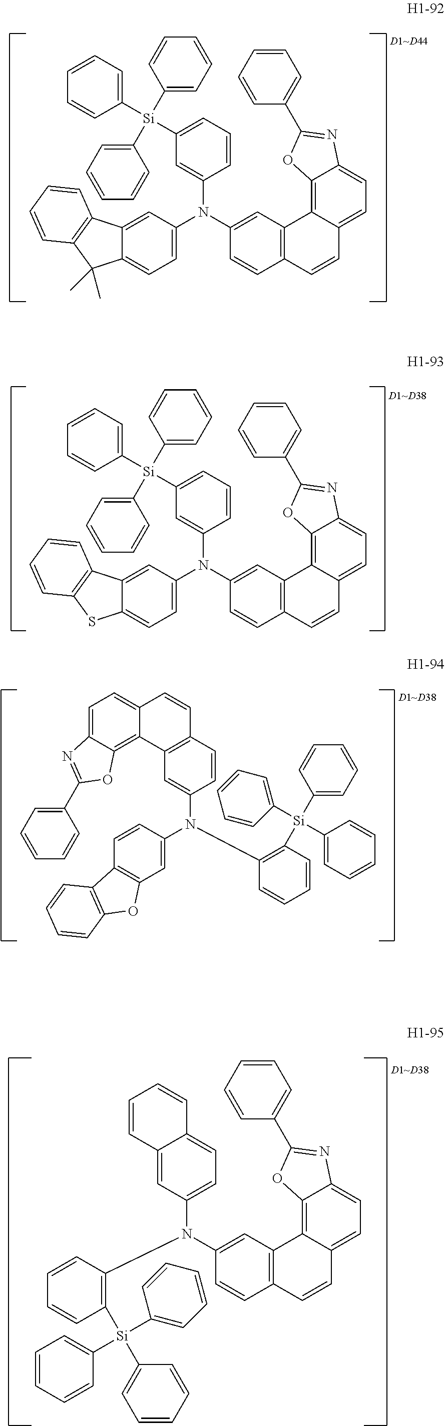 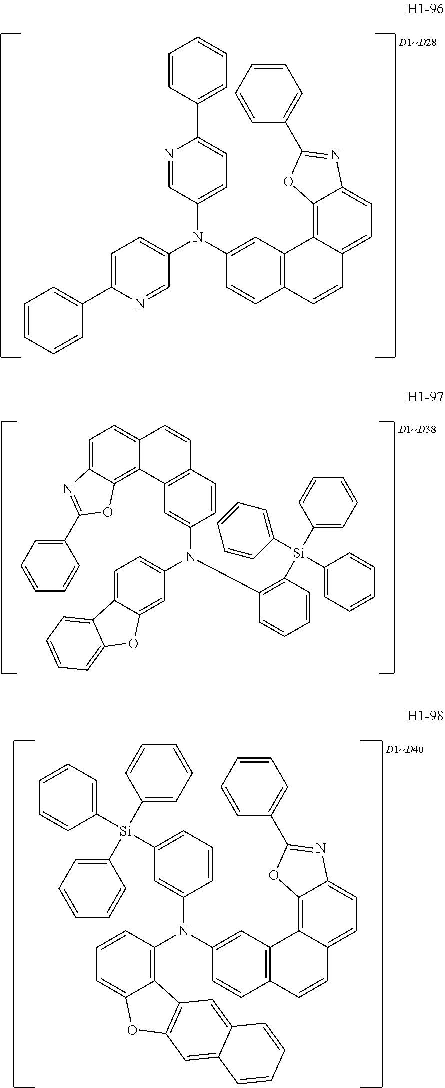 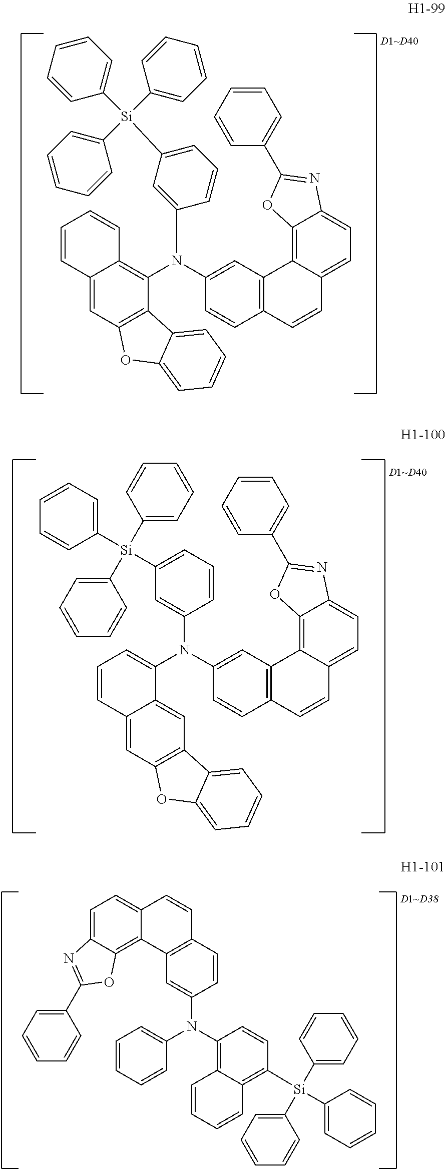 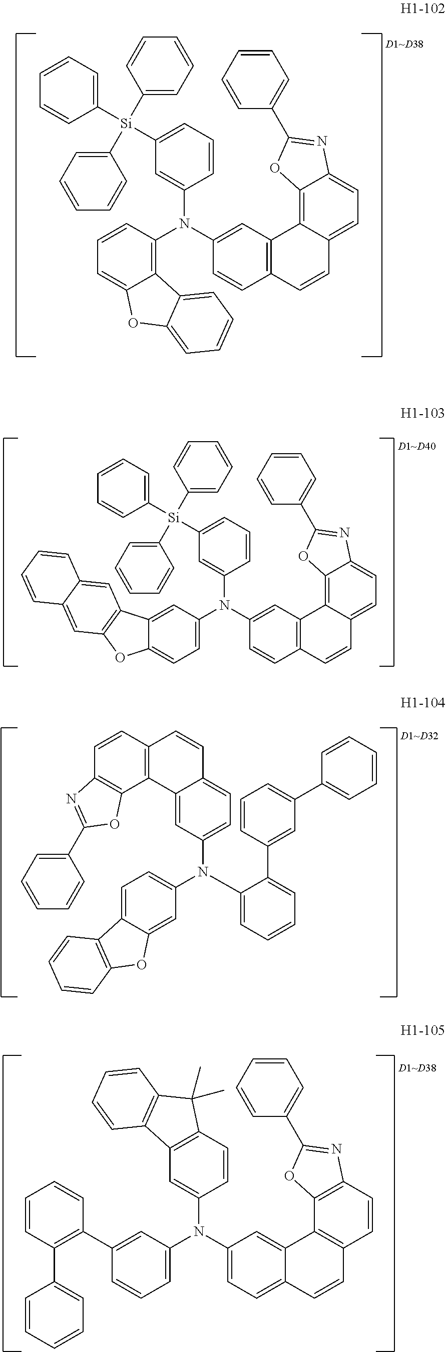 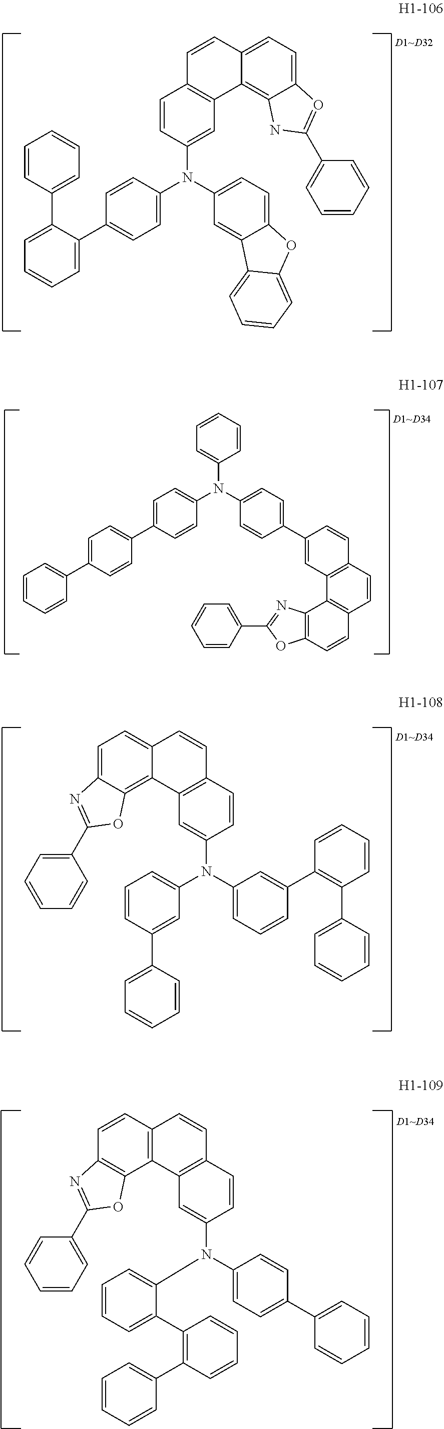 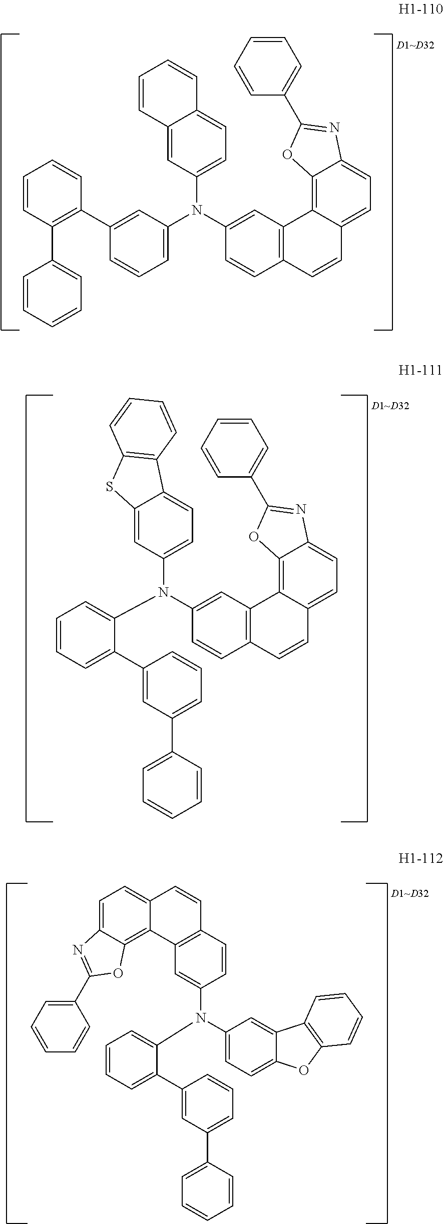 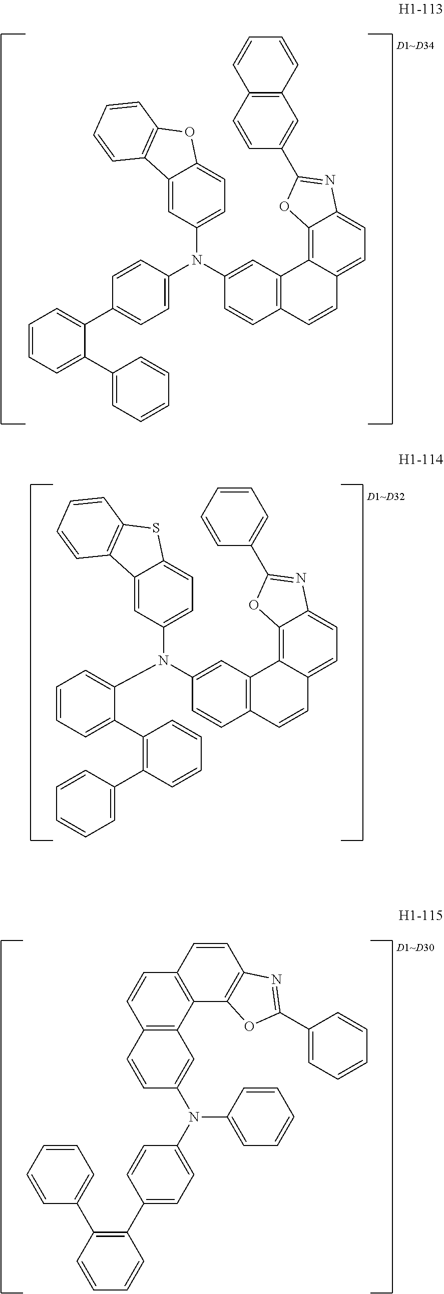 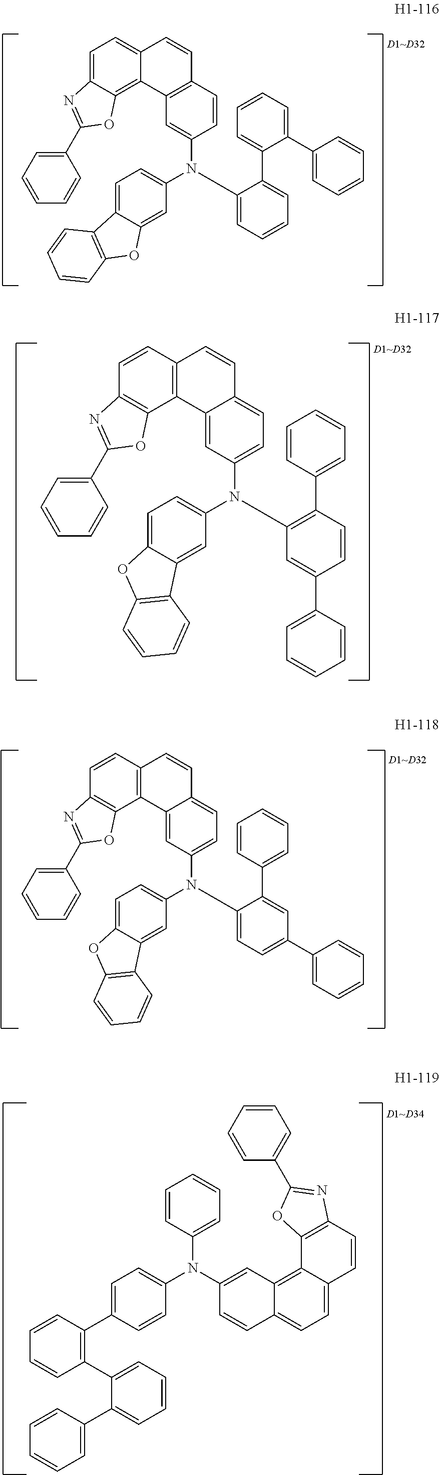 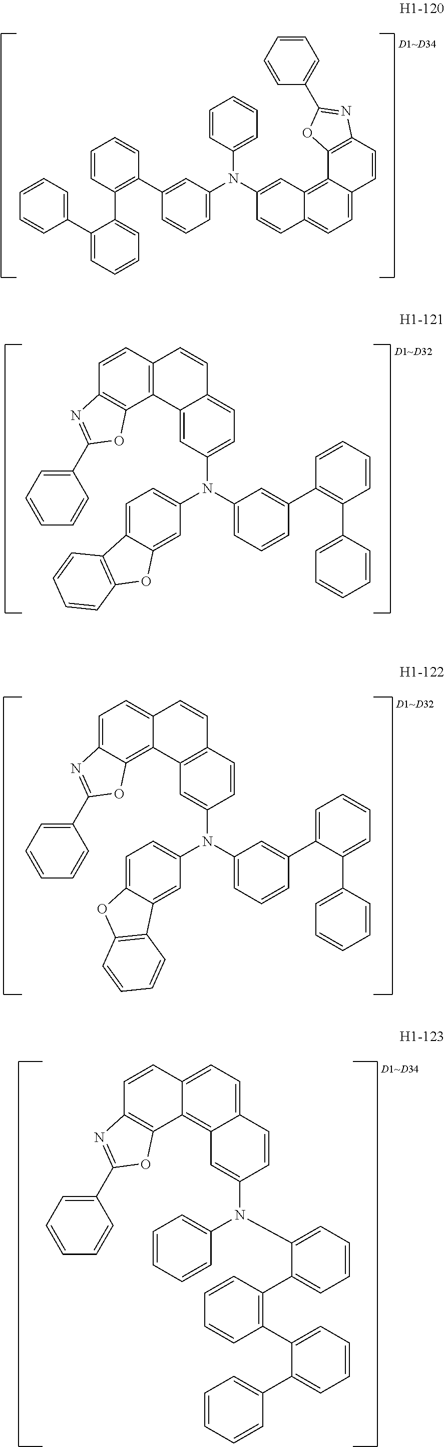 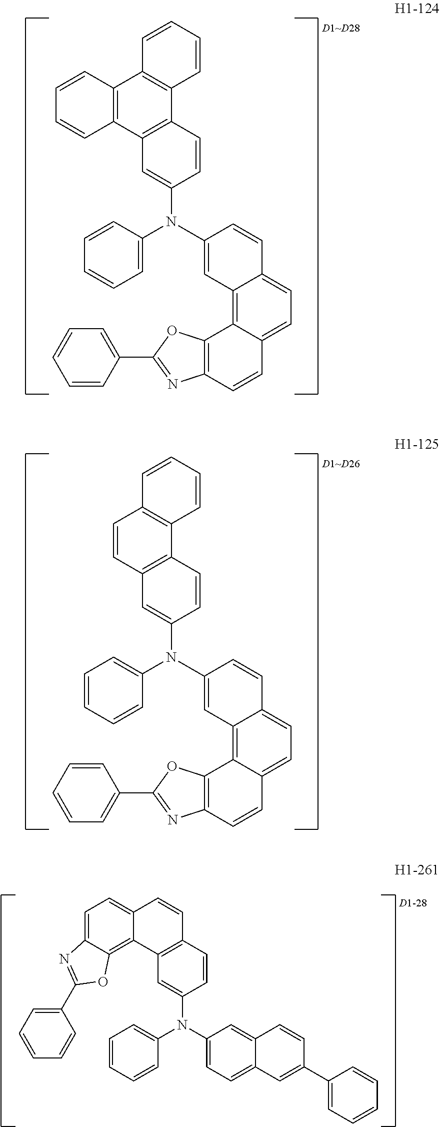 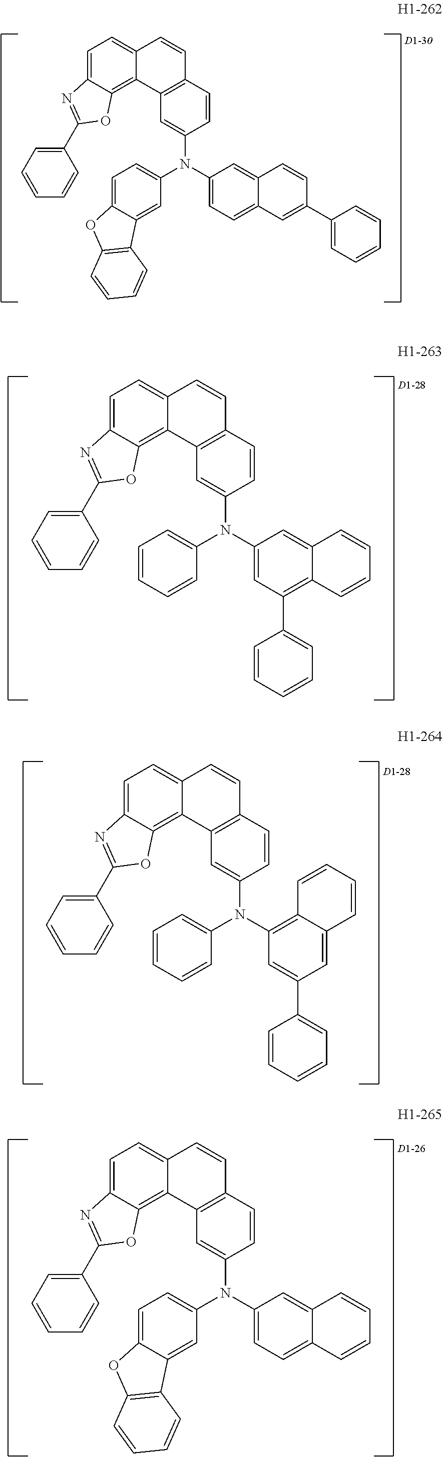 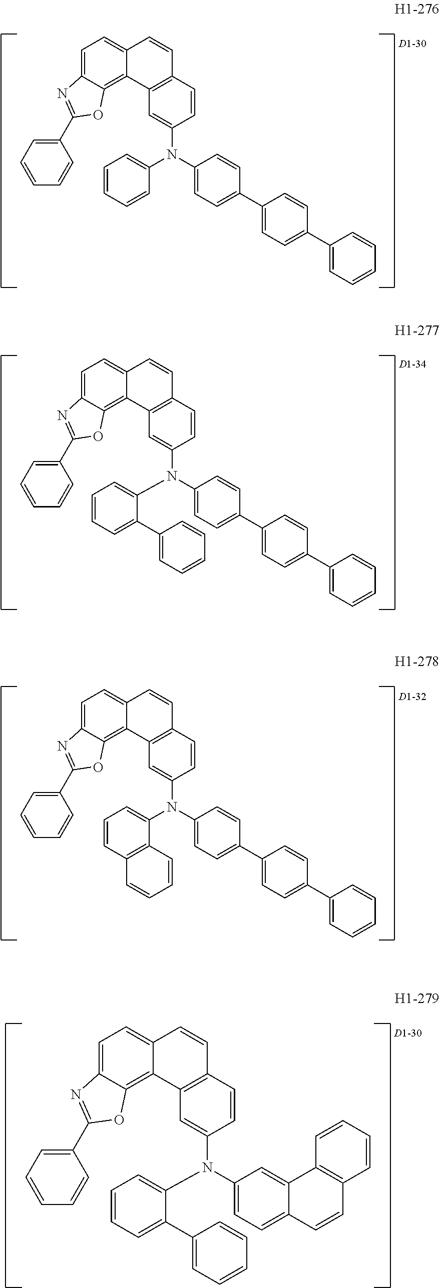 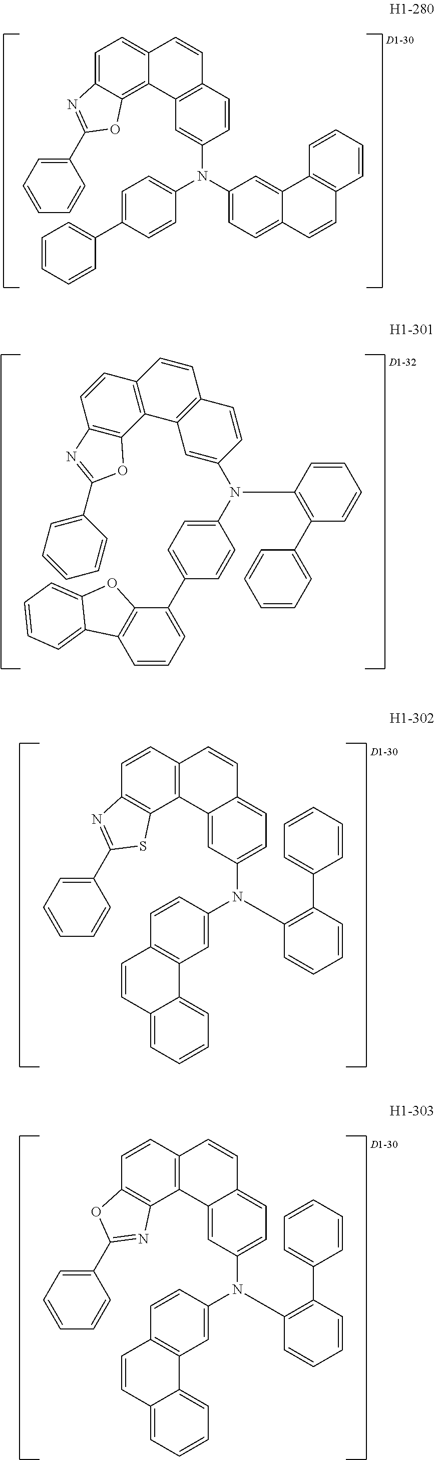 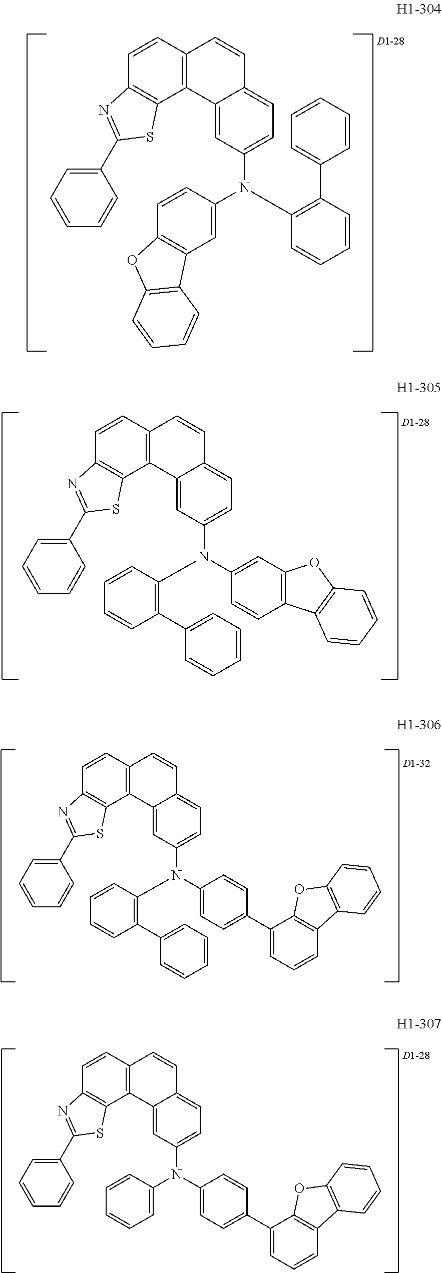 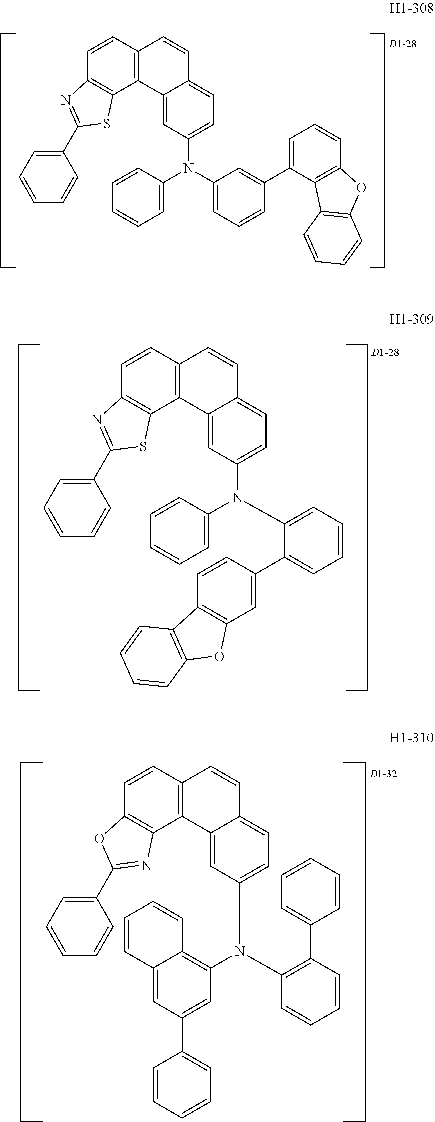 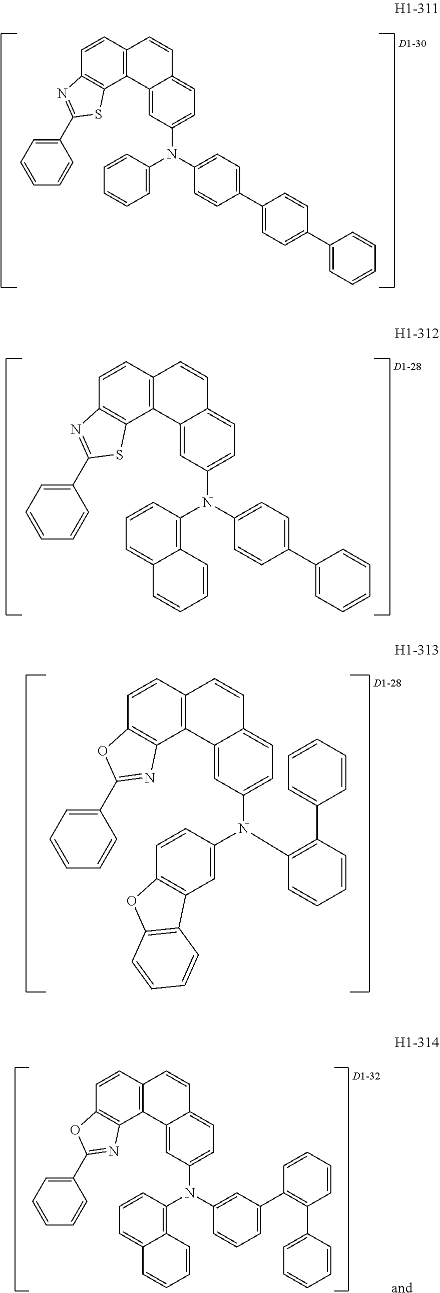 [chemical structure]
